(19) United States
(12) Patent Application Publication
(10) Pub. No.: US 2024/0060136 A1
(43) Pub. Date: Feb. 22, 2024

(54) METHODS FOR DETECTING AND PREDICTING GRADE 3 CERVICAL EPITHELIAL NEOPLASIA (CIN3) AND/OR CANCER

(71) Applicant: UCL Business Ltd, London (GB)

(72) Inventors: Martin Widschwendter, London (GB); James Barrett, London (GB); Allison Jones, London (GB); Iona Evans, London (GB)

(21) Appl. No.: 18/009,957

(22) PCT Filed: Jun. 17, 2021
(86) PCT No.: PCT/GB2021/051537

§ 371 Date: Dec. 12, 2022

(30) Foreign Application Priority Data
Jun. 17, 2020 (GB) 2009225.0
May 25, 2021 (GB) 2107421.6

(51) Int. Cl. C12Q 1/6886; C12Q 1/70; C12Q 1/6837

(52) U.S. Cl. CPC C12Q 1/6886; C12Q 1/708; C12Q 1/6837; C12Q 2600/154; C12Q 1/6806

(57) ABSTRACT

The present invention relates to assays for predicting the presence, absence or development of CIN3 and/or cancer, particularly cervical or endometrial cancer, most preferably cervical cancer, in an individual, particularly cervical and endometrial cancer, by determining the methylation status of certain CpGs in a population of DNA molecules in a sample which has been taken from the individual, deriving an index value based on the methylation status of the certain CpGs, and predicting the presence, absence or development of CIN3 and/or cancer, particularly cervical or endometrial cancer, most preferably cervical cancer, in the individual based on the cancer index value. The invention further relates to a method of treating and/or prevention of cancer in an individual, particularly cervical and endometrial cancer, the method comprising assessing the presence, absence or development of CIN3 and/In or cancer, particularly cervical or endometrial cancer, most preferably cervical cancer, in an individual by performing the assays of the invention, followed by administering one or more therapeutic treatments or measures to the individual based on the assessment. The invention further provides a method of monitoring the CIN3 and/or cancer status of an individual according to changes in the individual's cancer index value over the course of time. The invention further relates to arrays which are suitable for performing the assays of the invention.

### SEQUENCE LISTING

The instant application contains a Sequence Listing which has been submitted electronically in ASCII format and is hereby incorporated by reference in its entirety. Said ASCII copy, created on Jun. 9, 2021, is named ‘Sequence listing as filed 17 Jun. 2021 N417235WO MGW JAS.txt’ and is 3,022,158 bytes in size.

### FIELD OF THE INVENTION

The present invention relates to assays for predicting the presence, absence or development of grade 3 cervical intra-epithelial neoplasia (CIN3) and/or cancer in an individual, particularly cervical or endometrial cancer, most preferably cervical cancer, by determining the methylation status of certain CpGs in a population of DNA molecules in a sample which has been taken from the individual, deriving an index value based on the methylation status of the certain CpGs, and predicting the presence, absence or development of CIN3 and/or cancer, particularly cervical or endometrial cancer, most preferably cervical cancer, in the individual based on the cancer index value. The invention further relates to a method of treating and/or preventing CIN3 and/or cervical cancer in an individual, the method comprising assessing the presence, absence or development of CIN3 and/or cancer, particularly cervical or endometrial cancer, most preferably cervical cancer, in an individual by performing the assays of the invention, followed by administering one or more therapeutic or preventative treatments or measures to the individual based on the assessment. The invention further provides a method of monitoring the CIN3 and/or cancer status of an individual according to changes in the individual's cancer index value over the course of time. The invention further relates to arrays which are suitable for performing the assays of the invention.

The project leading to this application has been funded by the European Commission's Horizon 2020 Research and Innovation Action, H2020 FORECEE under Grant Agreement No. 634570, the European Commission's Horizon 2020 European Research Council Executive Agency, H2020 BRCA-ERC under Grant Agreement No. 742432 as well as the charity, The Eve Appeal.

### BACKGROUND TO THE INVENTION

Cervical cancer screening has been the most successful personalised cancer prevention strategy to date; the screening aims to identify women with a pre-invasive lesion, which is then surgically excised.

At this point in time, the majority of countries are changing screening from cytology to HPV testing as the primary screen and utilising cytology to triage HPV+ve women for colposcopic assessment. However, several challenges remain for HPV-based screening:
(1) Low specificity of HPV: HPV is highly prevalent in cytology-ye women which is up to 24% depending on age and country and even in HPV-vaccinated women the prevalence of HPV infection is approximately 5%.
(2) Low sensitivity of cytology in HPV+ve women: approximately 31% of CIN3+(CIN3 or invasive cancers) in HPV+ve women are primarily cytology-ye and are only diagnosed with CIN3+12 months later due colposcopy triggered by HPV-persistence or becoming cytology+ve.
(3) The participation rates amongst European countries vary between 40.5% and 81.4% and efforts to increase participation are essential. A recent meta-analysis provided strong evidence that self-sampling has a consistently higher acceptance over clinician sampling. HPV testing shows comparable results in self-versus clinician-collected samples, but the fact that less than 60% of women who provide a self-collected sample show compliance with follow up recommendations indicates that a test other than cytology to triage women based on the same self-collected sample which tested HPV+ve is required in order to increase acceptability and eventually participation rates.

To date, the best performing triage algorithm for HPV+ve women with respect to diagnosing CIN3+ is dual immunostaining for p16/Ki-67; both cytology and dual staining have a specificity of 75% but dual staining is more sensitive (74.9%) when compared with conventional cytology (51.9%).

The present inventors, along with other investigators, have previously shown the feasibility of utilising DNA methylation markers in order to identify women with pre-invasive or invasive cancers. The clinical use of DNA methylation markers to identify women at high risk for CIN3+ has been hindered by several factors:
(i) An unacceptably low sensitivity in detecting CIN3 or CIN 3+, particularly in young women who have a substantially higher prevalence of HPV. These women therefore have an increased need for triage testing with high performance, for instance, the GynTect test (which utilises DNA methylation of six genes) has a sensitivity for CIN3 which varies between 35% in <30 year old and 76% in ≥30 year old women and the sensitivity in detecting CIN3+ for the QIAsure (which uses methylation of two genes) varies between 37.5% in <30 year old and 89.3% in ≥30 year old women who were referred with an abnormal cytology.
(ii) A low specificity which is also age dependent and comparable to cytology ranging from 76.6% in ≥30 year old women to 87.8% in <30 year old women.
(iii) A lack of data prevents judgement as to whether a DNA methylation marker or marker panel is capable of identifying HPV+ve women which, despite being cytology-ye at the time of assessment, go on to develop CIN3+ in succeeding years.

Using a cohort-based nested case/control setting, the inventors have developed and validated a DNA methylation signature (called Women's cancer risk IDentification CIN3 index, WID-CIN3-index) in cervical smear samples which is capable of both diagnosing and predicting the future risk of CIN3+ in an individual as well as cancer, particularly cervical or endometrial cancer, most preferably cervical cancer.

### SUMMARY OF THE INVENTION

The current inventors set out to understand whether DNAme (DNA methylation) profiles may be used to detect the presence or absence of grade 3 cervical intra-epithelial neoplasia (CIN3) and/or cervical cancer. The inventors also set out to understand whether said DNAme profiles may be associated with the development of CIN3 and/or cancer, particularly cervical or endometrial cancer, most preferably cervical cancer, and therefore whether such profiles may be capable of functioning as surrogate markers for individual stratification purposes in connection with CIN3 and/or cervical cancer.

In this regard the inventors have succeeded in developing assays involving “cancer index values” which are derived from and associated with DNAme profiles established from samples comprising epithelial cells. The sample may particularly be derived from the cervix, the vagina, the buccal area, blood and/or urine. The sample is preferably a cervical liquid-based cytology sample, and more preferably a cervical smear sample and which values can be used to stratify the individual in connection with cancer. A preferred sample for use in any of the assays described and defined herein is a cervical tissue sample. A particularly preferred sample for use in any of the assays described and defined herein is a cervical smear sample.

The cancer index value is determined from data relating to the methylation status of one or more CpGs in a panel of CpGs as further defined and described herein. CpGs of the panel are methylation sites in DNA from cells derived from/obtained from samples from tissue in which the native tissue structure is preserved e.g. a biopsy, or a sample comprising exfoliated cells from a tissue surface. The samples may comprise epithelial cells. The sample may particularly be derived from the cervix, the vagina, the buccal area, blood and/or urine. The sample is preferably a cervical smear sample and more preferably a cervical liquid-based cytology sample which can be collected by a health care professional or by a women herself (self-collection).

For the purposes of the present invention, the cancer index value may be used interchangeably herein with “WID-CIN-Index”, “WID-Index”, “cancer index”, “index” or “index value” (WID=women's risk identification). Furthermore, any reference to a cancer index value in the context of the present invention, may be equally used for the assessment of the presence, absence or development of CIN3 and/or cancer, particularly cervical or endometrial cancer, most preferably cervical cancer, in an individual.

Based on studies with patients known to be CIN3-negative and/or free of cervical cancer, the inventors have established cancer index values, using specific panels of CpGs, which have been determined to be associated with/characteristic of cervical tissue which is CIN3-negative and/or negative for cervical cancer. Based on studies with patients known to possess CIN3 and/or cervical cancer, the inventors have established cancer index values which have been determined to be associated with/characteristic of cervical tissue which is positive for CIN3 and/or cervical cancer. Based on studies with patients known to be CIN3-negative and/or free of cervical cancer, wherein the same patients when assayed between one to four years later are subsequently shown determined to be CIN3-positive and/or positive for cervical cancer, the inventors have established cancer index values which have been determined to be associated with/characteristic of cervical tissue which is positive for CIN3 and/or cervical cancer.

Thus, the inventors have been able to establish cancer index values, using specific panels of CpGs, which can characterize an individual as having CIN3 and/or cancer or not having CIN3 and/or cancer, or having a high risk of CIN3 and/or cancer development. The cancer is preferably cervical or endometrial cancer, most preferably cervical cancer.

By determining the methylation profile-based cancer index value from a sample derived from the individual, the individual may be seen to possess a cancer index value which correlates with those possessed by individuals which are known, via the inventor's studies described herein, to be CIN3 positive or negative and/or cervical cancer positive or negative, or to become CIN3 positive or negative and/or cervical cancer positive or negative. Such correlations have been determined with a high degree of statistical accuracy, particularly with respect to parameters relevant to biological assays such as receiver operating characteristics (ROC) sensitivity and specificity, as well as area under the curve (AUC). Accordingly, by determining the cancer index value from a sample from a given individual, the individual may be determined to possess cervical tissue that is positive for CIN3 and/or cancer, i.e. the individual is diagnosed as having CIN3 and/or cervical cancer. Conversely, by determining the cancer index value from a sample from a given individual, the individual may be determined to possess cervical tissue which is negative for CIN3 and/or cancer, i.e. the individual is diagnosed as not having CIN3 and/or cervical cancer.

Assessment of CIN3 in accordance with assays of the invention may identify individuals likely to develop CIN3 in the future, particularly within about four years from the date of to the first assessment of the individual with the one or more of the assays described herein.

Assessment of the development of cancer in accordance with the assays of the invention may refer to assessing progression or worsening of a pre-existing cancer lesion in an individual. Assessment of the development of cancer in accordance with the assays of the invention may refer to predicting the likelihood of recurrence of cancer.

The observations described herein establish that the cancer index value, as further described and defined herein, is dynamic and can change over the course of time. The cancer index value may therefore be used to monitor an individual's cancer status and risk of cancer development. Moreover, the cancer index value may be used to monitor the efficacy of cancer treatments being administered to an individual, including therapeutic treatments and preventative treatments.

Accordingly, in the context of the present invention, by determining the cancer index value from a sample from a given individual it is possible to assess the presence, absence or development of CIN3 and/or cancer, particularly cervical or endometrial cancer, most preferably cervical cancer, in an individual, or in other words to stratify the individual for cancer. In the context of the present invention, stratification for cancer is the process of categorizing the individual as being a member of a group of individuals who possess a phenotype in connection with cancer, including the presence or absence of cancer in the individual, or the development of cancer, i.e. by having epithelial cells, particularly derived from the cervix, the vagina, the buccal area, blood and/or urine, more preferably a cervical smear sample and even more preferably a cervical liquid-based cytology sample.

As explained herein, the assay methods of the invention are based on a cancer index value derived from a methylation profile from DNA originating from cells, particularly derived from the cervix, the vagina, the buccal area, blood and/or urine, more preferably a cervical liquid-based cytology sample, and even more preferably a cervical smear sample. Accordingly, the assays provide means for correlating an epithelial cell, or most preferably a cervical smear sample-derived DNA methylation profile with a status connected with cervical or endometrial cancer ranging from the individual being cancer negative, to the individual being cancer positive, with high statistical accuracy. Because the assays of the invention provide a correlation between the methylation profile and the disease status, the skilled person will appreciate that as part of the stratification process and outcome, disease status is assigned on the basis of a likelihood. As such, the methods of the invention provide assays which are predictive of an individual's status with respect to cancer. The assays of the invention accordingly provide means for predicting the presence or absence of cancer in an individual. The assays of the invention accordingly also provide means for predicting the development of cancer in an individual. The assays of the invention can provide means for predicting the development of cancer in an individual since the inventors have demonstrated that specific cancer index values can define cervical and endometrial tissue which is cancer negative, whilst others can define cervical and endometrial tissue which is cancer positive, and since the specific cancer index values may be dynamic and thereby increased in association with tumour stage and further increased cancer risk factors such as the women being post-menopausal, the values may be subject to change along a scale of cancer risk.

Whilst disease status may be assigned on the basis of a likelihood, the inventors have demonstrated herein that correlations between DNA methylation profile and cancer status using cancer index values can be achieved with a very high degree of statistical accuracy using parameters relevant to biological assays, as described further herein. As such, the assays of the invention provide means for predicting the presence or absence of cancer in an individual and for predicting the development of cancer in an individual, and for stratifying an individual for cancer, and wherein the prediction/stratification can be defined to be statistically highly reliable and robust. This in turn means that the prediction/stratification can be made with a high level of confidence. The assays of the invention can be defined to be statistically accurate by means known in the art, as further described and defined herein. The assays of the invention can be defined according to parameters relating to their statistical specificity and sensitivity. These parameters define the likelihood of false positive and false negative test results. The lower the proportion of false positive and false negative test results the more statistically accurate the assay becomes. In this regard the inventors have established CpG panels, as described and defined further herein, wherein the methylation status of CpGs in the panel can be used to establish cancer index values such that the assays produce statistically accurate predictions of cancer status. Accordingly, the inventors have determined that the assays described herein may be defined according to statistical parameters such as percentage specificity and sensitivity and also by receiver operating characteristics (ROC) area under the curve (AUC). All such means are known in the art and are known to be defined measures of statistical accuracy for biological assays such as those described and defined herein.

Thus the methods of the invention provide assays which can be used, with a high degree of statistical accuracy, to predict the presence, absence or development of CIN3 and/or cancer, particularly cervical and endometrial cancer, most preferably cervical cancer. The methods of the invention provide assays which can be used, with a high degree of statistical accuracy, to stratify an individual with respect to cancer status. Accordingly, the methods of the invention provide useful information to individuals and their physicians concerning patient cancer status. This information may help inform actual therapeutic treatment measures if the presence of cancer is identified in the individual. The information may help to monitor the progress of therapeutic treatment measures in the individual by monitoring changes in the cancer index value over the course of a period of time. The information may help to monitor the progress of prophylactic or preventative treatment measures in the individual by monitoring changes in the cancer index value over the course of a period of time. As such the methods of the invention offer significant advantages in the personalized prevention and early detection as well as treatment and management of cancer in individuals.

Accordingly, the invention provides an assay for assessing the presence, absence or development of grade 3 cervical intra-epithelial neoplasia (CIN3) and/or cancer in an individual, the assay comprising:
1. providing a sample which has been taken from the individual, the sample comprising a population of DNA molecules;
2. determining in the population of DNA molecules in the sample the methylation status of a panel of:
   i. one or more CpGs selected from a panel of CpGs identified in SEQ ID NOs 1 to 5000 wherein the CpGs are identified at nucleotide positions 61 to 62; and/or
   ii. one or more CpGs selected from within a panel of one or more Differentially Methylated Regions (DMRs) defined by SEQ ID NOs 5001 to 5418, wherein the CpGs are denoted by CG;
3. deriving a cancer index value based on the methylation status of the one or more CpGs in the panel; and
4. assessing the presence, absence or development of CIN3 and/or cancer, preferably wherein the cancer is cervical cancer, in the individual based on the cancer index value;
wherein the assay is characterised as having an area under the curve (AUC) of 0.60 or more as determined by receiver operating characteristics (ROC).

The assay of the invention may be performed as above and additionally wherein the panel of one or more CpGs comprises at least 500 CpGs selected from the CpGs identified at nucleotide positions 61 to 62 in SEQ ID NOs 1 to 5000, preferably wherein the assay is characterised as having an AUC of at least 0.80.

The assay of the invention may be performed as above and additionally wherein the panel of one or more CpGs comprises at least the CpGs identified in SEQ ID NOs 1 to 500 and identified at nucleotide positions 61 to 62, preferably wherein the assay is characterised as having an AUC of at least 0.92.

The assay of the invention may be performed as above and additionally wherein the panel of one or more CpGs comprises at least 1000 CpGs selected from the CpGs identified at nucleotide positions 61 to 62 in SEQ ID NOs 1 to 5000, preferably wherein the assay is characterised as having an AUC of at least 0.80.

The assay of the invention may be performed as above and additionally wherein the panel of one or more CpGs comprises at least the CpGs identified in SEQ ID NOs 1 to 1000 and identified at nucleotide positions 61 to 62, preferably wherein the assay is characterised as having an AUC of at least 0.92.

The assay of the invention may be performed as above and additionally wherein the panel of one or more CpGs comprises at least 1500 CpGs selected from the CpGs identified at nucleotide positions 61 to 62 in SEQ ID NOs 1 to 5000, preferably wherein the assay is characterised as having an AUC of at least 0.82.

The assay of the invention may be performed as above and additionally wherein the panel of one or more CpGs comprises at least the CpGs identified in SEQ ID NOs 1 to 1500 and identified at nucleotide positions 61 to 62, preferably wherein the assay is characterised as having an AUC of at least 0.92.

The assay of the invention may be performed as above and additionally wherein the panel of one or more CpGs comprises at least 2000 CpGs selected from the CpGs identified at nucleotide positions 61 to 62 in SEQ ID NOs 1 to 5000, preferably wherein the assay is characterised as having an AUC of at least 0.81.

The assay of the invention may be performed as above and additionally wherein the panel of one or more CpGs comprises at least the CpGs identified in SEQ ID NOs 1 to 2000 and identified at nucleotide positions 61 to 62, preferably wherein the assay is characterised as having an AUC of at least 0.92.

The assay of the invention may be performed as above and additionally wherein the panel of one or more CpGs comprises at least the 5000 CpGs identified at nucleotide positions 61 to 62 in SEQ ID NOs 1 to 5000, and further wherein the assay is characterised as having an AUC of at least 0.92.

The assay of the invention may be performed as above and additionally wherein the step of determining in the population of DNA molecules in the sample the methylation status of the one or more CpGs in the panel comprises determining a β value of each CpG.

The assay of the invention may be performed as above and additionally wherein the step of deriving the cancer index value based on the methylation status of the one or more CpGs in the panel comprises:
1. providing a methylation β-value data set comprising the methylation β-values for each CpG in the panel;
2. providing a mathematical model capable of generating the cancer index from the methylation β-value data set; and
3. applying the mathematical model to the methylation β-value data set, thereby generating the cancer index.

The assay of the invention may be performed as above and additionally wherein the cancer index value is a WID-CIN-Index cancer index value, and wherein the mathematical model which is applied to the methylation β-value data set to generate the cancer index is an algorithm according to the following formula:

$$WID - CIN - index = {\sum }_{i = 1}^{n} \frac{μ - {w}_{i} {β}_{i}}{σ}$$

wherein:
1. β₁, . . . , βₙ are methylation beta-values (between 0 and 1);
2. w₁, . . . , w₅₀₀₀ are real valued coefficients;
3. μ and σ are real valued parameters used to scale the index; and
4. n refers to the number of CpGs in the panel of one or more CpGs; preferably wherein the cancer is cervical cancer.

The assay of the invention may be performed as above and additionally wherein when the cancer index value for the individual is about −0.331 or more, the individual is assessed as having CIN3 and/or cancer or as having a high risk of CIN3 and/or cancer development, or wherein when the cancer index value for the individual is less than about −0.331, the individual is assessed as not having CIN3 and/or cancer or as having a low risk of CIN3 and/or cancer development, preferably wherein:
a. the panel of one or more CpGs comprises at least 500 of the CpGs identified at nucleotide positions 61 to 62 in SEQ ID NOs 1 to 5000, and wherein the sensitivity is at least 87% and the specificity is at least 49%;
b. the panel of one or more CpGs comprises at least the CpGs defined by SEQ ID NOs 1 to 500 and identified at nucleotide positions 61 to 62, and wherein the sensitivity is at least 86% and specificity is at least 77%;
c. the panel of one or more CpGs comprises at least the CpGs defined by SEQ ID NOs 1 to 1000 and identified at nucleotide positions 61 to 62, and wherein the sensitivity is at least 87% and specificity is at least 77%; or
d. the panel of one or more CpGs comprises at least the CpGs defined by SEQ ID NOs 1 to 1500 and identified at nucleotide positions 61 to 62, and wherein the sensitivity is at least 89% and specificity is at least 76%;
   preferably wherein the assay comprises determining methylation β-values for each CpG in the panel of one or more CpGs, more preferably wherein the cancer is cervical cancer.

The assay of the invention may be performed as above and additionally wherein when the cancer index value for the individual is about −0.167 or more, the individual is assessed as having CIN3 and/or cancer, or as having a high risk of CIN3 and/or cancer development, or wherein when the cancer index value for the individual is less than about −0.167, the individual is assessed as not having CIN3 and/or cancer or as having a low risk of CIN3 and/or cancer development, preferably wherein:
a. the panel of one or more CpGs comprises at least 500 of the CpGs identified at nucleotide positions 61 to 62 in SEQ ID NOs 1 to 5000, and wherein the sensitivity is at least 80% and the specificity is at least 61%;
b. the panel of one or more CpGs comprises at least the CpGs defined by SEQ ID NOs 1 to 500 and identified at nucleotide positions 61 to 62, and wherein the sensitivity is at least 77% and specificity is at least 90%;
c. the panel of one or more CpGs comprises at least the CpGs defined by SEQ ID NOs 1 to 1000 and identified at nucleotide positions 61 to 62, and wherein the sensitivity is at least 72% and specificity is at least 89%; or
d. the panel of one or more CpGs comprises at least the CpGs defined by SEQ ID NOs 1 to 1500 and identified at nucleotide positions 61 to 62, and wherein the sensitivity is at least 78% and specificity is at least 91%;
preferably wherein the assay comprises determining methylation β-values for each CpG in the panel of one or more CpGs, more preferably wherein the cancer is cervical cancer.

The assay of the invention may be performed as above and additionally wherein the step of determining the methylation status of a panel of one or more CpGs comprises determining the methylation status of one or more CpGs denoted by CG identified in a panel of one or more DMRs defined by SEQ ID NOs 5001 to 5418, optionally wherein the panel of one or more CpGs comprises two or more CpGs denoted by CG identified in the panel of DMR(s), three or more CpGs denoted by CG identified in the panel of DMR(s), four or more CpGs denoted by CG identified in the panel of DMR(s), or all CpGs denoted by CG identified in the DMR(s) defined by SEQ ID NOs 5001 to 5418.

The assay of the invention may be performed as above and additionally wherein the step of determining the methylation status of a panel of the one or more CpGs comprises determining the methylation status of five or more, six or more, seven or more, eight or more, or nine or more, or all of the CpGs denoted by CG within any one or more of the DMRs defined by SEQ ID NOs 5001 to 5418.

The assay of the invention may be performed as above and additionally wherein the step of determining the methylation status of a panel of one or more CpGs comprises determining the methylation status of two or more, three or more, four or more, five or more, six or more, seven or more, eight or more, or nine or more, or all of the CpGs denoted by CG within:
1. any combination of two, three, four, five, six, seven, eight, or nine or more of DMRs 1 to 418;
2. any combination of ten, twenty, thirty, forty, fifty, sixty, seventy, eighty, or ninety or more of DMRs 1 to 418;
3. all 418 of DMRs 1 to 418;
4. one DMR defined by SEQ ID NO: 5001, two DMRs defined by SEQ ID NOs: 5001 to 5002, three DMRs defined by SEQ ID NOs: 5001 to 5003, four DMRs defined by SEQ ID NOs: 5001 to 5004, five DMRs defined by SEQ ID NOs: 5001 to 5005, six DMRs defined by SEQ ID NOs: 5001 to 5006, seven DMRs defined by SEQ ID NOs: 5001 to 5007, eight DMRs defined by SEQ ID NOs: 5001 to 5008, or nine DMRs defined by SEQ ID NOs: 5001 to 5009;
5. any combination of one or more DMRs defined by SEQ ID NO: 5391, SEQ ID NO: 5392, SEQ ID NO: 5393, SEQ ID NO: 5407 and SEQ ID NO: 5414, preferably within all of SEQ ID NO: 5391, SEQ ID NO: 5392, SEQ ID NO: 5393, SEQ ID NO: 5407 and SEQ ID NO: 5414;
6. any combination of one or more DMRs defined by SEQ ID NO: 5392, SEQ ID NO: 5393 and SEQ ID NO: 5407, preferably within all of SEQ ID NO: 5392, SEQ ID NO: 5393 and SEQ ID NO: 5407;
7. ten DMRs defined by SEQ ID NOs: 5001 to 5010, twenty DMRs defined by SEQ ID NOs: 5001 to 5020, thirty DMRs defined by SEQ ID NOs: 5001 to 5030, forty DMRs defined by SEQ ID NOs: 5001 to 5040, fifty DMRs defined by SEQ ID NOs: 5001 to 5050, sixty DMRs defined by SEQ ID NOs: 5001 to 5060, seventy DMRs defined by SEQ ID NOs: 5001 to 5070, eighty DMRs defined by SEQ ID NOs: 5001 to 5080, or ninety DMRs defined by SEQ ID NOs: 5001 to 5090;
8. fifty DMRs defined by SEQ ID NOs: 5001 to 5050, SEQ ID NOs: 5051 to 5100, SEQ ID NOs: 5101 to 5150, SEQ ID NOs: 5151 to 5200, SEQ ID NOs: 5201 to 5250, SEQ ID NOs: 5301 to 5350, or SEQ ID NOs: 5341 to 5418; or
9. eighty one DMRs defined by SEQ ID NOs: 5015, 5016, 5017, 5025, 5026, 5027, 5028, 5029, 5032, 5033, 5048, 5049, 5050, 5053, 5054, 5057, 5068, 5069, 5071, 5072, 5073, 5074, 5075, 5076, 5077, 5083, 5090, 5091, 5092, 5093, 5094, 5095, 5096, 5099, 5102, 5137, 5138, 5140, 5143, 5146, 5147, 5148, 5149, 5150, 5151, 5164, 5165, 5167, 5175, 5176, 5177, 5179, 5180, 5185, 5204, 5224, 5226, 5228, 5232, 5246, 5248, 5285, 5287, 5293, 5307, 5309, 5315, 5317, 5324, 5328, 5337, 5339, 5340, 5348, 5349, 5354, 5361, 5362, 5366, 5368 and 5377.

The assay of the invention may be performed as above and additionally wherein the step of determining the methylation status of a panel of one or more CpGs comprises determining the methylation status of one or more CpGs within any one or more DMRs selected from the group of DMRs consisting of DMRs 1 to 418 as defined by SEQ ID NOs 5001 to 5418, including:
1. one or more CpGs within DMR 1 as defined by SEQ ID NO: 5001 and denoted by CG, preferably wherein the assay is characterised as having an ROC AUC of at least 0.894, and more preferably wherein the panel of one or more CpGs comprises at least the CpGs denoted by [[CG]];
2. one or more CpGs within DMR 2 as defined by SEQ ID NO: 5002 and denoted by CG, preferably wherein the assay is characterised as having an ROC AUC of at least 0.894, and more preferably wherein the panel of one or more CpGs comprises at least the CpGs denoted by [[CG]];
3. one or more CpGs within DMR 3 as defined by SEQ ID NO: 5003 and denoted by CG, preferably wherein the assay is characterised as having an ROC AUC of at least 0.894, and more preferably wherein the panel of one or more CpGs comprises at least the CpGs denoted by [[CG]];
4. one or more CpGs within DMR 4 as defined by SEQ ID NO: 5004 and denoted by CG, preferably wherein the assay is characterised as having an ROC AUC of at least 0.894, and more preferably wherein the panel of one or more CpGs comprises at least the CpGs denoted by [[CG]];
5. one or more CpGs within DMR 5 as defined by SEQ ID NO: 5005 and denoted by CG, preferably wherein the assay is characterised as having an ROC AUC of at least 0.894, and more preferably wherein the panel of one or more CpGs comprises at least the CpGs denoted by [[CG]];
6. one or more CpGs within DMR 6 as defined by SEQ ID NO: 5006 and denoted by CG, preferably wherein the assay is characterised as having an ROC AUC of at least 0.876, and more preferably wherein the panel of one or more CpGs comprises at least the CpGs denoted by [[CG]];
7. one or more CpGs within DMR 7 as defined by SEQ ID NO: 5007 and denoted by CG, preferably wherein the assay is characterised as having an ROC AUC of at least 0.874, and more preferably wherein the panel of one or more CpGs comprises at least the CpGs denoted by [[CG]];
8. one or more CpGs within DMR 8 as defined by SEQ ID NO: 5008 and denoted by CG, preferably wherein the assay is characterised as having an ROC AUC of at least 0.874, and more preferably wherein the panel of one or more CpGs comprises at least the CpGs denoted by [[CG]];
9. one or more CpGs within DMR 9 as defined by SEQ ID NO: 5009 and denoted by CG, preferably wherein the assay is characterised as having an ROC AUC of at least 0.874, and more preferably wherein the panel of one or more CpGs comprises at least the CpGs denoted by [[CG]]; and/or
10. one or more CpGs within DMR 10 as defined by SEQ ID NO: 5010 and denoted by CG, preferably wherein the assay is characterised as having an ROC AUC of at least 0.874, and more preferably wherein the panel of one or more CpGs comprises at least the CpGs denoted by [[CG]].

The assay of the invention may be performed as above and additionally wherein:
1. when the cancer index for the individual is about 0.016 or more the individual is classified as having CIN3 and/or cancer or as having a high risk of CIN3 and/or cancer development; or
2. when the cancer index for the individual is less than about 0.016 the individual is classified as not having CIN3 and/or cancer;
preferably wherein the assay has a specificity of 75% or more, more preferably wherein the assay comprises determining mean β-values for each CpG in the panel of one or more CpGs.

The assay of the invention may be performed as above and additionally wherein:
1. CpGs denoted by CG whose cancer index value is determined are located within at least DMR 1 defined by SEQ ID NO: 5001, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpGs from DMR 1, and more preferably wherein the cancer index value is the mean β-value for the CpGs denoted by [[CG]] in SEQ ID NO: 5001;
2. CpGs denoted by CG whose cancer index value is determined are located within at least DMR 1 defined by SEQ ID NO: 5001, and wherein when the cancer index for the individual is less than about 0.023 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least ten CpGs from DMR 1, and more preferably wherein the cancer index value is the mean β-value for the CpGs denoted by [[CG]] in SEQ ID NO: 5001;
3. CpGs denoted by CG whose cancer index value is determined are located within at least DMR 2 defined by SEQ ID NO: 5002, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 2, and more preferably wherein the cancer index value is the mean β-value for the CpGs denoted by [[CG]] in SEQ ID NO: 5002;
4. CpGs denoted by CG whose cancer index value is determined are located within at least DMR 2 defined by SEQ ID NO: 5002, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpGs from DMR 2, and more preferably wherein the cancer index value is the mean β-value for the CpGs denoted by [[CG]] in SEQ ID NO: 5002;
5. CpGs denoted by CG whose cancer index value is determined are located within at least DMR 3 defined by SEQ ID NO: 5003, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 3, and more preferably wherein the cancer index value is the mean β-value for the CpGs denoted by [[CG]] in SEQ ID NO: 5003;
6. CpGs denoted by CG whose cancer index value is determined are located within at least DMR 3 defined by SEQ ID NO: 5003, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 3, and more preferably wherein the cancer index value is the mean β-value for the CpGs denoted by [[CG]] in SEQ ID NO: 5003;
7. CpGs denoted by CG whose cancer index value is determined are located within at least DMR 4 defined by SEQ ID NO: 5004, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 4, and more preferably wherein the cancer index value is the mean β-value for the CpGs denoted by [[CG]] in SEQ ID NO: 5004;
8. CpGs denoted by CG whose cancer index value is determined are located within at least DMR 4 defined by SEQ ID NO: 5004, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 4, and more preferably wherein the cancer index value is the mean β-value for the CpGs denoted by [[CG]] in SEQ ID NO: 5004;
9. CpGs denoted by CG whose cancer index value is determined are located within at least DMR 5 defined by SEQ ID NO: 5005, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 5, and more preferably wherein the cancer index value is the mean β-value for the CpGs denoted by [[CG]] in SEQ ID NO: 5005; and/or
10. CpGs denoted by CG whose cancer index value is determined are located within at least DMR 5 defined by SEQ ID NO: 5005, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 5, and more preferably wherein the cancer index value is the mean β-value for the CpGs denoted by [[CG]] in SEQ ID NO: 5005.

The assay of the invention may be performed as above and additionally wherein the step of determining the methylation status of the one or more CpGs in the panel further comprises or additionally comprises determining the methylation status of each CpG within one or more of the sequences identified by SEQ ID NOs 5703 to 5786.

The assay of the invention may be performed as above and additionally wherein the step of determining the methylation status of the one or more CpGs in the panel comprises determining each CpG within:
1. SEQ ID NO 5759 and/or SEQ ID NO 5703 and/or SEQ ID NO 5731, and wherein when the cancer index value is about 1.146 or more the individual is classified as having CIN3 and/or cervical cancer or as having a high risk of CIN3 and/or cervical cancer development, and wherein the sensitivity of the assay is at least 95.00%, the specificity of the assay is at least 75.00%, and the AUC is 0.96, preferably wherein the cancer index value is the percent methylated reference for sequence defined by SEQ ID NO 5759 and/or SEQ ID NO 5703 and/or SEQ ID NO 5731;
2. SEQ ID NO 5759 and/or SEQ ID NO 5703 and/or SEQ ID NO 5731, and wherein when the cancer index value is less than about 1.146 the individual is classified as not having CIN3 and/or cervical cancer or as having a low risk of CIN3 and/or cervical cancer development, and wherein the sensitivity of the assay is at least 95.00%, the specificity of the assay is at least 75.00%, and the AUC is 0.96, preferably wherein the cancer index value is the percent methylated reference for sequence defined by SEQ ID NO 5759 and/or SEQ ID NO 5703 and/or SEQ ID NO 5731;
3. SEQ ID NO 5760 and/or SEQ ID NO 5704 and/or SEQ ID NO 5732, and wherein when the cancer index value is about 0.473 or more the individual is classified as having CIN3 and/or cervical cancer or as having a high risk of CIN3 and/or cervical cancer development, and wherein the sensitivity of the assay is at least 75.00%, the specificity of the assay is at least 85.00%, and the AUC is 0.96, preferably wherein the cancer index value is the percent methylated reference for sequence defined by SEQ ID NO 5760 and/or SEQ ID NO 5704 and/or SEQ ID NO 5732;
4. SEQ ID NO 5760 and/or SEQ ID NO 5704 and/or SEQ ID NO 5732, and wherein when the cancer index value is less than about 0.473 the individual is classified as not having CIN3 and/or cervical cancer or as having a low risk of CIN3 and/or cervical cancer development, and wherein the sensitivity of the assay is at least 75.00%, the specificity of the assay is at least 85.00%, and the AUC is 0.96, preferably wherein the cancer index value is the percent methylated reference for sequence defined by SEQ ID NO 5760 and/or SEQ ID NO 5704 and/or SEQ ID NO 5732;
5. SEQ ID NO 5761 and/or SEQ ID NO 5705 and/or SEQ ID NO 5733, and wherein when the cancer index value is about 1.546 or more the individual is classified as having CIN3 and/or cervical cancer or as having a high risk of CIN3 and/or cervical cancer development, and wherein the sensitivity of the assay is at least 95.00%, the specificity of the assay is at least 75.00%, and the AUC is 0.95, preferably wherein the cancer index value is the percent methylated reference for sequence defined by SEQ ID NO 5761;
6. SEQ ID NO 5761 and/or SEQ ID NO 5705 and/or SEQ ID NO 5733, and wherein when the cancer index value is less than about 1.546 the individual is classified as not having CIN3 and/or cervical cancer or as having a low risk of CIN3 and/or cervical cancer development, and wherein the sensitivity of the assay is at least 95.00%, the specificity of the assay is at least 75.00%, and the AUC is 0.95, preferably wherein the cancer index value is the percent methylated reference for sequence defined by SEQ ID NO 5761 and/or SEQ ID NO 5705 and/or SEQ ID NO 5733;
7. SEQ ID NO 5762 and/or SEQ ID NO 5706 and/or SEQ ID NO 5734, and wherein when the cancer index value is about 1.645 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5762 and/or SEQ ID NO 5706 and/or SEQ ID NO 5734;
8. SEQ ID NO 5762 and/or SEQ ID NO 5706 and/or SEQ ID NO 5734, and wherein when the cancer index value is less than about 1.645 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5762 and/or SEQ ID NO 5706 and/or SEQ ID NO 5734;
9. SEQ ID NO 5763 and/or SEQ ID NO 5707 and/or SEQ ID NO 5735, and wherein when the cancer index value is about 2.824 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.94, preferably wherein the cancer index value is the percent methylated reference for sequence defined by SEQ ID NO 5763 and/or SEQ ID NO 5707 and/or SEQ ID NO 5735; and/or
10. SEQ ID NO 5763 and/or SEQ ID NO 5707 and/or SEQ ID NO 5735, and wherein when the cancer index value is less than about 2.824 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.94, preferably wherein the cancer index value is the percent methylated reference for sequence defined by SEQ ID NO 5763 and/or SEQ ID NO 5707 and/or SEQ ID NO 5735.

The assay of the invention may be performed as above and additionally wherein the step of determining the methylation status of the one or more CpGs in the panel further comprises or additionally comprises determining the methylation status of each CpG within one or more of the sequences identified by SEQ ID NOs 5703, 5731, 5759, 5723, 5751, 5779, 5713, 5741, 5769, 5706, 5734, 5762, 5705, 5733, and 5761, or even more preferably wherein the panel of one or more CpGs further comprises or additionally comprises determining the methylation status of each CpG within one or more of the sequences identified by SEQ ID NOs 5703, 5731, 5759, 5713, 5741, 5769, 5706, 5734, and 5762.

The assay of the invention may be performed as above and additionally wherein the step of determining in the population of DNA molecules in the sample the methylation status of each CpG in the panel of one or more CpGs comprises:
1. performing a sequencing step to determine the sequence of each CpG;
2. hybridising DNA to an array comprising probes capable of discriminating between methylated and non-methylated forms of the CpGs and applying a detection system to the array so as to determine the methylation status of each CpG; and/or
3. performing a PCR step using methylation-specific primers, wherein the methylation status of the CpG is determined by the presence or absence of a PCR product.

The assay of the invention may be performed as above and additionally wherein the step of determining the methylation status of each CpG in the panel of one or more CpGs comprises:
1. bisulphite converting the DNA; or
2. performing the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The invention also provides a method of treating or preventing CIN3 and/or cervical cancer in an individual, the method comprising:
1. assessing the CIN3 and/or cervical cancer status of the individual by assessing the presence, absence or development of cancer in the individual by performing the assay of any one of the assays of the invention;
2. administering one or more therapeutic or preventative treatments to the individual based on the assessment.

The method of the invention may be performed as above and additionally wherein the individual is assessed as not having CIN3 and/or cancer or as having a low risk of CIN3 and/or cancer development, and wherein the cancer index value is about −0.530 or more and is less than about −0.330, and preferably wherein the assay comprises determining methylation β-values for each CpG in the panel of one or more CpGs, the individual is subjected to one or more treatments according to their cancer index value, the one or more treatments comprise a repeat assay according to any one of the assays of the invention, preferably wherein the repeat assay is performed about one year after the previous assay.

The assay of the invention may be performed as above and additionally wherein the individual is assessed as having a moderate risk of having CIN3 and/or cancer or as having a moderate risk of CIN3 and/or cancer development, and wherein the cancer index value is about −0.330 or more and is less than about −0.170, and preferably wherein the assay comprises determining methylation β-values for each CpG in the panel of one or more CpGs, the individual is subjected to one or more treatments according to their cancer index value, the one or more treatments comprise a test for human papilloma virus (HPV) status and wherein:
1. when the individual is HPV positive, a colposcopy, and optionally a transvaginal ultrasound and/or an endometrial biopsy to assess endometrium; or
2. when the individual is HPV negative, a repeat assay according to the assays of the invention, preferably wherein the repeat assay is performed about one year after the previous assay.

The assay of the invention may be performed as above and additionally wherein the individual is assessed as having CIN3 and/or cancer or as having a high risk of CIN3 and/or cancer development, and wherein the cancer index value is about −0.170 or more, and preferably wherein the assay comprises determining methylation β-values for each CpG in the panel of one or more CpGs, the individual is subjected to one or more treatments according to their cancer index value, the one or more treatments comprise a colposcopy, and wherein the colposcopy is negative, an endometrial biopsy and hysteroscopy.

The assay of the invention may be performed as above and additionally wherein the individual is assessed as having CIN3 and/or cancer or as having a high risk of CIN3 and/or cancer development, and wherein the cancer index value is about:
1. 5.000 or more, preferably wherein the step of determining the methylation status of the one or more CpGs in the panel comprises determining each CpG within SEQ ID NO 5768;
2. 1.000 or more, preferably wherein the step of determining the methylation status of the one or more CpGs in the panel comprises determining each CpG within SEQ ID NO 5765;
3. 2.000 or more, preferably wherein the step of determining the methylation status of the one or more CpGs in the panel comprises determining each CpG within SEQ ID NO 5759;
4. 5.000 or more, preferably wherein the step of determining the methylation status of the one or more CpGs in the panel comprises determining each CpG within SEQ ID NO 5785;
5. 4.000 or more, preferably wherein the step of determining the methylation status of the one or more CpGs in the panel comprises determining each CpG within SEQ ID NO 5763;
6. 4.000 or more, preferably wherein the step of determining the methylation status of the one or more CpGs in the panel comprises determining each CpG within SEQ ID NO 5781;
7. 1.000 or more, preferably wherein the step of determining the methylation status of the one or more CpGs in the panel comprises determining each CpG within SEQ ID NO 5769;
8. 1.000 or more, preferably wherein the step of determining the methylation status of the one or more CpGs in the panel comprises determining each CpG within SEQ ID NO 5766;
9. 4.000 or more, preferably wherein the step of determining the methylation status of the one or more CpGs in the panel comprises determining each CpG within SEQ ID NO 5761;
10. 4.000 or more, preferably wherein the step of determining the methylation status of the one or more CpGs in the panel comprises determining each CpG within SEQ ID NO 5779;
11. 4.000 or more, preferably wherein the step of determining the methylation status of the one or more CpGs in the panel comprises determining each CpG within SEQ ID NO 5764;
12. 5.000 or more, preferably wherein the step of determining the methylation status of the one or more CpGs in the panel comprises determining each CpG within SEQ ID NO 5772;
13. 2.000 or more, preferably wherein the step of determining the methylation status of the one or more CpGs in the panel comprises determining each CpG within SEQ ID NO 5773;
14. 1.000 or more, preferably wherein the step of determining the methylation status of the one or more CpGs in the panel comprises determining each CpG within SEQ ID NO 5778;
15. 5.000 or more, preferably wherein the step of determining the methylation status of the one or more CpGs in the panel comprises determining each CpG within SEQ ID NO 5767;
16. 5.000 or more, preferably wherein the step of determining the methylation status of the one or more CpGs in the panel comprises determining each CpG within SEQ ID NO 5774;
17. more than 0.000, preferably wherein the step of determining the methylation status of the one or more CpGs in the panel comprises determining each CpG within SEQ ID NO 5760;
18. 4.000 or more, preferably wherein the step of determining the methylation status of the one or more CpGs in the panel comprises determining each CpG within SEQ ID NO 5771;
19. 2.000 or more, preferably wherein the step of determining the methylation status of the one or more CpGs in the panel comprises determining each CpG within SEQ ID NO 5762;
20. 7.000 or more, preferably wherein the step of determining the methylation status of the one or more CpGs in the panel comprises determining each CpG within SEQ ID NO 5780;
21. 6.000 or more, preferably wherein the step of determining the methylation status of the one or more CpGs in the panel comprises determining each CpG within SEQ ID NO 5776;
22. 5.000 or more, preferably wherein the step of determining the methylation status of the one or more CpGs in the panel comprises determining each CpG within SEQ ID NO 5783;
23. 6.000 or more, preferably wherein the step of determining the methylation status of the one or more CpGs in the panel comprises determining each CpG within SEQ ID NO 5782;
24. 6.000 or more, preferably wherein the step of determining the methylation status of the one or more CpGs in the panel comprises determining each CpG within SEQ ID NO 5784;
25. 5.000 or more, preferably wherein the step of determining the methylation status of the one or more CpGs in the panel comprises determining each CpG within SEQ ID NO 5777;
26. 2.000 or more, preferably wherein the step of determining the methylation status of the one or more CpGs in the panel comprises determining each CpG within SEQ ID NO 5786;
27. 7.000 or more, preferably wherein the step of determining the methylation status of the one or more CpGs in the panel comprises determining each CpG within SEQ ID NO 5775; or
28. 3.000 or more, preferably wherein the step of determining the methylation status of the one or more CpGs in the panel comprises determining each CpG within SEQ ID NO 5770,
and more preferably wherein the assay comprises determining percent methylated reference for the sequence defined by the specified SEQ ID NO, the individual is subjected to one or more treatments according to their cancer index value, the one or more treatments comprise a colposcopy, and wherein the colposcopy is negative, an endometrial biopsy, and wherein the endometrial biopsy is also negative, a repeat assay according to the assays of the invention, preferably wherein the repeat assay is performed about one year after the previous assay.

The assay of the invention may be performed as above and additionally wherein the one or more treatments that the individual is subjected to are repeated on a monthly, three monthly, six monthly, yearly or two yearly basis following an initial administration.

The invention also provides a method of monitoring the CIN3 and/or cancer status of an individual according to the individual's cancer index value, the method comprising: (a) assessing the presence, absence or development of CIN3 and/or cancer in an individual by performing the assay according to any one of the assays of the invention at a first time point; (b) assessing the presence, absence or development of CIN3 and/or cancer in the individual by performing the assay according to any one of the assays of the invention at one or more further time points; and (c) monitoring any change in cancer index value and/or the CIN3 and/or cancer status of the individual between time points.

The method of the invention may be performed as above and additionally wherein the further time points are monthly, three monthly, six monthly, yearly or two yearly basis following an initial assessment.

The method of the invention may be performed as above and additionally wherein depending on the cancer status of the individual, one or more treatments are administered to the individual according to any one of the methods of the invention, or when the cancer index value of the individual is:
1. less than about −0.530, and preferably wherein the assay comprises determining methylation β-values for each CpG in the panel of one or more CpGs, no treatment is administered to the individual;
2. less than about 5.000, and preferably wherein the step of determining the methylation status of the one or more CpGs in the panel comprises determining each CpG within 5768, and more preferably wherein the assay comprises determining percent methylated reference for the sequence defined by the specified SEQ ID NO, no treatment is administered to the individual;
3. less than about 1.000, and preferably wherein the step of determining the methylation status of the one or more CpGs in the panel comprises determining each CpG within 5765, and more preferably wherein the assay comprises determining percent methylated reference for the sequence defined by the specified SEQ ID NO, no treatment is administered to the individual;
4. less than about 2.000, and preferably wherein the step of determining the methylation status of the one or more CpGs in the panel comprises determining each CpG within 5759, and more preferably wherein the assay comprises determining percent methylated reference for the sequence defined by the specified SEQ ID NO, no treatment is administered to the individual;
5. less than about 5.000, and preferably wherein the step of determining the methylation status of the one or more CpGs in the panel comprises determining each CpG within 5785, and more preferably wherein the assay comprises determining percent methylated reference for the sequence defined by the specified SEQ ID NO, no treatment is administered to the individual;
6. less than about 4.000, and preferably wherein the step of determining the methylation status of the one or more CpGs in the panel comprises determining each CpG within 5763, and more preferably wherein the assay comprises determining percent methylated reference for the sequence defined by the specified SEQ ID NO, no treatment is administered to the individual;
7. less than about 4.000, and preferably wherein the step of determining the methylation status of the one or more CpGs in the panel comprises determining each CpG within 5781, and more preferably wherein the assay comprises determining percent methylated reference for the sequence defined by the specified SEQ ID NO, no treatment is administered to the individual;
8. less than about 1.000, and preferably wherein the step of determining the methylation status of the one or more CpGs in the panel comprises determining each CpG within 5769, and more preferably wherein the assay comprises determining percent methylated reference for the sequence defined by the specified SEQ ID NO, no treatment is administered to the individual;
9. less than about 1.000, and preferably wherein the step of determining the methylation status of the one or more CpGs in the panel comprises determining each CpG within 5766, and more preferably wherein the assay comprises determining percent methylated reference for the sequence defined by the specified SEQ ID NO, no treatment is administered to the individual;
10. less than about 4.000, and preferably wherein the step of determining the methylation status of the one or more CpGs in the panel comprises determining each CpG within 5761, and more preferably wherein the assay comprises determining percent methylated reference for the sequence defined by the specified SEQ ID NO, no treatment is administered to the individual;
11. less than about 4.000, and preferably wherein the step of determining the methylation status of the one or more CpGs in the panel comprises determining each CpG within 5779, and more preferably wherein the assay comprises determining percent methylated reference for the sequence defined by the specified SEQ ID NO, no treatment is administered to the individual;
12. less than about 4.000, and preferably wherein the step of determining the methylation status of the one or more CpGs in the panel comprises determining each CpG within 5764, and more preferably wherein the assay comprises determining percent methylated reference for the sequence defined by the specified SEQ ID NO, no treatment is administered to the individual;
13. less than about 5.000, and preferably wherein the step of determining the methylation status of the one or more CpGs in the panel comprises determining each CpG within 5772, and more preferably wherein the assay comprises determining percent methylated reference for the sequence defined by the specified SEQ ID NO, no treatment is administered to the individual;
14. less than about 2.000, and preferably wherein the step of determining the methylation status of the one or more CpGs in the panel comprises determining each CpG within 5773, and more preferably wherein the assay comprises determining percent methylated reference for the sequence defined by the specified SEQ ID NO, no treatment is administered to the individual;
15. less than about 1.000, and preferably wherein the step of determining the methylation status of the one or more CpGs in the panel comprises determining each CpG within 5778, and more preferably wherein the assay comprises determining percent methylated reference for the sequence defined by the specified SEQ ID NO, no treatment is administered to the individual;
16. less than about 5.000, and preferably wherein the step of determining the methylation status of the one or more CpGs in the panel comprises determining each CpG within 5767, and more preferably wherein the assay comprises determining percent methylated reference for the sequence defined by the specified SEQ ID NO, no treatment is administered to the individual;
17. less than about 5.000, and preferably wherein the step of determining the methylation status of the one or more CpGs in the panel comprises determining each CpG within 5774, and more preferably wherein the assay comprises determining percent methylated reference for the sequence defined by the specified SEQ ID NO, no treatment is administered to the individual;
18. 0.000, preferably wherein the step of determining the methylation status of the one or more CpGs in the panel comprises determining each CpG within 5760, and more preferably wherein the assay comprises determining percent methylated reference for the sequence defined by the specified SEQ ID NO, no treatment is administered to the individual;
19. less than about 4.000, and preferably wherein the step of determining the methylation status of the one or more CpGs in the panel comprises determining each CpG within 5771, and more preferably wherein the assay comprises determining percent methylated reference for the sequence defined by the specified SEQ ID NO, no treatment is administered to the individual;
20. less than about 2.000, and preferably wherein the step of determining the methylation status of the one or more CpGs in the panel comprises determining each CpG within 5762, and more preferably wherein the assay comprises determining percent methylated reference for the sequence defined by the specified SEQ ID NO, no treatment is administered to the individual;
21. less than about 7.000, and preferably wherein the step of determining the methylation status of the one or more CpGs in the panel comprises determining each CpG within 5780, and more preferably wherein the assay comprises determining percent methylated reference for the sequence defined by the specified SEQ ID NO, no treatment is administered to the individual;
22. less than about 6.000, and preferably wherein the step of determining the methylation status of the one or more CpGs in the panel comprises determining each CpG within 5776, and more preferably wherein the assay comprises determining percent methylated reference for the sequence defined by the specified SEQ ID NO, no treatment is administered to the individual;
23. less than about 5.000, and preferably wherein the step of determining the methylation status of the one or more CpGs in the panel comprises determining each CpG within 5783, and more preferably wherein the assay comprises determining percent methylated reference for the sequence defined by the specified SEQ ID NO, no treatment is administered to the individual;
24. less than about 6.000, and preferably wherein the step of determining the methylation status of the one or more CpGs in the panel comprises determining each CpG within 5782, and more preferably wherein the assay comprises determining percent methylated reference for the sequence defined by the specified SEQ ID NO, no treatment is administered to the individual;
25. less than about 6.000, and preferably wherein the step of determining the methylation status of the one or more CpGs in the panel comprises determining each CpG within 5784, and more preferably wherein the assay comprises determining percent methylated reference for the sequence defined by the specified SEQ ID NO, no treatment is administered to the individual;
26. less than about 5.000, and preferably wherein the step of determining the methylation status of the one or more CpGs in the panel comprises determining each CpG within 5777, and more preferably wherein the assay comprises determining percent methylated reference for the sequence defined by the specified SEQ ID NO, no treatment is administered to the individual;
27. less than about 2.000, and preferably wherein the step of determining the methylation status of the one or more CpGs in the panel comprises determining each CpG within 5786, and more preferably wherein the assay comprises determining percent methylated reference for the sequence defined by the specified SEQ ID NO, no treatment is administered to the individual;
28. less than about 7.000, and preferably wherein the step of determining the methylation status of the one or more CpGs in the panel comprises determining each CpG within 5775, and more preferably wherein the assay comprises determining percent methylated reference for the sequence defined by the specified SEQ ID NO, no treatment is administered to the individual; or
29. less than about 3.000, and preferably wherein the step of determining the methylation status of the one or more CpGs in the panel comprises determining each CpG within 5770, and more preferably wherein the assay comprises determining percent methylated reference for the sequence defined by the specified SEQ ID NO, no treatment is administered to the individual.

The method of the invention may be performed as above and additionally wherein an increase in the cancer index value indicates a negative response to the one or more treatments.

The method of the invention may be performed as above and additionally wherein changes are made to the one or more treatments if a negative response is identified.

The method of the invention may be performed as above and additionally wherein a decrease in the cancer index value indicates a positive response to the one or more treatments.

The method of the invention may be performed as above and additionally wherein changes are made to the one or more treatments if a positive response is identified.

The assay of the invention may be performed as above and additionally wherein the sample is obtained from a tissue comprising epithelial cells, preferably wherein the sample is not obtained from ovarian or endometrial tissue.

The assay of the invention may be performed as above and additionally wherein the sample is obtained from:
1. cervical tissue;
2. vaginal tissue;
3. cervicovaginal tissue;
4. buccal tissue;
preferably wherein the sample is obtained from cervical tissue, most preferably wherein the sample is obtained from tissue from a cervical smear.

The assay of the invention may be performed as above and additionally wherein the assay is for assessing the presence, absence or development of:
1. grade 3 cervical epithelial neoplasia (CIN3) and/or cervical cancer, particularly wherein the cervical cancer is squamous cell cancer, an adenocarcinoma or an adenosquamous carcinoma;
2. endometrial cancer, preferably wherein the endometrial cancer is an endometriod cancer, uterine carcinosarcoma, squamous cell carcinoma, small cell carcinoma, transitional carcinoma, serous carcinoma, clear-cell carcinoma, mucinous adenocarcinoma, undifferentiated carcinoma, dedifferentiated carcinoma or serous adenocarcinoma.

The invention also provides an array capable of discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 5000 and identified at nucleotide positions 61 to 62, and identified in SEQ ID NOs 5001 to 5418 and denoted by CG.

The array of the invention may be performed as above and additionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less.

The array of the invention may be performed as above and additionally wherein the panel comprises any panel of CpGs defined in the assays of any one of the assays of the invention.

The array of the invention may be performed as above and additionally further comprising one or more oligonucleotides comprising any set of CpGs defined in the assays of any one of the assays of the invention, wherein the one or more oligonucleotides are hybridized to corresponding oligonucleotide probes of the array.

The invention also provides a hybridized array, wherein the array is obtainable by hybridizing to an array according to any one of the arrays of the invention a group of oligonucleotides comprising any panel of CpGs defined in the assays of any one of the assays of the invention.

The invention also provides a process for making the hybridized array according to the hybridised array of the invention, comprising contacting an array according to any one of the arrays of the invention with a group of oligonucleotides comprising any panel of CpGs defined in any one of the assays of the invention.

### BRIEF DESCRIPTION OF THE FIGURES

FIG. 1 shows the experimental design underpinning the discovery and validation of the WID-CIN-index.

FIG. 2 shows (A) distribution of the WID-CIN-index in the diagnostic validation set with cutoffs corresponding to 90% (dashed) and 75% specificity (dotted). (B) receiver operating characteristic (ROC) curve corresponding to the diagnostic validation set with separate curves for women >30 years and ≤30 years of age. (C) distribution of the WID-CIN-index in CIN1 and CIN2 cases as part of the diagnostic validation set with 90% (dashed) and 75% (dotted) specificity cutoffs.

FIG. 3 shows (A) dependence of the WID-CIN-index in HPV+ control samples on biobank storage time. (B) the WID-CIN-index in the predictive validation set consisting of HPV-positive and cytology-negative samples taken 1-4 years prior to either a diagnosis with CIN3+(red points) or censoring (blue points). (C) ROC curve corresponding to the predictive validation set.

FIG. 4 shows (A) distribution of the WID-CIN-index in the endometrial diagnostic validation set consisting of samples from healthy controls and women with endometrial cancer. (B) ROC curve corresponding to the endometrial diagnostic validation set.

FIG. 5 shows CIN3+ true positive rate (TPR) and CIN1/2 true negative rate (TNR) corresponding to a) 100% sensitivity to detect CIN3+ cases in all cytology positive samples and b) 75% sensitivity to detect CIN3+ cases in all HPV+ samples. Based on the diagnostic validation datasets. Separate cutoffs for women <30 and ≥30 years of age are included.

FIG. 6 shows (A) cell-type composition in the combined discovery and diagnostic validation sets as determined by the hEpiDISH algorithm. (B) distribution of pvalues after comparing HPV+ controls to CIN3+ cases in the discovery set (based on a linear regression model with adjustment for age and immunce cell proportion). (C) distribution of the estimated epithelial and immune delta-betas. (D) performance of ridge and lasso classifiers based on out-of-bag estimates from 10-fold cross validation on the discovery set. (E) odds ratios when comparing the genomic annotation of the 5,000 CpGs comprising the WID-CIN-index to the 777,005 CpGs that were used in the analysis.

FIG. 7 shows (A) dependence of the WID-CIN-index on immune cell proportion in the HPV+ controls and CIN3+ cases from the discovery set. (B) dependence of the WID-CIN-index on age in the HPV+ controls and CIN3+ cases from the discovery set. (C) ROC curve corresponding to the performance of the WID-CIN-index in HPV− controls and CIN3+ cases from the discovery set.

FIG. 8 shows (A) the cell-type composition of samples from the predictive validation set based on the hEpiDISH algorithm. (B) dependence of the WID-CIN-index on age in the HPV+ controls and CIN3+ cases from the predictive validation set.

FIG. 9 shows (A) the island subcomponent of the WID-CIN-index as a function of immune cell proportion in the diagnostic validation set. (B) ROC curve corresponding to the island subcomponent of the WID-CIN-index in the diagnostic validation set. (C) the island subcomponent of the WID-CIN-index as a function of immune cell proportion in the predictive validation set. (D) ROC curve corresponding to the island subcomponent of the WID-CIN-index in the predictive validation set. (E) the open-sea subcomponent of the WID-CIN-index as a function of immune cell proportion in the diagnostic validation set. (F) ROC curve corresponding to the open-sea subcomponent of the WID-CIN-index in the diagnostic validation set. (G) the open-sea subcomponent of the WID-CIN-index as a function of immune cell proportion in the predictive validation set. (H) ROC curve corresponding to the open-sea subcomponent of the WID-CIN-index in the predictive validation set.

FIG. 10 shows (A) the cell-type composition of samples from the endometrial diagnostic validation set based on the hEpiDISH algorithm. (B) dependence of the WID-CIN-index on age in the HPV+ controls and CIN3+ cases from the endometrial diagnostic validation set.

FIG. 11 shows cutpoints applied to the patient data, and consequent specificity and sensitivity for CIN3 status discrimination achieved when these cutpoints are applied.

### DETAILED DESCRIPTION OF THE INVENTION

### Identification of CpGs

The present inventors sought to identify CpG methylation-based assays capable of assessing the presence, absence or development of CIN3 and/or cancer, particularly cervical or endometrial cancer, most preferably cervical cancer, in an individual. Any of the assays described herein for assessing the presence, absence or development of CIN3 and/or cancer, particularly cervical or endometrial cancer, most preferably cervical cancer, in an individual are capable of being utilised for assessing the presence, absence or development of cervical cancer and/or endometrial cancer, particularly cervical cancer. The present inventors compared CpG methylation levels in non-cancerous epithelial cells, particularly be derived from the cervix, the vagina, the buccal area, blood and/or urine, preferably derived from a cervical liquid-based cytology sample, and more preferably a cervical smear sample across groups of women that were either known to be both cervical and endometrial cancer negative, or known to be cervical and/or endometrial cancer positive. This led to the surprising establishment of a “cancer index”, used interchangeable herein with “index”, “index value”, “WID-CIN-Index” or “WID-Index” (WID=women's risk identification).

A CpG as defined herein refers to the CG dinucleotide motif identified in relation to each SEQ ID NO., wherein the CG dinucleotide of interest is denoted by CG and by [[CG]]. Thus by determining the methylation status of any panel of one or more CpGs defined by or identified in a given SEQ ID NO, it is meant that a determination is made as to the methylation status of the cytosine of the CG dinucleotide motif identified in square brackets in the panel of one or more CpGs in each sequence shown below, accepting that variations in the sequence upstream and downstream of any given CpG may exist due to sequencing errors or variation between individuals.

As set out in more detail in the Examples, the methylation status of sub-selections of the 5000 CpGs, as identified in SEQ ID NOs 1 to 5000, may be determined in order to assess an individual for the presence, absence or development of CIN3 and/or cancer, particularly cervical or endometrial cancer, most preferably cervical cancer, with high sensitivity and specificity. A panel of one or more of the CpGs identified in SEQ ID NOs 1 to 5000 may be utilised to derive a cancer index for an individual in accordance with the invention described herein.

The methylation status of a panel of one or more CpGs of the 5000 CpGs defined according to SEQ ID NOs: 1 to 5000 may be assessed by any suitable technique. As explained in more detail in the Examples below, one particular exemplary technique which the inventors have used is an array-based analysis technique coupled with beta value analysis. SEQ ID NOs 1 to 5000 correspond to the sequences of commercial probes utilised in said array.

The inventors further identified 418 differentially methylated regions (DMRs) with relevance to CIN3 and cancer, particularly cervical or endometrial cancer. The nucleotide sequences of the 418 DMRs are defined respectively by the nucleotide sequences of SEQ ID NO: 5001 to 5418 as set out in Table 1 below, accepting that variation in the nucleotide sequence of any given DMR may exist due to sequencing errors and/or variation between individuals. In each of the sequences corresponding to SEQ ID NO: 5001 to 5418, the cytosine of the CG dinucleotide motif identified in square brackets or double square brackets is a cytosine of a CpG which may be included in a panel of CpGs when performing the assays of the invention.

The inventors further defined 28 regions within a select number of the 418 DMRs with particular relevance to CIN3 and cancer, particularly cervical or endometrial cancer. The nucleotide sequences of the 28 regions are defined respectively by the nucleotide sequences of SEQ ID NOs: 5703 to 5786 as set out in Table 2 below, accepting that variation in the nucleotide sequence of any given DMR may exist due to sequencing errors and/or variation between individuals. When any one or more of the 28 regions are included in a panel of CpGs when performing the assays of the invention, the methylation status of every cytosine within a CG dinucleotide in the region is determined. The amplicon sequences generated by the 28 primer and probe reactions as set out Table 2 are described and defined by SEQ ID NOs 5787 to 5814 and in Table 12. In any of the assays described herein, the step of determining the methylation status of a panel of one or more CpGs may comprise determining the methylation status of one or more CpGs within any one or more the amplicons defined by SEQ ID NOs 5787 to 5814 and denoted by CG. More preferably, in any of the assays described herein, the step of determining the methylation status of a panel of one or more CpGs may comprise determining the methylation status of one or more CpGs within any one or more the amplicons defined by SEQ ID NOs 5787, 5790, 5797, 5807 and 5789, although more preferably 5787, 5790, 5797, and denoted by CG. Yet more preferably, in any of the assays described herein, the step of determining the methylation status of a panel of one or more CpGs may comprise determining the methylation status of all of the CpGs denoted by CG in the amplicons defined by SEQ ID NOs 5787, 5790, 5797, 5807 and 5789, although more preferably 5787, 5790, 5797.

TABLE 1
The nucleotide sequences of the 418 DMRs are
defined respectively by the nucleotide
sequences of SEQ ID NO: 5001 to 5418.

SEQ ID NO: 5001
C[CG]T[CG]C[CG]CT[CG]CCCTTGGC[CG][CG]CCAGC[CG
]CAGGGTCCT[CG]G[CG]CC[CG]GGGG[CG]GCAGCAGGTTGG
AG[CG]CAC[CG]TG[CG][CG]TCCTGCCCTCT[CG]G[CG]CC
TC[CG]C[CG]TCC[CG]GAGGCC[CG]GGCTCCT[CG]GTCCC[
CG]C[CG]GCTCC[CG]CTCCATGGC[CG][CG]TGT[CG]C[CG
][CG]CC[CG]GCTCCTC[CG]C[CG]CTCA[CG]CC[CG]CC[C
G][CG]C[CG]GCCTCTTAGGTTATCATCACT[CG][CG]GCC[C
G]GG[CG][CG]G[CG]GTG[CG]AGCTCCC[CG]CCTG[CG]GG
A[CG]CA[CG]GAGAC[CG][CG]GTCAG[CG][CG]C[CG]CCT
GGC[[CG]]GCCCAG[CG][CG]CCCAGCC[CG][CG]CCCAGCC
C[CG]TCCACTCC[CG]TCCAGCCC[CG]C[CG]CC[CG]GC[CG
]CAGTCCCCAGGG[CG]CAAGCTGGG[CG]GGTGC[CG]CACAGA
GCCCCCTTGTCCT[CG]C[CG]CC[CG]CCCC[CG]GCC[CG]GG
T[CG]TTTC[CG]C[CG][CG]GTGGCCA[CG]GCCC[CG]CCCT
[CG]TTC[CG][CG]CC[CG]GACTGGGCCA[CG]C[CG]GATAG
[CG]GGAAACAAAAAAAGCC[CG]AGCTGGAAACTTCAGAGAGGT
TTAGTTT[[CG]]TTTCCCAGAAGCATCAGTT[[CG]]GTCCCAA
AA[[CG]]CTGCAAA[[CG]][CG][CG]CT
SEQ ID NO: 5002
T[CG]CCCTTGGC[CG][CG]CCAGC[CG]CAGGGTCCT[CG]G[
CG]CC[CG]GGGG[CG]GCAGCAGGTTGGAG[CG]CAC[CG]TG[
CG][CG]TCCTGCCCTCT[CG]G[CG]CCTC[CG]C[CG]TCC[C
G]GAGGCC[CG]GGCTCCT[CG]GTCCC[CG]C[CG]GCTCC[CG]
CTCCATGGC[CG][CG]TGT[CG]C[CG][CG]CC[CG]GCTCCT
C[CG]C[CG]CTCA[CG]CC[CG]CC[CG][CG]C[CG]GCCTCT
TAGGTTATCATCACT[CG][CG]GCC[CG]GG[CG][CG]G[CG]
GTG[CG]AGCTCCC[CG]CCTG[CG]GGA[CG]CA[CG]GAGAC[
CG][CG]GTCAG[CG][CG]C[CG]CCTGGC[CG]GCCCAG[[CG
]][[CG]]CCCAGCC[[CG]][[CG]]CCCAGCCC[CG]TCCACT
CC[CG]TCCAGCCC[CG]C[CG]CC[CG]GC[CG]CAGTCCCCAG
GG[CG]CAAGCTGGG[CG]GGTGC[CG]CACAGAGCCCCCTTGTC
CT[CG]C[CG]CC[CG]CCCC[CG]GCC[CG]GGT[CG]TTTC[C
G]C[CG][CG]GTGGCCA[CG]GCCC[CG]CCCT[CG]TTC[CG]
[CG]CC[CG]GACTGGGCCA[CG]C[CG]GATAG[CG]GGAAACA
AAAAAAGCC[CG]AGCTGGAAACTTCAGAGAGGTTTAGTTT[CG]
TTTCCCAGAAGCATCAGTT[[CG]]GTCCCAAAA[CG]CTGCAAA
[CG][CG][CG]CTGCCTGCAGTA
SEQ ID NO: 5003
GC[CG][CG]CCAGC[CG]CAGGGTCCT[CG]G[CG]CC[CG]GG
GG[CG]GCAGCAGGTTGGAG[CG]CAC[CG]TG[CG][CG]TCCT
GCCCTCT[CG]G[CG]CCTC[CG]C[CG]TCC[CG]GAGGCC[CG
]GGCTCCT[CG]GTCCC[CG]C[CG]GCTCC[CG]CTCCATGGC[
CG][CG]TGT[CG]C[CG][CG]CC[CG]GCTCCTC[CG]C[CG]
CTCA[CG]CC[CG]CC[CG][CG]C[CG]GCCTCTTAGGTTATCA
TCACT[CG][CG]GCC[CG]GG[CG][CG]G[CG]GTG[CG]AGC
TCCC[CG]CCTG[CG]GGA[CG]CA[CG]GAGAC[CG][CG]GTC
AG[CG][CG]C[CG]CCTGGC[CG]GCCCAG[CG][CG]CCCAGC
C[[CG]][[CG]]CCCAGCCC[CG]TCCACTCC[CG]TCCAGCCC
[CG]C[CG]CC[CG]GC[CG]CAGTCCCCAGGG[CG]CAAGCTGG
G[CG]GGTGC[CG]CACAGAGCCCCCTTGTCCT[CG]C[CG]CC[
CG]CCCC[CG]GCC[CG]GGT[CG]TTTC[CG]C[CG][CG]GTG
GCCA[CG]GCCC[CG]CCCT[CG]TTC[CG][CG]CC[CG]GACT
GGGCCA[CG]C[CG]GATAG[CG]GGAAACAAAAAAAGCC[CG]A
GCTGGAAACTTCAGAGAGGTTTAGTTT[[CG]]TTTCCCAGAAGC
ATCAGTT[[CG]]GTCCCAAAA[[CG]]CTGCAAA[CG][CG][C
G]CTGCCTGCAGTAGGAGAGAGG
SEQ ID NO: 5004
GCT[CG]CCCTTGGC[CG][CG]CCAGC[CG]CAGGGTCCT[CG]
G[CG]CC[CG]GGGG[CG]GCAGCAGGTTGGAG[CG]CAC[CG]T
G[CG][CG]TCCTGCCCTCT[CG]G[CG]CCTC[CG]C[CG]TCC
[CG]GAGGCC[CG]GGCTCCT[CG]GTCCC[CG]C[CG]GCTCC[
CG]CTCCATGGC[CG][CG]TGT[CG]C[CG][CG]CC[CG]GCT
CCTC[CG]C[CG]CTCA[CG]CC[CG]CC[CG][CG]C[CG]GCC
TCTTAGGTTATCATCACT[CG][CG]GCC[CG]GG[CG][CG]G[
CG]GTG[CG]AGCTCCC[CG]CCTG[CG]GGA[CG]CA[CG]GAG
AC[CG][CG]GTCAG[CG][CG]C[CG]CCTGGC[CG]GCCCAG[
[CG]][[CG]]CCCAGCC[CG][CG]CCCAGCCC[CG]TCCACTC
C[CG]TCCAGCCC[CG]C[CG]CC[CG]GC[CG]CAGTCCCCAGG
G[CG]CAAGCTGGG[CG]GGTGC[CG]CACAGAGCCCCCTTGTCC
T[CG]C[CG]CC[CG]CCCC[CG]GCC[CG]GGT[CG]TTTC[CG
]C[CG][CG]GTGGCCA[CG]GCCC[CG]CCCT[CG]TTC[CG][
CG]CC[CG]GACTGGGCCA[CG]C[CG]GATAG[CG]GGAAACAA
AAAAAGCC[CG]AGCTGGAAACTTCAGAGAGGTTTAGTTT[CG]T
TTCCCAGAAGCATCAGTT[CG]GTCCCAAAA[CG]CTGCAAA[CG
][CG][CG]CTGCCTGCAG
SEQ ID NO: 5005
[CG][CG]CCAGC[CG]CAGGGTCCT[CG]G[CG]CC[CG]GGGG
[CG]GCAGCAGGTTGGAG[CG]CAC[CG]TG[CG][CG]TCCTGC
CCTCT[CG]G[CG]CCTC[CG]C[CG]TCC[CG]GAGGCC[CG]G
GCTCCT[CG]GTCCC[CG]C[CG]GCTCC[CG]CTCCATGGC[CG
][CG]TGT[CG]C[CG][CG]CC[CG]GCTCCTC[CG]C[CG]CT
CA[CG]CC[CG]CC[CG][CG]C[CG]GCCTCTTAGGTTATCATC
ACT[CG][CG]GCC[CG]GG[CG][CG]G[CG]GTG[CG]AGCTC
CC[CG]CCTG[CG]GGA[CG]CA[CG]GAGAC[CG][CG]GTCAG
[CG][CG]C[CG]CCTGGC[CG]GCCCAG[CG][CG]CCCAGCC[
[CG]][[CG]]CCCAGCCC[CG]TCCACTCC[CG]TCCAGCCC[C
G]C[CG]CC[CG]GC[CG]CAGTCCCCAGGG[CG]CAAGCTGGG[
CG]GGTGC[CG]CACAGAGCCCCCTTGTCCT[CG]C[CG]CC[CG
]CCCC[CG]GCC[CG]GGT[CG]TTTC[CG]C[CG][CG]GTGGC
CA[CG]GCCC[CG]CCCT[CG]TTC[CG][CG]CC[CG]GACTGG
GCCA[CG]C[CG]GATAG[CG]GGAAACAAAAAAAGCC[[CG]]A
GCTGGAAACTTCAGAGAGGTTTAGTTT[CG]TTTCCCAGAAGCAT
CAGTT[[CG]]GTCCCAAAA[[CG]]CTGCAAA[[CG]][CG][C
G]CTGCCTGCAGTAGGAGAGAGGAA
SEQ ID NO: 5006
TTTTCACCATTTTACAGT[CG]AAGAAGGGAAGATAAGAGG[CG]
GTGAATC[CG]AGACTTTCATTCAGGTGA[CG]ATCCCT[CG][C
G]GGCACTCACTCT[CG]CTCACACTACAATCAGATCCTCCATG[
CG]GCTTTCATATACTCTGTCACACT[CG]CAGCCAGAATCCCCT
[CG]GATGGACTCTTGCAAACCTCCAGCCCCATCC[CG]GGATC[
CG]TG[CG]TCTTCTACAGCCACCACTTGGGCCAG[CG]TCCAGG
A[CG]CCCAAG[CG]TAACCTCCCCACTCTAAAC[CG]TTAGGAA
TCAGTGGCTGGACTCAC[CG][CG]TCATAAAAGCATAAACTGTG
ACC[CG]ATGAGTC[CG][CG]CACT[CG]GAAAACAAGAGAA[C
G]CTCCCA[CG]TCTC[CG]GG[CG]CAGCTTGTGCCAG[CG]AC
TACTAGGTCAGGGGG[CG]CAGCTGAGAGAATTCAAGCAACCTCC
C[CG]CAAC[CG]GTTC[CG]C[CG][CG]TTTGTGGGCTGGTAG
CC[CG]GAATACATTTCCCAGAGGCCTT[CG][CG]GC[[CG]]A
[CG]TGCTT[CG][CG]CAGGAA[CG]CAGC[CG]CCTCC[CG]A
CTGGAGGA[CG][CG]GTAG[CG]GAGCTGCTCAAAG[CG]AGGT
GCTCAGCCCTGGGAGGCTCAGGG
SEQ ID NO: 5007
TT[CG]TTGATATTTGTGCTCACTTATTAG[CG]GTAAATTATTA
ATTTTAGATAAGCCCATCACAGAGTGAAGGCTCAATCCTTGAACA
GCCCAGGGAGAACTT[CG]TTGCTACTTTTGAAAAGCCCTTTGAT
GTGCCCAAAGTCTCCAGAAGGG[CG]ATGCCAAACCCACTCAACT
TTGACAAAGTGTGAAA[[CG]]TCTGTAAAAAGGAGTTCTCTGAT
GTGTGAGAGGAGA[[CG]]GAAC[CG]AGAAGAAAAGGC[[CG]]
TACTCTGCCCCC[CG][CG]GGACCCAGGTCCC[CG]CCTGCTGC
AGAG[CG]CACTCTG[[CG]]CA[[CG]]T[[CG]]AGC[CG][C
G]AAAGGTTCACAGAAGAAAACAAGAGAAAGAAGTAGCAGGCACT
GAGGAGC[CG]C[CG]G[CG]C[CG]G[CG]CCCAGGA[CG]GCA
[CG]TGACC[CG]GGGG[CG]GGGC[CG][CG][CG]GGC[CG][
CG]GGCCAGTGGATGGGGA[CG]GGGG[CG]GGGC[CG][CG]GC
T[CG]GGGGG[CG]GGGTCTGTGG[CG]G[CG]CAGCTTACAC[C
G][CG]GTCCCCTTATTTGGATCTG[CG]GGAATGTGGGCTGGAG
AGGTCCTGC[CG]TGGTACCAGCCTCCAGCCTGCCCCCAGGACTG
CCCCTGACCCAGG[CG][CG]CC[CG]CT
SEQ ID NO: 5008
TGTGCTCACTTATTAG[CG]GTAAATTATTAATTTTAGATAAGCC
CATCACAGAGTGAAGGCTCAATCCTTGAACAGCCCAGGGAGAACT
T[CG]TTGCTACTTTTGAAAAGCCCTTTGATGTGCCCAAAGTCTC
CAGAAGGG[CG]ATGCCAAACCCACTCAACTTTGACAAAGTGTGA
AA[[CG]]TCTGTAAAAAGGAGTTCTCTGATGTGTGAGAGGAGA[
[CG]]GAAC[CG]AGAAGAAAAGGC[[CG]]TACTCTGCCCCC[C
G][CG]GGACCCAGGTCCC[CG]CCTGCTGCAGAG[CG]CACTCT
G[[CG]]CA[[CG]]T[[CG]]AGC[[CG]][[CG]]AAAGGTTC
ACAGAAGAAAACAAGAGAAAGAAGTAGCAGGCACTGAGGAGC[CG
]C[CG]G[CG]C[CG]G[CG]CCCAGGA[CG]GCA[CG]TGACC[
CG]GGGG[CG]GGGC[CG][CG][CG]GGC[CG][CG]GGCCAGT
GGATGGGGA[CG]GGGG[CG]GGGC[CG][CG]GCT[CG]GGGGG
[CG]GGGTCTGTGG[CG]G[CG]CAGCTTACAC[CG][CG]GTCC
CCTTATTTGGATCTG[CG]GGAATGTGGGCTGGAGAGGTCCTGC[
CG]TGGTACCAGCCTCCAGCCTGCCCCCAGGACTGCCCCTGACCC
AGG[CG][CG]CC[CG]CTGCT[CG]GTGGCAG
SEQ ID NO: 5009
ATATTTGTGCTCACTTATTAG[CG]GTAAATTATTAATTTTAGAT
AAGCCCATCACAGAGTGAAGGCTCAATCCTTGAACAGCCCAGGGA
GAACTT[CG]TTGCTACTTTTGAAAAGCCCTTTGATGTGCCCAAA
GTCTCCAGAAGGG[CG]ATGCCAAACCCACTCAACTTTGACAAAG
TGTGAAA[[CG]]TCTGTAAAAAGGAGTTCTCTGATGTGTGAGAG
GAGA[[CG]]GAAC[CG]AGAAGAAAAGGC[[CG]]TACTCTGCC
CCC[CG][CG]GGACCCAGGTCCC[CG]CCTGCTGCAGAG[CG]C
ACTCTG[[CG]]CA[[CG]]T[[CG]]AGC[CG][[CG]]AAAGG
TTCACAGAAGAAAACAAGAGAAAGAAGTAGCAGGCACTGAGGAGC
[CG]C[CG]G[CG]C[CG]G[CG]CCCAGGA[CG]GCA[CG]TGA
CC[CG]GGGG[CG]GGGC[CG][CG][CG]GGC[CG][CG]GGCC
AGTGGATGGGGA[CG]GGGG[CG]GGGC[CG][CG]GCT[CG]GG
GGG[CG]GGGTCTGTGG[CG]G[CG]CAGCTTACAC[CG][CG]G
TCCCCTTATTTGGATCTG[[CG]]GGAATGTGGGCTGGAGAGGTC
CTGC[CG]TGGTACCAGCCTCCAGCCTGCCCCCAGGACTGCCCCT
GACCCAGG[CG][CG]CC[CG]CTGCT[CG]GT
SEQ ID NO: 5010
TGCTCACTTATTAG[CG]GTAAATTATTAATTTTAGATAAGCCCA
TCACAGAGTGAAGGCTCAATCCTTGAACAGCCCAGGGAGAACTT[
CG]TTGCTACTTTTGAAAAGCCCTTTGATGTGCCCAAAGTCTCCA
GAAGGG[CG]ATGCCAAACCCACTCAACTTTGACAAAGTGTGAAA
[[CG]]TCTGTAAAAAGGAGTTCTCTGATGTGTGAGAGGAGA[[C
G]]GAAC[CG]AGAAGAAAAGGC[[CG]]TACTCTGCCCCC[CG]
[CG]GGACCCAGGTCCC[CG]CCTGCTGCAGAG[CG]CACTCTG[
[CG]]CA[[CG]]T[[CG]]AGC[[CG]][[CG]]AAAGGTTCAC
AGAAGAAAACAAGAGAAAGAAGTAGCAGGCACTGAGGAGC[CG]C
[CG]G[CG]C[CG]G[CG]CCCAGGA[CG]GCA[CG]TGACC[CG
]GGGG[CG]GGGC[CG][CG][CG]GGC[CG][CG]GGCCAGTGG
ATGGGGA[CG]GGGG[CG]GGGC[CG][CG]GCT[CG]GGGGG[C
G]GGGTCTGTGG[CG]G[CG]CAGCTTACAC[CG][CG]GTCCCC
TTATTTGGATCTG[[CG]]GGAATGTGGGCTGGAGAGGTCCTGC[
CG]TGGTACCAGCCTCCAGCCTGCCCCCAGGACTGCCCCTGACCC
AGG[CG][CG]CC[CG]CTGCT[CG]GTGGCAGGA
SEQ ID NO: 5011
TTGATATTTGTGCTCACTTATTAG[CG]GTAAATTATTAATTTTA
GATAAGCCCATCACAGAGTGAAGGCTCAATCCTTGAACAGCCCAG
GGAGAACTT[CG]TTGCTACTTTTGAAAAGCCCTTTGATGTGCCC
AAAGTCTCCAGAAGGG[CG]ATGCCAAACCCACTCAACTTTGACA
AAGTGTGAAA[[CG]]TCTGTAAAAAGGAGTTCTCTGATGTGTGA
GAGGAGA[[CG]]GAAC[CG]AGAAGAAAAGGC[[CG]]TACTCT
GCCCCC[CG][CG]GGACCCAGGTCCC[CG]CCTGCTGCAGAG[C
G]CACTCTG[[CG]]CA[[CG]]T[[CG]]AGC[[CG]][[CG]]
AAAGGTTCACAGAAGAAAACAAGAGAAAGAAGTAGCAGGCACTGA
GGAGC[CG]C[CG]G[CG]C[CG]G[CG]CCCAGGA[CG]GCA[C
G]TGACC[CG]GGGG[CG]GGGC[CG][CG][CG]GGC[CG][CG
]GGCCAGTGGATGGGGA[CG]GGGG[CG]GGGC[CG][CG]GCT[
CG]GGGGG[CG]GGGTCTGTGG[CG]G[CG]CAGCTTACAC[CG]
[CG]GTCCCCTTATTTGGATCTG[[CG]]GGAATGTGGGCTGGAG
AGGTCCTGC[CG]TGGTACCAGCCTCCAGCCTGCCCCCAGGACTG
CCCCTGACCCAGG[CG][CG]CC[CG]CTGCTC
SEQ ID NO: 5012
TGTACACACACACAAAATATACCATACAGAGTGTAAGGTTCATGA
[CG]TCTTTCTTT[CG]TTGATATTTGTGCTCACTTATTAG[CG]
GTAAATTATTAATTTTAGATAAGCCCATCACAGAGTGAAGGCTCA
ATCCTTGAACAGCCCAGGGAGAACTT[CG]TTGCTACTTTTGAAA
AGCCCTTTGATGTGCCCAAAGTCTCCAGAAGGG[CG]ATGCCAAA
CCCACTCAACTTTGACAAAGTGTGAAA[[CG]]TCTGTAAAAAGG
AGTTCTCTGATGTGTGAGAGGAGA[[CG]]GAAC[CG]AGAAGAA
AAGGC[CG]TACTCTGCCCCC[CG][CG]GGACCCAGGTCCC[CG
]CCTGCTGCAGAG[CG]CACTCTG[[CG]]CA[[CG]]T[[CG]]
AGC[CG][CG]AAAGGTTCACAGAAGAAAACAAGAGAAAGAAGTA
GCAGGCACTGAGGAGC[CG]C[CG]G[CG]C[CG]G[CG]CCCAG
GA[CG]GCA[CG]TGACC[CG]GGGG[CG]GGGC[CG][CG][CG
]GGC[CG][CG]GGCCAGTGGATGGGGA[CG]GGGG[CG]GGGC[
CG][CG]GCT[CG]GGGGG[CG]GGGTCTGTGG[CG]G[CG]CAG
CTTACAC[CG][CG]GTCCCCTTATTTGGATCTG[[CG]]GGAAT
GTGGGCTGGAGAGGTCCTGC[CG]T
SEQ ID NO: 5013
ATACATGTATATGTAAAATATGTACACACACACAAAATATACCAT
ACAGAGTGTAAGGTTCATGA[CG]TCTTTCTTT[CG]TTGATATT
TGTGCTCACTTATTAG[CG]GTAAATTATTAATTTTAGATAAGCC
CATCACAGAGTGAAGGCTCAATCCTTGAACAGCCCAGGGAGAACT
T[CG]TTGCTACTTTTGAAAAGCCCTTTGATGTGCCCAAAGTCTC
CAGAAGGG[CG]ATGCCAAACCCACTCAACTTTGACAAAGTGTGA
AA[[CG]]TCTGTAAAAAGGAGTTCTCTGATGTGTGAGAGGAGA[
[CG]]GAAC[CG]AGAAGAAAAGGC[[CG]]TACTCTGCCCCC[C
G][CG]GGACCCAGGTCCC[CG]CCTGCTGCAGAG[CG]CACTCT
G[[CG]]CA[[CG]]T[[CG]]AGC[CG][CG]AAAGGTTCACAG
AAGAAAACAAGAGAAAGAAGTAGCAGGCACTGAGGAGC[CG]C[C
G]G[CG]C[CG]G[CG]CCCAGGA[CG]GCA[CG]TGACC[CG]G
GGG[CG]GGGC[CG][CG][CG]GGC[CG][CG]GGCCAGTGGAT
GGGGA[CG]GGGG[CG]GGGC[CG][CG]GCT[CG]GGGGG[CG]
GGGTCTGTGG[CG]G[CG]CAGCTTACAC[CG][CG]GTCCCCTT
ATTTGGATCTG[[CG]]GGAATGTG
SEQ ID NO: 5014
C[CG]GAGGAT[CG]CACT[CG]CTCATCCTTACACACCACACAC
ACACACACACACACACACACACACACACTGC[CG]CTAG[CG]CC
CCCCA[CG]CCACAAGC[CG]GAATG[CG]GAGGTCAGAGCCACT
GTCCACTTGAAAGCAGGAAAGCAGGGGCTGAAA[CG]CTGAGATG
GAGG[CG]GAGGCTCTGGCCTCATCTG[CG]AGGTT[CG]CACCT
TCC[CG]GAAGGCCCCCCCCTCCCC[CG][CG]TTTCCCCAGGG[
CG]AGCAAGGTCTAGCTTCTGAGACTC[CG]CTCTCTGGGCTAG[
CG]GTT[CG]AGCTGCAGGGGGG[[CG]]GGGA[CG]GATT[CG]
GAGGGAGCACTAGAGAGAGAGCC[CG][CG]CTCACTCCTGCCCT
CCT[CG]CAAAGAACTGGCCAGCCCCACT[CG][CG]TCCAGCCC
ACAACTAGGCTGC[CG]GGAC[CG]GGGAGCCAGACCCCAGGT[C
G]CTCCAACTC[CG]G[CG]AGGG[CG]GGGAGGGGAGTGACAGC
AGCAACTAG[CG][CG][CG]CTGGCAAAAGCACAGAGTCT[[CG
]]GAATAGGCTAAA[CG]GC[CG]G[CG][CG]GAGGGGTGGAGG
CAAGAGGGGG[CG]GAGGAGGAGTTGCC[CG]AG[CG]GAGAGGG
ATGA[CG]TCCTCCAGGGC
SEQ ID NO: 5015
CCCAGTCCCAGAACCTCCACCCCTTT[CG]AATTCTTCCCAA[CG
]GGCTGACCCTGCC[CG]G[CG]CCCAGGAG[CG]CCCTGGGTAT
CTCCTGGCTGCTCTCC[CG]AATCCTTG[CG][CG]C[CG][CG]
CCCCTACCAGGTTCACTGGGTGCA[CG]TAGC[CG]TTCTCATAG
[CG]GTCCTCCTGCAACAGCTGC[CG]CAGGTGAG[CG]ATGTAA
CTGGAAGCCAGC[CG]GAG[CG]TGTCCAGCTTGGAGAGCTTAGT
GT[CG]GGGGGCACCCAGGGCAGGCTGGTCTTGAGCCTGGAGAAG
GCTTTGCTCAGCA[CG][CG]CATC[CG]GGCA[CG]CTCA[CG]
GG[CG]TTGGC[CG][CG]TTC[CG]CTG[CG]ACTGCTTGCACT
CTG[CG]GCTGAGCCCTTGGC[CG]GGAGGGGCTTCTTGCCACCA
C[CG]CC[CG][CG]CTACCACCTG[CG]C[CG]C[CG]CCCCCA
GCCACA[CG]GGGC[CG]CTTCCTCTTGCAGCCTTC[CG][CG]C
TGC[CG]GCTGTGCCCAGAG[CG]CAG[CG]CTCCTCCT[CG]C[
CG]T[CG]GGGTCCTCCTCCTCTGC[CG]A[CG]AGTTGTCACTG
GG[CG]AGG[CG]TAGCTG[CG]CTCTA[CG]C[CG][CG]GAGG
GG[CG]GCCTCTTGGAGG[[CG]]GGGAC[CG]GGTACTCC
SEQ ID NO: 5016
GAATCCTTG[CG][CG]C[CG][CG]CCCCTACCAGGTTCACTGG
GTGCA[CG]TAGC[CG]TTCTCATAG[CG]GTCCTCCTGCAACAG
CTGC[CG]CAGGTGAG[CG]ATGTAACTGGAAGCCAGC[CG]GAG
[CG]TGTCCAGCTTGGAGAGCTTAGTGT[CG]GGGGGCACCCAGG
GCAGGCTGGTCTTGAGCCTGGAGAAGGCTTTGCTCAGCA[CG][C
G]CATC[[CG]]GGCA[CG]CTCA[CG]GG[CG]TTGGC[CG][C
G]TTC[CG]CTG[CG]ACTGCTTGCACTCTG[CG]GCTGAGCCCT
TGGC[CG]GGAGGGGCTTCTTGCCACCAC[CG]CC[CG][CG]CT
ACCACCTG[CG]C[CG]C[CG]CCCCCAGCCACA[CG]GGGC[CG
]CTTCCTCTTGCAGCCTTC[CG][CG]CTGC[CG]GCTGTGCCCA
GAG[CG]CAG[CG]CTCCTCCT[CG]C[CG]T[CG]GGGTCCTCC
TCCTCTGC[CG]A[CG]AGTTGTCACTGGG[CG]AGG[CG]TAGC
TG[CG]CTCTA[CG]C[CG][CG]GAGGGG[CG]GCCTCTTGGAG
G[[CG]]GGGAC[CG]GGTACTCC[CG]CTGCAGCCCC[CG]AAG
CTCCATCTCCTC[CG]GATCACTCAC[CG]AGCC[CG]TGGACAT
CC[CG]TTGTCCCCCTTGCCCACA[CG][CG]TCCTCTTTCCTCC
CC
SEQ ID NO: 5017
C[CG]AATCCTTG[CG][CG]C[CG][CG]CCCCTACCAGGTTCA
CTGGGTGCA[CG]TAGC[CG]TTCTCATAG[CG]GTCCTCCTGCA
ACAGCTGC[CG]CAGGTGAG[CG]ATGTAACTGGAAGCCAGC[CG
]GAG[CG]TGTCCAGCTTGGAGAGCTTAGTGT[CG]GGGGGCACC
CAGGGCAGGCTGGTCTTGAGCCTGGAGAAGGCTTTGCTCAGCA[C
G][CG]CATC[CG]GGCA[CG]CTCA[CG]GG[CG]TTGGC[CG]
[CG]TTC[CG]CTG[CG]ACTGCTTGCACTCTG[CG]GCTGAGCC
CTTGGC[CG]GGAGGGGCTTCTTGCCACCAC[CG]CC[CG][CG]
CTACCACCTG[CG]C[CG]C[CG]CCCCCAGCCACA[CG]GGGC[
CG]CTTCCTCTTGCAGCCTTC[CG][CG]CTGC[CG]GCTGTGCC
CAGAG[CG]CAG[CG]CTCCTCCT[CG]C[CG]T[CG]GGGTCCT
CCTCCTCTGC[CG]A[CG]AGTTGTCACTGGG[CG]AGG[CG]TA
GCTG[CG]CTCTA[CG]C[CG][CG]GAGGGG[CG]GCCTCTTGG
AGG[[CG]]GGGAC[CG]GGTACTCC[CG]CTGCAGCCCC[CG]A
AGCTCCATCTCCTC[CG]GATCACTCAC[CG]AGCC[CG]TGGAC
ATCC[CG]TTGTCCCCCTTGCCCACA[CG][CG]TCCTCTTTCCT
CC
SEQ ID NO: 5018
TCACT[CG][CG]GCC[CG]GG[CG][CG]G[CG]GTG[CG]AGC
TCCC[CG]CCTG[CG]GGA[CG]CA[CG]GAGAC[CG][CG]GTC
AG[CG][CG]C[CG]CCTGGC[CG]GCCCAG[CG][CG]CCCAGC
C[CG][CG]CCCAGCCC[CG]TCCACTCC[CG]TCCAGCCC[CG]
C[CG]CC[CG]GC[CG]CAGTCCCCAGGG[CG]CAAGCTGGG[CG
]GGTGC[CG]CACAGAGCCCCCTTGTCCT[CG]C[CG]CC[CG]C
CCC[CG]GCC[CG]GGT[CG]TTTC[CG]C[CG][CG]GTGGCCA
[CG]GCCC[CG]CCCT[CG]TTC[CG][CG]CC[CG]GACTGGGC
CA[CG]C[CG]GATAG[CG]GGAAACAAAAAAAGCC[[CG]]AGC
TGGAAACTTCAGAGAGGTTTAGTTT[CG]TTTCCCAGAAGCATCA
GTT[CG]GTCCCAAAA[CG]CTGCAAA[CG][CG][CG]CTGCCT
GCAGTAGGAGAGAGGAAAC[CG][CG]AAG[CG][CG]AGAAAAG
G[CG]CCCC[CG]TCCCCAAGCAGCC[CG][CG][CG]CCCTTCC
AGGGGCCAGACCTGCTCCATCCTGGA[CG]G[[CG]]AAA[CG]A
CCT[CG]GGAGACCC[[CG]]GTTAGGACCTTACTCCAGGGATCA
ACTGGAATCAAAATCCAAAGGATCCATGGCATCCAGTGCTCCAAT
TTTTAAGTCTTTTTTA[CG]CAGGAC
SEQ ID NO: 5019
CTCCC[CG]CCTG[CG]GGA[CG]CA[CG]GAGAC[CG][CG]GT
CAG[CG][CG]C[CG]CCTGGC[[CG]]GCCCAG[CG][CG]CCC
AGCC[[CG]][[CG]]CCCAGCCC[CG]TCCACTCC[CG]TCCAG
CCC[CG]C[CG]CC[CG]GC[CG]CAGTCCCCAGGG[CG]CAAGC
TGGG[CG]GGTGC[CG]CACAGAGCCCCCTTGTCCT[CG]C[CG]
CC[CG]CCCC[CG]GCC[CG]GGT[CG]TTTC[CG]C[CG][CG]
GTGGCCA[CG]GCCC[CG]CCCT[CG]TTC[CG][CG]CC[CG]G
ACTGGGCCA[CG]C[CG]GATAG[CG]GGAAACAAAAAAAGCC[[
CG]]AGCTGGAAACTTCAGAGAGGTTTAGTTT[CG]TTTCCCAGA
AGCATCAGTT[[CG]]GTCCCAAAA[[CG]]CTGCAAA[[CG]][
CG][CG]CTGCCTGCAGTAGGAGAGAGGAAAC[CG][CG]AAG[C
G][CG]AGAAAAGG[CG]CCCC[CG]TCCCCAAGCAGCC[CG][C
G][CG]CCCTTCCAGGGGCCAGACCTGCTCCATCCTGGA[CG]G[
[CG]]AAA[[CG]]ACCT[[CG]]GGAGACCC[[CG]]GTTAGGA
CCTTACTCCAGGGATCAACTGGAATCAAAATCCAAAGGATCCATG
GCATCCAGTGCTCCAATTTTTAAGTCTTTTTTA[CG]CAGGACCT
CTGCTCCTGGGGAGCCCTGGGAAGGG
SEQ ID NO: 5020
TGAGAGGAGA[[CG]]GAAC[CG]AGAAGAAAAGGC[[CG]]TAC
TCTGCCCCC[CG][CG]GGACCCAGGTCCC[CG]CCTGCTGCAGA
G[CG]CACTCTG[[CG]]CA[[CG]]T[[CG]]AGC[[CG]][[C
G]]AAAGGTTCACAGAAGAAAACAAGAGAAAGAAGTAGCAGGCAC
TGAGGAGC[CG]C[CG]G[CG]C[CG]G[CG]CCCAGGA[CG]GC
A[CG]TGACC[CG]GGGG[CG]GGGC[CG][CG][CG]GGC[CG]
[CG]GGCCAGTGGATGGGGA[CG]GGGG[CG]GGGC[CG][CG]G
CT[CG]GGGGG[CG]GGGTCTGTGG[CG]G[CG]CAGCTTACAC[
CG][CG]GTCCCCTTATTTGGATCTG[CG]GGAATGTGGGCTGGA
GAGGTCCTGC[CG]TGGTACCAGCCTCCAGCCTGCCCCCAGGACT
GCCCCTGACCCAGG[CG][CG]CC[CG]CTGCT[CG]GTGGCAGG
AGGGC[CG]G[CG]GAG[CG]CCATGGCCTGCATCCTGAAGGTAA
[CG]ACTTGGATCTGTGGCT[CG]GA[CG][CG]TGGTTGGCCAG
CCCCTTGCTGCTCAGAGGCA[CG]GAGTCAGC[[CG]][CG]GTC
CCAC[CG]CATCCTCATCTCAGTTGTCTTTTCCTGGCATTGATTT
TCTTTTTGTTCCTTTGGCAAATGCATATGGAGGGAGATTCTGAAA
CA
SEQ ID NO: 5021
GGCAACTTAAAACATGG[CG]CCC[CG]GG[CG]GGGGATTTGTG
CAAATTGG[[CG]]GAGAAGGGA[CG][CG]GGGAC[CG]AGT[C
G]CCCCTT[CG]GCCAGGGATCCCAGGGAGGCCCCCAGGC[CG]G
AGGC[CG]GGGCTCAGGCTCTG[CG][CG]C[CG]GCCCAGCCAC
TACTG[CG]C[CG][CG]G[CG]GG[CG]GAG[CG]GG[CG]GGG
GG[CG][CG]G[CG][CG]CAGGCT[CG]GCC[CG]GTGGGGGTC
C[CG]G[CG]AG[CG]GGAGGG[CG]GTTGGGGACCC[CG]GC[C
G][CG]C[CG]GG[CG][CG]GGGCT[CG]GGATT[CG]GGAGAC
[CG][CG][CG]G[CG]C[CG]AAGCCA[CG][[CG]]TCAGCCC
CACTGTCC[CG][CG][CG]CCT[CG]CCCCAGGCCT[CG]GGCT
CTTCCTC[CG]CACCT[CG]TAAAGC[CG]AGACCCCCT[CG]CA
GTCCCCCACTC[CG]AGAGG[CG]GAAAAGTTACCTGGGATCAGC
AGGGAGCC[CG]GG[CG][CG]C[CG][CG]G[CG]TGGGGACTA
GGCT[CG]GG[CG][CG][CG]TCCT[CG]G[CG]G[CG]GTG[C
G]CAGGAGACT[CG]GG[CG]TGGGGAGGAAGC[CG]CAGCCCAG
GGCTGCT[CG]C[CG]CTGTTCCCCC[CG]CCCCCTGTTGCAGGA
GACAC[CG]AGGCTC[CG][CG]GAGCTG[CG]G[[CG]]GGGGC
CCA[[CG]]
SEQ ID NO: 5022
AAGGCCCCCCCCTCCCC[CG][CG]TTTCCCCAGGG[CG]AGCAA
GGTCTAGCTTCTGAGACTC[CG]CTCTCTGGGCTAG[CG]GTT[C
G]AGCTGCAGGGGGG[CG]GGGA[CG]GATT[CG]GAGGGAGCAC
TAGAGAGAGAGCC[CG][CG]CTCACTCCTGCCCTCCT[CG]CAA
AGAACTGGCCAGCCCCACT[CG][CG]TCCAGCCCACAACTAGGC
TGC[CG]GGAC[CG]GGGAGCCAGACCCCAGGT[CG]CTCCAACT
C[CG]G[CG]AGGG[CG]GGGAGGGGAGTGACAGCAGCAACTAG[
CG][CG][CG]CTGGCAAAAGCACAGAGTCT[[CG]]GAATAGGC
TAAA[CG]GC[CG]G[CG][CG]GAGGGGTGGAGGCAAGAGGGGG
[CG]GAGGAGGAGTTGCC[CG]AG[CG]GAGAGGGATGA[CG]TC
CTCCAGGGCCTTGGGCTC[CG]AGGAAC[CG]AAGACTGGTG[CG
]CT[[CG]]C[CG]GGCCC[CG][CG]CACTAGCTCTGGGGTT[C
G]CAGAGG[CG]C[CG][CG]CC[CG]GCTTC[CG][[CG]]GCA
GCTCCAACACAG[CG][CG]CAGG[CG]GT[CG]ATCC[CG]GTC
AGCTTAAAC[CG]GG[CG]CAAGCCCTCTGTCCCCCTCAAAGCAC
CTCTCTGCT[CG]TCCCTAGTCTCCAGGTGCC[CG]TCTGA
SEQ ID NO: 5023
CAGTCTGCAACCTGGTGGCAGATGGAGATGGAACCAAGATTTACC
CTGGAAGTAGGATATTAGTCAATGGCTGTCACTGGGGTACAATGT
TCAGAA[CG]ATGCAAACAGACA[[CG]]GAGGGTTGATTCTTCT
TCAGGGAAATGGCTGGCCACTCCCTTGATTTGGGAGAAACTAAAC
TGGCCTTCT[CG]TTCCCAGTCCCAGAACCTCCACCCCTTT[CG]
AATTCTTCCCAA[CG]GGCTGACCCTGCC[CG]G[CG]CCCAGGA
G[CG]CCCTGGGTATCTCCTGGCTGCTCTCC[CG]AATCCTTG[C
G][CG]C[CG][[CG]]CCCCTACCAGGTTCACTGGGTGCA[CG]
TAGC[CG]TTCTCATAG[CG]GTCCTCCTGCAACAGCTGC[CG]C
AGGTGAG[CG]ATGTAACTGGAAGCCAGC[CG]GAG[CG]TGTCC
AGCTTGGAGAGCTTAGTGT[CG]GGGGGCACCCAGGGCAGGCTGG
TCTTGAGCCTGGAGAAGGCTTTGCTCAGCA[CG][CG]CATC[CG
]GGCA[CG]CTCA[CG]GG[CG]TTGGC[CG][CG]TTC[CG]CT
G[CG]ACTGCTTGCACTCTG[CG]GCTGAGCCCTTGGC[CG]GGA
GGGGCTTCTTGCCACCAC[CG]CC[CG][CG]CTACCACCTG[CG
]CC
SEQ ID NO: 5024
CCTCTG[CG]TCCAGGG[CG]TGTACTGAGCCCAGGCTAGCTGAG
[CG]AGGCTGCAGCCCCTG[[CG]]ATGGCC[CG]GGAAGAGGCA
AGTGC[CG][CG]GGGCCAGAG[CG]AGGAGGGACCAGCAGGG[C
G]GCCTGGACCCCCACTTGGC[CG][CG]CAGGCAGGAGGGG[CG
][CG]CCCTCTG[CG]GAG[CG][CG]G[CG]C[CG][CG]GCAG
C[CG]CAGCATCTGG[CG]CT[CG]CTCTCTCTGGC[CG]GAGAG
GC[CG][CG]ACACCTAG[CG][CG]GGGC[CG]AGGCTAGGAAG
GG[CG]GGC[CG]GGGAGGGAGGAGGGCACTGGGAGGAAGC[CG]
[CG]GG[[CG]]AGGAAGCCC[CG]CTCCTGC[CG]AGTGCCTGG
AGAGGAGATTCCCAGCCTTCCTGCCCAACC[CG]AAACTTTTCCA
ACACA[CG]CAGG[CG]TATTCCT[CG]CAGGATT[CG]G[CG]G
TCC[CG]AGT[CG]CAGTCAGGGAGGAGGCT[CG]GGAAGCACA[
[CG]]TGCCCTGGCACACTGGAGGAGGTACACAGGCACACCAGGA
GGGACAGGGCCTCTTGGGAGG[CG]GCTGAGGG[CG]TGAAGGCA
GCAGG[CG]GCCACACATC[CG]AAAGGGACTCCCTG[CG]AG[C
G]GGAT[CG]AG[CG]CTTAAAACCCTGCCACAGCTT
SEQ ID NO: 5025
TCTTTTCTTCTCT[CG]CACTCCACCTCCCACTGCCC[CG]CACC
CCCTTTAGCC[CG]GTGTTCCC[CG]CTC[CG]CTATGATTGA[C
G]TCTGGAAAGAAAGAGCTTTGTGAGGGGATGATTGTTATTAACT
TGTTATCCC[CG]G[CG]GGGGGG[CG]GGGAAC[CG]A[CG]TG
CC[CG]GGTGAG[CG]C[CG]GAGACC[CG]GAGCC[CG]GGGAG
[CG][CG]GGA[CG]AGCTACCAG[CG]CT[CG]GGTGG[CG]GC
[CG]CCAG[CG]GCCAG[CG]AAGGAGGCTG[CG][CG]CCAGCC
[CG]CC[CG][CG]G[CG]CC[CG]GGCTCAGG[CG]C[CG]TGA
[CG]GCTGCA[[CG]][CG]CTGCCC[CG]CACTCTGAGGGCCTT
CATTAGCT[CG]CTCCC[CG][CG]C[CG]AGGCTGGGG[CG]GG
CAGCA[CG]CT[CG]GAACTTCTGATCTGTTTCTCCATACTTTCT
CCCCCTCCTACTC[CG]CAGTGTCAGGGGCTCATGTCAGAGGAGT
G[CG]GG[CG]GACTACAGCCCTGG[CG]GC[CG]GGAGGACT[C
G]CAAAGG[CG]C[CG]GGGAAGAGGGACTGGTGAGTG[CG]GAG
GGGCT[CG][CG]TGCTGGCAAGACTGGC[[CG]]TGGGGAGG[C
G]G[CG]CAGGA[CG]AAGGGC[CG]CTGTCCC[CG][[CG]]CT
CCTTCCAGGGCC
SEQ ID NO: 5026
[CG]C[CG]TGA[CG]GCTGCA[CG][CG]CTGCCC[CG]CACTC
TGAGGGCCTTCATTAGCT[CG]CTCCC[CG][CG]C[CG]AGGCT
GGGG[CG]GGCAGCA[CG]CT[CG]GAACTTCTGATCTGTTTCTC
CATACTTTCTCCCCCTCCTACTC[CG]CAGTGTCAGGGGCTCATG
TCAGAGGAGTG[CG]GG[CG]GACTACAGCCCTGG[CG]GC[CG]
GGAGGACT[CG]CAAAGG[CG]C[CG]GGGAAGAGGGACTGGTGA
GTG[CG]GAGGGGCT[CG][CG]TGCTGGCAAGACTGGC[[CG]]
TGGGGAGG[CG]G[CG]CAGGA[CG]AAGGGC[CG]CTGTCCC[C
G][[CG]]CTCCTTCCAGGGCCCAGCTGGCCCCTCCTGCAGCAGA
CAGAGGA[CG]GGG[CG]GAGAGGGTCTGGGTGGGGTGGGAAGCT
TAGCTGGCC[CG][CG]AGGAGGGCAGGGC[CG]CAAGACTTCTG
GGGTC[CG]AATAGGTCAGGGGT[CG]GCAGCCCCAG[CG]GGTG
[CG]TAGCCAGAGCTTGGACT[CG]GGGAAGAGGG[CG]AGAAT[
CG]TGACCTCAAGAAGCTGGGGGTGGGGTGGGGTGGCCAGGGGGA
AGGGAGGGGAGG[CG]GTGGGGG[CG]CCCTTGTGGGTAGGTGGG
ATG[CG]AAGGGGAGGAGGGG
SEQ ID NO: 5027
CAGGGCAGGCTGGTCTTGAGCCTGGAGAAGGCTTTGCTCAGCA[C
G][CG]CATC[CG]GGCA[CG]CTCA[CG]GG[CG]TTGGC[CG]
[CG]TTC[CG]CTG[CG]ACTGCTTGCACTCTG[CG]GCTGAGCC
CTTGGC[CG]GGAGGGGCTTCTTGCCACCAC[CG]CC[CG][CG]
CTACCACCTG[CG]C[CG]C[CG]CCCCCAGCCACA[CG]GGGC[
CG]CTTCCTCTTGCAGCCTTC[CG][CG]CTGC[CG]GCTGTGCC
CAGAG[CG]CAG[CG]CTCCTCCT[CG]C[CG]T[CG]GGGTCCT
CCTCCTCTGC[CG]A[CG]AGTTGTCACTGGG[CG]AGG[CG]TA
GCTG[CG]CTCTA[CG]C[CG][CG]GAGGGG[CG]GCCTCTTGG
AGG[CG]GGGAC[CG]GGTACTCC[CG]CTGCAGCCCC[CG]AAG
CTCCATCTCCTC[CG]GATCACTCAC[CG]AGCC[CG]TGGACAT
CC[CG]TTGTCCCCCTTGCCCACA[CG][CG]TCCTCTTTCCTCC
CCCCTGGCCAGTCT[CG]CTGTCTC[CG]CCTTC[CG]CTCCCTG
G[CG]GAGG[CG]GAGGCCAGAGAG[CG]CTCCAAGGAAGACTAA
AAACCCAGGC[CG]GGAAG[CG][CG]GGGTGAGAAAG[CG]AGG
TGGGTGG[CG]AGAG[CG]TGAG[CG]CCCCTCTGCTGACCC[CG
]G
SEQ ID NO: 5028
AGGCTG[CG][CG]CCAGCC[CG]CC[CG][CG]G[CG]CC[CG]
GGCTCAGG[CG]C[CG]TGA[CG]GCTGCA[CG][CG]CTGCCC[
CG]CACTCTGAGGGCCTTCATTAGCT[CG]CTCCC[CG][CG][C
G]AGGCTGGGG[CG]GGCAGCA[CG]CT[CG]GAACTTCTGATCT
GTTTCTCCATACTTTCTCCCCCTCCTACTC[CG]CAGTGTCAGGG
GCTCATGTCAGAGGAGTG[CG]GG[CG]GACTACAGCCCTGG[CG
]GC[CG]GGAGGACT[CG]CAAAGG[CG]C[CG]GGGAAGAGGGA
CTGGTGAGTG[CG]GAGGGGCT[CG][CG]TGCTGGCAAGACTGG
C[[CG]]TGGGGAGG[CG]G[CG]CAGGA[CG]AAGGGC[CG]CT
GTCCC[CG][[CG]]CTCCTTCCAGGGCCCAGCTGGCCCCTCCTG
CAGCAGACAGAGGA[CG]GGG[CG]GAGAGGGTCTGGGTGGGGTG
GGAAGCTTAGCTGGCC[CG][CG]AGGAGGGCAGGGC[CG]CAAG
ACTTCTGGGGTC[CG]AATAGGTCAGGGGT[CG]GCAGCCCCAG[
CG]GGTG[CG]TAGCCAGAGCTTGGACT[CG]GGGAAGAGGG[CG
]AGAAT[CG]TGACCTCAAGAAGCTGGGGGGGGGTGGGGTGGCCA
GGGGGAAGGGAGGGGAGG[CG]GTGGGGG
SEQ ID NO: 5029
TGCTCAGCA[CG][CG]CATC[CG]GGCA[CG]CTCA[CG]GG[C
G]TTGGC[CG][CG]TTC[CG]CTG[CG]ACTGCTTGCACTCTG[
CG]GCTGAGCCCTTGGC[CG]GGAGGGGCTTCTTGCCACCAC[CG
]CC[CG][CG]CTACCACCTG[CG]C[CG]C[CG]CCCCCAGCCA
CA[CG]GGGC[CG]CTTCCTCTTGCAGCCTTC[CG][CG]CTGC[
CG]GCTGTGCCCAGAG[CG]CAG[CG]CTCCTCCT[CG]C[CG]T
[CG]GGGTCCTCCTCCTCTGC[CG]A[CG]AGTTGTCACTGGG[C
G]AGG[CG]TAGCTG[CG]CTCTA[CG]C[CG][CG]GAGGGG[C
G]GCCTCTTGGAGG[[CG]]GGGAC[CG]GGTACTCC[CG]CTGC
AGCCCC[CG]AAGCTCCATCTCCTC[CG]GATCACTCAC[CG]AG
CC[CG]TGGACATCC[CG]TTGTCCCCCTTGCCCACA[CG][CG]
TCCTCTTTCCTCCCCCCTGGCCAGTCT[CG]CTGTCTC[CG]CCT
TC[CG]CTCCCTGG[CG]GAGG[CG]GAGGCCAGAGAG[CG]CTC
CAAGGAAGACTAAAAACCCAGGC[CG]GGAAG[CG][CG]GGGTG
AGAAAG[CG]AGGTGGGTGG[CG]AGAG[CG]TGAG[CG]CCCCT
CTGCTGACCC[CG]GGGAG[CG]TGGACTA[CG]AGTTGG[CG]C
CCAAGTCCAG
SEQ ID NO: 5030
AGCAG[CG]G[CG]GAGC[CG]GAGC[CG]CCAGAGCCAGAGC[C
G]GAGCTG[CG]G[CG]G[CG]TGGACC[CG]GCAGGGG[CG]GG
CCT[CG]GACAGCAGAGA[CG]CAG[CG]GGCC[CG]GC[CG]AC
[CG]GGCTG[CG]GGAGTGGCCC[CG]GGCATGGGG[CG]GC[CG
]G[CG]GGGGC[CG][CG]GG[CG]GG[CG]CTG[CG]CTT[CG]
GGCTGG[CG]G[CG]GGCTG[CG]TGAG[CG]GC[CG]GGA[CG]
CAGGATG[CG]CTC[CG]AGGGTG[CG]GCCCC[CG]GGC[CG]G
[CG]G[CG]C[CG]CTGTG[CG]GGG[CG]CTGAGCCTGCTGCTG
GG[CG][CG]CTGCTGGGCAAAGGTAAGGC[[CG]]GGG[CG]GG
TGCCTGG[CG][CG]C[CG]A[CG]CATG[CG]AACTTCC[CG]G
TTC[CG]GGGGAGCT[CG]GGGTGAGGGCAGC[CG]AAG[CG]AG
GGGTG[CG]G[CG]GGGGCCT[CG]G[CG]GGGGCT[CG]GATCC
CATCAACAG[CG]CCTCTG[CG]GGTGC[CG]AGC[CG]CCCCTA
ATCCAGAAGGG[CG]CTCCCAGCTGGG[CG]C[CG]GGATC[CG]
GGCAAGTTAGGCTGGTCC[CG]AGTCC[CG]AATCTCCCACCC[C
G]GACCCCCCAGACCCAGAGCTGCCC[CG][CG]GATGAGGAGGG
GG[CG]TGGCTGAGGGACA[CG]GTGTTTGTTCCCC
SEQ ID NO: 5031
T[CG][CG]GGCACTCACTCT[CG]CTCACACTACAATCAGATCC
TCCATG[CG]GCTTTCATATACTCTGTCACACT[CG]CAGCCAGA
ATCCCCT[CG]GATGGACTCTTGCAAACCTCCAGCCCCATCC[CG
]GGATC[CG]TG[CG]TCTTCTACAGCCACCACTTGGGCCAG[CG
]TCCAGGA[CG]CCCAAG[CG]TAACCTCCCCACTCTAAAC[CG]
TTAGGAATCAGTGGCTGGACTCAC[CG][[CG]]TCATAAAAGCA
TAAACTGTGACC[CG]ATGAGTC[CG][CG]CACT[CG]GAAAAC
AAGAGAA[CG]CTCCCA[CG]TCTC[CG]GG[[CG]]CAGCTTGT
GCCAG[CG]ACTACTAGGTCAGGGGG[CG]CAGCTGAGAGAATTC
AAGCAACCTCCC[CG]CAAC[CG]GTTC[CG]C[CG][[CG]]TT
TGTGGGCTGGTAGCC[CG]GAATACATTTCCCAGAGGCCTT[[CG
]][[CG]]GC[CG]A[CG]TGCTT[CG][CG]CAGGAA[CG]CAG
C[CG]CCTCC[CG]ACTGGAGGA[CG][CG]GTAG[CG]GAGCTG
CTCAAAG[CG]AGGTGCTCAGCCCTGGGAGGCTCAGGGCTGCC[[
CG]]AGGGGCTTAGTCCCAGAGCCAGTGCTGTCAGGGGCTGAGCA
TGTGAATAGGGCAGT[[CG]]GAAGACCATG
SEQ ID NO: 5032
[CG]CCCTGGAATTGC[CG][CG]G[CG]G[CG]GATGCAGAGGC
CAACCCAGAGTGCAACCATGGAGA[CG][CG]A[CG]TGTGTCCC
ATAGTAACCTGTTACAACATTATTTATAAGCTGCCATCCCTAGCC
TTCCCC[CG]CTTCCCCTC[CG]CTCCCT[CG]CCAGACTTG[CG
]GG[CG]A[CG]GGCACAGC[CG][CG]TCTGGCTCTTCC[CG]G
CCTCTCCCTTCTCT[CG][CG]GG[CG]CAGC[CG]ATCAATAGT
TAACACC[CG]GCTG[CG]GA[CG]G[CG]GCTCCATC[CG][CG
]GCAATCAC[CG]TAGTGCTTGTTTGTGGAAGC[CG]AG[[CG]]
TG[CG]TG[CG]C[CG][CG][CG][CG]CACCCAGTCCAG[CG]
[CG]GAGTGGG[CG]TCTACC[CG]AGGAGGGGTGTCTGGGGAGG
GGCTGCCCT[CG]TTACCCAAACAGTTTG[CG]CT[CG]CTTAAC
CTTGATGCAGCT[CG]AGGCTTCCCAGTCCAGCTCAGTTCAGACA
GAAAACCTGG[CG][CG][CG][CG][CG][CG]CACACACACA[
CG]CCTCCCCTGG[CG]T[CG]C[CG]CC[CG]GC[CG]GGTCCC
TGCCCTTAGGGACCAGAG[CG]G[CG]AC[CG]CTGCACCC[CG]
CAC[CG]CCTGCTGGAGGAGCCCC[CG]GAGC[CG]GGGC[CG]A
GC[CG]C[CG]G
SEQ ID NO: 5033
TCTG[CG]CCCTGGAATTGC[CG][CG]G[CG]G[CG]GATGCAG
AGGCCAACCCAGAGTGCAACCATGGAGA[CG][CG]A[CG]TGTG
TCCCATAGTAACCTGTTACAACATTATTTATAAGCTGCCATCCCT
AGCCTTCCCC[CG]CTTCCCCTC[CG]CTCCCT[CG]CCAGACTT
G[CG]GG[CG]A[CG]GGCACAGC[CG][CG]TCTGGCTCTTCC[
CG]GCCTCTCCCTTCTCT[CG][CG]GG[CG]CAGC[CG]ATCAA
TAGTTAACACC[CG]GCTG[CG]GA[CG]G[CG]GCTCCATC[CG
][CG]GCAATCAC[CG]TAGTGCTTGTTTGTGGAAGC[CG]AG[C
G]TG[CG]TG[CG]C[CG][CG][CG][CG]CACCCAGTCCAG[C
G][CG]GAGTGGG[CG]TCTACC[CG]AGGAGGGGTGTCTGGGGA
GGGGCTGCCCT[[CG]]TTACCCAAACAGTTTG[CG]CT[CG]CT
TAACCTTGATGCAGCT[CG]AGGCTTCCCAGTCCAGCTCAGTTCA
GACAGAAAACCTGG[CG][CG][CG][CG][CG][CG]CACACAC
ACA[CG]CCTCCCCTGG[CG]T[CG]C[CG]CC[CG]GC[CG]GG
TCCCTGCCCTTAGGGACCAGAG[CG]G[CG]AC[CG]CTGCACCC
[CG]CAC[CG]CCTGCTGGAGGAGCCCC[CG]GAGC[CG]GGGC[
CG]AGC[CG]CC
SEQ ID NO: 5034
TGCACCTGGGCTATGAGGCTGGAAGCAGAAAGGGGG[CG]CCAGA
AAG[CG]CA[CG]C[CG][CG]AGGGC[CG]G[CG]GGAAATGTA
GTCTCA[CG]C[CG]GTAAAGCCACAGAG[CG]A[CG]ATGAGG[
CG]AGA[CG]TCTG[CG]TTCTTCTCAGGCCTGAGACCCAAC[CG
]GCC[[CG]]GGCACCAGCTCCTGCTGGACCCCAGAG[CG]CCTG
GAAAGCTC[CG]GAACCTCTCACCTGC[CG]CCTCCT[CG]GGTC
CAAGTGCCAC[CG][CG]GC[CG][CG]CCCC[CG]GCAGCCCAG
GG[CG][CG]CTTCCACCA[CG]GTAC[CG]GTGGATT[CG]C[C
G]TG[[CG]]CAGC[CG]GAAGATGG[CG]CAGA[CG]CACAAAG
CACAC[CG]ATGCTG[CG]CCATGATAGGGC[CG]G[CG]C[[CG
]]CAGTGTCTTC[CG]GGAAACATAGTCTTTAGG[[CG]]TAAAG
GCAGCAGCC[CG]GCCTTGAAGC[CG]GATCT[CG][CG]ATGTT
TCAGGGTGAGC[CG]GA[CG]CAGG[CG]TGCCTG[CG]CAGTG[
CG][CG]GAGGAGTGCTGTT[[CG]]TGTGTT[CG]AGTCCCTGG
GTTGCTTCCTGGGGTCTGTGGTGCTGGGTGTGCTATCTG[[CG]]
TGTGATTCTCTAG[CG]AGAGATTGTGGG[CG]AGTGAC[CG]AG
TGGGCAAG
SEQ ID NO: 5035
G[CG]GGAAATGTAGTCTCA[CG]C[CG]GTAAAGCCACAGAG[C
G]A[CG]ATGAGG[CG]AGA[CG]TCTG[CG]TTCTTCTCAGGCC
TGAGACCCAAC[CG]GCC[CG]GGCACCAGCTCCTGCTGGACCCC
AGAG[CG]CCTGGAAAGCTC[CG]GAACCTCTCACCTGC[CG]CC
TCCT[CG]GGTCCAAGTGCCAC[CG][CG]GC[CG][CG]CCCC[
CG]GCAGCCCAGGG[CG][CG]CTTCCACCA[CG]GTAC[CG]GT
GGATT[CG]C[CG]TG[[CG]]CAGC[CG]GAAGATGG[CG]CAG
A[CG]CACAAAGCACAC[CG]ATGCTG[[CG]]CCATGATAGGGC
[CG]G[CG]C[CG]CAGTGTCTTC[CG]GGAAACATAGTCTTTAG
G[[CG]]TAAAGGCAGCAGCC[[CG]]GCCTTGAAGC[CG]GATC
T[CG][CG]ATGTTTCAGGGTGAGC[CG]GA[CG]CAGG[CG]TG
CCTG[CG]CAGTG[CG][CG]GAGGAGTGCTGTT[[CG]]TGTGT
T[CG]AGTCCCTGGGTTGCTTCCTGGGGTCTGTGGTGCTGGGTGT
GCTATCTG[CG]TGTGATTCTCTAG[CG]AGAGATTGTGGG[CG]
AGTGAC[CG]AGTGGGCAAGGGGC[CG]TCACTGTGTGTG[CG]T
GATTTTGACAGTGTGTGGTGGTAGCTTCTGACTC[CG][CG]TGG
GT
SEQ ID NO: 5036
GATC[CG]TG[CG]TCTTCTACAGCCACCACTTGGGCCAG[CG]T
CCAGGA[CG]CCCAAG[CG]TAACCTCCCCACTCTAAAC[CG]TT
AGGAATCAGTGGCTGGACTCAC[CG][[CG]]TCATAAAAGCATA
AACTGTGACC[CG]ATGAGTC[CG][CG]CACT[CG]GAAAACAA
GAGAA[CG]CTCCCA[CG]TCTC[CG]GG[[CG]]CAGCTTGTGC
CAG[CG]ACTACTAGGTCAGGGGG[CG]CAGCTGAGAGAATTCAA
GCAACCTCCC[CG]CAAC[CG]GTTC[CG]C[CG][CG]TTTGTG
GGCTGGTAGCC[CG]GAATACATTTCCCAGAGGCCTT[[CG]][[
CG]]GC[[CG]]A[CG]TGCTT[CG][CG]CAGGAA[CG]CAGC[
CG]CCTCC[CG]ACTGGAGGA[CG][CG]GTAG[CG]GAGCTGCT
CAAAG[CG]AGGTGCTCAGCCCTGGGAGGCTCAGGGCTGCC[[CG
]]AGGGGCTTAGTCCCAGAGCCAGTGCTGTCAGGGGCTGAGCATG
TGAATAGGGCAGT[[CG]]GAAGACCATGTGTGTCTGTGGCTTTT
AAATACACCC[CG]AA[[CG]]TCTATCTCTTTTAACTGGAGTAT
TTAATTTATTTACATTTAATATAGCCACTGATAGATTTAAAGCTG
CCATTTTATTATATTTTCTTTTT
SEQ ID NO: 5037
TC[CG]TG[CG]TCTTCTACAGCCACCACTTGGGCCAG[CG]TCC
AGGA[CG]CCCAAG[CG]TAACCTCCCCACTCTAAAC[CG]TTAG
GAATCAGTGGCTGGACTCAC[CG][CG]TCATAAAAGCATAAACT
GTGACC[CG]ATGAGTC[CG][CG]CACT[CG]GAAAACAAGAGA
A[CG]CTCCCA[CG]TCTC[CG]GG[[CG]]CAGCTTGTGCCAG[
CG]ACTACTAGGTCAGGGGG[CG]CAGCTGAGAGAATTCAAGCAA
CCTCCC[CG]CAAC[CG]GTTC[CG]C[CG][CG]TTTGTGGGCT
GGTAGCC[CG]GAATACATTTCCCAGAGGCCTT[[CG]][CG]GC
[[CG]]A[[CG]]TGCTT[CG][CG]CAGGAA[CG]CAGC[CG]C
CTCC[CG]ACTGGAGGA[CG][CG]GTAG[CG]GAGCTGCTCAAA
G[CG]AGGTGCTCAGCCCTGGGAGGCTCAGGGCTGCC[[CG]]AG
GGGCTTAGTCCCAGAGCCAGTGCTGTCAGGGGCTGAGCATGTGAA
TAGGGCAGT[CG]GAAGACCATGTGTGTCTGTGGCTTTTAAATAC
ACCC[CG]AA[CG]TCTATCTCTTTTAACTGGAGTATTTAATTTA
TTTACATTTAATATAGCCACTGATAGATTTAAAGCTGCCATTTTA
TTATATTTTCTTTTTTT
SEQ ID NO: 5038
GTCTTCTACAGCCACCACTTGGGCCAG[CG]TCCAGGA[CG]CCC
AAG[CG]TAACCTCCCCACTCTAAAC[CG]TTAGGAATCAGTGGC
TGGACTCAC[CG][CG]TCATAAAAGCATAAACTGTGACC[CG]A
TGAGTC[CG][CG]CACT[CG]GAAAACAAGAGAA[CG]CTCCCA
[CG]TCTC[CG]GG[[CG]]CAGCTTGTGCCAG[CG]ACTACTAG
GTCAGGGGG[CG]CAGCTGAGAGAATTCAAGCAACCTCCC[CG]C
AAC[CG]GTTC[CG]C[CG][[CG]]TTTGTGGGCTGGTAGCC[C
G]GAATACATTTCCCAGAGGCCTT[[CG]][[CG]]GC[[CG]]A
[[CG]]TGCTT[CG][CG]CAGGAA[CG]CAGC[CG]CCTCC[CG
]ACTGGAGGA[CG][CG]GTAG[CG]GAGCTGCTCAAAG[CG]AG
GTGCTCAGCCCTGGGAGGCTCAGGGCTGCC[[CG]]AGGGGCTTA
GTCCCAGAGCCAGTGCTGTCAGGGGCTGAGCATGTGAATAGGGCA
GT[[CG]]GAAGACCATGTGTGTCTGTGGCTTTTAAATACACCC[
CG]AA[[CG]]TCTATCTCTTTTAACTGGAGTATTTAATTTATTT
ACATTTAATATAGCCACTGATAGATTTAAAGCTGCCATTTTATTA
TATTTTCTTTTTTTTTTTTAG
SEQ ID NO: 5039
AG[CG]CA[CG]C[CG][CG]AGGGC[CG]G[CG]GGAAATGTAG
TCTCA[CG]C[CG]GTAAAGCCACAGAG[CG]A[CG]ATGAGG[C
G]AGA[CG]TCTG[CG]TTCTTCTCAGGCCTGAGACCCAAC[CG]
GCC[[CG]]GGCACCAGCTCCTGCTGGACCCCAGAG[CG]CCTGG
AAAGCTC[CG]GAACCTCTCACCTGC[CG]CCTCCT[CG]GGTCC
AAGTGCCAC[CG][CG]GC[CG][CG]CCCC[CG]GCAGCCCAGG
G[CG][CG]CTTCCACCA[CG]GTAC[CG]GTGGATT[CG]C[CG
]TG[[CG]]CAGC[CG]GAAGATGG[CG]CAGA[CG]CACAAAGC
ACAC[CG]ATGCTG[CG]CCATGATAGGGC[CG]G[CG]C[[CG]
]CAGTGTCTTC[[CG]]GGAAACATAGTCTTTAGG[[CG]]TAAA
GGCAGCAGCC[[CG]]GCCTTGAAGC[CG]GATCT[CG][CG]AT
GTTTCAGGGTGAGC[CG]GA[CG]CAGG[CG]TGCCTG[CG]CAG
TG[CG][CG]GAGGAGTGCTGTT[CG]TGTGTT[CG]AGTCCCTG
GGTTGCTTCCTGGGGTCTGTGGTGCTGGGTGTGCTATCTG[[CG]
]TGTGATTCTCTAG[CG]AGAGATTGTGGG[CG]AGTGAC[CG]A
GTGGGCAAGGGGC[CG]TCACTGTGTGTG[CG]TGATTTTGACAG
TGTGTGGTGGTA
SEQ ID NO: 5040
GTCTCA[CG]C[CG]GTAAAGCCACAGAG[CG]A[CG]ATGAGG[
CG]AGA[CG]TCTG[CG]TTCTTCTCAGGCCTGAGACCCAAC[CG
]GCC[[CG]]GGCACCAGCTCCTGCTGGACCCCAGAG[CG]CCTG
GAAAGCTC[CG]GAACCTCTCACCTGC[CG]CCTCCT[CG]GGTC
CAAGTGCCAC[CG][CG]GC[CG][CG]CCCC[CG]GCAGCCCAG
GG[CG][CG]CTTCCACCA[CG]GTAC[CG]GTGGATT[CG]C[C
G]TG[[CG]]CAGC[CG]GAAGATGG[CG]CAGA[CG]CACAAAG
CACAC[CG]ATGCTG[CG]CCATGATAGGGC[CG]G[CG]C[CG]
CAGTGTCTTC[CG]GGAAACATAGTCTTTAGG[[CG]]TAAAGGC
AGCAGCC[CG]GCCTTGAAGC[CG]GATCT[CG][CG]ATGTTTC
AGGGTGAGC[CG]GA[CG]CAGG[CG]TGCCTG[CG]CAGTG[CG
][CG]GAGGAGTGCTGTT[[CG]]TGTGTT[CG]AGTCCCTGGGT
TGCTTCCTGGGGTCTGTGGTGCTGGGTGTGCTATCTG[[CG]]TG
TGATTCTCTAG[CG]AGAGATTGTGGG[CG]AGTGAC[CG]AGTG
GGCAAGGGGC[CG]TCACTGTGTGTG[CG]TGATTTTGACAGTGT
GTGGTGGTAGCTTCTGACTC[CG][CG]TGGGT[CG]TTGAATGT
AT
SEQ ID NO: 5041
TG[CG]TCTTCTACAGCCACCACTTGGGCCAG[CG]TCCAGGA[C
G]CCCAAG[CG]TAACCTCCCCACTCTAAAC[CG]TTAGGAATCA
GTGGCTGGACTCAC[CG][CG]TCATAAAAGCATAAACTGTGACC
[CG]ATGAGTC[CG][CG]CACT[CG]GAAAACAAGAGAA[CG]C
TCCCA[CG]TCTC[CG]GG[CG]CAGCTTGTGCCAG[CG]ACTAC
TAGGTCAGGGGG[CG]CAGCTGAGAGAATTCAAGCAACCTCCC[C
G]CAAC[CG]GTTC[CG]C[CG][[CG]]TTTGTGGGCTGGTAGC
C[CG]GAATACATTTCCCAGAGGCCTT[CG][[CG]]GC[CG]A[
CG]TGCTT[CG][CG]CAGGAA[CG]CAGC[CG]CCTCC[CG]AC
TGGAGGA[CG][CG]GTAG[CG]GAGCTGCTCAAAG[CG]AGGTG
CTCAGCCCTGGGAGGCTCAGGGCTGCC[[CG]]AGGGGCTTAGTC
CCAGAGCCAGTGCTGTCAGGGGCTGAGCATGTGAATAGGGCAGT[
[CG]]GAAGACCATGTGTGTCTGTGGCTTTTAAATACACCC[CG]
AA[CG]TCTATCTCTTTTAACTGGAGTATTTAATTTATTTACATT
TAATATAGCCACTGATAGATTTAAAGCTGCCATTTTATTATATTT
TCTTTTTTTTTTT
SEQ ID NO: 5042
[CG]AGA[CG]TCTG[CG]TTCTTCTCAGGCCTGAGACCCAAC[C
G]GCC[CG]GGCACCAGCTCCTGCTGGACCCCAGAG[CG]CCTGG
AAAGCTC[CG]GAACCTCTCACCTGC[CG]CCTCCT[CG]GGTCC
AAGTGCCAC[CG][CG]GC[CG][CG]CCCC[CG]GCAGCCCAGG
G[CG][CG]CTTCCACCA[CG]GTAC[CG]GTGGATT[CG]C[CG
]TG[[CG]]CAGC[CG]GAAGATGG[CG]CAGA[CG]CACAAAGC
ACAC[CG]ATGCTG[[CG]]CCATGATAGGGC[CG]G[CG]C[CG
]CAGTGTCTTC[CG]GGAAACATAGTCTTTAGG[[CG]]TAAAGG
CAGCAGCC[CG]GCCTTGAAGC[CG]GATCT[[CG]][CG]ATGT
TTCAGGGTGAGC[CG]GA[CG]CAGG[CG]TGCCTG[CG]CAGTG
[CG][CG]GAGGAGTGCTGTT[[CG]]TGTGTT[CG]AGTCCCTG
GGTTGCTTCCTGGGGTCTGTGGTGCTGGGTGTGCTATCTG[CG]T
GTGATTCTCTAG[CG]AGAGATTGTGGG[CG]AGTGAC[CG]AGT
GGGCAAGGGGC[CG]TCACTGTGTGTG[CG]TGATTTTGACAGTG
TGTGGTGGTAGCTTCTGACTC[CG][CG]TGGGT[CG]TTGAATG
TATGACTGGGAC[CG]TTTAG[CG]GTGGATACACAACTGTGT
SEQ ID NO: 5043
CAGAG[CG]A[CG]ATGAGG[CG]AGA[CG]TCTG[CG]TTCTTC
TCAGGCCTGAGACCCAAC[CG]GCC[[CG]]GGCACCAGCTCCTG
CTGGACCCCAGAG[CG]CCTGGAAAGCTC[CG]GAACCTCTCACC
TGC[CG]CCTCCT[CG]GGTCCAAGTGCCAC[CG][CG]GC[CG]
[CG]CCCC[CG]GCAGCCCAGGG[CG][CG]CTTCCACCA[CG]G
TAC[CG]GTGGATT[CG]C[CG]TG[[CG]]CAGC[CG]GAAGAT
GG[CG]CAGA[CG]CACAAAGCACAC[CG]ATGCTG[CG]CCATG
ATAGGGC[CG]G[CG]C[[CG]]CAGTGTCTTC[CG]GGAAACAT
AGTCTTTAGG[CG]TAAAGGCAGCAGCC[[CG]]GCCTTGAAGC[
CG]GATCT[CG][CG]ATGTTTCAGGGTGAGC[CG]GA[CG]CAG
G[CG]TGCCTG[CG]CAGTG[CG][CG]GAGGAGTGCTGTT[[CG
]]TGTGTT[CG]AGTCCCTGGGTTGCTTCCTGGGGTCTGTGGTGC
TGGGTGTGCTATCTG[[CG]]TGTGATTCTCTAG[CG]AGAGATT
GTGGG[CG]AGTGAC[CG]AGTGGGCAAGGGGC[CG]TCACTGTG
TGTG[CG]TGATTTTGACAGTGTGTGGTGGTAGCTTCTGACTC[C
G][CG]TGGGT[CG]TTGAATGTATGACTGGGAC[CG]TTTAG[C
G]GT
SEQ ID NO: 5044
TCAGGCCTGAGACCCAAC[CG]GCC[CG]GGCACCAGCTCCTGCT
GGACCCCAGAG[CG]CCTGGAAAGCTC[CG]GAACCTCTCACCTG
C[CG]CCTCCT[CG]GGTCCAAGTGCCAC[CG][CG]GC[CG][C
G]CCCC[CG]GCAGCCCAGGG[CG][CG]CTTCCACCA[CG]GTA
C[CG]GTGGATT[CG]C[CG]TG[CG]CAGC[CG]GAAGATGG[C
G]CAGA[CG]CACAAAGCACAC[CG]ATGCTG[CG]CCATGATAG
GGC[CG]G[CG]C[CG]CAGTGTCTTC[[CG]]GGAAACATAGTC
TTTAGG[CG]TAAAGGCAGCAGCC[[CG]]GCCTTGAAGC[CG]G
ATCT[CG][CG]ATGTTTCAGGGTGAGC[CG]GA[CG]CAGG[CG
]TGCCTG[CG]CAGTG[CG][CG]GAGGAGTGCTGTT[[CG]]TG
TGTT[CG]AGTCCCTGGGTTGCTTCCTGGGGTCTGTGGTGCTGGG
TGTGCTATCTG[[CG]]TGTGATTCTCTAG[CG]AGAGATTGTGG
G[CG]AGTGAC[CG]AGTGGGCAAGGGGC[CG]TCACTGTGTGTG
[CG]TGATTTTGACAGTGTGTGGTGGTAGCTTCTGACTC[CG][C
G]TGGGT[CG]TTGAATGTATGACTGGGAC[CG]TTTAG[CG]GT
GGATACACAACTGTGTG[CG]GCTGTGGAAGATTGCC
SEQ ID NO: 5045
TCTGGGGTCTTGTAACTCTCTGAGAACCTCATGAAACAAGATTTC
CTCTCAGATTAAAAAAAAAAAAAGGCA[CG]CATAACAGTGAATG
CAAAACATC[CG]CAATTTCAGGGGGT[CG]GAGACCCCTG[[CG
]]CACCCACCTAAGGGGTCCATGCACCTGGGCTATGAGGCTGGAA
GCAGAAAGGGGG[CG]CCAGAAAG[CG]CA[CG]C[CG][CG]AG
GGC[CG]G[CG]GGAAATGTAGTCTCA[CG]C[CG]GTAAAGCCA
CAGAG[CG]A[CG]ATGAGG[CG]AGA[CG]TCTG[CG]TTCTTC
TCAGGCCTGAGACCCAAC[CG]GCC[[CG]]GGCACCAGCTCCTG
CTGGACCCCAGAG[CG]CCTGGAAAGCTC[CG]GAACCTCTCACC
TGC[CG]CCTCCT[CG]GGTCCAAGTGCCAC[CG][CG]GC[CG]
[CG]CCCC[CG]GCAGCCCAGGG[CG][CG]CTTCCACCA[CG]G
TAC[CG]GTGGATT[CG]C[CG]TG[[CG]]CAGC[CG]GAAGAT
GG[CG]CAGA[CG]CACAAAGCACAC[CG]ATGCTG[CG]CCATG
ATAGGGC[CG]G[CG]C[CG]CAGTGTCTTC[CG]GGAAACATAG
TCTTTAGG[CG]TAAAGGCAGCAGCC[[CG]]GCCTTGAAGC[CG
]GATCT[[CG]][CG]ATGTTTCAGGGTGAGC[CG]G
SEQ ID NO: 5046
GC[CG]CCAGAGCCAGAGC[CG]GAGCTG[CG]G[CG]G[CG]TG
GACC[CG]GCAGGGG[CG]GGCCT[CG]GACAGCAGAGA[CG]CA
G[CG]GGCC[CG]GC[CG]AC[CG]GGCTG[CG]GGAGTGGCCC[
CG]GGCATGGGG[CG]GC[CG]G[CG]GGGGC[CG][CG]GG[CG
]GG[CG]CTG[CG]CTT[CG]GGCTGG[CG]G[CG]GGCTG[CG]
TGAG[CG]GC[CG]GGA[CG]CAGGATG[CG]CTC[CG]AGGGTG
[CG]GCCCC[CG]GGC[CG]G[CG]G[CG]C[CG]CTGTG[CG]G
GG[CG]CTGAGCCTGCTGCTGGG[CG][CG]CTGCTGGGCAAAGG
TAAGGC[[CG]]GGG[CG]GGTGCCTGG[CG][CG]C[CG]A[CG
]CATG[CG]AACTTCC[CG]GTTC[CG]GGGGAGCT[CG]GGGTG
AGGGCAGC[CG]AAG[CG]AGGGGTG[CG]G[CG]GGGGCCT[CG
]G[CG]GGGGCT[CG]GATCCCATCAACAG[CG]CCTCTG[CG]G
GTGC[CG]AGC[CG]CCCCTAATCCAGAAGGG[CG]CTCCCAGCT
GGG[CG]C[CG]GGATC[CG]GGCAAGTTAGGCTGGTCC[CG]AG
TCC[CG]AATCTCCCACCC[CG]GACCCCCCAGACCCAGAGCTGC
CC[CG][CG]GATGAGGAGGGGG[CG]TGGCTGAGGGACA[CG]G
TGTTTGTTCCCCATCCCTGAGTTC[CG]TG[[CG]]
SEQ ID NO: 5047
ATCCCCT[CG]GATGGACTCTTGCAAACCTCCAGCCCCATCC[[C
G]]GGATC[CG]TG[CG]TCTTCTACAGCCACCACTTGGGCCAG[
CG]TCCAGGA[CG]CCCAAG[CG]TAACCTCCCCACTCTAAAC[C
G]TTAGGAATCAGTGGCTGGACTCAC[CG][CG]TCATAAAAGCA
TAAACTGTGACC[CG]ATGAGTC[CG][CG]CACT[CG]GAAAAC
AAGAGAA[CG]CTCCCA[CG]TCTC[CG]GG[CG]CAGCTTGTGC
CAG[CG]ACTACTAGGTCAGGGGG[CG]CAGCTGAGAGAATTCAA
GCAACCTCCC[CG]CAAC[CG]GTTC[CG]C[CG][[CG]]TTTG
TGGGCTGGTAGCC[CG]GAATACATTTCCCAGAGGCCTT[[CG]]
[[CG]]GC[CG]A[CG]TGCTT[CG][CG]CAGGAA[CG]CAGC[
CG]CCTCC[CG]ACTGGAGGA[CG][CG]GTAG[CG]GAGCTGCT
CAAAG[CG]AGGTGCTCAGCCCTGGGAGGCTCAGGGCTGCC[[CG
]]AGGGGCTTAGTCCCAGAGCCAGTGCTGTCAGGGGCTGAGCATG
TGAATAGGGCAGT[[CG]]GAAGACCATGTGTGTCTGTGGCTTTT
AAATACACCC[CG]AA[[CG]]TCTATCTCTTTTAACTGGAGTAT
TTAATTTATTTACATTTAATATAGC
SEQ ID NO: 5048
ATTTATAAGCTGCCATCCCTAGCCTTCCCC[CG]CTTCCCCTC[C
G]CTCCCT[CG]CCAGACTTG[CG]GG[CG]A[CG]GGCACAGC[
CG][CG]TCTGGCTCTTCC[CG]GCCTCTCCCTTCTCT[CG][CG
]GG[CG]CAGC[CG]ATCAATAGTTAACACC[CG]GCTG[CG]GA
[CG]G[CG]GCTCCATC[CG][CG]GCAATCAC[CG]TAGTGCTT
GTTTGTGGAAGC[CG]AG[CG]TG[CG]TG[CG]C[CG][CG][C
G][CG]CACCCAGTCCAG[CG][CG]GAGTGGG[CG]TCTACC[C
G]AGGAGGGGTGTCTGGGGAGGGGCTGCCCT[[CG]]TTACCCAA
ACAGTTTG[CG]CT[[CG]]CTTAACCTTGATGCAGCT[CG]AGG
CTTCCCAGTCCAGCTCAGTTCAGACAGAAAACCTGG[CG][CG][
CG][CG][CG][CG]CACACACACA[CG]CCTCCCCTGG[CG]T[
CG]C[CG]CC[CG]GC[CG]GGTCCCTGCCCTTAGGGACCAGAG[
CG]G[CG]AC[CG]CTGCACCC[CG]CAC[CG]CCTGCTGGAGGA
GCCCC[CG]GAGC[CG]GGGC[CG]AGC[CG]C[CG]G[CG]TCC
C[CG]AGTG[CG]CCCCCTGTG[CG]TGC[CG]C[CG][CG]CTG
TTGCT[CG]CAGTGTGCTGG[CG]C[CG]AGCT[CG]GTGGACA[
CG][[CG]][CG]CAGTCAGAGCTGCCTCT
SEQ ID NO: 5049
ATAAGCTGCCATCCCTAGCCTTCCCC[CG]CTTCCCCTC[CG]CT
CCCT[CG]CCAGACTTG[CG]GG[CG]A[CG]GGCACAGC[CG][
CG]TCTGGCTCTTCC[CG]GCCTCTCCCTTCTCT[CG][CG]GG[
CG]CAGC[CG]ATCAATAGTTAACACC[CG]GCTG[CG]GA[CG]
G[CG]GCTCCATC[CG][CG]GCAATCAC[CG]TAGTGCTTGTTT
GTGGAAGC[CG]AG[CG]TG[CG]TG[CG]C[CG][CG][CG][C
G]CACCCAGTCCAG[CG][CG]GAGTGGG[CG]TCTACC[CG]AG
GAGGGGTGTCTGGGGAGGGGCTGCCCT[[CG]]TTACCCAAACAG
TTTG[[CG]]CT[CG]CTTAACCTTGATGCAGCT[CG]AGGCTTC
CCAGTCCAGCTCAGTTCAGACAGAAAACCTGG[CG][CG][CG][
CG][CG][CG]CACACACACA[CG]CCTCCCCTGG[CG]T[CG]C
[CG]CC[CG]GC[CG]GGTCCCTGCCCTTAGGGACCAGAG[CG]G
[CG]AC[CG]CTGCACCC[CG]CAC[CG]CCTGCTGGAGGAGCCC
C[CG]GAGC[CG]GGGC[CG]AGC[CG]C[CG]G[CG]TCCC[CG
]AGTG[CG]CCCCCTGTG[CG]TGC[CG]C[CG][CG]CTGTTGC
T[CG]CAGTGTGCTGG[CG]C[CG]AGCT[CG]GTGGACA[CG][
[CG]][CG]CAGTCAGAGCTGCCTCT[CG]CC
SEQ ID NO: 5050
GTAACCTGTTACAACATTATTTATAAGCTGCCATCCCTAGCCTTC
CCC[CG]CTTCCCCTC[CG]CTCCCT[CG]CCAGACTTG[CG]GG
[CG]A[CG]GGCACAGC[CG][CG]TCTGGCTCTTCC[CG]GCCT
CTCCCTTCTCT[CG][CG]GG[CG]CAGC[CG]ATCAATAGTTAA
CACC[CG]GCTG[CG]GA[CG]G[CG]GCTCCATC[CG][CG]GC
AATCAC[CG]TAGTGCTTGTTTGTGGAAGC[CG]AG[[CG]]TG[
CG]TG[CG]C[CG][CG][CG][CG]CACCCAGTCCAG[CG][CG
]GAGTGGG[CG]TCTACC[CG]AGGAGGGGTGTCTGGGGAGGGGC
TGCCCT[[CG]]TTACCCAAACAGTTTG[[CG]]CT[[CG]]CTT
AACCTTGATGCAGCT[CG]AGGCTTCCCAGTCCAGCTCAGTTCAG
ACAGAAAACCTGG[CG][CG][CG][CG][CG][CG]CACACACA
CA[CG]CCTCCCCTGG[CG]T[CG]C[CG]CC[CG]GC[CG]GGT
CCCTGCCCTTAGGGACCAGAG[CG]G[CG]AC[CG]CTGCACCC[
CG]CAC[CG]CCTGCTGGAGGAGCCCC[CG]GAGC[CG]GGGC[C
G]AGC[CG]C[CG]G[CG]TCCC[CG]AGTG[CG]CCCCCTGTG[
CG]TGC[CG]C[CG][CG]CTGTTGCT[CG]CAGTGTGCTGG[CG
]C[CG]AGCT[CG]GTGGACA[CG][CG]C
SEQ ID NO: 5051
CCCAAGTTCTCTCTGTGTCCCCCT[CG][CG]GGCCC[CG]CACC
T[CG][CG]TCC[CG]GAT[CG]CTCTGATTC[CG][CG]ACTCC
TTGGC[CG]C[CG]CTG[CG]CATGGAAAGCTCTGCCAAGATGGA
GAG[CG]G[CG]G[CG]C[CG]GCCAGCAGCCCCAGC[CG]CAGC
CCCAGCAGCCCTTCCTGC[CG]CC[CG]CAGCCTGTTTCTTTGCC
A[CG]GC[CG]CAGC[CG][CG]G[CG]GC[CG]CAGC[CG]C[C
G]CAG[CG]GCAG[CG]CAGAG[CG][CG]CAGCAGCAGCAGCAG
CAGCAGCAGCAGCAGCAGCAGG[CG]C[CG]CAGCTGAGAC[CG]
G[CG]GC[CG]A[CG]GCCAGCCCTCAGGGGG[CG]GTCACAAGT
CAG[CG]CCCAAGCAAGTCAAG[CG]ACAG[CG]CT[CG]TCTT[
CG]CC[CG]AACTGATG[CG]CTGCAAA[CG]C[CG]GCTCAACT
TCAG[CG]GCTTTGGCTACAGCCTGC[CG]CAGCAGCAGC[CG]G
C[CG]C[CG]TGG[CG][CG]C[CG]CAA[CG]AG[CG][CG]AG
[CG]CAAC[CG][CG]TCAAGTTGGTCAACCTGGGCTTTGCCACC
CTT[CG]GGAGCA[CG]TCCCCAA[CG]G[CG][CG]GCCAACAA
GAAGATGAGTAAGGTGGAGACACTG[CG]CT[CG]G[CG]GT[CG
]AGTACATC[CG][CG][CG]CTGCA
SEQ ID NO: 5052
C[CG]CACCT[CG][CG]TCC[CG]GAT[CG]CTCTGATTC[CG]
[[CG]]ACTCCTTGGC[CG]C[CG]CTG[CG]CATGGAAAGCTCT
GCCAAGATGGAGAG[CG]G[CG]G[CG]C[CG]GCCAGCAGCCCC
AGC[CG]CAGCCCCAGCAGCCCTTCCTGC[CG]CC[CG]CAGCCT
GTTTCTTTGCCA[CG]GC[CG]CAGC[CG][CG]G[CG]GC[CG]
CAGC[CG]C[CG]CAG[CG]GCAG[CG]CAGAG[CG][CG]CAGC
AGCAGCAGCAGCAGCAGCAGCAGCAGCAGCAGG[CG]C[CG]CAG
CTGAGAC[CG]G[CG]GC[CG]A[CG]GCCAGCCCTCAGGGGG[C
G]GTCACAAGTCAG[CG]CCCAAGCAAGTCAAG[CG]ACAG[CG]
CT[CG]TCTT[CG]CC[CG]AACTGATG[CG]CTGCAAA[CG]C[
CG]GCTCAACTTCAG[CG]GCTTTGGCTACAGCCTGC[CG]CAGC
AGCAGC[CG]GC[CG]C[CG]TGG[CG][CG]C[CG]CAA[CG]A
G[CG][CG]AG[CG]CAAC[CG][CG]TCAAGTTGGTCAACCTGG
GCTTTGCCACCCTT[CG]GGAGCA[[CG]]TCCCCAA[CG]G[CG
][CG]GCCAACAAGAAGATGAGTAAGGTGGAGACACTG[CG]CT[
CG]G[CG]GT[CG]AGTACATC[CG][CG][CG]CTGCAGCAGCT
GCTGGA[CG]AGCATGA[CG][CG]GTGAG
SEQ ID NO: 5053
AGCTGGATGAAGCCCACCC[CG]TCCCCTTCTGGTACCAAAGTGC
TTACTCCTCTCCAAAGTGC[CG]TGTCTGAACTGC[CG]CTGGGA
AGAAG[CG]GCTCCTGAGA[CG][CG]CCCACACCTTTCACCTGC
[CG][CG][CG]CTTCCCCCTCCT[CG]GCCACCTTCC[CG]G[C
G]GAAGCAG[CG]AGGAGGGAGCCCCCTTTGGC[CG]TCCTC[CG
]TGGAAC[CG]GTTTTC[CG]AGGCTGGCAAAAGC[CG]AGGCTG
GATTTGGGGGAGGAATATTAGACT[CG]GAGGAGTCTG[CG][CG
]CTTTTCTCCTCCC[CG][CG]CCTCC[CG]GT[CG]C[[CG]][
CG]GGTTCAC[CG]CTCAGTCCC[CG][CG]CT[CG]CTC[CG]C
ACCCCACCCACTTCCTGTGCT[CG]CC[CG]GGGGG[CG]TGTGC
[CG]TG[CG]GCTGC[CG]GAGTT[CG]GGGAAGTTGTGGCTGT[
CG]AGAATGGGGGTCTGTGGGTACCTGTTCCTGCCCTGGAAGTGC
CT[CG]TGGT[CG]TGTCTCTCAGGCTGCTGTTCCTTGTACCCAC
AGGAGTGCC[CG]TG[CG]CAG[CG]GAGATGCCACCTTCCCCAA
AGCTATGGACAA[CG]TGA[CG]GTC[CG]GCAGGGGGAGAG[CG
]CCACCCTCAGGTAGGGAGCTGACA
SEQ ID NO: 5054
TGGATGAAGCCCACCC[CG]TCCCCTTCTGGTACCAAAGTGCTTA
CTCCTCTCCAAAGTGC[CG]TGTCTGAACTGC[CG]CTGGGAAGA
AG[CG]GCTCCTGAGA[CG][CG]CCCACACCTTTCACCTGC[CG
][CG][CG]CTTCCCCCTCCT[CG]GCCACCTTCC[CG]G[CG]G
AAGCAG[CG]AGGAGGGAGCCCCCTTTGGC[CG]TCCTC[CG]TG
GAAC[CG]GTTTTC[CG]AGGCTGGCAAAAGC[CG]AGGCTGGAT
TTGGGGGAGGAATATTAGACT[CG]GAGGAGTCTG[CG][CG]CT
TTTCTCCTCCC[CG][CG]CCTCC[CG]GT[[CG]]C[[CG]][C
G]GGTTCAC[CG]CTCAGTCCC[CG][CG]CT[CG]CTC[CG]CA
CCCCACCCACTTCCTGTGCT[CG]CC[CG]GGGGG[CG]TGTGC[
CG]TG[CG]GCTGC[CG]GAGTT[CG]GGGAAGTTGTGGCTGT[C
G]AGAATGGGGGTCTGTGGGTACCTGTTCCTGCCCTGGAAGTGCC
T[CG]TGGT[CG]TGTCTCTCAGGCTGCTGTTCCTTGTACCCACA
GGAGTGCC[CG]TG[CG]CAG[CG]GAGATGCCACCTTCCCCAAA
GCTATGGACAA[CG]TGA[CG]GTC[CG]GCAGGGGGAGAG[CG]
CCACCCTCAGGTAGGGAGCTGACATTG
SEQ ID NO: 5055
CAAAAGAAAGGGGGTGGGCAGAAAGGGAGATTTCCATCCCTTCAA
A[CG]TGCA[CG]CCCCTCC[CG][CG]GTCC[CG]GGAGGGAGT
CCCCCCTCCCAGGT[CG]AG[CG][CG]TCCAC[CG]GCTGTCCC
CACAGCAACAGG[CG]GG[CG]G[CG]GGGGAGGAGAGGC[CG][
CG]GGGGCAG[CG]CAGGCCTGG[CG]G[CG]GC[CG]AAATCCC
C[CG]GCCACCA[CG]A[CG]TTCCC[CG]GCTCTG[CG]G[CG]
[CG]C[CG]GGAGCAGGTGGTTGCCCTCTG[CG]TCCAGGG[CG]
TGTACTGAGCCCAGGCTAGCTGAG[CG]AGGCTGCAGCCCCTG[[
CG]]ATGGCC[CG]GGAAGAGGCAAGTGC[CG][CG]GGGCCAGA
G[CG]AGGAGGGACCAGCAGGG[CG]GCCTGGACCCCCACTTGGC
[CG][CG]CAGGCAGGAGGGG[CG][CG]CCCTCTG[CG]GAG[C
G][CG]G[CG]C[CG][CG]GCAGC[CG]CAGCATCTGG[CG]CT
[CG]CTCTCTCTGGC[CG]GAGAGGC[CG][CG]ACACCTAG[CG
][CG]GGGC[CG]AGGCTAGGAAGGG[CG]GGC[CG]GGGAGGGA
GGAGGGCACTGGGAGGAAGC[CG][CG]GG[[CG]]AGGAAGCCC
[CG]CTCCTGC[CG]AGTGCCTGGAGAGGAGATTCCCAGCCTTCC
TGCCCA
SEQ ID NO: 5056
AGCC[CG][CG]CTCACTCCTGCCCTCCT[CG]CAAAGAACTGGC
CAGCCCCACT[CG][CG]TCCAGCCCACAACTAGGCTGC[CG]GG
AC[CG]GGGAGCCAGACCCCAGGT[CG]CTCCAACTC[CG]G[CG
]AGGG[CG]GGGAGGGGAGTGACAGCAGCAACTAG[CG][CG][C
G]CTGGCAAAAGCACAGAGTCT[[CG]]GAATAGGCTAAA[CG]G
C[CG]G[CG][CG]GAGGGGTGGAGGCAAGAGGGGG[CG]GAGGA
GGAGTTGCC[CG]AG[CG]GAGAGGGATGA[CG]TCCTCCAGGGC
CTTGGGCTC[CG]AGGAAC[CG]AAGACTGGTG[CG]CT[CG]C[
CG]GGCCC[CG][CG]CACTAGCTCTGGGGTT[CG]CAGAGG[CG
]C[CG][CG]CC[CG]GCTTC[CG][CG]GCAGCTCCAACACAG[
CG][CG]CAGG[CG]GT[CG]ATCC[CG]GTCAGCTTAAAC[CG]
GG[CG]CAAGCCCTCTGTCCCCCTCAAAGCACCTCTCTGCT[CG]
TCCCTAGTCTCCAGGTGCC[CG]TCTGAGAGTCCCTGT[CG]CCT
C[CG]GG[CG]GACC[CG]GCCAGGCAGCCCC[CG]GT[CG]GGC
CAGCCT[CG]CCCCTTCCT[CG]CTTTCTCCCCCAT[CG]ACCT[
CG]AGCCAAGGGCAGG[CG]GCC[CG]CACC[CG][CG]CCCCTG
CC
SEQ ID NO: 5057
[CG]CCTCTGGGTAGCTGGATGAAGCCCACCC[CG]TCCCCTTCT
GGTACCAAAGTGCTTACTCCTCTCCAAAGTGC[CG]TGTCTGAAC
TGC[CG]CTGGGAAGAAG[CG]GCTCCTGAGA[CG][CG]CCCAC
ACCTTTCACCTGC[CG][CG][CG]CTTCCCCCTCCT[CG]GCCA
CCTTCC[CG]G[CG]GAAGCAG[CG]AGGAGGGAGCCCCCTTTGG
C[CG]TCCTC[CG]TGGAAC[CG]GTTTTC[CG]AGGCTGGCAAA
AGC[CG]AGGCTGGATTTGGGGGAGGAATATTAGACT[CG]GAGG
AGTCTG[CG][CG]CTTTTCTCCTCCC[CG][[CG]]CCTCC[CG
]GT[[CG]]C[[CG]][CG]GGTTCAC[CG]CTCAGTCCC[CG][
CG]CT[CG]CTC[CG]CACCCCACCCACTTCCTGTGCT[CG]CC[
CG]GGGGG[CG]TGTGC[CG]TG[CG]GCTGC[CG]GAGTT[CG]
GGGAAGTTGTGGCTGT[CG]AGAATGGGGGTCTGTGGGTACCTGT
TCCTGCCCTGGAAGTGCCT[CG]TGGT[CG]TGTCTCTCAGGCTG
CTGTTCCTTGTACCCACAGGAGTGCC[CG]TG[CG]CAG[CG]GA
GATGCCACCTTCCCCAAAGCTATGGACAA[CG]TGA[CG]GTC[C
G]GCAGGGGGAGAG[CG]CCACCCTCAGGTA
SEQ ID NO: 5058
AA[CG]GCCTGGGTC[CG]GCTTCCTCCTCCCCTGAAGCAG[CG]
AAGGGG[CG]TCACTGG[CG][CG]CTGTTAAA[CG]TCTT[CG]
CCAT[CG]GTTCCCTGCATCCTCCCTCCTGCCTGGGTAGCCAGCT
CCTGGG[CG][CG]CCCAGG[CG]CACA[CG]CTTCCCT[CG]CT
CCTCCTGCAGCCACAA[CG]CCCCCACCCCCCC[CG]CC[CG]CC
[CG]CCTTGTG[CG]GCAG[CG]GTGAC[CG]CCTTCTCCTGCTG
CCACCCCCTCCACCCCCTGTTCAAGGTCTGTCAC[CG]TAGGGGG
[CG]GGGGGG[CG][CG]TGGAGC[CG]CTGGGGGTT[[CG]]GC
CCACCC[CG][CG]AAC[CG]AGCTCC[CG]GCCCTGTG[CG]CC
CTCAGCTCTGC[CG][CG]GG[CG]TTGGCCTGGG[CG]GG[CG]
CAGGACTGCCTGGT[CG]CCCTGGAGCTCAGGGT[CG]GGGCCC[
CG][CG]TTTAACCACCAGG[CG]GAGG[CG]CAGG[CG]GAGCC
[CG]GAGAGGA[CG][CG]ACCCCTCTGGG[CG]GCCTTGGGCCC
CTGGA[CG]TGCCAGACTCTGGACCCCTG[CG]GGGGCTGC[CG]
TGGAGAGCAGAGGCAC[CG][CG]TCCTGCCCAGAGCTC[CG]AT
AGG[CG]GGC[CG][CG]TTGGGGGATTGTGGGGGC[CG]G
SEQ ID NO: 5059
GAGGCAAGTTATATAAAAG[CG]GC[[CG]]GCCCCCCTGCCCAG
[CG]GGGATGCTTTGTG[CG]GAG[CG]CACCTTAT[CG]GCCC[
CG][CG]CTG[CG]CC[CG]CC[CG][CG]CCTGCACC[CG]GGA
[CG]AGCC[CG]CTGC[CG]C[CG][CG][CG]CTGAG[CG]CAG
GGCCCCTCC[CG][CG]GCC[CG]CCAGC[CG]CCACC[CG]CC[
CG]GG[CG]G[CG]C[CG]CAGTGT[CG]CCCCCTGTCCCC[CG]
CACCTCCC[CG]GGGGAGGGGC[CG]GCC[CG][CG]CT[CG]CC
CCCAGCC[CG]AGGGGCAACTTAAAACATGG[CG]CCC[CG]GG[
CG]GGGGATTTGTGCAAATTGG[[CG]]GAGAAGGGA[CG][CG]
GGGAC[CG]AGT[CG]CCCCTT[CG]GCCAGGGATCCCAGGGAGG
CCCCCAGGC[CG]GAGGC[CG]GGGCTCAGGCTCTG[CG][CG]C
[CG]GCCCAGCCACTACTG[CG]C[CG][CG]G[CG]GG[CG]GA
G[CG]GG[CG]GGGGG[CG][CG]G[CG][CG]CAGGCT[CG]GC
C[CG]GTGGGGGTCC[CG]G[CG]AG[CG]GGAGGG[CG]GTTGG
GGACCC[CG]GC[CG][CG]C[CG]GG[CG][CG]GGGCT[CG]G
GATT[CG]GGAGAC[CG][CG][CG]G[CG]C[CG]AAGCCA[CG
][[CG]]TCAGCCCCACTGTCC[CG][CG][CG]CCT[CG]CCCC
AGGCCT[CG]GGCTCTT
SEQ ID NO: 5060
CTCCAGGCACTCTCAAGGATC[CG]GTTCATGTCTT[CG]CCCTG
GCC[CG]CTTGGTATAGGGGG[CG]CACAGGAAGAGGCCAAGAAG
TGGAAAGAACCTCATCT[CG]ATGTCCCAAGTGACAAAGCCCC[C
G]GGGCT[CG]GGGCTTTGGTACAGAGGAGCAGCAGAAGC[CG]C
[CG]C[CG]CAGA[CG]CC[CG]GATG[CG]GAGGACCCAGAG[C
G]CTG[CG]CAGCTGAGGAGCACCTGGGGAGCCAC[CG]C[CG]G
[CG]CCAACTTTTGCCATCC[CG]GGAGCCATAG[CG]GCAGAGA
GGGGGTCC[CG]GACC[CG]AGGTGGCAG[CG]AGGGGCTCTGCT
TTGGCAGCTGT[CG]GGGCTGGAGCTCAGGACCCCAACTCCAGCC
AAGT[CG]GGC[CG][CG]G[CG]GGGCAGG[CG]TAGGCTG[CG
]CAGGCAGGGGTGGC[CG][CG]GGTGCTGAGCCAGT[CG]C[CG
]C[CG][CG]CAGG[CG]GG[CG]CTGGAGTCTCAGCTACCTGG[
CG][CG]AC[CG]CCAGCAGCACCCAG[CG][CG]GGGC[CG]GG
AGCTGCTGGGGGCCCAGGCTC[CG]CTCTCCCCAC[CG]CTCTGC
AC[CG]CTGC[CG]GCTG[CG]GACAGACC[CG]ATG[CG]CCAC
CACCAC[CG]CAGTTGCCA[CG]ACCCCCAT
SEQ ID NO: 5061
GAAAGCTCTGCCAAGATGGAGAG[CG]G[CG]G[CG]C[CG]GCC
AGCAGCCCCAGC[CG]CAGCCCCAGCAGCCCTTCCTGC[CG]CC[
CG]CAGCCTGTTTCTTTGCCA[CG]GC[CG]CAGC[CG][CG]G[
CG]GC[CG]CAGC[CG]C[CG]CAG[CG]GCAG[CG]CAGAG[CG
][CG]CAGCAGCAGCAGCAGCAGCAGCAGCAGCAGCAGCAGG[CG
]C[CG]CAGCTGAGAC[CG]G[CG]GC[CG]A[[CG]]GCCAGCC
CTCAGGGGG[CG]GTCACAAGTCAG[CG]CCCAAGCAAGTCAAG[
CG]ACAG[CG]CT[CG]TCTT[CG]CC[CG]AACTGATG[CG]CT
GCAAA[CG]C[[CG]]GCTCAACTTCAG[CG]GCTTTGGCTACAG
CCTGC[CG]CAGCAGCAGC[CG]GC[CG]C[CG]TGG[CG][CG]
C[CG]CAA[CG]AG[CG][CG]AG[CG]CAAC[CG][CG]TCAAG
TTGGTCAACCTGGGCTTTGCCACCCTT[CG]GGAGCA[[CG]]TC
CCCAA[CG]G[CG][CG]GCCAACAAGAAGATGAGTAAGGTGGAG
ACACTG[CG]CT[CG]G[CG]GT[CG]AGTACATC[CG][CG][C
G]CTGCAGCAGCTGCTGGA[CG]AGCATGA[CG][CG]GTGAG[C
G]C[CG]CCTTCCAGGCAGG[CG]TCCTGT[CG]CCCACCATCTC
CCCCAACTACTCCAA[CG]ACTTG
SEQ ID NO: 5062
CTTCTTGCCT[CG]CCAC[CG][CG]TGCTCCTGGG[CG][CG]C
CC[CG]C[CG][CG]GGCCCTTGAGG[CG][CG][CG]GAGACAC
CAG[CG]CTGGCTTCC[CG]GGCC[CG][CG]GGC[CG]GGGAGG
GAAGCCT[CG]GGGCTG[CG]GGGTGAGAGGAAGAAAGCAAACC[
CG]GGGAGCAGG[CG]GCTGC[CG]CACC[CG][CG]CACCC[CG
]GGCCCTCACCA[CG]CCCTCCC[CG][CG][CG]C[CG]GCTCA
GGGGCTGCCC[CG]GAATCAGCTCCC[CG]GGGC[CG]C[CG]CA
A[CG]AAGGTGGATC[CG]CATCTTGATTGTTCTC[CG]GGAGCC
TCCTGGGGGCTC[[CG]]G[CG]G[CG]G[CG][CG]GG[CG][C
G]ACCCATCC[CG]CTGG[CG]CTCCC[CG]CT[CG]CTGAACCC
[CG]TTTGCCTGTCCACACCCCCT[CG]CTCCCCACCATTTTTCC
TGAC[CG]GCCTGTGTCCC[CG]AGCCCT[CG][CG]GCAGGCC[
CG]AGCAGG[CG]AT[CG][CG]GC[CG]GGCA[CG][CG][CG]
CCC[CG]GGCTCC[CG]CCCCCCTTC[CG]AGCATC[CG]C[CG]
CCTCTTTTCTGCTGGGTCTGGGAGGAGGGAGGCTGGGAGGC[CG]
CT[CG]GGGCCCAG[CG]TGCCAGCCC[CG]GAGTTCAGCCTCC[
CG]AGCTG[CG]G[CG]CC[[CG]]CAG[CG]GA
SEQ ID NO: 5063
AGCCACTACTG[CG]C[CG][CG]G[CG]GG[CG]GAG[CG]GG[
CG]GGGGG[CG][CG]G[CG][CG]CAGGCT[CG]GCC[CG]GTG
GGGGTCC[CG]G[CG]AG[CG]GGAGGG[CG]GTTGGGGACCC[C
G]GC[CG][CG]C[CG]GG[CG][CG]GGGCT[CG]GGATT[CG]
GGAGAC[CG][CG][CG]G[CG]C[CG]AAGCCA[CG][CG]TCA
GCCCCACTGTCC[CG][CG][CG]CCT[CG]CCCCAGGCCT[CG]
GGCTCTTCCTC[CG]CACCT[CG]TAAAGC[CG]AGACCCCCT[[
CG]]CAGTCCCCCACTC[CG]AGAGG[CG]GAAAAGTTACCTGGG
ATCAGCAGGGAGCC[CG]GG[CG][CG]C[CG][CG]G[[CG]]T
GGGGACTAGGCT[CG]GG[CG][CG][CG]TCCT[CG]G[CG]G[
CG]GTG[CG]CAGGAGACT[CG]GG[CG]TGGGGAGGAAGC[CG]
CAGCCCAGGGCTGCT[[CG]]C[CG]CTGTTCCCCC[CG]CCCCC
TGTTGCAGGAGACAC[CG]AGGCTC[CG][CG]GAGCTG[CG]G[
CG]GGGGCCCA[[CG]]CCCAGAGAC[CG]TG[CG]AGGAAAGCC
AACCT[CG]TCCTGCCTGCCCTCCCAGC[CG][[CG]]GGGGAGG
GGGCCAC[[CG]]CAGTACTTCAGGACAGTTACATGGGCACAGCC
TCCTC[CG]TCCTGG[CG]CAGGGTCAGGCTC[CG][CG]GA[CG
]A[CG]CCT
SEQ ID NO: 5064
[CG]GTTGGGGACCC[CG]GC[CG][CG]C[CG]GG[CG][CG]G
GGCT[CG]GGATT[CG]GGAGAC[CG][CG][CG]G[CG]C[CG]
AAGCCA[CG][[CG]]TCAGCCCCACTGTCC[CG][CG][CG]CC
T[CG]CCCCAGGCCT[CG]GGCTCTTCCTC[CG]CACCT[CG]TA
AAGC[CG]AGACCCCCT[[CG]]CAGTCCCCCACTC[CG]AGAGG
[CG]GAAAAGTTACCTGGGATCAGCAGGGAGCC[CG]GG[CG][C
G]C[CG][CG]G[CG]TGGGGACTAGGCT[CG]GG[CG][CG][C
G]TCCT[CG]G[CG]G[CG]GTG[CG]CAGGAGACT[CG]GG[CG
]TGGGGAGGAAGC[CG]CAGCCCAGGGCTGCT[[CG]]C[CG]CT
GTTCCCCC[CG]CCCCCTGTTGCAGGAGACAC[CG]AGGCTC[CG
][CG]GAGCTG[CG]G[[CG]]GGGGCCCA[[CG]]CCCAGAGAC
[CG]TG[CG]AGGAAAGCCAACCT[CG]TCCTGCCTGCCCTCCCA
GC[CG][[CG]]GGGGAGGGGGCCAC[[CG]]CAGTACTTCAGGA
CAGTTACATGGGCACAGCCTCCTC[[CG]]TCCTGG[CG]CAGGG
TCAGGCTC[[CG]][CG]GA[[CG]]A[CG]CCTGGAGACAGCTG
CCAATGCCAATAGCTTTAGCCCTTTATTCCCACTTAGATGATGGC
CTGGCCTCTCCAGACCCCTCTCCCCTCCCA
SEQ ID NO: 5065
[CG][CG]TCAGCCCCACTGTCC[CG][CG][CG]CCT[CG]CCC
CAGGCCT[CG]GGCTCTTCCTC[CG]CACCT[CG]TAAAGC[CG]
AGACCCCCT[[CG]]CAGTCCCCCACTC[CG]AGAGG[CG]GAAA
AGTTACCTGGGATCAGCAGGGAGCC[CG]GG[CG][CG]C[CG][
CG]G[[CG]]TGGGGACTAGGCT[CG]GG[CG][CG][CG]TCCT
[CG]G[CG]G[CG]GTG[CG]CAGGAGACT[CG]GG[CG]TGGGG
AGGAAGC[CG]CAGCCCAGGGCTGCT[[CG]]C[CG]CTGTTCCC
CC[CG]CCCCCTGTTGCAGGAGACAC[CG]AGGCTC[CG][CG]G
AGCTG[CG]G[CG]GGGGCCCA[[CG]]CCCAGAGAC[CG]TG[C
G]AGGAAAGCCAACCT[CG]TCCTGCCTGCCCTCCCAGC[CG][C
G]GGGGAGGGGGCCAC[CG]CAGTACTTCAGGACAGTTACATGGG
CACAGCCTCCTC[[CG]]TCCTGG[CG]CAGGGTCAGGCTC[CG]
[CG]GA[CG]A[CG]CCTGGAGACAGCTGCCAATGCCAATAGCTT
TAGCCCTTTATTCCCACTTAGATGATGGCCTGGCCTCTCCAGACC
CCTCTCCCCTCCCAGACATCCCCTGTGAGAAAGGAGGGAGAGGGT
GTTCCTGACCTTCAGGTGAGTTATTTATTGGCTAGGG
SEQ ID NO: 5066
GC[CG]AAGCCA[CG][CG]TCAGCCCCACTGTCC[CG][CG][C
G]CCT[CG]CCCCAGGCCT[CG]GGCTCTTCCTC[CG]CACCT[C
G]TAAAGC[CG]AGACCCCCT[CG]CAGTCCCCCACTC[CG]AGA
GG[CG]GAAAAGTTACCTGGGATCAGCAGGGAGCC[CG]GG[CG]
[CG]C[CG][CG]G[[CG]]TGGGGACTAGGCT[CG]GG[CG][C
G][CG]TCCT[CG]G[CG]G[CG]GTG[CG]CAGGAGACT[CG]G
G[CG]TGGGGAGGAAGC[CG]CAGCCCAGGGCTGCT[[CG]]C[C
G]CTGTTCCCCC[CG]CCCCCTGTTGCAGGAGACAC[CG]AGGCT
C[CG][CG]GAGCTG[CG]G[CG]GGGGCCCA[[CG]]CCCAGAG
AC[CG]TG[CG]AGGAAAGCCAACCT[CG]TCCTGCCTGCCCTCC
CAGC[CG][[CG]]GGGGAGGGGGCCAC[[CG]]CAGTACTTCAG
GACAGTTACATGGGCACAGCCTCCTC[[CG]]TCCTGG[CG]CAG
GGTCAGGCTC[[CG]][CG]GA[[CG]]A[CG]CCTGGAGACAGC
TGCCAATGCCAATAGCTTTAGCCCTTTATTCCCACTTAGATGATG
GCCTGGCCTCTCCAGACCCCTCTCCCCTCCCAGACATCCCCTGTG
AGAAAGGAGGGAGAGGGTGTTCCTGACCTTCAGGTGAGTTATTTA
SEQ ID NO: 5067
AG[CG]GCAG[CG]CAGAG[CG][CG]CAGCAGCAGCAGCAGCAG
CAGCAGCAGCAGCAGCAGG[CG]C[CG]CAGCTGAGAC[CG]G[C
G]GC[CG]A[CG]GCCAGCCCTCAGGGGG[CG]GTCACAAGTCAG
[CG]CCCAAGCAAGTCAAG[CG]ACAG[CG]CT[CG]TCTT[CG]
CC[CG]AACTGATG[CG]CTGCAAA[CG]C[CG]GCTCAACTTCA
G[CG]GCTTTGGCTACAGCCTGC[CG]CAGCAGCAGC[CG]GC[C
G]C[CG]TGG[CG][CG]C[CG]CAA[CG]AG[CG][CG]AG[CG
]CAAC[CG][CG]TCAAGTTGGTCAACCTGGGCTTTGCCACCCTT
[CG]GGAGCA[CG]TCCCCAA[CG]G[CG][CG]GCCAACAAGAA
GATGAGTAAGGTGGAGACACTG[CG]CT[CG]G[CG]GT[CG]AG
TACATC[CG][CG][CG]CTGCAGCAGCTGCTGGA[CG]AGCATG
A[CG][CG]GTGAG[CG]C[CG]CCTTCCAGGCAGG[CG]TCCTG
T[CG]CCCACCATCTCCCCCAACTACTCCAA[CG]ACTTGAACTC
CATGGC[CG]GCT[CG]C[CG]GTCTCATCCTACT[CG]T[CG]G
A[CG]AGGGCTCTTA[CG]ACC[CG]CTCAGCCC[CG]AGGAGCA
GGAGCTTCT[CG]ACTTCACCAACTGGTTCTGAGGGGCT[CG]GC
CTGGTCAGGC
SEQ ID NO: 5068
GCTCACCAGCCTATCTTGCCTAAGAATCACAAGGTCA[CG]ATAG
[CG]T[CG][CG]ATTCCACTCAC[CG]TACACTCCTTGGCCAGA
GATCCCCTCCAGGAGGACTGTCAGCAGCCACA[CG]AGAC[CG]T
ACAGTAAATCCATCTTCA[CG]TGAACATGAACATATCCAGCCCA
GGGGGTA[[CG]]CAA[CG]GGACCTTCAGGAGGC[CG]G[CG]G
CCAGCCAGG[CG][CG]CTCCACT[CG][CG]CC[CG]GGCCAAG
C[CG]AGGTGCC[CG]GGAAC[CG]CT[CG]C[CG]AAGGAGGAA
G[CG]C[CG]TC[CG]TCTGTCCTTCCC[CG]G[CG]G[CG]G[C
G]G[CG]AG[CG]GAG[CG]CAGGAGCCC[CG]CACTCCACACAC
TCATGCACACACACACTCACACACACTCACACACTCTCCCACAAC
ACAATACCCTGACACACACTCACA[CG]CA[CG]C[CG]CACTCA
CAC[CG]GGTGCCTGGGAAAAT[CG]CAGA[CG]C[CG]GGGAGG
AGCAGGGGG[CG]GTGATGGGAAGGGGAGCTG[CG]AGG[CG]AA
GTGTTCTTCAGGGAAG[CG]GGCT[CG]AGTCTC[[CG]]CAGCT
G[CG]G[CG]G[CG]G[CG]G[CG]G[CG][CG]CTGGGC[CG]G
[CG]G[CG]GG[CG][CG]GGCAGGGGGC[CG]GGGGTGC[CG][
CG][CG]G
SEQ ID NO: 5069
CTATCCTCCACCCC[CG]GGGGGTTCCTG[CG]CACTGAAAGAC[
CG]TTCTC[CG]GCAGGTTTTGGGATC[CG]G[CG]A[CG]GCTG
AC[CG][CG][CG][CG]CCCCCA[CG]CC[CG]GTTCCA[CG]A
TGCTGCAATACAGAAAGTTTA[CG]T[CG]GCCC[CG]ACC[CG]
[CG][CG]GGACTGCAGGGTC[CG]C[CG]GAG[CG][CG]G[CG
]CAGAGGCTTTTCCTG[CG][CG]TT[CG]GCCC[CG]GGAAAGG
GG[CG]GGAGGGCTGGCTC[CG]GGAG[CG]CA[CG]GG[CG][C
G]G[CG]GGGAGGGTACTCACTGTGAAGCA[CG]CTG[CG]CCCA
TGGATCATGTCTGTG[[CG]]TTACACCAGAGGCTC[CG]GGCTC
CACTAATTCCATTTAGAGA[CG]GGAAGACTTCCAGTGG[CG]GG
GGGAGGACAGGGT[CG]AGAGGTGTTAAAGA[CG]CAAAGCAAGA
AGGAAATAAAGGGGGGC[CG]AGAGGGAGAC[CG]AGAGGAAGGG
GGAGCTC[CG]AGCCCA[CG]CTGCAGCCAGATC[CG]GATGAGT
C[CG]TCCTC[CG]CCC[CG]GG[CG]GGCTCT[CG]CTCT[CG]
CTGGCCCTCAG[CG]C[CG][CG]CAGCCAGCAGCATCCCCAC[C
G]TGA[CG]CT[CG]CATCACACC[CG]GG[CG]C[CG]GC[CG]
CCACCATC[CG]C
SEQ ID NO: 5070
TGGGGGCTC[CG]G[CG]G[CG]G[CG][CG]GG[CG][CG]ACC
CATCC[CG]CTGG[CG]CTCCC[CG]CT[CG]CTGAACCC[CG]T
TTGCCTGTCCACACCCCCT[CG]CTCCCCACCATTTTTCCTGAC[
CG]GCCTGTGTCCC[CG]AGCCCT[CG][CG]GCAGGCC[CG]AG
CAGG[CG]AT[CG][CG]GC[CG]GGCA[CG][CG][CG]CCC[C
G]GGCTCC[CG]CCCCCCTTC[CG]AGCATC[CG]C[CG]CCTCT
TTTCTGCTGGGTCTGGGAGGAGGGAGGCTGGGAGGC[CG]CT[CG
]GGGCCCAG[CG]TGCCAGCCC[CG]GAGTTCAGCCTCC[CG]AG
CTG[CG]G[CG]CC[[CG]]CAG[CG]GAGGAGGTTTTCAGTGGC
TGATTGAAACTCACTGCAAAATCACCA[CG]ACTCTTTCACCTAC
TGAGATGATTGAC[CG]AGGTTTGGCCTTCCATTTTTACTGAGAT
TTGG[CG]AGAC[CG]AATGGAAG[CG]TC[CG]CACAGTAACTG
CAGCTGCTAGGCCAGAGGGGCCC[CG]G[CG]CCCTTCC[CG]CC
TCCCCTCC[CG]CTTGCTTTTGCCTTACT[CG]ATCTTACCACCA
CCCCTCCCC[CG]GCCCCC[CG]ACTGAGAACT[CG]GGCCTCTC
ACC[CG]CCCCCCAGCCTCC[CG]CTCTGGG[CG]AGCC
SEQ ID NO: 5071
[CG]CAGCC[CG]GTGGGGCAACCCTGACT[CG]GAC[CG]CT[C
G]GGAGAGCCCCAGGAGAGGCCAG[CG]C[CG][CG]CAGCAGC[
[CG]]CCC[CG]CTG[CG]CCCACCTCCC[CG]GCTGCTCC[CG]
GAGGGCTCACAAAGG[CG]GTGGC[CG]CC[CG]AGTGGCCTTCT
CCATCCAGG[[CG]]TT[CG][CG]TCCTCCTCCCCACCTTCTCT
CC[CG]AAGG[CG]AAAATGGCAGGGCCAGG[CG]AGAACCTGGG
ACAG[CG]GTGGCCCTAGCCCTG[[CG]]ATCCTCACCCCTCCTG
CTAGGAGAGGCTG[CG]GGCTGCC[CG][CG]GA[CG]ATGTGGC
[CG][CG]GCTGCTCC[CG]AG[CG]CATCTT[CG]GCC[CG]GG
TCCC[CG]C[CG]CCACCCCTCTTCTCTGCTCCTTCCATCC[CG]
CCCAGAGGAGTTGATGC[CG]CTGT[CG]C[CG]C[CG]C[CG]C
[CG]CTGCTGAAGC[CG][CG]GCTGATGGATGCCAGGGAGTGC[
CG]CATTGCTTAG[CG]ACCC[CG]CCTCTGGGTTTGCTGGTAGG
AG[CG]GCTGCTCTTTCTTCTTTCTTGCTTTGGGGTTTTATAGAA
AAGATAAGGACATTTTTATTTTATTCTTCACAA[CG]TCCTCCCC
TTCTCTT[CG]TTTTTGAAATGTGCATTCCCAG
SEQ ID NO: 5072
[CG]GTCTCCAGGG[CG]GAAAGGGGTGGAAGCTGACCCT[CG]C
CCTTCCCTCCAG[CG]CTGGCTTCAGGTGTGCCTTCTGCTACCTC
CTGTACTG[CG]AACAGGGGCC[CG]C[CG]AGCTC[CG]GGAGC
CCCTAGAAGAGGAAGACTCCTCTGGCCCCACTAGGTATCATC[CG
][CG]CTCTCC[CG]CTTTCCACCTG[CG]CCCT[CG]CTTGGGC
CAATCTCTGC[CG]CA[CG]TGTCCATCCCTGAACTGCA[CG]CT
ATCCTCCACCCC[CG]GGGGGTTCCTG[CG]CACTGAAAGAC[CG
]TTCTC[CG]GCAGGTTTTGGGATC[[CG]]G[CG]A[CG]GCTG
AC[CG][CG][CG]C[CG]CCCCCA[CG]CC[CG]GTTCCA[CG]
ATGCTGCAATACAGAAAGTTTA[CG]T[CG]GCCC[CG]ACC[CG
][CG][CG]GGACTGCAGGGTC[CG]C[CG]GAG[CG][CG]G[C
G]CAGAGGCTTTTCCTG[CG][CG]TT[CG]GCCC[CG]GGAAAG
GGG[CG]GGAGGGCTGGCTC[CG]GGAG[CG]CA[CG]GG[CG][
CG]G[CG]GGGAGGGTACTCACTGTGAAGCA[CG]CTG[CG]CCC
ATGGATCATGTCTGTG[[CG]]TTACACCAGAGGCTC[CG]GGCT
CCACTAATTCCATTTAGAGA[CG]GGAAGACTTCCAGTGG[CG]G
SEQ ID NO: 5073
AG[CG]GC[CG]TCCTTCCCCAGCT[CG]AAGCC[CG]TAGCACA
TT[CG]CAGGCAAAGCCTCCCAAGT[CG]TCTAGGCAGTTAGGGA
GCTCTG[CG]CATTTGCCAGCA[CG]GAGGTACCTCC[CG]GGGC
AGGGACACAACACAT[CG]CC[CG]AGAGTTTGTCCCAG[CG]AG
[CG]C[CG]ATTT[CG]TC[CG][CG]ATGCAAGTAACTGAGAT[
CG]GGAGCTGTCCC[CG]GCAGAG[CG]CACTCACCT[CG]GTCC
CAGGTGGACTGAAGTCCAGAG[CG]G[CG]CTGTGCAGCTGGAAG
GG[CG][CG][CG]ATAGCTCAAGTTAGAGG[CG]GCCC[CG]GG
G[CG][CG]G[CG]CAGGACACAAGACCTCAAACTGGTACTTGCA
CAGGTAGC[CG]TTGG[CG][CG]CAGGTGGCAT[CG]CATCTCC
TTCCAGCCTG[CG]GGCT[CG]ACCCCAC[CG]GTGGCCTGGAGT
AC[CG][CG]CATCTC[CG][CG][CG]GTGCAGGAG[CG]TTGG
GGCTCCTCCACCCACTGCAG[CG]TGT[CG]CTTT[CG]AGAC[C
G]C[CG]GGGT[CG]GAGGACAGCCAGGAGAAACCC[CG]CAAAG
GCT[CG]TTCTCCAGGGTGCAGTGGGAA[CG]CCTG[CG]CTCCA
GTG[[CG]]ACCCAGAACAGCAGGTCTTTGGAGCCCC
SEQ ID NO: 5074
GCTGTCCTTCCCCACTGGTCACACAAGAG[CG]GC[CG]TCCTTC
CCCAGCT[CG]AAGCC[CG]TAGCACATT[CG]CAGGCAAAGCCT
CCCAAGT[CG]TCTAGGCAGTTAGGGAGCTCTG[CG]CATTTGCC
AGCA[CG]GAGGTACCTCC[CG]GGGCAGGGACACAACACAT[CG
]CC[CG]AGAGTTTGTCCCAG[CG]AG[CG]C[CG]ATTT[CG]T
C[CG][CG]ATGCAAGTAACTGAGAT[CG]GGAGCTGTCCC[CG]
GCAGAG[CG]CACTCACCT[CG]GTCCCAGGTGGACTGAAGTCCA
GAG[CG]G[CG]CTGTGCAGCTGGAAGGG[[CG]][CG][CG]AT
AGCTCAAGTTAGAGG[CG]GCCC[[CG]]GGG[CG][CG]G[CG]
CAGGACACAAGACCTCAAACTGGTACTTGCACAGGTAGC[CG]TT
GG[CG][CG]CAGGTGGCAT[CG]CATCTCCTTCCAGCCTG[CG]
GGCT[CG]ACCCCAC[CG]GTGGCCTGGAGTAC[CG][CG]CATC
TC[CG][CG][CG]GTGCAGGAG[CG]TTGGGGCTCCTCCACCCA
CTGCAG[CG]TGT[CG]CTTT[CG]AGAC[CG]C[CG]GGGT[CG
]GAGGACAGCCAGGAGAAACCC[CG]CAAAGGCT[CG]TTCTCCA
GGGTGCAGTGGGAA[CG]CCTG[CG]CTCCAGTG[[CG]]A
SEQ ID NO: 5075
[CG]GCTGTCCTTCCCCACTGGTCACACAAGAG[CG]GC[CG]TC
CTTCCCCAGCT[CG]AAGCC[CG]TAGCACATT[CG]CAGGCAAA
GCCTCCCAAGT[CG]TCTAGGCAGTTAGGGAGCTCTG[CG]CATT
TGCCAGCA[CG]GAGGTACCTCC[CG]GGGCAGGGACACAACACA
T[CG]CC[CG]AGAGTTTGTCCCAG[CG]AG[CG]C[CG]ATTT[
CG]TC[CG][CG]ATGCAAGTAACTGAGAT[CG]GGAGCTGTCCC
[CG]GCAGAG[CG]CACTCACCT[CG]GTCCCAGGTGGACTGAAG
TCCAGAG[CG]G[CG]CTGTGCAGCTGGAAGGG[[CG]][[CG]]
[CG]ATAGCTCAAGTTAGAGG[CG]GCCC[[CG]]GGG[CG][CG
]G[CG]CAGGACACAAGACCTCAAACTGGTACTTGCACAGGTAGC
[CG]TTGG[CG][CG]CAGGTGGCAT[CG]CATCTCCTTCCAGCC
TG[CG]GGCT[CG]ACCCCAC[CG]GTGGCCTGGAGTAC[CG][C
G]CATCTC[CG][CG][CG]GTGCAGGAG[CG]TTGGGGCTCCTC
CACCCACTGCAG[CG]TGT[CG]CTTT[CG]AGAC[CG]C[CG]G
GGT[CG]GAGGACAGCCAGGAGAAACCC[CG]CAAAGGCT[CG]T
TCTCCAGGGTGCAGTGGGAA[CG]CCTG[CG]CTCCAGTG[[CG]
]
SEQ ID NO: 5076
G[[CG]][[CG]][CG]ATAGCTCAAGTTAGAGG[CG]GCCC[CG
]GGG[CG][CG]G[CG]CAGGACACAAGACCTCAAACTGGTACTT
GCACAGGTAGC[CG]TTGG[CG][CG]CAGGTGGCAT[CG]CATC
TCCTTCCAGCCTG[CG]GGCT[CG]ACCCCAC[CG]GTGGCCTGG
AGTAC[CG][CG]CATCTC[CG][CG][CG]GTGCAGGAG[CG]T
TGGGGCTCCTCCACCCACTGCAG[CG]TGT[CG]CTTT[CG]AGA
C[CG]C[CG]GGGT[CG]GAGGACAGCCAGGAGAAACCC[CG]CA
AAGGCT[CG]TTCTCCAGGGTGCAGTGGGAA[CG]CCTG[CG]CT
CCAGTG[CG]ACCCAGAACAGCAGGTCTTTGGAGCCCCCTC[CG]
GGCCCTGGGCCTGCC[CG]CAGGAG[CG][CG]AGCACAG[CG][
CG]CAGCT[CG]G[CG]CC[CG]CA[CG]CA[CG]GTGCTGAG[C
G]CCCCACCT[CG]CAGGATGCAGGCCTCCT[CG]GC[CG]CCTG
C[CG]CTTCATGGTAG[CG]TGGTGCAGGCTGTAGCAGGCCCC[C
G]AGGC[CG]AGCAGCCAGCA[CG]GT[CG]GCAGTGGGGTGTT[
CG]C[CG]C[CG]CC[CG]GCC[CG]GGCCAGAG[CG]CCTGCCA
GAGGAGGCACAGGG[CG]AA[CG]C[CG]GCCTCATTCTCTGAGG
CCC[CG]CAC
SEQ ID NO: 5077
CCCCCCTCTCCTTTGCACCCCATTCCTGC[CG]CTCCCACCTCCC
CTTCCCAGTCC[CG]CCAGGTGACTGTGTCTCATGCAG[CG]AAG
GCAACAAG[CG]TCCCCAGC[CG]AGGAAGGGAAGGGGCAGC[CG
]TCTTGGCT[CG][CG]AGGGAAGGAGGG[CG]GAAG[CG]GG[C
G]CC[CG]CT[[CG]]CAAGCTTATTGCATCCTAC[CG]CCTGTA
GAGCTGGCCTCCTCCAGCTGAGC[CG]AAATGCTTTCCACCCTCC
TCACATTCATGTCTAGGGA[CG]CAGGGTGCAGAAG[CG]AGACT
[CG]AGAGTCCAC[CG]GCCAGGGG[[CG]]TCTGTCCA[CG]GG
TCTGCA[CG]GGAG[CG]CAC[CG]C[CG]CT[CG]GCC[CG]GG
GG[CG]TC[CG]TGG[CG]CTGGGAG[CG]AGAGCTT[CG]G[CG
]G[CG]GCTG[CG]GGCAGGG[CG][CG][CG]GAG[CG]GGCTG
CAGGTG[CG]GG[CG]GAGGC[CG]G[CG]GGGGG[CG]C[CG]G
GGG[CG]CTG[CG][CG]GCCCTTGG[CG]G[CG]GGGG[CG][C
G][CG]GGGTGG[CG]GGGGAGGC[CG]AGG[CG]C[CG]GCAGC
TT[CG][CG]C[CG]G[CG]GCTGGAAG[CG]GG[CG]GGCTGCA
[CG]GG[CG]GCT[CG]AGTG[[CG]]GGGACCCCAGCCCCT[CG
]CCCT[CG]TGAG[CG]C[CG]CCCCTG
SEQ ID NO: 5078
AAGGAGAGAAGTGGT[CG]GTGTCTGTTTCCTTCTGTCCCC[CG]
GGGC[CG]TGGAGCTGT[CG]GAGGGAAGGAGGA[CG]GTG[CG]
GGGC[CG]CAGGGGG[CG][CG]GGG[CG][CG]G[CG]GGACCC
AGGCTA[CG]AG[CG]GGAGGGAGG[CG]GGAGT[CG]GGGGAAG
A[CG][CG]GCAGGC[CG]GC[CG]AGGGCACCC[CG]AGGAACA
TGGCATGGCCTCTGTG[CG]ATC[CG]AGT[CG][CG]GTCTC[C
G]GGGTGCCTGGGAGGGC[CG]AACCACTGGTGAGGG[CG]TGGG
GAGCAGGGGGTGGCAGAGGGCACC[CG]GG[CG]GTAGTC[CG]G
GA[CG][CG]CAAGGCAGAGCCCTGA[CG]CTC[CG]GGTCCC[C
G]TGCCTGGCTCTTCTTGCCT[CG]CCAC[CG][CG]TGCTCCTG
GG[CG][CG]CCC[CG]C[CG][CG]GGCCCTTGAGG[CG][CG]
[CG]GAGACACCAG[CG]CTGGCTTCC[CG]GGCC[CG][CG]GG
C[CG]GGGAGGGAAGCCT[CG]GGGCTG[CG]GGGTGAGAGGAAG
AAAGCAAACC[CG]GGGAGCAGG[CG]GCTGC[CG]CACC[CG][
CG]CACCC[CG]GGCCCTCACCA[CG]CCCTCCC[CG][CG][CG
]C[CG]GCTCAGGGGCTGCCC[CG]GAATCAGCTCCC[CG]GGGC
[CG]C[CG]CAA[CG]AAGG
SEQ ID NO: 5079
CTT[CG]GCCAGGGATCCCAGGGAGGCCCCCAGGC[CG]GAGGC[
CG]GGGCTCAGGCTCTG[CG][CG]C[CG]GCCCAGCCACTACTG
[CG]C[CG][CG]G[CG]GG[CG]GAG[CG]GG[CG]GGGGG[CG
][CG]G[CG][CG]CAGGCT[CG]GCC[CG]GTGGGGGTCC[CG]
G[CG]AG[CG]GGAGGG[CG]GTTGGGGACCC[CG]GC[CG][CG
]C[CG]GG[CG][CG]GGGCT[CG]GGATT[CG]GGAGAC[CG][
CG][CG]G[CG]C[CG]AAGCCA[CG][[CG]]TCAGCCCCACTG
TCC[CG][CG][CG]CCT[CG]CCCCAGGCCT[CG]GGCTCTTCC
TC[CG]CACCT[CG]TAAAGC[CG]AGACCCCCT[CG]CAGTCCC
CCACTC[CG]AGAGG[CG]GAAAAGTTACCTGGGATCAGCAGGGA
GCC[CG]GG[CG][CG]C[CG][CG]G[CG]TGGGGACTAGGCT[
CG]GG[CG][CG][CG]TCCT[CG]G[CG]G[CG]GTG[CG]CAG
GAGACT[CG]GG[CG]TGGGGAGGAAGC[CG]CAGCCCAGGGCTG
CT[CG]C[CG]CTGTTCCCCC[CG]CCCCCTGTTGCAGGAGACAC
[CG]AGGCTC[CG][CG]GAGCTG[CG]G[[CG]]GGGGCCCA[C
G]CCCAGAGAC[CG]TG[CG]AGGAAAGCCAACCT[CG]TCCTGC
CTGCCCTCCCAGC[CG][[CG]]GGGGAGGGGGCCAC[[CG]]CA
GTACT
SEQ ID NO: 5080
AGACCCCCT[CG]CAGTCCCCCACTC[CG]AGAGG[CG]GAAAAG
TTACCTGGGATCAGCAGGGAGCC[CG]GG[CG][CG]C[CG][CG
]G[CG]TGGGGACTAGGCT[CG]GG[CG][CG][CG]TCCT[CG]
G[CG]G[CG]GTG[CG]CAGGAGACT[CG]GG[CG]TGGGGAGGA
AGC[CG]CAGCCCAGGGCTGCT[CG]C[CG]CTGTTCCCCC[CG]
CCCCCTGTTGCAGGAGACAC[CG]AGGCTC[CG][CG]GAGCTG[
CG]G[CG]GGGGCCCA[CG]CCCAGAGAC[CG]TG[CG]AGGAAA
GCCAACCT[CG]TCCTGCCTGCCCTCCCAGC[CG][[CG]]GGGG
AGGGGGCCAC[CG]CAGTACTTCAGGACAGTTACATGGGCACAGC
CTCCTC[[CG]]TCCTGG[CG]CAGGGTCAGGCTC[[CG]][CG]
GA[[CG]]A[CG]CCTGGAGACAGCTGCCAATGCCAATAGCTTTA
GCCCTTTATTCCCACTTAGATGATGGCCTGGCCTCTCCAGACCCC
TCTCCCCTCCCAGACATCCCCTGTGAGAAAGGAGGGAGAGGGTGT
TCCTGACCTTCAGGTGAGTTATTTATTGGCTAGGGACTGCAGTAT
TTTTTTTTTCTTTTTGGAAAGAAAATAAAATCAAGTTGGTGGTGT
CTTCAAGTCATGTTA
SEQ ID NO: 5081
C[CG][CG]GGCTTGAACCCTTTCCATCTTTCATCTGTTTACACA
CAGCCCCAATACCTTTTCTCTAATTGCTCTCCCTTTTTAATTTAG
TTTTATTTTTAAACAGACCATTAGACCATGGCTTGTTTCTGGGCT
GAGCCTCC[CG]CTGGTGCAGCCCTGCAGATGGTC[[CG]]CTTC
AGCTACTC[CG]TTAGGAGG[CG]GGCAGTTTTGTCTGTGTCTTA
CTCCTTC[CG]TCCAGGGCATTC[CG]CAGACTCCTGCTCCCTTC
TTCCTCTCCACTTGCTCTCAATAGAGGG[CG][CG]CTGGG[CG]
AAGTAGGGG[[CG]]TGGCAGGAGCCC[CG]CTC[CG][CG]GAT
CCAGGTTGAAATTCCTCCCTCCTGGAATC[CG]AC[CG]TGAGCA
TTTCTGAG[CG]GTGGTGAGGAGGAGGCCACCAAGACACCTCCCT
CC[CG]CTGTGCAAAGGAA[CG]CAGGAGCC[CG]G[CG]CAGGT
GGTGGGCTTAC[CG][CG][CG]CA[CG]CCTGGCTGGAGAGGTG
A[CG]C[CG]CTGTCTGCCAACCTTTCCCAGCTTTTCCCA[CG]A
TTGGAGCCAAGAGAGGTCCC[[CG]]GGGATCTCAGTTGGGCAAT
TGTAAGGGAAGGAGCTCAGGACACTGACTTCAAGAT
SEQ ID NO: 5082
CTGGGTTCCCC[CG]AGGAAAAATACCCACC[CG][CG]AGGGCT
[CG]G[CG]GCTTTT[CG]ACT[CG]G[CG]GGGATGAACTGTGG
CAACTT[[CG]]GCAGCCCCCAC[CG][CG]GTG[CG]GAAGTAA
AGAGGGCAACATTGG[CG]ACTG[CG]GCT[CG]GAGGGGCTGGA
G[CG][CG]TGAAGC[CG]TGGGGG[[CG]]C[CG]TG[CG]CCT
CC[CG]CTCTCT[CG]TTT[CG]GC[CG]CAGGTCCTGGGACTC[
CG]ACTT[CG]GTGCTC[CG]GGTGATAG[CG]GCTG[CG]G[CG
]CCTGCAGTCCAGATCCT[CG]CAGTTCTG[CG]GG[CG]AAGGA
GG[[CG]]AA[CG]GAAT[CG]GCCCCCAGTGGGGAG[CG]CAAC
AAGCCACAGTAGCCAAACCC[CG][CG]CTCCTGCC[CG]GGCTC
CCAGA[CG]AA[CG]CAGC[CG]CAG[CG]GGGGGC[CG]GGCCC
[CG]AGCCCCA[CG]C[CG]C[CG]C[CG]C[CG][CG]CCAG[C
G]GTGGGGGG[CG]GGCTGGGGG[CG]GGG[CG]GC[CG]GGGCT
GCCTTCC[CG]GG[CG]CATATG[CG]AG[CG]CAGCACC[CG]G
[CG]CTGC[CG]AGCCACCTCCCC[CG]C[CG]CC[CG]CTAGCA
AGTTTGG[[CG]]GCTCCAAGCCAGG[CG][CG]CCTCAGGATCC
AGGCTCATTTGCTTCCACCTAGCTT[CG]GTG
SEQ ID NO: 5083
TCAGCAGCCTGCTGAGGAGTGGGGA[[CG]]AAGAGCAGCCTAAA
CTTAGGGCT[CG]GGATATTT[CG]ATGCCACCCAAATTGC[CG]
TCCTACCCCAA[CG]AGGCAGGGAAAGGAG[CG]GAG[CG][CG]
[CG][CG][CG]AGCTGAGTGAGTGCTTA[CG]T[CG]CAG[CG]
AGATCTGTGCTGGGATAATTAGAGAGGAGTTGGGCTGAGC[CG]A
GTCCTCTTTCAGCAGCAGCAGC[CG]GAGC[CG]C[CG]C[CG]C
AGCC[CG]GTGGGGCAACCCTGACT[CG]GAC[CG]CT[CG]GGA
GAGCCCCAGGAGAGGCCAG[CG]C[CG][CG]CAGCAGC[[CG]]
CCC[CG]CTG[CG]CCCACCTCCC[CG]GCTGCTCC[CG]GAGGG
CTCACAAAGG[CG]GTGGC[CG]CC[CG]AGTGGCCTTCTCCATC
CAGG[CG]TT[CG][CG]TCCTCCTCCCCACCTTCTCTCC[CG]A
AGG[CG]AAAATGGCAGGGCCAGG[CG]AGAACCTGGGACAG[[C
G]]GTGGCCCTAGCCCTG[[CG]]ATCCTCACCCCTCCTGCTAGG
AGAGGCTG[CG]GGCTGCC[CG][CG]GA[CG]ATGTGGC[CG][
CG]GCTGCTCC[CG]AG[CG]CATCTT[CG]GCC[CG]GGTCCC[
CG]C[CG]CCACCCCTCTTCTCTGCTCCTTCCATCC[CG]CCC
SEQ ID NO: 5084
C[CG]CCT[CG]GTCTGGGTTCCCC[CG]AGGAAAAATACCCACC
[CG][CG]AGGGCT[CG]G[CG]GCTTTT[CG]ACT[CG]G[CG]
GGGATGAACTGTGGCAACTT[[CG]]GCAGCCCCCAC[CG][CG]
GTG[CG]GAAGTAAAGAGGGCAACATTGG[CG]ACTG[CG]GCT[
CG]GAGGGGCTGGAG[CG][CG]TGAAGC[CG]TGGGGG[[CG]]
C[CG]TG[CG]CCTCC[CG]CTCTCT[CG]TTT[CG]GC[CG]CA
GGTCCTGGGACTC[CG]ACTT[CG]GTGCTC[CG]GGTGATAG[C
G]GCTG[CG]G[CG]CCTGCAGTCCAGATCCT[CG]CAGTTCTG[
CG]GG[CG]AAGGAGG[[CG]]AA[CG]GAAT[[CG]]GCCCCCA
GTGGGGAG[CG]CAACAAGCCACAGTAGCCAAACCC[CG][CG]C
TCCTGCC[CG]GGCTCCCAGA[CG]AA[CG]CAGC[CG]CAG[CG
]GGGGGC[CG]GGCCC[CG]AGCCCCA[CG]C[CG]C[CG]C[CG
]C[CG][CG]CCAG[CG]GTGGGGGG[CG]GGCTGGGGG[CG]GG
G[CG]GC[CG]GGGCTGCCTTCC[CG]GG[CG]CATATG[CG]AG
[CG]CAGCACC[CG]G[CG]CTGC[CG]AGCCACCTCCCC[CG]C
[CG]CC[CG]CTAGCAAGTTTGG[[CG]]GCTCCAAGCCAGG[CG
][CG]CCTCAGGATCCAGGCTCATTTGCTTCCACCT
SEQ ID NO: 5085
AAAATACCCACC[CG][CG]AGGGCT[CG]G[CG]GCTTTT[CG]
ACT[CG]G[CG]GGGATGAACTGTGGCAACTT[CG]GCAGCCCCC
AC[CG][CG]GTG[CG]GAAGTAAAGAGGGCAACATTGG[CG]AC
TG[CG]GCT[CG]GAGGGGCTGGAG[CG][CG]TGAAGC[CG]TG
GGGG[[CG]]C[CG]TG[CG]CCTCC[CG]CTCTCT[CG]TTT[C
G]GC[CG]CAGGTCCTGGGACTC[CG]ACTT[CG]GTGCTC[CG]
GGTGATAG[CG]GCTG[CG]G[CG]CCTGCAGTCCAGATCCT[CG
]CAGTTCTG[CG]GG[CG]AAGGAGG[[CG]]AA[[CG]]GAAT[
[CG]]GCCCCCAGTGGGGAG[[CG]]CAACAAGCCACAGTAGCCA
AACCC[CG][CG]CTCCTGCC[CG]GGCTCCCAGA[CG]AA[CG]
CAGC[CG]CAG[CG]GGGGGC[CG]GGCCC[CG]AGCCCCA[CG]
C[CG]C[CG]C[CG]C[CG][CG]CCAG[CG]GTGGGGGG[CG]G
GCTGGGGG[CG]GGG[CG]GC[CG]GGGCTGCCTTCC[CG]GG[C
G]CATATG[CG]AG[CG]CAGCACC[CG]G[CG]CTGC[CG]AGC
CACCTCCCC[CG]C[[CG]]CC[CG]CTAGCAAGTTTGG[CG]GC
TCCAAGCCAGG[CG][CG]CCTCAGGATCCAGGCTCATTTGCTTC
CACCTAGCTT[CG]GTGCCCCCTGCTAGG[CG]GGG
SEQ ID NO: 5086
CT[CG]GTCTGGGTTCCCC[CG]AGGAAAAATACCCACC[CG][C
G]AGGGCT[CG]G[CG]GCTTTT[CG]ACT[CG]G[CG]GGGATG
AACTGTGGCAACTT[CG]GCAGCCCCCAC[CG][CG]GTG[CG]G
AAGTAAAGAGGGCAACATTGG[CG]ACTG[CG]GCT[CG]GAGGG
GCTGGAG[CG][CG]TGAAGC[CG]TGGGGG[CG]C[CG]TG[CG
]CCTCC[CG]CTCTCT[CG]TTT[CG]GC[CG]CAGGTCCTGGGA
CTC[CG]ACTT[CG]GTGCTC[CG]GGTGATAG[CG]GCTG[CG]
G[CG]CCTGCAGTCCAGATCCT[CG]CAGTTCTG[CG]GG[CG]A
AGGAGG[[CG]]AA[[CG]]GAAT[[CG]]GCCCCCAGTGGGGAG
[[CG]]CAACAAGCCACAGTAGCCAAACCC[CG][CG]CTCCTGC
C[CG]GGCTCCCAGA[CG]AA[CG]CAGC[CG]CAG[CG]GGGGG
C[CG]GGCCC[CG]AGCCCCA[CG]C[CG]C[CG]C[CG]C[CG]
[CG]CCAG[CG]GTGGGGGG[CG]GGCTGGGGG[CG]GGG[CG]G
C[CG]GGGCTGCCTTCC[CG]GG[CG]CATATG[CG]AG[CG]CA
GCACC[CG]G[CG]CTGC[CG]AGCCACCTCCCC[CG]C[CG]CC
[CG]CTAGCAAGTTTGG[[CG]]GCTCCAAGCCAGG[CG][CG]C
CTCAGGATCCAGGCTCATTTGCTTCCACCTAGCT
SEQ ID NO: 5087
ACCT[CG][CG]CACCAGCAGC[CG]GCCAG[CG]G[CG]GCAGC
AGCCCATGCCTC[CG]GTGCAACAGCTG[CG]CCTCCTC[CG]GT
GCCC[CG]G[CG]G[CG]GGGG[CG]GGAGATAACCTGTCCCTGC
TGCTC[CG]CACCTCCT[CG]CC[CG]G[CG]G[CG]CCTTC[CG
]GACC[CG]CACCTCCT[CG]C[CG]CTGT[CG]GGCT[CG]TCC
TGCTGCTGCTGCTGCTGCT[CG]T[CG][CG]C[CG]GGGCAGCC
AGCTCAATGTGAG[CG]AGCTGA[CG]C[CG]TCCAGCCATGCCA
GTG[CG]CTC[CG]GCAGCAGTA[CG][CG]CAGCAGTC[CG][C
G]CAGCAGT[CG]G[CG]TC[CG]CCTCCCAGTACCACCAGTGCC
ACAGCCTGCAGCC[CG]C[CG]CCAGCCCCA[CG]GGCAGCCT[C
G]GCAGTCTGGGCTC[CG]GGCCCC[CG]CTCT[CG]CACCACCA
CCACCACC[CG]CACC[CG]G[CG]CACCACCAGCACCACCAGCC
CCAGG[CG][CG]C[CG][CG]AGAGCAACCCCTTCAC[CG]AAA
TAGCCATGAGCAGCTGCAGGTACAA[CG]GGGG[CG]TCATG[[C
G]]GC[CG]CTCAGCAACTTGAG[CG][CG]TCC[CG]C[CG]GA
ACCTGCA[CG]AGATGGACTCAGAGG[CG]CAGCCCCTGCAGCCC
CC[CG][CG]TCTG
SEQ ID NO: 5088
ACT[[CG]][[CG]][CG]TCTC[CG]G[CG]TGACTGC[CG]AG
ATTGA[[CG]]TGGAGGACA[CG]TCAAATTGATTCC[CG]CA[C
G]CTGCAGCCTCC[CG]GTCAGA[CG]AATTTCTCCCAAT[CG]G
ATGAAGTTCACCCTGGGCCTGGGGT[[CG]][CG]GG[CG]TGGA
GAGTGTCCTGGGAGGGGGCAGCAG[CG]G[CG]G[CG]GCAGGCC
CTGGAG[CG]GG[CG]GCAG[CG][CG]CTC[CG]CTGC[CG][C
G]CACAG[CG][CG]TCTCCAGCC[CG][CG]GCTGGGC[CG]C[
CG][CG]GCTCT[CG]GCTCT[CG]GG[CG]CCCTCCCTCTATGC
CTCTCA[CG][CG]G[CG]G[CG]G[CG]G[CG]CCCAAGCTCTC
C[CG]GACTG[CG]C[CG]GGCCCAGCCC[CG]GCCACCC[CG]G
CCACCC[CG]G[CG]CCAGGCAGCTGGC[CG]GCC[CG]CT[CG]
CTATGGGTAAGGGG[CG]GG[CG]ACAGGCAGGGCT[CG]GGAGG
GCTGATGAGGTGGGT[CG]GGTC[[CG]]CCTGCCTCCCTTCCCT
CT[CG]TC[[CG]]CTTTCCCTGGG[[CG]]ACCCAGTCCTGGGT
CCTGGAGAAGCTGCATTCTCCTCTGATCCCCAGCCTTCAGGGGGC
AGTTGAGCAAGTCTGGGAAGGGAGTGTTCCAAGTAGA[CG][CG]
CTAAAAGT[CG][CG]AAAGGG
SEQ ID NO: 5089
TC[[CG]]CTTCAGCTACTC[CG]TTAGGAGG[CG]GGCAGTTTT
GTCTGTGTCTTACTCCTTC[CG]TCCAGGGCATTC[CG]CAGACT
CCTGCTCCCTTCTTCCTCTCCACTTGCTCTCAATAGAGGG[CG][
CG]CTGGG[CG]AAGTAGGGG[[CG]]TGGCAGGAGCCC[CG]CT
C[CG][CG]GATCCAGGTTGAAATTCCTCCCTCCTGGAATC[CG]
AC[CG]TGAGCATTTCTGAG[CG]GTGGTGAGGAGGAGGCCACCA
AGACACCTCCCTCC[CG]CTGTGCAAAGGAA[CG]CAGGAGCC[C
G]G[CG]CAGGTGGTGGGCTTAC[CG][CG][CG]CA[CG]CCTG
GCTGGAGAGGTGA[CG]C[CG]CTGTCTGCCAACCTTTCCCAGCT
TTTCCCA[CG]ATTGGAGCCAAGAGAGGTCCC[CG]GGGATCTCA
GTTGGGCAATTGTAAGGGAAGGAGCTCAGGACACTGACTTCAAGA
TGAAGGGGGAGGTCAG[CG]AAGACAGTTCTAGGGTGGG[CG]G[
CG]GC[CG]CTGACAGAGCAGGAGCCACAGCCAC[CG]GGGCTTG
GAGATAGGAGCAAGT[CG]CAGG[CG]GAGGGGGCTG[CG]GGCT
GGCTGCCTGGGCAGCACAGGACTTGAGGGAGCTG[[CG]]GGGAC
TCC
SEQ ID NO: 5090
TCTGTGCTGGGATAATTAGAGAGGAGTTGGGCTGAGC[CG]AGTC
CTCTTTCAGCAGCAGCAGC[CG]GAGC[CG]C[CG]C[CG]CAGC
C[CG]GTGGGGCAACCCTGACT[CG]GAC[CG]CT[CG]GGAGAG
CCCCAGGAGAGGCCAG[CG]C[CG][CG]CAGCAGC[CG]CCC[C
G]CTG[CG]CCCACCTCCC[CG]GCTGCTCC[CG]GAGGGCTCAC
AAAGG[CG]GTGGC[CG]CC[CG]AGTGGCCTTCTCCATCCAGG[
CG]TT[CG][CG]TCCTCCTCCCCACCTTCTCTCC[CG]AAGG[C
G]AAAATGGCAGGGCCAGG[CG]AGAACCTGGGACAG[[CG]]GT
GGCCCTAGCCCTG[[CG]]ATCCTCACCCCTCCTGCTAGGAGAGG
CTG[CG]GGCTGCC[CG][CG]GA[CG]ATGTGGC[CG][[CG]]
GCTGCTCC[CG]AG[CG]CATCTT[CG]GCC[CG]GGTCCC[CG]
C[CG]CCACCCCTCTTCTCTGCTCCTTCCATCC[CG]CCCAGAGG
AGTTGATGC[CG]CTGT[CG]C[CG]C[CG]C[CG]C[CG]CTGC
TGAAGC[CG][CG]GCTGATGGATGCCAGGGAGTGC[CG]CATTG
CTTAG[CG]ACCC[CG]CCTCTGGGTTTGCTGGTAGGAG[CG]GC
TGCTCTTTCTTCTTTCTTGCTTTGGGGTTTTATAGAA
SEQ ID NO: 5091
AGAGAGGAGTTGGGCTGAGC[CG]AGTCCTCTTTCAGCAGCAGCA
GC[CG]GAGC[[CG]]C[CG]C[CG]CAGCC[CG]GTGGGGCAAC
CCTGACT[CG]GAC[CG]CT[CG]GGAGAGCCCCAGGAGAGGCCA
G[CG]C[CG][CG]CAGCAGC[[CG]]CCC[CG]CTG[CG]CCCA
CCTCCC[CG]GCTGCTCC[CG]GAGGGCTCACAAAGG[CG]GTGG
C[CG]CC[CG]AGTGGCCTTCTCCATCCAGG[[CG]]TT[CG][C
G]TCCTCCTCCCCACCTTCTCTCC[CG]AAGG[CG]AAAATGGCA
GGGCCAGG[CG]AGAACCTGGGACAG[CG]GTGGCCCTAGCCCTG
[CG]ATCCTCACCCCTCCTGCTAGGAGAGGCTG[CG]GGCTGCC[
CG][CG]GA[CG]ATGTGGC[CG][CG]GCTGCTCC[CG]AG[CG
]CATCTT[CG]GCC[CG]GGTCCC[CG]C[CG]CCACCCCTCTTC
TCTGCTCCTTCCATCC[CG]CCCAGAGGAGTTGATGC[CG]CTGT
[CG]C[CG]C[CG]C[CG]C[CG]CTGCTGAAGC[CG][CG]GCT
GATGGATGCCAGGGAGTGC[CG]CATTGCTTAG[CG]ACCC[[CG
]]CCTCTGGGTTTGCTGGTAGGAG[CG]GCTGCTCTTTCTTCTTT
CTTGCTTTGGGGTTTTATAGAAAAGATAAGGACATTTTT
SEQ ID NO: 5092
CTACCCCAA[CG]AGGCAGGGAAAGGAG[CG]GAG[CG][CG][C
G][CG][CG]AGCTGAGTGAGTGCTTA[CG]T[CG]CAG[CG]AG
ATCTGTGCTGGGATAATTAGAGAGGAGTTGGGCTGAGC[CG]AGT
CCTCTTTCAGCAGCAGCAGC[CG]GAGC[CG]C[CG]C[CG]CAG
CC[CG]GTGGGGCAACCCTGACT[CG]GAC[CG]CT[CG]GGAGA
GCCCCAGGAGAGGCCAG[CG]C[CG][CG]CAGCAGC[[CG]]CC
C[CG]CTG[CG]CCCACCTCCC[CG]GCTGCTCC[CG]GAGGGCT
CACAAAGG[CG]GTGGC[CG]CC[CG]AGTGGCCTTCTCCATCCA
GG[[CG]]TT[CG][CG]TCCTCCTCCCCACCTTCTCTCC[CG]A
AGG[CG]AAAATGGCAGGGCCAGG[CG]AGAACCTGGGACAG[CG
]GTGGCCCTAGCCCTG[[CG]]ATCCTCACCCCTCCTGCTAGGAG
AGGCTG[CG]GGCTGCC[CG][CG]GA[CG]ATGTGGC[CG][[C
G]]GCTGCTCC[CG]AG[CG]CATCTT[CG]GCC[CG]GGTCCC[
CG]C[CG]CCACCCCTCTTCTCTGCTCCTTCCATCC[CG]CCCAG
AGGAGTTGATGC[CG]CTGT[CG]C[CG]C[CG]C[CG]C[CG]C
TGCTGAAGC[CG][CG]GCTGATGGATGCCAGGGAGTGC[CG]CA
TTGCTTAG
SEQ ID NO: 5093
AGGGTGCAGAAG[CG]AGACT[CG]AGAGTCCAC[CG]GCCAGGG
G[CG]TCTGTCCA[CG]GGTCTGCA[CG]GGAG[CG]CAC[CG]C
[CG]CT[CG]GCC[CG]GGGG[CG]TC[CG]TGG[CG]CTGGGAG
[CG]AGAGCTT[CG]G[CG]G[CG]GCTG[CG]GGCAGGG[CG][
CG][CG]GAG[CG]GGCTGCAGGTG[CG]GG[CG]GAGGC[CG]G
[CG]GGGGG[CG]C[CG]GGGG[CG]CTG[CG][CG]GCCCTTGG
[CG]G[CG]GGGG[CG][CG][CG]GGGTGG[CG]GGGGAGGC[C
G]AGG[CG]C[CG]GCAGCTT[CG][CG]C[CG]G[CG]GCTGGA
AG[CG]GG[CG]GGCTGCA[CG]GG[CG]GCT[CG]AGTG[[CG]
]GGGACCCCAGCCCCT[[CG]]CCCT[CG]TGAG[CG]C[CG]CC
CCTGCCACCTGCTGCCAAGTCAC[CG]GACTGGGGATG[CG]GGG
GG[CG]GGGTG[CG]GGGAGAGGAAAG[CG]C[CG][CG]GGTGG
CA[CG]GTGCCTCTGG[CG]ACAGAGTAGATGGACC[CG]AAAGA
GACC[CG]AGGTGACTT[CG]GGCTGGGCAGGACACCCC[CG]GG
AGTTGCCAG[CG]GCTCTCAG[CG]GG[CG]CCTCTAGGACCCCT
TGTCACTG[CG]AGCAG[CG]CCTGCTCTGGGGAGGGGCT[CG]A
AGG[CG]C[CG][CG]CCTTCTCAA[CG][CG]C[CG]CTG[[CG
]]T
SEQ ID NO: 5094
CT[CG]AGAGTCCAC[CG]GCCAGGGG[CG]TCTGTCCA[CG]GG
TCTGCA[CG]GGAG[CG]CAC[CG]C[CG]CT[CG]GCC[CG]GG
GG[CG]TC[CG]TGG[CG]CTGGGAG[CG]AGAGCTT[CG]G[CG
]G[CG]GCTG[CG]GGCAGGG[CG][CG][CG]GAG[CG]GGCTG
CAGGTG[CG]GG[CG]GAGGC[CG]G[CG]GGGGG[CG]C[CG]G
GGG[CG]CTG[CG][CG]GCCCTTGG[CG]G[CG]GGGG[CG][C
G][CG]GGGTGG[CG]GGGGAGGC[CG]AGG[CG]C[CG]GCAGC
TT[CG][CG]C[CG]G[CG]GCTGGAAG[CG]GG[CG]GGCTGCA
[CG]GG[CG]GCT[CG]AGTG[CG]GGGACCCCAGCCCCT[CG]C
CCT[CG]TGAG[CG]C[CG]CCCCTGCCACCTGCTGCCAAGTCAC
[CG]GACTGGGGATG[CG]GGGGG[CG]GGGTG[CG]GGGAGAGG
AAAG[CG]C[CG][CG]GGTGGCA[CG]GTGCCTCTGG[CG]ACA
GAGTAGATGGACC[CG]AAAGAGACC[[CG]]AGGTGACTT[CG]
GGCTGGGCAGGACACCCC[CG]GGAGTTGCCAG[CG]GCTCTCAG
[CG]GG[CG]CCTCTAGGACCCCTTGTCACTG[CG]AGCAG[CG]
CCTGCTCTGGGGAGGGGCT[CG]AAGG[CG]C[CG][CG]CCTTC
TCAA[CG][CG]C[CG]CTG[CG]TCCCCCAGGGGCTCCAAC
SEQ ID NO: 5095
G[CG][CG][CG]GG[CG][CG]GCTCTGGGAG[CG]GGGGAAGG
TGAGGGGAAATGGGACATGATGGCATTAATAAT[CG]TTACCAAA
GA[CG]GTGGGAGTAACAAAGGAATG[CG]TCTC[CG]AGA[CG]
C[CG]CCCAGCCC[CG]GGGTGTTTTCTGG[CG][CG]GT[CG][
CG]GTGCAGGAA[CG][CG]AGCCC[CG]C[CG]C[CG]AGCCTC
C[CG]C[CG]CCTG[CG]TT[CG]TTGG[CG][CG]G[CG]CC[C
G]G[CG]GTAGTGCCACCC[CG]AGGC[CG]AG[CG]CTGCCCC[
CG]C[CG]GCC[CG][CG]GCTGCCAGC[CG]GCCCTGCC[CG][
CG]CC[CG]GGCCC[[CG]][[CG]]AG[CG]GC[CG]CACTTCA
CCTTA[CG]GAGGGGAGATAATGAGATCAATTAGAGG[CG]C[CG
]TCAC[CG][CG]C[CG]GAGACAGCTGC[CG]C[CG]CATAGTA
ATCACC[CG][CG]GGCTGGGTG[CG][CG]GGGGCTCCC[CG]C
TACCTG[CG][CG]CCTGCTGCTCCCACCA[CG][CG]GCAC[CG
]ACC[CG]GG[CG][CG]CCCC[CG]GCCCCTGTC[CG]CAGCCC
ACAGCCACAC[CG][CG]CACCCTACACCCTCCTTG[CG]CCTCT
GCTGGGGAGCTCACCCCCTCCACT[CG]CACAGTG[CG]CTG[CG
]GCC[CG]GGGTGTGGGAGGTCC[CG]GGACTTGGGGT
SEQ ID NO: 5096
GAGAT[CG]GG[CG][CG][CG]GG[CG][CG]GCTCTGGGAG[C
G]GGGGAAGGTGAGGGGAAATGGGACATGATGGCATTAATAAT[C
G]TTACCAAAGA[CG]GTGGGAGTAACAAAGGAATG[CG]TCTC[
CG]AGA[CG]C[CG]CCCAGCCC[CG]GGGTGTTTTCTGG[CG][
CG]GT[CG][CG]GTGCAGGAA[CG][CG]AGCCC[CG][CG]C[
CG]AGCCTCC[CG]C[CG]CCTG[CG]TT[CG]TTGG[CG][CG]
G[CG]CC[CG]G[CG]GTAGTGCCACCC[CG]AGGC[CG]AG[CG
]CTGCCCC[CG][CG]GCC[CG][CG]GCTGCCAGC[CG]GCCCT
GCC[CG][CG]CC[CG]GGCCC[CG][CG]AG[CG]GC[CG]CAC
TTCACCTTA[CG]GAGGGGAGATAATGAGATCAATTAGAGG[CG]
C[CG]TCAC[CG][CG]C[CG]GAGACAGCTGC[CG]C[CG]CAT
AGTAATCACC[CG][CG]GGCTGGGTG[CG][CG]GGGGCTCCC[
CG]CTACCTG[CG][CG]CCTGCTGCTCCCACCA[CG][CG]GCA
C[CG]ACC[CG]GG[CG][CG]CCCC[CG]GCCCCTGTC[CG]CA
GCCCACAGCCACAC[CG][CG]CACCCTACACCCTCCTTG[CG]C
CTCTGCTGGGGAGCTCACCCCCTCCACT[CG]CACAGTG[CG]CT
G[CG]GCC[CG]GGGTGTGGGAGGTCC[CG]GGA
SEQ ID NO: 5097
TGC[CG]GCCT[CG]GAGTC[CG][CG]G[CG]C[CG]G[CG]GC
TAGAGGTCCAGAGG[[CG]]AACCACTTGCTGGTGCAGAAGAGAA
ACCCTCAAATCCCTGGCCTTT[CG][CG]GAGA[CG]CCTGGA[C
G]GGGC[CG]TG[CG]C[CG]TGGACTGAGCAGG[CG]TCT[CG]
GGGAGCACTTCTGCAGAG[CG]AGGACTTCCATGTGAG[CG]ATT
C[[CG]]TTCTCCCCACCACCAATC[CG]ACCTCCCAGC[CG]TC
TC[CG]C[CG]CC[CG]AGCATCCTTGAGGTGGGA[CG]AGCAGG
GGCTTGGATCCCTGC[CG]GC[CG]TCTGGTGTGTGAGGCTTGCA
[[CG]]GCCCCTGGCTGCCC[CG][CG]CCT[CG]C[CG]GAGCC
[CG]AGGGGG[CG]CAGGTC[CG]GGG[CG]AGGGC[[CG]]GC[
[CG]]GGCTGTTTGATGGCTTCACTGAGAAGAGTCAAAGTGCTGT
TGGTGTTGAACTTGAT[CG][CG]GTAGC[CG]GCTT[CG]TGCT
CTTCCTGGCCAAGTGC[CG]GCCCAT[CG][CG]GTG[CG]CAG[
CG]GAGA[CG]CCTTCCA[CG]AGATC[CG]GC[CG][CG][CG]
C[CG]AGGTGGCCAACCTCAG[CG][CG]CACAG[CG]CCAGCCC
CATCCAGGATG[CG]GTCCTGAAG[CG]CCTGT[CG]CTGCTGGA
GGACAT[CG]TGTA
SEQ ID NO: 5098
TTTTGTGAAA[CG]GGCTAGCTGTCAGC[CG]GGATTGATCACTT
GACTGTGTCAGTTGCAGACAGGGCTGGAAGATATGGCTTTAATTT
TCTTTCTTTCTTTCTTTCTTTAACCACAAAACAAAACACAAAACT
GTTTCCTCAAGCCCCTTGTATTGGGGCCTATTGCAGGTGAAGGGG
GCTGACCAAAGGAAGGAGGTGG[CG]CTGATCCAGAGGAGGCTGA
GTGAGGGGTGGAG[CG]GAGCCCAGC[CG]AGGGAGGGAAGGAGA
GGAA[CG]C[CG]GCCCACTAGC[CG]ACCCCAG[CG]CAACCCA
G[CG][[CG]]GTTCCCAGA[CG]CTGC[CG]GGTAACTGATGC[
CG]C[CG]GCACAGCTTCTGTTCTCACTTGTGGAGCTTTGTCTCC
CCT[CG]CTGGGCAGCCCCTGGTAC[CG]CAACC[CG]CCTGGC[
CG]CCTTCC[CG]GCTGAGGCCCC[CG]GGCAGAGCTCCTC[CG]
CAGGGAGCATTCC[CG]TGGACAAACACCCCTTTCCCTGGTAGCT
GTGGCAACAGTTGATCTGTGGCAG[CG]GACCAGGAGGAGGGTCA
AATGGGAGAAGGGA[[CG]]GCCAGCTGGTCCTACCAGAGACTGT
[CG]CTGG[CG]TCCTCAGGGGC[CG]CTG
SEQ ID NO: 5099
GCCC[CG]C[CG]C[CG]AGCCTCC[CG][CG]CCTG[CG]TT[C
G]TTGG[CG][CG]G[CG]CC[CG]G[CG]GTAGTGCCACCC[CG
]AGGC[CG]AG[CG]CTGCCCC[CG]C[CG]GCC[CG][CG]GCT
GCCAGC[CG]GCCCTGCC[CG][CG]CC[CG]GGCCC[CG][CG]
AG[CG]GC[CG]CACTTCACCTTA[CG]GAGGGGAGATAATGAGA
TCAATTAGAGG[CG]C[CG]TCAC[CG][CG]C[CG]GAGACAGC
TGC[CG]C[CG]CATAGTAATCACC[CG][CG]GGCTGGGTG[CG
][CG]GGGGCTCCC[CG]CTACCTG[CG][CG]CCTGCTGCTCCC
ACCA[CG][CG]GCAC[CG]ACC[CG]GG[CG][CG]CCCC[CG]
GCCCCTGTC[CG]CAGCCCACAGCCACAC[CG][CG]CACCCTAC
ACCCTCCTTG[CG]CCTCTGCTGGGGAGCTCACCCCCTCCACT[C
G]CACAGTG[CG]CTG[CG]GCC[CG]GGGTGTGGGAGGTCC[CG
]GGACTTGGGGTGTGAGTGCCTGTGTGGGGGTAGGGGCAGGTGTC
[CG]CTTGTG[CG]CATATGGGCATGAGTGTACATGG[CG]TGTG
CCTGGAGATGGG[CG]AGTGCAGGCTGGAATGTGC[CG]G[CG]T
GGCA[CG]TGTGTGGGCCCAAATAGATG[CG]TGTGTGATCACAT
GTTGTGTT[CG]T
SEQ ID NO: 5100
TTCCCAAACCCCAACCACAGCT[CG]CTC[CG]CCT[CG]AGGAC
CCCTTTTCTGCACCCCCACCTCAG[CG]CCCTCTTCCTGCACCCA
CAAAGAGAGTACTCAGTCATAGGGGTTCAACAGGAGAGAGGAGAC
AGAAGGTACAGG[CG]GTGAGCAGGGACTCAGCCATCATCCCCCT
CAGGTCCCTCCCCACCCAGTTGACAGAG[CG]AATCC[CG]AGTA
ATTGTTTTC[CG]AGGGGGTC[CG]TG[CG][CG]CT[CG]GTGG
[CG]C[CG]CCT[CG]GTCTGGGTTCCCC[CG]AGGAAAAATACC
CACC[CG][CG]AGGGCT[CG]G[[CG]]GCTTTT[CG]ACT[CG
]G[CG]GGGATGAACTGTGGCAACTT[[CG]]GCAGCCCCCAC[C
G][CG]GTG[CG]GAAGTAAAGAGGGCAACATTGG[CG]ACTG[C
G]GCT[CG]GAGGGGCTGGAG[CG][CG]TGAAGC[CG]TGGGGG
[[CG]]C[CG]TG[CG]CCTCC[CG]CTCTCT[CG]TTT[CG]GC
[CG]CAGGTCCTGGGACTC[CG]ACTT[CG]GTGCTC[CG]GGTG
ATAG[CG]GCTG[CG]G[CG]CCTGCAGTCCAGATCCT[CG]CAG
TTCTG[CG]GG[CG]AAGGAGG[[CG]]AA[CG]GAAT[CG]GCC
CCCAGTGGGGAG[[CG]]CAACAAGCCACAGTAGCCAAACC
SEQ ID NO: 5101
GGCTG[CG]G[CG]CCTGCAGTCCAGATCCT[CG]CAGTTCTG[C
G]GG[CG]AAGGAGG[[CG]]AA[[CG]]GAAT[CG]GCCCCCAG
TGGGGAG[[CG]]CAACAAGCCACAGTAGCCAAACCC[CG][CG]
CTCCTGCC[CG]GGCTCCCAGA[CG]AA[CG]CAGC[CG]CAG[C
G]GGGGGC[CG]GGCCC[CG]AGCCCCA[CG]C[CG]C[CG]C[C
G]C[CG][CG]CCAG[CG]GTGGGGGG[CG]GGCTGGGGG[CG]G
GG[CG]GC[CG]GGGCTGCCTTCC[CG]GG[CG]CATATG[CG]A
G[CG]CAGCACC[CG]G[CG]CTGC[CG]AGCCACCTCCCC[CG]
C[CG]CC[CG]CTAGCAAGTTTGG[[CG]]GCTCCAAGCCAGG[C
G][CG]CCTCAGGATCCAGGCTCATTTGCTTCCACCTAGCTT[CG
]GTGCCCCCTGCTAGG[CG]GGGACCCT[CG]AGAG[CG]ATGC[
CG]ATGGATTTGATTTTAGTTGTGTGGTTCTGTGTGTGCACTGCC
AGGACAGGTAAGCATGACTGTGG[[CG]]GTTAGAGGGATC[CG]
GGAAATGGGGGTGCCCACAGACCA[CG]G[CG]G[CG]TGGGGAG
ACCTGGAGC[CG]AGCTTTG[CG]CTGGTCTGGGGAA[CG]G[CG
]GTAG[CG]TGGAC[CG]GTTCCTGGGATCTCTTTGCCCTGCT[C
G]GAGTGA
SEQ ID NO: 5102
TGGAGAT[CG]GG[CG][CG][CG]GG[CG][CG]GCTCTGGGAG
[CG]GGGGAAGGTGAGGGGAAATGGGACATGATGGCATTAATAAT
[CG]TTACCAAAGA[CG]GTGGGAGTAACAAAGGAATG[CG]TCT
C[CG]AGA[CG]C[CG]CCCAGCCC[CG]GGGTGTTTTCTGG[CG
][CG]GT[CG][CG]GTGCAGGAA[CG][CG]AGCCC[CG]C[CG
]C[CG]AGCCTCC[CG]C[CG]CCTG[CG]TT[CG]TTGG[CG][
CG]G[CG]CC[CG]G[CG]GTAGTGCCACCC[CG]AGGC[CG]AG
[CG]CTGCCCC[CG]C[CG]GCC[CG][CG]GCTGCCAGC[CG]G
CCCTGCC[CG][CG]CC[CG]GGCCC[CG][CG]AG[CG]GC[CG
]CACTTCACCTTA[CG]GAGGGGAGATAATGAGATCAATTAGAGG
[CG]C[CG]TCAC[CG][CG][CG]GAGACAGCTGC[CG]C[CG]
CATAGTAATCACC[CG][CG]GGCTGGGTG[CG][CG]GGGGCTC
CC[CG]CTACCTG[CG][CG]CCTGCTGCTCCCACCA[CG][CG]
GCAC[CG]ACC[CG]GG[CG][[CG]]CCCC[CG]GCCCCTGTC[
CG]CAGCCCACAGCCACAC[CG][CG]CACCCTACACCCTCCTTG
[CG]CCTCTGCTGGGGAGCTCACCCCCTCCACT[CG]CACAGTG[
CG]CTG[CG]GCC[CG]GGGTGTGGGAGGTCC[CG]G
SEQ ID NO: 5103
[CG]GTGCTC[CG]GGTGATAG[CG]GCTG[CG]G[CG]CCTGCA
GTCCAGATCCT[CG]CAGTTCTG[CG]GG[CG]AAGGAGG[[CG]
]AA[[CG]]GAAT[CG]GCCCCCAGTGGGGAG[CG]CAACAAGCC
ACAGTAGCCAAACCC[CG][CG]CTCCTGCC[CG]GGCTCCCAGA
[CG]AA[CG]CAGC[CG]CAG[CG]GGGGGC[CG]GGCCC[CG]A
GCCCCA[CG]C[CG]C[CG]C[CG]C[CG][CG]CCAG[CG]GTG
GGGGG[CG]GGCTGGGGG[CG]GGG[CG]GC[CG]GGGCTGCCTT
CC[CG]GG[CG]CATATG[CG]AG[[CG]]CAGCACC[CG]G[CG
]CTGC[CG]AGCCACCTCCCC[CG]C[CG]CC[CG]CTAGCAAGT
TTGG[CG]GCTCCAAGCCAGG[CG][CG]CCTCAGGATCCAGGCT
CATTTGCTTCCACCTAGCTT[CG]GTGCCCCCTGCTAGG[CG]GG
GACCCT[CG]AGAG[CG]ATGC[CG]ATGGATTTGATTTTAGTTG
TGTGGTTCTGTGTGTGCACTGCCAGGACAGGTAAGCATGACTGTG
G[[CG]]GTTAGAGGGATC[CG]GGAAATGGGGGTGCCCACAGAC
CA[CG]G[CG]G[CG]TGGGGAGACCTGGAGC[CG]AGCTTTG[C
G]CTGGTCTGGGGAA[[CG]]G[CG]GTAG[CG]TGGAC[CG]GT
TCCTGGGATCTC
SEQ ID NO: 5104
GGGCTAGCTGTCAGC[CG]GGATTGATCACTTGACTGTGTCAGTT
GCAGACAGGGCTGGAAGATATGGCTTTAATTTTCTTTCTTTCTTT
CTTTCTTTAACCACAAAACAAAACACAAAACTGTTTCCTCAAGCC
CCTTGTATTGGGGCCTATTGCAGGTGAAGGGGGCTGACCAAAGGA
AGGAGGTGG[CG]CTGATCCAGAGGAGGCTGAGTGAGGGGTGGAG
[CG]GAGCCCAGC[CG]AGGGAGGGAAGGAGAGGAA[CG]C[CG]
GCCCACTAGC[CG]ACCCCAG[CG]CAACCCAG[CG][[CG]]GT
TCCCAGA[[CG]]CTGC[CG]GGTAACTGATGC[CG]C[CG]GCA
CAGCTTCTGTTCTCACTTGTGGAGCTTTGTCTCCCCT[CG]CTGG
GCAGCCCCTGGTAC[[CG]]CAACC[CG]CCTGGC[CG]CCTTCC
[CG]GCTGAGGCCCC[CG]GGCAGAGCTCCTC[CG]CAGGGAGCA
TTCC[CG]TGGACAAACACCCCTTTCCCTGGTAGCTGTGGCAACA
GTTGATCTGTGGCAG[CG]GACCAGGAGGAGGGTCAAATGGGAGA
AGGGA[[CG]]GCCAGCTGGTCCTACCAGAGACTGT[CG]CTGG[
CG]TCCTCAGGGGC[CG]CTGCAGGGCCACCT
SEQ ID NO: 5105
TTTCTTTCTTTAACCACAAAACAAAACACAAAACTGTTTCCTCAA
GCCCCTTGTATTGGGGCCTATTGCAGGTGAAGGGGGCTGACCAAA
GGAAGGAGGTGG[CG]CTGATCCAGAGGAGGCTGAGTGAGGGGTG
GAG[CG]GAGCCCAGC[CG]AGGGAGGGAAGGAGAGGAA[CG]C[
CG]GCCCACTAGC[CG]ACCCCAG[CG]CAACCCAG[CG][CG]G
TTCCCAGA[[CG]]CTGC[CG]GGTAACTGATGC[CG]C[CG]GC
ACAGCTTCTGTTCTCACTTGTGGAGCTTTGTCTCCCCT[CG]CTG
GGCAGCCCCTGGTAC[CG]CAACC[CG]CCTGGC[CG]CCTTCC[
CG]GCTGAGGCCCC[CG]GGCAGAGCTCCTC[CG]CAGGGAGCAT
TCC[CG]TGGACAAACACCCCTTTCCCTGGTAGCTGTGGCAACAG
TTGATCTGTGGCAG[CG]GACCAGGAGGAGGGTCAAATGGGAGAA
GGGA[[CG]]GCCAGCTGGTCCTACCAGAGACTGT[CG]CTGG[C
G]TCCTCAGGGGC[CG]CTGCAGGGCCACCTTGTGTGCTTGCCCT
TTCCCCA[CG]GTAGACTGCCC[CG]GGAGGGGGACTGTGCTGCC
TGAATTCTTGTC[CG]GGTGGGACTGGCCT
SEQ ID NO: 5106
ACAGAAGGTACAGG[CG]GTGAGCAGGGACTCAGCCATCATCCCC
CTCAGGTCCCTCCCCACCCAGTTGACAGAG[CG]AATCC[CG]AG
TAATTGTTTTC[CG]AGGGGGTC[CG]TG[CG][CG]CT[CG]GT
GG[CG]C[CG]CCT[CG]GTCTGGGTTCCCC[CG]AGGAAAAATA
CCCACC[CG][CG]AGGGCT[CG]G[CG]GCTTTT[CG]ACT[CG
]G[CG]GGGATGAACTGTGGCAACTT[[CG]]GCAGCCCCCAC[C
G][CG]GTG[CG]GAAGTAAAGAGGGCAACATTGG[CG]ACTG[C
G]GCT[CG]GAGGGGCTGGAG[CG][CG]TGAAGC[CG]TGGGGG
[[CG]]C[CG]TG[CG]CCTCC[CG]CTCTCT[CG]TTT[CG]GC
[CG]CAGGTCCTGGGACTC[CG]ACTT[CG]GTGCTC[CG]GGTG
ATAG[CG]GCTG[CG]G[CG]CCTGCAGTCCAGATCCT[CG]CAG
TTCTG[CG]GG[CG]AAGGAGG[[CG]]AA[CG]GAAT[CG]GCC
CCCAGTGGGGAG[CG]CAACAAGCCACAGTAGCCAAACCC[CG][
CG]CTCCTGCC[CG]GGCTCCCAGA[CG]AA[CG]CAGC[CG]CA
G[CG]GGGGGC[CG]GGCCC[CG]AGCCCCA[CG]C[CG]C[CG]
C[CG]C[CG][CG]CCAG[CG]GTGGGGGG[CG]GGCTGGGGG[C
G]GGG[CG]GC[CG]GGGCTGCCT
SEQ ID NO: 5107
CCCCTTTTCTGCACCCCCACCTCAG[CG]CCCTCTTCCTGCACCC
ACAAAGAGAGTACTCAGTCATAGGGGTTCAACAGGAGAGAGGAGA
CAGAAGGTACAGG[CG]GTGAGCAGGGACTCAGCCATCATCCCCC
TCAGGTCCCTCCCCACCCAGTTGACAGAG[CG]AATCC[CG]AGT
AATTGTTTTC[CG]AGGGGGTC[CG]TG[CG][CG]CT[CG]GTG
G[CG]C[CG]CCT[CG]GTCTGGGTTCCCC[CG]AGGAAAAATAC
CCACC[CG][CG]AGGGCT[CG]G[CG]GCTTTT[CG]ACT[CG]
G[CG]GGGATGAACTGTGGCAACTT[[CG]]GCAGCCCCCAC[CG
][CG]GTG[CG]GAAGTAAAGAGGGCAACATTGG[CG]ACTG[CG
]GCT[CG]GAGGGGCTGGAG[CG][CG]TGAAGC[CG]TGGGGG[
[CG]]C[CG]TG[CG]CCTCC[CG]CTCTCT[CG]TTT[CG]GC[
CG]CAGGTCCTGGGACTC[CG]ACTT[CG]GTGCTC[CG]GGTGA
TAG[CG]GCTG[CG]G[CG]CCTGCAGTCCAGATCCT[CG]CAGT
TCTG[CG]GG[CG]AAGGAGG[[CG]]AA[[CG]]GAAT[CG]GC
CCCCAGTGGGGAG[[CG]]CAACAAGCCACAGTAGCCAAACCC[C
G][CG]CTCCTGCC[CG]GGCTCCCAGA[CG]AA[CG]CAGC[CG
]C
SEQ ID NO: 5108
[CG]GGGCT[CG]GGGCTTTGGTACAGAGGAGCAGCAGAAGC[CG
]C[CG]C[CG]CAGA[CG]CC[CG]GATG[CG]GAGGACCCAGAG
[CG]CTG[CG]CAGCTGAGGAGCACCTGGGGAGCCAC[CG]C[CG
]G[CG]CCAACTTTTGCCATCC[CG]GGAGCCATAG[CG]GCAGA
GAGGGGGTCC[CG]GACC[CG]AGGTGGCAG[[CG]]AGGGGCTC
TGCTTTGGCAGCTGT[CG]GGGCTGGAGCTCAGGACCCCAACTCC
AGCCAAGT[CG]GGC[CG][CG]G[CG]GGGCAGG[CG]TAGGCT
G[CG]CAGGCAGGGGTGGC[CG][CG]GGTGCTGAGCCAGT[CG]
C[CG]C[CG][CG]CAGG[CG]GG[CG]CTGGAGTCTCAGCTACC
TGG[CG][CG]AC[CG]CCAGCAGCACCCAG[CG][CG]GGGC[C
G]GGAGCTGCTGGGGGCCCAGGCTC[[CG]]CTCTCCCCAC[CG]
CTCTGCAC[CG]CTGC[CG]GCTG[CG]GACAGACC[CG]ATG[C
G]CCACCACCAC[CG]CAGTTGCCA[CG]ACCCCCATGGAAT[CG
]CTGG[CG]CCTC[CG]CTC[CG]GTTCTCTAACC[CG]G[CG]C
ACTCTGGGATCCCAAAGCTTGC[CG]TGGGTTGGAGGCTTCCTAC
TCTTTGTG[[CG]]TTGAGGCTTTGACCTGACCCAGAG[CG]CAG
SEQ ID NO: 5109
ATCTCTATCTCCATCATCTCCATCTCCATCATCTCCATCATCTCC
ATCTCCATCTCCTACTCCATCATCTCCATCTCCATCTTCTCCATC
ATCTCCATCTTCTCCATCATCTCCATCTTCTCCATCATCTCCATC
TTCATCATCTCCATTTCCATCATCTCCATCTCCATCATCATCTCT
ATCTCCATCATCTCCATCTCCATCATCTCCATCATCTCCATCTCC
ATCTCCATCTCCATCATCTAC[CG]TCTCCAATCTCCATCTC[CG
]AAGTTATGCCCACTTCCT[CG]AAGTTTGGAGCCA[[CG]][CG
]AACTACACTGCCCAGAAGG[CG]C[CG][CG]C[CG]TGAGC[C
G][CG]ATGCTTGGCCAATGAAAAGAGGTCTACC[CG]AGAGTG[
CG]A[CG][CG]CAATGGG[CG]GGACTTC[CG]G[CG]TCTCCC
CT[CG]G[CG]GTTGCTTT[CG]CTGCCCTCAAGAGAACTCAGCT
TGC[CG]GAAGCTGGTTGTT[CG]CTG[CG]G[CG]ACCAGCTC[
CG]GAAAG[CG][CG]GTGGGGA[[CG]][CG]CTGTGTTCT[CG
]CAGCTCAGAGG[CG]GGTCTGAGGCT[CG]GTGG[CG]G[CG]C
CCAGGGTGGCC[CG]GGCCCTTTCCT[CG]GT[CG]TTGTCTCAC
[CG]CCA
SEQ ID NO: 5110
TCTCCATCTTCATCATCTCCATTTCCATCATCTCCATCTCCATCA
TCATCTCTATCTCCATCATCTCCATCTCCATCATCTCCATCATCT
CCATCTCCATCTCCATCTCCATCATCTAC[CG]TCTCCAATCTCC
ATCTC[CG]AAGTTATGCCCACTTCCT[CG]AAGTTTGGAGCCA[
CG][CG]AACTACACTGCCCAGAAGG[CG]C[CG][CG]C[CG]T
GAGC[CG][CG]ATGCTTGGCCAATGAAAAGAGGTCTACC[CG]A
GAGTG[CG]A[CG][CG]CAATGGG[CG]GGACTTC[CG]G[CG]
TCTCCCCT[CG]G[CG]GTTGCTTT[[CG]]CTGCCCTCAAGAGA
ACTCAGCTTGC[CG]GAAGCTGGTTGTT[[CG]]CTG[CG]G[CG
]ACCAGCTC[CG]GAAAG[CG][CG]GTGGGGA[[CG]][CG]CT
GTGTTCT[CG]CAGCTCAGAGG[CG]GGTCTGAGGCT[CG]GTGG
[CG]G[CG]CCCAGGGTGGCC[CG]GGCCCTTTCCT[CG]GT[CG
]TTGTCTCAC[CG]CCACAGGCTC[CG]ATGG[CG]G[CG]GCCA
[CG]CTGAGGGACCC[CG]CTCAGGTGAG[CG]C[CG][CG]TCC
TCC[CG]GCCTCCCC[CG]AATCCTAAAGCCCTGTGAGGGC[CG]
CCTGCTCAGGTCCC[CG]GGTGCAGAACTGTGG
SEQ ID NO: 5111
AAGAGAAACCCTCAAATCCCTGGCCTTT[CG][CG]GAGA[CG]C
CTGGA[CG]GGGC[CG]TG[CG]C[CG]TGGACTGAGCAGG[CG]
TCT[CG]GGGAGCACTTCTGCAGAG[CG]AGGACTTCCATGTGAG
[CG]ATTC[[CG]]TTCTCCCCACCACCAATC[CG]ACCTCCCAG
C[CG]TCTC[CG]C[CG]CC[CG]AGCATCCTTGAGGTGGGA[CG
]AGCAGGGGCTTGGATCCCTGC[CG]GC[CG]TCTGGTGTGTGAG
GCTTGCA[[CG]]GCCCCTGGCTGCCC[CG][CG]CCT[CG][CG
]GAGCC[CG]AGGGGG[CG]CAGGTC[CG]GGG[CG]AGGGC[[C
G]]GC[CG]GGCTGTTTGATGGCTTCACTGAGAAGAGTCAAAGTG
CTGTTGGTGTTGAACTTGAT[CG][CG]GTAGC[CG]GCTT[CG]
TGCTCTTCCTGGCCAAGTGC[CG]GCCCAT[CG][CG]GTG[CG]
CAG[CG]GAGA[CG]CCTTCCA[CG]AGATC[CG]GC[CG][CG]
[CG]C[CG]AGGTGGCCAACCTCAG[CG][CG]CACAG[CG]CCA
GCCCCATCCAGGATG[CG]GTCCTGAAG[CG]CCTGT[CG]CTGC
TGGAGGACAT[CG]TGTAC[CG]GCAGCTGAATGGTAAGGA[CG]
CA[CG]C[CG]G[CG]CCTC[CG]GGGCT[CG]A[CG][CG]GG[
CG]GGC[CG]GGCAC
SEQ ID NO: 5112
TCATCATCTCTATCTCCATCATCTCCATCTCCATCATCTCCATCA
TCTCCATCTCCATCTCCATCTCCATCATCTAC[CG]TCTCCAATC
TCCATCTC[CG]AAGTTATGCCCACTTCCT[CG]AAGTTTGGAGC
CA[[CG]][CG]AACTACACTGCCCAGAAGG[CG]C[CG][CG]C
[CG]TGAGC[CG][CG]ATGCTTGGCCAATGAAAAGAGGTCTACC
[CG]AGAGTG[CG]A[CG][CG]CAATGGG[CG]GGACTTC[CG]
G[CG]TCTCCCCT[CG]G[CG]GTTGCTTT[CG]CTGCCCTCAAG
AGAACTCAGCTTGC[CG]GAAGCTGGTTGTT[[CG]]CTG[CG]G
[CG]ACCAGCTC[CG]GAAAG[CG][CG]GTGGGGA[[CG]][CG
]CTGTGTTCT[CG]CAGCTCAGAGG[CG]GGTCTGAGGCT[CG]G
TGG[CG]G[CG]CCCAGGGTGGCC[CG]GGCCCTTTCCT[CG]GT
[CG]TTGTCTCAC[CG]CCACAGGCTC[CG]ATGG[CG]G[CG]G
CCA[CG]CTGAGGGACCC[CG]CTCAGGTGAG[CG]C[CG][CG]
TCCTCC[CG]GCCTCCCC[CG]AATCCTAAAGCCCTGTGAGGGC[
CG]CCTGCTCAGGTCCC[CG]GGTGCAGAACTGTGGGG[CG]TT[
CG]TGGG[CG]G[CG]TCC[CG]TTTCTGACA[CG]CAGTGTGAT
SEQ ID NO: 5113
GCAGAAGAGAAACCCTCAAATCCCTGGCCTTT[CG][CG]GAGA[
CG]CCTGGA[CG]GGGC[CG]TG[CG]C[CG]TGGACTGAGCAGG
[CG]TCT[CG]GGGAGCACTTCTGCAGAG[CG]AGGACTTCCATG
TGAG[CG]ATTC[[CG]]TTCTCCCCACCACCAATC[CG]ACCTC
CCAGC[CG]TCTC[CG]C[CG]CC[CG]AGCATCCTTGAGGTGGG
A[CG]AGCAGGGGCTTGGATCCCTGC[CG]GC[CG]TCTGGTGTG
TGAGGCTTGCA[CG]GCCCCTGGCTGCCC[CG][CG]CCT[CG]C
[CG]GAGCC[CG]AGGGGG[CG]CAGGTC[CG]GGG[CG]AGGGC
[CG]GC[CG]GGCTGTTTGATGGCTTCACTGAGAAGAGTCAAAGT
GCTGTTGGTGTTGAACTTGAT[CG][CG]GTAGC[CG]GCTT[CG
]TGCTCTTCCTGGCCAAGTGC[CG]GCCCAT[CG][CG]GTG[CG
]CAG[CG]GAGA[CG]CCTTCCA[CG]AGATC[CG]GC[CG][CG
][CG]C[CG]AGGTGGCCAACCTCAG[CG][CG]CACAG[CG]CC
AGCCCCATCCAGGATG[CG]GTCCTGAAG[CG]CCTGT[CG]CTG
CTGGAGGACAT[CG]TGTAC[CG]GCAGCTGAATGGTAAGGA[CG
]CA[CG]C[CG]G[CG]CCTC[CG]GGGCT[CG]A[CG][CG]GG
[CG]GGC[CG]G
SEQ ID NO: 5114
TCTCCATCATCTCCATCTCCATCTCCATCTCCATCATCTAC[CG]
TCTCCAATCTCCATCTC[CG]AAGTTATGCCCACTTCCT[CG]AA
GTTTGGAGCCA[[CG]][CG]AACTACACTGCCCAGAAGG[CG]C
[CG][CG]C[CG]TGAGC[CG][CG]ATGCTTGGCCAATGAAAAG
AGGTCTACC[CG]AGAGTG[CG]A[CG][CG]CAATGGG[CG]GG
ACTTC[CG]G[CG]TCTCCCCT[CG]G[CG]GTTGCTTT[CG]CT
GCCCTCAAGAGAACTCAGCTTGC[CG]GAAGCTGGTTGTT[[CG]
]CTG[CG]G[CG]ACCAGCTC[CG]GAAAG[CG][CG]GTGGGGA
[[CG]][CG]CTGTGTTCT[CG]CAGCTCAGAGG[CG]GGTCTGA
GGCT[CG]GTGG[CG]G[CG]CCCAGGGTGGCC[CG]GGCCCTTT
CCT[CG]GT[CG]TTGTCTCAC[CG]CCACAGGCTC[CG]ATGG[
CG]G[CG]GCCA[CG]CTGAGGGACCC[CG]CTCAGGTGAG[CG]
C[CG][CG]TCCTCC[CG]GCCTCCCC[CG]AATCCTAAAGCCCT
GTGAGGGC[CG]CCTGCTCAGGTCCC[CG]GGTGCAGAACTGTGG
GG[CG]TT[CG]TGGG[CG]G[CG]TCC[CG]TTTCTGACA[CG]
CAGTGTGATGGGGTGGCAATGGAGGGCAAGGGGCC[CG]GCT[CG
]CAA
SEQ ID NO: 5115
TCT[CG]TTT[CG]GC[CG]CAGGTCCTGGGACTC[CG]ACTT[C
G]GTGCTC[CG]GGTGATAG[CG]GCTG[CG]G[CG]CCTGCAGT
CCAGATCCT[CG]CAGTTCTG[CG]GG[CG]AAGGAGG[[CG]]A
A[CG]GAAT[[CG]]GCCCCCAGTGGGGAG[CG]CAACAAGCCAC
AGTAGCCAAACCC[CG][CG]CTCCTGCC[CG]GGCTCCCAGA[C
G]AA[CG]CAGC[CG]CAG[CG]GGGGGC[CG]GGCCC[CG]AGC
CCCA[CG]C[CG]C[CG]C[CG]C[CG][CG]CCAG[CG]GTGGG
GGG[CG]GGCTGGGGG[CG]GGG[CG]GC[CG]GGGCTGCCTTCC
[CG]GG[CG]CATATG[CG]AG[CG]CAGCACC[CG]G[CG]CTG
C[CG]AGCCACCTCCCC[CG]C[CG]CC[CG]CTAGCAAGTTTGG
[[CG]]GCTCCAAGCCAGG[CG][CG]CCTCAGGATCCAGGCTCA
TTTGCTTCCACCTAGCTT[CG]GTGCCCCCTGCTAGG[CG]GGGA
CCCT[CG]AGAG[CG]ATGC[CG]ATGGATTTGATTTTAGTTGTG
TGGTTCTGTGTGTGCACTGCCAGGACAGGTAAGCATGACTGTGG[
[CG]]GTTAGAGGGATC[CG]GGAAATGGGGGTGCCCACAGACCA
[CG]G[CG]G[CG]TGGGGAGACCTGGAGC[CG]AGCTTTG[CG]
CTGGTCTGGG
SEQ ID NO: 5116
G[CG]CTGCTTTTC[CG][CG]CTCAGTGATCCAGTTAGGAGCTC
TCCC[CG]CAGCAGCTG[CG][CG]GGACTTTGCATTCCAGATGT
CCCCAGCCCTCCAGGCTAAAGCAG[CG]GCCACCTGAAGTTGAGG
AATGGGG[CG]TGA[CG]ACCACCAGGTCC[CG]CAGTGACCA[C
G]GGAGGGTGGGGAGAAGGAGTCCAGCTC[CG]CCTTGAG[CG]T
[CG]GGTCCCTGGGCAGGACCCTC[CG]GGCCA[CG]GGC[CG]C
TGTC[CG]TTGGGCACC[CG]GGCAGGAGTCTG[CG]AA[CG]CT
GAGTTTCTCTGGAAAGG[CG][CG]GACCCC[[CG]]GAGGAGC[
CG]TCCCAGC[CG]CCTGGGGAGA[CG]CCCCCTCCTCTCC[CG]
GTCTTCTTTCC[CG]GGCACACCCTCCCTAGCC[CG]GG[CG]GG
GGGGTTCTCCATCTGC[CG]CCCCTCC[CG]G[CG]GGCAGG[CG
]AACTCTCTCCTAC[CG]CACCCTCAATCTGTC[CG][CG]GT[C
G][CG]GGGCT[CG]G[CG]GGGG[CG]GGGCC[CG]GG[CG]GG
GG[CG]GGGG[CG]GGGC[CG]GGGG[CG]GGG[CG]GGC[CG]G
[CG]GCTG[CG]GAGGCTG[CG]GAGGCTG[CG]GGAGAAAAGTT
GTCC[CG]GC[CG]GAG[CG][CG]AGCAG[CG]G[CG]GAGC[C
G]GAGC
SEQ ID NO: 5117
AGGGGCACAGCCTGGAGCTTGCTACTCTCTAGGATCTGTTGTCAA
AGCAGTTTGCAAAAACAGTCAGGCAAAACCTTGAGG[CG]TA[CG
]TA[CG]TTATCACCCA[CG]GATAT[CG][CG]AAAGACACAGC
TTAGCCCAGAGCCTATGCTAAGAACCCTCATCCACCATCTGAGGG
GCAGGAGG[CG][CG]CAAGGTGTC[CG]AAGGGGCATGGGAATT
CCTATAAGAAA[CG]GTCCCTTCTGGATAGT[CG]GAACATAGAG
GAAAA[CG]GGGGTGGGTTAGGGGAACACTTACTCTTGTGGGGCC
CTG[CG]CTGG[[CG]]AATGTCAAGCCTCCAGGTTCACAAATGG
CTGGAGGTGCT[CG]GGGTTAGCTCC[CG]AGGG[CG]GCAAG[C
G]C[CG]AC[CG]GTCCT[CG]CT[CG]CTAGGCAGG[CG]CCTC
[CG]GATTC[CG]TTCATGCCTCT[CG]GAGCTACAGGGCTTGGT
G[CG]GAGA[CG]CAGGG[CG]GG[[CG]][CG]CTGGGCTCTGG
GTGTC[[CG]]CAACCCAAAGTGCAGCTGGTGTCTTGA[CG]CTA
C[[CG]]TCTATGCACAAGC[CG]G[CG]G[CG]G[CG]G[[CG]
]GAGCTTGGAGGAGGCAGGGAGATTCCAAATAAATCCACTAATTC
TGGGCTGAG[CG]CT
SEQ ID NO: 5118
ACTAATCAATTTGAGATGACTTCCCCCTCATTCCCAAAGCTAGAG
GAGGAAGGGGGCTGAAAGGGGCTCAGAGCAGTGGAAGGTCCCAGG
CCCAGCTGGGGTTGGGA[[CG]]TGTGTGTGTGTGTGTGTGTGTG
TGTGTGTGTGTGTGTGTGTGTAGAGGGGTGGGTTC[CG]GGGGGG
TGGTGGAAGAGGGC[CG]AGGGGGGAGGATAGAAGGAGGGGGTAG
AGTTTCAGGG[CG]GGGAGGGGGG[CG]CTGGGG[CG]CAGTGA[
CG]GGAACCAATGAGCTGCCAACT[[CG]][[CG]][CG]TCTC[
CG]G[[CG]]TGACTGC[[CG]]AGATTGA[CG]TGGAGGACA[C
G]TCAAATTGATTCC[[CG]]CA[CG]CTGCAGCCTCC[CG]GTC
AGA[CG]AATTTCTCCCAAT[CG]GATGAAGTTCACCCTGGGCCT
GGGGT[[CG]][CG]GG[CG]TGGAGAGTGTCCTGGGAGGGGGCA
GCAG[CG]G[CG]G[CG]GCAGGCCCTGGAG[CG]GG[CG]GCAG
[CG][CG]CTC[CG]CTGC[CG][CG]CACAG[CG][CG]TCTCC
AGCC[CG][CG]GCTGGGC[CG]C[CG][CG]GCTCT[CG]GCTC
T[CG]GG[CG]CCCTCCCTCTATGCCTCTCA[CG][CG]G[CG]G
[CG]G[CG]G[CG]CCCAAGCTCTCC[CG]GACTG[CG]CC
SEQ ID NO: 5119
TTCCCCCTCATTCCCAAAGCTAGAGGAGGAAGGGGGCTGAAAGGG
GCTCAGAGCAGTGGAAGGTCCCAGGCCCAGCTGGGGTTGGGA[[C
G]]TGTGTGTGTGTGTGTGTGTGTGTGTGTGTGTGTGTGTGTGTG
TAGAGGGGTGGGTTC[CG]GGGGGGTGGTGGAAGAGGGC[CG]AG
GGGGGAGGATAGAAGGAGGGGGTAGAGTTTCAGGG[CG]GGGAGG
GGGG[CG]CTGGGG[CG]CAGTGA[CG]GGAACCAATGAGCTGCC
AACT[[CG]][[CG]][CG]TCTC[CG]G[CG]TGACTGC[CG]A
GATTGA[[CG]]TGGAGGACA[CG]TCAAATTGATTCC[[CG]]C
A[CG]CTGCAGCCTCC[CG]GTCAGA[CG]AATTTCTCCCAAT[C
G]GATGAAGTTCACCCTGGGCCTGGGGT[[CG]][CG]GG[CG]T
GGAGAGTGTCCTGGGAGGGGGCAGCAG[CG]G[CG]G[CG]GCAG
GCCCTGGAG[CG]GG[CG]GCAG[CG][CG]CTC[CG]CTGC[CG
][CG]CACAG[CG][CG]TCTCCAGCC[CG][CG]GCTGGGC[CG
]C[CG][CG]GCTCT[CG]GCTCT[CG]GG[CG]CCCTCCCTCTA
TGCCTCTCA[CG][CG]G[CG]G[CG]G[CG]G[CG]CCCAAGCT
CTCC[CG]GACTG[CG]C[CG]GGCCCAGCCC[CG]GCCACCC
SEQ ID NO: 5120
TTTGAGATGACTTCCCCCTCATTCCCAAAGCTAGAGGAGGAAGGG
GGCTGAAAGGGGCTCAGAGCAGTGGAAGGTCCCAGGCCCAGCTGG
GGTTGGGA[[CG]]TGTGTGTGTGTGTGTGTGTGTGTGTGTGTGT
GTGTGTGTGTGTAGAGGGGTGGGTTC[CG]GGGGGGTGGTGGAAG
AGGGC[CG]AGGGGGGAGGATAGAAGGAGGGGGTAGAGTTTCAGG
G[CG]GGGAGGGGGG[CG]CTGGGG[CG]CAGTGA[CG]GGAACC
AATGAGCTGCCAACT[[CG]][[CG]][CG]TCTC[CG]G[[CG]
]TGACTGC[[CG]]AGATTGA[CG]TGGAGGACA[CG]TCAAATT
GATTCC[CG]CA[CG]CTGCAGCCTCC[CG]GTCAGA[CG]AATT
TCTCCCAAT[CG]GATGAAGTTCACCCTGGGCCTGGGGT[[CG]]
[CG]GG[CG]TGGAGAGTGTCCTGGGAGGGGGCAGCAG[CG]G[C
G]G[CG]GCAGGCCCTGGAG[CG]GG[CG]GCAG[CG][CG]CTC
[CG]CTGC[CG][CG]CACAG[CG][CG]TCTCCAGCC[CG][CG
]GCTGGGC[CG]C[CG][CG]GCTCT[CG]GCTCT[CG]GG[CG]
CCCTCCCTCTATGCCTCTCA[CG][CG]G[CG]G[CG]G[CG]G[
[CG]]CCCAAGCTCTCC[CG]GACTG[CG]C[CG]GGCCCAGC
SEQ ID NO: 5121
TACTAACACACTAATCAATTTGAGATGACTTCCCCCTCATTCCCA
AAGCTAGAGGAGGAAGGGGGCTGAAAGGGGCTCAGAGCAGTGGAA
GGTCCCAGGCCCAGCTGGGGTTGGGA[[CG]]TGTGTGTGTGTGT
GTGTGTGTGTGTGTGTGTGTGTGTGTGTGTAGAGGGGTGGGTTC[
CG]GGGGGGTGGTGGAAGAGGGC[CG]AGGGGGGAGGATAGAAGG
AGGGGGTAGAGTTTCAGGG[CG]GGGAGGGGGG[CG]CTGGGG[C
G]CAGTGA[CG]GGAACCAATGAGCTGCCAACT[[CG]][[CG]]
[CG]TCTC[CG]G[[CG]]TGACTGC[[CG]]AGATTGA[[CG]]
TGGAGGACA[[CG]]TCAAATTGATTCC[[CG]]CA[CG]CTGCA
GCCTCC[CG]GTCAGA[CG]AATTTCTCCCAAT[CG]GATGAAGT
TCACCCTGGGCCTGGGGT[CG][CG]GG[CG]TGGAGAGTGTCCT
GGGAGGGGGCAGCAG[CG]G[CG]G[CG]GCAGGCCCTGGAG[CG
]GG[CG]GCAG[CG][CG]CTC[CG]CTGC[CG][CG]CACAG[C
G][CG]TCTCCAGCC[CG][CG]GCTGGGC[CG]C[CG][CG]GC
TCT[CG]GCTCT[CG]GG[CG]CCCTCCCTCTATGCCTCTCA[CG
][CG]G[CG]G[CG]G[CG]G[[CG]]CCCAAGCTCTCC
SEQ ID NO: 5122
G[CG]AGCCTTTACCTGCCAGGCTGCC[CG]GCCTCTGGAGGGTG
GC[CG]TGGCATACAGCTCCTG[CG]AGTGCTTGCTGTACTGCT[
CG]GA[CG][CG]TGGACCAGG[CG]CTTGGTGGG[CG]ACAGGG
TGG[[CG]]TA[CG]TGC[CG]ATGGTGGAGCTCAGCTGGTGGAT
GGG[CG]AGGAGGAGATGGTGGACTGCA[CG]GTGGGGGG[CG]A
GGTCA[CG][CG]GAT[CG]GGGACAGGC[CG]GC[CG]AGGACA
[CG]A[CG]ATGTTGATGGG[CG]AGCTGGTGCTGTAGGACTTGG
CCAGG[CG]GCTGGG[CG]ACTGCTTGGG[CG]AGGAGC[CG][[
CG]][CG]G[CG][CG]G[CG]TAGGTGG[CG]CCCT[CG]GGGG
C[CG]AGC[CG]C[CG][CG]CTGCAGCTTGGTGGG[CG]AAC[C
G]CCCTGGGG[CG][CG]GCCAG[CG]GGGAGCCCC[CG][CG][
CG]G[CG]G[CG][CG]GGCAG[CG]TGGAGCTGGAGTAGTAGAG
[CG][CG]G[CG]G[CG]G[CG]G[CG]G[CG]GG[CG]G[CG][
CG]T[CG]GGCAGGTGGAAGG[CG]CTGCCCAGGCTGGG[CG][C
G]AA[CG]GCTCC[CG][CG]G[CG]G[CG]G[CG]G[CG]G[CG
]G[CG]G[CG][CG]GGCT[CG]GGCCC[CG]CCAGGTGGC[CG]
G[CG][CG]GCTGGT[CG]TGCCCTGTGCC[CG]GGAGAGGAGAG
AAC
SEQ ID NO: 5123
AAA[CG]AGTACTAGTACTAACACACTAATCAATTTGAGATGACT
TCCCCCTCATTCCCAAAGCTAGAGGAGGAAGGGGGCTGAAAGGGG
CTCAGAGCAGTGGAAGGTCCCAGGCCCAGCTGGGGTTGGGA[[CG
]]TGTGTGTGTGTGTGTGTGTGTGTGTGTGTGTGTGTGTGTGTGT
AGAGGGGTGGGTTC[CG]GGGGGGTGGTGGAAGAGGGC[CG]AGG
GGGGAGGATAGAAGGAGGGGGTAGAGTTTCAGGG[CG]GGGAGGG
GGG[CG]CTGGGG[CG]CAGTGA[CG]GGAACCAATGAGCTGCCA
ACT[CG][CG][CG]TCTC[CG]G[CG]TGACTGC[CG]AGATTG
A[[CG]]TGGAGGACA[[CG]]TCAAATTGATTCC[CG]CA[CG]
CTGCAGCCTCC[CG]GTCAGA[CG]AATTTCTCCCAAT[CG]GAT
GAAGTTCACCCTGGGCCTGGGGT[[CG]][CG]GG[CG]TGGAGA
GTGTCCTGGGAGGGGGCAGCAG[CG]G[CG]G[CG]GCAGGCCCT
GGAG[CG]GG[CG]GCAG[CG][CG]CTC[CG]CTGC[CG][CG]
CACAG[CG][CG]TCTCCAGCC[CG][CG]GCTGGGC[CG]C[CG
][CG]GCTCT[CG]GCTCT[CG]GG[CG]CCCTCCCTCTATGCCT
CTCA[CG][CG]G[CG]G[CG]G[CG]G[CG]C
SEQ ID NO: 5124
GGGA[[CG]]TGTGTGTGTGTGTGTGTGTGTGTGTGTGTGTGTGT
GTGTGTGTAGAGGGGTGGGTTC[CG]GGGGGGTGGTGGAAGAGGG
C[CG]AGGGGGGAGGATAGAAGGAGGGGGTAGAGTTTCAGGG[CG
]GGGAGGGGGG[CG]CTGGGG[CG]CAGTGA[CG]GGAACCAATG
AGCTGCCAACT[[CG]][[CG]][CG]TCTC[CG]G[[CG]]TGA
CTGC[CG]AGATTGA[CG]TGGAGGACA[CG]TCAAATTGATTCC
[[CG]]CA[CG]CTGCAGCCTCC[CG]GTCAGA[CG]AATTTCTC
CCAAT[CG]GATGAAGTTCACCCTGGGCCTGGGGT[[CG]][CG]
GG[CG]TGGAGAGTGTCCTGGGAGGGGGCAGCAG[CG]G[CG]G[
CG]GCAGGCCCTGGAG[CG]GG[CG]GCAG[CG][CG]CTC[CG]
CTGC[CG][CG]CACAG[CG][CG]TCTCCAGCC[CG][CG]GCT
GGGC[CG]C[CG][CG]GCTCT[CG]GCTCT[CG]GG[CG]CCCT
CCCTCTATGCCTCTCA[CG][CG]G[CG]G[CG]G[CG]G[[CG]
]CCCAAGCTCTCC[CG]GACTG[CG]C[CG]GGCCCAGCCC[CG]
GCCACCC[CG]GCCACCC[CG]G[CG]CCAGGCAGCTGGC[CG]G
CC[CG]CT[CG]CTATGGGTAAGGGG[CG]GG[CG]ACAGGCAGG
GCT[CG]GGAGGGCTGAT
SEQ ID NO: 5125
AAAGCTAGAGGAGGAAGGGGGCTGAAAGGGGCTCAGAGCAGTGGA
AGGTCCCAGGCCCAGCTGGGGTTGGGA[[CG]]TGTGTGTGTGTG
TGTGTGTGTGTGTGTGTGTGTGTGTGTGTGTAGAGGGGTGGGTTC
[CG]GGGGGGTGGTGGAAGAGGGC[CG]AGGGGGGAGGATAGAAG
GAGGGGGTAGAGTTTCAGGG[CG]GGGAGGGGGG[CG]CTGGGG[
CG]CAGTGA[CG]GGAACCAATGAGCTGCCAACT[[CG]][[CG]
][CG]TCTC[CG]G[CG]TGACTGC[CG]AGATTGA[[CG]]TGG
AGGACA[CG]TCAAATTGATTCC[CG]CA[CG]CTGCAGCCTCC[
CG]GTCAGA[CG]AATTTCTCCCAAT[CG]GATGAAGTTCACCCT
GGGCCTGGGGT[[CG]][CG]GG[CG]TGGAGAGTGTCCTGGGAG
GGGGCAGCAG[CG]G[CG]G[CG]GCAGGCCCTGGAG[CG]GG[C
G]GCAG[CG][CG]CTC[CG]CTGC[CG][CG]CACAG[CG][CG
]TCTCCAGCC[CG][CG]GCTGGGC[CG]C[CG][CG]GCTCT[C
G]GCTCT[CG]GG[CG]CCCTCCCTCTATGCCTCTCA[CG][CG]
G[CG]G[CG]G[CG]G[CG]CCCAAGCTCTCC[CG]GACTG[CG]
C[CG]GGCCCAGCCC[CG]GCCACCC[CG]GCCACCC[CG]G[CG
]C
SEQ ID NO: 5126
GAGCCTTTACCTGCCAGGCTGCC[CG]GCCTCTGGAGGGTGGC[C
G]TGGCATACAGCTCCTG[CG]AGTGCTTGCTGTACTGCT[CG]G
A[CG][CG]TGGACCAGG[CG]CTTGGTGGG[CG]ACAGGGTGG[
[CG]]TA[CG]TGC[CG]ATGGTGGAGCTCAGCTGGTGGATGGG[
CG]AGGAGGAGATGGTGGACTGCA[CG]GTGGGGGG[CG]AGGTC
A[CG][CG]GAT[CG]GGGACAGGC[CG]GC[CG]AGGACA[CG]
A[CG]ATGTTGATGGG[CG]AGCTGGTGCTGTAGGACTTGGCCAG
G[CG]GCTGGG[CG]ACTGCTTGGG[CG]AGGAGC[[CG]][[CG
]][CG]G[CG][CG]G[CG]TAGGTGG[CG]CCCT[CG]GGGGC[
CG]AGC[CG]C[CG][CG]CTGCAGCTTGGTGGG[CG]AAC[CG]
CCCTGGGG[CG][CG]GCCAG[CG]GGGAGCCCC[CG][CG][CG
]G[CG]G[CG][CG]GGCAG[CG]TGGAGCTGGAGTAGTAGAG[C
G][CG]G[CG]G[CG]G[CG]G[CG]G[CG]GG[CG]G[CG][CG
]T[CG]GGCAGGTGGAAGG[CG]CTGCCCAGGCTGGG[CG][CG]
AA[CG]GCTCC[CG][CG]G[CG]G[CG]G[CG]G[CG]G[CG]G
[CG]G[CG][CG]GGCT[CG]GGCCC[CG]CCAGGTGGC[CG]G[
CG][CG]GCTGGT[CG]TGCCCTGTGCC[CG]GGAGAGGAGAGAA
CAC
SEQ ID NO: 5127
A[CG]AGTACTAGTACTAACACACTAATCAATTTGAGATGACTTC
CCCCTCATTCCCAAAGCTAGAGGAGGAAGGGGGCTGAAAGGGGCT
CAGAGCAGTGGAAGGTCCCAGGCCCAGCTGGGGTTGGGA[[CG]]
TGTGTGTGTGTGTGTGTGTGTGTGTGTGTGTGTGTGTGTGTGTAG
AGGGGTGGGTTC[CG]GGGGGGTGGTGGAAGAGGGC[CG]AGGGG
GGAGGATAGAAGGAGGGGGTAGAGTTTCAGGG[CG]GGGAGGGGG
G[CG]CTGGGG[CG]CAGTGA[CG]GGAACCAATGAGCTGCCAAC
T[CG][[CG]][CG]TCTC[CG]G[CG]TGACTGC[CG]AGATTG
A[[CG]]TGGAGGACA[CG]TCAAATTGATTCC[CG]CA[CG]CT
GCAGCCTCC[CG]GTCAGA[CG]AATTTCTCCCAAT[CG]GATGA
AGTTCACCCTGGGCCTGGGGT[[CG]][CG]GG[CG]TGGAGAGT
GTCCTGGGAGGGGGCAGCAG[CG]G[CG]G[CG]GCAGGCCCTGG
AG[CG]GG[CG]GCAG[CG][CG]CTC[CG]CTGC[CG][CG]CA
CAG[CG][CG]TCTCCAGCC[CG][CG]GCTGGGC[CG]C[CG][
CG]GCTCT[CG]GCTCT[CG]GG[CG]CCCTCCCTCTATGCCTCT
CA[CG][CG]G[CG]G[CG]G[CG]G[CG]CCC
SEQ ID NO: 5128
GGTCCTGGAGCATCCCCTC[CG][CG]TCCTTCCTCCCTCTGGGG
CTGGGGACAGC[CG]GGATGTCCCAGGCTGAGGTGGCCACCAGC[
CG]AG[CG][CG]GCTGCTAGGA[CG]CTGG[CG]TGGGGAG[CG
][CG]G[CG][CG]GAACTA[CG]GACAGTGAGCCCTGG[CG]CT
[CG]CTGCCCTG[CG]CCTTAATTTGCTGG[CG]G[CG]G[CG]A
TCC[[CG]]GAGGCC[CG]CAGCCAGTCAG[CG]C[CG]TCTCA[
CG]TCAC[CG]CTTCCTGATTC[CG]C[CG]C[CG]GGGG[CG]G
GGC[CG][CG]GGC[CG]GG[CG][CG]GAGGG[CG][CG]CCCA
GGGTG[CG]G[CG]CC[CG][CG]TGGCCTGT[CG]CCC[CG]GC
TGTT[[CG]]GTACCCCAGCACAGGTTCAGGGAAAAGGGTGCCAC
CACTAGGCTGA[CG]CAGCAGCCATGGACATCCCCACCTGGTCTC
ACAGCCC[CG]GG[CG]GGAGGG[CG]GTG[CG][CG]CTGT[CG
]GGAGAGGACACATCAC[CG][CG]GCCTAA[CG]CC[CG]AGAT
C[CG]GGCATGGCCCAGTTGGGGAAGCTATGACCAGACCAGTGGA
AGCCT[CG]GGAGTTCT[CG]CTTTTTCCATTCTTTTTCTTACCT
G[CG]CTCAAAGTTCCAAAAGAAAGTAGGGAAG[CG]CTCAGTAG
AA
SEQ ID NO: 5129
TCCTTCCTCCCTCTGGGGCTGGGGACAGC[CG]GGATGTCCCAGG
CTGAGGTGGCCACCAGC[CG]AG[CG][CG]GCTGCTAGGA[CG]
CTGG[CG]TGGGGAG[CG][CG]G[CG][CG]GAACTA[CG]GAC
AGTGAGCCCTGG[CG]CT[CG]CTGCCCTG[CG]CCTTAATTTGC
TGG[CG]G[CG]G[[CG]]ATCC[CG]GAGGCC[CG]CAGCCAGT
CAG[CG]C[CG]TCTCA[CG]TCAC[CG]CTTCCTGATTC[CG]C
[CG]C[CG]GGGG[CG]GGGC[CG][CG]GGC[CG]GG[CG][CG
]GAGGG[CG][CG]CCCAGGGTG[CG]G[CG]CC[CG][CG]TGG
CCTGT[CG]CCC[CG]GCTGTT[CG]GTACCCCAGCACAGGTTCA
GGGAAAAGGGTGCCACCACTAGGCTGA[CG]CAGCAGCCATGGAC
ATCCCCACCTGGTCTCACAGCCC[CG]GG[CG]GGAGGG[CG]GT
G[CG][CG]CTGT[CG]GGAGAGGACACATCAC[CG][CG]GCCT
AA[CG]CC[CG]AGATC[CG]GGCATGGCCCAGTTGGGGAAGCTA
TGACCAGACCAGTGGAAGCCT[CG]GGAGTTCT[CG]CTTTTTCC
ATTCTTTTTCTTACCTG[CG]CTCAAAGTTCCAAAAGAAAGTAGG
GAAG[CG]CTCAGTAGAATC[CG]G[CG]CCTCTGAGC[CG]G[C
G]CC
SEQ ID NO: 5130
AAACCAGCCATAGACCACT[CG]GGCCCCCT[CG]CCTCCTTTTT
TCCCTGGCTTGCACCCTC[CG]CACAG[CG]GGCTGCAGGTGCTT
CCCATTCAC[CG]CA[CG]GACCC[CG]CAGTGAAGG[CG][CG]
[CG]GCTGCAGC[CG][CG]CCTGCCTG[CG]TTGGGAAG[CG]G
[CG]AAATAGCCAAGT[CG]C[CG]CAGGGAAGGAAAC[CG]GCA
GGAAAACTGGAGGGGCACAGCCTGGAGCTTGCTACTCTCTAGGAT
CTGTTGTCAAAGCAGTTTGCAAAAACAGTCAGGCAAAACCTTGAG
G[CG]TA[CG]TA[CG]TTATCACCCA[CG]GATAT[CG][CG]A
AAGACACAGCTTAGCCCAGAGCCTATGCTAAGAACCCTCATCCAC
CATCTGAGGGGCAGGAGG[CG][CG]CAAGGTGTC[CG]AAGGGG
CATGGGAATTCCTATAAGAAA[CG]GTCCCTTCTGGATAGT[CG]
GAACATAGAGGAAAA[CG]GGGGTGGGTTAGGGGAACACTTACTC
TTGTGGGGCCCTG[CG]CTGG[[CG]]AATGTCAAGCCTCCAGGT
TCACAAATGGCTGGAGGTGCT[CG]GGGTTAGCTCC[CG]AGGG[
CG]GCAAG[CG]C[CG]AC[CG]GTCCT[CG]CT[CG]CTAGGCA
GG[CG]CCT
SEQ ID NO: 5131
CCTC[CG][CG]TCCTTCCTCCCTCTGGGGCTGGGGACAGC[CG]
GGATGTCCCAGGCTGAGGTGGCCACCAGC[CG]AG[CG][CG]GC
TGCTAGGA[CG]CTGG[CG]TGGGGAG[CG][CG]G[CG][CG]G
AACTA[CG]GACAGTGAGCCCTGG[CG]CT[CG]CTGCCCTG[CG
]CCTTAATTTGCTGG[CG]G[CG]G[CG]ATCC[CG]GAGGCC[C
G]CAGCCAGTCAG[CG]C[CG]TCTCA[CG]TCAC[CG]CTTCCT
GATTC[CG]C[CG]C[CG]GGGG[CG]GGGC[CG][CG]GGC[CG
]GG[CG][CG]GAGGG[CG][CG]CCCAGGGTG[CG]G[CG]CC[
CG][[CG]]TGGCCTGT[CG]CCC[CG]GCTGTT[[CG]]GTACC
CCAGCACAGGTTCAGGGAAAAGGGTGCCACCACTAGGCTGA[CG]
CAGCAGCCATGGACATCCCCACCTGGTCTCACAGCCC[CG]GG[C
G]GGAGGG[CG]GTG[CG][CG]CTGT[CG]GGAGAGGACACATC
AC[CG][CG]GCCTAA[CG]CC[CG]AGATC[CG]GGCATGGCCC
AGTTGGGGAAGCTATGACCAGACCAGTGGAAGCCT[CG]GGAGTT
CT[CG]CTTTTTCCATTCTTTTTCTTACCTG[CG]CTCAAAGTTC
CAAAAGAAAGTAGGGAAG[CG]CTCAGTAGAATC[CG]G[CG]CC
TCTGAG
SEQ ID NO: 5132
GTAGAGAATAATTATTGTACCAGGGCC[CG]C[CG]AGGCA[CG]
AGGCACTCTATTTTGTTTTGTAATCA[CG]A[CG]ACTATTATTT
TTAGTCTGATCAATGGGCACAATTTCTAAGCAG[CG]CAGTGGTG
GATGCT[CG]CAAACTTTTG[CG]CAC[CG]CTGGAAACCCACTA
GGTTGAGTTGCAAAA[CG]TAC[CG][CG]TAGA[CG]CCCCTGG
TGG[CG]C[CG]AGAGAAGAGCTAGGCCTGCCCAGCACAGAGC[C
G]GAGAG[CG]T[CG]GGCCTTC[CG]GAAGGGTAAGTTCTC[CG
]CCAAGGGGTCC[CG]AGGGAGCTGGA[[CG]]TCTGAATCTGGA
CTTGCCCCCAGCTT[CG]GGGTT[CG]ATTCTGGGTTTTG[CG][
CG]TCCCCAACCCCCAGGGCTTTC[CG]AAGCATGGCCTGGCTCC
AGGCC[CG]GTCCTGTAAGGACTGGAA[[CG]]GCAGCAAAATGT
GCAGGGAGGCAGT[CG]GC[CG]GCAGAGCTG[CG]G[CG]GGAG
CCAAGGTCAGGCC[CG][CG]GGGAGAG[[CG]]GGCAGCTTCCA
G[CG]C[CG]GCCACAAGCTCCCAGGCCAGCTGGGC[CG]CAGAC
CCCTTTGCTTCCAGAGAGCACAACC[CG][CG]TCCTTTCTCTCA
GCCAGGCTGCAGT
SEQ ID NO: 5133
GCCTCCTCCTC[CG]CTCTGCAGG[CG]GGGAC[CG]CC[CG]G[
CG]CT[CG]GCACC[CG]GCAG[CG][CG]GCCCCCTCCAGCCCC
[CG]GCTCC[CG]GCAGCAGAAGCAGAAGGCAG[CG]CCAGGGGC
[CG]C[CG]C[CG]C[CG]C[CG]AGCTC[CG][CG]GGGCT[CG
]GGAGC[CG]GCCC[CG]G[CG]AGGAGG[CG][CG]GAACCATG
GC[CG]ATGGGGG[CG]AGGG[CG]AAGA[CG]AGATCCAGTTCC
TG[CG]AACTGTAAG[CG]C[CG]TG[CG]T[CG][CG]TGTGCT
GTCAGGGGAAGGGGG[CG]TCAGGGCATCCACTAG[CG]GGGTC[
CG]GGCAGAGTGACAG[[CG]]GGCAG[CG]GGGACT[CG][CG]
GG[CG]GGG[CG]AGGGGGTGCCCCCTGAGGATG[[CG]]GGAGG
AG[CG]GGCATCACCAAGTGTGTGCAGGTGTG[CG]TGTTGGGG[
CG]AGGGAAGGCAAGGG[CG][CG]TGTCTGTG[CG][CG][CG]
TGTGGAAAGCTAGAGGATGGAG[CG][CG]GCTAGC[CG]G[CG]
GCAGG[CG]CC[CG]GGCT[CG]GACC[CG]GGGCAC[CG]GGGA
CAGGAG[CG]T[CG]GAGCTG[CG]GGAAC[CG]GGAGAGGAGGG
GA[CG]GC[CG]GTC[CG]GCCTGCCTGGTGGCA[CG]GCTGGGA
CCTCC[CG]GG[CG]GC[CG]GGGGCAGGG
SEQ ID NO: 5134
CTCCT[CG]C[CG]CTGT[CG]GGCT[CG]TCCTGCTGCTGCTGC
TGCTGCT[CG]T[CG][CG]C[CG]GGGCAGCCAGCTCAATGTGA
G[CG]AGCTGA[CG]C[CG]TCCAGCCATGCCAGTG[CG]CTC[C
G]GCAGCAGTA[CG][CG]CAGCAGTC[CG][CG]CAGCAGT[CG
]G[CG]TC[CG]CCTCCCAGTACCACCAGTGCCACAGCCTGCAGC
C[CG]C[CG]CCAGCCCCA[CG]GGCAGCCT[CG]GCAGTCTGGG
CTC[CG]GGCCCC[CG]CTCT[CG]CACCACCACCACCACC[CG]
CACC[CG]G[CG]CACCACCAGCACCACCAGCCCCAGG[CG][CG
]C[CG][CG]AGAGCAACCCCTTCAC[CG]AAATAGCCATGAGCA
GCTGCAGGTACAA[CG]GGGG[CG]TCATG[CG]GC[CG]CTCAG
CAACTTGAG[CG][CG]TCC[CG]C[CG]GAACCTGCA[CG]AGA
TGGACTCAGAGG[CG]CAGCCCCTGCAGCCCCC[CG][CG]TCTG
T[CG]GAGGAGGTGG[CG]G[CG][CG]TCCTCCC[CG]TCTGCA
GC[CG]CTGC[CG]C[CG]C[CG]C[CG]CTGTTT[CG]TCCTCA
GCCCC[CG]AGAT[CG]TGGTGTCTAAGCC[CG]AGCACAACAAC
TCCAACAACCTGG[CG]CTCTATGGAAC[CG]G[CG]G[CG]GAG
GCAGCACTGG
SEQ ID NO: 5135
CTCAGGCCACA[CG][CG]AGTCC[CG]TGGCCCAG[CG]GGTGC
C[CG]GGCAGGGG[CG]CTCCTCTG[CG][CG]CCC[CG]A[CG]
CCT[CG]C[CG]GCTCC[CG][CG]GCTCCTTACC[CG]C[CG]C
TCT[CG]GG[CG][CG]GGGCTC[CG]CAGCTAGGCCCCTGC[CG
]CC[CG]GTGGC[CG]G[CG]TC[CG][CG]GGGCCC[CG]GGCC
TCATACTCCTCTCCAGACAGGC[CG][CG]GGCTG[CG]GG[CG]
GGGTGGGC[CG]TGGATCC[CG]GG[CG]CCTAGGAC[CG]GGA[
CG][CG]GCAGCAAGG[CG]TGGAGAGAGCCCC[CG]GGGG[CG]
TCCCATGAGG[[CG]]TT[CG]GGAT[[CG]]G[CG]CCTCCTAG
GTG[CG]GGCTGTG[CG]GGCAGTCCTGCTCC[CG]CCCC[CG]G
GGCTGAGCTGG[CG]G[CG]T[CG]GCTTCAGAGCCT[CG]GAGC
[CG]AGC[CG]AGTC[CG]GGGAAG[CG]CTCCTTTCTCAGCTCC
AGC[CG]AAGAGC[CG]CC[CG]GCAGGGAGAGG[CG]GAGTCAC
TGG[CG]CC[CG]GGG[CG]GGGTGTTGGGGAGGGGTGAGTAGGG
ACAGGAGGGAGGGGGTGGAGAGTTGGGCTGCTG[CG]A[CG]CCT
CC[CG]GTGGCTCTGG[CG]GCACCAATG[CG]GGGCAC[CG]GA
GCAGGTGTC[CG]ACTG[CG]GTG
SEQ ID NO: 5136
GCAG[CG][[CG]]GCCCCCTCCAGCCCC[CG]GCTCC[CG]GCA
GCAGAAGCAGAAGGCAG[CG]CCAGGGGC[CG]C[CG]C[CG]C[
CG]C[CG]AGCTC[CG][CG]GGGCT[CG]GGAGC[CG]GCCC[C
G]G[CG]AGGAGG[CG][CG]GAACCATGGC[CG]ATGGGGG[CG
]AGGG[CG]AAGA[CG]AGATCCAGTTCCTG[CG]AACTGTAAG[
CG]C[CG]TG[CG]T[CG][CG]TGTGCTGTCAGGGGAAGGGGG[
CG]TCAGGGCATCCACTAG[CG]GGGTC[CG]GGCAGAGTGACAG
[CG]GGCAG[CG]GGGACT[CG][CG]GG[CG]GGG[CG]AGGGG
GTGCCCCCTGAGGATG[[CG]]GGAGGAG[CG]GGCATCACCAAG
TGTGTGCAGGTGTG[CG]TGTTGGGG[CG]AGGGAAGGCAAGGG[
CG][CG]TGTCTGTG[CG][CG][CG]TGTGGAAAGCTAGAGGAT
GGAG[CG][CG]GCTAGC[CG]G[CG]GCAGG[CG]CC[CG]GGC
T[CG]GACC[CG]GGGCAC[CG]GGGACAGGAG[CG]T[CG]GAG
CTG[CG]GGAAC[CG]GGAGAGGAGGGGA[CG]GC[CG]GTC[CG
]GCCTGCCTGGTGGCA[CG]GCTGGGACCTCC[CG]GG[CG]GC[
CG]GGGGCAGGGTGCAGAGCAGGGTGGCC[CG]TGGGGG[CG][C
G]GGGAG[CG]G[CG]GGAACT[CG]GG
SEQ ID NO: 5137
TTCCTCCTTCAAAACCAAACCAAACCACCACCAACAAAAACTTCT
GTGCTACTCTGGCTTCTCTGGTTTTGTTTGGGAACAGTGGAACCA
AGTTAGCC[CG]GGCCAGGG[CG]CAGATGATGGACTCAGAG[CG
]CCCAGGGACCCTAGAGAGAGGAGCACTCCTCAAGAGCCCCCTGG
CCATCACC[CG]AG[CG]CCCTGGAG[CG]CCATCACC[CG]AA[
CG][CG][CG]CTCCAGGCCCT[CG]AACAAGGCCTCTGGCTGCC
AGAG[CG]AGTGAGGGG[[CG]]CAGAGG[CG]GCAGAGAG[CG]
GAGAGCCC[CG]GTGTCTC[CG][CG]AGGG[CG]G[CG]G[CG]
GCCAGCAGA[CG]G[CG]AT[CG]AGG[CG][CG][CG]CCA[CG
]GCA[CG]GCCAG[CG]CAGACA[CG]C[CG][CG]GGGTCT[CG
]GGC[CG]GAGC[CG]TGCAGC[CG]GGCC[CG]CTGCCTCTTTG
CCCCTCATGGCTC[CG][CG][CG]GGAGGAAAC[CG]GGCCTTC
TC[CG]CC[CG]CCCTCCTCT[CG]CTG[CG]GTGTCCCCAGCAC
CCC[CG]CCC[CG]CCCC[CG][CG][CG]GTCCCCAC[CG]T[C
G][CG]CTCCCCTCCCTCCT[CG]CCCT[CG]CTC[CG][CG]CC
TCCTGTTGGCAGAGATGGCCT[CG]AAGGCCTCTTCCAATTTGAA
SEQ ID NO: 5138
CACACACCCCCTTTCCCTGGGCTGTTATTATTTTTTTTTCCTCCT
TCAAAACCAAACCAAACCACCACCAACAAAAACTTCTGTGCTACT
CTGGCTTCTCTGGTTTTGTTTGGGAACAGTGGAACCAAGTTAGCC
[CG]GGCCAGGG[CG]CAGATGATGGACTCAGAG[CG]CCCAGGG
ACCCTAGAGAGAGGAGCACTCCTCAAGAGCCCCCTGGCCATCACC
[CG]AG[CG]CCCTGGAG[CG]CCATCACC[CG]AA[CG][CG][
CG]CTCCAGGCCCT[CG]AACAAGGCCTCTGGCTGCCAGAG[CG]
AGTGAGGGG[[CG]]CAGAGG[CG]GCAGAGAG[CG]GAGAGCCC
[CG]GTGTCTC[[CG]][CG]AGGG[CG]G[CG]G[CG]GCCAGC
AGA[CG]G[CG]AT[CG]AGG[CG][CG][CG]CCA[CG]GCA[C
G]GCCAG[CG]CAGACA[CG]C[CG][CG]GGGTCT[CG]GGC[C
G]GAGC[CG]TGCAGC[CG]GGCC[CG]CTGCCTCTTTGCCCCTC
ATGGCTC[CG][CG][CG]GGAGGAAAC[CG]GGCCTTCTC[CG]
CC[CG]CCCTCCTCT[CG]CTG[CG]GTGTCCCCAGCACCCC[CG
]CCC[CG]CCCC[CG][CG][CG]GTCCCCAC[CG]T[CG][CG]
CTCCCCTCCCTCCT[[CG]]CCCT[CG]CTC[CG][CG]CCTCCT
GT
SEQ ID NO: 5139
GAATTGGCTAGAGGAGGGATGAGGTGACATTTGGTCTCTGAATG[
CG]TCT[CG]GATCC[CG]GAC[CG][CG]AGATCCAT[CG]CTC
CCAGTG[CG]TGCAGTTATTC[CG]TCCTGCTGTGTGACA[CG]G
GAGAATGG[[CG]][CG]CA[CG]CCTGTGCACCTGCAT[CG]AC
[CG]TGTAGCTGCCTGGGTGACAGCCACCCATTTGTCTATGTTTT
CTTTCCTATTCTAAAGTAAA[CG]ATTAGAAGTGGCCTCATAGAG
TCTTAGGAAGAAACAGTAAGCCCTGTGCCACC[CG]GGGTCACTG
CCTAGCCCTGCCCTC[CG][CG][[CG]]CAC[CG]TC[CG][CG
]TCTC[CG]AGGGCTCCTCCT[CG]GACCTGGACCCTCTGGAG[C
G]GCTT[CG]ACCCTCCCTCTGCCCC[CG]CTGCCTG[CG]TCCA
GACCCCCTCC[CG]GGTGGGCTCCTC[CG]AGGACCTC[CG]CAG
C[CG][CG]GG[CG]AGCTGGGTGCCTG[CG]AGT[CG]C[CG]C
[CG]CAGTCTGAGCCTCTGGTGCCCCTTAG[CG]TTGGCTCT[CG
]GAGTTCTGAGAAGGGAAAGGAGACAGACACTCTGGGAAAATCTG
GGGTCACCT[CG]ATTC[CG]GCTCCTA[CG]CTGGATGC[CG]T
GGGCAAGCAT[CG]GTCTC
SEQ ID NO: 5140
CATTGGTCCAAA[[CG]]GTGTCAGTGAACTTTAGCTGCAAAGAA
ACTACCTCATTTAAAATTTAAGCAGACCACAGGGAGTAGCATTGG
AGGAAAGAGAGGAGATTTTCTCCTTCAACATTTTGAGCATACTGG
AATGCCAACCCCCTAGGTAGGTGCCAGGGCCACCACAGGAACCTG
ACT[[CG]]CAGGAGT[CG]TCCTC[CG]TCTCTA[CG][CG]AA
G[CG]CCCCCT[CG]AGGTGGGCAGGGGGTGG[CG]G[CG]G[CG
]A[CG]GTGG[CG]GAGC[CG]CAGAG[CG]AGCTAGAGAG[CG]
AGAG[[CG]]GTGAGACTCTG[CG]GA[[CG]]TCTTCC[CG]CC
[CG]C[CG][CG]CTC[CG]CCCCACCCAGCCTC[CG]CAGCCT[
CG]GGC[CG]GAT[CG]CC[CG]G[CG]GGGAAGATG[CG]CCTC
AAGAACCAGGTAGAAG[CG]CCT[CG]G[[CG]]CAGGCCC[CG]
CACCCC[CG]ACTCTGCC[CG]CCCTCTGTTA[CG]GACACC[CG
]CTGGGCCA[[CG]]TGGT[CG][CG]ACTGGCTTCTCCCAG[CG
]GCCAGCCTGGCCACCC[CG]ACTCCCAGGGAGGGGGAGAGGGCC
CTTGCAAGAGGATCCTGCCCC[CG][CG]CTGCCCAGACCTGAGG
GCTCCTGG[CG]ACCCCACC[CG]CAGCCCATC
SEQ ID NO: 5141
TGTGCCACC[CG]GGGTCACTGCCTAGCCCTGCCCTC[CG][CG]
[[CG]]CAC[CG]TC[CG][CG]TCTC[CG]AGGGCTCCTCCT[C
G]GACCTGGACCCTCTGGAG[CG]GCTT[CG]ACCCTCCCTCTGC
CCC[CG]CTGCCTG[CG]TCCAGACCCCCTCC[CG]GGTGGGCTC
CTC[CG]AGGACCTC[CG]CAGC[CG][CG]GG[CG]AGCTGGGT
GCCTG[CG]AGT[CG]C[CG]C[CG]CAGTCTGAGCCTCTGGTGC
CCCTTAG[CG]TTGGCTCT[CG]GAGTTCTGAGAAGGGAAAGGAG
ACAGACACTCTGGGAAAATCTGGGGTCACCT[CG]ATTC[[CG]]
GCTCCTA[CG]CTGGATGC[CG]TGGGCAAGCAT[CG]GTCTCCT
TTGATCTGAAGATCCTGCCCACTC[CG]CCCCTTGCCCCTGCCAG
ACC[CG][CG]GGAGCAGCAGGTGG[CG]TGGGTGCCAGA[[CG]
]CTGC[CG]GTG[CG]CAC[CG]AGTGCAG[CG]CTGCCC[CG]G
CT[CG]CCC[CG]GGGGT[CG][CG]TGGGACAG[CG]GTTTG[C
G]GTGT[CG]CCCTCTCTG[CG]GTGCTTTCCAAGGACCCTCATG
CCC[CG]CC[[CG]]GGTGCACATTCACCACATGGGTCTTACTAG
GTGACCTCTTTCTAAGTAAAATGCATTGAGAAA
SEQ ID NO: 5142
TGGCAG[CG][CG]GAGGCTTGGAGAGAAGCCCCATGCTGGCTCC
CATTCAGC[CG]GCC[[CG]]TTTTCCT[CG]AGCTTTGGAAGTT
TCACTCAGC[CG]TGCACTCAATGGCTTCACAAAGCTGATTACAA
GCTTCAG[CG]CATTCCTGAAGGAGCCAAAAG[CG]A[CG]CAGG
TGCAAA[[CG]]AGC[CG]AGGGAGCCCCTTATCC[CG]GTGACA
GAATGGGACAAGCTGGGAAAGGCTTAGACCACACAAGTCCAAGGC
TCACCAGGC[CG]CAGAAAGCCTGCCTTGGGAAC[CG]GGGGTCA
TTATC[CG]CCCTATTCAG[CG]GGGCC[CG]GGGACCCTGGGGG
C[CG]AG[CG]AGGCCAGCC[CG]GG[CG]GGAGCAC[CG]C[CG
]CCAG[CG][CG][CG]C[CG]G[CG]ACAGGGGGGAACCC[CG]
CTGGG[CG]CTG[CG]GCCAGGC[CG]GGGCTCAGGG[CG]CTGG
GCTGTG[CG]TTTGCACAAATCTGTGTTC[CG]G[CG]GGC[CG]
GTGTCAACCCTAGTGGGGACA[CG]GGAGAAAGAGCA[CG]CCAG
TAGGCCA[CG][CG]G[CG][CG]TGGGCAGTG[CG]CAACTTCT
GT[CG]G[CG]TCCAGGCTGTA[CG]GCCA[CG]TTTCAGC[CG]
GTGCCCCCAGGCCATGGACACCCAGCT
SEQ ID NO: 5143
GGTG[CG]GG[CG]GAGGC[CG]G[CG]GGGGG[CG]C[CG]GGG
G[CG]CTG[CG][CG]GCCCTTGG[CG]G[CG]GGGG[CG][CG]
[CG]GGGTGG[CG]GGGGAGGC[CG]AGG[CG]C[CG]GCAGCTT
[CG][CG]C[CG]G[CG]GCTGGAAG[CG]GG[CG]GGCTGCA[C
G]GG[CG]GCT[CG]AGTG[[CG]]GGGACCCCAGCCCCT[CG]C
CCT[CG]TGAG[CG]C[CG]CCCCTGCCACCTGCTGCCAAGTCAC
[CG]GACTGGGGATG[CG]GGGGG[CG]GGGTG[CG]GGGAGAGG
AAAG[CG]C[CG][CG]GGTGGCA[CG]GTGCCTCTGG[CG]ACA
GAGTAGATGGACC[CG]AAAGAGACC[[CG]]AGGTGACTT[CG]
GGCTGGGCAGGACACCCC[CG]GGAGTTGCCAG[[CG]]GCTCTC
AG[CG]GG[CG]CCTCTAGGACCCCTTGTCACTG[CG]AGCAG[C
G]CCTGCTCTGGGGAGGGGCT[CG]AAGG[CG]C[CG][CG]CCT
TCTCAA[CG][CG]C[CG]CTG[[CG]]TCCCCCAGGGGCTCCAA
CTTGGACAGCTAGACTGGAAATACTGCTGCACAAAGTTAGGCTGA
GTT[CG]AAGGACACCAGAGAGGAAG[[CG]]CAAGGAGATGCCC
TTCTC[CG]GAGC[CG]GGCAGAG[CG]CCAGAACTCCTAGA[CG
]GGAAACTTTCCCCCATT
SEQ ID NO: 5144
AGCCCACAACTAGGCTGC[CG]GGAC[CG]GGGAGCCAGACCCCA
GGT[CG]CTCCAACTC[CG]G[CG]AGGG[CG]GGGAGGGGAGTG
ACAGCAGCAACTAG[CG][CG][CG]CTGGCAAAAGCACAGAGTC
T[[CG]]GAATAGGCTAAA[CG]GC[CG]G[CG][CG]GAGGGGT
GGAGGCAAGAGGGGG[CG]GAGGAGGAGTTGCC[CG]AG[CG]GA
GAGGGATGA[CG]TCCTCCAGGGCCTTGGGCTC[CG]AGGAAC[C
G]AAGACTGGTG[CG]CT[CG]C[CG]GGCCC[CG][CG]CACTA
GCTCTGGGGTT[CG]CAGAGG[CG]C[CG][CG]CC[CG]GCTTC
[CG][CG]GCAGCTCCAACACAG[CG][CG]CAGG[CG]GT[CG]
ATCC[CG]GTCAGCTTAAAC[CG]GG[CG]CAAGCCCTCTGTCCC
CCTCAAAGCACCTCTCTGCT[CG]TCCCTAGTCTCCAGGTGCC[C
G]TCTGAGAGTCCCTGT[CG]CCTC[CG]GG[CG]GACC[CG]GC
CAGGCAGCCCC[CG]GT[CG]GGCCAGCCT[CG]CCCCTTCCT[C
G]CTTTCTCCCCCAT[CG]ACCT[CG]AGCCAAGGGCAGG[CG]G
CC[CG]CACC[CG][CG]CCCCTGCCCA[CG]GGGCTAGG[CG]G
[CG]CTCC[CG]GCCTA[CG]CC[CG]GCTGCCAAGAAGAGAAAG
G[CG]C
SEQ ID NO: 5145
[CG]ATTGTGAGGGGCTCTGCTGGAATTTGGCAG[CG][CG]GAG
GCTTGGAGAGAAGCCCCATGCTGGCTCCCATTCAGC[CG]GCC[[
CG]]TTTTCCT[CG]AGCTTTGGAAGTTTCACTCAGC[CG]TGCA
CTCAATGGCTTCACAAAGCTGATTACAAGCTTCAG[CG]CATTCC
TGAAGGAGCCAAAAG[CG]A[CG]CAGGTGCAAA[CG]AGC[CG]
AGGGAGCCCCTTATCC[CG]GTGACAGAATGGGACAAGCTGGGAA
AGGCTTAGACCACACAAGTCCAAGGCTCACCAGGC[CG]CAGAAA
GCCTGCCTTGGGAAC[CG]GGGGTCATTATC[CG]CCCTATTCAG
[CG]GGGCC[CG]GGGACCCTGGGGGC[CG]AG[CG]AGGCCAGC
C[CG]GG[CG]GGAGCAC[CG]C[CG]CCAG[CG][CG][CG]C[
CG]G[CG]ACAGGGGGGAACCC[CG]CTGGG[CG]CTG[CG]GCC
AGGC[CG]GGGCTCAGGG[CG]CTGGGCTGTG[CG]TTTGCACAA
ATCTGTGTTC[CG]G[CG]GGC[CG]GTGTCAACCCTAGTGGGGA
CA[CG]GGAGAAAGAGCA[CG]CCAGTAGGCCA[CG][CG]G[CG
][CG]TGGGCAGTG[CG]CAACTTCTGT[CG]G[CG]TCCAGGCT
GTA[CG]GCCA[CG]TTTCAGC[CG]G
SEQ ID NO: 5146
TCAGCAGCAAACCCCTCTCAACTG[CG]TGGCC[CG][CG]ATCA
ACACTCTCATCTTTGCTTAGTAATGATACATTTCCACCACACATT
TTCTTATACTGCCTTTTACT[CG]ATGACT[CG]TGATACTATTT
TCCTCTCATTCCTTCCCACCTCTACACTGCC[CG][CG]TTACCA
CACACCCATAAAG[CG]ACATCAG[CG]GTCTCCAGGG[CG]GAA
AGGGGTGGAAGCTGACCCT[CG]CCCTTCCCTCCAG[CG]CTGGC
TTCAGGTGTGCCTTCTGCTACCTCCTGTACTG[CG]AACAGGGGC
C[CG]C[CG]AGCTC[CG]GGAGCCCCTAGAAGAGGAAGACTCCT
CTGGCCCCACTAGGTATCATC[CG][CG]CTCTCC[CG]CTTTCC
ACCTG[CG]CCCT[CG]CTTGGGCCAATCTCTGC[CG]CA[CG]T
GTCCATCCCTGAACTGCA[CG]CTATCCTCCACCCC[CG]GGGGG
TTCCTG[CG]CACTGAAAGAC[CG]TTCTC[CG]GCAGGTTTTGG
GATC[CG]G[CG]A[CG]GCTGAC[CG][CG][CG]C[CG]CCCC
CA[CG]CC[CG]GTTCCA[CG]ATGCTGCAATACAGAAAGTTTA[
CG]T[CG]GCCC[CG]ACC[CG][CG][CG]GGACTGCAGGGTC[
CG]C[CG]GAG[CG][CG]GC
SEQ ID NO: 5147
CACACACATGCACACATTTTCT[CG][CG]CTCTCTC[CG]GCTC
TCCTTTGTTTATTTTCTAATCTATATTTTTACTGGAAGATTTCCT
CTTTATTCTCTCC[CG]CCCTCCTACAAG[CG]CTCTTGCTGGC[
CG]TCTGGGTGCACACAC[CG]CTCCCT[CG]ATCACCCCAGCCC
CCTTCCTGGTCTCC[CG]AG[CG][CG]GGGTTTGAAGGTCACCT
CCTTTCCAGTCCC[CG]TG[CG]AGC[CG][CG]CTGC[CG]C[C
G]CCTCCTCCAGCCAGAGT[CG]GTGGGACTGGCTG[CG]CTGCC
CTGAAGTGGTTCTCCAAGCAG[[CG]][CG]GAGGGTGG[[CG]]
GA[CG]G[CG]GA[CG]GAGCCCAGGGGC[CG][CG]T[CG]GGT
GGGGAAACC[CG]AACT[CG][CG]GAGGGGAATCCCTCCCCCTC
[CG]CCCCAGCCCCCCAGCAGCACC[CG][CG]GTGGGG[CG]GG
GG[CG]CTCTGCCAGCCC[CG]GGAACAGCAGAGG[CG]G[CG]G
CACTGGCTGGACCCA[CG][CG][CG][CG]CCTC[CG]GGGCTG
AAGAAGGAAGGAGTGAGC[CG]AGC[CG]AGCACCCCACATCTGG
AGGGGACAGCCAGC[CG]TGGGCCC[CG]CCC[CG]G[[CG]]TC
[CG]GAGCAGGAGAACTC[CG]AGCTTCTTGCCCA
SEQ ID NO: 5148
CACACATTTTCT[CG][CG]CTCTCTC[CG]GCTCTCCTTTGTTT
ATTTTCTAATCTATATTTTTACTGGAAGATTTCCTCTTTATTCTC
TCC[[CG]]CCCTCCTACAAG[CG]CTCTTGCTGGC[CG]TCTGG
GTGCACACAC[CG]CTCCCT[CG]ATCACCCCAGCCCCCTTCCTG
GTCTCC[CG]AG[CG][CG]GGGTTTGAAGGTCACCTCCTTTCCA
GTCCC[CG]TG[CG]AGC[CG][CG]CTGC[CG]C[CG]CCTCCT
CCAGCCAGAGT[CG]GTGGGACTGGCTG[CG]CTGCCCTGAAGTG
GTTCTCCAAGCAG[CG][CG]GAGGGTGG[[CG]]GA[CG]G[CG
]GA[CG]GAGCCCAGGGGC[CG][CG]T[CG]GGTGGGGAAACC[
CG]AACT[CG][CG]GAGGGGAATCCCTCCCCCTC[CG]CCCCAG
CCCCCCAGCAGCACC[CG][CG]GTGGGG[CG]GGGG[CG]CTCT
GCCAGCCC[CG]GGAACAGCAGAGG[CG]G[CG]GCACTGGCTGG
ACCCA[CG][CG][CG][CG]CCTC[CG]GGGCTGAAGAAGGAAG
GAGTGAGC[CG]AGC[CG]AGCACCCCACATCTGGAGGGGACAGC
CAGC[CG]TGGGCCC[CG]CCC[CG]G[[CG]]TC[CG]GAGCAG
GAGAACTC[CG]AGCTTCTTGCCCAGGCAGAGAGA
SEQ ID NO: 5149
CACACACACATGCACACATTTTCT[CG][CG]CTCTCTC[CG]GC
TCTCCTTTGTTTATTTTCTAATCTATATTTTTACTGGAAGATTTC
CTCTTTATTCTCTCC[CG]CCCTCCTACAAG[CG]CTCTTGCTGG
C[CG]TCTGGGTGCACACAC[CG]CTCCCT[CG]ATCACCCCAGC
CCCCTTCCTGGTCTCC[CG]AG[CG][CG]GGGTTTGAAGGTCAC
CTCCTTTCCAGTCCC[CG]TG[CG]AGC[CG][CG]CTGC[CG]C
[CG]CCTCCTCCAGCCAGAGT[CG]GTGGGACTGGCTG[CG]CTG
CCCTGAAGTGGTTCTCCAAGCAG[[CG]][[CG]]GAGGGTGG[[
CG]]GA[CG]G[CG]GA[CG]GAGCCCAGGGGC[CG][CG]T[CG
]GGTGGGGAAACC[CG]AACT[CG][CG]GAGGGGAATCCCTCCC
CCTC[CG]CCCCAGCCCCCCAGCAGCACC[CG][CG]GTGGGG[C
G]GGGG[CG]CTCTGCCAGCCC[CG]GGAACAGCAGAGG[CG]G[
CG]GCACTGGCTGGACCCA[CG][CG][CG][CG]CCTC[CG]GG
GCTGAAGAAGGAAGGAGTGAGC[CG]AGC[CG]AGCACCCCACAT
CTGGAGGGGACAGCCAGC[CG]TGGGCCC[CG]CCC[CG]G[[CG
]]TC[CG]GAGCAGGAGAACTC[CG]AGCTTCTTGCC
SEQ ID NO: 5150
GGTGCC[CG]GGAAC[CG]CT[CG]C[CG]AAGGAGGAAG[CG]C
[CG]TC[CG]TCTGTCCTTCCC[CG]G[CG]G[CG]G[CG]G[CG
]AG[CG]GAG[CG]CAGGAGCCC[CG]CACTCCACACACTCATGC
ACACACACACTCACACACACTCACACACTCTCCCACAACACAATA
CCCTGACACACACTCACA[CG]CA[CG]C[CG]CACTCACAC[CG
]GGTGCCTGGGAAAAT[CG]CAGA[CG]C[CG]GGGAGGAGCAGG
GGG[CG]GTGATGGGAAGGGGAGCTG[CG]AGG[CG]AAGTGTTC
TTCAGGGAAG[CG]GGCT[CG]AGTCTC[[CG]]CAGCTG[CG]G
[[CG]]G[CG]G[CG]G[CG]G[CG][CG]CTGGGC[CG]G[CG]
G[CG]GG[CG][CG]GGCAGGGGGC[CG]GGGGTGC[CG][CG][
CG]GTAGGAGCCTGGCTTGGACAGC[[CG]]AGGAGCAGGAAGTG
GCCTCTGACCCAGGACAC[[CG]]AGCAGGGCTCTCTTGCCTGGA
TT[CG]CCTTTACAAAGTAACT[CG][CG]AAGGTCCC[CG]GAG
GGG[CG]TCAAG[CG]G[CG]ACAG[CG]GCC[CG]CC[CG]CC[
CG]CAGTC[CG]AAGCCCCCAGCCCTGCTGTCTTGCCTTCCCCCA
TTCCATCAGTTGCCATTGCAA[CG]CCAATCCTGTTACA[CG]AT
ACAGACTC
SEQ ID NO: 5151
GGCCAAGC[CG]AGGTGCC[CG]GGAAC[CG]CT[CG]C[CG]AA
GGAGGAAG[CG]C[CG]TC[CG]TCTGTCCTTCCC[CG]G[CG]G
[CG]G[[CG]]G[CG]AG[CG]GAG[CG]CAGGAGCCC[CG]CAC
TCCACACACTCATGCACACACACACTCACACACACTCACACACTC
TCCCACAACACAATACCCTGACACACACTCACA[CG]CA[CG]C[
CG]CACTCACAC[CG]GGTGCCTGGGAAAAT[CG]CAGA[CG]C[
CG]GGGAGGAGCAGGGGG[CG]GTGATGGGAAGGGGAGCTG[CG]
AGG[CG]AAGTGTTCTTCAGGGAAG[CG]GGCT[CG]AGTCTC[[
CG]]CAGCTG[CG]G[[CG]]G[CG]G[CG]G[CG]G[CG][CG]
CTGGGC[CG]G[CG]G[CG]GG[CG][CG]GGCAGGGGGC[CG]G
GGGTGC[CG][CG][CG]GTAGGAGCCTGGCTTGGACAGC[[CG]
]AGGAGCAGGAAGTGGCCTCTGACCCAGGACAC[CG]AGCAGGGC
TCTCTTGCCTGGATT[CG]CCTTTACAAAGTAACT[CG][CG]AA
GGTCCC[CG]GAGGGG[CG]TCAAG[CG]G[CG]ACAG[CG]GCC
[CG]CC[CG]CC[CG]CAGTC[CG]AAGCCCCCAGCCCTGCTGTC
TTGCCTTCCCCCATTCCATCAGTTGCCATTGCAA[CG]CCAATCC
TGTTACAC
SEQ ID NO: 5152
G[CG][CG]GTCAGCA[CG]AGGACCAGCA[CG]ACCA[CG]ACC
TTGG[CG]TTCATGG[CG][CG]GG[CG]GG[CG]GG[CG]GG[C
G]GG[CG]GA[CG]AG[CG][CG]GGT[CG]GGGGC[CG]GA[CG
]C[CG]AG[CG]GGCAATG[CG]GCTGA[CG]GAGAGTGAAAGTG
[CG]G[[CG]]GTGGGAGG[CG][CG][CG]C[CG]GG[CG]CTT
TAGAGGGGAGAGCCAGG[CG]GGG[CG]GGG[CG]GGG[CG][CG
][CG]GTGAGGGG[CG]GGCC[CG]GAGC[CG]AGTGCTGGC[CG
]TGGC[CG]GGGTCTCTGA[CG][CG][CG]ACTCCCT[CG]G[C
G]GCACCCCCCACCCATCCCCTT[CG]CCCCTCCTC[[CG]]CTC
CCTCTGTGCTCC[CG]AGG[CG]CAGTG[CG]CTC[CG]GCCTTT
GACCTTCTCAGGCTC[CG]CTGGGCCC[CG]CAC[CG]CTGGCC[
CG]GTCTGCAGTGAGGTC[CG]CAGCCTGAG[CG]A[[CG]]C[C
G]GCTGGAGGGC[CG]CTTATTGTCCCTGTGACAGGGCCCCACTG
GAGAGACTGAGGACCTTAGG[CG]TAAAGTGGGG[CG][CG][CG
]TGGAAAGCTG[CG]GGA[CG]GGGCCTTT[CG]ATGG[CG]GGA
ACTGAATGAGAACCAATGAAAAGCAAACAAGTTAGTCC[CG]GG[
CG]GAGGGCCTGCTGG[CG]GTGG[CG]GCCC
SEQ ID NO: 5153
CA[CG]ACCA[CG]ACCTTGG[CG]TTCATGG[CG][CG]GG[CG
]GG[CG]GG[CG]GG[CG]GG[CG]GA[CG]AG[CG][CG]GGT[
CG]GGGGC[CG]GA[CG]C[CG]AG[CG]GGCAATG[CG]GCTGA
[CG]GAGAGTGAAAGTG[[CG]]G[CG]GTGGGAGG[CG][CG][
CG]C[CG]GG[CG]CTTTAGAGGGGAGAGCCAGG[CG]GGG[CG]
GGG[CG]GGG[CG][CG][CG]GTGAGGGG[CG]GGCC[CG]GAG
C[CG]AGTGCTGGC[CG]TGGC[CG]GGGTCTCTGA[CG][CG][
CG]ACTCCCT[CG]G[CG]GCACCCCCCACCCATCCCCTT[CG]C
CCCTCCTC[CG]CTCCCTCTGTGCTCC[CG]AGG[[CG]]CAGTG
[CG]CTC[CG]GCCTTTGACCTTCTCAGGCTC[CG]CTGGGCCC[
CG]CAC[CG]CTGGCC[CG]GTCTGCAGTGAGGTC[CG]CAGCCT
GAG[CG]A[CG]C[CG]GCTGGAGGGC[CG]CTTATTGTCCCTGT
GACAGGGCCCCACTGGAGAGACTGAGGACCTTAGG[CG]TAAAGT
GGGG[CG][CG][CG]TGGAAAGCTG[CG]GGA[CG]GGGCCTTT
[CG]ATGG[CG]GGAACTGAATGAGAACCAATGAAAAGCAAACAA
GTTAGTCC[CG]GG[CG]GAGGGCCTGCTGG[CG]GTGG[CG]GC
CCAAGGCAGC[CG]AGGGGGTCCTGC
SEQ ID NO: 5154
GAAA[CG]GTCCCTTCTGGATAGT[CG]GAACATAGAGGAAAA[C
G]GGGGTGGGTTAGGGGAACACTTACTCTTGTGGGGCCCTG[CG]
CTGG[CG]AATGTCAAGCCTCCAGGTTCACAAATGGCTGGAGGTG
CT[CG]GGGTTAGCTCC[CG]AGGG[CG]GCAAG[CG]C[CG]AC
[CG]GTCCT[CG]CT[CG]CTAGGCAGG[CG]CCTC[CG]GATTC
[CG]TTCATGCCTCT[CG]GAGCTACAGGGCTTGGTG[CG]GAGA
[CG]CAGGG[CG]GG[[CG]][CG]CTGGGCTCTGGGTGTC[CG]
CAACCCAAAGTGCAGCTGGTGTCTTGA[CG]CTAC[[CG]]TCTA
TGCACAAGC[CG]G[CG]G[CG]G[CG]G[CG]GAGCTTGGAGGA
GGCAGGGAGATTCCAAATAAATCCACTAATTCTGGGCTGAG[CG]
CTC[[CG]]G[CG]TG[CG]AGCC[CG]CCTGCCTGCCAAATGTA
ACTGTGAAGTGATGTGGAAAAGTGAGC[[CG]]GGTGCCTCCCCT
GA[CG]GATCTCATTGGGCAGAG[CG]CAGAGGCAGGTCTACCAC
TTGGAAATTCATGTAAGCAGTCCCCCCTCCC[CG]CCCCCTCCC[
CG]CCTTCCCCCTT[CG]CTCTCC[CG][CG]CTGGCT[CG]AT[
CG]CTCTCTTTCCCCCTACCCAA
SEQ ID NO: 5155
CATAGAGGAAAA[CG]GGGGTGGGTTAGGGGAACACTTACTCTTG
TGGGGCCCTG[CG]CTGG[[CG]]AATGTCAAGCCTCCAGGTTCA
CAAATGGCTGGAGGTGCT[CG]GGGTTAGCTCC[CG]AGGG[CG]
GCAAG[CG]C[CG]AC[CG]GTCCT[[CG]]CT[CG]CTAGGCAG
G[CG]CCTC[CG]GATTC[CG]TTCATGCCTCT[CG]GAGCTACA
GGGCTTGGTG[CG]GAGA[CG]CAGGG[CG]GG[[CG]][CG]CT
GGGCTCTGGGTGTC[[CG]]CAACCCAAAGTGCAGCTGGTGTCTT
GA[CG]CTAC[[CG]]TCTATGCACAAGC[CG]G[CG]G[CG]G[
CG]G[[CG]]GAGCTTGGAGGAGGCAGGGAGATTCCAAATAAATC
CACTAATTCTGGGCTGAG[CG]CTC[[CG]]G[CG]TG[CG]AGC
C[CG]CCTGCCTGCCAAATGTAACTGTGAAGTGATGTGGAAAAGT
GAGC[[CG]]GGTGCCTCCCCTGA[CG]GATCTCATTGGGCAGAG
[CG]CAGAGGCAGGTCTACCACTTGGAAATTCATGTAAGCAGTCC
CCCCTCCC[CG]CCCCCTCCC[CG]CCTTCCCCCTT[CG]CTCTC
C[CG][CG]CTGGCT[CG]AT[CG]CTCTCTTTCCCCCTACCCAA
GTGGG[CG]CACAACTTTT[CG]CTCCT[CG]C
SEQ ID NO: 5156
GGTGGATGCT[CG]CAAACTTTTG[CG]CAC[CG]CTGGAAACCC
ACTAGGTTGAGTTGCAAAA[CG]TAC[CG][CG]TAGA[CG]CCC
CTGGTGG[CG]C[CG]AGAGAAGAGCTAGGCCTGCCCAGCACAGA
GC[CG]GAGAG[CG]T[CG]GGCCTTC[CG]GAAGGGTAAGTTCT
C[CG]CCAAGGGGTCC[CG]AGGGAGCTGGA[CG]TCTGAATCTG
GACTTGCCCCCAGCTT[CG]GGGTT[CG]ATTCTGGGTTTTG[CG
][CG]TCCCCAACCCCCAGGGCTTTC[CG]AAGCATGGCCTGGCT
CCAGGCC[CG]GTCCTGTAAGGACTGGAA[[CG]]GCAGCAAAAT
GTGCAGGGAGGCAGT[CG]GC[CG]GCAGAGCTG[CG]G[CG]GG
AGCCAAGGTCAGGCC[CG][CG]GGGAGAG[[CG]]GGCAGCTTC
CAG[CG]C[CG]GCCACAAGCTCCCAGGCCAGCTGGGC[CG]CAG
ACCCCTTTGCTTCCAGAGAGCACAACC[CG][CG]TCCTTTCTCT
CAGCCAGGCTGCAGTGGCTGCCC[CG]AGCTT[CG]CTTT[CG]T
TTCCCAAGCTGTTAATAA[CG]ATATGTCCCCAAATC[CG]AGGC
T[CG]TGTTTGCTCCCAGATGCCAAGAA[CG]CAACC[CG]AAAT
CCTTCTCCCAAACCC
SEQ ID NO: 5157
GCAAGGTGTC[CG]AAGGGGCATGGGAATTCCTATAAGAAA[CG]
GTCCCTTCTGGATAGT[CG]GAACATAGAGGAAAA[CG]GGGGTG
GGTTAGGGGAACACTTACTCTTGTGGGGCCCTG[CG]CTGG[[CG
]]AATGTCAAGCCTCCAGGTTCACAAATGGCTGGAGGTGCT[CG]
GGGTTAGCTCC[CG]AGGG[CG]GCAAG[CG]C[CG]AC[CG]GT
CCT[CG]CT[CG]CTAGGCAGG[CG]CCTC[CG]GATTC[CG]TT
CATGCCTCT[CG]GAGCTACAGGGCTTGGTG[CG]GAGA[CG]CA
GGG[CG]GG[CG][CG]CTGGGCTCTGGGTGTC[[CG]]CAACCC
AAAGTGCAGCTGGTGTCTTGA[CG]CTAC[CG]TCTATGCACAAG
C[CG]G[CG]G[CG]G[CG]G[CG]GAGCTTGGAGGAGGCAGGGA
GATTCCAAATAAATCCACTAATTCTGGGCTGAG[CG]CTC[CG]G
[CG]TG[CG]AGCC[CG]CCTGCCTGCCAAATGTAACTGTGAAGT
GATGTGGAAAAGTGAGC[[CG]]GGTGCCTCCCCTGA[CG]GATC
TCATTGGGCAGAG[CG]CAGAGGCAGGTCTACCACTTGGAAATTC
ATGTAAGCAGTCCCCCCTCCC[CG]CCCCCTCCC[CG]CCTTCCC
CCTT[CG]CTCTCCC
SEQ ID NO: 5158
ATCTGAGGGGCAGGAGG[CG][CG]CAAGGTGTC[CG]AAGGGGC
ATGGGAATTCCTATAAGAAA[CG]GTCCCTTCTGGATAGT[CG]G
AACATAGAGGAAAA[CG]GGGGTGGGTTAGGGGAACACTTACTCT
TGTGGGGCCCTG[CG]CTGG[[CG]]AATGTCAAGCCTCCAGGTT
CACAAATGGCTGGAGGTGCT[CG]GGGTTAGCTCC[CG]AGGG[C
G]GCAAG[CG]C[CG]AC[CG]GTCCT[CG]CT[CG]CTAGGCAG
G[CG]CCTC[CG]GATTC[CG]TTCATGCCTCT[CG]GAGCTACA
GGGCTTGGTG[CG]GAGA[CG]CAGGG[CG]GG[[CG]][CG]CT
GGGCTCTGGGTGTC[[CG]]CAACCCAAAGTGCAGCTGGTGTCTT
GA[CG]CTAC[CG]TCTATGCACAAGC[CG]G[CG]G[CG]G[CG
]G[[CG]]GAGCTTGGAGGAGGCAGGGAGATTCCAAATAAATCCA
CTAATTCTGGGCTGAG[CG]CTC[[CG]]G[CG]TG[CG]AGCC[
CG]CCTGCCTGCCAAATGTAACTGTGAAGTGATGTGGAAAAGTGA
GC[[CG]]GGTGCCTCCCCTGA[CG]GATCTCATTGGGCAGAG[C
G]CAGAGGCAGGTCTACCACTTGGAAATTCATGTAAGCAGTCCCC
CCTCCC[CG]CCCCCTCCC
SEQ ID NO: 5159
ACCAGAG[CG]CCCAGCTAAG[CG]AGCTGCCTCCACCCCCACTG
TGT[CG]GG[CG]G[CG]CAAACTG[CG]GG[CG]CAGGCAGAGG
AGC[CG][CG]GCTCTG[CG]C[CG]GG[CG]GGAGC[CG]AAGC
CCAGAGCCT[CG]CCAGGGCCTCCC[CG]C[CG]AG[CG]CACTT
AC[CG]T[CG]CTGAGGCAGAG[CG][CG]GTCAGCA[CG]AGGA
CCAGCA[CG]ACCA[CG]ACCTTGG[CG]TTCATGG[CG][CG]G
G[CG]GG[CG]GG[CG]GG[CG]GG[CG]GA[CG]AG[CG][CG]
GGT[CG]GGGGC[CG]GA[CG]C[CG]AG[CG]GGCAATG[CG]G
CTGA[CG]GAGAGTGAAAGTG[CG]G[CG]GTGGGAGG[CG][CG
][CG]C[CG]GG[CG]CTTTAGAGGGGAGAGCCAGG[CG]GGG[C
G]GGG[CG]GGG[CG][CG][CG]GTGAGGGG[CG]GGCC[CG]G
AGC[CG]AGTGCTGGC[CG]TGGC[CG]GGGTCTCTGA[CG][CG
][CG]ACTCCCT[CG]G[CG]GCACCCCCCACCCATCCCCTT[CG
]CCCCTCCTC[CG]CTCCCTCTGTGCTCC[CG]AGG[CG]CAGTG
[CG]CTC[CG]GCCTTTGACCTTCTCAGGCTC[CG]CTGGGCCC[
CG]CAC[CG]CTGGCC[CG]GTCTGCAGTGAGGTC[CG]CAGCCT
GAG[CG]A[CG]C[CG]GCTGGAGGGC[CG]CTTATTGTC
SEQ ID NO: 5160
CCTGGTGG[CG]C[CG]AGAGAAGAGCTAGGCCTGCCCAGCACAG
AGC[CG]GAGAG[CG]T[CG]GGCCTTC[CG]GAAGGGTAAGTTC
TC[CG]CCAAGGGGTCC[CG]AGGGAGCTGGA[[CG]]TCTGAAT
CTGGACTTGCCCCCAGCTT[CG]GGGTT[CG]ATTCTGGGTTTTG
[CG][CG]TCCCCAACCCCCAGGGCTTTC[CG]AAGCATGGCCTG
GCTCCAGGCC[CG]GTCCTGTAAGGACTGGAA[[CG]]GCAGCAA
AATGTGCAGGGAGGCAGT[CG]GC[CG]GCAGAGCTG[CG]G[CG
]GGAGCCAAGGTCAGGCC[CG][CG]GGGAGAG[[CG]]GGCAGC
TTCCAG[CG]C[CG]GCCACAAGCTCCCAGGCCAGCTGGGC[CG]
CAGACCCCTTTGCTTCCAGAGAGCACAACC[CG][CG]TCCTTTC
TCTCAGCCAGGCTGCAGTGGCTGCCC[CG]AGCTT[CG]CTTT[C
G]TTTCCCAAGCTGTTAATAA[CG]ATATGTCCCCAAATC[CG]A
GGCT[CG]TGTTTGCTCCCAGATGCCAAGAA[CG]CAACC[CG]A
AATCCTTCTCCCAAACCCTAGGT[CG]A[CG]AGATGAGTTCCTA
CTTGACCTCTGAGC[CG]AGGTGGGC[CG]GAAAC[CG]AGGCCT
AGGCCC[CG]C[CG]GG
SEQ ID NO: 5161
[CG]CACCAGAG[CG]CCCAGCTAAG[CG]AGCTGCCTCCACCCC
CACTGTGT[CG]GG[CG]G[CG]CAAACTG[CG]GG[CG]CAGGC
AGAGGAGC[CG][CG]GCTCTG[CG]C[CG]GG[CG]GGAGC[CG
]AAGCCCAGAGCCT[CG]CCAGGGCCTCCC[CG]C[CG]AG[CG]
CACTTAC[CG]T[CG]CTGAGGCAGAG[CG][CG]GTCAGCA[CG
]AGGACCAGCA[CG]ACCA[CG]ACCTTGG[CG]TTCATGG[CG]
[CG]GG[CG]GG[CG]GG[CG]GG[CG]GG[CG]GA[CG]AG[CG
][CG]GGT[CG]GGGGC[CG]GA[CG]C[CG]AG[CG]GGCAATG
[CG]GCTGA[CG]GAGAGTGAAAGTG[[CG]]G[[CG]]GTGGGA
GG[CG][CG][CG]C[CG]GG[CG]CTTTAGAGGGGAGAGCCAGG
[CG]GGG[CG]GGG[CG]GGG[CG][CG][CG]GTGAGGGG[CG]
GGCC[CG]GAGC[CG]AGTGCTGGC[CG]TGGC[CG]GGGTCTCT
GA[CG][CG][CG]ACTCCCT[CG]G[CG]GCACCCCCCACCCAT
CCCCTT[CG]CCCCTCCTC[CG]CTCCCTCTGTGCTCC[CG]AGG
[CG]CAGTG[CG]CTC[CG]GCCTTTGACCTTCTCAGGCTC[CG]
CTGGGCCC[CG]CAC[CG]CTGGCC[CG]GTCTGCAGTGAGGTC[
CG]CAGCCTGAG[CG]A[CG]C[CG]GCTGGAGGGC[CG]CTTAT
T
SEQ ID NO: 5162
AG[CG]AGCTGCCTCCACCCCCACTGTGT[CG]GG[CG]G[CG]C
AAACTG[CG]GG[CG]CAGGCAGAGGAGC[CG][CG]GCTCTG[C
G]C[CG]GG[CG]GGAGC[CG]AAGCCCAGAGCCT[CG]CCAGGG
CCTCCC[CG]C[CG]AG[CG]CACTTAC[CG]T[CG]CTGAGGCA
GAG[CG][CG]GTCAGCA[CG]AGGACCAGCA[CG]ACCA[CG]A
CCTTGG[CG]TTCATGG[CG][CG]GG[CG]GG[CG]GG[CG]GG
[CG]GG[CG]GA[CG]AG[CG][CG]GGT[CG]GGGGC[CG]GA[
CG]C[CG]AG[CG]GGCAATG[CG]GCTGA[CG]GAGAGTGAAAG
TG[[CG]]G[CG]GTGGGAGG[CG][CG][CG]C[CG]GG[CG]C
TTTAGAGGGGAGAGCCAGG[CG]GGG[CG]GGG[CG]GGG[CG][
CG][CG]GTGAGGGG[CG]GGCC[CG]GAGC[CG]AGTGCTGGC[
CG]TGGC[CG]GGGTCTCTGA[CG][CG][CG]ACTCCCT[CG]G
[CG]GCACCCCCCACCCATCCCCTT[CG]CCCCTCCTC[[CG]]C
TCCCTCTGTGCTCC[CG]AGG[[CG]]CAGTG[CG]CTC[CG]GC
CTTTGACCTTCTCAGGCTC[CG]CTGGGCCC[CG]CAC[CG]CTG
GCC[CG]GTCTGCAGTGAGGTC[CG]CAGCCTGAG[CG]A[[CG]
]C[CG]GCTGGAGGGC[CG]CTTATTGTCCCTGTGACAGGGCCCC
A
SEQ ID NO: 5163
CTAAG[CG]AGCTGCCTCCACCCCCACTGTGT[CG]GG[CG]G[C
G]CAAACTG[CG]GG[CG]CAGGCAGAGGAGC[CG][CG]GCTCT
G[CG]C[CG]GG[CG]GGAGC[CG]AAGCCCAGAGCCT[CG]CCA
GGGCCTCCC[CG]C[CG]AG[CG]CACTTAC[CG]T[CG]CTGAG
GCAGAG[CG][CG]GTCAGCA[CG]AGGACCAGCA[CG]ACCA[C
G]ACCTTGG[CG]TTCATGG[CG][CG]GG[CG]GG[CG]GG[CG
]GG[CG]GG[CG]GA[CG]AG[CG][CG]GGT[CG]GGGGC[CG]
GA[CG]C[CG]AG[CG]GGCAATG[CG]GCTGA[CG]GAGAGTGA
AAGTG[CG]G[CG]GTGGGAGG[CG][CG][CG]C[[CG]]GG[C
G]CTTTAGAGGGGAGAGCCAGG[CG]GGG[CG]GGG[CG]GGG[C
G][CG][CG]GTGAGGGG[CG]GGCC[CG]GAGC[CG]AGTGCTG
GC[CG]TGGC[CG]GGGTCTCTGA[CG][CG][CG]ACTCCCT[C
G]G[CG]GCACCCCCCACCCATCCCCTT[CG]CCCCTCCTC[CG]
CTCCCTCTGTGCTCC[CG]AGG[[CG]]CAGTG[CG]CTC[CG]G
CCTTTGACCTTCTCAGGCTC[CG]CTGGGCCC[CG]CAC[CG]CT
GGCC[CG]GTCTGCAGTGAGGTC[CG]CAGCCTGAG[CG]A[[CG
]]C[CG]GCTGGAGGGC[CG]CTTATTGTCCCTGTGACAGGGCC
SEQ ID NO: 5164
GGCTG[CG]CTGCCCTGAAGTGGTTCTCCAAGCAG[[CG]][[CG
]]GAGGGTGG[[CG]]GA[CG]G[CG]GA[CG]GAGCCCAGGGGC
[CG][CG]T[CG]GGTGGGGAAACC[CG]AACT[CG][CG]GAGG
GGAATCCCTCCCCCTC[CG]CCCCAGCCCCCCAGCAGCACC[CG]
[CG]GTGGGG[CG]GGGG[CG]CTCTGCCAGCCC[CG]GGAACAG
CAGAGG[CG]G[CG]GCACTGGCTGGACCCA[CG][CG][CG][C
G]CCTC[CG]GGGCTGAAGAAGGAAGGAGTGAGC[CG]AGC[CG]
AGCACCCCACATCTGGAGGGGACAGCCAGC[CG]TGGGCCC[CG]
CCC[CG]G[[CG]]TC[CG]GAGCAGGAGAACTC[CG]AGCTTCT
TGCCCAGGCAGAGAGAGCAGGAG[CG]GAC[CG][CG][CG]CC[
[CG]]GGATTGAGAGTCCTTG[CG]CTCCAGACCCCCACCCAGTG
GC[CG]CCAGGGTCCC[CG]CCTGTC[CG]GACCCT[CG]C[CG]
[CG]CCCAGGCAGG[CG][CG]CCAGGG[CG]GGGCTGACCTGCC
[CG][CG]AAGTTG[CG]GACAGTG[CG]TGAGAAACCAGCACCC
CCTTC[CG]C[CG]CCTCCAGCTTATGGTGAGTGTGGCTGGGGGT
GCAGAGAG[[CG]]CA[CG]GGAATT[CG]GGGGTCTGGGGC[CG
]AGAA[CG]TGA
SEQ ID NO: 5165
CACCACAGGAACCTGACT[[CG]]CAGGAGT[CG]TCCTC[[CG]
]TCTCTA[CG][CG]AAG[CG]CCCCCT[CG]AGGTGGGCAGGGG
GTGG[CG]G[CG]G[CG]A[CG]GTGG[CG]GAGC[CG]CAGAG[
CG]AGCTAGAGAG[CG]AGAG[CG]GTGAGACTCTG[CG]GA[CG
]TCTTCC[CG]CC[CG]C[CG][CG]CTC[CG]CCCCACCCAGCC
TC[CG]CAGCCT[CG]GGC[CG]GAT[CG]CC[CG]G[CG]GGGA
AGATG[CG]CCTCAAGAACCAGGTAGAAG[CG]CCT[CG]G[[CG
]]CAGGCCC[CG]CACCCC[CG]ACTCTGCC[CG]CCCTCTGTTA
[CG]GACACC[CG]CTGGGCCA[[CG]]TGGT[CG][CG]ACTGG
CTTCTCCCAG[CG]GCCAGCCTGGCCACCC[CG]ACTCCCAGGGA
GGGGGAGAGGGCCCTTGCAAGAGGATCCTGCCCC[CG][CG]CTG
CCCAGACCTGAGGGCTCCTGG[CG]ACCCCACC[CG]CAGCCCAT
CTGG[CG]GCTCTGGCCC[CG]CAGGTCAGGGTCACACCCCTGC[
CG]GCCTCCTGAGACTTCC[CG]GCCCCAGGGGAGAGAGCTGAAA
CTTGGGTTGA[CG][CG][CG]CCCCCTCCCCAG[CG]ACAGG[C
G]ACCC[CG]CAGCAGCCC[CG][CG]CCACTCCTCTCTG[CG]C
CTTTC[CG]T
SEQ ID NO: 5166
[CG]TA[CG]TA[CG]TTATCACCCA[CG]GATAT[CG][CG]AA
AGACACAGCTTAGCCCAGAGCCTATGCTAAGAACCCTCATCCACC
ATCTGAGGGGCAGGAGG[CG][CG]CAAGGTGTC[CG]AAGGGGC
ATGGGAATTCCTATAAGAAA[CG]GTCCCTTCTGGATAGT[CG]G
AACATAGAGGAAAA[CG]GGGGTGGGTTAGGGGAACACTTACTCT
TGTGGGGCCCTG[CG]CTGG[[CG]]AATGTCAAGCCTCCAGGTT
CACAAATGGCTGGAGGTGCT[CG]GGGTTAGCTCC[CG]AGGG[C
G]GCAAG[CG]C[CG]AC[CG]GTCCT[[CG]]CT[CG]CTAGGC
AGG[CG]CCTC[CG]GATTC[CG]TTCATGCCTCT[CG]GAGCTA
CAGGGCTTGGTG[CG]GAGA[CG]CAGGG[CG]GG[[CG]][CG]
CTGGGCTCTGGGTGTC[[CG]]CAACCCAAAGTGCAGCTGGTGTC
TTGA[CG]CTAC[[CG]]TCTATGCACAAGC[CG]G[CG]G[CG]
G[CG]G[[CG]]GAGCTTGGAGGAGGCAGGGAGATTCCAAATAAA
TCCACTAATTCTGGGCTGAG[CG]CTC[[CG]]G[CG]TG[CG]A
GCC[CG]CCTGCCTGCCAAATGTAACTGTGAAGTGATGTGGAAAA
GTGAGC[CG]GGTGCCTCCCCTGA[CG]GAT
SEQ ID NO: 5167
TCTCCTTCAACATTTTGAGCATACTGGAATGCCAACCCCCTAGGT
AGGTGCCAGGGCCACCACAGGAACCTGACT[CG]CAGGAGT[[CG
]]TCCTC[CG]TCTCTA[CG][CG]AAG[CG]CCCCCT[CG]AGG
TGGGCAGGGGGTGG[CG]G[CG]G[CG]A[CG]GTGG[CG]GAGC
[CG]CAGAG[CG]AGCTAGAGAG[CG]AGAG[[CG]]GTGAGACT
CTG[CG]GA[[CG]]TCTTCC[CG]CC[CG]C[CG][CG]CTC[C
G]CCCCACCCAGCCTC[CG]CAGCCT[CG]GGC[CG]GAT[CG]C
C[CG]G[CG]GGGAAGATG[CG]CCTCAAGAACCAGGTAGAAG[C
G]CCT[CG]G[CG]CAGGCCC[CG]CACCCC[CG]ACTCTGCC[C
G]CCCTCTGTTA[CG]GACACC[CG]CTGGGCCA[[CG]]TGGT[
CG][CG]ACTGGCTTCTCCCAG[CG]GCCAGCCTGGCCACCC[CG
]ACTCCCAGGGAGGGGGAGAGGGCCCTTGCAAGAGGATCCTGCCC
C[CG][CG]CTGCCCAGACCTGAGGGCTCCTGG[CG]ACCCCACC
[CG]CAGCCCATCTGG[CG]GCTCTGGCCC[CG]CAGGTCAGGGT
CACACCCCTGC[CG]GCCTCCTGAGACTTCC[CG]GCCCCAGGGG
AGAGAGCTGAAACTTGGGTTGA[CG][CG][CG]CCCCCTC
SEQ ID NO: 5168
CCCTTTTTT[CG]TAGCTGCTGA[CG][CG]CTGGTGGTGCCTAT
TAATCATTTACCAGTCCAGAGC[CG][CG]CCAGTTAATGGCTGT
GC[CG]TG[CG]GTGCTCCCACATCCTGGCCTCTCCTCTCCA[CG
]GT[CG]CCTGTGCC[CG]GGCACCC[CG]GAGCTGCAAACTGCA
GAGCCCAGGCAAC[CG]CTGGGCTGTG[[CG]]CCC[CG]C[CG]
G[CG]C[CG]GTAGGAGC[CG][CG]CTCCC[CG]CAG[CG]GTT
G[CG]CTCTACC[CG]GAGG[CG]CTGGG[CG]GCTGTGGGCTGC
AGGCAAG[CG]GT[CG]GGTGGGGAGGGAGGG[CG]CAGG[CG]G
[[CG]]GGTG[CG][CG]AGGAGAAAGCCCCAGCCCTGGCAGCCC
CACTGGCCCCCCTCAGCTGGGATGTTCCCCAATGGCAC[CG]CCT
CCTCTCCTTCCTCCTCTCCTAGCCCCAGCC[CG]GGCAGCTG[CG
]G[CG]AAGG[CG]G[CG]GCAGCAGGGGCCC[CG]GGGC[CG]G
[CG]CTG[CG]GA[CG]GCATGGAGGAGCCAGGG[CG]AAATG[C
G]TCCCAGAA[CG]GGACCTTGAG[CG]AGGGCCAGGGCAG[CG]
CCATCCTGATCTCTTTCATCTACTC[CG]TGGTGTGCCTGGTGGG
GCTGTGTGGGAACTCTATGGTCATCTA
SEQ ID NO: 5169
GAGGGAACCC[CG]GAAGGAGCCACTAAAGTTGAGCAGAGTGCAG
GACTGGGAAGGAAAAAAATGGGGGG[CG]GGGGGGACAAGCCACC
TATGTGCTCTCCTCCCTTTCTCCACCCCACCCCCTTTTTT[CG]T
AGCTGCTGA[CG][CG]CTGGTGGTGCCTATTAATCATTTACCAG
TCCAGAGC[CG][CG]CCAGTTAATGGCTGTGC[CG]TG[CG]GT
GCTCCCACATCCTGGCCTCTCCTCTCCA[CG]GT[CG]CCTGTGC
C[CG]GGCACCC[CG]GAGCTGCAAACTGCAGAGCCCAGGCAAC[
CG]CTGGGCTGTG[CG]CCC[CG]C[CG]G[CG]C[CG]GTAGGA
GC[CG][CG]CTCCC[CG]CAG[CG]GTTG[CG]CTCTACC[CG]
GAGG[CG]CTGGG[CG]GCTGTGGGCTGCAGGCAAG[CG]GT[CG
]GGTGGGGAGGGAGGG[CG]CAGG[CG]G[[CG]]GGTG[CG][[
CG]]AGGAGAAAGCCCCAGCCCTGGCAGCCCCACTGGCCCCCCTC
AGCTGGGATGTTCCCCAATGGCAC[CG]CCTCCTCTCCTTCCTCC
TCTCCTAGCCCCAGCC[CG]GGCAGCTG[CG]G[CG]AAGG[CG]
G[CG]GCAGCAGGGGCCC[CG]GGGC[CG]G[CG]CTG[CG]GA[
CG]GCATGGAGGAGCCAGG
SEQ ID NO: 5170
[CG]TCC[CG]CACATCCTCATAGACAA[CG]CCCTGGGGCTCCT
TCTTTTCTCCAGGG[CG]CCACATCTTCCT[CG]GGCTG[CG]CT
TCATTGACCCCACCC[CG]GA[CG]CCATCTGGTGCATCTGCTCC
CCA[CG]AG[CG][CG]GGCACCTGGGG[CG]AGAGAG[CG]CTG
TCAGC[CG]GCCAGG[CG]CTGGGGTC[CG][CG][CG]AGCC[C
G]AGCTCTCCCT[CG]CTGCAGCTCACAGGCAG[CG]AAAATCT[
CG]CTC[CG][CG]AGAGCCCTGGAGAACTTCTGGAAGGAGTTCT
CAGGCC[CG][CG]CTACTTACAGTGCAGGAAGCAAG[[CG]]GG
G[[CG]]TGAAGAGGAGGTAGAAACACAGACCTCCCTAGCAAAGA
GGGAGG[CG]G[CG]CAG[CG]TCTGC[CG]AGGACT[CG]CCCC
TTCAGGAACAAAGTGACTCCCC[CG]TCC[CG]CTGTCCTGCAGC
TGCAGCAG[CG]CACTCCCAGAGTCAAG[CG]TGTCAGGT[CG]G
TGTGGATGCAG[CG]AATTTGTTGTAATCTACACATTAAGAGCCC
CCTTGCCT[CG]CCCTGCCCCCTCCAAC[CG]TGTGTGTGTGTGT
GTGTGTGTGTGTGTGTGTGTGTGTGTGTGTGTGTGTGTAAAGAGA
CAGGA[CG]AGAG
SEQ ID NO: 5171
[CG]CACATCCTCATAGACAA[CG]CCCTGGGGCTCCTTCTTTTC
TCCAGGG[CG]CCACATCTTCCT[CG]GGCTG[CG]CTTCATTGA
CCCCACCC[CG]GA[CG]CCATCTGGTGCATCTGCTCCCCA[CG]
AG[CG][CG]GGCACCTGGGG[CG]AGAGAG[CG]CTGTCAGC[C
G]GCCAGG[CG]CTGGGGTC[CG][CG][CG]AGCC[CG]AGCTC
TCCCT[CG]CTGCAGCTCACAGGCAG[CG]AAAATCT[CG]CTC[
CG][CG]AGAGCCCTGGAGAACTTCTGGAAGGAGTTCTCAGGCC[
CG][CG]CTACTTACAGTGCAGGAAGCAAG[[CG]]GGG[[CG]]
TGAAGAGGAGGTAGAAACACAGACCTCCCTAGCAAAGAGGGAGG[
CG]G[CG]CAG[CG]TCTGC[CG]AGGACT[CG]CCCCTTCAGGA
ACAAAGTGACTCCCC[CG]TCC[CG]CTGTCCTGCAGCTGCAGCA
G[CG]CACTCCCAGAGTCAAG[CG]TGTCAGGT[CG]GTGTGGAT
GCAG[CG]AATTTGTTGTAATCTACACATTAAGAGCCCCCTTGCC
T[CG]CCCTGCCCCCTCCAAC[CG]TGTGTGTGTGTGTGTGTGTG
TGTGTGTGTGTGTGTGTGTGTGTGTGTGTGTAAAGAGACAGGA[C
G]AGAGAATGA
SEQ ID NO: 5172
T[CG]TAGCTGCTGA[CG][CG]CTGGTGGTGCCTATTAATCATT
TACCAGTCCAGAGC[CG][CG]CCAGTTAATGGCTGTGC[CG]TG
[CG]GTGCTCCCACATCCTGGCCTCTCCTCTCCA[CG]GT[CG]C
CTGTGCC[CG]GGCACCC[CG]GAGCTGCAAACTGCAGAGCCCAG
GCAAC[CG]CTGGGCTGTG[CG]CCC[CG]C[CG]G[CG]C[CG]
GTAGGAGC[CG][CG]CTCCC[CG]CAG[CG]GTTG[CG]CTCTA
CC[CG]GAGG[CG]CTGGG[CG]GCTGTGGGCTGCAGGCAAG[CG
]GT[CG]GGTGGGGAGGGAGGG[CG]CAGG[CG]G[[CG]]GGTG
[CG][[CG]]AGGAGAAAGCCCCAGCCCTGGCAGCCCCACTGGCC
CCCCTCAGCTGGGATGTTCCCCAATGGCAC[CG]CCTCCTCTCCT
TCCTCCTCTCCTAGCCCCAGCC[CG]GGCAGCTG[CG]G[CG]AA
GG[CG]G[CG]GCAGCAGGGGCCC[CG]GGGC[CG]G[CG]CTG[
CG]GA[CG]GCATGGAGGAGCCAGGG[CG]AAATG[CG]TCCCAG
AA[CG]GGACCTTGAG[CG]AGGGCCAGGGCAG[CG]CCATCCTG
ATCTCTTTCATCTACTC[CG]TGGTGTGCCTGGTGGGGCTGTGTG
GGAACTCTATGGTCATCTA[CG]TGATCC
SEQ ID NO: 5173
GCAAAACCTTGAGG[CG]TA[CG]TA[CG]TTATCACCCA[CG]G
ATAT[CG][CG]AAAGACACAGCTTAGCCCAGAGCCTATGCTAAG
AACCCTCATCCACCATCTGAGGGGCAGGAGG[CG][CG]CAAGGT
GTC[CG]AAGGGGCATGGGAATTCCTATAAGAAA[CG]GTCCCTT
CTGGATAGT[CG]GAACATAGAGGAAAA[CG]GGGGTGGGTTAGG
GGAACACTTACTCTTGTGGGGCCCTG[CG]CTGG[[CG]]AATGT
CAAGCCTCCAGGTTCACAAATGGCTGGAGGTGCT[CG]GGGTTAG
CTCC[CG]AGGG[CG]GCAAG[[CG]]C[CG]AC[CG]GTCCT[[
CG]]CT[CG]CTAGGCAGG[CG]CCTC[CG]GATTC[CG]TTCAT
GCCTCT[CG]GAGCTACAGGGCTTGGTG[CG]GAGA[CG]CAGGG
[CG]GG[[CG]][CG]CTGGGCTCTGGGTGTC[[CG]]CAACCCA
AAGTGCAGCTGGTGTCTTGA[CG]CTAC[[CG]]TCTATGCACAA
GC[CG]G[CG]G[CG]G[CG]G[CG]GAGCTTGGAGGAGGCAGGG
AGATTCCAAATAAATCCACTAATTCTGGGCTGAG[CG]CTC[[CG
]]G[CG]TG[CG]AGCC[CG]CCTGCCTGCCAAATGTAACTGTGA
AGTGATGTGGAAAAGTGAGC[CG]GGTGC
SEQ ID NO: 5174
ACCTCCCAGC[CG]TCTC[CG]C[CG]CC[CG]AGCATCCTTGAG
GTGGGA[CG]AGCAGGGGCTTGGATCCCTGC[CG]GC[CG]TCTG
GTGTGTGAGGCTTGCA[CG]GCCCCTGGCTGCCC[CG][CG]CCT
[CG]C[CG]GAGCC[CG]AGGGGG[CG]CAGGTC[CG]GGG[CG]
AGGGC[CG]GC[[CG]]GGCTGTTTGATGGCTTCACTGAGAAGAG
TCAAAGTGCTGTTGGTGTTGAACTTGAT[CG][CG]GTAGC[CG]
GCTT[CG]TGCTCTTCCTGGCCAAGTGC[CG]GCCCAT[CG][CG
]GTG[CG]CAG[CG]GAGA[CG]CCTTCCA[CG]AGATC[CG]GC
[CG][CG][CG]C[CG]AGGTGGCCAACCTCAG[CG][CG]CACA
G[CG]CCAGCCCCATCCAGGATG[CG]GTCCTGAAG[CG]CCTGT
[CG]CTGCTGGAGGACAT[CG]TGTAC[CG]GCAGCTGAATGGTA
AGGA[CG]CA[CG]C[CG]G[CG]CCTC[CG]GGGCT[CG]A[CG
][CG]GG[CG]GGC[CG]GGCACAGGGTGAGCCCCAGGGTCCTTG
[CG][CG]CTG[CG]CCCTGTGCCTGGGAGC[CG]GCACACCCTG
GCTCC[CG][CG][CG]CTCCTTCTTACCCTTCCTGCCC[CG]TT
T[CG]GGCAGATGGC[CG]GGGCTGCTGGAAAGAACAACTTGCCT
GATTCT
SEQ ID NO: 5175
[CG]C[CG]CACTCACAC[CG]GGTGCCTGGGAAAAT[CG]CAGA
[CG]C[CG]GGGAGGAGCAGGGGG[CG]GTGATGGGAAGGGGAGC
TG[CG]AGG[CG]AAGTGTTCTTCAGGGAAG[CG]GGCT[CG]AG
TCTC[CG]CAGCTG[CG]G[CG]G[CG]G[CG]G[CG]G[CG][C
G]CTGGGC[CG]G[CG]G[CG]GG[CG][CG]GGCAGGGGGC[CG
]GGGGTGC[CG][CG][CG]GTAGGAGCCTGGCTTGGACAGC[[C
G]]AGGAGCAGGAAGTGGCCTCTGACCCAGGACAC[CG]AGCAGG
GCTCTCTTGCCTGGATT[CG]CCTTTACAAAGTAACT[CG][CG]
AAGGTCCC[CG]GAGGGG[CG]TCAAG[CG]G[CG]ACAG[CG]G
CC[CG]CC[CG]CC[CG]CAGTC[CG]AAGCCCCCAGCCCTGCTG
TCTTGCCTTCCCCCATTCCATCAGTTGCCATTGCAA[CG]CCAAT
CCTGTTACA[CG]ATACAGACT[CG]CAT[CG]C[CG]AA[CG]G
GGAGAGGAGGAAGAGGAGGAGGAGGAAGAGGAGGAGGAGGAAGAG
GAGGAGTGGGGCTAGGGGAA[CG]GGGGTGGGGAAGAGGGAAGGG
AGGAGGGGGAAGAAGGAGGAGGAAAGGGTCCAACAGGGAG[CG]G
AGTGTG[CG]TCTGGAGCT[CG]CTTGGGCA
SEQ ID NO: 5176
ACACACACTCACACACTCTCCCACAACACAATACCCTGACACACA
CTCACA[CG]CA[CG]C[CG]CACTCACAC[CG]GGTGCCTGGGA
AAAT[CG]CAGA[CG]C[CG]GGGAGGAGCAGGGGG[CG]GTGAT
GGGAAGGGGAGCTG[CG]AGG[CG]AAGTGTTCTTCAGGGAAG[C
G]GGCT[CG]AGTCTC[CG]CAGCTG[CG]G[[CG]]G[CG]G[C
G]G[CG]G[CG][CG]CTGGGC[CG]G[CG]G[CG]GG[CG][CG
]GGCAGGGGGC[CG]GGGGTGC[CG][CG][CG]GTAGGAGCCTG
GCTTGGACAGC[[CG]]AGGAGCAGGAAGTGGCCTCTGACCCAGG
ACAC[[CG]]AGCAGGGCTCTCTTGCCTGGATT[[CG]]CCTTTA
CAAAGTAACT[CG][CG]AAGGTCCC[CG]GAGGGG[CG]TCAAG
[CG]G[CG]ACAG[CG]GCC[CG]CC[CG]CC[CG]CAGTC[CG]
AAGCCCCCAGCCCTGCTGTCTTGCCTTCCCCCATTCCATCAGTTG
CCATTGCAA[CG]CCAATCCTGTTACA[CG]ATACAGACT[CG]C
AT[CG]C[CG]AA[CG]GGGAGAGGAGGAAGAGGAGGAGGAGGAA
GAGGAGGAGGAGGAAGAGGAGGAGTGGGGCTAGGGGAA[CG]GGG
GTGGGGAAGAGGGAAGGGAGGAGGGGGAAGAAG
SEQ ID NO: 5177
ACACAATACCCTGACACACACTCACA[CG]CA[CG]C[CG]CACT
CACAC[CG]GGTGCCTGGGAAAAT[CG]CAGA[CG]C[CG]GGGA
GGAGCAGGGGG[CG]GTGATGGGAAGGGGAGCTG[CG]AGG[CG]
AAGTGTTCTTCAGGGAAG[CG]GGCT[CG]AGTCTC[CG]CAGCT
G[CG]G[[CG]]G[CG]G[CG]G[CG]G[CG][CG]CTGGGC[CG
]G[CG]G[CG]GG[CG][CG]GGCAGGGGGC[CG]GGGGTGC[CG
][CG][CG]GTAGGAGCCTGGCTTGGACAGC[[CG]]AGGAGCAG
GAAGTGGCCTCTGACCCAGGACAC[[CG]]AGCAGGGCTCTCTTG
CCTGGATT[[CG]]CCTTTACAAAGTAACT[CG][CG]AAGGTCC
C[[CG]]GAGGGG[CG]TCAAG[CG]G[CG]ACAG[CG]GCC[CG
]CC[CG]CC[CG]CAGTC[CG]AAGCCCCCAGCCCTGCTGTCTTG
CCTTCCCCCATTCCATCAGTTGCCATTGCAA[CG]CCAATCCTGT
TACA[CG]ATACAGACT[CG]CAT[CG]C[CG]AA[CG]GGGAGA
GGAGGAAGAGGAGGAGGAGGAAGAGGAGGAGGAGGAAGAGGAGGA
GTGGGGCTAGGGGAA[CG]GGGGTGGGGAAGAGGGAAGGGAGGAG
GGGGAAGAAGGAGGAGGAAAGGGTCCAACAGGGAG
SEQ ID NO: 5178
GAGACTC[CG]AGA[CG]CCTAACCCTGGGC[CG][CG]AGGT[C
G]CCTGTTACAAAGGGACAACTTTCTACC[CG]CTC[CG][CG]T
TCCCTCC[CG]ATTCTCCAGCTCTGCCTGGCT[CG]GAATCCCAG
AGCCAGGATGGGAACT[CG]GGGTTGCCT[CG]CCTCCTAGATCT
C[CG]G[CG]AGAGGTC[CG]AGGGGGTGG[CG]GAGAGCTGCAG
GAG[CG]ATGGAGGAGTGGGCAGATTGCTCAAGGGATGGGGGTGC
CCAAAAGCAACAGCCTGCCAAAAACTAAGAGGGA[CG]GGGAGGG
GGGACCTTTGCAGACTTTCTT[CG]TTTTCTTAGATTTCAAACTT
GCAAGGAT[CG]CAAGGATCCAGGGCCCCAGGAAAGGAGGGGTGT
GAAGGACTCAAAATTCCAGCAGCTTGGCTGGGGTGGCTG[CG]CC
AGA[CG]GGCCCTTCCCCAAAGTGCAAACCCACCCCTGTCCT[CG
]GCCC[CG]G[CG][CG]CTCCCTCCCTCAGCC[CG]GGGC[CG]
TACACCACCTGCCCTCTAC[CG]AGAGATCTGGG[CG]G[CG]G[
CG]GC[CG]AAAGCAG[CG]A[CG][CG]CC[CG]GAGCATCCCT
TG[CG]ATA[CG]CTAGGGGA[CG]GTGCTTTCTCTGTCCCAGTT
G[[CG]]TG
SEQ ID NO: 5179
CC[CG]CACTCCACACACTCATGCACACACACACTCACACACACT
CACACACTCTCCCACAACACAATACCCTGACACACACTCACA[CG
]CA[CG]C[CG]CACTCACAC[CG]GGTGCCTGGGAAAAT[CG]C
AGA[CG]C[CG]GGGAGGAGCAGGGGG[CG]GTGATGGGAAGGGG
AGCTG[CG]AGG[CG]AAGTGTTCTTCAGGGAAG[CG]GGCT[CG
]AGTCTC[[CG]]CAGCTG[CG]G[CG]G[CG]G[CG]G[CG]G[
CG][CG]CTGGGC[CG]G[CG]G[CG]GG[CG][CG]GGCAGGGG
GC[CG]GGGGTGC[CG][CG][CG]GTAGGAGCCTGGCTTGGACA
GC[[CG]]AGGAGCAGGAAGTGGCCTCTGACCCAGGACAC[[CG]
]AGCAGGGCTCTCTTGCCTGGATT[CG]CCTTTACAAAGTAACT[
CG][CG]AAGGTCCC[[CG]]GAGGGG[CG]TCAAG[CG]G[CG]
ACAG[CG]GCC[CG]CC[CG]CC[CG]CAGTC[CG]AAGCCCCCA
GCCCTGCTGTCTTGCCTTCCCCCATTCCATCAGTTGCCATTGCAA
[CG]CCAATCCTGTTACA[CG]ATACAGACT[CG]CAT[CG]C[C
G]AA[CG]GGGAGAGGAGGAAGAGGAGGAGGAGGAAGAGGAGGAG
GAGGAAGAGGAGGAGTGGGGCTAGGGGAA[CG]GG
SEQ ID NO: 5180
CCCTTGG[CG]G[CG]GGGG[CG][CG][CG]GGGTGG[CG]GGG
GAGGC[CG]AGG[CG]C[CG]GCAGCTT[CG][CG]C[CG]G[CG
]GCTGGAAG[CG]GG[CG]GGCTGCA[CG]GG[CG]GCT[CG]AG
TG[CG]GGGACCCCAGCCCCT[CG]CCCT[CG]TGAG[CG]C[CG
]CCCCTGCCACCTGCTGCCAAGTCAC[CG]GACTGGGGATG[CG]
GGGGG[CG]GGGTG[CG]GGGAGAGGAAAG[CG]C[CG][CG]GG
TGGCA[CG]GTGCCTCTGG[CG]ACAGAGTAGATGGACC[CG]AA
AGAGACC[[CG]]AGGTGACTT[CG]GGCTGGGCAGGACACCCC[
CG]GGAGTTGCCAG[CG]GCTCTCAG[CG]GG[CG]CCTCTAGGA
CCCCTTGTCACTG[CG]AGCAG[CG]CCTGCTCTGGGGAGGGGCT
[CG]AAGG[CG]C[CG][CG]CCTTCTCAA[CG][CG]C[CG]CT
G[[CG]]TCCCCCAGGGGCTCCAACTTGGACAGCTAGACTGGAAA
TACTGCTGCACAAAGTTAGGCTGAGTT[CG]AAGGACACCAGAGA
GGAAG[CG]CAAGGAGATGCCCTTCTC[CG]GAGC[CG]GGCAGA
G[CG]CCAGAACTCCTAGA[CG]GGAAACTTTCCCCCATTGAAGC
TAGAAC[CG]GTCATG[CG]ACT[CG]GCTAAGTGTGGGGAAACA
SEQ ID NO: 5181
GGCCTCAGTTTCCCTTTCTGTAAAATGCAGGTGAAGAGGCATGAG
CTCAGGATTCCTAAAGCCCTA[CG]GGGCTGAGCAAGGA[CG]GT
GCTTTG[CG]TTTGCCTCCCTTCCCCCAGCC[CG]CC[CG]AGAG
GGTTTGCTGGGGCAGGGTGGCCCCTGGGA[CG]GGTTCCAGACAG
TTCTAGG[[CG]]CTCAGGTC[CG]CAGCTGTGCTACCCC[CG]C
[CG]CC[CG]GGA[CG]CC[CG]GTCCC[CG]CCAGGGCCCTCTC
ACTGTAGCTTTAAGGAGGGCTGGGGC[CG]GGTGCAG[CG]GCAC
CTGGAGTCT[CG]GTGGAACC[[CG]]GCAG[CG]GTGGG[CG][
CG]GACCC[CG]GAGAGGCTGCCC[CG]AGCC[CG]AGCACCCCC
TGAC[CG]GG[CG]CTCACCA[CG]GGTGTTGGGACAAGCAG[CG
]GAGCCTG[CG]GGCTGGAGCCAGGCT[[CG]]GACCCTC[CG][
CG]C[CG]GGG[CG]GG[CG]GC[CG]AG[CG]GA[CG]CAGCTG
GCAGTGG[CG]G[CG]GGAGAG[CG]CCC[CG]GATC[CG]CC[C
G]GAGCCCAACCC[CG]TGGCACTT[CG]GCTGGCTGGGCAG[CG
]G[CG]CTGGGGACAA[CG][CG]GGGCCTTCTGCAA[CG]AG[C
G]CCTG[CG]TGGGACTCCAGGGGC[CG]G[CG]CCC[CG]CT
SEQ ID NO: 5182
AAGGAG[CG]TGAGGAGTGTG[CG]GAGCTTGGCACACCAAGTTG
CTGAGACTGTGACACCAGCCTC[CG]CC[CG]TTC[CG][CG][C
G]GGTGCAGG[CG]G[CG]AGCCT[CG]GCT[CG]GACACCACAG
CTCTCCTGCC[CG][CG]C[CG]GGCAGTCCTCTGCCTGTCCAGA
GGCAGCACTCC[CG]GCTCT[CG]GCTGGCTGTGGGTCTGTCTTG
TGGGGCTCCAGCACTAGCCTGCT[CG]GCCT[CG]GAAACTCCTG
CAG[CG]TCCAGAACACAGAAAATAGACTCATCTCCTAATT[CG]
CCAGGGAGCT[CG]AGGGCTG[CG]GGGC[[CG]][CG]GGGCTG
CCTCCCC[CG]CTCCTCCCCCAACC[CG]ACCCCACCCCACCCC[
CG]CCAGGGCTT[CG]G[CG]GCCTCC[CG]GAGTCACACAGCCT
ACCCCCCCACCCCAACACCCCCTCCCC[CG]GCAGACAAAGGGCC
TGGGCAAATT[CG]C[CG]CC[CG]GCCTCCTAG[CG]CTC[CG]
GGGAGGC[CG]CTG[CG]CCC[CG]GAGTGGAT[CG][CG]CTGG
AGG[CG]TG[CG]C[CG]GG[CG]AGAAGC[CG][CG]GC[CG][
CG]GGAG[CG]CAGTATGGGGAAGAAC[[CG]][[CG]]AGATG[
CG]CCTGACTCACATCTGCTGCTGCTGCCTCCTTTAC
SEQ ID NO: 5183
GACAGAGGAG[CG]A[CG][CG]CTCT[CG]CCTGGATTCTTTTC
CTTTTCTGCCCAGG[CG]AGACCCACTTATTTCCTGGGGT[CG][
CG]TTGGAGAGGGCTTTCA[CG]CC[CG]GGGTG[CG]GAAAAGA
AGCCAAATCCACCTTCCTCCACTGTTGGCCACCCTCCTGCAGGCT
CCCCCAG[CG]C[CG]ACTTCATTTTCTCTCTTCTCT[CG]CTAC
TTCCTCTCTGCCTTTCTTT[CG]TTATTTTTCCAC[CG]AGT[CG
]CCAC[CG]C[CG]CTTGCTTCCT[CG]GAGGAGCAA[CG][CG]
ACTGGCCAGCAAAGGGTGGCTCTGCTC[CG]AG[[CG]]CCAAAG
TTCTCCC[CG]CCTCAGC[CG]G[CG]CCTCCCTGTTGCTGAGCC
CTGG[CG]CCTGCCTGT[CG]CTTGGCTG[CG]CTAAA[CG]CCC
ACTGTTGGTGAGATTCC[CG]AGTGGCACATCC[CG][CG]CCCC
TGGAAAGTCCTCTCCCCTCAAAGGCTGGG[CG]TCATAGTTTGCA
AAATTCCCTAAAACCTAAGAAGGAAAATAGGACTATGAGC[CG]G
TGAGAATTCTGAATTCAGAGT[CG]CACCTGAGGGATCTGAAATT
[CG]GTGGTTCACTGAAATAGGTTCAAAGAGGCTGTGACTTAA[[
CG]]A
SEQ ID NO: 5184
GGGTGAGAGCCTTCAG[CG]GTG[CG]TCCTAAGGAG[CG]TGAG
GAGTGTG[CG]GAGCTTGGCACACCAAGTTGCTGAGACTGTGACA
CCAGCCTC[[CG]]CC[CG]TTC[CG][CG][CG]GGTGCAGG[C
G]G[CG]AGCCT[CG]GCT[CG]GACACCACAGCTCTCCTGCC[C
G][CG]C[CG]GGCAGTCCTCTGCCTGTCCAGAGGCAGCACTCC[
CG]GCTCT[CG]GCTGGCTGTGGGTCTGTCTTGTGGGGCTCCAGC
ACTAGCCTGCT[CG]GCCT[CG]GAAACTCCTGCAG[CG]TCCAG
AACACAGAAAATAGACTCATCTCCTAATT[CG]CCAGGGAGCT[C
G]AGGGCTG[CG]GGGC[[CG]][CG]GGGCTGCCTCCCC[CG]C
TCCTCCCCCAACC[CG]ACCCCACCCCACCCC[CG]CCAGGGCTT
[CG]G[CG]GCCTCC[CG]GAGTCACACAGCCTACCCCCCCACCC
CAACACCCCCTCCCC[CG]GCAGACAAAGGGCCTGGGCAAATT[C
G]C[CG]CC[CG]GCCTCCTAG[CG]CTC[CG]GGGAGGC[CG]C
TG[CG]CCC[CG]GAGTGGAT[CG][CG]CTGGAGG[CG]TG[CG
]C[CG]GG[CG]AGAAGC[CG][CG]GC[CG][CG]GGAG[[CG]
]CAGTATGGGGAAGAAC[[CG]][[CG]]AGATG[CG]CCTGACT
SEQ ID NO: 5185
GTG[CG]GGGACCCCAGCCCCT[CG]CCCT[CG]TGAG[CG]C[C
G]CCCCTGCCACCTGCTGCCAAGTCAC[CG]GACTGGGGATG[CG
]GGGGG[CG]GGGTG[CG]GGGAGAGGAAAG[CG]C[CG][CG]G
GTGGCA[CG]GTGCCTCTGG[CG]ACAGAGTAGATGGACC[CG]A
AAGAGACC[[CG]]AGGTGACTT[CG]GGCTGGGCAGGACACCCC
[CG]GGAGTTGCCAG[CG]GCTCTCAG[CG]GG[CG]CCTCTAGG
ACCCCTTGTCACTG[CG]AGCAG[CG]CCTGCTCTGGGGAGGGGC
T[CG]AAGG[CG]C[CG][CG]CCTTCTCAA[CG][CG]C[CG]C
TG[CG]TCCCCCAGGGGCTCCAACTTGGACAGCTAGACTGGAAAT
ACTGCTGCACAAAGTTAGGCTGAGTT[CG]AAGGACACCAGAGAG
GAAG[[CG]]CAAGGAGATGCCCTTCTC[CG]GAGC[CG]GGCAG
AG[CG]CCAGAACTCCTAGA[CG]GGAAACTTTCCCCCATTGAAG
CTAGAAC[CG]GTCATG[CG]ACT[CG]GCTAAGTGTGGGGAAAC
ACTGGCTCTAGACTCCAGGTGAACAT[CG]CAGGGC[CG]AGGTG
CAGAATACCACCTTGGAGAGAGGGGGTGGCATAGACTGGTGGCAT
TTTGGAGAAGCAT
SEQ ID NO: 5186
[CG]CC[CG]AGAGGGTTTGCTGGGGCAGGGTGGCCCCTGGGA[C
G]GGTTCCAGACAGTTCTAGG[[CG]]CTCAGGTC[CG]CAGCTG
TGCTACCCC[CG]C[CG]CC[CG]GGA[CG]CC[CG]GTCCC[CG
]CCAGGGCCCTCTCACTGTAGCTTTAAGGAGGGCTGGGGC[CG]G
GTGCAG[CG]GCACCTGGAGTCT[CG]GTGGAACC[CG]GCAG[C
G]GTGGG[CG][CG]GACCC[CG]GAGAGGCTGCCC[CG]AGCC[
CG]AGCACCCCCTGAC[CG]GG[CG]CTCACCA[CG]GGTGTTGG
GACAAGCAG[CG]GAGCCTG[CG]GGCTGGAGCCAGGCT[CG]GA
CCCTC[CG][CG]C[CG]GGG[CG]GG[CG]GC[CG]AG[CG]GA
[CG]CAGCTGGCAGTGG[CG]G[CG]GGAGAG[CG]CCC[CG]GA
TC[CG]CC[CG]GAGCCCAACCC[CG]TGGCACTT[CG]GCTGGC
TGGGCAG[CG]G[CG]CTGGGGACAA[CG][CG]GGGCCTTCTGC
AA[CG]AG[CG]CCTG[CG]TGGGACTCCAGGGGC[CG]G[CG]C
CC[CG]CTC[CG]CTCCCCCCAGGCCTCAACCC[CG]CTGGCTAC
C[CG][CG]CTGAAACCTCACCTGC[CG]C[CG]GCTGCAAA[CG
]GACTG[CG]CTCCAGGTGGGTGGAAA[CG]TCCTTCTCCTACTT
TCTGGG
SEQ ID NO: 5187
TTGAACC[CG]GGAGG[CG]GAGGTTGTAGTGAGC[CG]AGATCA
[CG]CCATTACACTCCAGCCTGGG[CG]ACAGAG[CG]AGACTC[
CG]TCTCAAAAAAAAAAAAAAAAAAAAAAAAAAGACTTTCCCCTC
TTTGGGCCTCAGTTTCCCTTTCTGTAAAATGCAGGTGAAGAGGCA
TGAGCTCAGGATTCCTAAAGCCCTA[CG]GGGCTGAGCAAGGA[C
G]GTGCTTTG[CG]TTTGCCTCCCTTCCCCCAGCC[CG]CC[CG]
AGAGGGTTTGCTGGGGCAGGGTGGCCCCTGGGA[CG]GGTTCCAG
ACAGTTCTAGG[[CG]]CTCAGGTC[CG]CAGCTGTGCTACCCC[
CG]C[CG]CC[CG]GGA[CG]CC[CG]GTCCC[CG]CCAGGGCCC
TCTCACTGTAGCTTTAAGGAGGGCTGGGGC[CG]GGTGCAG[CG]
GCACCTGGAGTCT[CG]GTGGAACC[[CG]]GCAG[CG]GTGGG[
CG][CG]GACCC[CG]GAGAGGCTGCCC[CG]AGCC[CG]AGCAC
CCCCTGAC[CG]GG[CG]CTCACCA[CG]GGTGTTGGGACAAGCA
G[CG]GAGCCTG[CG]GGCTGGAGCCAGGCT[[CG]]GACCCTC[
CG][CG]C[CG]GGG[CG]GG[CG]GC[CG]AG[CG]GA[CG]CA
GCTGGCAGTGG[CG]G[CG]GGAGA
SEQ ID NO: 5188
AGTTCTTCTTTGGAT[CG]CCCTTCTCTATTTGCACACTAACATA
GCCAACCTTCCCCAGAGCTGGCCCTC[CG]CCCCCAGCTCCTTGG
CCTGGGTCACACCAGGGTCC[CG]GT[CG]GATTTAGC[CG]GGC
TGCTC[[CG]]GGT[CG]GGTGAGGCACTGGGTG[CG]TGTGGGA
GTCTTCTCTTCTGTCTCTT[CG]AAAGACTAGGGAAATGAACAC[
CG]TTCTGGGCTTGACTTTGTCCCTGCTGGGAATATCCCAGTTCC
TGGCTTTGGAAATCTCAGGCAAATAATTGATTCTTAATGCACACT
TAGGAGA[CG]T[CG]GTAGTA[CG]CT[CG]AGAGCCTCTCTGT
CCCAGAGTGGCTGAGAGCA[CG][CG]CCAGACACAGGTTGGCT[
CG]GCC[CG]AAG[CG]AATTTGGGACTCT[CG]T[CG][CG]GT
GTCCTATTAATTAGCTTTTCC[CG]G[CG]CCTCAGGGACTC[CG
]GGGTGAAGATGGAGAGGTGGCATAATGG[CG]GGACTTGAAGT[
CG]TTGTGGCAGGGACCTGGCAG[CG]TTGTTTTCT[[CG]]GGT
TTTGAAC[CG]GTGTGGAGAAGTAACTTCAGCTGCTGAGTTGGGG
C[CG]CCCTGGCTG[CG][CG]GTGG[CG]GAGGAGGAGAGG
SEQ ID NO: 5189
GAGGAG[CG]A[CG][CG]CTCT[CG]CCTGGATTCTTTTCCTTT
TCTGCCCAGG[CG]AGACCCACTTATTTCCTGGGGT[[CG]][CG
]TTGGAGAGGGCTTTCA[CG]CC[CG]GGGTG[CG]GAAAAGAAG
CCAAATCCACCTTCCTCCACTGTTGGCCACCCTCCTGCAGGCTCC
CCCAG[CG]C[CG]ACTTCATTTTCTCTCTTCTCT[CG]CTACTT
CCTCTCTGCCTTTCTTT[CG]TTATTTTTCCAC[CG]AGT[CG]C
CAC[CG]C[CG]CTTGCTTCCT[CG]GAGGAGCAA[CG][CG]AC
TGGCCAGCAAAGGGTGGCTCTGCTC[CG]AG[CG]CCAAAGTTCT
CCC[CG]CCTCAGC[CG]G[CG]CCTCCCTGTTGCTGAGCCCTGG
[CG]CCTGCCTGT[CG]CTTGGCTG[[CG]]CTAAA[[CG]]CCC
ACTGTTGGTGAGATTCC[CG]AGTGGCACATCC[CG][CG]CCCC
TGGAAAGTCCTCTCCCCTCAAAGGCTGGG[CG]TCATAGTTTGCA
AAATTCCCTAAAACCTAAGAAGGAAAATAGGACTATGAGC[CG]G
TGAGAATTCTGAATTCAGAGT[CG]CACCTGAGGGATCTGAAATT
[CG]GTGGTTCACTGAAATAGGTTCAAAGAGGCTGTGACTTAA[[
CG]]AGCTT
SEQ ID NO: 5190
[CG]GCTCTGCC[CG][CG]GCTGAGCCC[CG]CACAC[CG][CG
]C[CG]CTGTCCTTGGGGTCC[CG]GCACA[CG]G[CG]TTCCCT
CCC[CG]CCCC[CG]CCATCCACTCCCAC[CG]CCTCTCCAGTCC
CTGTGC[CG]TCTCCAC[CG]CCCTCTGACTCC[CG]CAC[CG]A
GCTG[CG]TCTC[CG]C[CG]C[CG]GCCTCTCTCAG[CG][CG]
GCC[CG]GCCTCCTCTTTGTCTTTGAAGCTCCCC[CG]C[CG]AG
GCC[CG][CG][CG][CG]GCTCCCCTCTC[CG]TCCCTCTC[CG
]GCCCTTGGG[CG]CCCAGT[CG]GAGGT[CG]GGG[CG]GGATG
GGGCA[CG]AGTCT[CG]GGG[CG]GCC[CG][CG]CTCCCCCTA
CTAGTCCCCTCATTGCCT[CG]GCCC[CG]TCCC[CG]CAGCCT[
CG]GGG[CG]GGAGGG[CG]CAGGGGTCCC[CG]GG[CG]CCCTG
GGAGCCCATCTCCCTCAGGTC[CG][CG]C[CG]GTGTCAGC[CG
]C[CG]G[CG]GGGGG[CG]GGTGGAGATTAAGATGAGTGCTG[C
G]AGGGA[CG]GGGC[CG]AGGGTTTTGTGTAGAAGGAGAGCAGG
ATTAGTGGAGAGAAAGTGCTGACC[CG]GGGTCAGC[CG]CAAGT
CAAACCCTTTTACACTCAGC[CG]G[CG]GAA[CG]AGCA[CG]G
CT[CG]GGC[CG]G
SEQ ID NO: 5191
[CG][CG][CG]GGGGG[CG]GAGAGGA[CG]TAG[CG]CT[CG]
GCC[CG]G[[CG]]CC[CG]CCCCCTAGTGCTGCCCCCACTTTGA
GAGTAATTGAGACTTGAG[CG]TGACTG[CG]GAGCCTGGAGGAT
G[CG]GGCTCCC[CG]AG[CG]GGCTTCCCCTCTGCC[CG]G[CG
][CG]ATGGAG[CG]GGT[CG]GTGAG[CG]GAACAAAGG[CG]C
CTGCC[CG]GGGGAG[CG]GGTCTTC[CG]CTGAAGAG[CG][CG
]GGTGG[CG][CG]C[CG][CG]A[CG]GC[CG]CCTG[CG]GGC
CAAGGTGGGAGGCTGTG[CG]GCCCCAGG[CG][CG]G[CG][CG
]CTC[CG]C[CG]TGGG[CG]GG[[CG]]G[CG]ATCCCTGAGTC
[CG]GCACCTGTG[CG]C[CG]CTCCCTCTGTGCCTCC[CG]GGC
AGCCC[CG]CCTGC[CG]GCCT[CG]GAGTC[CG][CG]G[CG]C
[CG]G[CG]GCTAGAGGTCCAGAGG[[CG]]AACCACTTGCTGGT
GCAGAAGAGAAACCCTCAAATCCCTGGCCTTT[CG][CG]GAGA[
CG]CCTGGA[CG]GGGC[CG]TG[CG]C[CG]TGGACTGAGCAGG
[CG]TCT[CG]GGGAGCACTTCTGCAGAG[CG]AGGACTTCCATG
TGAG[CG]ATTC[[CG]]TTCTCCCCACCACCAATC[CG]ACCTC
CCAGC[CG]TCTC[CG]C[CG]CC[CG]AGCATCCT
SEQ ID NO: 5192
CTC[CG][CG][CG]GGGGG[CG]GAGAGGA[CG]TAG[CG]CT[
CG]GCC[CG]G[CG]CC[CG]CCCCCTAGTGCTGCCCCCACTTTG
AGAGTAATTGAGACTTGAG[CG]TGACTG[CG]GAGCCTGGAGGA
TG[CG]GGCTCCC[CG]AG[CG]GGCTTCCCCTCTGCC[CG]G[C
G][CG]ATGGAG[CG]GGT[CG]GTGAG[CG]GAACAAAGG[CG]
CCTGCC[CG]GGGGAG[CG]GGTCTTC[CG]CTGAAGAG[CG][C
G]GGTGG[CG][CG]C[CG][CG]A[CG]GC[CG]CCTG[CG]GG
CCAAGGTGGGAGGCTGTG[CG]GCCCCAGG[CG][CG]G[CG][C
G]CTC[CG]C[CG]TGGG[CG]GG[[CG]]G[[CG]]ATCCCTGA
GTC[CG]GCACCTGTG[CG]C[CG]CTCCCTCTGTGCCTCC[CG]
GGCAGCCC[CG]CCTGC[CG]GCCT[CG]GAGTC[CG][CG]G[C
G]C[CG]G[CG]GCTAGAGGTCCAGAGG[[CG]]AACCACTTGCT
GGTGCAGAAGAGAAACCCTCAAATCCCTGGCCTTT[CG][CG]GA
GA[CG]CCTGGA[CG]GGGC[CG]TG[CG]C[CG]TGGACTGAGC
AGG[CG]TCT[CG]GGGAGCACTTCTGCAGAG[CG]AGGACTTCC
ATGTGAG[CG]ATTC[[CG]]TTCTCCCCACCACCAATC[CG]AC
CTCCCAGC[CG]TCTC[CG][CG]CC[CG]AGCAT
SEQ ID NO: 5193
GGTGGCTTTGTC[CG]GGTCC[CG]CTCC[CG]AGTCCCAGGCTC
CTGCTGCCCTCCATCTCTTAGCACTCCC[CG]GGGGCTC[CG]AC
T[CG][CG]CCCTCTCCC[CG]TTCTGCCTTCTCCTGATC[CG]C
TC[CG]CTC[CG]TCTCCTC[CG]GC[CG]GAG[CG]TGCAGCAA
C[CG]CCATGGATGCCCCAGTGCCC[CG]AGCC[CG]CTCCAGGG
ATAG[CG]CT[CG]GTCCC[CG]GGGGCCACTGAGAACAGGGGA[
CG]CACTA[CG]AGGGGCAGGGG[CG]TGGCAGGAGCCCTGCCTG
GC[CG]CCCCTGGTGGGAAAAGCCCCTAGGGGT[[CG]]AGGC[C
G]AG[CG]TGGAG[CG]GAGCTGGGGTG[CG]G[CG]AGGGGCAG
CAGGAGC[CG]C[CG]C[CG]CAGA[CG]CC[CG]GGG[CG]CAG
AGGATCAAGAGCCC[CG][CG]C[CG]G[CG]AGGAG[CG][CG]
CTCAGC[CG]GGCTTGGGAGC[CG]C[CG]C[CG]G[CG]CCAAC
TTTTCCCAT[CG][CG]GGAG[CG]AAGAGCAG[CG]GAGAGAGG
GGTCCCAGAA[CG]AAGGTGG[CG]GCA[CG]AGCTCTG[CG]CT
GG[CG]GCTGTGGGGGGC[[CG]]G[CG]CTCAGGACCCCAACTC
CATCCAAGTTG[[CG]][[CG]][CG]GTGGGGG[CG]GG[CG]G
AGG[CG]G[CG]C[CG]GG
SEQ ID NO: 5194
GGAG[CG]CA[CG]GAGATGCCTAGGGAGGAGGTGA[CG]G[CG]
C[CG]TC[CG]C[CG]A[CG]GGCTGGAGT[CG]CACACCACCAG
GCACTTGCCCTCCAGCA[CG]ATGGGCTC[CG]TGT[CG]TTCTG
[CG]CC[CG]CA[CG]GGGCAGCAGG[CG]GGCAGTAGCAGCAAC
AGCAGGGCCAG[CG]CCACCCCCAGGCAGGATC[CG]CAGC[CG]
CC[CG]GCT[CG][CG]CAG[CG]CCCCC[CG]G[CG]CC[CG]G
GCATCATCAGC[CG]CAGCC[CG]AGTGGCCCC[CG]GC[CG]GG
[CG]CCTGCAT[CG]GGACTGGTGGGAGG[CG]G[CG][CG][CG
]GGGGTGGAGGC[[CG]]G[CG]C[CG]G[CG][CG]AG[CG]G[
CG][CG]GAAGGG[CG][CG]AAGGAA[CG][CG][CG]GAGCT[
CG]CAGCAGCCTC[CG]GGGGCCTT[CG]TCCC[CG]GCTCTGA[
CG]TTCAAGGCCAGGGT[CG]TTCTCAGAAGAAAGG[[CG]]CCT
GTGAACCTGTCAGGGCACAGAACCCAAAGCCTTACAC[CG]GGAG
GTGGAGCCAC[CG]GGAAGAAGGGGGACTGGGAGCAGGTGCCTG[
CG]GCT[CG]GGGGTTCTCCC[CG]AAGGCCACCCCCTTGGTCC[
CG][CG]CACAGCCAACC[CG]CTTTC[CG]ATCTGGCACAACAG
GTC[CG]GAGAAA[CG]C
SEQ ID NO: 5195
A[CG]CC[CG]G[CG]GCCC[CG]GCTT[CG][CG]GCCAC[CG]
G[CG]TTGTCTCCACTCT[CG]C[CG][CG]GGCAGTGGCCAGCC
AGTGGC[CG]GAGGAGCTGG[CG]T[CG]G[CG][CG]GAGAGC[
CG]C[CG]TGCTGGGG[CG]C[CG]GGC[CG]GACCAGAGCTGCT
GCCCCAGCAGGG[CG]G[CG]GCAGAGG[CG]GTGAGATGCAGGT
GGAAGC[CG]GAGGGACATCAC[CG]GCAGG[CG]AG[CG]G[CG
]GGGC[CG]GGGCATCCCAGCTCCTGCCAAGCTTGG[CG]G[CG]
[CG]AGGAGGAGT[[CG]]C[CG]GG[CG]CAGCCCCCAATCACC
CAGGAA[CG][CG]GGGA[[CG]]CCTGGGCCACTGCTC[CG]GC
[CG]ATGGTTCCAGAGGAAGC[CG]TCCCCTTGCTAAGGGTTCC[
CG]GGAGGAGGTGAAGG[CG]C[CG][CG]GGCTGGGGGGT[CG]
G[CG]GCTGAAGACCTC[CG]GCTGCCCAGCACCTCCTT[CG][C
G]CTGAC[CG]GGGACT[CG]GCCCACAACCAAGCCATGGTGCAC
TGGT[CG]GGACACAACAGCAG[CG]TGAGTACCCACC[CG]G[C
G]G[CG]GGTC[CG]CCTGTTTCCTGACAC[CG]AAGGGGAATGG
GGGGGTGGGTGGGAG[CG]AGGGACAGATAGTTGCT[CG]GGGGT
[CG]AGG[CG]G
SEQ ID NO: 5196
GATCTGGATCTCAGCCACCTC[CG]GGGGGACC[CG]GCCCCTGC
CC[[CG]]CACTCCA[CG][CG]AAGGGATGCCAGACCCT[CG]G
CCTCCTGGA[CG]CACAG[CG]GGGGCT[CG]CACCCCAACCC[C
G]GGAACACCCTGGCCCCTTTCCCTAA[CG]CTGCCCACTGAGG[
CG]TCCTCAC[CG]TCTACCCTGTTGGGGT[CG]CTCTCTGACCC
C[CG][CG]CTGCATCCC[CG]GGAC[CG]ACCCACCTGCTG[CG
]CAGCTACCCCAGGC[CG]GTCCCAGC[CG]CC[CG]GAGCCCCA
GTG[CG][CG]ATGG[CG]GC[CG]GCAAACTG[CG]CCTG[CG]
CACTGGGCCTCAC[CG][CG]GACTA[CG]ACTCCCACAATGC[C
G][CG]AGGCTGTGC[CG][CG]CAC[CG]GGG[CG]AGCAAGGG
AGC[CG]TGACAGGTGACAGTG[CG]CATGGCCAATCAGA[CG][
CG]AGTGTGGCCTCC[CG]G[CG]TCC[CG]CCCACTC[CG]G[C
G]CCTG[CG]TCATTTTGGGCACCATGGCAA[CG]AAGTAAACAA
G[CG]GGTAAATAATAGACCCCTGACAGCCAGAGACC[CG]GAGA
AACAAAGA[CG]GTGAGAAA[CG]GAGAAACAAAGACAAAAAGA[
CG][CG]CAGAC[CG]GGAGGGAGAGAAGCATGAAGG
SEQ ID NO: 5197
CTGGC[CG]CCCCTGGTGGGAAAAGCCCCTAGGGGT[[CG]]AGG
C[CG]AG[CG]TGGAG[CG]GAGCTGGGGTG[CG]G[CG]AGGGG
CAGCAGGAGC[CG]C[CG]C[CG]CAGA[CG]CC[CG]GGG[CG]
CAGAGGATCAAGAGCCC[CG][CG]C[CG]G[CG]AGGAG[CG][
CG]CTCAGC[CG]GGCTTGGGAGC[CG]C[CG][CG]G[CG]CCA
ACTTTTCCCAT[CG][CG]GGAG[CG]AAGAGCAG[CG]GAGAGA
GGGGTCCCAGAA[CG]AAGGTGG[CG]GCA[CG]AGCTCTG[CG]
CTGG[CG]GCTGTGGGGGGC[[CG]]G[CG]CTCAGGACCCCAAC
TCCATCCAAGTTG[[CG]]C[CG][CG]GTGGGGG[CG]GG[CG]
GAGG[CG]G[CG]C[CG]GGCAGGTGG[CG]GC[CG]CTTGCC[C
G]GGCTGGGCTTGTGC[CG]AG[CG][CG]GGTC[CG]GA[CG]G
AG[CG]AGC[CG]C[CG]C[CG][CG][CG]GGGGTGGAGCTGAG
CTGAAGTCACTGG[CG]GAG[CG]GG[CG][CG]GG[CG]CTGG[
[CG]]GG[[CG]]A[CG][CG]GGAGGGAGGGCAGG[CG]CTGC[
CG]GGACTC[CG]GG[CG]C[CG][CG]TCC[CG][CG]GC[CG]
AGCTG[CG]CTG[CG]CAC[CG]GGTCTC[CG]GG[CG]CTGGAG
G[CG]C[CG]C[CG]G[CG]ACCTCCCCT[CG]GCCT[CG]CAGG
[CG]CTGC[CG]C[CG]CCTCT
SEQ ID NO: 5198
TGCAGGGCCCCTCAC[CG]ATCTGGATCTCAGCCACCTC[CG]GG
GGGACC[CG]GCCCCTGCCC[[CG]]CACTCCA[CG][CG]AAGG
GATGCCAGACCCT[CG]GCCTCCTGGA[CG]CACAG[CG]GGGGC
T[CG]CACCCCAACCC[CG]GGAACACCCTGGCCCCTTTCCCTAA
[CG]CTGCCCACTGAGG[CG]TCCTCAC[CG]TCTACCCTGTTGG
GGT[CG]CTCTCTGACCCC[CG][CG]CTGCATCCC[CG]GGAC[
CG]ACCCACCTGCTG[CG]CAGCTACCCCAGGC[CG]GTCCCAGC
[CG]CC[CG]GAGCCCCAGTG[CG][CG]ATGG[CG]GC[[CG]]
GCAAACTG[CG]CCTG[CG]CACTGGGCCTCAC[CG][CG]GACT
A[CG]ACTCCCACAATGC[CG][CG]AGGCTGTGC[CG][CG]CA
C[CG]GGG[CG]AGCAAGGGAGC[CG]TGACAGGTGACAGTG[CG
]CATGGCCAATCAGA[CG][CG]AGTGTGGCCTCC[CG]G[CG]T
CC[CG]CCCACTC[CG]G[CG]CCTG[CG]TCATTTTGGGCACCA
TGGCAA[[CG]]AAGTAAACAAG[[CG]]GGTAAATAATAGACCC
CTGACAGCCAGAGACC[CG]GAGAAACAAAGA[CG]GTGAGAAA[
CG]GAGAAACAAAGACAAAAAGA[CG][CG]CAGAC[CG]GGAGG
SEQ ID NO: 5199
TA[CG]AGGGGCAGGGG[CG]TGGCAGGAGCCCTGCCTGGC[CG]
CCCCTGGTGGGAAAAGCCCCTAGGGGT[[CG]]AGGC[CG]AG[C
G]TGGAG[CG]GAGCTGGGGTG[CG]G[CG]AGGGGCAGCAGGAG
C[CG]C[CG]C[CG]CAGA[CG]CC[CG]GGG[CG]CAGAGGATC
AAGAGCCC[CG][CG]C[CG]G[CG]AGGAG[CG][CG]CTCAGC
[CG]GGCTTGGGAGC[CG]C[CG]C[CG]G[CG]CCAACTTTTCC
CAT[CG][CG]GGAG[CG]AAGAGCAG[CG]GAGAGAGGGGTCCC
AGAA[CG]AAGGTGG[CG]GCA[CG]AGCTCTG[CG]CTGG[CG]
GCTGTGGGGGGC[[CG]]G[[CG]]CTCAGGACCCCAACTCCATC
CAAGTTG[CG]C[CG][CG]GTGGGGG[CG]GG[CG]GAGG[CG]
G[CG]C[CG]GGCAGGTGG[CG]GC[CG]CTTGCC[CG]GGCTGG
GCTTGTGC[CG]AG[CG][CG]GGTC[CG]GA[CG]GAG[CG]AG
C[CG]C[CG]C[CG][CG][CG]GGGGTGGAGCTGAGCTGAAGTC
ACTGG[CG]GAG[CG]GG[CG][CG]GG[CG]CTGG[CG]GG[CG
]A[CG][CG]GGAGGGAGGGCAGG[CG]CTGC[CG]GGACTC[CG
]GG[CG]C[CG][CG]TCC[CG][CG]GC[CG]AGCTG[CG]CTG
[CG]CAC[CG]GGTCTC[CG]GG[CG]CTGGAGG[CG]C[CG]C[
CG]G[CG]ACCTC
SEQ ID NO: 5200
[CG][CG]C[CG]GACC[CG]CACCT[CG]G[CG]GG[CG]CCAC
ACACT[CG]GCAGCC[CG]AGC[CG][CG]GTAGC[CG]CAG[CG
]GGATGGAGG[CG]G[CG][CG]CA[CG]GAG[CG]CCC[CG]CA
GGCAGGC[CG]GGGG[CG]C[CG]CTTGTC[CG]GA[CG]GGGCT
CCTACTCTTGT[CG]A[CG]TGGGTCCTGGC[CG]G[CG]C[CG]
AGATCACTTGGGA[CG][CG]ACAGG[CG]GTCC[CG]GA[CG]C
C[CG]G[CG]GCCC[CG]GCTT[CG][CG]GCCAC[CG]G[CG]T
TGTCTCCACTCT[CG]C[CG][CG]GGCAGTGGCCAGCCAGTGGC
[CG]GAGGAGCTGG[CG]T[CG]G[CG][CG]GAGAGC[[CG]]C
[CG]TGCTGGGG[CG]C[CG]GGC[CG]GACCAGAGCTGCTGCCC
CAGCAGGG[CG]G[CG]GCAGAGG[CG]GTGAGATGCAGGTGGAA
GC[CG]GAGGGACATCAC[CG]GCAGG[CG]AG[CG]G[CG]GGG
C[CG]GGGCATCCCAGCTCCTGCCAAGCTTGG[CG]G[CG][CG]
AGGAGGAGT[[CG]]C[CG]GG[CG]CAGCCCCCAATCACCCAGG
AA[CG][CG]GGGA[[CG]]CCTGGGCCACTGCTC[CG]GC[CG]
ATGGTTCCAGAGGAAGC[CG]TCCCCTTGCTAAGGGTTCC[CG]G
GAGGAGGTGAAGG[CG]C[CG][CG]GGCTGGGGGGT[CG]G[CG
]GC
SEQ ID NO: 5201
GG[CG]C[CG]AGATCACTTGGGA[CG][CG]ACAGG[CG]GTCC
[CG]GA[CG]CC[CG]G[CG]GCCC[CG]GCTT[CG][CG]GCCA
C[CG]G[CG]TTGTCTCCACTCT[CG]C[CG][CG]GGCAGTGGC
CAGCCAGTGGC[CG]GAGGAGCTGG[CG]T[CG]G[CG][CG]GA
GAGC[[CG]]C[CG]TGCTGGGG[CG]C[CG]GGC[CG]GACCAG
AGCTGCTGCCCCAGCAGGG[CG]G[CG]GCAGAGG[CG]GTGAGA
TGCAGGTGGAAGC[CG]GAGGGACATCAC[CG]GCAGG[CG]AG[
CG]G[CG]GGGC[CG]GGGCATCCCAGCTCCTGCCAAGCTTGG[C
G]G[CG][CG]AGGAGGAGT[[CG]]C[CG]GG[CG]CAGCCCCC
AATCACCCAGGAA[CG][CG]GGGA[[CG]]CCTGGGCCACTGCT
C[CG]GC[CG]ATGGTTCCAGAGGAAGC[CG]TCCCCTTGCTAAG
GGTTCC[CG]GGAGGAGGTGAAGG[CG]C[CG][CG]GGCTGGGG
GGT[CG]G[CG]GCTGAAGACCTC[CG]GCTGCCCAGCACCTCCT
T[CG][CG]CTGAC[CG]GGGACT[CG]GCCCACAACCAAGCCAT
GGTGCACTGGT[CG]GGACACAACAGCAG[CG]TGAGTACCCACC
[CG]G[CG]G[CG]GGTC[CG]CCTGTTTCCTGACAC[CG]AAGG
GGAATGGGGGGGTGGGTG
SEQ ID NO: 5202
[CG]GGGCAGCAGG[CG]GGCAGTAGCAGCAACAGCAGGGCCAG[
CG]CCACCCCCAGGCAGGATC[CG]CAGC[CG]CC[CG]GCT[CG
][CG]CAG[CG]CCCCC[CG]G[CG]CC[CG]GGCATCATCAGC[
CG]CAGCC[CG]AGTGGCCCC[CG]GC[CG]GG[CG]CCTGCAT[
CG]GGACTGGTGGGAGG[CG]G[CG][CG][CG]GGGGTGGAGGC
[CG]G[CG]C[CG]G[CG][CG]AG[CG]G[CG][CG]GAAGGG[
CG][CG]AAGGAA[CG][CG][CG]GAGCT[CG]CAGCAGCCTC[
CG]GGGGCCTT[CG]TCCC[CG]GCTCTGA[CG]TTCAAGGCCAG
GGT[CG]TTCTCAGAAGAAAGG[CG]CCTGTGAACCTGTCAGGGC
ACAGAACCCAAAGCCTTACAC[CG]GGAGGTGGAGCCAC[CG]GG
AAGAAGGGGGACTGGGAGCAGGTGCCTG[CG]GCT[CG]GGGGTT
CTCCC[CG]AAGGCCACCCCCTTGGTCC[CG][CG]CACAGCCAA
CC[CG]CTTTC[CG]ATCTGGCACAACAGGTC[CG]GAGAAA[CG
]CC[CG]GG[CG]CAGCTGGCAG[CG]CATCCCTGAAGGCTCC[C
G]AGCAG[CG][CG][CG]GGGCTG[CG]A[CG]AGGGAGGGGGC
[CG][CG]CTTCTGCAGGTCCT[CG]T[CG]CTAAGA[CG]GGCC
C[CG]AGACCCTCAGA
SEQ ID NO: 5203
GAGGGGCAGGGG[CG]TGGCAGGAGCCCTGCCTGGC[CG]CCCCT
GGTGGGAAAAGCCCCTAGGGGT[[CG]]AGGC[CG]AG[CG]TGG
AG[CG]GAGCTGGGGTG[CG]G[CG]AGGGGCAGCAGGAGC[CG]
C[CG]C[CG]CAGA[CG]CC[CG]GGG[CG]CAGAGGATCAAGAG
CCC[CG][CG]C[CG]G[CG]AGGAG[CG][CG]CTCAGC[CG]G
GCTTGGGAGC[CG]C[CG]C[CG]G[CG]CCAACTTTTCCCAT[C
G][CG]GGAG[CG]AAGAGCAG[CG]GAGAGAGGGGTCCCAGAA[
CG]AAGGTGG[CG]GCA[CG]AGCTCTG[CG]CTGG[CG]GCTGT
GGGGGGC[[CG]]G[CG]CTCAGGACCCCAACTCCATCCAAGTTG
[CG]C[CG][CG]GTGGGGG[CG]GG[CG]GAGG[CG]G[CG]C[
CG]GGCAGGTGG[CG]GC[CG]CTTGCC[CG]GGCTGGGCTTGTG
C[CG]AG[CG][CG]GGTC[CG]GA[CG]GAG[CG]AGC[CG]C[
CG]C[CG][CG][CG]GGGGTGGAGCTGAGCTGAAGTCACTGG[C
G]GAG[CG]GG[CG][CG]GG[CG]CTGG[CG]GG[CG]A[[CG]
][CG]GGAGGGAGGGCAGG[CG]CTGC[CG]GGACTC[CG]GG[C
G]C[CG][CG]TCC[CG][CG]GC[CG]AGCTG[CG]CTG[CG]C
AC[CG]GGTCTC[CG]GG[CG]CTGGAGG[CG]C[CG]C[CG]G[
CG]ACCTCCCC
SEQ ID NO: 5204
CAAGAGAAAAGGCACAAAAACCCTATTGAGAGGCCC[CG]GC[CG
]CTCCTGG[CG]TAGTCCATCACATGGCAATAGTCCTATTTAAGC
[CG][[CG]][[CG]][CG]G[CG]CCTTTCAGCACCACTAG[CG
]CTGGCCAGCACCC[CG][[CG]]CTCTTTGGG[CG]GTGCCCA[
CG]GCAGCAGAGGCTACTGTTTCAGCCTAGGTCTCAGC[CG][CG
][CG]TTCAGCCTCCTGGGCAGAGGCAGCTG[CG]GGATAC[CG]
[CG]GCCAGGGAAAG[CG][[CG]]TGGAGAGC[CG]AAAGGTG[
CG]GTGGG[CG]CAGAGGG[CG]GGCTGGCTG[CG]GGG[CG]AC
[CG][CG][CG]C[CG]GGGCCATGC[CG][CG]CTCCTTCCTGG
TGGACT[CG]CTAGTGCTG[CG][CG]AGG[CG]GG[CG]AGAAG
AAGG[CG]CC[CG]AGGGCAGCC[CG]C[CG]C[CG]CTCTTCCC
CTA[CG]CTGTGCCCC[CG]C[CG]CA[CG][CG]CTGCA[CG]G
TCTCT[CG]CCTGG[CG]CCTGCCA[CG][CG][CG]CAAGGCTG
GGCTGCTGTG[CG]TGTGCC[CG]CTCTG[CG]TCAC[CG]CCT[
CG]CAGCTGCATGGGCCCCC[CG]GGC[CG]CC[CG][CG]CTGC
CTCTACTCAAGGCTTCCTTCCCACCCTT[CG]GCT[CG]CAGTAC
TGCCA[CG][CG]CCCCTGG
SEQ ID NO: 5205
AACCTGAGGTCAGAGCTGAGG[CG]TAGGGGC[CG]GGGTAGGGG
CAGAGGTGC[CG]AAA[CG]CACTAGCTGGGGACTCC[CG]GGGG
AAGGAGATAGGAGAGACCCAAGG[CG]AGCCTTAGGACT[CG]CC
CCTATGACTATGACTA[CG][CG]CTG[CG]TC[CG]AACCACAT
GTCCACCC[CG]CATTTGCCTT[CG]GGGACCAGAC[CG][CG]C
TCCCTCAGATGGCTGGGGAGGGAGCAGACTC[CG]GGTGAAGGCT
GGGGGAGGTAC[CG]GGATGC[CG]AGTA[CG]CCAGAGCAGG[C
G]GGGGATGGGTTC[CG]GCTTCCTGC[CG]CCT[CG]GCATCT[
CG]CTTTGCAC[CG]GGCAAAGAAGGGGCCA[CG]AC[CG]G[CG
]AAGAG[CG][CG]TGGAGACACAGGGACC[CG][CG]ATCCAGG
GGCAGGAACCC[CG]CCCCCTCC[CG]GAAACCTCC[CG]GGCCC
TGAGT[CG]TGCC[CG]G[CG]CTCCCCACCCCACCCCACCCC[C
G]C[CG]CCCCTGC[CG]TCACTCA[CG]GC[CG]CTG[CG]GGC
TGGCTAG[CG]TGTG[CG]CTCTC[CG][CG][CG]CAGGCACCT
GCT[CG]CTGGG[CG]CTGTG[CG]CTCACTTCTCCCT[CG][CG
]CACTGCAGACTCC[CG]G[CG]CTGC[CG]C[CG]CCAC
SEQ ID NO: 5206
TTACCTGGGATCAGCAGGGAGCC[CG]GG[CG][CG]C[CG][CG
]G[CG]TGGGGACTAGGCT[CG]GG[CG][CG][CG]TCCT[CG]
G[CG]G[CG]GTG[CG]CAGGAGACT[CG]GG[CG]TGGGGAGGA
AGC[CG]CAGCCCAGGGCTGCT[[CG]]C[CG]CTGTTCCCCC[C
G]CCCCCTGTTGCAGGAGACAC[CG]AGGCTC[CG][CG]GAGCT
G[CG]G[[CG]]GGGGCCCA[CG]CCCAGAGAC[CG]TG[CG]AG
GAAAGCCAACCT[CG]TCCTGCCTGCCCTCCCAGC[CG][[CG]]
GGGGAGGGGGCCAC[CG]CAGTACTTCAGGACAGTTACATGGGCA
CAGCCTCCTC[CG]TCCTGG[CG]CAGGGTCAGGCTC[[CG]][C
G]GA[CG]A[CG]CCTGGAGACAGCTGCCAATGCCAATAGCTTTA
GCCCTTTATTCCCACTTAGATGATGGCCTGGCCTCTCCAGACCCC
TCTCCCCTCCCAGACATCCCCTGTGAGAAAGGAGGGAGAGGGTGT
TCCTGACCTTCAGGTGAGTTATTTATTGGCTAGGGACTGCAGTAT
TTTTTTTTTCTTTTTGGAAAGAAAATAAAATCAAGTTGGTGGTGT
CTTCAAGTCATGTTAATAGAGAAGATCTGAATTAATGCTTGATAA
GAGAAGT
SEQ ID NO: 5207
TGCCCCACCCCAACCTGAGGTCAGAGCTGAGG[CG]TAGGGGC[C
G]GGGTAGGGGCAGAGGTGC[CG]AAA[CG]CACTAGCTGGGGAC
TCC[CG]GGGGAAGGAGATAGGAGAGACCCAAGG[CG]AGCCTTA
GGACT[CG]CCCCTATGACTATGACTA[CG][CG]CTG[CG]TC[
CG]AACCACATGTCCACCC[CG]CATTTGCCTT[CG]GGGACCAG
AC[CG][CG]CTCCCTCAGATGGCTGGGGAGGGAGCAGACTC[CG
]GGTGAAGGCTGGGGGAGGTAC[CG]GGATGC[CG]AGTA[CG]C
CAGAGCAGG[CG]GGGGATGGGTTC[CG]GCTTCCTGC[CG]CCT
[CG]GCATCT[CG]CTTTGCAC[CG]GGCAAAGAAGGGGCCA[CG
]AC[CG]G[CG]AAGAG[CG][CG]TGGAGACACAGGGACC[CG]
[CG]ATCCAGGGGCAGGAACCC[CG]CCCCCTCC[CG]GAAACCT
CC[CG]GGCCCTGAGT[CG]TGCC[CG]G[CG]CTCCCCACCCCA
CCCCACCCC[CG]C[CG]CCCCTGC[CG]TCACTCA[CG]GC[CG
]CTG[CG]GGCTGGCTAG[CG]TGTG[CG]CTCTC[CG][CG]C[
CG]CAGGCACCTGCT[CG]CTGGG[CG]CTGTG[CG]CTCACTTC
TCCCT[CG][CG]CACTGCAGACTCC[CG]G[CG]CT
SEQ ID NO: 5208
[CG]GG[CG][CG]C[CG][CG]G[CG]TGGGGACTAGGCT[CG]
GG[CG][CG][CG]TCCT[CG]G[CG]G[CG]GTG[CG]CAGGAG
ACT[CG]GG[CG]TGGGGAGGAAGC[CG]CAGCCCAGGGCTGCT[
CG]C[CG]CTGTTCCCCC[CG]CCCCCTGTTGCAGGAGACAC[CG
]AGGCTC[CG][CG]GAGCTG[CG]G[CG]GGGGCCCA[CG]CCC
AGAGAC[CG]TG[CG]AGGAAAGCCAACCT[CG]TCCTGCCTGCC
CTCCCAGC[CG][CG]GGGGAGGGGGCCAC[[CG]]CAGTACTTC
AGGACAGTTACATGGGCACAGCCTCCTC[CG]TCCTGG[CG]CAG
GGTCAGGCTC[[CG]][CG]GA[[CG]]A[CG]CCTGGAGACAGC
TGCCAATGCCAATAGCTTTAGCCCTTTATTCCCACTTAGATGATG
GCCTGGCCTCTCCAGACCCCTCTCCCCTCCCAGACATCCCCTGTG
AGAAAGGAGGGAGAGGGTGTTCCTGACCTTCAGGTGAGTTATTTA
TTGGCTAGGGACTGCAGTATTTTTTTTTTCTTTTTGGAAAGAAAA
TAAAATCAAGTTGGTGGTGTCTTCAAGTCATGTTAATAGAGAAGA
TCTGAATTAATGCTTGATAAGAGAAGT[CG]CCCATTTTTCAGCA
ACTCAGCTA
SEQ ID NO: 5209
AGGGGAAAAGGTGGGTGCACAG[CG]GGTTCTCAGAGGGTGCTCC
TGAGCCCCCTGGAGCTC[CG]GGTACT[CG]GC[CG]GC[CG]G[
CG]GCCACT[CG]GCAG[CG]CTG[CG]GGCTGC[CG]GGAACTG
TTCTC[CG]CT[CG]GGGTGCTGAAAG[CG]GA[[CG]][CG]GG
AGAG[CG][[CG]]CAGAGAAGG[CG]AGGAGC[CG]GGT[CG]G
CCAGGCTCTCCTGCAGG[CG][CG]GGTCCTGCT[CG][CG]GGG
[CG]TCTCTAGAACTCACTCCTTCTT[CG]C[CG]AACTCTTTCT
[CG]CTTCCTGCCAGGTTGCCTCATGTCTCTCTCTCTCTGAGCTG
[[CG]]AACCCCCTCCTCCCAGCCCTGA[CG]TGAG[CG]CCTAC
ACCAGC[CG]C[CG]C[CG]CAGCTGC[CG]AG[CG]GGG[CG][
CG][CG]C[CG]TTCTCCCAGC[CG][CG]TGCCTGC[CG]AGAG
GC[CG][CG]GGAC[CG]GGCA[CG][CG][CG][CG][CG]TTC
CCAGGGGCCTGGGAG[CG]GCC[CG]AGGG[CG]GCCC[CG]GG[
CG]GTG[CG][CG]CTCCC[CG]AG[CG][CG]TCCT[CG]GGGC
TG[CG][CG][CG][CG][CG]G[CG]GCAAGG[CG]AGGTGC[C
G]GCAGG[CG]GTGGT[CG][CG]CTGGTGTACAA[CG]CC[CG]
CAGCAG[CG][CG]G[CG]TTCAGGTG[CG]G[CG]T[CG]G[CG
]GGCA
SEQ ID NO: 5210
GCTCCCTTCTTCCTCTCCACTTGCTCTCAATAGAGGG[CG][CG]
CTGGG[CG]AAGTAGGGG[[CG]]TGGCAGGAGCCC[CG]CTC[C
G][CG]GATCCAGGTTGAAATTCCTCCCTCCTGGAATC[CG]AC[
CG]TGAGCATTTCTGAG[CG]GTGGTGAGGAGGAGGCCACCAAGA
CACCTCCCTCC[CG]CTGTGCAAAGGAA[CG]CAGGAGCC[CG]G
[CG]CAGGTGGTGGGCTTAC[CG][CG][[CG]]CA[CG]CCTGG
CTGGAGAGGTGA[CG]C[CG]CTGTCTGCCAACCTTTCCCAGCTT
TTCCCA[CG]ATTGGAGCCAAGAGAGGTCCC[[CG]]GGGATCTC
AGTTGGGCAATTGTAAGGGAAGGAGCTCAGGACACTGACTTCAAG
ATGAAGGGGGAGGTCAG[CG]AAGACAGTTCTAGGGTGGG[CG]G
[CG]GC[CG]CTGACAGAGCAGGAGCCACAGCCAC[CG]GGGCTT
GGAGATAGGAGCAAGT[CG]CAGG[CG]GAGGGGGCTG[CG]GGC
TGGCTGCCTGGGCAGCACAGGACTTGAGGGAGCTG[CG]GGGACT
CCTGGAGTCTCATCAGGCCTTCCAGT[CG]CTGTGGGGACCC[CG
]GCTG[CG][CG][CG]GAT[CG]CCTG[CG]CCACTGTCCCCAC
TGACC
SEQ ID NO: 5211
GGGCTCCTGGGGTCC[CG]ACCCC[CG]G[CG]CACTTGC[CG]C
ACATACC[CG]CTGCCAGAGG[CG]AGGGAG[CG]AGGTGCCCTG
GCAGATT[CG]AG[CG]GCCACCCCCAGC[CG]CTGAGC[CG]AG
CTTCTGCTGC[CG]C[CG]CTGC[CG]CTG[CG]CTCCCAGC[CG
]CTGCCTG[CG]CCCTCCCTGC[CG]CCCCCT[CG]GGTG[CG]G
GCTCCAGCCCC[CG][CG]CTGGGGAGC[CG]G[CG]AG[CG]TG
AGG[CG][CG]GGCCAGGGGCTG[CG]GG[CG]CAGGACCCCAGC
CC[CG]GATCACCTGGGCT[CG]C[CG][CG]CAT[CG]TCCTTC
TCCCCAAC[[CG]]AGC[CG]GACTCCA[[CG]]GGCTCAACAGC
CCCCAGCCC[CG]GTGGTCCC[CG]CTGCCCTGCC[CG]CTGC[C
G]CCCCCACTGGG[CG]CCAGGGACAG[CG]GGGGTGC[CG]CTC
TGC[CG]GCAGGG[CG]CACAGGGGAGTGGGCACAGAGG[CG]GG
CTGGAG[[CG]]ATCTCACC[CG]TTC[CG]GGCTGGCCAGCA[C
G][CG]CCCTCCCTGGC[CG]GAG[CG]CTGCTCATCTGATGGAG
GGCAGGAAGGAGCCCTGGGCTGACTGCAGGCTCCTGGGTGACCTT
GGACAAGTTGGTTAGCCTCTCTGAGTGCCCACTTCCTCT
SEQ ID NO: 5212
G[CG]GTTT[CG]GAGGGAGCTGGGCTGTGGAGGGAGGG[CG]GC
CTCTTCACCAG[CG]T[CG]TC[CG]ACCACCC[CG][CG]CCCT
CCTGGTGGCTCTCCCA[CG]CCCCATT[CG]CACCCCAGGTCCT[
CG]GGGTGTCTCTGGTGTTGCTTCAAGTTAGGCAAGAGGAGCTGG
GGG[CG][CG]GGGAGCC[CG]G[CG]C[CG]CAG[CG][CG]TG
[CG]TGGCTGGGG[CG]CCC[CG]GGGC[CG]GCTGCACCTTCAG
T[CG]CCCCCTGGC[CG]GCCTGCCTGCCT[CG]ACCTC[CG]CA
TCCCCTTGCAC[CG]TC[CG]CT[CG][CG]TGCTG[CG]GGGCA
CT[CG]CAGCACTACCTT[CG]GTGGGGC[CG]CAGCACCCTGAG
CTCC[CG]GGACTCCCCCAC[CG]CCCC[CG]CACCCA[CG]ACC
[CG][CG]TGGTCCCAGCCCCTCCACCCTCTTCA[CG]CACCCT[
CG]CACCCTTTTGTG[CG]ACCC[CG]TGCCCTCTC[CG]GGCTG
CCAACCCCTGCCACCCAGGTGCCAGTCC[CG]GG[CG][CG]GGG
AGGAGGC[CG]GTGGGGAAGGGGG[CG]GAGGGGC[CG]GGGTGG
AGGAGGAGCTG[CG][CG]AGGGGCCTGGGGG[CG]GGGAG[CG]
AGGAGAG[CG]CT[CG]GAATGTGTGC[CG]GTCTCTAGGT
SEQ ID NO: 5213
CCCAGGTCCT[CG]GGGTGTCTCTGGTGTTGCTTCAAGTTAGGCA
AGAGGAGCTGGGGG[CG][CG]GGGAGCC[CG]G[CG]C[CG]CA
G[CG][CG]TG[CG]TGGCTGGGG[CG]CCC[CG]GGGC[CG]GC
TGCACCTTCAGT[CG]CCCCCTGGC[CG]GCCTGCCTGCCT[CG]
ACCTC[CG]CATCCCCTTGCAC[CG]TC[CG]CT[CG][CG]TGC
TG[CG]GGGCACT[[CG]]CAGCACTACCTT[CG]GTGGGGC[CG
]CAGCACCCTGAGCTCC[CG]GGACTCCCCCAC[CG]CCCC[CG]
CACCCA[CG]ACC[CG][CG]TGGTCCCAGCCCCTCCACCCTCTT
CA[CG]CACCCT[CG]CACCCTTTTGTG[CG]ACCC[CG]TGCCC
TCTC[CG]GGCTGCCAACCCCTGCCACCCAGGTGCCAGTCC[CG]
GG[CG][CG]GGGAGGAGGC[CG]GTGGGGAAGGGGG[CG]GAGG
GGC[CG]GGGTGGAGGAGGAGCTG[CG][CG]AGGGGCCTGGGGG
[CG]GGGAG[CG]AGGAGAG[CG]CT[CG]GAATGTGTGC[CG]G
TCTCTAGGTGGTGCCAAATTCTGGGGCCTAGGCATTTCCCT[CG]
CTTTATGTTTTTGGTTTTTTTTCTTCCTTCAATCTCTTTGATTAG
GC[CG]TA[CG]TGGCTGTGGCAAGGAGT
SEQ ID NO: 5214
CT[CG]GGGTGTCTCTGGTGTTGCTTCAAGTTAGGCAAGAGGAGC
TGGGGG[CG][CG]GGGAGCC[CG]G[CG]C[CG]CAG[CG][CG
]TG[CG]TGGCTGGGG[CG]CCC[CG]GGGC[CG]GCTGCACCTT
CAGT[CG]CCCCCTGGC[CG]GCCTGCCTGCCT[CG]ACCTC[CG
]CATCCCCTTGCAC[CG]TC[CG]CT[CG][CG]TGCTG[CG]GG
GCACT[CG]CAGCACTACCTT[CG]GTGGGGC[CG]CAGCACCCT
GAGCTCC[CG]GGACTCCCCCAC[CG]CCCC[CG]CACCCA[CG]
ACC[CG][CG]TGGTCCCAGCCCCTCCACCCTCTTCA[[CG]]CA
CCCT[CG]CACCCTTTTGTG[CG]ACCC[CG]TGCCCTCTC[CG]
GGCTGCCAACCCCTGCCACCCAGGTGCCAGTCC[CG]GG[CG][C
G]GGGAGGAGGC[CG]GTGGGGAAGGGGG[CG]GAGGGGC[CG]G
GGTGGAGGAGGAGCTG[CG][CG]AGGGGCCTGGGGG[CG]GGGA
G[CG]AGGAGAG[CG]CT[CG]GAATGTGTGC[CG]GTCTCTAGG
TGGTGCCAAATTCTGGGGCCTAGGCATTTCCCT[CG]CTTTATGT
TTTTGGTTTTTTTTCTTCCTTCAATCTCTTTGATTAGGC[CG]TA
[CG]TGGCTGTGGCAAGGAGTTGGGGAAA
SEQ ID NO: 5215
AATCCTACAGGTGGCTGTGACC[CG]GGCCCCTAC[CG]TTTTCC
TGGACCCTCCTTGCCTGCTCCATTTTCCAAGCCCTCCCCAATTGC
TGCAGACTGGAGTAGCC[CG]GTCTTCACTCCAATCT[CG]CTCT
CCCCTCTCCTCTCCACTTCCTCTCAAGC[CG]AGCTTGGTCATGA
GTTAG[CG]CCCATAG[CG]ATGT[CG]GAGAGTTCCTGGGGAGA
GGGGCCAGGTGATGGGAGTGTCTGTAGGGTGGGCTTGAACAGAGA
GGGTGTCTTTTCCCTTAGGGGAGGAACACCCATGCTGAAGTCCCT
T[CG]CTCTG[CG]CCTAGCTTGGCACCCCT[CG]GG[CG]TCCT
TAGCC[CG]AGGTCCCAGCCAGCCCTGGAGCT[CG]GGAGAG[CG
]G[CG]TGCACCATCCCAGTTTAAC[CG][CG]CCTGCAGTGCCC
AGC[CG]GGATGAGCACTTGGCC[CG]AGGCTGCTCACCAGG[[C
G]]CCTCCTCTCCCTTCC[CG]G[CG]CCTACTCTGGCTTGGAAG
[CG][CG]GGCCCCAC[CG]AGC[CG]CAGGGAAACCTCTGCTTC
TTTC[CG]ACTTAGAAGCCTAGACCCCATTTTCTTGGATCCTCTG
GACTCCAGCAGAGGGAAATTT[CG]CAACT
SEQ ID NO: 5216
C[CG]ACCCC[CG]G[CG]CACTTGC[CG]CACATACC[CG]CTG
CCAGAGG[CG]AGGGAG[CG]AGGTGCCCTGGCAGATT[CG]AG[
CG]GCCACCCCCAGC[CG]CTGAGC[CG]AGCTTCTGCTGC[CG]
C[CG]CTGC[CG]CTG[CG]CTCCCAGC[CG]CTGCCTG[CG]CC
CTCCCTGC[CG]CCCCCT[CG]GGTG[CG]GGCTCCAGCCCC[CG
][CG]CTGGGGAGC[CG]G[CG]AG[CG]TGAGG[CG][CG]GGC
CAGGGGCTG[CG]GG[CG]CAGGACCCCAGCCC[CG]GATCACCT
GGGCT[CG]C[CG][CG]CAT[CG]TCCTTCTCCCCAAC[CG]AG
C[CG]GACTCCA[CG]GGCTCAACAGCCCCCAGCCC[CG]GTGGT
CCC[CG]CTGCCCTGCC[CG]CTGC[CG]CCCCCACTGGG[CG]C
CAGGGACAG[CG]GGGGTGC[CG]CTCTGC[CG]GCAGGG[CG]C
ACAGGGGAGTGGGCACAGAGG[CG]GGCTGGAG[[CG]]ATCTCA
CC[CG]TTC[[CG]]GGCTGGCCAGCA[CG][CG]CCCTCCCTGG
C[CG]GAG[CG]CTGCTCATCTGATGGAGGGCAGGAAGGAGCCCT
GGGCTGACTGCAGGCTCCTGGGTGACCTTGGACAAGTTGGTTAGC
CTCTCTGAGTGCCCACTTCCTCTATTCAAATATAATA
SEQ ID NO: 5217
[CG]TCCAGAACACAGAAAATAGACTCATCTCCTAATT[CG]CCA
GGGAGCT[CG]AGGGCTG[CG]GGGC[[CG]][CG]GGGCTGCCT
CCCC[CG]CTCCTCCCCCAACC[CG]ACCCCACCCCACCCC[CG]
CCAGGGCTT[CG]G[CG]GCCTCC[CG]GAGTCACACAGCCTACC
CCCCCACCCCAACACCCCCTCCCC[CG]GCAGACAAAGGGCCTGG
GCAAATT[CG]C[CG]CC[CG]GCCTCCTAG[CG]CTC[CG]GGG
AGGC[CG]CTG[CG]CCC[CG]GAGTGGAT[CG][CG]CTGGAGG
[CG]TG[CG]C[CG]GG[CG]AGAAGC[CG][CG]GC[CG][CG]
GGAG[[CG]]CAGTATGGGGAAGAAC[CG][[CG]]AGATG[CG]
CCTGACTCACATCTGCTGCTGCTGCCTCCTTTACCAGCTGGGGTT
CCTGT[CG]AATGGGAT[CG]TTTCAGGTAAGTTCTTC[CG]TGA
CTTTCT[CG]CTTCCTTTCCAGCCATCCCAG[CG]GAGAC[CG][
CG]AA[CG]TA[CG]GGT[CG]GCAGGGCTGGTGCAAC[CG]GTG
[[CG]]TGGAGAGCAGGGCTCAGTGGGTAGCAATAAAAATGAGCT
TGAGGTGCAGGTTAAGGGGTCAAGTT[CG]GAAATGAAGCACACT
TCTGC[CG]GCCT[CG]TTTTAAC[CG]TATGTGG
SEQ ID NO: 5218
[CG]ATGGTCCCCCAAAACCTCAGCAGCTGCTTAAGCAAG[CG]G
CCAGATCCCACCCTGACCTGCTGTGGCCAGAGATCTAGAAATGGG
GC[CG]AGGAGAAGGACAGCTTCTGAGGCCTGACTTGCAAGCAGC
TTTCTT[CG]CCT[CG]TTCT[CG]GGGCTTCAGGGAGG[CG]G[
CG]ACTGGAGCCC[CG]CTGCCTACTCACAACAAAAGAAAGCAGG
GCTGG[CG]CCCCTACA[CG][CG]CTCCCATTCACTGC[CG]GC
[CG][CG]GGGAGAATCCTACCC[CG][CG]CCCT[CG]CCAGGG
TCC[CG]GGGAGCTCTGGGCTGG[[CG]]GAGG[CG]AACAAGAT
G[CG]GTTTGACAGAACCAGGC[CG]TGCTTCTGCAAACCTCAAT
GC[CG]CTC[CG]CCAGC[CG][CG]G[CG]GCCTCCCAGACTCC
ATTTGC[CG]GGGAGCTC[[CG]]AGC[CG][CG]CAAAATCTCT
GCAAGCTGTGCTCTGCAGAATGCAAAGC[CG]GGCTGGAATC[CG
]CCATTCC[CG]C[CG]CCCCAGC[CG]ACT[CG]GC[CG]A[CG
]AGCTCCCTCCTGAGCCTCC[CG]GGCCATCTGC[CG]GGA[CG]
GTTACCT[CG]TT[CG]GTGAAGC[CG]GTGCATTCACCACATCC
TCTGCTGCTCCTAGCAGGGAA
SEQ ID NO: 5219
TAGACTCATCTCCTAATT[[CG]]CCAGGGAGCT[CG]AGGGCTG
[CG]GGGC[CG][CG]GGGCTGCCTCCCC[CG]CTCCTCCCCCAA
CC[CG]ACCCCACCCCACCCC[CG]CCAGGGCTT[CG]G[CG]GC
CTCC[CG]GAGTCACACAGCCTACCCCCCCACCCCAACACCCCCT
CCCC[CG]GCAGACAAAGGGCCTGGGCAAATT[CG]C[CG]CC[C
G]GCCTCCTAG[CG]CTC[CG]GGGAGGC[CG]CTG[CG]CCC[C
G]GAGTGGAT[CG][CG]CTGGAGG[CG]TG[CG]C[CG]GG[CG
]AGAAGC[CG][CG]GC[CG][CG]GGAG[CG]CAGTATGGGGAA
GAAC[CG][CG]AGATG[CG]CCTGACTCACATCTGCTGCTGCTG
CCTCCTTTACCAGCTGGGGTTCCTGT[CG]AATGGGAT[CG]TTT
CAGGTAAGTTCTTC[CG]TGACTTTCT[CG]CTTCCTTTCCAGCC
ATCCCAG[CG]GAGAC[CG][CG]AA[CG]TA[CG]GGT[CG]GC
AGGGCTGGTGCAAC[CG]GTG[[CG]]TGGAGAGCAGGGCTCAGT
GGGTAGCAATAAAAATGAGCTTGAGGTGCAGGTTAAGGGGTCAAG
TT[CG]GAAATGAAGCACACTTCTGC[CG]GCCT[CG]TTTTAAC
[CG]TATGTGGAATAGTTTCCTTCTAATT
SEQ ID NO: 5220
GACTCATCTCCTAATT[CG]CCAGGGAGCT[CG]AGGGCTG[CG]
GGGC[CG][CG]GGGCTGCCTCCCC[CG]CTCCTCCCCCAACC[C
G]ACCCCACCCCACCCC[CG]CCAGGGCTT[CG]G[CG]GCCTCC
[CG]GAGTCACACAGCCTACCCCCCCACCCCAACACCCCCTCCCC
[CG]GCAGACAAAGGGCCTGGGCAAATT[CG]C[CG]CC[CG]GC
CTCCTAG[CG]CTC[CG]GGGAGGC[CG]CTG[CG]CCC[CG]GA
GTGGAT[CG][CG]CTGGAGG[CG]TG[CG]C[CG]GG[CG]AGA
AGC[CG][CG]GC[CG][CG]GGAG[[CG]]CAGTATGGGGAAGA
AC[CG][[CG]]AGATG[CG]CCTGACTCACATCTGCTGCTGCTG
CCTCCTTTACCAGCTGGGGTTCCTGT[CG]AATGGGAT[[CG]]T
TTCAGGTAAGTTCTTC[CG]TGACTTTCT[CG]CTTCCTTTCCAG
CCATCCCAG[CG]GAGAC[CG][CG]AA[CG]TA[CG]GGT[CG]
GCAGGGCTGGTGCAAC[CG]GTG[[CG]]TGGAGAGCAGGGCTCA
GTGGGTAGCAATAAAAATGAGCTTGAGGTGCAGGTTAAGGGGTCA
AGTT[CG]GAAATGAAGCACACTTCTGC[CG]GCCT[CG]TTTTA
AC[CG]TATGTGGAATAGTTTCCTTCTAATTAG
SEQ ID NO: 5221
CAAGGTGGGAGGCTGTG[CG]GCCCCAGG[CG][CG]G[CG][CG
]CTC[CG]C[CG]TGGG[CG]GG[CG]G[CG]ATCCCTGAGTC[C
G]GCACCTGTG[CG]C[CG]CTCCCTCTGTGCCTCC[CG]GGCAG
CCC[CG]CCTGC[CG]GCCT[CG]GAGTC[CG][CG]G[CG]C[C
G]G[[CG]]GCTAGAGGTCCAGAGG[[CG]]AACCACTTGCTGGT
GCAGAAGAGAAACCCTCAAATCCCTGGCCTTT[CG][CG]GAGA[
CG]CCTGGA[CG]GGGC[CG]TG[CG]C[CG]TGGACTGAGCAGG
[CG]TCT[CG]GGGAGCACTTCTGCAGAG[CG]AGGACTTCCATG
TGAG[CG]ATTC[CG]TTCTCCCCACCACCAATC[CG]ACCTCCC
AGC[CG]TCTC[CG]C[CG]CC[CG]AGCATCCTTGAGGTGGGA[
CG]AGCAGGGGCTTGGATCCCTGC[CG]GC[CG]TCTGGTGTGTG
AGGCTTGCA[CG]GCCCCTGGCTGCCC[CG][CG]CCT[CG]C[C
G]GAGCC[CG]AGGGGG[CG]CAGGTC[CG]GGG[CG]AGGGC[[
CG]]GC[[CG]]GGCTGTTTGATGGCTTCACTGAGAAGAGTCAAA
GTGCTGTTGGTGTTGAACTTGAT[CG][CG]GTAGC[CG]GCTT[
CG]TGCTCTTCCTGGCCAAGTGC[CG]GCCCAT[CG][CG]GTG[
CG]CAG[CG]GAGA
SEQ ID NO: 5222
GAATGTCAAGCCTCCAGGTTCACAAATGGCTGGAGGTGCT[CG]G
GGTTAGCTCC[CG]AGGG[CG]GCAAG[CG]C[CG]AC[CG]GTC
CT[[CG]]CT[CG]CTAGGCAGG[CG]CCTC[CG]GATTC[CG]T
TCATGCCTCT[CG]GAGCTACAGGGCTTGGTG[CG]GAGA[CG]C
AGGG[CG]GG[[CG]][CG]CTGGGCTCTGGGTGTC[[CG]]CAA
CCCAAAGTGCAGCTGGTGTCTTGA[CG]CTAC[[CG]]TCTATGC
ACAAGC[CG]G[CG]G[CG]G[CG]G[[CG]]GAGCTTGGAGGAG
GCAGGGAGATTCCAAATAAATCCACTAATTCTGGGCTGAG[CG]C
TC[[CG]]G[CG]TG[CG]AGCC[CG]CCTGCCTGCCAAATGTAA
CTGTGAAGTGATGTGGAAAAGTGAGC[[CG]]GGTGCCTCCCCTG
A[CG]GATCTCATTGGGCAGAG[CG]CAGAGGCAGGTCTACCACT
TGGAAATTCATGTAAGCAGTCCCCCCTCCC[CG]CCCCCTCCC[C
G]CCTTCCCCCTT[CG]CTCTCC[CG][CG]CTGGCT[CG]AT[C
G]CTCTCTTTCCCCCTACCCAAGTGGG[CG]CACAACTTTT[CG]
CTCCT[CG]CTCC[CG]AGCAAGCCC[CG][CG][CG]TCTCTGC
[CG]CCAGAGCCA[CG]AGGTGAAGATG[CG]CCCTTCC
SEQ ID NO: 5223
[CG]GCAAG[[CG]]C[CG]AC[CG]GTCCT[CG]CT[CG]CTAG
GCAGG[CG]CCTC[CG]GATTC[CG]TTCATGCCTCT[CG]GAGC
TACAGGGCTTGGTG[CG]GAGA[CG]CAGGG[CG]GG[[CG]][C
G]CTGGGCTCTGGGTGTC[[CG]]CAACCCAAAGTGCAGCTGGTG
TCTTGA[CG]CTAC[[CG]]TCTATGCACAAGC[CG]G[CG]G[C
G]G[CG]G[[CG]]GAGCTTGGAGGAGGCAGGGAGATTCCAAATA
AATCCACTAATTCTGGGCTGAG[CG]CTC[[CG]]G[CG]TG[CG
]AGCC[CG]CCTGCCTGCCAAATGTAACTGTGAAGTGATGTGGAA
AAGTGAGC[[CG]]GGTGCCTCCCCTGA[CG]GATCTCATTGGGC
AGAG[CG]CAGAGGCAGGTCTACCACTTGGAAATTCATGTAAGCA
GTCCCCCCTCCC[CG]CCCCCTCCC[CG]CCTTCCCCCTT[CG]C
TCTCC[CG][CG]CTGGCT[CG]AT[CG]CTCTCTTTCCCCCTAC
CCAAGTGGG[CG]CACAACTTTT[CG]CTCCT[CG]CTCC[CG]A
GCAAGCCC[CG][CG][CG]TCTCTGC[CG]CCAGAGCCA[CG]A
GGTGAAGATG[CG]CCCTTC[CG][CG][CG]GTG[CG][CG]CA
GCC[CG]CTGAGGAC[CG]AGCTCCTGGATAAGTGGGCTGGCAGA
TAAT
SEQ ID NO: 5224
TCTTGCACACAAAGGG[[CG]]AAGAGAGGGG[CG]TGGGGAGGC
TGGAAAGCATGGTTGCCC[CG]CCTGGCC[CG]G[CG]A[CG]CC
[CG]CTCAGCAGCCTGCTGAGGAGTGGGGA[[CG]]AAGAGCAGC
CTAAACTTAGGGCT[CG]GGATATTT[CG]ATGCCACCCAAATTG
C[CG]TCCTACCCCAA[CG]AGGCAGGGAAAGGAG[CG]GAG[CG
][CG][CG][CG][CG]AGCTGAGTGAGTGCTTA[CG]T[CG]CA
G[CG]AGATCTGTGCTGGGATAATTAGAGAGGAGTTGGGCTGAGC
[CG]AGTCCTCTTTCAGCAGCAGCAGC[CG]GAGC[CG]C[CG]C
[CG]CAGCC[CG]GTGGGGCAACCCTGACT[CG]GAC[CG]CT[C
G]GGAGAGCCCCAGGAGAGGCCAG[CG]C[CG][CG]CAGCAGC[
CG]CCC[CG]CTG[CG]CCCACCTCCC[CG]GCTGCTCC[CG]GA
GGGCTCACAAAGG[CG]GTGGC[CG]CC[CG]AGTGGCCTTCTCC
ATCCAGG[CG]TT[CG][CG]TCCTCCTCCCCACCTTCTCTCC[C
G]AAGG[CG]AAAATGGCAGGGCCAGG[CG]AGAACCTGGGACAG
[[CG]]GTGGCCCTAGCCCTG[[CG]]ATCCTCACCCCTCCTGCT
AGGAGAGGCTG[CG]GGCTGCC[CG][CG]GA[CG]ATG
SEQ ID NO: 5225
CAACCA[CG]CCCTTGAG[CG]CTG[CG]GCTT[CG]GGAAGAAA
ACAGCTGCTGCTGTCAGGCCAGGCCTGGCTC[CG]CAGCC[CG]G
AGGGCCACCAGG[CG]GCTGGCATAGGC[CG]GGGAGGGGCTGGG
AT[CG]GTGGCTG[CG]ATGCCCTGTAGAGC[CG]AGGGAAGG[C
G][CG]AGTGCA[CG]TTAGAGTGACAATATTGGC[CG]GAC[CG
]AGCCCCAAT[CG]GGGAGCTCA[CG]GCCAGCTGAATT[CG]CT
GA[CG]TGTAGGAGAGGAAAGGACCC[CG]AGAACC[CG]GAAGC
CTAGATTCCTGC[CG]GAGCTGCAAGTGCTG[[CG]]GAAATGGG
GGAAGAAGGTTTCTGGG[[CG]]CTTTAAACAAATGGCTGCCTCC
CAG[CG]CTCTGAGTTAAGGGAC[[CG]]GCTACCTAG[[CG]]T
CTAGCTGAGGAGGAAGA[CG][CG]CAGCTGGAGAACTGTTGCCT
TTGTAGTTGCTTCTCC[CG]C[CG]CATCCAGGAAAAACAGG[CG
]CTTTGGGGCTGGTTAGAACAAACAAAGCCCCAATTCC[CG]AGC
CCTGTTGAGGCT[CG]GACAGAGAGGTTTG[CG]CACAACCTG[C
G]CTTCTG[CG]CAATCAG[CG]GCTCCTGAGCC[CG]GGTCTC[
CG]GCACCACCTGGT
SEQ ID NO: 5226
TGCACACAAAGGG[[CG]]AAGAGAGGGG[CG]TGGGGAGGCTGG
AAAGCATGGTTGCCC[CG]CCTGGCC[CG]G[CG]A[CG]CC[CG
]CTCAGCAGCCTGCTGAGGAGTGGGGA[[CG]]AAGAGCAGCCTA
AACTTAGGGCT[CG]GGATATTT[CG]ATGCCACCCAAATTGC[C
G]TCCTACCCCAA[CG]AGGCAGGGAAAGGAG[CG]GAG[CG][C
G][CG][CG][CG]AGCTGAGTGAGTGCTTA[CG]T[CG]CAG[C
G]AGATCTGTGCTGGGATAATTAGAGAGGAGTTGGGCTGAGC[CG
]AGTCCTCTTTCAGCAGCAGCAGC[CG]GAGC[[CG]]C[CG]C[
CG]CAGCC[CG]GTGGGGCAACCCTGACT[CG]GAC[CG]CT[CG
]GGAGAGCCCCAGGAGAGGCCAG[CG]C[CG][CG]CAGCAGC[C
G]CCC[CG]CTG[CG]CCCACCTCCC[CG]GCTGCTCC[CG]GAG
GGCTCACAAAGG[CG]GTGGC[CG]CC[CG]AGTGGCCTTCTCCA
TCCAGG[CG]TT[CG][CG]TCCTCCTCCCCACCTTCTCTCC[CG
]AAGG[CG]AAAATGGCAGGGCCAGG[CG]AGAACCTGGGACAG[
[CG]]GTGGCCCTAGCCCTG[[CG]]ATCCTCACCCCTCCTGCTA
GGAGAGGCTG[CG]GGCTGCC[CG][CG]GA[CG]ATGTGG
SEQ ID NO: 5227
TGGGAGC[CG]C[CG]C[CG]G[CG]CCAACTTTTCCCAT[CG][
CG]GGAG[CG]AAGAGCAG[CG]GAGAGAGGGGTCCCAGAA[CG]
AAGGTGG[CG]GCA[CG]AGCTCTG[CG]CTGG[CG]GCTGTGGG
GGGC[[CG]]G[[CG]]CTCAGGACCCCAACTCCATCCAAGTTG[
[CG]]C[CG][CG]GTGGGGG[CG]GG[CG]GAGG[CG]G[CG]C
[CG]GGCAGGTGG[CG]GC[CG]CTTGCC[CG]GGCTGGGCTTGT
GC[CG]AG[CG][CG]GGTC[CG]GA[CG]GAG[CG]AGC[CG]C
[CG][CG][CG][CG]GGGGTGGAGCTGAGCTGAAGTCACTGG[C
G]GAG[CG]GG[CG][CG]GG[[CG]]CTGG[[CG]]GG[[CG]]
A[CG][CG]GGAGGGAGGGCAGG[CG]CTGC[CG]GGACTC[CG]
GG[CG]C[CG][CG]TCC[CG][CG]GC[CG]AGCTG[CG]CTG[
CG]CAC[CG]GGTCTC[CG]GG[CG]CTGGAGG[CG]C[CG]C[C
G]G[CG]ACCTCCCCT[CG]GCCT[CG]CAGG[CG]CTGC[CG]C
[CG]CCTCTCCC[CG][CG]CCCACTCTG[CG]CCCC[CG][CG]
GC[CG][CG]GGTCC[CG]G[CG]CTGAGTC[CG][CG]CACCCT
GCC[CG][CG]GGGG[CG]GT[CG]ACCC[CG]GGCTCC[CG]GA
GCAGT[CG]ATGTGT[CG]GGAGCTTCCTATTCCTGCTTTATTGT
TTCTATGAGTCTATGTTGCTG
SEQ ID NO: 5228
TCCCTG[CG]CAA[CG]CCTCCT[CG]TCCTC[CG]AGATTTTCC
AATGGTGG[CG]CAAGCTCCTCCCT[CG]CC[CG]AGCTGGG[CG
]C[CG]TCCTAGAGCCAAG[CG]CC[CG]CTAGAGC[CG]AGACT
AGGGGG[CG]GTGAGGAGTGGGACC[CG]CT[CG][CG]GCCATT
GGTTGTG[CG]GCCAGGAGGGG[CG]GAGGGA[CG]A[CG][CG]
[CG]GGCTGTG[CG]GGGC[CG]GCAGCC[CG]GATCCACTCC[C
G]CTG[CG]CCTGCTGTC[CG]C[CG]TG[CG]CCCAGACTG[CG
][CG]C[CG][CG]C[CG]CTG[CG]CCCAACATTCC[CG]AGGA
[CG]GCTT[CG][CG]GG[CG][[CG]]TAT[CG]TCCAGAC[CG
]GAGCAC[CG]CCCCAC[CG]CTAG[CG]CAGGAGACCTGC[CG]
GGGAAGT[CG][[CG]]TGTCCTGACCTGCAGGTCAGTGAGGGAA
ACTTGCAGGGTCC[CG]AGCTCTGGGGGTGAACTTGCCCTA[CG]
[CG]GAGAG[CG]CCCCACTCCTTGCCTGGGG[CG]GCCT[CG]G
GTCCTGGAGG[[CG]]AGC[CG]CCTCCCAGACTCTCCCC[CG]A
GATCTGGGCTCC[CG]GAGATGTGGCTCCTTTCTGAGAAACACCC
CCAAATCTATCCCTTTTGAGGTGTCCTTACTGTCC[CG]AGAAGC
AAT[CG]CAG
SEQ ID NO: 5229
AGC[CG]C[CG]C[CG]G[CG]CCAACTTTTCCCAT[CG][CG]G
GAG[CG]AAGAGCAG[CG]GAGAGAGGGGTCCCAGAA[CG]AAGG
TGG[CG]GCA[CG]AGCTCTG[CG]CTGG[CG]GCTGTGGGGGGC
[[CG]]G[CG]CTCAGGACCCCAACTCCATCCAAGTTG[CG]C[C
G][CG]GTGGGGG[CG]GG[CG]GAGG[CG]G[CG]C[CG]GGCA
GGTGG[CG]GC[CG]CTTGCC[CG]GGCTGGGCTTGTGC[CG]AG
[CG][CG]GGTC[CG]GA[CG]GAG[CG]AGC[CG]C[CG]C[CG
][CG][CG]GGGGTGGAGCTGAGCTGAAGTCACTGG[CG]GAG[C
G]GG[CG][CG]GG[[CG]]CTGG[[CG]]GG[[CG]]A[[CG]]
[CG]GGAGGGAGGGCAGG[CG]CTGC[CG]GGACTC[CG]GG[CG
]C[CG][CG]TCC[CG][CG]GC[CG]AGCTG[CG]CTG[CG]CA
C[CG]GGTCTC[CG]GG[CG]CTGGAGG[CG][CG]C[CG]G[CG
]ACCTCCCCT[CG]GCCT[CG]CAGG[CG]CTGC[CG]C[CG]CC
TCTCCC[CG]C[CG]CCCACTCTG[CG]CCCC[CG][CG]GC[CG
][CG]GGTCC[CG]G[CG]CTGAGTC[CG][CG]CACCCTGCC[C
G][CG]GGGG[CG]GT[CG]ACCC[CG]GGCTCC[CG]GAGCAGT
[CG]ATGTGT[CG]GGAGCTTCCTATTCCTGCTTTATTGTTTCTA
TGAGTCTATGTTGCTGTTTC
SEQ ID NO: 5230
[CG]C[CG][CG]G[CG]CCAACTTTTCCCAT[CG][CG]GGAG[
CG]AAGAGCAG[CG]GAGAGAGGGGTCCCAGAA[CG]AAGGTGG[
CG]GCA[CG]AGCTCTG[CG]CTGG[CG]GCTGTGGGGGGC[[CG
]]G[[CG]]CTCAGGACCCCAACTCCATCCAAGTTG[[CG]]C[C
G][CG]GTGGGGG[CG]GG[CG]GAGG[CG]G[CG]C[CG]GGCA
GGTGG[CG]GC[CG]CTTGCC[CG]GGCTGGGCTTGTGC[CG]AG
[CG][CG]GGTC[CG]GA[CG]GAG[CG]AGC[CG]C[CG]C[CG
][CG][CG]GGGGTGGAGCTGAGCTGAAGTCACTGG[CG]GAG[C
G]GG[CG][CG]GG[[CG]]CTGG[CG]GG[CG]A[[CG]][CG]
GGAGGGAGGGCAGG[CG]CTGC[CG]GGACTC[CG]GG[CG]C[C
G][CG]TCC[CG][CG]GC[CG]AGCTG[CG]CTG[CG]CAC[CG
]GGTCTC[CG]GG[CG]CTGGAGG[CG]C[CG]C[CG]G[CG]AC
CTCCCCT[CG]GCCT[CG]CAGG[CG]CTGC[CG]C[CG]CCTCT
CCC[CG]C[CG]CCCACTCTG[CG]CCCC[CG][CG]GC[CG][C
G]GGTCC[CG]G[CG]CTGAGTC[CG][CG]CACCCTGCC[CG][
CG]GGGG[CG]GT[CG]ACCC[CG]GGCTCC[CG]GAGCAGT[CG
]ATGTGT[CG]GGAGCTTCCTATTCCTGCTTTATTGTTTCTATGA
GTCTATGTTGCTGTTTCCTT
SEQ ID NO: 5231
CAG[CG][CG]CAGCTAAAGGGTTTGGAGGTGAAAAGGAGGGCTG
GGTGGGGAGGAGAGAAAGTGAGAAGGGAGAGAAAAG[CG]G[CG]
GCTGAGCAAAG[CG]GGAGC[CG]G[CG][CG]GG[CG]GTGGGG
CCTGACC[CG]GA[CG]C[CG][CG]CACC[CG]GCTC[CG]CCC
[CG][CG]CCCAG[CG]CCC[CG]CTCACTCCCTGGC[CG][CG]
CCTGCCTT[CG][CG]CCCCTCTGTTTTCTCTCACTTACCAGCAG
CTCTGCTGACAAG[CG]AGAAAACTGACAAGTGAGAAAAGT[[CG
]]TCTGTCAG[CG]CC[CG]GGCTGGTGTCTGGGCTGTCAC[CG]
[CG]GG[CG][CG]GAGGGAGGGG[CG][CG][CG]GGCAC[CG]
C[CG]TCTGC[CG]GGCACA[CG]CTGGGCTC[CG]ACCC[CG]G
C[CG]GGCACACTCACCCC[CG]CAGGGCAGC[CG][CG]GCCAC
CAC[CG]ACCTG[CG]GAGCTG[CG]TGGGCTTC[CG]GAACCCT
CACCTCAACAAGAGCT[CG]GCTC[CG][CG][CG]CAAGTAGGA
GGGAAGGGAAATCCTTATCCTCC[CG]GGGCCAGGGCA[CG]CAG
AAGC[CG][CG]AGGCTT[CG][CG]GAGGTTGGGCCTGGAGGTC
CCCCACAGCCTCACTCT[CG]CACC[CG]CCACAGAAC[CG]GAT
CCAT
SEQ ID NO: 5232
CCAGGAGACCAGGGTAC[CG]GGT[CG][CG]TGTC[CG]C[CG]
C[CG]C[CG]CC[CG]CTGGAATCCAGGGCTGCA[CG][CG]TGA
C[CG][CG]G[CG]G[CG]G[CG]G[CG]CTGG[CG]AGGGGAAG
GGGGAGGGGG[CG]CTCCCT[CG][CG]CACCAGATTATTTTTGG
CTC[CG]CAGC[[CG]]GGGCTGCT[CG]CTGCTTGT[CG][[CG
]][CG]CTCACACACACACAGACACACA[CG]CACACACACACAT
GCACACATTTTCT[CG][CG]CTCTCTC[CG]GCTCTCCTTTGTT
TATTTTCTAATCTATATTTTTACTGGAAGATTTCCTCTTTATTCT
CTCC[CG]CCCTCCTACAAG[CG]CTCTTGCTGGC[CG]TCTGGG
TGCACACAC[CG]CTCCCT[CG]ATCACCCCAGCCCCCTTCCTGG
TCTCC[CG]AG[CG][CG]GGGTTTGAAGGTCACCTCCTTTCCAG
TCCC[CG]TG[CG]AGC[CG][CG]CTGC[CG]C[CG]CCTCCTC
CAGCCAGAGT[CG]GTGGGACTGGCTG[CG]CTGCCCTGAAGTGG
TTCTCCAAGCAG[[CG]][[CG]]GAGGGTGG[[CG]]GA[CG]G
[CG]GA[CG]GAGCCCAGGGGC[CG][CG]T[CG]GGTGGGGAAA
CC[CG]AACT[CG][CG]GAGGGGAATCCCTCCCCCTC[CG]CCC
CAGCCCCC
SEQ ID NO: 5233
CCAAACAAAGCCACAGAAG[CG]ATCAG[CG][CG]CAGCTAAAG
GGTTTGGAGGTGAAAAGGAGGGCTGGGTGGGGAGGAGAGAAAGTG
AGAAGGGAGAGAAAAG[CG]G[CG]GCTGAGCAAAG[CG]GGAGC
[CG]G[CG][CG]GG[CG]GTGGGGCCTGACC[CG]GA[CG]C[C
G][CG]CACC[CG]GCTC[CG]CCC[CG][[CG]]CCCAG[CG]C
CC[CG]CTCACTCCCTGGC[CG][CG]CCTGCCTT[CG][CG]CC
CCTCTGTTTTCTCTCACTTACCAGCAGCTCTGCTGACAAG[CG]A
GAAAACTGACAAGTGAGAAAAGT[[CG]]TCTGTCAG[CG]CC[[
CG]]GGCTGGTGTCTGGGCTGTCAC[[CG]][CG]GG[CG][CG]
GAGGGAGGGG[CG][CG][CG]GGCAC[CG]C[CG]TCTGC[CG]
GGCACA[CG]CTGGGCTC[CG]ACCC[CG]GC[CG]GGCACACTC
ACCCC[CG]CAGGGCAGC[CG][CG]GCCACCAC[CG]ACCTG[C
G]GAGCTG[CG]TGGGCTTC[CG]GAACCCTCACCTCAACAAGAG
CT[CG]GCTC[CG][CG][CG]CAAGTAGGAGGGAAGGGAAATCC
TTATCCTCC[CG]GGGCCAGGGCA[CG]CAGAAGC[CG][CG]AG
GCTT[CG][CG]GAGGTTGGGCCTGGAGGTCCCCCACAGCCTCAC
TCT[CG]C
SEQ ID NO: 5234
C[CG]GGTGCAAG[CG]ACCCCCTGCCCCTTG[CG]CCCC[CG]C
[CG]TGTGGTCCTGGGAGTG[CG]ATG[CG]TCTTAGG[CG][CG
][CG][CG]ATTCCT[CG]AAACC[CG]G[CG]CAGT[CG][CG]
ACCTCTC[CG]GCTGACTGGTC[CG]GGAGTCC[CG]GGAGCTCC
AGCAGGGCCAG[CG]TTCCCCAA[CG]GGCTGAGC[CG]CT[CG]
TCCC[CG][CG]ACCTGCATGTCCCACT[CG]GGCCC[CG][CG]
CCCCCACTTAGC[CG]CTGAAGG[CG]TCT[CG][CG]GTGACTT
TACCTTGCCTGGGGTGGAAGTCAACTTCCCAAGACCTGGTGATGA
AGAG[[CG]]AGAGATGGTAGAGCAGCCCAGTCAGCCACTTGGCC
ACCCACATGGTTCCACCCTGGGGACCC[CG]AT[CG]GGGAGGCC
CACCAGAGC[CG]C[CG]GCAGCCAAGC[CG]GCTTCCCT[CG][
CG]CTC[CG]ATCCCTCTCCTCCCTCT[CG]CC[CG]C[CG]CTC
TC[CG][CG]CCTCAGCAGCCTAGGGAACAGCAG[CG][CG]GGC
CCTGGGC[[CG]]GCTGGTCCAGCCACAC[CG]CCTGTCTC[CG]
CT[CG]CTGAGC[CG]CTGCTTCAT[CG]CACTGCAGCCTCACCC
CCCTTCCTG[CG]CC[CG][CG]CTGCCCTCAGAAACTCTCTG
SEQ ID NO: 5235
[CG][CG]C[CG]A[CG]CATG[CG]AACTTCC[CG]GTTC[CG]
GGGGAGCT[CG]GGGTGAGGGCAGC[CG]AAG[CG]AGGGGTG[C
G]G[CG]GGGGCCT[CG]G[CG]GGGGCT[CG]GATCCCATCAAC
AG[CG]CCTCTG[CG]GGTGC[CG]AGC[CG]CCCCTAATCCAGA
AGGG[CG]CTCCCAGCTGGG[CG]C[CG]GGATC[CG]GGCAAGT
TAGGCTGGTCC[CG]AGTCC[CG]AATCTCCCACCC[CG]GACCC
CCCAGACCCAGAGCTGCCC[CG][CG]GATGAGGAGGGGG[CG]T
GGCTGAGGGACA[CG]GTGTTTGTTCCCCATCCCTGAGTTC[CG]
TG[[CG]]GACCTGCCCCAGCCCCAGCACAGTCACCCTGTGGAAC
TGGCATCCAGGAGAGGTGCTC[CG]CTCTGTG[CG]CTCCTTCCT
CC[CG]GCTGTGTGCTCCCTGTCCACAGCTCCCCATCCACAGCTT
[CG]GCACTGTGGGGCTGGTGCTCCAGGAGGGAAGGCC[CG]GTT
CTTTGCATCCCCTTTC[CG]CAAC[CG]CTAGGGTCTCTGTG[CG
]GGCCCTAGAGGAA[CG]CTCACTCAT[CG][CG]CAGCTCACCA
GGGACCCCAGCTTGGCAGAAACCAGGGGCTCTCTG[CG]GGTTCT
CTCAAATGAGTCCTT
SEQ ID NO: 5236
TTCTCCCCCTACCCCCAAACAAAGCCACAGAAG[CG]ATCAG[CG
][CG]CAGCTAAAGGGTTTGGAGGTGAAAAGGAGGGCTGGGTGGG
GAGGAGAGAAAGTGAGAAGGGAGAGAAAAG[CG]G[CG]GCTGAG
CAAAG[CG]GGAGC[CG]G[CG][CG]GG[CG]GTGGGGCCTGAC
C[CG]GA[CG]C[CG][CG]CACC[CG]GCTC[CG]CCC[CG][C
G]CCCAG[CG]CCC[CG]CTCACTCCCTGGC[CG][CG]CCTGCC
TT[CG][CG]CCCCTCTGTTTTCTCTCACTTACCAGCAGCTCTGC
TGACAAG[CG]AGAAAACTGACAAGTGAGAAAAGT[[CG]]TCTG
TCAG[CG]CC[[CG]]GGCTGGTGTCTGGGCTGTCAC[[CG]][C
G]GG[CG][CG]GAGGGAGGGG[CG][CG][CG]GGCAC[CG]C[
CG]TCTGC[CG]GGCACA[CG]CTGGGCTC[CG]ACCC[CG]GC[
CG]GGCACACTCACCCC[CG]CAGGGCAGC[CG][CG]GCCACCA
C[CG]ACCTG[CG]GAGCTG[CG]TGGGCTTC[CG]GAACCCTCA
CCTCAACAAGAGCT[CG]GCTC[CG][CG][CG]CAAGTAGGAGG
GAAGGGAAATCCTTATCCTCC[CG]GGGCCAGGGCA[CG]CAGAA
GC[CG][CG]AGGCTT[CG][CG]GAGGTTGGGCCTGGAGGTCCC
CCAC
SEQ ID NO: 5237
[CG]GGCTTGGGAGC[CG]C[CG]C[CG]G[CG]CCAACTTTTCC
CAT[CG][CG]GGAG[CG]AAGAGCAG[CG]GAGAGAGGGGTCCC
AGAA[CG]AAGGTGG[CG]GCA[CG]AGCTCTG[CG]CTGG[CG]
GCTGTGGGGGGC[[CG]]G[CG]CTCAGGACCCCAACTCCATCCA
AGTTG[CG]C[CG][CG]GTGGGGG[CG]GG[CG]GAGG[CG]G[
CG]C[CG]GGCAGGTGG[CG]GC[CG]CTTGCC[CG]GGCTGGGC
TTGTGC[CG]AG[CG][CG]GGTC[CG]GA[CG]GAG[CG]AGC[
CG]C[CG]C[CG][CG][CG]GGGGTGGAGCTGAGCTGAAGTCAC
TGG[CG]GAG[CG]GG[CG][CG]GG[CG]CTGG[[CG]]GG[CG
]A[CG][CG]GGAGGGAGGGCAGG[CG]CTGC[CG]GGACTC[CG
]GG[CG]C[CG][CG]TCC[CG][CG]GC[CG]AGCTG[CG]CTG
[CG]CAC[CG]GGTCTC[CG]GG[CG]CTGGAGG[CG]C[CG]C[
CG]G[CG]ACCTCCCCT[CG]GCCT[CG]CAGG[CG]CTGC[CG]
C[CG]CCTCTCCC[CG]C[CG]CCCACTCTG[CG]CCCC[CG][C
G]GC[CG][CG]GGTCC[CG]G[CG]CTGAGTC[CG][CG]CACC
CTGCC[CG][CG]GGGG[CG]GT[CG]ACCC[CG]GGCTCC[CG]
GAGCAGT[CG]ATGTGT[CG]GGAGCTTCCTATTCCTGCTTTATT
GTTTCTATGAGTCTATG
SEQ ID NO: 5238
T[CG]GGAT[CG]G[CG]CCTCCTAGGTG[CG]GGCTGTG[CG]G
GCAGTCCTGCTCC[CG]CCCC[CG]GGGCTGAGCTGG[CG]G[CG
]T[CG]GCTTCAGAGCCT[CG]GAGC[CG]AGC[CG]AGTC[CG]
GGGAAG[CG]CTCCTTTCTCAGCTCCAGC[CG]AAGAGC[CG]CC
[CG]GCAGGGAGAGG[CG]GAGTCACTGG[CG]CC[CG]GGG[CG
]GGGTGTTGGGGAGGGGTGAGTAGGGACAGGAGGGAGGGGGTGGA
GAGTTGGGCTGCTG[CG]A[CG]CCTCC[CG]GTGGCTCTGG[CG
]GCACCAATG[CG]GGGCAC[CG]GAGCAGGTGTC[CG]ACTG[[
CG]]GTG[CG]GGCTACCACTC[CG]C[CG]GGGAACCT[CG]TC
TTGCTCCTCCCTG[CG]CTT[CG]CCTCTGGAATCTCTCCCTTAT
CTCATGC[CG][CG]GTGAAACTTTGCTCAAGTTGGTGGTGCTCT
GAGGTCTCCAGGGCTGGAGGC[CG][[CG]][CG]A[CG][CG]A
AT[CG]C[CG]ACACTGGATTCT[CG]GG[CG]TTAGTC[CG]AA
GTTTAAGTTCCCTT[CG]TTCACTCCTTTGCCCTGGAT[CG]AAC
[CG]CAGCCCCAGGA[CG]CAG[CG]GGTTCCACTGGAAA[CG]G
GGAAT[CG]GGGAGAGG[CG]GT[CG]CTGGGCTC[CG]GG[CG]
AC
SEQ ID NO: 5239
[CG]A[CG]CCTCAGCTCATTTGTTAATTAGCAGTAATTGCC[CG
]GGTCAGTGACACCTCCTACTTGAGCCTCTTTCCCTGTCCATAAA
ATGCACTAATACTGGGGTGGGTGGGATTGGGAGGTGGGTCCTGGA
GCTCCATCCAATCAGTGGCACTGGTGTAGGAAC[CG]CC[CG]AT
CAGG[CG][CG]CAGTTGGAGAGGAGGG[CG]TGGCTTC[CG]GC
ATTTGG[CG]GGGTCTTTGTCTCT[CG]CTCC[CG]CCAGAGCTT
GGGAT[CG]GTCTTCACTGTTC[CG][CG]TCCTCTACCTGGGAA
CCAC[CG]ACCCC[CG]CC[CG]G[CG]ACTTTGTCACAGACTCT
GTTGCCCTGTGATCTGCAGGTCCTGGGAGA[CG]CACAGCCAAGA
TGCCAGGGCACCCTGGAAGCTGTGAAGTGGTG[CG]TATG[CG]G
GGTTGGGCATCCCTTGAGG[CG]AGAG[CG]GGCTGTGAAACCTG
CAGGAC[CG]GCCTCCCCACAGTC[CG]CTCC[CG]GGTC[CG]C
[CG]ACCTCAGTCCTCTCTGG[CG]CAGCT[CG]GCCCCAGG[CG
]TCTCTGGC[CG]CAAGATGG[CG]GCTGGGCC[CG][CG]GC[C
G]GGACCCCCAG[CG]TCT[CG]C[CG][CG]TCCCTGCTGGG[C
G]CCCTGTC
SEQ ID NO: 5240
CCTTTGGGAAACCTTAAAAGCCTGGACTTGGAT[CG]CATCCCTC
CTTTCTT[CG]AAACTCTCCATGGCTTCTAG[CG]TCTTCACAG[
CG]AATACACAGCCCCCAGCCTGCTCTTCCAGGAGAAGCTT[CG]
CCTCACA[CG]TTGTTCCCTACAGG[CG]CCTGTGGACCCCAGGT
TCCATCCTCC[[CG]]GGTCTGTCCTCCAGGCCATAGCC[CG][C
G]C[CG]GTCCCTGTGCCTGT[CG]CTCTCCCCAC[CG]CATCCC
ACAGGTGTCAGAAACAAGGACCTCTCC[CG][CG]AA[CG]CCTC
AGTGTCC[CG]A[CG]C[CG]GGTC[CG]GGCTGCAGACCTGTGA
ACAGT[CG]C[CG]CTCTCT[CG]CTGCTTCAGGATT[CG]GGTT
AGGATGAGGTGACCTGAGGGCAGGGAAGGCACAATTACCTGAGC[
CG]GGAGCCTCAG[CG][CG]GC[CG]C[CG]CCATCC[CG]AGT
A[CG][CG]GGGAAAGCACTGC[CG]GGAG[CG]G[CG]GG[[CG
]][CG]TTA[CG]GGGCCTAAGATCTGCCTCTGTGCAGCAAGAAG
GACTCTTCACCTTCTGGGTTCAGACAC[[CG]][CG]GTT[CG]G
ACCCATAGCTCCAG[CG]CCTCTCACCTCACAAAC[CG]CAGAAA
CACACCCAACATTAAC
SEQ ID NO: 5241
G[[CG]]CCCTGTTTCACCAGGCCCAGTCACTCCAGCTCCAGCAG
CACTGAGAGCTCA[CG]AA[CG]CCAAGGG[CG]AGAG[CG]AGG
TGGGGGAGGG[CG]GC[CG]GAAATG[CG][CG][CG][CG]GCT
CTCTGGGAAATGGAGTTAGGCT[CG][CG]C[CG]G[CG]CAGTG
T[CG]GCTGC[CG]GTGC[[CG]][CG]GCCTTTGTCT[CG]CAG
TCAGGAGGGTGAGCTAGGC[CG]G[CG]AGGAGGGGGAGGGGAGG
CCAGGC[CG]GGC[CG]GGC[CG]GGC[CG]GGTG[CG]GGGGGT
C[CG]GGGATCTTCCTGAGGCCCTGG[CG]GGG[CG]AGTTTCCA
GCAG[[CG]][CG][CG]TCTGTGTGGAGTC[CG]TTTTGCTGCC
[CG]GGGCCTGGGGAAGGC[CG]TTT[CG]GGGCTGG[CG]GGGG
CAGGCTTTG[CG]GGGCATCCCTAGTCTGAGAGGAGGG[CG]GGG
CCCAA[CG]CCCAGCTGGACAGGAGCTGCTC[CG]A[CG]GCCCC
TGAGGGAA[CG][CG][CG]CCC[CG]CCCCTGGCCCCACCTCTG
CCCCACAC[CG]GGCACTGGGC[CG]CCACCTTTGTTGATGGAAG
AGAGAAACTGAGGCATAGTGCCCAGGGCCAGGGGAC[CG]CAACC
ACC[CG]GCCCTTGTCACTGTAG[CG]TTAAGGCT
SEQ ID NO: 5242
CTGGGGAGCCAC[CG]C[CG]G[CG]CCAACTTTTGCCATCC[CG
]GGAGCCATAG[CG]GCAGAGAGGGGGTCC[CG]GACC[CG]AGG
TGGCAG[[CG]]AGGGGCTCTGCTTTGGCAGCTGT[CG]GGGCTG
GAGCTCAGGACCCCAACTCCAGCCAAGT[CG]GGC[CG][CG]G[
CG]GGGCAGG[CG]TAGGCTG[CG]CAGGCAGGGGTGGC[CG][C
G]GGTGCTGAGCCAGT[CG]C[CG]C[CG][CG]CAGG[CG]GG[
CG]CTGGAGTCTCAGCTACCTGG[CG][CG]AC[CG]CCAGCAGC
ACCCAG[CG][CG]GGGC[CG]GGAGCTGCTGGGGGCCCAGGCTC
[[CG]]CTCTCCCCAC[CG]CTCTGCAC[CG]CTGC[CG]GCTG[
CG]GACAGACC[CG]ATG[CG]CCACCACCAC[CG]CAGTTGCCA
[CG]ACCCCCATGGAAT[CG]CTGG[CG]CCTC[CG]CTC[CG]G
TTCTCTAACC[CG]G[CG]CACTCTGGGATCCCAAAGCTTGC[CG
]TGGGTTGGAGGCTTCCTACTCTTTGTG[CG]TTGAGGCTTTGAC
CTGACCCAGAG[CG]CAGCTTATGCCCTGGCTTGGGCAGAAAATA
ATATTAATTACTGATCCTTTGTGCTCC[CG]GTCCTGGCTTCCTA
AAGCACTCCTCTCTCATTCAGCACC
SEQ ID NO: 5243
TCTGTCTCTACTGAGAA[CG]CAG[[CG]][[CG]]TCAGGGC[C
G]AGCTCTTCACTGGCCTGCTC[CG][CG]CTCTTCAATGCCAG[
CG]CCAGG[CG]CTCACCCTGCAGAG[CG]TCC[CG]CCTCTCAA
AGAGGGGTGTGACC[CG][CG]AGTTTAGATAGGAGGTTCCTGC[
CG]TGGGGAACACCC[CG]C[CG]CCCT[CG]GAGCTTTTTCTGT
GG[CG]CAGCTTCTC[CG]CC[CG]AGC[CG][CG][CG][CG]G
AGCTGC[CG]GGGGCTCCTTAGCACC[CG]GG[CG]C[CG]GGGC
CCT[CG]CCCTTC[CG]CAGCCTTCACTCCAGCCCTCTGCTCC[C
G]CA[CG]CCATGAAGT[CG]C[CG]TTCTAC[CG]CTGCCAGAA
CACCACCTCTGTGGAAAAAGGCAACT[CG]G[CG]GTGATGGG[C
G]GGGTGCTCTTCAGCAC[CG]GCCTCCTGGGCAACCTGCTGGCC
CTGGGGCTGCTGG[CG][CG]CT[CG]GGGCTGGGGTGGTGCT[C
G][CG]G[CG]TCCACTG[CG]CC[CG]CTGCCCT[CG]GTCTTC
TACATGCTGGTGTGTGGCCTGA[CG]GTCAC[CG]ACTTGCTGGG
CAAGTGCCTCCTAAGCC[CG]GTGGTGCTGGCTGCCTA[CG]CTC
AGAAC[CG]GAGTCTG[CG]GGTGCTTG[CG]CC[CG]C
SEQ ID NO: 5244
GTGGGGATTTTGTCTGCTTGTTCACTGCTGTGTGCCCAGGGCCCA
[CG]ACAGTGCC[CG]ATACAGCAGG[CG]CTCAATAAGTGG[[C
G]]AATGAAAGACAGGGCAAGG[CG]GAGAGAGAAGAAACAGAAA
GGGAAAGAGAAAGCC[CG]GGC[CG]CCAGAG[CG]CTTAATCAC
A[[CG]]TACATCCTAC[CG]GGCCCCTCTCCAGCTGGCTCAGGC
TCCACACTGGTCCTCCAGCTCCCTTTTCTCAG[CG]ATGGACTCA
CAGCCCCACC[CG]GAG[CG]CTGGAG[CG][CG]GA[CG][CG]
GTCACTG[CG][CG]TG[CG]CCTCAC[CG][[CG]]CTGGCACC
C[CG]GCCTGGCAGCCTTTGGGGACCTGAACCAGCTG[CG]CCTG
[CG]CAGGTGGAA[CG]GGTGGAA[CG]GGTGGGGGAG[CG]GAC
AGT[CG]AA[CG]GCCTGAGAGGGCTCAGCTGGTC[CG]GGTGGG
ATTCTGAAGGCCAG[CG]GAGGTGGGCTTACTAGGGAAAGGGG[C
G]CAGCAGTAACTACC[CG]CAACTGAG[CG]G[CG]GGATCTG[
CG]GC[CG]CC[CG]GGGCCCT[CG]GCCAACATCCCAGCATCCT
CTG[[CG]]C[CG][CG][[CG]]GCCC[CG]GACTCCATTTCCC
AG[CG]GCCCCTG[CG]GCCTGCCCTAG
SEQ ID NO: 5245
[CG][CG]CCTC[CG]GCAGTCTCAGATCC[CG]CTCCCAGGTCC
CTC[CG]CCTGGCATCCTCTGCC[CG]CC[CG]GTCACTG[CG]C
TGAGCAGGCAGG[CG]GG[CG]GGC[CG]G[CG]CCTGTCACC[C
G]CAGCTGGAG[CG]CC[CG]TTCCCTGCC[CG]CCCCATCTGCA
GGG[CG]CCT[CG]GGCACTG[CG]TC[CG]GCCC[CG][CG]AG
G[CG]GC[CG]GGATGAGAAGCAGC[CG]GAGTTCCT[CG]GGGG
CAGCAGTGACAGAGGAG[CG]A[CG][CG]CTCT[CG]CCTGGAT
TCTTTTCCTTTTCTGCCCAGG[CG]AGACCCACTTATTTCCTGGG
GT[[CG]][CG]TTGGAGAGGGCTTTCA[CG]CC[CG]GGGTG[C
G]GAAAAGAAGCCAAATCCACCTTCCTCCACTGTTGGCCACCCTC
CTGCAGGCTCCCCCAG[CG]C[CG]ACTTCATTTTCTCTCTTCTC
T[CG]CTACTTCCTCTCTGCCTTTCTTT[CG]TTATTTTTCCAC[
CG]AGT[CG]CCAC[CG]C[CG]CTTGCTTCCT[CG]GAGGAGCA
A[CG][CG]ACTGGCCAGCAAAGGGTGGCTCTGCTC[[CG]]AG[
CG]CCAAAGTTCTCCC[CG]CCTCAGC[CG]G[CG]CCTCCCTGT
TGCTGAGCCCTGG[CG]CCTGCCTGT[CG]CTTGGCT
SEQ ID NO: 5246
ACTCATAAAAGATGTAAGAGGAGGAGAAAGTTGGCAAGAAGTGGA
TGCTTTCATCTGCAGAGAAAGGCCCCTGAGCCCTAAAAAAACTGC
CCTCCTCCCCACTCCCCCCTCCTCCCCC[CG]ACACACAAAAAAG
TTCACCCACTACACCTGCCTTCCCCAGATGGGCTCCCTG[CG]CA
A[CG]CCTCCT[CG]TCCTC[CG]AGATTTTCCAATGGTGG[CG]
CAAGCTCCTCCCT[CG]CC[CG]AGCTGGG[CG]C[CG]TCCTAG
AGCCAAG[CG]CC[CG]CTAGAGC[CG]AGACTAGGGGG[CG]GT
GAGGAGTGGGACC[CG]CT[CG][CG]GCCATTGGTTGTG[CG]G
CCAGGAGGGG[CG]GAGGGA[CG]A[CG][CG][CG]GGCTGTG[
CG]GGGC[CG]GCAGCC[CG]GATCCACTCC[CG]CTG[CG]CCT
GCTGTC[CG]C[CG]TG[CG]CCCAGACTG[CG][CG]C[CG][C
G]C[CG]CTG[CG]CCCAACATTCC[CG]AGGA[CG]GCTT[CG]
[CG]GG[CG][CG]TAT[CG]TCCAGAC[CG]GAGCAC[CG]CCC
CAC[CG]CTAG[CG]CAGGAGACCTGC[CG]GGGAAGT[CG][[C
G]]TGTCCTGACCTGCAGGTCAGTGAGGGAAACTTGCAGGGTCC[
CG]AGCTCTGGGGGTGAACTTGCCCTA[CG]
SEQ ID NO: 5247
AGAGTCTGTCTCTACTGAGAA[CG]CAG[[CG]][[CG]]TCAGG
GC[CG]AGCTCTTCACTGGCCTGCTC[CG][CG]CTCTTCAATGC
CAG[CG]CCAGG[CG]CTCACCCTGCAGAG[[CG]]TCC[CG]CC
TCTCAAAGAGGGGTGTGACC[CG][CG]AGTTTAGATAGGAGGTT
CCTGC[CG]TGGGGAACACCC[CG]C[CG]CCCT[CG]GAGCTTT
TTCTGTGG[CG]CAGCTTCTC[CG]CC[CG]AGC[CG][CG][CG
][CG]GAGCTGC[CG]GGGGCTCCTTAGCACC[CG]GG[CG]C[C
G]GGGCCCT[CG]CCCTTC[CG]CAGCCTTCACTCCAGCCCTCTG
CTCC[CG]CA[CG]CCATGAAGT[CG]C[CG]TTCTAC[CG]CTG
CCAGAACACCACCTCTGTGGAAAAAGGCAACT[CG]G[CG]GTGA
TGGG[CG]GGGTGCTCTTCAGCAC[CG]GCCTCCTGGGCAACCTG
CTGGCCCTGGGGCTGCTGG[CG][CG]CT[CG]GGGCTGGGGTGG
TGCT[CG][CG]G[CG]TCCACTG[CG]CC[CG]CTGCCCT[CG]
GTCTTCTACATGCTGGTGTGTGGCCTGA[CG]GTCAC[CG]ACTT
GCTGGGCAAGTGCCTCCTAAGCC[CG]GTGGTGCTGGCTGCCTA[
CG]CTCAGAAC[CG]GAGTCTG[CG]GGTGCTTG[CG]C
SEQ ID NO: 5248
TC[[CG]]GCACAGCAATTAACATTGGTCCAAA[[CG]]GTGTCA
GTGAACTTTAGCTGCAAAGAAACTACCTCATTTAAAATTTAAGCA
GACCACAGGGAGTAGCATTGGAGGAAAGAGAGGAGATTTTCTCCT
TCAACATTTTGAGCATACTGGAATGCCAACCCCCTAGGTAGGTGC
CAGGGCCACCACAGGAACCTGACT[CG]CAGGAGT[CG]TCCTC[
CG]TCTCTA[CG][CG]AAG[CG]CCCCCT[CG]AGGTGGGCAGG
GGGTGG[CG]G[CG]G[CG]A[CG]GTGG[CG]GAGC[CG]CAGA
G[CG]AGCTAGAGAG[CG]AGAG[CG]GTGAGACTCTG[CG]GA[
CG]TCTTCC[CG]CC[CG]C[CG][CG]CTC[CG]CCCCACCCAG
CCTC[CG]CAGCCT[CG]GGC[CG]GAT[CG]CC[CG]G[CG]GG
GAAGATG[CG]CCTCAAGAACCAGGTAGAAG[CG]CCT[CG]G[C
G]CAGGCCC[CG]CACCCC[CG]ACTCTGCC[CG]CCCTCTGTTA
[CG]GACACC[CG]CTGGGCCA[CG]TGGT[CG][CG]ACTGGCT
TCTCCCAG[CG]GCCAGCCTGGCCACCC[CG]ACTCCCAGGGAGG
GGGAGAGGGCCCTTGCAAGAGGATCCTGCCCC[CG][CG]CTGCC
CAGACCTGAGGGCTCCTGG[CG]AC
SEQ ID NO: 5249
G[CG]TTGGAGAGGGCTTTCA[CG]CC[CG]GGGTG[CG]GAAAA
GAAGCCAAATCCACCTTCCTCCACTGTTGGCCACCCTCCTGCAGG
CTCCCCCAG[CG]C[CG]ACTTCATTTTCTCTCTTCTCT[CG]CT
ACTTCCTCTCTGCCTTTCTTT[CG]TTATTTTTCCAC[CG]AGT[
CG]CCAC[CG]C[CG]CTTGCTTCCT[CG]GAGGAGCAA[CG][C
G]ACTGGCCAGCAAAGGGTGGCTCTGCTC[CG]AG[[CG]]CCAA
AGTTCTCCC[CG]CCTCAGC[CG]G[CG]CCTCCCTGTTGCTGAG
CCCTGG[CG]CCTGCCTGT[CG]CTTGGCTG[CG]CTAAA[[CG]
]CCCACTGTTGGTGAGATTCC[CG]AGTGGCACATCC[CG][CG]
CCCCTGGAAAGTCCTCTCCCCTCAAAGGCTGGG[CG]TCATAGTT
TGCAAAATTCCCTAAAACCTAAGAAGGAAAATAGGACTATGAGC[
CG]GTGAGAATTCTGAATTCAGAGT[CG]CACCTGAGGGATCTGA
AATT[CG]GTGGTTCACTGAAATAGGTTCAAAGAGGCTGTGACTT
AA[CG]AGCTTCTGGATGTGT[CG]CTTTTAAGGTTTTTCTAGCT
AAGGGTATTAAAGTAAGAATTATTTACACATTTTTATCTC
SEQ ID NO: 5250
AGAGGGCTTTCA[CG]CC[CG]GGGTG[CG]GAAAAGAAGCCAAA
TCCACCTTCCTCCACTGTTGGCCACCCTCCTGCAGGCTCCCCCAG
[CG]C[CG]ACTTCATTTTCTCTCTTCTCT[CG]CTACTTCCTCT
CTGCCTTTCTTT[CG]TTATTTTTCCAC[CG]AGT[CG]CCAC[C
G]C[CG]CTTGCTTCCT[CG]GAGGAGCAA[CG][CG]ACTGGCC
AGCAAAGGGTGGCTCTGCTC[CG]AG[[CG]]CCAAAGTTCTCCC
[CG]CCTCAGC[CG]G[CG]CCTCCCTGTTGCTGAGCCCTGG[CG
]CCTGCCTGT[CG]CTTGGCTG[[CG]]CTAAA[CG]CCCACTGT
TGGTGAGATTCC[CG]AGTGGCACATCC[CG][CG]CCCCTGGAA
AGTCCTCTCCCCTCAAAGGCTGGG[CG]TCATAGTTTGCAAAATT
CCCTAAAACCTAAGAAGGAAAATAGGACTATGAGC[CG]GTGAGA
ATTCTGAATTCAGAGT[CG]CACCTGAGGGATCTGAAATT[CG]G
TGGTTCACTGAAATAGGTTCAAAGAGGCTGTGACTTAA[[CG]]A
GCTTCTGGATGTGT[CG]CTTTTAAGGTTTTTCTAGCTAAGGGTA
TTAAAGTAAGAATTATTTACACATTTTTATCTCCTCAGCT
SEQ ID NO: 5251
CAGCAGAAGC[CG]CAGCTTCAGAGGCAGC[CG]GAGAGACCT[C
G]GAGCAGAGAAGG[CG]C[CG]C[CG]ACCCT[CG][CG]GCTG
CCTGGCC[CG][CG]GCTCCTACAAAGG[CG]GGCTAGC[CG]CC
[CG]CCCTCTCCCTTGCCTTCCTCCCCTTCTTTTCTGACTTTCCC
TCTTTCCCTTAAT[CG]CCTGCTTCTTCCTC[CG]GGTGGACTTA
[CG]GCCACCTTGCTCCTC[CG][CG]CTTCACCTCAT[CG]CCC
CCTCTTTCTTTCTTCTGCCTCTCTCTCTG[CG]CCCCCTTCTCTC
[CG]TGTCA[CG]CTCCCTCCTGGTTCTG[CG][CG]TCTACAAA
CTTTTGAGCAGAACA[CG]AGCCT[CG]GCAAA[CG]AGTCC[CG
]CAGCTCCTCCTGCTGCTCC[[CG]]CTGGTTCCTG[CG]GCTTC
TGCTCAGACACCAA[CG]CCAGA[CG]G[CG]ATGCCTCT[CG]G
GTGGTGACTCCAG[CG]CAGGAACTTGAAGAAG[CG]CTTTGCC[
CG]C[CG]TCCTACCTGGCAGCTCTCCTGGCAG[CG]GGAGGAGT
TGAAGGGTAAGGGAGGGAAAATCTTACCAAAG[CG]AC[CG]GCT
CACT[[CG]]ACTGCTGATTCTTT[CG]CTTGGCAT[CG][CG]T
CAGGGGAGTTAGCTTTCCT
SEQ ID NO: 5252
TCCTAGTCTGGGACATACTTGTACTAAACATATTTATATTAAAAA
CAATGTTTTAACATACTACTCTTGGACACTGTATAAAA[CG]GTT
G[[CG]]TGTCTGGAATGC[CG]T[CG]TTTTGCCC[[CG]][CG
]GCCCCAT[CG]TACATG[CG]CATCCC[CG]CAGC[CG]CCAC[
CG]TCTCCACTTTC[CG]CAGCAG[CG]TCCCTGA[CG]CTG[CG
][CG]ACC[CG]TTGCCAAGG[CG]ACTGGAAC[CG][CG]GCC[
CG]AGGTAACTGGAGGGCA[CG]G[CG]GGGG[[CG]]GGG[CG]
CTCATCCCAGG[CG]GCTTCTGATTGGCCT[CG]GC[CG]CCT[C
G]TG[CG]TCACAGCCC[CG]CCCCTGCC[CG]GGAGTCC[CG]C
AG[CG]GTGACTTGTCAC[CG]A[CG]CC[CG]G[CG]TGATCCA
G[CG]GACCAATGG[CG]GC[CG]CC[CG][CG]G[CG][CG]CA
GGGTG[CG]GGGATGGGGTC[CG]CC[CG][CG]G[CG]TTGGG[
CG][CG]CT[CG]CCAGCCCAGGGGT[CG]CCATGAC[CG]AGTG
GCCCAGGCC[CG]AG[CG]AAGCC[CG][CG][CG][CG]GTGAG
TC[CG]C[CG][CG]GCCCATC[CG]TCCCTC[CG]CC[CG]CCA
GAG[CG]TCCAT[CG]GGA[CG]CCCACC[CG]GGAGGGTCT[CG
]G[CG]C[CG][CG]T[CG]G[CG]GGAGCTGGCC[CG]GGA
SEQ ID NO: 5253
TTC[CG]TCAC[CG]GGGAAAGGAGGTACACACAGT[CG][CG][
CG][CG]CA[CG]GACACACACACACA[CG]CA[CG]CA[CG]CA
CACACAC[CG]GTGG[CG]AGTT[CG]AGCCC[CG][CG]GCCCC
TGTC[CG]CC[CG][CG]TG[CG]CCCCT[CG]ACACAGCT[CG]
CCTCC[CG]CCCCCTGATTCCTGCTGCTGC[CG]CCCAGAGGAGA
AAGGAACCTCTGCCT[CG]AATTTCCCCACTG[CG]C[CG]GG[C
G]CTG[CG]GAGAG[CG]G[CG]AGGGTGGG[CG][CG]AGG[CG
]GAGAA[CG][CG]ATGAATGAGTTCTCCCCT[CG]CCT[CG]GA
GTTGTCTGAGTTGG[CG]G[CG]CTG[CG]CCCAGGCTTC[CG]G
CTCTCAG[CG]CCCCA[CG][CG][CG][CG]TGGCTCCC[CG]G
GCTGCCACCCA[CG]CC[CG][CG]GC[CG]GGGC[CG]AGCCAG
CCA[CG]CAGGGCAGC[CG]AGGCTC[CG]GAGCTCCTGTCC[CG
]GCCCCAGTC[CG]GGTAAAAGGAGGGTTGTCCCCAG[CG]GAGG
[CG]CACAGC[CG][CG][CG]TTCTCCCTGCACTCTCTT[CG][
CG]GTCCCATCTGTTCCCCATGG[[CG]]TCTCATC[CG]CAAAC
C[CG]GATCCAGGCTTACCTGGAGAAGAACAAGAT[CG]GTCCCC
TGTTTGAGGTAAGG[CG]CT
SEQ ID NO: 5254
GAACCAGCCATGGTCC[[CG]]GGCTAATGCCCAGGGATAAGCAC
AGGGATAGGGATAGAGATAGGGATTGGGATTGGACTCCAGCTCCA
AGAGAAAGCACCT[CG]GGTCTATT[CG]TATTCTCCCCCTACCC
CCAAACAAAGCCACAGAAG[CG]ATCAG[CG][CG]CAGCTAAAG
GGTTTGGAGGTGAAAAGGAGGGCTGGGTGGGGAGGAGAGAAAGTG
AGAAGGGAGAGAAAAG[CG]G[CG]GCTGAGCAAAG[CG]GGAGC
[CG]G[CG][CG]GG[CG]GTGGGGCCTGACC[CG]GA[CG]C[C
G][CG]CACC[CG]GCTC[CG]CCC[CG][CG]CCCAG[CG]CCC
[CG]CTCACTCCCTGGC[CG][CG]CCTGCCTT[CG][CG]CCCC
TCTGTTTTCTCTCACTTACCAGCAGCTCTGCTGACAAG[CG]AGA
AAACTGACAAGTGAGAAAAGT[[CG]]TCTGTCAG[CG]CC[[CG
]]GGCTGGTGTCTGGGCTGTCAC[[CG]][CG]GG[CG][CG]GA
GGGAGGGG[CG][CG][CG]GGCAC[CG]C[CG]TCTGC[CG]GG
CACA[CG]CTGGGCTC[CG]ACCC[CG]GC[CG]GGCACACTCAC
CCC[CG]CAGGGCAGC[CG][CG]GCCACCAC[CG]ACCTG[CG]
GAGCTG[CG]TGGGCTTC[CG]GAACCCTCACCTCAA
SEQ ID NO: 5255
ACCAGCCATGGTCC[[CG]]GGCTAATGCCCAGGGATAAGCACAG
GGATAGGGATAGAGATAGGGATTGGGATTGGACTCCAGCTCCAAG
AGAAAGCACCT[CG]GGTCTATT[CG]TATTCTCCCCCTACCCCC
AAACAAAGCCACAGAAG[CG]ATCAG[CG][CG]CAGCTAAAGGG
TTTGGAGGTGAAAAGGAGGGCTGGGTGGGGAGGAGAGAAAGTGAG
AAGGGAGAGAAAAG[CG]G[CG]GCTGAGCAAAG[CG]GGAGC[C
G]G[CG][CG]GG[CG]GTGGGGCCTGACC[CG]GA[CG]C[CG]
[CG]CACC[CG]GCTC[CG]CCC[CG][CG]CCCAG[CG]CCC[C
G]CTCACTCCCTGGC[CG][CG]CCTGCCTT[CG][CG]CCCCTC
TGTTTTCTCTCACTTACCAGCAGCTCTGCTGACAAG[CG]AGAAA
ACTGACAAGTGAGAAAAGT[[CG]]TCTGTCAG[CG]CC[[CG]]
GGCTGGTGTCTGGGCTGTCAC[[CG]][CG]GG[CG][CG]GAGG
GAGGGG[CG][CG][CG]GGCAC[CG]C[CG]TCTGC[CG]GGCA
CA[CG]CTGGGCTC[CG]ACCC[CG]GC[CG]GGCACACTCACCC
C[CG]CAGGGCAGC[CG][CG]GCCACCAC[CG]ACCTG[CG]GA
GCTG[CG]TGGGCTTC[CG]GAACCCTCACCTCAACA
SEQ ID NO: 5256
GCCTG[CG]TCCAGACCCCCTCC[CG]GGTGGGCTCCTC[CG]AG
GACCTC[CG]CAGC[CG][CG]GG[CG]AGCTGGGTGCCTG[CG]
AGT[CG]C[CG]C[CG]CAGTCTGAGCCTCTGGTGCCCCTTAG[C
G]TTGGCTCT[CG]GAGTTCTGAGAAGGGAAAGGAGACAGACACT
CTGGGAAAATCTGGGGTCACCT[CG]ATTC[[CG]]GCTCCTA[C
G]CTGGATGC[CG]TGGGCAAGCAT[CG]GTCTCCTTTGATCTGA
AGATCCTGCCCACTC[CG]CCCCTTGCCCCTGCCAGACC[CG][C
G]GGAGCAGCAGGTGG[CG]TGGGTGCCAGA[[CG]]CTGC[CG]
GTG[CG]CAC[CG]AGTGCAG[CG]CTGCCC[CG]GCT[CG]CCC
[CG]GGGGT[CG][CG]TGGGACAG[CG]GTTTG[CG]GTGT[CG
]CCCTCTCTG[CG]GTGCTTTCCAAGGACCCTCATGCCC[CG]CC
[[CG]]GGTGCACATTCACCACATGGGTCTTACTAGGTGACCTCT
TTCTAAGTAAAATGCATTGAGAAACCAAAGGGATGGGGCATC[CG
]TATCCATTTTCCTTTAAGGAG[CG]CTGTGGGTGTTGGGGATG[
CG]GGAGAAGTAGGTGTTTTTTAAAGAGAAAATTGCAAAACCTCT
TGGGCAGGATT
SEQ ID NO: 5257
CTCT[CG]GAGTTCTGAGAAGGGAAAGGAGACAGACACTCTGGGA
AAATCTGGGGTCACCT[CG]ATTC[CG]GCTCCTA[CG]CTGGAT
GC[CG]TGGGCAAGCAT[CG]GTCTCCTTTGATCTGAAGATCCTG
CCCACTC[CG]CCCCTTGCCCCTGCCAGACC[CG][CG]GGAGCA
GCAGGTGG[CG]TGGGTGCCAGA[CG]CTGC[CG]GTG[CG]CAC
[CG]AGTGCAG[CG]CTGCCC[CG]GCT[CG]CCC[CG]GGGGT[
CG][CG]TGGGACAG[CG]GTTTG[CG]GTGT[CG]CCCTCTCTG
[CG]GTGCTTTCCAAGGACCCTCATGCCC[CG]CC[CG]GGTGCA
CATTCACCACATGGGTCTTACTAGGTGACCTCTTTCTAAGTAAAA
TGCATTGAGAAACCAAAGGGATGGGGCATC[CG]TATCCATTTTC
CTTTAAGGAG[CG]CTGTGGGTGTTGGGGATG[CG]GGAGAAGTA
GGTGTTTTTTAAAGAGAAAATTGCAAAACCTCTTGGGCAGGATT[
CG]ACCTACTAGCCTCTTCTGCTGCATAATAAGGTACTGTATACA
CTAAATTTTACCCCTAAAAATTTTCAGTTCTTACCAACTATAGGT
AATGATCATCCAAAAGC[CG][CG]TCCTGTTTG
SEQ ID NO: 5258
T[CG]CATGGC[CG]AGGA[CG]CTTT[CG]CTG[CG]AGTTTGG
GGT[CG]AGGGGCAGTTCCCTCC[CG]GAGTCATTAACTT[CG]T
CTCCT[CG]TGCTCAGGGACCAGCAACTTGGTGCTG[CG]GG[CG
]CCAGAAAGAAAAGGGTAG[CG]CCT[CG][CG]GAG[CG][CG]
[CG]GGGGAAGAGGGACTG[CG]GCAG[CG]GGA[CG][CG]AGG
G[CG]GGAGGGG[CG]CAGCAG[CG]GGGAGC[CG]GAG[CG]CA
G[CG]GAGGGGAG[CG]CTGGGGGG[CG]GGGAG[CG]CT[CG]G
GCAGCCACCCTGTCCC[CG]TCTGGAGCCC[CG][CG]CTG[CG]
TCTAGGAGGG[CG]CAACA[CG]CAGTCCC[CG][CG]GGGGCCA
GAGCT[CG]GAGCCCCCTAGTGCATGCCCCCTCTCCT[CG][CG]
CCCTGC[CG]AGGCCT[CG]GCCCTGCCTGC[CG]TGCCCTGGGC
CTGGTTGTGCC[CG]GGGGTCCC[CG][CG]GGCAGGG[CG][CG
]GGGCAGGCAGGG[CG][CG][CG][CG]C[CG]A[CG]CTCTTT
TGTCTGATAACTAATTTGAGTTAATG[CG]ATCTTTATGTAAAGC
TAACAG[CG]GATAATTGTCTATTTTCT[CG]CCAACAGTCTCCA
TCACAATCACTTATCTGGAAACCTG[CG]GTTGGATTAAT[CG]T
TA
SEQ ID NO: 5259
TGTGGGGGGCAGG[CG]CTCAGGACCCCAATTCCCTCCAAGTTGG
GC[[CG]][CG]GTGGGGG[CG]GGTGGAGGTGG[CG]C[CG]GG
CAGCTGG[CG]GC[CG]CT[CG]CTGGGGCTGGGTTTGTGC[CG]
AG[CG][CG]AGTCTGGAAGGAG[CG]AGC[CG]CAGC[CG][CG
][CG]GGGGTGGAGC[CG]AACTGAAGTCACTGGCTGAGCAGGCA
[CG]GG[CG]CTGG[CG]GGCA[CG][CG]GGAAGGAGGG[CG]G
C[CG]ATGCCAGGTCTC[CG]GG[CG]CTGGAGG[CG]C[CG]C[
CG]G[CG]ACCCCCCCCTAGGCCT[CG]CAGG[CG]CTGCCTC[C
G]CCTATCCC[[CG]]CTGACCACTCTG[CG]CCCC[CG]CAGC[
CG][CG]GGTCTCCCTGCCTAGTC[CG][CG]CACCCTGCC[CG]
[CG]GGGG[CG]GT[CG]ACC[CG]GGCTCCTGGAGGGTGGATAT
GT[CG]GAGTTGGGAAGGGGCTTTTCTTTGCCATCTGATTCCCAA
TGACTGGACACCTCTACCTGGCTTTTCACTCAGGCTC[CG]TGTT
CTGGCCTGTGGGAAACCCTAGAAAGGTAGCCCTCAAAAATAATGT
TGGATCCTTTGGGCCTATGTACACACAACATGCTCAAAAGATGAT
CTGAGGCAACAATTATCACTCTCTA
SEQ ID NO: 5260
GAAACCCCATTGCAGTTCCAG[[CG]][CG]GCTTCTTCCCAGAG
CAGC[CG]C[CG]C[CG]C[CG]C[CG][CG]CTCCTCACACCTG
CATTGCCAGCAGCAGCAACAGAGCCAGGACAAGC[[CG]]TGCC[
CG]CCCTT[CG][CG]CCCCTCC[CG]CACCCTCACCACCACC[C
G]CACCT[CG][CG]CACCAGCAGC[CG]GCCAG[CG]G[CG]GC
AGCAGCCCATGCCTC[CG]GTGCAACAGCTG[CG]CCTCCTC[CG
]GTGCCC[CG]G[CG]G[CG]GGGG[CG]GGAGATAACCTGTCCC
TGCTGCTC[CG]CACCTCCT[CG]CC[CG]G[CG]G[CG]CCTTC
[CG]GACC[CG]CACCTCCT[CG]C[CG]CTGT[CG]GGCT[CG]
TCCTGCTGCTGCTGCTGCTGCT[CG]T[CG][CG]C[CG]GGGCA
GCCAGCTCAATGTGAG[CG]AGCTGA[CG]C[CG]TCCAGCCATG
CCAGTG[CG]CTC[CG]GCAGCAGTA[CG][CG]CAGCAGTC[CG
][CG]CAGCAGT[CG]G[CG]TC[CG]CCTCCCAGTACCACCAGT
GCCACAGCCTGCAGCC[CG]C[CG]CCAGCCCCA[CG]GGCAGCC
T[CG]GCAGTCTGGGCTC[CG]GGCCCC[CG]CTCT[CG]CACCA
CCACCACCACC[CG]CACC[CG]G[CG]CACCACCAGCACCACCA
GCCCCAGG
SEQ ID NO: 5261
GG[CG]GGGCACAGGA[CG]GTAGTCTAAGAGTGTT[CG]CATGG
C[CG]AGGA[CG]CTTT[CG]CTG[CG]AGTTTGGGGT[CG]AGG
GGCAGTTCCCTCC[CG]GAGTCATTAACTT[CG]TCTCCT[CG]T
GCTCAGGGACCAGCAACTTGGTGCTG[CG]GG[CG]CCAGAAAGA
AAAGGGTAG[CG]CCT[CG][CG]GAG[CG][CG][CG]GGGGAA
GAGGGACTG[CG]GCAG[CG]GGA[CG][CG]AGGG[CG]GGAGG
GG[CG]CAGCAG[CG]GGGAGC[CG]GAG[CG]CAG[CG]GAGGG
GAG[CG]CTGGGGGG[CG]GGGAG[CG]CT[CG]GGCAGCCACCC
TGTCCC[CG]TCTGGAGCCC[CG][CG]CTG[CG]TCTAGGAGGG
[[CG]]CAACA[CG]CAGTCCC[CG][CG]GGGGCCAGAGCT[CG
]GAGCCCCCTAGTGCATGCCCCCTCTCCT[CG][CG]CCCTGC[C
G]AGGCCT[CG]GCCCTGCCTGC[CG]TGCCCTGGGCCTGGTTGT
GCC[CG]GGGGTCCC[CG][CG]GGCAGGG[CG][CG]GGGCAGG
CAGGG[CG][CG][CG][CG]C[CG]A[CG]CTCTTTTGTCTGAT
AACTAATTTGAGTTAATG[CG]ATCTTTATGTAAAGCTAACAG[C
G]GATAATTGTCTATTTTCT[CG]CCAACAGTCTCCATCACAATC
ACTT
SEQ ID NO: 5262
ACTGATCAGCTCCAAGGACAGAC[CG]CTGGGAAGGGAAAAA[CG
]GCCCACAT[CG]AACTGGATGC[CG]GATGGAAACCTCTCTG[C
G]CTATTAGACTG[CG]TCCAGTACAGCAGATGGCA[CG]AGCA[
CG]TG[CG]G[CG]CTCAGCTTAGGCTCT[CG]GAGGCAGCTGAG
TTGGAAATCC[[CG]]A[CG]GAAAGCACCCACAAGCTCCCACTC
TG[CG]CTGGCCCACC[CG][CG]TGCA[CG]CCCACCCCCCA[C
G][CG][CG]TCCCTGGCTCAGAAG[CG]CACAGATGTTTACTGC
TTAGAGC[CG]GTAC[CG]CTGGGGAGAT[CG]AG[[CG]]ACTT
G[CG][CG]G[CG]CACAGTG[CG]G[CG]CTGGCAGGGCTCTGG
GCTCC[CG]GT[CG]GGGGTT[CG]AG[CG]GCCAAGGGATGGGG
GTGGGGG[CG]GGGAGAGTGGGGGGAGGG[CG]AAAGAC[CG]C[
CG]AGAGGAGGGGGGAGTGGGTGGACTAATGATGAAAAAGTCTCC
TCCATCCCAGTTCCTTAATTAAATGCATGGAAAGAAC[CG]AGG[
CG]AGCACATCTGGTTTCAATCTACAGCCCTTTGATGGCATCAAA
TGTTCTTTTCCCAGATCAGGGCTGGAAGTTCTGGGCTAACTATGG
C[CG]TTTGGA
SEQ ID NO: 5263
G[CG]AGCTGTTGGCTG[CG]AGGTGGG[CG]TG[CG]TGTGCTC
CATGAG[CG][CG]CCCCT[CG]CTCCCCTTGC[CG]CTT[CG][
CG]CC[CG]GCCTGCCAGCC[CG]ACTCTGGT[CG]C[CG]GCT[
CG]GTGGTGG[CG]G[CG]G[CG]GAGGCAG[CG]GCAGCTGGAG
CAGCAG[CG]CCTCTCTCCC[CG]CAGGACTGGCTCCTACCCTTG
GC[CG]TGATCCCCTCTGCT[CG]CTC[CG]CACCTGCC[CG]CC
TGGAGTCTTGGCTCAGCCCTCC[CG]CAGGG[CG][CG]GGG[CG
]CAGGGTG[CG]GGG[CG][CG]CTG[CG][CG]GGGCCT[CG]G
GAGGGGCTCC[[CG]]GAGCC[CG]TCTGTG[CG]CCTCCCACCT
[CG]GACC[CG]CTCCACTCC[CG]CCTCAAAGC[CG]GGGGGCT
GGGA[CG]C[CG]CTCCC[CG]CAGCCAATCAG[CG]GCTGCCTG
GAAG[CG]GAGTGTGTGTGGGGTGGGAGGGATG[CG]GAAGAGGA
GGGGGTCCCAGC[CG]CCT[CG]CTTGGCTCCACTTTCAGGGGAG
AAGATGCAGTTAGACCTGGGAGCCC[CG]AAGGAAGAC[CG]GAG
TGGAAAACAGGCTATCCCCAATCTCCAACCC[CG]AGGGAGGGG[
CG]TGGTGTGGGTAGGATCTGCCTCTCC[CG]CAG
SEQ ID NO: 5264
[CG]TGACTG[CG]GAGCCTGGAGGATG[CG]GGCTCCC[CG]AG
[CG]GGCTTCCCCTCTGCC[CG]G[CG][CG]ATGGAG[CG]GGT
[CG]GTGAG[CG]GAACAAAGG[CG]CCTGCC[CG]GGGGAG[CG
]GGTCTTC[CG]CTGAAGAG[CG][CG]GGTGG[CG][CG]C[CG
][CG]A[CG]GC[CG]CCTG[CG]GGCCAAGGTGGGAGGCTGTG[
CG]GCCCCAGG[CG][CG]G[CG][CG]CTC[CG]C[CG]TGGG[
CG]GG[[CG]]G[[CG]]ATCCCTGAGTC[CG]GCACCTGTG[CG
]C[CG]CTCCCTCTGTGCCTCC[CG]GGCAGCCC[CG]CCTGC[C
G]GCCT[CG]GAGTC[CG][CG]G[CG]C[CG]G[[CG]]GCTAG
AGGTCCAGAGG[[CG]]AACCACTTGCTGGTGCAGAAGAGAAACC
CTCAAATCCCTGGCCTTT[CG][CG]GAGA[CG]CCTGGA[CG]G
GGC[CG]TG[CG]C[CG]TGGACTGAGCAGG[CG]TCT[CG]GGG
AGCACTTCTGCAGAG[CG]AGGACTTCCATGTGAG[CG]ATTC[[
CG]]TTCTCCCCACCACCAATC[CG]ACCTCCCAGC[CG]TCTC[
CG]C[CG]CC[CG]AGCATCCTTGAGGTGGGA[CG]AGCAGGGGC
TTGGATCCCTGC[CG]GC[CG]TCTGGTGTGTGAGGCTTGCA[[C
G]]GCCCCTGGCTGCCC[CG][CG]CCT[CG]CC
SEQ ID NO: 5265
TGTCCCACT[CG]GGCCC[CG][CG]CCCCCACTTAGC[CG]CTG
AAGG[CG]TCT[CG][CG]GTGACTTTACCTTGCCTGGGGTGGAA
GTCAACTTCCCAAGACCTGGTGATGAAGAG[[CG]]AGAGATGGT
AGAGCAGCCCAGTCAGCCACTTGGCCACCCACATGGTTCCACCCT
GGGGACCC[CG]AT[CG]GGGAGGCCCACCAGAGC[CG]C[CG]G
CAGCCAAGC[CG]GCTTCCCT[CG][CG]CTC[CG]ATCCCTCTC
CTCCCTCT[CG]CC[CG]C[CG]CTCTC[CG][CG]CCTCAGCAG
CCTAGGGAACAGCAG[CG][CG]GGCCCTGGGC[[CG]]GCTGGT
CCAGCCACAC[CG]CCTGTCTC[CG]CT[CG]CTGAGC[CG]CTG
CTTCAT[CG]CACTGCAGCCTCACCCCCCTTCCTG[CG]CC[CG]
[CG]CTGCCCTCAGAAACTCTCTGCTCAGGTTCAGCT[CG]G[CG
][[CG]]GGGAAGCAACT[CG]ACCTAGCAGGAATGGTGCCAATA
TAGCCACCC[CG]GAC[CG]G[CG]AG[CG]C[CG][CG]GAGAG
CC[CG]CCCCTATGGGCTGTTCC[CG]GCCAATCAG[CG]GGAGG
[CG][CG]GCCCCTGCT[CG]CTTTTCCC[CG][CG]CAGCCCCC
TCCCTTCTCCACTCAGC[CG]CAAGGGGACTGC
SEQ ID NO: 5266
[CG]CC[CG]CCTGCCTGTCTCTAGGCTG[CG]GGAGGATTAGAC
ACATG[CG]CA[CG]ATTGGTCTCTTTGTGTGCACCAGGAAGGGT
CTGG[CG]T[CG]ATTTACACTTCCTAA[CG]GGGCAGAGACCTG
GAA[CG]GC[CG][[CG]]AGTGCTTGGGAATAAAAATGTACTGT
AAACAAGGCCAATGAAATAAGTGTCT[CG]CTCC[CG]CTGGCCT
TC[CG][CG]TTGGAAGTGTCCCCACTGCCCTTTCCC[CG]CTGC
ACAGCTTG[CG]AGTGGAGATTTAGAGCC[CG]GGAATGAGG[CG
]C[CG]TGTCCTTAGGAATCT[CG][CG]CC[CG]GGGAG[CG]C
TGAGGGAC[CG]GGGAGC[CG]GGTGGGGAG[CG]GGCCT[CG]G
GG[CG]CC[CG]G[CG]G[CG]GAGG[CG]CTCCCACC[CG][CG
][CG]GAGAGGGATGGAG[CG][CG]TCTTG[CG]AG[CG]TCTC
[CG]AGCCCAGCCACCCCA[CG]GGCCAGGGGGAGGGAG[CG]GG
GC[CG]G[CG]CATCAGCC[CG]CCCCCCC[CG]GCC[CG]CCCC
CTCCCCC[CG][CG]C[CG]GGGAGC[CG][CG]GTGGTG[CG]C
TC[CG]GCCAGCTGTG[CG]C[CG]C[CG]AG[CG]AGG[CG]CC
AGCC[CG]TGGGTGCTGC[CG]GGGG[CG]G[CG]G[CG][CG]G
TGGGGGCTCC[CG]CA
SEQ ID NO: 5267
AGGATG[CG]GGCTCCC[CG]AG[CG]GGCTTCCCCTCTGCC[CG
]G[CG][CG]ATGGAG[CG]GGT[CG]GTGAG[CG]GAACAAAGG
[CG]CCTGCC[CG]GGGGAG[CG]GGTCTTC[CG]CTGAAGAG[C
G][CG]GGTGG[CG][CG]C[CG][CG]A[CG]GC[CG]CCTG[C
G]GGCCAAGGTGGGAGGCTGTG[CG]GCCCCAGG[CG][CG]G[C
G][CG]CTC[CG]C[CG]TGGG[CG]GG[CG]G[[CG]]ATCCCT
GAGTC[CG]GCACCTGTG[CG]C[CG]CTCCCTCTGTGCCTCC[C
G]GGCAGCCC[CG]CCTGC[CG]GCCT[CG]GAGTC[CG][CG]G
[CG]C[CG]G[CG]GCTAGAGGTCCAGAGG[[CG]]AACCACTTG
CTGGTGCAGAAGAGAAACCCTCAAATCCCTGGCCTTT[CG][CG]
GAGA[CG]CCTGGA[CG]GGGC[CG]TG[CG]C[CG]TGGACTGA
GCAGG[CG]TCT[CG]GGGAGCACTTCTGCAGAG[CG]AGGACTT
CCATGTGAG[CG]ATTC[[CG]]TTCTCCCCACCACCAATC[CG]
ACCTCCCAGC[CG]TCTC[CG]C[CG]CC[CG]AGCATCCTTGAG
GTGGGA[CG]AGCAGGGGCTTGGATCCCTGC[CG]GC[CG]TCTG
GTGTGTGAGGCTTGCA[[CG]]GCCCCTGGCTGCCC[CG][CG]C
CT[CG]C[CG]GAGCC[CG]AGGGGG[CG]CA
SEQ ID NO: 5268
ATTAACCACTTTGCCAAAGGCCTGGC[CG]GGGGGGGGGAGAGGG
GACAAGGT[CG]TCAGGCCTGGCAGTCCAGGTGGGCACCCTGGGT
GGTGGCAGGACCCTAGAGCCCCCAGATCC[CG]GCTACTCAGGAT
CAACAGCAGCAGGTTCCAGGGAGGAAGGGTACTCACCTGGCTCCA
CTGGGCTCTGCAGAGGAAAAGCT[CG]CAGGGAGGACAGGGAGAG
ACCTCTG[CG]TGGATGAGCTCCC[CG]GCAGGGGACAGCAGC[[
CG]]GA[CG]CCTGGGTCA[CG]GGCAGTGG[CG]TG[CG]CTTG
CTCCTCTCC[[CG]]GCTGCCAAGGAGGCTGCTTCCATGCCC[CG
]CAGCCAGCTCCTGTCTGGCTGTGCAG[CG][CG]T[CG]CTGCT
GCCCCTGGTGAGA[CG]ATGTTCCCAGCTGAGCAG[CG]CAAGAA
GAGAGGCAAGAG[CG]AAATCAAAGCAG[CG]TGGAGGAGAGAGA
GGCAGAGAAGGGAGAAAGAA[[CG]]AGAGAGCCAGAC[CG]AAT
CTGCCTCCACCAATGGGATCAG[CG]GCTG[CG]CAG[CG]TCAG
GTGATAATAAGCAGGCTTGGTCCCTGCT[[CG]]C[CG]GGCTGG
TGGGAGGGGGAAAGGGCAGAGGGTCTCCCAGG
SEQ ID NO: 5269
AGACTGAG[CG]G[CG]AGAAAGTG[CG]AGC[CG]GGC[CG]GC
AGAATCTGCCTGG[CG]GG[CG]CTGGAGCCTG[CG]TTACT[CG
][CG]GCC[CG]CAGC[CG]TC[CG]GCTACTTTG[CG]TTTGGC
C[CG]GCCAG[CG]CC[CG]GG[CG][CG]C[CG][CG]CCATTG
CCTGCAGGCTAGGACTT[CG][CG]AGGTGGGT[CG]ACTCCCCC
TCCCTCCTCCTCTTCTTCCTCCTCTTCCTCCTCCTCTTGTTCCTC
CTCCTCCTCC[CG]ATTTTCCCTCCT[CG]GCTGG[CG]AGGGTG
GGGGGGG[CG]GGGGAGGC[CG]GGGCT[CG]CCC[CG]AGCAGC
CA[[CG]]ATGCTCCTGGA[CG]C[CG]GCCCCCAGTACCCAG[C
G]AT[CG]G[CG]TGACCACCTTTGG[CG][CG]TCC[CG]CCAC
CACTC[CG][CG]GG[CG]A[CG]TGGC[CG]AA[CG]AGA[CG]
TGGGCCTGGGCATCAACC[CG]TT[CG]C[CG]A[CG]GCATGGG
[CG]CCTTCAAGCTCAACCCCAGTT[CG]CA[CG]AGCTGGCTT[
CG]GC[CG]GCCAGA[CG]GCCTTCA[CG]T[CG]CAGG[CG]CC
AGGCTA[CG][CG]GCTGCTG[CG]GCCCTGGGCCATCACCATCA
CC[CG]GGCCA[CG]T[CG]GCTCCTATTCCAG[CG]CAGCCTTC
AACTCCA[CG][CG]GGACT
SEQ ID NO: 5270
GCATTTCAGGAAGTCATTGGTATCCATTGATAACAACCA[CG]TT
CTCCATTTTCCCAGAAGAGCCAGGAGCCACTTTCAGTCCTAGTTA
A[CG]AAAA[CG]CATGCATAAATCAACCACT[CG]GGT[CG]GT
[CG]GTCTTCTCCACTCCTTCAGCAGAGGA[CG]T[CG]G[CG]C
C[CG]GCTGCCT[CG]CC[CG]CCTGCCTGTCTCTAGGCTG[CG]
GGAGGATTAGACACATG[CG]CA[CG]ATTGGTCTCTTTGTGTGC
ACCAGGAAGGGTCTGG[CG]T[CG]ATTTACACTTCCTAA[CG]G
GGCAGAGACCTGGAA[CG]GC[CG][[CG]]AGTGCTTGGGAATA
AAAATGTACTGTAAACAAGGCCAATGAAATAAGTGTCT[CG]CTC
C[CG]CTGGCCTTC[CG][CG]TTGGAAGTGTCCCCACTGCCCTT
TCCC[CG]CTGCACAGCTTG[CG]AGTGGAGATTTAGAGCC[CG]
GGAATGAGG[CG]C[CG]TGTCCTTAGGAATCT[CG][CG]CC[C
G]GGGAG[CG]CTGAGGGAC[CG]GGGAGC[CG]GGTGGGGAG[C
G]GGCCT[CG]GGG[CG]CC[CG]G[CG]G[CG]GAGG[CG]CTC
CCACC[CG][CG][CG]GAGAGGGATGGAG[CG][CG]TCTTG[C
G]AG[CG]TCTC[CG]AGCCCAGCCACCC
SEQ ID NO: 5271
CAGAGGA[CG]T[CG]G[CG]CC[CG]GCTGCCT[CG]CC[CG]C
CTGCCTGTCTCTAGGCTG[CG]GGAGGATTAGACACATG[CG]CA
[CG]ATTGGTCTCTTTGTGTGCACCAGGAAGGGTCTGG[CG]T[C
G]ATTTACACTTCCTAA[CG]GGGCAGAGACCTGGAA[CG]GC[C
G][[CG]]AGTGCTTGGGAATAAAAATGTACTGTAAACAAGGCCA
ATGAAATAAGTGTCT[CG]CTCC[CG]CTGGCCTTC[CG][CG]T
TGGAAGTGTCCCCACTGCCCTTTCCC[CG]CTGCACAGCTTG[CG
]AGTGGAGATTTAGAGCC[CG]GGAATGAGG[[CG]]C[CG]TGT
CCTTAGGAATCT[CG][CG]CC[CG]GGGAG[CG]CTGAGGGAC[
CG]GGGAGC[CG]GGTGGGGAG[CG]GGCCT[CG]GGG[CG]CC[
CG]G[CG]G[CG]GAGG[CG]CTCCCACC[CG][CG][CG]GAGA
GGGATGGAG[CG][CG]TCTTG[CG]AG[CG]TCTC[CG]AGCCC
AGCCACCCCA[CG]GGCCAGGGGGAGGGAG[CG]GGGC[CG]G[C
G]CATCAGCC[CG]CCCCCCC[CG]GCC[CG]CCCCCTCCCCC[C
G][CG]C[CG]GGGAGC[CG][CG]GTGGTG[CG]CTC[CG]GCC
AGCTGTG[CG]C[CG]C[CG]AG[CG]AGG[CG]CCAGCC[CG]T
GGGTGCTGC[CG]GGG
SEQ ID NO: 5272
AAGA[CG]AAC[CG]AGTGC[CG]CTG[CG]GGAAGACACTGAAG
CAAGAG[CG]ACACCCCTTTC[CG]CCCCCCACCTTGCAG[CG]C
AGGCTTTGAAAGCTGCTCCCCCACCTGAATGGAAACT[CG]GAAC
[CG]C[CG]GG[[CG]]G[CG][CG]TTCCTTCT[CG]CCCAGCC
TTGCAATCCATGC[CG]AGAGGAAGGCAGTG[CG]AGCC[CG][C
G]CCAG[CG]CCCAGCTCC[[CG]]GGACAGGGT[CG]GCAATGC
TGCTGAGCAGAACTTGAT[CG][CG]CTCCTTCCT[CG]CTGCTA
GTGGAAG[CG]ATGCTGCA[CG]GCACAGCTAG[CG]CTTCCC[[
CG]]GCTCTCCTTCAAGCTGAAGGTTAC[CG]ACC[CG][CG]CA
GCCAGCCCCAGCACTGTGAGCTG[CG][CG]CCTCAGGTC[CG]G
GCTC[CG]GCTGCTTGG[CG]G[CG]G[CG]CCCAGGGCAACAAC
[CG]GGC[CG]CC[CG][CG]C[CG]GGG[CG]CACTGCACCAG[
CG]GCTT[CG]GCTTGGTGGATGTGTATGCATGAGTTCTGCAC[C
G]AATGGG[CG]CAAAAAG[CG]CCCCAGC[CG]GTCCACC[CG]
CTCCT[CG]ATCTTCCAGATCAGCAAGCCCC[CG]CTGCAGAG[C
G]GAGATTGGGAG[CG]CAGGGGCAG[CG]GCTC[CG]AGAG[CG
]C
SEQ ID NO: 5273
GGAGGGGAAAGA[CG]AAC[CG]AGTGC[CG]CTG[CG]GGAAGA
CACTGAAGCAAGAG[CG]ACACCCCTTTC[CG]CCCCCCACCTTG
CAG[[CG]]CAGGCTTTGAAAGCTGCTCCCCCACCTGAATGGAAA
CT[CG]GAAC[CG]C[CG]GG[CG]G[CG][CG]TTCCTTCT[CG
]CCCAGCCTTGCAATCCATGC[CG]AGAGGAAGGCAGTG[CG]AG
CC[CG][CG]CCAG[CG]CCCAGCTCC[[CG]]GGACAGGGT[CG
]GCAATGCTGCTGAGCAGAACTTGAT[CG][CG]CTCCTTCCT[C
G]CTGCTAGTGGAAG[CG]ATGCTGCA[CG]GCACAGCTAG[[CG
]]CTTCCC[CG]GCTCTCCTTCAAGCTGAAGGTTAC[CG]ACC[C
G][CG]CAGCCAGCCCCAGCACTGTGAGCTG[CG][CG]CCTCAG
GTC[CG]GGCTC[CG]GCTGCTTGG[CG]G[CG]G[CG]CCCAGG
GCAACAAC[CG]GGC[CG]CC[CG][CG]C[CG]GGG[CG]CACT
GCACCAG[CG]GCTT[CG]GCTTGGTGGATGTGTATGCATGAGTT
CTGCAC[CG]AATGGG[CG]CAAAAAG[CG]CCCCAGC[[CG]]G
TCCACC[CG]CTCCT[CG]ATCTTCCAGATCAGCAAGCCCC[CG]
CTGCAGAG[CG]GAGATTGGGAG[CG]CAGGGGCAG[CG]GCTCC
SEQ ID NO: 5274
GGGTGGC[CG]GGCAGCACCAGGGACAG[CG]CC[CG]GGACTCC
ACTGGGGAC[CG]GCTCCTGGGCTTCCCAG[CG]T[CG][CG]GG
TAGAGGTACAGCTGCTC[CG]TGTGC[CG]CAGGCTCCAGATTCT
[CG]CCACCCCACCCCTCCCTCAGAAACT[CG]GACTGCTCT[CG
]TCTGC[CG]TGTGGTTCTCTTTTCTTC[CG]AAAGGCCAGTGTC
TTATCTCTCCACTTCAAGTCCAGAGGACTTGCTCAGTCTCCTCCC
CTTAAGTCATTTCCACCATCCTCAGGCAGCTGTGGGAAGC[CG]A
GAGTCCTGGACTGTT[[CG]]TC[CG]GGTGCCAG[CG]CTGGCA
GTCCCAGTC[CG]TC[CG]GTGCAGCAGCC[CG]G[CG]CATTCC
CCTCTCTCCCTCCCTCTTGCTCTCCCTCCCTTTCTGTCTTCCTCT
CTTTCCTCCTCTACTGCTCCCTCCCTCTCTTGCCTCTTAAGTTTC
CTGCAC[CG]TGAATCCAACTGTGCCAAGCCTTGGCTCC[[CG]]
[[CG]]AACCAATCCTGAG[CG][CG]ACC[CG]GGCACTGGGA[
CG]G[CG]ACTC[CG]CCAAAGCTGGA[CG]AGGCAGC[CG]GAC
C[CG]TCTG[CG]CT[CG]AGCATGGAGA[CG]GAG[CG]CCTGG
GAGGGCA
SEQ ID NO: 5275
GA[CG]AC[CG]GACAGAGAGAGGCACTGAC[CG]AT[CG]CCAG
CAGCCTCC[CG]GTGGGAC[CG][CG]TCTCCTGCACACCC[CG]
[CG]CAG[CG]CCCCC[CG]C[CG]GAGC[CG]CAC[CG]GGCAA
GC[CG]G[CG]AGGGAG[CG]GGGCTGATTGG[CG]GC[CG]C[C
G]G[CG]GCCAGGGGAGGGGG[CG]C[CG][CG][CG]GGGCCAT
GGCAGGCT[CG]GAGG[CG]TCCTAGCC[CG]AGC[CG]GAGC[C
G]ATC[CG]AGCCCA[CG][CG]GC[CG]C[CG]CCTCTC[CG]C
TCC[CG]GGCCCC[CG]C[CG]CCAC[CG][CG]CCCCC[CG][C
G]GGAGATGGAACAG[CG]GAAC[CG]GCT[[CG]]GTGCCCT[C
G]GATACCTGC[CG]CCTCTGCTGCTGCATGCCCTGCTGCTCTT[
CG]TGGC[CG]A[CG]GTGAG[CG][CG]GGAACTTTGCTGC[CG
]CTGTGGACT[CG]GGGGGGTCTTTGCTGGGGC[CG]CCAC[CG]
AGAAGAAGAAGAAAGAGAA[CG]AAC[CG]GGAAGCTT[CG][CG
]GTCTCCAGCAATCC[CG]CTGTGCAGCCTGGGCC[CG]GAGGGG
TAGGGAGAGG[CG]AG[CG]G[CG]AAGGGT[CG]C[CG]CAGCA
GCAG[CG]C[CG][CG]CC[CG]AGT[CG]TTCCCAGTCC[CG]C
[CG]GGGCCC[CG]GCTG[CG]GAAAGGATGCCTGC
SEQ ID NO: 5276
GCCAGTGTCTTATCTCTCCACTTCAAGTCCAGAGGACTTGCTCAG
TCTCCTCCCCTTAAGTCATTTCCACCATCCTCAGGCAGCTGTGGG
AAGC[CG]AGAGTCCTGGACTGTT[CG]TC[CG]GGTGCCAG[CG
]CTGGCAGTCCCAGTC[CG]TC[CG]GTGCAGCAGCC[CG]G[CG
]CATTCCCCTCTCTCCCTCCCTCTTGCTCTCCCTCCCTTTCTGTC
TTCCTCTCTTTCCTCCTCTACTGCTCCCTCCCTCTCTTGCCTCTT
AAGTTTCCTGCAC[[CG]]TGAATCCAACTGTGCCAAGCCTTGGC
TCC[CG][CG]AACCAATCCTGAG[CG][CG]ACC[CG]GGCACT
GGGA[CG]G[CG]ACTC[CG]CCAAAGCTGGA[CG]AGGCAGC[C
G]GACC[CG]TCTG[CG]CT[CG]AGCATGGAGA[CG]GAG[CG]
CCTGGGAGGGCA[CG]TC[CG]GGG[CG]CTGGAGA[CG]CCAGG
CC[CG]AGTAGCTTCTCCATGGAGCCTGCCCAGAG[CG]GTCCCT
TCT[CG]CAGGATT[CG]CCCCAAGTCCTGTG[CG]GTA[CG]TA
CCCTTTGCCTC[CG]CTGTTCC[CG]GAGGGTACACCTGGGAG[C
G]GGGTGGTGGGCA[CG]AGGAAGGGA[CG]GCAGGGAGGGG[CG
]CTGGTT
SEQ ID NO: 5277
AGGCCAGTGTCTTATCTCTCCACTTCAAGTCCAGAGGACTTGCTC
AGTCTCCTCCCCTTAAGTCATTTCCACCATCCTCAGGCAGCTGTG
GGAAGC[CG]AGAGTCCTGGACTGTT[[CG]]TC[CG]GGTGCCA
G[CG]CTGGCAGTCCCAGTC[CG]TC[CG]GTGCAGCAGCC[CG]
G[CG]CATTCCCCTCTCTCCCTCCCTCTTGCTCTCCCTCCCTTTC
TGTCTTCCTCTCTTTCCTCCTCTACTGCTCCCTCCCTCTCTTGCC
TCTTAAGTTTCCTGCAC[[CG]]TGAATCCAACTGTGCCAAGCCT
TGGCTCC[[CG]][CG]AACCAATCCTGAG[CG][CG]ACC[CG]
GGCACTGGGA[CG]G[CG]ACTC[CG]CCAAAGCTGGA[CG]AGG
CAGC[CG]GACC[CG]TCTG[CG]CT[CG]AGCATGGAGA[CG]G
AG[CG]CCTGGGAGGGCA[CG]TC[CG]GGG[CG]CTGGAGA[CG
]CCAGGCC[CG]AGTAGCTTCTCCATGGAGCCTGCCCAGAG[CG]
GTCCCTTCT[CG]CAGGATT[CG]CCCCAAGTCCTGTG[CG]GTA
[CG]TACCCTTTGCCTC[CG]CTGTTCC[CG]GAGGGTACACCTG
GGAG[CG]GGGTGGTGGGCA[CG]AGGAAGGGA[CG]GCAGGGAG
GGG[CG]CTGG
SEQ ID NO: 5278
G[CG]CAGGAGCTGCAGC[CG]C[CG]CCTT[CG]CTGGAGCAGC
[CG]AGGGGC[CG]GTGCCACCTTTGCT[CG]CCCCCTCACTACC
ATGTACAGTGTG[CG]CTGCAAGAAGAAGG[CG]GGAGATGCATG
CACACCCC[CG][CG]ACC[CG]CTCCCTCTGGCT[CG]TCCCC[
CG]G[CG]TG[CG]CCACTGACCAGGGGGGAATGTGGTGAAGA[C
G]G[CG]AGGAGGAGAGC[CG]AGGGGGGAGGGGAGGGC[CG]GC
[CG]G[CG][CG]AACCCAGGC[CG]GAGGGTG[CG]AGGAGCCA
GAGGAGGCTGGAGGGGG[CG]G[CG]GCCACAGCTGGGT[[CG]]
GAGAAAGTTGCCCCCTATGCAGATGGCAACTGTGAGTACCCCTGT
GGGGGGGAGGGTGCAG[CG]AAGGCTGCCAGGGACCC[CG][CG]
AT[CG][CG]CCTTAACA[CG]TATTGGAG[CG]AGTGTG[CG]A
GTGATCATGAGTGTGAG[CG]GGTG[CG]AG[CG]CT[CG]G[CG
]GGGCTGAC[CG]TCA[CG]TTGG[CG]GGGC[CG]G[CG]GGGC
CAGGCAGTCTTCTC[CG][CG]TCCCCCTCCTCC[CG]CTC[CG]
TGCCTCCCCTCCCAGCAGCTCCCCCTCCC[CG]CCC[CG]CC[CG
]GCTC[CG]GCTCC[[CG]]GCCCCTAC[CG][CG]AGG[CG]CC
SEQ ID NO: 5279
ATGTCA[[CG]]GTCATGTTGCATCACTTCACTTGTTGAGCTGTG
[CG]AAGGCAGCACTTGACCACAGAAACTTGGTGAAC[CG]CCTT
GCTCTTAAATTAGCCCCCAAGAATTCTCTCCAGCCCTATAGAAAT
GTCTTACCTGCATGTGTCCCTGGTACATTCTGCTT[CG]GTTTCT
TCAGGGCTCCCTGAGGCCAC[CG]GATGCTTTTCCCC[CG]TGCT
TACTTGCCCACCAAAAGAGGGGAAAAGGTGGGTGCACAG[CG]GG
TTCTCAGAGGGTGCTCCTGAGCCCCCTGGAGCTC[CG]GGTACT[
CG]GC[[CG]]GC[CG]G[CG]GCCACT[CG]GCAG[CG]CTG[C
G]GGCTGC[CG]GGAACTGTTCTC[CG]CT[CG]GGGTGCTGAAA
G[CG]GA[CG][CG]GGAGAG[[CG]][[CG]]CAGAGAAGG[CG
]AGGAGC[CG]GGT[CG]GCCAGGCTCTCCTGCAGG[CG][CG]G
GTCCTGCT[CG][CG]GGG[CG]TCTCTAGAACTCACTCCTTCTT
[CG]C[CG]AACTCTTTCT[CG]CTTCCTGCCAGGTTGCCTCATG
TCTCTCTCTCTCTGAGCTG[CG]AACCCCCTCCTCCCAGCCCTGA
[CG]TGAG[CG]CCTACACCAGC[CG]C[CG]C[CG]CAGCTGC[
CG]AG[CG]GGG[CG][CG]C
SEQ ID NO: 5280
[CG]TCTGC[CG]TGTGGTTCTCTTTTCTTC[CG]AAAGGCCAGT
GTCTTATCTCTCCACTTCAAGTCCAGAGGACTTGCTCAGTCTCCT
CCCCTTAAGTCATTTCCACCATCCTCAGGCAGCTGTGGGAAGC[C
G]AGAGTCCTGGACTGTT[CG]TC[CG]GGTGCCAG[CG]CTGGC
AGTCCCAGTC[CG]TC[CG]GTGCAGCAGCC[CG]G[CG]CATTC
CCCTCTCTCCCTCCCTCTTGCTCTCCCTCCCTTTCTGTCTTCCTC
TCTTTCCTCCTCTACTGCTCCCTCCCTCTCTTGCCTCTTAAGTTT
CCTGCAC[[CG]]TGAATCCAACTGTGCCAAGCCTTGGCTCC[[C
G]][[CG]]AACCAATCCTGAG[CG][CG]ACC[CG]GGCACTGG
GA[CG]G[CG]ACTC[CG]CCAAAGCTGGA[CG]AGGCAGC[CG]
GACC[CG]TCTG[CG]CT[CG]AGCATGGAGA[CG]GAG[CG]CC
TGGGAGGGCA[CG]TC[CG]GGG[CG]CTGGAGA[CG]CCAGGCC
[CG]AGTAGCTTCTCCATGGAGCCTGCCCAGAG[CG]GTCCCTTC
T[CG]CAGGATT[CG]CCCCAAGTCCTGTG[CG]GTA[CG]TACC
CTTTGCCTC[CG]CTGTTCC[CG]GAGGGTACACCTGGGAG[CG]
GGGTGGTGGGC
SEQ ID NO: 5281
CAC[CG]GACAGAAATGTCA[[CG]]GTCATGTTGCATCACTTCA
CTTGTTGAGCTGTG[CG]AAGGCAGCACTTGACCACAGAAACTTG
GTGAAC[CG]CCTTGCTCTTAAATTAGCCCCCAAGAATTCTCTCC
AGCCCTATAGAAATGTCTTACCTGCATGTGTCCCTGGTACATTCT
GCTT[CG]GTTTCTTCAGGGCTCCCTGAGGCCAC[CG]GATGCTT
TTCCCC[CG]TGCTTACTTGCCCACCAAAAGAGGGGAAAAGGTGG
GTGCACAG[CG]GGTTCTCAGAGGGTGCTCCTGAGCCCCCTGGAG
CTC[[CG]]GGTACT[CG]GC[[CG]]GC[CG]G[CG]GCCACT[
CG]GCAG[CG]CTG[CG]GGCTGC[CG]GGAACTGTTCTC[CG]C
T[CG]GGGTGCTGAAAG[CG]GA[[CG]][CG]GGAGAG[[CG]]
[[CG]]CAGAGAAGG[CG]AGGAGC[CG]GGT[CG]GCCAGGCTC
TCCTGCAGG[CG][CG]GGTCCTGCT[CG][CG]GGG[CG]TCTC
TAGAACTCACTCCTTCTT[CG]C[CG]AACTCTTTCT[CG]CTTC
CTGCCAGGTTGCCTCATGTCTCTCTCTCTCTGAGCTG[[CG]]AA
CCCCCTCCTCCCAGCCCTGA[CG]TGAG[CG]CCTACACCAGC[C
G]C[CG]C[CG]CAGCTGC
SEQ ID NO: 5282
GAAAGGGAAAGAGAAAGCC[CG]GGC[CG]CCAGAG[CG]CTTAA
TCACA[CG]TACATCCTAC[CG]GGCCCCTCTCCAGCTGGCTCAG
GCTCCACACTGGTCCTCCAGCTCCCTTTTCTCAG[CG]ATGGACT
CACAGCCCCACC[CG]GAG[CG]CTGGAG[CG][CG]GA[CG][C
G]GTCACTG[CG][CG]TG[CG]CCTCAC[CG][CG]CTGGCACC
C[CG]GCCTGGCAGCCTTTGGGGACCTGAACCAGCTG[CG]CCTG
[CG]CAGGTGGAA[CG]GGTGGAA[CG]GGTGGGGGAG[CG]GAC
AGT[CG]AA[CG]GCCTGAGAGGGCTCAGCTGGTC[[CG]]GGTG
GGATTCTGAAGGCCAG[CG]GAGGTGGGCTTACTAGGGAAAGGGG
[CG]CAGCAGTAACTACC[CG]CAACTGAG[CG]G[CG]GGATCT
G[CG]GC[CG]CC[CG]GGGCCCT[CG]GCCAACATCCCAGCATC
CTCTG[CG]C[[CG]][CG]C[CG]GCCC[CG]GACTCCATTTCC
CAG[CG]GCCCCTG[CG]GCCTGCCCTAGGGCTG[CG]G[CG]CC
TTTGTGAG[CG][CG]GC[CG]C[CG]GCCAGGAT[CG]AGCCCT
GGCC[CG]GGCCCTGGCCCAGCCC[CG]GCCTCCAAGGAC[CG][
CG]C[CG]AAGGAGGTGCCCACTGGAGGGAGGAGG
SEQ ID NO: 5283
GCTTAC[CG][CG][CG]CA[CG]CCTGGCTGGAGAGGTGA[CG]
C[CG]CTGTCTGCCAACCTTTCCCAGCTTTTCCCA[CG]ATTGGA
GCCAAGAGAGGTCCC[[CG]]GGGATCTCAGTTGGGCAATTGTAA
GGGAAGGAGCTCAGGACACTGACTTCAAGATGAAGGGGGAGGTCA
G[CG]AAGACAGTTCTAGGGTGGG[CG]G[CG]GC[CG]CTGACA
GAGCAGGAGCCACAGCCAC[CG]GGGCTTGGAGATAGGAGCAAGT
[CG]CAGG[CG]GAGGGGGCTG[CG]GGCTGGCTGCCTGGGCAGC
ACAGGACTTGAGGGAGCTG[[CG]]GGGACTCCTGGAGTCTCATC
AGGCCTTCCAGT[[CG]]CTGTGGGGACCC[CG]GCTG[CG][CG
][CG]GAT[CG]CCTG[CG]CCACTGTCCCCACTGACC[CG]CC[
CG]C[CG]GGTTTGCCAATTACCAG[CG]CCACCTGGTCC[CG]C
ACCCAACCCCACATTTTCTCTGTCAAGGGCTTGTTTCCAGGGCTC
CAGGAACTTTCAAAATGTTATTGGTGTGTGT[CG]GGGGGGTGGG
GGG[CG]GGGGGGTTGTGTGGGGG[CG]GGAGTGTAAAGGAGGAG
GGAACTCTTCTTTTCTCTTTGTCTTTGTAAATGGACACCT
SEQ ID NO: 5284
GAGGCCTGACTTGCAAGCAGCTTTCTT[CG]CCT[CG]TTCT[CG
]GGGCTTCAGGGAGG[CG]G[CG]ACTGGAGCCC[CG]CTGCCTA
CTCACAACAAAAGAAAGCAGGGCTGG[CG]CCCCTACA[CG][CG
]CTCCCATTCACTGC[CG]GC[CG][CG]GGGAGAATCCTACCC[
CG][CG]CCCT[CG]CCAGGGTCC[CG]GGGAGCTCTGGGCTGG[
[CG]]GAGG[CG]AACAAGATG[CG]GTTTGACAGAACCAGGC[C
G]TGCTTCTGCAAACCTCAATGC[CG]CTC[CG]CCAGC[CG][C
G]G[CG]GCCTCCCAGACTCCATTTGC[CG]GGGAGCTC[CG]AG
C[CG][CG]CAAAATCTCTGCAAGCTGTGCTCTGCAGAATGCAAA
GC[CG]GGCTGGAATC[CG]CCATTCC[CG]C[CG]CCCCAGC[C
G]ACT[CG]GC[CG]A[CG]AGCTCCCTCCTGAGCCTCC[CG]GG
CCATCTGC[CG]GGA[CG]GTTACCT[CG]TT[CG]GTGAAGC[C
G]GTGCATTCACCACATCCTCTGCTGCTCCTAGCAGGGAAGC[[C
G]]G[CG]GTGAGG[CG]GGGGAGCAGGCATGGTGGCTC[CG]GG
GTTTATG[CG]GGGTTTACTGCTTACCCAGAATAG[CG]GCTACT
GCTGCCTACTAGGG[[CG]]CACCTAC[CG]CT
SEQ ID NO: 5285
TCTTCATGCCTCAGAAA[CG]TGGCCTACTCTGCATT[CG]GTGT
GTG[CG]GAAGCAGCAATCACAGAGGCAGCCCTAATAC[CG]GAG
G[CG]G[CG]G[CG]GCAGCAGCAGGGCCAGGTGGTAGCT[CG]G
GGCTGAGGAT[CG][CG]G[CG]GGGGCAGC[CG]CTATGGGGCC
CAAGCCCTGACACA[CG]TACCATT[CG]CTCAAGT[CG]G[CG]
GTA[CG][CG]C[CG]CCAC[CG][CG][CG]AGGAGGCCACCTG
GGACTTGTGGC[CG]CAGTC[CG]A[CG]AGGG[CG]ACA[CG]A
GGGCAGA[CG]GTGTAGC[CG]AAT[CG]TAGCCAGAGCTGGG[[
CG]]G[CG]G[CG]AG[CG]CC[CG]TGCACCTTCATGTGCTTA[
CG]CAG[CG]AGCTGGGGTG[CG]TGTAGCACTTGT[CG]CAGCC
C[[CG]]CACCTTGCA[CG]TGTATGGCTTGT[CG]CTAGTGTGC
A[CG]TG[CG]AATGCTTCTTA[CG]GT[CG]CTGCTGTTGG[CG
]AAG[CG]C[CG]CT[CG]CAGCCCT[CG]AACT[CG]CATCTGA
AGGGCTTCT[CG]CCTGG[CG]GAGGCAA[CG]CAGAGACATTAG
TGCTTGTGGGT[CG]TGTTCC[CG]TCAGGTGCTTGCCACCCTCC
CCCATT[CG]TCTCTCATTTTCTGGAAAAGAACTACAAAATAT
SEQ ID NO: 5286
CCCCAGACCCAGAGCTGCCC[CG][CG]GATGAGGAGGGGG[CG]
TGGCTGAGGGACA[CG]GTGTTTGTTCCCCATCCCTGAGTTC[CG
]TG[[CG]]GACCTGCCCCAGCCCCAGCACAGTCACCCTGTGGAA
CTGGCATCCAGGAGAGGTGCTC[CG]CTCTGTG[CG]CTCCTTCC
TCC[CG]GCTGTGTGCTCCCTGTCCACAGCTCCCCATCCACAGCT
T[CG]GCACTGTGGGGCTGGTGCTCCAGGAGGGAAGGCC[CG]GT
TCTTTGCATCCCCTTTC[CG]CAAC[CG]CTAGGGTCTCTGTG[C
G]GGCCCTAGAGGAA[[CG]]CTCACTCAT[CG][CG]CAGCTCA
CCAGGGACCCCAGCTTGGCAGAAACCAGGGGCTCTCTG[CG]GGT
TCTCTCAAATGAGTCCTTTG[CG]GTGGGGGAG[CG][CG]GATG
GGGAGGTGGGTG[CG]TCCCT[CG][CG]A[CG]GGAACTACCTC
ACCAG[CG]GTCCTGTC[CG]GGGCTCCTTCACCTCAGTGTCCTC
TTCTCTACAATGGGAGTGA[CG]GCCCTGCTTTGGGTTGGAGGAA
TTTAAAGGAGTTAA[CG]TATGG[CG]CC[CG]CAGGGCTCA[CG
]AAAGGTAGC[CG]TGTGGTTTTTC[CG]ACCTTTTTTATTA
SEQ ID NO: 5287
ATAC[CG]GAGG[CG]G[CG]G[CG]GCAGCAGCAGGGCCAGGTG
GTAGCT[CG]GGGCTGAGGAT[CG][CG]G[CG]GGGGCAGC[CG
]CTATGGGGCCCAAGCCCTGACACA[CG]TACCATT[CG]CTCAA
GT[CG]G[CG]GTA[CG][CG]C[CG]CCAC[CG]C[CG]C[CG]
AGGAGGCCACCTGGGACTTGTGGC[CG]CAGTC[CG]A[CG]AGG
G[CG]ACA[CG]AGGGCAGA[CG]GTGTAGC[CG]AAT[CG]TAG
CCAGAGCTGGG[CG]G[CG]G[CG]AG[CG]CC[CG]TGCACCTT
CATGTGCTTA[CG]CAG[CG]AGCTGGGGTG[CG]TGTAGCACTT
GT[CG]CAGCCC[[CG]]CACCTTGCA[CG]TGTATGGCTTGT[C
G]CTAGTGTGCA[CG]TG[CG]AATGCTTCTTA[CG]GT[CG]CT
GCTGTTGG[CG]AAG[CG]C[CG]CT[CG]CAGCCCT[CG]AACT
[CG]CATCTGAAGGGCTTCT[CG]CCTGG[CG]GAGGCAA[CG]C
AGAGACATTAGTGCTTGTGGGT[CG]TGTTCC[CG]TCAGGTGCT
TGCCACCCTCCCCCATT[CG]TCTCTCATTTTCTGGAAAAGAACT
ACAAAATATTTTCAGAAATCCCTTTCCA[CG]G[CG]CCTCAGGT
[CG]AGCACCCCTTTCCCT[CG]TGCAGAGAG[CG]CCCC[CG]G
TGCC
SEQ ID NO: 5288
GG[CG]GGGAGAGGAGTCCCTCCCCAACTTCACTCCCCTAATCAA
GC[CG][CG]GGTGAGATAGGCAGC[CG]G[CG]GTTTGTGCCCA
TCTGTGGGGGCAAGTCCTACCTTCTGCCAAAAGG[CG][CG]A[C
G]CATGCACAAAAGATGGATCCAGGCTATCTTTCTCTGCCATCAG
CTCAGGCAAATATTTCTCCTCTTCCATAG[CG][CG]GACTT[CG
]GATTGTCCC[CG]GG[CG]AAG[CG][CG]AGGTTC[CG]CT[C
G]CT[CG]GA[CG]CAGGCAGGGTCTTGGGGCAG[CG]CCTGGCT
CC[CG][CG]CTGCTCCTCCTC[CG][CG][CG]G[[CG]]AGGG
ATCTCTGTG[CG]TCCTCACTGGCCCATGCACCCAGCACCTG[CG
]ACTCC[CG]C[CG]T[CG]GGCTG[CG]TGGCCC[CG][CG]CC
CACACCTGCC[CG]TCCCTTC[CG]T[CG]TCCCT[CG]CT[CG]
[CG]CAGAGCCC[CG]GCTCACACCAG[CG]GCCTTAACTGGAGA
GG[CG]GGAACAGGA[CG][CG]GCCCACCTC[CG]CCAACCACT
CATTGGTGGTGGTGCCAGGAGAGG[CG]GGG[[CG]]CTGGCAGA
AGGC[CG][CG]GGCTCTCATTGGG[[CG]]GTAG[CG]CAAGT[
CG]GGTGGCCTGACTGGCAGGAGGCAGCTGCAGAGGG
SEQ ID NO: 5289
CCCCCTTCCTGTATTAGCATG[CG]GG[CG]G[CG]G[CG]AGC[
CG]GC[CG]GGTGGGGAGGG[CG]G[CG]GTGGCAT[CG]CTG[C
G][CG]GGG[CG]CATTGTGGGC[CG][CG]CT[CG]CCTC[CG]
[CG]GGGGACCATCTGCT[CG]CTGTCAATGCATCACCTGCT[CG
]TCTGGGC[CG]T[CG]C[CG]GGGCAA[CG]GGGGG[CG]GGGG
ATTAAGGAG[CG]TGTG[CG]TCT[CG]GTC[CG]GGC[CG]AGG
[CG]G[CG]AGGTGGGGGTTGGGG[CG]GGGGAGGAGAGCTCCTT
GGCCCCCCACCCCCCTGCCC[CG]AGA[CG]GGT[CG]ACC[CG]
CT[CG]GGGGC[CG]G[[CG]]ACCAC[CG][CG]A[CG]GGTTC
[CG]C[CG]CTTGCCTC[CG]CTCCTTGGCCTTTGCTGC[CG]TG
CTGCCTCTTCTCA[CG]GG[CG][CG]GCTGGAGTCC[CG]GGGA
GCAGCAGAGAGCAAA[CG]GTC[CG]GCTCTACCTCACCCTGCCA
GGGGG[CG]AGTCC[CG][CG]CTCCCTG[CG]TCTACCTGGAGC
TGCAGGGTCCCTATCC[CG]GGG[CG]C[CG]CC[CG]CAGCCTC
CTC[CG][CG]GGAGCTGGAGCACTCTGCTGGGTCCTGGGGA[CG
][CG]G[CG]CAG[[CG]]TGGTTCCAAG[CG]CTC[[CG]]CAG
T[CG]C[CG]GGCAACCACAATGGGG
SEQ ID NO: 5290
[CG]AG[CG][CG]AG[CG]CAAC[CG][CG]TCAAGTTGGTCAA
CCTGGGCTTTGCCACCCTT[CG]GGAGCA[CG]TCCCCAA[CG]G
[CG][CG]GCCAACAAGAAGATGAGTAAGGTGGAGACACTG[CG]
CT[CG]G[CG]GT[CG]AGTACATC[CG][CG][CG]CTGCAGCA
GCTGCTGGA[CG]AGCATGA[CG][CG]GTGAG[CG]C[CG]CCT
TCCAGGCAGG[CG]TCCTGT[CG]CCCACCATCTCCCCCAACTAC
TCCAA[CG]ACTTGAACTCCATGGC[CG]GCT[CG]C[CG]GTCT
CATCCTACT[CG]T[CG]GA[CG]AGGGCTCTTA[CG]ACC[CG]
CTCAGCCC[CG]AGGAGCAGGAGCTTCT[CG]ACTTCACCAACTG
GTTCTGAGGGGCT[CG]GCCTGGTCAGGCCCTGGTG[CG]AATGG
ACTTTGGAAGCAGGTAGGTTGCATTTTGGGGTGGGCAGGGGGGTA
TTCTTGCCTT[CG]TCCTCCCTCTGAGTGTCTGTGGAAGTGGGGA
TGTCTCCAAGGAGATAAGGGGATTTTTATTTAAAGAATTTGTGAA
AGTTGGT[CG]ATTTCAAGTCCTAGTTTGTTAGTTTCAGCACTGG
CCTCTGAAAATGGCCTTGCCCAGGTCTCCAAGGAGTGAAGGGTAG
TAGTG
SEQ ID NO: 5291
C[CG]GAGAGACCT[CG]GAGCAGAGAAGG[CG]C[CG]C[CG]A
CCCT[CG][CG]GCTGCCTGGCC[CG][CG]GCTCCTACAAAGG[
CG]GGCTAGC[CG]CC[CG]CCCTCTCCCTTGCCTTCCTCCCCTT
CTTTTCTGACTTTCCCTCTTTCCCTTAAT[CG]CCTGCTTCTTCC
TC[CG]GGTGGACTTA[CG]GCCACCTTGCTCCTC[CG][CG]CT
TCACCTCAT[CG]CCCCCTCTTTCTTTCTTCTGCCTCTCTCTCTG
[CG]CCCCCTTCTCTC[CG]TGTCA[CG]CTCCCTCCTGGTTCTG
[[CG]][CG]TCTACAAACTTTTGAGCAGAACA[[CG]]AGCCT[
CG]GCAAA[CG]AGTCC[CG]CAGCTCCTCCTGCTGCTCC[[CG]
]CTGGTTCCTG[CG]GCTTCTGCTCAGACACCAA[CG]CCAGA[C
G]G[CG]ATGCCTCT[CG]GGTGGTGACTCCAG[CG]CAGGAACT
TGAAGAAG[CG]CTTTGCC[CG]C[CG]TCCTACCTGGCAGCTCT
CCTGGCAG[CG]GGAGGAGTTGAAGGGTAAGGGAGGGAAAATCTT
ACCAAAG[CG]AC[CG]GCTCACT[CG]ACTGCTGATTCTTT[CG
]CTTGGCAT[CG][CG]TCAGGGGAGTTAGCTTTCCTTCAGC[[C
G]]GGTCTGGCTAGTTATTGGG[CG]
SEQ ID NO: 5292
TTGCTCTTAAATTAGCCCCCAAGAATTCTCTCCAGCCCTATAGAA
ATGTCTTACCTGCATGTGTCCCTGGTACATTCTGCTT[CG]GTTT
CTTCAGGGCTCCCTGAGGCCAC[CG]GATGCTTTTCCCC[CG]TG
CTTACTTGCCCACCAAAAGAGGGGAAAAGGTGGGTGCACAG[CG]
GGTTCTCAGAGGGTGCTCCTGAGCCCCCTGGAGCTC[CG]GGTAC
T[CG]GC[CG]GC[CG]G[CG]GCCACT[CG]GCAG[CG]CTG[C
G]GGCTGC[CG]GGAACTGTTCTC[CG]CT[CG]GGGTGCTGAAA
G[CG]GA[[CG]][CG]GGAGAG[CG][CG]CAGAGAAGG[CG]A
GGAGC[CG]GGT[CG]GCCAGGCTCTCCTGCAGG[CG][CG]GGT
CCTGCT[CG][CG]GGG[CG]TCTCTAGAACTCACTCCTTCTT[C
G]C[CG]AACTCTTTCT[CG]CTTCCTGCCAGGTTGCCTCATGTC
TCTCTCTCTCTGAGCTG[[CG]]AACCCCCTCCTCCCAGCCCTGA
[CG]TGAG[CG]CCTACACCAGC[CG]C[CG]C[CG]CAGCTGC[
CG]AG[CG]GGG[CG][CG][CG]C[CG]TTCTCCCAGC[CG][C
G]TGCCTGC[CG]AGAGGC[CG][CG]GGAC[CG]GGCA[CG][C
G][CG][CG][CG]TTCCCAGGGGCCTGGGAG[CG]GCC
SEQ ID NO: 5293
CTGGCCTTGGTAGA[CG][CG]GGCTTCACTACTAG[[CG]]TGG
TGC[CG]C[CG]CTGCTGGCCAACCTG[CG][CG]GACCAG[CG]
CTCTGGCTGC[CG][CG]CAGCCACTGCA[CG]GCCCAGCTGTG[
CG]CAT[CG]CTGGCTCTGGGTTC[CG]C[CG]AATG[CG]TCCT
CCTGG[CG]GTGATGGCTCTGGAC[[CG]][CG][CG]GC[CG]C
AGTGTGC[CG]CC[CG]CTG[CG]CTATG[CG]GGGCT[CG]TCT
CCC[CG][CG]CCTATGT[CG]CA[CG]CTGGCCAG[[CG]]CCT
CCTGGCTAAG[CG]GCCTCACCAACT[CG]GTTG[CG]CAAAC[C
G][CG]CTCCTGGCTGAG[[CG]]GC[CG]CTGTG[[CG]][[CG
]]CCC[CG]CCTGCTGGACCACTTCATCTGTGAGCTGC[CG]G[C
G]TTGCTCAAGCTGGCCTG[CG]GAGG[CG]A[CG]GAGACACTA
C[CG]AGAACCAGATGTT[CG]C[CG]CC[CG][[CG]]TGGTCA
TCCTGCTGCTGC[CG]TTTGC[[CG]]TCATCCTGGCCTCCTA[C
G]GTGC[CG]TGGCC[CG]AGCTGTCTGTTGCATG[CG]GTTCAG
[CG]GAGGC[[CG]]GAGGAGGG[CG]GTGGGCA[CG]TGTGGGT
CCCACCTGACAGC[CG]TCTGCCTGTTCTA[CG]GCT[CG]GCCA
TCTACACCTACCTGCAGCC[CG][CG]CAG
SEQ ID NO: 5294
AGGGG[CG]AGGCT[CG]AGGGTCCCTAGGGGTGGTGGGA[CG]C
AAGGCCCATGCC[CG]TCTATGGCCTCT[CG]C[CG]C[CG]GCA
GCT[CG]CAGCCACC[CG]AGCAT[CG]CCT[CG]G[CG][CG]G
GC[CG][CG]A[CG]CTCTC[CG]CCC[CG]AGGGCA[CG]CTCC
[CG]GGGCTCTTGGC[[CG]]CCCCT[CG]CCCAC[CG]GGCTCT
GGGTAGCCCCTCACCAGGCTCTTGG[CG]GCCACCTAGCC[CG]G
[CG]CC[CG]GCCCCCTG[CG]GC[CG]GCCCATTTAGTGTGAAT
CCTGGATC[CG]GCAG[CG]G[CG]G[CG]GCTTCTTCAGGGGTA
GCTGATGC[CG]GGGCCTTGGAGAGGGA[CG]CTTTGGGAGGGTC
CTGGGAGG[CG]G[CG][CG]G[CG]AGCAAAGGGGGCAAGAAGG
G[CG]TGCTGTGCTCC[CG]CCTGGCTCC[CG]GGG[CG]T[CG]
TTTGGGGG[CG]GC[CG]GGGAGTTTTCTACTTGGAAGCCCCTCT
GAAGCCAC[CG][CG]TCTCCTTTGCC[CG]GCCT[CG]TCTC[C
G]CTGCCCA[CG]CAGGCTGC[CG][CG]CTCTGGCCT[CG]GCC
C[CG]GCTCT[CG]GAGCTGCC[CG]G[CG]GGCTC[CG]GGTCT
[CG]CCCTCCAGCCCCCAGG[CG]GCC[CG][CG]G[CG]GATCC
C[CG]AGAGGCAGC
SEQ ID NO: 5295
TGCTCTTTCCCCCTC[CG]GAAGCCCT[CG][CG]TCCTCATCCT
TACCCCACCTCTTGTTCCCCAAG[CG]TGGCCAGGGCTAAGGCTC
CAGGGATG[CG]CCAAGCACCCTT[CG]GTTTTCC[CG]GGGAGA
ATTTTCCC[CG]GCC[CG]GGGACTAGGGTCTGG[CG]CTGGGG[
CG]CCCCT[CG]GACCTG[CG]GGAT[CG]CCCCTACACTCTGG[
CG][CG]CTGAGGG[CG]GTGAG[CG]AGGG[CG]CCAAGGCACA
GGTGGGG[CG]GGAGT[[CG]]AG[[CG]][[CG]]GAGGCT[CG
]GGGGG[CG]GGA[CG][CG]GGGCCTGGGAG[CG]GCCAGGGAC
[CG][[CG]]GCAG[CG]CCTCAGTGCCAGCCTGG[CG]CC[CG]
[CG]ACTGCCTGCCCCAGCCCCTCAGTGG[CG]GCTTGCTCTCTT
CTCT[CG]CTC[CG]AACCAGACACAGC[CG]CTGC[CG]CTGC[
CG]TC[CG]G[CG][CG]CTACAGACTCC[CG]AGAACAGCCCTG
GCTGTCAG[[CG]]AGCACCAGC[CG]CTTCCTGTCCCCAT[CG]
[CG]GAGACTGGAGGGG[CG]CACCA[CG]GCCATGGAGCCAGAG
G[CG]CTTCAGGAGGCAAGAGAAGTCCC[CG][CG][CG]CTC[C
G]CAGCC[CG]G[CG]CAGCTCATGGTGAG[CG]CCCTCTGGGGC
T[CG]AGG
SEQ ID NO: 5296
TCTT[CG]TTTTCTTAGATTTCAAACTTGCAAGGAT[CG]CAAGG
ATCCAGGGCCCCAGGAAAGGAGGGGTGTGAAGGACTCAAAATTCC
AGCAGCTTGGCTGGGGTGGCTG[CG]CCAGA[CG]GGCCCTTCCC
CAAAGTGCAAACCCACCCCTGTCCT[CG]GCCC[CG]G[CG][CG
]CTCCCTCCCTCAGCC[CG]GGGC[CG]TACACCACCTGCCCTCT
AC[CG]AGAGATCTGGG[CG]G[CG]G[CG]GC[CG]AAAGCAG[
CG]A[CG][CG]CC[CG]GAGCATCCCTTG[CG]ATA[CG]CTAG
GGGA[CG]GTGCTTTCTCTGTCCCAGTTG[[CG]]TG[CG]G[CG
]GGGCTGGGGCCCAGGC[[CG]]CCCCAAATCTCCCCCACTAGAG
TGACCAC[CG]CACAGTTGTCCC[CG]CTGGG[CG][CG]CTCCT
C[CG]GTGTCTG[CG]CTCAGC[CG]CTCTCCTCTTCTCTCTCC[
CG]CC[CG]CC[CG]CAG[CG]CCATGGTCTGGCAGTGTGTTTAG
[[CG]]CTCACCACCATGTGCACCAGCTTGTTGCTAGTGTACAGC
AGCCT[CG]G[[CG]]GCCAGAAGGAG[CG]GCCCC[CG]CAGCA
GCAGCAGCAGCAGCAGCAACAGCAGCAGCAGG[CG]T[CG]GCCA
C[CG]GCAGCT[CG]CAGC[CG]G[CG]G
SEQ ID NO: 5297
GCCCCTTTCCCTAA[CG]CTGCCCACTGAGG[CG]TCCTCAC[CG
]TCTACCCTGTTGGGGT[CG]CTCTCTGACCCC[CG][CG]CTGC
ATCCC[CG]GGAC[CG]ACCCACCTGCTG[CG]CAGCTACCCCAG
GC[CG]GTCCCAGC[CG]CC[CG]GAGCCCCAGTG[CG][CG]AT
GG[CG]GC[CG]GCAAACTG[CG]CCTG[[CG]]CACTGGGCCTC
AC[CG][CG]GACTA[CG]ACTCCCACAATGC[CG][CG]AGGCT
GTGC[CG][CG]CAC[CG]GGG[CG]AGCAAGGGAGC[CG]TGAC
AGGTGACAGTG[CG]CATGGCCAATCAGA[CG][CG]AGTGTGGC
CTCC[[CG]]G[CG]TCC[CG]CCCACTC[CG]G[[CG]]CCTG[
CG]TCATTTTGGGCACCATGGCAA[[CG]]AAGTAAACAAG[CG]
GGTAAATAATAGACCCCTGACAGCCAGAGACC[CG]GAGAAACAA
AGA[CG]GTGAGAAA[CG]GAGAAACAAAGACAAAAAGA[CG][C
G]CAGAC[CG]GGAGGGAGAGAAGCATGAAGGCCCACAGA[CG]C
T[CG]GGGGCAGACCAAGA[CG]CCCTGGGGTCCC[[CG]]AAAC
AGAGATGTACAGAGT[[CG]]GACA[[CG]]GAAAGGGAGAGACC
AGCTCA[CG]GTTAAGAAAGGAGGGAGACCAGCCCAGGATCAA
SEQ ID NO: 5298
GTGAAC[CG]CCTTGCTCTTAAATTAGCCCCCAAGAATTCTCTCC
AGCCCTATAGAAATGTCTTACCTGCATGTGTCCCTGGTACATTCT
GCTT[CG]GTTTCTTCAGGGCTCCCTGAGGCCAC[CG]GATGCTT
TTCCCC[CG]TGCTTACTTGCCCACCAAAAGAGGGGAAAAGGTGG
GTGCACAG[CG]GGTTCTCAGAGGGTGCTCCTGAGCCCCCTGGAG
CTC[[CG]]GGTACT[CG]GC[[CG]]GC[CG]G[CG]GCCACT[
CG]GCAG[CG]CTG[CG]GGCTGC[CG]GGAACTGTTCTC[CG]C
T[CG]GGGTGCTGAAAG[CG]GA[[CG]][CG]GGAGAG[[CG]]
[[CG]]CAGAGAAGG[CG]AGGAGC[CG]GGT[CG]GCCAGGCTC
TCCTGCAGG[CG][CG]GGTCCTGCT[CG][CG]GGG[CG]TCTC
TAGAACTCACTCCTTCTT[CG]C[CG]AACTCTTTCT[CG]CTTC
CTGCCAGGTTGCCTCATGTCTCTCTCTCTCTGAGCTG[CG]AACC
CCCTCCTCCCAGCCCTGA[CG]TGAG[CG]CCTACACCAGC[CG]
C[CG]C[CG]CAGCTGC[CG]AG[CG]GGG[CG][CG][CG]C[C
G]TTCTCCCAGC[CG][CG]TGCCTGC[CG]AGAGGC[CG][CG]
GGAC[CG]GGCA[CG][CG][CG][CG][CG]TTCCCAGGGGCCT
SEQ ID NO: 5299
GAG[CG]CA[CG]GG[CG][CG]G[CG]GGGAGGGTACTCACTGT
GAAGCA[CG]CTG[CG]CCCATGGATCATGTCTGTG[[CG]]TTA
CACCAGAGGCTC[CG]GGCTCCACTAATTCCATTTAGAGA[CG]G
GAAGACTTCCAGTGG[CG]GGGGGAGGACAGGGT[CG]AGAGGTG
TTAAAGA[CG]CAAAGCAAGAAGGAAATAAAGGGGGGC[CG]AGA
GGGAGAC[CG]AGAGGAAGGGGGAGCTC[CG]AGCCCA[CG]CTG
CAGCCAGATC[CG]GATGAGTC[CG]TCCTC[CG]CCC[CG]GG[
CG]GGCTCT[CG]CTCT[CG]CTGGCCCTCAG[CG]C[CG][CG]
CAGCCAGCAGCATCCCCAC[CG]TGA[CG]CT[CG]CATCACACC
[CG]GG[CG]C[CG]GC[CG]CCACCATC[CG][CG]C[CG]C[C
G]C[CG]TCAGGACCCTCCTCC[CG]GGCAT[CG]T[CG]C[CG]
C[CG][CG]GGGT[CG]GGAGGA[CG][CG]G[CG][CG][CG]G
GAGG[CG]G[CG]GT[CG]CAGGG[CG]AGCCC[CG]GGA[CG]C
CC[CG]AGC[CG]GGGC[CG]GGGC[CG]GGGAGAGGG[CG]CAG
[CG]AGGTGGGGGCCAGTCCAGAC[CG]A[CG]GCAG[CG]A[[C
G]]GAG[CG]GG[CG]G[CG]G[CG]G[CG]G[CG]C[CG]G[CG
]G[CG]G[CG]GGGTGGCTCAGTCCCCAGTCTCAGA
SEQ ID NO: 5300
GCTCTTAAATTAGCCCCCAAGAATTCTCTCCAGCCCTATAGAAAT
GTCTTACCTGCATGTGTCCCTGGTACATTCTGCTT[CG]GTTTCT
TCAGGGCTCCCTGAGGCCAC[CG]GATGCTTTTCCCC[CG]TGCT
TACTTGCCCACCAAAAGAGGGGAAAAGGTGGGTGCACAG[CG]GG
TTCTCAGAGGGTGCTCCTGAGCCCCCTGGAGCTC[CG]GGTACT[
CG]GC[[CG]]GC[CG]G[CG]GCCACT[CG]GCAG[CG]CTG[C
G]GGCTGC[CG]GGAACTGTTCTC[CG]CT[CG]GGGTGCTGAAA
G[CG]GA[[CG]][CG]GGAGAG[CG][[CG]]CAGAGAAGG[CG
]AGGAGC[CG]GGT[CG]GCCAGGCTCTCCTGCAGG[CG][CG]G
GTCCTGCT[CG][CG]GGG[CG]TCTCTAGAACTCACTCCTTCTT
[CG]C[CG]AACTCTTTCT[CG]CTTCCTGCCAGGTTGCCTCATG
TCTCTCTCTCTCTGAGCTG[CG]AACCCCCTCCTCCCAGCCCTGA
[CG]TGAG[CG]CCTACACCAGC[CG]C[CG]C[CG]CAGCTGC[
CG]AG[CG]GGG[CG][CG][CG]C[CG]TTCTCCCAGC[CG][C
G]TGCCTGC[CG]AGAGGC[CG][CG]GGAC[CG]GGCA[CG][C
G][CG][CG][CG]TTCCCAGGGGCCTGGGAG[CG]GCC
SEQ ID NO: 5301
AGG[[CG]]TCCTCAC[CG]TCTACCCTGTTGGGGT[CG]CTCTC
TGACCCC[CG][CG]CTGCATCCC[CG]GGAC[CG]ACCCACCTG
CTG[CG]CAGCTACCCCAGGC[CG]GTCCCAGC[CG]CC[CG]GA
GCCCCAGTG[CG][CG]ATGG[CG]GC[[CG]]GCAAACTG[CG]
CCTG[CG]CACTGGGCCTCAC[CG][CG]GACTA[CG]ACTCCCA
CAATGC[CG][CG]AGGCTGTGC[CG][CG]CAC[CG]GGG[CG]
AGCAAGGGAGC[CG]TGACAGGTGACAGTG[CG]CATGGCCAATC
AGA[CG][CG]AGTGTGGCCTCC[CG]G[CG]TCC[CG]CCCACT
C[CG]G[[CG]]CCTG[CG]TCATTTTGGGCACCATGGCAA[CG]
AAGTAAACAAG[[CG]]GGTAAATAATAGACCCCTGACAGCCAGA
GACC[CG]GAGAAACAAAGA[CG]GTGAGAAA[CG]GAGAAACAA
AGACAAAAAGA[CG][CG]CAGAC[CG]GGAGGGAGAGAAGCATG
AAGGCCCACAGA[CG]CT[CG]GGGGCAGACCAAGA[CG]CCCTG
GGGTCCC[[CG]]AAACAGAGATGTACAGAGT[[CG]]GACA[[C
G]]GAAAGGGAGAGACCAGCTCA[CG]GTTAAGAAAGGAGGGAGA
CCAGCCCAGGATCAAGAAAGGGAAAGACCAGCATGGGCTCA
SEQ ID NO: 5302
TGGGGCCCTC[CG]CTG[CG]CACT[CG]GCCCTG[[CG]]C[CG
]AGCACCCTGCAGCCTCCTCC[CG][CG]ACA[CG]G[CG]CTTT
GAACT[CG]G[CG]GATTGATTTTGCTTCCCTTCCCCCTTTTGTG
TGTGTTTG[CG]TTCAATTGGTTAGGTTTTTAAGATTTGGGAGGG
CTGGTGTGAAAGAATTAAAATACTCTTAACTGGAGCCCCTC[CG]
C[CG]AGAACTGGAGGTCC[CG]CCTCCTAGTT[CG]G[CG]CTT
TCAGGACCCTCTTCCCAGAGGGAATTTCTTTCAGAAATTCCAGGG
GGGCTTGTAAAAGA[CG]CTTC[CG]CAGAGCAGGTCC[CG]TCA
GGGTCTTTTTCCTGTTCCTGGTGCCAG[CG]GT[CG]GCC[CG]G
G[CG]CCC[CG]CAGACCT[CG]G[CG]AGGTAGATGTTAAGCT[
CG]GAGAGTGCCCCTCC[[CG]]CAGG[CG]C[CG]TGG[CG]AG
ATCACTCTGAATATGTAACATATTTGTAA[CG]TG[CG]C[CG]A
GGTGTGATGTGTGTGCTGAAATAGGGGGATGGGGGAATT[CG]AA
GC[CG]GATTGGGAAGG[CG]GGGGGGAGG[CG]CACAGAACTCA
CAATGTACTT[CG]CAATCTAACAATCTGAACATTCATTTATTAA
AAGCTGCT
SEQ ID NO: 5303
CCACTGAGG[CG]TCCTCAC[CG]TCTACCCTGTTGGGGT[CG]C
TCTCTGACCCC[CG][CG]CTGCATCCC[CG]GGAC[CG]ACCCA
CCTGCTG[CG]CAGCTACCCCAGGC[CG]GTCCCAGC[CG]CC[C
G]GAGCCCCAGTG[CG][CG]ATGG[CG]GC[CG]GCAAACTG[C
G]CCTG[CG]CACTGGGCCTCAC[CG][CG]GACTA[CG]ACTCC
CACAATGC[CG][CG]AGGCTGTGC[CG][CG]CAC[CG]GGG[C
G]AGCAAGGGAGC[CG]TGACAGGTGACAGTG[CG]CATGGCCAA
TCAGA[CG][CG]AGTGTGGCCTCC[[CG]]G[CG]TCC[CG]CC
CACTC[CG]G[CG]CCTG[CG]TCATTTTGGGCACCATGGCAA[[
CG]]AAGTAAACAAG[[CG]]GGTAAATAATAGACCCCTGACAGC
CAGAGACC[CG]GAGAAACAAAGA[CG]GTGAGAAA[CG]GAGAA
ACAAAGACAAAAAGA[CG][CG]CAGAC[CG]GGAGGGAGAGAAG
CATGAAGGCCCACAGA[CG]CT[CG]GGGGCAGACCAAGA[CG]C
CCTGGGGTCCC[[CG]]AAACAGAGATGTACAGAGT[[CG]]GAC
A[[CG]]GAAAGGGAGAGACCAGCTCA[CG]GTTAAGAAAGGAGG
GAGACCAGCCCAGGATCAAGAAAGGGAAAGACCAGCATG
SEQ ID NO: 5304
AGGCAGATGGGCAGCAGCAGCCTGAAGATGAGCC[CG]CACACCA
CTTCTGAGGCCAT[CG][CG]T[CG]GTC[CG]G[CG]AGT[CG]
GAGCAGAGGGG[CG]AGGCT[CG]AGGGTCCCTAGGGGTGGTGGG
A[CG]CAAGGCCCATGCC[CG]TCTATGGCCTCT[CG]C[CG]C[
CG]GCAGCT[CG]CAGCCACC[CG]AGCAT[CG]CCT[CG]G[CG
][CG]GGC[CG][CG]A[CG]CTCTC[CG]CCC[CG]AGGGCA[C
G]CTCC[CG]GGGCTCTTGGC[[CG]]CCCCT[CG]CCCAC[CG]
GGCTCTGGGTAGCCCCTCACCAGGCTCTTGG[CG]GCCACCTAGC
C[CG]G[CG]CC[CG]GCCCCCTG[CG]GC[CG]GCCCATTTAGT
GTGAATCCTGGATC[CG]GCAG[CG]G[CG]G[CG]GCTTCTTCA
GGGGTAGCTGATGC[CG]GGGCCTTGGAGAGGGA[CG]CTTTGGG
AGGGTCCTGGGAGG[CG]G[CG][CG]G[CG]AGCAAAGGGGGCA
AGAAGGG[CG]TGCTGTGCTCC[CG]CCTGGCTCC[CG]GGG[CG
]T[CG]TTTGGGGG[CG]GC[CG]GGGAGTTTTCTACTTGGAAGC
CCCTCTGAAGCCAC[CG][CG]TCTCCTTTGCC[CG]GCCT[CG]
TCTC[CG]CTGCCCA[CG]CAGGCTGC[CG][CG]CTCTGGCCT[
CG]GCC
SEQ ID NO: 5305
CCACACAGGGTG[CG]GG[CG]GGG[CG]GG[CG]GGGCTGGGGT
GT[CG]GGGGCT[CG]GATT[CG]GAGCCAGCTCTGC[CG]TGGG
G[CG]GGCTGGGGCAGGCTTCC[CG]CTCTGGGAAATTTGCCCCT
GCTCTCCCTGCCAGTTG[CG]GT[CG]TGGG[CG]GCCT[CG]CC
TTGGAGCCCTGGCCA[CG]GAGC[CG]CA[CG]GGG[CG]GTAGA
GAGGCTGGTGGGGAGGGACCAG[CG]GG[CG]GTGGAGGGG[CG]
GCCCTGCAGG[CG]A[CG][CG]GGGACTGGAAAGGG[CG]CCTG
GGTGGGAAGAGG[CG]CTGG[CG]GGTGAT[CG]TCCCCAC[[CG
]]GGCCAGTCCC[CG]GGATCTGCTGC[CG]CCCCTCTC[CG]AA
ATTCACAGCCAGAG[CG]GG[CG]CACTGGTGTGTGTGG[CG]CC
TGGAG[CG]CTAGGGTCCCCACCCAAGTG[CG]AGGCCCC[CG][
CG]GGGAGCCCAGCCCAGGGG[CG]TCCACAGTCCAGGCCCCAGA
CCCCAGGTCCTGGCTAGATT[CG]CCAAAGTCAGAGGCTGGAATC
TGGAGGG[CG]GATGTGAGGGTGCCAGCTAAGCCCAGCCCCTGCC
[CG]G[CG]GTGGCCAGCATAA[CG][CG]GTGACACTCTCTGGA
GCCCCCACTGCCACCACTC
SEQ ID NO: 5306
CAGCTCAATGTGAG[CG]AGCTGA[CG]C[CG]TCCAGCCATGCC
AGTG[CG]CTC[CG]GCAGCAGTA[CG][CG]CAGCAGTC[CG][
CG]CAGCAGT[CG]G[CG]TC[CG]CCTCCCAGTACCACCAGTGC
CACAGCCTGCAGCC[CG]C[CG]CCAGCCCCA[CG]GGCAGCCT[
CG]GCAGTCTGGGCTC[CG]GGCCCC[CG]CTCT[CG]CACCACC
ACCACCACC[CG]CACC[CG]G[CG]CACCACCAGCACCACCAGC
CCCAGG[CG][CG]C[CG][[CG]]AGAGCAACCCCTTCAC[CG]
AAATAGCCATGAGCAGCTGCAGGTACAA[CG]GGGG[CG]TCATG
[[CG]]GC[CG]CTCAGCAACTTGAG[CG][CG]TCC[CG]C[CG
]GAACCTGCA[CG]AGATGGACTCAGAGG[CG]CAGCCCCTGCAG
CCCCC[CG][CG]TCTGT[CG]GAGGAGGTGG[CG]G[CG][CG]
TCCTCCC[CG]TCTGCAGC[CG]CTGC[CG]C[CG]C[CG]C[CG
]CTGTTT[CG]TCCTCAGCCCC[[CG]]AGAT[CG]TGGTGTCTA
AGCC[CG]AGCACAACAACTCCAACAACCTGG[CG]CTCTATGGA
AC[CG]G[CG]G[CG]GAGGCAGCACTGGAGGAGG[CG]G[CG]G
[CG]GTGG[CG]GGAG[[CG]]GGCA[[CG]]GCAGCAGCAGTGG
CACCAAGTCCAGCAAA
SEQ ID NO: 5307
GGCTTCACTACTAG[[CG]]TGGTGC[CG]C[CG]CTGCTGGCCA
ACCTG[CG][CG]GACCAG[CG]CTCTGGCTGC[CG][[CG]]CA
GCCACTGCA[CG]GCCCAGCTGTG[CG]CAT[CG]CTGGCTCTGG
GTTC[CG]C[CG]AATG[CG]TCCTCCTGG[CG]GTGATGGCTCT
GGAC[CG][CG][CG]GC[CG]CAGTGTGC[CG]CC[CG]CTG[C
G]CTATG[CG]GGGCT[CG]TCTCCC[CG][CG]CCTATGT[CG]
CA[CG]CTGGCCAG[CG]CCTCCTGGCTAAG[CG]GCCTCACCAA
CT[CG]GTTG[CG]CAAAC[CG][CG]CTCCTGGCTGAG[[CG]]
GC[CG]CTGTG[CG][CG]CCC[CG]CCTGCTGGACCACTTCATC
TGTGAGCTGC[CG]G[CG]TTGCTCAAGCTGGCCTG[CG]GAGG[
CG]A[[CG]]GAGACACTAC[CG]AGAACCAGATGTT[CG]C[CG
]CC[[CG]][[CG]]TGGTCATCCTGCTGCTGC[CG]TTTGC[CG
]TCATCCTGGCCTCCTA[CG]GTGC[CG]TGGCC[CG]AGCTGTC
TGTTGCATG[CG]GTTCAG[CG]GAGGC[[CG]]GAGGAGGG[CG
]GTGGGCA[CG]TGTGGGTCCCACCTGACAGC[CG]TCTGCCTGT
TCTA[[CG]]GCT[[CG]]GCCATCTACACCTACCTGCAGCC[CG
][CG]CAG[[CG]]CTACAACCAGGCA[[CG]]G
SEQ ID NO: 5308
AGGAGGGGGAGGGGAGGCCAGGC[CG]GGC[CG]GGC[CG]GGC[
CG]GGTG[CG]GGGGGTC[CG]GGGATCTTCCTGAGGCCCTGG[C
G]GGG[CG]AGTTTCCAGCAG[CG][CG][CG]TCTGTGTGGAGT
C[CG]TTTTGCTGCC[CG]GGGCCTGGGGAAGGC[CG]TTT[CG]
GGGCTGG[CG]GGGGCAGGCTTTG[CG]GGGCATCCCTAGTCTGA
GAGGAGGG[CG]GGGCCCAA[CG]CCCAGCTGGACAGGAGCTGCT
C[CG]A[CG]GCCCCTGAGGGAA[CG][CG][CG]CCC[CG]CCC
CTGGCCCCACCTCTGCCCCACAC[CG]GGCACTGGGC[CG]CCAC
CTTTGTTGATGGAAGAGAGAAACTGAGGCATAGTGCCCAGGGCCA
GGGGAC[[CG]]CAACCACC[CG]GCCCTTGTCACTGTAG[CG]T
TAAGGCTCAGAGTCTG[CG]CAGCAGATATGTGTC[CG]CACC[C
G]CCAGGCCTTCAGCTTCCCTCAGACAGG[CG]GGAAAACCCTAG
GCTGCCCTTCTC[CG]AG[CG]GAGGGC[CG]CCCCACACACAGC
AGG[CG]CTTAAA[CG]GGTA[CG][CG]GGGCCCTGGA[CG]GC
TCTC[CG][CG]GAGCTCCCCAGGCTCTGG[CG]CAGTTCCC[CG
]GCTTGGGGACCTGAGCAC
SEQ ID NO: 5309
ATGCAGTTCATTTAGTTAACAAGTGAAATAATGAGGAAG[[CG]]
TGCAGAGTGAATGCCAAGAGAAAAGGCACAAAAACCCTATTGAGA
GGCCC[CG]GC[CG]CTCCTGG[CG]TAGTCCATCACATGGCAAT
AGTCCTATTTAAGC[[CG]][[CG]]C[CG]C[CG]G[CG]CCTT
TCAGCACCACTAG[CG]CTGGCCAGCACCC[CG][CG]CTCTTTG
GG[CG]GTGCCCA[CG]GCAGCAGAGGCTACTGTTTCAGCCTAGG
TCTCAGC[CG][CG][CG]TTCAGCCTCCTGGGCAGAGGCAGCTG
[CG]GGATAC[CG][CG]GCCAGGGAAAG[CG][[CG]]TGGAGA
GC[CG]AAAGGTG[CG]GTGGG[CG]CAGAGGG[CG]GGCTGGCT
G[CG]GGG[CG]AC[CG][CG][CG]C[CG]GGGCCATGC[CG][
CG]CTCCTTCCTGGTGGACT[CG]CTAGTGCTG[CG][CG]AGG[
CG]GG[CG]AGAAGAAGG[CG]CC[CG]AGGGCAGCC[CG]C[CG
]C[CG]CTCTTCCCCTA[CG]CTGTGCCCC[CG]C[CG]CA[CG]
[CG]CTGCA[CG]GTCTCT[CG]CCTGG[CG]CCTGCCA[CG][C
G][CG]CAAGGCTGGGCTGCTGTG[CG]TGTGCC[CG]CTCTG[C
G]TCAC[CG]CCT[CG]CAGCTGCATGGGCCCCC[CG]GGC[CG]
CC[CG][CG]CTGCCT
SEQ ID NO: 5310
GATCTTCCTGAGGCCCTGG[CG]GGG[CG]AGTTTCCAGCAG[[C
G]][CG][CG]TCTGTGTGGAGTC[CG]TTTTGCTGCC[CG]GGG
CCTGGGGAAGGC[CG]TTT[CG]GGGCTGG[CG]GGGGCAGGCTT
TG[CG]GGGCATCCCTAGTCTGAGAGGAGGG[CG]GGGCCCAA[C
G]CCCAGCTGGACAGGAGCTGCTC[CG]A[CG]GCCCCTGAGGGA
A[CG][CG][CG]CCC[CG]CCCCTGGCCCCACCTCTGCCCCACA
C[CG]GGCACTGGGC[[CG]]CCACCTTTGTTGATGGAAGAGAGA
AACTGAGGCATAGTGCCCAGGGCCAGGGGAC[[CG]]CAACCACC
[CG]GCCCTTGTCACTGTAG[CG]TTAAGGCTCAGAGTCTG[CG]
CAGCAGATATGTGTC[CG]CACC[CG]CCAGGCCTTCAGCTTCCC
TCAGACAGG[CG]GGAAAACCCTAGGCTGCCCTTCTC[CG]AG[C
G]GAGGGC[CG]CCCCACACACAGCAGG[CG]CTTAAA[CG]GGT
A[CG][CG]GGGCCCTGGA[CG]GCTCTC[CG][CG]GAGCTCCC
CAGGCTCTGG[CG]CAGTTCCC[CG]GCTTGGGGACCTGAGCAC[
CG]CCTCTGCCTGCCCCAGCTGCTCACCTCCCCTTTCCCACAGAG
CAGGGCCAGC[CG]T
SEQ ID NO: 5311
TTGGGGT[CG]CTCTCTGACCCC[CG][CG]CTGCATCCC[CG]G
GAC[CG]ACCCACCTGCTG[CG]CAGCTACCCCAGGC[CG]GTCC
CAGC[CG]CC[CG]GAGCCCCAGTG[CG][CG]ATGG[CG]GC[[
CG]]GCAAACTG[CG]CCTG[[CG]]CACTGGGCCTCAC[CG][C
G]GACTA[CG]ACTCCCACAATGC[CG][CG]AGGCTGTGC[CG]
[CG]CAC[CG]GGG[CG]AGCAAGGGAGC[CG]TGACAGGTGACA
GTG[CG]CATGGCCAATCAGA[CG][CG]AGTGTGGCCTCC[[CG
]]G[CG]TCC[CG]CCCACTC[CG]G[[CG]]CCTG[CG]TCATT
TTGGGCACCATGGCAA[CG]AAGTAAACAAG[[CG]]GGTAAATA
ATAGACCCCTGACAGCCAGAGACC[CG]GAGAAACAAAGA[CG]G
TGAGAAA[CG]GAGAAACAAAGACAAAAAGA[CG][CG]CAGAC[
CG]GGAGGGAGAGAAGCATGAAGGCCCACAGA[CG]CT[CG]GGG
GCAGACCAAGA[CG]CCCTGGGGTCCC[[CG]]AAACAGAGATGT
ACAGAGT[[CG]]GACA[[CG]]GAAAGGGAGAGACCAGCTCA[C
G]GTTAAGAAAGGAGGGAGACCAGCCCAGGATCAAGAAAGGGAAA
GACCAGCATGGGCTCAAGAAAAGAAAGAGACCAGCTCAG
SEQ ID NO: 5312
CTGACCCC[CG][CG]CTGCATCCC[CG]GGAC[CG]ACCCACCT
GCTG[CG]CAGCTACCCCAGGC[CG]GTCCCAGC[CG]CC[CG]G
AGCCCCAGTG[CG][CG]ATGG[CG]GC[CG]GCAAACTG[CG]C
CTG[CG]CACTGGGCCTCAC[CG][CG]GACTA[CG]ACTCCCAC
AATGC[CG][CG]AGGCTGTGC[CG][CG]CAC[CG]GGG[CG]A
GCAAGGGAGC[CG]TGACAGGTGACAGTG[CG]CATGGCCAATCA
GA[CG][CG]AGTGTGGCCTCC[[CG]]G[CG]TCC[CG]CCCAC
TC[CG]G[[CG]]CCTG[[CG]]TCATTTTGGGCACCATGGCAA[
[CG]]AAGTAAACAAG[[CG]]GGTAAATAATAGACCCCTGACAG
CCAGAGACC[CG]GAGAAACAAAGA[CG]GTGAGAAA[CG]GAGA
AACAAAGACAAAAAGA[CG][CG]CAGAC[CG]GGAGGGAGAGAA
GCATGAAGGCCCACAGA[CG]CT[CG]GGGGCAGACCAAGA[CG]
CCCTGGGGTCCC[[CG]]AAACAGAGATGTACAGAGT[[CG]]GA
CA[[CG]]GAAAGGGAGAGACCAGCTCA[CG]GTTAAGAAAGGAG
GGAGACCAGCCCAGGATCAAGAAAGGGAAAGACCAGCATGGGCTC
AAGAAAAGAAAGAGACCAGCTCAGGCTCAGGAAAAGA
SEQ ID NO: 5313
GAGTCTGAAG[CG]CC[CG][CG]AGGAAG[CG]AG[CG]CCAG[
CG][CG]GGC[CG]C[CG]G[CG]ATGA[CG]GC[CG][CG]AAG
CAGGAGC[CG]CAGCCCACCC[CG]GGGGCCAGGG[CG]AGCCAG
G[CG]CAGC[CG]G[CG]GACCAGGTGAGAGT[CG]GCAGC[CG]
[CG]GCCAGGCCCTCC[CG]GGAGGGGTGGCTCCAGTG[CG][CG
]CTC[CG]CC[CG]CCTCC[CG]CTTCCCAGGCTGGGCTCC[CG]
[CG]CCTCCCTCTTCTCACCCTCCCC[CG]CCC[CG]CCCCAGTT
CCAGGCTCTCCTGCTTCTCCA[CG]GACTCTG[CG]GGAAGTTAG
AGCCTCTG[CG]TG[CG]CTC[CG]GGGCC[CG]G[CG]AGAGGA
TG[CG]CAAGGTGGAGAGC[[CG]][CG]GGGAAGGGGGCAGAGA
GGTAAAGGCTGAAGGTGCCC[CG]GGGAACCC[CG]G[CG]GG[C
G]GCCCAC[CG]AGGGAGGGAGAGG[CG]GC[CG]GGACCAAGGA
ATGGGGCCTCTTGGTTCCCCATTAA[[CG]]CA[CG]CTGAAGAA
ATCTGCTG[CG]CTCCTGA[CG]GC[CG]CTCAC[CG]GGTT[CG
]AGCCC[CG]TCCTCCTATAGC[CG]GGG[CG]CT[CG]CTGGCC
AAAG[CG]ACC[CG]AGCAGG[CG]AATGACCTTTAGG[CG]GA[
CG]GGGTTTTCCCT
SEQ ID NO: 5314
GACTGGAAC[CG][CG]GCC[CG]AGGTAACTGGAGGGCA[CG]G
[CG]GGGG[[CG]]GGG[[CG]]CTCATCCCAGG[CG]GCTTCTG
ATTGGCCT[CG]GC[CG]CCT[CG]TG[CG]TCACAGCCC[CG]C
CCCTGCC[CG]GGAGTCC[CG]CAG[CG]GTGACTTGTCAC[CG]
A[CG]CC[CG]G[CG]TGATCCAG[CG]GACCAATGG[CG]GC[C
G]CC[CG][CG]G[CG][CG]CAGGGTG[CG]GGGATGGGGTC[C
G]CC[CG][CG]G[CG]TTGGG[CG][CG]CT[CG]CCAGCCCAG
GGGT[CG]CCATGAC[CG]AGTGGCCCAGGCC[CG]AG[CG]AAG
CC[CG][CG][CG][CG]GTGAGTC[CG]C[CG][CG]GCCCATC
[CG]TCCCTC[CG]CC[CG]CCAGAG[CG]TCCAT[CG]GGA[CG
]CCCACC[CG]GGAGGGTCT[CG]G[CG]C[CG][CG]T[CG]G[
CG]GGAGCTGGCC[CG]GGAT[CG][CG]GTCCAGGGG[CG][CG
][CG]T[CG]GGGGAGG[CG]GGCC[CG]GGCC[CG]GGAGGGGG
[CG]GGC[CG]GGC[CG]GGGTTCCAGAGCCCAT[CG]GGAGTCC
TGAGGT[[CG]]C[CG]TAGGGGAGGGAAGGGGAAGGCTAGAGGC
CA[CG][CG]GGG[CG]GGGACTGGGGGGGCTGGGGC[CG]CCCC
TTC[CG]CAGGGACCC[CG]ACCCCTGTTGGCATC[CG]CCTGTC
[CG]TTT
SEQ ID NO: 5315
A[CG][CG]GGCTTCACTACTAG[CG]TGGTGC[[CG]]C[CG]C
TGCTGGCCAACCTG[CG][CG]GACCAG[CG]CTCTGGCTGC[CG
][CG]CAGCCACTGCA[CG]GCCCAGCTGTG[CG]CAT[CG]CTG
GCTCTGGGTTC[CG]C[CG]AATG[CG]TCCTCCTGG[CG]GTGA
TGGCTCTGGAC[CG][CG][CG]GC[CG]CAGTGTGC[CG]CC[C
G]CTG[CG]CTATG[CG]GGGCT[CG]TCTCCC[CG][CG]CCTA
TGT[CG]CA[CG]CTGGCCAG[CG]CCTCCTGGCTAAG[CG]GCC
TCACCAACT[CG]GTTG[CG]CAAAC[CG][CG]CTCCTGGCTGA
G[CG]GC[CG]CTGTG[CG][CG]CCC[CG]CCTGCTGGACCACT
TCATCTGTGAGCTGC[CG]G[CG]TTGCTCAAGCTGGCCTG[CG]
GAGG[CG]A[[CG]]GAGACACTAC[CG]AGAACCAGATGTT[[C
G]]C[CG]CC[CG][CG]TGGTCATCCTGCTGCTGC[CG]TTTGC
[CG]TCATCCTGGCCTCCTA[CG]GTGC[CG]TGGCC[CG]AGCT
GTCTGTTGCATG[CG]GTTCAG[CG]GAGGC[[CG]]GAGGAGGG
[CG]GTGGGCA[CG]TGTGGGTCCCACCTGACAGC[CG]TCTGCC
TGTTCTA[[CG]]GCT[[CG]]GCCATCTACACCTACCTGCAGCC
[CG][CG]CAG[CG]CTACAACCAGG
SEQ ID NO: 5316
[CG]G[CG]ATGA[CG]GC[CG][CG]AAGCAGGAGC[CG]CAGC
CCACCC[CG]GGGGCCAGGG[CG]AGCCAGG[CG]CAGC[CG]G[
CG]GACCAGGTGAGAGT[CG]GCAGC[CG][CG]GCCAGGCCCTC
C[CG]GGAGGGGTGGCTCCAGTG[CG][CG]CTC[CG]CC[CG]C
CTCC[CG]CTTCCCAGGCTGGGCTCC[CG][CG]CCTCCCTCTTC
TCACCCTCCCC[CG]CCC[CG]CCCCAGTTCCAGGCTCTCCTGCT
TCTCCA[CG]GACTCTG[CG]GGAAGTTAGAGCCTCTG[[CG]]T
G[CG]CTC[CG]GGGCC[CG]G[CG]AGAGGATG[CG]CAAGGTG
GAGAGC[[CG]][CG]GGGAAGGGGGCAGAGAGGTAAAGGCTGAA
GGTGCCC[CG]GGGAACCC[CG]G[CG]GG[CG]GCCCAC[CG]A
GGGAGGGAGAGG[CG]GC[CG]GGACCAAGGAATGGGGCCTCTTG
GTTCCCCATTAA[CG]CA[CG]CTGAAGAAATCTGCTG[CG]CTC
CTGA[CG]GC[CG]CTCAC[CG]GGTT[CG]AGCCC[CG]TCCTC
CTATAGC[CG]GGG[CG]CT[CG]CTGGCCAAAG[CG]ACC[CG]
AGCAGG[CG]AATGACCTTTAGG[CG]GA[CG]GGGTTTTCCCTC
TGCTTTCTTGTTTCTTTTGAGGAGA[CG]GGTGTGTGTTTGTGAG
SEQ ID NO: 5317
AGA[CG][CG]GGCTTCACTACTAG[CG]TGGTGC[CG]C[CG]C
TGCTGGCCAACCTG[CG][CG]GACCAG[CG]CTCTGGCTGC[CG
][[CG]]CAGCCACTGCA[CG]GCCCAGCTGTG[CG]CAT[CG]C
TGGCTCTGGGTTC[CG]C[CG]AATG[CG]TCCTCCTGG[CG]GT
GATGGCTCTGGAC[[CG]][CG][CG]GC[CG]CAGTGTGC[CG]
CC[CG]CTG[CG]CTATG[CG]GGGCT[CG]TCTCCC[CG][CG]
CCTATGT[CG]CA[CG]CTGGCCAG[CG]CCTCCTGGCTAAG[CG
]GCCTCACCAACT[CG]GTTG[CG]CAAAC[CG][CG]CTCCTGG
CTGAG[CG]GC[CG]CTGTG[CG][CG]CCC[CG]CCTGCTGGAC
CACTTCATCTGTGAGCTGC[CG]G[CG]TTGCTCAAGCTGGCCTG
[CG]GAGG[CG]A[[CG]]GAGACACTAC[CG]AGAACCAGATGT
T[CG]C[CG]CC[CG][[CG]]TGGTCATCCTGCTGCTGC[CG]T
TTGC[[CG]]TCATCCTGGCCTCCTA[CG]GTGC[CG]TGGCC[C
G]AGCTGTCTGTTGCATG[CG]GTTCAG[CG]GAGGC[[CG]]GA
GGAGGG[CG]GTGGGCA[CG]TGTGGGTCCCACCTGACAGC[CG]
TCTGCCTGTTCTA[[CG]]GCT[CG]GCCATCTACACCTACCTGC
AGCC[[CG]][[CG]]CAG[[CG]]CTACAACCA
SEQ ID NO: 5318
TGTGCTG[CG]CTGGC[CG]CCTGGACCAGGGTGGCTGCTGGAGA
GTGTTTTCCCTCTGGGTTTCCTTCTCCTTGGCTCCCAGGGAATCC
TCTGG[[CG]]GTGCCTC[CG]GGAGGCTGGTAGC[CG]GGGAAG
CAGGCA[[CG]]TGAATCCAC[CG]GGCTGCCCCACCCCAACCTG
AGGTCAGAGCTGAGG[CG]TAGGGGC[CG]GGGTAGGGGCAGAGG
TGC[CG]AAA[CG]CACTAGCTGGGGACTCC[CG]GGGGAAGGAG
ATAGGAGAGACCCAAGG[CG]AGCCTTAGGACT[CG]CCCCTATG
ACTATGACTA[CG][CG]CTG[[CG]]TC[CG]AACCACATGTCC
ACCC[CG]CATTTGCCTT[CG]GGGACCAGAC[CG][CG]CTCCC
TCAGATGGCTGGGGAGGGAGCAGACTC[CG]GGTGAAGGCTGGGG
GAGGTAC[CG]GGATGC[CG]AGTA[CG]CCAGAGCAGG[CG]GG
GGATGGGTTC[[CG]]GCTTCCTGC[CG]CCT[CG]GCATCT[CG
]CTTTGCAC[CG]GGCAAAGAAGGGGCCA[CG]AC[CG]G[CG]A
AGAG[CG][CG]TGGAGACACAGGGACC[CG][CG]ATCCAGGGG
CAGGAACCC[CG]CCCCCTCC[CG]GAAACCTCC[CG]GGCCCTG
AGT[CG]TGCC[CG]G[CG]CTC
SEQ ID NO: 5319
C[CG][CG][CG][CG]T[CG]GGGCTGCTCC[CG]GGAGGAAGG
[CG]G[CG][CG]GAGGC[CG]GGGG[CG]GC[CG]CTGAGCTTG
G[CG]TC[CG][CG][CG]GCTC[CG]GTG[CG]GGCTG[CG]C[
CG][CG]CCTTCCCCAG[CG]AGCTAC[CG]AGCTTGGGGC[CG]
C[CG][CG]GTC[CG]CTCC[CG]C[CG]CC[CG]GCCTCTCCCT
CCT[CG]GCCAC[CG]C[CG]CAGCCCCTGCCC[CG]C[CG]AGC
CC[CG]C[CG]GACAG[CG]G[CG]GC[CG]CAG[CG][CG]CAT
TTGGGCTC[CG]AGGAAGTTGAC[CG]AGG[CG]GCTGC[CG]CA
GGATCC[CG]GGCC[CG]GAT[CG]CA[CG]AAGCC[CG][CG][
[CG]]GC[CG]TCTCCTC[CG][CG][CG]CCACCCCTG[CG]CC
TCC[CG][CG]AGCTCCACTTCCCATCTGCTATTGTTTC[CG]AT
TGTTTTC[CG]GTGG[[CG]]AGCC[CG]GCTC[CG]AAACTTAC
AAAGTGTTGGATGTCCCC[CG]TT[CG]AACTGAGGGACTGCAGA
C[CG]CCTCTGGGTAGCTGGATGAAGCCCACCC[CG]TCCCCTTC
TGGTACCAAAGTGCTTACTCCTCTCCAAAGTGC[CG]TGTCTGAA
CTGC[CG]CTGGGAAGAAG[CG]GCTCCTGAGA[CG][CG]CCCA
CACCTTTCACCTGC[CG][CG][CG]CTTCCCCCTCCT
SEQ ID NO: 5320
C[CG]CC[CG]GCCTCTCCCTCCT[CG]GCCAC[CG]C[CG]CAG
CCCCTGCCC[CG]C[CG]AGCCC[CG]C[CG]GACAG[CG]G[CG
]GC[CG]CAG[CG][CG]CATTTGGGCTC[CG]AGGAAGTTGAC[
CG]AGG[CG]GCTGC[CG]CAGGATCC[CG]GGCC[CG]GAT[CG
]CA[CG]AAGCC[CG][CG][[CG]]GC[CG]TCTCCTC[CG][C
G][CG]CCACCCCTG[CG]CCTCC[CG][CG]AGCTCCACTTCCC
ATCTGCTATTGTTTC[CG]ATTGTTTTC[CG]GTGG[[CG]]AGC
C[CG]GCTC[CG]AAACTTACAAAGTGTTGGATGTCCCC[CG]TT
[CG]AACTGAGGGACTGCAGAC[CG]CCTCTGGGTAGCTGGATGA
AGCCCACCC[CG]TCCCCTTCTGGTACCAAAGTGCTTACTCCTCT
CCAAAGTGC[CG]TGTCTGAACTGC[CG]CTGGGAAGAAG[CG]G
CTCCTGAGA[CG][CG]CCCACACCTTTCACCTGC[CG][CG][C
G]CTTCCCCCTCCT[CG]GCCACCTTCC[CG]G[CG]GAAGCAG[
CG]AGGAGGGAGCCCCCTTTGGC[CG]TCCTC[CG]TGGAAC[CG
]GTTTTC[CG]AGGCTGGCAAAAGC[CG]AGGCTGGATTTGGGGG
AGGAATATTAGACT[CG]GAGGAGTCTG[CG][CG]CTTTTCTCC
TCCC
SEQ ID NO: 5321
GC[CG]CC[CG]GCAGGGAGAGG[CG]GAGTCACTGG[CG]CC[C
G]GGG[CG]GGGTGTTGGGGAGGGGTGAGTAGGGACAGGAGGGAG
GGGGTGGAGAGTTGGGCTGCTG[CG]A[CG]CCTCC[CG]GTGGC
TCTGG[CG]GCACCAATG[CG]GGGCAC[CG]GAGCAGGTGTC[C
G]ACTG[[CG]]GTG[CG]GGCTACCACTC[CG]C[CG]GGGAAC
CT[CG]TCTTGCTCCTCCCTG[CG]CTT[CG]CCTCTGGAATCTC
TCCCTTATCTCATGC[CG][CG]GTGAAACTTTGCTCAAGTTGGT
GGTGCTCTGAGGTCTCCAGGGCTGGAGGC[CG][CG][CG]A[CG
][CG]AAT[CG]C[CG]ACACTGGATTCT[CG]GG[CG]TTAGTC
[CG]AAGTTTAAGTTCCCTT[CG]TTCACTCCTTTGCCCTGGAT[
[CG]]AAC[CG]CAGCCCCAGGA[CG]CAG[CG]GGTTCCACTGG
AAA[CG]GGGAAT[CG]GGGAGAGG[CG]GT[CG]CTGGGCTC[C
G]GG[CG]ACCTGGGAA[CG]AGGG[CG]TTTGCTCCCCCTCC[C
G]AC[CG]AG[CG]GG[CG]GC[CG]G[CG]TCAGCAC[CG][CG
]GACAG[CG]CAT[CG]GTGC[CG]CAGC[CG][CG]GG[CG]GG
[CG]C[CG]CTAGGGCTGCCAGGGGTC[CG]AGG[CG][[CG]]G
GCAAGGCTGCTCAC
SEQ ID NO: 5322
GTGGAGAGTTGGGCTGCTG[CG]A[CG]CCTCC[CG]GTGGCTCT
GG[CG]GCACCAATG[CG]GGGCAC[CG]GAGCAGGTGTC[CG]A
CTG[[CG]]GTG[CG]GGCTACCACTC[CG]C[CG]GGGAACCT[
CG]TCTTGCTCCTCCCTG[CG]CTT[CG]CCTCTGGAATCTCTCC
CTTATCTCATGC[CG][CG]GTGAAACTTTGCTCAAGTTGGTGGT
GCTCTGAGGTCTCCAGGGCTGGAGGC[CG][CG][CG]A[CG][C
G]AAT[CG]C[CG]ACACTGGATTCT[CG]GG[CG]TTAGTC[CG
]AAGTTTAAGTTCCCTT[CG]TTCACTCCTTTGCCCTGGAT[[CG
]]AAC[CG]CAGCCCCAGGA[CG]CAG[CG]GGTTCCACTGGAAA
[CG]GGGAAT[CG]GGGAGAGG[CG]GT[CG]CTGGGCTC[CG]G
G[CG]ACCTGGGAA[CG]AGGG[CG]TTTGCTCCCCCTCC[CG]A
C[CG]AG[CG]GG[CG]GC[CG]G[CG]TCAGCAC[CG][CG]GA
CAG[CG]CAT[CG]GTGC[CG]CAGC[CG][CG]GG[CG]GG[CG
]C[CG]CTAGGGCTGCCAGGGGTC[CG]AGG[CG][CG]GGCAAG
GCTGCTCACCC[CG]CTGTGCTGTGCACCCCAAACCTTCCCTGTT
TTCCTTTCTACCTCC[CG]TGTGCACCCCCCTACC[CG]CCACC[
CG]AGGCA
SEQ ID NO: 5323
TG[CG]GGCTG[CG]C[CG][CG]CCTTCCCCAG[CG]AGCTAC[
CG]AGCTTGGGGC[CG]C[CG][CG]GTC[CG]CTCC[CG]C[CG
]CC[CG]GCCTCTCCCTCCT[CG]GCCAC[CG]C[CG]CAGCCCC
TGCCC[CG]C[CG]AGCCC[CG]C[CG]GACAG[CG]G[CG]GC[
CG]CAG[CG][CG]CATTTGGGCTC[CG]AGGAAGTTGAC[CG]A
GG[CG]GCTGC[CG]CAGGATCC[CG]GGCC[CG]GAT[CG]CA[
CG]AAGCC[CG][CG][[CG]]GC[CG]TCTCCTC[CG][CG][C
G]CCACCCCTG[CG]CCTCC[CG][CG]AGCTCCACTTCCCATCT
GCTATTGTTTC[CG]ATTGTTTTC[CG]GTGG[CG]AGCC[CG]G
CTC[CG]AAACTTACAAAGTGTTGGATGTCCCC[CG]TT[CG]AA
CTGAGGGACTGCAGAC[CG]CCTCTGGGTAGCTGGATGAAGCCCA
CCC[CG]TCCCCTTCTGGTACCAAAGTGCTTACTCCTCTCCAAAG
TGC[CG]TGTCTGAACTGC[CG]CTGGGAAGAAG[CG]GCTCCTG
AGA[CG][CG]CCCACACCTTTCACCTGC[CG][CG][CG]CTTC
CCCCTCCT[CG]GCCACCTTCC[CG]G[CG]GAAGCAG[CG]AGG
AGGGAGCCCCCTTTGGC[CG]TCCTC[CG]TGGAAC[CG]GTTTT
C[CG]AGGCTGGCAAAAG
SEQ ID NO: 5324
GCC[CG]AGCTGGG[CG]C[CG]TCCTAGAGCCAAG[CG]CC[CG
]CTAGAGC[CG]AGACTAGGGGG[CG]GTGAGGAGTGGGACC[CG
]CT[CG][CG]GCCATTGGTTGTG[CG]GCCAGGAGGGG[CG]GA
GGGA[CG]A[CG][CG][CG]GGCTGTG[CG]GGGC[CG]GCAGC
C[CG]GATCCACTCC[CG]CTG[CG]CCTGCTGTC[CG]C[CG]T
G[CG]CCCAGACTG[CG][CG]C[CG][CG]C[CG]CTG[CG]CC
CAACATTCC[CG]AGGA[CG]GCTT[CG][CG]GG[CG][CG]TA
T[CG]TCCAGAC[CG]GAGCAC[CG]CCCCAC[CG]CTAG[CG]C
AGGAGACCTGC[CG]GGGAAGT[CG][[CG]]TGTCCTGACCTGC
AGGTCAGTGAGGGAAACTTGCAGGGTCC[CG]AGCTCTGGGGGTG
AACTTGCCCTA[CG][CG]GAGAG[CG]CCCCACTCCTTGCCTGG
GG[CG]GCCT[CG]GGTCCTGGAGG[[CG]]AGC[CG]CCTCCCA
GACTCTCCCC[CG]AGATCTGGGCTCC[CG]GAGATGTGGCTCCT
TTCTGAGAAACACCCCCAAATCTATCCCTTTTGAGGTGTCCTTAC
TGTCC[CG]AGAAGCAAT[CG]CAGCC[CG]GCTCTC[CG]AGTC
TTGGGGTGACTCCA[CG]GGCTGCAGGAGGAGC[CG][CG]AGGG
GCCCTG
SEQ ID NO: 5325
GAGGGCAGGGC[CG]CAAGACTTCTGGGGTC[CG]AATAGGTCAG
GGGT[CG]GCAGCCCCAG[CG]GGTG[CG]TAGCCAGAGCTTGGA
CT[CG]GGGAAGAGGG[CG]AGAAT[CG]TGACCTCAAGAAGCTG
GGGGGGGGTGGGGTGGCCAGGGGGAAGGGAGGGGAGG[CG]GTGG
GGG[CG]CCCTTGTGGGTAGGTGGGATG[CG]AAGGGGAGGAGGG
GTCTAGAAC[CG]CCTG[CG]CCT[CG][CG]GTTTCCTGCAGGT
GAGCCC[CG]AGGGAG[CG]GGGGA[CG]AGGACT[CG]TGCTCC
TCCT[CG]GCCC[CG]CTGTCCC[CG]T[[CG]]TCCT[CG]CCC
[CG]GTCCATGGCCT[CG]GGCTC[CG]GCTGCCCTCCTGGCAAG
TGTGTGTGCAACAGTTG[CG]GCCTGGAGAT[[CG]]TGGACAAA
TACCTTCTCAAGGTAGGATAGGGCCT[CG]GGTG[CG]AGGCCCA
AGGGGAG[CG]GGCTTAG[CG]TGGGAGCTCTGGGAAAAGAGTAA
TGTTCCTCCCACCAACCTTTC[CG]TTCAAGTTCTGGGTGAAGGT
TGTAGGCTTTTGCAGAAAATCAGCCCAGTTTAGGGCAACTCATG[
CG]GTGGGATAAAGTGAGAAGAAATCCCAGACTCTTCTATGTC
SEQ ID NO: 5326
CAAGCAGCTGGTAGGAGAATCTGGGCTGGATT[CG]GAAGG[CG]
CAGCTACAGGA[CG]CC[CG]CTGCCC[CG]GAT[CG]GAAGG[C
G]CAGCACCTCAGGCCACAATCTCAGACA[CG]CCCT[CG]GGTC
C[CG]ACAGGTGTTGCC[CG]CCTCCCA[CG]GGGCTC[CG]GGA
CCC[CG]GTCCC[CG]GGCTGCCCCCTGCCTTCTGGGG[CG]CTG
GAG[CG][CG][CG]CAGTGG[CG]GAGCTGGAGCCACCCAGTGC
TT[CG]CA[CG]GG[CG][CG]CTC[CG]CTTCTC[CG]GGTTTT
AG[CG]GAAGCCTG[CG]GGGGG[CG]GGGTAAC[CG][CG]GAA
GC[[CG]]G[CG]GC[[CG]]TGGG[CG][CG][CG]GGTTGGGG
GCTCT[CG][CG]C[CG]CTC[CG]GGCTCTCCCCCCCCC[CG]G
CTG[CG]GTTGC[CG]AAGAGAGGC[CG]GAG[CG][CG]G[CG]
CC[CG]GCTGCTCCACCTGG[CG][CG]CTGAGCAC[CG][CG][
CG]GGGAGCCCC[CG]GGGG[CG]GG[CG]GTGGGGGG[CG][CG
]GACTG[CG]GGAAGAAGGCA[CG]AGG[CG]T[CG]C[CG]CAG
CT[CG]GTCAGGGGC[CG]GGGCCC[CG]C[CG]CT[CG]C[CG]
CTGCACTAACTTGC[CG]CTTGGTCT[CG]CCT[CG]C[CG]C[C
G][CG]GCT[CG]CTG[CG]CTTTGGGTGG[CG]GGGGG[CG]G
SEQ ID NO: 5327
GCAGCTGGTAGGAGAATCTGGGCTGGATT[CG]GAAGG[[CG]]C
AGCTACAGGA[CG]CC[CG]CTGCCC[CG]GAT[[CG]]GAAGG[
CG]CAGCACCTCAGGCCACAATCTCAGACA[CG]CCCT[CG]GGT
CC[CG]ACAGGTGTTGCC[CG]CCTCCCA[CG]GGGCTC[CG]GG
ACCC[CG]GTCCC[CG]GGCTGCCCCCTGCCTTCTGGGG[CG]CT
GGAG[CG][CG][CG]CAGTGG[CG]GAGCTGGAGCCACCCAGTG
CTT[CG]CA[CG]GG[CG][CG]CTC[CG]CTTCTC[CG]GGTTT
TAG[CG]GAAGCCTG[CG]GGGGG[CG]GGGTAAC[CG][CG]GA
AGC[[CG]]G[[CG]]GC[[CG]]TGGG[CG][CG][CG]GGTTG
GGGGCTCT[CG][CG]C[CG]CTC[CG]GGCTCTCCCCCCCCC[C
G]GCTG[CG]GTTGC[CG]AAGAGAGGC[CG]GAG[CG][CG]G[
CG]CC[CG]GCTGCTCCACCTGG[CG][CG]CTGAGCAC[CG][C
G][CG]GGGAGCCCC[CG]GGGG[CG]GG[CG]GTGGGGGG[CG]
[CG]GACTG[CG]GGAAGAAGGCA[CG]AGG[CG]T[CG]C[CG]
CAGCT[CG]GTCAGGGGC[CG]GGGCCC[CG]C[CG]CT[CG]C[
CG]CTGCACTAACTTGC[CG]CTTGGTCT[CG]CCT[CG]C[CG]
C[CG][CG]GCT[CG]CTG[CG]CTTTGGGTGG[CG]GGGGG[CG
]GGGA
SEQ ID NO: 5328
TCCCTGCAGC[CG]GTCCTCTT[CG]CCCTTGTCCTCCTGTGCTA
CCTCCTGACCTTGA[CG]GGCAACT[CG]G[CG]CTGGTGCTGCT
GG[[CG]]GTG[[CG]][CG]ACC[CG][CG]CCTGCACA[CG]C
CCATGTACTACTTCCTCTGCCACCTGGCCTTGGTAGA[CG][CG]
GGCTTCACTACTAG[[CG]]TGGTGC[CG]C[CG]CTGCTGGCCA
ACCTG[CG][CG]GACCAG[CG]CTCTGGCTGC[CG][[CG]]CA
GCCACTGCA[CG]GCCCAGCTGTG[CG]CAT[CG]CTGGCTCTGG
GTTC[CG]C[CG]AATG[CG]TCCTCCTGG[CG]GTGATGGCTCT
GGAC[[CG]][CG][CG]GC[CG]CAGTGTGC[CG]CC[CG]CTG
[CG]CTATG[CG]GGGCT[CG]TCTCCC[CG][CG]CCTATGT[C
G]CA[[CG]]CTGGCCAG[CG]CCTCCTGGCTAAG[CG]GCCTCA
CCAACT[CG]GTTG[CG]CAAAC[CG][CG]CTCCTGGCTGAG[C
G]GC[CG]CTGTG[[CG]][[CG]]CCC[[CG]]CCTGCTGGACC
ACTTCATCTGTGAGCTGC[CG]G[CG]TTGCTCAAGCTGGCCTG[
CG]GAGG[CG]A[[CG]]GAGACACTAC[CG]AGAACCAGATGTT
[[CG]][[CG]]CC[[CG]][[CG]]TGGTCATCCTGCTGCTGC[
[CG]]TTTGC[[CG]]TCATCCTGGCCTCCTA[CG]GTGCC
SEQ ID NO: 5329
AGAAGGAGCACTGCTGTC[CG]CCCCAAGAA[CG]GGGTCTGTC[
CG]CC[CG]AGCTCTAAGG[CG]AGCATCTTCAGGGGGACAGTTT
CTG[CG]AAGG[CG]GGGAGAGGAGTCCCTCCCCAACTTCACTCC
CCTAATCAAGC[CG][CG]GGTGAGATAGGCAGC[CG]G[CG]GT
TTGTGCCCATCTGTGGGGGCAAGTCCTACCTTCTGCCAAAAGG[C
G][CG]A[CG]CATGCACAAAAGATGGATCCAGGCTATCTTTCTC
TGCCATCAGCTCAGGCAAATATTTCTCCTCTTCCATAG[CG][CG
]GACTT[CG]GATTGTCCC[[CG]]GG[CG]AAG[CG][CG]AGG
TTC[CG]CT[CG]CT[CG]GA[CG]CAGGCAGGGTCTTGGGGCAG
[CG]CCTGGCTCC[CG][CG]CTGCTCCTCCTC[CG][CG][CG]
G[CG]AGGGATCTCTGTG[CG]TCCTCACTGGCCCATGCACCCAG
CACCTG[CG]ACTCC[CG]C[CG]T[CG]GGCTG[CG]TGGCCC[
CG][CG]CCCACACCTGCC[CG]TCCCTTC[CG]T[CG]TCCCT[
CG]CT[CG][CG]CAGAGCCC[CG]GCTCACACCAG[CG]GCCTT
AACTGGAGAGG[[CG]]GGAACAGGA[CG][CG]GCCCACCTC[C
G]CCAACCACTCATTGGTGGTGGTGCCAGGAGA
SEQ ID NO: 5330
[CG]CAAGACTTCTGGGGTC[CG]AATAGGTCAGGGGT[CG]GCA
GCCCCAG[CG]GGTG[CG]TAGCCAGAGCTTGGACT[CG]GGGAA
GAGGG[CG]AGAAT[CG]TGACCTCAAGAAGCTGGGGGTGGGGTG
GGGTGGCCAGGGGGAAGGGAGGGGAGG[CG]GTGGGGG[CG]CCC
TTGTGGGTAGGTGGGATG[CG]AAGGGGAGGAGGGGTCTAGAAC[
CG]CCTG[CG]CCT[CG][CG]GTTTCCTGCAGGTGAGCCC[CG]
AGGGAG[CG]GGGGA[CG]AGGACT[CG]TGCTCCTCCT[CG]GC
CC[CG]CTGTCCC[CG]T[CG]TCCT[CG]CCC[[CG]]GTCCAT
GGCCT[CG]GGCTC[CG]GCTGCCCTCCTGGCAAGTGTGTGTGCA
ACAGTTG[CG]GCCTGGAGAT[[CG]]TGGACAAATACCTTCTCA
AGGTAGGATAGGGCCT[CG]GGTG[CG]AGGCCCAAGGGGAG[CG
]GGCTTAG[CG]TGGGAGCTCTGGGAAAAGAGTAATGTTCCTCCC
ACCAACCTTTC[CG]TTCAAGTTCTGGGTGAAGGTTGTAGGCTTT
TGCAGAAAATCAGCCCAGTTTAGGGCAACTCATG[CG]GTGGGAT
AAAGTGAGAAGAAATCCCAGACTCTTCTATGT[CG]TTGCAAAAC
A
SEQ ID NO: 5331
G[CG]GTCACACACCTGCAGGAACA[CG][CG]CACACTACACAC
[CG]TTGCTCT[CG]CCTCC[CG]GTATCACCTT[CG]AA[CG][
CG]CTGGC[CG]GCCCCCTCCTAGCCTGGTGCAGT[CG]GCTC[[
CG]]CTTCTACTCAGTCT[CG]GGTCCTCCTGGGCCAA[CG]CAG
ACCCTGGTGCCTCCTTCCTTAGA[CG]AGCTAAGGTCAAGACTTC
TGCTGCTG[CG]GAGGAGCC[CG][[CG]]CACCAGGC[CG]GGG
TGGACTG[CG]CCTG[CG]CAT[CG]GGCC[CG]CCTGGTTCTTA
GGAACTA[CG]ACTCCCAGGGGG[CG]CTG[CG][CG]CT[[CG]
][[CG]]GCAC[CG][CG]ACTAAAGCTTGCAAAGCCTGGGTCAT
[[CG]]AAACTACAACTCCCAGAAGGCTAAG[[CG]]TCTGCACA
[CG]CTAGGTC[CG]CAGGG[CG]CTGACCCCTGGGGGCC[CG]A
GGAG[CG]C[CG]GGCTGCTGCCC[CG]AGCCC[CG]CCCACTAC
AGCCTCAGTGCATGG[CG]GTGGG[CG]CTTCCAGTAACTCAGGG
GTGGGACTTTTTTCTTGCTCAGGGGATGAGGC[CG]AGCCCACAG
GCTTAGAAAGAAGGAACTTGAGTC[CG]GGCACCTGTCATCCCAG
[CG]CTTAGGGAGGC[CG]AGG[CG]GGTGGA
SEQ ID NO: 5332
CTGGTAGGAGAATCTGGGCTGGATT[CG]GAAGG[CG]CAGCTAC
AGGA[CG]CC[CG]CTGCCC[CG]GAT[[CG]]GAAGG[CG]CAG
CACCTCAGGCCACAATCTCAGACA[CG]CCCT[CG]GGTCC[CG]
ACAGGTGTTGCC[CG]CCTCCCA[CG]GGGCTC[CG]GGACCC[C
G]GTCCC[CG]GGCTGCCCCCTGCCTTCTGGGG[CG]CTGGAG[C
G][CG][CG]CAGTGG[CG]GAGCTGGAGCCACCCAGTGCTT[CG
]CA[CG]GG[CG][CG]CTC[CG]CTTCTC[CG]GGTTTTAG[CG
]GAAGCCTG[CG]GGGGG[CG]GGGTAAC[CG][CG]GAAGC[[C
G]]G[[CG]]GC[CG]TGGG[CG][CG][CG]GGTTGGGGGCTCT
[CG][CG]C[CG]CTC[CG]GGCTCTCCCCCCCCC[CG]GCTG[C
G]GTTGC[CG]AAGAGAGGC[CG]GAG[CG][CG]G[CG]CC[CG
]GCTGCTCCACCTGG[CG][CG]CTGAGCAC[CG][CG][CG]GG
GAGCCCC[CG]GGGG[CG]GG[CG]GTGGGGGG[CG][CG]GACT
G[CG]GGAAGAAGGCA[CG]AGG[CG]T[CG]C[CG]CAGCT[CG
]GTCAGGGGC[CG]GGGCCC[CG]C[CG]CT[CG]C[CG]CTGCA
CTAACTTGC[CG]CTTGGTCT[CG]CCT[CG]C[CG]C[CG][CG
]GCT[CG]CTG[CG]CTTTGGGTGG[CG]GGGGG[CG]GGGAGAG
C
SEQ ID NO: 5333
ACACCTGCAGGAACA[CG][CG]CACACTACACAC[CG]TTGCTC
T[CG]CCTCC[CG]GTATCACCTT[CG]AA[CG][CG]CTGGC[C
G]GCCCCCTCCTAGCCTGGTGCAGT[CG]GCTC[CG]CTTCTACT
CAGTCT[CG]GGTCCTCCTGGGCCAA[CG]CAGACCCTGGTGCCT
CCTTCCTTAGA[CG]AGCTAAGGTCAAGACTTCTGCTGCTG[CG]
GAGGAGCC[CG][CG]CACCAGGC[CG]GGGTGGACTG[CG]CCT
G[CG]CAT[CG]GGCC[CG]CCTGGTTCTTAGGAACTA[CG]ACT
CCCAGGGGG[CG]CTG[CG][CG]CT[[CG]]C[CG]GCAC[CG]
[CG]ACTAAAGCTTGCAAAGCCTGGGTCAT[[CG]]AAACTACAA
CTCCCAGAAGGCTAAG[CG]TCTGCACA[CG]CTAGGTC[CG]CA
GGG[CG]CTGACCCCTGGGGGCC[CG]AGGAG[CG]C[CG]GGCT
GCTGCCC[CG]AGCCC[CG]CCCACTACAGCCTCAGTGCATGG[C
G]GTGGG[CG]CTTCCAGTAACTCAGGGGTGGGACTTTTTTCTTG
CTCAGGGGATGAGGC[CG]AGCCCACAGGCTTAGAAAGAAGGAAC
TTGAGTC[CG]GGCACCTGTCATCCCAG[CG]CTTAGGGAGGC[C
G]AGG[CG]GGTGGATCACCTGA
SEQ ID NO: 5334
GAAGGGGTGTGGAGGAGCCAGATGGGAAGAGAAGCAG[CG]GGA[
CG]AGGG[CG]AGGAAGGGCTGGAGATTGTTAGAGGACAGATGGG
G[CG]GGACTGGGGAGAGGGG[[CG]]AAGG[CG]A[CG][CG]C
[CG]AGGCACTCCTC[CG]CCCCATGAGCACAGACAGTAACTC[C
G]AAA[CG][CG]CAGATGGGC[CG]TAAGGTAAAGAGGCCTTTT
TTCCCTCTCCTCTC[CG]TCCTCTACCCTGCTCCCCTCCTAGACA
CTTGGTGAAAAAGCAAGGTG[CG]GTGTACCTGGAG[CG]AACAC
CATGATTTCTTCCAGC[CG]CTA[CG]CCCCCAGATTAAGAGAGA
AAGACAGGCAGA[CG]GAGGATGGGAAGAAGGC[CG]GACTTGG[
CG]GGGTC[CG]GTGCAGGAGGG[CG][CG]CTGGG[CG]GG[CG
]G[CG]GG[CG]GGG[CG]GTCAGCACCC[CG]GACAGCTCC[CG
]C[CG]CTAGATCCTGGGGC[CG]CAGCTCCAGC[CG]C[CG]C[
CG][CG][CG]CTCTGCCTCCACAATG[CG]GCCACAG[CG]G[C
G]G[CG]GGAGGAGGAGGGTGAATCCC[CG]CTCAGTCCATCCCT
CATTCCTGTGCCAGCT[CG]G[CG]A[CG]TACACTGCTGCTTAG
GGAGCAAAGCACTTCCAC[CG]C
SEQ ID NO: 5335
GCT[CG]GAAGGGGTGTGGAGGAGCCAGATGGGAAGAGAAGCAG[
CG]GGA[CG]AGGG[CG]AGGAAGGGCTGGAGATTGTTAGAGGAC
AGATGGGG[CG]GGACTGGGGAGAGGGG[[CG]]AAGG[CG]A[C
G][CG]C[CG]AGGCACTCCTC[CG]CCCCATGAGCACAGACAGT
AACTC[CG]AAA[CG][CG]CAGATGGGC[CG]TAAGGTAAAGAG
GCCTTTTTTCCCTCTCCTCTC[CG]TCCTCTACCCTGCTCCCCTC
CTAGACACTTGGTGAAAAAGCAAGGTG[CG]GTGTACCTGGAG[C
G]AACACCATGATTTCTTCCAGC[[CG]]CTA[CG]CCCCCAGAT
TAAGAGAGAAAGACAGGCAGA[CG]GAGGATGGGAAGAAGGC[CG
]GACTTGG[CG]GGGTC[CG]GTGCAGGAGGG[CG][CG]CTGGG
[CG]GG[CG]G[CG]GG[CG]GGG[CG]GTCAGCACCC[CG]GAC
AGCTCC[CG]C[CG]CTAGATCCTGGGGC[CG]CAGCTCCAGC[C
G]C[CG]C[CG][CG][CG]CTCTGCCTCCACAATG[CG]GCCAC
AG[CG]G[CG]G[CG]GGAGGAGGAGGGTGAATCCC[CG]CTCAG
TCCATCCCTCATTCCTGTGCCAGCT[[CG]]G[CG]A[[CG]]TA
CACTGCTGCTTAGGGAGCAAAGCACTTCC
SEQ ID NO: 5336
CCTT[CG]AA[CG][CG]CTGGC[CG]GCCCCCTCCTAGCCTGGT
GCAGT[CG]GCTC[CG]CTTCTACTCAGTCT[CG]GGTCCTCCTG
GGCCAA[CG]CAGACCCTGGTGCCTCCTTCCTTAGA[CG]AGCTA
AGGTCAAGACTTCTGCTGCTG[CG]GAGGAGCC[CG][CG]CACC
AGGC[CG]GGGTGGACTG[CG]CCTG[CG]CAT[CG]GGCC[CG]
CCTGGTTCTTAGGAACTA[CG]ACTCCCAGGGGG[CG]CTG[CG]
[CG]CT[CG]C[CG]GCAC[CG][CG]ACTAAAGCTTGCAAAGCC
TGGGTCAT[CG]AAACTACAACTCCCAGAAGGCTAAG[[CG]]TC
TGCACA[CG]CTAGGTC[CG]CAGGG[CG]CTGACCCCTGGGGGC
C[CG]AGGAG[CG]C[CG]GGCTGCTGCCC[CG]AGCCC[[CG]]
CCCACTACAGCCTCAGTGCATGG[CG]GTGGG[CG]CTTCCAGTA
ACTCAGGGGGGGACTTTTTTCTTGCTCAGGGGATGAGGC[CG]AG
CCCACAGGCTTAGAAAGAAGGAACTTGAGTC[CG]GGCACCTGTC
ATCCCAG[CG]CTTAGGGAGGC[CG]AGG[CG]GGTGGATCACCT
GAGGTCAGGAGTT[CG]AGACCAGCCTGGCCAATATGG[CG]AAA
CCCC[CG]TCTCTACTAA
SEQ ID NO: 5337
GACAAGCCACCTTCTCCATGCTCCTCTCCCCAAAGGGTAAGATTT
ATTCTAAGGGTTCTGAATT[CG]TCACATTTATAAACCC[CG]TG
GGTC[[CG]]GCACAGCAATTAACATTGGTCCAAA[CG]GTGTCA
GTGAACTTTAGCTGCAAAGAAACTACCTCATTTAAAATTTAAGCA
GACCACAGGGAGTAGCATTGGAGGAAAGAGAGGAGATTTTCTCCT
TCAACATTTTGAGCATACTGGAATGCCAACCCCCTAGGTAGGTGC
CAGGGCCACCACAGGAACCTGACT[CG]CAGGAGT[CG]TCCTC[
CG]TCTCTA[CG][CG]AAG[CG]CCCCCT[CG]AGGTGGGCAGG
GGGTGG[CG]G[CG]G[CG]A[CG]GTGG[CG]GAGC[CG]CAGA
G[CG]AGCTAGAGAG[CG]AGAG[CG]GTGAGACTCTG[CG]GA[
[CG]]TCTTCC[CG]CC[CG]C[CG][CG]CTC[CG]CCCCACCC
AGCCTC[CG]CAGCCT[CG]GGC[CG]GAT[CG]CC[CG]G[CG]
GGGAAGATG[CG]CCTCAAGAACCAGGTAGAAG[CG]CCT[CG]G
[[CG]]CAGGCCC[CG]CACCCC[CG]ACTCTGCC[CG]CCCTCT
GTTA[CG]GACACC[CG]CTGGGCCA[[CG]]TGGT[CG][CG]A
CTGGCTTCTCCCAG[CG]GCCAGCC
SEQ ID NO: 5338
CAC[CG]TTGCTCT[CG]CCTCC[CG]GTATCACCTT[CG]AA[C
G][CG]CTGGC[CG]GCCCCCTCCTAGCCTGGTGCAGT[CG]GCT
C[CG]CTTCTACTCAGTCT[CG]GGTCCTCCTGGGCCAA[CG]CA
GACCCTGGTGCCTCCTTCCTTAGA[CG]AGCTAAGGTCAAGACTT
CTGCTGCTG[CG]GAGGAGCC[CG][CG]CACCAGGC[CG]GGGT
GGACTG[CG]CCTG[CG]CAT[CG]GGCC[CG]CCTGGTTCTTAG
GAACTA[CG]ACTCCCAGGGGG[CG]CTG[CG][CG]CT[CG]C[
[CG]]GCAC[CG][CG]ACTAAAGCTTGCAAAGCCTGGGTCAT[C
G]AAACTACAACTCCCAGAAGGCTAAG[[CG]]TCTGCACA[CG]
CTAGGTC[CG]CAGGG[CG]CTGACCCCTGGGGGCC[CG]AGGAG
[CG]C[CG]GGCTGCTGCCC[CG]AGCCC[CG]CCCACTACAGCC
TCAGTGCATGG[CG]GTGGG[CG]CTTCCAGTAACTCAGGGGGGG
ACTTTTTTCTTGCTCAGGGGATGAGGC[CG]AGCCCACAGGCTTA
GAAAGAAGGAACTTGAGTC[CG]GGCACCTGTCATCCCAG[CG]C
TTAGGGAGGC[CG]AGG[CG]GGTGGATCACCTGAGGTCAGGAGT
T[CG]AGACCAGCCTGGCCA
SEQ ID NO: 5339
TGCCAAAGACAAGCCACCTTCTCCATGCTCCTCTCCCCAAAGGGT
AAGATTTATTCTAAGGGTTCTGAATT[CG]TCACATTTATAAACC
C[[CG]]TGGGTC[[CG]]GCACAGCAATTAACATTGGTCCAAA[
[CG]]GTGTCAGTGAACTTTAGCTGCAAAGAAACTACCTCATTTA
AAATTTAAGCAGACCACAGGGAGTAGCATTGGAGGAAAGAGAGGA
GATTTTCTCCTTCAACATTTTGAGCATACTGGAATGCCAACCCCC
TAGGTAGGTGCCAGGGCCACCACAGGAACCTGACT[[CG]]CAGG
AGT[CG]TCCTC[CG]TCTCTA[CG][CG]AAG[CG]CCCCCT[C
G]AGGTGGGCAGGGGGTGG[CG]G[CG]G[CG]A[CG]GTGG[CG
]GAGC[CG]CAGAG[CG]AGCTAGAGAG[CG]AGAG[[CG]]GTG
AGACTCTG[CG]GA[[CG]]TCTTCC[CG]CC[CG]C[CG][CG]
CTC[CG]CCCCACCCAGCCTC[CG]CAGCCT[CG]GGC[CG]GAT
[CG]CC[CG]G[CG]GGGAAGATG[CG]CCTCAAGAACCAGGTAG
AAG[CG]CCT[CG]G[[CG]]CAGGCCC[CG]CACCCC[CG]ACT
CTGCC[CG]CCCTCTGTTA[CG]GACACC[CG]CTGGGCCA[CG]
TGGT[CG][CG]ACTGGCTTCTCCCAG
SEQ ID NO: 5340
TACATTCCCTGCCAAAGACAAGCCACCTTCTCCATGCTCCTCTCC
CCAAAGGGTAAGATTTATTCTAAGGGTTCTGAATT[CG]TCACAT
TTATAAACCC[[CG]]TGGGTC[CG]GCACAGCAATTAACATTGG
TCCAAA[CG]GTGTCAGTGAACTTTAGCTGCAAAGAAACTACCTC
ATTTAAAATTTAAGCAGACCACAGGGAGTAGCATTGGAGGAAAGA
GAGGAGATTTTCTCCTTCAACATTTTGAGCATACTGGAATGCCAA
CCCCCTAGGTAGGTGCCAGGGCCACCACAGGAACCTGACT[CG]C
AGGAGT[CG]TCCTC[[CG]]TCTCTA[CG][CG]AAG[CG]CCC
CCT[CG]AGGTGGGCAGGGGGTGG[CG]G[CG]G[CG]A[CG]GT
GG[CG]GAGC[CG]CAGAG[CG]AGCTAGAGAG[CG]AGAG[[CG
]]GTGAGACTCTG[CG]GA[[CG]]TCTTCC[CG]CC[CG]C[CG
][CG]CTC[CG]CCCCACCCAGCCTC[CG]CAGCCT[CG]GGC[C
G]GAT[CG]CC[CG]G[CG]GGGAAGATG[CG]CCTCAAGAACCA
GGTAGAAG[CG]CCT[CG]G[CG]CAGGCCC[CG]CACCCC[CG]
ACTCTGCC[CG]CCCTCTGTTA[CG]GACACC[CG]CTGGGCCA[
[CG]]TGGT[CG][CG]ACTGGCTT
SEQ ID NO: 5341
CA[CG][CG]GTCACACACCTGCAGGAACA[CG][CG]CACACTA
CACAC[CG]TTGCTCT[CG]CCTCC[CG]GTATCACCTT[CG]AA
[CG][CG]CTGGC[CG]GCCCCCTCCTAGCCTGGTGCAGT[CG]G
CTC[CG]CTTCTACTCAGTCT[CG]GGTCCTCCTGGGCCAA[CG]
CAGACCCTGGTGCCTCCTTCCTTAGA[CG]AGCTAAGGTCAAGAC
TTCTGCTGCTG[CG]GAGGAGCC[CG][[CG]]CACCAGGC[CG]
GGGTGGACTG[CG]CCTG[CG]CAT[CG]GGCC[CG]CCTGGTTC
TTAGGAACTA[CG]ACTCCCAGGGGG[CG]CTG[CG][CG]CT[[
CG]]C[CG]GCAC[CG][[CG]]ACTAAAGCTTGCAAAGCCTGGG
TCAT[[CG]]AAACTACAACTCCCAGAAGGCTAAG[CG]TCTGCA
CA[CG]CTAGGTC[CG]CAGGG[CG]CTGACCCCTGGGGGCC[CG
]AGGAG[CG]C[CG]GGCTGCTGCCC[CG]AGCCC[CG]CCCACT
ACAGCCTCAGTGCATGG[CG]GTGGG[CG]CTTCCAGTAACTCAG
GGGTGGGACTTTTTTCTTGCTCAGGGGATGAGGC[CG]AGCCCAC
AGGCTTAGAAAGAAGGAACTTGAGTC[CG]GGCACCTGTCATCCC
AG[CG]CTTAGGGAGGC[CG]AGG[CG]GGT
SEQ ID NO: 5342
GTTCCTTCT[CG]CCCAGCCTTGCAATCCATGC[CG]AGAGGAAG
GCAGTG[CG]AGCC[CG][CG]CCAG[CG]CCCAGCTCC[[CG]]
GGACAGGGT[CG]GCAATGCTGCTGAGCAGAACTTGAT[CG][CG
]CTCCTTCCT[CG]CTGCTAGTGGAAG[CG]ATGCTGCA[CG]GC
ACAGCTAG[[CG]]CTTCCC[CG]GCTCTCCTTCAAGCTGAAGGT
TAC[CG]ACC[CG][CG]CAGCCAGCCCCAGCACTGTGAGCTG[C
G][CG]CCTCAGGTC[CG]GGCTC[CG]GCTGCTTGG[CG]G[CG
]G[CG]CCCAGGGCAACAAC[CG]GGC[CG]CC[CG][CG]C[CG
]GGG[CG]CACTGCACCAG[CG]GCTT[CG]GCTTGGTGGATGTG
TATGCATGAGTTCTGCAC[CG]AATGGG[CG]CAAAAAG[CG]CC
CCAGC[[CG]]GTCCACC[CG]CTCCT[CG]ATCTTCCAGATCAG
CAAGCCCC[CG]CTGCAGAG[CG]GAGATTGGGAG[CG]CAGGGG
CAG[CG]GCTC[CG]AGAG[CG]CCCACAAAACCCAA[[CG]]AG
CCCTGGA[CG]ACTGCAAGATGGTGGGTGAAAACTG[CG]CCTCT
TTTTTTTTTTTTTTAATTGAGAGGGGGTGGGGGGAGTCATGTATG
TTAATCATTTGCCTGAGTGCCAGCCACTC
SEQ ID NO: 5343
GGG[CG]GAGGCAAGGACTCTAGCTTGAGGTTCTTTGAGGCAGAG
CAGAAATTAGCCCCTAGGTGTGC[[CG]]TC[CG]TGG[CG][CG
]AGTCA[CG]GAACTCCAGGTACCC[CG]GGGT[CG]TGTC[CG]
CACCTTTGTCCTCAGTCCCCAGAAG[CG]T[CG]AAATTTCTTAG
CA[CG]ACACCCCTGCCT[CG][CG]CTACATCTCTTCCCCCTAG
GGGGTTGGTGGCCTC[CG]TGCTCAAAGGAGGCAG[CG]GGAA[C
G]CCTAACTTCTAAGATGCCCTTAATTGATCTCCTCCTTCTTAAC
TACAG[CG]AGGGAATAACAACT[[CG]]GGGCTGGGGACTGGGT
ATCTAAGACT[CG]CTCTC[CG]CAGGGTC[CG]C[CG]GCAGGG
[CG]GCCACAGGACC[CG]GGACTGGAACTGGCC[CG]GGAGGAC
AC[CG]G[CG][CG]TGTCAGG[CG]CAGAG[CG]TGAAACAGGC
AAGCTG[CG][CG]CAGAGCAGGGCTTGGATGG[CG]GATCTTGG
GCACAATGAGCCC[CG]GATCCTGAAGGAAGATAACCCCTGCTGA
GACCCTGTC[CG]GAGCCTTGGAGCTTTTCAGCAGTTTAGGCT[C
G]GATCTC[CG]GCCTGGGTTTT[CG]GTGCAGAGCTGCTTGAAG
AGTTG
SEQ ID NO: 5344
GCAGC[CG]G[CG]GTTTGTGCCCATCTGTGGGGGCAAGTCCTAC
CTTCTGCCAAAAGG[CG][CG]A[CG]CATGCACAAAAGATGGAT
CCAGGCTATCTTTCTCTGCCATCAGCTCAGGCAAATATTTCTCCT
CTTCCATAG[CG][CG]GACTT[CG]GATTGTCCC[[CG]]GG[C
G]AAG[CG][CG]AGGTTC[CG]CT[CG]CT[CG]GA[CG]CAGG
CAGGGTCTTGGGGCAG[CG]CCTGGCTCC[CG][CG]CTGCTCCT
CCTC[CG][CG][CG]G[[CG]]AGGGATCTCTGTG[CG]TCCTC
ACTGGCCCATGCACCCAGCACCTG[CG]ACTCC[CG]C[CG]T[C
G]GGCTG[CG]TGGCCC[CG][CG]CCCACACCTGCC[CG]TCCC
TTC[CG]T[CG]TCCCT[CG]CT[CG][CG]CAGAGCCC[CG]GC
TCACACCAG[CG]GCCTTAACTGGAGAGG[[CG]]GGAACAGGA[
[CG]][CG]GCCCACCTC[CG]CCAACCACTCATTGGTGGTGGTG
CCAGGAGAGG[CG]GGG[[CG]]CTGGCAGAAGGC[CG][CG]GG
CTCTCATTGGG[[CG]]GTAG[CG]CAAGT[CG]GGTGGCCTGAC
TGGCAGGAGGCAGCTGCAGAGGGGAAGGGAGCT[CG]GAAGATGC
AAA[CG]CTTTCTTCTTG[CG]CCCAAGCCTTCCCTGGCTATT
SEQ ID NO: 5345
AGAGGGCCCAC[CG]AGCACTA[CG]G[CG]GGTG[CG]CA[CG]
CCC[CG]GGG[CG]CT[CG]GCAGGA[CG]ACAGTCTGCACAGCC
[CG]AAGG[[CG]]GAAA[CG]AGCATCAACTGCACAAAGTCCTG
GGGTCCTGGAGCATCCCCTC[CG][CG]TCCTTCCTCCCTCTGGG
GCTGGGGACAGC[CG]GGATGTCCCAGGCTGAGGTGGCCACCAGC
[CG]AG[CG][CG]GCTGCTAGGA[CG]CTGG[CG]TGGGGAG[C
G][CG]G[CG][CG]GAACTA[CG]GACAGTGAGCCCTGG[CG]C
T[CG]CTGCCCTG[CG]CCTTAATTTGCTGG[CG]G[CG]G[CG]
ATCC[[CG]]GAGGCC[CG]CAGCCAGTCAG[CG]C[CG]TCTCA
[CG]TCAC[CG]CTTCCTGATTC[CG]C[CG]C[CG]GGGG[CG]
GGGC[CG][CG]GGC[CG]GG[CG][CG]GAGGG[CG][CG]CCC
AGGGTG[CG]G[CG]CC[CG][[CG]]TGGCCTGT[CG]CCC[CG
]GCTGTT[[CG]]GTACCCCAGCACAGGTTCAGGGAAAAGGGTGC
CACCACTAGGCTGA[CG]CAGCAGCCATGGACATCCCCACCTGGT
CTCACAGCCC[CG]GG[CG]GGAGGG[CG]GTG[CG][CG]CTGT
[CG]GGAGAGGACACATCAC[CG][CG]GCCTAA[CG]CC[CG]A
GATC[CG]GGCATGGCCCAGTT
SEQ ID NO: 5346
AG[CG]GC[CG][CG]GGCAGCCC[CG]GG[CG]G[CG]G[CG]G
GGG[CG]G[CG]GCAGGGGGGG[CG]G[CG]G[CG]G[CG]GCTG
TAG[CG]GACACTGGGGCTGGGGCTGGGG[CG]AGGGTGG[CG]G
[CG]G[CG]GGGGCAGGTGGTGATGGGGATGCAGGAGG[CG]GAA
CTGGGGCTGC[CG]GAG[CG]GGTACTGGTGGTGCTGCT[CG]TG
CCTCCAGAGATGCTTGGGGGCTTTGAG[[CG]]TGGCCC[CG]C[
CG]G[CG]CTGT[CG]CTGCCCTCTC[CG]CTGCC[CG]CC[CG]
GGC[CG]G[CG][CG]CTGCTGCCCTCTGCCTCCATCCTGGCT[C
G][CG]GCTC[CG][[CG]]ACCTGCTGCCCAGCC[CG]GGTTCA
C[CG][CG]CTGGCC[CG]AG[CG]CCTTCCTGATGCTGCTGCTG
CTGCTGC[CG]C[CG]C[CG]C[CG]CTTCTGCAGCCCAGCAGAG
GCTGGTGCTTAGCTTCTCACTTGAAGG[CG]CCAGAGCCTCAGGG
CTAC[CG]AGAGGGGGCCCTGCCAG[CG]GGAGGGGTCACAGAA[
CG][CG]GCAGGGCTCCTTCCAC[CG]AGGCTATGGCAGAGACA[
CG]CTCC[CG]GGCTCCCCAAGTCACCAAGACTAGGGTG[[CG]]
[CG]GTGCC[CG]GTTT[CG]CCAGAGGTTGGTCCTTCTTCCTCC
CT
SEQ ID NO: 5347
GC[CG][CG]A[CG]GGACCACAGAAAT[CG]ACCAG[CG]GCCT
GTTTAAGGAG[CG]ACAAGGCCCAGCCCTCTCCTGCATTGTGG[C
G]A[CG]CCATTTCCCTG[CG]CCCCCAG[CG][CG]GC[CG]CT
TGGCTTTTGTTTGAGGTGA[CG]CTGGG[CG]GCAGCATCCTTCC
CTCAGCCCTGGGGACCAG[CG]GGGACTACAGAACCCAGAAGGTT
TTGTCTCCAGC[[CG]]CAGGGATGCTGAGCCAATC[[CG]][CG
]CTGAGAAAAGGGT[CG]TTTC[[CG]]CTTTGGGACCAATGGGT
[CG]GGGAGGGACTTC[CG]GTATCACTTCAGTGG[CG]GTCATT
TTTGCAG[CG]CTTGGGTGCATCCAGAC[CG]TCAGAGCTTTGGG
AG[CG]CTTTGTTTGG[[CG]]ACAGT[[CG]]GAAGG[[CG]][
CG]AGGGGAGGGGTCCTCC[CG]CTGAACAGTGGGGGTTCTAAGG
GT[CG]G[CG]G[CG]G[CG]GGGTTGA[CG]GCTTTGCCTAGGT
CCCTC[CG]CC[CG]TAGCTGT[CG]GGTCC[CG]GCCC[CG]CT
CTGCCCACAGACTC[CG]ATGGCTG[CG]GC[CG][CG]CTGAGG
GCCC[CG]ACTCAGGTGAG[CG]CTGCCTCTACTGGGCCTCACCC
TCCATCCCCAAATTAGTGCCTTCTTGGGTCA
SEQ ID NO: 5348
GGGACCAATCAC[[CG]]GCTCTGAGAAAGTT[CG]GCAGTGGCC
ACCACATCTGGTTCT[CG]TTAACTTTTCTAAGGCAG[CG]GC[[
CG]]CTGGAGCAG[[CG]]GGGCTGG[CG]GGGTAAAAGCTCCTG
GCCAGGGCTGCCTGGAGCTGCCCCTTCCACTC[CG]CCCCCAGGG
AGCTCC[CG]GGTCATCCTCTCATC[CG]GGCTGCCC[[CG]][C
G]GCCCCCAAGGAGCCCCACCCC[CG]GGACCAAATGGCC[CG]C
AAGGTTTGGGGCAG[CG]G[CG]TTGCAGGAGATGAGCTCAG[[C
G]]CAAAGGGAACCC[CG]CAG[CG]G[CG]AGTG[[CG]]GCTG
CTGGCCTG[CG][CG]CTGTGGCCCCAACAGGCTGGCAGGG[CG]
[CG]GG[CG]GGTGG[CG]GGGTTG[CG]GTATGAGCTTTGCTCC
CTGCCCTGGGGTCC[CG]GG[CG]CTCCTGGCTCTGGAGGCCTGG
CCAT[CG]GTC[CG]AAAAGCAAAGGGACATTGT[CG]TGGTCCC
CT[CG]GC[CG]CTT[CG]CTCCACCCAGAGA[CG]CCCTGAGGA
GAAG[CG]AGCCTTTGTA[CG]AAGGCAGAGGGCTGACTCCTAAG
TTCTGTCCCCCTCC[CG]AAGGC[CG]GC[CG]CTAGGGAAGACT
TGGCCACCCTCTGGAAGTACC
SEQ ID NO: 5349
CAAGCTAATTGGCTGAGGCCCT[CG]GCTGGGGACCAATCAC[CG
]GCTCTGAGAAAGTT[CG]GCAGTGGCCACCACATCTGGTTCT[C
G]TTAACTTTTCTAAGGCAG[CG]GC[[CG]]CTGGAGCAG[[CG
]]GGGCTGG[CG]GGGTAAAAGCTCCTGGCCAGGGCTGCCTGGAG
CTGCCCCTTCCACTC[[CG]]CCCCCAGGGAGCTCC[CG]GGTCA
TCCTCTCATC[CG]GGCTGCCC[[CG]][CG]GCCCCCAAGGAGC
CCCACCCC[CG]GGACCAAATGGCC[CG]CAAGGTTTGGGGCAG[
CG]G[CG]TTGCAGGAGATGAGCTCAG[[CG]]CAAAGGGAACCC
[CG]CAG[CG]G[CG]AGTG[[CG]]GCTGCTGGCCTG[CG][CG
]CTGTGGCCCCAACAGGCTGGCAGGG[CG][CG]GG[CG]GGTGG
[CG]GGGTTG[CG]GTATGAGCTTTGCTCCCTGCCCTGGGGTCC[
CG]GG[CG]CTCCTGGCTCTGGAGGCCTGGCCAT[CG]GTC[CG]
AAAAGCAAAGGGACATTGT[CG]TGGTCCCCT[CG]GC[CG]CTT
[CG]CTCCACCCAGAGA[CG]CCCTGAGGAGAAG[CG]AGCCTTT
GTA[CG]AAGGCAGAGGGCTGACTCCTAAGTTCTGTCCCCCTCC[
CG]AAGGC[CG]GC[CG]CTAGG
SEQ ID NO: 5350
TGGGCCAA[CG]CAGACCCTGGTGCCTCCTTCCTTAGA[CG]AGC
TAAGGTCAAGACTTCTGCTGCTG[CG]GAGGAGCC[CG][[CG]]
CACCAGGC[CG]GGGTGGACTG[CG]CCTG[CG]CAT[CG]GGCC
[CG]CCTGGTTCTTAGGAACTA[CG]ACTCCCAGGGGG[CG]CTG
[CG][CG]CT[[CG]]C[[CG]]GCAC[CG][CG]ACTAAAGCTT
GCAAAGCCTGGGTCAT[[CG]]AAACTACAACTCCCAGAAGGCTA
AG[[CG]]TCTGCACA[CG]CTAGGTC[CG]CAGGG[CG]CTGAC
CCCTGGGGGCC[CG]AGGAG[CG]C[CG]GGCTGCTGCCC[CG]A
GCCC[CG]CCCACTACAGCCTCAGTGCATGG[CG]GTGGG[CG]C
TTCCAGTAACTCAGGGGGGGACTTTTTTCTTGCTCAGGGGATGAG
GC[CG]AGCCCACAGGCTTAGAAAGAAGGAACTTGAGTC[CG]GG
CACCTGTCATCCCAG[CG]CTTAGGGAGGC[CG]AGG[CG]GGTG
GATCACCTGAGGTCAGGAGTT[CG]AGACCAGCCTGGCCAATATG
G[CG]AAACCCC[CG]TCTCTACTAAAAATACAAAAATTAGC[CG
]AGCATGGTGGCATGCACTTCCAGCTACTTGGAAGGCTGAGG[CG
]GGAGAATGGCTTG
SEQ ID NO: 5351
TGGTTCCCTTCCA[CG]GGGGTCC[CG]AGGTGCTTTGAGGAGGG
AGAAAGAGGG[CG]TCCCCTCTGGGGAGCCCACTCTC[CG]GGCT
TCTACTGACCTGGTCTC[CG]CCTCAC[CG]GCCTCTTG[CG]GC
[CG]CTGCAGAAG[CG]CACTTTGCTGAACACCC[CG]AGGA[CG
]TGCCTCT[CG]CACAGGGAG[CG]CC[CG]TCTTTGCTGGGGCT
GGAG[CG]G[CG]CTTGGAGGC[CG]ACACT[CG]GT[CG]CTGT
TGGACTCCCT[CG]CCTGC[CG]CTTCTGC[CG]GATCAAGGAGC
TGGCTAT[CG]C[CG]CAGCCATAGCTGCTCAG[CG]AGGGCCTC
AGGCCCCAGCCTCTACTG[CG]CCCTC[CG]GCTTG[CG]CTC[C
G]C[CG]GGG[CG]AGGGCAGGACCTGGG[CG]GCCAGGGAAAGG
GCAGT[CG][CG]GGGAGGCAGTGCTAAAATTTGAGGAGGCTGCA
GTAT[CG]AAAACC[CG]G[[CG]]CTCACAAGGTTAGTCAAAGT
CTGGGCAGTGG[CG]ACAAAATGTGTGAAAATCCAGATGTAAACT
TCCCCAACCTCTGG[CG]GC[CG]GGGGG[CG]GGG[CG]GGG[C
G]GTCCCAGGCCCTCTTG[CG]AAGTAGA[CG]TTTGCACCCCAA
ACTTGCACCCCAAGG
SEQ ID NO: 5352
TAAGCAGGTGCAGATGCAGTGACAGGCTCACAGGCAGTCACACAC
CTGCACCTGCTGGTAC[CG]T[CG]AGTGACCAGGTGCAGA[CG]
CTGTGTTACTCAGGGA[CG]GGCTCACAGGCAGTCACACACCTAT
A[[CG]]GGCTGACAC[CG]CTAAGTGAACAGGTGCAGACACAAT
GGCACT[CG]GGGA[CG]GGCTCACA[CG][CG]GTCACACACCT
GCAGGAACA[CG][CG]CACACTACACAC[CG]TTGCTCT[CG]C
CTCC[CG]GTATCACCTT[CG]AA[CG][CG]CTGGC[CG]GCCC
CCTCCTAGCCTGGTGCAGT[CG]GCTC[[CG]]CTTCTACTCAGT
CT[CG]GGTCCTCCTGGGCCAA[CG]CAGACCCTGGTGCCTCCTT
CCTTAGA[CG]AGCTAAGGTCAAGACTTCTGCTGCTG[CG]GAGG
AGCC[CG][[CG]]CACCAGGC[CG]GGGTGGACTG[CG]CCTG[
CG]CAT[CG]GGCC[CG]CCTGGTTCTTAGGAACTA[CG]ACTCC
CAGGGGG[CG]CTG[CG][CG]CT[CG]C[CG]GCAC[CG][[CG
]]ACTAAAGCTTGCAAAGCCTGGGTCAT[CG]AAACTACAACTCC
CAGAAGGCTAAG[CG]TCTGCACA[CG]CTAGGTC[CG]CAGGG[
CG]CTGACCCCTGGGGGCC[CG]AG
SEQ ID NO: 5353
GAGAGGAGAGGGCCCAC[CG]AGCACTA[CG]G[CG]GGTG[CG]
CA[CG]CCC[CG]GGG[CG]CT[CG]GCAGGA[CG]ACAGTCTGC
ACAGCC[CG]AAGG[[CG]]GAAA[CG]AGCATCAACTGCACAAA
GTCCTGGGGTCCTGGAGCATCCCCTC[CG][CG]TCCTTCCTCCC
TCTGGGGCTGGGGACAGC[CG]GGATGTCCCAGGCTGAGGTGGCC
ACCAGC[CG]AG[CG][CG]GCTGCTAGGA[CG]CTGG[CG]TGG
GGAG[CG][CG]G[CG][CG]GAACTA[CG]GACAGTGAGCCCTG
G[CG]CT[CG]CTGCCCTG[CG]CCTTAATTTGCTGG[CG]G[CG
]G[CG]ATCC[CG]GAGGCC[CG]CAGCCAGTCAG[CG]C[CG]T
CTCA[CG]TCAC[CG]CTTCCTGATTC[CG]C[CG]C[CG]GGGG
[CG]GGGC[CG][CG]GGC[CG]GG[CG][CG]GAGGG[CG][CG
]CCCAGGGTG[CG]G[CG]CC[CG][[CG]]TGGCCTGT[CG]CC
C[CG]GCTGTT[CG]GTACCCCAGCACAGGTTCAGGGAAAAGGGT
GCCACCACTAGGCTGA[CG]CAGCAGCCATGGACATCCCCACCTG
GTCTCACAGCCC[CG]GG[CG]GGAGGG[CG]GTG[CG][CG]CT
GT[CG]GGAGAGGACACATCAC[CG][CG]GCCTAA[CG]CC[CG
]AGATC[CG]GGCATGGC
SEQ ID NO: 5354
GCAGCAGCAGTAGCACTGGTAA[CG]A[CG]ACAGCAC[CG]GC[
CG]CC[CG]GCCACC[CG]GTCCCCCTCCATGCT[CG]CT[CG]G
C[CG]TG[CG]CTTACC[CG]CTGCACAA[CG][CG]TCC[CG]C
CC[CG]GCCTCCAGCCCACTCTC[CG][CG]C[CG][CG]CCAGC
CT[CG]AACCTGGATCTC[CG][CG]GG[CG]CCTGGG[CG]GGG
[CG]GGACTCCAGGCCCACCCAGCAAGCTAATTGGCTGAGGCCCT
[CG]GCTGGGGACCAATCAC[[CG]]GCTCTGAGAAAGTT[CG]G
CAGTGGCCACCACATCTGGTTCT[CG]TTAACTTTTCTAAGGCAG
[CG]GC[[CG]]CTGGAGCAG[CG]GGGCTGG[CG]GGGTAAAAG
CTCCTGGCCAGGGCTGCCTGGAGCTGCCCCTTCCACTC[[CG]]C
CCCCAGGGAGCTCC[CG]GGTCATCCTCTCATC[CG]GGCTGCCC
[[CG]][CG]GCCCCCAAGGAGCCCCACCCC[CG]GGACCAAATG
GCC[CG]CAAGGTTTGGGGCAG[CG]G[CG]TTGCAGGAGATGAG
CTCAG[CG]CAAAGGGAACCC[CG]CAG[CG]G[CG]AGTG[CG]
GCTGCTGGCCTG[CG][CG]CTGTGGCCCCAACAGGCTGGCAGGG
[CG][CG]GG[CG]GGTGG[CG]GGGTTG[CG]GTATGAGC
SEQ ID NO: 5355
AAGTC[CG]CCCCCCTCAC[CG]GGGCATGGT[CG]GAGGGTGGC
TGCTG[CG]CTC[CG]GGGCAGTTCTTCTCCTGCTTC[CG]GGGG
G[CG]GGGAAGGAGCAGACACAGCAGACACACAGAGAAAGAGAGG
CAGGGATTATTGCC[CG]AAAC[CG]CCAGC[CG]C[CG]GCAGC
CTC[CG]C[CG]ACCCTCCCTGCTCCCCAGGTC[CG][CG][CG]
CAGCCTGCAGCCTCTCAGC[CG]GGTTC[CG]GCAGTG[CG]TCC
AGGG[CG]C[CG]AGGGGAAGGGCTGGGCCC[CG]GGGACTGGGG
[CG][CG]GTGGGGGCACACAGACTCTCCAGGT[[CG]][CG][C
G][CG]CCCTTCCTGCTCCTCTGCTATTTTCCTGAACTCCCCAGA
ACCCCCAGTTGCCTGGCTTCCAT[CG]CC[CG]CAACTAG[CG]C
[CG]CTGT[CG]AGATTTTC[CG]CAATACAACTGCAGGGGT[[C
G]]TTATTTCCT[[CG]]CCTA[CG]GATCCCTA[CG]CTTCCCA
ACTCAGAGAGGAGGAGGACAGGTGGGGG[CG]GGAGGGGGAGGAG
GAGAAGGAGGCTTGGCCAGGTGAG[CG]TCCT[CG]C[CG]C[CG
]C[CG]GG[CG]TCCCAG[CG]TCAGCTC[CG]CAGCC[CG]GCA
CC[CG]GGGG[CG]GAGGCAGGAGGCC[CG]GG
SEQ ID NO: 5356
AGGCAAATATTTCTCCTCTTCCATAG[CG][CG]GACTT[CG]GA
TTGTCCC[[CG]]GG[CG]AAG[CG][CG]AGGTTC[CG]CT[CG
]CT[CG]GA[CG]CAGGCAGGGTCTTGGGGCAG[CG]CCTGGCTC
C[CG][CG]CTGCTCCTCCTC[CG][CG][CG]G[CG]AGGGATC
TCTGTG[CG]TCCTCACTGGCCCATGCACCCAGCACCTG[CG]AC
TCC[CG]C[[CG]][[CG]]GGCTG[CG]TGGCCC[CG][CG]CC
CACACCTGCC[CG]TCCCTTC[CG]T[CG]TCCCT[CG]CT[CG]
[CG]CAGAGCCC[CG]GCTCACACCAG[CG]GCCTTAACTGGAGA
GG[CG]GGAACAGGA[CG][CG]GCCCACCTC[CG]CCAACCACT
CATTGGTGGTGGTGCCAGGAGAGG[CG]GGG[CG]CTGGCAGAAG
GC[CG][CG]GGCTCTCATTGGG[[CG]]GTAG[CG]CAAGT[CG
]GGTGGCCTGACTGGCAGGAGGCAGCTGCAGAGGGGAAGGGAGCT
[CG]GAAGATGCAAA[CG]CTTTCTTCTTG[[CG]]CCCAAGCCT
TCCCTGGCTATTAGGCATGCCCAGTTTTGTGGAGTTCTCTGTGTC
TTCAGCTAAGG[CG]CAGAGGAGAGAGAG[CG][CG]GGAAAAG[
CG]CAGGTTAAAGAGAGAGGTCTGAGTACAAGTGGACA
SEQ ID NO: 5357
AGCATCCTTCCCTCAGCCCTGGGGACCAG[CG]GGGACTACAGAA
CCCAGAAGGTTTTGTCTCCAGC[CG]CAGGGATGCTGAGCCAATC
[[CG]][CG]CTGAGAAAAGGGT[CG]TTTC[CG]CTTTGGGACC
AATGGGT[CG]GGGAGGGACTTC[CG]GTATCACTTCAGTGG[CG
]GTCATTTTTGCAG[CG]CTTGGGTGCATCCAGAC[CG]TCAGAG
CTTTGGGAG[CG]CTTTGTTTGG[[CG]]ACAGT[CG]GAAGG[C
G][CG]AGGGGAGGGGTCCTCC[CG]CTGAACAGTGGGGGTTCTA
AGGGT[CG]G[CG]G[CG]G[CG]GGGTTGA[[CG]]GCTTTGCC
TAGGTCCCTC[CG]CC[CG]TAGCTGT[CG]GGTCC[CG]GCCC[
CG]CTCTGCCCACAGACTC[CG]ATGGCTG[CG]GC[CG][CG]C
TGAGGGCCC[CG]ACTCAGGTGAG[CG]CTGCCTCTACTGGGCCT
CACCCTCCATCCCCAAATTAGTGCCTTCTTGGGTCACTA[CG]GT
[CG]AGATCCTCATGTCCAGTACAGTGGGGGCT[CG]TGGGTGGG
GTCCCTATTTCCAGAACTTGGGTGATGGGGCA[CG]GAAGAGGGT
TACAGGCAAAGGGACCAG[CG]TTTCTAAACTCTTGGAGACACAG
TGAAGAA
SEQ ID NO: 5358
CCAGCA[CG][[CG]]GTCCTGGCCAGGGA[CG]GGGT[CG]TC[
CG]AACTGC[CG]TCCAGATTCCCCAAGGGAGACAAAGACC[CG]
AAACACAGCTCAAAGTTTC[CG]AGAGCAGTCACAG[CG]GGGCC
AGGGACTCCAGAAGTGTCAGCTCCAA[CG]ACTCCAGAGCTGCAC
ACTGGCCTCTATTCCCCAC[CG]CAAAGCCCCAGAGC[CG]CAGA
GACTT[CG]AAGGCAGC[CG]GAGAGGAGAGGGCCCAC[CG]AGC
ACTA[CG]G[CG]GGTG[CG]CA[CG]CCC[CG]GGG[CG]CT[C
G]GCAGGA[CG]ACAGTCTGCACAGCC[CG]AAGG[[CG]]GAAA
[CG]AGCATCAACTGCACAAAGTCCTGGGGTCCTGGAGCATCCCC
TC[CG][CG]TCCTTCCTCCCTCTGGGGCTGGGGACAGC[CG]GG
ATGTCCCAGGCTGAGGTGGCCACCAGC[CG]AG[CG][CG]GCTG
CTAGGA[CG]CTGG[CG]TGGGGAG[CG][CG]G[CG][CG]GAA
CTA[CG]GACAGTGAGCCCTGG[CG]CT[CG]CTGCCCTG[CG]C
CTTAATTTGCTGG[CG]G[CG]G[CG]ATCC[CG]GAGGCC[CG]
CAGCCAGTCAG[CG]C[CG]TCTCA[CG]TCAC[CG]CTTCCTGA
TTC[CG]C[CG]C[CG]GGGG[CG]GGGC[CG][CG]GGC[CG]G
SEQ ID NO: 5359
TCCTGCATTGTGG[CG]A[CG]CCATTTCCCTG[CG]CCCCCAG[
CG][CG]GC[CG]CTTGGCTTTTGTTTGAGGTGA[CG]CTGGG[C
G]GCAGCATCCTTCCCTCAGCCCTGGGGACCAG[CG]GGGACTAC
AGAACCCAGAAGGTTTTGTCTCCAGC[CG]CAGGGATGCTGAGCC
AATC[[CG]][CG]CTGAGAAAAGGGT[CG]TTTC[[CG]]CTTT
GGGACCAATGGGT[CG]GGGAGGGACTTC[CG]GTATCACTTCAG
TGG[[CG]]GTCATTTTTGCAG[CG]CTTGGGTGCATCCAGAC[C
G]TCAGAGCTTTGGGAG[CG]CTTTGTTTGG[[CG]]ACAGT[[C
G]]GAAGG[[CG]][CG]AGGGGAGGGGTCCTCC[CG]CTGAACA
GTGGGGGTTCTAAGGGT[CG]G[CG]G[CG]G[CG]GGGTTGA[[
CG]]GCTTTGCCTAGGTCCCTC[CG]CC[CG]TAGCTGT[CG]GG
TCC[CG]GCCC[CG]CTCTGCCCACAGACTC[CG]ATGGCTG[CG
]GC[CG][CG]CTGAGGGCCC[CG]ACTCAGGTGAG[CG]CTGCC
TCTACTGGGCCTCACCCTCCATCCCCAAATTAGTGCCTTCTTGGG
TCACTA[CG]GT[CG]AGATCCTCATGTCCAGTACAGTGGGGGCT
[CG]TGGGTGGGGTCCCTATTTCCAGA
SEQ ID NO: 5360
GCCATCAGCTCAGGCAAATATTTCTCCTCTTCCATAG[CG][CG]
GACTT[CG]GATTGTCCC[CG]GG[CG]AAG[CG][CG]AGGTTC
[CG]CT[CG]CT[CG]GA[CG]CAGGCAGGGTCTTGGGGCAG[CG
]CCTGGCTCC[CG][CG]CTGCTCCTCCTC[CG][CG][CG]G[C
G]AGGGATCTCTGTG[CG]TCCTCACTGGCCCATGCACCCAGCAC
CTG[CG]ACTCC[CG]C[CG]T[CG]GGCTG[CG]TGGCCC[CG]
[CG]CCCACACCTGCC[CG]TCCCTTC[CG]T[CG]TCCCT[CG]
CT[CG][CG]CAGAGCCC[CG]GCTCACACCAG[CG]GCCTTAAC
TGGAGAGG[[CG]]GGAACAGGA[CG][CG]GCCCACCTC[CG]C
CAACCACTCATTGGTGGTGGTGCCAGGAGAGG[CG]GGG[[CG]]
CTGGCAGAAGGC[CG][CG]GGCTCTCATTGGG[CG]GTAG[CG]
CAAGT[CG]GGTGGCCTGACTGGCAGGAGGCAGCTGCAGAGGGGA
AGGGAGCT[CG]GAAGATGCAAA[CG]CTTTCTTCTTG[[CG]]C
CCAAGCCTTCCCTGGCTATTAGGCATGCCCAGTTTTGTGGAGTTC
TCTGTGTCTTCAGCTAAGG[CG]CAGAGGAGAGAGAG[CG][CG]
GGAAAAG[CG]CAGGTTAAAGAGAGAGGTCTGAGT
SEQ ID NO: 5361
CAGCAGTAGCACTGGTAA[CG]A[CG]ACAGCAC[CG]GC[CG]C
C[CG]GCCACC[CG]GTCCCCCTCCATGCT[CG]CT[CG]GC[CG
]TG[CG]CTTACC[CG]CTGCACAA[CG][CG]TCC[CG]CCC[C
G]GCCTCCAGCCCACTCTC[CG][CG]C[CG][CG]CCAGCCT[C
G]AACCTGGATCTC[CG][CG]GG[CG]CCTGGG[CG]GGG[CG]
GGACTCCAGGCCCACCCAGCAAGCTAATTGGCTGAGGCCCT[CG]
GCTGGGGACCAATCAC[CG]GCTCTGAGAAAGTT[CG]GCAGTGG
CCACCACATCTGGTTCT[CG]TTAACTTTTCTAAGGCAG[[CG]]
GC[CG]CTGGAGCAG[[CG]]GGGCTGG[CG]GGGTAAAAGCTCC
TGGCCAGGGCTGCCTGGAGCTGCCCCTTCCACTC[CG]CCCCCAG
GGAGCTCC[CG]GGTCATCCTCTCATC[CG]GGCTGCCC[[CG]]
[CG]GCCCCCAAGGAGCCCCACCCC[CG]GGACCAAATGGCC[CG
]CAAGGTTTGGGGCAG[CG]G[CG]TTGCAGGAGATGAGCTCAG[
[CG]]CAAAGGGAACCC[CG]CAG[CG]G[CG]AGTG[[CG]]GC
TGCTGGCCTG[CG][CG]CTGTGGCCCCAACAGGCTGGCAGGG[C
G][CG]GG[CG]GGTGG[CG]GGGTTG[CG]GTATGAGCTTTG
SEQ ID NO: 5362
CTGGTAA[CG]A[CG]ACAGCAC[CG]GC[CG]CC[CG]GCCACC
[CG]GTCCCCCTCCATGCT[CG]CT[CG]GC[CG]TG[CG]CTTA
CC[CG]CTGCACAA[CG][CG]TCC[CG]CCC[CG]GCCTCCAGC
CCACTCTC[CG][CG]C[CG][CG]CCAGCCT[CG]AACCTGGAT
CTC[CG][CG]GG[CG]CCTGGG[CG]GGG[CG]GGACTCCAGGC
CCACCCAGCAAGCTAATTGGCTGAGGCCCT[CG]GCTGGGGACCA
ATCAC[CG]GCTCTGAGAAAGTT[CG]GCAGTGGCCACCACATCT
GGTTCT[CG]TTAACTTTTCTAAGGCAG[[CG]]GC[CG]CTGGA
GCAG[[CG]]GGGCTGG[CG]GGGTAAAAGCTCCTGGCCAGGGCT
GCCTGGAGCTGCCCCTTCCACTC[CG]CCCCCAGGGAGCTCC[CG
]GGTCATCCTCTCATC[CG]GGCTGCCC[[CG]][CG]GCCCCCA
AGGAGCCCCACCCC[CG]GGACCAAATGGCC[CG]CAAGGTTTGG
GGCAG[CG]G[CG]TTGCAGGAGATGAGCTCAG[[CG]]CAAAGG
GAACCC[CG]CAG[CG]G[CG]AGTG[CG]GCTGCTGGCCTG[CG
][CG]CTGTGGCCCCAACAGGCTGGCAGGG[CG][CG]GG[CG]G
GTGG[CG]GGGTTG[CG]GTATGAGCTTTGCTCCCTGCCCT
SEQ ID NO: 5363
TTGTGG[CG]A[CG]CCATTTCCCTG[CG]CCCCCAG[CG][CG]
GC[CG]CTTGGCTTTTGTTTGAGGTGA[CG]CTGGG[CG]GCAGC
ATCCTTCCCTCAGCCCTGGGGACCAG[CG]GGGACTACAGAACCC
AGAAGGTTTTGTCTCCAGC[[CG]]CAGGGATGCTGAGCCAATC[
[CG]][CG]CTGAGAAAAGGGT[CG]TTTC[[CG]]CTTTGGGAC
CAATGGGT[CG]GGGAGGGACTTC[CG]GTATCACTTCAGTGG[[
CG]]GTCATTTTTGCAG[CG]CTTGGGTGCATCCAGAC[CG]TCA
GAGCTTTGGGAG[CG]CTTTGTTTGG[[CG]]ACAGT[CG]GAAG
G[CG][CG]AGGGGAGGGGTCCTCC[CG]CTGAACAGTGGGGGTT
CTAAGGGT[CG]G[CG]G[CG]G[CG]GGGTTGA[[CG]]GCTTT
GCCTAGGTCCCTC[CG]CC[CG]TAGCTGT[CG]GGTCC[CG]GC
CC[CG]CTCTGCCCACAGACTC[CG]ATGGCTG[CG]GC[CG][C
G]CTGAGGGCCC[CG]ACTCAGGTGAG[CG]CTGCCTCTACTGGG
CCTCACCCTCCATCCCCAAATTAGTGCCTTCTTGGGTCACTA[CG
]GT[CG]AGATCCTCATGTCCAGTACAGTGGGGGCT[CG]TGGGT
GGGGTCCCTATTTCCAGAACTTGGG
SEQ ID NO: 5364
TGGAAGTGCTGGAAGAAGG[CG]GAGATG[CG]AGTGATCTGCAG
GCTCAGGCACCTGGAGG[CG]CAG[CG]AG[CG][CG]CTGGA[C
G]CTCC[CG]GCAGACAG[CG]ACT[CG]TCCAGC[CG]C[CG][
CG]CAGCAGC[CG][CG]C[CG]GGGC[CG]GC[CG]CCAGGCAG
C[CG]CTGGAGGC[CG]CCAGCCAGAGGCAGAGGGTCAGGAC[CG
][CG]C[CG]GGCACCC[CG]GGCACCATGGCTG[CG]GGT[CG]
AGGG[CG]AGGG[CG]AGGG[CG]C[CG]GG[CG][CG]GGC[CG
]AGGCTCCCTGCTC[CG][CG]C[CG]GGGCTGCACTGCTGAGGA
CCAGGCAGA[CG]C[CG][CG]ACTGAGC[CG]GAAAGTTCAATC
CTTCAACTC[CG]GC[CG]GACCCTCCT[CG]C[CG]ACCTCCCT
CCCAGCT[CG]CCCT[CG]AGGGGTGGGGTGAGGGGGACAGGAGG
C[CG]CACCATTGGCTGGAAGG[CG]TGTGAGTGACAGGGGGGC[
[CG]]GG[CG]CAGGGCCCATGGGC[CG]GTCT[[CG]]TCAGCT
G[CG]AGCC[CG]GG[CG]CTC[CG]GGGGAGGG[CG]A[CG]C[
CG]AG[CG]GCTTTCCA[CG]C[CG][[CG]]CAGGTCT[CG]AG
GGGCCCAGCTCCCA[CG]CAGG[CG]CCTCTCC[CG]TT[CG]CA
CCAAC[CG]CTTCCTGCCCCTCCC
SEQ ID NO: 5365
TT[CG]GGTCTCTCCTGGCTGTGCAAG[CG]ATGGGCCAGAGGGC
TGGA[CG]AGGCTGGCTCCCAATCCAGAATCC[CG]AGCTGGGGA
GTTCTCTCTAGGTTCTGGGGCTCCTGGGAC[CG]GGGCTTTGGGG
AAGGGAGAT[CG]CAGGA[CG]GATATCCAGGAAGGGAGGGGGCC
CTGGGTCTCC[CG]TTACCTG[CG]CCTGC[CG][CG]CTGGGAC
TC[CG]CTCTGGGGGCTGCAGCT[CG]AAGCTC[CG]ACAAGTCT
CCA[CG]TCCAGCC[CG]AGAGGGGG[CG]GC[CG]GGCC[CG]G
CACAG[CG]AGG[CG]AGGAC[CG][CG]AG[CG]CCTCCTGGCA
C[CG]AGGCC[CG]C[CG]GCCCCCCACACTC[CG]CTCATGCCC
TAGACACCCAGGCTTGC[CG]AGCAGCAGCT[CG]AACC[CG][C
G]CCC[CG]GC[CG]CACTGGGG[CG]GGGCA[[CG]]A[[CG]]
GCCCCAAGAAGACCCTCCCTGCAG[[CG]]GCCCCCTG[CG]GCT
C[CG]CTCTGCTTTTAATCACTTCCC[CG]CAGGGTCCC[CG]CC
CCTCA[CG]TGGCATCC[CG][CG]GCAGCTGG[CG]AGTCTGG[
CG]CCT[CG][CG]GC[CG]CCCCCTCTACACTCAGGGACTGGCC
TGAGTC[CG]GGGGGTGGGGGTTT[[CG]]AGG[[CG]]GG
SEQ ID NO: 5366
GC[CG]TG[CG]CTTACC[CG]CTGCACAA[CG][CG]TCC[CG]
CCC[CG]GCCTCCAGCCCACTCTC[CG][CG]C[CG][CG]CCAG
CCT[CG]AACCTGGATCTC[CG][CG]GG[CG]CCTGGG[CG]GG
G[CG]GGACTCCAGGCCCACCCAGCAAGCTAATTGGCTGAGGCCC
T[CG]GCTGGGGACCAATCAC[CG]GCTCTGAGAAAGTT[CG]GC
AGTGGCCACCACATCTGGTTCT[CG]TTAACTTTTCTAAGGCAG[
[CG]]GC[CG]CTGGAGCAG[[CG]]GGGCTGG[CG]GGGTAAAA
GCTCCTGGCCAGGGCTGCCTGGAGCTGCCCCTTCCACTC[CG]CC
CCCAGGGAGCTCC[CG]GGTCATCCTCTCATC[CG]GGCTGCCC[
[CG]][CG]GCCCCCAAGGAGCCCCACCCC[CG]GGACCAAATGG
CC[CG]CAAGGTTTGGGGCAG[CG]G[CG]TTGCAGGAGATGAGC
TCAG[CG]CAAAGGGAACCC[CG]CAG[CG]G[CG]AGTG[CG]G
CTGCTGGCCTG[CG][CG]CTGTGGCCCCAACAGGCTGGCAGGG[
CG][CG]GG[CG]GGTGG[CG]GGGTTG[CG]GTATGAGCTTTGC
TCCCTGCCCTGGGGTCC[CG]GG[CG]CTCCTGGCTCTGGAGGCC
TGGCCAT[CG]GTC[CG]AAAAGCAAAGGGA
SEQ ID NO: 5367
GA[CG]CCATTTCCCTG[CG]CCCCCAG[CG][CG]GC[CG]CTT
GGCTTTTGTTTGAGGTGA[CG]CTGGG[CG]GCAGCATCCTTCCC
TCAGCCCTGGGGACCAG[CG]GGGACTACAGAACCCAGAAGGTTT
TGTCTCCAGC[CG]CAGGGATGCTGAGCCAATC[[CG]][CG]CT
GAGAAAAGGGT[CG]TTTC[CG]CTTTGGGACCAATGGGT[CG]G
GGAGGGACTTC[CG]GTATCACTTCAGTGG[CG]GTCATTTTTGC
AG[CG]CTTGGGTGCATCCAGAC[CG]TCAGAGCTTTGGGAG[CG
]CTTTGTTTGG[CG]ACAGT[CG]GAAGG[CG][CG]AGGGGAGG
GGTCCTCC[CG]CTGAACAGTGGGGGTTCTAAGGGT[CG]G[CG]
G[CG]G[CG]GGGTTGA[[CG]]GCTTTGCCTAGGTCCCTC[CG]
CC[CG]TAGCTGT[CG]GGTCC[CG]GCCC[CG]CTCTGCCCACA
GACTC[CG]ATGGCTG[CG]GC[CG][CG]CTGAGGGCCC[CG]A
CTCAGGTGAG[CG]CTGCCTCTACTGGGCCTCACCCTCCATCCCC
AAATTAGTGCCTTCTTGGGTCACTA[CG]GT[CG]AGATCCTCAT
GTCCAGTACAGTGGGGGCT[CG]TGGGTGGGGTCCCTATTTCCAG
AACTTGGGTGATGGG
SEQ ID NO: 5368
[CG]ACTCCC[CG]TGTGGGAAGAGCT[CG]TCTGGGTGCAG[CG
]C[CG][CG]GCC[CG]CAACATTAGCAA[CG]GCAGCAGCAGTA
GCACTGGTAA[CG]A[CG]ACAGCAC[CG]GC[CG]CC[CG]GCC
ACC[CG]GTCCCCCTCCATGCT[CG]CT[CG]GC[CG]TG[CG]C
TTACC[CG]CTGCACAA[CG][CG]TCC[CG]CCC[CG]GCCTCC
AGCCCACTCTC[CG][CG]C[CG][CG]CCAGCCT[CG]AACCTG
GATCTC[CG][CG]GG[CG]CCTGGG[CG]GGG[CG]GGACTCCA
GGCCCACCCAGCAAGCTAATTGGCTGAGGCCCT[CG]GCTGGGGA
CCAATCAC[[CG]]GCTCTGAGAAAGTT[CG]GCAGTGGCCACCA
CATCTGGTTCT[CG]TTAACTTTTCTAAGGCAG[[CG]]GC[CG]
CTGGAGCAG[CG]GGGCTGG[CG]GGGTAAAAGCTCCTGGCCAGG
GCTGCCTGGAGCTGCCCCTTCCACTC[CG]CCCCCAGGGAGCTCC
[CG]GGTCATCCTCTCATC[CG]GGCTGCCC[[CG]][CG]GCCC
CCAAGGAGCCCCACCCC[CG]GGACCAAATGGCC[CG]CAAGGTT
TGGGGCAG[CG]G[CG]TTGCAGGAGATGAGCTCAG[[CG]]CAA
AGGGAACCC[CG]CAG[CG]G[CG]AGTG[[CG]]GCTGCTGGCC
SEQ ID NO: 5369
C[CG][CG]CATCAGG[[CG]]CTGTTGTTGGAGC[CG]GAACAC
[CG]TG[CG]ACTCTGAC[CG]AAC[CG]GCCCCCTCCT[CG][C
G]CACACACT[CG]C[CG]AGC[CG][CG][CG][CG]CCCCTC[
CG]C[CG]TGACAGTGGC[CG]TGGCCTC[CG]CTCTCT[CG]GG
GCACC[CG]GCAGCCAGAG[CG]CAG[CG]AGAG[CG]GG[CG]G
T[CG]CCAGGGTCCCCTCCCCAGCCAGTCCCAGG[CG]CC[CG]G
TGCACTATG[CG]GGGCA[[CG]]TG[CG]CCCCCCAGCTCTAAT
CTG[CG][CG]CTGACAGGAGCATGATCTGTGCCCAGGCCAGGGC
TGCCAAGGTAAG[[CG]]GG[CG]TAG[CG][CG]GGGACACTGT
CTGC[CG]CCCCTTCCCCCC[CG]CCCTTCTCTGGG[CG]GCTTC
CC[CG]C[CG]CA[CG][CG]AGGCCC[CG]GCAG[CG]CCCTCC
CTTCT[CG]GGGC[CG]AGATCCACCCTCCTCCCCACCCTCT[CG
]CTTCTCC[CG][CG]AGGTTCAATTGTCAGCCTGGGT[CG][CG
]C[CG]C[CG]CC[CG]GTC[CG]GGC[CG]GCATCCC[CG]GGT
GG[CG]ACCC[CG]GCTTGGGTACTGCAGG[CG]CACC[CG]GCC
CTCCTG[CG]GGGTA[CG]GAGAGAAACAAAGAAGCCCCCAAG[C
G]GGTT[CG]CAG[CG]CCTCCCC
SEQ ID NO: 5370
GGTACCCACAGAGGAGCAGGCAGGGAGGGAGGGGATGCAAG[CG]
GGAGGATAAAG[CG]ATGAAGTGTGCTG[CG]TTAC[CG][CG]C
ATCAGG[CG]CTGTTGTTGGAGC[CG]GAACAC[CG]TG[CG]AC
TCTGAC[CG]AAC[CG]GCCCCCTCCT[CG][CG]CACACACT[C
G]C[CG]AGC[CG][[CG]][CG][CG]CCCCTC[CG]C[CG]TG
ACAGTGGC[CG]TGGCCTC[CG]CTCTCT[CG]GGGCACC[CG]G
CAGCCAGAG[CG]CAG[CG]AGAG[CG]GG[CG]GT[CG]CCAGG
GTCCCCTCCCCAGCCAGTCCCAGG[CG]CC[CG]GTGCACTATG[
CG]GGGCA[CG]TG[CG]CCCCCCAGCTCTAATCTG[CG][CG]C
TGACAGGAGCATGATCTGTGCCCAGGCCAGGGCTGCCAAGGTAAG
[CG]GG[CG]TAG[CG][CG]GGGACACTGTCTGC[CG]CCCCTT
CCCCCC[CG]CCCTTCTCTGGG[CG]GCTTCCC[CG]C[CG]CA[
CG][CG]AGGCCC[CG]GCAG[CG]CCCTCCCTTCT[CG]GGGC[
CG]AGATCCACCCTCCTCCCCACCCTCT[CG]CTTCTCC[CG][C
G]AGGTTCAATTGTCAGCCTGGGT[CG][CG]C[CG]C[CG]CC[
CG]GTC[CG]GGC[CG]GCATCCC[CG]GGTGG[CG]ACCC[CG]
GCTTGGGTACTG
SEQ ID NO: 5371
T[CG]GT[CG]CTGTTGGACTCCCT[CG]CCTGC[CG]CTTCTGC
[CG]GATCAAGGAGCTGGCTAT[CG]C[CG]CAGCCATAGCTGCT
CAG[[CG]]AGGGCCTCAGGCCCCAGCCTCTACTG[CG]CCCTC[
CG]GCTTG[CG]CTC[CG]C[CG]GGG[CG]AGGGCAGGACCTGG
G[CG]GCCAGGGAAAGGGCAGT[CG][CG]GGGAGGCAGTGCTAA
AATTTGAGGAGGCTGCAGTAT[CG]AAAACC[CG]G[CG]CTCAC
AAGGTTAGTCAAAGTCTGGGCAGTGG[CG]ACAAAATGTGTGAAA
ATCCAGATGTAAACTTCCCCAACCTCTGG[[CG]]GC[CG]GGGG
G[CG]GGG[CG]GGG[CG]GTCCCAGGCCCTCTTG[CG]AAGTAG
A[CG]TTTGCACCCCAAACTTGCACCCCAAGG[CG]AT[CG]G[C
G]TCCAAGGGGCAGTGGGGAGTTTAGTCACACTG[CG]TT[CG]G
GGTACCAAGTGGAAGGGGAAGAA[CG]ATGCCCAAAATAACAAGA
[CG]TGCCTCTGTTGGAGAGG[CG]CAAG[CG]TTGTAAGGTGTC
CAAAGTATACCTACACATACATACATAGAAAACC[CG]TTTACAA
AGCAGAGTCTGGACCCAGG[CG]GGTAG[CG][CG]CCCC[CG]G
TAGAAAATACTAA
SEQ ID NO: 5372
TGAACACCC[CG]AGGA[CG]TGCCTCT[CG]CACAGGGAG[CG]
CC[CG]TCTTTGCTGGGGCTGGAG[CG]G[CG]CTTGGAGGC[CG
]ACACT[CG]GT[CG]CTGTTGGACTCCCT[CG]CCTGC[CG]CT
TCTGC[CG]GATCAAGGAGCTGGCTAT[CG]C[CG]CAGCCATAG
CTGCTCAG[[CG]]AGGGCCTCAGGCCCCAGCCTCTACTG[CG]C
CCTC[CG]GCTTG[CG]CTC[CG]C[CG]GGG[CG]AGGGCAGGA
CCTGGG[CG]GCCAGGGAAAGGGCAGT[CG][CG]GGGAGGCAGT
GCTAAAATTTGAGGAGGCTGCAGTAT[CG]AAAACC[CG]G[[CG
]]CTCACAAGGTTAGTCAAAGTCTGGGCAGTGG[CG]ACAAAATG
TGTGAAAATCCAGATGTAAACTTCCCCAACCTCTGG[CG]GC[CG
]GGGGG[CG]GGG[CG]GGG[CG]GTCCCAGGCCCTCTTG[CG]A
AGTAGA[CG]TTTGCACCCCAAACTTGCACCCCAAGG[CG]AT[C
G]G[CG]TCCAAGGGGCAGTGGGGAGTTTAGTCACACTG[CG]TT
[CG]GGGTACCAAGTGGAAGGGGAAGAA[CG]ATGCCCAAAATAA
CAAGA[CG]TGCCTCTGTTGGAGAGG[CG]CAAG[CG]TTGTAAG
GTGTCCAAAGTATACCTAC
SEQ ID NO: 5373
CTGGCTCAGCTCTGCA[CG]CCCCCCAGCTAGCTCC[CG]CTCTA
GTCACCTCTTCCC[CG][CG]CCCC[CG]CCCCTCTAGAGCT[CG
][CG]CC[CG]CCCCCCCCCACC[CG][CG]CCTCCTCTGCC[CG
]CCCTCCTCCCCCTCCCCCTTCCCTCCTGCCCTCCCTTCATTCCA
CAAGTGT[CG]CTT[CG]CTCTCTTCAG[CG]CACTTGG[CG]AG
CTGGTGAGGTGAGAGGGATACTG[CG]AGAGGGCTAGGGG[CG]G
CCAGTC[CG]GGGCTGC[[CG]]GCCAAGGCTAGG[CG]CTGCCA
GCC[CG]ATTCTCCTCTGCTCTCCACCCC[CG]CCTCACCCAGTG
GC[CG]GC[CG]GACCTGCACCC[CG][CG]CTTGACCC[CG]CT
CATCCTCCCCC[CG]CC[CG]GC[CG]GG[CG][CG]CTCCTCCC
[CG]GC[CG]GCCCCT[CG]G[CG][CG][CG]G[CG][CG]CTG
GAGG[CG]AA[CG][CG]GGCTGAGG[CG]AA[CG][CG]GGCTG
AGC[CG]AG[CG]CAGTGGC[CG]C[CG]ACCAC[CG]AG[CG]C
CC[CG][CG]C[CG]CTCCCTGCATGTG[CG]GCC[CG][CG]G[
CG]GCT[CG]CAGCTCC[CG]GCAGCAGCCT[CG]GCAGCTT[CG
]GC[CG][CG]CCT[CG]AGAGG[CG]GC[CG]CAGAGGCTCCAG
[CG]G[CG]GC[CG]AG[CG]GC[CG]A
SEQ ID NO: 5374
GGACTCTCTTTACTT[CG]GCTAGGCAC[CG]GCT[CG]GTTCAT
CC[CG]CA[CG][CG]GGG[CG]G[CG]GGGCTGGGCTGGGAAAA
GGTCTTAG[CG]GGGTGCAAACTCAGAGAGCCAG[CG]CTGTTTG
GGAAAGCAGCAGGTTAAAGATGCTGAGT[CG]GGTTGGAAGGGCT
TCTCT[CG]GGCAG[CG]TATGA[CG]GCTTGAGGGAGGGCACCA
GGCACAGGGAGCAGGACTTGG[CG]CTGCTCAGGCTGCACTCTCC
A[CG][CG]GGCAC[CG]TAAGGC[CG]GG[CG][CG]AC[CG]G
GAGGCCC[CG]AGAGAA[CG]CC[CG]GCCCCAGA[CG]CTGGAG
CTGCTCTCTGCCCCCTGCTGTTGCCTCTAC[CG]CAAATGGGTTT
GAGACTGGCAGAGTCAAAAGCC[CG]GCTGGCTTGGGAAAACT[C
G]GGCCAAGCAAAGGGAATGTCCTTTAAGGGTGAAACCTGAAATC
CAGCCC[CG]CATGATCC[[CG]]CACAGG[CG]AGCTGCATC[C
G]GGAGG[CG][CG]AGCTTCAGTG[CG]GAGGCCAGTGTCACCT
TAGAGGAGCTCTCCCTATTGGCTCTTAAA[CG]CCCAGAAAAGTC
CAGTTTCCCTTCCCCATCCTCAC[CG]ACCAACCT[CG][CG]AC
CCATGAA
SEQ ID NO: 5375
GGCTCACAGGCAGTCACACACCTATA[CG]GGCTGACAC[CG]CT
AAGTGAACAGGTGCAGACACAATGGCACT[CG]GGGA[CG]GGCT
CACA[CG][CG]GTCACACACCTGCAGGAACA[CG][CG]CACAC
TACACAC[CG]TTGCTCT[CG]CCTCC[CG]GTATCACCTT[CG]
AA[CG][CG]CTGGC[CG]GCCCCCTCCTAGCCTGGTGCAGT[CG
]GCTC[CG]CTTCTACTCAGTCT[CG]GGTCCTCCTGGGCCAA[C
G]CAGACCCTGGTGCCTCCTTCCTTAGA[CG]AGCTAAGGTCAAG
ACTTCTGCTGCTG[CG]GAGGAGCC[CG][CG]CACCAGGC[CG]
GGGTGGACTG[CG]CCTG[CG]CAT[CG]GGCC[CG]CCTGGTTC
TTAGGAACTA[CG]ACTCCCAGGGGG[CG]CTG[CG][CG]CT[[
CG]]C[CG]GCAC[CG][[CG]]ACTAAAGCTTGCAAAGCCTGGG
TCAT[[CG]]AAACTACAACTCCCAGAAGGCTAAG[[CG]]TCTG
CACA[CG]CTAGGTC[CG]CAGGG[CG]CTGACCCCTGGGGGCC[
CG]AGGAG[CG]C[CG]GGCTGCTGCCC[CG]AGCCC[CG]CCCA
CTACAGCCTCAGTGCATGG[CG]GTGGG[CG]CTTCCAGTAACTC
AGGGGTGGGACTTTTTTCTTGCTCAGGGG
SEQ ID NO: 5376
AGGGGCTTTCCAGA[CG]TGAGTTATCTTTACCTTTCCTTGACTA
GCTAATTCAAGACAACTAGGTAGAAGCCCTTTCTCAGCCTCCCCT
CTT[CG]CTCATCTTT[CG]AGTTCTTGGCCACCCCAGTTCAAAC
ACCAGCACCATTGCCCTCCTCTCAGGTGGCTGCTGCTTAATTTCC
CCTCCACTTTAGTAGTTCTTCTCTTGCCT[CG]CTAGAAGGACTG
GTGTTGGGTGCTTGGAATTCTGGCTATTTTCCTCCTGC[CG]TTC
[CG]ACT[CG]GCACCAGAGTCTGTCTCTACTGAGAA[CG]CAG[
CG][CG]TCAGGGC[CG]AGCTCTTCACTGGCCTGCTC[CG][CG
]CTCTTCAATGCCAG[CG]CCAGG[CG]CTCACCCTGCAGAG[CG
]TCC[CG]CCTCTCAAAGAGGGGTGTGACC[CG][CG]AGTTTAG
ATAGGAGGTTCCTGC[CG]TGGGGAACACCC[CG]C[CG]CCCT[
CG]GAGCTTTTTCTGTGG[CG]CAGCTTCTC[CG]CC[CG]AGC[
CG][CG][CG][CG]GAGCTGC[CG]GGGGCTCCTTAGCACC[CG
]GG[CG]C[CG]GGGCCCT[CG]CCCTTC[CG]CAGCCTTCACTC
CAGCCCTCTGCTCC[CG]CA[CG]CCATGAAGT[CG]C[CG]TTC
TAC
SEQ ID NO: 5377
GAGC[CG]C[CG]AGCTCCTCCC[CG]CC[CG]CCCCCACACCCC
ACCCCCAC[CG]CTGCACTAACCC[CG]GCTCAGCTGGCTC[CG]
[CG]CACTTGCT[CG]GAGGAGC[CG]GGGC[CG]AG[CG]GAC[
CG]C[CG]GCTGCAGGCAG[CG]AG[CG][CG]GCTGGGCTG[CG
]GGGCTGCTTCCC[CG][CG]TCCTC[CG]GGCC[CG]GGC[CG]
CCCTCCTCC[CG]CACAGTG[CG]GAGCAGGGAGGCCC[CG][CG
]CCT[CG]ACCACC[CG][CG]CC[CG]AG[CG]TC[CG][CG]C
CTCCTCCTC[CG]CTCTGCAGG[CG]GGGAC[CG]CC[CG]G[CG
]CT[CG]GCACC[CG]GCAG[CG][CG]GCCCCCTCCAGCCCC[C
G]GCTCC[CG]GCAGCAGAAGCAGAAGGCAG[CG]CCAGGGGC[C
G]C[CG]C[CG]C[CG]C[CG]AGCTC[CG][CG]GGGCT[CG]G
GAGC[CG]GCCC[CG]G[CG]AGGAGG[CG][CG]GAACCATGGC
[CG]ATGGGGG[CG]AGGG[CG]AAGA[CG]AGATCCAGTTCCTG
[CG]AACTGTAAG[CG]C[CG]TG[CG]T[CG][CG]TGTGCTGT
CAGGGGAAGGGGG[CG]TCAGGGCATCCACTAG[CG]GGGTC[CG
]GGCAGAGTGACAG[[CG]]GGCAG[CG]GGGACT[CG][CG]GG
[CG]GGG[CG]AGGGGGTGCCCCCTGAGGATG[[CG]]GGAG
SEQ ID NO: 5378
TCCTTGACTAGCTAATTCAAGACAACTAGGTAGAAGCCCTTTCTC
AGCCTCCCCTCTT[CG]CTCATCTTT[CG]AGTTCTTGGCCACCC
CAGTTCAAACACCAGCACCATTGCCCTCCTCTCAGGTGGCTGCTG
CTTAATTTCCCCTCCACTTTAGTAGTTCTTCTCTTGCCT[CG]CT
AGAAGGACTGGTGTTGGGTGCTTGGAATTCTGGCTATTTTCCTCC
TGC[CG]TTC[CG]ACT[CG]GCACCAGAGTCTGTCTCTACTGAG
AA[CG]CAG[[CG]][[CG]]TCAGGGC[CG]AGCTCTTCACTGG
CCTGCTC[CG][[CG]]CTCTTCAATGCCAG[CG]CCAGG[CG]C
TCACCCTGCAGAG[[CG]]TCC[CG]CCTCTCAAAGAGGGGTGTG
ACC[CG][CG]AGTTTAGATAGGAGGTTCCTGC[CG]TGGGGAAC
ACCC[[CG]]C[CG]CCCT[CG]GAGCTTTTTCTGTGG[CG]CAG
CTTCTC[CG]CC[CG]AGC[CG][CG][CG][CG]GAGCTGC[CG
]GGGGCTCCTTAGCACC[CG]GG[CG]C[CG]GGGCCCT[CG]CC
CTTC[CG]CAGCCTTCACTCCAGCCCTCTGCTCC[CG]CA[[CG]
]CCATGAAGT[CG]C[CG]TTCTAC[CG]CTGCCAGAACACCACC
TCTGTGGAAAAAGGCAA
SEQ ID NO: 5379
TCAGGGGCTTTCCAGA[CG]TGAGTTATCTTTACCTTTCCTTGAC
TAGCTAATTCAAGACAACTAGGTAGAAGCCCTTTCTCAGCCTCCC
CTCTT[CG]CTCATCTTT[CG]AGTTCTTGGCCACCCCAGTTCAA
ACACCAGCACCATTGCCCTCCTCTCAGGTGGCTGCTGCTTAATTT
CCCCTCCACTTTAGTAGTTCTTCTCTTGCCT[CG]CTAGAAGGAC
TGGTGTTGGGTGCTTGGAATTCTGGCTATTTTCCTCCTGC[CG]T
TC[CG]ACT[CG]GCACCAGAGTCTGTCTCTACTGAGAA[CG]CA
G[[CG]][[CG]]TCAGGGC[CG]AGCTCTTCACTGGCCTGCTC[
CG][[CG]]CTCTTCAATGCCAG[CG]CCAGG[CG]CTCACCCTG
CAGAG[[CG]]TCC[CG]CCTCTCAAAGAGGGGTGTGACC[CG][
CG]AGTTTAGATAGGAGGTTCCTGC[CG]TGGGGAACACCC[CG]
C[CG]CCCT[CG]GAGCTTTTTCTGTGG[CG]CAGCTTCTC[CG]
CC[CG]AGC[CG][CG][CG][CG]GAGCTGC[CG]GGGGCTCCT
TAGCACC[CG]GG[CG]C[CG]GGGCCCT[CG]CCCTTC[CG]CA
GCCTTCACTCCAGCCCTCTGCTCC[[CG]]CA[CG]CCATGAAGT
[CG]C[CG]TTCTAC
SEQ ID NO: 5380
AGATCTGGG[CG]G[CG]G[CG]GC[CG]AAAGCAG[CG]A[CG]
[CG]CC[CG]GAGCATCCCTTG[CG]ATA[CG]CTAGGGGA[CG]
GTGCTTTCTCTGTCCCAGTTG[CG]TG[CG]G[CG]GGGCTGGGG
CCCAGGC[[CG]]CCCCAAATCTCCCCCACTAGAGTGACCAC[CG
]CACAGTTGTCCC[CG]CTGGG[CG][CG]CTCCTC[CG]GTGTC
TG[CG]CTCAGC[CG]CTCTCCTCTTCTCTCTCC[CG]CC[CG]C
C[CG]CAG[CG]CCATGGTCTGGCAGTGTGTTTAG[[CG]]CTCA
CCACCATGTGCACCAGCTTGTTGCTAGTGTACAGCAGCCT[CG]G
[[CG]]GCCAGAAGGAG[CG]GCCCC[CG]CAGCAGCAGCAGCAG
CAGCAGCAACAGCAGCAGCAGG[CG]T[CG]GCCAC[CG]GCAGC
T[CG]CAGC[CG]G[CG]G[CG]GAGAGCAGCACCCAGCAG[CG]
CCC[CG]GGGTCCC[CG][CG]GGAC[CG][CG]GCCACTGGA[C
G]GATACCT[CG]GAGTGG[CG]GACCACAAGGTGACAGCATGCC
[CG]CCAGCC[CG]CT[CG]CCACTCAGCCTGGGGATCC[CG]CA
CACCTGAGCCTTCCCCCTTTCC[CG]GGGCTGGGAGG[CG]CTGT
GAGTAGGTGC[CG]TT[CG]AAGGCTGGAGGGGAGTGGACC
SEQ ID NO: 5381
TGCAAGGAT[CG]CAAGGATCCAGGGCCCCAGGAAAGGAGGGGTG
TGAAGGACTCAAAATTCCAGCAGCTTGGCTGGGGTGGCTG[CG]C
CAGA[CG]GGCCCTTCCCCAAAGTGCAAACCCACCCCTGTCCT[C
G]GCCC[CG]G[CG][CG]CTCCCTCCCTCAGCC[CG]GGGC[CG
]TACACCACCTGCCCTCTAC[CG]AGAGATCTGGG[CG]G[CG]G
[CG]GC[CG]AAAGCAG[CG]A[CG][CG]CC[CG]GAGCATCCC
TTG[CG]ATA[CG]CTAGGGGA[CG]GTGCTTTCTCTGTCCCAGT
TG[CG]TG[CG]G[CG]GGGCTGGGGCCCAGGC[[CG]]CCCCAA
ATCTCCCCCACTAGAGTGACCAC[CG]CACAGTTGTCCC[CG]CT
GGG[CG][CG]CTCCTC[CG]GTGTCTG[CG]CTCAGC[CG]CTC
TCCTCTTCTCTCTCC[CG]CC[CG]CC[CG]CAG[CG]CCATGGT
CTGGCAGTGTGTTTAG[[CG]]CTCACCACCATGTGCACCAGCTT
GTTGCTAGTGTACAGCAGCCT[CG]G[CG]GCCAGAAGGAG[CG]
GCCCC[CG]CAGCAGCAGCAGCAGCAGCAGCAACAGCAGCAGCAG
G[CG]T[CG]GCCAC[CG]GCAGCT[CG]CAGC[CG]G[CG]G[C
G]GAGAGCAGCACCCAGCAG[CG]CCC
SEQ ID NO: 5382
G[CG]CTCCCTCCCTCAGCC[CG]GGGC[CG]TACACCACCTGCC
CTCTAC[CG]AGAGATCTGGG[CG]G[CG]G[CG]GC[CG]AAAG
CAG[CG]A[CG][CG]CC[CG]GAGCATCCCTTG[CG]ATA[CG]
CTAGGGGA[CG]GTGCTTTCTCTGTCCCAGTTG[CG]TG[CG]G[
CG]GGGCTGGGGCCCAGGC[[CG]]CCCCAAATCTCCCCCACTAG
AGTGACCAC[CG]CACAGTTGTCCC[CG]CTGGG[CG][CG]CTC
CTC[CG]GTGTCTG[CG]CTCAGC[CG]CTCTCCTCTTCTCTCTC
C[CG]CC[CG]CC[CG]CAG[CG]CCATGGTCTGGCAGTGTGTTT
AG[CG]CTCACCACCATGTGCACCAGCTTGTTGCTAGTGTACAGC
AGCCT[CG]G[[CG]]GCCAGAAGGAG[CG]GCCCC[CG]CAGCA
GCAGCAGCAGCAGCAGCAACAGCAGCAGCAGG[CG]T[CG]GCCA
C[CG]GCAGCT[CG]CAGC[CG]G[CG]G[CG]GAGAGCAGCACC
CAGCAG[CG]CCC[CG]GGGTCCC[CG][CG]GGAC[CG][CG]G
CCACTGGA[CG]GATACCT[CG]GAGTGG[CG]GACCACAAGGTG
ACAGCATGCC[CG]CCAGCC[CG]CT[CG]CCACTCAGCCTGGGG
ATCC[CG]CACACCTGAGCCTTCCCCCTTTCC[CG]GGGCT
SEQ ID NO: 5383
TTTCTCAGCCTCCCCTCTT[CG]CTCATCTTT[CG]AGTTCTTGG
CCACCCCAGTTCAAACACCAGCACCATTGCCCTCCTCTCAGGTGG
CTGCTGCTTAATTTCCCCTCCACTTTAGTAGTTCTTCTCTTGCCT
[[CG]]CTAGAAGGACTGGTGTTGGGTGCTTGGAATTCTGGCTAT
TTTCCTCCTGC[CG]TTC[CG]ACT[CG]GCACCAGAGTCTGTCT
CTACTGAGAA[CG]CAG[CG][CG]TCAGGGC[CG]AGCTCTTCA
CTGGCCTGCTC[CG][CG]CTCTTCAATGCCAG[CG]CCAGG[CG
]CTCACCCTGCAGAG[CG]TCC[CG]CCTCTCAAAGAGGGGTGTG
ACC[CG][CG]AGTTTAGATAGGAGGTTCCTGC[CG]TGGGGAAC
ACCC[CG]C[[CG]]CCCT[CG]GAGCTTTTTCTGTGG[CG]CAG
CTTCTC[CG]CC[CG]AGC[CG][CG][CG][CG]GAGCTGC[CG
]GGGGCTCCTTAGCACC[CG]GG[CG]C[CG]GGGCCCT[CG]CC
CTTC[CG]CAGCCTTCACTCCAGCCCTCTGCTCC[CG]CA[CG]C
CATGAAGT[CG]C[CG]TTCTAC[CG]CTGCCAGAACACCACCTC
TGTGGAAAAAGGCAACT[CG]G[CG]GTGATGGG[CG]GGGTGCT
CTTCAGCAC[CG]GCCT
SEQ ID NO: 5384
GCCCTCCTCTCAGGTGGCTGCTGCTTAATTTCCCCTCCACTTTAG
TAGTTCTTCTCTTGCCT[CG]CTAGAAGGACTGGTGTTGGGTGCT
TGGAATTCTGGCTATTTTCCTCCTGC[CG]TTC[CG]ACT[CG]G
CACCAGAGTCTGTCTCTACTGAGAA[CG]CAG[CG][CG]TCAGG
GC[CG]AGCTCTTCACTGGCCTGCTC[CG][CG]CTCTTCAATGC
CAG[CG]CCAGG[CG]CTCACCCTGCAGAG[CG]TCC[CG]CCTC
TCAAAGAGGGGTGTGACC[CG][CG]AGTTTAGATAGGAGGTTCC
TGC[CG]TGGGGAACACCC[CG]C[[CG]]CCCT[CG]GAGCTTT
TTCTGTGG[CG]CAGCTTCTC[CG]CC[CG]AGC[CG][CG][CG
][CG]GAGCTGC[CG]GGGGCTCCTTAGCACC[CG]GG[CG]C[C
G]GGGCCCT[CG]CCCTTC[CG]CAGCCTTCACTCCAGCCCTCTG
CTCC[CG]CA[CG]CCATGAAGT[CG]C[CG]TTCTAC[CG]CTG
CCAGAACACCACCTCTGTGGAAAAAGGCAACT[CG]G[CG]GTGA
TGGG[CG]GGGTGCTCTTCAGCAC[CG]GCCTCCTGGGCAACCTG
CTGGCCCTGGGGCTGCTGG[CG][CG]CT[CG]GGGCTGGGGTGG
TGCT[CG][CG]G[CG]TCCACT
SEQ ID NO: 5385
CCCCACTG[CG]C[CG]GG[CG]CTG[CG]GAGAG[CG]G[CG]A
GGGTGGG[CG][CG]AGG[CG]GAGAA[CG][CG]ATGAATGAGT
TCTCCCCT[CG]CCT[CG]GAGTTGTCTGAGTTGG[[CG]]G[CG
]CTG[CG]CCCAGGCTTC[CG]GCTCTCAG[CG]CCCCA[CG][C
G][CG][CG]TGGCTCCC[CG]GGCTGCCACCCA[CG]CC[CG][
CG]GC[CG]GGGC[CG]AGCCAGCCA[CG]CAGGGCAGC[CG]AG
GCTC[CG]GAGCTCCTGTCC[CG]GCCCCAGTC[CG]GGTAAAAG
GAGGGTTGTCCCCAG[CG]GAGG[CG]CACAGC[CG][CG][CG]
TTCTCCCTGCACTCTCTT[CG][CG]GTCCCATCTGTTCCCCATG
G[CG]TCTCATC[CG]CAAACC[CG]GATCCAGGCTTACCTGGAG
AAGAACAAGAT[CG]GTCCCCTGTTTGAGGTAAGG[CG]CTGTGG
AGGAGGGCAGTCC[CG]TTGTCTTTAGGGGAAGGGGTGCAGTAAT
GAAAACAGAACACTCCCAATCCCACCCCTCCCAGGGAAGGAGGGC
TAGAGAACCAA[CG][CG][CG]GGAGAGGG[CG]CCCTGGGATT
CACTGGCATT[CG]CTCTGTCC[CG]GCCAGGTGTCCTGGAA[CG
][CG]GC[CG]GG[CG]GGCACTTAGCCAGTTACCTGAA
SEQ ID NO: 5386
GAG[CG]G[CG]AGGGTGGG[CG][CG]AGG[CG]GAGAA[CG][
CG]ATGAATGAGTTCTCCCCT[CG]CCT[CG]GAGTTGTCTGAGT
TGG[[CG]]G[CG]CTG[CG]CCCAGGCTTC[CG]GCTCTCAG[C
G]CCCCA[CG][CG][CG][CG]TGGCTCCC[CG]GGCTGCCACC
CA[CG]CC[CG][CG]GC[CG]GGGC[CG]AGCCAGCCA[CG]CA
GGGCAGC[CG]AGGCTC[CG]GAGCTCCTGTCC[CG]GCCCCAGT
C[CG]GGTAAAAGGAGGGTTGTCCCCAG[CG]GAGG[CG]CACAG
C[CG][CG][CG]TTCTCCCTGCACTCTCTT[CG][CG]GTCCCA
TCTGTTCCCCATGG[CG]TCTCATC[CG]CAAACC[CG]GATCCA
GGCTTACCTGGAGAAGAACAAGAT[CG]GTCCCCTGTTTGAGGTA
AGG[CG]CTGTGGAGGAGGGCAGTCC[CG]TTGTCTTTAGGGGAA
GGGGTGCAGTAATGAAAACAGAACACTCCCAATCCCACCCCTCCC
AGGGAAGGAGGGCTAGAGAACCAA[CG][CG][CG]GGAGAGGG[
CG]CCCTGGGATTCACTGGCATT[CG]CTCTGTCC[CG]GCCAGG
TGTCCTGGAA[[CG]][CG]GC[CG]GG[CG]GGCACTTAGCCAG
TTACCTGAA[CG][CG]GACAGGTGAGCT[CG]GGAGGG
SEQ ID NO: 5387
C[CG]CCTCT[CG]AACCCTCCTTGT[CG][CG]TTCTTCC[CG]
CCC[CG]CACCTTTCTCCT[CG][CG]TTTCTGGGAAGTCAGAAG
CAAACCA[CG]AGAAAATGCCTT[CG]CAAGGAGCCTCCCTACAC
C[CG]CCCTGTCACTGAAACAAAAG[CG]TAGGGAGGAGGGAGGT
GGGAGAGGAGG[CG]GGAGGAAGGTTTAGAAAGA[CG]ATGGGGA
CAGCAGTAGGAAGGAGCTGGGGAGGGGGGTCAAGGTGGCAAGCAG
AGCTGGGGGCCCAGAGTC[CG]CTCC[CG]TGC[CG][CG][[CG
]]G[CG]AGT[CG]CTTACATGCCCAGGT[[CG]]CTGTAGGTGT
TGAAGAACTCCATCTGATTGCA[[CG]]CCTGAATCCAGCCCACC
ACCCAGGTCT[CG]TGG[CG]GGGGATGGGGGGCATGACCA[CG]
[CG]GG[CG]GAGGCTTTGAAGTAGGGGGTCTTGTAG[CG]CAGG
A[CG]ATGGG[CG]AGGTCTCCT[CG]ATG[CG][CG]TGGGGCA
CTGGT[CG]ATGGTGG[CG]CACACAT[CG]TACACCA[CG]ATG
TTCT[CG][CG]C[CG]GATC[CG][CG]CCTTGCAGGTGATGCT
TTGGATACAGCCCATCCTGCAGCC[CG]GTGC[CG]GCA[CG]G[
CG][CG]G[CG]TGGGGCAG[CG][CG]GGGGC
SEQ ID NO: 5388
GCCC[CG]CACCTTTCTCCT[CG][CG]TTTCTGGGAAGTCAGAA
GCAAACCA[CG]AGAAAATGCCTT[CG]CAAGGAGCCTCCCTACA
CC[CG]CCCTGTCACTGAAACAAAAG[CG]TAGGGAGGAGGGAGG
TGGGAGAGGAGG[CG]GGAGGAAGGTTTAGAAAGA[CG]ATGGGG
ACAGCAGTAGGAAGGAGCTGGGGAGGGGGGTCAAGGTGGCAAGCA
GAGCTGGGGGCCCAGAGTC[CG]CTCC[CG]TGC[CG][CG][[C
G]]G[CG]AGT[CG]CTTACATGCCCAGGT[CG]CTGTAGGTGTT
GAAGAACTCCATCTGATTGCA[[CG]]CCTGAATCCAGCCCACCA
CCCAGGTCT[CG]TGG[CG]GGGGATGGGGGGCATGACCA[CG][
CG]GG[CG]GAGGCTTTGAAGTAGGGGGTCTTGTAG[CG]CAGGA
[CG]ATGGG[CG]AGGTCTCCT[CG]ATG[CG][CG]TGGGGCAC
TGGT[CG]ATGGTGG[CG]CACACAT[CG]TACACCA[CG]ATGT
TCT[CG][CG]C[CG]GATC[CG][CG]CCTTGCAGGTGATGCTT
TGGATACAGCCCATCCTGCAGCC[CG]GTGC[CG]GCA[CG]G[[
CG]][CG]G[CG]TGGGGCAG[CG][CG]GGGGCC[CG][CG][C
G]GGCAGC[CG]GGGG[CG]CC[CG]TCA[CG]C[CG]G
SEQ ID NO: 5389
GCCTCAGCTTCCTGAGCCTGAATCAATGCTTCTAATCA[CG]TGG
G[CG]CTCAGC[CG][CG]A[CG]GGACCACAGAAAT[CG]ACCA
G[CG]GCCTGTTTAAGGAG[CG]ACAAGGCCCAGCCCTCTCCTGC
ATTGTGG[CG]A[CG]CCATTTCCCTG[CG]CCCCCAG[CG][CG
]GC[CG]CTTGGCTTTTGTTTGAGGTGA[CG]CTGGG[CG]GCAG
CATCCTTCCCTCAGCCCTGGGGACCAG[CG]GGGACTACAGAACC
CAGAAGGTTTTGTCTCCAGC[[CG]]CAGGGATGCTGAGCCAATC
[CG][CG]CTGAGAAAAGGGT[CG]TTTC[CG]CTTTGGGACCAA
TGGGT[CG]GGGAGGGACTTC[CG]GTATCACTTCAGTGG[CG]G
TCATTTTTGCAG[CG]CTTGGGTGCATCCAGAC[CG]TCAGAGCT
TTGGGAG[CG]CTTTGTTTGG[[CG]]ACAGT[[CG]]GAAGG[[
CG]][CG]AGGGGAGGGGTCCTCC[CG]CTGAACAGTGGGGGTTC
TAAGGGT[CG]G[CG]G[CG]G[CG]GGGTTGA[[CG]]GCTTTG
CCTAGGTCCCTC[CG]CC[CG]TAGCTGT[CG]GGTCC[CG]GCC
C[CG]CTCTGCCCACAGACTC[CG]ATGGCTG[CG]GC[CG][CG
]CTGAGGGCCC[CG]ACTCAGGTGAG[CG]CTG
SEQ ID NO: 5390
CTCCTCTCAGGTGGCTGCTGCTTAATTTCCCCTCCACTTTAGTAG
TTCTTCTCTTGCCT[CG]CTAGAAGGACTGGTGTTGGGTGCTTGG
AATTCTGGCTATTTTCCTCCTGC[CG]TTC[CG]ACT[CG]GCAC
CAGAGTCTGTCTCTACTGAGAA[CG]CAG[CG][CG]TCAGGGC[
CG]AGCTCTTCACTGGCCTGCTC[CG][[CG]]CTCTTCAATGCC
AG[CG]CCAGG[CG]CTCACCCTGCAGAG[CG]TCC[CG]CCTCT
CAAAGAGGGGTGTGACC[CG][CG]AGTTTAGATAGGAGGTTCCT
GC[CG]TGGGGAACACCC[[CG]][[CG]]CCCT[CG]GAGCTTT
TTCTGTGG[CG]CAGCTTCTC[CG]CC[CG]AGC[CG][CG][CG
][CG]GAGCTGC[CG]GGGGCTCCTTAGCACC[CG]GG[CG]C[C
G]GGGCCCT[CG]CCCTTC[CG]CAGCCTTCACTCCAGCCCTCTG
CTCC[CG]CA[CG]CCATGAAGT[CG]C[CG]TTCTAC[CG]CTG
CCAGAACACCACCTCTGTGGAAAAAGGCAACT[CG]G[CG]GTGA
TGGG[CG]GGGTGCTCTTCAGCAC[CG]GCCTCCTGGGCAACCTG
CTGGCCCTGGGGCTGCTGG[CG][CG]CT[CG]GGGCTGGGGTGG
TGCT[CG][CG]G[CG]TCCACTG
SEQ ID NO: 5391
CTTTTCTTCTCT[CG]CACTCCACCTCCCACTGCCC[CG]CACCC
CCTTTAGCC[CG]GTGTTCCC[CG]CTC[CG]CTATGATTGA[CG
]TCTGGAAAGAAAGAGCTTTGTGAGGGGATGATTGTTATTAACTT
GTTATCCC[CG]G[CG]GGGGGG[CG]GGGAAC[CG]A[CG]TGC
C[CG]GGTGAG[CG]C[CG]GAGACC[CG]GAGCC[CG]GGGAG[
CG][CG]GGA[CG]AGCTACCAG[CG]CT[CG]GGTGG[CG]GC[
CG]CCAG[CG]GCCAG[CG]AAGGAGGCTG[CG][CG]CCAGCC[
CG]CC[CG][CG]G[CG]CC[CG]GGCTCAGG[CG]C[CG]TGA[
CG]GCTGCA[[CG]][CG]CTGCCC[CG]CACTCTGAGGGCCTTC
ATTAGCT[CG]CTCCC[CG][CG]C[CG]AGGCTGGGG[CG]GGC
AGCA[CG]CT[CG]GAACTTCTGATCTGTTTCTCCATACTTTCTC
CCCCTCCTACTC[CG]CAGTGTCAGGGGCTCATGTCAGAGGAGTG
[CG]GG[CG]GACTACAGCCCTGG[CG]GC[CG]GGAGGACT[CG
]CAAAGG[CG]C[CG]GGGAAGAGGGACTGGTGAGTG[CG]GAGG
GGCT[CG][CG]TGCTGGCAAGACTGGC[[CG]]TGGGGAGG[CG
]G[CG]CAGGA[CG]AAGGGC[CG]CTGTCCC[CG][[CG]]CTC
CTTCCAGGGCCC
SEQ ID NO: 5392
[CG]CTGGTAGC[CG]GG[CG]CTGCCATTCACTGCAAGGTCCTC
TG[CG]CCCTGGAATTGC[CG][CG]G[CG]G[CG]GATGCAGAG
GCCAACCCAGAGTGCAACCATGGAGA[CG][CG]A[CG]TGTGTC
CCATAGTAACCTGTTACAACATTATTTATAAGCTGCCATCCCTAG
CCTTCCCC[CG]CTTCCCCTC[CG]CTCCCT[CG]CCAGACTTG[
CG]GG[CG]A[CG]GGCACAGC[CG][CG]TCTGGCTCTTCC[CG
]GCCTCTCCCTTCTCT[CG][CG]GG[CG]CAGC[CG]ATCAATA
GTTAACACC[CG]GCTG[CG]GA[CG]G[CG]GCTCCATC[CG][
CG]GCAATCAC[CG]TAGTGCTTGTTTGTGGAAGC[CG]AG[[CG
]]TG[CG]TG[CG][CG][CG][CG][CG]CACCCAGTCCAG[CG
][CG]GAGTGGG[CG]TCTACC[CG]AGGAGGGGTGTCTGGGGAG
GGGCTGCCCT[[CG]]TTACCCAAACAGTTTG[[CG]]CT[CG]C
TTAACCTTGATGCAGCT[CG]AGGCTTCCCAGTCCAGCTCAGTTC
AGACAGAAAACCTGG[CG][CG][CG][CG][CG][CG]CACACA
CACA[CG]CCTCCCCTGG[CG]T[CG]C[CG]CC[CG]GC[CG]G
GTCCCTGCCCTTAGGGACCAGAG[CG]G[CG]AC[CG]CTGCACC
C[CG]CAC[CG]CCTGCTGGAGGAGCCCC[CG]GAGC[CG]GGGC
[CG]AGC[CG]C[CG]G[CG]TCCC[CG]AGTG[CG]CCCCCTGT
G[CG]TGC[CG]C[CG][CG]C
SEQ ID NO: 5393
GCAAGCAACAACTTTGAGGTGAGCAGCAAGTTGTGCACCTCCTCT
CAAGG[[CG]]CTGGATCAGCACCCCTGAGCTGCCCCCACAGCAC
ACACTCCCAATG[CG]AGG[CG]G[CG]G[CG]GCAGCAACAGAG
GCAGCAGCAGCAAT[CG][CG]GCAGCAG[CG]GCAGAGG[CG]G
[CG]GCAGCTG[CG][CG]CCT[CG]GGCCCAACCC[CG]CT[CG
]GCTCCCCCTCT[CG][CG]G[CG]CCCTGCCTGTCTTG[CG]TG
TG[CG]TGTG[CG]TGTGCTCAGCCTCAG[CG]TGAGGGGCACCT
GCT[[CG]]TCTGGGCTCACAG[[CG]]AAGGCAGCCT[[CG]][
CG][CG]AGCTGC[CG]CTGC[CG]CTGCTGC[CG]CCACTGGTG
TTGC[CG]CTCTCAGG[CG]CCAGGCTCCC[CG]T[CG]C[CG]C
[CG]C[CG]C[CG]CCT[CG]CCAGCCAGAGTTGGGCTC[CG]TG
GGCTTCCCCCCT[CG]CAGCCT[CG]GTCCTTCCC[CG]CTGCCT
GCAAGTCAGCCTGGCTC[CG]AGTCA[CG]TGTCAGTGCCTGAGG
CAGAGACTG[CG]AGAAAAAAA[CG][CG]CTTCATTCCTTCTTC
CAC[CG]CCATACTGTATTTTATACTAAATCTCATTTTTATTCCA
ACATTTTACTCC[CG]CT[CG]GTGAGTACCTT
SEQ ID NO: 5394
GGATGAAGCCCACCC[CG]TCCCCTTCTGGTACCAAAGTGCTTAC
TCCTCTCCAAAGTGC[CG]TGTCTGAACTGC[CG]CTGGGAAGAA
G[CG]GCTCCTGAGA[CG][CG]CCCACACCTTTCACCTGC[CG]
[CG][CG]CTTCCCCCTCCT[CG]GCCACCTTCC[CG]G[CG]GA
AGCAG[CG]AGGAGGGAGCCCCCTTTGGC[CG]TCCTC[CG]TGG
AAC[CG]GTTTTC[CG]AGGCTGGCAAAAGC[CG]AGGCTGGATT
TGGGGGAGGAATATTAGACT[CG]GAGGAGTCTG[CG][CG]CTT
TTCTCCTCCC[CG][CG]CCTCC[CG]GT[CG]C[[CG]][CG]G
GTTCAC[CG]CTCAGTCCC[CG][CG]CT[CG]CTC[CG]CACCC
CACCCACTTCCTGTGCT[CG]CC[CG]GGGGG[CG]TGTGC[CG]
TG[CG]GCTGC[CG]GAGTT[CG]GGGAAGTTGTGGCTGT[CG]A
GAATGGGGGTCTGTGGGTACCTGTTCCTGCCCTGGAAGTGCCT[C
G]TGGT[CG]TGTCTCTCAGGCTGCTGTTCCTTGTACCCACAGGA
GTGCC[CG]TG[CG]CAG[CG]GAGATGCCACCTTCCCCAAAGCT
ATGGACAA[CG]TGA[CG]GTC[CG]GCAGGGGGAGAG[CG]CCA
CCCTCAGGTAGGGAGCTGACATTGT
SEQ ID NO: 5395
TGC[CG]CAGGTGAG[CG]ATGTAACTGGAAGCCAGC[CG]GAG[
CG]TGTCCAGCTTGGAGAGCTTAGTGT[CG]GGGGGCACCCAGGG
CAGGCTGGTCTTGAGCCTGGAGAAGGCTTTGCTCAGCA[CG][CG
]CATC[CG]GGCA[CG]CTCA[CG]GG[CG]TTGGC[CG][CG]T
TC[CG]CTG[CG]ACTGCTTGCACTCTG[CG]GCTGAGCCCTTGG
C[CG]GGAGGGGCTTCTTGCCACCAC[CG]CC[CG][CG]CTACC
ACCTG[CG]C[CG]C[CG]CCCCCAGCCACA[CG]GGGC[CG]CT
TCCTCTTGCAGCCTTC[CG][CG]CTGC[CG]GCTGTGCCCAGAG
[CG]CAG[CG]CTCCTCCT[CG]C[CG]T[CG]GGGTCCTCCTCC
TCTGC[CG]A[CG]AGTTGTCACTGGG[CG]AGG[CG]TAGCTG[
CG]CTCTA[CG]C[CG][CG]GAGGGG[CG]GCCTCTTGGAGG[[
CG]]GGGAC[CG]GGTACTCC[CG]CTGCAGCCCC[CG]AAGCTC
CATCTCCTC[CG]GATCACTCAC[CG]AGCC[CG]TGGACATCC[
CG]TTGTCCCCCTTGCCCACA[CG][CG]TCCTCTTTCCTCCCCC
CTGGCCAGTCT[CG]CTGTCTC[CG]CCTTC[CG]CTCCCTGG[C
G]GAGG[CG]GAGGCCAGAGAG[CG]CTCCAAGGAAGACTAAAAA
CCCAGGC[CG]GGAAG[CG][CG]GGGTGAGAAAG[CG]AGGTGG
GTGG[CG]AGAG[CG]TGAG[CG]CCCCTCTGCTGACCC[CG]GG
GAG[CG]TGGACTA[CG]AGTTGG[CG]CCCAAGTCCAGAATC[C
G][CG][CG]CAC[CG][[CG]]GTAAGCTG[CG]CCTTTTGAAA
AGGC
SEQ ID NO: 5396
GGGT[CG]GCTGTCCTTCCCCACTGGTCACACAAGAG[CG]GC[C
G]TCCTTCCCCAGCT[CG]AAGCC[CG]TAGCACATT[CG]CAGG
CAAAGCCTCCCAAGT[CG]TCTAGGCAGTTAGGGAGCTCTG[CG]
CATTTGCCAGCA[CG]GAGGTACCTCC[CG]GGGCAGGGACACAA
CACAT[CG]CC[CG]AGAGTTTGTCCCAG[CG]AG[CG]C[CG]A
TTT[CG]TC[CG][CG]ATGCAAGTAACTGAGAT[CG]GGAGCTG
TCCC[CG]GCAGAG[CG]CACTCACCT[CG]GTCCCAGGTGGACT
GAAGTCCAGAG[CG]G[CG]CTGTGCAGCTGGAAGGG[CG][CG]
[CG]ATAGCTCAAGTTAGAGG[CG]GCCC[[CG]]GGG[CG][CG
]G[CG]CAGGACACAAGACCTCAAACTGGTACTTGCACAGGTAGC
[CG]TTGG[CG][CG]CAGGTGGCAT[CG]CATCTCCTTCCAGCC
TG[CG]GGCT[CG]ACCCCAC[CG]GTGGCCTGGAGTAC[CG][C
G]CATCTC[CG][CG][CG]GTGCAGGAG[CG]TTGGGGCTCCTC
CACCCACTGCAG[CG]TGT[CG]CTTT[CG]AGAC[CG]C[CG]G
GGT[CG]GAGGACAGCCAGGAGAAACCC[CG]CAAAGGCT[CG]T
TCTCCAGGGTGCAGTGGGAA[CG]CCTG[CG]CTCCAGTG[CG]A
CCCAGAACAGCAGGTCTTTGGAGCCCCCTC[CG]GGCCCTGGGCC
TGCC[CG]CAGGAG[CG][CG]AG
SEQ ID NO: 5397
GGTCTCCAGGG[CG]GAAAGGGGTGGAAGCTGACCCT[CG]CCCT
TCCCTCCAG[CG]CTGGCTTCAGGTGTGCCTTCTGCTACCTCCTG
TACTG[CG]AACAGGGGCC[CG]C[CG]AGCTC[CG]GGAGCCCC
TAGAAGAGGAAGACTCCTCTGGCCCCACTAGGTATCATC[CG][C
G]CTCTCC[CG]CTTTCCACCTG[CG]CCCT[CG]CTTGGGCCAA
TCTCTGC[CG]CA[CG]TGTCCATCCCTGAACTGCA[CG]CTATC
CTCCACCCC[CG]GGGGGTTCCTG[CG]CACTGAAAGAC[CG]TT
CTC[CG]GCAGGTTTTGGGATC[CG]G[CG]A[CG]GCTGAC[CG
][CG][CG]C[CG]CCCCCA[CG]CC[CG]GTTCCA[CG]ATGCT
GCAATACAGAAAGTTTA[CG]T[CG]GCCC[CG]ACC[CG][CG]
[CG]GGACTGCAGGGTC[CG]C[CG]GAG[CG][CG]G[CG]CAG
AGGCTTTTCCTG[CG][CG]TT[CG]GCCC[CG]GGAAAGGGG[C
G]GGAGGGCTGGCTC[CG]GGAG[CG]CA[CG]GG[CG][CG]G[
CG]GGGAGGGTACTCACTGTGAAGCA[CG]CTG[CG]CCCATGGA
TCATGTCTGTG[CG]TTACACCAGAGGCTC[CG]GGCTCCACTAA
TTCCATTTAGAGA[CG]GGAAGACTTCCAGTGG[CG]GG
SEQ ID NO: 5398
CAGCAGCCTGCTGAGGAGTGGGGA[[CG]]AAGAGCAGCCTAAAC
TTAGGGCT[CG]GGATATTT[CG]ATGCCACCCAAATTGC[CG]T
CCTACCCCAA[CG]AGGCAGGGAAAGGAG[CG]GAG[CG][CG][
CG][CG][CG]AGCTGAGTGAGTGCTTA[CG]T[CG]CAG[CG]A
GATCTGTGCTGGGATAATTAGAGAGGAGTTGGGCTGAGC[CG]AG
TCCTCTTTCAGCAGCAGCAGC[CG]GAGC[CG]C[CG]C[CG]CA
GCC[CG]GTGGGGCAACCCTGACT[CG]GAC[CG]CT[CG]GGAG
AGCCCCAGGAGAGGCCAG[CG]C[CG][CG]CAGCAGC[CG]CCC
[CG]CTG[CG]CCCACCTCCC[CG]GCTGCTCC[CG]GAGGGCTC
ACAAAGG[CG]GTGGC[CG]CC[CG]AGTGGCCTTCTCCATCCAG
G[CG]TT[CG][CG]TCCTCCTCCCCACCTTCTCTCC[CG]AAGG
[CG]AAAATGGCAGGGCCAGG[CG]AGAACCTGGGACAG[[CG]]
GTGGCCCTAGCCCTG[[CG]]ATCCTCACCCCTCCTGCTAGGAGA
GGCTG[CG]GGCTGCC[CG][CG]GA[CG]ATGTGGC[CG][CG]
GCTGCTCC[CG]AG[CG]CATCTT[CG]GCC[CG]GGTCCC[CG]
C[CG]CCACCCCTCTTCTCTGCTCCTTCCATCC[CG]CCCA
SEQ ID NO: 5399
GGGTGCAGAAG[CG]AGACT[CG]AGAGTCCAC[CG]GCCAGGGG
[CG]TCTGTCCA[CG]GGTCTGCA[CG]GGAG[CG]CAC[CG]C[
CG]CT[CG]GCC[CG]GGGG[CG]TC[CG]TGG[CG]CTGGGAG[
CG]AGAGCTT[CG]G[CG]G[CG]GCTG[CG]GGCAGGG[CG][C
G][CG]GAG[CG]GGCTGCAGGTG[CG]GG[CG]GAGGC[CG]G[
CG]GGGGG[CG]C[CG]GGGG[CG]CTG[CG][CG]GCCCTTGG[
CG]G[CG]GGGG[CG][CG][CG]GGGTGG[CG]GGGGAGGC[CG
]AGG[CG]C[CG]GCAGCTT[CG][CG]C[CG]G[CG]GCTGGAA
G[CG]GG[CG]GGCTGCA[CG]GG[CG]GCT[CG]AGTG[CG]GG
GACCCCAGCCCCT[CG]CCCT[CG]TGAG[CG]C[CG]CCCCTGC
CACCTGCTGCCAAGTCAC[CG]GACTGGGGATG[CG]GGGGG[CG
]GGGTG[CG]GGGAGAGGAAAG[CG]C[CG][CG]GGTGGCA[CG
]GTGCCTCTGG[CG]ACAGAGTAGATGGACC[CG]AAAGAGACC[
[CG]]AGGTGACTT[CG]GGCTGGGCAGGACACCCC[CG]GGAGT
TGCCAG[[CG]]GCTCTCAG[CG]GG[CG]CCTCTAGGACCCCTT
GTCACTG[CG]AGCAG[CG]CCTGCTCTGGGGAGGGGCT[CG]AA
GG[CG]C[CG][CG]CCTTCTCAA[CG][CG]C[CG]CTG[[CG]
]TC
SEQ ID NO: 5400
ATCATTGTTGTTGGTTCCAGGAGGAG[CG]AAG[CG]CCTGGAGA
T[CG]GG[CG][CG][CG]GG[CG][CG]GCTCTGGGAG[CG]GG
GGAAGGTGAGGGGAAATGGGACATGATGGCATTAATAAT[CG]TT
ACCAAAGA[CG]GTGGGAGTAACAAAGGAATG[CG]TCTC[CG]A
GA[CG]C[CG]CCCAGCCC[CG]GGGTGTTTTCTGG[CG][CG]G
T[CG][CG]GTGCAGGAA[CG][CG]AGCCC[CG]C[CG]C[CG]
AGCCTCC[CG][CG]CCTG[CG]TT[CG]TTGG[CG][CG]G[CG
]CC[CG]G[CG]GTAGTGCCACCC[CG]AGGC[CG]AG[CG]CTG
CCCC[CG]C[CG]GCC[CG][CG]GCTGCCAGC[CG]GCCCTGCC
[CG][CG]CC[CG]GGCCC[[CG]][[CG]]AG[CG]GC[[CG]]
CACTTCACCTTA[CG]GAGGGGAGATAATGAGATCAATTAGAGG[
CG]C[CG]TCAC[CG][CG]C[CG]GAGACAGCTGC[CG]C[CG]
CATAGTAATCACC[CG][CG]GGCTGGGTG[CG][CG]GGGGCTC
CC[CG]CTACCTG[CG][CG]CCTGCTGCTCCCACCA[CG][CG]
GCAC[CG]ACC[CG]GG[CG][CG]CCCC[CG]GCCCCTGTC[CG
]CAGCCCACAGCCACAC[CG][CG]CACCCTACACCCTCCTTG[C
G]CCTCTGCTGGGGAGCTCACCCCCTCCACT[CG]CACAGTG[CG
]CTG[CG]GCC[CG]GGGTGTGGGAGGTCC[CG]GGACTTGGGGT
GTGAGTGCCTGTGTGGGGGTAGGGGCAGGT
SEQ ID NO: 5401
TCCTCCTTCAAAACCAAACCAAACCACCACCAACAAAAACTTCTG
TGCTACTCTGGCTTCTCTGGTTTTGTTTGGGAACAGTGGAACCAA
GTTAGCC[CG]GGCCAGGG[CG]CAGATGATGGACTCAGAG[CG]
CCCAGGGACCCTAGAGAGAGGAGCACTCCTCAAGAGCCCCCTGGC
CATCACC[CG]AG[CG]CCCTGGAG[CG]CCATCACC[CG]AA[C
G][CG][CG]CTCCAGGCCCT[CG]AACAAGGCCTCTGGCTGCCA
GAG[CG]AGTGAGGGG[CG]CAGAGG[CG]GCAGAGAG[CG]GAG
AGCCC[CG]GTGTCTC[[CG]][CG]AGGG[CG]G[CG]G[CG]G
CCAGCAGA[CG]G[CG]AT[CG]AGG[CG][CG][CG]CCA[CG]
GCA[CG]GCCAG[CG]CAGACA[CG]C[CG][CG]GGGTCT[CG]
GGC[CG]GAGC[CG]TGCAGC[CG]GGCC[CG]CTGCCTCTTTGC
CCCTCATGGCTC[CG][CG][CG]GGAGGAAAC[CG]GGCCTTCT
C[CG]CC[CG]CCCTCCTCT[CG]CTG[CG]GTGTCCCCAGCACC
CC[CG]CCC[CG]CCCC[CG][CG][CG]GTCCCCAC[CG]T[CG
][CG]CTCCCCTCCCTCCT[CG]CCCT[CG]CTC[CG][CG]CCT
CCTGTTGGCAGAGATGGCCT[CG]AAGGCCTCTTCCAATTTGAAT
SEQ ID NO: 5402
ACACACATGCACACATTTTCT[CG][CG]CTCTCTC[CG]GCTCT
CCTTTGTTTATTTTCTAATCTATATTTTTACTGGAAGATTTCCTC
TTTATTCTCTCC[CG]CCCTCCTACAAG[CG]CTCTTGCTGGC[C
G]TCTGGGTGCACACAC[CG]CTCCCT[CG]ATCACCCCAGCCCC
CTTCCTGGTCTCC[CG]AG[CG][CG]GGGTTTGAAGGTCACCTC
CTTTCCAGTCCC[CG]TG[CG]AGC[CG][CG]CTGC[CG]C[CG
]CCTCCTCCAGCCAGAGT[CG]GTGGGACTGGCTG[CG]CTGCCC
TGAAGTGGTTCTCCAAGCAG[[CG]][[CG]]GAGGGTGG[[CG]
]GA[CG]G[CG]GA[CG]GAGCCCAGGGGC[CG][CG]T[CG]GG
TGGGGAAACC[CG]AACT[CG][CG]GAGGGGAATCCCTCCCCCT
C[CG]CCCCAGCCCCCCAGCAGCACC[CG][CG]GTGGGG[CG]G
GGG[CG]CTCTGCCAGCCC[CG]GGAACAGCAGAGG[CG]G[CG]
GCACTGGCTGGACCCA[CG][CG][CG][CG]CCTC[CG]GGGCT
GAAGAAGGAAGGAGTGAGC[CG]AGC[CG]AGCACCCCACATCTG
GAGGGGACAGCCAGC[CG]TGGGCCC[CG]CCC[CG]G[[CG]]T
C[CG]GAGCAGGAGAACTC[CG]AGCTTCTTGCCCAG
SEQ ID NO: 5403
GTGCC[CG]GGAAC[CG]CT[CG]C[CG]AAGGAGGAAG[CG]C[
CG]TC[CG]TCTGTCCTTCCC[CG]G[CG]G[CG]G[CG]G[CG]
AG[CG]GAG[CG]CAGGAGCCC[CG]CACTCCACACACTCATGCA
CACACACACTCACACACACTCACACACTCTCCCACAACACAATAC
CCTGACACACACTCACA[CG]CA[CG]C[CG]CACTCACAC[CG]
GGTGCCTGGGAAAAT[CG]CAGA[CG]C[CG]GGGAGGAGCAGGG
GG[CG]GTGATGGGAAGGGGAGCTG[CG]AGG[CG]AAGTGTTCT
TCAGGGAAG[CG]GGCT[CG]AGTCTC[CG]CAGCTG[CG]G[CG
]G[CG]G[CG]G[CG]G[CG][CG]CTGGGC[CG]G[CG]G[CG]
GG[CG][CG]GGCAGGGGGC[CG]GGGGTGC[CG][CG][CG]GT
AGGAGCCTGGCTTGGACAGC[[CG]]AGGAGCAGGAAGTGGCCTC
TGACCCAGGACAC[[CG]]AGCAGGGCTCTCTTGCCTGGATT[CG
]CCTTTACAAAGTAACT[CG][CG]AAGGTCCC[[CG]]GAGGGG
[CG]TCAAG[CG]G[CG]ACAG[CG]GCC[CG]CC[CG]CC[CG]
CAGTC[CG]AAGCCCCCAGCCCTGCTGTCTTGCCTTCCCCCATTC
CATCAGTTGCCATTGCAA[CG]CCAATCCTGTTACA[CG]ATACA
GACT
SEQ ID NO: 5404
CCCTG[[CG]]CAA[CG]CCTCCT[CG]TCCTC[CG]AGATTTTC
CAATGGTGG[CG]CAAGCTCCTCCCT[CG]CC[CG]AGCTGGG[C
G]C[CG]TCCTAGAGCCAAG[CG]CC[CG]CTAGAGC[CG]AGAC
TAGGGGG[CG]GTGAGGAGTGGGACC[CG]CT[CG][CG]GCCAT
TGGTTGTG[CG]GCCAGGAGGGG[CG]GAGGGA[CG]A[CG][CG
][CG]GGCTGTG[CG]GGGC[CG]GCAGCC[CG]GATCCACTCC[
CG]CTG[CG]CCTGCTGTC[CG]C[CG]TG[CG]CCCAGACTG[C
G][CG]C[CG][CG]C[CG]CTG[CG]CCCAACATTCC[CG]AGG
A[CG]GCTT[CG][CG]GG[CG][CG]TAT[CG]TCCAGAC[CG]
GAGCAC[CG]CCCCAC[CG]CTAG[CG]CAGGAGACCTGC[CG]G
GGAAGT[CG][[CG]]TGTCCTGACCTGCAGGTCAGTGAGGGAAA
CTTGCAGGGTCC[CG]AGCTCTGGGGGTGAACTTGCCCTA[CG][
CG]GAGAG[CG]CCCCACTCCTTGCCTGGGG[CG]GCCT[CG]GG
TCCTGGAGG[[CG]]AGC[CG]CCTCCCAGACTCTCCCC[CG]AG
ATCTGGGCTCC[CG]GAGATGTGGCTCCTTTCTGAGAAACACCCC
CAAATCTATCCCTTTTGAGGTGTCCTTACTGTCC[CG]AGAAGCA
AT[CG]CAGC
SEQ ID NO: 5405
C[[CG]]GCACAGCAATTAACATTGGTCCAAA[CG]GTGTCAGTG
AACTTTAGCTGCAAAGAAACTACCTCATTTAAAATTTAAGCAGAC
CACAGGGAGTAGCATTGGAGGAAAGAGAGGAGATTTTCTCCTTCA
ACATTTTGAGCATACTGGAATGCCAACCCCCTAGGTAGGTGCCAG
GGCCACCACAGGAACCTGACT[CG]CAGGAGT[[CG]]TCCTC[C
G]TCTCTA[CG][CG]AAG[CG]CCCCCT[CG]AGGTGGGCAGGG
GGTGG[CG]G[CG]G[CG]A[CG]GTGG[CG]GAGC[CG]CAGAG
[CG]AGCTAGAGAG[CG]AGAG[[CG]]GTGAGACTCTG[CG]GA
[CG]TCTTCC[CG]CC[CG]C[CG][CG]CTC[CG]CCCCACCCA
GCCTC[CG]CAGCCT[CG]GGC[CG]GAT[CG]CC[CG]G[CG]G
GGAAGATG[CG]CCTCAAGAACCAGGTAGAAG[CG]CCT[CG]G[
CG]CAGGCCC[CG]CACCCC[CG]ACTCTGCC[CG]CCCTCTGTT
A[CG]GACACC[CG]CTGGGCCA[CG]TGGT[CG][CG]ACTGGC
TTCTCCCAG[CG]GCCAGCCTGGCCACCC[CG]ACTCCCAGGGAG
GGGGAGAGGGCCCTTGCAAGAGGATCCTGCCCC[CG][CG]CTGC
CCAGACCTGAGGGCTCCTGG[CG]ACC
SEQ ID NO: 5406
CTTCATGCCTCAGAAA[CG]TGGCCTACTCTGCATT[CG]GTGTG
TG[CG]GAAGCAGCAATCACAGAGGCAGCCCTAATAC[CG]GAGG
[CG]G[CG]G[CG]GCAGCAGCAGGGCCAGGTGGTAGCT[CG]GG
GCTGAGGAT[CG][CG]G[CG]GGGGCAGC[CG]CTATGGGGCCC
AAGCCCTGACACA[CG]TACCATT[CG]CTCAAGT[CG]G[CG]G
TA[CG][CG]C[CG]CCAC[CG][CG]C[CG]AGGAGGCCACCTG
GGACTTGTGGC[CG]CAGTC[CG]A[CG]AGGG[CG]ACA[CG]A
GGGCAGA[CG]GTGTAGC[CG]AAT[CG]TAGCCAGAGCTGGG[[
CG]]G[CG]G[CG]AG[CG]CC[CG]TGCACCTTCATGTGCTTA[
CG]CAG[CG]AGCTGGGGTG[CG]TGTAGCACTTGT[CG]CAGCC
C[[CG]]CACCTTGCA[CG]TGTATGGCTTGT[CG]CTAGTGTGC
A[CG]TG[CG]AATGCTTCTTA[CG]GT[CG]CTGCTGTTGG[CG
]AAG[CG]C[CG]CT[CG]CAGCCCT[CG]AACT[CG]CATCTGA
AGGGCTTCT[CG]CCTGG[CG]GAGGCAA[CG]CAGAGACATTAG
TGCTTGTGGGT[CG]TGTTCC[CG]TCAGGTGCTTGCCACCCTCC
CCCATT[CG]TCTCTCATTTTCTGGAAAAGAACTACAAAATATT
SEQ ID NO: 5407
ACAAGTGAAATAATGAGGAAG[[CG]]TGCAGAGTGAATGCCAAG
AGAAAAGGCACAAAAACCCTATTGAGAGGCCC[CG]GC[CG]CTC
CTGG[CG]TAGTCCATCACATGGCAATAGTCCTATTTAAGC[[CG
]][[CG]]C[CG]C[CG]G[CG]CCTTTCAGCACCACTAG[CG]C
TGGCCAGCACCC[CG][[CG]]CTCTTTGGG[CG]GTGCCCA[CG
]GCAGCAGAGGCTACTGTTTCAGCCTAGGTCTCAGC[CG][CG][
CG]TTCAGCCTCCTGGGCAGAGGCAGCTG[CG]GGATAC[CG][C
G]GCCAGGGAAAG[CG][[CG]]TGGAGAGC[CG]AAAGGTG[CG
]GTGGG[CG]CAGAGGG[CG]GGCTGGCTG[CG]GGG[CG]AC[[
CG]][CG][CG]C[CG]GGGCCATGC[CG][CG]CTCCTTCCTGG
TGGACT[CG]CTAGTGCTG[CG][CG]AGG[CG]GG[CG]AGAAG
AAGG[CG]CC[CG]AGGGCAGCC[CG]C[CG]C[CG]CTCTTCCC
CTA[CG]CTGTGCCCC[CG]C[CG]CA[CG][CG]CTGCA[CG]G
TCTCT[CG]CCTGG[CG]CCTGCCA[CG][CG][CG]CAAGGCTG
GGCTGCTGTG[CG]TGTGCC[CG]CTCTG[CG]TCAC[CG]CCT[
CG]CAGCTGCATGGGCCCCC[CG]GGC[CG]CC[CG][CG]CTGC
CTCTACTCAAGGCTTCCTTCCCACCCTT[CG]GCT[CG]CAGTAC
TGCCA[CG][CG]CCCCTGGGC[CG]CCAGCACTCTGCTGTGT[C
G]CC[CG]GGGT[CG]CTCAC
SEQ ID NO: 5408
GGACCAATCAC[[CG]]GCTCTGAGAAAGTT[CG]GCAGTGGCCA
CCACATCTGGTTCT[CG]TTAACTTTTCTAAGGCAG[CG]GC[[C
G]]CTGGAGCAG[[CG]]GGGCTGG[CG]GGGTAAAAGCTCCTGG
CCAGGGCTGCCTGGAGCTGCCCCTTCCACTC[[CG]]CCCCCAGG
GAGCTCC[CG]GGTCATCCTCTCATC[CG]GGCTGCCC[CG][CG
]GCCCCCAAGGAGCCCCACCCC[CG]GGACCAAATGGCC[CG]CA
AGGTTTGGGGCAG[CG]G[CG]TTGCAGGAGATGAGCTCAG[CG]
CAAAGGGAACCC[CG]CAG[CG]G[CG]AGTG[[CG]]GCTGCTG
GCCTG[CG][CG]CTGTGGCCCCAACAGGCTGGCAGGG[CG][CG
]GG[CG]GGTGG[CG]GGGTTG[CG]GTATGAGCTTTGCTCCCTG
CCCTGGGGTCC[CG]GG[CG]CTCCTGGCTCTGGAGGCCTGGCCA
T[CG]GTC[CG]AAAAGCAAAGGGACATTGT[CG]TGGTCCCCT[
CG]GC[CG]CTT[CG]CTCCACCCAGAGA[CG]CCCTGAGGAGAA
G[CG]AGCCTTTGTA[CG]AAGGCAGAGGGCTGACTCCTAAGTTC
TGTCCCCCTCC[CG]AAGGC[[CG]]GC[CG]CTAGGGAAGACTT
GGCCACCCTCTGGAAGTACCT
SEQ ID NO: 5409
GG[CG]CTGGTGCTGCTGG[[CG]]GTG[CG][CG]ACC[CG][C
G]CCTGCACA[CG]CCCATGTACTACTTCCTCTGCCACCTGGCCT
TGGTAGA[CG][CG]GGCTTCACTACTAG[CG]TGGTGC[CG]C[
CG]CTGCTGGCCAACCTG[CG][CG]GACCAG[CG]CTCTGGCTG
C[CG][CG]CAGCCACTGCA[CG]GCCCAGCTGTG[CG]CAT[CG
]CTGGCTCTGGGTTC[CG]C[CG]AATG[CG]TCCTCCTGG[CG]
GTGATGGCTCTGGAC[CG][CG][CG]GC[CG]CAGTGTGC[CG]
CC[CG]CTG[CG]CTATG[CG]GGGCT[CG]TCTCCC[CG][CG]
CCTATGT[CG]CA[CG]CTGGCCAG[CG]CCTCCTGGCTAAG[CG
]GCCTCACCAACT[CG]GTTG[CG]CAAAC[CG][CG]CTCCTGG
CTGAG[CG]GC[CG]CTGTG[CG][[CG]]CCC[CG]CCTGCTGG
ACCACTTCATCTGTGAGCTGC[CG]G[CG]TTGCTCAAGCTGGCC
TG[CG]GAGG[CG]A[CG]GAGACACTAC[CG]AGAACCAGATGT
T[[CG]][[CG]]CC[CG][CG]TGGTCATCCTGCTGCTGC[[CG
]]TTTGC[CG]TCATCCTGGCCTCCTA[CG]GTGC[CG]TGGCC[
CG]AGCTGTCTGTTGCATG[CG]GTTCAG[CG]GAGGC[[CG]]G
AGGAGGG[CG]GTGGGCA[CG]TGTGG
SEQ ID NO: 5410
CTCCAGCCTTTCTCTCTGGGCCTAG[CG]GGACTCTCCCAGGCAG
GCCCCTCCCTGCC[CG][CG]GG[CG]CACTTTCCC[CG]CCTGC
ATCTGAGG[CG]TGG[CG]CAGCTGGGAGCAAAGCACAGGTGGGC
TGGG[CG][CG]GTTTGGGGTT[CG]GG[[CG]]TGGCAGAGTGG
[CG]G[CG]GGGTCTGGGGAAGAGTTGTCTTGGGGGCAG[CG]CC
AGCCACCAGAGCC[CG]AGTCAG[CG][CG]GCCAGGACTCTGCA
GGGGCAGGTAG[CG]ACCCTTAGAGA[CG]CAA[CG]TGG[CG]C
AG[CG]GACT[CG]GCTG[CG]TGGGGCAAGGGC[CG]GGGC[CG
]GACACAGGAGCAG[CG]GGGATGC[CG]GGTG[CG]CTC[CG]A
GGGTGTGGCCCC[CG]GGCTGTG[CG]GGGCTCAGAGCTGCTTGC
TGGG[CG][[CG]]GTGCC[CG]ACAAAGGTAAGGCAGGAGTGGG
[CG]TCTTTCT[CG][CG]GGA[CG]CTGGGT[CG]GAGGGAGCT
TGAACTTGAGGTTGTTGGGGAGAGCCAGAAAGGA[CG]CCAGTGC
CACAAGGAG[CG]CCTGCCTTGGGG[CG]TGGAGC[CG]CTCCTC
CCCTCACTGGGCAGACC[CG]GCCAGCAACCAGGTAGGAGAGTGG
GGGCAAATGTGGCCATTCT
SEQ ID NO: 5411
CTGTTGACTTATGCAACTGGGGAGAGGGTTTTTTTAAC[CG]CTT
TGAGATATTGGTCCATCCCTTTTCTCTCAGAA[CG]TCCTCAAGG
GCA[CG]TAAGAGTTGGAGGTGACCTC[CG]GGAG[CG]CCTAAT
TTTGAGC[CG][CG]GAACTT[CG]GCCCACTTCTC[CG]AGGGG
ACCAGG[CG][CG][CG]CCAATA[CG]CATGTG[CG]GGCTCTG
AG[CG]CTCAGAG[CG][CG]CCTCTTCCTTCC[CG]GCTCCC[C
G][CG]GTG[CG]CACC[CG]CTGGCCACTCTG[CG]CA[CG][C
G][CG]C[CG]GGTGCCC[CG]GCCTAAGGC[CG]TTGACCT[[C
G]]GGTTCTCCC[CG]GCACAGT[CG]AATCCA[CG]CCAGGGCC
CTCAGGC[CG]GTAGCTGTCCTGCAGTC[CG]AG[CG]GAGCTG[
CG]ACCT[CG]GGCAGCTGGAAGCCTGTGGGCTGCTGCTTCTCCA
GGCAGG[CG]G[CG]GAGGGATGCAGGCAGGATGAG[CG]G[CG]
GTCCCTT[CG]C[CG]CCACC[CG]GAGAAC[CG][CG]GCTGGT
GCCCTTGAAGGCCCTGGGATGCCTGGGTTGGGTGGGTGAGTGAA[
CG]AGAACCCTGAGTGAGTGAGGGGAGGG[CG]GGTCCCCTCCTG
GCTTCAAATTCAACCCAATCAGCTC
SEQ ID NO: 5412
AAGGGGGAAGGAGGGTAGGGTAGAAGGAGGG[CG]GGGGAGAGAC
CCAT[CG]CCTCATCTTACATTTCCCCTCCCAAGCTCATTTAGTG
GGAACCTCTGCTGGGCTCCTG[[CG]][[CG]]CCTCCCTCCCCA
CCCCCTCAACTCTGGG[CG]ACCATCTCCCCCTCTTCCCTTGCTC
ACCCCCCACCCTTCCTGCTCTTC[CG]TCCCTCCCTAC[CG][CG
]TGCCTCCTCCCCTCCCCCT[CG]CCTCCCTCCCCTCCAG[CG]G
CAGTAGCTGTAGCAGCTTCAG[CG]AAGC[CG]GAGATGGGCAGA
GAG[CG][CG][CG][CG]G[[CG]]CAGCAGCTCCAGATTCACT
GCTCTCCCCTGCAGCTCCC[CG][CG]CCCC[CG]C[CG]CTGT[
CG]CTGCCT[CG]GTGTCCCCCAGCCCCAGT[CG][CG]CTCTTA
GGACAG[CG]C[CG]CCAC[CG]C[CG]CCTGGCCCTGCCTGCCT
CCTG[CG]C[CG][CG]CAGCCCT[CG][CG]AG[CG]CCC[CG]
GATGG[CG]CTTTACCCCTAGGAC[CG]GTGAGTAGG[CG]GGAG
AGTGCAGGGGGT[CG]GG[CG]TCAAAGCTTGG[CG][[CG]]GA
GAGAACATCTTTTGGTTTGGGAGGCACCCCAAACCAGCTGCTGTG
ATTCCC[CG]TCCTCCAGGCA
SEQ ID NO: 5413
T[CG]GGGGAAGCTCAGACTCCTAAACT[CG]CACTCTC[CG]TG
CTTTG[CG]C[CG]GGACCCCTGGCCACCCC[CG]G[CG]CCTAC
TATCC[CG]CCCTCCCTCCC[CG][CG][CG]CCC[CG]C[CG]C
T[CG]C[CG]GGACAGCCC[CG][CG]GGCCATGGAGCTGG[CG]
GT[CG]GGAACCTCAG[CG]AGGGCAA[CG][CG]AGCTGGC[CG
]GAGCCCCC[CG]CCC[CG]GAGCC[CG]GGC[CG]CTGTT[CG]
GCAT[CG]G[CG]TGGAGAACTT[CG]TCA[CG]CTGGTGGTGTT
[CG]GCCTGATCTT[CG][CG]CTGGGTGTGCTGGGCAACAGCCT
AGTGATCAC[CG]TGCTGG[CG][CG]CAGCAAGC[CG]GGCAAG
C[CG][CG]GAGCACCACCAACCTGTTCATCCTCAACCTGAGCAT
[CG]C[CG]ACCTGGCCTACCTGCTCTTCTGCATCCCCTTCCAGG
CCAC[CG]TGTA[CG][CG]CTGCCCACCTGGGTGCTGGG[CG]C
CTTCATCTGCAAGTTCATCCACTACTTCTTCAC[CG]TGTCCATG
CTGGTGAGCATCTTCACCCTGGC[CG][CG]ATGTC[CG]TGGAC
[CG]CTA[CG]TGGCCAT[CG]TGCACT[CG][CG]G[CG]CTCC
TCCTCCCTCAGGGTGTCC[CG]CAA[CG][CG]CTGCTGGG[CG]
TG
SEQ ID NO: 5414
CTC[CG][CG]CCCCCTCCC[CG]CCCCT[CG][CG]GCTCCCCC
ACCCT[CG][CG]TT[CG]GCAG[CG]CCAGGCTG[CG]CTCTCC
C[CG]GTGCACACC[CG]GC[CG][CG]C[CG]AGAACTAAATC[
CG][CG]GGCTGAAATTA[CG]TTTTAAAAATTAACATGTTGAAG
[[CG]]TGTGCCATTTAGTGAGAGGTGTTTTGGGCAAAGAATCAA
TTTAACTGTGACTGAC[CG]A[CG]GGCTTGACTGTATTAATTCT
GCTAC[[CG]]AAAAAAAAAAAAAAAAAAAAGCAATGAGC[CG]C
AAGCCTTGGACT[CG]CAGAGCTGC[CG]GTGCC[CG]TC[[CG]
]AGAGCCCCACCAG[CG][CG]GCTCA[CG]CCTCAGTCT[CG]C
[CG]CCCCAAGGTGGGATC[CG]A[CG]CCCAAAGGAGGG[CG]C
CCCAAGGTGGGATC[CG]AG[CG]GGC[CG]CCCTAGGAGGAGTG
AACCC[CG]CC[CG]GG[CG]CACAGCTC[CG]TG[CG]CCTGTC
CTCCCTCCTCCAG[CG]AAATCACACC[CG]CTTTGCCTCCTCTT
GCAGCAC[CG][CG][CG]TGCCTAGGTTCT[CG]GTCTG[CG]C
AGCTGGGAGTCTCCCCCACCAG[CG]C[CG][CG]GAGC[CG]CC
[CG]CTTC[CG]CTCCCTCTTTC[CG][CG]GGTTTGAGCCTCC[
CG]T
SEQ ID NO: 5415
ATACCAGGTGTTG[CG]TAATCTCTACTTGGAGATTAGCCACCC[
CG]TG[CG]CCCAGGTGAAAGATATCATTCTTC[CG]TG[CG]AT
C[CG]AAGTGC[CG]TGGAAGTTAGTGCCCTAGCCCAGTCCAGGA
GGAAGGGG[CG]T[CG]TGC[CG]G[CG]GTTTTAAGCT[CG]GA
AGGGGCAGGGGAATGAGCCCAGGGACCCCAG[CG]GGG[CG]CAG
GTAGGAGGCTGTG[[CG]]CT[CG]C[CG]GGTG[CG]CTC[CG]
GCCC[CG]ATTCCCAG[CG]CAGCCAGTAAGTGG[CG]CTGGGCC
T[CG]GGTTTGG[CG]GCC[CG]GAGGAG[CG]GGCTG[[CG]]G
ATTACCTGCAGCAG[CG]GGGAGC[[CG]]C[CG]AGCTCCTCCC
[CG]CC[CG]CCCCCACACCCCACCCCCAC[CG]CTGCACTAACC
C[CG]GCTCAGCTGGCTC[CG][CG]CACTTGCT[CG]GAGGAGC
[CG]GGGC[CG]AG[CG]GAC[CG]C[CG]GCTGCAGGCAG[CG]
AG[CG][CG]GCTGGGCTG[CG]GGGCTGCTTCCC[CG][CG]TC
CTC[CG]GGCC[CG]GGC[CG]CCCTCCTCC[CG]CACAGTG[CG
]GAGCAGGGAGGCCC[CG][CG]CCT
SEQ ID NO: 5416
CAGGTGCTCAGGCAGAAGTCAGC[CG]ACTCTGGGTT[CG]AGTC
CCAGCCCCACTGCTGGTCAGCTCCTTCTTATACCTCAGTTTTCCA
GCTGTAAAATGGGACTAAGGCCATTCTTGTAGGCCAATGGTGAGG
ATGGGAAATA[CG]CA[CG]TCATTACC[CG]ATGGGA[CG]GTG
CCAGCTCCCCT[[CG]]TCCAC[CG]A[CG]TGGGCC[CG]AGCT
AGGGTTCTGAGC[CG]GATC[CG]GGGGGAAC[CG][CG][CG]T
CTAGGGCTGGAAACC[CG]C[CG]CCACAG[CG]GGCTAGAGGT[
CG]TCCC[CG]CC[CG]CAACATATG[CG][[CG]]AAGGAAAGT
GCTA[CG]AA[CG]TCAAATGGC[CG]CCCCC[CG]C[CG]A[CG
]CCATCTGCTCTG[CG]AAGCAGAAA[CG]G[CG]GCAGCTG[CG
][CG]CCCAGTCCCTC[CG]CC[CG][CG]CCC[CG][CG][CG]
C[CG]AGTC[CG]GATC[CG]C[CG]CC[CG]G[CG]GCAC[CG]
[CG]GACAG[CG]CCAGCCC[CG]GGGTGG[CG]GG[CG][CG]G
GGCTGGGGG[CG]CTGGGAAGAGGGGTC[[CG]]CTGTAAGGCCC
TCC[CG]C[CG]CCT[CG]GGGCCAGTCTGGGTCTAGCCACTTCA
CCTGTGTGCTTGAAATGTCAGTTCTTTACCAGCCAGGCTGC[CG]
TTTG
SEQ ID NO: 5417
AG[CG]CTTTCTCTTTTCCCCCAGAAAGTCTCCAGGCAGCAGCTC
TGTGAGATCCTTAAGCAAGGTAAGGAAACTCACC[CG]CTG[CG]
CTG[CG]GCTT[CG]TGGCCAGCAGGGCTGGGACATACT[CG]CC
TCA[CG]CTTCAGCAC[CG]GGGACAGCTCCTGCCT[CG]CCAAC
TT[CG]GCAGCTCTTCT[CG]ATGTCT[CG]C[CG][CG]GGCTG
TGCCTCACAGGCTA[CG]GGAGGGGAGAGTGTCTCCCCA[CG]C[
CG]GGGTCC[CG]GCCTCTGGGTTTTAGGGAG[CG][CG]AATGG
GTCTC[CG]ACAGCAA[CG]GGAGCAGC[[CG]]GTGG[CG]CCT
CAGGCTG[CG]GTGGCAA[CG]AGCC[CG]ACTGCACTA[CG]GC
TTTG[CG]GGAC[CG][CG]CCAG[CG][CG]GAGGAGAC[CG]A
GCCCATCTCAG[CG]GGC[CG]GGC[CG]GACCCAGGTGAG[CG]
GCCCTGGCTCCC[CG]AGGAATCCA[CG]GCCCT[CG]CCTGC[C
G]AAGGCTTACTCCCTCCTGGTGC[CG]GGG[CG]CCCCCAGCTG
TCTGCTC[CG]CCTCTGACCCTG[CG]GGCTGCAG[CG]AAGGCT
G[CG][CG]CCCACC[CG][CG]GGGGAC[CG]GAA[CG]GAGGG
TAATG[CG]GG[CG]GTGTC[CG][CG]CC[CG]CCTAACAGT
SEQ ID NO: 5418
TC[CG]TTTTAAGTAAGAAGAAAATA[CG]TAATTA[CG]AAGGA
GGCTTTACCTTTGGGAAAACAAAAAGCCACTTTTGACCAATTATT
CCCACAGATACACACC[CG]GGTCCCCAACCACC[CG]CCAGGAG
GGCCCATCCTGGTGCCTGGAAGG[CG]GGGGTTCCTG[CG]ATCT
GGGGTTGGGATTT[CG]GCTCAGGC[CG]TG[CG][CG]GTGGCC
TTGGGT[CG][CG]CC[CG]CCCTGCCTGGGCCCCCAGCCCAAGG
GAC[CG]AG[CG]GGTG[CG]GG[CG]GGG[CG]GG[CG]GGG[C
G]GG[CG]GGG[CG]GGGGTGT[CG]GGGGCT[[CG]]GACT[CG
]GAGC[CG]TCCCTGCCAGTGGCTTT[CG]TGGG[CG]GCCC[CG
]CCTTGGAGCCCAGGCCAA[CG]AGC[CG][CG][CG]GGA[CG]
GAAGAGCTG[CG]GGTGGGGAGGGACAAG[CG]GG[CG]GAGAAG
GGG[[CG]]GCCCTGCTGGAGGCCCAG[CG]AGTCTGAGTGATTG
ACAAGTGAATGAGCTGGGAATGAAGACTCCTGGGGC[CG]GGAGG
GAGAGAGGGGGTTGGAGGGTGGTGAATGCCTGGGCCTCTGGGGCT
CCAGGCTGCCAGTGGCC[CG]ACCAGGCTCT[CG]GTGTCCTCCT
CCAACCTCAGTTTCC

TABLE 2
The nucleotide sequences of 28 regions within a select number
of the 418 DMRs with particular relevance to CIN3 and cancer,
particularly cervical or endometrial cancer. The 28 regions are
defined by the MethyLight probes SEQ ID NOs: 5703 to 5786. The forward
(SEQ ID NOs 5703 to 5730) and reverse (SEQ ID NOs 5731 to 5758)
MethyLight primers are also provided in the present Table 2.

| | | SEQ ID | | SEQ ID | | |
|---|---|---|---|---|---|---|
| | | NO: of | Reverse | NO: of | | SEQ ID |
| | Forward Primer | forward | Primer | reverse | Probe (5′ | NO: of |
| Gene | 5′ to 3′ | primer | 5′ to 3′ | primer | 6-FAM-3′ BHQ-1) | probe |
| DPP6_i | TTATCGTAGTGTTTG | 5703 | CCCACTCCGCGCT | 5731 | CGTGCGTCGCGCGCGTA | 5759 |
| | TTTGTGGAAGTC | | AAACTAA | | | |
| NTM_ii | CGAATTTCGGTAGTC | 5704 | CCGATCGCCGCGA | 5732 | TTTTTCGGGCGAGTATA | 5760 |
| | GTACGGTAT | | AT | | GGAAGTGGGTG | |
| LHX8_i | GAAGGAGGTTGCGCG | 5705 | CAAAATACGAAAC | 5733 | TTCGGGTTTAGGCGTCG | 5761 |
| | TTAGT | | AACGCGTACA | | TGACGG | |
| RALYL_ii | GCGTTTGAGAGCGGT | 5706 | CCTACTCGTCTAA | 5734 | AGCGGTAGTTCGCGGCG | 5762 |
| | AATATTAGTG | | ACTCACAACGAAA | | AGGTT | |
| FUT9_ii | CGTTGTTTTAAGAGC | 5707 | CGCAACAACTCCA | 5735 | ATCGAGGTAGCGATAGC | 5763 |
| | GCGATT | | AATTCACTACT | | GGCGGG | |
| LINC00403_iv | TCGCGGCGTGTTTGC | 5708 | CCCGATATCTCCG | 5736 | TTGGTCGTGTCGTGGCG | 5764 |
| | | | CGAAAA | | CG | |
| CLVS2_ii | TTTTGTGAGTTTTTC | 5709 | CGAACCGCTCGAA | 5737 | CGGTTGTTGCGCGGCGT | 5765 |
| | GGGAGTAGTC | | AAAACC | | TG | |
| KCNIP4_i | GAAGCGGGCGGGTTG | 5710 | GCTCACGAAAACG | 5738 | CGGGCGGTTCGAGTGCG | 5766 |
| | T | | AAAAACTAAAAT | | G | |
| NET01_i | ATCGTTTTTCGGTAG | 5711 | CAACATCGTAAAA | 5739 | ATTCGGCGACGGTTGAT | 5767 |
| | GTTTTGG | | CCGAACGTAAA | | CGCG | |
| CLEC14A_i | TCGGTTTTAGGTGGA | 5712 | CCCGAAACCGCCT | 5740 | TGTAGTTGGAAGGGCGC | 5768 |
| | TTGAAGTTTA | | CTAACTTA | | GCGATAG | |
| GSX1_iii | CGTAGAGGGCGGGTT | 5713 | GCGCAACACTAAC | 5741 | CGATCGCGCGTCGG | 5769 |
| | GGT | | GAATCCA | | | |
| ZNF536_ii | TGCGGCGGTAGTTGT | 5714 | GAACGACCGCACT | 5742 | CGCGGTGACGGCGTTTT | 5770 |
| | TTTC | | TCACCTT | | TAATTGA | |
| PDEIC_ii | GTCGTAGTGTAGTCG | 5715 | CGCGAATAAATCT | 5743 | TTATCGTAGTTTGAGGC | 5771 |
| | GGTTCGTT | | CCGACAAC | | GTTATCGGTTGTTTTC | |
| MDGA_I | GGGTGTTTGGGAAAA | 5716 | CGCCGCAACTACG | 5744 | CGAGGCGAAGTGTTTTT | 5772 |
| | TCGTAGA | | AAAACTC | | TAGGGAAGCG | |
| MHC_ii | GAGTTGGTGAGGTCG | 5717 | GCCCGCTACGCTA | 5745 | TTGGTTAGCGTGCGATA | 5773 |
| | TTTAGTTAGG | | TACGAAA | | TAGGCGCG | |
| NID2_i | TGTAGGAGATGAGTT | 5718 | CCACAACGCGCAA | 5746 | AATTTCGTAGCGGCGAG | 5774 |
| | TAGCGTAAAGG | | ACCA | | TGCGGT | |
| ZIC4 | TCGTTGCGTAAGTAT | 5719 | AACGACACGAAAA | 5747 | CGTTCGTCGTCGTTTAG | 5775 |
| | ATGAAGGTGTA | | CAAACGATATAAC | | TTTTGGTTACGATT | |
| RCN1_i | CGCGAAGGAAAGTGT | 5720 | CGCAACTACCGCC | 5748 | CGTTTTTCGTCGACGTT | 5776 |
| | TACGAA | | GTTTCTA | | ATTTGTTTTGCG | |
| RYR2_i | GCGTTGGGTTTCGGG | 5721 | CGACTCCCCGCTA | 5749 | TCGGAGGAGCGGGTTGC | 5777 |
| | TTT | | CTACAAATAA | | GG | |
| MSC_ii | GGTTTTCGTTTTTAA | 5722 | CTCCTCTACCGAC | 5750 | TTCGCGGCGTAGAGCGT | 5778 |
| | GAGGTCGTTT | | GAATTATCACTAA | | AGTTACGTT | |
| LINC00403_i | TGAGTCGTAAGTTTT | 5723 | AAAACGACGAAAC | 5751 | TTGTCGGTGTTCGTTCG | 5779 |
| | GGATTCGTAGA | | TAAAACGTAAACC | | AGAGTTTTATTAGCG | |
| RBFOX_i | CGTTGTCGTCGTTTT | 5724 | CCGCGCTACTTAA | 5752 | TCGGTGGGATTGGTTGC | 5780 |
| | TTTTAGTTAGA | | AAAACCACTT | | GTTGTTTT | |
| GALR1_i | GTGGAGAATTTCGTT | 5725 | ACGCGCCAACACG | 5753 | TGTTCGGTTTGATTTTC | 5781 |
| | ACGTTGGT | | ATAATC | | GCGTTGGG | |
| RNF291_ii | GGATTGTAGGATAGT | 5726 | GACCTAAAACCGT | 5754 | TTTTGGCGTGGATTCGA | 5782 |
| | TATCGGTTTGAG | | TAACCTCGAATT | | TTGTGTCG | |
| REG39_i | CGGATTCGGAGTCGT | 5727 | GCGACTCGTTAAC | 5755 | TAGTGGTTTTCGTGGGC | 5783 |
| | TTTTG | | CTAAACTCCAA | | GGTTTCGT | |
| RP4_ii | GCGTTTAGTAAGTAG | 5728 | AAACAAAAACCGA | 5756 | TTTTCGGAGCGTATTCG | 5784 |
| | TTTTGAGTTTCGTA | | AACCGAACA | | GTATTTTCGTTGTTT | |
| ELAVL4_i | CGGAGTCGTAGAGCG | 5729 | GAAACGCGACGAA | 5757 | CGAGAGCGGTGAGATTT | 5785 |
| | AGTTAGAG | | CGAAAA | | TGCGGA | |
| SERTM1_ii | TTGTAGGTTAGGATA | 5730 | CGAACGCGTATCG | 5758 | CGGTAGGTTTTTTGCGT | 5786 |
| | CGCGATTTTT | | TCCAAA | | TAGCGGTGG | |

TABLE 12

| | | | | | | | Amp- | |
|---|---|---|---|---|---|---|---|---|
| | | | | | | | licon | |
| | gene_ | Chro- | Rxn. | Rxn. | Amplicon. | Amplicon. | SEQ ID | |
| rxn_name | name | mosome | Start | Finish | Location | Length | NO | amplicon_sequence_5_to_3 |
| DPP6_i | DPP6 | 7 | 153584008 | 153584079 | chr7: | 72 | 5787 | TCAC[[CG]]TAGTGCTTGTTTGTGG |
| | | | | | 153584008- | | | AAGC[CG]AG[CG]TG[[CG]]TG |
| | | | | | 153584079 | | | [[CG]][[CG]][[CG]][[CG]][[CG]] |
| | | | | | | | | CACCCAGTCCAG[[CG]][[CG]]GA |
| | | | | | | | | GTGGG |
| NTM_ii | NTM | 11 | 131781252 | 131781354 | chr11: | 103 | 5788 | C[[CG]]GT[[CG]]C[[CG]][[CG]]GG |
| | | | | | 131781252- | | | TTCAC[CG]CTCAGTCCC[CG] |
| | | | | | 131781354 | | | [CG]CT[CG]CTC[CG]CACCCCA |
| | | | | | | | | CCCACTTCCTGTGCT[[CG]]CC |
| | | | | | | | | [[CG]]GGGGG[CG]TGTGC[[CG]]T |
| | | | | | | | | G[[CG]]GCTGC[[CG]]GAGTT |
| | | | | | | | | [[CG]] |
| LHX8_i | LHX8 | 1 | 75602107 | 75602185 | chr1: | 79 | 5789 | GAAGGAGGCTG[[CG]][[CG]]CCA |
| | | | | | 75602107- | | | GCC[CG]CC[CG][CG]G[CG]CC |
| | | | | | 75602185 | | | [[CG]]GGCTCAGG[[CG]]C[[CG]] |
| | | | | | | | | TGA[[CG]]GCTGCA[[CG]][[CG]]CT |
| | | | | | | | | GCCC[[CG]]CACTCTG |
| RALYL_ii | RALYL | 8 | 85095492 | 85095580 | chr8: | 89 | 5790 | CCTGCT[[CG]]TCTGGGCTCACAG |
| | | | | | 85095492- | | | [[CG]]AAGGCAGCCT[[CG]] |
| | | | | | 85095580 | | | [[CG]][[CG]]AGCTGC[[CG]]CTGC |
| | | | | | | | | [CG]CTGCTGC[CG]CCACTGGTG |
| | | | | | | | | TTGC[[CG]]CTCTCAGG[[CG]]C |
| FUT9_ii | FUT9 | 6 | 96463902 | 96464004 | chr6: | 103 | 5791 | [[CG]]CAGCAGCTCCAGATTCACT |
| | | | | | 96463902- | | | GCTCTCCCCTGCAGCTCCC[CG] |
| | | | | | 96464004 | | | [CG]CCCC[[CG]]C[[CG]]CTGT |
| | | | | | | | | [[CG]]CTGCCT[[CG]]GTGTCCCC |
| | | | | | | | | CAGCCCCAGT[[CG]][[CG]]CTC |
| | | | | | | | | TTAGGACAG[[CG]] |
| LINC00403_ | SOX1-OT | 13 | 112717233 | 112717317 | chr13: | 85 | 5792 | CC[[CG]]GTGTCTC[[CG]] |
| iv | | | | | 112717233- | | | [[CG]]AGGG[CG]G[CG]G[CG]G |
| | | | | | 112717317 | | | CCAGCAGA[CG]G[CG]AT[CG]A |
| | | | | | | | | GG[CG][[CG]][[CG]]CCA |
| | | | | | | | | [[CG]]GCA[[CG]]GCCAG[CG]CA |
| | | | | | | | | GACA[[CG]]C[[CG]][[CG]]G |
| CLVS2_ii | CLVS2 | 6 | 123317440 | 123317534 | chr6: | 95 | 5793 | [[CG]]GAC[[CG]]CT[[CG]]GGA |
| | | | | | 123317440- | | | GAGCCCCAGGAGAGGCCAG[[CG]] |
| | | | | | 123317534 | | | C[[CG]][[CG]]CAGCAGC[[CG]]CCC |
| | | | | | | | | [CG]CTG[CG]CCCACCTCCC |
| | | | | | | | | [CG]GCTGCTCC[[CG]]GAGGGCT |
| | | | | | | | | CACAAAG |
| KCNIP4_i | KCNIP4 | 4 | 21950535 | 21950596 | chr4: | 62 | 5794 | GAAG[[CG]]GG[[CG]]GGCTGCA |
| | | | | | 21950535- | | | [[CG]]GG[[CG]]GCT[[CG]]AGTG |
| | | | | | 21950596 | | | [[CG]]GGGACCCCAGCCCCT |
| | | | | | | | | [[CG]]CCCT[[CG]]TGAGC |
| NET01_i | NETO1 | 18 | 70534272 | 70534346 | chr18: | 75 | 5795 | AC[[CG]]TTCTC[[CG]]GCAGGTT |
| | | | | | 70534272- | | | TTGGGATC[[CG]]G[[CG]]A |
| | | | | | 70534346 | | | [[CG]]GCTGAC[[CG]][[CG]] |
| | | | | | | | | [CG]C[CG]CCCCCA[[CG]]CC |
| | | | | | | | | [[CG]]GTTCCA[[CG]]ATGCTG |
| CLEC14A_i | CLEC14A | 14 | 38724596 | 38724678 | chr14: | 83 | 5796 | T[[CG]]GTCCCAGGTGGACTGAAG |
| | | | | | 38724596- | | | TCCAGAG[CG]G[CG]CTGTGCAG |
| | | | | | 38724678 | | | CTGGAAGGG[[CG]][[CG]] |
| | | | | | | | | [[CG]]ATAGCTCAAGTTAGAGG |
| | | | | | | | | [[CG]]GCCC[[CG]]GG |
| GSX1_iii | GSX1 | 13 | 28366785 | 28366866 | chr13: | 82 | 5797 | [[CG]]CAGAGGG[[CG]]GGCTGGCT |
| | | | | | 28366785- | | | G[CG]GGG[[CG]]AC[[CG]] |
| | | | | | 28366866 | | | [[CG]][[CG]]C[[CG]]GGGCCATGC |
| | | | | | | | | [CG][CG]CTCCTTCCTGGTGGAC |
| | | | | | | | | T[[CG]]CTAGTGCTG[[CG]]C |
| ZNF536_ii | ZNF536 | 19 | 30866195 | 30866279 | chr19: | 85 | 5798 | GAG[[CG]]GC[[CG]]CACTTCACC |
| | | | | | 30866195- | | | TTA[CG]GAGGGGAGATAATGAGA |
| | | | | | 30866279 | | | TCAATTAGAGG[[CG]]C[[CG]]TC |
| | | | | | | | | AC[[CG]][[CG]]C[CG]GAGACAG |
| | | | | | | | | CTGC[[CG]][[CG]]CA |
| PDEIC_ii | PDE1C | 7 | 32467624 | 32467704 | chr7: | 81 | 5799 | [[CG]][[CG]]AATGGGTCTC |
| | | | | | 32467624- | | | [[CG]]ACAGCAA[CG]GGAGCAGC |
| | | | | | 32467704 | | | [[CG]]GTGG[[CG]]CCTCAGGCTG |
| | | | | | | | | [[CG]]GTGGCAA[[CG]]AGCC |
| | | | | | | | | [[CG]]ACTGCACTA[[CG]]GC |
| MDGA_I | MDGA2 | 14 | 48144084 | 48144198 | chr14: | 115 | 5800 | GGGTGCCTGGGAAAAT[[CG]]CAG |
| | | | | | 48144084- | | | A[CG]C[CG]GGGAGGAGCAGGGG |
| | | | | | 48144198 | | | G[CG]GTGATGGGAAGGGGAGCTG |
| | | | | | | | | [[CG]]AGG[[CG]]AAGTGTTCTT |
| | | | | | | | | CAGGGAAG[[CG]]GGCT[CG]AG |
| | | | | | | | | TCTC[[CG]]CAGCTG[[CG]]G |
| | | | | | | | | [[CG]] |
| MHC_ii | N/A | 6 | 29521375 | 29521459 | chr6: | 85 | 5801 | GCC[[CG]]CTG[[CG]]CTATG[[CG]] |
| | | | | | 29521375- | | | GGGCT[CG]TCTCCC[[CG]][[CG]]C |
| | | | | | 29521459 | | | CTATGT[[CG]]CA[[CG]]CTGGCCA |
| | | | | | | | | G[CG]CCTCCTGGCTAAG[[CG]]G |
| | | | | | | | | CCTCACCAACTC |
| NID2_i | NID2 | 14 | 52536129 | 52536198 | chr14: | 70 | 5802 | TGCAGGAGATGAGCTCAG[[CG]]C |
| | | | | | 52536129- | | | AAAGGGAACCC[[CG]]CAG[[CG]] |
| | | | | | 52536198 | | | G[[CG]]AGTG[[CG]]GCTGCTGGCCT |
| | | | | | | | | G[[CG]][[CG]]CTGTGG |
| ZIC4 | ZIC4 | 3 | 147108796 | 147108884 | chr3: | 89 | 5803 | GG[[CG]]ACA[[CG]]AGGGCAGA |
| | | | | | 147108796- | | | [[CG]]GTGTAGC[CG]AAT[[CG]] |
| | | | | | 147108884 | | | TAGCCAGAGCTGGG[[CG]]G |
| | | | | | | | | [[CG]]G[[CG]]AG[[CG]]CC[CG]T |
| | | | | | | | | GCACCTTCATGTGCTTA[[CG]]CA |
| | | | | | | | | G[[CG]]A |
| RCN1_i | RCN1 | 11 | 32008959 | 32009042 | chr11: | 84 | 5804 | [[CG]][[CG]]AAGGAAAGTGCT |
| | | | | | 32008959- | | | A[[CG]]AA[CG]TCAAATGGC |
| | | | | | 32009042 | | | [[CG]]CCCCC[[CG]][[CG]]A |
| | | | | | | | | [[CG]]CCATCTGCTCTG[[CG]]AAG |
| | | | | | | | | CAGAAA[[CG]]G[[CG]]GCAGCT |
| | | | | | | | | G[[CG]] |
| RYR2_i | RYR2 | 1 | 237205344 | 237205410 | chr1: | 67 | 5805 | G[[CG]]CTGGGCCT[[CG]]GGTTTGG |
| | | | | | 237205344- | | | [CG]GCC[[CG]]GAGGAG |
| | | | | | 237205410 | | | [[CG]]GGCTG[[CG]]GATTACCT |
| | | | | | | | | GCAGCAG[[CG]]GGGAGC[[CG]] |
| MSC_ii | MSC | 8 | 72756267 | 72756348 | chr8: | 82 | 5806 | CTCCTCTGC[[CG]]A[[CG]]AGTTG |
| | | | | | 72756267- | | | TCACTGGG[CG]AGG[[CG]]TAGC |
| | | | | | 72756348 | | | TG[[CG]]CTCTA[[CG]]C[[CG]] |
| | | | | | | | | [[CG]]GAGGGG[[CG]]GCCTCTT |
| | | | | | | | | GGAGG[[CG]]GGGACC |
| LINC00403_i | SOX1-OT | 13 | 112711967 | 112712052 | chr13: | 86 | 5807 | TGAGC[[CG]]CAAGCCTTGGACT |
| | | | | | 112711967- | | | [[CG]]CAGAGCTGC[[CG]]GTGCC |
| | | | | | 112712052 | | | [[CG]]TC[[CG]]AGAGCCCCACCAG |
| | | | | | | | | [[CG]][CG]GCTCA[[CG]]CCTCAG |
| | | | | | | | | TCT[[CG]][[CG]]CCCC |
| RBFOX_i | RBFOX1 | 16 | 6069328 | 6069403 | chr16: | 76 | 5808 | [[CG]]CTGC[[CG]]C[[CG]]CCTCCT |
| | | | | | 6069328- | | | CCAGCCAGAGT[[CG]]GTGGGACT |
| | | | | | 6069403 | | | GGCTG[[CG]]CTGCCCTGAAGTGGT |
| | | | | | | | | TCTCCAAGCAG[[CG]][[CG]]G |
| GALR1_i | GALR1 | 18 | 74962595 | 74962684 | chr18: | 90 | 5809 | GTGGAGAACTT[[CG]]TCA[[CG]]CT |
| | | | | | 74962595- | | | GGTGGTGTT[[CG]]GCCTGATCT |
| | | | | | 74962684 | | | T[[CG]][[CG]]CTGGGTGTGCTGG |
| | | | | | | | | GCAACAGCCTAGTGATCAC[[CG]] |
| | | | | | | | | TGCTGG[[CG]][[CG]]C |
| RNF291_ii | RNF219- | 13 | 79170284 | 79170367 | chr13: | 84 | 5810 | GGCCTAAGGC[[CG]]TTGACCT |
| | AS1 | | | | 79170284- | | | [[CG]]GGTTCTCCC[[CG]]GCACAG |
| | | | | | 79170367 | | | T[[CG]]AATCCA[[CG]]CCAGGGC |
| | | | | | | | | CCTCAGGC[[CG]]GTAGCTGTCCT |
| | | | | | | | | GCAGTCC |
| REG39_i | N/A | 19 | 22444593 | 22444663 | chr19: | 71 | 5811 | [[CG]]GACT[[CG]]GAGC[[CG]]TCC |
| | | | | | 22444593- | | | CTGCCAGTGGCTTT[[CG]]TGGG |
| | | | | | 22444663 | | | [[CG]]GCCC[[CG]]CCTTGGAGCCC |
| | | | | | | | | AGGCCAA[[CG]]AGC[[CG]]C |
| RP4_ii | N/A | 12 | 126676038 | 126676139 | chr12: | 102 | 5812 | GGGCAAGGGC[[CG]]GGGC |
| | | | | | 126676038- | | | [[CG]]GACACAGGAGCAG[[CG]] |
| | | | | | 126676139 | | | GGGATGC[[CG]]GGTG[[CG]]CT |
| | | | | | | | | C[[CG]]AGGGTGTGGCCCC[CG]G |
| | | | | | | | | GCTGTG[[CG]]GGGCTCAGAGCTG |
| | | | | | | | | CTTGCTGGG[[CG]]C |
| ELAVL4_i | ELAVL4 | 1 | 50513718 | 50513788 | chr1: | 71 | 5813 | [[CG]]GAGC[[CG]]CAGAG |
| | | | | | 50513718- | | | [[CG]]AGCTAGAGAG[[CG]]AGA |
| | | | | | 50513788 | | | G[[CG]]GTGAGACTCTG[[CG]]GA |
| | | | | | | | | [CG]TCTTCC[[CG]]CC[[CG]] |
| | | | | | | | | C[[CG]][[CG]]CTCC |
| SERTM1_ii | SERTM1 | 13 | 37248238 | 37248321 | chr13: | 84 | 5814 | [[CG]]GG[[CG]][[CG]]TAT |
| | | | | | 37248238- | | | [[CG]]TCCAGAC[CG]GAGCAC |
| | | | | | 37248321 | | | [CG]CCCCAC[[CG]]CTAG |
| | | | | | | | | [[CG]]CAGGAGACCTGC[[CG]]G |
| | | | | | | | | GGAAGT[[CG]][[CG]]TGTCCTGA |
| | | | | | | | | CCTGCAG |

Cancer-related CpGs for analysis

In any of the assays described herein, in a sample which has been taken from an individual, the sample comprises a population of DNA molecules.

The assay of the invention further comprises determining in the population of DNA molecules in the sample the methylation status of a panel of:
(i) one or more CpGs selected from a panel of CpGs identified in SEQ ID NOs 1 to 5000 wherein the CpGs are identified at nucleotide positions 61 to 62; and/or
(ii) one or more CpGs selected from within a panel of one or more Differentially Methylated Regions (DMRs) defined by SEQ ID NOs 5001 to 5418, wherein the CpGs are denoted by CG

A cancer index value is then derived based on the methylation status of the one or more CpGs in the panel, which is used to assess the presence, absence or development of CIN3 and/or cancer, particularly cervical or endometrial cancer, most preferably cervical cancer, in the individual based on the cancer index value.

In any of the assays described herein, in DNA derived from cells in the sample the methylation status of each CpG in a panel of
(i) one or more CpGs selected from a panel of CpGs identified in SEQ ID NOs 1 to 5000 wherein the CpGs are identified at nucleotide positions 61 to 62; and/or
(ii) one or more CpGs selected from within a panel of one or more Differentially Methylated Regions (DMRs) defined by SEQ ID NOs 5001 to 5418, wherein the CpGs are denoted by CG, is determined.

In any of the assays described herein, in DNA derived from cells in the sample the methylation status of each CpG in a panel of one or more CpGs from a panel of CpGs identified at nucleotide positions 61 to 62 in SEQ ID NOs 1 to 5000, is determined.

In any of the assays described herein, the panel of one or more CpGs may comprise at least 500 CpGs selected from the CpGs identified at nucleotide positions 61 to 62 in SEQ ID NOs 1 to 5000, preferably wherein the assay is characterised as having a receiver operating characteristics (ROC) area under the curve (AUC) of at least 0.80. The panel of one or more CpGs may comprise at least the CpGs identified at nucleotide positions 61 to 62 in SEQ ID NOs 1 to 5000, preferably wherein the assay is characterised as having an ROC AUC of at least 0.92.

In any of the assays described herein, the panel of one or more CpGs may comprise at least 1000 CpGs selected from the CpGs identified at nucleotide positions 61 to 62 in SEQ ID NOs 1 to 5000, preferably wherein the assay is characterised as having a ROC AUC of at least 0.80. The panel of one or more CpGs may comprise at least the CpGs identified in SEQ ID NOs 1 to 1000 and identified at nucleotide positions 61 to 62, preferably wherein the assay is characterised as having a ROC AUC of at least 0.92.

In any of the assays described herein, the panel of one or more CpGs may comprise at least 1500 CpGs selected from the CpGs identified at nucleotide positions 61 to 62 in SEQ ID NOs 1 to 5000, preferably wherein the assay is characterised as having a ROC AUC of at least 0.82. The panel of one or more CpGs may comprise at least the CpGs identified in SEQ ID NOs 1 to 1500 and identified at nucleotide positions 61 to 62, preferably wherein the assay is characterised as having a ROC AUC of at least 0.92.

In any of the assays described herein, the panel of one or more CpGs may comprise at least 2000 CpGs selected from the CpGs identified at nucleotide positions 61 to 62 in SEQ ID NOs 1 to 5000, preferably wherein the assay is characterised as having an AUC of at least 0.81. The panel of one or more CpGs may comprise at least the CpGs identified in SEQ ID NOs 1 to 200 and identified at nucleotide positions 61 to 62, preferably wherein the assay is characterised as having a ROC AUC of at least 0.92.

In any of the assays described herein, the panel of one or more CpGs may comprise at least the 5000 CpGs identified at nucleotide positions 61 to 62 in SEQ ID NOs 1 to 5000, and further wherein the assay is characterised as having a ROC AUC of at least 0.92.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the assays described herein, the panel of one or more CpGs may comprise at least 500 CpGs selected from the CpGs identified at nucleotide positions 61 to 62 in SEQ ID NOs 1 to 5000, optionally wherein:
1. the assay is characterised as having an ROC AUC (AUC) of at least 0.92, and more preferably wherein the panel of one or more CpGs comprises at least the CpGs identified in SEQ ID NOs 1 to 500 and identified at nucleotide positions 61 to 62;
2. the assay is characterised as having an AUC of at least 0.90, and more preferably wherein the panel of one or more CpGs comprises at least the CpGs identified in SEQ ID NOs 501 to 1000 and identified at nucleotide positions 61 to 62;
3. the assay is characterised as having an AUC of at least 0.88, and more preferably wherein the panel of one or more CpGs comprises at least the CpGs identified in SEQ ID NOs 1001 to 1500 and identified at nucleotide positions 61 to 62;
4. the assay is characterised as having an AUC of at least 0.86, and more preferably wherein the panel of one or more CpGs comprises at least the CpGs identified in SEQ ID NOs 1501 to 2000 and identified at nucleotide positions 61 to 62;
5. the assay is characterised as having an AUC of at least 0.86, and more preferably wherein the panel of one or more CpGs comprises at least the CpGs identified in SEQ ID NOs 2001 to 2500 and identified at nucleotide positions 61 to 62;
6. the assay is characterised as having an AUC of at least 0.83, and more preferably wherein the panel of one or more CpGs comprises at least the CpGs identified in SEQ ID NOs 2501 to 3000 and identified at nucleotide positions 61 to 62;
7. the assay is characterised as having an AUC of at least 0.82, and more preferably wherein the panel of one or more CpGs comprises at least the CpGs identified in SEQ ID NOs 3001 to 3500 and identified at nucleotide positions 61 to 62;
8. the assay is characterised as having an AUC of at least 0.82, and more preferably wherein the panel of one or more CpGs comprises at least the CpGs identified in SEQ ID NOs 3501 to 4000 and identified at nucleotide positions 61 to 62; or
9. the assay is characterised as having an AUC of at least 0.80, and more preferably wherein the panel of one or more CpGs comprises at least the CpGs identified in SEQ ID NOs 4001 to 4500 and identified at nucleotide positions 61 to 62;
10. the assay is characterised as having an AUC of at least 0.81, and more preferably wherein the panel of one or more CpGs comprises at least the CpGs identified in SEQ ID NOs 4501 to 5000 and identified at nucleotide positions 61 to 62.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 100 CpGs selected from the CpGs identified at nucleotide positions 61 to 62 in SEQ ID NOs 1 to 5000, preferably wherein the assay is characterised as having an AUC of at least 0.90, and more preferably wherein the panel of one or more CpGs comprises at least the CpGs identified in SEQ ID NOs 1 to 100 and identified at nucleotide positions 61 to 62;
2. at least 500 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 500 and identified at nucleotide positions 61 to 62;
3. at least 1000 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 1000 and identified at nucleotide positions 61 to 62;
4. at least 1500 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 1500 and identified at nucleotide positions 61 to 62;
5. at least 2000 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 2000 and identified at nucleotide positions 61 to 62;
6. at least 2500 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 2500 and identified at nucleotide positions 61 to 62;
7. at least 3000 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 3000 and identified at nucleotide positions 61 to 62;
8. at least 3500 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 3500 and identified at nucleotide positions 61 to 62;
9. at least 4000 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 4000 and identified at nucleotide positions 61 to 62;
10. at least 4500 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified in SEQ ID NOs 1 to 4500 and identified at nucleotide positions 61 to 62; or
11. at least 5000 CpGs selected from the CpGs identified in SEQ ID NOs 1 to 500 and identified at nucleotide positions 61 to 62, preferably wherein the assay is characterised as having an AUC of at least 0.92, and more preferably wherein the panel of one or more CpGs comprises at least the CpGs identified at nucleotide positions 61 to 62 in SEQ ID NOs 1 to 5000.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the panel of one or more CpGs may comprise:
1. at least 500 CpGs selected from the CpGs identified at nucleotide positions 61 to 62 in SEQ ID NOs 1 to 5000, preferably wherein the assay is characterised as having an AUC of at least 0.81, and more preferably wherein the panel of one or more CpGs comprises at least the CpGs identified in SEQ ID NOs 4501 to 5000 and identified at nucleotide positions 61 to 62;
2. at least 1000 CpGs selected from the CpGs identified at nucleotide positions 61 to 62 in SEQ ID NOs 1 to 5000, preferably wherein the assay is characterised as having an AUC of at least 0.80, and more preferably wherein the panel of one or more CpGs comprises at least the CpGs identified in SEQ ID NOs 4001 to 5000 and identified at nucleotide positions 61 to 62;
3. at least 1500 CpGs selected from the CpGs identified at nucleotide positions 61 to 62 in SEQ ID NOs 1 to 5000, preferably wherein the assay is characterised as having an AUC of at least 0.82, and more preferably wherein the panel of one or more CpGs comprises at least the CpGs identified in SEQ ID NOs 3501 to 5000 and identified at nucleotide positions 61 to 62;
4. at least 2000 CpGs selected from the CpGs identified at nucleotide positions 61 to 62 in SEQ ID NOs 1 to 5000, preferably wherein the assay is characterised as having an AUC of at least 0.81, and more preferably wherein the panel of one or more CpGs comprises at least the CpGs identified in SEQ ID NOs 3001 to 5000 and identified at nucleotide positions 61 to 62;
5. at least 2500 CpGs selected from the CpGs identified at nucleotide positions 61 to 62 in SEQ ID NOs 1 to 5000, preferably wherein the assay is characterised as having an AUC of at least 0.82, and more preferably wherein the panel of one or more CpGs comprises at least the CpGs identified in SEQ ID NOs 2501 to 5000 and identified at nucleotide positions 61 to 62;
6. at least 3000 CpGs selected from the CpGs identified at nucleotide positions 61 to 62 in SEQ ID NOs 1 to 5000, preferably wherein the assay is characterised as having an AUC of at least 0.82, and more preferably wherein the panel of one or more CpGs comprises at least the CpGs identified in SEQ ID NOs 2001 to 5000 and identified at nucleotide positions 61 to 62;
7. at least 3500 CpGs selected from the CpGs identified at nucleotide positions 61 to 62 in SEQ ID NOs 1 to 5000, preferably wherein the assay is characterised as having an AUC of at least 0.82, and more preferably wherein the panel of one or more CpGs comprises at least the CpGs identified in SEQ ID NOs 1501 to 5000 and identified at nucleotide positions 61 to 62;
8. at least 4000 CpGs selected from the CpGs identified at nucleotide positions 61 to 62 in SEQ ID NOs 1 to 5000, preferably wherein the assay is characterised as having an AUC of at least 0.84, and more preferably wherein the panel of one or more CpGs comprises at least the CpGs identified in SEQ ID NOs 1001 to 5000 and identified at nucleotide positions 61 to 62;
9. at least 4500 CpGs selected from the CpGs identified at nucleotide positions 61 to 62 in SEQ ID NOs 1 to 5000, preferably wherein the assay is characterised as having an AUC of at least 0.88, and more preferably wherein the panel of one or more CpGs comprises at least the CpGs identified in SEQ ID NOs 501 to 5000 and identified at nucleotide positions 61 to 62;
10. at least 4900 CpGs selected from the CpGs identified at nucleotide positions 61 to 62 in SEQ ID NOs 1 to 5000, preferably wherein the assay is characterised as having an AUC of at least 0.91, and more preferably wherein the panel of one or more CpGs comprises at least the CpGs identified in SEQ ID NOs 101 to 5000 and identified at nucleotide positions 61 to 62; or
11. at least 5000 CpGs selected from the CpGs identified at nucleotide positions 61 to 62 in SEQ ID NOs 1 to 5000, preferably wherein the assay is characterised as having an AUC of at least 0.92, and more preferably wherein the panel of one or more CpGs comprises at least the CpGs identified at nucleotide positions 61 to 62 in SEQ ID NOs 1 to 5000.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the above-described assays, the assay may be characterised as having a ROC AUC of 0.60 or more, 0.61 or more, 0.62 or more, 0.63 or more, 0.64 or more, 0.65 or more, 0.66 or more, 0.67 or more, 0.68 or more, 0.69 or more, 0.70 or more, 0.71 or more, 0.72 or more, 0.73 or more, 0.74 or more, 0.75 or more, 0.76 or more, 0.77 or more, 0.78 or more, 0.79 or more, 0.80 or more, 0.81 or more, 0.82 or more, 0.83 or more, 0.84 or more, 0.85 or more, 0.86 or more, 0.87 or more, 0.88 or more, 0.89 or more or 0.90 or more.

In any of the assays described herein, the step of determining the methylation status of the one or more CpGs in the panel may comprise determining the methylation status of one or more CpGs selected from within a panel of one or more Differentially Methylated Regions (DMRs) defined by SEQ ID NOs 5001 to 5418, wherein selected CpGs in each DMR are denoted by CG. The nucleotide sequences of the 418 DMRs are defined respectively by the nucleotide sequences of SEQ ID NO: 5001 to 5418 as set out in Table 1, accepting that variation in the nucleotide sequence of any given DMR may exist due to sequencing errors and/or variation between individuals. In each sequence shown below the cytosine of the CG dinucleotide motifs identified in square brackets and double square brackets is a cytosine of a CpG which may be included in a panel of CpGs when performing the assays of the invention.

In any of the assays described herein, the step of determining the methylation status of the one or more CpGs in the panel may comprise determining the methylation status of one or more CpGs denoted by CG within any one or more DMRs or within any combination of two or more DMRs defined by SEQ ID NOs 5001 to 5418, wherein selected CpGs in each DMR are denoted by CG. The DMRs are selected from the group consisting of DMRs 1 to 418 (SEQ ID NOs 5001 to 5418; as set out in Table 1).

The step of determining the methylation status of a panel of one or more CpGs may comprise determining a cancer index value of one or more of the CpGs denoted by CG within any one of the DMRs 1 to 418, or within any combination of two or more DMRs of 1 to 418.

The step of determining the methylation status of a panel of one or more CpGs may comprise determining a cancer index value of two or more, three or more, four or more, five or more, six or more, seven or more, eight or more, or nine or more of the CpGs denoted by CG within any one of the DMRs 1 to 418 (defined by SEQ ID NOs 5001 to 5418), optionally within any combination of two or more DMRs of 1 to 418.

The panel of one or more CpGs may comprise two or more CpGs of the DMR(s), three or more CpGs of the DMR(s), four or more CpGs of the DMR(s) or all CpGs of the DMR(s).

The step of determining the methylation status of a panel of one or more CpGs may comprise determining a cancer index value of least two or more, three or more, four or more, five or more, six or more, seven or more, eight or more, or nine or more of the CpGs denoted by CG within any one of the DMRs 1 to 418, or within:
a. any combination of two, three, four, five, six, seven, eight, or nine or more of DMRs 1 to 418;
b. any combination of ten, twenty, thirty, forty, fifty, sixty, seventy, eighty, or ninety or more of DMRs 1 to 418;
c. all 418 of DMRs 1 to 418;
d. one DMR defined by SEQ ID NO: 5001, two DMRs defined by SEQ ID NOs: 5001 to 5002, three DMRs defined by SEQ ID NOs: 5001 to 5003, four DMRs defined by SEQ ID NOs: 5001 to 5004, five DMRs defined by SEQ ID NOs: 5001 to 5005, six DMRs defined by SEQ ID NOs: 5001 to 5006, seven DMRs defined by SEQ ID NOs: 5001 to 5007, eight DMRs defined by SEQ ID NOs: 5001 to 5008, or nine DMRs defined by SEQ ID NOs: 5001 to 5009;
e. any combination of one or more DMRs defined by SEQ ID NO: 5391, SEQ ID NO: 5392, SEQ ID NO: 5393, SEQ ID NO: 5407 and SEQ ID NO: 5414, preferably within all of SEQ ID NO: 5391, SEQ ID NO: 5392, SEQ ID NO: 5393, SEQ ID NO: 5407 and SEQ ID NO: 5414;
f. any combination of one or more DMRs defined by SEQ ID NO: 5392, SEQ ID NO: 5393 and SEQ ID NO: 5407, preferably within all of SEQ ID NO: 5392, SEQ ID NO: 5393 and SEQ ID NO: 5407; or
g. ten DMRs defined by SEQ ID NOs: 5001 to 5010, twenty DMRs defined by SEQ ID NOs: 5001 to 5020, thirty DMRs defined by SEQ ID NOs: 5001 to 5030, forty DMRs defined by SEQ ID NOs: 5001 to 5040, fifty DMRs defined by SEQ ID NOs: 5001 to 5050, sixty DMRs defined by SEQ ID NOs: 5001 to 5060, seventy DMRs defined by SEQ ID NOs: 5001 to 5070, eighty DMRs defined by SEQ ID NOs: 5001 to 5080, or ninety DMRs defined by SEQ ID NOs: 5001 to 5090;
h. fifty DMRs defined by SEQ ID NOs: 5001 to 5050, SEQ ID NOs: 5051 to 5100, SEQ ID NOs: 5101 to 5150, SEQ ID NOs: 5151 to 5200, SEQ ID NOs: 5201 to 5250, SEQ ID NOs: 5301 to 5350, or SEQ ID NOs: 5341 to 5418; or
i. eighty one DMRs defined by SEQ ID NOs: 5015, 5016, 5017, 5025, 5026, 5027, 5028, 5029, 5032, 5033, 5048, 5049, 5050, 5053, 5054, 5057, 5068, 5069, 5071, 5072, 5073, 5074, 5075, 5076, 5077, 5083, 5090, 5091, 5092, 5093, 5094, 5095, 5096, 5099, 5102, 5137, 5138, 5140, 5143, 5146, 5147, 5148, 5149, 5150, 5151, 5164, 5165, 5167, 5175, 5176, 5177, 5179, 5180, 5185, 5204, 5224, 5226, 5228, 5232, 5246, 5248, 5285, 5287, 5293, 5307, 5309, 5315, 5317, 5324, 5328, 5337, 5339, 5340, 5348, 5349, 5354, 5361, 5362, 5366, 5368 and 5377.

In any of the assays described herein, the step of determining the methylation status of a panel of one or more CpGs may comprise determining the methylation status of one or more CpGs within any one or more DMRs selected from the group of DMRs consisting of DMRs 1 to 418 as defined by SEQ ID NOs 5001 to 5418, including:
1. one or more CpGs within DMR 1 as defined by SEQ ID NO: 5001 denoted by CG, preferably wherein the assay is characterised as having an ROC AUC of at least 0.894, and more preferably wherein the panel of one or more CpGs comprises at least the CpGs denoted by [[CG]];
2. one or more CpGs within DMR 2 as defined by SEQ ID NO: 5002 denoted by CG, preferably wherein the assay is characterised as having an ROC AUC of at least 0.894, and more preferably wherein the panel of one or more CpGs comprises at least the CpGs denoted by [[CG]];
3. one or more CpGs within DMR 3 as defined by SEQ ID NO: 5003 denoted by CG, preferably wherein the assay is characterised as having an ROC AUC of at least 0.894, and more preferably wherein the panel of one or more CpGs comprises at least the CpGs denoted by [[CG]];
4. one or more CpGs within DMR 4 as defined by SEQ ID NO: 5004 denoted by CG, preferably wherein the assay is characterised as having an ROC AUC of at least 0.894, and more preferably wherein the panel of one or more CpGs comprises at least the CpGs denoted by [[CG]];
5. one or more CpGs within DMR 5 as defined by SEQ ID NO: 5005 denoted by CG, preferably wherein the assay is characterised as having an ROC AUC of at least 0.894, and more preferably wherein the panel of one or more CpGs comprises at least the CpGs denoted by [[CG]];
6. one or more CpGs within DMR 6 as defined by SEQ ID NO: 5006 denoted by CG, preferably wherein the assay is characterised as having an ROC AUC of at least 0.876, and more preferably wherein the panel of one or more CpGs comprises at least the CpGs denoted by [[CG]];
7. one or more CpGs within DMR 7 as defined by SEQ ID NO: 5007 denoted by CG, preferably wherein the assay is characterised as having an ROC AUC of at least 0.874, and more preferably wherein the panel of one or more CpGs comprises at least the CpGs denoted by [[CG]];
8. one or more CpGs within DMR 8 as defined by SEQ ID NO: 5008 denoted by CG, preferably wherein the assay is characterised as having an ROC AUC of at least 0.874, and more preferably wherein the panel of one or more CpGs comprises at least the CpGs denoted by [[CG]];
9. one or more CpGs within DMR 9 as defined by SEQ ID NO: 5009 denoted by CG, preferably wherein the assay is characterised as having an ROC AUC of at least 0.874, and more preferably wherein the panel of one or more CpGs comprises at least the CpGs denoted by [[CG]];
10. one or more CpGs within DMR 10 as defined by SEQ ID NO: 5010 denoted by CG, preferably wherein the assay is characterised as having an ROC AUC of at least 0.874, and more preferably wherein the panel of one or more CpGs comprises at least the CpGs denoted by [[CG]];
11. one or more CpGs within DMR 11 as defined by SEQ ID NO: 5011 denoted by CG, preferably wherein the assay is characterised as having an ROC AUC of at least 0.874, and more preferably wherein the panel of one or more CpGs comprises at least the CpGs denoted by [[CG]];
12. one or more CpGs within DMR 12 as defined by SEQ ID NO: 5012 denoted by CG, preferably wherein the assay is characterised as having an ROC AUC of at least 0.874, and more preferably wherein the panel of one or more CpGs comprises at least the CpGs denoted by [[CG]];
13. one or more CpGs within DMR 13 as defined by SEQ ID NO: 5013 denoted by CG, preferably wherein the assay is characterised as having an ROC AUC of at least 0.874, and more preferably wherein the panel of one or more CpGs comprises at least the CpGs denoted by [[CG]];
14. one or more CpGs within DMR 14 as defined by SEQ ID NO: 5014 denoted by CG, preferably wherein the assay is characterised as having an ROC AUC of at least 0.87, and more preferably wherein the panel of one or more CpGs comprises at least the CpGs denoted by [[CG]];
15. one or more CpGs within DMR 15 as defined by SEQ ID NO: 5015 denoted by CG, preferably wherein the assay is characterised as having an ROC AUC of at least 0.869, and more preferably wherein the panel of one or more CpGs comprises at least the CpGs denoted by [[CG]];
16. one or more CpGs within DMR 16 as defined by SEQ ID NO: 5016 denoted by CG, preferably wherein the assay is characterised as having an ROC AUC of at least 0.869, and more preferably wherein the panel of one or more CpGs comprises at least the CpGs denoted by [[CG]];
17. one or more CpGs within DMR 17 as defined by SEQ ID NO: 5017 denoted by CG, preferably wherein the assay is characterised as having an ROC AUC of at least 0.869, and more preferably wherein the panel of one or more CpGs comprises at least the CpGs denoted by [[CG]];
18. one or more CpGs within DMR 18 as defined by SEQ ID NO: 5018 denoted by CG, preferably wherein the assay is characterised as having an ROC AUC of at least 0.868, and more preferably wherein the panel of one or more CpGs comprises at least the CpGs denoted by [[CG]];
19. one or more CpGs within DMR 19 as defined by SEQ ID NO: 5019 denoted by CG, preferably wherein the assay is characterised as having an ROC AUC of at least 0.868, and more preferably wherein the panel of one or more CpGs comprises at least the CpGs denoted by [[CG]];
20. one or more CpGs within DMR 20 as defined by SEQ ID NO: 5020 denoted by CG, preferably wherein the assay is characterised as having an ROC AUC of at least 0.866, and more preferably wherein the panel of one or more CpGs comprises at least the CpGs denoted by [[CG]];
21. one or more CpGs within DMR 21 as defined by SEQ ID NO: 5021 denoted by CG, preferably wherein the assay is characterised as having an ROC AUC of at least 0.863, and more preferably wherein the panel of one or more CpGs comprises at least the CpGs denoted by [[CG]];
22. one or more CpGs within DMR 22 as defined by SEQ ID NO: 5022 denoted by CG, preferably wherein the assay is characterised as having an ROC AUC of at least 0.863, and more preferably wherein the panel of one or more CpGs comprises at least the CpGs denoted by [[CG]];
23. one or more CpGs within DMR 23 as defined by SEQ ID NO: 5023 denoted by CG, preferably wherein the assay is characterised as having an ROC AUC of at least 0.862, and more preferably wherein the panel of one or more CpGs comprises at least the CpGs denoted by [[CG]];
24. one or more CpGs within DMR 24 as defined by SEQ ID NO: 5024 denoted by CG, preferably wherein the assay is characterised as having an ROC AUC of at least 0.861, and more preferably wherein the panel of one or more CpGs comprises at least the CpGs denoted by [[CG]];
25. one or more CpGs within DMR 25 as defined by SEQ ID NO: 5025 denoted by CG, preferably wherein the assay is characterised as having an ROC AUC of at least 0.86, and more preferably wherein the panel of one or more CpGs comprises at least the CpGs denoted by [[CG]];
26. one or more CpGs within DMR 26 as defined by SEQ ID NO: 5026 denoted by CG, preferably wherein the assay is characterised as having an ROC AUC of at least 0.86, and more preferably wherein the panel of one or more CpGs comprises at least the CpGs denoted by [[CG]];
27. one or more CpGs within DMR 27 as defined by SEQ ID NO: 5027 denoted by CG, preferably wherein the assay is characterised as having an ROC AUC of at least 0.86, and more preferably wherein the panel of one or more CpGs comprises at least the CpGs denoted by [[CG]];
28. one or more CpGs within DMR 28 as defined by SEQ ID NO: 5028 denoted by CG, preferably wherein the assay is characterised as having an ROC AUC of at least 0.86, and more preferably wherein the panel of one or more CpGs comprises at least the CpGs denoted by [[CG]];
29. one or more CpGs within DMR 29 as defined by SEQ ID NO: 5029 denoted by CG, preferably wherein the assay is characterised as having an ROC AUC of at least 0.86, and more preferably wherein the panel of one or more CpGs comprises at least the CpGs denoted by [[CG]];
30. one or more CpGs within DMR 30 as defined by SEQ ID NO: 5030 denoted by CG, preferably wherein the assay is characterised as having an ROC AUC of at least 0.859, and more preferably wherein the panel of one or more CpGs comprises at least the CpGs denoted by [[CG]];
31. one or more CpGs within DMR 31 as defined by SEQ ID NO: 5031 denoted by CG, preferably wherein the assay is characterised as having an ROC AUC of at least 0.859, and more preferably wherein the panel of one or more CpGs comprises at least the CpGs denoted by [[CG]];
32. one or more CpGs within DMR 32 as defined by SEQ ID NO: 5032 denoted by CG, preferably wherein the assay is characterised as having an ROC AUC of at least 0.858, and more preferably wherein the panel of one or more CpGs comprises at least the CpGs denoted by [[CG]];
33. one or more CpGs within DMR 33 as defined by SEQ ID NO: 5033 denoted by CG, preferably wherein the assay is characterised as having an ROC AUC of at least 0.858, and more preferably wherein the panel of one or more CpGs comprises at least the CpGs denoted by [[CG]];
34. one or more CpGs within DMR 34 as defined by SEQ ID NO: 5034 denoted by CG, preferably wherein the assay is characterised as having an ROC AUC of at least 0.858, and more preferably wherein the panel of one or more CpGs comprises at least the CpGs denoted by [[CG]];
35. one or more CpGs within DMR 35 as defined by SEQ ID NO: 5035 denoted by CG, preferably wherein the assay is characterised as having an ROC AUC of at least 0.858, and more preferably wherein the panel of one or more CpGs comprises at least the CpGs denoted by [[CG]];
36. one or more CpGs within DMR 36 as defined by SEQ ID NO: 5036 denoted by CG, preferably wherein the assay is characterised as having an ROC AUC of at least 0.858, and more preferably wherein the panel of one or more CpGs comprises at least the CpGs denoted by [[CG]];
37. one or more CpGs within DMR 37 as defined by SEQ ID NO: 5037 denoted by CG, preferably wherein the assay is characterised as having an ROC AUC of at least 0.858, and more preferably wherein the panel of one or more CpGs comprises at least the CpGs denoted by [[CG]];
38. one or more CpGs within DMR 38 as defined by SEQ ID NO: 5038 denoted by CG, preferably wherein the assay is characterised as having an ROC AUC of at least 0.858, and more preferably wherein the panel of one or more CpGs comprises at least the CpGs denoted by [[CG]];
39. one or more CpGs within DMR 39 as defined by SEQ ID NO: 5039 denoted by CG, preferably wherein the assay is characterised as having an ROC AUC of at least 0.858, and more preferably wherein the panel of one or more CpGs comprises at least the CpGs denoted by [[CG]];
40. one or more CpGs within DMR 40 as defined by SEQ ID NO: 5040 denoted by CG, preferably wherein the assay is characterised as having an ROC AUC of at least 0.858, and more preferably wherein the panel of one or more CpGs comprises at least the CpGs denoted by [[CG]];
41. one or more CpGs within DMR 41 as defined by SEQ ID NO: 5041 denoted by CG, preferably wherein the assay is characterised as having an ROC AUC of at least 0.858, and more preferably wherein the panel of one or more CpGs comprises at least the CpGs denoted by [[CG]];
42. one or more CpGs within DMR 42 as defined by SEQ ID NO: 5042 denoted by CG, preferably wherein the assay is characterised as having an ROC AUC of at least 0.858, and more preferably wherein the panel of one or more CpGs comprises at least the CpGs denoted by [[CG]];
43. one or more CpGs within DMR 43 as defined by SEQ ID NO: 5043 denoted by CG, preferably wherein the assay is characterised as having an ROC AUC of at least 0.858, and more preferably wherein the panel of one or more CpGs comprises at least the CpGs denoted by [[CG]];
44. one or more CpGs within DMR 44 as defined by SEQ ID NO: 5044 denoted by CG, preferably wherein the assay is characterised as having an ROC AUC of at least 0.858, and more preferably wherein the panel of one or more CpGs comprises at least the CpGs denoted by [[CG]];
45. one or more CpGs within DMR 45 as defined by SEQ ID NO: 5045 denoted by CG, preferably wherein the assay is characterised as having an ROC AUC of at least 0.858, and more preferably wherein the panel of one or more CpGs comprises at least the CpGs denoted by [[CG]];
46. one or more CpGs within DMR 46 as defined by SEQ ID NO: 5046 denoted by CG, preferably wherein the assay is characterised as having an ROC AUC of at least 0.858, and more preferably wherein the panel of one or more CpGs comprises at least the CpGs denoted by [[CG]];
47. one or more CpGs within DMR 47 as defined by SEQ ID NO: 5047 denoted by CG, preferably wherein the assay is characterised as having an ROC AUC of at least 0.856, and more preferably wherein the panel of one or more CpGs comprises at least the CpGs denoted by [[CG]];
48. one or more CpGs within DMR 48 as defined by SEQ ID NO: 5048 denoted by CG, preferably wherein the assay is characterised as having an ROC AUC of at least 0.855, and more preferably wherein the panel of one or more CpGs comprises at least the CpGs denoted by [[CG]];
49. one or more CpGs within DMR 49 as defined by SEQ ID NO: 5049 denoted by CG, preferably wherein the assay is characterised as having an ROC AUC of at least 0.855, and more preferably wherein the panel of one or more CpGs comprises at least the CpGs denoted by [[CG]];
50. one or more CpGs within DMR 50 as defined by SEQ ID NO: 5050 denoted by CG, preferably wherein the assay is characterised as having an ROC AUC of at least 0.855, and more preferably wherein the panel of one or more CpGs comprises at least the CpGs denoted by [[CG]];
51. one or more CpGs within DMR 51 as defined by SEQ ID NO: 5051 denoted by CG, preferably wherein the assay is characterised as having an ROC AUC of at least 0.852, and more preferably wherein the panel of one or more CpGs comprises at least the CpGs denoted by [[CG]];
52. one or more CpGs within DMR 52 as defined by SEQ ID NO: 5052 denoted by CG, preferably wherein the assay is characterised as having an ROC AUC of at least 0.852, and more preferably wherein the panel of one or more CpGs comprises at least the CpGs denoted by [[CG]];
53. one or more CpGs within DMR 53 as defined by SEQ ID NO: 5053 denoted by CG, preferably wherein the assay is characterised as having an ROC AUC of at least 0.851, and more preferably wherein the panel of one or more CpGs comprises at least the CpGs denoted by [[CG]];
54. one or more CpGs within DMR 54 as defined by SEQ ID NO: 5054 denoted by CG, preferably wherein the assay is characterised as having an ROC AUC of at least 0.851, and more preferably wherein the panel of one or more CpGs comprises at least the CpGs denoted by [[CG]];
55. one or more CpGs within DMR 55 as defined by SEQ ID NO: 5055 denoted by CG, preferably wherein the assay is characterised as having an ROC AUC of at least 0.851, and more preferably wherein the panel of one or more CpGs comprises at least the CpGs denoted by [[CG]];
56. one or more CpGs within DMR 56 as defined by SEQ ID NO: 5056 denoted by CG, preferably wherein the assay is characterised as having an ROC AUC of at least 0.851, and more preferably wherein the panel of one or more CpGs comprises at least the CpGs denoted by [[CG]];
57. one or more CpGs within DMR 57 as defined by SEQ ID NO: 5057 denoted by CG, preferably wherein the assay is characterised as having an ROC AUC of at least 0.851, and more preferably wherein the panel of one or more CpGs comprises at least the CpGs denoted by [[CG]];
58. one or more CpGs within DMR 58 as defined by SEQ ID NO: 5058 denoted by CG, preferably wherein the assay is characterised as having an ROC AUC of at least 0.848, and more preferably wherein the panel of one or more CpGs comprises at least the CpGs denoted by [[CG]];
59. one or more CpGs within DMR 59 as defined by SEQ ID NO: 5059 denoted by CG, preferably wherein the assay is characterised as having an ROC AUC of at least 0.847, and more preferably wherein the panel of one or more CpGs comprises at least the CpGs denoted by [[CG]];
60. one or more CpGs within DMR 60 as defined by SEQ ID NO: 5060 denoted by CG, preferably wherein the assay is characterised as having an ROC AUC of at least 0.847, and more preferably wherein the panel of one or more CpGs comprises at least the CpGs denoted by [[CG]];
61. one or more CpGs within DMR 61 as defined by SEQ ID NO: 5061 denoted by CG, preferably wherein the assay is characterised as having an ROC AUC of at least 0.846, and more preferably wherein the panel of one or more CpGs comprises at least the CpGs denoted by [[CG]];
62. one or more CpGs within DMR 62 as defined by SEQ ID NO: 5062 denoted by CG, preferably wherein the assay is characterised as having an ROC AUC of at least 0.846, and more preferably wherein the panel of one or more CpGs comprises at least the CpGs denoted by [[CG]];
63. one or more CpGs within DMR 63 as defined by SEQ ID NO: 5063 denoted by CG, preferably wherein the assay is characterised as having an ROC AUC of at least 0.845, and more preferably wherein the panel of one or more CpGs comprises at least the CpGs denoted by [[CG]];
64. one or more CpGs within DMR 64 as defined by SEQ ID NO: 5064 denoted by CG, preferably wherein the assay is characterised as having an ROC AUC of at least 0.845, and more preferably wherein the panel of one or more CpGs comprises at least the CpGs denoted by [[CG]];
65. one or more CpGs within DMR 65 as defined by SEQ ID NO: 5065 denoted by CG, preferably wherein the assay is characterised as having an ROC AUC of at least 0.845, and more preferably wherein the panel of one or more CpGs comprises at least the CpGs denoted by [[CG]];
66. one or more CpGs within DMR 66 as defined by SEQ ID NO: 5066 denoted by CG, preferably wherein the assay is characterised as having an ROC AUC of at least 0.845, and more preferably wherein the panel of one or more CpGs comprises at least the CpGs denoted by [[CG]];
67. one or more CpGs within DMR 67 as defined by SEQ ID NO: 5067 denoted by CG, preferably wherein the assay is characterised as having an ROC AUC of at least 0.843, and more preferably wherein the panel of one or more CpGs comprises at least the CpGs denoted by [[CG]];
68. one or more CpGs within DMR 68 as defined by SEQ ID NO: 5068 denoted by CG, preferably wherein the assay is characterised as having an ROC AUC of at least 0.843, and more preferably wherein the panel of one or more CpGs comprises at least the CpGs denoted by [[CG]];
69. one or more CpGs within DMR 69 as defined by SEQ ID NO: 5069 denoted by CG, preferably wherein the assay is characterised as having an ROC AUC of at least 0.84, and more preferably wherein the panel of one or more CpGs comprises at least the CpGs denoted by [[CG]];
70. one or more CpGs within DMR 70 as defined by SEQ ID NO: 5070 denoted by CG, preferably wherein the assay is characterised as having an ROC AUC of at least 0.84, and more preferably wherein the panel of one or more CpGs comprises at least the CpGs denoted by [[CG]];
71. one or more CpGs within DMR 71 as defined by SEQ ID NO: 5071 denoted by CG, preferably wherein the assay is characterised as having an ROC AUC of at least 0.84, and more preferably wherein the panel of one or more CpGs comprises at least the CpGs denoted by [[CG]];
72. one or more CpGs within DMR 72 as defined by SEQ ID NO: 5072 denoted by CG, preferably wherein the assay is characterised as having an ROC AUC of at least 0.839, and more preferably wherein the panel of one or more CpGs comprises at least the CpGs denoted by [[CG]];
73. one or more CpGs within DMR 73 as defined by SEQ ID NO: 5073 denoted by CG, preferably wherein the assay is characterised as having an ROC AUC of at least 0.839, and more preferably wherein the panel of one or more CpGs comprises at least the CpGs denoted by [[CG]];
74. one or more CpGs within DMR 74 as defined by SEQ ID NO: 5074 denoted by CG, preferably wherein the assay is characterised as having an ROC AUC of at least 0.839, and more preferably wherein the panel of one or more CpGs comprises at least the CpGs denoted by [[CG]];
75. one or more CpGs within DMR 75 as defined by SEQ ID NO: 5075 denoted by CG, preferably wherein the assay is characterised as having an ROC AUC of at least 0.839, and more preferably wherein the panel of one or more CpGs comprises at least the CpGs denoted by [[CG]];
76. one or more CpGs within DMR 76 as defined by SEQ ID NO: 5076 denoted by CG, preferably wherein the assay is characterised as having an ROC AUC of at least 0.839, and more preferably wherein the panel of one or more CpGs comprises at least the CpGs denoted by [[CG]];
77. one or more CpGs within DMR 77 as defined by SEQ ID NO: 5077 denoted by CG, preferably wherein the assay is characterised as having an ROC AUC of at least 0.839, and more preferably wherein the panel of one or more CpGs comprises at least the CpGs denoted by [[CG]];
78. one or more CpGs within DMR 78 as defined by SEQ ID NO: 5078 denoted by CG, preferably wherein the assay is characterised as having an ROC AUC of at least 0.838, and more preferably wherein the panel of one or more CpGs comprises at least the CpGs denoted by [[CG]];
79. one or more CpGs within DMR 79 as defined by SEQ ID NO: 5079 denoted by CG, preferably wherein the assay is characterised as having an ROC AUC of at least 0.838, and more preferably wherein the panel of one or more CpGs comprises at least the CpGs denoted by [[CG]];
80. one or more CpGs within DMR 80 as defined by SEQ ID NO: 5080 denoted by CG, preferably wherein the assay is characterised as having an ROC AUC of at least 0.838, and more preferably wherein the panel of one or more CpGs comprises at least the CpGs denoted by [[CG]];
81. one or more CpGs within DMR 81 as defined by SEQ ID NO: 5081 denoted by CG, preferably wherein the assay is characterised as having an ROC AUC of at least 0.838, and more preferably wherein the panel of one or more CpGs comprises at least the CpGs denoted by [[CG]];
82. one or more CpGs within DMR 82 as defined by SEQ ID NO: 5082 denoted by CG, preferably wherein the assay is characterised as having an ROC AUC of at least 0.837, and more preferably wherein the panel of one or more CpGs comprises at least the CpGs denoted by [[CG]];
83. one or more CpGs within DMR 83 as defined by SEQ ID NO: 5083 denoted by CG, preferably wherein the assay is characterised as having an ROC AUC of at least 0.837, and more preferably wherein the panel of one or more CpGs comprises at least the CpGs denoted by [[CG]];
84. one or more CpGs within DMR 84 as defined by SEQ ID NO: 5084 denoted by CG, preferably wherein the assay is characterised as having an ROC AUC of at least 0.837, and more preferably wherein the panel of one or more CpGs comprises at least the CpGs denoted by [[CG]];
85. one or more CpGs within DMR 85 as defined by SEQ ID NO: 5085 denoted by CG, preferably wherein the assay is characterised as having an ROC AUC of at least 0.837, and more preferably wherein the panel of one or more CpGs comprises at least the CpGs denoted by [[CG]];
86. one or more CpGs within DMR 86 as defined by SEQ ID NO: 5086 denoted by CG, preferably wherein the assay is characterised as having an ROC AUC of at least 0.837, and more preferably wherein the panel of one or more CpGs comprises at least the CpGs denoted by [[CG]];
87. one or more CpGs within DMR 87 as defined by SEQ ID NO: 5087 denoted by CG, preferably wherein the assay is characterised as having an ROC AUC of at least 0.837, and more preferably wherein the panel of one or more CpGs comprises at least the CpGs denoted by [[CG]];
88. one or more CpGs within DMR 88 as defined by SEQ ID NO: 5088 denoted by CG, preferably wherein the assay is characterised as having an ROC AUC of at least 0.837, and more preferably wherein the panel of one or more CpGs comprises at least the CpGs denoted by [[CG]];
89. one or more CpGs within DMR 89 as defined by SEQ ID NO: 5089 denoted by CG, preferably wherein the assay is characterised as having an ROC AUC of at least 0.836, and more preferably wherein the panel of one or more CpGs comprises at least the CpGs denoted by [[CG]];
90. one or more CpGs within DMR 90 as defined by SEQ ID NO: 5090 denoted by CG, preferably wherein the assay is characterised as having an ROC AUC of at least 0.836, and more preferably wherein the panel of one or more CpGs comprises at least the CpGs denoted by [[CG]];
91. one or more CpGs within DMR 91 as defined by SEQ ID NO: 5091 denoted by CG, preferably wherein the assay is characterised as having an ROC AUC of at least 0.836, and more preferably wherein the panel of one or more CpGs comprises at least the CpGs denoted by [[CG]];
92. one or more CpGs within DMR 92 as defined by SEQ ID NO: 5092 denoted by CG, preferably wherein the assay is characterised as having an ROC AUC of at least 0.836, and more preferably wherein the panel of one or more CpGs comprises at least the CpGs denoted by [[CG]];
93. one or more CpGs within DMR 93 as defined by SEQ ID NO: 5093 denoted by CG, preferably wherein the assay is characterised as having an ROC AUC of at least 0.835, and more preferably wherein the panel of one or more CpGs comprises at least the CpGs denoted by [[CG]];
94. one or more CpGs within DMR 94 as defined by SEQ ID NO: 5094 denoted by CG, preferably wherein the assay is characterised as having an ROC AUC of at least 0.835, and more preferably wherein the panel of one or more CpGs comprises at least the CpGs denoted by [[CG]];
95. one or more CpGs within DMR 95 as defined by SEQ ID NO: 5095 denoted by CG, preferably wherein the assay is characterised as having an ROC AUC of at least 0.834, and more preferably wherein the panel of one or more CpGs comprises at least the CpGs denoted by [[CG]];
96. one or more CpGs within DMR 96 as defined by SEQ ID NO: 5096 denoted by CG, preferably wherein the assay is characterised as having an ROC AUC of at least 0.834, and more preferably wherein the panel of one or more CpGs comprises at least the CpGs denoted by [[CG]];
97. one or more CpGs within DMR 97 as defined by SEQ ID NO: 5097 denoted by CG, preferably wherein the assay is characterised as having an ROC AUC of at least 0.834, and more preferably wherein the panel of one or more CpGs comprises at least the CpGs denoted by [[CG]];
98. one or more CpGs within DMR 98 as defined by SEQ ID NO: 5098 denoted by CG, preferably wherein the assay is characterised as having an ROC AUC of at least 0.834, and more preferably wherein the panel of one or more CpGs comprises at least the CpGs denoted by [[CG]];
99. one or more CpGs within DMR 99 as defined by SEQ ID NO: 5099 denoted by CG, preferably wherein the assay is characterised as having an ROC AUC of at least 0.834, and more preferably wherein the panel of one or more CpGs comprises at least the CpGs denoted by [[CG]];
100. one or more CpGs within DMR 100 as defined by SEQ ID NO: 5100 denoted by CG, preferably wherein the assay is characterised as having an ROC AUC of at least 0.834, and more preferably wherein the panel of one or more CpGs comprises at least the CpGs denoted by [[CG]];
101. one or more CpGs within DMR 101 as defined by SEQ ID NO: 5101 denoted by CG, preferably wherein the assay is characterised as having an ROC AUC of at least 0.834, and more preferably wherein the panel of one or more CpGs comprises at least the CpGs denoted by [[CG]];
102. one or more CpGs within DMR 102 as defined by SEQ ID NO: 5102 denoted by CG, preferably wherein the assay is characterised as having an ROC AUC of at least 0.834, and more preferably wherein the panel of one or more CpGs comprises at least the CpGs denoted by [[CG]];
103. one or more CpGs within DMR 103 as defined by SEQ ID NO: 5103 denoted by CG, preferably wherein the assay is characterised as having an ROC AUC of at least 0.834, and more preferably wherein the panel of one or more CpGs comprises at least the CpGs denoted by [[CG]];
104. one or more CpGs within DMR 104 as defined by SEQ ID NO: 5104 denoted by CG, preferably wherein the assay is characterised as having an ROC AUC of at least 0.834, and more preferably wherein the panel of one or more CpGs comprises at least the CpGs denoted by [[CG]];
105. one or more CpGs within DMR 105 as defined by SEQ ID NO: 5105 denoted by CG, preferably wherein the assay is characterised as having an ROC AUC of at least 0.834, and more preferably wherein the panel of one or more CpGs comprises at least the CpGs denoted by [[CG]];
106. one or more CpGs within DMR 106 as defined by SEQ ID NO: 5106 denoted by CG, preferably wherein the assay is characterised as having an ROC AUC of at least 0.834, and more preferably wherein the panel of one or more CpGs comprises at least the CpGs denoted by [[CG]];
107. one or more CpGs within DMR 107 as defined by SEQ ID NO: 5107 denoted by CG, preferably wherein the assay is characterised as having an ROC AUC of at least 0.834, and more preferably wherein the panel of one or more CpGs comprises at least the CpGs denoted by [[CG]];
108. one or more CpGs within DMR 108 as defined by SEQ ID NO: 5108 denoted by CG, preferably wherein the assay is characterised as having an ROC AUC of at least 0.833, and more preferably wherein the panel of one or more CpGs comprises at least the CpGs denoted by [[CG]];
109. one or more CpGs within DMR 109 as defined by SEQ ID NO: 5109 denoted by CG, preferably wherein the assay is characterised as having an ROC AUC of at least 0.833, and more preferably wherein the panel of one or more CpGs comprises at least the CpGs denoted by [[CG]];
110. one or more CpGs within DMR 110 as defined by SEQ ID NO: 5110 denoted by CG, preferably wherein the assay is characterised as having an ROC AUC of at least 0.833, and more preferably wherein the panel of one or more CpGs comprises at least the CpGs denoted by [[CG]];
111. one or more CpGs within DMR 111 as defined by SEQ ID NO: 5111 denoted by CG, preferably wherein the assay is characterised as having an ROC AUC of at least 0.833, and more preferably wherein the panel of one or more CpGs comprises at least the CpGs denoted by [[CG]];
112. one or more CpGs within DMR 112 as defined by SEQ ID NO: 5112 denoted by CG, preferably wherein the assay is characterised as having an ROC AUC of at least 0.833, and more preferably wherein the panel of one or more CpGs comprises at least the CpGs denoted by [[CG]];
113. one or more CpGs within DMR 113 as defined by SEQ ID NO: 5113 denoted by CG, preferably wherein the assay is characterised as having an ROC AUC of at least 0.833, and more preferably wherein the panel of one or more CpGs comprises at least the CpGs denoted by [[CG]];
114. one or more CpGs within DMR 114 as defined by SEQ ID NO: 5114 denoted by CG, preferably wherein the assay is characterised as having an ROC AUC of at least 0.833, and more preferably wherein the panel of one or more CpGs comprises at least the CpGs denoted by [[CG]];
115. one or more CpGs within DMR 115 as defined by SEQ ID NO: 5115 denoted by CG, preferably wherein the assay is characterised as having an ROC AUC of at least 0.833, and more preferably wherein the panel of one or more CpGs comprises at least the CpGs denoted by [[CG]];
116. one or more CpGs within DMR 116 as defined by SEQ ID NO: 5116 denoted by CG, preferably wherein the assay is characterised as having an ROC AUC of at least 0.831, and more preferably wherein the panel of one or more CpGs comprises at least the CpGs denoted by [[CG]];
117. one or more CpGs within DMR 117 as defined by SEQ ID NO: 5117 denoted by CG, preferably wherein the assay is characterised as having an ROC AUC of at least 0.831, and more preferably wherein the panel of one or more CpGs comprises at least the CpGs denoted by [[CG]];
118. one or more CpGs within DMR 118 as defined by SEQ ID NO: 5118 denoted by CG, preferably wherein the assay is characterised as having an ROC AUC of at least 0.83, and more preferably wherein the panel of one or more CpGs comprises at least the CpGs denoted by [[CG]];
119. one or more CpGs within DMR 119 as defined by SEQ ID NO: 5119 denoted by CG, preferably wherein the assay is characterised as having an ROC AUC of at least 0.83, and more preferably wherein the panel of one or more CpGs comprises at least the CpGs denoted by [[CG]];
120. one or more CpGs within DMR 120 as defined by SEQ ID NO: 5120 denoted by CG, preferably wherein the assay is characterised as having an ROC AUC of at least 0.83, and more preferably wherein the panel of one or more CpGs comprises at least the CpGs denoted by [[CG]];
121. one or more CpGs within DMR 121 as defined by SEQ ID NO: 5121 denoted by CG, preferably wherein the assay is characterised as having an ROC AUC of at least 0.83, and more preferably wherein the panel of one or more CpGs comprises at least the CpGs denoted by [[CG]];
122. one or more CpGs within DMR 122 as defined by SEQ ID NO: 5122 denoted by CG, preferably wherein the assay is characterised as having an ROC AUC of at least 0.83, and more preferably wherein the panel of one or more CpGs comprises at least the CpGs denoted by [[CG]];
123. one or more CpGs within DMR 123 as defined by SEQ ID NO: 5123 denoted by CG, preferably wherein the assay is characterised as having an ROC AUC of at least 0.83, and more preferably wherein the panel of one or more CpGs comprises at least the CpGs denoted by [[CG]];
124. one or more CpGs within DMR 124 as defined by SEQ ID NO: 5124 denoted by CG, preferably wherein the assay is characterised as having an ROC AUC of at least 0.83, and more preferably wherein the panel of one or more CpGs comprises at least the CpGs denoted by [[CG]];
125. one or more CpGs within DMR 125 as defined by SEQ ID NO: 5125 denoted by CG, preferably wherein the assay is characterised as having an ROC AUC of at least 0.83, and more preferably wherein the panel of one or more CpGs comprises at least the CpGs denoted by [[CG]];
126. one or more CpGs within DMR 126 as defined by SEQ ID NO: 5126 denoted by CG, preferably wherein the assay is characterised as having an ROC AUC of at least 0.83, and more preferably wherein the panel of one or more CpGs comprises at least the CpGs denoted by [[CG]];
127. one or more CpGs within DMR 127 as defined by SEQ ID NO: 5127 denoted by CG, preferably wherein the assay is characterised as having an ROC AUC of at least 0.83, and more preferably wherein the panel of one or more CpGs comprises at least the CpGs denoted by [[CG]];
128. one or more CpGs within DMR 128 as defined by SEQ ID NO: 5128 denoted by CG, preferably wherein the assay is characterised as having an ROC AUC of at least 0.829, and more preferably wherein the panel of one or more CpGs comprises at least the CpGs denoted by [[CG]];
129. one or more CpGs within DMR 129 as defined by SEQ ID NO: 5129 denoted by CG, preferably wherein the assay is characterised as having an ROC AUC of at least 0.829, and more preferably wherein the panel of one or more CpGs comprises at least the CpGs denoted by [[CG]];
130. one or more CpGs within DMR 130 as defined by SEQ ID NO: 5130 denoted by CG, preferably wherein the assay is characterised as having an ROC AUC of at least 0.829, and more preferably wherein the panel of one or more CpGs comprises at least the CpGs denoted by [[CG]];
131. one or more CpGs within DMR 131 as defined by SEQ ID NO: 5131 denoted by CG, preferably wherein the assay is characterised as having an ROC AUC of at least 0.829, and more preferably wherein the panel of one or more CpGs comprises at least the CpGs denoted by [[CG]];
132. one or more CpGs within DMR 132 as defined by SEQ ID NO: 5132 denoted by CG, preferably wherein the assay is characterised as having an ROC AUC of at least 0.828, and more preferably wherein the panel of one or more CpGs comprises at least the CpGs denoted by [[CG]];
133. one or more CpGs within DMR 133 as defined by SEQ ID NO: 5133 denoted by CG, preferably wherein the assay is characterised as having an ROC AUC of at least 0.828, and more preferably wherein the panel of one or more CpGs comprises at least the CpGs denoted by [[CG]];
134. one or more CpGs within DMR 134 as defined by SEQ ID NO: 5134 denoted by CG, preferably wherein the assay is characterised as having an ROC AUC of at least 0.828, and more preferably wherein the panel of one or more CpGs comprises at least the CpGs denoted by [[CG]];
135. one or more CpGs within DMR 135 as defined by SEQ ID NO: 5135 denoted by CG, preferably wherein the assay is characterised as having an ROC AUC of at least 0.828, and more preferably wherein the panel of one or more CpGs comprises at least the CpGs denoted by [[CG]];
136. one or more CpGs within DMR 136 as defined by SEQ ID NO: 5136 denoted by CG, preferably wherein the assay is characterised as having an ROC AUC of at least 0.828, and more preferably wherein the panel of one or more CpGs comprises at least the CpGs denoted by [[CG]];
137. one or more CpGs within DMR 137 as defined by SEQ ID NO: 5137 denoted by CG, preferably wherein the assay is characterised as having an ROC AUC of at least 0.827, and more preferably wherein the panel of one or more CpGs comprises at least the CpGs denoted by [[CG]];
138. one or more CpGs within DMR 138 as defined by SEQ ID NO: 5138 denoted by CG, preferably wherein the assay is characterised as having an ROC AUC of at least 0.827, and more preferably wherein the panel of one or more CpGs comprises at least the CpGs denoted by [[CG]];
139. one or more CpGs within DMR 139 as defined by SEQ ID NO: 5139 denoted by CG, preferably wherein the assay is characterised as having an ROC AUC of at least 0.827, and more preferably wherein the panel of one or more CpGs comprises at least the CpGs denoted by [[CG]];
140. one or more CpGs within DMR 140 as defined by SEQ ID NO: 5140 denoted by CG, preferably wherein the assay is characterised as having an ROC AUC of at least 0.826, and more preferably wherein the panel of one or more CpGs comprises at least the CpGs denoted by [[CG]];
141. one or more CpGs within DMR 141 as defined by SEQ ID NO: 5141 denoted by CG, preferably wherein the assay is characterised as having an ROC AUC of at least 0.826, and more preferably wherein the panel of one or more CpGs comprises at least the CpGs denoted by [[CG]];
142. one or more CpGs within DMR 142 as defined by SEQ ID NO: 5142 denoted by CG, preferably wherein the assay is characterised as having an ROC AUC of at least 0.826, and more preferably wherein the panel of one or more CpGs comprises at least the CpGs denoted by [[CG]];
143. one or more CpGs within DMR 143 as defined by SEQ ID NO: 5143 denoted by CG, preferably wherein the assay is characterised as having an ROC AUC of at least 0.826, and more preferably wherein the panel of one or more CpGs comprises at least the CpGs denoted by [[CG]];
144. one or more CpGs within DMR 144 as defined by SEQ ID NO: 5144 denoted by CG, preferably wherein the assay is characterised as having an ROC AUC of at least 0.826, and more preferably wherein the panel of one or more CpGs comprises at least the CpGs denoted by [[CG]];
145. one or more CpGs within DMR 145 as defined by SEQ ID NO: 5145 denoted by CG, preferably wherein the assay is characterised as having an ROC AUC of at least 0.826, and more preferably wherein the panel of one or more CpGs comprises at least the CpGs denoted by [[CG]];
146. one or more CpGs within DMR 146 as defined by SEQ ID NO: 5146 denoted by CG, preferably wherein the assay is characterised as having an ROC AUC of at least 0.826, and more preferably wherein the panel of one or more CpGs comprises at least the CpGs denoted by [[CG]];
147. one or more CpGs within DMR 147 as defined by SEQ ID NO: 5147 denoted by CG, preferably wherein the assay is characterised as having an ROC AUC of at least 0.825, and more preferably wherein the panel of one or more CpGs comprises at least the CpGs denoted by [[CG]];
148. one or more CpGs within DMR 148 as defined by SEQ ID NO: 5148 denoted by CG, preferably wherein the assay is characterised as having an ROC AUC of at least 0.825, and more preferably wherein the panel of one or more CpGs comprises at least the CpGs denoted by [[CG]];
149. one or more CpGs within DMR 149 as defined by SEQ ID NO: 5149 denoted by CG, preferably wherein the assay is characterised as having an ROC AUC of at least 0.825, and more preferably wherein the panel of one or more CpGs comprises at least the CpGs denoted by [[CG]];
150. one or more CpGs within DMR 150 as defined by SEQ ID NO: 5150 denoted by CG, preferably wherein the assay is characterised as having an ROC AUC of at least 0.825, and more preferably wherein the panel of one or more CpGs comprises at least the CpGs denoted by [[CG]];
151. one or more CpGs within DMR 151 as defined by SEQ ID NO: 5151 denoted by CG, preferably wherein the assay is characterised as having an ROC AUC of at least 0.825, and more preferably wherein the panel of one or more CpGs comprises at least the CpGs denoted by [[CG]];
152. one or more CpGs within DMR 152 as defined by SEQ ID NO: 5152 denoted by CG, preferably wherein the assay is characterised as having an ROC AUC of at least 0.825, and more preferably wherein the panel of one or more CpGs comprises at least the CpGs denoted by [[CG]];
153. one or more CpGs within DMR 153 as defined by SEQ ID NO: 5153 denoted by CG, preferably wherein the assay is characterised as having an ROC AUC of at least 0.825, and more preferably wherein the panel of one or more CpGs comprises at least the CpGs denoted by [[CG]];
154. one or more CpGs within DMR 154 as defined by SEQ ID NO: 5154 denoted by CG, preferably wherein the assay is characterised as having an ROC AUC of at least 0.825, and more preferably wherein the panel of one or more CpGs comprises at least the CpGs denoted by [[CG]];
155. one or more CpGs within DMR 155 as defined by SEQ ID NO: 5155 denoted by CG, preferably wherein the assay is characterised as having an ROC AUC of at least 0.825, and more preferably wherein the panel of one or more CpGs comprises at least the CpGs denoted by [[CG]];
156. one or more CpGs within DMR 156 as defined by SEQ ID NO: 5156 denoted by CG, preferably wherein the assay is characterised as having an ROC AUC of at least 0.825, and more preferably wherein the panel of one or more CpGs comprises at least the CpGs denoted by [[CG]];
157. one or more CpGs within DMR 157 as defined by SEQ ID NO: 5157 denoted by CG, preferably wherein the assay is characterised as having an ROC AUC of at least 0.825, and more preferably wherein the panel of one or more CpGs comprises at least the CpGs denoted by [[CG]];
158. one or more CpGs within DMR 158 as defined by SEQ ID NO: 5158 denoted by CG, preferably wherein the assay is characterised as having an ROC AUC of at least 0.825, and more preferably wherein the panel of one or more CpGs comprises at least the CpGs denoted by [[CG]];
159. one or more CpGs within DMR 159 as defined by SEQ ID NO: 5159 denoted by CG, preferably wherein the assay is characterised as having an ROC AUC of at least 0.825, and more preferably wherein the panel of one or more CpGs comprises at least the CpGs denoted by [[CG]];
160. one or more CpGs within DMR 160 as defined by SEQ ID NO: 5160 denoted by CG, preferably wherein the assay is characterised as having an ROC AUC of at least 0.825, and more preferably wherein the panel of one or more CpGs comprises at least the CpGs denoted by [[CG]];
161. one or more CpGs within DMR 161 as defined by SEQ ID NO: 5161 denoted by CG, preferably wherein the assay is characterised as having an ROC AUC of at least 0.825, and more preferably wherein the panel of one or more CpGs comprises at least the CpGs denoted by [[CG]];
162. one or more CpGs within DMR 162 as defined by SEQ ID NO: 5162 denoted by CG, preferably wherein the assay is characterised as having an ROC AUC of at least 0.825, and more preferably wherein the panel of one or more CpGs comprises at least the CpGs denoted by [[CG]];
163. one or more CpGs within DMR 163 as defined by SEQ ID NO: 5163 denoted by CG, preferably wherein the assay is characterised as having an ROC AUC of at least 0.825, and more preferably wherein the panel of one or more CpGs comprises at least the CpGs denoted by [[CG]];
164. one or more CpGs within DMR 164 as defined by SEQ ID NO: 5164 denoted by CG, preferably wherein the assay is characterised as having an ROC AUC of at least 0.824, and more preferably wherein the panel of one or more CpGs comprises at least the CpGs denoted by [[CG]];
165. one or more CpGs within DMR 165 as defined by SEQ ID NO: 5165 denoted by CG, preferably wherein the assay is characterised as having an ROC AUC of at least 0.823, and more preferably wherein the panel of one or more CpGs comprises at least the CpGs denoted by [[CG]];
166. one or more CpGs within DMR 166 as defined by SEQ ID NO: 5166 denoted by CG, preferably wherein the assay is characterised as having an ROC AUC of at least 0.823, and more preferably wherein the panel of one or more CpGs comprises at least the CpGs denoted by [[CG]];
167. one or more CpGs within DMR 167 as defined by SEQ ID NO: 5167 denoted by CG, preferably wherein the assay is characterised as having an ROC AUC of at least 0.823, and more preferably wherein the panel of one or more CpGs comprises at least the CpGs denoted by [[CG]];
168. one or more CpGs within DMR 168 as defined by SEQ ID NO: 5168 denoted by CG, preferably wherein the assay is characterised as having an ROC AUC of at least 0.823, and more preferably wherein the panel of one or more CpGs comprises at least the CpGs denoted by [[CG]];
169. one or more CpGs within DMR 169 as defined by SEQ ID NO: 5169 denoted by CG, preferably wherein the assay is characterised as having an ROC AUC of at least 0.823, and more preferably wherein the panel of one or more CpGs comprises at least the CpGs denoted by [[CG]];
170. one or more CpGs within DMR 170 as defined by SEQ ID NO: 5170 denoted by CG, preferably wherein the assay is characterised as having an ROC AUC of at least 0.823, and more preferably wherein the panel of one or more CpGs comprises at least the CpGs denoted by [[CG]];
171. one or more CpGs within DMR 171 as defined by SEQ ID NO: 5171 denoted by CG, preferably wherein the assay is characterised as having an ROC AUC of at least 0.823, and more preferably wherein the panel of one or more CpGs comprises at least the CpGs denoted by [[CG]];
172. one or more CpGs within DMR 172 as defined by SEQ ID NO: 5172 denoted by CG, preferably wherein the assay is characterised as having an ROC AUC of at least 0.823, and more preferably wherein the panel of one or more CpGs comprises at least the CpGs denoted by [[CG]];
173. one or more CpGs within DMR 173 as defined by SEQ ID NO: 5173 denoted by CG, preferably wherein the assay is characterised as having an ROC AUC of at least 0.823, and more preferably wherein the panel of one or more CpGs comprises at least the CpGs denoted by [[CG]];
174. one or more CpGs within DMR 174 as defined by SEQ ID NO: 5174 denoted by CG, preferably wherein the assay is characterised as having an ROC AUC of at least 0.822, and more preferably wherein the panel of one or more CpGs comprises at least the CpGs denoted by [[CG]];
175. one or more CpGs within DMR 175 as defined by SEQ ID NO: 5175 denoted by CG, preferably wherein the assay is characterised as having an ROC AUC of at least 0.822, and more preferably wherein the panel of one or more CpGs comprises at least the CpGs denoted by [[CG]];
176. one or more CpGs within DMR 176 as defined by SEQ ID NO: 5176 denoted by CG, preferably wherein the assay is characterised as having an ROC AUC of at least 0.822, and more preferably wherein the panel of one or more CpGs comprises at least the CpGs denoted by [[CG]];
177. one or more CpGs within DMR 177 as defined by SEQ ID NO: 5177 denoted by CG, preferably wherein the assay is characterised as having an ROC AUC of at least 0.822, and more preferably wherein the panel of one or more CpGs comprises at least the CpGs denoted by [[CG]];
178. one or more CpGs within DMR 178 as defined by SEQ ID NO: 5178 denoted by CG, preferably wherein the assay is characterised as having an ROC AUC of at least 0.822, and more preferably wherein the panel of one or more CpGs comprises at least the CpGs denoted by [[CG]];
179. one or more CpGs within DMR 179 as defined by SEQ ID NO: 5179 denoted by CG, preferably wherein the assay is characterised as having an ROC AUC of at least 0.822, and more preferably wherein the panel of one or more CpGs comprises at least the CpGs denoted by [[CG]];
180. one or more CpGs within DMR 180 as defined by SEQ ID NO: 5180 denoted by CG, preferably wherein the assay is characterised as having an ROC AUC of at least 0.821, and more preferably wherein the panel of one or more CpGs comprises at least the CpGs denoted by [[CG]];
181. one or more CpGs within DMR 181 as defined by SEQ ID NO: 5181 denoted by CG, preferably wherein the assay is characterised as having an ROC AUC of at least 0.821, and more preferably wherein the panel of one or more CpGs comprises at least the CpGs denoted by [[CG]];
182. one or more CpGs within DMR 182 as defined by SEQ ID NO: 5182 denoted by CG, preferably wherein the assay is characterised as having an ROC AUC of at least 0.821, and more preferably wherein the panel of one or more CpGs comprises at least the CpGs denoted by [[CG]];
183. one or more CpGs within DMR 183 as defined by SEQ ID NO: 5183 denoted by CG, preferably wherein the assay is characterised as having an ROC AUC of at least 0.821, and more preferably wherein the panel of one or more CpGs comprises at least the CpGs denoted by [[CG]];
184. one or more CpGs within DMR 184 as defined by SEQ ID NO: 5184 denoted by CG, preferably wherein the assay is characterised as having an ROC AUC of at least 0.821, and more preferably wherein the panel of one or more CpGs comprises at least the CpGs denoted by [[CG]];
185. one or more CpGs within DMR 185 as defined by SEQ ID NO: 5185 denoted by CG, preferably wherein the assay is characterised as having an ROC AUC of at least 0.821, and more preferably wherein the panel of one or more CpGs comprises at least the CpGs denoted by [[CG]];
186. one or more CpGs within DMR 186 as defined by SEQ ID NO: 5186 denoted by CG, preferably wherein the assay is characterised as having an ROC AUC of at least 0.821, and more preferably wherein the panel of one or more CpGs comprises at least the CpGs denoted by [[CG]];
187. one or more CpGs within DMR 187 as defined by SEQ ID NO: 5187 denoted by CG, preferably wherein the assay is characterised as having an ROC AUC of at least 0.821, and more preferably wherein the panel of one or more CpGs comprises at least the CpGs denoted by [[CG]];
188. one or more CpGs within DMR 188 as defined by SEQ ID NO: 5188 denoted by CG, preferably wherein the assay is characterised as having an ROC AUC of at least 0.821, and more preferably wherein the panel of one or more CpGs comprises at least the CpGs denoted by [[CG]];
189. one or more CpGs within DMR 189 as defined by SEQ ID NO: 5189 denoted by CG, preferably wherein the assay is characterised as having an ROC AUC of at least 0.821, and more preferably wherein the panel of one or more CpGs comprises at least the CpGs denoted by [[CG]];
190. one or more CpGs within DMR 190 as defined by SEQ ID NO: 5190 denoted by CG, preferably wherein the assay is characterised as having an ROC AUC of at least 0.82, and more preferably wherein the panel of one or more CpGs comprises at least the CpGs denoted by [[CG]];
191. one or more CpGs within DMR 191 as defined by SEQ ID NO: 5191 denoted by CG, preferably wherein the assay is characterised as having an ROC AUC of at least 0.82, and more preferably wherein the panel of one or more CpGs comprises at least the CpGs denoted by [[CG]];
192. one or more CpGs within DMR 192 as defined by SEQ ID NO: 5192 denoted by CG, preferably wherein the assay is characterised as having an ROC AUC of at least 0.82, and more preferably wherein the panel of one or more CpGs comprises at least the CpGs denoted by [[CG]];
193. one or more CpGs within DMR 193 as defined by SEQ ID NO: 5193 denoted by CG, preferably wherein the assay is characterised as having an ROC AUC of at least 0.82, and more preferably wherein the panel of one or more CpGs comprises at least the CpGs denoted by [[CG]];
194. one or more CpGs within DMR 194 as defined by SEQ ID NO: 5194 denoted by CG, preferably wherein the assay is characterised as having an ROC AUC of at least 0.819, and more preferably wherein the panel of one or more CpGs comprises at least the CpGs denoted by [[CG]];
195. one or more CpGs within DMR 195 as defined by SEQ ID NO: 5195 denoted by CG, preferably wherein the assay is characterised as having an ROC AUC of at least 0.819, and more preferably wherein the panel of one or more CpGs comprises at least the CpGs denoted by [[CG]];
196. one or more CpGs within DMR 196 as defined by SEQ ID NO: 5196 denoted by CG, preferably wherein the assay is characterised as having an ROC AUC of at least 0.819, and more preferably wherein the panel of one or more CpGs comprises at least the CpGs denoted by [[CG]];
197. one or more CpGs within DMR 197 as defined by SEQ ID NO: 5197 denoted by CG, preferably wherein the assay is characterised as having an ROC AUC of at least 0.819, and more preferably wherein the panel of one or more CpGs comprises at least the CpGs denoted by [[CG]];
198. one or more CpGs within DMR 198 as defined by SEQ ID NO: 5198 denoted by CG, preferably wherein the assay is characterised as having an ROC AUC of at least 0.819, and more preferably wherein the panel of one or more CpGs comprises at least the CpGs denoted by [[CG]];
199. one or more CpGs within DMR 199 as defined by SEQ ID NO: 5199 denoted by CG, preferably wherein the assay is characterised as having an ROC AUC of at least 0.819, and more preferably wherein the panel of one or more CpGs comprises at least the CpGs denoted by [[CG]];
200. one or more CpGs within DMR 200 as defined by SEQ ID NO: 5200 denoted by CG, preferably wherein the assay is characterised as having an ROC AUC of at least 0.819, and more preferably wherein the panel of one or more CpGs comprises at least the CpGs denoted by [[CG]];
201. one or more CpGs within DMR 201 as defined by SEQ ID NO: 5201 denoted by CG, preferably wherein the assay is characterised as having an ROC AUC of at least 0.819, and more preferably wherein the panel of one or more CpGs comprises at least the CpGs denoted by [[CG]];
202. one or more CpGs within DMR 202 as defined by SEQ ID NO: 5202 denoted by CG, preferably wherein the assay is characterised as having an ROC AUC of at least 0.819, and more preferably wherein the panel of one or more CpGs comprises at least the CpGs denoted by [[CG]];
203. one or more CpGs within DMR 203 as defined by SEQ ID NO: 5203 denoted by CG, preferably wherein the assay is characterised as having an ROC AUC of at least 0.819, and more preferably wherein the panel of one or more CpGs comprises at least the CpGs denoted by [[CG]];
204. one or more CpGs within DMR 204 as defined by SEQ ID NO: 5204 denoted by CG, preferably wherein the assay is characterised as having an ROC AUC of at least 0.818, and more preferably wherein the panel of one or more CpGs comprises at least the CpGs denoted by [[CG]];
205. one or more CpGs within DMR 205 as defined by SEQ ID NO: 5205 denoted by CG, preferably wherein the assay is characterised as having an ROC AUC of at least 0.818, and more preferably wherein the panel of one or more CpGs comprises at least the CpGs denoted by [[CG]];
206. one or more CpGs within DMR 206 as defined by SEQ ID NO: 5206 denoted by CG, preferably wherein the assay is characterised as having an ROC AUC of at least 0.818, and more preferably wherein the panel of one or more CpGs comprises at least the CpGs denoted by [[CG]];
207. one or more CpGs within DMR 207 as defined by SEQ ID NO: 5207 denoted by CG, preferably wherein the assay is characterised as having an ROC AUC of at least 0.818, and more preferably wherein the panel of one or more CpGs comprises at least the CpGs denoted by [[CG]];
208. one or more CpGs within DMR 208 as defined by SEQ ID NO: 5208 denoted by CG, preferably wherein the assay is characterised as having an ROC AUC of at least 0.818, and more preferably wherein the panel of one or more CpGs comprises at least the CpGs denoted by [[CG]];
209. one or more CpGs within DMR 209 as defined by SEQ ID NO: 5209 denoted by CG, preferably wherein the assay is characterised as having an ROC AUC of at least 0.818, and more preferably wherein the panel of one or more CpGs comprises at least the CpGs denoted by [[CG]];
210. one or more CpGs within DMR 210 as defined by SEQ ID NO: 5210 denoted by CG, preferably wherein the assay is characterised as having an ROC AUC of at least 0.818, and more preferably wherein the panel of one or more CpGs comprises at least the CpGs denoted by [[CG]];
211. one or more CpGs within DMR 211 as defined by SEQ ID NO: 5211 denoted by CG, preferably wherein the assay is characterised as having an ROC AUC of at least 0.817, and more preferably wherein the panel of one or more CpGs comprises at least the CpGs denoted by [[CG]];
212. one or more CpGs within DMR 212 as defined by SEQ ID NO: 5212 denoted by CG, preferably wherein the assay is characterised as having an ROC AUC of at least 0.817, and more preferably wherein the panel of one or more CpGs comprises at least the CpGs denoted by [[CG]];
213. one or more CpGs within DMR 213 as defined by SEQ ID NO: 5213 denoted by CG, preferably wherein the assay is characterised as having an ROC AUC of at least 0.817, and more preferably wherein the panel of one or more CpGs comprises at least the CpGs denoted by [[CG]];
214. one or more CpGs within DMR 214 as defined by SEQ ID NO: 5214 denoted by CG, preferably wherein the assay is characterised as having an ROC AUC of at least 0.817, and more preferably wherein the panel of one or more CpGs comprises at least the CpGs denoted by [[CG]];
215. one or more CpGs within DMR 215 as defined by SEQ ID NO: 5215 denoted by CG, preferably wherein the assay is characterised as having an ROC AUC of at least 0.817, and more preferably wherein the panel of one or more CpGs comprises at least the CpGs denoted by [[CG]];
216. one or more CpGs within DMR 216 as defined by SEQ ID NO: 5216 denoted by CG, preferably wherein the assay is characterised as having an ROC AUC of at least 0.817, and more preferably wherein the panel of one or more CpGs comprises at least the CpGs denoted by [[CG]];
217. one or more CpGs within DMR 217 as defined by SEQ ID NO: 5217 denoted by CG, preferably wherein the assay is characterised as having an ROC AUC of at least 0.817, and more preferably wherein the panel of one or more CpGs comprises at least the CpGs denoted by [[CG]];
218. one or more CpGs within DMR 218 as defined by SEQ ID NO: 5218 denoted by CG, preferably wherein the assay is characterised as having an ROC AUC of at least 0.817, and more preferably wherein the panel of one or more CpGs comprises at least the CpGs denoted by [[CG]];
219. one or more CpGs within DMR 219 as defined by SEQ ID NO: 5219 denoted by CG, preferably wherein the assay is characterised as having an ROC AUC of at least 0.817, and more preferably wherein the panel of one or more CpGs comprises at least the CpGs denoted by [[CG]];
220. one or more CpGs within DMR 220 as defined by SEQ ID NO: 5220 denoted by CG, preferably wherein the assay is characterised as having an ROC AUC of at least 0.817, and more preferably wherein the panel of one or more CpGs comprises at least the CpGs denoted by [[CG]];
221. one or more CpGs within DMR 221 as defined by SEQ ID NO: 5221 denoted by CG, preferably wherein the assay is characterised as having an ROC AUC of at least 0.816, and more preferably wherein the panel of one or more CpGs comprises at least the CpGs denoted by [[CG]];
222. one or more CpGs within DMR 222 as defined by SEQ ID NO: 5222 denoted by CG, preferably wherein the assay is characterised as having an ROC AUC of at least 0.816, and more preferably wherein the panel of one or more CpGs comprises at least the CpGs denoted by [[CG]];
223. one or more CpGs within DMR 223 as defined by SEQ ID NO: 5223 denoted by CG, preferably wherein the assay is characterised as having an ROC AUC of at least 0.816, and more preferably wherein the panel of one or more CpGs comprises at least the CpGs denoted by [[CG]];
224. one or more CpGs within DMR 224 as defined by SEQ ID NO: 5224 denoted by CG, preferably wherein the assay is characterised as having an ROC AUC of at least 0.815, and more preferably wherein the panel of one or more CpGs comprises at least the CpGs denoted by [[CG]];
225. one or more CpGs within DMR 225 as defined by SEQ ID NO: 5225 denoted by CG, preferably wherein the assay is characterised as having an ROC AUC of at least 0.815, and more preferably wherein the panel of one or more CpGs comprises at least the CpGs denoted by [[CG]];
226. one or more CpGs within DMR 226 as defined by SEQ ID NO: 5226 denoted by CG, preferably wherein the assay is characterised as having an ROC AUC of at least 0.815, and more preferably wherein the panel of one or more CpGs comprises at least the CpGs denoted by [[CG]];
227. one or more CpGs within DMR 227 as defined by SEQ ID NO: 5227 denoted by CG, preferably wherein the assay is characterised as having an ROC AUC of at least 0.814, and more preferably wherein the panel of one or more CpGs comprises at least the CpGs denoted by [[CG]];
228. one or more CpGs within DMR 228 as defined by SEQ ID NO: 5228 denoted by CG, preferably wherein the assay is characterised as having an ROC AUC of at least 0.814, and more preferably wherein the panel of one or more CpGs comprises at least the CpGs denoted by [[CG]];
229. one or more CpGs within DMR 229 as defined by SEQ ID NO: 5229 denoted by CG, preferably wherein the assay is characterised as having an ROC AUC of at least 0.814, and more preferably wherein the panel of one or more CpGs comprises at least the CpGs denoted by [[CG]];
230. one or more CpGs within DMR 230 as defined by SEQ ID NO: 5230 denoted by CG, preferably wherein the assay is characterised as having an ROC AUC of at least 0.814, and more preferably wherein the panel of one or more CpGs comprises at least the CpGs denoted by [[CG]];
231. one or more CpGs within DMR 231 as defined by SEQ ID NO: 5231 denoted by CG, preferably wherein the assay is characterised as having an ROC AUC of at least 0.814, and more preferably wherein the panel of one or more CpGs comprises at least the CpGs denoted by [[CG]];
232. one or more CpGs within DMR 232 as defined by SEQ ID NO: 5232 denoted by CG, preferably wherein the assay is characterised as having an ROC AUC of at least 0.814, and more preferably wherein the panel of one or more CpGs comprises at least the CpGs denoted by [[CG]];
233. one or more CpGs within DMR 233 as defined by SEQ ID NO: 5233 denoted by CG, preferably wherein the assay is characterised as having an ROC AUC of at least 0.814, and more preferably wherein the panel of one or more CpGs comprises at least the CpGs denoted by [[CG]];
234. one or more CpGs within DMR 234 as defined by SEQ ID NO: 5234 denoted by CG, preferably wherein the assay is characterised as having an ROC AUC of at least 0.814, and more preferably wherein the panel of one or more CpGs comprises at least the CpGs denoted by [[CG]];
235. one or more CpGs within DMR 235 as defined by SEQ ID NO: 5235 denoted by CG, preferably wherein the assay is characterised as having an ROC AUC of at least 0.814, and more preferably wherein the panel of one or more CpGs comprises at least the CpGs denoted by [[CG]];
236. one or more CpGs within DMR 236 as defined by SEQ ID NO: 5236 denoted by CG, preferably wherein the assay is characterised as having an ROC AUC of at least 0.814, and more preferably wherein the panel of one or more CpGs comprises at least the CpGs denoted by [[CG]];
237. one or more CpGs within DMR 237 as defined by SEQ ID NO: 5237 denoted by CG, preferably wherein the assay is characterised as having an ROC AUC of at least 0.814, and more preferably wherein the panel of one or more CpGs comprises at least the CpGs denoted by [[CG]];
238. one or more CpGs within DMR 238 as defined by SEQ ID NO: 5238 denoted by CG, preferably wherein the assay is characterised as having an ROC AUC of at least 0.813, and more preferably wherein the panel of one or more CpGs comprises at least the CpGs denoted by [[CG]];
239. one or more CpGs within DMR 239 as defined by SEQ ID NO: 5239 denoted by CG, preferably wherein the assay is characterised as having an ROC AUC of at least 0.813, and more preferably wherein the panel of one or more CpGs comprises at least the CpGs denoted by [[CG]];
240. one or more CpGs within DMR 240 as defined by SEQ ID NO: 5240 denoted by CG, preferably wherein the assay is characterised as having an ROC AUC of at least 0.813, and more preferably wherein the panel of one or more CpGs comprises at least the CpGs denoted by [[CG]];
241. one or more CpGs within DMR 241 as defined by SEQ ID NO: 5241 denoted by CG, preferably wherein the assay is characterised as having an ROC AUC of at least 0.813, and more preferably wherein the panel of one or more CpGs comprises at least the CpGs denoted by [[CG]];
242. one or more CpGs within DMR 242 as defined by SEQ ID NO: 5242 denoted by CG, preferably wherein the assay is characterised as having an ROC AUC of at least 0.812, and more preferably wherein the panel of one or more CpGs comprises at least the CpGs denoted by [[CG]];
243. one or more CpGs within DMR 243 as defined by SEQ ID NO: 5243 denoted by CG, preferably wherein the assay is characterised as having an ROC AUC of at least 0.812, and more preferably wherein the panel of one or more CpGs comprises at least the CpGs denoted by [[CG]];
244. one or more CpGs within DMR 244 as defined by SEQ ID NO: 5244 denoted by CG, preferably wherein the assay is characterised as having an ROC AUC of at least 0.812, and more preferably wherein the panel of one or more CpGs comprises at least the CpGs denoted by [[CG]];
245. one or more CpGs within DMR 245 as defined by SEQ ID NO: 5245 denoted by CG, preferably wherein the assay is characterised as having an ROC AUC of at least 0.812, and more preferably wherein the panel of one or more CpGs comprises at least the CpGs denoted by [[CG]];
246. one or more CpGs within DMR 246 as defined by SEQ ID NO: 5246 denoted by CG, preferably wherein the assay is characterised as having an ROC AUC of at least 0.812, and more preferably wherein the panel of one or more CpGs comprises at least the CpGs denoted by [[CG]];
247. one or more CpGs within DMR 247 as defined by SEQ ID NO: 5247 denoted by CG, preferably wherein the assay is characterised as having an ROC AUC of at least 0.812, and more preferably wherein the panel of one or more CpGs comprises at least the CpGs denoted by [[CG]];
248. one or more CpGs within DMR 248 as defined by SEQ ID NO: 5248 denoted by CG, preferably wherein the assay is characterised as having an ROC AUC of at least 0.811, and more preferably wherein the panel of one or more CpGs comprises at least the CpGs denoted by [[CG]];
249. one or more CpGs within DMR 249 as defined by SEQ ID NO: 5249 denoted by CG, preferably wherein the assay is characterised as having an ROC AUC of at least 0.811, and more preferably wherein the panel of one or more CpGs comprises at least the CpGs denoted by [[CG]];
250. one or more CpGs within DMR 250 as defined by SEQ ID NO: 5250 denoted by CG, preferably wherein the assay is characterised as having an ROC AUC of at least 0.811, and more preferably wherein the panel of one or more CpGs comprises at least the CpGs denoted by [[CG]];
251. one or more CpGs within DMR 251 as defined by SEQ ID NO: 5251 denoted by CG, preferably wherein the assay is characterised as having an ROC AUC of at least 0.811, and more preferably wherein the panel of one or more CpGs comprises at least the CpGs denoted by [[CG]];
252. one or more CpGs within DMR 252 as defined by SEQ ID NO: 5252 denoted by CG, preferably wherein the assay is characterised as having an ROC AUC of at least 0.81, and more preferably wherein the panel of one or more CpGs comprises at least the CpGs denoted by [[CG]];
253. one or more CpGs within DMR 253 as defined by SEQ ID NO: 5253 denoted by CG, preferably wherein the assay is characterised as having an ROC AUC of at least 0.81, and more preferably wherein the panel of one or more CpGs comprises at least the CpGs denoted by [[CG]];
254. one or more CpGs within DMR 254 as defined by SEQ ID NO: 5254 denoted by CG, preferably wherein the assay is characterised as having an ROC AUC of at least 0.81, and more preferably wherein the panel of one or more CpGs comprises at least the CpGs denoted by [[CG]];
255. one or more CpGs within DMR 255 as defined by SEQ ID NO: 5255 denoted by CG, preferably wherein the assay is characterised as having an ROC AUC of at least 0.81, and more preferably wherein the panel of one or more CpGs comprises at least the CpGs denoted by [[CG]];
256. one or more CpGs within DMR 256 as defined by SEQ ID NO: 5256 denoted by CG, preferably wherein the assay is characterised as having an ROC AUC of at least 0.81, and more preferably wherein the panel of one or more CpGs comprises at least the CpGs denoted by [[CG]];
257. one or more CpGs within DMR 257 as defined by SEQ ID NO: 5257 denoted by CG, preferably wherein the assay is characterised as having an ROC AUC of at least 0.81, and more preferably wherein the panel of one or more CpGs comprises at least the CpGs denoted by [[CG]];
258. one or more CpGs within DMR 258 as defined by SEQ ID NO: 5258 denoted by CG, preferably wherein the assay is characterised as having an ROC AUC of at least 0.809, and more preferably wherein the panel of one or more CpGs comprises at least the CpGs denoted by [[CG]];
259. one or more CpGs within DMR 259 as defined by SEQ ID NO: 5259 denoted by CG, preferably wherein the assay is characterised as having an ROC AUC of at least 0.809, and more preferably wherein the panel of one or more CpGs comprises at least the CpGs denoted by [[CG]];
260. one or more CpGs within DMR 260 as defined by SEQ ID NO: 5260 denoted by CG, preferably wherein the assay is characterised as having an ROC AUC of at least 0.809, and more preferably wherein the panel of one or more CpGs comprises at least the CpGs denoted by [[CG]];
261. one or more CpGs within DMR 261 as defined by SEQ ID NO: 5261 denoted by CG, preferably wherein the assay is characterised as having an ROC AUC of at least 0.809, and more preferably wherein the panel of one or more CpGs comprises at least the CpGs denoted by [[CG]];
262. one or more CpGs within DMR 262 as defined by SEQ ID NO: 5262 denoted by CG, preferably wherein the assay is characterised as having an ROC AUC of at least 0.809, and more preferably wherein the panel of one or more CpGs comprises at least the CpGs denoted by [[CG]];
263. one or more CpGs within DMR 263 as defined by SEQ ID NO: 5263 denoted by CG, preferably wherein the assay is characterised as having an ROC AUC of at least 0.809, and more preferably wherein the panel of one or more CpGs comprises at least the CpGs denoted by [[CG]];
264. one or more CpGs within DMR 264 as defined by SEQ ID NO: 5264 denoted by CG, preferably wherein the assay is characterised as having an ROC AUC of at least 0.808, and more preferably wherein the panel of one or more CpGs comprises at least the CpGs denoted by [[CG]];
265. one or more CpGs within DMR 265 as defined by SEQ ID NO: 5265 denoted by CG, preferably wherein the assay is characterised as having an ROC AUC of at least 0.808, and more preferably wherein the panel of one or more CpGs comprises at least the CpGs denoted by [[CG]];
266. one or more CpGs within DMR 266 as defined by SEQ ID NO: 5266 denoted by CG, preferably wherein the assay is characterised as having an ROC AUC of at least 0.808, and more preferably wherein the panel of one or more CpGs comprises at least the CpGs denoted by [[CG]];
267. one or more CpGs within DMR 267 as defined by SEQ ID NO: 5267 denoted by CG, preferably wherein the assay is characterised as having an ROC AUC of at least 0.808, and more preferably wherein the panel of one or more CpGs comprises at least the CpGs denoted by [[CG]];
268. one or more CpGs within DMR 268 as defined by SEQ ID NO: 5268 denoted by CG, preferably wherein the assay is characterised as having an ROC AUC of at least 0.808, and more preferably wherein the panel of one or more CpGs comprises at least the CpGs denoted by [[CG]];
269. one or more CpGs within DMR 269 as defined by SEQ ID NO: 5269 denoted by CG, preferably wherein the assay is characterised as having an ROC AUC of at least 0.808, and more preferably wherein the panel of one or more CpGs comprises at least the CpGs denoted by [[CG]];
270. one or more CpGs within DMR 270 as defined by SEQ ID NO: 5270 denoted by CG, preferably wherein the assay is characterised as having an ROC AUC of at least 0.808, and more preferably wherein the panel of one or more CpGs comprises at least the CpGs denoted by [[CG]];
271. one or more CpGs within DMR 271 as defined by SEQ ID NO: 5271 denoted by CG, preferably wherein the assay is characterised as having an ROC AUC of at least 0.808, and more preferably wherein the panel of one or more CpGs comprises at least the CpGs denoted by [[CG]]; 2
272. one or more CpGs within DMR 272 as defined by SEQ ID NO: 5272 denoted by CG, preferably wherein the assay is characterised as having an ROC AUC of at least 0.808, and more preferably wherein the panel of one or more CpGs comprises at least the CpGs denoted by [[CG]];
273. one or more CpGs within DMR 273 as defined by SEQ ID NO: 5273 denoted by CG, preferably wherein the assay is characterised as having an ROC AUC of at least 0.808, and more preferably wherein the panel of one or more CpGs comprises at least the CpGs denoted by [[CG]];
274. one or more CpGs within DMR 274 as defined by SEQ ID NO: 5274 denoted by CG, preferably wherein the assay is characterised as having an ROC AUC of at least 0.807, and more preferably wherein the panel of one or more CpGs comprises at least the CpGs denoted by [[CG]];
275. one or more CpGs within DMR 275 as defined by SEQ ID NO: 5275 denoted by CG, preferably wherein the assay is characterised as having an ROC AUC of at least 0.807, and more preferably wherein the panel of one or more CpGs comprises at least the CpGs denoted by [[CG]];
276. one or more CpGs within DMR 276 as defined by SEQ ID NO: 5276 denoted by CG, preferably wherein the assay is characterised as having an ROC AUC of at least 0.807, and more preferably wherein the panel of one or more CpGs comprises at least the CpGs denoted by [[CG]];
277. one or more CpGs within DMR 277 as defined by SEQ ID NO: 5277 denoted by CG, preferably wherein the assay is characterised as having an ROC AUC of at least 0.807, and more preferably wherein the panel of one or more CpGs comprises at least the CpGs denoted by [[CG]];
278. one or more CpGs within DMR 278 as defined by SEQ ID NO: 5278 denoted by CG, preferably wherein the assay is characterised as having an ROC AUC of at least 0.807, and more preferably wherein the panel of one or more CpGs comprises at least the CpGs denoted by [[CG]];
279. one or more CpGs within DMR 279 as defined by SEQ ID NO: 5279 denoted by CG, preferably wherein the assay is characterised as having an ROC AUC of at least 0.807, and more preferably wherein the panel of one or more CpGs comprises at least the CpGs denoted by [[CG]];
280. one or more CpGs within DMR 280 as defined by SEQ ID NO: 5280 denoted by CG, preferably wherein the assay is characterised as having an ROC AUC of at least 0.807, and more preferably wherein the panel of one or more CpGs comprises at least the CpGs denoted by [[CG]];
281. one or more CpGs within DMR 281 as defined by SEQ ID NO: 5281 denoted by CG, preferably wherein the assay is characterised as having an ROC AUC of at least 0.807, and more preferably wherein the panel of one or more CpGs comprises at least the CpGs denoted by [[CG]];
282. one or more CpGs within DMR 282 as defined by SEQ ID NO: 5282 denoted by CG, preferably wherein the assay is characterised as having an ROC AUC of at least 0.807, and more preferably wherein the panel of one or more CpGs comprises at least the CpGs denoted by [[CG]];
283. one or more CpGs within DMR 283 as defined by SEQ ID NO: 5283 denoted by CG, preferably wherein the assay is characterised as having an ROC AUC of at least 0.807, and more preferably wherein the panel of one or more CpGs comprises at least the CpGs denoted by [[CG]];
284. one or more CpGs within DMR 284 as defined by SEQ ID NO: 5284 denoted by CG, preferably wherein the assay is characterised as having an ROC AUC of at least 0.807, and more preferably wherein the panel of one or more CpGs comprises at least the CpGs denoted by [[CG]];
285. one or more CpGs within DMR 285 as defined by SEQ ID NO: 5285 denoted by CG, preferably wherein the assay is characterised as having an ROC AUC of at least 0.806, and more preferably wherein the panel of one or more CpGs comprises at least the CpGs denoted by [[CG]];
286. one or more CpGs within DMR 286 as defined by SEQ ID NO: 5286 denoted by CG, preferably wherein the assay is characterised as having an ROC AUC of at least 0.806, and more preferably wherein the panel of one or more CpGs comprises at least the CpGs denoted by [[CG]];
287. one or more CpGs within DMR 287 as defined by SEQ ID NO: 5287 denoted by CG, preferably wherein the assay is characterised as having an ROC AUC of at least 0.806, and more preferably wherein the panel of one or more CpGs comprises at least the CpGs denoted by [[CG]];
288. one or more CpGs within DMR 288 as defined by SEQ ID NO: 5288 denoted by CG, preferably wherein the assay is characterised as having an ROC AUC of at least 0.806, and more preferably wherein the panel of one or more CpGs comprises at least the CpGs denoted by [[CG]];
289. one or more CpGs within DMR 289 as defined by SEQ ID NO: 5289 denoted by CG, preferably wherein the assay is characterised as having an ROC AUC of at least 0.806, and more preferably wherein the panel of one or more CpGs comprises at least the CpGs denoted by [[CG]];
290. one or more CpGs within DMR 290 as defined by SEQ ID NO: 5290 denoted by CG, preferably wherein the assay is characterised as having an ROC AUC of at least 0.806, and more preferably wherein the panel of one or more CpGs comprises at least the CpGs denoted by [[CG]];
291. one or more CpGs within DMR 291 as defined by SEQ ID NO: 5291 denoted by CG, preferably wherein the assay is characterised as having an ROC AUC of at least 0.806, and more preferably wherein the panel of one or more CpGs comprises at least the CpGs denoted by [[CG]];
292. one or more CpGs within DMR 292 as defined by SEQ ID NO: 5292 denoted by CG, preferably wherein the assay is characterised as having an ROC AUC of at least 0.805, and more preferably wherein the panel of one or more CpGs comprises at least the CpGs denoted by [[CG]];
293. one or more CpGs within DMR 293 as defined by SEQ ID NO: 5293 denoted by CG, preferably wherein the assay is characterised as having an ROC AUC of at least 0.805, and more preferably wherein the panel of one or more CpGs comprises at least the CpGs denoted by [[CG]];
294. one or more CpGs within DMR 294 as defined by SEQ ID NO: 5294 denoted by CG, preferably wherein the assay is characterised as having an ROC AUC of at least 0.805, and more preferably wherein the panel of one or more CpGs comprises at least the CpGs denoted by [[CG]];
295. one or more CpGs within DMR 295 as defined by SEQ ID NO: 5295 denoted by CG, preferably wherein the assay is characterised as having an ROC AUC of at least 0.805, and more preferably wherein the panel of one or more CpGs comprises at least the CpGs denoted by [[CG]];
296. one or more CpGs within DMR 296 as defined by SEQ ID NO: 5296 denoted by CG, preferably wherein the assay is characterised as having an ROC AUC of at least 0.805, and more preferably wherein the panel of one or more CpGs comprises at least the CpGs denoted by [[CG]];
297. one or more CpGs within DMR 297 as defined by SEQ ID NO: 5297 denoted by CG, preferably wherein the assay is characterised as having an ROC AUC of at least 0.805, and more preferably wherein the panel of one or more CpGs comprises at least the CpGs denoted by [[CG]];
298. one or more CpGs within DMR 298 as defined by SEQ ID NO: 5298 denoted by CG, preferably wherein the assay is characterised as having an ROC AUC of at least 0.805, and more preferably wherein the panel of one or more CpGs comprises at least the CpGs denoted by [[CG]];
299. one or more CpGs within DMR 299 as defined by SEQ ID NO: 5299 denoted by CG, preferably wherein the assay is characterised as having an ROC AUC of at least 0.805, and more preferably wherein the panel of one or more CpGs comprises at least the CpGs denoted by [[CG]];
300. one or more CpGs within DMR 300 as defined by SEQ ID NO: 5300 denoted by CG, preferably wherein the assay is characterised as having an ROC AUC of at least 0.805, and more preferably wherein the panel of one or more CpGs comprises at least the CpGs denoted by [[CG]];
301. one or more CpGs within DMR 301 as defined by SEQ ID NO: 5301 denoted by CG, preferably wherein the assay is characterised as having an ROC AUC of at least 0.805, and more preferably wherein the panel of one or more CpGs comprises at least the CpGs denoted by [[CG]];
302. one or more CpGs within DMR 302 as defined by SEQ ID NO: 5302 denoted by CG, preferably wherein the assay is characterised as having an ROC AUC of at least 0.805, and more preferably wherein the panel of one or more CpGs comprises at least the CpGs denoted by [[CG]];
303. one or more CpGs within DMR 303 as defined by SEQ ID NO: 5303 denoted by CG, preferably wherein the assay is characterised as having an ROC AUC of at least 0.805, and more preferably wherein the panel of one or more CpGs comprises at least the CpGs denoted by [[CG]];
304. one or more CpGs within DMR 304 as defined by SEQ ID NO: 5304 denoted by CG, preferably wherein the assay is characterised as having an ROC AUC of at least 0.805, and more preferably wherein the panel of one or more CpGs comprises at least the CpGs denoted by [[CG]];
305. one or more CpGs within DMR 305 as defined by SEQ ID NO: 5305 denoted by CG, preferably wherein the assay is characterised as having an ROC AUC of at least 0.804, and more preferably wherein the panel of one or more CpGs comprises at least the CpGs denoted by [[CG]];
306. one or more CpGs within DMR 306 as defined by SEQ ID NO: 5306 denoted by CG, preferably wherein the assay is characterised as having an ROC AUC of at least 0.804, and more preferably wherein the panel of one or more CpGs comprises at least the CpGs denoted by [[CG]];
307. one or more CpGs within DMR 307 as defined by SEQ ID NO: 5307 denoted by CG, preferably wherein the assay is characterised as having an ROC AUC of at least 0.804, and more preferably wherein the panel of one or more CpGs comprises at least the CpGs denoted by [[CG]];
308. one or more CpGs within DMR 308 as defined by SEQ ID NO: 5308 denoted by CG, preferably wherein the assay is characterised as having an ROC AUC of at least 0.804, and more preferably wherein the panel of one or more CpGs comprises at least the CpGs denoted by [[CG]];
309. one or more CpGs within DMR 309 as defined by SEQ ID NO: 5309 denoted by CG, preferably wherein the assay is characterised as having an ROC AUC of at least 0.804, and more preferably wherein the panel of one or more CpGs comprises at least the CpGs denoted by [[CG]];
310. one or more CpGs within DMR 310 as defined by SEQ ID NO: 5310 denoted by CG, preferably wherein the assay is characterised as having an ROC AUC of at least 0.804, and more preferably wherein the panel of one or more CpGs comprises at least the CpGs denoted by [[CG]];
311. one or more CpGs within DMR 311 as defined by SEQ ID NO: 5311 denoted by CG, preferably wherein the assay is characterised as having an ROC AUC of at least 0.804, and more preferably wherein the panel of one or more CpGs comprises at least the CpGs denoted by [[CG]];
312. one or more CpGs within DMR 312 as defined by SEQ ID NO: 5312 denoted by CG, preferably wherein the assay is characterised as having an ROC AUC of at least 0.804, and more preferably wherein the panel of one or more CpGs comprises at least the CpGs denoted by [[CG]];
313. one or more CpGs within DMR 313 as defined by SEQ ID NO: 5313 denoted by CG, preferably wherein the assay is characterised as having an ROC AUC of at least 0.803, and more preferably wherein the panel of one or more CpGs comprises at least the CpGs denoted by [[CG]];
314. one or more CpGs within DMR 314 as defined by SEQ ID NO: 5314 denoted by CG, preferably wherein the assay is characterised as having an ROC AUC of at least 0.803, and more preferably wherein the panel of one or more CpGs comprises at least the CpGs denoted by [[CG]];
315. one or more CpGs within DMR 315 as defined by SEQ ID NO: 5315 denoted by CG, preferably wherein the assay is characterised as having an ROC AUC of at least 0.803, and more preferably wherein the panel of one or more CpGs comprises at least the CpGs denoted by [[CG]];
316. one or more CpGs within DMR 316 as defined by SEQ ID NO: 5316 denoted by CG, preferably wherein the assay is characterised as having an ROC AUC of at least 0.803, and more preferably wherein the panel of one or more CpGs comprises at least the CpGs denoted by [[CG]];
317. one or more CpGs within DMR 317 as defined by SEQ ID NO: 5317 denoted by CG, preferably wherein the assay is characterised as having an ROC AUC of at least 0.803, and more preferably wherein the panel of one or more CpGs comprises at least the CpGs denoted by [[CG]];
318. one or more CpGs within DMR 318 as defined by SEQ ID NO: 5318 denoted by CG, preferably wherein the assay is characterised as having an ROC AUC of at least 0.803, and more preferably wherein the panel of one or more CpGs comprises at least the CpGs denoted by [[CG]];
319. one or more CpGs within DMR 319 as defined by SEQ ID NO: 5319 denoted by CG, preferably wherein the assay is characterised as having an ROC AUC of at least 0.803, and more preferably wherein the panel of one or more CpGs comprises at least the CpGs denoted by [[CG]];
320. one or more CpGs within DMR 320 as defined by SEQ ID NO: 5320 denoted by CG, preferably wherein the assay is characterised as having an ROC AUC of at least 0.803, and more preferably wherein the panel of one or more CpGs comprises at least the CpGs denoted by [[CG]];
321. one or more CpGs within DMR 321 as defined by SEQ ID NO: 5321 denoted by CG, preferably wherein the assay is characterised as having an ROC AUC of at least 0.803, and more preferably wherein the panel of one or more CpGs comprises at least the CpGs denoted by [[CG]];
322. one or more CpGs within DMR 322 as defined by SEQ ID NO: 5322 denoted by CG, preferably wherein the assay is characterised as having an ROC AUC of at least 0.803, and more preferably wherein the panel of one or more CpGs comprises at least the CpGs denoted by [[CG]];
323. one or more CpGs within DMR 323 as defined by SEQ ID NO: 5323 denoted by CG, preferably wherein the assay is characterised as having an ROC AUC of at least 0.803, and more preferably wherein the panel of one or more CpGs comprises at least the CpGs denoted by [[CG]];
324. one or more CpGs within DMR 324 as defined by SEQ ID NO: 5324 denoted by CG, preferably wherein the assay is characterised as having an ROC AUC of at least 0.803, and more preferably wherein the panel of one or more CpGs comprises at least the CpGs denoted by [[CG]];
325. one or more CpGs within DMR 325 as defined by SEQ ID NO: 5325 denoted by CG, preferably wherein the assay is characterised as having an ROC AUC of at least 0.802, and more preferably wherein the panel of one or more CpGs comprises at least the CpGs denoted by [[CG]];
326. one or more CpGs within DMR 326 as defined by SEQ ID NO: 5326 denoted by CG, preferably wherein the assay is characterised as having an ROC AUC of at least 0.802, and more preferably wherein the panel of one or more CpGs comprises at least the CpGs denoted by [[CG]];
327. one or more CpGs within DMR 327 as defined by SEQ ID NO: 5327 denoted by CG, preferably wherein the assay is characterised as having an ROC AUC of at least 0.802, and more preferably wherein the panel of one or more CpGs comprises at least the CpGs denoted by [[CG]];
328. one or more CpGs within DMR 328 as defined by SEQ ID NO: 5328 denoted by CG, preferably wherein the assay is characterised as having an ROC AUC of at least 0.802, and more preferably wherein the panel of one or more CpGs comprises at least the CpGs denoted by [[CG]];
329. one or more CpGs within DMR 329 as defined by SEQ ID NO: 5329 denoted by CG, preferably wherein the assay is characterised as having an ROC AUC of at least 0.802, and more preferably wherein the panel of one or more CpGs comprises at least the CpGs denoted by [[CG]];
330. one or more CpGs within DMR 330 as defined by SEQ ID NO: 5330 denoted by CG, preferably wherein the assay is characterised as having an ROC AUC of at least 0.802, and more preferably wherein the panel of one or more CpGs comprises at least the CpGs denoted by [[CG]];
331. one or more CpGs within DMR 331 as defined by SEQ ID NO: 5331 denoted by CG, preferably wherein the assay is characterised as having an ROC AUC of at least 0.802, and more preferably wherein the panel of one or more CpGs comprises at least the CpGs denoted by [[CG]];
332. one or more CpGs within DMR 332 as defined by SEQ ID NO: 5332 denoted by CG, preferably wherein the assay is characterised as having an ROC AUC of at least 0.802, and more preferably wherein the panel of one or more CpGs comprises at least the CpGs denoted by [[CG]];
333. one or more CpGs within DMR 333 as defined by SEQ ID NO: 5333 denoted by CG, preferably wherein the assay is characterised as having an ROC AUC of at least 0.802, and more preferably wherein the panel of one or more CpGs comprises at least the CpGs denoted by [[CG]];
334. one or more CpGs within DMR 334 as defined by SEQ ID NO: 5334 denoted by CG, preferably wherein the assay is characterised as having an ROC AUC of at least 0.802, and more preferably wherein the panel of one or more CpGs comprises at least the CpGs denoted by [[CG]];
335. one or more CpGs within DMR 335 as defined by SEQ ID NO: 5335 denoted by CG, preferably wherein the assay is characterised as having an ROC AUC of at least 0.802, and more preferably wherein the panel of one or more CpGs comprises at least the CpGs denoted by [[CG]];
336. one or more CpGs within DMR 336 as defined by SEQ ID NO: 5336 denoted by CG, preferably wherein the assay is characterised as having an ROC AUC of at least 0.802, and more preferably wherein the panel of one or more CpGs comprises at least the CpGs denoted by [[CG]];
337. one or more CpGs within DMR 337 as defined by SEQ ID NO: 5337 denoted by CG, preferably wherein the assay is characterised as having an ROC AUC of at least 0.802, and more preferably wherein the panel of one or more CpGs comprises at least the CpGs denoted by [[CG]];
338. one or more CpGs within DMR 338 as defined by SEQ ID NO: 5338 denoted by CG, preferably wherein the assay is characterised as having an ROC AUC of at least 0.802, and more preferably wherein the panel of one or more CpGs comprises at least the CpGs denoted by [[CG]];
339. one or more CpGs within DMR 339 as defined by SEQ ID NO: 5339 denoted by CG, preferably wherein the assay is characterised as having an ROC AUC of at least 0.802, and more preferably wherein the panel of one or more CpGs comprises at least the CpGs denoted by [[CG]];
340. one or more CpGs within DMR 340 as defined by SEQ ID NO: 5340 denoted by CG, preferably wherein the assay is characterised as having an ROC AUC of at least 0.802, and more preferably wherein the panel of one or more CpGs comprises at least the CpGs denoted by [[CG]];
341. one or more CpGs within DMR 341 as defined by SEQ ID NO: 5341 denoted by CG, preferably wherein the assay is characterised as having an ROC AUC of at least 0.802, and more preferably wherein the panel of one or more CpGs comprises at least the CpGs denoted by [[CG]];
342. one or more CpGs within DMR 342 as defined by SEQ ID NO: 5342 denoted by CG, preferably wherein the assay is characterised as having an ROC AUC of at least 0.802, and more preferably wherein the panel of one or more CpGs comprises at least the CpGs denoted by [[CG]];
343. one or more CpGs within DMR 343 as defined by SEQ ID NO: 5343 denoted by CG, preferably wherein the assay is characterised as having an ROC AUC of at least 0.802, and more preferably wherein the panel of one or more CpGs comprises at least the CpGs denoted by [[CG]];
344. one or more CpGs within DMR 344 as defined by SEQ ID NO: 5344 denoted by CG, preferably wherein the assay is characterised as having an ROC AUC of at least 0.801, and more preferably wherein the panel of one or more CpGs comprises at least the CpGs denoted by [[CG]];
345. one or more CpGs within DMR 345 as defined by SEQ ID NO: 5345 denoted by CG, preferably wherein the assay is characterised as having an ROC AUC of at least 0.801, and more preferably wherein the panel of one or more CpGs comprises at least the CpGs denoted by [[CG]];
346. one or more CpGs within DMR 346 as defined by SEQ ID NO: 5346 denoted by CG, preferably wherein the assay is characterised as having an ROC AUC of at least 0.801, and more preferably wherein the panel of one or more CpGs comprises at least the CpGs denoted by [[CG]];
347. one or more CpGs within DMR 347 as defined by SEQ ID NO: 5347 denoted by CG, preferably wherein the assay is characterised as having an ROC AUC of at least 0.801, and more preferably wherein the panel of one or more CpGs comprises at least the CpGs denoted by [[CG]];
348. one or more CpGs within DMR 348 as defined by SEQ ID NO: 5348 denoted by CG, preferably wherein the assay is characterised as having an ROC AUC of at least 0.801, and more preferably wherein the panel of one or more CpGs comprises at least the CpGs denoted by [[CG]];
349. one or more CpGs within DMR 349 as defined by SEQ ID NO: 5349 denoted by CG, preferably wherein the assay is characterised as having an ROC AUC of at least 0.801, and more preferably wherein the panel of one or more CpGs comprises at least the CpGs denoted by [[CG]];
350. one or more CpGs within DMR 350 as defined by SEQ ID NO: 5350 denoted by CG, preferably wherein the assay is characterised as having an ROC AUC of at least 0.801, and more preferably wherein the panel of one or more CpGs comprises at least the CpGs denoted by [[CG]];
351. one or more CpGs within DMR 351 as defined by SEQ ID NO: 5351 denoted by CG, preferably wherein the assay is characterised as having an ROC AUC of at least 0.801, and more preferably wherein the panel of one or more CpGs comprises at least the CpGs denoted by [[CG]];
352. one or more CpGs within DMR 352 as defined by SEQ ID NO: 5352 denoted by CG, preferably wherein the assay is characterised as having an ROC AUC of at least 0.801, and more preferably wherein the panel of one or more CpGs comprises at least the CpGs denoted by [[CG]];
353. one or more CpGs within DMR 353 as defined by SEQ ID NO: 5353 denoted by CG, preferably wherein the assay is characterised as having an ROC AUC of at least 0.801, and more preferably wherein the panel of one or more CpGs comprises at least the CpGs denoted by [[CG]];
354. one or more CpGs within DMR 354 as defined by SEQ ID NO: 5354 denoted by CG, preferably wherein the assay is characterised as having an ROC AUC of at least 0.801, and more preferably wherein the panel of one or more CpGs comprises at least the CpGs denoted by [[CG]];
355. one or more CpGs within DMR 355 as defined by SEQ ID NO: 5355 denoted by CG, preferably wherein the assay is characterised as having an ROC AUC of at least 0.801, and more preferably wherein the panel of one or more CpGs comprises at least the CpGs denoted by [[CG]];
356. one or more CpGs within DMR 356 as defined by SEQ ID NO: 5356 denoted by CG, preferably wherein the assay is characterised as having an ROC AUC of at least 0.801, and more preferably wherein the panel of one or more CpGs comprises at least the CpGs denoted by [[CG]];
357. one or more CpGs within DMR 357 as defined by SEQ ID NO: 5357 denoted by CG, preferably wherein the assay is characterised as having an ROC AUC of at least 0.801, and more preferably wherein the panel of one or more CpGs comprises at least the CpGs denoted by [[CG]];
358. one or more CpGs within DMR 358 as defined by SEQ ID NO: 5358 denoted by CG, preferably wherein the assay is characterised as having an ROC AUC of at least 0.801, and more preferably wherein the panel of one or more CpGs comprises at least the CpGs denoted by [[CG]];
359. one or more CpGs within DMR 359 as defined by SEQ ID NO: 5359 denoted by CG, preferably wherein the assay is characterised as having an ROC AUC of at least 0.801, and more preferably wherein the panel of one or more CpGs comprises at least the CpGs denoted by [[CG]];
360. one or more CpGs within DMR 360 as defined by SEQ ID NO: 5360 denoted by CG, preferably wherein the assay is characterised as having an ROC AUC of at least 0.801, and more preferably wherein the panel of one or more CpGs comprises at least the CpGs denoted by [[CG]];
361. one or more CpGs within DMR 361 as defined by SEQ ID NO: 5361 denoted by CG, preferably wherein the assay is characterised as having an ROC AUC of at least 0.801, and more preferably wherein the panel of one or more CpGs comprises at least the CpGs denoted by [[CG]];
362. one or more CpGs within DMR 362 as defined by SEQ ID NO: 5362 denoted by CG, preferably wherein the assay is characterised as having an ROC AUC of at least 0.801, and more preferably wherein the panel of one or more CpGs comprises at least the CpGs denoted by [[CG]];
363. one or more CpGs within DMR 363 as defined by SEQ ID NO: 5363 denoted by CG, preferably wherein the assay is characterised as having an ROC AUC of at least 0.801, and more preferably wherein the panel of one or more CpGs comprises at least the CpGs denoted by [[CG]];
364. one or more CpGs within DMR 364 as defined by SEQ ID NO: 5364 denoted by CG, preferably wherein the assay is characterised as having an ROC AUC of at least 0.801, and more preferably wherein the panel of one or more CpGs comprises at least the CpGs denoted by [[CG]];
365. one or more CpGs within DMR 365 as defined by SEQ ID NO: 5365 denoted by CG, preferably wherein the assay is characterised as having an ROC AUC of at least 0.801, and more preferably wherein the panel of one or more CpGs comprises at least the CpGs denoted by [[CG]];
366. one or more CpGs within DMR 366 as defined by SEQ ID NO: 5366 denoted by CG, preferably wherein the assay is characterised as having an ROC AUC of at least 0.801, and more preferably wherein the panel of one or more CpGs comprises at least the CpGs denoted by [[CG]];
367. one or more CpGs within DMR 367 as defined by SEQ ID NO: 5367 denoted by CG, preferably wherein the assay is characterised as having an ROC AUC of at least 0.801, and more preferably wherein the panel of one or more CpGs comprises at least the CpGs denoted by [[CG]];
368. one or more CpGs within DMR 368 as defined by SEQ ID NO: 5368 denoted by CG, preferably wherein the assay is characterised as having an ROC AUC of at least 0.801, and more preferably wherein the panel of one or more CpGs comprises at least the CpGs denoted by [[CG]];
369. one or more CpGs within DMR 369 as defined by SEQ ID NO: 5369 denoted by CG, preferably wherein the assay is characterised as having an ROC AUC of at least 0.801, and more preferably wherein the panel of one or more CpGs comprises at least the CpGs denoted by [[CG]];
370. one or more CpGs within DMR 370 as defined by SEQ ID NO: 5370 denoted by CG, preferably wherein the assay is characterised as having an ROC AUC of at least 0.801, and more preferably wherein the panel of one or more CpGs comprises at least the CpGs denoted by [[CG]];
371. one or more CpGs within DMR 371 as defined by SEQ ID NO: 5371 denoted by CG, preferably wherein the assay is characterised as having an ROC AUC of at least 0.801, and more preferably wherein the panel of one or more CpGs comprises at least the CpGs denoted by [[CG]];
372. one or more CpGs within DMR 372 as defined by SEQ ID NO: 5372 denoted by CG, preferably wherein the assay is characterised as having an ROC AUC of at least 0.801, and more preferably wherein the panel of one or more CpGs comprises at least the CpGs denoted by [[CG]];
373. one or more CpGs within DMR 373 as defined by SEQ ID NO: 5373 denoted by CG, preferably wherein the assay is characterised as having an ROC AUC of at least 0.801, and more preferably wherein the panel of one or more CpGs comprises at least the CpGs denoted by [[CG]];
374. one or more CpGs within DMR 374 as defined by SEQ ID NO: 5374 denoted by CG, preferably wherein the assay is characterised as having an ROC AUC of at least 0.801, and more preferably wherein the panel of one or more CpGs comprises at least the CpGs denoted by [[CG]];
375. one or more CpGs within DMR 375 as defined by SEQ ID NO: 5375 denoted by CG, preferably wherein the assay is characterised as having an ROC AUC of at least 0.800, and more preferably wherein the panel of one or more CpGs comprises at least the CpGs denoted by [[CG]];
376. one or more CpGs within DMR 376 as defined by SEQ ID NO: 5376 denoted by CG, preferably wherein the assay is characterised as having an ROC AUC of at least 0.800, and more preferably wherein the panel of one or more CpGs comprises at least the CpGs denoted by [[CG]];
377. one or more CpGs within DMR 377 as defined by SEQ ID NO: 5377 denoted by CG, preferably wherein the assay is characterised as having an ROC AUC of at least 0.800, and more preferably wherein the panel of one or more CpGs comprises at least the CpGs denoted by [[CG]];
378. one or more CpGs within DMR 378 as defined by SEQ ID NO: 5378 denoted by CG, preferably wherein the assay is characterised as having an ROC AUC of at least 0.800, and more preferably wherein the panel of one or more CpGs comprises at least the CpGs denoted by [[CG]];
379. one or more CpGs within DMR 379 as defined by SEQ ID NO: 5379 denoted by CG, preferably wherein the assay is characterised as having an ROC AUC of at least 0.800, and more preferably wherein the panel of one or more CpGs comprises at least the CpGs denoted by [[CG]];
380. one or more CpGs within DMR 380 as defined by SEQ ID NO: 5380 denoted by CG, preferably wherein the assay is characterised as having an ROC AUC of at least 0.800, and more preferably wherein the panel of one or more CpGs comprises at least the CpGs denoted by [[CG]];
381. one or more CpGs within DMR 381 as defined by SEQ ID NO: 5381 denoted by CG, preferably wherein the assay is characterised as having an ROC AUC of at least 0.800, and more preferably wherein the panel of one or more CpGs comprises at least the CpGs denoted by [[CG]];
382. one or more CpGs within DMR 382 as defined by SEQ ID NO: 5382 denoted by CG, preferably wherein the assay is characterised as having an ROC AUC of at least 0.800, and more preferably wherein the panel of one or more CpGs comprises at least the CpGs denoted by [[CG]];
383. one or more CpGs within DMR 383 as defined by SEQ ID NO: 5383 denoted by CG, preferably wherein the assay is characterised as having an ROC AUC of at least 0.800, and more preferably wherein the panel of one or more CpGs comprises at least the CpGs denoted by [[CG]];
384. one or more CpGs within DMR 384 as defined by SEQ ID NO: 5384 denoted by CG, preferably wherein the assay is characterised as having an ROC AUC of at least 0.800, and more preferably wherein the panel of one or more CpGs comprises at least the CpGs denoted by [[CG]];
385. one or more CpGs within DMR 385 as defined by SEQ ID NO: 5385 denoted by CG, preferably wherein the assay is characterised as having an ROC AUC of at least 0.800, and more preferably wherein the panel of one or more CpGs comprises at least the CpGs denoted by [[CG]];
386. one or more CpGs within DMR 386 as defined by SEQ ID NO: 5386 denoted by CG, preferably wherein the assay is characterised as having an ROC AUC of at least 0.800, and more preferably wherein the panel of one or more CpGs comprises at least the CpGs denoted by [[CG]];
387. one or more CpGs within DMR 387 as defined by SEQ ID NO: 5387 denoted by CG, preferably wherein the assay is characterised as having an ROC AUC of at least 0.800, and more preferably wherein the panel of one or more CpGs comprises at least the CpGs denoted by [[CG]];
388. one or more CpGs within DMR 388 as defined by SEQ ID NO: 5388 denoted by CG, preferably wherein the assay is characterised as having an ROC AUC of at least 0.800, and more preferably wherein the panel of one or more CpGs comprises at least the CpGs denoted by [[CG]];
389. one or more CpGs within DMR 389 as defined by SEQ ID NO: 5389 denoted by CG, preferably wherein the assay is characterised as having an ROC AUC of at least 0.800, and more preferably wherein the panel of one or more CpGs comprises at least the CpGs denoted by [[CG]];
390. one or more CpGs within DMR 390 as defined by SEQ ID NO: 5390 denoted by CG, preferably wherein the assay is characterised as having an ROC AUC of at least 0.800, and more preferably wherein the panel of one or more CpGs comprises at least the CpGs denoted by [[CG]];
391. one or more CpGs within DMR 391 as defined by SEQ ID NO: 5391 denoted by CG, preferably wherein the assay is characterised as having an ROC AUC of at least 0.860, and more preferably wherein the panel of one or more CpGs comprises at least the CpGs denoted by [[CG]];
392. one or more CpGs within DMR 392 as defined by SEQ ID NO: 5392 denoted by CG, preferably wherein the assay is characterised as having an ROC AUC of at least 0.858, and more preferably wherein the panel of one or more CpGs comprises at least the CpGs denoted by [[CG]];
393. one or more CpGs within DMR 393 as defined by SEQ ID NO: 5393 denoted by CG, preferably wherein the assay is characterised as having an ROC AUC of at least 0.855, and more preferably wherein the panel of one or more CpGs comprises at least the CpGs denoted by [[CG]];
394. one or more CpGs within DMR 394 as defined by SEQ ID NO: 5394 denoted by CG, preferably wherein the assay is characterised as having an ROC AUC of at least 0.851, and more preferably wherein the panel of one or more CpGs comprises at least the CpGs denoted by [[CG]];
395. one or more CpGs within DMR 395 as defined by SEQ ID NO: 5395 denoted by CG, preferably wherein the assay is characterised as having an ROC AUC of at least 0.845, and more preferably wherein the panel of one or more CpGs comprises at least the CpGs denoted by [[CG]];
396. one or more CpGs within DMR 396 as defined by SEQ ID NO: 5396 denoted by CG, preferably wherein the assay is characterised as having an ROC AUC of at least 0.839, and more preferably wherein the panel of one or more CpGs comprises at least the CpGs denoted by [[CG]];
397. one or more CpGs within DMR 397 as defined by SEQ ID NO: 5397 denoted by CG, preferably wherein the assay is characterised as having an ROC AUC of at least 0.839, and more preferably wherein the panel of one or more CpGs comprises at least the CpGs denoted by [[CG]];
398. one or more CpGs within DMR 398 as defined by SEQ ID NO: 5398 denoted by CG, preferably wherein the assay is characterised as having an ROC AUC of at least 0.837, and more preferably wherein the panel of one or more CpGs comprises at least the CpGs denoted by [[CG]];
399. one or more CpGs within DMR 399 as defined by SEQ ID NO: 5399 denoted by CG, preferably wherein the assay is characterised as having an ROC AUC of at least 0.835, and more preferably wherein the panel of one or more CpGs comprises at least the CpGs denoted by [[CG]];
400. one or more CpGs within DMR 400 as defined by SEQ ID NO: 5400 denoted by CG, preferably wherein the assay is characterised as having an ROC AUC of at least 0.834, and more preferably wherein the panel of one or more CpGs comprises at least the CpGs denoted by [[CG]];
401. one or more CpGs within DMR 401 as defined by SEQ ID NO: 5401 denoted by CG, preferably wherein the assay is characterised as having an ROC AUC of at least 0.827, and more preferably wherein the panel of one or more CpGs comprises at least the CpGs denoted by [[CG]];
402. one or more CpGs within DMR 402 as defined by SEQ ID NO: 5402 denoted by CG, preferably wherein the assay is characterised as having an ROC AUC of at least 0.825, and more preferably wherein the panel of one or more CpGs comprises at least the CpGs denoted by [[CG]];
403. one or more CpGs within DMR 403 as defined by SEQ ID NO: 5403 denoted by CG, preferably wherein the assay is characterised as having an ROC AUC of at least 0.825, and more preferably wherein the panel of one or more CpGs comprises at least the CpGs denoted by [[CG]];
404. one or more CpGs within DMR 404 as defined by SEQ ID NO: 5404 denoted by CG, preferably wherein the assay is characterised as having an ROC AUC of at least 0.814, and more preferably wherein the panel of one or more CpGs comprises at least the CpGs denoted by [[CG]];
405. one or more CpGs within DMR 405 as defined by SEQ ID NO: 5405 denoted by CG, preferably wherein the assay is characterised as having an ROC AUC of at least 0.811, and more preferably wherein the panel of one or more CpGs comprises at least the CpGs denoted by [[CG]];
406. one or more CpGs within DMR 406 as defined by SEQ ID NO: 5406 denoted by CG, preferably wherein the assay is characterised as having an ROC AUC of at least 0.806, and more preferably wherein the panel of one or more CpGs comprises at least the CpGs denoted by [[CG]];
407. one or more CpGs within DMR 407 as defined by SEQ ID NO: 5407 denoted by CG, preferably wherein the assay is characterised as having an ROC AUC of at least 0.804, and more preferably wherein the panel of one or more CpGs comprises at least the CpGs denoted by [[CG]];
408. one or more CpGs within DMR 408 as defined by SEQ ID NO: 5408 denoted by CG, preferably wherein the assay is characterised as having an ROC AUC of at least 0.801, and more preferably wherein the panel of one or more CpGs comprises at least the CpGs denoted by [[CG]];
409. one or more CpGs within DMR 409 as defined by SEQ ID NO: 5409 denoted by CG, preferably wherein the assay is characterised as having an ROC AUC of at least 0.799, and more preferably wherein the panel of one or more CpGs comprises at least the CpGs denoted by [[CG]];
410. one or more CpGs within DMR 410 as defined by SEQ ID NO: 5410 denoted by CG, preferably wherein the assay is characterised as having an ROC AUC of at least 0.798, and more preferably wherein the panel of one or more CpGs comprises at least the CpGs denoted by [[CG]];
411. one or more CpGs within DMR 411 as defined by SEQ ID NO: 5411 denoted by CG, preferably wherein the assay is characterised as having an ROC AUC of at least 0.784, and more preferably wherein the panel of one or more CpGs comprises at least the CpGs denoted by [[CG]];
412. one or more CpGs within DMR 412 as defined by SEQ ID NO: 5412 denoted by CG, preferably wherein the assay is characterised as having an ROC AUC of at least 0.78, and more preferably wherein the panel of one or more CpGs comprises at least the CpGs denoted by [[CG]];
413. one or more CpGs within DMR 413 as defined by SEQ ID NO: 5413 denoted by CG, preferably wherein the assay is characterised as having an ROC AUC of at least 0.771, and more preferably wherein the panel of one or more CpGs comprises at least the CpGs denoted by [[CG]];
414. one or more CpGs within DMR 414 as defined by SEQ ID NO: 5414 denoted by CG, preferably wherein the assay is characterised as having an ROC AUC of at least 0.768, and more preferably wherein the panel of one or more CpGs comprises at least the CpGs denoted by [[CG]];
415. one or more CpGs within DMR 415 as defined by SEQ ID NO: 5415 denoted by CG, preferably wherein the assay is characterised as having an ROC AUC of at least 0.766, and more preferably wherein the panel of one or more CpGs comprises at least the CpGs denoted by [[CG]];
416. one or more CpGs within DMR 416 as defined by SEQ ID NO: 5416 denoted by CG, preferably wherein the assay is characterised as having an ROC AUC of at least 0.761, and more preferably wherein the panel of one or more CpGs comprises at least the CpGs denoted by [[CG]];
417. one or more CpGs within DMR 417 as defined by SEQ ID NO: 5417 denoted by CG, preferably wherein the assay is characterised as having an ROC AUC of at least 0.747, and more preferably wherein the panel of one or more CpGs comprises at least the CpGs denoted by [[CG]]; or
418. one or more CpGs within DMR 418 as defined by SEQ ID NO: 5418 denoted by CG, preferably wherein the assay is characterised as having an ROC AUC of at least 0.739, and more preferably wherein the panel of one or more CpGs comprises at least the CpGs denoted by [[CG]],
   preferably wherein the assay comprises determining methylation β-values for each CpG in the panel of one or more CpGs, more preferably wherein the cancer is cervical cancer.

In any one of the assays described herein, the step of determining the methylation status of the one or more CpGs in the panel may comprise or may additionally comprise determining the methylation status of each CpG within one or more of the sequences identified by SEQ ID NOs 5703 to 5786.

In any one of the assays described herein, the step of determining the methylation status of the one or more CpGs in the panel may preferably comprise or may preferably additionally comprise determining the methylation status of each CpG within one or more of the sequences identified by SEQ ID NOs 5703, 5731, 5759, 5723, 5751, 5779, 5713, 5741, 5769, 5706, 5734, 5762, 5705, 5733 and 5761. More preferably, in any one of the assays described herein, the step of determining the methylation status of the one or more CpGs in the panel may comprise or may additionally comprise determining the methylation status of each CpG within all of the sequences identified by SEQ ID NOs 5703, 5731, 5759, 5723, 5751, 5779, 5713, 5741, 5769, 5706, 5734, 5762, 5705, 5733 and 5761.

In any one of the assays described herein, the step of determining the methylation status of the one or more CpGs in the panel may preferably comprise or may preferably additionally comprise determining the methylation status of each CpG within one or more of the sequences identified by SEQ ID NOs 5703, 5731, 5759, 5713, 5741, 5769, 5706, 5734, and 5762. More preferably, in any one of the assays described herein, the step of determining the methylation status of the one or more CpGs in the panel may comprise or may additionally comprise determining the methylation status of each CpG within all of the sequences identified by SEQ ID NOs SEQ ID NOs 5703, 5731, 5759, 5713, 5741, 5769, 5706, 5734, and 5762.

The invention also provides a variety of assays, each comprising any 1, 2, 3, 4, 5, 6, 7, 8, 9, 10 or more (or any range derivable therein) of a variety of steps and in no particular order, including methods of the following: measuring in a sample; analyzing a sample; assessing a sample; evaluating a sample; measuring nucleic acids in a sample; assessing nucleic acids in a sample; detecting nucleic acids in a sample; measuring methylation in nucleic acids in a sample; analyzing nucleic acids in a sample; assessing nucleic acids in a sample; measuring methylation at one or more CpG dinucleotides in a sample; detecting methylation at one or more CpG dinucleotides in a sample; assaying methylation at one or more CpG dinucleotides in a sample; assessing methylation at one or more CpG dinucleotides in a sample; measuring a methylation status in a sample; assaying a methylation status in a sample; detecting methylation status in a sample; determining methylation status in a sample; identifying methylation status in a sample; measuring one or more DNA methylation markers in a sample; assessing one or more DNA methylation markers in a sample; detecting one or more DNA methylation markers in a sample; measuring the presence of methylation at one or more markers in a sample; detecting the presence of methylation at one or more markers in a sample; assessing the presence of methylation at one or more markers in a sample; assaying the presence of one of more markers in a sample; measuring one or more DNA methylation markers in a sample but excluding the measuring of one or more other DNA methylation markers in the sample; assessing one or more DNA methylation markers in a sample but excluding the assessing of one or more other DNA methylation markers in the sample; analyzing one or more DNA methylation markers in a sample but excluding the analyzing of one or more other DNA methylation markers in the sample; detecting one or more DNA methylation markers in a sample but excluding the detecting of one or more other DNA methylation markers in the sample; measuring methylation status in nucleic acids from a sample from tissue from an individual other than tissue from the individual suspected of, or at risk for, being cancerous; detecting methylation status in nucleic acids from a sample from tissue from an individual other than tissue from the individual suspected of, or at risk for, being cancerous; analyzing methylation status in nucleic acids from a sample from tissue from an individual other than tissue from the individual suspected of, or at risk for, being cancerous; assessing methylation status in nucleic acids from a sample from tissue from an individual other than tissue from the individual suspected of, or at risk for, being cancerous; measuring methylation at one or more CpG dinucleotides in a sample but excluding the measuring of methylation at one or more CpG dinucleotides in the sample; assessing methylation at one or more CpG dinucleotides in a sample but excluding the assessing of methylation at one or more CpG dinucleotides in the sample; analyzing methylation at one or more CpG dinucleotides in a sample but excluding the analyzing of methylation at one or more CpG dinucleotides in the sample; detecting methylation at one or more CpG dinucleotides in a sample but excluding the detecting of methylation at one or more CpG dinucleotides in the sample; measuring methylation at one or more CpG dinucleotides in nucleic acids from a sample from tissue from an individual other than tissue from the individual suspected of, or at risk for, being cancerous; detecting methylation at one or more CpG dinucleotides in nucleic acids from a sample from tissue from an individual other than tissue from the individual suspected of, or at risk for, being cancerous; analyzing methylation at one or more CpG dinucleotides in nucleic acids from a sample from tissue from an individual other than tissue from the individual suspected of, or at risk for, being cancerous; assessing methylation at one or more CpG dinucleotides in nucleic acids from a sample from tissue from an individual other than tissue from the individual suspected of, or at risk for, being cancerous; treating an individual for cancer when the individual has been determined to have a methylation status at one or more methylation markers; treating an individual for cancer when the individual has been determined to have methylation at one or more CpG dinucleotides;
wherein any of the aforementioned methods, or any other methods encompassed by the disclosure, may comprise any one or more of the following method steps:
measuring methylation status, wherein the measuring identifies the methylation status of one or markers from nucleic acids in a sample;
measuring methylation status, wherein the measuring identifies the presence of one or more markers from nucleic acids in a sample;
measuring the presence of one or more methylation markers from a sample;
providing DNA from a sample;
providing nucleic acids from a sample;
determining whether one or more methylation markers from nucleic acids from a sample are methylated;
measuring whether one or more methylation markers from nucleic acids from a sample are methylated;
performing a sequencing step on nucleic acids from a sample;
determining a sequence of nucleic acids from a sample;
performing bisulphite conversion on one or more markers;
performing bisulphite conversion on one or more CpG dinucleotides;
hybridizing DNA to an array comprising probes capable of determining methylated versus non-methylated markers;
hybridizing DNA to an array comprising probes capable of determining methylated versus non-methylated CpG dinucleotides;
hybridizing DNA to an array comprising probes capable of discriminating between methylated and non-methylated markers;
hybridizing DNA to an array comprising probes capable of discriminating between methylated and non-methylated CpG dinucleotides;
performing an amplification step on sequence from nucleic acids from a sample;
performing an amplification step on sequence from nucleic acids using methylation-specific primers;
performing amplification of sequence comprising one or more regions suspected of having methylation or in need of determination of a methylation status;
performing PCR on sequence comprising one or more regions suspected of having methylation or in need of determination of a methylation status;
performing a capturing step;
performing a binding step;
performing a purification step;
performing a capturing step comprising binding of polynucleotides comprising one or more methylation markers to binding molecules specific to the one or more methylation markers and collecting complexes thereof;
stratifying the grade of a cancer;
determining the risk of cancer;
determining the risk of recurrence of cancer;
obtaining a sample from an individual;
obtaining DNA from a sample from an individual;
administering a treatment to an individual;
providing DNA from a sample;
determining whether one or more methylation markers from a panel of methylation markers comprises a specific sequence; and/or
obtaining data that identifies whether each one of a group of methylation markers from a panel comprises a specific sequence.

Moreover, in some aspects of the invention, an individual who is administered a therapy or treatment has been subjected to any of the methods and steps described herein.

Described herein are assays that utilise a statistically robust panel of one or more CpGs whose methylation status can be determined to provide a reliable prediction of the presence or development of CIN3 and/or cancer in an individual. By determining the methylation status of each CpG within the panel of one or more CpGs, a cancer index value may be derived thus enabling stratification of individuals according to their risk of developing CIN3 and/r cancer or of having cancer, particularly cervical and/or endometrial cancer, with statistically robust sensitivity and specificity. The skilled person would understand that the methylation status of each CpG within a panel of one or more CpGs can be determined by any suitable means in order to thereby derive the cancer index value. Any one method, or a combination of methods, may be used to determine the methylation status of each CpG within a panel of one or more CpGs.

Various exemplary methods for determining the methylation status of each CpG within a panel of one or more CpGs are described herein. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

### Cancer Index Values as Diagnostic and Risk Assessment Tools

In any of the assays described herein, the assessment of the presence, absence or development of CIN3 and/or cancer, particularly cervical or endometrial cancer, most preferably cervical cancer, in an individual is based on the cancer index value of the individual at the time of testing.

As explained herein, using panels of the specific CpGs disclosed herein, cancer index values can be established which correspond with CIN3 and/or cancer negative samples, because they are based on values derived from individuals known to be CIN3 and/or cancer negative. Similarly, using panels of the specific CpGs disclosed herein, cancer index values can be established which correspond with CIN3 and/or cancer positive samples from individuals known to be CIN3 and/or cancer positive. A user can then apply these cancer index values to assess the presence, absence or development of CIN3 and/or cancer, particularly cervical or endometrial cancer, most preferably cervical cancer, in any test individual whose CIN3 and/or cancer status is required to be tested. As also explained herein, the assays of the invention are capable of being performed with a high degree of statistical accuracy.

As explained herein, the described assays particularly relate to the assessment of the presence, absence or development of cervical cancer and/or endometrial cancer, particularly cervical cancer.

A skilled person will readily appreciate that a cancer index value provides a value that indicates a “likelihood” or “risk” or “prediction” of any of the assays of the invention correctly assessing the presence, absence or development of CIN3 and/or cancer, particularly cervical or endometrial cancer, most preferably cervical cancer, in an individual. This because the assessment is based upon a correlation between DNA methylation profiles of tissue samples and individual disease status. Nevertheless, as demonstrated by data set out in the Examples and elsewhere herein, the assays of the invention provide such correlations with high statistical accuracy, thus providing the skilled person with a high degree of confidence that the cancer index value which is determined for any test individual whose cancer status is required to be tested will provide an accurate correlation with actual disease status for the individual.

In the context of the present invention, “likelihood”, “risk” and “prediction” may be used synonymously with each other.

Any references herein to sequences, genomic sequences and/or genomic coordinates are derived based upon Homo sapiens (human) genome assembly GRCh37 (hg19). The skilled person would understand variations in the nucleotide sequences of any given sequence, particularly DMRs 1 to 418, may exist due to sequencing errors and/or variation between individuals.

The assay of the invention represents a ‘prediction’ because any cancer index value (WID-CIN-Index) derived in accordance with the invention is unlikely to be capable of diagnosing every individual as having or not having cancer with 100% specificity and 100% sensitivity. Rather, depending on the cancer index cutpoint threshold applied by the user for positively predicting the presence of cancer in an individual, the false positive and false negative rate will vary. In other words, the inventors have discovered that the assays of the invention can achieve variable levels of sensitivity and specificity for predicting the presence, absence or development of CIN3 and/or cancer, particularly cervical or endometrial cancer, most preferably cervical cancer, as defined by receiver operating characteristics, depending on the cancer index cutpoint threshold chosen and applied by the user. Such sensitivity and specificity can be seen from the data disclosed herein to be achievable at high proportions, demonstrating accurate and statistically-significant discriminatory capability.

Similarly, cancer index values which have been pre-determined to correlate with specific cancer phenotypes, such as the presence or absence of cancer, have been defined with a high level of statistical accuracy as explained further herein.

Assessing the ‘development’ of cancer in the context of the invention may refer to assessing whether an individual is likely or unlikely to develop cancer. Cells from sampled these tissues/anatomical sites can act as a surrogate for cervical and/or endometrial cells that may transform to cancer. Assessing the development of cancer in accordance with the assays of the invention may refer to assessing an increased or decreased likelihood of CIN3 and/or cancer development, particularly cervical cancer and endometrial cancer, preferably cervical cancer. Assessing the development of cancer in accordance with the assays of the invention may refer to assessing progression or worsening of a pre-existing cancer lesion in an individual. Assessment of the development of cancer in accordance with the assays of the invention may refer to predicting the likelihood of recurrence of cancer.

In any of the assays described herein, the step of assessing the presence or development of CIN3 and/or cancer in an individual based on a cancer index value may involve the application of a threshold value. Threshold values can provide a risk-based indication of an individual's CIN3 and/or cancer status, whether that is CIN3 and/or cancer positive, or CIN3 and/or cancer negative. Threshold values can also provide a means for identifying whether the cancer index value is intermediate between a CIN3 and/or cancer positive value and a CIN3 and/or cancer negative value. As explained herein, the cancer index value may be dynamic and subject to change depending upon genetic and/or environmental factors. Accordingly, the cancer index value may provide a means for assessing and monitoring cancer development. Cancer index values may therefore indicate at least a low risk or a high risk that the individual has a CIN3 and/or cancer positive status or has a status that is indicative of the development of CIN3 and/or cancer. If the cancer index value of an individual is determined by the assays of the invention at two or more time points, an increase or decrease in the individual's cancer index value may indicate an increased or decreased risk of the individual having or developing CIN3 and/or cancer, particularly cervical and/or endometrial cancer, most preferably cervical cancer.

Throughout the disclosure herein the terms “threshold value”, “cutpoint”, and “cutpoint threshold” are to be considered synonymous and interchangeable.

As explained further herein any assay of the invention is an assay for assessing the presence, absence or development of CIN3 and/or cancer, particularly cervical or endometrial cancer, most preferably cervical cancer, in an individual. The types of cancer are set out further herein. As explained further herein, the assays of the invention provide means for assessing whether an individual is at risk of having or developing CIN3 and/or cancer based on specific cutpoint thresholds. Such risk assessments can be provided with a high degree of confidence based on the statistical parameters which characterise the assay. Thus in any of the assays described herein involving cancer index cutpoint thresholds, the cutpoint threshold may be used for risk assessment purposes. Equally, in any of the assays described herein involving cancer index cutpoint thresholds, the cutpoint threshold value may be used to specify whether or not an individual has CIN3 and/or cancer as a pure diagnostic test. Again, such diagnostic tests can be provided with a high degree of confidence based on the statistical parameters which characterise the assay. Accordingly, in any assay described herein which specifies that a cancer index value for the individual is a specific value or more, or is “about” a specific value or more, the individual may be assessed as having cancer. In any assay described herein which specifies that a cancer index value for the individual is less than a specific value, or is less than “about” a specific value, the individual may be assessed as not having cancer. The term “about” is to be understood as providing a range of +/−5% of the value.

Accordingly, any assay of the invention is an assay for assessing the presence, absence or development of CIN3 and/or cancer, particularly cervical or endometrial cancer, most preferably cervical cancer, in an individual, the assay comprising:
a. providing a sample which has been taken from the individual, the sample comprising a population of DNA molecules;
b. determining in the population of DNA molecules in the sample the methylation status of a panel of:
   (i) one or more CpGs selected from a panel of CpGs identified in SEQ ID NOs 1 to 5000 wherein the CpGs are identified at nucleotide positions 61 to 62; and/or
   (ii) one or more CpGs selected from within a panel of one or more Differentially Methylated Regions (DMRs) defined by SEQ ID NOs 5001 to 5418, wherein the CpGs are denoted by CG;
c. deriving a cancer index value based on the methylation status of the one or more CpGs in the panel; and
d. assessing the presence, absence or development of CIN3 and/or cancer, particularly cervical or endometrial cancer, most preferably cervical cancer, in the individual based on the cancer index value;
wherein the assay is characterised as having an area under the curve (AUC) of 0.60 or more as determined by receiver operating characteristics (ROC).

Any of the assays of the invention are particularly for assessing the presence or absence of CIN3 and/or cancer in an individual.

Such an assay may be performed in accordance with any of the methods disclosed and defined herein.

As explained further herein, any assay of the invention for assessing the presence, absence or development of CIN3 and/or cancer, particularly cervical or endometrial cancer, most preferably cervical cancer, in an individual may alternatively be referred to as an assay for stratifying an individual in accordance with their CIN3 and/or cancer status.

Accordingly, any assay of the invention is an assay for stratifying an individual for the presence, absence or development of CIN3 and/or cancer, particularly cervical or endometrial cancer, most preferably cervical cancer, in an individual, the assay comprising:
a. providing a sample which has been taken from the individual, the sample comprising a population of DNA molecules;
b. determining in the population of DNA molecules in the sample the methylation status of a panel of:
   (i) one or more CpGs selected from a panel of CpGs identified in SEQ ID NOs 1 to 5000 wherein the CpGs are identified at nucleotide positions 61 to 62; and/or
   (ii) one or more CpGs selected from within a panel of one or more Differentially Methylated Regions (DMRs) defined by SEQ ID NOs 5001 to 5418, wherein the CpGs are denoted by CG;
c. deriving a cancer index value based on the methylation status of the one or more CpGs in the panel; and
d. stratifying the individual for the presence, absence or development of CIN3 and/or cancer, particularly cervical or endometrial cancer, most preferably cervical cancer, based on the cancer index value;
wherein the assay is characterised as having an area under the curve (AUC) of 0.60 or more as determined by receiver operating characteristics (ROC).

Such an assay may be performed in accordance with any of the methods disclosed and defined herein.

Accordingly, any assay of the invention is an assay for stratifying an individual for CIN3 and/or cancer, the assay comprising:
a. providing a sample which has been taken from the individual, the sample comprising a population of DNA molecules;
b. determining in the population of DNA molecules in the sample the methylation status of a panel of:
   (i) one or more CpGs selected from a panel of CpGs identified in SEQ ID NOs 1 to 5000 wherein the CpGs are identified at nucleotide positions 61 to 62; and/or
   (ii) one or more CpGs selected from within a panel of one or more Differentially Methylated Regions (DMRs) defined by SEQ ID NOs 5001 to 5418, wherein the CpGs are denoted by CG;
c. deriving a cancer index value based on the methylation status of the one or more CpGs in the panel; and
d. stratifying the individual for cancer based on the cancer index value;
wherein the assay is characterised as having an area under the curve (AUC) of 0.60 or more as determined by receiver operating characteristics (ROC).

Such an assay may be performed in accordance with any of the methods disclosed and defined herein.

The cancer index value may be derived by any suitable means. Preferably, the cancer index value may be derived by assessing the methylation status of the panel of:
(i) one or more CpGs selected from a panel of CpGs identified in SEQ ID NOs 1 to 5000 wherein the CpGs are identified at nucleotide positions 61 to 62; and/or
(ii) one or more CpGs selected from within a panel of one or more Differentially Methylated Regions (DMRs) defined by SEQ ID NOs 5001 to 5418, wherein the CpGs are denoted by CG,

in a sample provided from an individual. The methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

In any of the assays described herein the step of determining the methylation status of each CpG in the panel of one or more CpGs may comprise bisulphite converting the DNA.

The step of determining in the population of DNA molecules in the sample the methylation status of a panel of one or more CpGs may comprises determining a β value of each CpG. Deriving the cancer index value may involve providing a methylation β-value data set comprising the methylation β-values for each CpG in the panel of one or more CpGs. Additionally, or alternatively, the step of determining in the population of DNA molecules in the sample the methylation status of a panel of one or more CpGs may comprises determining a percent methylated reference value for each of the panel of one or more CpGs. Optionally deriving the cancer index value may also involve estimating the fraction of contaminating DNA within the DNA provided from a sample.

DNA may be DNA originating from a particular source organism, tissue or cell type. Preferably the contaminating DNA originates from one or more different cell types to one or more cell types of interest. A cell type of interest may particularly be an epithelial cell. In some aspects of the invention, it may be preferable to estimate the fraction of contaminating DNA after the step of providing a sample which has been take from an individual. The assays described herein may optionally involve estimating a contaminating DNA fraction within DNA in the sample by any suitable means. Preferably, the contaminating DNA fraction for the sample is estimated via any suitable bioinformatics analysis tool. A bioinformatics analysis tool that may be used to estimate a contaminating DNA fraction may be EpIDISH. As described herein, it may be desirable to estimate the fraction of contaminating DNA from the one or more cell types that are different to the one or more cell types of interest because the cancer index value used for predicting the presence or development of cancer in an individual may, in some instances, only be reliably derived from determining the methylation status of a set of CpGs from DNA of a particular cell type of interest. Particularly, methylation status beta-values may differ in the one or more cell types of interest within a sample relative to methylation status beta-values in contaminating DNA from different cell types within the same sample. Thus, the derived cancer index value may in some instances have a decreased predictive power without estimating and controlling for the contaminating DNA fraction within the DNA provided from the sample. In assays of the invention that involve estimating the fraction of contaminating DNA and accordingly controlling for said contaminating DNA, it is preferable to estimate an immune cell DNA fraction within the DNA provided from the sample. In particular assays of the invention, wherein the individual has an immune cell contamination of over 50% (i.e. wherein more than 50% of the DNA in the sample is deemed to be derived from immune cells), the assay may preferably involve controlling for the immune cell contamination by deriving the cancer index, in accordance with the invention, solely from the DNA molecules derived from epithelial cells.

Any of the assays described herein comprising a step of deriving a cancer index value based on the methylation status of the one or more CpGs in the panel may further comprise applying an algorithm to the methylation beta-value dataset to obtain the cancer index value. Preferably, in any of the assays described herein, the step of deriving the cancer index value based on the methylation status of the panel of CpGs comprises providing a methylation beta-value data set comprising the methylation beta-values for each CpG in the panel and applying an algorithm to the methylation beta-value data set to obtain the cancer index value.

In any of the assays described herein, the step of deriving the cancer index value based on the methylation status of the one or more CpGs in the panel may comprise:
a. providing a methylation β-value data set comprising the methylation β-values for each CpG in the panel;
b. providing a mathematical model capable of generating the cancer index from the methylation β-value data set; and
c. applying the mathematical model to the methylation β-value data set, thereby generating the cancer index.

In any of the assays described herein, the cancer index value may be calculated by any suitable mathematical model such as an algorithm or formula. Preferably, the cancer index value is termed Women's risk Identification for Cervical Cancer Index (WID-CIN-index) and wherein the mathematical model which is applied to the methylation β-value data set to generate the cancer index is calculated by an algorithm according to the following formula:

$$WID - CIN - index = {\sum }_{i = 1}^{n} \frac{μ - {w}_{i} {β}_{i}}{σ}$$

wherein:
1. β₁, . . . , βₙ are methylation beta-values (between 0 and 1);
2. w₁, . . . , w₅₀₀₀ are real valued coefficients;
3. μ and σ are real valued parameters used to scale the index; and
4. n refers to the number of CpGs in the set of test CpGs;
preferably wherein the cancer is endometrial cancer.

In any of the assays described herein, the WID-CIN-index algorithm applies real valued coefficients inferred by initially training on a dataset (this dataset in the exemplary embodiments of the invention described in the Examples consisted of 165 CIN3+ cases and 202 human papillomavirus positive (HPV+) controls) to fit a ridge classifier using the R package glmnet with a mixing parameter value of alpha=0 (ridge penalty) and binomial response type. Ten-fold cross-validation was used internally by the cv.glmnet function in order to determine the optimal value of the regularisation parameter lambda. The beta values from n CpGs for individual ν, β₁^{ν}, . . . , βₙ^{ν}, are used as inputs to the ridge classifier. The coefficients w₁, . . . , wₙ are obtained from the fitted model. The following quantity was computed for each individual ν in the training set:

$${x}_{v} = {\sum }_{i = 1}^{n} {w}_{i} {β}_{i}^{v}$$

Any suitable real valued coefficients may be applied to the WID-CIN-Index in any of the assays described herein.

The value of the parameters μ and σ are given by the mean and standard deviation of x_{ν} in the training dataset respectively.

Thus, any suitable μ and σ real valued parameters may be applied to the WID-CIN-index in any of the assays described herein. Any suitable training data set may be applied to the assays described herein in order to infer real valued parameters and coefficients that can subsequently be applied to the WID-CIN-index formula according to the present invention. Exemplary ways of utilising a training dataset in accordance with the present invention are further described in the ‘Statistical analyses for classifier development’ section of the Materials and Methods section of the Examples.

Exemplary μ and σ real valued parameters are provided in Table 2 for CpG subsets identified in SEQ ID NOs 1 to 5000. These real valued parameters may be applied to any of the assays described herein wherein the real parameters correspond to any one of the sets of CpGs identified in SEQ ID NOs 1 to 5000 and set out in the left hand column of Table 3.

TABLE 3
Exemplary μ and σ real valued parameters are provided in
Table 3 for CpG subsets identified in SEQ ID NOs 1 to 5000

| SEQ ID | | |
|---|---|---|
| NOs: | mu | sigma |
| 1-100 | 2.740972 | 5.738187 |
| 1-500 | 7.58544 | 7.762485 |
| 1-1000 | 5.731128 | 8.397056 |
| 1-1500 | 5.609192 | 9.469183 |
| 1-2000 | 5.498524 | 10.06744 |
| 1-2500 | 4.994593 | 9.862255 |
| 1-3000 | 5.11682 | 9.974974 |
| 1-3500 | 3.809614 | 8.180184 |
| 1-4000 | 4.352256 | 10.55814 |
| 1-4500 | 4.416423 | 9.848581 |
| 1-5000 | 4.268773 | 10.38904 |

Exemplary w₁, . . . , wₙ real value coefficients are provided below for the CpGs identified at positions 61 to 62 in SEQ ID NOs 1 to 5000. These real value coefficients may be applied to any of the assays described herein wherein the real parameters correspond to any one of the sets of CpGs identified in SEQ ID NOs 1 to 5000 wherein the 5000 real value coefficients below in turn correspond to the CpGs in turn identified at nucleotide positions 61 to 62 in SEQ ID NOs 1 to 5000. Accordingly, the listed coefficients are presented below in numerical order corresponding respectively to the CpGs identified in SEQ ID NOs 1 to 5000. Thus the first number below corresponds to SEQ ID NO 1, the second number corresponds to SEQ ID NO 2 etc. The exemplary real value coefficients are as follows:
−1.4775, −1.12307, −1.06088, 1.02708, −0.96865, −0.87277, 0.85797, 0.85533, 0.84542, −0.84206, −0.8321, −0.81011, −0.79471, 0.77851, −0.77179, −0.75927, 0.75082, −0.73863, −0.7318, 0.72738, 0.72644, −0.7134, 0.71318, 0.70257, −0.6891, −0.6883, 0.67779, −0.67584, 0.66994, −0.667, −0.6634, −0.6558, 0.65126, −0.64549, 0.64461, −0.64282, 0.63756, −0.63593, −0.63406, −0.63239, −0.62702, −0.62655, 0.62338, 0.61844, −0.61788, −0.61317, 0.60845, −0.60205, −0.59891, 0.59795, 0.5958, 0.59007, 0.58909, −0.58345, 0.58343, −0.58072, −0.57796, −0.56978, 0.56904, −0.56875, 0.56635, −0.56237, −0.55676, 0.55659, −0.55655, −0.55407, −0.55107, −0.55091, −0.55045, 0.55042, 0.54961, 0.54723, 0.54665, −0.54636, −0.54566, 0.54097, −0.53854, −0.53758, −0.53165, −0.52949, −0.52598, 0.52439, −0.52218, −0.52083, −0.51658, 0.51647, −0.51358, 0.51072, 0.51028, 0.50981, −0.50841, 0.50286, −0.50242, −0.49984, −0.49701, −0.49605, −0.49543, −0.49485, −0.49472, −0.49469, −0.49309, −0.48824, −0.48715, 0.48711, −0.48433, 0.48313, 0.48294, −0.48258, −0.48251, −0.48197, −0.48011, 0.47981, −0.47821, 0.476, −0.47481, −0.47374, 0.4733, −0.4716, 0.47091, 0.47028, 0.46854, 0.46782, −0.4677, −0.46738, 0.46548, −0.46218, −0.46142, 0.46072, 0.46064, −0.45973, 0.45913, −0.45882, −0.45805, 0.45732, 0.45726, 0.45588, 0.45555, −0.45529, 0.45168, 0.45058, 0.45051, 0.44934, −0.44906, 0.44896, −0.44841, 0.44837, −0.44764, 0.44714, −0.44601, 0.44583, 0.44535, 0.44155, −0.44058, 0.44035, 0.43869, 0.43775, −0.43757, −0.43745, 0.43701, 0.43676, 0.43551, 0.43259, 0.43171, 0.43165, −0.43081, −0.42966, 0.42941, −0.42855, −0.4283, −0.42811, −0.42726, −0.42653, 0.42533, −0.42497, −0.42227, 0.42197, 0.42112, 0.42065, −0.42017, 0.41908, −0.41877, −0.41752, 0.41744, −0.41699, −0.41692, −0.41629, −0.41507, 0.41444, −0.41419, 0.41388, 0.41252, −0.41247, −0.41095, 0.40819, 0.40749, 0.40686, 0.4068, 0.40677, −0.40377, −0.40374, −0.40252, −0.40054, 0.39925, −0.39864, −0.39699, 0.39649, −0.39624, −0.39583, −0.3953, −0.39496, −0.39482, 0.39471, −0.39437, −0.39338, −0.39292, 0.39274, 0.39259, 0.3915, −0.39145, 0.39104, −0.39039, −0.39002, −0.3891, 0.38898, −0.38861, −0.38804, 0.38715, −0.38646, 0.38602, −0.38594, 0.38586, −0.3853, −0.38497, 0.38362, −0.38354, −0.383, −0.38281, −0.38096, −0.38045, −0.38024, 0.38002, −0.37994, −0.37937, −0.37767, 0.37711, −0.3768, −0.37629, 0.37623, 0.37534, −0.37525, 0.3749, −0.37383, 0.37336, 0.37282, 0.37192, 0.37191, −0.37097, 0.37079, −0.37068, 0.37047, 0.37041, 0.37028, −0.37016, 0.36947, −0.36861, −0.36807, 0.36779, −0.36706, −0.36682, −0.36616, 0.36615, −0.36479, −0.36473, 0.36451, 0.36409, −0.3629, 0.36281, 0.36178, −0.36176, 0.36069, 0.36024, 0.35967, −0.35955, −0.35946, −0.35936, 0.35719, −0.35713, 0.35628, −0.35409, 0.35395, 0.35376, −0.35299, −0.35255, 0.3502, 0.34911, 0.34869, 0.34786, −0.34731, −0.3471, −0.34694, 0.34675, 0.34558, 0.34527, 0.34508, 0.34378, 0.34336, −0.34305, 0.34299, 0.34214, −0.34168, 0.34143, 0.34138, 0.34034, 0.34026, −0.34019, 0.33913, 0.33878, 0.33822, −0.33782, −0.33778, −0.33715, −0.33713, −0.33665, −0.33625, −0.33623, 0.33616, −0.33561, 0.33559, 0.33525, −0.33489, 0.33477, −0.33312, 0.33209, −0.33134, −0.3305, −0.33018, 0.32949, −0.32908, 0.32858, 0.32858, 0.32795, 0.3279, 0.32741, −0.32719, −0.32718, 0.32703, 0.32691, −0.32668, −0.32659, 0.32609, −0.32464, −0.3245, 0.32405, 0.32346, −0.32291, −0.32267, −0.32219, −0.32198, −0.32197, −0.32185, −0.32129, 0.32038, −0.31978, −0.31957, 0.31914, 0.31908, −0.31904, −0.31885, −0.31876, −0.31864, 0.31804, −0.31803, 0.31776, −0.31706, −0.31689, −0.31626, −0.31614, −0.31607, 0.31574, −0.31553, −0.31525, 0.31518, −0.31511, −0.31467, 0.31413, −0.31391, −0.31389, −0.3138, −0.3134, 0.31327, −0.31314, 0.313, 0.31292, −0.31269, −0.3126, 0.3125, 0.31178, −0.31143, 0.31132, 0.31075, −0.3105, −0.30969, 0.30928, 0.30863, −0.30829, 0.30777, 0.30744, −0.30664, −0.30618, −0.30603, −0.30603, −0.30568, −0.30566, 0.30496, 0.30485, 0.30471, −0.30431, −0.30418, −0.30363, −0.30298, −0.30289, −0.30286, 0.30273, −0.30251, 0.30162, −0.30149, 0.30131, 0.30105, −0.30075, −0.30067, 0.30019, 0.29954, 0.29932, −0.29929, −0.29923, 0.29811, −0.298, 0.29791, −0.29759, 0.29747, 0.29739, 0.2973, −0.29718, 0.29686, −0.29589, 0.29583, −0.29553, −0.29544, 0.29505, −0.29498, −0.29483, −0.29472, −0.29457, −0.29421, 0.29396, −0.29395, 0.29355, 0.29323, 0.29296, −0.29269, −0.29268, −0.29173, −0.29138, 0.29128, 0.29032, 0.29016, 0.28977, −0.28877, −0.28853, 0.28826, 0.28777, 0.28759, −0.28734, 0.28707, −0.28678, 0.28627, 0.28603, 0.28595, −0.28568, 0.2855, 0.28543, −0.28538, −0.28476, −0.28467, −0.28462, −0.28439, −0.28433, 0.28392, −0.28383, −0.28378, 0.28364, 0.28321, −0.283, 0.28291, 0.28291, −0.2828, −0.28264, 0.28248, −0.28148, −0.28142, 0.28123, 0.28046, −0.27976, −0.27975, 0.27962, 0.27924, 0.27877, 0.27816, 0.27801, 0.27732, −0.27667, −0.27651, 0.27644, −0.27633, 0.27626, −0.27616, 0.27613, 0.27601, −0.27591, −0.27582, −0.27574, 0.2757, −0.27471, −0.27457, 0.27455, 0.2742, −0.27409, 0.2739, −0.27333, −0.2733, −0.2727, −0.27248, −0.27213, 0.27197, −0.27158, −0.27131, 0.27111, −0.27047, −0.27002, −0.26996, 0.26988, −0.26977, 0.26976, −0.26969, −0.26927, −0.26918, 0.26906, −0.26896, 0.26894, 0.26893, −0.26874, 0.26838, −0.26817, 0.26792, −0.26742, −0.26739, −0.26736, 0.26734, −0.26717, −0.26691, −0.2669, −0.26685, −0.26616, −0.26615, 0.2657, 0.26568, −0.26561, 0.26525, 0.26501, 0.2649, −0.26476, −0.26474, −0.2644, −0.26409, 0.26401, −0.26401, −0.26387, 0.26305, −0.26301, 0.26295, −0.26295, −0.26106, 0.26057, −0.26037, 0.26031, −0.26019, 0.26018, 0.26007, 0.26005, −0.25972, −0.25953, 0.25942, 0.25928, −0.25917, 0.25907, −0.25881, −0.25871, −0.25653, −0.25617, −0.2558, −0.25502, −0.25482, −0.25463, −0.25424, −0.25354, 0.25344, −0.25343, −0.25337, −0.25329, −0.25307, 0.25303, 0.2528, 0.25258, −0.25252, 0.25248, −0.25246, −0.25235, −0.25207, 0.25206, −0.25181, 0.2515, −0.25133, 0.25108, −0.25085, −0.25074, −0.25066, −0.24994, −0.24965, −0.2493, −0.24908, −0.24901, −0.24886, −0.2488, −0.24878, −0.24871, 0.24844, 0.24828, 0.24825, 0.24819, 0.24764, 0.24751, −0.2471, 0.24671, −0.2467, −0.24664, 0.24663, −0.24645, 0.24635, −0.2462, −0.24612, −0.24593, −0.24507, −0.24467, −0.24462, −0.24458, 0.24454, 0.24454, −0.24433, 0.2441, −0.24404, 0.24394, −0.2436, −0.24355, −0.24312, 0.24305, −0.24301, 0.24291, 0.24272, −0.2423, −0.24215, −0.24205, 0.24175, 0.2416, −0.24159, −0.24157, −0.24151, −0.24151, 0.24126, −0.24104, 0.24101, −0.24076, −0.2407, 0.2406, −0.24056, −0.24049, −0.24048, −0.24047, −0.24041, 0.24018, 0.23993, −0.23982, −0.23978, 0.23968, −0.23941, −0.23941, −0.23935, 0.23905, 0.239, 0.2387, 0.2387, 0.23857, −0.23848, 0.23837, −0.23814, −0.23711, 0.23669, −0.23649, −0.236, 0.23587, −0.23577, −0.23564, −0.23535, −0.23501, −0.23488, 0.23486, 0.23477, 0.23433, −0.23431, −0.2338, −0.23367, −0.23342, 0.23336, −0.23315, −0.23311, −0.23301, 0.23296, −0.23286, 0.2323, 0.23167, −0.23131, 0.23117, 0.23117, 0.23099, −0.23075, 0.23073, −0.23058, −0.2302, 0.23008, −0.2295, −0.22933, 0.22907, −0.22902, 0.22885, 0.22856, −0.22804, 0.22789, 0.22765, −0.22761, 0.22754, 0.22747, −0.22731, −0.22692, −0.22688, −0.22685, 0.22677, −0.22671, 0.22661, −0.22657, −0.22643, 0.22624, 0.22615, −0.22437, 0.22435, 0.22427, −0.22418, −0.22359, −0.22353, −0.22348, −0.22312, −0.22307, −0.22305, 0.22292, 0.22248, −0.22216, 0.22214, 0.22207, −0.22201, 0.22193, −0.22186, −0.22183, −0.22181, 0.22158, −0.22134, 0.22115, 0.22113, −0.22105, 0.22093, 0.22071, −0.22054, 0.22022, −0.21973, 0.21951, 0.21945, −0.21912, 0.21884, −0.21884, −0.21869, −0.21846, 0.21839, 0.21838, −0.21764, −0.21759, −0.21726, −0.21702, −0.21698, 0.21684, 0.21676, 0.21594, 0.2156, −0.21528, 0.21465, −0.21463, −0.21458, −0.21452, 0.21433, 0.21425, −0.21421, 0.21409, −0.21409, 0.21388, −0.21381, 0.21374, 0.21326, −0.21295, −0.21248, 0.21247, −0.21243, 0.21226, 0.21199, −0.21155, −0.21126, 0.21111, −0.21104, −0.21089, 0.21085, −0.21033, −0.21017, 0.21004, −0.21002, 0.20997, 0.2098, −0.20947, −0.20929, 0.2091, −0.20878, −0.20868, −0.20867, 0.20858, −0.20816, −0.20789, 0.20778, −0.20747, −0.20729, 0.20688, 0.20663, −0.20637, −0.20613, −0.20563, 0.20525, −0.20497, −0.20466, 0.2046, −0.20423, 0.20422, −0.20401, −0.2039, −0.20376, 0.20364, −0.20352, −0.20349, 0.20342, −0.20341, 0.2033, 0.20289, −0.2028, 0.20266, −0.20257, 0.20238, 0.20232, −0.20211, −0.2019, −0.20182, −0.2017, 0.20091, 0.20084, −0.20071, −0.20059, −0.20024, −0.20023, 0.19954, 0.19928, 0.19908, −0.19888, 0.19771, −0.19746, −0.19718, −0.19695, −0.19692, −0.19676, −0.19663, 0.1966, −0.19632, −0.19611, 0.19603, −0.19554, 0.19543, −0.19445, −0.19445, −0.1944, 0.19424, −0.19413, −0.19411, 0.1941, 0.19409, −0.19403, 0.19373, −0.19344, −0.19336, 0.19324, −0.19321, −0.19301, −0.19288, −0.19287, −0.19283, 0.19262, 0.19256, 0.19249, 0.19219, 0.19209, 0.19195, −0.19155, −0.19122, 0.19104, 0.19088, 0.19087, −0.19079, −0.19051, −0.19039, 0.1903, −0.18996, 0.18985, 0.1898, 0.18977, −0.18972, −0.18971, −0.18946, −0.18945, −0.18939, 0.18906, 0.1889, 0.18871, −0.18867, −0.18864, −0.18824, −0.18807, −0.188, −0.18783, −0.18779, 0.18776, −0.18773, 0.18754, 0.18747, −0.18732, 0.18711, −0.18658, 0.18644, 0.18638, −0.18579, 0.18577, −0.18571, 0.18546, −0.18532, −0.1852, −0.18517, −0.18508, 0.18498, −0.18467, 0.18462, 0.1846, 0.18445, 0.18434, −0.1843, 0.18424, 0.18375, 0.18365, 0.18336, −0.18335, 0.18329, −0.18308, −0.18296, 0.18293, −0.18289, −0.18279, −0.18267, −0.18262, 0.1826, −0.18245, −0.18245, −0.18243, 0.1824, 0.18225, −0.18195, 0.18193, −0.18185, −0.18172, 0.18161, 0.18117, −0.18014, −0.1799, 0.17985, −0.17947, −0.17873, 0.17873, 0.17868, 0.17794, −0.17776, 0.17774, −0.1777, 0.17726, −0.17712, 0.17706, −0.17704, −0.17687, −0.17685, 0.17674, −0.17674, −0.17673, 0.17667, −0.17667, −0.17666, 0.17666, −0.17663, −0.17661, 0.17659, −0.17655, −0.17655, 0.17652, 0.17637, −0.1763, −0.17629, 0.17627, −0.17624, −0.17592, −0.17561, 0.17561, 0.1752, 0.17513, −0.17512, 0.17511, 0.17501, −0.17478, −0.17465, 0.17455, −0.17441, 0.17439, −0.17436, −0.17429, 0.17426, −0.17416, −0.1741, 0.17375, −0.17371, 0.17337, −0.1733, −0.17329, 0.1731, 0.17309, −0.17305, −0.17273, −0.17265, −0.17254, −0.17232, −0.17217, 0.17203, −0.17175, −0.17167, 0.17164, 0.17148, −0.17126, −0.17123, 0.17077, −0.17067, −0.17066, 0.1706, −0.17041, −0.16993, 0.16992, −0.16982, −0.16971, −0.16965, 0.16959, −0.16955, 0.16939, 0.16936, −0.16892, −0.16873, 0.1683, −0.16823, −0.16819, −0.16806, 0.168, −0.16754, −0.16752, 0.16742, 0.16727, −0.16684, 0.16678, 0.16653, 0.16633, −0.16621, 0.16598, −0.16597, 0.16566, −0.16532, 0.16526, −0.16524, −0.16521, 0.16471, 0.16461, −0.16445, 0.16442, 0.16439, −0.16438, 0.16426, −0.16403, −0.16367, −0.16362, −0.16358, 0.16306, −0.16297, −0.16295, 0.16277, −0.16277, 0.16277, 0.16276, −0.16267, −0.16257, −0.16195, −0.16195, −0.16193, −0.16192, −0.1618, 0.16172, −0.16146, 0.16106, 0.16104, 0.1606, −0.16035, −0.16, 0.15994, 0.1597, −0.15967, −0.15952, −0.15952, −0.1595, −0.15938, −0.15933, −0.15929, −0.1591, −0.15902, −0.159, 0.15898, −0.15897, 0.1588, −0.15854, 0.1585, 0.15845, −0.15842, 0.15839, 0.15837, −0.1583, −0.15829, −0.15823, −0.15778, 0.1576, −0.1573, −0.1573, 0.15707, 0.15698, 0.15694, 0.15655, −0.15606, 0.15553, −0.1555, 0.15544, −0.15539, −0.15538, −0.15534, 0.15479, −0.15474, 0.15454, −0.15447, −0.15439, 0.1543, −0.15429, −0.1542, −0.1539, 0.1538, 0.15361, 0.15353, 0.1535, −0.15339, −0.15333, −0.15295, −0.15292, −0.1529, −0.15268, 0.15266, −0.15264, −0.1524, −0.15234, 0.15226, −0.15219, −0.15204, 0.15193, −0.15192, 0.1518, 0.15175, −0.15175, −0.15151, −0.15149, −0.15118, −0.15116, −0.15115, −0.1507, −0.15064, −0.15052, −0.15037, 0.15019, −0.15014, 0.14998, −0.14984, 0.1497, −0.14969, −0.14963, −0.14952, −0.14952, 0.14945, −0.14923, −0.14906, −0.14903, 0.14873, −0.14867, 0.14861, 0.14858, 0.14803, 0.14799, −0.14784, −0.14771, −0.14768, −0.14762, −0.14739, 0.14722, −0.14704, −0.14636, 0.14606, −0.14602, −0.14573, 0.1454, −0.14525, −0.14519, −0.14519, 0.14513, −0.14491, 0.14463, −0.14462, 0.14453, 0.14436, −0.14429, 0.14423, 0.14409, 0.14395, 0.14386, 0.14382, −0.14382, −0.14351, 0.14329, 0.14319, 0.14313, −0.14308, 0.143, 0.14298, −0.14294, −0.1427, −0.14268, −0.14205, −0.14202, 0.14197, 0.14192, 0.14171, 0.14163, 0.14122, −0.14117, −0.14108, −0.14067, −0.1405, −0.14033, −0.13999, −0.13996, −0.13987, −0.13984, −0.13982, −0.13982, 0.13965, −0.13951, 0.13951, 0.13938, −0.1393, 0.13922, −0.13921, −0.13911, 0.1385, −0.13848, 0.13845, 0.13832, −0.13826, 0.13817, −0.13791, −0.13753, 0.13743, −0.1374, 0.13692, 0.13689, 0.13686, −0.13684, 0.1367, −0.13669, −0.13665, −0.13663, −0.13655, 0.13648, −0.1363, 0.13623, 0.13619, −0.13598, 0.13582, −0.13582, 0.13578, 0.13565, 0.13553, −0.13551, 0.13548, −0.13541, −0.13538, −0.13531, 0.13526, 0.13517, −0.13512, −0.13511, −0.13505, 0.13493, −0.1349, −0.13473, 0.13465, −0.13461, −0.13441, −0.13436, −0.13431, −0.13422, −0.13385, −0.1338, 0.13353, −0.13351, −0.13349, −0.1334, −0.1333, −0.13329, −0.1332, −0.13313, −0.13273, 0.13265, −0.13264, 0.13258, −0.13244, −0.13229, 0.13221, 0.13218, −0.13217, 0.13215, −0.13206, −0.13206, 0.13189, 0.13189, −0.13169, −0.13163, 0.13154, 0.13151, −0.1315, 0.13138, −0.13127, −0.13123, 0.1311, −0.13087, −0.13076, −0.13073, 0.13072, 0.13063, −0.13061, −0.1306, −0.13056, −0.13055, 0.13046, −0.13043, 0.13036, 0.12999, −0.12984, −0.12971, −0.12969, −0.12966, 0.12965, −0.12963, −0.12954, −0.12934, 0.1293, 0.1293, 0.1293, 0.12928, 0.12922, 0.12909, 0.12879, −0.12844, 0.12804, 0.12799, −0.12792, 0.12787, 0.12786, −0.12775, −0.12768, −0.12761, −0.12761, 0.12737, −0.12698, 0.12693, −0.12682, −0.12681, −0.12663, 0.12652, 0.1263, −0.1259, 0.12556, 0.12549, 0.12543, −0.12541, −0.12527, −0.12525, −0.12521, 0.12509, −0.12508, 0.12489, −0.12486, −0.12479, 0.12466, 0.12464, −0.12461, −0.12451, −0.12441, −0.12419, 0.12397, −0.12394, 0.12365, −0.12355, 0.12353, −0.12353, 0.12327, 0.12319, −0.12318, 0.12289, −0.12279, −0.12278, 0.12277, 0.12275, −0.12271, −0.12258, −0.12257, −0.12256, −0.12255, 0.1225, 0.12228, −0.12225, 0.12223, −0.12183, 0.12172, −0.12154, −0.12142, 0.12141, 0.12141, −0.12129, 0.12112, 0.12107, −0.12087, −0.12081, −0.12075, 0.12072, 0.12068, −0.12067, −0.12055, −0.12035, −0.12034, −0.12016, −0.12015, 0.12002, 0.11969, −0.11968, −0.11961, −0.11932, −0.11929, 0.1191, 0.119, −0.11892, −0.11889, 0.11874, 0.11866, −0.11865, −0.11848, −0.1183, −0.11798, −0.11788, 0.11776, 0.11773, −0.11741, 0.1173, 0.1173, −0.11727, 0.11722, −0.11719, 0.11712, 0.11704, −0.11686, −0.11672, −0.11659, 0.11638, −0.11637, 0.11636, 0.11629, −0.11615, −0.11613, −0.11606, −0.1159, −0.11585, 0.11582, −0.11563, −0.11548, −0.11544, −0.11543, −0.1154, −0.11538, 0.11522, −0.11517, 0.11507, 0.11505, −0.115, −0.11478, −0.11463, 0.11441, 0.11434, 0.11434, 0.11404, 0.114, −0.11391, 0.11359, −0.11351, 0.1135, −0.11348, 0.11346, 0.11337, 0.11336, −0.11335, 0.11313, 0.11308, −0.11271, 0.11269, 0.11261, −0.11254, 0.11248, 0.11242, 0.11231, −0.11228, −0.11217, −0.11217, −0.11214, −0.11212, −0.11195, −0.1119, 0.11185, −0.11174, −0.11152, 0.11147, 0.11144, −0.11132, 0.11115, 0.11099, −0.11099, 0.11098, 0.11091, −0.11091, 0.1108, −0.11068, −0.11066, −0.11032, −0.11031, −0.1102, −0.11007, −0.11001, −0.10992, −0.10991, 0.10989, −0.10984, −0.10976, −0.10974, 0.10963, −0.10959, −0.10948, 0.10948, 0.10947, 0.10944, −0.10938, −0.10936, 0.10917, 0.10907, 0.10897, −0.10869, −0.10866, 0.10865, −0.10864, −0.1086, −0.10835, −0.10833, −0.10818, 0.10816, 0.10813, −0.10807, 0.10804, −0.108, 0.1079, −0.1078, 0.10777, −0.10773, −0.10746, −0.1074, 0.10736, 0.10717, 0.1071, −0.10703, 0.10694, 0.10693, −0.10681, −0.1068, −0.10678, −0.10674, 0.10673, 0.10668, 0.10668, −0.10654, −0.10654, 0.10639, 0.10631, −0.10629, −0.10617, 0.10617, 0.10597, 0.10592, 0.10592, −0.10576, 0.10575, −0.10558, 0.10555, 0.10538, −0.10528, 0.10521, −0.10513, −0.10503, 0.10495, −0.10473, −0.10462, 0.10459, 0.10439, 0.10428, −0.10419, −0.10417, −0.10416, −0.10415, −0.10412, 0.10384, −0.10374, −0.1036, 0.10346, −0.10343, −0.10343, 0.10338, 0.10328, 0.10322, 0.10321, 0.10314, 0.10294, 0.10281, 0.10268, 0.10244, 0.10242, −0.10233, −0.10221, 0.10219, 0.10212, −0.10212, 0.10199, −0.10178, 0.10177, 0.10156, −0.10151, −0.10148, 0.10131, 0.10124, −0.10102, −0.10072, −0.10069, −0.10065, 0.10053, −0.10044, −0.10021, 0.10013, −0.1, −0.09999, 0.09993, −0.09992, 0.0998, −0.09953, −0.09944, 0.09941, 0.09936, −0.09936, −0.09922, −0.09915, 0.09912, −0.09911, −0.09904, 0.09902, −0.09898, 0.09894, 0.09866, −0.09862, 0.09852, −0.09844, −0.0983, 0.09822, 0.09804, 0.09802, 0.09795, −0.09784, −0.09776, −0.09775, −0.09767, −0.09759, −0.09755, 0.09751, 0.09736, 0.09729, −0.09692, −0.0969, −0.09684, 0.09666, −0.09653, −0.09635, −0.09632, 0.0963, 0.09613, −0.09609, −0.09601, −0.09598, 0.09572, 0.0957, 0.09569, 0.09552, −0.09549, −0.09545, 0.09528, −0.09518, −0.09506, 0.09495, 0.09491, −0.0949, 0.09484, 0.09484, −0.09483, 0.09471, −0.09471, 0.09461, −0.09447, −0.09444, −0.09441, −0.09441, 0.0942, 0.09417, 0.0939, −0.09376, −0.09375, 0.09375, 0.09375, 0.09373, 0.09368, −0.09349, −0.09346, −0.09308, −0.09306, 0.09302, 0.09293, −0.09288, −0.09286, −0.09282, 0.09274, −0.09273, 0.0926, −0.09257, −0.09252, −0.09247, 0.09246, −0.09244, 0.09244, 0.09243, −0.09239, −0.09227, −0.09208, −0.09206, −0.092, 0.09196, 0.09189, −0.09186, 0.09172, −0.09166, 0.09163, 0.09157, −0.09154, −0.09146, 0.09128, −0.09115, −0.09114, −0.09106, −0.09105, −0.09105, −0.09098, 0.09083, 0.09076, 0.09076, −0.09066, −0.09056, −0.09038, −0.09033, −0.09029, −0.09019, 0.09009, −0.09004, 0.08999, −0.08991, −0.08985, −0.08983, 0.08981, 0.08979, −0.08969, −0.08969, 0.08967, −0.08965, −0.08962, −0.08957, 0.08947, −0.08943, −0.08942, 0.08933, 0.08916, −0.089, −0.08888, 0.08885, −0.08882, 0.08881, −0.08867, −0.08861, 0.08843, 0.08828, 0.08797, −0.08786, −0.08783, −0.0878, 0.08767, 0.08765, −0.08762, 0.08749, 0.08727, 0.08727, −0.08723, −0.08708, −0.08701, −0.08696, 0.08694, 0.08693, 0.0868, 0.08673, 0.08658, −0.08657, 0.08656, 0.08653, 0.08639, −0.08622, −0.08606, −0.0859, −0.08579, −0.08565, −0.08565, −0.08556, 0.08526, 0.08515, 0.08513, −0.08513, 0.08504, −0.08495, −0.08494, 0.08493, 0.08491, 0.08489, 0.08477, 0.08454, −0.08443, 0.08441, 0.08433, 0.08426, 0.08426, −0.08418, 0.08417, −0.08402, −0.08401, 0.0839, −0.08373, 0.08371, −0.0836, 0.08351, −0.08351, −0.08341, −0.0834, 0.08333, 0.08332, −0.08322, −0.0832, 0.08317, 0.08316, −0.08308, −0.08307, 0.08303, −0.08302, −0.08302, −0.08295, −0.08289, 0.08281, 0.08281, −0.08277, 0.08276, −0.08262, −0.08262, 0.08261, −0.08248, −0.08243, −0.08238, 0.08223, 0.08221, −0.0822, −0.08214, −0.08207, 0.08199, 0.08198, 0.08188, −0.08181, −0.08179, −0.08175, 0.08165, −0.08164, −0.0816, −0.08155, 0.08155, −0.08154, 0.08151, 0.08137, 0.08132, 0.0813, 0.08122, 0.08118, 0.0811, 0.08106, −0.08105, −0.08099, −0.08083, 0.08067, −0.08066, 0.08062, −0.08055, −0.08043, −0.08033, −0.08024, 0.08023, −0.08022, −0.08008, −0.08006, 0.07999, 0.07993, 0.07993, −0.07991, −0.0799, 0.07983, −0.07978, −0.07975, 0.07975, −0.07974, −0.07964, −0.07963, 0.07957, 0.07953, −0.07936, −0.07934, −0.07928, −0.07914, 0.07905, −0.079, −0.07885, −0.07875, 0.07875, 0.07875, −0.0787, −0.07863, −0.07862, 0.07859, −0.0785, −0.07847, 0.07837, −0.07834, 0.07834, −0.07832, −0.07821, −0.07817, −0.07815, −0.07808, 0.07802, 0.07798, 0.07795, −0.07789, −0.07786, 0.07774, 0.07763, −0.0776, 0.07751, −0.07747, 0.07745, −0.07737, −0.07735, 0.07728, −0.07724, 0.07707, 0.077, 0.07694, −0.07674, 0.0767, −0.07646, 0.07646, 0.07635, −0.07632, 0.07627, −0.07624, 0.07624, 0.07619, −0.07614, 0.07601, −0.07594, −0.0759, −0.07587, −0.07586, 0.07579, 0.07578, 0.07569, 0.07568, −0.07564, −0.07561, 0.07559, 0.07556, −0.07548, 0.07546, 0.0754, −0.07533, −0.07527, 0.07524, −0.07515, −0.0751, 0.07508, 0.07502, −0.07493, 0.07485, −0.07475, 0.07472, −0.07465, 0.07463, −0.07447, 0.07431, 0.07417, −0.07413, −0.0741, 0.07409, 0.07407, −0.07406, 0.074, 0.07398, 0.07389, −0.07381, 0.07369, −0.07362, −0.0736, 0.07356, −0.0735, 0.0735, −0.0735, −0.07343, −0.07342, 0.0734, −0.07329, −0.07323, −0.07312, −0.07303, −0.07297, −0.07287, 0.07286, 0.07285, −0.07284, −0.07274, −0.07267, 0.07261, −0.07258, 0.07252, −0.0725, 0.07235, −0.07234, −0.07227, 0.07218, 0.07215, 0.07215, −0.07214, 0.07213, −0.07185, −0.07185, −0.07179, 0.07175, −0.07173, −0.07169, −0.07167, −0.07166, 0.07154, −0.07152, 0.07145, 0.07141, 0.07134, 0.07134, −0.07129, −0.07129, −0.07128, 0.07124, −0.07122, −0.07117, 0.07111, −0.07093, −0.07087, −0.07086, −0.07083, −0.07082, −0.07079, 0.07073, −0.07073, −0.07063, −0.07063, 0.07059, −0.07055, 0.07048, 0.07048, −0.07035, −0.07034, −0.07033, 0.07028, −0.07028, −0.07025, 0.07015, −0.07002, 0.07002, 0.06991, −0.06988, −0.06985, −0.06984, 0.06974, 0.06971, 0.06964, 0.06961, 0.06959, 0.06959, −0.06936, −0.06932, −0.0693, −0.0692, −0.06915, −0.06912, 0.06909, −0.06907, 0.06907, 0.06901, −0.06895, −0.06895, 0.06894, 0.06883, −0.06883, 0.06881, −0.06871, −0.06864, 0.06863, −0.06862, 0.06862, 0.06857, −0.06846, 0.06844, −0.06825, −0.06816, −0.06815, −0.06812, −0.0681, −0.06801, 0.06801, 0.06799, 0.06792, −0.0679, 0.06788, −0.06788, −0.06786, −0.06785, 0.06784, 0.06764, 0.06753, 0.06748, −0.06738, −0.06735, −0.06732, 0.06732, −0.06728, 0.06724, −0.06721, 0.0671, 0.06708, 0.06704, 0.06704, −0.06701, 0.06691, −0.06688, 0.06678, 0.0667, 0.06669, 0.06662, 0.06657, 0.06657, 0.06654, −0.06649, −0.06648, −0.06644, 0.06641, −0.06636, −0.06634, −0.06631, −0.06629, 0.06622, −0.06616, 0.06616, 0.06614, −0.06613, −0.06611, −0.06609, 0.06604, 0.06603, −0.06602, 0.06599, −0.06596, −0.06595, −0.06586, 0.06581, −0.06579, 0.06576, −0.06575, −0.06572, −0.06572, −0.06569, −0.06564, −0.06556, 0.06552, −0.06552, −0.06552, −0.0655, −0.06537, −0.06534, 0.06533, −0.06524, −0.06523, 0.06516, 0.06513, 0.06513, 0.06499, 0.06498, −0.06493, −0.06492, 0.06488, 0.06486, −0.06483, 0.06482, 0.06473, −0.06472, −0.0647, 0.06469, 0.06467, −0.06467, −0.06464, −0.06455, −0.06448, 0.06446, −0.06442, 0.06433, 0.0643, 0.0643, −0.06423, −0.06421, 0.06417, 0.06413, −0.06401, 0.06399, −0.06396, 0.06392, −0.0639, −0.06386, 0.06384, −0.06378, 0.06376, −0.06365, −0.06359, 0.06351, 0.06347, −0.06342, 0.0633, 0.06329, −0.06327, −0.06322, 0.06322, 0.06317, −0.06307, −0.0628, 0.06276, −0.06276, −0.06273, 0.06269, −0.06265, 0.06255, 0.06249, 0.06246, −0.06244, −0.06239, −0.06237, −0.06232, −0.06219, −0.06218, −0.06208, −0.06203, −0.06201, 0.06184, 0.06172, −0.06172, −0.0617, 0.06169, 0.06169, 0.06158, −0.06156, 0.06149, −0.06147, 0.06141, 0.06136, 0.06135, 0.0613, 0.06129, 0.06129, 0.06129, −0.06127, −0.06123, 0.06114, 0.06112, −0.06111, 0.06111, 0.06109, 0.06106, −0.06104, −0.06103, −0.061, 0.06099, 0.06098, −0.06092, −0.06091, 0.06083, 0.0608, 0.06075, 0.06074, 0.06069, −0.06069, 0.06067, 0.06057, −0.06057, 0.06054, 0.06053, −0.06049, −0.06032, 0.06028, −0.06023, −0.06019, −0.06018, −0.06016, −0.06015, 0.06004, −0.06002, 0.06002, −0.05998, −0.05998, −0.05996, −0.05991, 0.05989, −0.05989, −0.05985, −0.05985, −0.05985, −0.05983, −0.05969, −0.05968, 0.05966, 0.0596, −0.05952, −0.05948, −0.05939, 0.05936, 0.05935, 0.05928, 0.05924, −0.05911, −0.05907, −0.05904, 0.05901, 0.05895, 0.05892, −0.0587, −0.05866, −0.05862, −0.05859, 0.05843, 0.05842, −0.05836, −0.05835, −0.05834, −0.05832, −0.05829, 0.05825, −0.05822, 0.05822, 0.05821, 0.05819, −0.05818, 0.05817, −0.05813, 0.0581, −0.05808, −0.05798, 0.05792, −0.05784, 0.0578, −0.05779, −0.05776, 0.05773, 0.05772, −0.05765, −0.05762, −0.05761, −0.05759, −0.05751, 0.05748, 0.05747, −0.05742, −0.05736, −0.05734, 0.05732, −0.05731, −0.05731, −0.0573, −0.05724, −0.05721, −0.05721, −0.0572, −0.05717, 0.05707, −0.05702, 0.05698, −0.05695, 0.05693, −0.05674, −0.05665, −0.05658, −0.05655, −0.05654, 0.05645, −0.05644, −0.05643, 0.05641, −0.05636, −0.05628, −0.05627, 0.0562, −0.05617, 0.0561, −0.05609, −0.05609, 0.05608, −0.05605, 0.05603, −0.05603, 0.05599, 0.05597, 0.0559, 0.05588, −0.05586, −0.05584, −0.05581, −0.05574, 0.05573, −0.05566, −0.05564, −0.05559, 0.05555, 0.05554, −0.05553, 0.05543, 0.05542, 0.05535, 0.05533, 0.05531, −0.05527, 0.05521, −0.05515, −0.05509, −0.05485, −0.05479, −0.0547, 0.05469, 0.0546, 0.05459, −0.05458, 0.05458, 0.05452, 0.05449, −0.05446, 0.05439, −0.0543, −0.05428, 0.05427, −0.05426, −0.05424, −0.05422, 0.05421, −0.05415, −0.05414, 0.05413, 0.0541, −0.05409, 0.05399, 0.05385, −0.05383, 0.05381, −0.05378, −0.05374, −0.05372, 0.05371, 0.05363, −0.05361, −0.05354, 0.05348, −0.05345, −0.05344, 0.05344, 0.05343, −0.05338, 0.05331, −0.05331, −0.05326, −0.05322, −0.05322, −0.0531, 0.05309, 0.05307, −0.05296, −0.05296, 0.05293, 0.0529, −0.0529, 0.0529, 0.05283, −0.05283, 0.05274, 0.05268, −0.05264, −0.05259, −0.05258, 0.05257, −0.05255, −0.05253, −0.05253, −0.05249, 0.05234, 0.0523, −0.05214, 0.05214, −0.05212, 0.05208, 0.05201, −0.05201, 0.05198, 0.05193, −0.05191, −0.05189, 0.05186, 0.05185, 0.0518, −0.0518, 0.05176, 0.05176, 0.05172, −0.05169, −0.05167, −0.05167, −0.05156, 0.05153, 0.05149, 0.05147, 0.05143, −0.05143, 0.05139, 0.05137, −0.05121, −0.05119, 0.05117, 0.05116, −0.05116, −0.05116, 0.05108, 0.05107, 0.05106, −0.05099, −0.05096, 0.05093, −0.0509, 0.05087, −0.05087, −0.05085, 0.05078, 0.05076, −0.05075, −0.05074, −0.0507, −0.05067, −0.05065, 0.05057, 0.05054, 0.05051, −0.05049, −0.05049, −0.05048, −0.05038, −0.05037, 0.05034, 0.05032, −0.05023, 0.05021, −0.05019, 0.05013, 0.05012, −0.05011, −0.05007, 0.05005, 0.05002, 0.04987, 0.04987, −0.04985, −0.04985, −0.04983, 0.04981, 0.04979, 0.04979, −0.04976, 0.04974, 0.04969, 0.04967, −0.04966, −0.04961, 0.0496, −0.04957, 0.04957, 0.0495, −0.0495, −0.04943, 0.04943, 0.04937, 0.04936, 0.04934, −0.04932, 0.04932, −0.04931, 0.04927, 0.04922, −0.04922, 0.04921, −0.04919, −0.04918, −0.04918, −0.04912, −0.0491, −0.04907, 0.04903, 0.04903, 0.049, −0.04893, 0.04888, 0.04888, 0.04881, 0.0488, −0.0488, 0.04879, −0.04879, 0.04875, −0.04868, −0.04862, 0.04857, −0.0485, 0.04847, −0.04846, 0.04846, −0.04842, 0.04839, 0.04833, −0.04831, 0.04829, 0.04828, 0.04825, −0.04822, −0.04822, 0.04816, −0.04815, −0.04815, −0.04814, −0.04813, −0.04811, −0.04808, −0.04803, 0.04797, 0.04796, −0.04792, 0.0479, 0.04787, 0.04787, 0.04786, −0.04772, −0.04769, 0.04758, −0.04758, −0.04754, 0.04753, −0.04746, −0.04733, −0.04732, 0.0473, 0.04727, −0.04726, −0.047, −0.04694, 0.04691, 0.0469, 0.04688, −0.04683, −0.04682, 0.04675, −0.04671, −0.04663, −0.04661, 0.0466, −0.04656, 0.04656, −0.04655, 0.04652, 0.04647, −0.04646, 0.04646, 0.04642, 0.0464, −0.04613, 0.04609, −0.04608, −0.04606, 0.04606, 0.04603, 0.046, −0.046, 0.04597, 0.04597, −0.04594, −0.04593, −0.04593, −0.04591, −0.04591, −0.04588, −0.04588, −0.04578, −0.04576, 0.04575, −0.04575, −0.04572, 0.04566, −0.04564, −0.04561, 0.04553, −0.04547, 0.04546, 0.04546, 0.04546, −0.04545, 0.04541, −0.04528, 0.04509, 0.04507, −0.04506, −0.04505, −0.04502, 0.04502, 0.04501, 0.04498, 0.04498, −0.04496, 0.04496, 0.04495, 0.04489, 0.04479, −0.04477, −0.04475, −0.04473, 0.04472, 0.04468, 0.04466, −0.04466, −0.04464, −0.0446, 0.04459, 0.04459, 0.04441, 0.04441, 0.04439, −0.04438, −0.04437, −0.04435, −0.04434, 0.04433, 0.04431, 0.04431, 0.0443, 0.04427, −0.04423, 0.04417, 0.04417, 0.04415, −0.04415, −0.04414, −0.04413, −0.04411, −0.04407, −0.04407, 0.04407, −0.044, 0.04397, 0.04395, −0.04394, 0.04393, 0.04392, −0.0439, 0.0439, 0.04387, 0.04385, 0.04382, 0.04377, 0.04371, −0.04371, 0.04369, −0.04369, −0.04362, 0.04353, 0.0435, −0.04339, 0.04339, 0.04336, −0.04332, −0.0433, −0.04328, −0.04321, 0.04321, −0.0432, 0.04317, −0.04316, 0.04312, −0.04309, 0.04303, 0.043, 0.04298, −0.04296, 0.04295, 0.04294, 0.04292, −0.04292, −0.04287, −0.04284, 0.04281, −0.04276, 0.0427, −0.04262, −0.04262, 0.04257, −0.04254, −0.04254, 0.04253, −0.04246, 0.04243, 0.04243, 0.0424, −0.04239, −0.04237, −0.04237, 0.04236, −0.04235, −0.04235, −0.0423, 0.04225, −0.04223, −0.04216, 0.04215, −0.04212, −0.0421, 0.04209, 0.04203, −0.04201, 0.042, −0.042, −0.04196, −0.04193, 0.04193, 0.04191, −0.04186, −0.04186, −0.04183, 0.04182, −0.04181, 0.04178, −0.04172, −0.04169, 0.04169, −0.04163, −0.04161, 0.04147, −0.04147, −0.04142, 0.04138, 0.04132, 0.0413, −0.04126, 0.04126, −0.04125, −0.0412, −0.04119, −0.04116, 0.04114, 0.04113, −0.0411, 0.04106, 0.04105, −0.04104, 0.04101, −0.04101, −0.04098, −0.04096, −0.04087, −0.04087, 0.04086, −0.04086, 0.0408, −0.04079, 0.04078, −0.04076, 0.04076, 0.04069, 0.04068, −0.04066, −0.04065, 0.04062, 0.0406, 0.04058, 0.04056, 0.04055, 0.04054, −0.04051, 0.0405, −0.04048, −0.04048, −0.04045, 0.04044, −0.04035, 0.0403, −0.04027, 0.04027, −0.04023, −0.04023, 0.04022, 0.04022, −0.04019, −0.04017, 0.04014, 0.04012, −0.04003, −0.03998, 0.03991, 0.03983, 0.03981, 0.03977, −0.03976, 0.03976, −0.03974, 0.03973, −0.03971, 0.03967, 0.03965, −0.03962, −0.0396, −0.03958, −0.03957, 0.03956, −0.03956, −0.03952, −0.03952, −0.03951, −0.0395, −0.0395, 0.03947, −0.03941, 0.03939, −0.03931, −0.03929, 0.03928, 0.03926, −0.03925, −0.03922, −0.03921, 0.03919, 0.03918, −0.03917, 0.03906, 0.03906, −0.03889, −0.03887, −0.03886, −0.03886, −0.03885, −0.03885, 0.0388, 0.03877, −0.03876, 0.03876, −0.03875, −0.03871, −0.03868, 0.03866, −0.03859, 0.03859, 0.03858, −0.03851, −0.03848, −0.03843, −0.03841, 0.03841, 0.03841, 0.03833, −0.03832, −0.03832, −0.03831, −0.03826, 0.03821, −0.0382, 0.03816, 0.0381, 0.0381, −0.03808, 0.03808, −0.03806, 0.03805, −0.03804, −0.03803, −0.03803, −0.03802, −0.03796, −0.03781, 0.03774, −0.03773, 0.03765, 0.03763, −0.03763, −0.03763, −0.03752, −0.0375, 0.0375, 0.03749, −0.03749, 0.03746, 0.03741, −0.03739, −0.03739, −0.03737, −0.03733, −0.03733, 0.03722, −0.03719, −0.03716, −0.03715, 0.03711, 0.03698, −0.03697, 0.03697, −0.03693, 0.03692, 0.03691, −0.03688, 0.03687, 0.03685, −0.0368, −0.03674, 0.03673, 0.03672, 0.03669, 0.03664, 0.03664, 0.03663, −0.03655, 0.03651, 0.03645, 0.03644, −0.03641, 0.03638, 0.03633, 0.03632, −0.03626, −0.03622, −0.03618, −0.03617, 0.03615, −0.03615, 0.03606, 0.03604, 0.03599, 0.03598, −0.03593, −0.0359, 0.0359, 0.03588, 0.03587, −0.03587, −0.03581, −0.0358, 0.0358, −0.03572, 0.0357, −0.03569, −0.03568, 0.03564, −0.03563, −0.03561, −0.03561, 0.03561, −0.0356, −0.0356, −0.03555, −0.03554, 0.0355, 0.03547, −0.03546, 0.03541, −0.03533, 0.03531, 0.03531, 0.03529, 0.03528, −0.03528, −0.03528, 0.03528, −0.03522, −0.03519, −0.03518, −0.03518, −0.03515, 0.03506, 0.03504, 0.03502, −0.03494, 0.03489, −0.03488, 0.03484, 0.03481, 0.0348, −0.0348, −0.03479, 0.03476, −0.03475, −0.03473, −0.03471, −0.03469, 0.03467, 0.0346, 0.03456, −0.0345, −0.03447, 0.03446, −0.03442, 0.03441, −0.0344, −0.03438, 0.03433, 0.03431, −0.03428, −0.03427, −0.03425, −0.03422, −0.03421, 0.03421, 0.03415, 0.03414, −0.03413, 0.03411, −0.03411, 0.03408, 0.03407, 0.03407, −0.03406, −0.03405, 0.03402, −0.034, 0.03396, 0.03395, 0.03395, −0.03394, −0.03393, −0.03393, −0.03392, 0.03391, −0.03389, 0.03389, −0.03386, 0.03382, −0.03382, −0.03381, 0.0338, 0.0338, 0.03379, −0.03372, −0.03371, −0.0337, 0.03363, −0.03363, −0.03362, −0.03362, 0.0336, −0.0336, 0.0336, 0.03359, 0.03359, −0.03356, −0.03354, 0.03353, −0.03351, 0.03348, −0.03347, 0.03346, 0.03346, −0.03344, 0.03343, −0.03342, 0.03335, 0.03327, 0.0332, 0.03316, −0.03316, −0.03315, 0.03315, −0.03313, −0.03313, 0.03305, 0.03302, 0.03299, −0.03296, −0.03293, 0.03291, −0.03291, 0.0329, −0.03289, 0.03289, −0.03289, 0.03288, 0.03286, 0.03284, 0.03283, 0.03278, 0.03275, 0.03273, 0.0327, 0.03266, −0.03264, −0.03262, 0.03262, 0.03261, −0.03261, 0.03257, 0.03254, −0.03253, −0.03252, 0.0324, −0.0324, 0.03237, −0.03229, −0.03226, 0.03225, −0.03224, 0.03224, −0.03221, −0.03219, −0.03217, −0.03212, 0.03211, −0.0321, −0.03206, −0.03204, 0.03202, 0.03201, 0.032, 0.032, 0.03189, −0.03188, 0.03184, 0.03184, 0.03183, −0.03181, 0.0318, 0.03178, −0.03174, −0.03173, 0.0317, −0.0317, 0.0317, 0.03167, 0.03166, 0.03166, −0.03163, −0.03159, −0.03159, 0.03158, −0.03157, 0.03153, −0.03149, −0.03144, −0.0313, −0.03126, 0.03125, −0.03123, −0.0312, 0.03114, −0.03113, 0.0311, −0.03107, −0.03105, 0.03099, −0.03098, 0.03096, −0.03086, 0.03085, 0.03081, 0.03078, −0.03076, 0.03075, 0.03075, 0.03072, 0.03067, −0.03059, −0.03059, −0.03058, 0.0305, 0.03048, −0.03048, 0.03045, −0.03045, −0.03044, −0.0304, 0.03037, −0.03031, −0.03027, −0.03027, 0.03022, 0.03019, −0.03012, 0.03011, 0.03004, 0.03, 0.02998, 0.02996, −0.02996, 0.02994, −0.02994, −0.02992, 0.0299, 0.0299, 0.02984, 0.02982, 0.0298, −0.02979, −0.02974, 0.02972, −0.02969, 0.02969, −0.02968, −0.02968, −0.02968, −0.02967, 0.02964, 0.02964, −0.02959, 0.02955, −0.02955, 0.02954, 0.02947, −0.02945, −0.02939, −0.02937, 0.02937, 0.02934, 0.02933, 0.0293, 0.02926, −0.02926, −0.02923, −0.02921, 0.0292, −0.02918, −0.02917, 0.02915, −0.02914, −0.02914, 0.02913, −0.02912, 0.02911, −0.0291, 0.02909, 0.02903, −0.02902, 0.02902, −0.02899, 0.02895, 0.02888, 0.02888, −0.02887, −0.02885, 0.02882, 0.0288, −0.02876, 0.02875, −0.02866, 0.02863, −0.02861, −0.02861, −0.02861, −0.02859, −0.02856, −0.0285, −0.02846, 0.02844, −0.02843, 0.0284, −0.02839, 0.02836, −0.02832, 0.02832, −0.0283, 0.02829, 0.02828, 0.02826, −0.02824, 0.02822, −0.0282, 0.02816, 0.02815, 0.02815, −0.02809, 0.02808, 0.02808, 0.02807, −0.02804, 0.028, 0.02795, 0.02794, −0.02793, −0.02788, −0.02785, −0.02785, 0.02784, −0.02784, −0.02782, 0.02781, −0.0278, 0.02779, 0.02778, 0.02776, 0.02774, 0.02773, −0.02771, −0.02771, −0.02766, 0.02765, 0.02764, 0.02758, 0.02757, −0.02753, −0.02752, −0.02747, 0.02746, 0.02742, −0.02736, −0.02735, −0.02735, −0.02734, −0.02731, −0.02727, 0.02721, 0.02721, 0.0272, −0.02719, 0.02719, −0.02714, 0.02709, −0.02708, −0.02705, 0.027, 0.02699, −0.02698, 0.02695, −0.02695, −0.02689, −0.02688, −0.02688, −0.02685, −0.02683, −0.0268, 0.02678, −0.02677, −0.02677, 0.02675, −0.02675, −0.02672, 0.02669, −0.02669, −0.02668, 0.02668, 0.02666, −0.02666, −0.02664, −0.02663, 0.02661, −0.02659, 0.02657, 0.02657, 0.02656, −0.0265, −0.02642, −0.02641, −0.02631, 0.0263, 0.02629, −0.02625, 0.02625, −0.02624, −0.02624, −0.02622, 0.0262, 0.02617, −0.02614, 0.02613, 0.02611, 0.02606, −0.02606, 0.02596, 0.02594, −0.02592, 0.02592, 0.02591, 0.0259, −0.02586, 0.02581, 0.02581, 0.02578, 0.02574, 0.02574, 0.02572, 0.02569, −0.02565, 0.02564, 0.02563, 0.02563, 0.02561, 0.02558, −0.02555, 0.02553, −0.02548, −0.02547, −0.02547, −0.02541, −0.02529, 0.02521, −0.02521, −0.0252, 0.0252, 0.02515, 0.02513, −0.02513, 0.02512, 0.02512, 0.02511, −0.02508, 0.02507, 0.02506, −0.02506, −0.02505, −0.02504, −0.02503, −0.02501, −0.02496, −0.02494, −0.02491, 0.02488, 0.02488, −0.02486, −0.02483, −0.02483, −0.02481, −0.02481, −0.02479, −0.02478, 0.02478, −0.02476, −0.02476, 0.02475, −0.02473, −0.02469, 0.02461, 0.02461, −0.02461, 0.02461, 0.02457, 0.02456, 0.02455, 0.02454, −0.02452, 0.02446, −0.02444, −0.02443, 0.02442, −0.0244, 0.02439, −0.02436, 0.02435, −0.02433, 0.02433, −0.02427, 0.02425, −0.02421, −0.02419, −0.02416, −0.0241, 0.02409, −0.02403, 0.02401, 0.02399, 0.02398, −0.02397, −0.02393, 0.0239, −0.02389, −0.0238, 0.02379, 0.02375, −0.02375, 0.02373, 0.02367, −0.02363, −0.02363, −0.0236, −0.0236, 0.02359, 0.02353, 0.02352, −0.02351, −0.02349, 0.02347, −0.02345, 0.02344, −0.02343, 0.02341, 0.02339, −0.02339, −0.02338, 0.02338, 0.02337, 0.02336, −0.02332, −0.02331, −0.02327, 0.02326, −0.02324, −0.02317, 0.02306, 0.02305, −0.02305, −0.02305, −0.02302, 0.02302, 0.02297, 0.02297, 0.02296, 0.02296, −0.0229, 0.02286, −0.02286, 0.02286, 0.02285, 0.02283, 0.02282, 0.0228, −0.02279, 0.02278, 0.02277, −0.02276, −0.02274, −0.02273, 0.02271, 0.0227, −0.02269, −0.02266, −0.02266, −0.02264, −0.02264, −0.02259, −0.02256, 0.02256, 0.02254, 0.02252, −0.02252, −0.0225, −0.02248, 0.02246, −0.02245, −0.02243, −0.02241, 0.0224, −0.02238, 0.02237, −0.02236, −0.02236, 0.02232, 0.02231, −0.02228, 0.02226, 0.02225, −0.02221, 0.0222, −0.02217, 0.02215, −0.0221, 0.0221, 0.0221, 0.02209, −0.02208, −0.02202, −0.02198, −0.02197, −0.02195, 0.02194, 0.02192, 0.0219, −0.0219, 0.02186, −0.02186, −0.02185, −0.02184, 0.02183, −0.02183, −0.02181, −0.02179, −0.02179, −0.02176, −0.02175, −0.02174, 0.02174, 0.02172, 0.02171, −0.02169, 0.02169, −0.02167, −0.02167, −0.02166, −0.02166, −0.02164, −0.02163, 0.02162, −0.02162, −0.02161, 0.02157, −0.02156, −0.02155, 0.02154, 0.0215, 0.02149, 0.02149, −0.02146, 0.02145, 0.02144, −0.02139, −0.02137, 0.02135, 0.02134, −0.02129, 0.02112, −0.02109, −0.02109, 0.02108, −0.02104, 0.02102, 0.02095, −0.02093, −0.02087, 0.02087, −0.02082, −0.02081, −0.0208, −0.02078, −0.02078, 0.02076, 0.02071, 0.0207, 0.02068, 0.02067, −0.02062, 0.02061, −0.0206, −0.02056, −0.02056, −0.02053, 0.02051, −0.02049, 0.02042, −0.02041, 0.02041, 0.0204, −0.02037, 0.02035, −0.02035, 0.02035, 0.02034, 0.02032, 0.0203, −0.0203, 0.02028, 0.02028, 0.02026, −0.02026, 0.02024, 0.02024, 0.02019, 0.02011, −0.02011, 0.02011, −0.02011, 0.0201, 0.0201, −0.02008, 0.02006, −0.02005, −0.02004, −0.02003, 0.02002, 0.02002, −0.02001, 0.01999, 0.01998, 0.01996, 0.01996, −0.01993, 0.01992, 0.01991, −0.01991, −0.01984, −0.01981, −0.0198, 0.0198, 0.01979, 0.01977, 0.01974, 0.01968, −0.01964, 0.01964, 0.01963, −0.01963, 0.01962, −0.0196, −0.0196, −0.0196, −0.01958, 0.01957, 0.01954, 0.01945, −0.01944, 0.01944, 0.01938, 0.01936, −0.01935, 0.01933, 0.01931, 0.01929, 0.01924, 0.01924, −0.01916, 0.01914, 0.01911, −0.0191, 0.01909, 0.01906, 0.01901, 0.01901, 0.019, −0.01899, −0.01896, −0.01892, 0.01891, 0.01888, −0.01886, 0.01885, 0.01881, 0.01879, 0.01877, −0.01874, −0.01873, 0.01873, 0.01868, 0.01868, 0.01867, 0.01863, 0.01862, 0.0186, 0.01859, −0.01857, −0.01852, −0.01851, 0.01849, 0.01846, −0.01846, −0.0184, 0.01837, −0.01837, 0.01836, −0.01836, 0.01832, 0.0183, −0.01828, −0.01828, −0.01827, −0.01825, −0.01824, −0.01822, 0.01812, 0.01809, −0.01809, −0.01807, −0.01805, −0.01805, 0.01805, −0.01804, −0.01804, 0.01804, −0.01803, −0.01801, 0.01795, −0.01793, 0.01793, −0.01792, −0.01791, 0.01789, 0.01784, −0.01784, 0.01777, 0.01773, 0.01773, 0.0177, 0.01769, 0.01766, 0.01763, −0.01761, −0.0176, −0.01758, −0.01756, −0.01754, 0.01753, −0.01748, −0.01748, 0.01748, −0.01744, −0.01743, −0.01742, −0.01741, −0.01738, 0.01738, 0.01737, −0.01735, 0.01735, −0.01729, −0.01728, −0.01723, 0.01722, 0.01722, 0.01721, 0.01719, −0.01719, −0.01718, −0.01717, 0.01717, 0.01715, 0.01714, 0.01714, 0.01713, 0.01711, −0.01711, −0.0171, −0.0171, −0.01708, −0.01706, 0.01706, −0.01701, 0.017, −0.017, −0.01696, 0.01696, 0.01694, −0.01693, 0.01689, −0.01687, 0.01684, −0.01682, −0.01679, 0.0167, 0.01669, −0.01668, −0.01667, −0.01666, 0.01664, −0.01661, −0.01658, 0.01656, −0.01656, 0.01651, 0.0165, −0.01649, −0.01649, 0.01649, −0.01648, −0.01648, 0.01646, −0.01645, 0.01641, 0.01638, −0.01638, −0.01638, −0.01635, −0.01631, 0.01629, 0.01626, −0.01626, −0.01625, 0.01625, −0.01624, −0.01624, 0.01624, 0.01619, 0.01616, 0.01611, 0.0161, −0.0161, −0.01608, −0.01605, 0.01603, 0.01602, 0.01601, −0.016, −0.01597, −0.01596, −0.01595, −0.01587, −0.01586, 0.01585, −0.01585, −0.01584, −0.01581, 0.01581, −0.01579, −0.01578, 0.01571, −0.0157, 0.0157, −0.01569, 0.01562, 0.01562, −0.01562, 0.01561, 0.01557, 0.01552, −0.01551, −0.01548, 0.01547, −0.01544, 0.01542, −0.01542, 0.01542, −0.01539, −0.01536, −0.01536, −0.01534, −0.01534, 0.01533, 0.01531, 0.01529, −0.01529, −0.01527, 0.01525, −0.0152, 0.0152, −0.01519, 0.01516, 0.01514, 0.01512, −0.01509, −0.01508, −0.01501, 0.015, −0.01499, 0.01499, −0.01498, −0.01498, −0.01497, −0.01496, −0.01496, −0.01494, −0.01494, −0.01493, 0.01492, 0.0149, −0.01486, 0.01483, −0.01482, 0.01478, 0.01476, 0.01475, 0.01475, 0.01474, −0.01473, 0.01472, −0.01471, 0.01462, 0.01461, −0.01461, 0.01456, −0.01456, −0.01451, −0.01451, 0.0145, −0.01447, 0.01446, −0.01443, −0.01442, 0.0144, 0.01438, 0.01434, −0.01434, 0.01432, −0.0143, −0.01421, −0.01418, 0.01418, −0.01418, 0.01418, −0.01417, 0.01413, 0.01412, 0.01411, −0.0141, 0.0141, 0.01407, 0.01406, 0.01403, 0.01397, −0.01395, 0.01394, −0.01393, −0.01389, −0.01386, 0.01384, −0.01384, 0.01382, −0.0138, −0.0138, −0.01379, 0.01379, −0.01379, −0.01378, −0.01376, −0.01375, 0.01374, −0.01367, 0.01367, 0.01364, −0.01362, 0.01358, −0.01357, −0.01357, −0.01355, −0.01355, 0.01353, −0.01351, 0.01351, −0.01347, 0.01344, 0.01342, −0.01342, −0.01341, 0.01341, −0.01339, −0.01334, 0.01333, −0.01328, −0.01327, −0.01327, 0.01325, 0.01323, −0.01322, −0.01317, −0.01317, −0.01313, 0.01312, −0.0131, 0.01308, −0.01306, −0.01306, −0.01302, 0.013, 0.01299, −0.01299, 0.01298, −0.01297, 0.01296, −0.01294, −0.01293, −0.0129, −0.01285, 0.01282, −0.01281, −0.0128, 0.01275, −0.01273, −0.01272, 0.0127, −0.0127, 0.01268, −0.01268, 0.01267, −0.01266, 0.01264, 0.01264, −0.01263, 0.01261, 0.01259, 0.01259, −0.01258, 0.01258, 0.01254, −0.01253, 0.01248, −0.01248, 0.01248, −0.01247, −0.01246, −0.01243, −0.01242, −0.01241, −0.01239, 0.01238, 0.01238, 0.01237, −0.01235, 0.01235, −0.01235, 0.01231, 0.0123, 0.01229, −0.01229, −0.01225, −0.01225, 0.01224, −0.01224, 0.01224, 0.01223, 0.01222, 0.01218, −0.01217, 0.01217, −0.01214, 0.01213, −0.01213, 0.01212, 0.01212, −0.01206, 0.01206, −0.01206, 0.01205, −0.01203, 0.01203, 0.01202, 0.01198, 0.01191, −0.01191, 0.0119, 0.0119, 0.01186, −0.01184, 0.01184, 0.0118, 0.0118, −0.01176, 0.01174, −0.01173, −0.01171, 0.01169, −0.01169, 0.01163, 0.01163, 0.01162, −0.0116, 0.01155, 0.01155, 0.01151, 0.0115, 0.01148, 0.01145, −0.01143, −0.01143, 0.0114, −0.01138, 0.01138, −0.01137, 0.01135, −0.01134, −0.01133, −0.01131, −0.01131, −0.01131, −0.01127, 0.01127, 0.01127, 0.01127, 0.01124, 0.01123, −0.01121, 0.0112, −0.01116, −0.01114, −0.01113, −0.01111, 0.01111, −0.01106, −0.01106, 0.01105, −0.01103, −0.01102, −0.01102, −0.011, −0.01098, −0.01097, 0.01096, −0.01092, −0.01091, −0.0109, −0.01088, −0.01087, −0.01086, 0.01083, −0.01073, −0.01072, 0.01069, −0.01068, −0.01064, 0.01064, 0.0106, −0.01059, 0.01058, −0.01055, 0.01055, −0.01055, −0.01054, 0.01051, 0.01051, −0.01045, 0.01045, −0.01044, 0.01043, 0.01041, −0.01039, 0.01038, −0.01036, 0.01036, −0.01035, −0.01033, 0.01031, −0.01029, 0.01029, −0.01028, −0.01026, 0.01024, 0.01023, 0.01022, −0.01021, 0.01021, −0.01019, −0.01018, 0.01016, 0.01014, −0.01014, −0.01013, 0.01011, 0.0101, −0.0101, −0.01002, −0.01001, 0.01, −0.00998, 0.00994, −0.00994, −0.00992, −0.00992, 0.00982, 0.00979, −0.00978, −0.00976, 0.00976, 0.00975, 0.00975, −0.00973, 0.00973, 0.00972, 0.0097, 0.0097, −0.00967, 0.00963, −0.00962, −0.00961, 0.00958, 0.00955, −0.00952, 0.00952, 0.00948, −0.00948, 0.00944, 0.00943, 0.00942, 0.0094, 0.00939, −0.00938, 0.00938, −0.00935, −0.00935, 0.00935, 0.00933, −0.00932, 0.00932, −0.0093, 0.00929, 0.00926, −0.00926, −0.00924, −0.0092, 0.0092, 0.00914, 0.0091, −0.00902, −0.009, −0.00899, −0.00895, 0.00894, 0.00893, −0.00892, 0.00892, −0.00891, −0.00891, 0.0089, 0.00889, −0.00888, 0.00887, 0.00887, −0.00884, −0.00881, 0.0088, −0.0088, −0.0088, −0.00878, −0.00872, −0.00871, −0.0087, 0.0087, 0.00868, 0.00866, −0.00866, 0.00865, 0.00865, 0.00864, −0.00862, −0.00858, 0.00857, −0.00856, 0.00855, 0.00854, 0.00853, −0.00852, −0.00851, 0.00847, −0.00846, −0.00846, 0.00845, 0.00841, −0.00837, −0.00836, 0.00834, 0.00834, 0.00833, −0.00832, −0.00832, 0.00822, 0.0082, 0.0082, 0.0082, −0.00819, 0.00817, −0.00817, −0.00816, 0.00815, −0.00814, 0.00813, −0.00813, −0.00812, −0.008, −0.00798, 0.00796, −0.00795, 0.00794, 0.00794, −0.00789, 0.00787, 0.00785, 0.00779, −0.00778, 0.00775, −0.00774, 0.0077, −0.00769, 0.00769, −0.00768, 0.00767, −0.00766, 0.00762, 0.00761, −0.00754, 0.00753, 0.00753, −0.00751, −0.0075, 0.00749, −0.00744, −0.00741, −0.00739, 0.00735, −0.00732, −0.0073, −0.0073, 0.00729, −0.00728, −0.00725, 0.00725, 0.00723, −0.00721, 0.00718, 0.00716, −0.00714, 0.00712, −0.00711, 0.00708, 0.00708, −0.00703, 0.00703, 0.007, 0.00696, 0.00696, 0.00693, 0.00692, 0.00689, −0.00687, 0.00683, −0.00683, 0.0068, −0.0068, 0.00679, −0.00677, 0.00675, −0.00672, −0.0067, 0.00669, 0.00669, 0.00669, 0.00669, 0.00669, −0.00669, −0.00667, −0.00667, −0.00662, −0.00661, 0.00658, −0.00655, 0.00655, 0.00652, −0.00647, 0.00647, 0.00645, −0.00644, 0.00644, −0.00644, −0.00641, −0.0064, −0.00639, −0.00636, 0.0063, −0.00629, −0.00626, −0.00623, −0.00617, −0.00617, 0.00616, −0.00615, −0.00614, 0.00613, 0.00613, −0.00611, −0.00609, −0.00609, −0.00609, −0.00605, −0.00605, 0.00603, −0.00599, −0.00599, 0.00596, −0.0059, 0.00588, −0.00587, −0.00587, −0.00586, −0.00585, 0.00584, 0.00581, −0.00579, −0.00577, −0.00573, 0.00573, −0.00573, 0.00571, −0.0057, 0.0057, 0.0057, −0.00568, −0.00567, −0.00562, −0.00553, 0.00553, −0.00553, 0.00552, 0.00549, −0.00547, 0.00545, 0.00544, −0.00543, −0.00541, −0.00541, −0.0054, −0.00539, −0.00539, −0.00538, 0.00535, −0.00535, −0.00534, −0.00532, 0.0053, −0.00529, −0.00526, −0.00526, 0.00525, −0.00523, −0.00522, −0.00521, −0.00521, −0.0052, −0.00519, −0.00519, −0.00517, 0.00514, −0.00512, −0.00511, −0.0051, −0.0051, −0.00507, −0.00503, 0.00499, 0.00498, 0.00498, 0.00495, −0.00493, −0.00491, −0.0049, −0.00489, 0.00487, 0.00485, 0.00485, 0.00484, 0.00482, −0.00482, −0.00481, −0.00479, −0.00476, −0.00474, 0.00471, 0.00469, −0.00466, 0.00465, 0.00464, 0.00463, −0.00461, −0.0046, −0.00459, −0.00458, 0.00457, −0.00457, 0.00456, 0.00446, −0.00445, 0.00443, −0.00443, 0.00443, −0.00442, 0.00439, −0.00432, −0.00432, −0.0043, −0.00427, 0.00427, 0.00424, −0.00423, 0.00421, 0.00418, −0.00415, 0.00414, −0.00412, 0.00409, 0.00407, 0.00407, −0.00406, 0.00405, −0.00403, 0.00403, −0.00403, −0.00399, 0.00397, 0.00395, −0.00392, −0.00391, 0.0039, 0.0039, 0.00389, 0.00388, 0.00383, −0.0038, −0.0038, −0.00378, −0.00375, 0.00374, −0.00373, 0.00373, 0.00371, −0.00368, 0.00361, 0.0036, 0.00358, 0.00357, −0.00357, −0.00352, 0.00352, −0.00351, 0.00347, −0.00347, −0.00347, −0.0034, −0.00338, −0.00338, 0.00331, 0.00325, 0.00323, 0.00322, 0.00321, −0.00316, 0.00316, −0.00315, −0.00315, −0.00314, −0.0031, 0.00309, 0.00307, −0.00305, −0.00301, −0.003, −0.00299, 0.00299, 0.00296, −0.00295, −0.00294, 0.00293, −0.00291, −0.00291, 0.00289, −0.00289, −0.00287, 0.00287, 0.00287, −0.00285, −0.00284, −0.00282, −0.00266, −0.00265, 0.00263, −0.00262, 0.00261, −0.0026, −0.0026, −0.00258, 0.00255, −0.00255, −0.00251, −0.0025, 0.00249, −0.00248, 0.00247, 0.00237, 0.00236, 0.00236, −0.00233, 0.00231, −0.0023, −0.00229, −0.00228, 0.00223, 0.00222, 0.0022, 0.00219, 0.00217, 0.00214, 0.0021, −0.00208, −0.00208, −0.00207, 0.00206, −0.00203, −0.00202, −0.00201, −0.002, 0.002, 0.0019, 0.00188, −0.00186, 0.00184, 0.0018, −0.00178, −0.00175, 0.00172, −0.00172, 0.0017, −0.00168, −0.00164, −0.00162, 0.00162, 0.00155, −0.00155, −0.00151, 0.00148, 0.00148, −0.00148, −0.00148, −0.00147, −0.00144, 0.00144, 0.00144, −0.00142, −0.00141, 0.0014, 0.00139, −0.00136, 0.00134, 0.00134, 0.00133, −0.00132, −0.00128, −0.00126, −0.00123, 0.00123, −0.00121, −0.0012, 0.00118, 0.00116, −0.00113, 0.00113, 0.00112, 0.00111, −0.00109, 0.00108, 0.00107, −0.00107, 0.00106, −0.00105, 0.00104, 0.00101, 0.00097, 0.00096, −0.00083, 0.00081, 0.00077, −0.00073, −0.00069, 0.00068, 0.00068, 0.00065, −0.00065, −0.00063, 0.00062, 0.00059, −0.00059, 0.00058, 0.00057, 0.00057, −0.00054, 0.00053, 0.00051, 0.00051, 5.00E-04, −0.00048, 0.00045, −0.00045, 0.00041, 4.00E-04, −0.00039, 0.00038, −0.00038, −0.00038, −0.00036, −0.00036, −0.00033, −0.00031, −0.00029, 0.00025, 0.00019, −0.00019, −0.00018, −0.00014, 0.00011, 5.00E-05, −3.00E-05, −3.00E-05, −3.00E-05, and −1.00E-05.

The predicting the presence, absence or development of CIN3 and/or cancer, particularly cervical or endometrial cancer, most preferably cervical cancer, in an individual may particularly involve a threshold cancer index value being applied in order to assess or stratify an individual has having or not having cancer or of having a high or low risk of CIN3 and/or cancer development.

The assays of the invention may involve a threshold index being applied in order to assess the presence or absence of CIN3 and/or cancer in an individual. The assessment may be characterised by receiver operating characteristics, particularly and area under the curve (AUC), sensitivity, and specificity, indicative of the reliability of the threshold being applied in order to assess the presence or absence of CIN3 and/or cancer in an individual.

In any of the assays described herein, wherein when the cancer index value for the individual is about −0.331 or more, the individual is assessed as having cancer and/or CIN3, or as having a risk of cancer and/or CIN3 development, particularly cervical or endometrial cancer, most preferably cervical cancer, in an individual is based on the WID-CIN-Index. The panel of one or more CpGs used to derive the cancer index value may comprise:
a. at least 500 of the CpGs identified at nucleotide positions 61 to 62 in SEQ ID NOs 1 to 5000, and wherein the sensitivity is at least 87% and the specificity is at least 49%;
b. at least the CpGs defined by SEQ ID NOs 1 to 500 and identified at nucleotide positions 61 to 62, and wherein the sensitivity is at least 86% and specificity is at least 77%;
c. at least the CpGs defined by SEQ ID NOs 1 to 1000 and identified at nucleotide positions 61 to 62, and wherein the sensitivity is at least 87% and specificity is at least 77%;
d. at least the CpGs defined by SEQ ID NOs 1 to 1500 and identified at nucleotide positions 61 to 62, and wherein the sensitivity is at least 89% and specificity is at least 76%.

In any of the assays described herein, wherein when the cancer index value for the individual is about −0.331 or more, the individual is assessed as having cancer and/or CIN3 or as having a high risk of cancer and/or CIN3 development, or wherein when the cancer index value for the individual is less than about −0.331 the individual is assessed as not having cancer and/or CIN3 or as having a low risk of cancer development and/or CIN3, particularly cervical or endometrial cancer, most preferably cervical cancer, in an individual is based on the WID-CIN-Index. The panel of one or more CpGs used to derive the cancer index value may comprise:
1. at least 500 of the CpGs identified at nucleotide positions 61 to 62 in SEQ ID NOs 1 to 5000, and wherein the sensitivity is at least 87% and the specificity is at least 49%;
2. at least 1000 of the CpGs identified at nucleotide positions 61 to 62 in SEQ ID NOs 1 to 5000, and wherein the sensitivity is at least 86% and specificity is at least 49%;
3. at least 1500 of the CpGs identified at nucleotide positions 61 to 62 in SEQ ID NOs 1 to 5000, and wherein the sensitivity is at least 90% and specificity is at least 51%;
4. at least 2000 of the CpGs identified at nucleotide positions 61 to 62 in SEQ ID NOs 1 to 5000, and wherein the sensitivity is at least 89% and specificity is at least 51%;
5. at least 2500 of the CpGs identified at nucleotide positions 61 to 62 in SEQ ID NOs 1 to 5000, and wherein the sensitivity is at least 89% and specificity is at least 53%;
6. at least 3000 of the CpGs identified at nucleotide positions 61 to 62 in SEQ ID NOs 1 to 5000, and wherein the sensitivity is at least 87% and specificity is at least 52%;
7. at least 3500 of the CpGs identified at nucleotide positions 61 to 62 in SEQ ID NOs 1 to 5000, and wherein the sensitivity is at least 87% and specificity is at least 52%;
8. at least 4000 of the CpGs identified at nucleotide positions 61 to 62 in SEQ ID NOs 1 to 5000, and wherein the sensitivity is at least 86% and specificity is at least 57%;
9. at least 4500 of the CpGs identified at nucleotide positions 61 to 62 in SEQ ID NOs 1 to 5000, and wherein the sensitivity is at least 86% and specificity is at least 66%;
10. at least 4900 of the CpGs identified at nucleotide positions 61 to 62 in SEQ ID NOs 1 to 5000, and wherein the sensitivity is at least 90% and specificity is at least 75%; or
11. at least 5000 of the CpGs identified at nucleotide positions 61 to 62 in SEQ ID NOs 1 to 5000, and wherein the sensitivity is at least 90% and specificity is at least 75%.

In any of the assays described herein, wherein when the cancer index value for the individual is about −0.311 or more, the individual is assessed as having cancer and/or CIN3 or as having a high risk of cancer and/or CIN3 development, or wherein when the cancer index value for the individual is less than about −0.311, the individual is assessed as not having cancer and/or CIN3 or as having a low risk of cancer development and/or CIN3, particularly cervical or endometrial cancer, most preferably cervical cancer, in an individual is based on the WID-CIN-Index. The panel of one or more CpGs used to derive the cancer index value may comprise:
1. at least the CpGs defined by SEQ ID NOs 1 to 500 and identified at nucleotide positions 61 to 62, and wherein the sensitivity is at least 86% and specificity is at least 77%;
2. at least the CpGs defined by SEQ ID NOs 501 to 1000 and identified at nucleotide positions 61 to 62, and wherein the sensitivity is at least 84% and specificity is at least 77%;
3. at least the CpGs defined by SEQ ID NOs 1001 to 1500 and identified at nucleotide positions 61 to 62, and wherein the sensitivity is at least 86% and specificity is at least 68%;
4. at least the CpGs defined by SEQ ID NOs 1501 to 2000 and identified at nucleotide positions 61 to 62, and wherein the sensitivity is at least 86% and specificity is at least 60%;
5. at least the CpGs defined by SEQ ID NOs 2001 to 2500 and identified at nucleotide positions 61 to 62, and wherein the sensitivity is at least 90% and specificity is at least 61%;
6. at least the CpGs defined by SEQ ID NOs 2501 to 3000 and identified at nucleotide positions 61 to 62, and wherein the sensitivity is at least 86% and specificity is at least 55%;
7. at least the CpGs defined by SEQ ID NOs 3001 to 3500 and identified at nucleotide positions 61 to 62, and wherein the sensitivity is at least 86% and specificity is at least 52%;
8. at least the CpGs defined by SEQ ID NOs 3501 to 4000 and identified at nucleotide positions 61 to 62, and wherein the sensitivity is at least 86% and specificity is at least 53%;
9. at least the CpGs defined by SEQ ID NOs 4001 to 4500 and identified at nucleotide positions 61 to 62, and wherein the sensitivity is at least 87% and specificity is at least 51%;
10. at least the CpGs defined by SEQ ID NOs 4501 to 5000 and identified at nucleotide positions 61 to 62, and wherein the sensitivity is at least 87% and specificity is at least 49%;
11. at least the CpGs defined by SEQ ID NOs 1 to 100 and identified at nucleotide positions 61 to 62, and wherein the sensitivity is at least 86% and specificity is at least 77%;
12. at least the CpGs defined by SEQ ID NOs 1 to 500 and identified at nucleotide positions 61 to 62, and wherein the sensitivity is at least 86% and specificity is at least 77%;
13. at least the CpGs defined by SEQ ID NOs 1 to 1000 and identified at nucleotide positions 61 to 62, and wherein the sensitivity is at least 87% and specificity is at least 77%;
14. at least the CpGs defined by SEQ ID NOs 1 to 1500 and identified at nucleotide positions 61 to 62, and wherein the sensitivity is at least 89% and specificity is at least 76%;
15. at least the CpGs defined by SEQ ID NOs 1 to 2000 and identified at nucleotide positions 61 to 62, and wherein the sensitivity is at least 89% and specificity is at least 76%;
16. at least the CpGs defined by SEQ ID NOs 1 to 2500 and identified at nucleotide positions 61 to 62, and wherein the sensitivity is at least 90% and specificity is at least 75%;
17. at least the CpGs defined by SEQ ID NOs 1 to 3000 and identified at nucleotide positions 61 to 62, and wherein the sensitivity is at least 90% and specificity is at least 73%;
18. at least the CpGs defined by SEQ ID NOs 1 to 3500 and identified at nucleotide positions 61 to 62, and wherein the sensitivity is at least 90% and specificity is at least 73%;
19. at least the CpGs defined by SEQ ID NOs 1 to 4000 and identified at nucleotide positions 61 to 62, and wherein the sensitivity is at least 90% and specificity is at least 76%;
20. at least the CpGs defined by SEQ ID NOs 1 to 4500 and identified at nucleotide positions 61 to 62, and wherein the sensitivity is at least 90% and specificity is at least 74%;
21. at least the CpGs defined by SEQ ID NOs 1 to 5000 and identified at nucleotide positions 61 to 62, and wherein the sensitivity is at least 90% and specificity is at least 75%;
22. at least the CpGs defined by SEQ ID NOs 4501 to 5000 and identified at nucleotide positions 61 to 62, and wherein the sensitivity is at least 87% and specificity is at least 49%;
23. at least the CpGs defined by SEQ ID NOs 4001 to 5000 and identified at nucleotide positions 61 to 62, and wherein the sensitivity is at least 86% and specificity is at least 49%;
24. at least the CpGs defined by SEQ ID NOs 3501 to 5000 and identified at nucleotide positions 61 to 62, and wherein the sensitivity is at least 90% and specificity is at least 51%;
25. at least the CpGs defined by SEQ ID NOs 3001 to 5000 and identified at nucleotide positions 61 to 62, and wherein the sensitivity is at least 89% and specificity is at least 51%;
26. at least the CpGs defined by SEQ ID NOs 2501 to 5000 and identified at nucleotide positions 61 to 62, and wherein the sensitivity is at least 89% and specificity is at least 53%;
27. at least the CpGs defined by SEQ ID NOs 2001 to 5000 and identified at nucleotide positions 61 to 62, and wherein the sensitivity is at least 87% and specificity is at least 52%;
28. at least the CpGs defined by SEQ ID NOs 1501 to 5000 and identified at nucleotide positions 61 to 62, and wherein the sensitivity is at least 87% and specificity is at least 52%;
29. at least the CpGs defined by SEQ ID NOs 1001 to 5000 and identified at nucleotide positions 61 to 62, and wherein the sensitivity is at least 86% and specificity is at least 57%;
30. at least the CpGs defined by SEQ ID NOs 501 to 5000 and identified at nucleotide positions 61 to 62, and wherein the sensitivity is at least 86% and specificity is at least 66%;
31. at least the CpGs defined by SEQ ID NOs 101 to 5000 and identified at nucleotide positions 61 to 62, and wherein the sensitivity is at least 90% and specificity is at least 75%; or
32. at least the CpGs defined by SEQ ID NOs 1 to 5000 and identified at nucleotide positions 61 to 62, and wherein the sensitivity is at least 90% and specificity is at least 75%.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the assays described herein, wherein when the cancer index value for the individual is about −0.167 or more, the individual is assessed as having cancer and/or CIN3, or as having a risk of cancer and/or CIN3 development, or wherein when the cancer index value for the individual is less than about −0.167, the individual is assessed as not having cancer and/or CIN3 or as having a low risk of cancer development and/or CIN3, particularly cervical or endometrial cancer, most preferably cervical cancer, in an individual is based on the WID-CIN-Index. The panel of one or more CpGs used to derive the cancer index value may comprise:
a. at least 500 of the CpGs identified at nucleotide positions 61 to 62 in SEQ ID NOs 1 to 5000, and wherein the sensitivity is at least 80% and the specificity is at least 61%;
b. at least the CpGs defined by SEQ ID NOs 1 to 500 and identified at nucleotide positions 61 to 62, and wherein the sensitivity is at least 77% and specificity is at least 90%;
c. at least the CpGs defined by SEQ ID NOs 1 to 1000 and identified at nucleotide positions 61 to 62, and wherein the sensitivity is at least 72% and specificity is at least 89%;
d. at least the CpGs defined by SEQ ID NOs 1 to 1500 and identified at nucleotide positions 61 to 62, and wherein the sensitivity is at least 78% and specificity is at least 91%.

In any of the assays described herein, wherein when the cancer index value for the individual is about −0.167 or more, the individual is assessed as having cancer and/or CIN3 or as having a high risk of cancer and/or CIN3 development, or wherein when the cancer index value for the individual is less than about −0.167, the individual is assessed as not having cancer and/or CIN3 or as having a low risk of cancer development and/or CIN3, particularly cervical or endometrial cancer, most preferably cervical cancer, in an individual is based on the WID-CIN-Index. The panel of one or more CpGs used to derive the cancer index value may comprise:
1. at least 500 of the CpGs identified at nucleotide positions 61 to 62 in SEQ ID NOs 1 to 5000, and wherein the sensitivity is at least 80% and the specificity is at least 61%;
2. at least 1000 of the CpGs identified at nucleotide positions 61 to 62 in SEQ ID NOs 1 to 5000, and wherein the sensitivity is at least 79% and specificity is at least 59%;
3. at least 1500 of the CpGs identified at nucleotide positions 61 to 62 in SEQ ID NOs 1 to 5000, and wherein the sensitivity is at least 79% and specificity is at least 60%;
4. at least 2000 of the CpGs identified at nucleotide positions 61 to 62 in SEQ ID NOs 1 to 5000, and wherein the sensitivity is at least 80% and specificity is at least 60%;
5. at least 2500 of the CpGs identified at nucleotide positions 61 to 62 in SEQ ID NOs 1 to 5000, and wherein the sensitivity is at least 79% and specificity is at least 63%;
6. at least 3000 of the CpGs identified at nucleotide positions 61 to 62 in SEQ ID NOs 1 to 5000, and wherein the sensitivity is at least 79% and specificity is at least 65%;
7. at least 3500 of the CpGs identified at nucleotide positions 61 to 62 in SEQ ID NOs 1 to 5000, and wherein the sensitivity is at least 76% and specificity is at least 81%;
8. at least 4000 of the CpGs identified at nucleotide positions 61 to 62 in SEQ ID NOs 1 to 5000, and wherein the sensitivity is at least 80% and specificity is at least 70%;
9. at least 4500 of the CpGs identified at nucleotide positions 61 to 62 in SEQ ID NOs 1 to 5000, and wherein the sensitivity is at least 76% and specificity is at least 81%;
10. at least 4900 of the CpGs identified at nucleotide positions 61 to 62 in SEQ ID NOs 1 to 5000, and wherein the sensitivity is at least 74% and specificity is at least 90%; or
11. at least 5000 of the CpGs identified at nucleotide positions 61 to 62 in SEQ ID NOs 1 to 5000, and wherein the sensitivity is at least 78% and specificity is at least 89%.

In any of the assays described herein, wherein when the cancer index value for the individual is about −0.167 or more, the individual is assessed as having cancer and/or CIN3, or as having a risk of cancer and/or CIN3 development, or wherein when the cancer index value for the individual is less than about −0.167, the individual is assessed as not having cancer and/or CIN3 or as having a low risk of cancer development and/or CIN3, particularly cervical or endometrial cancer, most preferably cervical cancer, in an individual is based on the WID-CIN-Index. The panel of one or more CpGs used to derive the cancer index value may comprise:
1. at least the CpGs defined by SEQ ID NOs 1 to 500 and identified at nucleotide positions 61 to 62, and wherein the sensitivity is at least 77% and specificity is at least 90%;
2. at least the CpGs defined by SEQ ID NOs 501 to 1000 and identified at nucleotide positions 61 to 62, and wherein the sensitivity is at least 64% and specificity is at least 92%;
3. at least the CpGs defined by SEQ ID NOs 1001 to 1500 and identified at nucleotide positions 61 to 62, and wherein the sensitivity is at least 76% and specificity is at least 85%;
4. at least the CpGs defined by SEQ ID NOs 1501 to 2000 and identified at nucleotide positions 61 to 62, and wherein the sensitivity is at least 82% and specificity is at least 76%;
5. at least the CpGs defined by SEQ ID NOs 2001 to 2500 and identified at nucleotide positions 61 to 62, and wherein the sensitivity is at least 79% and specificity is at least 77%;
6. at least the CpGs defined by SEQ ID NOs 2501 to 3000 and identified at nucleotide positions 61 to 62, and wherein the sensitivity is at least 80% and specificity is at least 70%;
7. at least the CpGs defined by SEQ ID NOs 3001 to 3500 and identified at nucleotide positions 61 to 62, and wherein the sensitivity is at least 82% and specificity is at least 67%;
8. at least the CpGs defined by SEQ ID NOs 3501 to 4000 and identified at nucleotide positions 61 to 62, and wherein the sensitivity is at least 84% and specificity is at least 62%;
9. at least the CpGs defined by SEQ ID NOs 4001 to 4500 and identified at nucleotide positions 61 to 62, and wherein the sensitivity is at least 77% and specificity is at least 61%;
10. at least the CpGs defined by SEQ ID NOs 4501 to 5000 and identified at nucleotide positions 61 to 62, and wherein the sensitivity is at least 80% and specificity is at least 61%;
11. at least the CpGs defined by SEQ ID NOs 1 to 100 and identified at nucleotide positions 61 to 62, and wherein the sensitivity is at least 75% and specificity is at least 86%;
12. at least the CpGs defined by SEQ ID NOs 1 to 500 and identified at nucleotide positions 61 to 62, and wherein the sensitivity is at least 77% and specificity is at least 90%;
13. at least the CpGs defined by SEQ ID NOs 1 to 1000 and identified at nucleotide positions 61 to 62, and wherein the sensitivity is at least 72% and specificity is at least 89%;
14. at least the CpGs defined by SEQ ID NOs 1 to 1500 and identified at nucleotide positions 61 to 62, and wherein the sensitivity is at least 78% and specificity is at least 91%;
15. at least the CpGs defined by SEQ ID NOs 1 to 2000 and identified at nucleotide positions 61 to 62, and wherein the sensitivity is at least 79% and specificity is at least 90%;
16. at least the CpGs defined by SEQ ID NOs 1 to 2500 and identified at nucleotide positions 61 to 62, and wherein the sensitivity is at least 80% and specificity is at least 89%;
17. at least the CpGs defined by SEQ ID NOs 1 to 3000 and identified at nucleotide positions 61 to 62, and wherein the sensitivity is at least 80% and specificity is at least 89%;
18. at least the CpGs defined by SEQ ID NOs 1 to 3500 and identified at nucleotide positions 61 to 62, and wherein the sensitivity is at least 80% and specificity is at least 89%;
19. at least the CpGs defined by SEQ ID NOs 1 to 4000 and identified at nucleotide positions 61 to 62, and wherein the sensitivity is at least 78% and specificity is at least 90%;
20. at least the CpGs defined by SEQ ID NOs 1 to 4500 and identified at nucleotide positions 61 to 62, and wherein the sensitivity is at least 80% and specificity is at least 89%;
21. at least the CpGs defined by SEQ ID NOs 1 to 5000 and identified at nucleotide positions 61 to 62, and wherein the sensitivity is at least 78% and specificity is at least 89%;
22. at least the CpGs defined by SEQ ID NOs 4501 to 5000 and identified at nucleotide positions 61 to 62, and wherein the sensitivity is at least 80% and specificity is at least 61%;
23. at least the CpGs defined by SEQ ID NOs 4001 to 5000 and identified at nucleotide positions 61 to 62, and wherein the sensitivity is at least 79% and specificity is at least 59%;
24. at least the CpGs defined by SEQ ID NOs 3501 to 5000 and identified at nucleotide positions 61 to 62, and wherein the sensitivity is at least 79% and specificity is at least 60%;
25. at least the CpGs defined by SEQ ID NOs 3001 to 5000 and identified at nucleotide positions 61 to 62, and wherein the sensitivity is at least 80% and specificity is at least 60%;
26. at least the CpGs defined by SEQ ID NOs 2501 to 5000 and identified at nucleotide positions 61 to 62, and wherein the sensitivity is at least 79% and specificity is at least 63%;
27. at least the CpGs defined by SEQ ID NOs 2001 to 5000 and identified at nucleotide positions 61 to 62, and wherein the sensitivity is at least 79% and specificity is at least 65%;
28. at least the CpGs defined by SEQ ID NOs 1501 to 5000 and identified at nucleotide positions 61 to 62, and wherein the sensitivity is at least 80% and specificity is at least 66%;
29. at least the CpGs defined by SEQ ID NOs 1001 to 5000 and identified at nucleotide positions 61 to 62, and wherein the sensitivity is at least 80% and specificity is at least 70%;
30. at least the CpGs defined by SEQ ID NOs 501 to 5000 and identified at nucleotide positions 61 to 62, and wherein the sensitivity is at least 76% and specificity is at least 81%;
31. at least the CpGs defined by SEQ ID NOs 101 to 5000 and identified at nucleotide positions 61 to 62, and wherein the sensitivity is at least 74% and specificity is at least 90%; or
32. at least the CpGs defined by SEQ ID NOs 1 to 5000 and identified at nucleotide positions 61 to 62, and wherein the sensitivity is at least 78% and specificity is at least 89%.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

The ROC data set out in Tables 4, 5 and 6 corresponding to each specified panel of SEQ ID NOs: 1 to 5000 are derived by determining a cancer index value from said panel.

TABLE 4

| | AUC | | | | |
|---|---|---|---|---|---|
| CpG subset | (internal | Sensitivity | Specificity | Sensitivity | Specificity |
| (SEQ ID NO) | validation) | (q = −0.167) | (q = −0.167) | (q = −0.331) | (q = −0.331) |
| 1-500 | 0.92 | 0.77 | 0.9 | 0.86 | 0.77 |
| 501-1000 | 0.9 | 0.64 | 0.92 | 0.84 | 0.77 |
| 1001-1500 | 0.88 | 0.76 | 0.85 | 0.86 | 0.68 |
| 1501-2000 | 0.86 | 0.82 | 0.76 | 0.86 | 0.6 |
| 2001-2500 | 0.86 | 0.79 | 0.77 | 0.9 | 0.61 |
| 2501-3000 | 0.83 | 0.8 | 0.7 | 0.86 | 0.55 |
| 3001-3500 | 0.82 | 0.82 | 0.67 | 0.86 | 0.52 |
| 3501-4000 | 0.82 | 0.84 | 0.62 | 0.86 | 0.53 |
| 4001-4500 | 0.8 | 0.77 | 0.61 | 0.87 | 0.51 |
| 4501-5000 | 0.81 | 0.8 | 0.61 | 0.87 | 0.49 |

TABLE 5

| CpG subset | AUC (internal | Sensitivity | Specificity | Sensitivity | Specificity |
|---|---|---|---|---|---|
| (SEQ ID NO) | validation) | (q = −0.167) | (q = −0.167) | (q = −0.331) | (q = −0.331) |
| 1-100 | 0.9 | 0.75 | 0.86 | 0.86 | 0.77 |
| 1-500 | 0.92 | 0.77 | 0.9 | 0.86 | 0.77 |
| 1-1000 | 0.92 | 0.72 | 0.89 | 0.87 | 0.77 |
| 1-1500 | 0.92 | 0.78 | 0.91 | 0.89 | 0.76 |
| 1-2000 | 0.92 | 0.79 | 0.9 | 0.89 | 0.76 |
| 1-2500 | 0.92 | 0.8 | 0.89 | 0.9 | 0.75 |
| 1-3000 | 0.92 | 0.8 | 0.89 | 0.9 | 0.73 |
| 1-3500 | 0.92 | 0.8 | 0.89 | 0.9 | 0.73 |
| 1-4000 | 0.92 | 0.78 | 0.9 | 0.9 | 0.76 |
| 1-4500 | 0.92 | 0.8 | 0.89 | 0.9 | 0.74 |
| 1-5000 | 0.92 | 0.78 | 0.89 | 0.9 | 0.75 |

TABLE 6

| CpG subset | AUC (internal | Sensitivity | Specificity | Sensitivity | Specificty |
|---|---|---|---|---|---|
| (SEQ ID NO) | validation) | (q = −0.167) | (q = −0.167) | (q = −0.331) | (q = −0.331) |
| 1-5000 | 0.92 | 0.78 | 0.89 | 0.9 | 0.75 |
| 101-5000 | 0.91 | 0.74 | 0.9 | 0.9 | 0.75 |
| 501-5000 | 0.88 | 0.76 | 0.81 | 0.86 | 0.66 |
| 1001-5000 | 0.84 | 0.8 | 0.7 | 0.86 | 0.57 |
| 1501-5000 | 0.82 | 0.8 | 0.66 | 0.87 | 0.52 |
| 2001-5000 | 0.82 | 0.79 | 0.65 | 0.87 | 0.52 |
| 2501-5000 | 0.82 | 0.79 | 0.63 | 0.89 | 0.53 |
| 3001-5000 | 0.81 | 0.8 | 0.6 | 0.89 | 0.51 |
| 3501-5000 | 0.82 | 0.79 | 0.6 | 0.9 | 0.51 |
| 4001-5000 | 0.8 | 0.79 | 0.59 | 0.86 | 0.49 |
| 4501-5000 | 0.81 | 0.8 | 0.61 | 0.87 | 0.49 |

The predicting of the presence, absence, or development of cancer in an individual may particularly involve determining the mean β-value across any panel of one or more CpGs defined herein. A threshold mean β-value may be applied in order to stratify an individual as having or not having cancer, or of having a high or low risk of CIN3 and/or cancer development, preferably wherein the cancer is cervical or endometrial cancer, more preferably wherein the cancer is cervical cancer.

In any of the assays described herein, wherein:
4. when the cancer index for the individual is about 0.016 or more the individual is classified as having CIN3 and/or cancer or as having a high risk of CIN3 and/or cancer development; or
5. when the cancer index for the individual is less than about 0.016 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.025 or more the individual is classified as having CIN3 and/or cancer or as having a high risk of CIN3 and/or cancer development; or
b. when the cancer index for the individual is less than about 0.025 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.050 or more the individual is classified as having CIN3 and/or cancer or as having a high risk of CIN3 and/or cancer development; or
b. when the cancer index for the individual is less than about 0.050 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.075 or more the individual is classified as having CIN3 and/or cancer or as having a high risk of CIN3 and/or cancer development; or
b. when the cancer index for the individual is less than about 0.075 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.100 or more the individual is classified as having CIN3 and/or cancer or as having a high risk of CIN3 and/or cancer development; or
b. when the cancer index for the individual is less than about 0.100 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.125 or more the individual is classified as having CIN3 and/or cancer or as having a high risk of CIN3 and/or cancer development; or
b. when the cancer index for the individual is less than about 0.125 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.150 or more the individual is classified as having CIN3 and/or cancer or as having a high risk of CIN3 and/or cancer development; or
b. when the cancer index for the individual is less than about 0.150 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.175 or more the individual is classified as having CIN3 and/or cancer or as having a high risk of CIN3 and/or cancer development; or
b. when the cancer index for the individual is less than about 0.175 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.200 or more the individual is classified as having CIN3 and/or cancer or as having a high risk of CIN3 and/or cancer development; or
b. when the cancer index for the individual is less than about 0.200 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.225 or more the individual is classified as having CIN3 and/or cancer or as having a high risk of CIN3 and/or cancer development; or
b. when the cancer index for the individual is less than about 0.225 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein, wherein:
a. when the cancer index for the individual is about 0.250 or more the individual is classified as having CIN3 and/or cancer or as having a high risk of CIN3 and/or cancer development; or
b. when the cancer index for the individual is less than about 0.250 the individual is classified as not having CIN3 and/or cancer;
preferably wherein the assay has a specificity of 75% or more, more preferably wherein the assay comprises determining mean β-values for each CpG in the panel of one or more CpGs.

In any of the assays described herein:
1. CpGs whose cancer index value is determined are located within at least DMR 1 defined by SEQ ID NO: 5001, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpGs from DMR 1, and more preferably wherein the cancer index value is the mean β-value for the CpGs denoted by [[CG]] in SEQ ID NO: 5001;
2. CpGs whose cancer index value is determined are located within at least DMR 1 defined by SEQ ID NO: 5001, and wherein when the cancer index for the individual is less than about 0.023 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least ten CpGs from DMR 1, and more preferably wherein the cancer index value is the mean β-value for the CpGs denoted by [[CG]] in SEQ ID NO: 5001;
3. CpGs whose cancer index value is determined are located within at least DMR 2 defined by SEQ ID NO: 5002, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 2, and more preferably wherein the cancer index value is the mean β-value for the CpGs denoted by [[CG]] in SEQ ID NO: 5002;
4. CpGs whose cancer index value is determined are located within at least DMR 2 defined by SEQ ID NO: 5002, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpGs from DMR 2, and more preferably wherein the cancer index value is the mean β-value for the CpGs denoted by [[CG]] in SEQ ID NO: 5002;
5. CpGs whose cancer index value is determined are located within at least DMR 3 defined by SEQ ID NO: 5003, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 3, and more preferably wherein the cancer index value is the mean β-value for the CpGs denoted by [[CG]] in SEQ ID NO: 5003;
6. CpGs whose cancer index value is determined are located within at least DMR 3 defined by SEQ ID NO: 5003, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 3, and more preferably wherein the cancer index value is the mean β-value for the CpGs denoted by [[CG]] in SEQ ID NO: 5003;
7. CpGs whose cancer index value is determined are located within at least DMR 4 defined by SEQ ID NO: 5004, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, DMR 4, and more preferably wherein the cancer index value is the mean β-value for the CpGs denoted by [[CG]] in SEQ ID NO: 5004;
8. CpGs whose cancer index value is determined are located within at least DMR 4 defined by SEQ ID NO: 5004, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 4, and more preferably wherein the cancer index value is the mean β-value for the CpGs denoted by [[CG]] in SEQ ID NO: 5004;
9. CpGs whose cancer index value is determined are located within at least DMR 5 defined by SEQ ID NO: 5005, and wherein when the cancer index for the individual is about 0.023 or more the individual is classified as having CIN3 and/or cancer or as having a high risk of CIN3 and/or cancer development, and wherein the sensitivity of the assay is at least 85.00% and the specificity of the assay is at least 75.00%, preferably wherein the panel of one or more CpGs comprises at least ten CpG from DMR 5, and more preferably wherein the cancer index value is the mean β-value for the CpGs denoted by [[CG]] in SEQ ID NO: 5005;
10. CpGs whose cancer index value is determined are located within at least DMR 5 defined by SEQ ID NO: 5005, and wherein when the cancer index for the individual is less than about 0.023 the individual is classified as not having CIN3 and/or cancer or as having a low risk of CIN3 and/or cancer development, and wherein the sensitivity of the assay is at least 85.00% and the specificity of the assay is at least 75.00%, preferably wherein the panel of one or more CpGs comprises at least ten CpG from DMR 5, and more preferably wherein the cancer index value is the mean β-value for the CpGs denoted by [[CG]] in SEQ ID NO: 5005;
11. CpGs whose cancer index value is determined are located within at least DMR 6 defined by SEQ ID NO: 5006, and wherein when the cancer index for the individual is about 0.027 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least nine CpG from DMR 6, and more preferably wherein the cancer index value is the mean β-value for the CpGs denoted by [[CG]] in SEQ ID NO: 5006;
12. CpGs whose cancer index value is determined are located within at least DMR 6 defined by SEQ ID NO: 5006, and wherein when the cancer index for the individual is less than about 0.027 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least nine CpG from DMR 6, and more preferably wherein the cancer index value is the mean β-value for the CpGs denoted by [[CG]] in SEQ ID NO: 5006;
13. CpGs whose cancer index value is determined are located within at least DMR 7 defined by SEQ ID NO: 5007, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 7, and more preferably wherein the cancer index value is the mean β-value for the CpGs denoted by [[CG]] in SEQ ID NO: 5007;
14. CpGs whose cancer index value is determined are located within at least DMR 7 defined by SEQ ID NO: 5007, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 7, and more preferably wherein the cancer index value is the mean β-value for the CpGs denoted by [[CG]] in SEQ ID NO: 5007;
15. CpGs whose cancer index value is determined are located within at least DMR 8 defined by SEQ ID NO: 5008, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 8, and more preferably wherein the cancer index value is the mean β-value for the CpGs denoted by [[CG]] in SEQ ID NO: 5008;
16. CpGs whose cancer index value is determined are located within at least DMR 8 defined by SEQ ID NO: 5008, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 8, and more preferably wherein the cancer index value is the mean β-value for the CpGs denoted by [[CG]] in SEQ ID NO: 5008;
17. CpGs whose cancer index value is determined are located within at least DMR 9 defined by SEQ ID NO: 5009, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 9, and more preferably wherein the cancer index value is the mean β-value for the CpGs denoted by [[CG]] in SEQ ID NO: 5009;
18. CpGs whose cancer index value is determined are located within at least DMR 9 defined by SEQ ID NO: 5009, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 9, and more preferably wherein the cancer index value is the mean β-value for the CpGs denoted by [[CG]] in SEQ ID NO: 5009;
19. CpGs whose cancer index value is determined are located within at least DMR 10 defined by SEQ ID NO: 5010, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 10, and more preferably wherein the cancer index value is the mean β-value for the CpGs denoted by [[CG]] in SEQ ID NO: 5010;
20. CpGs whose cancer index value is determined are located within at least DMR 10 defined by SEQ ID NO: 5010, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 10, and more preferably wherein the cancer index value is the mean β-value for the CpGs denoted by [[CG]] in SEQ ID NO: 5010;
21. CpGs whose cancer index value is determined are located within at least DMR 11 defined by SEQ ID NO: 5011, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 11, and more preferably wherein the cancer index value is the mean β-value for the CpGs denoted by [[CG]] in SEQ ID NO: 5011;
22. CpGs whose cancer index value is determined are located within at least DMR 11 defined by SEQ ID NO: 5011, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 11, and more preferably wherein the cancer index value is the mean β-value for the CpGs denoted by [[CG]] in SEQ ID NO: 5011;
23. CpGs whose cancer index value is determined are located within at least DMR 12 defined by SEQ ID NO: 5012, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 12, and more preferably wherein the cancer index value is the mean β-value for the CpGs denoted by [[CG]] in SEQ ID NO: 5012;
24. CpGs whose cancer index value is determined are located within at least DMR 12 defined by SEQ ID NO: 5012, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 12, and more preferably wherein the cancer index value is the mean β-value for the CpGs denoted by [[CG]] in SEQ ID NO: 5012;
25. CpGs whose cancer index value is determined are located within at least DMR 13 defined by SEQ ID NO: 5013, and wherein when the cancer index for the individual is about 0.027 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 13, and more preferably wherein the cancer index value is the mean β-value for the CpGs denoted by [[CG]] in SEQ ID NO: 5013;
26. CpGs whose cancer index value is determined are located within at least DMR 13 defined by SEQ ID NO: 5013, and wherein when the cancer index for the individual is less than about 0.027 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least nine CpG from DMR 13, and more preferably wherein the cancer index value is the mean β-value for the CpGs denoted by [[CG]] in SEQ ID NO: 5013;
27. CpGs whose cancer index value is determined are located within at least DMR 14 defined by SEQ ID NO: 5014, and wherein when the cancer index for the individual is about 0.02 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least two CpG from DMR 14, and more preferably wherein the cancer index value is the mean β-value for the CpGs denoted by [[CG]] in SEQ ID NO: 5014;
28. CpGs whose cancer index value is determined are located within at least DMR 14 defined by SEQ ID NO: 5014, and wherein when the cancer index for the individual is less than about 0.02 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least two CpG from DMR 14, and more preferably wherein the cancer index value is the mean β-value for the CpGs denoted by [[CG]] in SEQ ID NO: 5014;
29. CpGs whose cancer index value is determined are located within at least DMR 15 defined by SEQ ID NO: 5015, and wherein when the cancer index for the individual is about 0.026 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least six CpG from DMR 15, and more preferably wherein the cancer index value is the mean β-value for the CpGs denoted by [[CG]] in SEQ ID NO: 5015;
30. CpGs whose cancer index value is determined are located within at least DMR 15 defined by SEQ ID NO: 5015, and wherein when the cancer index for the individual is less than about 0.026 the individual is classified as not having CIN3 and/or cancer or as having a low risk of CIN3 and/or cancer development, and wherein the sensitivity of the assay is at least 83.00% and the specificity of the assay is at least 75.00%, DMR 15, and more preferably wherein the cancer index value is the mean β-value for the CpGs denoted by [[CG]] in SEQ ID NO: 5015;
31. CpGs whose cancer index value is determined are located within at least DMR 16 defined by SEQ ID NO: 5016, and wherein when the cancer index for the individual is about 0.026 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least six CpG from DMR 16, and more preferably wherein the cancer index value is the mean β-value for the CpGs denoted by [[CG]] in SEQ ID NO: 5016;
32. CpGs whose cancer index value is determined are located within at least DMR 16 defined by SEQ ID NO: 5016, and wherein when the cancer index for the individual is less than about 0.026 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least six CpG from DMR 16, and more preferably wherein the cancer index value is the mean β-value for the CpGs denoted by [[CG]] in SEQ ID NO: 5016;
33. CpGs whose cancer index value is determined are located within at least DMR 17 defined by SEQ ID NO: 5017, and wherein when the cancer index for the individual is about 0.026 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least six CpG from DMR 17, and more preferably wherein the cancer index value is the mean β-value for the CpGs denoted by [[CG]] in SEQ ID NO: 5017;
34. CpGs whose cancer index value is determined are located within at least DMR 17 defined by SEQ ID NO: 5017, and wherein when the cancer index for the individual is less than about 0.026 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least six CpG from DMR 17, and more preferably wherein the cancer index value is the mean β-value for the CpGs denoted by [[CG]] in SEQ ID NO: 5017;
35. CpGs whose cancer index value is determined are located within at least DMR 18 defined by SEQ ID NO: 5018, and wherein when the cancer index for the individual is about 0.025 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least fifteen CpG from DMR 18, and more preferably wherein the cancer index value is the mean β-value for the CpGs denoted by [[CG]] in SEQ ID NO: 5018;
36. CpGs whose cancer index value is determined are located within at least DMR 18 defined by SEQ ID NO: 5018, and wherein when the cancer index for the individual is less than about 0.025 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least fifteen CpG from DMR 18, and more preferably wherein the cancer index value is the mean β-value for the CpGs denoted by [[CG]] in SEQ ID NO: 5018;
37. CpGs whose cancer index value is determined are located within at least DMR 19 defined by SEQ ID NO: 5019, and wherein when the cancer index for the individual is about 0.025 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least fifteen CpG from DMR 19, and more preferably wherein the cancer index value is the mean β-value for the CpGs denoted by [[CG]] in SEQ ID NO: 5019;
38. CpGs whose cancer index value is determined are located within at least DMR 19 defined by SEQ ID NO: 5019, and wherein when the cancer index for the individual is less than about 0.025 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least fifteen CpG from DMR 19, and more preferably wherein the cancer index value is the mean β-value for the CpGs denoted by [[CG]] in SEQ ID NO: 5019;
39. CpGs whose cancer index value is determined are located within at least DMR 20 defined by SEQ ID NO: 5020, and wherein when the cancer index for the individual is about 0.028 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least ten CpG from DMR 20, and more preferably wherein the cancer index value is the mean β-value for the CpGs denoted by [[CG]] in SEQ ID NO: 5020;
40. CpGs whose cancer index value is determined are located within at least DMR 20 defined by SEQ ID NO: 5020, and wherein when the cancer index for the individual is less than about 0.028 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least ten CpG from DMR 20, and more preferably wherein the cancer index value is the mean β-value for the CpGs denoted by [[CG]] in SEQ ID NO: 5020;
41. CpGs whose cancer index value is determined are located within at least DMR 21 defined by SEQ ID NO: 5021, and wherein when the cancer index for the individual is about 0.03 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least seven CpG from DMR 21, and more preferably wherein the cancer index value is the mean β-value for the CpGs denoted by [[CG]] in SEQ ID NO: 5021;
42. CpGs whose cancer index value is determined are located within at least DMR 21 defined by SEQ ID NO: 5021, and wherein when the cancer index for the individual is less than about 0.03 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least seven CpG from DMR 21, and more preferably wherein the cancer index value is the mean β-value for the CpGs denoted by [[CG]] in SEQ ID NO: 5021;
43. CpGs whose cancer index value is determined are located within at least DMR 22 defined by SEQ ID NO: 5022, and wherein when the cancer index for the individual is about 0.019 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least four CpG from DMR 22, and more preferably wherein the cancer index value is the mean β-value for the CpGs denoted by [[CG]] in SEQ ID NO: 5022;
44. CpGs whose cancer index value is determined are located within at least DMR 22 defined by SEQ ID NO: 5022, and wherein when the cancer index for the individual is less than about 0.019 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least four CpG from DMR 22, and more preferably wherein the cancer index value is the mean β-value for the CpGs denoted by [[CG]] in SEQ ID NO: 5022;
45. CpGs whose cancer index value is determined are located within at least DMR 23 defined by SEQ ID NO: 5023, and wherein when the cancer index for the individual is about 0.029 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least six CpG from DMR 23, and more preferably wherein the cancer index value is the mean β-value for the CpGs denoted by [[CG]] in SEQ ID NO: 5023;
46. CpGs whose cancer index value is determined are located within at least DMR 23 defined by SEQ ID NO: 5023, and wherein when the cancer index for the individual is less than about 0.029 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least six CpG from DMR 23, and more preferably wherein the cancer index value is the mean β-value for the CpGs denoted by [[CG]] in SEQ ID NO: 5023;
47. CpGs whose cancer index value is determined are located within at least DMR 24 defined by SEQ ID NO: 5024, and wherein when the cancer index for the individual is about 0.039 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least three CpG from DMR 24, and more preferably wherein the cancer index value is the mean β-value for the CpGs denoted by [[CG]] in SEQ ID NO: 5024;
48. CpGs whose cancer index value is determined are located within at least DMR 24 defined by SEQ ID NO: 5024, and wherein when the cancer index for the individual is less than about 0.039 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least three CpG from DMR 24, and more preferably wherein the cancer index value is the mean β-value for the CpGs denoted by [[CG]] in SEQ ID NO: 5024;
49. CpGs whose cancer index value is determined are located within at least DMR 25 defined by SEQ ID NO: 5025, and wherein when the cancer index for the individual is about 0.048 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least three CpG from DMR 25, and more preferably wherein the cancer index value is the mean β-value for the CpGs denoted by [[CG]] in SEQ ID NO: 5025;
50. CpGs whose cancer index value is determined are located within at least DMR 25 defined by SEQ ID NO: 5025, and wherein when the cancer index for the individual is less than about 0.048 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least three CpG from DMR 25, and more preferably wherein the cancer index value is the mean β-value for the CpGs denoted by [[CG]] in SEQ ID NO: 5025;
51. CpGs whose cancer index value is determined are located within at least DMR 26 defined by SEQ ID NO: 5026, and wherein when the cancer index for the individual is about 0.048 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least three CpG from DMR 26, and more preferably wherein the cancer index value is the mean β-value for the CpGs denoted by [[CG]] in SEQ ID NO: 5026;
52. CpGs whose cancer index value is determined are located within at least DMR 26 defined by SEQ ID NO: 5026, and wherein when the cancer index for the individual is less than about 0.048 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least three CpG from DMR 26, and more preferably wherein the cancer index value is the mean β-value for the CpGs denoted by [[CG]] in SEQ ID NO: 5026;
53. CpGs whose cancer index value is determined are located within at least DMR 27 defined by SEQ ID NO: 5027, and wherein when the cancer index for the individual is about 0.022 or more the individual is classified as having CIN3 and/or cancer or as having a high risk of CIN3 and/or cancer development, and wherein the sensitivity of the assay is at least 79.00% and the specificity of the assay is at least 75.00%, DMR 27, and more preferably wherein the cancer index value is the mean β-value for the CpGs denoted by [[CG]] in SEQ ID NO: 5027;
54. CpGs whose cancer index value is determined are located within at least DMR 27 defined by SEQ ID NO: 5027, and wherein when the cancer index for the individual is less than about 0.022 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least five CpG from DMR 27, and more preferably wherein the cancer index value is the mean β-value for the CpGs denoted by [[CG]] in SEQ ID NO: 5027;
55. CpGs whose cancer index value is determined are located within at least DMR 28 defined by SEQ ID NO: 5028, and wherein when the cancer index for the individual is about 0.048 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least three CpG from DMR 28, and more preferably wherein the cancer index value is the mean β-value for the CpGs denoted by [[CG]] in SEQ ID NO: 5028;
56. CpGs whose cancer index value is determined are located within at least DMR 28 defined by SEQ ID NO: 5028, and wherein when the cancer index for the individual is less than about 0.048 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least three CpG from DMR 28, and more preferably wherein the cancer index value is the mean β-value for the CpGs denoted by [[CG]] in SEQ ID NO: 5028;
57. CpGs whose cancer index value is determined are located within at least DMR 29 defined by SEQ ID NO: 5029, and wherein when the cancer index for the individual is about 0.022 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least five CpG from DMR 29, and more preferably wherein the cancer index value is the mean β-value for the CpGs denoted by [[CG]] in SEQ ID NO: 5029;
58. CpGs whose cancer index value is determined are located within at least DMR 29 defined by SEQ ID NO: 5029, and wherein when the cancer index for the individual is less than about 0.022 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least five CpG from DMR 29, and more preferably wherein the cancer index value is the mean β-value for the CpGs denoted by [[CG]] in SEQ ID NO: 5029;
59. CpGs whose cancer index value is determined are located within at least DMR 30 defined by SEQ ID NO: 5030, and wherein when the cancer index for the individual is about 0.034 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least two CpG from DMR 30, and more preferably wherein the cancer index value is the mean β-value for the CpGs denoted by [[CG]] in SEQ ID NO: 5030;
60. CpGs whose cancer index value is determined are located within at least DMR 30 defined by SEQ ID NO: 5030, and wherein when the cancer index for the individual is less than about 0.034 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least two CpG from DMR 30, and more preferably wherein the cancer index value is the mean β-value for the CpGs denoted by [[CG]] in SEQ ID NO: 5030;
61. CpGs whose cancer index value is determined are located within at least DMR 31 defined by SEQ ID NO: 5031, and wherein when the cancer index for the individual is about 0.03 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least ten CpG from DMR 31, and more preferably wherein the cancer index value is the mean β-value for the CpGs denoted by [[CG]] in SEQ ID NO: 5031;
62. CpGs whose cancer index value is determined are located within at least DMR 31 defined by SEQ ID NO: 5031, and wherein when the cancer index for the individual is less than about 0.03 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least ten CpG from DMR 31, and more preferably wherein the cancer index value is the mean β-value for the CpGs denoted by [[CG]] in SEQ ID NO: 5031;
63. CpGs whose cancer index value is determined are located within at least DMR 32 defined by SEQ ID NO: 5032, and wherein when the cancer index for the individual is about 0.027 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least five CpG from DMR 32, and more preferably wherein the cancer index value is the mean β-value for the CpGs denoted by [[CG]] in SEQ ID NO: 5032;
64. CpGs whose cancer index value is determined are located within at least DMR 32 defined by SEQ ID NO: 5032, and wherein when the cancer index for the individual is less than about 0.027 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least five CpG from DMR 32, and more preferably wherein the cancer index value is the mean β-value for the CpGs denoted by [[CG]] in SEQ ID NO: 5032;
65. CpGs whose cancer index value is determined are located within at least DMR 33 defined by SEQ ID NO: 5033, and wherein when the cancer index for the individual is about 0.027 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least five CpG from DMR 33, and more preferably wherein the cancer index value is the mean β-value for the CpGs denoted by [[CG]] in SEQ ID NO: 5033;
66. CpGs whose cancer index value is determined are located within at least DMR 33 defined by SEQ ID NO: 5033, and wherein when the cancer index for the individual is less than about 0.027 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least five CpG from DMR 33, and more preferably wherein the cancer index value is the mean β-value for the CpGs denoted by [[CG]] in SEQ ID NO: 5033;
67. CpGs whose cancer index value is determined are located within at least DMR 34 defined by SEQ ID NO: 5034, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 34, and more preferably wherein the cancer index value is the mean β-value for the CpGs denoted by [[CG]] in SEQ ID NO: 5034;
68. CpGs whose cancer index value is determined are located within at least DMR 34 defined by SEQ ID NO: 5034, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 34, and more preferably wherein the cancer index value is the mean β-value for the CpGs denoted by [[CG]] in SEQ ID NO: 5034;
69. CpGs whose cancer index value is determined are located within at least DMR 35 defined by SEQ ID NO: 5035, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 35, and more preferably wherein the cancer index value is the mean β-value for the CpGs denoted by [[CG]] in SEQ ID NO: 5035;
70. CpGs whose cancer index value is determined are located within at least DMR 35 defined by SEQ ID NO: 5035, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 35, and more preferably wherein the cancer index value is the mean β-value for the CpGs denoted by [[CG]] in SEQ ID NO: 5035;
71. CpGs whose cancer index value is determined are located within at least DMR 36 defined by SEQ ID NO: 5036, and wherein when the cancer index for the individual is about 0.026 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least ten CpG from DMR 36, and more preferably wherein the cancer index value is the mean β-value for the CpGs denoted by [[CG]] in SEQ ID NO: 5036;
72. CpGs whose cancer index value is determined are located within at least DMR 36 defined by SEQ ID NO: 5036, and wherein when the cancer index for the individual is less than about 0.026 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least ten CpG from DMR 36, and more preferably wherein the cancer index value is the mean β-value for the CpGs denoted by [[CG]] in SEQ ID NO: 5036;
73. CpGs whose cancer index value is determined are located within at least DMR 37 defined by SEQ ID NO: 5037, and wherein when the cancer index for the individual is about 0.026 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least ten CpG from DMR 37, and more preferably wherein the cancer index value is the mean β-value for the CpGs denoted by [[CG]] in SEQ ID NO: 5037;
74. CpGs whose cancer index value is determined are located within at least DMR 37 defined by SEQ ID NO: 5037, and wherein when the cancer index for the individual is less than about 0.026 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least ten CpG from DMR 37, and more preferably wherein the cancer index value is the mean β-value for the CpGs denoted by [[CG]] in SEQ ID NO: 5037;
75. CpGs whose cancer index value is determined are located within at least DMR 38 defined by SEQ ID NO: 5038, and wherein when the cancer index for the individual is about 0.026 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least ten CpG from DMR 38, and more preferably wherein the cancer index value is the mean β-value for the CpGs denoted by [[CG]] in SEQ ID NO: 5038;
76. CpGs whose cancer index value is determined are located within at least DMR 38 defined by SEQ ID NO: 5038, and wherein when the cancer index for the individual is less than about 0.026 the individual is classified as not having CIN3 and/or cancer or as having a low risk of CIN3 and/or cancer development, and wherein the sensitivity of the assay is at least 80.00% and the specificity of the assay is at least 75.00%, DMR 38, and more preferably wherein the cancer index value is the mean β-value for the CpGs denoted by [[CG]] in SEQ ID NO: 5038;
77. CpGs whose cancer index value is determined are located within at least DMR 39 defined by SEQ ID NO: 5039, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 39, and more preferably wherein the cancer index value is the mean β-value for the CpGs denoted by [[CG]] in SEQ ID NO: 5039;
78. CpGs whose cancer index value is determined are located within at least DMR 39 defined by SEQ ID NO: 5039, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 39, and more preferably wherein the cancer index value is the mean β-value for the CpGs denoted by [[CG]] in SEQ ID NO: 5039;
79. CpGs whose cancer index value is determined are located within at least DMR 40 defined by SEQ ID NO: 5040, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 40, and more preferably wherein the cancer index value is the mean β-value for the CpGs denoted by [[CG]] in SEQ ID NO: 5040;
80. CpGs whose cancer index value is determined are located within at least DMR 40 defined by SEQ ID NO: 5040, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 40, and more preferably wherein the cancer index value is the mean β-value for the CpGs denoted by [[CG]] in SEQ ID NO: 5040;
81. CpGs whose cancer index value is determined are located within at least DMR 41 defined by SEQ ID NO: 5041, and wherein when the cancer index for the individual is about 0.026 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least ten CpG from DMR 41, and more preferably wherein the cancer index value is the mean β-value for the CpGs denoted by [[CG]] in SEQ ID NO: 5041;
82. CpGs whose cancer index value is determined are located within at least DMR 41 defined by SEQ ID NO: 5041, and wherein when the cancer index for the individual is less than about 0.026 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least ten CpG from DMR 41, and more preferably wherein the cancer index value is the mean β-value for the CpGs denoted by [[CG]] in SEQ ID NO: 5041;
83. CpGs whose cancer index value is determined are located within at least DMR 42 defined by SEQ ID NO: 5042, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 42, and more preferably wherein the cancer index value is the mean β-value for the CpGs denoted by [[CG]] in SEQ ID NO: 5042;
84. CpGs whose cancer index value is determined are located within at least DMR 42 defined by SEQ ID NO: 5042, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 42, and more preferably wherein the cancer index value is the mean β-value for the CpGs denoted by [[CG]] in SEQ ID NO: 5042;
85. CpGs whose cancer index value is determined are located within at least DMR 43 defined by SEQ ID NO: 5043, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 43, and more preferably wherein the cancer index value is the mean β-value for the CpGs denoted by [[CG]] in SEQ ID NO: 5043;
86. CpGs whose cancer index value is determined are located within at least DMR 43 defined by SEQ ID NO: 5043, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 43, and more preferably wherein the cancer index value is the mean β-value for the CpGs denoted by [[CG]] in SEQ ID NO: 5043;
87. CpGs whose cancer index value is determined are located within at least DMR 44 defined by SEQ ID NO: 5044, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 44, and more preferably wherein the cancer index value is the mean β-value for the CpGs denoted by [[CG]] in SEQ ID NO: 5044;
88. CpGs whose cancer index value is determined are located within at least DMR 44 defined by SEQ ID NO: 5044, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 44, and more preferably wherein the cancer index value is the mean β-value for the CpGs denoted by [[CG]] in SEQ ID NO: 5044;
89. CpGs whose cancer index value is determined are located within at least DMR 45 defined by SEQ ID NO: 5045, and wherein when the cancer index for the individual is about 0.041 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least nine CpG from DMR 45, and more preferably wherein the cancer index value is the mean β-value for the CpGs denoted by [[CG]] in SEQ ID NO: 5045;
90. CpGs whose cancer index value is determined are located within at least DMR 45 defined by SEQ ID NO: 5045, and wherein when the cancer index for the individual is less than about 0.041 the individual is classified as not having CIN3 and/or cancer or as having a low risk of CIN3 and/or cancer development, and wherein the sensitivity of preferably wherein the panel of one or more CpGs comprises at least nine CpG from DMR 45, and more preferably wherein the cancer index value is the mean β-value for the CpGs denoted by [[CG]] in SEQ ID NO: 5045;
91. CpGs whose cancer index value is determined are located within at least DMR 46 defined by SEQ ID NO: 5046, and wherein when the cancer index for the individual is about 0.038 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 46, and more preferably wherein the cancer index value is the mean β-value for the CpGs denoted by [[CG]] in SEQ ID NO: 5046;
92. CpGs whose cancer index value is determined are located within at least DMR 46 defined by SEQ ID NO: 5046, and wherein when the cancer index for the individual is less than about 0.038 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 46, and more preferably wherein the cancer index value is the mean β-value for the CpGs denoted by [[CG]] in SEQ ID NO: 5046;
93. CpGs whose cancer index value is determined are located within at least DMR 47 defined by SEQ ID NO: 5047, and wherein when the cancer index for the individual is about 0.029 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eleven CpG from DMR 47, and more preferably wherein the cancer index value is the mean β-value for the CpGs denoted by [[CG]] in SEQ ID NO: 5047;
94. CpGs whose cancer index value is determined are located within at least DMR 47 defined by SEQ ID NO: 5047, and wherein when the cancer index for the individual is less than about 0.029 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eleven CpG from DMR 47, and more preferably wherein the cancer index value is the mean β-value for the CpGs denoted by [[CG]] in SEQ ID NO: 5047;
95. CpGs whose cancer index value is determined are located within at least DMR 48 defined by SEQ ID NO: 5048, and wherein when the cancer index for the individual is about 0.028 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least six CpG from DMR 48, and more preferably wherein the cancer index value is the mean β-value for the CpGs denoted by [[CG]] in SEQ ID NO: 5048;
96. CpGs whose cancer index value is determined are located within at least DMR 48 defined by SEQ ID NO: 5048, and wherein when the cancer index for the individual is less than about 0.028 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least six CpG from DMR 48, and more preferably wherein the cancer index value is the mean β-value for the CpGs denoted by [[CG]] in SEQ ID NO: 5048;
97. CpGs whose cancer index value is determined are located within at least DMR 49 defined by SEQ ID NO: 5049, and wherein when the cancer index for the individual is about 0.028 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least six CpG from DMR 49, and more preferably wherein the cancer index value is the mean β-value for the CpGs denoted by [[CG]] in SEQ ID NO: 5049;
98. CpGs whose cancer index value is determined are located within at least DMR 49 defined by SEQ ID NO: 5049, and wherein when the cancer index for the individual is less than about 0.028 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least six CpG from DMR 49, and more preferably wherein the cancer index value is the mean β-value for the CpGs denoted by [[CG]] in SEQ ID NO: 5049;
99. CpGs whose cancer index value is determined are located within at least DMR 50 defined by SEQ ID NO: 5050, and wherein when the cancer index for the individual is about 0.028 or more the individual is classified as having CIN3 and/or cancer or as having a high risk of CIN3 and/or cancer development, and wherein the sensitivity of the assay is at least 83.00% and the specificity of the assay is at least 75.00%, DMR 50, and more preferably wherein the cancer index value is the mean β-value for the CpGs denoted by [[CG]] in SEQ ID NO: 5050;
100. CpGs whose cancer index value is determined are located within at least DMR 50 defined by SEQ ID NO: 5050, and wherein when the cancer index for the individual is less than about 0.028 the individual is classified as not having CIN3 and/or cancer or as having a low risk of CIN3 and/or cancer development, and wherein the sensitivity of the assay is at least 83.00% and the specificity of the assay is at least 75.00%, preferably wherein the panel of one or more CpGs comprises at least six CpG from DMR 50, and more preferably wherein the cancer index value is the mean β-value for the CpGs denoted by [[CG]] in SEQ ID NO: 5050;
101. CpGs whose cancer index value is determined are located within at least DMR 51 defined by SEQ ID NO: 5051, and wherein when the cancer index for the individual is about 0.034 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least six CpG from DMR 51, and more preferably wherein the cancer index value is the mean β-value for the CpGs denoted by [[CG]] in SEQ ID NO: 5051;
102. CpGs whose cancer index value is determined are located within at least DMR 51 defined by SEQ ID NO: 5051, and wherein when the cancer index for the individual is less than about 0.034 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least six CpG from DMR 51, and more preferably wherein the cancer index value is the mean β-value for the CpGs denoted by [[CG]] in SEQ ID NO: 5051;
103. CpGs whose cancer index value is determined are located within at least DMR 52 defined by SEQ ID NO: 5052, and wherein when the cancer index for the individual is about 0.034 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least six CpG from DMR 52, and more preferably wherein the cancer index value is the mean β-value for the CpGs denoted by [[CG]] in SEQ ID NO: 5052;
104. CpGs whose cancer index value is determined are located within at least DMR 52 defined by SEQ ID NO: 5052, and wherein when the cancer index for the individual is less than about 0.034 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least six CpG from DMR 52, and more preferably wherein the cancer index value is the mean β-value for the CpGs denoted by [[CG]] in SEQ ID NO: 5052;
105. CpGs whose cancer index value is determined are located within at least DMR 53 defined by SEQ ID NO: 5053, and wherein when the cancer index for the individual is about 0.036 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least three CpG from DMR 53, and more preferably wherein the cancer index value is the mean β-value for the CpGs denoted by [[CG]] in SEQ ID NO: 5053;
106. CpGs whose cancer index value is determined are located within at least DMR 53 defined by SEQ ID NO: 5053, and wherein when the cancer index for the individual is less than about 0.036 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least three CpG from DMR 53, and more preferably wherein the cancer index value is the mean β-value for the CpGs denoted by [[CG]] in SEQ ID NO: 5053;
107. CpGs whose cancer index value is determined are located within at least DMR 54 defined by SEQ ID NO: 5054, and wherein when the cancer index for the individual is about 0.036 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least three CpG from DMR 54, and more preferably wherein the cancer index value is the mean β-value for the CpGs denoted by [[CG]] in SEQ ID NO: 5054;
108. CpGs whose cancer index value is determined are located within at least DMR 54 defined by SEQ ID NO: 5054, and wherein when the cancer index for the individual is less than about 0.036 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least three CpG from DMR 54, and more preferably wherein the cancer index value is the mean β-value for the CpGs denoted by [[CG]] in SEQ ID NO: 5054;
109. CpGs whose cancer index value is determined are located within at least DMR 55 defined by SEQ ID NO: 5055, and wherein when the cancer index for the individual is about 0.05 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least two CpG from DMR 55, and more preferably wherein the cancer index value is the mean β-value for the CpGs denoted by [[CG]] in SEQ ID NO: 5055;
110. CpGs whose cancer index value is determined are located within at least DMR 55 defined by SEQ ID NO: 5055, and wherein when the cancer index for the individual is less than about 0.05 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least two CpG from DMR 55, and more preferably wherein the cancer index value is the mean β-value for the CpGs denoted by [[CG]] in SEQ ID NO: 5055;
111. CpGs whose cancer index value is determined are located within at least DMR 56 defined by SEQ ID NO: 5056, and wherein when the cancer index for the individual is about 0.016 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 56, and more preferably wherein the cancer index value is the mean β-value for the CpGs denoted by [[CG]] in SEQ ID NO: 5056;
112. CpGs whose cancer index value is determined are located within at least DMR 56 defined by SEQ ID NO: 5056, and wherein when the cancer index for the individual is less than about 0.016 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 56, and more preferably wherein the cancer index value is the mean β-value for the CpGs denoted by [[CG]] in SEQ ID NO: 5056;
113. CpGs whose cancer index value is determined are located within at least DMR 57 defined by SEQ ID NO: 5057, and wherein when the cancer index for the individual is about 0.036 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least three CpG from DMR 57, and more preferably wherein the cancer index value is the mean β-value for the CpGs denoted by [[CG]] in SEQ ID NO: 5057;
114. CpGs whose cancer index value is determined are located within at least DMR 57 defined by SEQ ID NO: 5057, and wherein when the cancer index for the individual is less than about 0.036 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least three CpG from DMR 57, and more preferably wherein the cancer index value is the mean β-value for the CpGs denoted by [[CG]] in SEQ ID NO: 5057;
115. CpGs whose cancer index value is determined are located within at least DMR 58 defined by SEQ ID NO: 5058, and wherein when the cancer index for the individual is about 0.085 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least one CpG from DMR 58, and more preferably wherein the cancer index value is the mean β-value for the CpGs denoted by [[CG]] in SEQ ID NO: 5058;
116. CpGs whose cancer index value is determined are located within at least DMR 58 defined by SEQ ID NO: 5058, and wherein when the cancer index for the individual is less than about 0.085 or more the individual is classified as not having cancer or as having a high risk of cancer development, and wherein the sensitivity of the assay is at least 77.00% and the specificity of the assay is at least 75.00%, preferably wherein the panel of one or more CpGs comprises at least one CpG from DMR 58, and more preferably wherein the cancer index value is the mean β-value for the CpGs denoted by [[CG]] in SEQ ID NO: 5058;
117. CpGs whose cancer index value is determined are located within at least DMR 59 defined by SEQ ID NO: 5059, and wherein when the cancer index for the individual is about 0.028 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least three CpG from DMR 59, and more preferably wherein the cancer index value is the mean β-value for the CpGs denoted by [[CG]] in SEQ ID NO: 5059;
118. CpGs whose cancer index value is determined are located within at least DMR 59 defined by SEQ ID NO: 5059, and wherein when the cancer index for the individual is less than about 0.028 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least three CpG from DMR 59, and more preferably wherein the cancer index value is the mean β-value for the CpGs denoted by [[CG]] in SEQ ID NO: 5059;
119. CpGs whose cancer index value is determined are located within at least DMR 60 defined by SEQ ID NO: 5060, and wherein when the cancer index for the individual is about 0.042 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least three CpG from DMR 60, and more preferably wherein the cancer index value is the mean β-value for the CpGs denoted by [[CG]] in SEQ ID NO: 5060;
120. CpGs whose cancer index value is determined are located within at least DMR 60 defined by SEQ ID NO: 5060, and wherein when the cancer index for the individual is less than about 0.042 the individual is classified as not having CIN3 and/or cancer or as having a low risk of CIN3 and/or cancer development, and wherein the sensitivity of the assay is at least 80.00% and the specificity of the assay is at least 75.00%, preferably wherein the panel of one or more CpGs comprises at least three CpG from DMR 60, and more preferably wherein the cancer index value is the mean β-value for the CpGs denoted by [[CG]] in SEQ ID NO: 5060;
121. CpGs whose cancer index value is determined are located within at least DMR 61 defined by SEQ ID NO: 5061, and wherein when the cancer index for the individual is about 0.037 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 61, and more preferably wherein the cancer index value is the mean β-value for the CpGs denoted by [[CG]] in SEQ ID NO: 5061;
122. CpGs whose cancer index value is determined are located within at least DMR 61 defined by SEQ ID NO: 5061, and wherein when the cancer index for the individual is less than about 0.037 the individual is classified as not having CIN3 and/or cancer or as having a low risk of CIN3 and/or cancer development, and wherein the sensitivity of the assay is at least 77.00% and the specificity of the assay is at least 75.00%, DMR 61, and more preferably wherein the cancer index value is the mean β-value for the CpGs denoted by [[CG]] in SEQ ID NO: 5061;
123. CpGs whose cancer index value is determined are located within at least DMR 62 defined by SEQ ID NO: 5062, and wherein when the cancer index for the individual is about 0.057 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least two CpG from DMR 62, and more preferably wherein the cancer index value is the mean β-value for the CpGs denoted by [[CG]] in SEQ ID NO: 5062;
124. CpGs whose cancer index value is determined are located within at least DMR 62 defined by SEQ ID NO: 5062, and wherein when the cancer index for the individual is less than about 0.057 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least two CpG from DMR 62, and more preferably wherein the cancer index value is the mean β-value for the CpGs denoted by [[CG]] in SEQ ID NO: 5062;
125. CpGs whose cancer index value is determined are located within at least DMR 63 defined by SEQ ID NO: 5063, and wherein when the cancer index for the individual is about 0.031 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eleven CpG from DMR 63, and more preferably wherein the cancer index value is the mean β-value for the CpGs denoted by [[CG]] in SEQ ID NO: 5063;
126. CpGs whose cancer index value is determined are located within at least DMR 63 defined by SEQ ID NO: 5063, and wherein when the cancer index for the individual is less than about 0.031 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eleven CpG from DMR 63, and more preferably wherein the cancer index value is the mean β-value for the CpGs denoted by [[CG]] in SEQ ID NO: 5063;
127. CpGs whose cancer index value is determined are located within at least DMR 64 defined by SEQ ID NO: 5064, and wherein when the cancer index for the individual is about 0.031 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eleven CpG from DMR 64, and more preferably wherein the cancer index value is the mean β-value for the CpGs denoted by [[CG]] in SEQ ID NO: 5064;
128. CpGs whose cancer index value is determined are located within at least DMR 64 defined by SEQ ID NO: 5064, and wherein when the cancer index for the individual is less than about 0.031 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eleven CpG from DMR 64, and more preferably wherein the cancer index value is the mean β-value for the CpGs denoted by [[CG]] in SEQ ID NO: 5064;
129. CpGs whose cancer index value is determined are located within at least DMR 65 defined by SEQ ID NO: 5065, and wherein when the cancer index for the individual is about 0.031 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eleven CpG from DMR 65, and more preferably wherein the cancer index value is the mean β-value for the CpGs denoted by [[CG]] in SEQ ID NO: 5065;
130. CpGs whose cancer index value is determined are located within at least DMR 65 defined by SEQ ID NO: 5065, and wherein when the cancer index for the individual is less than about 0.031 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eleven CpG from DMR 65, and more preferably wherein the cancer index value is the mean β-value for the CpGs denoted by [[CG]] in SEQ ID NO: 5065;
131. CpGs whose cancer index value is determined are located within at least DMR 66 defined by SEQ ID NO: 5066, and wherein when the cancer index for the individual is about 0.031 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eleven CpG from DMR 66, and more preferably wherein the cancer index value is the mean β-value for the CpGs denoted by [[CG]] in SEQ ID NO: 5066;
132. CpGs whose cancer index value is determined are located within at least DMR 66 defined by SEQ ID NO: 5066, and wherein when the cancer index for the individual is less than about 0.031 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eleven CpG from DMR 66, and more preferably wherein the cancer index value is the mean β-value for the CpGs denoted by [[CG]] in SEQ ID NO: 5066;
133. CpGs whose cancer index value is determined are located within at least DMR 67 defined by SEQ ID NO: 5067, and wherein when the cancer index for the individual is about 0.041 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least five CpG from DMR 67, and more preferably wherein the cancer index value is the mean β-value for the CpGs denoted by [[CG]] in SEQ ID NO: 5067;
134. CpGs whose cancer index value is determined are located within at least DMR 67 defined by SEQ ID NO: 5067, and wherein when the cancer index for the individual is less than about 0.041 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least five CpG from DMR 67, and more preferably wherein the cancer index value is the mean β-value for the CpGs denoted by [[CG]] in SEQ ID NO: 5067;
135. CpGs whose cancer index value is determined are located within at least DMR 68 defined by SEQ ID NO: 5068, and wherein when the cancer index for the individual is about 0.037 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least four CpG from DMR 68, and more preferably wherein the cancer index value is the mean β-value for the CpGs denoted by [[CG]] in SEQ ID NO: 5068;
136. CpGs whose cancer index value is determined are located within at least DMR 68 defined by SEQ ID NO: 5068, and wherein when the cancer index for the individual is less than about 0.037 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least four CpG from DMR 68, and more preferably wherein the cancer index value is the mean β-value for the CpGs denoted by [[CG]] in SEQ ID NO: 5068;
137. CpGs whose cancer index value is determined are located within at least DMR 69 defined by SEQ ID NO: 5069, and wherein when the cancer index for the individual is about 0.05 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least three CpG from DMR 69, and more preferably wherein the cancer index value is the mean β-value for the CpGs denoted by [[CG]] in SEQ ID NO: 5069;
138. CpGs whose cancer index value is determined are located within at least DMR 69 defined by SEQ ID NO: 5069, and wherein when the cancer index for the individual is less than about 0.05 the individual is classified as not having CIN3 and/or cancer or as having a low risk of CIN3 and/or cancer development, and wherein the sensitivity of the assay is at least 82.00% and the specificity of the assay is at least 75.00%, preferably wherein the panel of one or more CpGs comprises at least three CpG from DMR 69, and more preferably wherein the cancer index value is the mean β-value for the CpGs denoted by [[CG]] in SEQ ID NO: 5069;
139. CpGs whose cancer index value is determined are located within at least DMR 70 defined by SEQ ID NO: 5070, and wherein when the cancer index for the individual is about 0.049 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 70, and more preferably wherein the cancer index value is the mean β-value for the CpGs denoted by [[CG]] in SEQ ID NO: 5070;
140. CpGs whose cancer index value is determined are located within at least DMR 70 defined by SEQ ID NO: 5070, and wherein when the cancer index for the individual is less than about 0.049 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 70, and more preferably wherein the cancer index value is the mean β-value for the CpGs denoted by [[CG]] in SEQ ID NO: 5070;
141. CpGs whose cancer index value is determined are located within at least DMR 71 defined by SEQ ID NO: 5071, and wherein when the cancer index for the individual is about 0.041 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 71, and more preferably wherein the cancer index value is the mean β-value for the CpGs denoted by [[CG]] in SEQ ID NO: 5071;
142. CpGs whose cancer index value is determined are located within at least DMR 71 defined by SEQ ID NO: 5071, and wherein when the cancer index for the individual is less than about 0.041 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 71, and more preferably wherein the cancer index value is the mean β-value for the CpGs denoted by [[CG]] in SEQ ID NO: 5071;
143. CpGs whose cancer index value is determined are located within at least DMR 72 defined by SEQ ID NO: 5072, and wherein when the cancer index for the individual is about 0.06 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 72, and more preferably wherein the cancer index value is the mean β-value for the CpGs denoted by [[CG]] in SEQ ID NO: 5072;
144. CpGs whose cancer index value is determined are located within at least DMR 72 defined by SEQ ID NO: 5072, and wherein when the cancer index for the individual is less than about 0.06 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 72, and more preferably wherein the cancer index value is the mean β-value for the CpGs denoted by [[CG]] in SEQ ID NO: 5072;
145. CpGs whose cancer index value is determined are located within at least DMR 73 defined by SEQ ID NO: 5073, and wherein when the cancer index for the individual is about 0.042 or more the individual is classified as having CIN3 and/or cancer or as having a high risk of CIN3 and/or cancer development, and wherein the sensitivity of the assay is at least 79.00% and the specificity of the assay is at least 75.00%, DMR 73, and more preferably wherein the cancer index value is the mean β-value for the CpGs denoted by [[CG]] in SEQ ID NO: 5073;
146. CpGs whose cancer index value is determined are located within at least DMR 73 defined by SEQ ID NO: 5073, and wherein when the cancer index for the individual is less than about 0.042 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least four CpG from DMR 73, and more preferably wherein the cancer index value is the mean β-value for the CpGs denoted by [[CG]] in SEQ ID NO: 5073;
147. CpGs whose cancer index value is determined are located within at least DMR 74 defined by SEQ ID NO: 5074, and wherein when the cancer index for the individual is about 0.042 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least four CpG from DMR 74, and more preferably wherein the cancer index value is the mean β-value for the CpGs denoted by [[CG]] in SEQ ID NO: 5074;
148. CpGs whose cancer index value is determined are located within at least DMR 74 defined by SEQ ID NO: 5074, and wherein when the cancer index for the individual is less than about 0.042 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least four CpG from DMR 74, and more preferably wherein the cancer index value is the mean β-value for the CpGs denoted by [[CG]] in SEQ ID NO: 5074;
149. CpGs whose cancer index value is determined are located within at least DMR 75 defined by SEQ ID NO: 5075, and wherein when the cancer index for the individual is about 0.042 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least four CpG from DMR 75, and more preferably wherein the cancer index value is the mean β-value for the CpGs denoted by [[CG]] in SEQ ID NO: 5075;
150. CpGs whose cancer index value is determined are located within at least DMR 75 defined by SEQ ID NO: 5075, and wherein when the cancer index for the individual is less than about 0.042 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least four CpG from DMR 75, and more preferably wherein the cancer index value is the mean β-value for the CpGs denoted by [[CG]] in SEQ ID NO: 5075;
151. CpGs whose cancer index value is determined are located within at least DMR 76 defined by SEQ ID NO: 5076, and wherein when the cancer index for the individual is about 0.042 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least four CpG from DMR 76, and more preferably wherein the cancer index value is the mean β-value for the CpGs denoted by [[CG]] in SEQ ID NO: 5076;
152. CpGs whose cancer index value is determined are located within at least DMR 76 defined by SEQ ID NO: 5076, and wherein when the cancer index for the individual is less than about 0.042 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least four CpG from DMR 76, and more preferably wherein the cancer index value is the mean β-value for the CpGs denoted by [[CG]] in SEQ ID NO: 5076;
153. CpGs whose cancer index value is determined are located within at least DMR 77 defined by SEQ ID NO: 5077, and wherein when the cancer index for the individual is about 0.038 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least five CpG from DMR 77, and more preferably wherein the cancer index value is the mean β-value for the CpGs denoted by [[CG]] in SEQ ID NO: 5077;
154. CpGs whose cancer index value is determined are located within at least DMR 77 defined by SEQ ID NO: 5077, and wherein when the cancer index for the individual is less than about 0.038 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least five CpG from DMR 77, and more preferably wherein the cancer index value is the mean β-value for the CpGs denoted by [[CG]] in SEQ ID NO: 5077;
155. CpGs whose cancer index value is determined are located within at least DMR 78 defined by SEQ ID NO: 5078, and wherein when the cancer index for the individual is about 0.016 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least one CpG from DMR 78, and more preferably wherein the cancer index value is the mean β-value for the CpGs denoted by [[CG]] in SEQ ID NO: 5078;
156. CpGs whose cancer index value is determined are located within at least DMR 78 defined by SEQ ID NO: 5078, and wherein when the cancer index for the individual is less than about 0.016 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least one CpG from DMR 78, and more preferably wherein the cancer index value is the mean β-value for the CpGs denoted by [[CG]] in SEQ ID NO: 5078;
157. CpGs whose cancer index value is determined are located within at least DMR 79 defined by SEQ ID NO: 5079, and wherein when the cancer index for the individual is about 0.033 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least eight CpG from DMR 79, and more preferably wherein the cancer index value is the mean β-value for the CpGs denoted by [[CG]] in SEQ ID NO: 5079;
158. CpGs whose cancer index value is determined are located within at least DMR 79 defined by SEQ ID NO: 5079, and wherein when the cancer index for the individual is less than about 0.033 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least eight CpG from DMR 79, and more preferably wherein the cancer index value is the mean β-value for the CpGs denoted by [[CG]] in SEQ ID NO: 5079;
159. CpGs whose cancer index value is determined are located within at least DMR 80 defined by SEQ ID NO: 5080, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least ten CpG from DMR 80, and more preferably wherein the cancer index value is the mean β-value for the CpGs denoted by [[CG]] in SEQ ID NO: 5080;
160. CpGs whose cancer index value is determined are located within at least DMR 80 defined by SEQ ID NO: 5080, and wherein when the cancer index for the individual is less than about 0.032 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least ten CpG from DMR 80, and more preferably wherein the cancer index value is the mean β-value for the CpGs denoted by [[CG]] in SEQ ID NO: 5080;
161. CpGs whose cancer index value is determined are located within at least DMR 81 defined by SEQ ID NO: 5081, and wherein when the cancer index for the individual is about 0.037 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least four CpG from DMR 81, and more preferably wherein the cancer index value is the mean β-value for the CpGs denoted by [[CG]] in SEQ ID NO: 5081;
162. CpGs whose cancer index value is determined are located within at least DMR 81 defined by SEQ ID NO: 5081, and wherein when the cancer index for the individual is less than about 0.037 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least four CpG from DMR 81, and more preferably wherein the cancer index value is the mean β-value for the CpGs denoted by [[CG]] in SEQ ID NO: 5081;
163. CpGs whose cancer index value is determined are located within at least DMR 82 defined by SEQ ID NO: 5082, and wherein when the cancer index for the individual is about 0.033 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least ten CpG from DMR 82, and more preferably wherein the cancer index value is the mean β-value for the CpGs denoted by [[CG]] in SEQ ID NO: 5082;
164. CpGs whose cancer index value is determined are located within at least DMR 82 defined by SEQ ID NO: 5082, and wherein when the cancer index for the individual is less than about 0.033 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least ten CpG from DMR 82, and more preferably wherein the cancer index value is the mean β-value for the CpGs denoted by [[CG]] in SEQ ID NO: 5082;
165. CpGs whose cancer index value is determined are located within at least DMR 83 defined by SEQ ID NO: 5083, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 83, and more preferably wherein the cancer index value is the mean β-value for the CpGs denoted by [[CG]] in SEQ ID NO: 5083;
166. CpGs whose cancer index value is determined are located within at least DMR 83 defined by SEQ ID NO: 5083, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 83, and more preferably wherein the cancer index value is the mean β-value for the CpGs denoted by [[CG]] in SEQ ID NO: 5083;
167. CpGs whose cancer index value is determined are located within at least DMR 84 defined by SEQ ID NO: 5084, and wherein when the cancer index for the individual is about 0.033 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least ten CpG from DMR 84, and more preferably wherein the cancer index value is the mean β-value for the CpGs denoted by [[CG]] in SEQ ID NO: 5084;
168. CpGs whose cancer index value is determined are located within at least DMR 84 defined by SEQ ID NO: 5084, and wherein when the cancer index for the individual is less than about 0.033 the individual is classified as not having CIN3 and/or cancer or as having a low risk of CIN3 and/or cancer development, and wherein the sensitivity of the assay is at least 75.00% and the specificity of the assay is at least 75.00%, DMR 84, and more preferably wherein the cancer index value is the mean β-value for the CpGs denoted by [[CG]] in SEQ ID NO: 5084;
169. CpGs whose cancer index value is determined are located within at least DMR 85 defined by SEQ ID NO: 5085, and wherein when the cancer index for the individual is about 0.033 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least ten CpG from DMR 85, and more preferably wherein the cancer index value is the mean β-value for the CpGs denoted by [[CG]] in SEQ ID NO: 5085;
170. CpGs whose cancer index value is determined are located within at least DMR 85 defined by SEQ ID NO: 5085, and wherein when the cancer index for the individual is less than about 0.033 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least ten CpG from DMR 85, and more preferably wherein the cancer index value is the mean β-value for the CpGs denoted by [[CG]] in SEQ ID NO: 5085;
171. CpGs whose cancer index value is determined are located within at least DMR 86 defined by SEQ ID NO: 5086, and wherein when the cancer index for the individual is about 0.033 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least ten CpG from DMR 86, and more preferably wherein the cancer index value is the mean β-value for the CpGs denoted by [[CG]] in SEQ ID NO: 5086;
172. CpGs whose cancer index value is determined are located within at least DMR 86 defined by SEQ ID NO: 5086, and wherein when the cancer index for the individual is less than about 0.033 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least ten CpG from DMR 86, and more preferably wherein the cancer index value is the mean β-value for the CpGs denoted by [[CG]] in SEQ ID NO: 5086;
173. CpGs whose cancer index value is determined are located within at least DMR 87 defined by SEQ ID NO: 5087, and wherein when the cancer index for the individual is about 0.058 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least four CpG from DMR 87, and more preferably wherein the cancer index value is the mean β-value for the CpGs denoted by [[CG]] in SEQ ID NO: 5087;
174. CpGs whose cancer index value is determined are located within at least DMR 87 defined by SEQ ID NO: 5087, and wherein when the cancer index for the individual is less than about 0.058 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least four CpG from DMR 87, and more preferably wherein the cancer index value is the mean β-value for the CpGs denoted by [[CG]] in SEQ ID NO: 5087;
175. CpGs whose cancer index value is determined are located within at least DMR 88 defined by SEQ ID NO: 5088, and wherein when the cancer index for the individual is about 0.042 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least twelve CpG from DMR 88, and more preferably wherein the cancer index value is the mean β-value for the CpGs denoted by [[CG]] in SEQ ID NO: 5088;
176. CpGs whose cancer index value is determined are located within at least DMR 88 defined by SEQ ID NO: 5088, and wherein when the cancer index for the individual is less than about 0.042 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least twelve CpG from DMR 88, and more preferably wherein the cancer index value is the mean β-value for the CpGs denoted by [[CG]] in SEQ ID NO: 5088;
177. CpGs whose cancer index value is determined are located within at least DMR 89 defined by SEQ ID NO: 5089, and wherein when the cancer index for the individual is about 0.036 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least five CpG from DMR 89, and more preferably wherein the cancer index value is the mean β-value for the CpGs denoted by [[CG]] in SEQ ID NO: 5089;
178. CpGs whose cancer index value is determined are located within at least DMR 89 defined by SEQ ID NO: 5089, and wherein when the cancer index for the individual is less than about 0.036 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least five CpG from DMR 89, and more preferably wherein the cancer index value is the mean β-value for the CpGs denoted by [[CG]] in SEQ ID NO: 5089;
179. CpGs whose cancer index value is determined are located within at least DMR 90 defined by SEQ ID NO: 5090, and wherein when the cancer index for the individual is about 0.04 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least eight CpG from DMR 90, and more preferably wherein the cancer index value is the mean β-value for the CpGs denoted by [[CG]] in SEQ ID NO: 5090;
180. CpGs whose cancer index value is determined are located within at least DMR 90 defined by SEQ ID NO: 5090, and wherein when the cancer index for the individual is less than about 0.04 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least eight CpG from DMR 90, and more preferably wherein the cancer index value is the mean β-value for the CpGs denoted by [[CG]] in SEQ ID NO: 5090;
181. CpGs whose cancer index value is determined are located within at least DMR 91 defined by SEQ ID NO: 5091, and wherein when the cancer index for the individual is about 0.04 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least eight CpG from DMR 91, and more preferably wherein the cancer index value is the mean β-value for the CpGs denoted by [[CG]] in SEQ ID NO: 5091;
182. CpGs whose cancer index value is determined are located within at least DMR 91 defined by SEQ ID NO: 5091, and wherein when the cancer index for the individual is less than about 0.04 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least eight CpG from DMR 91, and more preferably wherein the cancer index value is the mean β-value for the CpGs denoted by [[CG]] in SEQ ID NO: 5091;
183. CpGs whose cancer index value is determined are located within at least DMR 92 defined by SEQ ID NO: 5092, and wherein when the cancer index for the individual is about 0.042 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least seven CpG from DMR 92, and more preferably wherein the cancer index value is the mean β-value for the CpGs denoted by [[CG]] in SEQ ID NO: 5092;
184. CpGs whose cancer index value is determined are located within at least DMR 92 defined by SEQ ID NO: 5092, and wherein when the cancer index for the individual is less than about 0.042 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least seven CpG from DMR 92, and more preferably wherein the cancer index value is the mean β-value for the CpGs denoted by [[CG]] in SEQ ID NO: 5092;
185. CpGs whose cancer index value is determined are located within at least DMR 93 defined by SEQ ID NO: 5093, and wherein when the cancer index for the individual is about 0.05 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least six CpG from DMR 93, and more preferably wherein the cancer index value is the mean β-value for the CpGs denoted by [[CG]] in SEQ ID NO: 5093;
186. CpGs whose cancer index value is determined are located within at least DMR 93 defined by SEQ ID NO: 5093, and wherein when the cancer index for the individual is less than about 0.05 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least six CpG from DMR 93, and more preferably wherein the cancer index value is the mean β-value for the CpGs denoted by [[CG]] in SEQ ID NO: 5093;
187. CpGs whose cancer index value is determined are located within at least DMR 94 defined by SEQ ID NO: 5094, and wherein when the cancer index for the individual is about 0.05 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least six CpG from DMR 94, and more preferably wherein the cancer index value is the mean β-value for the CpGs denoted by [[CG]] in SEQ ID NO: 5094;
188. CpGs whose cancer index value is determined are located within at least DMR 94 defined by SEQ ID NO: 5094, and wherein when the cancer index for the individual is less than about 0.05 the individual is classified as not having CIN3 and/or cancer or as having a low risk of CIN3 and/or cancer development, and wherein the sensitivity of the assay is at least 79.00% and the specificity of the assay is at least 75.00%, preferably wherein the panel of one or more CpGs comprises at least six CpG from DMR 94, and more preferably wherein the cancer index value is the mean β-value for the CpGs denoted by [[CG]] in SEQ ID NO: 5094;
189. CpGs whose cancer index value is determined are located within at least DMR 95 defined by SEQ ID NO: 5095, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 95, and more preferably wherein the cancer index value is the mean β-value for the CpGs denoted by [[CG]] in SEQ ID NO: 5095;
190. CpGs whose cancer index value is determined are located within at least DMR 95 defined by SEQ ID NO: 5095, and wherein when the cancer index for the individual is less than about 0.032 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 95, and more preferably wherein the cancer index value is the mean β-value for the CpGs denoted by [[CG]] in SEQ ID NO: 5095;
191. CpGs whose cancer index value is determined are located within at least DMR 96 defined by SEQ ID NO: 5096, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, DMR 96, and more preferably wherein the cancer index value is the mean β-value for the CpGs denoted by [[CG]] in SEQ ID NO: 5096;
192. CpGs whose cancer index value is determined are located within at least DMR 96 defined by SEQ ID NO: 5096, and wherein when the cancer index for the individual is less than about 0.032 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 96, and more preferably wherein the cancer index value is the mean β-value for the CpGs denoted by [[CG]] in SEQ ID NO: 5096;
193. CpGs whose cancer index value is determined are located within at least DMR 97 defined by SEQ ID NO: 5097, and wherein when the cancer index for the individual is about 0.053 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least seven CpG from DMR 97, and more preferably wherein the cancer index value is the mean β-value for the CpGs denoted by [[CG]] in SEQ ID NO: 5097;
194. CpGs whose cancer index value is determined are located within at least DMR 97 defined by SEQ ID NO: 5097, and wherein when the cancer index for the individual is less than about 0.053 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least seven CpG from DMR 97, and more preferably wherein the cancer index value is the mean β-value for the CpGs denoted by [[CG]] in SEQ ID NO: 5097;
195. CpGs whose cancer index value is determined are located within at least DMR 98 defined by SEQ ID NO: 5098, and wherein when the cancer index for the individual is about 0.024 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least four CpG from DMR 98, and more preferably wherein the cancer index value is the mean β-value for the CpGs denoted by [[CG]] in SEQ ID NO: 5098;
196. CpGs whose cancer index value is determined are located within at least DMR 98 defined by SEQ ID NO: 5098, and wherein when the cancer index for the individual is less than about 0.024 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least four CpG from DMR 98, and more preferably wherein the cancer index value is the mean β-value for the CpGs denoted by [[CG]] in SEQ ID NO: 5098;
197. CpGs whose cancer index value is determined are located within at least DMR 99 defined by SEQ ID NO: 5099, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 99, and more preferably wherein the cancer index value is the mean β-value for the CpGs denoted by [[CG]] in SEQ ID NO: 5099;
198. CpGs whose cancer index value is determined are located within at least DMR 99 defined by SEQ ID NO: 5099, and wherein when the cancer index for the individual is less than about 0.032 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 99, and more preferably wherein the cancer index value is the mean β-value for the CpGs denoted by [[CG]] in SEQ ID NO: 5099;
199. CpGs whose cancer index value is determined are located within at least DMR 100 defined by SEQ ID NO: 5100, and wherein when the cancer index for the individual is about 0.036 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least seven CpG from DMR 100, and more preferably wherein the cancer index value is the mean β-value for the CpGs denoted by [[CG]] in SEQ ID NO: 5100;
200. CpGs whose cancer index value is determined are located within at least DMR 100 defined by SEQ ID NO: 5100, and wherein when the cancer index for the individual is less than about 0.036 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least seven CpG from DMR 100, and more preferably wherein the cancer index value is the mean β-value for the CpGs denoted by [[CG]] in SEQ ID NO: 5100;
201. CpGs whose cancer index value is determined are located within at least DMR 101 defined by SEQ ID NO: 5101, and wherein when the cancer index for the individual is about 0.034 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least nine CpG from DMR 101, and more preferably wherein the cancer index value is the mean β-value for the CpGs denoted by [[CG]] in SEQ ID NO: 5101;
202. CpGs whose cancer index value is determined are located within at least DMR 101 defined by SEQ ID NO: 5101, and wherein when the cancer index for the individual is less than about 0.034 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least nine CpG from DMR 101, and more preferably wherein the cancer index value is the mean β-value for the CpGs denoted by [[CG]] in SEQ ID NO: 5101;
203. CpGs whose cancer index value is determined are located within at least DMR 102 defined by SEQ ID NO: 5102, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 102, and more preferably wherein the cancer index value is the mean β-value for the CpGs denoted by [[CG]] in SEQ ID NO: 5102;
204. CpGs whose cancer index value is determined are located within at least DMR 102 defined by SEQ ID NO: 5102, and wherein when the cancer index for the individual is less than about 0.032 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 102, and more preferably wherein the cancer index value is the mean β-value for the CpGs denoted by [[CG]] in SEQ ID NO: 5102;
205. CpGs whose cancer index value is determined are located within at least DMR 103 defined by SEQ ID NO: 5103, and wherein when the cancer index for the individual is about 0.034 or more the individual is classified as having CIN3 and/or cancer or as having a high risk of CIN3 and/or cancer development, and wherein the sensitivity of preferably wherein the panel of one or more CpGs comprises at least nine CpG from DMR 103, and more preferably wherein the cancer index value is the mean β-value for the CpGs denoted by [[CG]] in SEQ ID NO: 5103;
206. CpGs whose cancer index value is determined are located within at least DMR 103 defined by SEQ ID NO: 5103, and wherein when the cancer index for the individual is less than about 0.034 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least nine CpG from DMR 103, and more preferably wherein the cancer index value is the mean β-value for the CpGs denoted by [[CG]] in SEQ ID NO: 5103;
207. CpGs whose cancer index value is determined are located within at least DMR 104 defined by SEQ ID NO: 5104, and wherein when the cancer index for the individual is about 0.024 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least four CpG from DMR 104, and more preferably wherein the cancer index value is the mean β-value for the CpGs denoted by [[CG]] in SEQ ID NO: 5104;
208. CpGs whose cancer index value is determined are located within at least DMR 104 defined by SEQ ID NO: 5104, and wherein when the cancer index for the individual is less than about 0.024 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least four CpG from DMR 104, and more preferably wherein the cancer index value is the mean β-value for the CpGs denoted by [[CG]] in SEQ ID NO: 5104;
209. CpGs whose cancer index value is determined are located within at least DMR 105 defined by SEQ ID NO: 5105, and wherein when the cancer index for the individual is about 0.024 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least four CpG from DMR 105, and more preferably wherein the cancer index value is the mean β-value for the CpGs denoted by [[CG]] in SEQ ID NO: 5105;
210. CpGs whose cancer index value is determined are located within at least DMR 105 defined by SEQ ID NO: 5105, and wherein when the cancer index for the individual is less than about 0.024 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least four CpG from DMR 105, and more preferably wherein the cancer index value is the mean β-value for the CpGs denoted by [[CG]] in SEQ ID NO: 5105;
211. CpGs whose cancer index value is determined are located within at least DMR 106 defined by SEQ ID NO: 5106, and wherein when the cancer index for the individual is about 0.036 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least seven CpG from DMR 106, and more preferably wherein the cancer index value is the mean β-value for the CpGs denoted by [[CG]] in SEQ ID NO: 5106;
212. CpGs whose cancer index value is determined are located within at least DMR 106 defined by SEQ ID NO: 5106, and wherein when the cancer index for the individual is less than about 0.036 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least seven CpG from DMR 106, and more preferably wherein the cancer index value is the mean β-value for the CpGs denoted by [[CG]] in SEQ ID NO: 5106;
213. CpGs whose cancer index value is determined are located within at least DMR 107 defined by SEQ ID NO: 5107, and wherein when the cancer index for the individual is about 0.036 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least seven CpG from DMR 107, and more preferably wherein the cancer index value is the mean β-value for the CpGs denoted by [[CG]] in SEQ ID NO: 5107;
214. CpGs whose cancer index value is determined are located within at least DMR 107 defined by SEQ ID NO: 5107, and wherein when the cancer index for the individual is less than about 0.036 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least seven CpG from DMR 107, and more preferably wherein the cancer index value is the mean β-value for the CpGs denoted by [[CG]] in SEQ ID NO: 5107;
215. CpGs whose cancer index value is determined are located within at least DMR 108 defined by SEQ ID NO: 5108, and wherein when the cancer index for the individual is about 0.051 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 108, and more preferably wherein the cancer index value is the mean β-value for the CpGs denoted by [[CG]] in SEQ ID NO: 5108;
216. CpGs whose cancer index value is determined are located within at least DMR 108 defined by SEQ ID NO: 5108, and wherein when the cancer index for the individual is less than about 0.051 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 108, and more preferably wherein the cancer index value is the mean β-value for the CpGs denoted by [[CG]] in SEQ ID NO: 5108;
217. CpGs whose cancer index value is determined are located within at least DMR 109 defined by SEQ ID NO: 5109, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 109, and more preferably wherein the cancer index value is the mean β-value for the CpGs denoted by [[CG]] in SEQ ID NO: 5109;
218. CpGs whose cancer index value is determined are located within at least DMR 109 defined by SEQ ID NO: 5109, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least four CpG from DMR 109, and more preferably wherein the cancer index value is the mean β-value for the CpGs denoted by [[CG]] in SEQ ID NO: 5109;
219. CpGs whose cancer index value is determined are located within at least DMR 110 defined by SEQ ID NO: 5110, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 110, and more preferably wherein the cancer index value is the mean β-value for the CpGs denoted by [[CG]] in SEQ ID NO: 5110;
220. CpGs whose cancer index value is determined are located within at least DMR 110 defined by SEQ ID NO: 5110, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least four CpG from DMR 110, and more preferably wherein the cancer index value is the mean β-value for the CpGs denoted by [[CG]] in SEQ ID NO: 5110;
221. CpGs whose cancer index value is determined are located within at least DMR 111 defined by SEQ ID NO: 5111, and wherein when the cancer index for the individual is about 0.057 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least five CpG from DMR 111, and more preferably wherein the cancer index value is the mean β-value for the CpGs denoted by [[CG]] in SEQ ID NO: 5111;
222. CpGs whose cancer index value is determined are located within at least DMR 111 defined by SEQ ID NO: 5111, and wherein when the cancer index for the individual is less than about 0.057 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least five CpG from DMR 111, and more preferably wherein the cancer index value is the mean β-value for the CpGs denoted by [[CG]] in SEQ ID NO: 5111;
223. CpGs whose cancer index value is determined are located within at least DMR 112 defined by SEQ ID NO: 5112, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 112, and more preferably wherein the cancer index value is the mean β-value for the CpGs denoted by [[CG]] in SEQ ID NO: 5112;
224. CpGs whose cancer index value is determined are located within at least DMR 112 defined by SEQ ID NO: 5112, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least four CpG from DMR 112, and more preferably wherein the cancer index value is the mean β-value for the CpGs denoted by [[CG]] in SEQ ID NO: 5112;
225. CpGs whose cancer index value is determined are located within at least DMR 113 defined by SEQ ID NO: 5113, and wherein when the cancer index for the individual is about 0.057 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least five CpG from DMR 113, and more preferably wherein the cancer index value is the mean β-value for the CpGs denoted by [[CG]] in SEQ ID NO: 5113;
226. CpGs whose cancer index value is determined are located within at least DMR 113 defined by SEQ ID NO: 5113, and wherein when the cancer index for the individual is less than about 0.057 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least five CpG from DMR 113, and more preferably wherein the cancer index value is the mean β-value for the CpGs denoted by [[CG]] in SEQ ID NO: 5113;
227. CpGs whose cancer index value is determined are located within at least DMR 114 defined by SEQ ID NO: 5114, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 114, and more preferably wherein the cancer index value is the mean β-value for the CpGs denoted by [[CG]] in SEQ ID NO: 5114;
228. CpGs whose cancer index value is determined are located within at least DMR 114 defined by SEQ ID NO: 5114, and wherein when the cancer index for the individual is less than about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 114, and more preferably wherein the cancer index value is the mean β-value for the CpGs denoted by [[CG]] in SEQ ID NO: 5114;
229. CpGs whose cancer index value is determined are located within at least DMR 115 defined by SEQ ID NO: 5115, and wherein when the cancer index for the individual is about 0.037 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least eight CpG from DMR 115, and more preferably wherein the cancer index value is the mean β-value for the CpGs denoted by [[CG]] in SEQ ID NO: 5115;
230. CpGs whose cancer index value is determined are located within at least DMR 115 defined by SEQ ID NO: 5115, and wherein when the cancer index for the individual is less than about 0.037 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least eight CpG from DMR 115, and more preferably wherein the cancer index value is the mean β-value for the CpGs denoted by [[CG]] in SEQ ID NO: 5115;
231. CpGs whose cancer index value is determined are located within at least DMR 116 defined by SEQ ID NO: 5116, and wherein when the cancer index for the individual is about 0.047 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least one CpG from DMR 116, and more preferably wherein the cancer index value is the mean β-value for the CpGs denoted by [[CG]] in SEQ ID NO: 5116;
232. CpGs whose cancer index value is determined are located within at least DMR 116 defined by SEQ ID NO: 5116, and wherein when the cancer index for the individual is less than about 0.047 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least one CpG from DMR 116, and more preferably wherein the cancer index value is the mean β-value for the CpGs denoted by [[CG]] in SEQ ID NO: 5116;
233. CpGs whose cancer index value is determined are located within at least DMR 117 defined by SEQ ID NO: 5117, and wherein when the cancer index for the individual is about 0.035 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least eight CpG from DMR 117, and more preferably wherein the cancer index value is the mean β-value for the CpGs denoted by [[CG]] in SEQ ID NO: 5117;
234. CpGs whose cancer index value is determined are located within at least DMR 117 defined by SEQ ID NO: 5117, and wherein when the cancer index for the individual is less than about 0.035 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least eight CpG from DMR 117, and more preferably wherein the cancer index value is the mean β-value for the CpGs denoted by [[CG]] in SEQ ID NO: 5117;
235. CpGs whose cancer index value is determined are located within at least DMR 118 defined by SEQ ID NO: 5118, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 118, and more preferably wherein the cancer index value is the mean β-value for the CpGs denoted by [[CG]] in SEQ ID NO: 5118;
236. CpGs whose cancer index value is determined are located within at least DMR 118 defined by SEQ ID NO: 5118, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 118, and more preferably wherein the cancer index value is the mean β-value for the CpGs denoted by [[CG]] in SEQ ID NO: 5118;
237. CpGs whose cancer index value is determined are located within at least DMR 119 defined by SEQ ID NO: 5119, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, DMR 119, and more preferably wherein the cancer index value is the mean β-value for the CpGs denoted by [[CG]] in SEQ ID NO: 5119;
238. CpGs whose cancer index value is determined are located within at least DMR 119 defined by SEQ ID NO: 5119, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 119, and more preferably wherein the cancer index value is the mean β-value for the CpGs denoted by [[CG]] in SEQ ID NO: 5119;
239. CpGs whose cancer index value is determined are located within at least DMR 120 defined by SEQ ID NO: 5120, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 120, and more preferably wherein the cancer index value is the mean β-value for the CpGs denoted by [[CG]] in SEQ ID NO: 5120;
240. CpGs whose cancer index value is determined are located within at least DMR 120 defined by SEQ ID NO: 5120, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 120, and more preferably wherein the cancer index value is the mean β-value for the CpGs denoted by [[CG]] in SEQ ID NO: 5120;
241. CpGs whose cancer index value is determined are located within at least DMR 121 defined by SEQ ID NO: 5121, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 121, and more preferably wherein the cancer index value is the mean β-value for the CpGs denoted by [[CG]] in SEQ ID NO: 5121;
242. CpGs whose cancer index value is determined are located within at least DMR 121 defined by SEQ ID NO: 5121, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 121, and more preferably wherein the cancer index value is the mean β-value for the CpGs denoted by [[CG]] in SEQ ID NO: 5121;
243. CpGs whose cancer index value is determined are located within at least DMR 122 defined by SEQ ID NO: 5122, and wherein when the cancer index for the individual is about 0.022 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 122, and more preferably wherein the cancer index value is the mean β-value for the CpGs denoted by [[CG]] in SEQ ID NO: 5122;
244. CpGs whose cancer index value is determined are located within at least DMR 122 defined by SEQ ID NO: 5122, and wherein when the cancer index for the individual is less than about 0.022 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 122, and more preferably wherein the cancer index value is the mean β-value for the CpGs denoted by [[CG]] in SEQ ID NO: 5122;
245. CpGs whose cancer index value is determined are located within at least DMR 123 defined by SEQ ID NO: 5123, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 123, and more preferably wherein the cancer index value is the mean β-value for the CpGs denoted by [[CG]] in SEQ ID NO: 5123;
246. CpGs whose cancer index value is determined are located within at least DMR 123 defined by SEQ ID NO: 5123, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 123, and more preferably wherein the cancer index value is the mean β-value for the CpGs denoted by [[CG]] in SEQ ID NO: 5123;
247. CpGs whose cancer index value is determined are located within at least DMR 124 defined by SEQ ID NO: 5124, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 124, and more preferably wherein the cancer index value is the mean β-value for the CpGs denoted by [[CG]] in SEQ ID NO: 5124;
248. CpGs whose cancer index value is determined are located within at least DMR 124 defined by SEQ ID NO: 5124, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 124, and more preferably wherein the cancer index value is the mean β-value for the CpGs denoted by [[CG]] in SEQ ID NO: 5124;
249. CpGs whose cancer index value is determined are located within at least DMR 125 defined by SEQ ID NO: 5125, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 125, and more preferably wherein the cancer index value is the mean β-value for the CpGs denoted by [[CG]] in SEQ ID NO: 5125;
250. CpGs whose cancer index value is determined are located within at least DMR 125 defined by SEQ ID NO: 5125, and wherein when the cancer index for the individual is less than about 0.043 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least ten CpG from DMR 125, and more preferably wherein the cancer index value is the mean β-value for the CpGs denoted by [[CG]] in SEQ ID NO: 5125;
251. CpGs whose cancer index value is determined are located within at least DMR 126 defined by SEQ ID NO: 5126, and wherein when the cancer index for the individual is about 0.022 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 126, and more preferably wherein the cancer index value is the mean β-value for the CpGs denoted by [[CG]] in SEQ ID NO: 5126;
252. CpGs whose cancer index value is determined are located within at least DMR 126 defined by SEQ ID NO: 5126, and wherein when the cancer index for the individual is less than about 0.022 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 126, and more preferably wherein the cancer index value is the mean β-value for the CpGs denoted by [[CG]] in SEQ ID NO: 5126;
253. CpGs whose cancer index value is determined are located within at least DMR 127 defined by SEQ ID NO: 5127, and wherein when the cancer index for the individual is about 0.043 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least ten CpG from DMR 127, and more preferably wherein the cancer index value is the mean β-value for the CpGs denoted by [[CG]] in SEQ ID NO: 5127;
254. CpGs whose cancer index value is determined are located within at least DMR 127 defined by SEQ ID NO: 5127, and wherein when the cancer index for the individual is less than about 0.043 or more the individual is classified as not having cancer or as having a low risk of cancer development, and wherein the sensitivity of the assay is at least 74.00% and the specificity of the assay is at least 75.00%, preferably wherein the panel of one or more CpGs comprises at least ten CpG from DMR 127, and more preferably wherein the cancer index value is the mean β-value for the CpGs denoted by [[CG]] in SEQ ID NO: 5127;
255. CpGs whose cancer index value is determined are located within at least DMR 128 defined by SEQ ID NO: 5128, and wherein when the cancer index for the individual is about 0.038 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 128, and more preferably wherein the cancer index value is the mean β-value for the CpGs denoted by [[CG]] in SEQ ID NO: 5128;
256. CpGs whose cancer index value is determined are located within at least DMR 128 defined by SEQ ID NO: 5128, and wherein when the cancer index for the individual is less than about 0.038 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least five CpG from DMR 128, and more preferably wherein the cancer index value is the mean β-value for the CpGs denoted by [[CG]] in SEQ ID NO: 5128;
257. CpGs whose cancer index value is determined are located within at least DMR 129 defined by SEQ ID NO: 5129, and wherein when the cancer index for the individual is about 0.038 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 129, and more preferably wherein the cancer index value is the mean β-value for the CpGs denoted by [[CG]] in SEQ ID NO: 5129;
258. CpGs whose cancer index value is determined are located within at least DMR 129 defined by SEQ ID NO: 5129, and wherein when the cancer index for the individual is less than about 0.038 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least five CpG from DMR 129, and more preferably wherein the cancer index value is the mean β-value for the CpGs denoted by [[CG]] in SEQ ID NO: 5129;
259. CpGs whose cancer index value is determined are located within at least DMR 130 defined by SEQ ID NO: 5130, and wherein when the cancer index for the individual is about 0.029 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least four CpG from DMR 130, and more preferably wherein the cancer index value is the mean β-value for the CpGs denoted by [[CG]] in SEQ ID NO: 5130;
260. CpGs whose cancer index value is determined are located within at least DMR 130 defined by SEQ ID NO: 5130, and wherein when the cancer index for the individual is less than about 0.029 the individual is classified as not having CIN3 and/or cancer or as having a low risk of CIN3 and/or cancer development, and wherein the sensitivity of the assay is at least 72.00% and the specificity of the assay is at least 75.00%, DMR 130, and more preferably wherein the cancer index value is the mean β-value for the CpGs denoted by [[CG]] in SEQ ID NO: 5130;
261. CpGs whose cancer index value is determined are located within at least DMR 131 defined by SEQ ID NO: 5131, and wherein when the cancer index for the individual is about 0.038 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 131, and more preferably wherein the cancer index value is the mean β-value for the CpGs denoted by [[CG]] in SEQ ID NO: 5131;
262. CpGs whose cancer index value is determined are located within at least DMR 131 defined by SEQ ID NO: 5131, and wherein when the cancer index for the individual is less than about 0.038 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least five CpG from DMR 131, and more preferably wherein the cancer index value is the mean β-value for the CpGs denoted by [[CG]] in SEQ ID NO: 5131;
263. CpGs whose cancer index value is determined are located within at least DMR 132 defined by SEQ ID NO: 5132, and wherein when the cancer index for the individual is about 0.03 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 132, and more preferably wherein the cancer index value is the mean β-value for the CpGs denoted by [[CG]] in SEQ ID NO: 5132;
264. CpGs whose cancer index value is determined are located within at least DMR 132 defined by SEQ ID NO: 5132, and wherein when the cancer index for the individual is less than about 0.03 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 132, and more preferably wherein the cancer index value is the mean β-value for the CpGs denoted by [[CG]] in SEQ ID NO: 5132;
265. CpGs whose cancer index value is determined are located within at least DMR 133 defined by SEQ ID NO: 5133, and wherein when the cancer index for the individual is about 0.036 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 133, and more preferably wherein the cancer index value is the mean β-value for the CpGs denoted by [[CG]] in SEQ ID NO: 5133;
266. CpGs whose cancer index value is determined are located within at least DMR 133 defined by SEQ ID NO: 5133, and wherein when the cancer index for the individual is less than about 0.036 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 133, and more preferably wherein the cancer index value is the mean β-value for the CpGs denoted by [[CG]] in SEQ ID NO: 5133;
267. CpGs whose cancer index value is determined are located within at least DMR 134 defined by SEQ ID NO: 5134, and wherein when the cancer index for the individual is about 0.051 or more the individual is classified as having CIN3 and/or cancer or as having a high risk of CIN3 and/or cancer development, and wherein the sensitivity of the assay is at least 78.00% and the specificity of the assay is at least 75.00%, preferably wherein the panel of one or more CpGs comprises at least four CpG from DMR 134, and more preferably wherein the cancer index value is the mean β-value for the CpGs denoted by [[CG]] in SEQ ID NO: 5134;
268. CpGs whose cancer index value is determined are located within at least DMR 134 defined by SEQ ID NO: 5134, and wherein when the cancer index for the individual is less than about 0.051 the individual is classified as not having CIN3 and/or cancer or as having a low risk of CIN3 and/or cancer development, and wherein the sensitivity of the assay is at least 78.00% and the specificity of the assay is at least 75.00%, preferably wherein the panel of one or more CpGs comprises at least four CpG from DMR 134, and more preferably wherein the cancer index value is the mean β-value for the CpGs denoted by [[CG]] in SEQ ID NO: 5134;
269. CpGs whose cancer index value is determined are located within at least DMR 135 defined by SEQ ID NO: 5135, and wherein when the cancer index for the individual is about 0.025 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least three CpG from DMR 135, and more preferably wherein the cancer index value is the mean β-value for the CpGs denoted by [[CG]] in SEQ ID NO: 5135;
270. CpGs whose cancer index value is determined are located within at least DMR 135 defined by SEQ ID NO: 5135, and wherein when the cancer index for the individual is less than about 0.025 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least three CpG from DMR 135, and more preferably wherein the cancer index value is the mean β-value for the CpGs denoted by [[CG]] in SEQ ID NO: 5135;
271. CpGs whose cancer index value is determined are located within at least DMR 136 defined by SEQ ID NO: 5136, and wherein when the cancer index for the individual is about 0.036 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 136, and more preferably wherein the cancer index value is the mean β-value for the CpGs denoted by [[CG]] in SEQ ID NO: 5136;
272. CpGs whose cancer index value is determined are located within at least DMR 136 defined by SEQ ID NO: 5136, and wherein when the cancer index for the individual is less than about 0.036 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 136, and more preferably wherein the cancer index value is the mean β-value for the CpGs denoted by [[CG]] in SEQ ID NO: 5136;
273. CpGs whose cancer index value is determined are located within at least DMR 137 defined by SEQ ID NO: 5137, and wherein when the cancer index for the individual is about 0.088 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 137, and more preferably wherein the cancer index value is the mean β-value for the CpGs denoted by [[CG]] in SEQ ID NO: 5137;
274. CpGs whose cancer index value is determined are located within at least DMR 137 defined by SEQ ID NO: 5137, and wherein when the cancer index for the individual is less than about 0.088 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 137, and more preferably wherein the cancer index value is the mean β-value for the CpGs denoted by [[CG]] in SEQ ID NO: 5137;
275. CpGs whose cancer index value is determined are located within at least DMR 138 defined by SEQ ID NO: 5138, and wherein when the cancer index for the individual is about 0.088 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least three CpG from DMR 138, and more preferably wherein the cancer index value is the mean β-value for the CpGs denoted by [[CG]] in SEQ ID NO: 5138;
276. CpGs whose cancer index value is determined are located within at least DMR 138 defined by SEQ ID NO: 5138, and wherein when the cancer index for the individual is less than about 0.088 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least three CpG from DMR 138, and more preferably wherein the cancer index value is the mean β-value for the CpGs denoted by [[CG]] in SEQ ID NO: 5138;
277. CpGs whose cancer index value is determined are located within at least DMR 139 defined by SEQ ID NO: 5139, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 139, and more preferably wherein the cancer index value is the mean β-value for the CpGs denoted by [[CG]] in SEQ ID NO: 5139;
278. CpGs whose cancer index value is determined are located within at least DMR 139 defined by SEQ ID NO: 5139, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least four CpG from DMR 139, and more preferably wherein the cancer index value is the mean β-value for the CpGs denoted by [[CG]] in SEQ ID NO: 5139;
279. CpGs whose cancer index value is determined are located within at least DMR 140 defined by SEQ ID NO: 5140, and wherein when the cancer index for the individual is about 0.099 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least eight CpG from DMR 140, and more preferably wherein the cancer index value is the mean β-value for the CpGs denoted by [[CG]] in SEQ ID NO: 5140;
280. CpGs whose cancer index value is determined are located within at least DMR 140 defined by SEQ ID NO: 5140, and wherein when the cancer index for the individual is less than about 0.099 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least eight CpG from DMR 140, and more preferably wherein the cancer index value is the mean β-value for the CpGs denoted by [[CG]] in SEQ ID NO: 5140;
281. CpGs whose cancer index value is determined are located within at least DMR 141 defined by SEQ ID NO: 5141, and wherein when the cancer index for the individual is about 0.044 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 141, and more preferably wherein the cancer index value is the mean β-value for the CpGs denoted by [[CG]] in SEQ ID NO: 5141;
282. CpGs whose cancer index value is determined are located within at least DMR 141 defined by SEQ ID NO: 5141, and wherein when the cancer index for the individual is less than about 0.044 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 141, and more preferably wherein the cancer index value is the mean β-value for the CpGs denoted by [[CG]] in SEQ ID NO: 5141;
283. CpGs whose cancer index value is determined are located within at least DMR 142 defined by SEQ ID NO: 5142, and wherein when the cancer index for the individual is about 0.029 or more the individual is classified as having CIN3 and/or cancer or as having a high risk of CIN3 and/or cancer development, and wherein the sensitivity of the assay is at least 70.00% and the specificity of the assay is at least 75.00%, DMR 142, and more preferably wherein the cancer index value is the mean β-value for the CpGs denoted by [[CG]] in SEQ ID NO: 5142;
284. CpGs whose cancer index value is determined are located within at least DMR 142 defined by SEQ ID NO: 5142, and wherein when the cancer index for the individual is less than about 0.029 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least four CpG from DMR 142, and more preferably wherein the cancer index value is the mean β-value for the CpGs denoted by [[CG]] in SEQ ID NO: 5142;
285. CpGs whose cancer index value is determined are located within at least DMR 143 defined by SEQ ID NO: 5143, and wherein when the cancer index for the individual is about 0.07 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least six CpG from DMR 143, and more preferably wherein the cancer index value is the mean β-value for the CpGs denoted by [[CG]] in SEQ ID NO: 5143;
286. CpGs whose cancer index value is determined are located within at least DMR 143 defined by SEQ ID NO: 5143, and wherein when the cancer index for the individual is less than about 0.07 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least six CpG from DMR 143, and more preferably wherein the cancer index value is the mean β-value for the CpGs denoted by [[CG]] in SEQ ID NO: 5143;
287. CpGs whose cancer index value is determined are located within at least DMR 144 defined by SEQ ID NO: 5144, and wherein when the cancer index for the individual is about 0.02 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least four CpG from DMR 144, and more preferably wherein the cancer index value is the mean β-value for the CpGs denoted by [[CG]] in SEQ ID NO: 5144;
288. CpGs whose cancer index value is determined are located within at least DMR 144 defined by SEQ ID NO: 5144, and wherein when the cancer index for the individual is less than about 0.02 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least four CpG from DMR 144, and more preferably wherein the cancer index value is the mean β-value for the CpGs denoted by [[CG]] in SEQ ID NO: 5144;
289. CpGs whose cancer index value is determined are located within at least DMR 145 defined by SEQ ID NO: 5145, and wherein when the cancer index for the individual is about 0.029 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least four CpG from DMR 145, and more preferably wherein the cancer index value is the mean β-value for the CpGs denoted by [[CG]] in SEQ ID NO: 5145;
290. CpGs whose cancer index value is determined are located within at least DMR 145 defined by SEQ ID NO: 5145, and wherein when the cancer index for the individual is less than about 0.029 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least four CpG from DMR 145, and more preferably wherein the cancer index value is the mean β-value for the CpGs denoted by [[CG]] in SEQ ID NO: 5145;
291. CpGs whose cancer index value is determined are located within at least DMR 146 defined by SEQ ID NO: 5146, and wherein when the cancer index for the individual is about 0.065 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least two CpG from DMR 146, and more preferably wherein the cancer index value is the mean β-value for the CpGs denoted by [[CG]] in SEQ ID NO: 5146;
292. CpGs whose cancer index value is determined are located within at least DMR 146 defined by SEQ ID NO: 5146, and wherein when the cancer index for the individual is less than about 0.065 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least two CpG from DMR 146, and more preferably wherein the cancer index value is the mean β-value for the CpGs denoted by [[CG]] in SEQ ID NO: 5146;
293. CpGs whose cancer index value is determined are located within at least DMR 147 defined by SEQ ID NO: 5147, and wherein when the cancer index for the individual is about 0.084 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least five CpG from DMR 147, and more preferably wherein the cancer index value is the mean β-value for the CpGs denoted by [[CG]] in SEQ ID NO: 5147;
294. CpGs whose cancer index value is determined are located within at least DMR 147 defined by SEQ ID NO: 5147, and wherein when the cancer index for the individual is less than about 0.084 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least five CpG from DMR 147, and more preferably wherein the cancer index value is the mean β-value for the CpGs denoted by [[CG]] in SEQ ID NO: 5147;
295. CpGs whose cancer index value is determined are located within at least DMR 148 defined by SEQ ID NO: 5148, and wherein when the cancer index for the individual is about 0.084 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least five CpG from DMR 148, and more preferably wherein the cancer index value is the mean β-value for the CpGs denoted by [[CG]] in SEQ ID NO: 5148;
296. CpGs whose cancer index value is determined are located within at least DMR 148 defined by SEQ ID NO: 5148, and wherein when the cancer index for the individual is less than about 0.084 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least five CpG from DMR 148, and more preferably wherein the cancer index value is the mean β-value for the CpGs denoted by [[CG]] in SEQ ID NO: 5148;
297. CpGs whose cancer index value is determined are located within at least DMR 149 defined by SEQ ID NO: 5149, and wherein when the cancer index for the individual is about 0.084 or more the individual is classified as having CIN3 and/or cancer or as having a high risk of CIN3 and/or cancer development, and wherein the sensitivity of preferably wherein the panel of one or more CpGs comprises at least five CpG from DMR 149, and more preferably wherein the cancer index value is the mean β-value for the CpGs denoted by [[CG]] in SEQ ID NO: 5149;
298. CpGs whose cancer index value is determined are located within at least DMR 149 defined by SEQ ID NO: 5149, and wherein when the cancer index for the individual is less than about 0.084 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least five CpG from DMR 149, and more preferably wherein the cancer index value is the mean β-value for the CpGs denoted by [[CG]] in SEQ ID NO: 5149;
299. CpGs whose cancer index value is determined are located within at least DMR 150 defined by SEQ ID NO: 5150, and wherein when the cancer index for the individual is about 0.058 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least seven CpG from DMR 150, and more preferably wherein the cancer index value is the mean β-value for the CpGs denoted by [[CG]] in SEQ ID NO: 5150;
300. CpGs whose cancer index value is determined are located within at least DMR 150 defined by SEQ ID NO: 5150, and wherein when the cancer index for the individual is less than about 0.058 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least seven CpG from DMR 150, and more preferably wherein the cancer index value is the mean β-value for the CpGs denoted by [[CG]] in SEQ ID NO: 5150;
301. CpGs whose cancer index value is determined are located within at least DMR 151 defined by SEQ ID NO: 5151, and wherein when the cancer index for the individual is about 0.058 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least seven CpG from DMR 151, and more preferably wherein the cancer index value is the mean β-value for the CpGs denoted by [[CG]] in SEQ ID NO: 5151;
302. CpGs whose cancer index value is determined are located within at least DMR 151 defined by SEQ ID NO: 5151, and wherein when the cancer index for the individual is less than about 0.058 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least seven CpG from DMR 151, and more preferably wherein the cancer index value is the mean β-value for the CpGs denoted by [[CG]] in SEQ ID NO: 5151;
303. CpGs whose cancer index value is determined are located within at least DMR 152 defined by SEQ ID NO: 5152, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 152, and more preferably wherein the cancer index value is the mean β-value for the CpGs denoted by [[CG]] in SEQ ID NO: 5152;
304. CpGs whose cancer index value is determined are located within at least DMR 152 defined by SEQ ID NO: 5152, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 152, and more preferably wherein the cancer index value is the mean β-value for the CpGs denoted by [[CG]] in SEQ ID NO: 5152;
305. CpGs whose cancer index value is determined are located within at least DMR 153 defined by SEQ ID NO: 5153, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 153, and more preferably wherein the cancer index value is the mean β-value for the CpGs denoted by [[CG]] in SEQ ID NO: 5153;
306. CpGs whose cancer index value is determined are located within at least DMR 153 defined by SEQ ID NO: 5153, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 153, and more preferably wherein the cancer index value is the mean β-value for the CpGs denoted by [[CG]] in SEQ ID NO: 5153;
307. CpGs whose cancer index value is determined are located within at least DMR 154 defined by SEQ ID NO: 5154, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 154, and more preferably wherein the cancer index value is the mean β-value for the CpGs denoted by [[CG]] in SEQ ID NO: 5154;
308. CpGs whose cancer index value is determined are located within at least DMR 154 defined by SEQ ID NO: 5154, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 154, and more preferably wherein the cancer index value is the mean β-value for the CpGs denoted by [[CG]] in SEQ ID NO: 5154;
309. CpGs whose cancer index value is determined are located within at least DMR 155 defined by SEQ ID NO: 5155, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 155, and more preferably wherein the cancer index value is the mean β-value for the CpGs denoted by [[CG]] in SEQ ID NO: 5155;
310. CpGs whose cancer index value is determined are located within at least DMR 155 defined by SEQ ID NO: 5155, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 155, and more preferably wherein the cancer index value is the mean β-value for the CpGs denoted by [[CG]] in SEQ ID NO: 5155;
311. CpGs whose cancer index value is determined are located within at least DMR 156 defined by SEQ ID NO: 5156, and wherein when the cancer index for the individual is about 0.034 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 156, and more preferably wherein the cancer index value is the mean β-value for the CpGs denoted by [[CG]] in SEQ ID NO: 5156;
312. CpGs whose cancer index value is determined are located within at least DMR 156 defined by SEQ ID NO: 5156, and wherein when the cancer index for the individual is less than about 0.034 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 156, and more preferably wherein the cancer index value is the mean β-value for the CpGs denoted by [[CG]] in SEQ ID NO: 5156;
313. CpGs whose cancer index value is determined are located within at least DMR 157 defined by SEQ ID NO: 5157, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 157, and more preferably wherein the cancer index value is the mean β-value for the CpGs denoted by [[CG]] in SEQ ID NO: 5157;
314. CpGs whose cancer index value is determined are located within at least DMR 157 defined by SEQ ID NO: 5157, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 157, and more preferably wherein the cancer index value is the mean β-value for the CpGs denoted by [[CG]] in SEQ ID NO: 5157;
315. CpGs whose cancer index value is determined are located within at least DMR 158 defined by SEQ ID NO: 5158, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 158, and more preferably wherein the cancer index value is the mean β-value for the CpGs denoted by [[CG]] in SEQ ID NO: 5158;
316. CpGs whose cancer index value is determined are located within at least DMR 158 defined by SEQ ID NO: 5158, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 158, and more preferably wherein the cancer index value is the mean β-value for the CpGs denoted by [[CG]] in SEQ ID NO: 5158;
317. CpGs whose cancer index value is determined are located within at least DMR 159 defined by SEQ ID NO: 5159, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 159, and more preferably wherein the cancer index value is the mean β-value for the CpGs denoted by [[CG]] in SEQ ID NO: 5159;
318. CpGs whose cancer index value is determined are located within at least DMR 159 defined by SEQ ID NO: 5159, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 159, and more preferably wherein the cancer index value is the mean β-value for the CpGs denoted by [[CG]] in SEQ ID NO: 5159;
319. CpGs whose cancer index value is determined are located within at least DMR 160 defined by SEQ ID NO: 5160, and wherein when the cancer index for the individual is about 0.034 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 160, and more preferably wherein the cancer index value is the mean β-value for the CpGs denoted by [[CG]] in SEQ ID NO: 5160;
320. CpGs whose cancer index value is determined are located within at least DMR 160 defined by SEQ ID NO: 5160, and wherein when the cancer index for the individual is less than about 0.034 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 160, and more preferably wherein the cancer index value is the mean β-value for the CpGs denoted by [[CG]] in SEQ ID NO: 5160;
321. CpGs whose cancer index value is determined are located within at least DMR 161 defined by SEQ ID NO: 5161, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 161, and more preferably wherein the cancer index value is the mean β-value for the CpGs denoted by [[CG]] in SEQ ID NO: 5161;
322. CpGs whose cancer index value is determined are located within at least DMR 161 defined by SEQ ID NO: 5161, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 161, and more preferably wherein the cancer index value is the mean β-value for the CpGs denoted by [[CG]] in SEQ ID NO: 5161;
323. CpGs whose cancer index value is determined are located within at least DMR 162 defined by SEQ ID NO: 5162, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 162, and more preferably wherein the cancer index value is the mean β-value for the CpGs denoted by [[CG]] in SEQ ID NO: 5162;
324. CpGs whose cancer index value is determined are located within at least DMR 162 defined by SEQ ID NO: 5162, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 162, and more preferably wherein the cancer index value is the mean β-value for the CpGs denoted by [[CG]] in SEQ ID NO: 5162;
325. CpGs whose cancer index value is determined are located within at least DMR 163 defined by SEQ ID NO: 5163, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 163, and more preferably wherein the cancer index value is the mean β-value for the CpGs denoted by [[CG]] in SEQ ID NO: 5163;
326. CpGs whose cancer index value is determined are located within at least DMR 163 defined by SEQ ID NO: 5163, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 163, and more preferably wherein the cancer index value is the mean β-value for the CpGs denoted by [[CG]] in SEQ ID NO: 5163;
327. CpGs whose cancer index value is determined are located within at least DMR 164 defined by SEQ ID NO: 5164, and wherein when the cancer index for the individual is about 0.095 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least six CpG from DMR 164, and more preferably wherein the cancer index value is the mean β-value for the CpGs denoted by [[CG]] in SEQ ID NO: 5164;
328. CpGs whose cancer index value is determined are located within at least DMR 164 defined by SEQ ID NO: 5164, and wherein when the cancer index for the individual is less than about 0.095 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least six CpG from DMR 164, and more preferably wherein the cancer index value is the mean β-value for the CpGs denoted by [[CG]] in SEQ ID NO: 5164;
329. CpGs whose cancer index value is determined are located within at least DMR 165 defined by SEQ ID NO: 5165, and wherein when the cancer index for the individual is about 0.054 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least seven CpG from DMR 165, and more preferably wherein the cancer index value is the mean β-value for the CpGs denoted by [[CG]] in SEQ ID NO: 5165;
330. CpGs whose cancer index value is determined are located within at least DMR 165 defined by SEQ ID NO: 5165, and wherein when the cancer index for the individual is less than about 0.054 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least seven CpG from DMR 165, and more preferably wherein the cancer index value is the mean β-value for the CpGs denoted by [[CG]] in SEQ ID NO: 5165;
331. CpGs whose cancer index value is determined are located within at least DMR 166 defined by SEQ ID NO: 5166, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least ten CpG from DMR 166, and more preferably wherein the cancer index value is the mean β-value for the CpGs denoted by [[CG]] in SEQ ID NO: 5166;
332. CpGs whose cancer index value is determined are located within at least DMR 166 defined by SEQ ID NO: 5166, and wherein when the cancer index for the individual is less than about 0.042 or more the individual is classified as not having cancer or as having a low risk of cancer development, and wherein the sensitivity of the assay is at least 71.00% and the specificity of the assay is at least 75.00%, preferably wherein the panel of one or more CpGs comprises at least ten CpG from DMR 166, and more preferably wherein the cancer index value is the mean β-value for the CpGs denoted by [[CG]] in SEQ ID NO: 5166;
333. CpGs whose cancer index value is determined are located within at least DMR 167 defined by SEQ ID NO: 5167, and wherein when the cancer index for the individual is about 0.054 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least seven CpG from DMR 167, and more preferably wherein the cancer index value is the mean β-value for the CpGs denoted by [[CG]] in SEQ ID NO: 5167;
334. CpGs whose cancer index value is determined are located within at least DMR 167 defined by SEQ ID NO: 5167, and wherein when the cancer index for the individual is less than about 0.054 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least seven CpG from DMR 167, and more preferably wherein the cancer index value is the mean β-value for the CpGs denoted by [[CG]] in SEQ ID NO: 5167;
335. CpGs whose cancer index value is determined are located within at least DMR 168 defined by SEQ ID NO: 5168, and wherein when the cancer index for the individual is about 0.051 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 168, and more preferably wherein the cancer index value is the mean β-value for the CpGs denoted by [[CG]] in SEQ ID NO: 5168;
336. CpGs whose cancer index value is determined are located within at least DMR 168 defined by SEQ ID NO: 5168, and wherein when the cancer index for the individual is less than about 0.051 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 168, and more preferably wherein the cancer index value is the mean β-value for the CpGs denoted by [[CG]] in SEQ ID NO: 5168;
337. CpGs whose cancer index value is determined are located within at least DMR 169 defined by SEQ ID NO: 5169, and wherein when the cancer index for the individual is about 0.051 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 169, and more preferably wherein the cancer index value is the mean β-value for the CpGs denoted by [[CG]] in SEQ ID NO: 5169;
338. CpGs whose cancer index value is determined are located within at least DMR 169 defined by SEQ ID NO: 5169, and wherein when the cancer index for the individual is less than about 0.051 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 169, and more preferably wherein the cancer index value is the mean β-value for the CpGs denoted by [[CG]] in SEQ ID NO: 5169;
339. CpGs whose cancer index value is determined are located within at least DMR 170 defined by SEQ ID NO: 5170, and wherein when the cancer index for the individual is about 0.035 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least two CpG from DMR 170, and more preferably wherein the cancer index value is the mean β-value for the CpGs denoted by [[CG]] in SEQ ID NO: 5170;
340. CpGs whose cancer index value is determined are located within at least DMR 170 defined by SEQ ID NO: 5170, and wherein when the cancer index for the individual is less than about 0.035 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least two CpG from DMR 170, and more preferably wherein the cancer index value is the mean β-value for the CpGs denoted by [[CG]] in SEQ ID NO: 5170;
341. CpGs whose cancer index value is determined are located within at least DMR 171 defined by SEQ ID NO: 5171, and wherein when the cancer index for the individual is about 0.035 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least two CpG from DMR 171, and more preferably wherein the cancer index value is the mean β-value for the CpGs denoted by [[CG]] in SEQ ID NO: 5171;
342. CpGs whose cancer index value is determined are located within at least DMR 171 defined by SEQ ID NO: 5171, and wherein when the cancer index for the individual is less than about 0.035 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least two CpG from DMR 171, and more preferably wherein the cancer index value is the mean β-value for the CpGs denoted by [[CG]] in SEQ ID NO: 5171;
343. CpGs whose cancer index value is determined are located within at least DMR 172 defined by SEQ ID NO: 5172, and wherein when the cancer index for the individual is about 0.051 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 172, and more preferably wherein the cancer index value is the mean β-value for the CpGs denoted by [[CG]] in SEQ ID NO: 5172;
344. CpGs whose cancer index value is determined are located within at least DMR 172 defined by SEQ ID NO: 5172, and wherein when the cancer index for the individual is less than about 0.051 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 172, and more preferably wherein the cancer index value is the mean β-value for the CpGs denoted by [[CG]] in SEQ ID NO: 5172;
345. CpGs whose cancer index value is determined are located within at least DMR 173 defined by SEQ ID NO: 5173, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least ten CpG from DMR 173, and more preferably wherein the cancer index value is the mean β-value for the CpGs denoted by [[CG]] in SEQ ID NO: 5173;
346. CpGs whose cancer index value is determined are located within at least DMR 173 defined by SEQ ID NO: 5173, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least ten CpG from DMR 173, and more preferably wherein the cancer index value is the mean β-value for the CpGs denoted by [[CG]] in SEQ ID NO: 5173;
347. CpGs whose cancer index value is determined are located within at least DMR 174 defined by SEQ ID NO: 5174, and wherein when the cancer index for the individual is about 0.056 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least four CpG from DMR 174, and more preferably wherein the cancer index value is the mean β-value for the CpGs denoted by [[CG]] in SEQ ID NO: 5174;
348. CpGs whose cancer index value is determined are located within at least DMR 174 defined by SEQ ID NO: 5174, and wherein when the cancer index for the individual is less than about 0.056 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least four CpG from DMR 174, and more preferably wherein the cancer index value is the mean β-value for the CpGs denoted by [[CG]] in SEQ ID NO: 5174;
349. CpGs whose cancer index value is determined are located within at least DMR 175 defined by SEQ ID NO: 5175, and wherein when the cancer index for the individual is about 0.059 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 175, and more preferably wherein the cancer index value is the mean β-value for the CpGs denoted by [[CG]] in SEQ ID NO: 5175;
350. CpGs whose cancer index value is determined are located within at least DMR 175 defined by SEQ ID NO: 5175, and wherein when the cancer index for the individual is less than about 0.059 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 175, and more preferably wherein the cancer index value is the mean β-value for the CpGs denoted by [[CG]] in SEQ ID NO: 5175;
351. CpGs whose cancer index value is determined are located within at least DMR 176 defined by SEQ ID NO: 5176, and wherein when the cancer index for the individual is about 0.059 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 176, and more preferably wherein the cancer index value is the mean β-value for the CpGs denoted by [[CG]] in SEQ ID NO: 5176;
352. CpGs whose cancer index value is determined are located within at least DMR 176 defined by SEQ ID NO: 5176, and wherein when the cancer index for the individual is less than about 0.059 the individual is classified as not having CIN3 and/or cancer or as having a low risk of CIN3 and/or cancer development, and wherein the sensitivity of the assay is at least 72.00% and the specificity of the assay is at least 75.00%, DMR 176, and more preferably wherein the cancer index value is the mean β-value for the CpGs denoted by [[CG]] in SEQ ID NO: 5176;
353. CpGs whose cancer index value is determined are located within at least DMR 177 defined by SEQ ID NO: 5177, and wherein when the cancer index for the individual is about 0.059 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 177, and more preferably wherein the cancer index value is the mean β-value for the CpGs denoted by [[CG]] in SEQ ID NO: 5177;
354. CpGs whose cancer index value is determined are located within at least DMR 177 defined by SEQ ID NO: 5177, and wherein when the cancer index for the individual is less than about 0.059 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 177, and more preferably wherein the cancer index value is the mean β-value for the CpGs denoted by [[CG]] in SEQ ID NO: 5177;
355. CpGs whose cancer index value is determined are located within at least DMR 178 defined by SEQ ID NO: 5178, and wherein when the cancer index for the individual is about 0.021 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least two CpG from DMR 178, and more preferably wherein the cancer index value is the mean β-value for the CpGs denoted by [[CG]] in SEQ ID NO: 5178;
356. CpGs whose cancer index value is determined are located within at least DMR 178 defined by SEQ ID NO: 5178, and wherein when the cancer index for the individual is less than about 0.021 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least two CpG from DMR 178, and more preferably wherein the cancer index value is the mean β-value for the CpGs denoted by [[CG]] in SEQ ID NO: 5178;
357. CpGs whose cancer index value is determined are located within at least DMR 179 defined by SEQ ID NO: 5179, and wherein when the cancer index for the individual is about 0.059 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 179, and more preferably wherein the cancer index value is the mean β-value for the CpGs denoted by [[CG]] in SEQ ID NO: 5179;
358. CpGs whose cancer index value is determined are located within at least DMR 179 defined by SEQ ID NO: 5179, and wherein when the cancer index for the individual is less than about 0.059 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 179, and more preferably wherein the cancer index value is the mean β-value for the CpGs denoted by [[CG]] in SEQ ID NO: 5179;
359. CpGs whose cancer index value is determined are located within at least DMR 180 defined by SEQ ID NO: 5180, and wherein when the cancer index for the individual is about 0.07 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least seven CpG from DMR 180, and more preferably wherein the cancer index value is the mean β-value for the CpGs denoted by [[CG]] in SEQ ID NO: 5180;
360. CpGs whose cancer index value is determined are located within at least DMR 180 defined by SEQ ID NO: 5180, and wherein when the cancer index for the individual is less than about 0.07 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least seven CpG from DMR 180, and more preferably wherein the cancer index value is the mean β-value for the CpGs denoted by [[CG]] in SEQ ID NO: 5180;
361. CpGs whose cancer index value is determined are located within at least DMR 181 defined by SEQ ID NO: 5181, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least three CpG from DMR 181, and more preferably wherein the cancer index value is the mean β-value for the CpGs denoted by [[CG]] in SEQ ID NO: 5181;
362. CpGs whose cancer index value is determined are located within at least DMR 181 defined by SEQ ID NO: 5181, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least three CpG from DMR 181, and more preferably wherein the cancer index value is the mean β-value for the CpGs denoted by [[CG]] in SEQ ID NO: 5181;
363. CpGs whose cancer index value is determined are located within at least DMR 182 defined by SEQ ID NO: 5182, and wherein when the cancer index for the individual is about 0.042 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 182, and more preferably wherein the cancer index value is the mean β-value for the CpGs denoted by [[CG]] in SEQ ID NO: 5182;
364. CpGs whose cancer index value is determined are located within at least DMR 182 defined by SEQ ID NO: 5182, and wherein when the cancer index for the individual is less than about 0.042 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 182, and more preferably wherein the cancer index value is the mean β-value for the CpGs denoted by [[CG]] in SEQ ID NO: 5182;
365. CpGs whose cancer index value is determined are located within at least DMR 183 defined by SEQ ID NO: 5183, and wherein when the cancer index for the individual is about 0.038 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least six CpG from DMR 183, and more preferably wherein the cancer index value is the mean β-value for the CpGs denoted by [[CG]] in SEQ ID NO: 5183;
366. CpGs whose cancer index value is determined are located within at least DMR 183 defined by SEQ ID NO: 5183, and wherein when the cancer index for the individual is less than about 0.038 the individual is classified as not having CIN3 and/or cancer or as having a low risk of CIN3 and/or cancer development, and wherein the sensitivity of preferably wherein the panel of one or more CpGs comprises at least six CpG from DMR 183, and more preferably wherein the cancer index value is the mean β-value for the CpGs denoted by [[CG]] in SEQ ID NO: 5183;
367. CpGs whose cancer index value is determined are located within at least DMR 184 defined by SEQ ID NO: 5184, and wherein when the cancer index for the individual is about 0.042 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 184, and more preferably wherein the cancer index value is the mean β-value for the CpGs denoted by [[CG]] in SEQ ID NO: 5184;
368. CpGs whose cancer index value is determined are located within at least DMR 184 defined by SEQ ID NO: 5184, and wherein when the cancer index for the individual is less than about 0.042 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 184, and more preferably wherein the cancer index value is the mean β-value for the CpGs denoted by [[CG]] in SEQ ID NO: 5184;
369. CpGs whose cancer index value is determined are located within at least DMR 185 defined by SEQ ID NO: 5185, and wherein when the cancer index for the individual is about 0.07 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least seven CpG from DMR 185, and more preferably wherein the cancer index value is the mean β-value for the CpGs denoted by [[CG]] in SEQ ID NO: 5185;
370. CpGs whose cancer index value is determined are located within at least DMR 185 defined by SEQ ID NO: 5185, and wherein when the cancer index for the individual is less than about 0.07 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least seven CpG from DMR 185, and more preferably wherein the cancer index value is the mean β-value for the CpGs denoted by [[CG]] in SEQ ID NO: 5185;
371. CpGs whose cancer index value is determined are located within at least DMR 186 defined by SEQ ID NO: 5186, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least three CpG from DMR 186, and more preferably wherein the cancer index value is the mean β-value for the CpGs denoted by [[CG]] in SEQ ID NO: 5186;
372. CpGs whose cancer index value is determined are located within at least DMR 186 defined by SEQ ID NO: 5186, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least three CpG from DMR 186, and more preferably wherein the cancer index value is the mean β-value for the CpGs denoted by [[CG]] in SEQ ID NO: 5186;
373. CpGs whose cancer index value is determined are located within at least DMR 187 defined by SEQ ID NO: 5187, and wherein when the cancer index for the individual is about 0.04 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least three CpG from DMR 187, and more preferably wherein the cancer index value is the mean β-value for the CpGs denoted by [[CG]] in SEQ ID NO: 5187;
374. CpGs whose cancer index value is determined are located within at least DMR 187 defined by SEQ ID NO: 5187, and wherein when the cancer index for the individual is less than about 0.04 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least three CpG from DMR 187, and more preferably wherein the cancer index value is the mean β-value for the CpGs denoted by [[CG]] in SEQ ID NO: 5187;
375. CpGs whose cancer index value is determined are located within at least DMR 188 defined by SEQ ID NO: 5188, and wherein when the cancer index for the individual is about 0.078 or more the individual is classified as having CIN3 and/or cancer or as having a high risk of CIN3 and/or cancer development, and wherein the sensitivity of the assay is at least 75.00% and the specificity of the assay is at least 75.00%, DMR 188, and more preferably wherein the cancer index value is the mean β-value for the CpGs denoted by [[CG]] in SEQ ID NO: 5188;
376. CpGs whose cancer index value is determined are located within at least DMR 188 defined by SEQ ID NO: 5188, and wherein when the cancer index for the individual is less than about 0.078 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least five CpG from DMR 188, and more preferably wherein the cancer index value is the mean β-value for the CpGs denoted by [[CG]] in SEQ ID NO: 5188;
377. CpGs whose cancer index value is determined are located within at least DMR 189 defined by SEQ ID NO: 5189, and wherein when the cancer index for the individual is about 0.038 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least six CpG from DMR 189, and more preferably wherein the cancer index value is the mean β-value for the CpGs denoted by [[CG]] in SEQ ID NO: 5189;
378. CpGs whose cancer index value is determined are located within at least DMR 189 defined by SEQ ID NO: 5189, and wherein when the cancer index for the individual is less than about 0.038 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least six CpG from DMR 189, and more preferably wherein the cancer index value is the mean β-value for the CpGs denoted by [[CG]] in SEQ ID NO: 5189;
379. CpGs whose cancer index value is determined are located within at least DMR 190 defined by SEQ ID NO: 5190, and wherein when the cancer index for the individual is about 0.019 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least one CpG from DMR 190, and more preferably wherein the cancer index value is the mean β-value for the CpGs denoted by [[CG]] in SEQ ID NO: 5190;
380. CpGs whose cancer index value is determined are located within at least DMR 190 defined by SEQ ID NO: 5190, and wherein when the cancer index for the individual is less than about 0.019 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least one CpG from DMR 190, and more preferably wherein the cancer index value is the mean β-value for the CpGs denoted by [[CG]] in SEQ ID NO: 5190;
381. CpGs whose cancer index value is determined are located within at least DMR 191 defined by SEQ ID NO: 5191, and wherein when the cancer index for the individual is about 0.037 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least seven CpG from DMR 191, and more preferably wherein the cancer index value is the mean β-value for the CpGs denoted by [[CG]] in SEQ ID NO: 5191;
382. CpGs whose cancer index value is determined are located within at least DMR 191 defined by SEQ ID NO: 5191, and wherein when the cancer index for the individual is less than about 0.037 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least seven CpG from DMR 191, and more preferably wherein the cancer index value is the mean β-value for the CpGs denoted by [[CG]] in SEQ ID NO: 5191;
383. CpGs whose cancer index value is determined are located within at least DMR 192 defined by SEQ ID NO: 5192, and wherein when the cancer index for the individual is about 0.037 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least seven CpG from DMR 192, and more preferably wherein the cancer index value is the mean β-value for the CpGs denoted by [[CG]] in SEQ ID NO: 5192;
384. CpGs whose cancer index value is determined are located within at least DMR 192 defined by SEQ ID NO: 5192, and wherein when the cancer index for the individual is less than about 0.037 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least seven CpG from DMR 192, and more preferably wherein the cancer index value is the mean β-value for the CpGs denoted by [[CG]] in SEQ ID NO: 5192;
385. CpGs whose cancer index value is determined are located within at least DMR 193 defined by SEQ ID NO: 5193, and wherein when the cancer index for the individual is about 0.029 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least four CpG from DMR 193, and more preferably wherein the cancer index value is the mean β-value for the CpGs denoted by [[CG]] in SEQ ID NO: 5193;
386. CpGs whose cancer index value is determined are located within at least DMR 193 defined by SEQ ID NO: 5193, and wherein when the cancer index for the individual is less than about 0.029 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least four CpG from DMR 193, and more preferably wherein the cancer index value is the mean β-value for the CpGs denoted by [[CG]] in SEQ ID NO: 5193;
387. CpGs whose cancer index value is determined are located within at least DMR 194 defined by SEQ ID NO: 5194, and wherein when the cancer index for the individual is about 0.054 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least two CpG from DMR 194, and more preferably wherein the cancer index value is the mean β-value for the CpGs denoted by [[CG]] in SEQ ID NO: 5194;
388. CpGs whose cancer index value is determined are located within at least DMR 194 defined by SEQ ID NO: 5194, and wherein when the cancer index for the individual is less than about 0.054 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least two CpG from DMR 194, and more preferably wherein the cancer index value is the mean β-value for the CpGs denoted by [[CG]] in SEQ ID NO: 5194;
389. CpGs whose cancer index value is determined are located within at least DMR 195 defined by SEQ ID NO: 5195, and wherein when the cancer index for the individual is about 0.031 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 195, and more preferably wherein the cancer index value is the mean β-value for the CpGs denoted by [[CG]] in SEQ ID NO: 5195;
390. CpGs whose cancer index value is determined are located within at least DMR 195 defined by SEQ ID NO: 5195, and wherein when the cancer index for the individual is less than about 0.031 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 195, and more preferably wherein the cancer index value is the mean β-value for the CpGs denoted by [[CG]] in SEQ ID NO: 5195;
391. CpGs whose cancer index value is determined are located within at least DMR 196 defined by SEQ ID NO: 5196, and wherein when the cancer index for the individual is about 0.024 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 196, and more preferably wherein the cancer index value is the mean β-value for the CpGs denoted by [[CG]] in SEQ ID NO: 5196;
392. CpGs whose cancer index value is determined are located within at least DMR 196 defined by SEQ ID NO: 5196, and wherein when the cancer index for the individual is less than about 0.024 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 196, and more preferably wherein the cancer index value is the mean β-value for the CpGs denoted by [[CG]] in SEQ ID NO: 5196;
393. CpGs whose cancer index value is determined are located within at least DMR 197 defined by SEQ ID NO: 5197, and wherein when the cancer index for the individual is about 0.03 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 197, and more preferably wherein the cancer index value is the mean β-value for the CpGs denoted by [[CG]] in SEQ ID NO: 5197;
394. CpGs whose cancer index value is determined are located within at least DMR 197 defined by SEQ ID NO: 5197, and wherein when the cancer index for the individual is less than about 0.03 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 197, and more preferably wherein the cancer index value is the mean β-value for the CpGs denoted by [[CG]] in SEQ ID NO: 5197;
395. CpGs whose cancer index value is determined are located within at least DMR 198 defined by SEQ ID NO: 5198, and wherein when the cancer index for the individual is about 0.024 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 198, and more preferably wherein the cancer index value is the mean β-value for the CpGs denoted by [[CG]] in SEQ ID NO: 5198;
396. CpGs whose cancer index value is determined are located within at least DMR 198 defined by SEQ ID NO: 5198, and wherein when the cancer index for the individual is less than about 0.024 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 198, and more preferably wherein the cancer index value is the mean β-value for the CpGs denoted by [[CG]] in SEQ ID NO: 5198;
397. CpGs whose cancer index value is determined are located within at least DMR 199 defined by SEQ ID NO: 5199, and wherein when the cancer index for the individual is about 0.03 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 199, and more preferably wherein the cancer index value is the mean β-value for the CpGs denoted by [[CG]] in SEQ ID NO: 5199;
398. CpGs whose cancer index value is determined are located within at least DMR 199 defined by SEQ ID NO: 5199, and wherein when the cancer index for the individual is less than about 0.03 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 199, and more preferably wherein the cancer index value is the mean β-value for the CpGs denoted by [[CG]] in SEQ ID NO: 5199;
399. CpGs whose cancer index value is determined are located within at least DMR 200 defined by SEQ ID NO: 5200, and wherein when the cancer index for the individual is about 0.031 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 200, and more preferably wherein the cancer index value is the mean β-value for the CpGs denoted by [[CG]] in SEQ ID NO: 5200;
400. CpGs whose cancer index value is determined are located within at least DMR 200 defined by SEQ ID NO: 5200, and wherein when the cancer index for the individual is less than about 0.031 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 200, and more preferably wherein the cancer index value is the mean β-value for the CpGs denoted by [[CG]] in SEQ ID NO: 5200;
401. CpGs whose cancer index value is determined are located within at least DMR 201 defined by SEQ ID NO: 5201, and wherein when the cancer index for the individual is about 0.031 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 201, and more preferably wherein the cancer index value is the mean β-value for the CpGs denoted by [[CG]] in SEQ ID NO: 5201;
402. CpGs whose cancer index value is determined are located within at least DMR 201 defined by SEQ ID NO: 5201, and wherein when the cancer index for the individual is less than about 0.031 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 201, and more preferably wherein the cancer index value is the mean β-value for the CpGs denoted by [[CG]] in SEQ ID NO: 5201;
403. CpGs whose cancer index value is determined are located within at least DMR 202 defined by SEQ ID NO: 5202, and wherein when the cancer index for the individual is about 0.054 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least two CpG from DMR 202, and more preferably wherein the cancer index value is the mean β-value for the CpGs denoted by [[CG]] in SEQ ID NO: 5202;
404. CpGs whose cancer index value is determined are located within at least DMR 202 defined by SEQ ID NO: 5202, and wherein when the cancer index for the individual is less than about 0.054 the individual is classified as not having CIN3 and/or cancer or as having a low risk of CIN3 and/or cancer development, and wherein the sensitivity of the assay is at least 77.00% and the specificity of the assay is at least 75.00%, preferably wherein the panel of one or more CpGs comprises at least two CpG from DMR 202, and more preferably wherein the cancer index value is the mean β-value for the CpGs denoted by [[CG]] in SEQ ID NO: 5202;
405. CpGs whose cancer index value is determined are located within at least DMR 203 defined by SEQ ID NO: 5203, and wherein when the cancer index for the individual is about 0.03 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 203, and more preferably wherein the cancer index value is the mean β-value for the CpGs denoted by [[CG]] in SEQ ID NO: 5203;
406. CpGs whose cancer index value is determined are located within at least DMR 203 defined by SEQ ID NO: 5203, and wherein when the cancer index for the individual is less than about 0.03 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 203, and more preferably wherein the cancer index value is the mean β-value for the CpGs denoted by [[CG]] in SEQ ID NO: 5203;
407. CpGs whose cancer index value is determined are located within at least DMR 204 defined by SEQ ID NO: 5204, and wherein when the cancer index for the individual is about 0.026 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 204, and more preferably wherein the cancer index value is the mean β-value for the CpGs denoted by [[CG]] in SEQ ID NO: 5204;
408. CpGs whose cancer index value is determined are located within at least DMR 204 defined by SEQ ID NO: 5204, and wherein when the cancer index for the individual is less than about 0.026 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 204, and more preferably wherein the cancer index value is the mean β-value for the CpGs denoted by [[CG]] in SEQ ID NO: 5204;
409. CpGs whose cancer index value is determined are located within at least DMR 205 defined by SEQ ID NO: 5205, and wherein when the cancer index for the individual is about 0.022 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 205, and more preferably wherein the cancer index value is the mean β-value for the CpGs denoted by [[CG]] in SEQ ID NO: 5205;
410. CpGs whose cancer index value is determined are located within at least DMR 205 defined by SEQ ID NO: 5205, and wherein when the cancer index for the individual is less than about 0.022 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 205, and more preferably wherein the cancer index value is the mean β-value for the CpGs denoted by [[CG]] in SEQ ID NO: 5205;
411. CpGs whose cancer index value is determined are located within at least DMR 206 defined by SEQ ID NO: 5206, and wherein when the cancer index for the individual is about 0.032 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least nine CpG from DMR 206, and more preferably wherein the cancer index value is the mean β-value for the CpGs denoted by [[CG]] in SEQ ID NO: 5206;
412. CpGs whose cancer index value is determined are located within at least DMR 206 defined by SEQ ID NO: 5206, and wherein when the cancer index for the individual is less than about 0.032 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least nine CpG from DMR 206, and more preferably wherein the cancer index value is the mean β-value for the CpGs denoted by [[CG]] in SEQ ID NO: 5206;
413. CpGs whose cancer index value is determined are located within at least DMR 207 defined by SEQ ID NO: 5207, and wherein when the cancer index for the individual is about 0.022 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least three CpG from DMR 207, and more preferably wherein the cancer index value is the mean β-value for the CpGs denoted by [[CG]] in SEQ ID NO: 5207;
414. CpGs whose cancer index value is determined are located within at least DMR 207 defined by SEQ ID NO: 5207, and wherein when the cancer index for the individual is less than about 0.022 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least three CpG from DMR 207, and more preferably wherein the cancer index value is the mean β-value for the CpGs denoted by [[CG]] in SEQ ID NO: 5207;
415. CpGs whose cancer index value is determined are located within at least DMR 208 defined by SEQ ID NO: 5208, and wherein when the cancer index for the individual is about 0.032 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least nine CpG from DMR 208, and more preferably wherein the cancer index value is the mean β-value for the CpGs denoted by [[CG]] in SEQ ID NO: 5208;
416. CpGs whose cancer index value is determined are located within at least DMR 208 defined by SEQ ID NO: 5208, and wherein when the cancer index for the individual is less than about 0.032 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least nine CpG from DMR 208, and more preferably wherein the cancer index value is the mean β-value for the CpGs denoted by [[CG]] in SEQ ID NO: 5208;
417. CpGs whose cancer index value is determined are located within at least DMR 209 defined by SEQ ID NO: 5209, and wherein when the cancer index for the individual is about 0.039 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least seven CpG from DMR 209, and more preferably wherein the cancer index value is the mean β-value for the CpGs denoted by [[CG]] in SEQ ID NO: 5209;
418. CpGs whose cancer index value is determined are located within at least DMR 209 defined by SEQ ID NO: 5209, and wherein when the cancer index for the individual is less than about 0.039 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least seven CpG from DMR 209, and more preferably wherein the cancer index value is the mean β-value for the CpGs denoted by [[CG]] in SEQ ID NO: 5209;
419. CpGs whose cancer index value is determined are located within at least DMR 210 defined by SEQ ID NO: 5210, and wherein when the cancer index for the individual is about 0.036 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least five CpG from DMR 210, and more preferably wherein the cancer index value is the mean β-value for the CpGs denoted by [[CG]] in SEQ ID NO: 5210;
420. CpGs whose cancer index value is determined are located within at least DMR 210 defined by SEQ ID NO: 5210, and wherein when the cancer index for the individual is less than about 0.036 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least five CpG from DMR 210, and more preferably wherein the cancer index value is the mean β-value for the CpGs denoted by [[CG]] in SEQ ID NO: 5210;
421. CpGs whose cancer index value is determined are located within at least DMR 211 defined by SEQ ID NO: 5211, and wherein when the cancer index for the individual is about 0.066 or more the individual is classified as having CIN3 and/or cancer or as having a high risk of CIN3 and/or cancer development, and wherein the sensitivity of the assay is at least 69.00% and the specificity of the assay is at least 75.00%, DMR 211, and more preferably wherein the cancer index value is the mean β-value for the CpGs denoted by [[CG]] in SEQ ID NO: 5211;
422. CpGs whose cancer index value is determined are located within at least DMR 211 defined by SEQ ID NO: 5211, and wherein when the cancer index for the individual is less than about 0.066 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least five CpG from DMR 211, and more preferably wherein the cancer index value is the mean β-value for the CpGs denoted by [[CG]] in SEQ ID NO: 5211;
423. CpGs whose cancer index value is determined are located within at least DMR 212 defined by SEQ ID NO: 5212, and wherein when the cancer index for the individual is about 0.044 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 212, and more preferably wherein the cancer index value is the mean β-value for the CpGs denoted by [[CG]] in SEQ ID NO: 5212;
424. CpGs whose cancer index value is determined are located within at least DMR 212 defined by SEQ ID NO: 5212, and wherein when the cancer index for the individual is less than about 0.044 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 212, and more preferably wherein the cancer index value is the mean β-value for the CpGs denoted by [[CG]] in SEQ ID NO: 5212;
425. CpGs whose cancer index value is determined are located within at least DMR 213 defined by SEQ ID NO: 5213, and wherein when the cancer index for the individual is about 0.044 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 213, and more preferably wherein the cancer index value is the mean β-value for the CpGs denoted by [[CG]] in SEQ ID NO: 5213;
426. CpGs whose cancer index value is determined are located within at least DMR 213 defined by SEQ ID NO: 5213, and wherein when the cancer index for the individual is less than about 0.044 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 213, and more preferably wherein the cancer index value is the mean β-value for the CpGs denoted by [[CG]] in SEQ ID NO: 5213;
427. CpGs whose cancer index value is determined are located within at least DMR 214 defined by SEQ ID NO: 5214, and wherein when the cancer index for the individual is about 0.044 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 214, and more preferably wherein the cancer index value is the mean β-value for the CpGs denoted by [[CG]] in SEQ ID NO: 5214;
428. CpGs whose cancer index value is determined are located within at least DMR 214 defined by SEQ ID NO: 5214, and wherein when the cancer index for the individual is less than about 0.044 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 214, and more preferably wherein the cancer index value is the mean β-value for the CpGs denoted by [[CG]] in SEQ ID NO: 5214;
429. CpGs whose cancer index value is determined are located within at least DMR 215 defined by SEQ ID NO: 5215, and wherein when the cancer index for the individual is about 0.042 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least two CpG from DMR 215, and more preferably wherein the cancer index value is the mean β-value for the CpGs denoted by [[CG]] in SEQ ID NO: 5215;
430. CpGs whose cancer index value is determined are located within at least DMR 215 defined by SEQ ID NO: 5215, and wherein when the cancer index for the individual is less than about 0.042 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least two CpG from DMR 215, and more preferably wherein the cancer index value is the mean β-value for the CpGs denoted by [[CG]] in SEQ ID NO: 5215;
431. CpGs whose cancer index value is determined are located within at least DMR 216 defined by SEQ ID NO: 5216, and wherein when the cancer index for the individual is about 0.066 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least five CpG from DMR 216, and more preferably wherein the cancer index value is the mean β-value for the CpGs denoted by [[CG]] in SEQ ID NO: 5216;
432. CpGs whose cancer index value is determined are located within at least DMR 216 defined by SEQ ID NO: 5216, and wherein when the cancer index for the individual is less than about 0.066 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least five CpG from DMR 216, and more preferably wherein the cancer index value is the mean β-value for the CpGs denoted by [[CG]] in SEQ ID NO: 5216;
433. CpGs whose cancer index value is determined are located within at least DMR 217 defined by SEQ ID NO: 5217, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 217, and more preferably wherein the cancer index value is the mean β-value for the CpGs denoted by [[CG]] in SEQ ID NO: 5217;
434. CpGs whose cancer index value is determined are located within at least DMR 217 defined by SEQ ID NO: 5217, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 217, and more preferably wherein the cancer index value is the mean β-value for the CpGs denoted by [[CG]] in SEQ ID NO: 5217;
435. CpGs whose cancer index value is determined are located within at least DMR 218 defined by SEQ ID NO: 5218, and wherein when the cancer index for the individual is about 0.022 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 218, and more preferably wherein the cancer index value is the mean β-value for the CpGs denoted by [[CG]] in SEQ ID NO: 5218;
436. CpGs whose cancer index value is determined are located within at least DMR 218 defined by SEQ ID NO: 5218, and wherein when the cancer index for the individual is less than about 0.022 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 218, and more preferably wherein the cancer index value is the mean β-value for the CpGs denoted by [[CG]] in SEQ ID NO: 5218;
437. CpGs whose cancer index value is determined are located within at least DMR 219 defined by SEQ ID NO: 5219, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 219, and more preferably wherein the cancer index value is the mean β-value for the CpGs denoted by [[CG]] in SEQ ID NO: 5219;
438. CpGs whose cancer index value is determined are located within at least DMR 219 defined by SEQ ID NO: 5219, and wherein when the cancer index for the individual is less than about 0.027 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 219, and more preferably wherein the cancer index value is the mean β-value for the CpGs denoted by [[CG]] in SEQ ID NO: 5219;
439. CpGs whose cancer index value is determined are located within at least DMR 220 defined by SEQ ID NO: 5220, and wherein when the cancer index for the individual is about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 220, and more preferably wherein the cancer index value is the mean β-value for the CpGs denoted by [[CG]] in SEQ ID NO: 5220;
440. CpGs whose cancer index value is determined are located within at least DMR 220 defined by SEQ ID NO: 5220, and wherein when the cancer index for the individual is less than about 0.027 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 220, and more preferably wherein the cancer index value is the mean β-value for the CpGs denoted by [[CG]] in SEQ ID NO: 5220;
441. CpGs whose cancer index value is determined are located within at least DMR 221 defined by SEQ ID NO: 5221, and wherein when the cancer index for the individual is about 0.051 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 221, and more preferably wherein the cancer index value is the mean β-value for the CpGs denoted by [[CG]] in SEQ ID NO: 5221;
442. CpGs whose cancer index value is determined are located within at least DMR 221 defined by SEQ ID NO: 5221, and wherein when the cancer index for the individual is less than about 0.051 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 221, and more preferably wherein the cancer index value is the mean β-value for the CpGs denoted by [[CG]] in SEQ ID NO: 5221;
443. CpGs whose cancer index value is determined are located within at least DMR 222 defined by SEQ ID NO: 5222, and wherein when the cancer index for the individual is about 0.044 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 222, and more preferably wherein the cancer index value is the mean β-value for the CpGs denoted by [[CG]] in SEQ ID NO: 5222;
444. CpGs whose cancer index value is determined are located within at least DMR 222 defined by SEQ ID NO: 5222, and wherein when the cancer index for the individual is less than about 0.044 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 222, and more preferably wherein the cancer index value is the mean β-value for the CpGs denoted by [[CG]] in SEQ ID NO: 5222;
445. CpGs whose cancer index value is determined are located within at least DMR 223 defined by SEQ ID NO: 5223, and wherein when the cancer index for the individual is about 0.044 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 223, and more preferably wherein the cancer index value is the mean β-value for the CpGs denoted by [[CG]] in SEQ ID NO: 5223;
446. CpGs whose cancer index value is determined are located within at least DMR 223 defined by SEQ ID NO: 5223, and wherein when the cancer index for the individual is less than about 0.044 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 223, and more preferably wherein the cancer index value is the mean β-value for the CpGs denoted by [[CG]] in SEQ ID NO: 5223;
447. CpGs whose cancer index value is determined are located within at least DMR 224 defined by SEQ ID NO: 5224, and wherein when the cancer index for the individual is about 0.049 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least nine CpG from DMR 224, and more preferably wherein the cancer index value is the mean β-value for the CpGs denoted by [[CG]] in SEQ ID NO: 5224;
448. CpGs whose cancer index value is determined are located within at least DMR 224 defined by SEQ ID NO: 5224, and wherein when the cancer index for the individual is less than about 0.049 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least nine CpG from DMR 224, and more preferably wherein the cancer index value is the mean β-value for the CpGs denoted by [[CG]] in SEQ ID NO: 5224;
449. CpGs whose cancer index value is determined are located within at least DMR 225 defined by SEQ ID NO: 5225, and wherein when the cancer index for the individual is about 0.044 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least five CpG from DMR 225, and more preferably wherein the cancer index value is the mean β-value for the CpGs denoted by [[CG]] in SEQ ID NO: 5225;
450. CpGs whose cancer index value is determined are located within at least DMR 225 defined by SEQ ID NO: 5225, and wherein when the cancer index for the individual is less than about 0.044 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least five CpG from DMR 225, and more preferably wherein the cancer index value is the mean β-value for the CpGs denoted by [[CG]] in SEQ ID NO: 5225;
451. CpGs whose cancer index value is determined are located within at least DMR 226 defined by SEQ ID NO: 5226, and wherein when the cancer index for the individual is about 0.049 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least nine CpG from DMR 226, and more preferably wherein the cancer index value is the mean β-value for the CpGs denoted by [[CG]] in SEQ ID NO: 5226;
452. CpGs whose cancer index value is determined are located within at least DMR 226 defined by SEQ ID NO: 5226, and wherein when the cancer index for the individual is less than about 0.049 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least nine CpG from DMR 226, and more preferably wherein the cancer index value is the mean β-value for the CpGs denoted by [[CG]] in SEQ ID NO: 5226;
453. CpGs whose cancer index value is determined are located within at least DMR 227 defined by SEQ ID NO: 5227, and wherein when the cancer index for the individual is about 0.033 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 227, and more preferably wherein the cancer index value is the mean β-value for the CpGs denoted by [[CG]] in SEQ ID NO: 5227;
454. CpGs whose cancer index value is determined are located within at least DMR 227 defined by SEQ ID NO: 5227, and wherein when the cancer index for the individual is less than about 0.033 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 227, and more preferably wherein the cancer index value is the mean β-value for the CpGs denoted by [[CG]] in SEQ ID NO: 5227;
455. CpGs whose cancer index value is determined are located within at least DMR 228 defined by SEQ ID NO: 5228, and wherein when the cancer index for the individual is about 0.023 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least five CpG from DMR 228, and more preferably wherein the cancer index value is the mean β-value for the CpGs denoted by [[CG]] in SEQ ID NO: 5228;
456. CpGs whose cancer index value is determined are located within at least DMR 228 defined by SEQ ID NO: 5228, and wherein when the cancer index for the individual is less than about 0.023 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least five CpG from DMR 228, and more preferably wherein the cancer index value is the mean β-value for the CpGs denoted by [[CG]] in SEQ ID NO: 5228;
457. CpGs whose cancer index value is determined are located within at least DMR 229 defined by SEQ ID NO: 5229, and wherein when the cancer index for the individual is about 0.033 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 229, and more preferably wherein the cancer index value is the mean β-value for the CpGs denoted by [[CG]] in SEQ ID NO: 5229;
458. CpGs whose cancer index value is determined are located within at least DMR 229 defined by SEQ ID NO: 5229, and wherein when the cancer index for the individual is less than about 0.033 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 229, and more preferably wherein the cancer index value is the mean β-value for the CpGs denoted by [[CG]] in SEQ ID NO: 5229;
459. CpGs whose cancer index value is determined are located within at least DMR 230 defined by SEQ ID NO: 5230, and wherein when the cancer index for the individual is about 0.033 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 230, and more preferably wherein the cancer index value is the mean β-value for the CpGs denoted by [[CG]] in SEQ ID NO: 5230;
460. CpGs whose cancer index value is determined are located within at least DMR 230 defined by SEQ ID NO: 5230, and wherein when the cancer index for the individual is less than about 0.033 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 230, and more preferably wherein the cancer index value is the mean β-value for the CpGs denoted by [[CG]] in SEQ ID NO: 5230;
461. CpGs whose cancer index value is determined are located within at least DMR 231 defined by SEQ ID NO: 5231, and wherein when the cancer index for the individual is about 0.023 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 231, and more preferably wherein the cancer index value is the mean β-value for the CpGs denoted by [[CG]] in SEQ ID NO: 5231;
462. CpGs whose cancer index value is determined are located within at least DMR 231 defined by SEQ ID NO: 5231, and wherein when the cancer index for the individual is less than about 0.023 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 231, and more preferably wherein the cancer index value is the mean β-value for the CpGs denoted by [[CG]] in SEQ ID NO: 5231;
463. CpGs whose cancer index value is determined are located within at least DMR 232 defined by SEQ ID NO: 5232, and wherein when the cancer index for the individual is about 0.079 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least nine CpG from DMR 232, and more preferably wherein the cancer index value is the mean β-value for the CpGs denoted by [[CG]] in SEQ ID NO: 5232;
464. CpGs whose cancer index value is determined are located within at least DMR 232 defined by SEQ ID NO: 5232, and wherein when the cancer index for the individual is less than about 0.079 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least nine CpG from DMR 232, and more preferably wherein the cancer index value is the mean β-value for the CpGs denoted by [[CG]] in SEQ ID NO: 5232;
465. CpGs whose cancer index value is determined are located within at least DMR 233 defined by SEQ ID NO: 5233, and wherein when the cancer index for the individual is about 0.023 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 233, and more preferably wherein the cancer index value is the mean β-value for the CpGs denoted by [[CG]] in SEQ ID NO: 5233;
466. CpGs whose cancer index value is determined are located within at least DMR 233 defined by SEQ ID NO: 5233, and wherein when the cancer index for the individual is less than about 0.023 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 233, and more preferably wherein the cancer index value is the mean β-value for the CpGs denoted by [[CG]] in SEQ ID NO: 5233;
467. CpGs whose cancer index value is determined are located within at least DMR 234 defined by SEQ ID NO: 5234, and wherein when the cancer index for the individual is about 0.04 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least two CpG from DMR 234, and more preferably wherein the cancer index value is the mean β-value for the CpGs denoted by [[CG]] in SEQ ID NO: 5234;
468. CpGs whose cancer index value is determined are located within at least DMR 234 defined by SEQ ID NO: 5234, and wherein when the cancer index for the individual is less than about 0.04 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least two CpG from DMR 234, and more preferably wherein the cancer index value is the mean β-value for the CpGs denoted by [[CG]] in SEQ ID NO: 5234;
469. CpGs whose cancer index value is determined are located within at least DMR 235 defined by SEQ ID NO: 5235, and wherein when the cancer index for the individual is about 0.051 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 235, and more preferably wherein the cancer index value is the mean β-value for the CpGs denoted by [[CG]] in SEQ ID NO: 5235;
470. CpGs whose cancer index value is determined are located within at least DMR 235 defined by SEQ ID NO: 5235, and wherein when the cancer index for the individual is less than about 0.051 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 235, and more preferably wherein the cancer index value is the mean β-value for the CpGs denoted by [[CG]] in SEQ ID NO: 5235;
471. CpGs whose cancer index value is determined are located within at least DMR 236 defined by SEQ ID NO: 5236, and wherein when the cancer index for the individual is about 0.023 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 236, and more preferably wherein the cancer index value is the mean β-value for the CpGs denoted by [[CG]] in SEQ ID NO: 5236;
472. CpGs whose cancer index value is determined are located within at least DMR 236 defined by SEQ ID NO: 5236, and wherein when the cancer index for the individual is less than about 0.023 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 236, and more preferably wherein the cancer index value is the mean β-value for the CpGs denoted by [[CG]] in SEQ ID NO: 5236;
473. CpGs whose cancer index value is determined are located within at least DMR 237 defined by SEQ ID NO: 5237, and wherein when the cancer index for the individual is about 0.033 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 237, and more preferably wherein the cancer index value is the mean β-value for the CpGs denoted by [[CG]] in SEQ ID NO: 5237;
474. CpGs whose cancer index value is determined are located within at least DMR 237 defined by SEQ ID NO: 5237, and wherein when the cancer index for the individual is less than about 0.033 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 237, and more preferably wherein the cancer index value is the mean β-value for the CpGs denoted by [[CG]] in SEQ ID NO: 5237;
475. CpGs whose cancer index value is determined are located within at least DMR 238 defined by SEQ ID NO: 5238, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least four CpG from DMR 238, and more preferably wherein the cancer index value is the mean β-value for the CpGs denoted by [[CG]] in SEQ ID NO: 5238;
476. CpGs whose cancer index value is determined are located within at least DMR 238 defined by SEQ ID NO: 5238, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least four CpG from DMR 238, and more preferably wherein the cancer index value is the mean β-value for the CpGs denoted by [[CG]] in SEQ ID NO: 5238;
477. CpGs whose cancer index value is determined are located within at least DMR 239 defined by SEQ ID NO: 5239, and wherein when the cancer index for the individual is about 0.063 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least one CpG from DMR 239, and more preferably wherein the cancer index value is the mean β-value for the CpGs denoted by [[CG]] in SEQ ID NO: 5239;
478. CpGs whose cancer index value is determined are located within at least DMR 239 defined by SEQ ID NO: 5239, and wherein when the cancer index for the individual is less than about 0.063 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least one CpG from DMR 239, and more preferably wherein the cancer index value is the mean β-value for the CpGs denoted by [[CG]] in SEQ ID NO: 5239;
479. CpGs whose cancer index value is determined are located within at least DMR 240 defined by SEQ ID NO: 5240, and wherein when the cancer index for the individual is about 0.05 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 240, and more preferably wherein the cancer index value is the mean β-value for the CpGs denoted by [[CG]] in SEQ ID NO: 5240;
480. CpGs whose cancer index value is determined are located within at least DMR 240 defined by SEQ ID NO: 5240, and wherein when the cancer index for the individual is less than about 0.05 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 240, and more preferably wherein the cancer index value is the mean β-value for the CpGs denoted by [[CG]] in SEQ ID NO: 5240;
481. CpGs whose cancer index value is determined are located within at least DMR 241 defined by SEQ ID NO: 5241, and wherein when the cancer index for the individual is about 0.035 or more the individual is classified as having CIN3 and/or cancer or as having a high risk of CIN3 and/or cancer development, and wherein the sensitivity of preferably wherein the panel of one or more CpGs comprises at least five CpG from DMR 241, and more preferably wherein the cancer index value is the mean β-value for the CpGs denoted by [[CG]] in SEQ ID NO: 5241;
482. CpGs whose cancer index value is determined are located within at least DMR 241 defined by SEQ ID NO: 5241, and wherein when the cancer index for the individual is less than about 0.035 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least five CpG from DMR 241, and more preferably wherein the cancer index value is the mean β-value for the CpGs denoted by [[CG]] in SEQ ID NO: 5241;
483. CpGs whose cancer index value is determined are located within at least DMR 242 defined by SEQ ID NO: 5242, and wherein when the cancer index for the individual is about 0.058 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least five CpG from DMR 242, and more preferably wherein the cancer index value is the mean β-value for the CpGs denoted by [[CG]] in SEQ ID NO: 5242;
484. CpGs whose cancer index value is determined are located within at least DMR 242 defined by SEQ ID NO: 5242, and wherein when the cancer index for the individual is less than about 0.058 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least five CpG from DMR 242, and more preferably wherein the cancer index value is the mean β-value for the CpGs denoted by [[CG]] in SEQ ID NO: 5242;
485. CpGs whose cancer index value is determined are located within at least DMR 243 defined by SEQ ID NO: 5243, and wherein when the cancer index for the individual is about 0.039 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 243, and more preferably wherein the cancer index value is the mean β-value for the CpGs denoted by [[CG]] in SEQ ID NO: 5243;
486. CpGs whose cancer index value is determined are located within at least DMR 243 defined by SEQ ID NO: 5243, and wherein when the cancer index for the individual is less than about 0.039 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 243, and more preferably wherein the cancer index value is the mean β-value for the CpGs denoted by [[CG]] in SEQ ID NO: 5243;
487. CpGs whose cancer index value is determined are located within at least DMR 244 defined by SEQ ID NO: 5244, and wherein when the cancer index for the individual is about 0.028 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least ten CpG from DMR 244, and more preferably wherein the cancer index value is the mean β-value for the CpGs denoted by [[CG]] in SEQ ID NO: 5244;
488. CpGs whose cancer index value is determined are located within at least DMR 244 defined by SEQ ID NO: 5244, and wherein when the cancer index for the individual is less than about 0.028 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least ten CpG from DMR 244, and more preferably wherein the cancer index value is the mean β-value for the CpGs denoted by [[CG]] in SEQ ID NO: 5244;
489. CpGs whose cancer index value is determined are located within at least DMR 245 defined by SEQ ID NO: 5245, and wherein when the cancer index for the individual is about 0.034 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 245, and more preferably wherein the cancer index value is the mean β-value for the CpGs denoted by [[CG]] in SEQ ID NO: 5245;
490. CpGs whose cancer index value is determined are located within at least DMR 245 defined by SEQ ID NO: 5245, and wherein when the cancer index for the individual is less than about 0.034 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 245, and more preferably wherein the cancer index value is the mean β-value for the CpGs denoted by [[CG]] in SEQ ID NO: 5245;
491. CpGs whose cancer index value is determined are located within at least DMR 246 defined by SEQ ID NO: 5246, and wherein when the cancer index for the individual is about 0.022 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least four CpG from DMR 246, and more preferably wherein the cancer index value is the mean β-value for the CpGs denoted by [[CG]] in SEQ ID NO: 5246;
492. CpGs whose cancer index value is determined are located within at least DMR 246 defined by SEQ ID NO: 5246, and wherein when the cancer index for the individual is less than about 0.022 the individual is classified as not having CIN3 and/or cancer or as having a low risk of CIN3 and/or cancer development, and wherein the sensitivity of the assay is at least 76.00% and the specificity of the assay is at least 75.00%, preferably wherein the panel of one or more CpGs comprises at least four CpG from DMR 246, and more preferably wherein the cancer index value is the mean β-value for the CpGs denoted by [[CG]] in SEQ ID NO: 5246;
493. CpGs whose cancer index value is determined are located within at least DMR 247 defined by SEQ ID NO: 5247, and wherein when the cancer index for the individual is about 0.039 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 247, and more preferably wherein the cancer index value is the mean β-value for the CpGs denoted by [[CG]] in SEQ ID NO: 5247;
494. CpGs whose cancer index value is determined are located within at least DMR 247 defined by SEQ ID NO: 5247, and wherein when the cancer index for the individual is less than about 0.039 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 247, and more preferably wherein the cancer index value is the mean β-value for the CpGs denoted by [[CG]] in SEQ ID NO: 5247;
495. CpGs whose cancer index value is determined are located within at least DMR 248 defined by SEQ ID NO: 5248, and wherein when the cancer index for the individual is about 0.187 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 248, and more preferably wherein the cancer index value is the mean β-value for the CpGs denoted by [[CG]] in SEQ ID NO: 5248;
496. CpGs whose cancer index value is determined are located within at least DMR 248 defined by SEQ ID NO: 5248, and wherein when the cancer index for the individual is less than about 0.187 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 248, and more preferably wherein the cancer index value is the mean β-value for the CpGs denoted by [[CG]] in SEQ ID NO: 5248;
497. CpGs whose cancer index value is determined are located within at least DMR 249 defined by SEQ ID NO: 5249, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 249, and more preferably wherein the cancer index value is the mean β-value for the CpGs denoted by [[CG]] in SEQ ID NO: 5249;
498. CpGs whose cancer index value is determined are located within at least DMR 249 defined by SEQ ID NO: 5249, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 249, and more preferably wherein the cancer index value is the mean β-value for the CpGs denoted by [[CG]] in SEQ ID NO: 5249;
499. CpGs whose cancer index value is determined are located within at least DMR 250 defined by SEQ ID NO: 5250, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 250, and more preferably wherein the cancer index value is the mean β-value for the CpGs denoted by [[CG]] in SEQ ID NO: 5250;
500. CpGs whose cancer index value is determined are located within at least DMR 250 defined by SEQ ID NO: 5250, and wherein when the cancer index for the individual is less than about 0.042 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 250, and more preferably wherein the cancer index value is the mean β-value for the CpGs denoted by [[CG]] in SEQ ID NO: 5250;
501. CpGs whose cancer index value is determined are located within at least DMR 251 defined by SEQ ID NO: 5251, and wherein when the cancer index for the individual is about 0.06 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least seven CpG from DMR 251, and more preferably wherein the cancer index value is the mean β-value for the CpGs denoted by [[CG]] in SEQ ID NO: 5251;
502. CpGs whose cancer index value is determined are located within at least DMR 251 defined by SEQ ID NO: 5251, and wherein when the cancer index for the individual is less than about 0.06 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least seven CpG from DMR 251, and more preferably wherein the cancer index value is the mean β-value for the CpGs denoted by [[CG]] in SEQ ID NO: 5251;
503. CpGs whose cancer index value is determined are located within at least DMR 252 defined by SEQ ID NO: 5252, and wherein when the cancer index for the individual is about 0.04 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least eight CpG from DMR 252, and more preferably wherein the cancer index value is the mean β-value for the CpGs denoted by [[CG]] in SEQ ID NO: 5252;
504. CpGs whose cancer index value is determined are located within at least DMR 252 defined by SEQ ID NO: 5252, and wherein when the cancer index for the individual is less than about 0.04 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least eight CpG from DMR 252, and more preferably wherein the cancer index value is the mean β-value for the CpGs denoted by [[CG]] in SEQ ID NO: 5252;
505. CpGs whose cancer index value is determined are located within at least DMR 253 defined by SEQ ID NO: 5253, and wherein when the cancer index for the individual is about 0.018 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least three CpG from DMR 253, and more preferably wherein the cancer index value is the mean β-value for the CpGs denoted by [[CG]] in SEQ ID NO: 5253;
506. CpGs whose cancer index value is determined are located within at least DMR 253 defined by SEQ ID NO: 5253, and wherein when the cancer index for the individual is less than about 0.018 the individual is classified as not having CIN3 and/or cancer or as having a low risk of CIN3 and/or cancer development, and wherein the sensitivity of the assay is at least 66.00% and the specificity of the assay is at least 75.00%, preferably wherein the panel of one or more CpGs comprises at least three CpG from DMR 253, and more preferably wherein the cancer index value is the mean β-value for the CpGs denoted by [[CG]] in SEQ ID NO: 5253;
507. CpGs whose cancer index value is determined are located within at least DMR 254 defined by SEQ ID NO: 5254, and wherein when the cancer index for the individual is about 0.026 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 254, and more preferably wherein the cancer index value is the mean β-value for the CpGs denoted by [[CG]] in SEQ ID NO: 5254;
508. CpGs whose cancer index value is determined are located within at least DMR 254 defined by SEQ ID NO: 5254, and wherein when the cancer index for the individual is less than about 0.026 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 254, and more preferably wherein the cancer index value is the mean β-value for the CpGs denoted by [[CG]] in SEQ ID NO: 5254;
509. CpGs whose cancer index value is determined are located within at least DMR 255 defined by SEQ ID NO: 5255, and wherein when the cancer index for the individual is about 0.026 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 255, and more preferably wherein the cancer index value is the mean β-value for the CpGs denoted by [[CG]] in SEQ ID NO: 5255;
510. CpGs whose cancer index value is determined are located within at least DMR 255 defined by SEQ ID NO: 5255, and wherein when the cancer index for the individual is less than about 0.026 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 255, and more preferably wherein the cancer index value is the mean β-value for the CpGs denoted by [[CG]] in SEQ ID NO: 5255;
511. CpGs whose cancer index value is determined are located within at least DMR 256 defined by SEQ ID NO: 5256, and wherein when the cancer index for the individual is about 0.051 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 256, and more preferably wherein the cancer index value is the mean β-value for the CpGs denoted by [[CG]] in SEQ ID NO: 5256;
512. CpGs whose cancer index value is determined are located within at least DMR 256 defined by SEQ ID NO: 5256, and wherein when the cancer index for the individual is less than about 0.051 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 256, and more preferably wherein the cancer index value is the mean β-value for the CpGs denoted by [[CG]] in SEQ ID NO: 5256;
513. CpGs whose cancer index value is determined are located within at least DMR 257 defined by SEQ ID NO: 5257, and wherein when the cancer index for the individual is about 0.051 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 257, and more preferably wherein the cancer index value is the mean β-value for the CpGs denoted by [[CG]] in SEQ ID NO: 5257;
514. CpGs whose cancer index value is determined are located within at least DMR 257 defined by SEQ ID NO: 5257, and wherein when the cancer index for the individual is less than about 0.051 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 257, and more preferably wherein the cancer index value is the mean β-value for the CpGs denoted by [[CG]] in SEQ ID NO: 5257;
515. CpGs whose cancer index value is determined are located within at least DMR 258 defined by SEQ ID NO: 5258, and wherein when the cancer index for the individual is about 0.051 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least two CpG from DMR 258, and more preferably wherein the cancer index value is the mean β-value for the CpGs denoted by [[CG]] in SEQ ID NO: 5258;
516. CpGs whose cancer index value is determined are located within at least DMR 258 defined by SEQ ID NO: 5258, and wherein when the cancer index for the individual is less than about 0.051 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least two CpG from DMR 258, and more preferably wherein the cancer index value is the mean β-value for the CpGs denoted by [[CG]] in SEQ ID NO: 5258;
517. CpGs whose cancer index value is determined are located within at least DMR 259 defined by SEQ ID NO: 5259, and wherein when the cancer index for the individual is about 0.091 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least two CpG from DMR 259, and more preferably wherein the cancer index value is the mean β-value for the CpGs denoted by [[CG]] in SEQ ID NO: 5259;
518. CpGs whose cancer index value is determined are located within at least DMR 259 defined by SEQ ID NO: 5259, and wherein when the cancer index for the individual is less than about 0.091 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least two CpG from DMR 259, and more preferably wherein the cancer index value is the mean β-value for the CpGs denoted by [[CG]] in SEQ ID NO: 5259;
519. CpGs whose cancer index value is determined are located within at least DMR 260 defined by SEQ ID NO: 5260, and wherein when the cancer index for the individual is about 0.044 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least seven CpG from DMR 260, and more preferably wherein the cancer index value is the mean β-value for the CpGs denoted by [[CG]] in SEQ ID NO: 5260;
520. CpGs whose cancer index value is determined are located within at least DMR 260 defined by SEQ ID NO: 5260, and wherein when the cancer index for the individual is less than about 0.044 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least seven CpG from DMR 260, and more preferably wherein the cancer index value is the mean β-value for the CpGs denoted by [[CG]] in SEQ ID NO: 5260;
521. CpGs whose cancer index value is determined are located within at least DMR 261 defined by SEQ ID NO: 5261, and wherein when the cancer index for the individual is about 0.051 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least two CpG from DMR 261, and more preferably wherein the cancer index value is the mean β-value for the CpGs denoted by [[CG]] in SEQ ID NO: 5261;
522. CpGs whose cancer index value is determined are located within at least DMR 261 defined by SEQ ID NO: 5261, and wherein when the cancer index for the individual is less than about 0.051 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least two CpG from DMR 261, and more preferably wherein the cancer index value is the mean β-value for the CpGs denoted by [[CG]] in SEQ ID NO: 5261;
523. CpGs whose cancer index value is determined are located within at least DMR 262 defined by SEQ ID NO: 5262, and wherein when the cancer index for the individual is about 0.029 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 262, and more preferably wherein the cancer index value is the mean β-value for the CpGs denoted by [[CG]] in SEQ ID NO: 5262;
524. CpGs whose cancer index value is determined are located within at least DMR 262 defined by SEQ ID NO: 5262, and wherein when the cancer index for the individual is less than about 0.029 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 262, and more preferably wherein the cancer index value is the mean β-value for the CpGs denoted by [[CG]] in SEQ ID NO: 5262;
525. CpGs whose cancer index value is determined are located within at least DMR 263 defined by SEQ ID NO: 5263, and wherein when the cancer index for the individual is about 0.039 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least four CpG from DMR 263, and more preferably wherein the cancer index value is the mean β-value for the CpGs denoted by [[CG]] in SEQ ID NO: 5263;
526. CpGs whose cancer index value is determined are located within at least DMR 263 defined by SEQ ID NO: 5263, and wherein when the cancer index for the individual is less than about 0.039 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least four CpG from DMR 263, and more preferably wherein the cancer index value is the mean β-value for the CpGs denoted by [[CG]] in SEQ ID NO: 5263;
527. CpGs whose cancer index value is determined are located within at least DMR 264 defined by SEQ ID NO: 5264, and wherein when the cancer index for the individual is about 0.047 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least six CpG from DMR 264, and more preferably wherein the cancer index value is the mean β-value for the CpGs denoted by [[CG]] in SEQ ID NO: 5264;
528. CpGs whose cancer index value is determined are located within at least DMR 264 defined by SEQ ID NO: 5264, and wherein when the cancer index for the individual is less than about 0.047 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least six CpG from DMR 264, and more preferably wherein the cancer index value is the mean β-value for the CpGs denoted by [[CG]] in SEQ ID NO: 5264;
529. CpGs whose cancer index value is determined are located within at least DMR 265 defined by SEQ ID NO: 5265, and wherein when the cancer index for the individual is about 0.046 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least three CpG from DMR 265, and more preferably wherein the cancer index value is the mean β-value for the CpGs denoted by [[CG]] in SEQ ID NO: 5265;
530. CpGs whose cancer index value is determined are located within at least DMR 265 defined by SEQ ID NO: 5265, and wherein when the cancer index for the individual is less than about 0.046 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least three CpG from DMR 265, and more preferably wherein the cancer index value is the mean β-value for the CpGs denoted by [[CG]] in SEQ ID NO: 5265;
531. CpGs whose cancer index value is determined are located within at least DMR 266 defined by SEQ ID NO: 5266, and wherein when the cancer index for the individual is about 0.023 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 266, and more preferably wherein the cancer index value is the mean β-value for the CpGs denoted by [[CG]] in SEQ ID NO: 5266;
532. CpGs whose cancer index value is determined are located within at least DMR 266 defined by SEQ ID NO: 5266, and wherein when the cancer index for the individual is less than about 0.023 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 266, and more preferably wherein the cancer index value is the mean β-value for the CpGs denoted by [[CG]] in SEQ ID NO: 5266;
533. CpGs whose cancer index value is determined are located within at least DMR 267 defined by SEQ ID NO: 5267, and wherein when the cancer index for the individual is about 0.047 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least six CpG from DMR 267, and more preferably wherein the cancer index value is the mean β-value for the CpGs denoted by [[CG]] in SEQ ID NO: 5267;
534. CpGs whose cancer index value is determined are located within at least DMR 267 defined by SEQ ID NO: 5267, and wherein when the cancer index for the individual is less than about 0.047 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least six CpG from DMR 267, and more preferably wherein the cancer index value is the mean β-value for the CpGs denoted by [[CG]] in SEQ ID NO: 5267;
535. CpGs whose cancer index value is determined are located within at least DMR 268 defined by SEQ ID NO: 5268, and wherein when the cancer index for the individual is about 0.078 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 268, and more preferably wherein the cancer index value is the mean β-value for the CpGs denoted by [[CG]] in SEQ ID NO: 5268;
536. CpGs whose cancer index value is determined are located within at least DMR 268 defined by SEQ ID NO: 5268, and wherein when the cancer index for the individual is less than about 0.078 the individual is classified as not having CIN3 and/or cancer or as having a low risk of CIN3 and/or cancer development, and wherein the sensitivity of the assay is at least 70.00% and the specificity of the assay is at least 75.00%, DMR 268, and more preferably wherein the cancer index value is the mean β-value for the CpGs denoted by [[CG]] in SEQ ID NO: 5268;
537. CpGs whose cancer index value is determined are located within at least DMR 269 defined by SEQ ID NO: 5269, and wherein when the cancer index for the individual is about 0.065 or more the individual is classified as having CIN3 and/or cancer or as having a high risk of CIN3 and/or cancer development, and wherein the sensitivity of the assay is at least 75.00% and the specificity of the assay is at least 75.00%, preferably wherein the panel of one or more CpGs comprises at least four CpG from DMR 269, and more preferably wherein the cancer index value is the mean β-value for the CpGs denoted by [[CG]] in SEQ ID NO: 5269;
538. CpGs whose cancer index value is determined are located within at least DMR 269 defined by SEQ ID NO: 5269, and wherein when the cancer index for the individual is less than about 0.065 the individual is classified as not having CIN3 and/or cancer or as having a low risk of CIN3 and/or cancer development, and wherein the sensitivity of the assay is at least 75.00% and the specificity of the assay is at least 75.00%, preferably wherein the panel of one or more CpGs comprises at least four CpG from DMR 269, and more preferably wherein the cancer index value is the mean β-value for the CpGs denoted by [[CG]] in SEQ ID NO: 5269;
539. CpGs whose cancer index value is determined are located within at least DMR 270 defined by SEQ ID NO: 5270, and wherein when the cancer index for the individual is about 0.023 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 270, and more preferably wherein the cancer index value is the mean β-value for the CpGs denoted by [[CG]] in SEQ ID NO: 5270;
540. CpGs whose cancer index value is determined are located within at least DMR 270 defined by SEQ ID NO: 5270, and wherein when the cancer index for the individual is less than about 0.023 or more the individual is classified as not having cancer or as having a high risk of cancer development, and wherein the sensitivity of the assay is at least 72.00% and the specificity of the assay is at least 75.00%, preferably wherein the panel of one or more CpGs comprises at least three CpG from DMR 270, and more preferably wherein the cancer index value is the mean β-value for the CpGs denoted by [[CG]] in SEQ ID NO: 5270;
541. CpGs whose cancer index value is determined are located within at least DMR 271 defined by SEQ ID NO: 5271, and wherein when the cancer index for the individual is about 0.023 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 271, and more preferably wherein the cancer index value is the mean β-value for the CpGs denoted by [[CG]] in SEQ ID NO: 5271;
542. CpGs whose cancer index value is determined are located within at least DMR 271 defined by SEQ ID NO: 5271, and wherein when the cancer index for the individual is less than about 0.023 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 271, and more preferably wherein the cancer index value is the mean β-value for the CpGs denoted by [[CG]] in SEQ ID NO: 5271;
543. CpGs whose cancer index value is determined are located within at least DMR 272 defined by SEQ ID NO: 5272, and wherein when the cancer index for the individual is about 0.051 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 272, and more preferably wherein the cancer index value is the mean β-value for the CpGs denoted by [[CG]] in SEQ ID NO: 5272;
544. CpGs whose cancer index value is determined are located within at least DMR 272 defined by SEQ ID NO: 5272, and wherein when the cancer index for the individual is less than about 0.051 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 272, and more preferably wherein the cancer index value is the mean β-value for the CpGs denoted by [[CG]] in SEQ ID NO: 5272;
545. CpGs whose cancer index value is determined are located within at least DMR 273 defined by SEQ ID NO: 5273, and wherein when the cancer index for the individual is about 0.051 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least seven CpG from DMR 273, and more preferably wherein the cancer index value is the mean β-value for the CpGs denoted by [[CG]] in SEQ ID NO: 5273;
546. CpGs whose cancer index value is determined are located within at least DMR 273 defined by SEQ ID NO: 5273, and wherein when the cancer index for the individual is less than about 0.051 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least seven CpG from DMR 273, and more preferably wherein the cancer index value is the mean β-value for the CpGs denoted by [[CG]] in SEQ ID NO: 5273;
547. CpGs whose cancer index value is determined are located within at least DMR 274 defined by SEQ ID NO: 5274, and wherein when the cancer index for the individual is about 0.068 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least four CpG from DMR 274, and more preferably wherein the cancer index value is the mean β-value for the CpGs denoted by [[CG]] in SEQ ID NO: 5274;
548. CpGs whose cancer index value is determined are located within at least DMR 274 defined by SEQ ID NO: 5274, and wherein when the cancer index for the individual is less than about 0.068 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least four CpG from DMR 274, and more preferably wherein the cancer index value is the mean β-value for the CpGs denoted by [[CG]] in SEQ ID NO: 5274;
549. CpGs whose cancer index value is determined are located within at least DMR 275 defined by SEQ ID NO: 5275, and wherein when the cancer index for the individual is about 0.057 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least one CpG from DMR 275, and more preferably wherein the cancer index value is the mean β-value for the CpGs denoted by [[CG]] in SEQ ID NO: 5275;
550. CpGs whose cancer index value is determined are located within at least DMR 275 defined by SEQ ID NO: 5275, and wherein when the cancer index for the individual is less than about 0.057 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least one CpG from DMR 275, and more preferably wherein the cancer index value is the mean β-value for the CpGs denoted by [[CG]] in SEQ ID NO: 5275;
551. CpGs whose cancer index value is determined are located within at least DMR 276 defined by SEQ ID NO: 5276, and wherein when the cancer index for the individual is about 0.068 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least four CpG from DMR 276, and more preferably wherein the cancer index value is the mean β-value for the CpGs denoted by [[CG]] in SEQ ID NO: 5276;
552. CpGs whose cancer index value is determined are located within at least DMR 276 defined by SEQ ID NO: 5276, and wherein when the cancer index for the individual is less than about 0.068 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least four CpG from DMR 276, and more preferably wherein the cancer index value is the mean β-value for the CpGs denoted by [[CG]] in SEQ ID NO: 5276;
553. CpGs whose cancer index value is determined are located within at least DMR 277 defined by SEQ ID NO: 5277, and wherein when the cancer index for the individual is about 0.068 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least four CpG from DMR 277, and more preferably wherein the cancer index value is the mean β-value for the CpGs denoted by [[CG]] in SEQ ID NO: 5277;
554. CpGs whose cancer index value is determined are located within at least DMR 277 defined by SEQ ID NO: 5277, and wherein when the cancer index for the individual is less than about 0.068 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least four CpG from DMR 277, and more preferably wherein the cancer index value is the mean β-value for the CpGs denoted by [[CG]] in SEQ ID NO: 5277;
555. CpGs whose cancer index value is determined are located within at least DMR 278 defined by SEQ ID NO: 5278, and wherein when the cancer index for the individual is about 0.035 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least six CpG from DMR 278, and more preferably wherein the cancer index value is the mean β-value for the CpGs denoted by [[CG]] in SEQ ID NO: 5278;
556. CpGs whose cancer index value is determined are located within at least DMR 278 defined by SEQ ID NO: 5278, and wherein when the cancer index for the individual is less than about 0.035 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least six CpG from DMR 278, and more preferably wherein the cancer index value is the mean β-value for the CpGs denoted by [[CG]] in SEQ ID NO: 5278;
557. CpGs whose cancer index value is determined are located within at least DMR 279 defined by SEQ ID NO: 5279, and wherein when the cancer index for the individual is about 0.039 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 279, and more preferably wherein the cancer index value is the mean β-value for the CpGs denoted by [[CG]] in SEQ ID NO: 5279;
558. CpGs whose cancer index value is determined are located within at least DMR 279 defined by SEQ ID NO: 5279, and wherein when the cancer index for the individual is less than about 0.039 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 279, and more preferably wherein the cancer index value is the mean β-value for the CpGs denoted by [[CG]] in SEQ ID NO: 5279;
559. CpGs whose cancer index value is determined are located within at least DMR 280 defined by SEQ ID NO: 5280, and wherein when the cancer index for the individual is about 0.068 or more the individual is classified as having CIN3 and/or cancer or as having a high risk of CIN3 and/or cancer development, and wherein the sensitivity of the assay is at least 68.00% and the specificity of the assay is at least 75.00%, DMR 280, and more preferably wherein the cancer index value is the mean β-value for the CpGs denoted by [[CG]] in SEQ ID NO: 5280;
560. CpGs whose cancer index value is determined are located within at least DMR 280 defined by SEQ ID NO: 5280, and wherein when the cancer index for the individual is less than about 0.068 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least four CpG from DMR 280, and more preferably wherein the cancer index value is the mean β-value for the CpGs denoted by [[CG]] in SEQ ID NO: 5280;
561. CpGs whose cancer index value is determined are located within at least DMR 281 defined by SEQ ID NO: 5281, and wherein when the cancer index for the individual is about 0.039 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 281, and more preferably wherein the cancer index value is the mean β-value for the CpGs denoted by [[CG]] in SEQ ID NO: 5281;
562. CpGs whose cancer index value is determined are located within at least DMR 281 defined by SEQ ID NO: 5281, and wherein when the cancer index for the individual is less than about 0.039 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 281, and more preferably wherein the cancer index value is the mean β-value for the CpGs denoted by [[CG]] in SEQ ID NO: 5281;
563. CpGs whose cancer index value is determined are located within at least DMR 282 defined by SEQ ID NO: 5282, and wherein when the cancer index for the individual is about 0.028 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least eight CpG from DMR 282, and more preferably wherein the cancer index value is the mean β-value for the CpGs denoted by [[CG]] in SEQ ID NO: 5282;
564. CpGs whose cancer index value is determined are located within at least DMR 282 defined by SEQ ID NO: 5282, and wherein when the cancer index for the individual is less than about 0.028 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least eight CpG from DMR 282, and more preferably wherein the cancer index value is the mean β-value for the CpGs denoted by [[CG]] in SEQ ID NO: 5282;
565. CpGs whose cancer index value is determined are located within at least DMR 283 defined by SEQ ID NO: 5283, and wherein when the cancer index for the individual is about 0.042 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 283, and more preferably wherein the cancer index value is the mean β-value for the CpGs denoted by [[CG]] in SEQ ID NO: 5283;
566. CpGs whose cancer index value is determined are located within at least DMR 283 defined by SEQ ID NO: 5283, and wherein when the cancer index for the individual is less than about 0.042 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 283, and more preferably wherein the cancer index value is the mean β-value for the CpGs denoted by [[CG]] in SEQ ID NO: 5283;
567. CpGs whose cancer index value is determined are located within at least DMR 284 defined by SEQ ID NO: 5284, and wherein when the cancer index for the individual is about 0.032 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least five CpG from DMR 284, and more preferably wherein the cancer index value is the mean β-value for the CpGs denoted by [[CG]] in SEQ ID NO: 5284;
568. CpGs whose cancer index value is determined are located within at least DMR 284 defined by SEQ ID NO: 5284, and wherein when the cancer index for the individual is less than about 0.032 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least five CpG from DMR 284, and more preferably wherein the cancer index value is the mean β-value for the CpGs denoted by [[CG]] in SEQ ID NO: 5284;
569. CpGs whose cancer index value is determined are located within at least DMR 285 defined by SEQ ID NO: 5285, and wherein when the cancer index for the individual is about 0.091 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least two CpG from DMR 285, and more preferably wherein the cancer index value is the mean β-value for the CpGs denoted by [[CG]] in SEQ ID NO: 5285;
570. CpGs whose cancer index value is determined are located within at least DMR 285 defined by SEQ ID NO: 5285, and wherein when the cancer index for the individual is less than about 0.091 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least two CpG from DMR 285, and more preferably wherein the cancer index value is the mean β-value for the CpGs denoted by [[CG]] in SEQ ID NO: 5285;
571. CpGs whose cancer index value is determined are located within at least DMR 286 defined by SEQ ID NO: 5286, and wherein when the cancer index for the individual is about 0.069 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least two CpG from DMR 286, and more preferably wherein the cancer index value is the mean β-value for the CpGs denoted by [[CG]] in SEQ ID NO: 5286;
572. CpGs whose cancer index value is determined are located within at least DMR 286 defined by SEQ ID NO: 5286, and wherein when the cancer index for the individual is less than about 0.069 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least two CpG from DMR 286, and more preferably wherein the cancer index value is the mean β-value for the CpGs denoted by [[CG]] in SEQ ID NO: 5286;
573. CpGs whose cancer index value is determined are located within at least DMR 287 defined by SEQ ID NO: 5287, and wherein when the cancer index for the individual is about 0.091 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least two CpG from DMR 287, and more preferably wherein the cancer index value is the mean β-value for the CpGs denoted by [[CG]] in SEQ ID NO: 5287;
574. CpGs whose cancer index value is determined are located within at least DMR 287 defined by SEQ ID NO: 5287, and wherein when the cancer index for the individual is less than about 0.091 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least two CpG from DMR 287, and more preferably wherein the cancer index value is the mean β-value for the CpGs denoted by [[CG]] in SEQ ID NO: 5287;
575. CpGs whose cancer index value is determined are located within at least DMR 288 defined by SEQ ID NO: 5288, and wherein when the cancer index for the individual is about 0.061 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least seven CpG from DMR 288, and more preferably wherein the cancer index value is the mean β-value for the CpGs denoted by [[CG]] in SEQ ID NO: 5288;
576. CpGs whose cancer index value is determined are located within at least DMR 288 defined by SEQ ID NO: 5288, and wherein when the cancer index for the individual is less than about 0.061 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least seven CpG from DMR 288, and more preferably wherein the cancer index value is the mean β-value for the CpGs denoted by [[CG]] in SEQ ID NO: 5288;
577. CpGs whose cancer index value is determined are located within at least DMR 289 defined by SEQ ID NO: 5289, and wherein when the cancer index for the individual is about 0.033 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least seven CpG from DMR 289, and more preferably wherein the cancer index value is the mean β-value for the CpGs denoted by [[CG]] in SEQ ID NO: 5289;
578. CpGs whose cancer index value is determined are located within at least DMR 289 defined by SEQ ID NO: 5289, and wherein when the cancer index for the individual is less than about 0.033 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least seven CpG from DMR 289, and more preferably wherein the cancer index value is the mean β-value for the CpGs denoted by [[CG]] in SEQ ID NO: 5289;
579. CpGs whose cancer index value is determined are located within at least DMR 290 defined by SEQ ID NO: 5290, and wherein when the cancer index for the individual is about 0.045 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least two CpG from DMR 290, and more preferably wherein the cancer index value is the mean β-value for the CpGs denoted by [[CG]] in SEQ ID NO: 5290;
580. CpGs whose cancer index value is determined are located within at least DMR 290 defined by SEQ ID NO: 5290, and wherein when the cancer index for the individual is less than about 0.045 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least two CpG from DMR 290, and more preferably wherein the cancer index value is the mean β-value for the CpGs denoted by [[CG]] in SEQ ID NO: 5290;
581. CpGs whose cancer index value is determined are located within at least DMR 291 defined by SEQ ID NO: 5291, and wherein when the cancer index for the individual is about 0.056 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least nine CpG from DMR 291, and more preferably wherein the cancer index value is the mean β-value for the CpGs denoted by [[CG]] in SEQ ID NO: 5291;
582. CpGs whose cancer index value is determined are located within at least DMR 291 defined by SEQ ID NO: 5291, and wherein when the cancer index for the individual is less than about 0.056 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least nine CpG from DMR 291, and more preferably wherein the cancer index value is the mean β-value for the CpGs denoted by [[CG]] in SEQ ID NO: 5291;
583. CpGs whose cancer index value is determined are located within at least DMR 292 defined by SEQ ID NO: 5292, and wherein when the cancer index for the individual is about 0.041 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 292, and more preferably wherein the cancer index value is the mean β-value for the CpGs denoted by [[CG]] in SEQ ID NO: 5292;
584. CpGs whose cancer index value is determined are located within at least DMR 292 defined by SEQ ID NO: 5292, and wherein when the cancer index for the individual is less than about 0.041 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 292, and more preferably wherein the cancer index value is the mean β-value for the CpGs denoted by [[CG]] in SEQ ID NO: 5292;
585. CpGs whose cancer index value is determined are located within at least DMR 293 defined by SEQ ID NO: 5293, and wherein when the cancer index for the individual is about 0.033 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least twenty-one CpG from DMR 293, and more preferably wherein the cancer index value is the mean β-value for the CpGs denoted by [[CG]] in SEQ ID NO: 5293;
586. CpGs whose cancer index value is determined are located within at least DMR 293 defined by SEQ ID NO: 5293, and wherein when the cancer index for the individual is less than about 0.033 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least twenty-one CpG from DMR 293, and more preferably wherein the cancer index value is the mean β-value for the CpGs denoted by [[CG]] in SEQ ID NO: 5293;
587. CpGs whose cancer index value is determined are located within at least DMR 294 defined by SEQ ID NO: 5294, and wherein when the cancer index for the individual is about 0.056 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 294, and more preferably wherein the cancer index value is the mean β-value for the CpGs denoted by [[CG]] in SEQ ID NO: 5294;
588. CpGs whose cancer index value is determined are located within at least DMR 294 defined by SEQ ID NO: 5294, and wherein when the cancer index for the individual is less than about 0.056 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 294, and more preferably wherein the cancer index value is the mean β-value for the CpGs denoted by [[CG]] in SEQ ID NO: 5294;
589. CpGs whose cancer index value is determined are located within at least DMR 295 defined by SEQ ID NO: 5295, and wherein when the cancer index for the individual is about 0.072 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eight CpG from DMR 295, and more preferably wherein the cancer index value is the mean β-value for the CpGs denoted by [[CG]] in SEQ ID NO: 5295;
590. CpGs whose cancer index value is determined are located within at least DMR 295 defined by SEQ ID NO: 5295, and wherein when the cancer index for the individual is less than about 0.072 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eight CpG from DMR 295, and more preferably wherein the cancer index value is the mean β-value for the CpGs denoted by [[CG]] in SEQ ID NO: 5295;
591. CpGs whose cancer index value is determined are located within at least DMR 296 defined by SEQ ID NO: 5296, and wherein when the cancer index for the individual is about 0.028 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least five CpG from DMR 296, and more preferably wherein the cancer index value is the mean β-value for the CpGs denoted by [[CG]] in SEQ ID NO: 5296;
592. CpGs whose cancer index value is determined are located within at least DMR 296 defined by SEQ ID NO: 5296, and wherein when the cancer index for the individual is less than about 0.028 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least five CpG from DMR 296, and more preferably wherein the cancer index value is the mean β-value for the CpGs denoted by [[CG]] in SEQ ID NO: 5296;
593. CpGs whose cancer index value is determined are located within at least DMR 297 defined by SEQ ID NO: 5297, and wherein when the cancer index for the individual is about 0.041 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eleven CpG from DMR 297, and more preferably wherein the cancer index value is the mean β-value for the CpGs denoted by [[CG]] in SEQ ID NO: 5297;
594. CpGs whose cancer index value is determined are located within at least DMR 297 defined by SEQ ID NO: 5297, and wherein when the cancer index for the individual is less than about 0.041 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eleven CpG from DMR 297, and more preferably wherein the cancer index value is the mean β-value for the CpGs denoted by [[CG]] in SEQ ID NO: 5297;
595. CpGs whose cancer index value is determined are located within at least DMR 298 defined by SEQ ID NO: 5298, and wherein when the cancer index for the individual is about 0.041 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 298, and more preferably wherein the cancer index value is the mean β-value for the CpGs denoted by [[CG]] in SEQ ID NO: 5298;
596. CpGs whose cancer index value is determined are located within at least DMR 298 defined by SEQ ID NO: 5298, and wherein when the cancer index for the individual is less than about 0.041 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 298, and more preferably wherein the cancer index value is the mean β-value for the CpGs denoted by [[CG]] in SEQ ID NO: 5298;
597. CpGs whose cancer index value is determined are located within at least DMR 299 defined by SEQ ID NO: 5299, and wherein when the cancer index for the individual is about 0.087 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least three CpG from DMR 299, and more preferably wherein the cancer index value is the mean β-value for the CpGs denoted by [[CG]] in SEQ ID NO: 5299;
598. CpGs whose cancer index value is determined are located within at least DMR 299 defined by SEQ ID NO: 5299, and wherein when the cancer index for the individual is less than about 0.087 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least three CpG from DMR 299, and more preferably wherein the cancer index value is the mean β-value for the CpGs denoted by [[CG]] in SEQ ID NO: 5299;
599. CpGs whose cancer index value is determined are located within at least DMR 300 defined by SEQ ID NO: 5300, and wherein when the cancer index for the individual is about 0.041 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 300, and more preferably wherein the cancer index value is the mean β-value for the CpGs denoted by [[CG]] in SEQ ID NO: 5300;
600. CpGs whose cancer index value is determined are located within at least DMR 300 defined by SEQ ID NO: 5300, and wherein when the cancer index for the individual is less than about 0.041 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 300, and more preferably wherein the cancer index value is the mean β-value for the CpGs denoted by [[CG]] in SEQ ID NO: 5300;
601. CpGs whose cancer index value is determined are located within at least DMR 301 defined by SEQ ID NO: 5301, and wherein when the cancer index for the individual is about 0.041 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eleven CpG from DMR 301, and more preferably wherein the cancer index value is the mean β-value for the CpGs denoted by [[CG]] in SEQ ID NO: 5301;
602. CpGs whose cancer index value is determined are located within at least DMR 301 defined by SEQ ID NO: 5301, and wherein when the cancer index for the individual is less than about 0.041 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eleven CpG from DMR 301, and more preferably wherein the cancer index value is the mean β-value for the CpGs denoted by [[CG]] in SEQ ID NO: 5301;
603. CpGs whose cancer index value is determined are located within at least DMR 302 defined by SEQ ID NO: 5302, and wherein when the cancer index for the individual is about 0.052 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least three CpG from DMR 302, and more preferably wherein the cancer index value is the mean β-value for the CpGs denoted by [[CG]] in SEQ ID NO: 5302;
604. CpGs whose cancer index value is determined are located within at least DMR 302 defined by SEQ ID NO: 5302, and wherein when the cancer index for the individual is less than about 0.052 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least three CpG from DMR 302, and more preferably wherein the cancer index value is the mean β-value for the CpGs denoted by [[CG]] in SEQ ID NO: 5302;
605. CpGs whose cancer index value is determined are located within at least DMR 303 defined by SEQ ID NO: 5303, and wherein when the cancer index for the individual is about 0.041 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least eleven CpG from DMR 303, and more preferably wherein the cancer index value is the mean β-value for the CpGs denoted by [[CG]] in SEQ ID NO: 5303;
606. CpGs whose cancer index value is determined are located within at least DMR 303 defined by SEQ ID NO: 5303, and wherein when the cancer index for the individual is less than about 0.041 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least eleven CpG from DMR 303, and more preferably wherein the cancer index value is the mean β-value for the CpGs denoted by [[CG]] in SEQ ID NO: 5303;
607. CpGs whose cancer index value is determined are located within at least DMR 304 defined by SEQ ID NO: 5304, and wherein when the cancer index for the individual is about 0.056 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least seven CpG from DMR 304, and more preferably wherein the cancer index value is the mean β-value for the CpGs denoted by [[CG]] in SEQ ID NO: 5304;
608. CpGs whose cancer index value is determined are located within at least DMR 304 defined by SEQ ID NO: 5304, and wherein when the cancer index for the individual is less than about 0.056 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least seven CpG from DMR 304, and more preferably wherein the cancer index value is the mean β-value for the CpGs denoted by [[CG]] in SEQ ID NO: 5304;
609. CpGs whose cancer index value is determined are located within at least DMR 305 defined by SEQ ID NO: 5305, and wherein when the cancer index for the individual is about 0.113 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least one CpG from DMR 305, and more preferably wherein the cancer index value is the mean β-value for the CpGs denoted by [[CG]] in SEQ ID NO: 5305;
610. CpGs whose cancer index value is determined are located within at least DMR 305 defined by SEQ ID NO: 5305, and wherein when the cancer index for the individual is less than about 0.113 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least one CpG from DMR 305, and more preferably wherein the cancer index value is the mean β-value for the CpGs denoted by [[CG]] in SEQ ID NO: 5305;
611. CpGs whose cancer index value is determined are located within at least DMR 306 defined by SEQ ID NO: 5306, and wherein when the cancer index for the individual is about 0.061 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least six CpG from DMR 306, and more preferably wherein the cancer index value is the mean β-value for the CpGs denoted by [[CG]] in SEQ ID NO: 5306;
612. CpGs whose cancer index value is determined are located within at least DMR 306 defined by SEQ ID NO: 5306, and wherein when the cancer index for the individual is less than about 0.061 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least six CpG from DMR 306, and more preferably wherein the cancer index value is the mean β-value for the CpGs denoted by [[CG]] in SEQ ID NO: 5306;
613. CpGs whose cancer index value is determined are located within at least DMR 307 defined by SEQ ID NO: 5307, and wherein when the cancer index for the individual is about 0.045 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least twenty-three CpG from DMR 307, and more preferably wherein the cancer index value is the mean β-value for the CpGs denoted by [[CG]] in SEQ ID NO: 5307;
614. CpGs whose cancer index value is determined are located within at least DMR 307 defined by SEQ ID NO: 5307, and wherein when the cancer index for the individual is less than about 0.045 the individual is classified as not having CIN3 and/or cancer or as having a low risk of CIN3 and/or cancer development, and wherein the sensitivity of the assay is at least 66.00% and the specificity of the assay is at least 75.00%, preferably wherein the panel of one or more CpGs comprises at least twenty-three CpG from DMR 307, and more preferably wherein the cancer index value is the mean β-value for the CpGs denoted by [[CG]] in SEQ ID NO: 5307;
615. CpGs whose cancer index value is determined are located within at least DMR 308 defined by SEQ ID NO: 5308, and wherein when the cancer index for the individual is about 0.037 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 308, and more preferably wherein the cancer index value is the mean β-value for the CpGs denoted by [[CG]] in SEQ ID NO: 5308;
616. CpGs whose cancer index value is determined are located within at least DMR 308 defined by SEQ ID NO: 5308, and wherein when the cancer index for the individual is less than about 0.037 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 308, and more preferably wherein the cancer index value is the mean β-value for the CpGs denoted by [[CG]] in SEQ ID NO: 5308;
617. CpGs whose cancer index value is determined are located within at least DMR 309 defined by SEQ ID NO: 5309, and wherein when the cancer index for the individual is about 0.032 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least seven CpG from DMR 309, and more preferably wherein the cancer index value is the mean β-value for the CpGs denoted by [[CG]] in SEQ ID NO: 5309;
618. CpGs whose cancer index value is determined are located within at least DMR 309 defined by SEQ ID NO: 5309, and wherein when the cancer index for the individual is less than about 0.032 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least seven CpG from DMR 309, and more preferably wherein the cancer index value is the mean β-value for the CpGs denoted by [[CG]] in SEQ ID NO: 5309;
619. CpGs whose cancer index value is determined are located within at least DMR 310 defined by SEQ ID NO: 5310, and wherein when the cancer index for the individual is about 0.037 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least three CpG from DMR 310, and more preferably wherein the cancer index value is the mean β-value for the CpGs denoted by [[CG]] in SEQ ID NO: 5310;
620. CpGs whose cancer index value is determined are located within at least DMR 310 defined by SEQ ID NO: 5310, and wherein when the cancer index for the individual is less than about 0.037 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least three CpG from DMR 310, and more preferably wherein the cancer index value is the mean β-value for the CpGs denoted by [[CG]] in SEQ ID NO: 5310;
621. CpGs whose cancer index value is determined are located within at least DMR 311 defined by SEQ ID NO: 5311, and wherein when the cancer index for the individual is about 0.043 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least ten CpG from DMR 311, and more preferably wherein the cancer index value is the mean β-value for the CpGs denoted by [[CG]] in SEQ ID NO: 5311;
622. CpGs whose cancer index value is determined are located within at least DMR 311 defined by SEQ ID NO: 5311, and wherein when the cancer index for the individual is less than about 0.043 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least ten CpG from DMR 311, and more preferably wherein the cancer index value is the mean β-value for the CpGs denoted by [[CG]] in SEQ ID NO: 5311;
623. CpGs whose cancer index value is determined are located within at least DMR 312 defined by SEQ ID NO: 5312, and wherein when the cancer index for the individual is about 0.043 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least ten CpG from DMR 312, and more preferably wherein the cancer index value is the mean β-value for the CpGs denoted by [[CG]] in SEQ ID NO: 5312;
624. CpGs whose cancer index value is determined are located within at least DMR 312 defined by SEQ ID NO: 5312, and wherein when the cancer index for the individual is less than about 0.043 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least ten CpG from DMR 312, and more preferably wherein the cancer index value is the mean β-value for the CpGs denoted by [[CG]] in SEQ ID NO: 5312;
625. CpGs whose cancer index value is determined are located within at least DMR 313 defined by SEQ ID NO: 5313, and wherein when the cancer index for the individual is about 0.037 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 313, and more preferably wherein the cancer index value is the mean β-value for the CpGs denoted by [[CG]] in SEQ ID NO: 5313;
626. CpGs whose cancer index value is determined are located within at least DMR 313 defined by SEQ ID NO: 5313, and wherein when the cancer index for the individual is less than about 0.037 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 313, and more preferably wherein the cancer index value is the mean β-value for the CpGs denoted by [[CG]] in SEQ ID NO: 5313;
627. CpGs whose cancer index value is determined are located within at least DMR 314 defined by SEQ ID NO: 5314, and wherein when the cancer index for the individual is about 0.041 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least six CpG from DMR 314, and more preferably wherein the cancer index value is the mean β-value for the CpGs denoted by [[CG]] in SEQ ID NO: 5314;
628. CpGs whose cancer index value is determined are located within at least DMR 314 defined by SEQ ID NO: 5314, and wherein when the cancer index for the individual is less than about 0.041 the individual is classified as not having CIN3 and/or cancer or as having a low risk of CIN3 and/or cancer development, and wherein the sensitivity of the assay is at least 74.00% and the specificity of the assay is at least 75.00%, DMR 314, and more preferably wherein the cancer index value is the mean β-value for the CpGs denoted by [[CG]] in SEQ ID NO: 5314;
629. CpGs whose cancer index value is determined are located within at least DMR 315 defined by SEQ ID NO: 5315, and wherein when the cancer index for the individual is about 0.035 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least twenty-two CpG from DMR 315, and more preferably wherein the cancer index value is the mean β-value for the CpGs denoted by [[CG]] in SEQ ID NO: 5315;
630. CpGs whose cancer index value is determined are located within at least DMR 315 defined by SEQ ID NO: 5315, and wherein when the cancer index for the individual is less than about 0.035 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least twenty-two CpG from DMR 315, and more preferably wherein the cancer index value is the mean β-value for the CpGs denoted by [[CG]] in SEQ ID NO: 5315;
631. CpGs whose cancer index value is determined are located within at least DMR 316 defined by SEQ ID NO: 5316, and wherein when the cancer index for the individual is about 0.037 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 316, and more preferably wherein the cancer index value is the mean β-value for the CpGs denoted by [[CG]] in SEQ ID NO: 5316;
632. CpGs whose cancer index value is determined are located within at least DMR 316 defined by SEQ ID NO: 5316, and wherein when the cancer index for the individual is less than about 0.037 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 316, and more preferably wherein the cancer index value is the mean β-value for the CpGs denoted by [[CG]] in SEQ ID NO: 5316;
633. CpGs whose cancer index value is determined are located within at least DMR 317 defined by SEQ ID NO: 5317, and wherein when the cancer index for the individual is about 0.035 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least twenty-two CpG from DMR 317, and more preferably wherein the cancer index value is the mean β-value for the CpGs denoted by [[CG]] in SEQ ID NO: 5317;
634. CpGs whose cancer index value is determined are located within at least DMR 317 defined by SEQ ID NO: 5317, and wherein when the cancer index for the individual is less than about 0.035 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least twenty-two CpG from DMR 317, and more preferably wherein the cancer index value is the mean β-value for the CpGs denoted by [[CG]] in SEQ ID NO: 5317;
635. CpGs whose cancer index value is determined are located within at least DMR 318 defined by SEQ ID NO: 5318, and wherein when the cancer index for the individual is about 0.027 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least five CpG from DMR 318, and more preferably wherein the cancer index value is the mean β-value for the CpGs denoted by [[CG]] in SEQ ID NO: 5318;
636. CpGs whose cancer index value is determined are located within at least DMR 318 defined by SEQ ID NO: 5318, and wherein when the cancer index for the individual is less than about 0.027 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least five CpG from DMR 318, and more preferably wherein the cancer index value is the mean β-value for the CpGs denoted by [[CG]] in SEQ ID NO: 5318;
637. CpGs whose cancer index value is determined are located within at least DMR 319 defined by SEQ ID NO: 5319, and wherein when the cancer index for the individual is about 0.029 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 319, and more preferably wherein the cancer index value is the mean β-value for the CpGs denoted by [[CG]] in SEQ ID NO: 5319;
638. CpGs whose cancer index value is determined are located within at least DMR 319 defined by SEQ ID NO: 5319, and wherein when the cancer index for the individual is less than about 0.029 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 319, and more preferably wherein the cancer index value is the mean β-value for the CpGs denoted by [[CG]] in SEQ ID NO: 5319;
639. CpGs whose cancer index value is determined are located within at least DMR 320 defined by SEQ ID NO: 5320, and wherein when the cancer index for the individual is about 0.029 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 320, and more preferably wherein the cancer index value is the mean β-value for the CpGs denoted by [[CG]] in SEQ ID NO: 5320;
640. CpGs whose cancer index value is determined are located within at least DMR 320 defined by SEQ ID NO: 5320, and wherein when the cancer index for the individual is less than about 0.029 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 320, and more preferably wherein the cancer index value is the mean β-value for the CpGs denoted by [[CG]] in SEQ ID NO: 5320;
641. CpGs whose cancer index value is determined are located within at least DMR 321 defined by SEQ ID NO: 5321, and wherein when the cancer index for the individual is about 0.055 or more the individual is classified as having CIN3 and/or cancer or as having a high risk of CIN3 and/or cancer development, and wherein the sensitivity of the assay is at least 63.00% and the specificity of the assay is at least 75.00%, preferably wherein the panel of one or more CpGs comprises at least four CpG from DMR 321, and more preferably wherein the cancer index value is the mean β-value for the CpGs denoted by [[CG]] in SEQ ID NO: 5321;
642. CpGs whose cancer index value is determined are located within at least DMR 321 defined by SEQ ID NO: 5321, and wherein when the cancer index for the individual is less than about 0.055 the individual is classified as not having CIN3 and/or cancer or as having a low risk of CIN3 and/or cancer development, and wherein the sensitivity of the assay is at least 63.00% and the specificity of the assay is at least 75.00%, preferably wherein the panel of one or more CpGs comprises at least four CpG from DMR 321, and more preferably wherein the cancer index value is the mean β-value for the CpGs denoted by [[CG]] in SEQ ID NO: 5321;
643. CpGs whose cancer index value is determined are located within at least DMR 322 defined by SEQ ID NO: 5322, and wherein when the cancer index for the individual is about 0.055 or more the individual is classified as having CIN3 and/or cancer or as having a high risk of CIN3 and/or cancer development, and wherein the sensitivity of the assay is at least 63.00% and the specificity of the assay is at least 75.00%, preferably wherein the panel of one or more CpGs comprises at least four CpG from DMR 322, and more preferably wherein the cancer index value is the mean β-value for the CpGs denoted by [[CG]] in SEQ ID NO: 5322;
644. CpGs whose cancer index value is determined are located within at least DMR 322 defined by SEQ ID NO: 5322, and wherein when the cancer index for the individual is less than about 0.055 the individual is classified as not having CIN3 and/or cancer or as having a low risk of CIN3 and/or cancer development, and wherein the sensitivity of the assay is at least 63.00% and the specificity of the assay is at least 75.00%, preferably wherein the panel of one or more CpGs comprises at least four CpG from DMR 322, and more preferably wherein the cancer index value is the mean β-value for the CpGs denoted by [[CG]] in SEQ ID NO: 5322;
645. CpGs whose cancer index value is determined are located within at least DMR 323 defined by SEQ ID NO: 5323, and wherein when the cancer index for the individual is about 0.029 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least three CpG from DMR 323, and more preferably wherein the cancer index value is the mean β-value for the CpGs denoted by [[CG]] in SEQ ID NO: 5323;
646. CpGs whose cancer index value is determined are located within at least DMR 323 defined by SEQ ID NO: 5323, and wherein when the cancer index for the individual is less than about 0.029 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least three CpG from DMR 323, and more preferably wherein the cancer index value is the mean β-value for the CpGs denoted by [[CG]] in SEQ ID NO: 5323;
647. CpGs whose cancer index value is determined are located within at least DMR 324 defined by SEQ ID NO: 5324, and wherein when the cancer index for the individual is about 0.022 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least four CpG from DMR 324, and more preferably wherein the cancer index value is the mean β-value for the CpGs denoted by [[CG]] in SEQ ID NO: 5324;
648. CpGs whose cancer index value is determined are located within at least DMR 324 defined by SEQ ID NO: 5324, and wherein when the cancer index for the individual is less than about 0.022 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least four CpG from DMR 324, and more preferably wherein the cancer index value is the mean β-value for the CpGs denoted by [[CG]] in SEQ ID NO: 5324;
649. CpGs whose cancer index value is determined are located within at least DMR 325 defined by SEQ ID NO: 5325, and wherein when the cancer index for the individual is about 0.122 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least three CpG from DMR 325, and more preferably wherein the cancer index value is the mean β-value for the CpGs denoted by [[CG]] in SEQ ID NO: 5325;
650. CpGs whose cancer index value is determined are located within at least DMR 325 defined by SEQ ID NO: 5325, and wherein when the cancer index for the individual is less than about 0.122 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least three CpG from DMR 325, and more preferably wherein the cancer index value is the mean β-value for the CpGs denoted by [[CG]] in SEQ ID NO: 5325;
651. CpGs whose cancer index value is determined are located within at least DMR 326 defined by SEQ ID NO: 5326, and wherein when the cancer index for the individual is about 0.071 or more the individual is classified as having CIN3 and/or cancer or as having a high risk of CIN3 and/or cancer development, and wherein the sensitivity of the assay is at least 70.00% and the specificity of the assay is at least 75.00%, DMR 326, and more preferably wherein the cancer index value is the mean β-value for the CpGs denoted by [[CG]] in SEQ ID NO: 5326;
652. CpGs whose cancer index value is determined are located within at least DMR 326 defined by SEQ ID NO: 5326, and wherein when the cancer index for the individual is less than about 0.071 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 326, and more preferably wherein the cancer index value is the mean β-value for the CpGs denoted by [[CG]] in SEQ ID NO: 5326;
653. CpGs whose cancer index value is determined are located within at least DMR 327 defined by SEQ ID NO: 5327, and wherein when the cancer index for the individual is about 0.071 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 327, and more preferably wherein the cancer index value is the mean β-value for the CpGs denoted by [[CG]] in SEQ ID NO: 5327;
654. CpGs whose cancer index value is determined are located within at least DMR 327 defined by SEQ ID NO: 5327, and wherein when the cancer index for the individual is less than about 0.071 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 327, and more preferably wherein the cancer index value is the mean β-value for the CpGs denoted by [[CG]] in SEQ ID NO: 5327;
655. CpGs whose cancer index value is determined are located within at least DMR 328 defined by SEQ ID NO: 5328, and wherein when the cancer index for the individual is about 0.034 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least twenty-one CpG from DMR 328, and more preferably wherein the cancer index value is the mean β-value for the CpGs denoted by [[CG]] in SEQ ID NO: 5328;
656. CpGs whose cancer index value is determined are located within at least DMR 328 defined by SEQ ID NO: 5328, and wherein when the cancer index for the individual is less than about 0.034 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least twenty-one CpG from DMR 328, and more preferably wherein the cancer index value is the mean β-value for the CpGs denoted by [[CG]] in SEQ ID NO: 5328;
657. CpGs whose cancer index value is determined are located within at least DMR 329 defined by SEQ ID NO: 5329, and wherein when the cancer index for the individual is about 0.066 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 329, and more preferably wherein the cancer index value is the mean β-value for the CpGs denoted by [[CG]] in SEQ ID NO: 5329;
658. CpGs whose cancer index value is determined are located within at least DMR 329 defined by SEQ ID NO: 5329, and wherein when the cancer index for the individual is less than about 0.066 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least five CpG from DMR 329, and more preferably wherein the cancer index value is the mean β-value for the CpGs denoted by [[CG]] in SEQ ID NO: 5329;
659. CpGs whose cancer index value is determined are located within at least DMR 330 defined by SEQ ID NO: 5330, and wherein when the cancer index for the individual is about 0.122 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least three CpG from DMR 330, and more preferably wherein the cancer index value is the mean β-value for the CpGs denoted by [[CG]] in SEQ ID NO: 5330;
660. CpGs whose cancer index value is determined are located within at least DMR 330 defined by SEQ ID NO: 5330, and wherein when the cancer index for the individual is less than about 0.122 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least three CpG from DMR 330, and more preferably wherein the cancer index value is the mean β-value for the CpGs denoted by [[CG]] in SEQ ID NO: 5330;
661. CpGs whose cancer index value is determined are located within at least DMR 331 defined by SEQ ID NO: 5331, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 331, and more preferably wherein the cancer index value is the mean β-value for the CpGs denoted by [[CG]] in SEQ ID NO: 5331;
662. CpGs whose cancer index value is determined are located within at least DMR 331 defined by SEQ ID NO: 5331, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 331, and more preferably wherein the cancer index value is the mean β-value for the CpGs denoted by [[CG]] in SEQ ID NO: 5331;
663. CpGs whose cancer index value is determined are located within at least DMR 332 defined by SEQ ID NO: 5332, and wherein when the cancer index for the individual is about 0.071 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 332, and more preferably wherein the cancer index value is the mean β-value for the CpGs denoted by [[CG]] in SEQ ID NO: 5332;
664. CpGs whose cancer index value is determined are located within at least DMR 332 defined by SEQ ID NO: 5332, and wherein when the cancer index for the individual is less than about 0.071 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 332, and more preferably wherein the cancer index value is the mean β-value for the CpGs denoted by [[CG]] in SEQ ID NO: 5332;
665. CpGs whose cancer index value is determined are located within at least DMR 333 defined by SEQ ID NO: 5333, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 333, and more preferably wherein the cancer index value is the mean β-value for the CpGs denoted by [[CG]] in SEQ ID NO: 5333;
666. CpGs whose cancer index value is determined are located within at least DMR 333 defined by SEQ ID NO: 5333, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 333, and more preferably wherein the cancer index value is the mean β-value for the CpGs denoted by [[CG]] in SEQ ID NO: 5333;
667. CpGs whose cancer index value is determined are located within at least DMR 334 defined by SEQ ID NO: 5334, and wherein when the cancer index for the individual is about 0.04 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least five CpG from DMR 334, and more preferably wherein the cancer index value is the mean β-value for the CpGs denoted by [[CG]] in SEQ ID NO: 5334;
668. CpGs whose cancer index value is determined are located within at least DMR 334 defined by SEQ ID NO: 5334, and wherein when the cancer index for the individual is less than about 0.04 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least five CpG from DMR 334, and more preferably wherein the cancer index value is the mean β-value for the CpGs denoted by [[CG]] in SEQ ID NO: 5334;
669. CpGs whose cancer index value is determined are located within at least DMR 335 defined by SEQ ID NO: 5335, and wherein when the cancer index for the individual is about 0.04 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least five CpG from DMR 335, and more preferably wherein the cancer index value is the mean β-value for the CpGs denoted by [[CG]] in SEQ ID NO: 5335;
670. CpGs whose cancer index value is determined are located within at least DMR 335 defined by SEQ ID NO: 5335, and wherein when the cancer index for the individual is less than about 0.04 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least five CpG from DMR 335, and more preferably wherein the cancer index value is the mean β-value for the CpGs denoted by [[CG]] in SEQ ID NO: 5335;
671. CpGs whose cancer index value is determined are located within at least DMR 336 defined by SEQ ID NO: 5336, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 336, and more preferably wherein the cancer index value is the mean β-value for the CpGs denoted by [[CG]] in SEQ ID NO: 5336;
672. CpGs whose cancer index value is determined are located within at least DMR 336 defined by SEQ ID NO: 5336, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 336, and more preferably wherein the cancer index value is the mean β-value for the CpGs denoted by [[CG]] in SEQ ID NO: 5336;
673. CpGs whose cancer index value is determined are located within at least DMR 337 defined by SEQ ID NO: 5337, and wherein when the cancer index for the individual is about 0.234 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least ten CpG from DMR 337, and more preferably wherein the cancer index value is the mean β-value for the CpGs denoted by [[CG]] in SEQ ID NO: 5337;
674. CpGs whose cancer index value is determined are located within at least DMR 337 defined by SEQ ID NO: 5337, and wherein when the cancer index for the individual is less than about 0.234 the individual is classified as not having CIN3 and/or cancer or as having a low risk of CIN3 and/or cancer development, and wherein the sensitivity of the assay is at least 68.00% and the specificity of the assay is at least 75.00%, DMR 337, and more preferably wherein the cancer index value is the mean β-value for the CpGs denoted by [[CG]] in SEQ ID NO: 5337;
675. CpGs whose cancer index value is determined are located within at least DMR 338 defined by SEQ ID NO: 5338, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 338, and more preferably wherein the cancer index value is the mean β-value for the CpGs denoted by [[CG]] in SEQ ID NO: 5338;
676. CpGs whose cancer index value is determined are located within at least DMR 338 defined by SEQ ID NO: 5338, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 338, and more preferably wherein the cancer index value is the mean β-value for the CpGs denoted by [[CG]] in SEQ ID NO: 5338;
677. CpGs whose cancer index value is determined are located within at least DMR 339 defined by SEQ ID NO: 5339, and wherein when the cancer index for the individual is about 0.234 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least ten CpG from DMR 339, and more preferably wherein the cancer index value is the mean β-value for the CpGs denoted by [[CG]] in SEQ ID NO: 5339;
678. CpGs whose cancer index value is determined are located within at least DMR 339 defined by SEQ ID NO: 5339, and wherein when the cancer index for the individual is less than about 0.234 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least ten CpG from DMR 339, and more preferably wherein the cancer index value is the mean β-value for the CpGs denoted by [[CG]] in SEQ ID NO: 5339;
679. CpGs whose cancer index value is determined are located within at least DMR 340 defined by SEQ ID NO: 5340, and wherein when the cancer index for the individual is about 0.234 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least ten CpG from DMR 340, and more preferably wherein the cancer index value is the mean β-value for the CpGs denoted by [[CG]] in SEQ ID NO: 5340;
680. CpGs whose cancer index value is determined are located within at least DMR 340 defined by SEQ ID NO: 5340, and wherein when the cancer index for the individual is less than about 0.234 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least ten CpG from DMR 340, and more preferably wherein the cancer index value is the mean β-value for the CpGs denoted by [[CG]] in SEQ ID NO: 5340;
681. CpGs whose cancer index value is determined are located within at least DMR 341 defined by SEQ ID NO: 5341, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 341, and more preferably wherein the cancer index value is the mean β-value for the CpGs denoted by [[CG]] in SEQ ID NO: 5341;
682. CpGs whose cancer index value is determined are located within at least DMR 341 defined by SEQ ID NO: 5341, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 341, and more preferably wherein the cancer index value is the mean β-value for the CpGs denoted by [[CG]] in SEQ ID NO: 5341;
683. CpGs whose cancer index value is determined are located within at least DMR 342 defined by SEQ ID NO: 5342, and wherein when the cancer index for the individual is about 0.054 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least six CpG from DMR 342, and more preferably wherein the cancer index value is the mean β-value for the CpGs denoted by [[CG]] in SEQ ID NO: 5342;
684. CpGs whose cancer index value is determined are located within at least DMR 342 defined by SEQ ID NO: 5342, and wherein when the cancer index for the individual is less than about 0.054 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least six CpG from DMR 342, and more preferably wherein the cancer index value is the mean β-value for the CpGs denoted by [[CG]] in SEQ ID NO: 5342;
685. CpGs whose cancer index value is determined are located within at least DMR 343 defined by SEQ ID NO: 5343, and wherein when the cancer index for the individual is about 0.033 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least two CpG from DMR 343, and more preferably wherein the cancer index value is the mean β-value for the CpGs denoted by [[CG]] in SEQ ID NO: 5343;
686. CpGs whose cancer index value is determined are located within at least DMR 343 defined by SEQ ID NO: 5343, and wherein when the cancer index for the individual is less than about 0.033 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least two CpG from DMR 343, and more preferably wherein the cancer index value is the mean β-value for the CpGs denoted by [[CG]] in SEQ ID NO: 5343;
687. CpGs whose cancer index value is determined are located within at least DMR 344 defined by SEQ ID NO: 5344, and wherein when the cancer index for the individual is about 0.059 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 344, and more preferably wherein the cancer index value is the mean β-value for the CpGs denoted by [[CG]] in SEQ ID NO: 5344;
688. CpGs whose cancer index value is determined are located within at least DMR 344 defined by SEQ ID NO: 5344, and wherein when the cancer index for the individual is less than about 0.059 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 344, and more preferably wherein the cancer index value is the mean β-value for the CpGs denoted by [[CG]] in SEQ ID NO: 5344;
689. CpGs whose cancer index value is determined are located within at least DMR 345 defined by SEQ ID NO: 5345, and wherein when the cancer index for the individual is about 0.049 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 345, and more preferably wherein the cancer index value is the mean β-value for the CpGs denoted by [[CG]] in SEQ ID NO: 5345;
690. CpGs whose cancer index value is determined are located within at least DMR 345 defined by SEQ ID NO: 5345, and wherein when the cancer index for the individual is less than about 0.049 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 345, and more preferably wherein the cancer index value is the mean β-value for the CpGs denoted by [[CG]] in SEQ ID NO: 5345;
691. CpGs whose cancer index value is determined are located within at least DMR 346 defined by SEQ ID NO: 5346, and wherein when the cancer index for the individual is about 0.06 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least four CpG from DMR 346, and more preferably wherein the cancer index value is the mean β-value for the CpGs denoted by [[CG]] in SEQ ID NO: 5346;
692. CpGs whose cancer index value is determined are located within at least DMR 346 defined by SEQ ID NO: 5346, and wherein when the cancer index for the individual is less than about 0.06 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least four CpG from DMR 346, and more preferably wherein the cancer index value is the mean β-value for the CpGs denoted by [[CG]] in SEQ ID NO: 5346;
693. CpGs whose cancer index value is determined are located within at least DMR 347 defined by SEQ ID NO: 5347, and wherein when the cancer index for the individual is about 0.043 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least eight CpG from DMR 347, and more preferably wherein the cancer index value is the mean β-value for the CpGs denoted by [[CG]] in SEQ ID NO: 5347;
694. CpGs whose cancer index value is determined are located within at least DMR 347 defined by SEQ ID NO: 5347, and wherein when the cancer index for the individual is less than about 0.043 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least eight CpG from DMR 347, and more preferably wherein the cancer index value is the mean β-value for the CpGs denoted by [[CG]] in SEQ ID NO: 5347;
695. CpGs whose cancer index value is determined are located within at least DMR 348 defined by SEQ ID NO: 5348, and wherein when the cancer index for the individual is about 0.054 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least nine CpG from DMR 348, and more preferably wherein the cancer index value is the mean β-value for the CpGs denoted by [[CG]] in SEQ ID NO: 5348;
696. CpGs whose cancer index value is determined are located within at least DMR 348 defined by SEQ ID NO: 5348, and wherein when the cancer index for the individual is less than about 0.054 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least nine CpG from DMR 348, and more preferably wherein the cancer index value is the mean β-value for the CpGs denoted by [[CG]] in SEQ ID NO: 5348;
697. CpGs whose cancer index value is determined are located within at least DMR 349 defined by SEQ ID NO: 5349, and wherein when the cancer index for the individual is about 0.054 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least nine CpG from DMR 349, and more preferably wherein the cancer index value is the mean β-value for the CpGs denoted by [[CG]] in SEQ ID NO: 5349;
698. CpGs whose cancer index value is determined are located within at least DMR 349 defined by SEQ ID NO: 5349, and wherein when the cancer index for the individual is less than about 0.054 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least nine CpG from DMR 349, and more preferably wherein the cancer index value is the mean β-value for the CpGs denoted by [[CG]] in SEQ ID NO: 5349;
699. CpGs whose cancer index value is determined are located within at least DMR 350 defined by SEQ ID NO: 5350, and wherein when the cancer index for the individual is about 0.056 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least seven CpG from DMR 350, and more preferably wherein the cancer index value is the mean β-value for the CpGs denoted by [[CG]] in SEQ ID NO: 5350;
700. CpGs whose cancer index value is determined are located within at least DMR 350 defined by SEQ ID NO: 5350, and wherein when the cancer index for the individual is less than about 0.056 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least seven CpG from DMR 350, and more preferably wherein the cancer index value is the mean β-value for the CpGs denoted by [[CG]] in SEQ ID NO: 5350;
701. CpGs whose cancer index value is determined are located within at least DMR 351 defined by SEQ ID NO: 5351, and wherein when the cancer index for the individual is about 0.031 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 351, and more preferably wherein the cancer index value is the mean β-value for the CpGs denoted by [[CG]] in SEQ ID NO: 5351;
702. CpGs whose cancer index value is determined are located within at least DMR 351 defined by SEQ ID NO: 5351, and wherein when the cancer index for the individual is less than about 0.031 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 351, and more preferably wherein the cancer index value is the mean β-value for the CpGs denoted by [[CG]] in SEQ ID NO: 5351;
703. CpGs whose cancer index value is determined are located within at least DMR 352 defined by SEQ ID NO: 5352, and wherein when the cancer index for the individual is about 0.065 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 352, and more preferably wherein the cancer index value is the mean β-value for the CpGs denoted by [[CG]] in SEQ ID NO: 5352;
704. CpGs whose cancer index value is determined are located within at least DMR 352 defined by SEQ ID NO: 5352, and wherein when the cancer index for the individual is less than about 0.065 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 352, and more preferably wherein the cancer index value is the mean β-value for the CpGs denoted by [[CG]] in SEQ ID NO: 5352;
705. CpGs whose cancer index value is determined are located within at least DMR 353 defined by SEQ ID NO: 5353, and wherein when the cancer index for the individual is about 0.049 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least six CpG from DMR 353, and more preferably wherein the cancer index value is the mean β-value for the CpGs denoted by [[CG]] in SEQ ID NO: 5353;
706. CpGs whose cancer index value is determined are located within at least DMR 353 defined by SEQ ID NO: 5353, and wherein when the cancer index for the individual is less than about 0.049 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least six CpG from DMR 353, and more preferably wherein the cancer index value is the mean β-value for the CpGs denoted by [[CG]] in SEQ ID NO: 5353;
707. CpGs whose cancer index value is determined are located within at least DMR 354 defined by SEQ ID NO: 5354, and wherein when the cancer index for the individual is about 0.052 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 354, and more preferably wherein the cancer index value is the mean β-value for the CpGs denoted by [[CG]] in SEQ ID NO: 5354;
708. CpGs whose cancer index value is determined are located within at least DMR 354 defined by SEQ ID NO: 5354, and wherein when the cancer index for the individual is less than about 0.052 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 354, and more preferably wherein the cancer index value is the mean β-value for the CpGs denoted by [[CG]] in SEQ ID NO: 5354;
709. CpGs whose cancer index value is determined are located within at least DMR 355 defined by SEQ ID NO: 5355, and wherein when the cancer index for the individual is about 0.056 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least five CpG from DMR 355, and more preferably wherein the cancer index value is the mean β-value for the CpGs denoted by [[CG]] in SEQ ID NO: 5355;
710. CpGs whose cancer index value is determined are located within at least DMR 355 defined by SEQ ID NO: 5355, and wherein when the cancer index for the individual is less than about 0.056 the individual is classified as not having CIN3 and/or cancer or as having a low risk of CIN3 and/or cancer development, and wherein the sensitivity of the assay is at least 66.00% and the specificity of the assay is at least 75.00%, preferably wherein the panel of one or more CpGs comprises at least five CpG from DMR 355, and more preferably wherein the cancer index value is the mean β-value for the CpGs denoted by [[CG]] in SEQ ID NO: 5355;
711. CpGs whose cancer index value is determined are located within at least DMR 356 defined by SEQ ID NO: 5356, and wherein when the cancer index for the individual is about 0.059 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 356, and more preferably wherein the cancer index value is the mean β-value for the CpGs denoted by [[CG]] in SEQ ID NO: 5356;
712. CpGs whose cancer index value is determined are located within at least DMR 356 defined by SEQ ID NO: 5356, and wherein when the cancer index for the individual is less than about 0.059 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 356, and more preferably wherein the cancer index value is the mean β-value for the CpGs denoted by [[CG]] in SEQ ID NO: 5356;
713. CpGs whose cancer index value is determined are located within at least DMR 357 defined by SEQ ID NO: 5357, and wherein when the cancer index for the individual is about 0.043 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least eight CpG from DMR 357, and more preferably wherein the cancer index value is the mean β-value for the CpGs denoted by [[CG]] in SEQ ID NO: 5357;
714. CpGs whose cancer index value is determined are located within at least DMR 357 defined by SEQ ID NO: 5357, and wherein when the cancer index for the individual is less than about 0.043 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least eight CpG from DMR 357, and more preferably wherein the cancer index value is the mean β-value for the CpGs denoted by [[CG]] in SEQ ID NO: 5357;
715. CpGs whose cancer index value is determined are located within at least DMR 358 defined by SEQ ID NO: 5358, and wherein when the cancer index for the individual is about 0.05 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least six CpG from DMR 358, and more preferably wherein the cancer index value is the mean β-value for the CpGs denoted by [[CG]] in SEQ ID NO: 5358;
716. CpGs whose cancer index value is determined are located within at least DMR 358 defined by SEQ ID NO: 5358, and wherein when the cancer index for the individual is less than about 0.05 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least six CpG from DMR 358, and more preferably wherein the cancer index value is the mean β-value for the CpGs denoted by [[CG]] in SEQ ID NO: 5358;
717. CpGs whose cancer index value is determined are located within at least DMR 359 defined by SEQ ID NO: 5359, and wherein when the cancer index for the individual is about 0.043 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least eight CpG from DMR 359, and more preferably wherein the cancer index value is the mean β-value for the CpGs denoted by [[CG]] in SEQ ID NO: 5359;
718. CpGs whose cancer index value is determined are located within at least DMR 359 defined by SEQ ID NO: 5359, and wherein when the cancer index for the individual is less than about 0.043 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least eight CpG from DMR 359, and more preferably wherein the cancer index value is the mean β-value for the CpGs denoted by [[CG]] in SEQ ID NO: 5359;
719. CpGs whose cancer index value is determined are located within at least DMR 360 defined by SEQ ID NO: 5360, and wherein when the cancer index for the individual is about 0.059 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least eight CpG from DMR 360, and more preferably wherein the cancer index value is the mean β-value for the CpGs denoted by [[CG]] in SEQ ID NO: 5360;
720. CpGs whose cancer index value is determined are located within at least DMR 360 defined by SEQ ID NO: 5360, and wherein when the cancer index for the individual is less than about 0.059 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least eight CpG from DMR 360, and more preferably wherein the cancer index value is the mean β-value for the CpGs denoted by [[CG]] in SEQ ID NO: 5360;
721. CpGs whose cancer index value is determined are located within at least DMR 361 defined by SEQ ID NO: 5361, and wherein when the cancer index for the individual is about 0.052 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 361, and more preferably wherein the cancer index value is the mean β-value for the CpGs denoted by [[CG]] in SEQ ID NO: 5361;
722. CpGs whose cancer index value is determined are located within at least DMR 361 defined by SEQ ID NO: 5361, and wherein when the cancer index for the individual is less than about 0.052 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 361, and more preferably wherein the cancer index value is the mean β-value for the CpGs denoted by [[CG]] in SEQ ID NO: 5361;
723. CpGs whose cancer index value is determined are located within at least DMR 362 defined by SEQ ID NO: 5362, and wherein when the cancer index for the individual is about 0.052 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 362, and more preferably wherein the cancer index value is the mean β-value for the CpGs denoted by [[CG]] in SEQ ID NO: 5362;
724. CpGs whose cancer index value is determined are located within at least DMR 362 defined by SEQ ID NO: 5362, and wherein when the cancer index for the individual is less than about 0.052 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 362, and more preferably wherein the cancer index value is the mean β-value for the CpGs denoted by [[CG]] in SEQ ID NO: 5362;
725. CpGs whose cancer index value is determined are located within at least DMR 363 defined by SEQ ID NO: 5363, and wherein when the cancer index for the individual is about 0.043 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least eight CpG from DMR 363, and more preferably wherein the cancer index value is the mean β-value for the CpGs denoted by [[CG]] in SEQ ID NO: 5363;
726. CpGs whose cancer index value is determined are located within at least DMR 363 defined by SEQ ID NO: 5363, and wherein when the cancer index for the individual is less than about 0.043 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least eight CpG from DMR 363, and more preferably wherein the cancer index value is the mean β-value for the CpGs denoted by [[CG]] in SEQ ID NO: 5363;
727. CpGs whose cancer index value is determined are located within at least DMR 364 defined by SEQ ID NO: 5364, and wherein when the cancer index for the individual is about 0.02 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least five CpG from DMR 364, and more preferably wherein the cancer index value is the mean β-value for the CpGs denoted by [[CG]] in SEQ ID NO: 5364;
728. CpGs whose cancer index value is determined are located within at least DMR 364 defined by SEQ ID NO: 5364, and wherein when the cancer index for the individual is less than about 0.02 the individual is classified as not having CIN3 and/or cancer or as having a low risk of CIN3 and/or cancer development, and wherein the sensitivity of the assay is at least 70.00% and the specificity of the assay is at least 75.00%, preferably wherein the panel of one or more CpGs comprises at least five CpG from DMR 364, and more preferably wherein the cancer index value is the mean β-value for the CpGs denoted by [[CG]] in SEQ ID NO: 5364;
729. CpGs whose cancer index value is determined are located within at least DMR 365 defined by SEQ ID NO: 5365, and wherein when the cancer index for the individual is about 0.029 or more the individual is classified as having CIN3 and/or cancer or as having a high risk of CIN3 and/or cancer development, and wherein the sensitivity of the assay is at least 68.00% and the specificity of the assay is at least 75.00%, preferably wherein the panel of one or more CpGs comprises at least six CpG from DMR 365, and more preferably wherein the cancer index value is the mean β-value for the CpGs denoted by [[CG]] in SEQ ID NO: 5365;
730. CpGs whose cancer index value is determined are located within at least DMR 365 defined by SEQ ID NO: 5365, and wherein when the cancer index for the individual is less than about 0.029 the individual is classified as not having CIN3 and/or cancer or as having a low risk of CIN3 and/or cancer development, and wherein the sensitivity of the assay is at least 68.00% and the specificity of the assay is at least 75.00%, preferably wherein the panel of one or more CpGs comprises at least six CpG from DMR 365, and more preferably wherein the cancer index value is the mean β-value for the CpGs denoted by [[CG]] in SEQ ID NO: 5365;
731. CpGs whose cancer index value is determined are located within at least DMR 366 defined by SEQ ID NO: 5366, and wherein when the cancer index for the individual is about 0.052 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 366, and more preferably wherein the cancer index value is the mean β-value for the CpGs denoted by [[CG]] in SEQ ID NO: 5366;
732. CpGs whose cancer index value is determined are located within at least DMR 366 defined by SEQ ID NO: 5366, and wherein when the cancer index for the individual is less than about 0.052 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 366, and more preferably wherein the cancer index value is the mean β-value for the CpGs denoted by [[CG]] in SEQ ID NO: 5366;
733. CpGs whose cancer index value is determined are located within at least DMR 367 defined by SEQ ID NO: 5367, and wherein when the cancer index for the individual is about 0.043 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least eight CpG from DMR 367, and more preferably wherein the cancer index value is the mean β-value for the CpGs denoted by [[CG]] in SEQ ID NO: 5367;
734. CpGs whose cancer index value is determined are located within at least DMR 367 defined by SEQ ID NO: 5367, and wherein when the cancer index for the individual is less than about 0.043 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least eight CpG from DMR 367, and more preferably wherein the cancer index value is the mean β-value for the CpGs denoted by [[CG]] in SEQ ID NO: 5367;
735. CpGs whose cancer index value is determined are located within at least DMR 368 defined by SEQ ID NO: 5368, and wherein when the cancer index for the individual is about 0.052 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 368, and more preferably wherein the cancer index value is the mean β-value for the CpGs denoted by [[CG]] in SEQ ID NO: 5368;
736. CpGs whose cancer index value is determined are located within at least DMR 368 defined by SEQ ID NO: 5368, and wherein when the cancer index for the individual is less than about 0.052 the individual is classified as not having CIN3 and/or cancer or as having a low risk of CIN3 and/or cancer development, and wherein the sensitivity of the assay is at least 69.00% and the specificity of the assay is at least 75.00%, preferably wherein the panel of one or more CpGs comprises at least nine CpG from DMR 368, and more preferably wherein the cancer index value is the mean β-value for the CpGs denoted by [[CG]] in SEQ ID NO: 5368;
737. CpGs whose cancer index value is determined are located within at least DMR 369 defined by SEQ ID NO: 5369, and wherein when the cancer index for the individual is about 0.034 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 369, and more preferably wherein the cancer index value is the mean β-value for the CpGs denoted by [[CG]] in SEQ ID NO: 5369;
738. CpGs whose cancer index value is determined are located within at least DMR 369 defined by SEQ ID NO: 5369, and wherein when the cancer index for the individual is less than about 0.034 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 369, and more preferably wherein the cancer index value is the mean β-value for the CpGs denoted by [[CG]] in SEQ ID NO: 5369;
739. CpGs whose cancer index value is determined are located within at least DMR 370 defined by SEQ ID NO: 5370, and wherein when the cancer index for the individual is about 0.034 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 370, and more preferably wherein the cancer index value is the mean β-value for the CpGs denoted by [[CG]] in SEQ ID NO: 5370;
740. CpGs whose cancer index value is determined are located within at least DMR 370 defined by SEQ ID NO: 5370, and wherein when the cancer index for the individual is less than about 0.034 the individual is classified as not having CIN3 and/or cancer or as having a low risk of CIN3 and/or cancer development, and wherein the sensitivity of the assay is at least 71.00% and the specificity of the assay is at least 75.00%, preferably wherein the panel of one or more CpGs comprises at least nine CpG from DMR 370, and more preferably wherein the cancer index value is the mean β-value for the CpGs denoted by [[CG]] in SEQ ID NO: 5370;
741. CpGs whose cancer index value is determined are located within at least DMR 371 defined by SEQ ID NO: 5371, and wherein when the cancer index for the individual is about 0.031 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 371, and more preferably wherein the cancer index value is the mean β-value for the CpGs denoted by [[CG]] in SEQ ID NO: 5371;
742. CpGs whose cancer index value is determined are located within at least DMR 371 defined by SEQ ID NO: 5371, and wherein when the cancer index for the individual is less than about 0.031 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 371, and more preferably wherein the cancer index value is the mean β-value for the CpGs denoted by [[CG]] in SEQ ID NO: 5371;
743. CpGs whose cancer index value is determined are located within at least DMR 372 defined by SEQ ID NO: 5372, and wherein when the cancer index for the individual is about 0.031 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least three CpG from DMR 372, and more preferably wherein the cancer index value is the mean β-value for the CpGs denoted by [[CG]] in SEQ ID NO: 5372;
744. CpGs whose cancer index value is determined are located within at least DMR 372 defined by SEQ ID NO: 5372, and wherein when the cancer index for the individual is less than about 0.031 the individual is classified as not having CIN3 and/or cancer or as having a low risk of CIN3 and/or cancer development, and wherein the sensitivity of the assay is at least 72.00% and the specificity of the assay is at least 75.00%, preferably wherein the panel of one or more CpGs comprises at least three CpG from DMR 372, and more preferably wherein the cancer index value is the mean β-value for the CpGs denoted by [[CG]] in SEQ ID NO: 5372;
745. CpGs whose cancer index value is determined are located within at least DMR 373 defined by SEQ ID NO: 5373, and wherein when the cancer index for the individual is about 0.072 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least three CpG from DMR 373, and more preferably wherein the cancer index value is the mean β-value for the CpGs denoted by [[CG]] in SEQ ID NO: 5373;
746. CpGs whose cancer index value is determined are located within at least DMR 373 defined by SEQ ID NO: 5373, and wherein when the cancer index for the individual is less than about 0.072 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least three CpG from DMR 373, and more preferably wherein the cancer index value is the mean β-value for the CpGs denoted by [[CG]] in SEQ ID NO: 5373;
747. CpGs whose cancer index value is determined are located within at least DMR 374 defined by SEQ ID NO: 5374, and wherein when the cancer index for the individual is about 0.058 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least two CpG from DMR 374, and more preferably wherein the cancer index value is the mean β-value for the CpGs denoted by [[CG]] in SEQ ID NO: 5374;
748. CpGs whose cancer index value is determined are located within at least DMR 374 defined by SEQ ID NO: 5374, and wherein when the cancer index for the individual is less than about 0.058 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least two CpG from DMR 374, and more preferably wherein the cancer index value is the mean β-value for the CpGs denoted by [[CG]] in SEQ ID NO: 5374;
749. CpGs whose cancer index value is determined are located within at least DMR 375 defined by SEQ ID NO: 5375, and wherein when the cancer index for the individual is about 0.061 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 375, and more preferably wherein the cancer index value is the mean β-value for the CpGs denoted by [[CG]] in SEQ ID NO: 5375;
750. CpGs whose cancer index value is determined are located within at least DMR 375 defined by SEQ ID NO: 5375, and wherein when the cancer index for the individual is less than about 0.061 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 375, and more preferably wherein the cancer index value is the mean β-value for the CpGs denoted by [[CG]] in SEQ ID NO: 5375;
751. CpGs whose cancer index value is determined are located within at least DMR 376 defined by SEQ ID NO: 5376, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 376, and more preferably wherein the cancer index value is the mean β-value for the CpGs denoted by [[CG]] in SEQ ID NO: 5376;
752. CpGs whose cancer index value is determined are located within at least DMR 376 defined by SEQ ID NO: 5376, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 376, and more preferably wherein the cancer index value is the mean β-value for the CpGs denoted by [[CG]] in SEQ ID NO: 5376;
753. CpGs whose cancer index value is determined are located within at least DMR 377 defined by SEQ ID NO: 5377, and wherein when the cancer index for the individual is about 0.051 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least five CpG from DMR 377, and more preferably wherein the cancer index value is the mean β-value for the CpGs denoted by [[CG]] in SEQ ID NO: 5377;
754. CpGs whose cancer index value is determined are located within at least DMR 377 defined by SEQ ID NO: 5377, and wherein when the cancer index for the individual is less than about 0.051 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least five CpG from DMR 377, and more preferably wherein the cancer index value is the mean β-value for the CpGs denoted by [[CG]] in SEQ ID NO: 5377;
755. CpGs whose cancer index value is determined are located within at least DMR 378 defined by SEQ ID NO: 5378, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 378, and more preferably wherein the cancer index value is the mean β-value for the CpGs denoted by [[CG]] in SEQ ID NO: 5378;
756. CpGs whose cancer index value is determined are located within at least DMR 378 defined by SEQ ID NO: 5378, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 378, and more preferably wherein the cancer index value is the mean β-value for the CpGs denoted by [[CG]] in SEQ ID NO: 5378;
757. CpGs whose cancer index value is determined are located within at least DMR 379 defined by SEQ ID NO: 5379, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 379, and more preferably wherein the cancer index value is the mean β-value for the CpGs denoted by [[CG]] in SEQ ID NO: 5379;
758. CpGs whose cancer index value is determined are located within at least DMR 379 defined by SEQ ID NO: 5379, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 379, and more preferably wherein the cancer index value is the mean β-value for the CpGs denoted by [[CG]] in SEQ ID NO: 5379;
759. CpGs whose cancer index value is determined are located within at least DMR 380 defined by SEQ ID NO: 5380, and wherein when the cancer index for the individual is about 0.028 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least four CpG from DMR 380, and more preferably wherein the cancer index value is the mean β-value for the CpGs denoted by [[CG]] in SEQ ID NO: 5380;
760. CpGs whose cancer index value is determined are located within at least DMR 380 defined by SEQ ID NO: 5380, and wherein when the cancer index for the individual is less than about 0.028 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least four CpG from DMR 380, and more preferably wherein the cancer index value is the mean β-value for the CpGs denoted by [[CG]] in SEQ ID NO: 5380;
761. CpGs whose cancer index value is determined are located within at least DMR 381 defined by SEQ ID NO: 5381, and wherein when the cancer index for the individual is about 0.028 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least four CpG from DMR 381, and more preferably wherein the cancer index value is the mean β-value for the CpGs denoted by [[CG]] in SEQ ID NO: 5381;
762. CpGs whose cancer index value is determined are located within at least DMR 381 defined by SEQ ID NO: 5381, and wherein when the cancer index for the individual is less than about 0.028 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least four CpG from DMR 381, and more preferably wherein the cancer index value is the mean β-value for the CpGs denoted by [[CG]] in SEQ ID NO: 5381;
763. CpGs whose cancer index value is determined are located within at least DMR 382 defined by SEQ ID NO: 5382, and wherein when the cancer index for the individual is about 0.028 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least four CpG from DMR 382, and more preferably wherein the cancer index value is the mean β-value for the CpGs denoted by [[CG]] in SEQ ID NO: 5382;
764. CpGs whose cancer index value is determined are located within at least DMR 382 defined by SEQ ID NO: 5382, and wherein when the cancer index for the individual is less than about 0.028 the individual is classified as not having CIN3 and/or cancer or as having a low risk of CIN3 and/or cancer development, and wherein the sensitivity of the assay is at least 64.00% and the specificity of the assay is at least 75.00%, preferably wherein the panel of one or more CpGs comprises at least four CpG from DMR 382, and more preferably wherein the cancer index value is the mean β-value for the CpGs denoted by [[CG]] in SEQ ID NO: 5382;
765. CpGs whose cancer index value is determined are located within at least DMR 383 defined by SEQ ID NO: 5383, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 383, and more preferably wherein the cancer index value is the mean β-value for the CpGs denoted by [[CG]] in SEQ ID NO: 5383;
766. CpGs whose cancer index value is determined are located within at least DMR 383 defined by SEQ ID NO: 5383, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 383, and more preferably wherein the cancer index value is the mean β-value for the CpGs denoted by [[CG]] in SEQ ID NO: 5383;
767. CpGs whose cancer index value is determined are located within at least DMR 384 defined by SEQ ID NO: 5384, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 384, and more preferably wherein the cancer index value is the mean β-value for the CpGs denoted by [[CG]] in SEQ ID NO: 5384;
768. CpGs whose cancer index value is determined are located within at least DMR 384 defined by SEQ ID NO: 5384, and wherein when the cancer index for the individual is less than about 0.048 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least nine CpG from DMR 384, and more preferably wherein the cancer index value is the mean β-value for the CpGs denoted by [[CG]] in SEQ ID NO: 5384;
769. CpGs whose cancer index value is determined are located within at least DMR 385 defined by SEQ ID NO: 5385, and wherein when the cancer index for the individual is about 0.021 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least four CpG from DMR 385, and more preferably wherein the cancer index value is the mean β-value for the CpGs denoted by [[CG]] in SEQ ID NO: 5385;
770. CpGs whose cancer index value is determined are located within at least DMR 385 defined by SEQ ID NO: 5385, and wherein when the cancer index for the individual is less than about 0.021 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least four CpG from DMR 385, and more preferably wherein the cancer index value is the mean β-value for the CpGs denoted by [[CG]] in SEQ ID NO: 5385;
771. CpGs whose cancer index value is determined are located within at least DMR 386 defined by SEQ ID NO: 5386, and wherein when the cancer index for the individual is about 0.021 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least four CpG from DMR 386, and more preferably wherein the cancer index value is the mean β-value for the CpGs denoted by [[CG]] in SEQ ID NO: 5388;
772. CpGs whose cancer index value is determined are located within at least DMR 386 defined by SEQ ID NO: 5386, and wherein when the cancer index for the individual is less than about 0.021 the individual is classified as not having CIN3 and/or cancer or as having a low risk of CIN3 and/or cancer development, and wherein the sensitivity of the assay is at least 67.00% and the specificity of the assay is at least 75.00%, preferably wherein the panel of one or more CpGs comprises at least four CpG from DMR 386, and more preferably wherein the cancer index value is the mean β-value for the CpGs denoted by [[CG]] in SEQ ID NO: 5388;
773. CpGs whose cancer index value is determined are located within at least DMR 387 defined by SEQ ID NO: 5387, and wherein when the cancer index for the individual is about 0.047 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 387, and more preferably wherein the cancer index value is the mean β-value for the CpGs denoted by [[CG]] in SEQ ID NO: 5387;
774. CpGs whose cancer index value is determined are located within at least DMR 387 defined by SEQ ID NO: 5387, and wherein when the cancer index for the individual is less than about 0.047 or more the individual is classified as not having cancer or as having a low risk of cancer development, and wherein the sensitivity of the assay is at least 74.00% and the specificity of the assay is at least 75.00%, preferably wherein the panel of one or more CpGs comprises at least four CpG from DMR 387, and more preferably wherein the cancer index value is the mean β-value for the CpGs denoted by [[CG]] in SEQ ID NO: 5387;
775. CpGs whose cancer index value is determined are located within at least DMR 388 defined by SEQ ID NO: 5388, and wherein when the cancer index for the individual is about 0.047 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least four CpG from DMR 388, and more preferably wherein the cancer index value is the mean β-value for the CpGs denoted by [[CG]] in SEQ ID NO: 5388;
776. CpGs whose cancer index value is determined are located within at least DMR 388 defined by SEQ ID NO: 5388, and wherein when the cancer index for the individual is less than about 0.047 the individual is classified as not having CIN3 and/or cancer or as having a low risk of CIN3 and/or cancer development, and wherein the sensitivity of the assay is at least 74.00% and the specificity of the assay is at least 75.00%, preferably wherein the panel of one or more CpGs comprises at least four CpG from DMR 388, and more preferably wherein the cancer index value is the mean β-value for the CpGs denoted by [[CG]] in SEQ ID NO: 5388;
777. CpGs whose cancer index value is determined are located within at least DMR 389 defined by SEQ ID NO: 5389, and wherein when the cancer index for the individual is about 0.042 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least nine CpG from DMR 389, and more preferably wherein the cancer index value is the mean β-value for the CpGs denoted by [[CG]] in SEQ ID NO: 5389;
778. CpGs whose cancer index value is determined are located within at least DMR 389 defined by SEQ ID NO: 5389, and wherein when the cancer index for the individual is less than about 0.042 the individual is classified as not having CIN3 and/or cancer or as having a low risk of CIN3 and/or cancer development, and wherein the sensitivity of the assay is at least 66.00% and the specificity of the assay is at least 75.00%, preferably wherein the panel of one or more CpGs comprises at least nine CpG from DMR 389, and more preferably wherein the cancer index value is the mean β-value for the CpGs denoted by [[CG]] in SEQ ID NO: 5389;
779. CpGs whose cancer index value is determined are located within at least DMR 390 defined by SEQ ID NO: 5390, and wherein when the cancer index for the individual is about 0.048 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 390, and more preferably wherein the cancer index value is the mean β-value for the CpGs denoted by [[CG]] in SEQ ID NO: 5390;
780. CpGs whose cancer index value is determined are located within at least DMR 390 defined by SEQ ID NO: 5390, and wherein when the cancer index for the individual is less than about 0.048 or more the individual is classified as not having cancer or as having a low risk of cancer development, and wherein the sensitivity of the assay is at least 67.00% and the specificity of the assay is at least 75.00%, preferably wherein the panel of one or more CpGs comprises at least nine CpG from DMR 390, and more preferably wherein the cancer index value is the mean β-value for the CpGs denoted by [[CG]] in SEQ ID NO: 5390;
781. CpGs whose cancer index value is determined are located within at least DMR 391 defined by SEQ ID NO: 5391, and wherein when the cancer index for the individual is about 0.048 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 391, and more preferably wherein the cancer index value is the mean β-value for the CpGs denoted by [[CG]] in SEQ ID NO: 5391;
782. CpGs whose cancer index value is determined are located within at least DMR 391 defined by SEQ ID NO: 5391, and wherein when the cancer index for the individual is less than about 0.048 or more the individual is classified as not having cancer or as having a low risk of cancer development, and wherein the sensitivity of the assay is at least 80.00% and the specificity of the assay is at least 75.00%, preferably wherein the panel of one or more CpGs comprises at least nine CpG from DMR 391, and more preferably wherein the cancer index value is the mean β-value for the CpGs denoted by [[CG]] in SEQ ID NO: 5391;
783. CpGs whose cancer index value is determined are located within at least DMR 392 defined by SEQ ID NO: 5392, and wherein when the cancer index for the individual is about 0.027 or more the individual is classified as having CIN3 and/or cancer or as having a high risk of CIN3 and/or cancer development, and wherein the sensitivity of the assay is at least 83.00% and the specificity of the assay is at least 75.00%, preferably wherein the panel of one or more CpGs comprises at least nine CpG from DMR 392, and more preferably wherein the cancer index value is the mean β-value for the CpGs denoted by [[CG]] in SEQ ID NO: 5392;
784. CpGs whose cancer index value is determined are located within at least DMR 392 defined by SEQ ID NO: 5392, and wherein when the cancer index for the individual is less than about 0.027 or more the individual is classified as not having cancer or as having a low risk of cancer development, and wherein the sensitivity of the assay is at least 83.00% and the specificity of the assay is at least 75.00%, preferably wherein the panel of one or more CpGs comprises at least nine CpG from DMR 392, and more preferably wherein the cancer index value is the mean β-value for the CpGs denoted by [[CG]] in SEQ ID NO: 5392;
785. CpGs whose cancer index value is determined are located within at least DMR 393 defined by SEQ ID NO: 5393, and wherein when the cancer index for the individual is about 0.038 or more the individual is classified as having CIN3 and/or cancer or as having a high risk of CIN3 and/or cancer development, and wherein the sensitivity of the assay is at least 80.00% and the specificity of the assay is at least 75.00%, preferably wherein the panel of one or more CpGs comprises at least nine CpG from DMR 393, and more preferably wherein the cancer index value is the mean β-value for the CpGs denoted by [[CG]] in SEQ ID NO: 5393;
786. CpGs whose cancer index value is determined are located within at least DMR 393 defined by SEQ ID NO: 5393, and wherein when the cancer index for the individual is less than about 0.038 or more the individual is classified as not having cancer or as having a low risk of cancer development, and wherein the sensitivity of the assay is at least 80.00% and the specificity of the assay is at least 75.00%, preferably wherein the panel of one or more CpGs comprises at least nine CpG from DMR 393, and more preferably wherein the cancer index value is the mean β-value for the CpGs denoted by [[CG]] in SEQ ID NO: 5393;
787. CpGs whose cancer index value is determined are located within at least DMR 394 defined by SEQ ID NO: 5394, and wherein when the cancer index for the individual is about 0.036 or more the individual is classified as having CIN3 and/or cancer or as having a high risk of CIN3 and/or cancer development, and wherein the sensitivity of the assay is at least 82.00% and the specificity of the assay is at least 75.00%, preferably wherein the panel of one or more CpGs comprises at least nine CpG from DMR 394, and more preferably wherein the cancer index value is the mean β-value for the CpGs denoted by [[CG]] in SEQ ID NO: 5394;
788. CpGs whose cancer index value is determined are located within at least DMR 394 defined by SEQ ID NO: 5394, and wherein when the cancer index for the individual is less than about 0.036 or more the individual is classified as not having cancer or as having a low risk of cancer development, and wherein the sensitivity of the assay is at least 82.00% and the specificity of the assay is at least 75.00%, preferably wherein the panel of one or more CpGs comprises at least nine CpG from DMR 394, and more preferably wherein the cancer index value is the mean β-value for the CpGs denoted by [[CG]] in SEQ ID NO: 5394;
789. CpGs whose cancer index value is determined are located within at least DMR 395 defined by SEQ ID NO: 5395, and wherein when the cancer index for the individual is about 0.030 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least nine CpG from DMR 395, and more preferably wherein the cancer index value is the mean β-value for the CpGs denoted by [[CG]] in SEQ ID NO: 5395;
790. CpGs whose cancer index value is determined are located within at least DMR 395 defined by SEQ ID NO: 5395, and wherein when the cancer index for the individual is less than about 0.030 or more the individual is classified as not having cancer or as having a low risk of cancer development, and wherein the sensitivity of the assay is at least 76.00% and the specificity of the assay is at least 75.00%, preferably wherein the panel of one or more CpGs comprises at least nine CpG from DMR 395, and more preferably wherein the cancer index value is the mean β-value for the CpGs denoted by [[CG]] in SEQ ID NO: 5395;
791. CpGs whose cancer index value is determined are located within at least DMR 396 defined by SEQ ID NO: 5396, and wherein when the cancer index for the individual is about 0.042 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least nine CpG from DMR 396, and more preferably wherein the cancer index value is the mean β-value for the CpGs denoted by [[CG]] in SEQ ID NO: 5396;
792. CpGs whose cancer index value is determined are located within at least DMR 396 defined by SEQ ID NO: 5396, and wherein when the cancer index for the individual is less than about 0.042 or more the individual is classified as not having cancer or as having a low risk of cancer development, and wherein the sensitivity of the assay is at least 79.00% and the specificity of the assay is at least 75.00%, preferably wherein the panel of one or more CpGs comprises at least nine CpG from DMR 396, and more preferably wherein the cancer index value is the mean β-value for the CpGs denoted by [[CG]] in SEQ ID NO: 5396;
793. CpGs whose cancer index value is determined are located within at least DMR 397 defined by SEQ ID NO: 5397, and wherein when the cancer index for the individual is about 0.060 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least nine CpG from DMR 397, and more preferably wherein the cancer index value is the mean β-value for the CpGs denoted by [[CG]] in SEQ ID NO: 5397;
794. CpGs whose cancer index value is determined are located within at least DMR 397 defined by SEQ ID NO: 5397, and wherein when the cancer index for the individual is less than about 0.060 or more the individual is classified as not having cancer or as having a low risk of cancer development, and wherein the sensitivity of the assay is at least 77.00% and the specificity of the assay is at least 75.00%, preferably wherein the panel of one or more CpGs comprises at least nine CpG from DMR 397, and more preferably wherein the cancer index value is the mean β-value for the CpGs denoted by [[CG]] in SEQ ID NO: 5397;
795. CpGs whose cancer index value is determined are located within at least DMR 398 defined by SEQ ID NO: 5398, and wherein when the cancer index for the individual is about 0.042 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 398, and more preferably wherein the cancer index value is the mean β-value for the CpGs denoted by [[CG]] in SEQ ID NO: 5398;
796. CpGs whose cancer index value is determined are located within at least DMR 398 defined by SEQ ID NO: 5398, and wherein when the cancer index for the individual is less than about 0.042 or more the individual is classified as not having cancer or as having a low risk of cancer development, and wherein the sensitivity of the assay is at least 71.00% and the specificity of the assay is at least 75.00%, preferably wherein the panel of one or more CpGs comprises at least nine CpG from DMR 398, and more preferably wherein the cancer index value is the mean β-value for the CpGs denoted by [[CG]] in SEQ ID NO: 5398;
797. CpGs whose cancer index value is determined are located within at least DMR 399 defined by SEQ ID NO: 5399, and wherein when the cancer index for the individual is about 0.050 or more the individual is classified as having CIN3 and/or cancer or as having a high risk of CIN3 and/or cancer development, and wherein the sensitivity of the assay is at least 79.00% and the specificity of the assay is at least 75.00%, preferably wherein the panel of one or more CpGs comprises at least nine CpG from DMR 399, and more preferably wherein the cancer index value is the mean β-value for the CpGs denoted by [[CG]] in SEQ ID NO: 5399;
798. CpGs whose cancer index value is determined are located within at least DMR 399 defined by SEQ ID NO: 5399, and wherein when the cancer index for the individual is less than about 0.042 or more the individual is classified as not having cancer or as having a low risk of cancer development, and wherein the sensitivity of the assay is at least 79.00% and the specificity of the assay is at least 75.00%, preferably wherein the panel of one or more CpGs comprises at least nine CpG from DMR 399, and more preferably wherein the cancer index value is the mean β-value for the CpGs denoted by [[CG]] in SEQ ID NO: 5399;
799. CpGs whose cancer index value is determined are located within at least DMR 400 defined by SEQ ID NO: 5400, and wherein when the cancer index for the individual is about 0.032 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least nine CpG from DMR 400, and more preferably wherein the cancer index value is the mean β-value for the CpGs denoted by [[CG]] in SEQ ID NO: 5400;
800. CpGs whose cancer index value is determined are located within at least DMR 400 defined by SEQ ID NO: 5400, and wherein when the cancer index for the individual is less than about 0.032 or more the individual is classified as not having cancer or as having a low risk of cancer development, and wherein the sensitivity of the assay is at least 77.00% and the specificity of the assay is at least 75.00%, preferably wherein the panel of one or more CpGs comprises at least nine CpG from DMR 400, and more preferably wherein the cancer index value is the mean β-value for the CpGs denoted by [[CG]] in SEQ ID NO: 5400;
801. CpGs whose cancer index value is determined are located within at least DMR 401 defined by SEQ ID NO: 5401, and wherein when the cancer index for the individual is about 0.088 or more the individual is classified as having CIN3 and/or cancer or as having a high risk of CIN3 and/or cancer development, and wherein the sensitivity of the assay is at least 77.00% and the specificity of the assay is at least 75.00%, preferably wherein the panel of one or more CpGs comprises at least nine CpG from DMR 401, and more preferably wherein the cancer index value is the mean β-value for the CpGs denoted by [[CG]] in SEQ ID NO: 5401;
802. CpGs whose cancer index value is determined are located within at least DMR 401 defined by SEQ ID NO: 5401, and wherein when the cancer index for the individual is less than about 0.088 or more the individual is classified as not having cancer or as having a low risk of cancer development, and wherein the sensitivity of the assay is at least 77.00% and the specificity of the assay is at least 75.00%, preferably wherein the panel of one or more CpGs comprises at least nine CpG from DMR 401, and more preferably wherein the cancer index value is the mean β-value for the CpGs denoted by [[CG]] in SEQ ID NO: 5401;
803. CpGs whose cancer index value is determined are located within at least DMR 402 defined by SEQ ID NO: 5402, and wherein when the cancer index for the individual is about 0.084 or more the individual is classified as having CIN3 and/or cancer or as having a high risk of CIN3 and/or cancer development, and wherein the sensitivity of the assay is at least 76.00% and the specificity of the assay is at least 75.00%, preferably wherein the panel of one or more CpGs comprises at least nine CpG from DMR 402, and more preferably wherein the cancer index value is the mean β-value for the CpGs denoted by [[CG]] in SEQ ID NO: 5402;
804. CpGs whose cancer index value is determined are located within at least DMR 402 defined by SEQ ID NO: 5402, and wherein when the cancer index for the individual is less than about 0.084 or more the individual is classified as not having cancer or as having a low risk of cancer development, and wherein the sensitivity of the assay is at least 76.00% and the specificity of the assay is at least 75.00%, preferably wherein the panel of one or more CpGs comprises at least nine CpG from DMR 402, and more preferably wherein the cancer index value is the mean β-value for the CpGs denoted by [[CG]] in SEQ ID NO: 5402;
805. CpGs whose cancer index value is determined are located within at least DMR 403 defined by SEQ ID NO: 5403, and wherein when the cancer index for the individual is about 0.058 or more the individual is classified as having CIN3 and/or cancer or as having a high risk of CIN3 and/or cancer development, and wherein the sensitivity of the assay is at least 72.00% and the specificity of the assay is at least 75.00%, preferably wherein the panel of one or more CpGs comprises at least nine CpG from DMR 403, and more preferably wherein the cancer index value is the mean β-value for the CpGs denoted by [[CG]] in SEQ ID NO: 5403;
806. CpGs whose cancer index value is determined are located within at least DMR 403 defined by SEQ ID NO: 5403, and wherein when the cancer index for the individual is less than about 0.058 or more the individual is classified as not having cancer or as having a low risk of cancer development, and wherein the sensitivity of the assay is at least 72.00% and the specificity of the assay is at least 75.00%, preferably wherein the panel of one or more CpGs comprises at least nine CpG from DMR 403, and more preferably wherein the cancer index value is the mean β-value for the CpGs denoted by [[CG]] in SEQ ID NO: 5403;
807. CpGs whose cancer index value is determined are located within at least DMR 404 defined by SEQ ID NO: 5404, and wherein when the cancer index for the individual is about 0.023 or more the individual is classified as having CIN3 and/or cancer or as having a high risk of CIN3 and/or cancer development, and wherein the sensitivity of the assay is at least 74.00% and the specificity of the assay is at least 75.00%, preferably wherein the panel of one or more CpGs comprises at least nine CpG from DMR 404, and more preferably wherein the cancer index value is the mean β-value for the CpGs denoted by [[CG]] in SEQ ID NO: 5404;
808. CpGs whose cancer index value is determined are located within at least DMR 404 defined by SEQ ID NO: 5404, and wherein when the cancer index for the individual is less than about 0.023 or more the individual is classified as not having cancer or as having a low risk of cancer development, and wherein the sensitivity of the assay is at least 74.00% and the specificity of the assay is at least 75.00%, preferably wherein the panel of one or more CpGs comprises at least nine CpG from DMR 404, and more preferably wherein the cancer index value is the mean β-value for the CpGs denoted by [[CG]] in SEQ ID NO: 5404;
809. CpGs whose cancer index value is determined are located within at least DMR 405 defined by SEQ ID NO: 5405, and wherein when the cancer index for the individual is about 0.187 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 405, and more preferably wherein the cancer index value is the mean β-value for the CpGs denoted by [[CG]] in SEQ ID NO: 5405;
810. CpGs whose cancer index value is determined are located within at least DMR 405 defined by SEQ ID NO: 5405, and wherein when the cancer index for the individual is less than about 0.187 or more the individual is classified as not having cancer or as having a low risk of cancer development, and wherein the sensitivity of the assay is at least 67.00% and the specificity of the assay is at least 75.00%, preferably wherein the panel of one or more CpGs comprises at least nine CpG from DMR 405, and more preferably wherein the cancer index value is the mean β-value for the CpGs denoted by [[CG]] in SEQ ID NO: 5405;
811. CpGs whose cancer index value is determined are located within at least DMR 406 defined by SEQ ID NO: 5406, and wherein when the cancer index for the individual is about 0.091 or more the individual is classified as having CIN3 and/or cancer or as having a high risk of CIN3 and/or cancer development, and wherein the sensitivity of the assay is at least 69.00% and the specificity of the assay is at least 75.00%, preferably wherein the panel of one or more CpGs comprises at least nine CpG from DMR 406, and more preferably wherein the cancer index value is the mean β-value for the CpGs denoted by [[CG]] in SEQ ID NO: 5406;
812. CpGs whose cancer index value is determined are located within at least DMR 406 defined by SEQ ID NO: 5406, and wherein when the cancer index for the individual is less than about 0.091 or more the individual is classified as not having cancer or as having a low risk of cancer development, and wherein the sensitivity of the assay is at least 69.00% and the specificity of the assay is at least 75.00%, preferably wherein the panel of one or more CpGs comprises at least nine CpG from DMR 406, and more preferably wherein the cancer index value is the mean β-value for the CpGs denoted by [[CG]] in SEQ ID NO: 5406;
813. CpGs whose cancer index value is determined are located within at least DMR 407 defined by SEQ ID NO: 5407, and wherein when the cancer index for the individual is about 0.032 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least nine CpG from DMR 407, and more preferably wherein the cancer index value is the mean β-value for the CpGs denoted by [[CG]] in SEQ ID NO: 5407;
814. CpGs whose cancer index value is determined are located within at least DMR 407 defined by SEQ ID NO: 5407, and wherein when the cancer index for the individual is less than about 0.032 or more the individual is classified as not having cancer or as having a low risk of cancer development, and wherein the sensitivity of the assay is at least 64.00% and the specificity of the assay is at least 75.00%, preferably wherein the panel of one or more CpGs comprises at least nine CpG from DMR 407, and more preferably wherein the cancer index value is the mean β-value for the CpGs denoted by [[CG]] in SEQ ID NO: 5407;
815. CpGs whose cancer index value is determined are located within at least DMR 408 defined by SEQ ID NO: 5408, and wherein when the cancer index for the individual is about 0.054 or more the individual is classified as having CIN3 and/or cancer or as having a high risk of CIN3 and/or cancer development, and wherein the sensitivity of the assay is at least 70.00% and the specificity of the assay is at least 75.00%, preferably wherein the panel of one or more CpGs comprises at least nine CpG from DMR 408, and more preferably wherein the cancer index value is the mean β-value for the CpGs denoted by [[CG]] in SEQ ID NO: 5408;
816. CpGs whose cancer index value is determined are located within at least DMR 408 defined by SEQ ID NO: 5408, and wherein when the cancer index for the individual is less than about 0.054 or more the individual is classified as not having cancer or as having a low risk of cancer development, and wherein the sensitivity of the assay is at least 70.00% and the specificity of the assay is at least 75.00%, preferably wherein the panel of one or more CpGs comprises at least nine CpG from DMR 408, and more preferably wherein the cancer index value is the mean β-value for the CpGs denoted by [[CG]] in SEQ ID NO: 5408;
817. CpGs whose cancer index value is determined are located within at least DMR 409 defined by SEQ ID NO: 5409, and wherein when the cancer index for the individual is about 0.034 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 409, and more preferably wherein the cancer index value is the mean β-value for the CpGs denoted by [[CG]] in SEQ ID NO: 5409;
818. CpGs whose cancer index value is determined are located within at least DMR 409 defined by SEQ ID NO: 5409, and wherein when the cancer index for the individual is less than about 0.034 or more the individual is classified as not having cancer or as having a low risk of cancer development, and wherein the sensitivity of the assay is at least 67.00% and the specificity of the assay is at least 75.00%, preferably wherein the panel of one or more CpGs comprises at least nine CpG from DMR 409, and more preferably wherein the cancer index value is the mean β-value for the CpGs denoted by [[CG]] in SEQ ID NO: 5409;
819. CpGs whose cancer index value is determined are located within at least DMR 410 defined by SEQ ID NO: 5410, and wherein when the cancer index for the individual is about 0.062 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least nine CpG from DMR 410, and more preferably wherein the cancer index value is the mean β-value for the CpGs denoted by [[CG]] in SEQ ID NO: 5410;
820. CpGs whose cancer index value is determined are located within at least DMR 410 defined by SEQ ID NO: 5410, and wherein when the cancer index for the individual is less than about 0.062 or more the individual is classified as not having cancer or as having a low risk of cancer development, and wherein the sensitivity of the assay is at least 64.00% and the specificity of the assay is at least 75.00%, preferably wherein the panel of one or more CpGs comprises at least nine CpG from DMR 410, and more preferably wherein the cancer index value is the mean β-value for the CpGs denoted by [[CG]] in SEQ ID NO: 5410;
821. CpGs whose cancer index value is determined are located within at least DMR 411 defined by SEQ ID NO: 5411, and wherein when the cancer index for the individual is about 0.082 or more the individual is classified as having CIN3 and/or cancer or as having a high risk of CIN3 and/or cancer development, and wherein the sensitivity of the assay is at least 67.00% and the specificity of the assay is at least 75.00%, preferably wherein the panel of one or more CpGs comprises at least nine CpG from DMR 411, and more preferably wherein the cancer index value is the mean β-value for the CpGs denoted by [[CG]] in SEQ ID NO: 5411;
822. CpGs whose cancer index value is determined are located within at least DMR 411 defined by SEQ ID NO: 5408, and wherein when the cancer index for the individual is less than about 0.082 or more the individual is classified as not having cancer or as having a low risk of cancer development, and wherein the sensitivity of the assay is at least 67.00% and the specificity of the assay is at least 75.00%, preferably wherein the panel of one or more CpGs comprises at least nine CpG from DMR 411, and more preferably wherein the cancer index value is the mean β-value for the CpGs denoted by [[CG]] in SEQ ID NO: 5411;
823. CpGs whose cancer index value is determined are located within at least DMR 412 defined by SEQ ID NO: 5412, and wherein when the cancer index for the individual is about 0.062 or more the individual is classified as having CIN3 and/or cancer or as having a high risk of CIN3 and/or cancer development, and wherein the sensitivity of the assay is at least 71.00% and the specificity of the assay is at least 75.00%, preferably wherein the panel of one or more CpGs comprises at least nine CpG from DMR 412, and more preferably wherein the cancer index value is the mean β-value for the CpGs denoted by [[CG]] in SEQ ID NO: 5412;
824. CpGs whose cancer index value is determined are located within at least DMR 412 defined by SEQ ID NO: 5412, and wherein when the cancer index for the individual is less than about 0.062 or more the individual is classified as not having cancer or as having a low risk of cancer development, and wherein the sensitivity of the assay is at least 71.00% and the specificity of the assay is at least 75.00%, preferably wherein the panel of one or more CpGs comprises at least nine CpG from DMR 412, and more preferably wherein the cancer index value is the mean β-value for the CpGs denoted by [[CG]] in SEQ ID NO: 5412;
825. CpGs whose cancer index value is determined are located within at least DMR 413 defined by SEQ ID NO: 5413, and wherein when the cancer index for the individual is about 0.072 or more the individual is classified as having CIN3 and/or cancer or as having a high risk of CIN3 and/or cancer development, and wherein the sensitivity of the assay is at least 63.00% and the specificity of the assay is at least 75.00%, preferably wherein the panel of one or more CpGs comprises at least nine CpG from DMR 413, and more preferably wherein the cancer index value is the mean β-value for the CpGs denoted by [[CG]] in SEQ ID NO: 5413;
826. CpGs whose cancer index value is determined are located within at least DMR 413 defined by SEQ ID NO: 5413, and wherein when the cancer index for the individual is less than about 0.072 or more the individual is classified as not having cancer or as having a low risk of cancer development, and wherein the sensitivity of the assay is at least 63.00% and the specificity of the assay is at least 75.00%, preferably wherein the panel of one or more CpGs comprises at least nine CpG from DMR 413, and more preferably wherein the cancer index value is the mean β-value for the CpGs denoted by [[CG]] in SEQ ID NO: 5413;
827. CpGs whose cancer index value is determined are located within at least DMR 414 defined by SEQ ID NO: 5414, and wherein when the cancer index for the individual is about 0.043 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least nine CpG from DMR 414, and more preferably wherein the cancer index value is the mean β-value for the CpGs denoted by [[CG]] in SEQ ID NO: 5414;
828. CpGs whose cancer index value is determined are located within at least DMR 414 defined by SEQ ID NO: 5414, and wherein when the cancer index for the individual is less than about 0.043 or more the individual is classified as not having cancer or as having a low risk of cancer development, and wherein the sensitivity of the assay is at least 66.00% and the specificity of the assay is at least 75.00%, preferably wherein the panel of one or more CpGs comprises at least nine CpG from DMR 414, and more preferably wherein the cancer index value is the mean β-value for the CpGs denoted by [[CG]] in SEQ ID NO: 5414;
829. CpGs whose cancer index value is determined are located within at least DMR 415 defined by SEQ ID NO: 5415, and wherein when the cancer index for the individual is about 0.203 or more the individual is classified as having CIN3 and/or cancer or as having a high risk of CIN3 and/or cancer development, and wherein the sensitivity of the assay is at least 66.00% and the specificity of the assay is at least 75.00%, preferably wherein the panel of one or more CpGs comprises at least nine CpG from DMR 415, and more preferably wherein the cancer index value is the mean β-value for the CpGs denoted by [[CG]] in SEQ ID NO: 5415;
830. CpGs whose cancer index value is determined are located within at least DMR 415 defined by SEQ ID NO: 5415, and wherein when the cancer index for the individual is less than about 0.203 or more the individual is classified as not having cancer or as having a low risk of cancer development, and wherein the sensitivity of the assay is at least 66.00% and the specificity of the assay is at least 75.00%, preferably wherein the panel of one or more CpGs comprises at least nine CpG from DMR 415, and more preferably wherein the cancer index value is the mean β-value for the CpGs denoted by [[CG]] in SEQ ID NO: 5415;
831. CpGs whose cancer index value is determined are located within at least DMR 416 defined by SEQ ID NO: 5416, and wherein when the cancer index for the individual is about 0.098 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least nine CpG from DMR 416, and more preferably wherein the cancer index value is the mean β-value for the CpGs denoted by [[CG]] in SEQ ID NO: 5416;
832. CpGs whose cancer index value is determined are located within at least DMR 416 defined by SEQ ID NO: 5416, and wherein when the cancer index for the individual is less than about 0.098 or more the individual is classified as not having cancer or as having a low risk of cancer development, and wherein the sensitivity of the assay is at least 64.00% and the specificity of the assay is at least 75.00%, preferably wherein the panel of one or more CpGs comprises at least nine CpG from DMR 416, and more preferably wherein the cancer index value is the mean β-value for the CpGs denoted by [[CG]] in SEQ ID NO: 5416;
833. CpGs whose cancer index value is determined are located within at least DMR 417 defined by SEQ ID NO: 5417, and wherein when the cancer index for the individual is about 0.057 or more the individual is classified as having CIN3 and/or cancer or as having a high risk of CIN3 and/or cancer development, and wherein the sensitivity of the assay is at least 64.00% and the specificity of the assay is at least 75.00%, preferably wherein the panel of one or more CpGs comprises at least nine CpG from DMR 417, and more preferably wherein the cancer index value is the mean β-value for the CpGs denoted by [[CG]] in SEQ ID NO: 5417;
834. CpGs whose cancer index value is determined are located within at least DMR 417 defined by SEQ ID NO: 5417, and wherein when the cancer index for the individual is less than about 0.057 or more the individual is classified as not having cancer or as having a low risk of cancer development, and wherein the sensitivity of the assay is at least 64.00% and the specificity of the assay is at least 75.00%, preferably wherein the panel of one or more CpGs comprises at least nine CpG from DMR 417, and more preferably wherein the cancer index value is the mean β-value for the CpGs denoted by [[CG]] in SEQ ID NO: 5417;
835. CpGs whose cancer index value is determined are located within at least DMR 418 defined by SEQ ID NO: 5418, and wherein when the cancer index for the individual is about 0.111 or more the individual is classified as having CIN3 and/or cancer or as having a high risk of CIN3 and/or cancer development, and wherein the sensitivity of the assay is at least 62.00% and the specificity of the assay is at least 75.00%, preferably wherein the panel of one or more CpGs comprises at least nine CpG from DMR 418, and more preferably wherein the cancer index value is the mean β-value for the CpGs denoted by [[CG]] in SEQ ID NO: 5418;
836. CpGs whose cancer index value is determined are located within at least DMR 418 defined by SEQ ID NO: 5418, and wherein when the cancer index for the individual is less than about 0.111 or more the individual is classified as not having cancer or as having a low risk of cancer development, and wherein the sensitivity of the assay is at least 62.00% and the specificity of the assay is at least 75.00%, preferably wherein the panel of one or more CpGs comprises at least nine CpG from DMR 418, and more preferably wherein the cancer index value is the mean β-value for the CpGs denoted by [[CG]] in SEQ ID NO: 5418.

In any of the described assays, the methylation status of the one or more CpGs in the panel is preferably determined by a β-value analysis, and the assay is for assessing the presence, absence or development of CIN3 and/or cancer, preferably the cancer is cervical cancer or endometrial cancer. Most preferably, the cancer is cervical cancer.

The ROC data set out in Table 7 corresponding to each of SEQ ID NOs: 5001 to 5418 are derived by determining a cancer index value from a panel of CpGs in each instance whereby the panel comprises the CpGs denoted by [[CG]].

TABLE 7

| seq_ID | chr | start | stop | mean_CIN3+_cases | mean_controls | cutoff | specificity | sensitivity | auc |
|---|---|---|---|---|---|---|---|---|---|
| 5001 | chr15 | 26108091 | 26108691 | 0.078 | 0.022 | 0.023 | 0.75 | 0.85 | 0.894 |
| 5002 | chr15 | 26108101 | 26108701 | 0.078 | 0.022 | 0.023 | 0.75 | 0.85 | 0.894 |
| 5003 | chr15 | 26108110 | 26108710 | 0.078 | 0.022 | 0.023 | 0.75 | 0.85 | 0.894 |
| 5004 | chr15 | 26108099 | 26108699 | 0.078 | 0.022 | 0.023 | 0.75 | 0.85 | 0.894 |
| 5005 | chr15 | 26108112 | 26108712 | 0.078 | 0.022 | 0.023 | 0.75 | 0.85 | 0.894 |
| 5006 | chr19 | 38182755 | 38183355 | 0.08 | 0.026 | 0.027 | 0.75 | 0.83 | 0.876 |
| 5007 | chr1 | 76539922 | 76540522 | 0.079 | 0.026 | 0.027 | 0.75 | 0.8 | 0.874 |
| 5008 | chr1 | 76539934 | 76540534 | 0.079 | 0.026 | 0.027 | 0.75 | 0.8 | 0.874 |
| 5009 | chr1 | 76539929 | 76540529 | 0.079 | 0.026 | 0.027 | 0.75 | 0.8 | 0.874 |
| 5010 | chr1 | 76539936 | 76540536 | 0.079 | 0.026 | 0.027 | 0.75 | 0.8 | 0.874 |
| 5011 | chr1 | 76539926 | 76540526 | 0.079 | 0.026 | 0.027 | 0.75 | 0.8 | 0.874 |
| 5012 | chr1 | 76539868 | 76540468 | 0.079 | 0.026 | 0.027 | 0.75 | 0.8 | 0.874 |
| 5013 | chr1 | 76539848 | 76540448 | 0.079 | 0.026 | 0.027 | 0.75 | 0.8 | 0.874 |
| 5014 | chr18 | 5629383 | 5629983 | 0.083 | 0.019 | 0.02 | 0.75 | 0.8 | 0.87 |
| 5015 | chr8 | 72755758 | 72756358 | 0.089 | 0.025 | 0.026 | 0.75 | 0.83 | 0.869 |
| 5016 | chr8 | 72755855 | 72756455 | 0.089 | 0.025 | 0.026 | 0.75 | 0.83 | 0.869 |
| 5017 | chr8 | 72755853 | 72756453 | 0.089 | 0.025 | 0.026 | 0.75 | 0.83 | 0.869 |
| 5018 | chr15 | 26108312 | 26108912 | 0.068 | 0.023 | 0.025 | 0.75 | 0.78 | 0.868 |
| 5019 | chr15 | 26108342 | 26108942 | 0.068 | 0.023 | 0.025 | 0.75 | 0.78 | 0.868 |
| 5020 | chr1 | 76540138 | 76540738 | 0.081 | 0.027 | 0.028 | 0.75 | 0.8 | 0.866 |
| 5021 | chr18 | 5543501 | 5544101 | 0.088 | 0.029 | 0.03 | 0.75 | 0.8 | 0.863 |
| 5022 | chr18 | 5629590 | 5630190 | 0.079 | 0.018 | 0.019 | 0.75 | 0.82 | 0.863 |
| 5023 | chr8 | 72755571 | 72756171 | 0.077 | 0.027 | 0.029 | 0.75 | 0.82 | 0.862 |
| 5024 | chr1 | 2.14E+08 | 2.14E+08 | 0.082 | 0.035 | 0.039 | 0.75 | 0.78 | 0.861 |
| 5025 | chr1 | 75601867 | 75602467 | 0.126 | 0.046 | 0.048 | 0.75 | 0.8 | 0.86 |
| 5026 | chr1 | 75602151 | 75602751 | 0.126 | 0.046 | 0.048 | 0.75 | 0.8 | 0.86 |
| 5027 | chr8 | 72756007 | 72756607 | 0.088 | 0.022 | 0.022 | 0.75 | 0.79 | 0.86 |
| 5028 | chr1 | 75602112 | 75602712 | 0.126 | 0.046 | 0.048 | 0.75 | 0.8 | 0.86 |
| 5029 | chr8 | 72756041 | 72756641 | 0.088 | 0.022 | 0.022 | 0.75 | 0.79 | 0.86 |
| 5030 | chr12 | 1.29E+08 | 1.29E+08 | 0.092 | 0.031 | 0.034 | 0.75 | 0.76 | 0.859 |
| 5031 | chr19 | 38182830 | 38183430 | 0.078 | 0.028 | 0.03 | 0.75 | 0.79 | 0.859 |
| 5032 | chr7 | 1.54E+08 | 1.54E+08 | 0.114 | 0.029 | 0.027 | 0.75 | 0.83 | 0.858 |
| 5033 | chr7 | 1.54E+08 | 1.54E+08 | 0.114 | 0.029 | 0.027 | 0.75 | 0.83 | 0.858 |
| 5034 | chr19 | 56904601 | 56905201 | 0.105 | 0.038 | 0.041 | 0.75 | 0.74 | 0.858 |
| 5035 | chr19 | 56904665 | 56905265 | 0.105 | 0.038 | 0.041 | 0.75 | 0.74 | 0.858 |
| 5036 | chr19 | 38182953 | 38183553 | 0.075 | 0.025 | 0.026 | 0.75 | 0.8 | 0.858 |
| 5037 | chr19 | 38182955 | 38183555 | 0.075 | 0.025 | 0.026 | 0.75 | 0.8 | 0.858 |
| 5038 | chr19 | 38182962 | 38183562 | 0.075 | 0.025 | 0.026 | 0.75 | 0.8 | 0.858 |
| 5039 | chr19 | 56904645 | 56905245 | 0.105 | 0.038 | 0.041 | 0.75 | 0.74 | 0.858 |
| 5040 | chr19 | 56904677 | 56905277 | 0.105 | 0.038 | 0.041 | 0.75 | 0.74 | 0.858 |
| 5041 | chr19 | 38182959 | 38183559 | 0.075 | 0.025 | 0.026 | 0.75 | 0.8 | 0.858 |
| 5042 | chr19 | 56904713 | 56905313 | 0.105 | 0.038 | 0.041 | 0.75 | 0.74 | 0.858 |
| 5043 | chr19 | 56904697 | 56905297 | 0.105 | 0.038 | 0.041 | 0.75 | 0.74 | 0.858 |
| 5044 | chr19 | 56904732 | 56905332 | 0.105 | 0.038 | 0.041 | 0.75 | 0.74 | 0.858 |
| 5045 | chr19 | 56904455 | 56905055 | 0.109 | 0.039 | 0.041 | 0.75 | 0.76 | 0.858 |
| 5046 | chr12 | 1.29E+08 | 1.29E+08 | 0.09 | 0.034 | 0.038 | 0.75 | 0.74 | 0.858 |
| 5047 | chr19 | 38182910 | 38183510 | 0.075 | 0.027 | 0.029 | 0.75 | 0.77 | 0.856 |
| 5048 | chr7 | 1.54E+08 | 1.54E+08 | 0.111 | 0.029 | 0.028 | 0.75 | 0.83 | 0.855 |
| 5049 | chr7 | 1.54E+08 | 1.54E+08 | 0.111 | 0.029 | 0.028 | 0.75 | 0.83 | 0.855 |
| 5050 | chr7 | 1.54E+08 | 1.54E+08 | 0.111 | 0.029 | 0.028 | 0.75 | 0.83 | 0.855 |
| 5051 | chr12 | 1.03E+08 | 1.03E+08 | 0.076 | 0.032 | 0.034 | 0.75 | 0.76 | 0.852 |
| 5052 | chr12 | 1.03E+08 | 1.03E+08 | 0.076 | 0.032 | 0.034 | 0.75 | 0.76 | 0.852 |
| 5053 | chr11 | 1.32E+08 | 1.32E+08 | 0.133 | 0.035 | 0.036 | 0.75 | 0.82 | 0.851 |
| 5054 | chr11 | 1.32E+08 | 1.32E+08 | 0.133 | 0.035 | 0.036 | 0.75 | 0.82 | 0.851 |
| 5055 | chr1 | 2.14E+08 | 2.14E+08 | 0.104 | 0.045 | 0.05 | 0.75 | 0.76 | 0.851 |
| 5056 | chr18 | 5629716 | 5630316 | 0.078 | 0.016 | 0.016 | 0.75 | 0.77 | 0.851 |
| 5057 | chr11 | 1.32E+08 | 1.32E+08 | 0.133 | 0.035 | 0.036 | 0.75 | 0.82 | 0.851 |
| 5058 | chr19 | 57617836 | 57618436 | 0.184 | 0.073 | 0.085 | 0.75 | 0.77 | 0.848 |
| 5059 | chr18 | 5543248 | 5543848 | 0.102 | 0.028 | 0.028 | 0.75 | 0.8 | 0.847 |
| 5060 | chr10 | 1.11E+08 | 1.11E+08 | 0.116 | 0.041 | 0.042 | 0.75 | 0.8 | 0.847 |
| 5061 | chr12 | 1.03E+08 | 1.03E+08 | 0.088 | 0.034 | 0.037 | 0.75 | 0.77 | 0.846 |
| 5062 | chr2 | 5836757 | 5837357 | 0.117 | 0.053 | 0.057 | 0.75 | 0.77 | 0.846 |
| 5063 | chr18 | 5543645 | 5544245 | 0.077 | 0.031 | 0.031 | 0.75 | 0.77 | 0.845 |
| 5064 | chr18 | 5543731 | 5544331 | 0.077 | 0.031 | 0.031 | 0.75 | 0.77 | 0.845 |
| 5065 | chr18 | 5543799 | 5544399 | 0.077 | 0.031 | 0.031 | 0.75 | 0.77 | 0.845 |
| 5066 | chr18 | 5543789 | 5544389 | 0.077 | 0.031 | 0.031 | 0.75 | 0.77 | 0.845 |
| 5067 | chr12 | 1.03E+08 | 1.03E+08 | 0.089 | 0.038 | 0.041 | 0.75 | 0.77 | 0.843 |
| 5068 | chr14 | 48143659 | 48144259 | 0.097 | 0.035 | 0.037 | 0.75 | 0.79 | 0.843 |
| 5069 | chr18 | 70534234 | 70534834 | 0.126 | 0.047 | 0.05 | 0.75 | 0.82 | 0.84 |
| 5070 | chr2 | 5837048 | 5837648 | 0.101 | 0.045 | 0.049 | 0.75 | 0.77 | 0.84 |
| 5071 | chr6 | 1.23E+08 | 1.23E+08 | 0.101 | 0.038 | 0.041 | 0.75 | 0.7 | 0.84 |
| 5072 | chr18 | 70533998 | 70534598 | 0.129 | 0.053 | 0.06 | 0.75 | 0.77 | 0.839 |
| 5073 | chr14 | 38724375 | 38724975 | 0.124 | 0.038 | 0.042 | 0.75 | 0.79 | 0.839 |
| 5074 | chr14 | 38724348 | 38724948 | 0.124 | 0.038 | 0.042 | 0.75 | 0.79 | 0.839 |
| 5075 | chr14 | 38724346 | 38724946 | 0.124 | 0.038 | 0.042 | 0.75 | 0.79 | 0.839 |
| 5076 | chr14 | 38724645 | 38725245 | 0.124 | 0.038 | 0.042 | 0.75 | 0.79 | 0.839 |
| 5077 | chr4 | 21950007 | 21950607 | 0.093 | 0.037 | 0.038 | 0.75 | 0.77 | 0.839 |
| 5078 | chr2 | 5836413 | 5837013 | 0.114 | 0.022 | 0.016 | 0.75 | 0.77 | 0.838 |
| 5079 | chr18 | 5543578 | 5544178 | 0.084 | 0.032 | 0.033 | 0.75 | 0.77 | 0.838 |
| 5080 | chr18 | 5543869 | 5544469 | 0.074 | 0.031 | 0.032 | 0.75 | 0.77 | 0.838 |
| 5081 | chr10 | 1.11E+08 | 1.11E+08 | 0.081 | 0.035 | 0.037 | 0.75 | 0.75 | 0.838 |
| 5082 | chr11 | 20690640 | 20691240 | 0.104 | 0.032 | 0.033 | 0.75 | 0.75 | 0.837 |
| 5083 | chr6 | 1.23E+08 | 1.23E+08 | 0.093 | 0.038 | 0.042 | 0.75 | 0.71 | 0.837 |
| 5084 | chr11 | 20690630 | 20691230 | 0.104 | 0.032 | 0.033 | 0.75 | 0.75 | 0.837 |
| 5085 | chr11 | 20690657 | 20691257 | 0.104 | 0.032 | 0.033 | 0.75 | 0.75 | 0.837 |
| 5086 | chr11 | 20690634 | 20691234 | 0.104 | 0.032 | 0.033 | 0.75 | 0.75 | 0.837 |
| 5087 | chr5 | 1.14E+08 | 1.14E+08 | 0.112 | 0.052 | 0.058 | 0.75 | 0.78 | 0.837 |
| 5088 | chr20 | 21686248 | 21686848 | 0.082 | 0.038 | 0.042 | 0.75 | 0.74 | 0.837 |
| 5089 | chr10 | 1.11E+08 | 1.11E+08 | 0.075 | 0.033 | 0.036 | 0.75 | 0.77 | 0.836 |
| 5090 | chr6 | 1.23E+08 | 1.23E+08 | 0.092 | 0.037 | 0.04 | 0.75 | 0.7 | 0.836 |
| 5091 | chr6 | 1.23E+08 | 1.23E+08 | 0.092 | 0.037 | 0.04 | 0.75 | 0.7 | 0.836 |
| 5092 | chr6 | 1.23E+08 | 1.23E+08 | 0.098 | 0.039 | 0.042 | 0.75 | 0.7 | 0.836 |
| 5093 | chr4 | 21950267 | 21950867 | 0.111 | 0.046 | 0.05 | 0.75 | 0.79 | 0.835 |
| 5094 | chr4 | 21950284 | 21950884 | 0.111 | 0.046 | 0.05 | 0.75 | 0.79 | 0.835 |
| 5095 | chr19 | 30865902 | 30866502 | 0.116 | 0.032 | 0.032 | 0.75 | 0.77 | 0.834 |
| 5096 | chr19 | 30865894 | 30866494 | 0.116 | 0.032 | 0.032 | 0.75 | 0.77 | 0.834 |
| 5097 | chr7 | 70597413 | 70598013 | 0.111 | 0.051 | 0.053 | 0.75 | 0.77 | 0.834 |
| 5098 | chr11 | 1.33E+08 | 1.33E+08 | 0.067 | 0.023 | 0.024 | 0.75 | 0.75 | 0.834 |
| 5099 | chr19 | 30866065 | 30866665 | 0.116 | 0.032 | 0.032 | 0.75 | 0.77 | 0.834 |
| 5100 | chr11 | 20690382 | 20690982 | 0.102 | 0.033 | 0.036 | 0.75 | 0.72 | 0.834 |
| 5101 | chr11 | 20690880 | 20691480 | 0.104 | 0.034 | 0.034 | 0.75 | 0.76 | 0.834 |
| 5102 | chr19 | 30865892 | 30866492 | 0.116 | 0.032 | 0.032 | 0.75 | 0.77 | 0.834 |
| 5103 | chr11 | 20690861 | 20691461 | 0.104 | 0.034 | 0.034 | 0.75 | 0.76 | 0.834 |
| 5104 | chr11 | 1.33E+08 | 1.33E+08 | 0.067 | 0.023 | 0.024 | 0.75 | 0.75 | 0.834 |
| 5105 | chr11 | 1.33E+08 | 1.33E+08 | 0.067 | 0.023 | 0.024 | 0.75 | 0.75 | 0.834 |
| 5106 | chr11 | 20690507 | 20691107 | 0.102 | 0.033 | 0.036 | 0.75 | 0.72 | 0.834 |
| 5107 | chr11 | 20690420 | 20691020 | 0.102 | 0.033 | 0.036 | 0.75 | 0.72 | 0.834 |
| 5108 | chr10 | 1.11E+08 | 1.11E+08 | 0.111 | 0.048 | 0.051 | 0.75 | 0.77 | 0.833 |
| 5109 | chr19 | 58010840 | 58011440 | 0.107 | 0.037 | 0.04 | 0.75 | 0.76 | 0.833 |
| 5110 | chr19 | 58010967 | 58011567 | 0.107 | 0.037 | 0.04 | 0.75 | 0.76 | 0.833 |
| 5111 | chr7 | 70597478 | 70598078 | 0.117 | 0.054 | 0.057 | 0.75 | 0.77 | 0.833 |
| 5112 | chr19 | 58011009 | 58011609 | 0.107 | 0.037 | 0.04 | 0.75 | 0.76 | 0.833 |
| 5113 | chr7 | 70597474 | 70598074 | 0.117 | 0.054 | 0.057 | 0.75 | 0.77 | 0.833 |
| 5114 | chr19 | 58011045 | 58011645 | 0.107 | 0.037 | 0.04 | 0.75 | 0.76 | 0.833 |
| 5115 | chr11 | 20690826 | 20691426 | 0.112 | 0.036 | 0.037 | 0.75 | 0.76 | 0.833 |
| 5116 | chr12 | 1.29E+08 | 1.29E+08 | 0.136 | 0.043 | 0.047 | 0.75 | 0.76 | 0.831 |
| 5117 | chr16 | 62069705 | 62070305 | 0.078 | 0.033 | 0.035 | 0.75 | 0.78 | 0.831 |
| 5118 | chr20 | 21685973 | 21686573 | 0.083 | 0.038 | 0.043 | 0.75 | 0.74 | 0.83 |
| 5119 | chr20 | 21685993 | 21686593 | 0.083 | 0.038 | 0.043 | 0.75 | 0.74 | 0.83 |
| 5120 | chr20 | 21685982 | 21686582 | 0.083 | 0.038 | 0.043 | 0.75 | 0.74 | 0.83 |
| 5121 | chr20 | 21685964 | 21686564 | 0.083 | 0.038 | 0.043 | 0.75 | 0.74 | 0.83 |
| 5122 | chr5 | 11384765 | 11385365 | 0.06 | 0.021 | 0.022 | 0.75 | 0.75 | 0.83 |
| 5123 | chr20 | 21685951 | 21686551 | 0.083 | 0.038 | 0.043 | 0.75 | 0.74 | 0.83 |
| 5124 | chr20 | 21686076 | 21686676 | 0.083 | 0.038 | 0.043 | 0.75 | 0.74 | 0.83 |
| 5125 | chr20 | 21686008 | 21686608 | 0.083 | 0.038 | 0.043 | 0.75 | 0.74 | 0.83 |
| 5126 | chr5 | 11384767 | 11385367 | 0.06 | 0.021 | 0.022 | 0.75 | 0.75 | 0.83 |
| 5127 | chr20 | 21685953 | 21686553 | 0.083 | 0.038 | 0.043 | 0.75 | 0.74 | 0.83 |
| 5128 | chr7 | 35293569 | 35294169 | 0.097 | 0.037 | 0.038 | 0.75 | 0.7 | 0.829 |
| 5129 | chr7 | 35293592 | 35294192 | 0.097 | 0.037 | 0.038 | 0.75 | 0.7 | 0.829 |
| 5130 | chr16 | 62069506 | 62070106 | 0.076 | 0.027 | 0.029 | 0.75 | 0.72 | 0.829 |
| 5131 | chr7 | 35293584 | 35294184 | 0.097 | 0.037 | 0.038 | 0.75 | 0.7 | 0.829 |
| 5132 | chr12 | 1.15E+08 | 1.15E+08 | 0.068 | 0.028 | 0.03 | 0.75 | 0.77 | 0.828 |
| 5133 | chr1 | 2.37E+08 | 2.37E+08 | 0.085 | 0.033 | 0.036 | 0.75 | 0.72 | 0.828 |
| 5134 | chr5 | 1.14E+08 | 1.14E+08 | 0.096 | 0.047 | 0.051 | 0.75 | 0.78 | 0.828 |
| 5135 | chr2 | 47797663 | 47798263 | 0.082 | 0.024 | 0.025 | 0.75 | 0.76 | 0.828 |
| 5136 | chr1 | 2.37E+08 | 2.37E+08 | 0.085 | 0.033 | 0.036 | 0.75 | 0.72 | 0.828 |
| 5137 | chr13 | 1.13E+08 | 1.13E+08 | 0.169 | 0.082 | 0.088 | 0.75 | 0.77 | 0.827 |
| 5138 | chr13 | 1.13E+08 | 1.13E+08 | 0.169 | 0.082 | 0.088 | 0.75 | 0.77 | 0.827 |
| 5139 | chr13 | 1.09E+08 | 1.09E+08 | 0.082 | 0.037 | 0.04 | 0.75 | 0.76 | 0.827 |
| 5140 | chr1 | 50513466 | 50514066 | 0.161 | 0.091 | 0.099 | 0.75 | 0.74 | 0.826 |
| 5141 | chr13 | 1.09E+08 | 1.09E+08 | 0.088 | 0.04 | 0.044 | 0.75 | 0.74 | 0.826 |
| 5142 | chr13 | 1.13E+08 | 1.13E+08 | 0.067 | 0.026 | 0.029 | 0.75 | 0.7 | 0.826 |
| 5143 | chr4 | 21950422 | 21951022 | 0.128 | 0.062 | 0.07 | 0.75 | 0.74 | 0.826 |
| 5144 | chr18 | 5629772 | 5630372 | 0.075 | 0.019 | 0.02 | 0.75 | 0.71 | 0.826 |
| 5145 | chr13 | 1.13E+08 | 1.13E+08 | 0.067 | 0.026 | 0.029 | 0.75 | 0.7 | 0.826 |
| 5146 | chr18 | 70533810 | 70534410 | 0.138 | 0.058 | 0.065 | 0.75 | 0.77 | 0.826 |
| 5147 | chr16 | 6069101 | 6069701 | 0.154 | 0.077 | 0.084 | 0.75 | 0.76 | 0.825 |
| 5148 | chr16 | 6069111 | 6069711 | 0.154 | 0.077 | 0.084 | 0.75 | 0.76 | 0.825 |
| 5149 | chr16 | 6069099 | 6069699 | 0.154 | 0.077 | 0.084 | 0.75 | 0.76 | 0.825 |
| 5150 | chr14 | 48143897 | 48144497 | 0.108 | 0.053 | 0.058 | 0.75 | 0.72 | 0.825 |
| 5151 | chr14 | 48143886 | 48144486 | 0.108 | 0.053 | 0.058 | 0.75 | 0.72 | 0.825 |
| 5152 | chr10 | 44880408 | 44881008 | 0.082 | 0.025 | 0.027 | 0.75 | 0.74 | 0.825 |
| 5153 | chr10 | 44880430 | 44881030 | 0.082 | 0.025 | 0.027 | 0.75 | 0.74 | 0.825 |
| 5154 | chr16 | 62069919 | 62070519 | 0.081 | 0.039 | 0.042 | 0.75 | 0.71 | 0.825 |
| 5155 | chr16 | 62069946 | 62070546 | 0.081 | 0.039 | 0.042 | 0.75 | 0.71 | 0.825 |
| 5156 | chr12 | 1.15E+08 | 1.15E+08 | 0.067 | 0.032 | 0.034 | 0.75 | 0.74 | 0.825 |
| 5157 | chr16 | 62069884 | 62070484 | 0.081 | 0.039 | 0.042 | 0.75 | 0.71 | 0.825 |
| 5158 | chr16 | 62069864 | 62070464 | 0.081 | 0.039 | 0.042 | 0.75 | 0.71 | 0.825 |
| 5159 | chr10 | 44880245 | 44880845 | 0.082 | 0.025 | 0.027 | 0.75 | 0.74 | 0.825 |
| 5160 | chr12 | 1.15E+08 | 1.15E+08 | 0.067 | 0.032 | 0.034 | 0.75 | 0.74 | 0.825 |
| 5161 | chr10 | 44880242 | 44880842 | 0.082 | 0.025 | 0.027 | 0.75 | 0.74 | 0.825 |
| 5162 | chr10 | 44880262 | 44880862 | 0.082 | 0.025 | 0.027 | 0.75 | 0.74 | 0.825 |
| 5163 | chr10 | 44880259 | 44880859 | 0.082 | 0.025 | 0.027 | 0.75 | 0.74 | 0.825 |
| 5164 | chr16 | 6069366 | 6069966 | 0.164 | 0.087 | 0.095 | 0.75 | 0.76 | 0.824 |
| 5165 | chr1 | 50513627 | 50514227 | 0.124 | 0.05 | 0.054 | 0.75 | 0.76 | 0.823 |
| 5166 | chr16 | 62069786 | 62070386 | 0.081 | 0.039 | 0.042 | 0.75 | 0.71 | 0.823 |
| 5167 | chr1 | 50513570 | 50514170 | 0.124 | 0.05 | 0.054 | 0.75 | 0.76 | 0.823 |
| 5168 | chr14 | 38678237 | 38678837 | 0.09 | 0.047 | 0.051 | 0.75 | 0.75 | 0.823 |
| 5169 | chr14 | 38678120 | 38678720 | 0.09 | 0.047 | 0.051 | 0.75 | 0.75 | 0.823 |
| 5170 | chr8 | 73163719 | 73164319 | 0.077 | 0.031 | 0.035 | 0.75 | 0.75 | 0.823 |
| 5171 | chr8 | 73163724 | 73164324 | 0.077 | 0.031 | 0.035 | 0.75 | 0.75 | 0.823 |
| 5172 | chr14 | 38678245 | 38678845 | 0.09 | 0.047 | 0.051 | 0.75 | 0.75 | 0.823 |
| 5173 | chr16 | 62069772 | 62070372 | 0.081 | 0.039 | 0.042 | 0.75 | 0.71 | 0.823 |
| 5174 | chr7 | 70597621 | 70598221 | 0.115 | 0.054 | 0.056 | 0.75 | 0.77 | 0.822 |
| 5175 | chr14 | 48144069 | 48144669 | 0.116 | 0.054 | 0.059 | 0.75 | 0.72 | 0.822 |
| 5176 | chr14 | 48144014 | 48144614 | 0.116 | 0.054 | 0.059 | 0.75 | 0.72 | 0.822 |
| 5177 | chr14 | 48144039 | 48144639 | 0.116 | 0.054 | 0.059 | 0.75 | 0.72 | 0.822 |
| 5178 | chr1 | 77333449 | 77334049 | 0.066 | 0.018 | 0.021 | 0.75 | 0.68 | 0.822 |
| 5179 | chr14 | 48143980 | 48144580 | 0.116 | 0.054 | 0.059 | 0.75 | 0.72 | 0.822 |
| 5180 | chr4 | 21950466 | 21951066 | 0.122 | 0.062 | 0.07 | 0.75 | 0.75 | 0.821 |
| 5181 | chr19 | 21769015 | 21769615 | 0.13 | 0.037 | 0.04 | 0.75 | 0.76 | 0.821 |
| 5182 | chr5 | 1.29E+08 | 1.29E+08 | 0.077 | 0.038 | 0.042 | 0.75 | 0.7 | 0.821 |
| 5183 | chr9 | 1.05E+08 | 1.05E+08 | 0.08 | 0.035 | 0.038 | 0.75 | 0.76 | 0.821 |
| 5184 | chr5 | 1.29E+08 | 1.29E+08 | 0.077 | 0.038 | 0.042 | 0.75 | 0.7 | 0.821 |
| 5185 | chr4 | 21950564 | 21951164 | 0.122 | 0.062 | 0.07 | 0.75 | 0.75 | 0.821 |
| 5186 | chr19 | 21769130 | 21769730 | 0.13 | 0.037 | 0.04 | 0.75 | 0.76 | 0.821 |
| 5187 | chr19 | 21768890 | 21769490 | 0.13 | 0.037 | 0.04 | 0.75 | 0.76 | 0.821 |
| 5188 | chr14 | 29243104 | 29243704 | 0.112 | 0.072 | 0.078 | 0.75 | 0.75 | 0.821 |
| 5189 | chr9 | 1.05E+08 | 1.05 E+08 | 0.08 | 0.035 | 0.038 | 0.75 | 0.76 | 0.821 |
| 5190 | chr13 | 1.13E+08 | 1.13E+08 | 0.122 | 0.021 | 0.019 | 0.75 | 0.75 | 0.82 |
| 5191 | chr7 | 70597054 | 70597654 | 0.087 | 0.035 | 0.037 | 0.75 | 0.75 | 0.82 |
| 5192 | chr7 | 70597051 | 70597651 | 0.087 | 0.035 | 0.037 | 0.75 | 0.75 | 0.82 |
| 5193 | chr10 | 1.09E+08 | 1.09E+08 | 0.09 | 0.027 | 0.029 | 0.75 | 0.72 | 0.82 |
| 5194 | chr18 | 70209132 | 70209732 | 0.109 | 0.049 | 0.054 | 0.75 | 0.77 | 0.819 |
| 5195 | chr10 | 1.06E+08 | 1.06E+08 | 0.087 | 0.03 | 0.031 | 0.75 | 0.74 | 0.819 |
| 5196 | chr19 | 58609318 | 58609918 | 0.088 | 0.023 | 0.024 | 0.75 | 0.71 | 0.819 |
| 5197 | chr10 | 1.09E+08 | 1.09E+08 | 0.088 | 0.028 | 0.03 | 0.75 | 0.71 | 0.819 |
| 5198 | chr19 | 58609302 | 58609902 | 0.088 | 0.023 | 0.024 | 0.75 | 0.71 | 0.819 |
| 5199 | chr10 | 1.09E+08 | 1.09E+08 | 0.088 | 0.028 | 0.03 | 0.75 | 0.71 | 0.819 |
| 5200 | chr10 | 1.06E+08 | 1.06E+08 | 0.087 | 0.03 | 0.031 | 0.75 | 0.74 | 0.819 |
| 5201 | chr10 | 1.06E+08 | 1.06E+08 | 0.087 | 0.03 | 0.031 | 0.75 | 0.74 | 0.819 |
| 5202 | chr18 | 70209251 | 70209851 | 0.109 | 0.049 | 0.054 | 0.75 | 0.77 | 0.819 |
| 5203 | chr10 | 1.09E+08 | 1.09E+08 | 0.088 | 0.028 | 0.03 | 0.75 | 0.71 | 0.819 |
| 5204 | chr13 | 28366514 | 28367114 | 0.077 | 0.025 | 0.026 | 0.75 | 0.7 | 0.818 |
| 5205 | chr19 | 30015847 | 30016447 | 0.093 | 0.021 | 0.022 | 0.75 | 0.75 | 0.818 |
| 5206 | chr18 | 5543908 | 5544508 | 0.072 | 0.031 | 0.032 | 0.75 | 0.72 | 0.818 |
| 5207 | chr19 | 30015836 | 30016436 | 0.093 | 0.021 | 0.022 | 0.75 | 0.75 | 0.818 |
| 5208 | chr18 | 5543931 | 5544531 | 0.072 | 0.031 | 0.032 | 0.75 | 0.72 | 0.818 |
| 5209 | chr3 | 1.8E+08 | 1.8E+08 | 0.086 | 0.037 | 0.039 | 0.75 | 0.75 | 0.818 |
| 5210 | chr10 | 1.11E+08 | 1.11E+08 | 0.073 | 0.033 | 0.036 | 0.75 | 0.74 | 0.818 |
| 5211 | chr5 | 1.48E+08 | 1.48E+08 | 0.133 | 0.06 | 0.066 | 0.75 | 0.69 | 0.817 |
| 5212 | chr7 | 1.54E+08 | 1.54E+08 | 0.117 | 0.042 | 0.044 | 0.75 | 0.72 | 0.817 |
| 5213 | chr7 | 1.54E+08 | 1.54E+08 | 0.117 | 0.042 | 0.044 | 0.75 | 0.72 | 0.817 |
| 5214 | chr7 | 1.54E+08 | 1.54E+08 | 0.117 | 0.042 | 0.044 | 0.75 | 0.72 | 0.817 |
| 5215 | chr8 | 35093601 | 35094201 | 0.083 | 0.039 | 0.042 | 0.75 | 0.75 | 0.817 |
| 5216 | chr5 | 1.48E+08 | 1.48E+08 | 0.133 | 0.06 | 0.066 | 0.75 | 0.69 | 0.817 |
| 5217 | chr5 | 1.29E+08 | 1.29E+08 | 0.059 | 0.025 | 0.027 | 0.75 | 0.74 | 0.817 |
| 5218 | chr12 | 1.15E+08 | 1.15E+08 | 0.054 | 0.021 | 0.022 | 0.75 | 0.72 | 0.817 |
| 5219 | chr5 | 1.29E+08 | 1.29E+08 | 0.059 | 0.025 | 0.027 | 0.75 | 0.74 | 0.817 |
| 5220 | chr5 | 1.29E+08 | 1.29E+08 | 0.059 | 0.025 | 0.027 | 0.75 | 0.74 | 0.817 |
| 5221 | chr7 | 70597299 | 70597899 | 0.101 | 0.048 | 0.051 | 0.75 | 0.71 | 0.816 |
| 5222 | chr16 | 62070006 | 62070606 | 0.084 | 0.041 | 0.044 | 0.75 | 0.71 | 0.816 |
| 5223 | chr16 | 62070065 | 62070665 | 0.084 | 0.041 | 0.044 | 0.75 | 0.71 | 0.816 |
| 5224 | chr6 | 1.23E+08 | 1.23E+08 | 0.095 | 0.045 | 0.049 | 0.75 | 0.72 | 0.815 |
| 5225 | chr3 | 1.47E+08 | 1.47E+08 | 0.092 | 0.041 | 0.044 | 0.75 | 0.74 | 0.815 |
| 5226 | chr6 | 1.23E+08 | 1.23E+08 | 0.095 | 0.045 | 0.049 | 0.75 | 0.72 | 0.815 |
| 5227 | chr10 | 1.09E+08 | 1.09E+08 | 0.094 | 0.03 | 0.033 | 0.75 | 0.69 | 0.814 |
| 5228 | chr13 | 37247944 | 37248544 | 0.09 | 0.024 | 0.023 | 0.75 | 0.74 | 0.814 |
| 5229 | chr10 | 1.09E+08 | 1.09E+08 | 0.094 | 0.03 | 0.033 | 0.75 | 0.69 | 0.814 |
| 5230 | chr10 | 1.09E+08 | 1.09E+08 | 0.094 | 0.03 | 0.033 | 0.75 | 0.69 | 0.814 |
| 5231 | chr7 | 32110373 | 32110973 | 0.056 | 0.021 | 0.023 | 0.75 | 0.71 | 0.814 |
| 5232 | chr16 | 6068898 | 6069498 | 0.129 | 0.073 | 0.079 | 0.75 | 0.7 | 0.814 |
| 5233 | chr7 | 32110350 | 32110950 | 0.056 | 0.021 | 0.023 | 0.75 | 0.71 | 0.814 |
| 5234 | chr12 | 43945380 | 43945980 | 0.099 | 0.035 | 0.04 | 0.75 | 0.71 | 0.814 |
| 5235 | chr12 | 1.29E+08 | 1.29E+08 | 0.091 | 0.044 | 0.051 | 0.75 | 0.72 | 0.814 |
| 5236 | chr7 | 32110336 | 32110936 | 0.056 | 0.021 | 0.023 | 0.75 | 0.71 | 0.814 |
| 5237 | chr10 | 1.09E+08 | 1.09E+08 | 0.094 | 0.03 | 0.033 | 0.75 | 0.69 | 0.814 |
| 5238 | chr2 | 47797956 | 47798556 | 0.103 | 0.044 | 0.048 | 0.75 | 0.67 | 0.813 |
| 5239 | chr19 | 23257796 | 23258396 | 0.113 | 0.058 | 0.063 | 0.75 | 0.75 | 0.813 |
| 5240 | chr19 | 58446136 | 58446736 | 0.087 | 0.045 | 0.05 | 0.75 | 0.74 | 0.813 |
| 5241 | chr19 | 58570490 | 58571090 | 0.075 | 0.031 | 0.035 | 0.75 | 0.76 | 0.813 |
| 5242 | chr10 | 1.11E+08 | 1.11E+08 | 0.106 | 0.053 | 0.058 | 0.75 | 0.69 | 0.812 |
| 5243 | chr14 | 52734229 | 52734829 | 0.091 | 0.037 | 0.039 | 0.75 | 0.71 | 0.812 |
| 5244 | chr19 | 56915103 | 56915703 | 0.072 | 0.026 | 0.028 | 0.75 | 0.67 | 0.812 |
| 5245 | chr9 | 1.05E+08 | 1.05E+08 | 0.071 | 0.03 | 0.034 | 0.75 | 0.69 | 0.812 |
| 5246 | chr13 | 37247778 | 37248378 | 0.094 | 0.024 | 0.022 | 0.75 | 0.76 | 0.812 |
| 5247 | chr14 | 52734225 | 52734825 | 0.091 | 0.037 | 0.039 | 0.75 | 0.71 | 0.812 |
| 5248 | chr1 | 50513449 | 50514049 | 0.24 | 0.176 | 0.187 | 0.75 | 0.67 | 0.811 |
| 5249 | chr9 | 1.05E+08 | 1.05E+08 | 0.084 | 0.038 | 0.042 | 0.75 | 0.71 | 0.811 |
| 5250 | chr9 | 1.05E+08 | 1.05E+08 | 0.084 | 0.038 | 0.042 | 0.75 | 0.71 | 0.811 |
| 5251 | chr18 | 904551 | 905151 | 0.098 | 0.052 | 0.06 | 0.75 | 0.67 | 0.811 |
| 5252 | chr19 | 58545033 | 58545633 | 0.092 | 0.037 | 0.04 | 0.75 | 0.68 | 0.81 |
| 5253 | chr8 | 69242985 | 69243585 | 0.061 | 0.017 | 0.018 | 0.75 | 0.66 | 0.81 |
| 5254 | chr7 | 32110223 | 32110823 | 0.055 | 0.024 | 0.026 | 0.75 | 0.72 | 0.81 |
| 5255 | chr7 | 32110225 | 32110825 | 0.055 | 0.024 | 0.026 | 0.75 | 0.72 | 0.81 |
| 5256 | chr13 | 1.09E+08 | 1.09E+08 | 0.089 | 0.046 | 0.051 | 0.75 | 0.69 | 0.81 |
| 5257 | chr13 | 1.09E+08 | 1.09E+08 | 0.089 | 0.046 | 0.051 | 0.75 | 0.69 | 0.81 |
| 5258 | chr13 | 1.13E+08 | 1.13E+08 | 0.113 | 0.046 | 0.051 | 0.75 | 0.7 | 0.809 |
| 5259 | chr10 | 1.1E+08 | 1.1E+08 | 0.154 | 0.071 | 0.091 | 0.75 | 0.72 | 0.809 |
| 5260 | chr5 | 1.14E+08 | 1.14E+08 | 0.083 | 0.04 | 0.044 | 0.75 | 0.7 | 0.809 |
| 5261 | chr13 | 1.13E+08 | 1.13E+08 | 0.113 | 0.046 | 0.051 | 0.75 | 0.7 | 0.809 |
| 5262 | chr1 | 1.71E+08 | 1.71E+08 | 0.063 | 0.026 | 0.029 | 0.75 | 0.74 | 0.809 |
| 5263 | chr16 | 20084899 | 20085499 | 0.085 | 0.036 | 0.039 | 0.75 | 0.72 | 0.809 |
| 5264 | chr7 | 70597140 | 70597740 | 0.101 | 0.045 | 0.047 | 0.75 | 0.71 | 0.808 |
| 5265 | chr12 | 43945574 | 43946174 | 0.102 | 0.042 | 0.046 | 0.75 | 0.71 | 0.808 |
| 5266 | chr18 | 67067593 | 67068193 | 0.067 | 0.022 | 0.023 | 0.75 | 0.72 | 0.808 |
| 5267 | chr7 | 70597158 | 70597758 | 0.101 | 0.045 | 0.047 | 0.75 | 0.71 | 0.808 |
| 5268 | chr8 | 22089144 | 22089744 | 0.127 | 0.066 | 0.078 | 0.75 | 0.7 | 0.808 |
| 5269 | chr3 | 1.47E+08 | 1.47E+08 | 0.14 | 0.06 | 0.065 | 0.75 | 0.75 | 0.808 |
| 5270 | chr18 | 67067421 | 67068021 | 0.067 | 0.022 | 0.023 | 0.75 | 0.72 | 0.808 |
| 5271 | chr18 | 67067567 | 67068167 | 0.067 | 0.022 | 0.023 | 0.75 | 0.72 | 0.808 |
| 5272 | chr5 | 63801980 | 63802580 | 0.087 | 0.046 | 0.051 | 0.75 | 0.69 | 0.808 |
| 5273 | chr5 | 63801972 | 63802572 | 0.087 | 0.046 | 0.051 | 0.75 | 0.69 | 0.808 |
| 5274 | chr7 | 8473690 | 8474290 | 0.112 | 0.06 | 0.068 | 0.75 | 0.68 | 0.807 |
| 5275 | chr19 | 30017211 | 30017811 | 0.125 | 0.051 | 0.057 | 0.75 | 0.72 | 0.807 |
| 5276 | chr7 | 8473878 | 8474478 | 0.112 | 0.06 | 0.068 | 0.75 | 0.68 | 0.807 |
| 5277 | chr7 | 8473876 | 8474476 | 0.112 | 0.06 | 0.068 | 0.75 | 0.68 | 0.807 |
| 5278 | chr13 | 88324132 | 88324732 | 0.082 | 0.032 | 0.035 | 0.75 | 0.71 | 0.807 |
| 5279 | chr3 | 1.8E+08 | 1.8E+08 | 0.081 | 0.036 | 0.039 | 0.75 | 0.74 | 0.807 |
| 5280 | chr7 | 8473845 | 8474445 | 0.112 | 0.06 | 0.068 | 0.75 | 0.68 | 0.807 |
| 5281 | chr3 | 1.8E+08 | 1.8E+08 | 0.081 | 0.036 | 0.039 | 0.75 | 0.74 | 0.807 |
| 5282 | chr19 | 56915222 | 56915822 | 0.077 | 0.025 | 0.028 | 0.75 | 0.68 | 0.807 |
| 5283 | chr10 | 1.11E+08 | 1.11E+08 | 0.078 | 0.038 | 0.042 | 0.75 | 0.74 | 0.807 |
| 5284 | chr12 | 1.15E+08 | 1.15E+08 | 0.058 | 0.029 | 0.032 | 0.75 | 0.68 | 0.807 |
| 5285 | chr3 | 1.47E+08 | 1.47E+08 | 0.164 | 0.075 | 0.091 | 0.75 | 0.69 | 0.806 |
| 5286 | chr12 | 1.29E+08 | 1.29E+08 | 0.117 | 0.059 | 0.069 | 0.75 | 0.69 | 0.806 |
| 5287 | chr3 | 1.47E+08 | 1.47E+08 | 0.164 | 0.075 | 0.091 | 0.75 | 0.69 | 0.806 |
| 5288 | chr6 | 62995663 | 62996263 | 0.118 | 0.055 | 0.061 | 0.75 | 0.64 | 0.806 |
| 5289 | chr12 | 1.04E+08 | 1.04E+08 | 0.08 | 0.031 | 0.033 | 0.75 | 0.68 | 0.806 |
| 5290 | chr12 | 1.03E+08 | 1.03E+08 | 0.094 | 0.041 | 0.045 | 0.75 | 0.67 | 0.806 |
| 5291 | chr18 | 904578 | 905178 | 0.091 | 0.049 | 0.056 | 0.75 | 0.64 | 0.806 |
| 5292 | chr3 | 1.8E+08 | 1.8E+08 | 0.085 | 0.037 | 0.041 | 0.75 | 0.72 | 0.805 |
| 5293 | chr6 | 29521188 | 29521788 | 0.106 | 0.031 | 0.033 | 0.75 | 0.71 | 0.805 |
| 5294 | chr18 | 11148614 | 11149214 | 0.104 | 0.053 | 0.056 | 0.75 | 0.74 | 0.805 |
| 5295 | chr4 | 9782946 | 9783546 | 0.15 | 0.068 | 0.072 | 0.75 | 0.71 | 0.805 |
| 5296 | chr1 | 77333745 | 77334345 | 0.075 | 0.025 | 0.028 | 0.75 | 0.64 | 0.805 |
| 5297 | chr19 | 58609444 | 58610044 | 0.105 | 0.039 | 0.041 | 0.75 | 0.71 | 0.805 |
| 5298 | chr3 | 1.8E+08 | 1.8E+08 | 0.085 | 0.037 | 0.041 | 0.75 | 0.72 | 0.805 |
| 5299 | chr18 | 70534467 | 70535067 | 0.138 | 0.076 | 0.087 | 0.75 | 0.68 | 0.805 |
| 5300 | chr3 | 1.8E+08 | 1.8E+08 | 0.085 | 0.037 | 0.041 | 0.75 | 0.72 | 0.805 |
| 5301 | chr19 | 58609470 | 58610070 | 0.105 | 0.039 | 0.041 | 0.75 | 0.71 | 0.805 |
| 5302 | chr1 | 91190591 | 91191191 | 0.122 | 0.048 | 0.052 | 0.75 | 0.7 | 0.805 |
| 5303 | chr19 | 58609464 | 58610064 | 0.105 | 0.039 | 0.041 | 0.75 | 0.71 | 0.805 |
| 5304 | chr18 | 11148532 | 11149132 | 0.104 | 0.053 | 0.056 | 0.75 | 0.74 | 0.805 |
| 5305 | chr19 | 22805884 | 22806484 | 0.213 | 0.095 | 0.113 | 0.75 | 0.69 | 0.804 |
| 5306 | chr5 | 1.14E+08 | 1.14E+08 | 0.097 | 0.056 | 0.061 | 0.75 | 0.74 | 0.804 |
| 5307 | chr6 | 29521206 | 29521806 | 0.115 | 0.04 | 0.045 | 0.75 | 0.66 | 0.804 |
| 5308 | chr19 | 58570695 | 58571295 | 0.083 | 0.033 | 0.037 | 0.75 | 0.74 | 0.804 |
| 5309 | chr13 | 28366459 | 28367059 | 0.077 | 0.03 | 0.032 | 0.75 | 0.64 | 0.804 |
| 5310 | chr19 | 58570752 | 58571352 | 0.083 | 0.033 | 0.037 | 0.75 | 0.74 | 0.804 |
| 5311 | chr19 | 58609493 | 58610093 | 0.109 | 0.04 | 0.043 | 0.75 | 0.71 | 0.804 |
| 5312 | chr19 | 58609506 | 58610106 | 0.109 | 0.04 | 0.043 | 0.75 | 0.71 | 0.804 |
| 5313 | chr2 | 1.16E+08 | 1.16E+08 | 0.099 | 0.035 | 0.037 | 0.75 | 0.69 | 0.803 |
| 5314 | chr19 | 58545242 | 58545842 | 0.108 | 0.039 | 0.041 | 0.75 | 0.74 | 0.803 |
| 5315 | chr6 | 29521201 | 29521801 | 0.108 | 0.033 | 0.035 | 0.75 | 0.69 | 0.803 |
| 5316 | chr2 | 1.16E+08 | 1.16E+08 | 0.099 | 0.035 | 0.037 | 0.75 | 0.69 | 0.803 |
| 5317 | chr6 | 29521199 | 29521799 | 0.108 | 0.033 | 0.035 | 0.75 | 0.69 | 0.803 |
| 5318 | chr19 | 30015691 | 30016291 | 0.08 | 0.026 | 0.027 | 0.75 | 0.71 | 0.803 |
| 5319 | chr11 | 1.32E+08 | 1.32E+08 | 0.064 | 0.027 | 0.029 | 0.75 | 0.69 | 0.803 |
| 5320 | chr11 | 1.32E+08 | 1.32E+08 | 0.064 | 0.027 | 0.029 | 0.75 | 0.69 | 0.803 |
| 5321 | chr2 | 47798096 | 47798696 | 0.098 | 0.05 | 0.055 | 0.75 | 0.63 | 0.803 |
| 5322 | chr2 | 47798177 | 47798777 | 0.098 | 0.05 | 0.055 | 0.75 | 0.63 | 0.803 |
| 5323 | chr11 | 1.32E+08 | 1.32E+08 | 0.064 | 0.027 | 0.029 | 0.75 | 0.69 | 0.803 |
| 5324 | chr13 | 37248005 | 37248605 | 0.094 | 0.023 | 0.022 | 0.75 | 0.69 | 0.803 |
| 5325 | chr1 | 75602546 | 75603146 | 0.171 | 0.111 | 0.122 | 0.75 | 0.7 | 0.802 |
| 5326 | chr2 | 1.16E+08 | 1.16E+08 | 0.127 | 0.063 | 0.071 | 0.75 | 0.7 | 0.802 |
| 5327 | chr2 | 1.16E+08 | 1.16E+08 | 0.127 | 0.063 | 0.071 | 0.75 | 0.7 | 0.802 |
| 5328 | chr6 | 29521056 | 29521656 | 0.106 | 0.032 | 0.034 | 0.75 | 0.68 | 0.802 |
| 5329 | chr6 | 62995576 | 62996176 | 0.13 | 0.06 | 0.066 | 0.75 | 0.7 | 0.802 |
| 5330 | chr1 | 75602557 | 75603157 | 0.171 | 0.111 | 0.122 | 0.75 | 0.7 | 0.802 |
| 5331 | chr19 | 57182905 | 57183505 | 0.123 | 0.048 | 0.056 | 0.75 | 0.67 | 0.802 |
| 5332 | chr2 | 1.16E+08 | 1.16E+08 | 0.127 | 0.063 | 0.071 | 0.75 | 0.7 | 0.802 |
| 5333 | chr19 | 57182913 | 57183513 | 0.123 | 0.048 | 0.056 | 0.75 | 0.67 | 0.802 |
| 5334 | chr18 | 5196771 | 5197371 | 0.08 | 0.036 | 0.04 | 0.75 | 0.64 | 0.802 |
| 5335 | chr18 | 5196766 | 5197366 | 0.08 | 0.036 | 0.04 | 0.75 | 0.64 | 0.802 |
| 5336 | chr19 | 57182968 | 57183568 | 0.123 | 0.048 | 0.056 | 0.75 | 0.67 | 0.802 |
| 5337 | chr1 | 50513361 | 50513961 | 0.28 | 0.219 | 0.234 | 0.75 | 0.68 | 0.802 |
| 5338 | chr19 | 57182941 | 57183541 | 0.123 | 0.048 | 0.056 | 0.75 | 0.67 | 0.802 |
| 5339 | chr1 | 50513354 | 50513954 | 0.28 | 0.219 | 0.234 | 0.75 | 0.68 | 0.802 |
| 5340 | chr1 | 50513345 | 50513945 | 0.28 | 0.219 | 0.234 | 0.75 | 0.68 | 0.802 |
| 5341 | chr19 | 57182902 | 57183502 | 0.123 | 0.048 | 0.056 | 0.75 | 0.67 | 0.802 |
| 5342 | chr5 | 63802112 | 63802712 | 0.092 | 0.049 | 0.054 | 0.75 | 0.67 | 0.802 |
| 5343 | chr11 | 14995470 | 14996070 | 0.066 | 0.029 | 0.033 | 0.75 | 0.67 | 0.802 |
| 5344 | chr6 | 62995722 | 62996322 | 0.113 | 0.053 | 0.059 | 0.75 | 0.67 | 0.801 |
| 5345 | chr7 | 35293459 | 35294059 | 0.106 | 0.047 | 0.049 | 0.75 | 0.72 | 0.801 |
| 5346 | chr5 | 59189167 | 59189767 | 0.106 | 0.052 | 0.06 | 0.75 | 0.67 | 0.801 |
| 5347 | chr19 | 58095218 | 58095818 | 0.092 | 0.039 | 0.043 | 0.75 | 0.64 | 0.801 |
| 5348 | chr14 | 52535875 | 52536475 | 0.104 | 0.048 | 0.054 | 0.75 | 0.7 | 0.801 |
| 5349 | chr14 | 52535847 | 52536447 | 0.104 | 0.048 | 0.054 | 0.75 | 0.7 | 0.801 |
| 5350 | chr19 | 57183042 | 57183642 | 0.124 | 0.048 | 0.056 | 0.75 | 0.67 | 0.801 |
| 5351 | chr3 | 1.92E+08 | 1.92E+08 | 0.081 | 0.028 | 0.031 | 0.75 | 0.72 | 0.801 |
| 5352 | chr19 | 57182716 | 57183316 | 0.126 | 0.057 | 0.065 | 0.75 | 0.69 | 0.801 |
| 5353 | chr7 | 35293453 | 35294053 | 0.106 | 0.047 | 0.049 | 0.75 | 0.72 | 0.801 |
| 5354 | chr14 | 52535649 | 52536249 | 0.104 | 0.047 | 0.052 | 0.75 | 0.69 | 0.801 |
| 5355 | chr17 | 50235665 | 50236265 | 0.122 | 0.051 | 0.056 | 0.75 | 0.66 | 0.801 |
| 5356 | chr6 | 62995830 | 62996430 | 0.113 | 0.053 | 0.059 | 0.75 | 0.67 | 0.801 |
| 5357 | chr19 | 58095359 | 58095959 | 0.092 | 0.039 | 0.043 | 0.75 | 0.64 | 0.801 |
| 5358 | chr7 | 35293237 | 35293837 | 0.105 | 0.047 | 0.05 | 0.75 | 0.7 | 0.801 |
| 5359 | chr19 | 58095281 | 58095881 | 0.092 | 0.039 | 0.043 | 0.75 | 0.64 | 0.801 |
| 5360 | chr6 | 62995819 | 62996419 | 0.113 | 0.053 | 0.059 | 0.75 | 0.67 | 0.801 |
| 5361 | chr14 | 52535653 | 52536253 | 0.104 | 0.047 | 0.052 | 0.75 | 0.69 | 0.801 |
| 5362 | chr14 | 52535664 | 52536264 | 0.104 | 0.047 | 0.052 | 0.75 | 0.69 | 0.801 |
| 5363 | chr19 | 58095288 | 58095888 | 0.092 | 0.039 | 0.043 | 0.75 | 0.64 | 0.801 |
| 5364 | chrX | 8699872 | 8700472 | 0.049 | 0.018 | 0.02 | 0.75 | 0.7 | 0.801 |
| 5365 | chr1 | 1.15E+08 | 1.15E+08 | 0.063 | 0.026 | 0.029 | 0.75 | 0.68 | 0.801 |
| 5366 | chr14 | 52535722 | 52536322 | 0.104 | 0.047 | 0.052 | 0.75 | 0.69 | 0.801 |
| 5367 | chr19 | 58095295 | 58095895 | 0.092 | 0.039 | 0.043 | 0.75 | 0.64 | 0.801 |
| 5368 | chr14 | 52535587 | 52536187 | 0.104 | 0.047 | 0.052 | 0.75 | 0.69 | 0.801 |
| 5369 | chr7 | 43152242 | 43152842 | 0.071 | 0.032 | 0.034 | 0.75 | 0.71 | 0.801 |
| 5370 | chr7 | 43152168 | 43152768 | 0.071 | 0.032 | 0.034 | 0.75 | 0.71 | 0.801 |
| 5371 | chr3 | 1.92E+08 | 1.92E+08 | 0.081 | 0.028 | 0.031 | 0.75 | 0.72 | 0.801 |
| 5372 | chr3 | 1.92E+08 | 1.92E+08 | 0.081 | 0.028 | 0.031 | 0.75 | 0.72 | 0.801 |
| 5373 | chr16 | 12995303 | 12995903 | 0.118 | 0.063 | 0.072 | 0.75 | 0.64 | 0.801 |
| 5374 | chr1 | 1.71E+08 | 1.71E+08 | 0.082 | 0.05 | 0.058 | 0.75 | 0.67 | 0.801 |
| 5375 | chr19 | 57182818 | 57183418 | 0.128 | 0.053 | 0.061 | 0.75 | 0.67 | 0.8 |
| 5376 | chr14 | 52733953 | 52734553 | 0.095 | 0.044 | 0.048 | 0.75 | 0.67 | 0.8 |
| 5377 | chr1 | 2.37E+08 | 2.37E+08 | 0.109 | 0.046 | 0.051 | 0.75 | 0.67 | 0.8 |
| 5378 | chr14 | 52733986 | 52734586 | 0.095 | 0.044 | 0.048 | 0.75 | 0.67 | 0.8 |
| 5379 | chr14 | 52733951 | 52734551 | 0.095 | 0.044 | 0.048 | 0.75 | 0.67 | 0.8 |
| 5380 | chr1 | 77333956 | 77334556 | 0.079 | 0.025 | 0.028 | 0.75 | 0.64 | 0.8 |
| 5381 | chr1 | 77333770 | 77334370 | 0.079 | 0.025 | 0.028 | 0.75 | 0.64 | 0.8 |
| 5382 | chr1 | 77333907 | 77334507 | 0.079 | 0.025 | 0.028 | 0.75 | 0.64 | 0.8 |
| 5383 | chr14 | 52734025 | 52734625 | 0.095 | 0.044 | 0.048 | 0.75 | 0.67 | 0.8 |
| 5384 | chr14 | 52734094 | 52734694 | 0.095 | 0.044 | 0.048 | 0.75 | 0.67 | 0.8 |
| 5385 | chr8 | 69243186 | 69243786 | 0.06 | 0.019 | 0.021 | 0.75 | 0.67 | 0.8 |
| 5386 | chr8 | 69243210 | 69243810 | 0.06 | 0.019 | 0.021 | 0.75 | 0.67 | 0.8 |
| 5387 | chr1 | 1.66E+08 | 1.66E+08 | 0.083 | 0.046 | 0.047 | 0.75 | 0.74 | 0.8 |
| 5388 | chr1 | 1.66E+08 | 1.66E+08 | 0.083 | 0.046 | 0.047 | 0.75 | 0.74 | 0.8 |
| 5389 | chr19 | 58095168 | 58095768 | 0.089 | 0.038 | 0.042 | 0.75 | 0.66 | 0.8 |
| 5390 | chr14 | 52734097 | 52734697 | 0.095 | 0.044 | 0.048 | 0.75 | 0.67 | 0.8 |
| 5391 | chr1 | 75601868 | 75602467 | 0.126 | 0.046 | 0.048 | 0.75 | 0.8 | 0.86 |
| 5392 | chr7 | 153583701 | 153584375 | 0.114 | 0.029 | 0.027 | 0.75 | 0.83 | 0.858 |
| 5393 | chr8 | 85095225 | 85095824 | 0.095 | 0.035 | 0.038 | 0.75 | 0.8 | 0.855 |
| 5394 | chr11 | 131780961 | 131781560 | 0.133 | 0.035 | 0.036 | 0.75 | 0.82 | 0.851 |
| 5395 | chr8 | 72755932 | 72756681 | 0.091 | 0.029 | 0.03 | 0.75 | 0.76 | 0.845 |
| 5396 | chr14 | 38724342 | 38725008 | 0.124 | 0.038 | 0.042 | 0.75 | 0.79 | 0.839 |
| 5397 | chr18 | 70533999 | 70534598 | 0.129 | 0.053 | 0.06 | 0.75 | 0.77 | 0.839 |
| 5398 | chr6 | 123317188 | 123317787 | 0.093 | 0.038 | 0.042 | 0.75 | 0.71 | 0.837 |
| 5399 | chr4 | 21950268 | 21950867 | 0.111 | 0.046 | 0.05 | 0.75 | 0.79 | 0.835 |
| 5400 | chr19 | 30865857 | 30866531 | 0.116 | 0.032 | 0.032 | 0.75 | 0.77 | 0.834 |
| 5401 | chr13 | 112716945 | 112717544 | 0.169 | 0.082 | 0.088 | 0.75 | 0.77 | 0.827 |
| 5402 | chr16 | 6069102 | 6069701 | 0.154 | 0.077 | 0.084 | 0.75 | 0.76 | 0.825 |
| 5403 | chr14 | 48143898 | 48144497 | 0.108 | 0.053 | 0.058 | 0.75 | 0.72 | 0.825 |
| 5404 | chr13 | 37247945 | 37248544 | 0.09 | 0.024 | 0.023 | 0.75 | 0.74 | 0.814 |
| 5405 | chr1 | 50513450 | 50514049 | 0.24 | 0.176 | 0.187 | 0.75 | 0.67 | 0.811 |
| 5406 | chr3 | 147108544 | 147109143 | 0.164 | 0.075 | 0.091 | 0.75 | 0.69 | 0.806 |
| 5407 | chr13 | 28366477 | 28367151 | 0.077 | 0.03 | 0.032 | 0.75 | 0.64 | 0.804 |
| 5408 | chr14 | 52535876 | 52536475 | 0.104 | 0.048 | 0.054 | 0.75 | 0.7 | 0.801 |
| 5409 | chr6 | 29521121 | 29521720 | 0.105 | 0.031 | 0.034 | 0.75 | 0.67 | 0.799 |
| 5410 | chr12 | 126675749 | 126676348 | 0.133 | 0.056 | 0.062 | 0.75 | 0.64 | 0.798 |
| 5411 | chr13 | 79170004 | 79170603 | 0.148 | 0.074 | 0.082 | 0.75 | 0.67 | 0.784 |
| 5412 | chr6 | 96463603 | 96464202 | 0.117 | 0.059 | 0.062 | 0.75 | 0.71 | 0.78 |
| 5413 | chr18 | 74962373 | 74962972 | 0.136 | 0.065 | 0.072 | 0.75 | 0.63 | 0.771 |
| 5414 | chr13 | 112711710 | 112712309 | 0.112 | 0.04 | 0.043 | 0.75 | 0.66 | 0.768 |
| 5415 | chr1 | 237205085 | 237205684 | 0.269 | 0.191 | 0.203 | 0.75 | 0.66 | 0.766 |
| 5416 | chr11 | 32008662 | 32009261 | 0.152 | 0.084 | 0.098 | 0.75 | 0.64 | 0.761 |
| 5417 | chr7 | 32467358 | 32467957 | 0.114 | 0.053 | 0.057 | 0.75 | 0.64 | 0.747 |
| 5418 | chr19 | 22444294 | 22444893 | 0.157 | 0.101 | 0.111 | 0.75 | 0.62 | 0.739 |

The predicting of the presence, absence, or development of cancer in an individual may particularly involve determining percent methylated reference for the panel of one or more CpGs. A threshold percent methylated reference value may be applied in order to stratify an individual as having or not having cancer, or of having a high or low risk of CIN3 and/or cancer development, preferably wherein the cancer is cervical or endometrial cancer, more preferably wherein the cancer is cervical cancer.

In any of the assays described herein, the step of determining the methylation status of the one or more CpGs in the panel may comprise determining each CpG within:
1. SEQ ID NO 5759 and/or SEQ ID NO 5703 and/or SEQ ID NO 5731, and wherein when the cancer index value is about 1.146 or more the individual is classified as having CIN3 and/or cervical cancer or as having a high risk of CIN3 and/or cervical cancer development, and wherein the sensitivity of the assay is at least 95.00%, the specificity of the assay is at least 75.00%, and the AUC is 0.96, preferably wherein the cancer index value is the percent methylated reference for sequence defined by SEQ ID NO 5759 and/or SEQ ID NO 5703 and/or SEQ ID NO 5731;
2. SEQ ID NO 5759 and/or SEQ ID NO 5703 and/or SEQ ID NO 5731, and wherein when the cancer index value is less than about 1.146 the individual is classified as not having CIN3 and/or cervical cancer or as having a low risk of CIN3 and/or cervical cancer development, and wherein the sensitivity of the assay is at least 95.00%, the specificity of the assay is at least 75.00%, and the AUC is 0.96, preferably wherein the cancer index value is the percent methylated reference for sequence defined by SEQ ID NO 5759 and/or SEQ ID NO 5703 and/or SEQ ID NO 5731;
3. SEQ ID NO 5760 and/or SEQ ID NO 5704 and/or SEQ ID NO 5732, and wherein when the cancer index value is about 0.473 or more the individual is classified as having CIN3 and/or cervical cancer or as having a high risk of CIN3 and/or cervical cancer development, and wherein the sensitivity of the assay is at least 75.00%, the specificity of the assay is at least 85.00%, and the AUC is 0.96, preferably wherein the cancer index value is the percent methylated reference for sequence defined by SEQ ID NO 5760 and/or SEQ ID NO 5704 and/or SEQ ID NO 5732;
4. SEQ ID NO 5760 and/or SEQ ID NO 5704 and/or SEQ ID NO 5732, and wherein when the cancer index value is less than about 0.473 the individual is classified as not having CIN3 and/or cervical cancer or as having a low risk of CIN3 and/or cervical cancer development, and wherein the sensitivity of the assay is at least 75.00%, the specificity of the assay is at least 85.00%, and the AUC is 0.96, preferably wherein the cancer index value is the percent methylated reference for sequence defined by SEQ ID NO 5760 and/or SEQ ID NO 5704 and/or SEQ ID NO 5732;
5. SEQ ID NO 5761 and/or SEQ ID NO 5705 and/or SEQ ID NO 5733, and wherein when the cancer index value is about 1.546 or more the individual is classified as having CIN3 and/or cervical cancer or as having a high risk of CIN3 and/or cervical cancer development, and wherein the sensitivity of the assay is at least 95.00%, the specificity of the assay is at least 75.00%, and the AUC is 0.95, preferably wherein the cancer index value is the percent methylated reference for sequence defined by SEQ ID NO 5761 and/or SEQ ID NO 5705 and/or SEQ ID NO 5733;
6. SEQ ID NO 5761 and/or SEQ ID NO 5705 and/or SEQ ID NO 5733, and wherein when the cancer index value is less than about 1.546 the individual is classified as not having CIN3 and/or cervical cancer or as having a low risk of CIN3 and/or cervical cancer development, and wherein the sensitivity of the assay is at least 95.00%, the specificity of the assay is at least 75.00%, and the AUC is 0.95, preferably wherein the cancer index value is the percent methylated reference for sequence defined by SEQ ID NO 5761 and/or SEQ ID NO 5705 and/or SEQ ID NO 5733;
7. SEQ ID NO 5762 and/or SEQ ID NO 5706 and/or SEQ ID NO 5734, and wherein when the cancer index value is about 1.645 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5762 and/or SEQ ID NO 5706 and/or SEQ ID NO 5734;
8. SEQ ID NO 5762 and/or SEQ ID NO 5706 and/or SEQ ID NO 5734, and wherein when the cancer index value is less than about 1.645 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5762 and/or SEQ ID NO 5706 and/or SEQ ID NO 5734;
9. SEQ ID NO 5763 and/or SEQ ID NO 5707 and/or SEQ ID NO 5735, and wherein when the cancer index value is about 2.824 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.94, preferably wherein the cancer index value is the percent methylated reference for sequence defined by SEQ ID NO 5763 and/or SEQ ID NO 5707 and/or SEQ ID NO 5735;
10. SEQ ID NO 5763 and/or SEQ ID NO 5707 and/or SEQ ID NO 5735, and wherein when the cancer index value is less than about 2.824 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.94, preferably wherein the cancer index value is the percent methylated reference for sequence defined by SEQ ID NO 5763 and/or SEQ ID NO 5707 and/or SEQ ID NO 5735;
11. SEQ ID NO 5764 and/or SEQ ID NO 5708 and/or SEQ ID NO 5736, and wherein when the cancer index value is about 2.968 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5764 and/or SEQ ID NO 5708 and/or SEQ ID NO 5736;
12. SEQ ID NO 5764 and/or SEQ ID NO 5708 and/or SEQ ID NO 5736, and wherein when the cancer index value is less than about 2.968 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.94, preferably wherein the cancer index value is the percent methylated reference for sequence defined by SEQ ID NO 5764 and/or SEQ ID NO 5708 and/or SEQ ID NO 5736;
13. SEQ ID NO 5765 and/or SEQ ID NO 5709 and/or SEQ ID NO 5737, and wherein when the cancer index value is about 0.184 or more the individual is classified as having CIN3 and/or cervical cancer or as having a high risk of CIN3 and/or cervical cancer development, and wherein the sensitivity of the assay is at least 75.00%, the specificity of the assay is at least 80.00%, and the AUC is 0.93, preferably wherein the cancer index value is the percent methylated reference for sequence defined by SEQ ID NO 5765 and/or SEQ ID NO 5709 and/or SEQ ID NO 5737;
14. SEQ ID NO 5765 and/or SEQ ID NO 5709 and/or SEQ ID NO 5737, and wherein when the cancer index value is less than about 0.184 the individual is classified as not having CIN3 and/or cervical cancer or as having a low risk of CIN3 and/or cervical cancer development, and wherein the sensitivity of the assay is at least 93.00%, the specificity of the assay is at least 80.00%, and the AUC is 0.75, preferably wherein the cancer index value is the percent methylated reference for sequence defined by SEQ ID NO 5765 and/or SEQ ID NO 5709 and/or SEQ ID NO 5737;
15. SEQ ID NO 5766 and/or SEQ ID NO 5710 and/or SEQ ID NO 5738, and wherein when the cancer index value is about 0.716 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.93, preferably wherein the cancer index value is the percent methylated reference for sequence defined by SEQ ID NO 5766 and/or SEQ ID NO 5710 and/or SEQ ID NO 5738;
16. SEQ ID NO 5766 and/or SEQ ID NO 5710 and/or SEQ ID NO 5738, and wherein when the cancer index value is less than about 0.716 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.93, preferably wherein the cancer index value is the percent methylated reference for sequence defined by SEQ ID NO 5766 and/or SEQ ID NO 5710 and/or SEQ ID NO 5738;
17. SEQ ID NO 5767 and/or SEQ ID NO 5711 and/or SEQ ID NO 5739, and wherein when the cancer index value is about 3.482 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.93, preferably wherein the cancer index value is the percent methylated reference for sequence defined by SEQ ID NO 5767 and/or SEQ ID NO 5711 and/or SEQ ID NO 5739;
18. SEQ ID NO 5767 and/or SEQ ID NO 5711 and/or SEQ ID NO 5739, and wherein when the cancer index value is less than about 3.482 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.93, preferably wherein the cancer index value is the percent methylated reference for sequence defined by SEQ ID NO 5767 and/or SEQ ID NO 5711 and/or SEQ ID NO 5739;
19. SEQ ID NO 5768 and/or SEQ ID NO 5712 and/or SEQ ID NO 5740, and wherein when the cancer index value is about 3.818 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5768 and/or SEQ ID NO 5712 and/or SEQ ID NO 5740;
20. SEQ ID NO 5768 and/or SEQ ID NO 5712 and/or SEQ ID NO 5740, and wherein when the cancer index value is less than about 3.818 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5768 and/or SEQ ID NO 5712 and/or SEQ ID NO 5740;
21. SEQ ID NO 5769 and/or SEQ ID NO 5713 and/or SEQ ID NO 5741, and wherein when the cancer index value is about 0.295 or more the individual is classified as having CIN3 and/or cervical cancer or as having a high risk of CIN3 and/or cervical cancer development, and wherein the sensitivity of the assay is at least 80.00%, the specificity of the assay is at least 75.00%, and the AUC is 0.92, preferably wherein the cancer index value is the percent methylated reference for sequence defined by SEQ ID NO 5769 and/or SEQ ID NO 5713 and/or SEQ ID NO 5741;
22. SEQ ID NO 5769 and/or SEQ ID NO 5713 and/or SEQ ID NO 5741, and wherein when the cancer index value is less than about 0.295 the individual is classified as not having CIN3 and/or cervical cancer or as having a low risk of CIN3 and/or cervical cancer development, and wherein the sensitivity of the assay is at least 80.00%, the specificity of the assay is at least 75.00%, and the AUC is 0.92, preferably wherein the cancer index value is the percent methylated reference for sequence defined by SEQ ID NO 5769 and/or SEQ ID NO 5713 and/or SEQ ID NO 5741;
23. SEQ ID NO 5770 and/or SEQ ID NO 5714 and/or SEQ ID NO 5742, and wherein when the cancer index value is about 1.725 or more the individual is classified as having CIN3 and/or cervical cancer or as having a high risk of CIN3 and/or cervical cancer development, and wherein the sensitivity of the assay is at least 75.00%, the specificity of the assay is at least 75.00%, and the AUC is 0.92, preferably wherein the cancer index value is the percent methylated reference for sequence defined by SEQ ID NO 5770 and/or SEQ ID NO 5714 and/or SEQ ID NO 5742;
24. SEQ ID NO 5770 and/or SEQ ID NO 5714 and/or SEQ ID NO 5742, and wherein when the cancer index value is less than about 1.725 the individual is classified as not having CIN3 and/or cervical cancer or as having a low risk of CIN3 and/or cervical cancer development, and wherein the sensitivity of the assay is at least 75.00%, the specificity of the assay is at least 75.00%, and the AUC is 0.92, preferably wherein the cancer index value is the percent methylated reference for sequence defined by SEQ ID NO 5770 and/or SEQ ID NO 5714 and/or SEQ ID NO 5742;
25. SEQ ID NO 5771 and/or SEQ ID NO 5715 and/or SEQ ID NO 5743, and wherein when the cancer index value is about 3.258 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5771 and/or SEQ ID NO 5715 and/or SEQ ID NO 5743;
26. SEQ ID NO 5771 and/or SEQ ID NO 5715 and/or SEQ ID NO 5743, and wherein when the cancer index value is less than about 3.258 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5771 and/or SEQ ID NO 5715 and/or SEQ ID NO 5743;
27. SEQ ID NO 5772 and/or SEQ ID NO 5716 and/or SEQ ID NO 5744, and wherein when the cancer index value is about 4.731 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5772 and/or SEQ ID NO 5716 and/or SEQ ID NO 5744;
28. SEQ ID NO 5772 and/or SEQ ID NO 5716 and/or SEQ ID NO 5744, and wherein when the cancer index value is less than about 4.731 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5772 and/or SEQ ID NO 5716 and/or SEQ ID NO 5744;
29. SEQ ID NO 5773 and/or SEQ ID NO 5717 and/or SEQ ID NO 5745, and wherein when the cancer index value is about 1.427 or more the individual is classified as having CIN3 and/or cervical cancer or as having a high risk of CIN3 and/or cervical cancer development, and wherein the sensitivity of the assay is at least 80.00%, the specificity of the assay is at least 75.00%, and the AUC is 0.92, preferably wherein the cancer index value is the percent methylated reference for sequence defined by SEQ ID NO 5773 and/or SEQ ID NO 5717 and/or SEQ ID NO 5745;
30. SEQ ID NO 5773 and/or SEQ ID NO 5717 and/or SEQ ID NO 5745, and wherein when the cancer index value is less than about 1.427 the individual is classified as not having CIN3 and/or cervical cancer or as having a low risk of CIN3 and/or cervical cancer development, and wherein the sensitivity of the assay is at least 80.00%, the specificity of the assay is at least 75.00%, and the AUC is 0.92, preferably wherein the cancer index value is the percent methylated reference for sequence defined by SEQ ID NO 5773 and/or SEQ ID NO 5717 and/or SEQ ID NO 5745;
31. SEQ ID NO 5774 and/or SEQ ID NO 5718 and/or SEQ ID NO 5746, and wherein when the cancer index value is about 4.511 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5774 and/or SEQ ID NO 5718 and/or SEQ ID NO 5746;
32. SEQ ID NO 5774 and/or SEQ ID NO 5718 and/or SEQ ID NO 5746, and wherein when the cancer index value is less than about 4.511 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5774 and/or SEQ ID NO 5718 and/or SEQ ID NO 5746;
33. SEQ ID NO 5775 and/or SEQ ID NO 5719 and/or SEQ ID NO 5747, and wherein when the cancer index value is about 6.894 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5775 and/or SEQ ID NO 5719 and/or SEQ ID NO 5747;
34. SEQ ID NO 5775 and/or SEQ ID NO 5719 and/or SEQ ID NO 5747, and wherein when the cancer index value is less than about 6.894 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.92, preferably wherein the cancer index value is the percent methylated reference for sequence defined by SEQ ID NO 5775 and/or SEQ ID NO 5719 and/or SEQ ID NO 5747;
35. SEQ ID NO 5776 and/or SEQ ID NO 5720 and/or SEQ ID NO 5748, and wherein when the cancer index value is about 3.934 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5776 and/or SEQ ID NO 5720 and/or SEQ ID NO 5748;
36. SEQ ID NO 5776 and/or SEQ ID NO 5720 and/or SEQ ID NO 5748, and wherein when the cancer index value is less than about 3.934 or more the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5776 and/or SEQ ID NO 5720 and/or SEQ ID NO 5748;
37. SEQ ID NO 5777 and/or SEQ ID NO 5721 and/or SEQ ID NO 5749, and wherein when the cancer index value is about 4.153 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5777 and/or SEQ ID NO 5721 and/or SEQ ID NO 5749;
38. SEQ ID NO 5777 and/or SEQ ID NO 5721 and/or SEQ ID NO 5749, and wherein when the cancer index value is less than about 4.153 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5777 and/or SEQ ID NO 5721 and/or SEQ ID NO 5749;
39. SEQ ID NO 5778 and/or SEQ ID NO 5722 and/or SEQ ID NO 5750, and wherein when the cancer index value is about 0.468 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.91, preferably wherein the cancer index value is the percent methylated reference for sequence defined by SEQ ID NO 5778 and/or SEQ ID NO 5722 and/or SEQ ID NO 5750;
40. SEQ ID NO 5778 and/or SEQ ID NO 5722 and/or SEQ ID NO 5750, and wherein when the cancer index value is less than about 0.468 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.91, preferably wherein the cancer index value is the percent methylated reference for sequence defined by SEQ ID NO 5778 and/or SEQ ID NO 5722 and/or SEQ ID NO 5750;
41. SEQ ID NO 5779 and/or SEQ ID NO 5723 and/or SEQ ID NO 5751, and wherein when the cancer index value is about 2.226 or more the individual is classified as having CIN3 and/or cervical cancer or as having a high risk of CIN3 and/or cervical cancer development, and wherein the sensitivity of the assay is at least 90.00%, the specificity of the assay is at least 75.00%, and the AUC is 0.90, preferably wherein the cancer index value is the percent methylated reference for sequence defined by SEQ ID NO 5779 and/or SEQ ID NO 5723 and/or SEQ ID NO 5751;
42. SEQ ID NO 5779 and/or SEQ ID NO 5723 and/or SEQ ID NO 5751, and wherein when the cancer index value is less than about 2.226 the individual is classified as not having CIN3 and/or cervical cancer or as having a low risk of CIN3 and/or cervical cancer development, and wherein the sensitivity of the assay is at least 90.00%, the specificity of the assay is at least 75.00%, and the AUC is 0.90, preferably wherein the cancer index value is the percent methylated reference for sequence defined by SEQ ID NO 5779 and/or SEQ ID NO 5723 and/or SEQ ID NO 5751;
43. SEQ ID NO 5780 and/or SEQ ID NO 5724 and/or SEQ ID NO 5752, and wherein when the cancer index value is about 6.942 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.90, preferably wherein the cancer index value is the percent methylated reference for sequence defined by SEQ ID NO 5780 and/or SEQ ID NO 5724 and/or SEQ ID NO 5752;
44. SEQ ID NO 5780 and/or SEQ ID NO 5724 and/or SEQ ID NO 5752, and wherein when the cancer index value is less than about 6.942 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.90, preferably wherein the cancer index value is the percent methylated reference for sequence defined by SEQ ID NO 5780 and/or SEQ ID NO 5724 and/or SEQ ID NO 5752;
45. SEQ ID NO 5781 and/or SEQ ID NO 5725 and/or SEQ ID NO 5753, and wherein when the cancer index value is about 1.955 or more the individual is classified as having CIN3 and/or cervical cancer or as having a high risk of CIN3 and/or cervical cancer development, and wherein the sensitivity of the assay is at least 80.00%, the specificity of the assay is at least 75.00%, and the AUC is 0.90, preferably wherein the cancer index value is the percent methylated reference for sequence defined by SEQ ID NO 5781 and/or SEQ ID NO 5725 and/or SEQ ID NO 5753;
46. SEQ ID NO 5781 and/or SEQ ID NO 5725 and/or SEQ ID NO 5753, and wherein when the cancer index value is less than about 1.955 the individual is classified as not having CIN3 and/or cervical cancer or as having a low risk of CIN3 and/or cervical cancer development, and wherein the sensitivity of the assay is at least 80.00%, the specificity of the assay is at least 75.00%, and the AUC is 0.90, preferably wherein the cancer index value is the percent methylated reference for sequence defined by SEQ ID NO 5781 and/or SEQ ID NO 5725 and/or SEQ ID NO 5753;
47. SEQ ID NO 5782 and/or SEQ ID NO 5726 and/or SEQ ID NO 5754, and wherein when the cancer index value is about 5.005 or more the individual is classified as having CIN3 and/or cervical cancer or as having a high risk of CIN3 and/or cervical cancer development, and wherein the sensitivity of the assay is at least 75.00%, the specificity of the assay is at least 75.00%, and the AUC is 0.88, preferably wherein the cancer index value is the percent methylated reference for sequence defined by SEQ ID NO 5782 and/or SEQ ID NO 5726 and/or SEQ ID NO 5754;
48. SEQ ID NO 5782 and/or SEQ ID NO 5726 and/or SEQ ID NO 5754, and wherein when the cancer index value is less than about 5.005 the individual is classified as not having CIN3 and/or cervical cancer or as having a low risk of CIN3 and/or cervical cancer development, and wherein the sensitivity of the assay is at least 75.00%, the specificity of the assay is at least 75.00%, and the AUC is 0.88, preferably wherein the cancer index value is the percent methylated reference for sequence defined by SEQ ID NO 5782 and/or SEQ ID NO 5726 and/or SEQ ID NO 5754;
49. SEQ ID NO 5783 and/or SEQ ID NO 5727 and/or SEQ ID NO 5755, and wherein when the cancer index value is about 5.315 or more the individual is classified as having CIN3 and/or cervical cancer or as having a high risk of CIN3 and/or cervical cancer development, and wherein the sensitivity of the assay is at least 80.00%, the specificity of the assay is at least 75.00%, and the AUC is 0.86, preferably wherein the cancer index value is the percent methylated reference for sequence defined by SEQ ID NO 5783 and/or SEQ ID NO 5727 and/or SEQ ID NO 5755;
50. SEQ ID NO 5783 and/or SEQ ID NO 5727 and/or SEQ ID NO 5755, and wherein when the cancer index value is less than about 5.315 the individual is classified as not having CIN3 and/or cervical cancer or as having a low risk of CIN3 and/or cervical cancer development, and wherein the sensitivity of the assay is at least 80.00%, the specificity of the assay is at least 75.00%, and the AUC is 0.86, preferably wherein the cancer index value is the percent methylated reference for sequence defined by SEQ ID NO 5783 and/or SEQ ID NO 5727 and/or SEQ ID NO 5755;
51. SEQ ID NO 5784 and/or SEQ ID NO 5728 and/or SEQ ID NO 5756, and wherein when the cancer index value is about 5.381 or more the individual is classified as having CIN3 and/or cervical cancer or as having a high risk of CIN3 and/or cervical cancer development, and wherein the sensitivity of the assay is at least 70.00%, the specificity of the assay is at least 75.00%, and the AUC is 0.85, preferably wherein the cancer index value is the percent methylated reference for sequence defined by SEQ ID NO 5784 and/or SEQ ID NO 5728 and/or SEQ ID NO 5756;
52. SEQ ID NO 5784 and/or SEQ ID NO 5728 and/or SEQ ID NO 5756, and wherein when the cancer index value is less than about 5.381 the individual is classified as not having CIN3 and/or cervical cancer or as having a low risk of CIN3 and/or cervical cancer development, and wherein the sensitivity of the assay is at least 70.00%, the specificity of the assay is at least 75.00%, and the AUC is 0.85, preferably wherein the cancer index value is the percent methylated reference for sequence defined by SEQ ID NO 5784 and/or SEQ ID NO 5728 and/or SEQ ID NO 5756;
53. SEQ ID NO 5785 and/or SEQ ID NO 5729 and/or SEQ ID NO 5757, and wherein when the cancer index value is about 3.671 or more the individual is classified as having CIN3 and/or cervical cancer or as having a high risk of CIN3 and/or cervical cancer development, and wherein the sensitivity of the assay is at least 85.00%, the specificity of the assay is at least 75.00%, and the AUC is 0.84, preferably wherein the cancer index value is the percent methylated reference for sequence defined by SEQ ID NO 5785 and/or SEQ ID NO 5729 and/or SEQ ID NO 5757;
54. SEQ ID NO 5785 and/or SEQ ID NO 5729 and/or SEQ ID NO 5757, and wherein when the cancer index value is less than about 3.671 the individual is classified as not having CIN3 and/or cervical cancer or as having a low risk of CIN3 and/or cervical cancer development, and wherein the sensitivity of the assay is at least 85.00%, the specificity of the assay is at least 75.00%, and the AUC is 0.84, preferably wherein the cancer index value is the percent methylated reference for sequence defined by SEQ ID NO 5785 and/or SEQ ID NO 5729 and/or SEQ ID NO 5757;
55. SEQ ID NO 5786 and/or SEQ ID NO 5730 and/or SEQ ID NO 5758, and wherein when the cancer index value is about 0.652 or more the individual is classified as having CIN3 and/or cervical cancer or as having a high risk of CIN3 and/or cervical cancer development, and wherein the sensitivity of the assay is at least 55.00%, the specificity of the assay is at least 75.00%, and the AUC is 0.82, preferably wherein the cancer index value is the percent methylated reference for sequence defined by SEQ ID NO 5786 and/or SEQ ID NO 5730 and/or SEQ ID NO 5758;
56. SEQ ID NO 5786 and/or SEQ ID NO 5730 and/or SEQ ID NO 5758, and wherein when the cancer index value is less than about 0.652 the individual is classified as not having CIN3 and/or cervical cancer or as having a low risk of CIN3 and/or cervical cancer development, and wherein the sensitivity of the assay is at least 55.00%, the specificity of the assay is at least 75.00%, and the AUC is 0.82, preferably wherein the cancer index value is the percent methylated reference for sequence defined by SEQ ID NO 5786 and/or SEQ ID NO 5730 and/or SEQ ID NO 5758
57. SEQ ID NO 5759 and/or SEQ ID NO 5703 and/or SEQ ID NO 5731, and wherein when the cancer index value is about 0.003 or more the individual is classified as having CIN3 and/or cervical cancer or as having a high risk of CIN3 and/or cervical cancer development, and wherein the sensitivity of the assay is at least 86.00%, the specificity of the assay is at least 75.00%, and the AUC is 0.95, preferably wherein the cancer index value is the percent methylated reference for sequence defined by SEQ ID NO 5759 and/or SEQ ID NO 5703 and/or SEQ ID NO 5731;
58. SEQ ID NO 5759 and/or SEQ ID NO 5703 and/or SEQ ID NO 5731, and wherein when the cancer index value is less than about 0.003 the individual is classified as not having CIN3 and/or cervical cancer or as having a low risk of CIN3 and/or cervical cancer development, and wherein the sensitivity of the assay is at least 86.00%, the specificity of the assay is at least 75.00%, and the AUC is 0.95, preferably wherein the cancer index value is the percent methylated reference for sequence defined by SEQ ID NO 5759 and/or SEQ ID NO 5703 and/or SEQ ID NO 5731;
59. SEQ ID NO 5779 and/or SEQ ID NO 5723 and/or SEQ ID NO 5751, and wherein when the cancer index value is about 0.111 or more the individual is classified as having CIN3 and/or cervical cancer or as having a high risk of CIN3 and/or cervical cancer development, and wherein the sensitivity of the assay is at least 86.00%, the specificity of the assay is at least 75.00%, and the AUC is 0.90, preferably wherein the cancer index value is the percent methylated reference for sequence defined by SEQ ID NO 5779 and/or SEQ ID NO 5723 and/or SEQ ID NO 5751;
60. SEQ ID NO 5779 and/or SEQ ID NO 5723 and/or SEQ ID NO 5751, and wherein when the cancer index value is less than about 0.111 the individual is classified as not having CIN3 and/or cervical cancer or as having a low risk of CIN3 and/or cervical cancer development, and wherein the sensitivity of the assay is at least 86.00%, the specificity of the assay is at least 75.00%, and the AUC is 0.90, preferably wherein the cancer index value is the percent methylated reference for sequence defined by SEQ ID NO 5779 and/or SEQ ID NO 5723 and/or SEQ ID NO 5751;
61. SEQ ID NO 5769 and/or SEQ ID NO 5713 and/or SEQ ID NO 5741, and wherein when the cancer index value is about 0.022 or more the individual is classified as having CIN3 and/or cervical cancer or as having a high risk of CIN3 and/or cervical cancer development, and wherein the sensitivity of the assay is at least 75.00%, the specificity of the assay is at least 78.00%, and the AUC is 0.93, preferably wherein the cancer index value is the percent methylated reference for sequence defined by SEQ ID NO 5769 and/or SEQ ID NO 5713 and/or SEQ ID NO 5741;
62. SEQ ID NO 5769 and/or SEQ ID NO 5713 and/or SEQ ID NO 5741, and wherein when the cancer index value is less than about 0.022 the individual is classified as not having CIN3 and/or cervical cancer or as having a low risk of CIN3 and/or cervical cancer development, and wherein the sensitivity of the assay is at least 75.00%, the specificity of the assay is at least 78.00%, and the AUC is 0.93, preferably wherein the cancer index value is the percent methylated reference for sequence defined by SEQ ID NO 5769 and/or SEQ ID NO 5713 and/or SEQ ID NO 5741;
63. SEQ ID NO 5762 and/or SEQ ID NO 5706 and/or SEQ ID NO 5734, and wherein when the cancer index value is about 0.033 or more the individual is classified as having CIN3 and/or cervical cancer or as having a high risk of CIN3 and/or cervical cancer development, and wherein the sensitivity of the assay is at least 88.00%, the specificity of the assay is at least 75.00%, and the AUC is 0.93, preferably wherein the cancer index value is the percent methylated reference for sequence defined by SEQ ID NO 5762 and/or SEQ ID NO 5706 and/or SEQ ID NO 5734;
64. SEQ ID NO 5762 and/or SEQ ID NO 5706 and/or SEQ ID NO 5734, and wherein when the cancer index value is less than about 0.033 the individual is classified as not having CIN3 and/or cervical cancer or as having a low risk of CIN3 and/or cervical cancer development, and wherein the sensitivity of the assay is at least 88.00%, the specificity of the assay is at least 75.00%, and the AUC is 0.93, preferably wherein the cancer index value is the percent methylated reference for sequence defined by SEQ ID NO 5762 and/or SEQ ID NO 5706 and/or SEQ ID NO 5734;
65. SEQ ID NO 5761 and/or SEQ ID NO 5705 and/or SEQ ID NO 5733, and wherein when the cancer index value is about 0.001 or more the individual is classified as having CIN3 and/or cervical cancer or as having a high risk of CIN3 and/or cervical cancer development, and wherein the sensitivity of the assay is at least 84.00%, the specificity of the assay is at least 75.00%, and the AUC is 0.94, preferably wherein the cancer index value is the percent methylated reference for sequence defined by SEQ ID NO 5761 and/or SEQ ID NO 5705 and/or SEQ ID NO 5733;
66. SEQ ID NO 5761 and/or SEQ ID NO 5705 and/or SEQ ID NO 5733, and wherein when the cancer index value is less than about 0.001 the individual is classified as not having CIN3 and/or cervical cancer or as having a low risk of CIN3 and/or cervical cancer development, and wherein the sensitivity of the assay is at least 84.00%, the specificity of the assay is at least 75.00%, and the AUC is 0.94, preferably wherein the cancer index value is the percent methylated reference for sequence defined by SEQ ID NO 5761 and/or SEQ ID NO 5705 and/or SEQ ID NO 5733;
67. SEQ ID NO 5759 and/or SEQ ID NO 5703 and/or SEQ ID NO 5731, and wherein when the cancer index value is about 0.003 or more the individual is classified as having endometrial cancer or as having a high risk of endometrial cancer development, and wherein the sensitivity of the assay is at least 88.00%, the specificity of the assay is at least 75.00%, and the AUC is 0.95, preferably wherein the cancer index value is the percent methylated reference for sequence defined by SEQ ID NO 5759 and/or SEQ ID NO 5703 and/or SEQ ID NO 5731;
68. SEQ ID NO 5759 and/or SEQ ID NO 5703 and/or SEQ ID NO 5731, and wherein when the cancer index value is less than about 0.003 the individual is classified as not having endometrial cancer or as having a low risk of endometrial cancer development, and wherein the sensitivity of the assay is at least 88.00%, the specificity of the assay is at least 75.00%, and the AUC is 0.95, preferably wherein the cancer index value is the percent methylated reference for sequence defined by SEQ ID NO 5759 and/or SEQ ID NO 5703 and/or SEQ ID NO 5731;
69. SEQ ID NO 5779 and/or SEQ ID NO 5723 and/or SEQ ID NO 5751, and wherein when the cancer index value is about 0.111 or more the individual is classified as having endometrial cancer or as having a high risk of endometrial cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5779 and/or SEQ ID NO 5723 and/or SEQ ID NO 5751;
70. SEQ ID NO 5779 and/or SEQ ID NO 5723 and/or SEQ ID NO 5751, and wherein when the cancer index value is less than about 0.111 the individual is classified as not having endometrial cancer or as having a low risk of endometrial cancer development, and wherein the sensitivity of the assay is at least 85.00%, the specificity of the assay is at least 75.00%, and the AUC is 0.92, preferably wherein the cancer index value is the percent methylated reference for sequence defined by SEQ ID NO 5779 and/or SEQ ID NO 5723 and/or SEQ ID NO 5751;
71. SEQ ID NO 5769 and/or SEQ ID NO 5713 and/or SEQ ID NO 5741, and wherein when the cancer index value is about 0.022 or more the individual is classified as having endometrial cancer or as having a high risk of endometrial cancer development, and wherein the sensitivity of the assay is at least 72.00%, the specificity of the assay is at least 78.00%, and the AUC is 0.91, preferably wherein the cancer index value is the percent methylated reference for sequence defined by SEQ ID NO 5769 and/or SEQ ID NO 5713 and/or SEQ ID NO 5741;
72. SEQ ID NO 5769 and/or SEQ ID NO 5713 and/or SEQ ID NO 5741, and wherein when the cancer index value is less than about 0.022 the individual is classified as not having endometrial cancer or as having a low risk of endometrial cancer development, and wherein the sensitivity of the assay is at least 72.00%, the specificity of the assay is at least 78.00%, and the AUC is 0.91, preferably wherein the cancer index value is the percent methylated reference for sequence defined by SEQ ID NO 5769 and/or SEQ ID NO 5713 and/or SEQ ID NO 5741;
73. SEQ ID NO 5762 and/or SEQ ID NO 5706 and/or SEQ ID NO 5734, and wherein when the cancer index value is about 0.033 or more the individual is classified as having endometrial cancer or as having a high risk of endometrial cancer development, and wherein the sensitivity of the assay is at least 72.00%, the specificity of the assay is at least 75.00%, and the AUC is 0.85, preferably wherein the cancer index value is the percent methylated reference for sequence defined by SEQ ID NO 5762 and/or SEQ ID NO 5706 and/or SEQ ID NO 5734;
74. SEQ ID NO 5762 and/or SEQ ID NO 5706 and/or SEQ ID NO 5734, and wherein when the cancer index value is less than about 0.033 the individual is classified as not having endometrial cancer or as having a low risk of endometrial cancer development, and wherein the sensitivity of the assay is at least 72.00%, the specificity of the assay is at least 75.00%, and the AUC is 0.85, preferably wherein the cancer index value is the percent methylated reference for sequence defined by SEQ ID NO 5762 and/or SEQ ID NO 5706 and/or SEQ ID NO 5734;
75. SEQ ID NO 5761 and/or SEQ ID NO 5705 and/or SEQ ID NO 5733, and wherein when the cancer index value is about 0.001 or more the individual is classified as having endometrial cancer or as having a high risk of endometrial cancer development, and wherein the sensitivity of the assay is at least 80.00%, the specificity of the assay is at least 75.00%, and the AUC is 0.89, preferably wherein the cancer index value is the percent methylated reference for sequence defined by SEQ ID NO 5761 and/or SEQ ID NO 5705 and/or SEQ ID NO 5733; and/or
76. SEQ ID NO 5761 and/or SEQ ID NO 5705 and/or SEQ ID NO 5733, and wherein when the cancer index value is less than about 0.001 the individual is classified as not having endometrial cancer or as having a low risk of endometrial cancer development, and wherein the sensitivity of the assay is at least 80.00%, the specificity of the assay is at least 75.00%, and the AUC is 0.89, preferably wherein the cancer index value is the percent methylated reference for sequence defined by SEQ ID NO 5761 and/or SEQ ID NO 5705 and/or SEQ ID NO 5733.

In any of the described assays, the methylation status of the one or more CpGs in the panel is preferably determined by a percent methylated reference analysis, and the assay is for assessing the presence, absence or development of CIN3 and/or cancer, preferably the cancer is cervical cancer or endometrial cancer. Most preferably, the cancer is cervical cancer.

The ROC data set out in Tables 8, 10, 11 and 13 corresponding to each of SEQ ID NOs: 5703 to 5786 are derived by determining a cancer index value from a panel of CpGs, wherein the panel in each instance comprises all of the CpGs in the sequence(s) defined by the SEQ ID NO. The ROC data set out in Tables 8 and 13 relate to the performance of the assay of the invention in stratifying an individual as having or not having CIN3, or as having a high or low risk of developing CIN3. The ROC data set out in Table 11 relates to the performance of the assay of the invention in stratifying an individual as having or not having endometrial cancer, or as having a high or low risk of developing endometrial cancer.

TABLE 8

| | | | | | | SEQ ID NO: |
|---|---|---|---|---|---|---|
| Gene | auc | cutoff | specificity | sensitivity | Probe (5′ 6-FAM-3′ BHQ-1) | of probe |
| DPP6 | 0.96 | 1.146 | 0.75 | 0.95 | CGTGCGTCGCGCGCGTA | 5759 |
| NTM | 0.96 | 0.473 | 0.85 | 0.75 | TTTTTCGGGCGAGTATAGGAAGTGGGTG | 5760 |
| LHX8 | 0.95 | 1.546 | 0.75 | 0.95 | TTCGGGTTTAGGCGTCGTGACGG | 5761 |
| RALYL | 0.94 | 1.645 | 0.75 | 0.85 | AGCGGTAGTTCGCGGCGAGGTT | 5762 |
| FUT9 | 0.94 | 2.824 | 0.75 | 0.9 | ATCGAGGTAGCGATAGCGGCGGG | 5763 |
| LINC00403 | 0.94 | 2.968 | 0.75 | 0.85 | TTGGTCGTGTCGTGGCGCG | 5764 |
| CLVS2 | 0.93 | 0.184 | 0.8 | 0.75 | CGGTTGTTGCGCGGCGTTG | 5765 |
| KCNIP4 | 0.93 | 0.716 | 0.75 | 0.9 | CGGGCGGTTCGAGTGCGG | 5766 |
| NET01 | 0.93 | 3.482 | 0.75 | 0.85 | ATTCGGCGACGGTTGATCGCG | 5767 |
| CLEC14A | 0.92 | 3.818 | 0.75 | 0.9 | TGTAGTTGGAAGGGCGCGCGATAG | 5768 |
| GSX1 | 0.92 | 0.295 | 0.75 | 0.8 | CGATCGCGCGTCGG | 5769 |
| ZNF536 | 0.92 | 1.725 | 0.75 | 0.75 | CGCGGTGACGGCGTTTTTAATTGA | 5770 |
| PDEIC | 0.92 | 3.258 | 0.75 | 0.9 | TTATCGTAGTTTGAGGCGTTATCGGTTGTTTTC | 5771 |
| MDGA | 0.92 | 4.731 | 0.75 | 0.85 | CGAGGCGAAGTGTTTTTTAGGGAAGCG | 5772 |
| MHC | 0.92 | 1.427 | 0.75 | 0.8 | TTGGTTAGCGTGCGATATAGGCGCG | 5773 |
| NID2 | 0.92 | 4.511 | 0.75 | 0.85 | AATTTCGTAGCGGCGAGTGCGGT | 5774 |
| ZIC4 | 0.92 | 6.894 | 0.75 | 0.9 | CGTTCGTCGTCGTTTAGTTTTGGTTACGATT | 5775 |
| RCN1 | 0.92 | 3.934 | 0.75 | 0.85 | CGTTTTTCGTCGACGTTATTTGTTTTGCG | 5776 |
| RYR2 | 0.92 | 4.153 | 0.75 | 0.85 | TCGGAGGAGCGGGTTGCGG | 5777 |
| MSC | 0.91 | 0.468 | 0.75 | 0.85 | TTCGCGGCGTAGAGCGTAGTTACGTT | 5778 |
| LINC00403 | 0.9 | 2.226 | 0.75 | 0.9 | TTGTCGGTGTTCGTTCGAGAGTTTTATTAGCG | 5779 |
| RBFOX | 0.9 | 6.942 | 0.75 | 0.85 | TCGGTGGGATTGGTTGCGTTGTTTT | 5780 |
| GALR1 | 0.9 | 1.955 | 0.75 | 0.8 | TGTTCGGTTTGATTTTCGCGTTGGG | 5781 |
| RNF291 | 0.88 | 5.005 | 0.75 | 0.75 | TTTTGGCGTGGATTCGATTGTGTCG | 5782 |
| REG39 | 0.86 | 5.315 | 0.75 | 0.8 | TAGTGGTTTTCGTGGGCGGTTTCGT | 5783 |
| RP4 | 0.85 | 5.381 | 0.75 | 0.7 | TTTTCGGAGCGTATTCGGTATTTTCGTTGTTT | 5784 |
| ELAVL4 | 0.84 | 3.671 | 0.75 | 0.85 | CGAGAGCGGTGAGATTTTGCGGA | 5785 |
| SERTM1 | 0.82 | 0.652 | 0.75 | 0.55 | CGGTAGGTTTTTTGCGTTAGCGGTGG | 5786 |

TABLE 13

| | | | | | | | SEQ ID | | | | | |
|---|---|---|---|---|---|---|---|---|---|---|---|---|
| | | | | | | | NO of | | | | | |
| | | | | | | | probe | | | | | |
| | | | | | SEQ ID | SEQ ID | (5′ 6- | | | | | |
| | | | | | NO of | NO of | FAM- | | | | | |
| | | | speci- | sensi- | forward | reverse | 3′ BHQ- | | | Amplicon | Amplicon | DNA |
| gene_name | auc | cutoff | ficity | tivity | primer | primer | 1) | ML.Design.Region | Chrm | location | size | strand |
| CLEC14A_i | 0.92 | 3.818 | 0.75 | 0.9 | 5712 | 5740 | 5768 | chr14:38724342-38725008 | 14 | chr14:38724596- | 83 | pos |
| | | | | | | | | | | 38724678 | | |
| CLVS2_ii | 0.93 | 0.184 | 0.8 | 0.75 | 5709 | 5737 | 5765 | chr6:123317188-123317787 | 6 | chr6:123317440- | 95 | neg |
| | | | | | | | | | | 123317534 | | |
| DPP6_i | 0.95 | 0.003 | 0.75 | 0.86 | 5703 | 5731 | 5759 | chr7:153583701-153584375 | 7 | chr7:153584008- | 72 | pos |
| | | | | | | | | | | 153584079 | | |
| ELAVL4_i | 0.84 | 3.671 | 0.75 | 0.85 | 5729 | 5757 | 5785 | chr1:50513450-50514049 | 1 | chr1:50513718- | 71 | pos |
| | | | | | | | | | | 50513788 | | |
| FUT9_ii | 0.94 | 2.824 | 0.75 | 0.9 | 5707 | 5735 | 5763 | chr6:96463603-96464202 | 6 | chr6:96463902- | 103 | neg |
| | | | | | | | | | | 96464004 | | |
| GALR1_i | 0.9 | 1.955 | 0.75 | 0.8 | 5725 | 5753 | 5781 | chr18:74962373-74962972 | 18 | chr18:74962595- | 90 | pos |
| | | | | | | | | | | 74962684 | | |
| GSXiii | 0.93 | 0.022 | 0.78 | 0.75 | 5713 | 5741 | 5769 | chr13:28366477-28367151 | 13 | chr13:28366785- | 82 | pos |
| | | | | | | | | | | 28366866 | | |
| KCNIP4_i | 0.93 | 0.716 | 0.75 | 0.9 | 5710 | 5738 | 5766 | chr4:21950268-21950867 | 4 | chr4:21950535- | 62 | pos |
| | | | | | | | | | | 21950596 | | |
| LHX8_i | 0.94 | 0.001 | 0.75 | 0.84 | 5705 | 5733 | 5761 | chr1:75601868-75602467 | 1 | chr1:75602107- | 79 | pos |
| | | | | | | | | | | 75602185 | | |
| LINC00403_i | 0.9 | 0.111 | 0.75 | 0.86 | 5723 | 5751 | 5779 | chr13:112711710-112712309 | 13 | chr13:112711967- | 86 | pos |
| | | | | | | | | | | 112712052 | | |
| LINC00403_iv | 0.94 | 2.968 | 0.75 | 0.85 | 5708 | 5736 | 5764 | chr13:112716945-112717544 | 13 | chr13:112717233- | 85 | neg |
| | | | | | | | | | | 112717317 | | |
| MDGA_I | 0.92 | 4.731 | 0.75 | 0.85 | 5716 | 5744 | 5772 | chr14:48143898-48144497 | 14 | chr14:48144084- | 115 | pos |
| | | | | | | | | | | 48144198 | | |
| MHC_ii | 0.92 | 1.427 | 0.75 | 0.8 | 5717 | 5745 | 5773 | chr6:29521121-29521720 | 6 | chr6:29521375- | 85 | neg |
| | | | | | | | | | | 29521459 | | |
| MSC_ii | 0.91 | 0.468 | 0.75 | 0.85 | 5722 | 5750 | 5778 | chr8:72755932-72756681 | 8 | chr8:72756267- | 82 | neg |
| | | | | | | | | | | 72756348 | | |
| NET01_i | 0.93 | 3.482 | 0.75 | 0.85 | 5711 | 5739 | 5767 | chr18:70533999-70534598 | 18 | chr18:70534272- | 75 | pos |
| | | | | | | | | | | 70534346 | | |
| NID2_i | 0.92 | 4.511 | 0.75 | 0.85 | 5718 | 5746 | 5774 | chr14:52535876-52536475 | 14 | chr14:52536129- | 70 | pos |
| | | | | | | | | | | 52536198 | | |
| NTM_ii | 0.96 | 0.473 | 0.85 | 0.75 | 5704 | 5732 | 5760 | chr11:131780961-131781560 | 11 | chr11:131781252- | 103 | neg |
| | | | | | | | | | | 131781354 | | |
| PDEIC_ii | 0.92 | 3.258 | 0.75 | 0.9 | 5715 | 5743 | 5771 | chr7:32467358-32467957 | 7 | chr7:32467624- | 81 | neg |
| | | | | | | | | | | 32467704 | | |
| RALYL_ii | 0.93 | 0.033 | 0.75 | 0.88 | 5706 | 5734 | 5762 | chr8:85095225-85095824 | 8 | chr8:85095492- | 89 | neg |
| | | | | | | | | | | 85095580 | | |
| RBFOX_i | 0.9 | 6.942 | 0.75 | 0.85 | 5724 | 5752 | 5780 | chr16:6069102-6069701 | 16 | chr16:6069328- | 76 | pos |
| | | | | | | | | | | 6069403 | | |
| RCN1_i | 0.92 | 3.934 | 0.75 | 0.85 | 5720 | 5748 | 5776 | chr11:32008662-32009261 | 11 | chr11:32008959- | 84 | pos |
| | | | | | | | | | | 32009042 | | |
| REG39_i | 0.86 | 5.315 | 0.75 | 0.8 | 5727 | 5755 | 5783 | chr19:22444294-22444893 | 19 | chr19:22444593- | 71 | pos |
| | | | | | | | | | | 22444663 | | |
| RNF291_ii | 0.88 | 5.005 | 0.75 | 0.75 | 5726 | 5754 | 5782 | chr13:79170004-79170603 | 13 | chr13:79170284- | 84 | neg |
| | | | | | | | | | | 79170367 | | |
| RP4_ii | 0.85 | 5.381 | 0.75 | 0.7 | 5728 | 5756 | 5784 | chr12:126675749-126676348 | 12 | chr12:126676038- | 102 | neg |
| | | | | | | | | | | 126676139 | | |
| RYR2_i | 0.92 | 4.153 | 0.75 | 0.85 | 5721 | 5749 | 5777 | chr1:237205085-237205684 | 1 | chr1:237205344- | 67 | pos |
| | | | | | | | | | | 237205410 | | |
| SERTM1_ii | 0.82 | 0.652 | 0.75 | 0.55 | 5730 | 5758 | 5786 | chr13:37247945-37248544 | 13 | chr13:37248238- | 84 | neg |
| | | | | | | | | | | 37248321 | | |
| ZIC4 | 0.92 | 6.894 | 0.75 | 0.9 | 5719 | 5747 | 5775 | chr3:147108544-147109143 | 3 | chr3:147108796- | 89 | neg |
| | | | | | | | | | | 147108884 | | |
| ZNF536_ii | 0.92 | 1.725 | 0.75 | 0.75 | 5714 | 5742 | 5770 | chr19:30865857-30866531 | 19 | chr19:30866195- | 85 | neg |
| | | | | | | | | | | 30866279 | | |

TABLE 11

| | | | | | SEQ ID NO | SEQ ID NO | SEQ ID NO |
|---|---|---|---|---|---|---|---|
| | | | | | of forward | of reverse | of probe |
| gene_name | auc | cutoff | specificity | sensitivity | primer | primer | (5′ 6-FAM-3′ BHQ-1) |
| DPP6_i | 0.95 | 0.003 | 0.75 | 0.88 | 5703 | 5731 | 5759 |
| LINC00403_i | 0.92 | 0.111 | 0.75 | 0.85 | 5723 | 5751 | 5779 |
| GSXiii | 0.91 | 0.022 | 0.78 | 0.72 | 5713 | 5741 | 5769 |
| RALYL_ii | 0.85 | 0.033 | 0.75 | 0.72 | 5706 | 5734 | 5762 |
| LHX8_i | 0.89 | 0.001 | 0.75 | 0.8 | 5705 | 5733 | 5761 |

### Relationship Between Cancer Index Value and Determining Methylation Status of CpGs

In view of the observations described herein (see Examples), the inventors derived a cancer index based on an analysis of methylation status (DNAme; as described above) for use in assays for assessing the presence or development of cancer in an individual.

As explained herein, the described assays particularly relate to the assessment of assessing the presence, absence or development of cervical cancer and/or endometrial cancer, particularly cervical cancer.

Any of the assays described herein involve deriving a cancer index value based on the methylation of status of a panel of one or more CpGs assayed in a sample provided from an individual, as described and defined herein.

The cancer index value may be derived by any suitable means.

The inventors have identified specific CpGs, as described and defined herein, which may be used to form a panel of CpGs whose methylation status is determined in order to establish cancer index values in accordance with the assays described and defined herein. Using these panels the inventors have demonstrated that it is possible to derive a cancer index value which correlates with and is indicative of normal tissue, i.e. tissue which is CIN3 and/or cancer negative, in particular cervical and/or endometrial tissue which is cancer negative. Accordingly, cancer can be assessed to be absent in the individual. Using these panels the inventors have demonstrated that it is possible to derive a cancer index value which correlates with and is indicative of CIN3 and/or cancer tissue, i.e. tissue which is CIN3 and/or cancer positive, in particular cervical and/or endometrial tissue which is cancer negative. Accordingly, CIN3 and/or cancer can be assessed to be present in the individual. As explained herein, the inventors have shown that using panels of the CpGs that have been identified. Accordingly, CIN3 and/or cancer can be assessed to be present in the individual. As explained herein, the inventors have shown that using panels of the CpGs that have been identified it can be shown that the DNA methylation profile of normal cells, particularly from epithelial cells, particularly derived from the cervix, vaginal, the buccal area, blood or urine, or from a cervical liquid-based cytology sample, and more preferably from a cervical smear sample, as indicated by the cancer index value, is dynamic and subject to change on a continuum from indicating CIN3 and/or cancer negative to CIN3 and/or cancer positive tissue. In particular, the cancer index value described herein acts as a surrogate for indicating whether the cervical and/or endometrial tissue of an individual is cancer negative or cancer positive to a high degree of statistical accuracy. As such, using panels of the CpGs that have been identified it is possible to establish a cancer index value scale that can be used to assess the presence, absence or development of CIN3 and/or cancer, particularly cervical or endometrial cancer, most preferably cervical cancer, in an individual.

As described herein, the inventors have used certain methods for determining the methylation status of specific CpGs in the population of DNA molecules in the sample. For example, in one method a percent methylated reference (PMR) value of a CpG may be determined. In another method the methylation β-values of a CpG may be determined. Different mechanisms may be employed to determine specific values depending on the circumstances, such as PCR-based mechanisms or array-based mechanisms.

As will be apparent to a skilled person, in the assays of the invention the steps of determining the methylation status of specific CpGs in the population of DNA molecules in the sample are not limited to any one specific methodology. As the skilled person will appreciate, because the cancer index value is based on the methylation status of CpGs, and since the methylation status of CpGs can be represented by values which may be specific to a specific methodology, e.g. percent methylated reference (PMR) value or methylation β-value, then the range of cancer index values which define cancer negative and cancer positive samples may be dependent upon the methodology used to determine the methylation status of CpGs. Nevertheless, a user may readily reproduce and implement the assays of the invention using any suitable methodology for determining the methylation status of CpGs, provided that the same methodology is used consistently. Moreover, the user can readily establish, de novo, cancer index values which define cancer negative and cancer positive samples by determining the methylation status of CpGs in panels constituting the specific CpGs disclosed herein from known cancer negative and cancer positive patient samples. Once such cancer index values are established using the CpGs identified herein, a user may use these values as a basis for assessing the presence, absence or development of CIN3 and/or cancer, particularly cervical or endometrial cancer, most preferably cervical cancer, in any test individual whose cancer status is to be determined. Accordingly, cancer index values according to the present invention are not limited to specific methods of determination of methylation status of CpGs. On the contrary, the skilled person will appreciate that cancer index values can be established which reflect the intrinsic capabilities of the CpGs identified herein to correlate methylation status with CIN3 and/or cancer disease status.

Accordingly, the cancer index value may be derived by assessing the methylation status of the one or more CpGs in the panel in a sample provided from an individual by any suitable means.

The step of determining the methylation status of each CpG in the panel of one or more CpGs may be achieved by determining a percent methylated reference (PMR) value of each one of the one or more CpGs. The step of determining the methylation status of each CpG in the panel of one or more CpGs may be achieved by determining the methylation β-value of each one of the one or more CpGs.

In any of the assays described herein, the methylation status of the CpGs may be determined by any suitable means. For example, in any of the assays described herein the step of determining the methylation status of each CpG in the panel of one or more CpGs may comprise:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product.

The step of determining the methylation status of each CpG in the panel of one or more CpGs may comprise a conversion step in order to distinguish methylated CpG dinucleotides relative to non-methylated CpG dinucleotides. The conversion step may comprise e.g. bisulfate conversion or TAPS (TET-assisted pyridine borane sequencing) conversion of the DNA in a sample that is to be applied to any one or more of a. to c. above. TAPS may particularly involve the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

The step of determining the methylation status of each CpG in the panel of one or more CpGs may additionally, or alternatively, comprise the use of TempO-seq (templated Olig-sequencing). The oligonucleotides in the context of TempO-seq may or may not be designed such that they hybridise with methylated CpG dinucleotides following a prior conversion as described herein.

The step of determining the methylation status of each CpG in the panel of one or more CpGs may comprise the contacting the DNA in the sample with one or more methylation sensitive restriction endonucleases that cleave methylated and/or unmethylated forms of their restriction sites, and preferably the contacting of the DNA is prior to performing any one of a. to c. above. In assays of the invention wherein methylation sensitive restriction enzymes are used, one or more control reactions are performed. Preferably, the one or more control reactions involve interrogation of known loci that contain (i) no restriction endonuclease sites; (ii) a restriction site that is methylated; (iii) a restriction site that is unmethylated.

Using any of the methods for determining the methylation status of each CpG in the panel of one or more CpGs, the proportion of methylated and unmethylated CpGs at any given locus may be determined, thereby enabling generation of a cancer index value.

Preferably, the step of determining in the population of DNA molecules in the sample the methylation status of a panel of one or more CpGs further comprises determining a β value of each CpG. Deriving the cancer index value may involve providing a methylation β-value data set comprising the methylation β-values for each CpG in the panel of one or more CpGs.

### Assessment of Methylation Status of CpGs

Methylation of DNA is a recognised form of epigenetic modification which has the capability of altering the expression of genes and other elements such as microRNAs. In cancer development and progression, methylation may have the effect of e.g. silencing tumor suppressor genes and/or increasing the expression of oncogenes. Other forms of dysregulation may occur as a result of methylation. Methylation of DNA occurs at discrete loci which are predominately dinucleotides consisting of a CpG motif, but may also occur at CHH motifs (where H is A, C, or T). During methylation, a methyl group is added to the fifth carbon of cytosine bases to create methylcytosine.

Methylation can occur throughout the genome and is not limited to regions with respect to an expressed sequence such as a gene. Methylation typically, but not always, occurs in a promoter or other regulatory region of an expressed sequence such as enhancer elements. Most typically, the methylation status of CpGs is clustered in CpG islands, for example CpG islands present in the regulatory regions of genes, especially in their promoter regions.

Typically, an assessment of DNA methylation status involves analysing the presence or absence of methyl groups in DNA, for example methyl groups on the 5 position of one or more cytosine nucleotides. Preferably, the methylation status of one or more cytosine nucleotides present as a CpG dinucleotide (where C stands for Cytosine, G for Guanine and p for the phosphate group linking the two) is assessed.

A variety of techniques are available for the identification and assessment of CpG methylation status, as will be outlined briefly below. The assays described herein encompass any suitable technique for the determination of CpG methylation status.

Methyl groups are lost from a starting DNA molecule during conventional in vitro handling steps such as PCR. To avoid this, techniques for the detection of methyl groups commonly involve the preliminary treatment of DNA prior to subsequent processing, in a way that preserves the methylation status information of the original DNA molecule. Such preliminary techniques involve three main categories of processing, i.e. bisulphite modification, restriction enzyme digestion and affinity-based analysis. Products of these techniques can then be coupled with sequencing or array-based platforms for subsequent identification or qualitative assessment of CpG methylation status.

Techniques involving bisulphite modification of DNA have become the most common assays for detection and assessment of methylation status of CpG dinucleotides. Treatment of DNA with bisulphite, e.g. sodium bisulphite, converts cytosine bases to uracil bases, but has no effect on 5-methylcytosines. Thus, the presence of a cytosine in bisulphite-treated DNA is indicative of the presence of a cytosine base which was previously methylated in the starting DNA molecule. Such cytosine bases can be detected by a variety of techniques. For example, primers specific for unmethylated versus methylated DNA can be generated and used for PCR-based identification of methylated CpG dinucleotides. DNA may be amplified, either before or after bisulphite conversion. A separation/capture step may be performed, e.g. using binding molecules such as complementary oligonucleotide sequences. Standard and next-generation DNA sequencing protocols can also be used.

In other approaches, methylation-sensitive enzymes can be employed which digest or cut only in the presence of methylated DNA. Analysis of resulting fragments is commonly carried out using microarrays.

Affinity-based techniques exploit binding interactions to capture fragments of methylated DNA for the purposes of enrichment. Binding molecules such as anti-5-methylcytosine antibodies are commonly employed prior to subsequent processing steps such as PCR and sequencing.

Olkhov-Mitsel and Bapat (2012) provide a comprehensive review of techniques available for the identification and assessment of biomarkers involving methylcytosine.

For the purposes of assessing the methylation status of the CpG-based biomarkers characterised and described herein, any suitable assay can be employed.

Assays described herein may comprise determining methylation status of CpGs by bisulphite converting the DNA. Preferred assays involve bisulphite treatment of DNA, including amplification of the identified CpG loci for methylation specific PCR and/or sequencing and/or assessment of the methylation status of target loci using methylation-discriminatory microarrays.

Amplification of CpG loci can be achieved by a variety of approaches. Preferably, CpG loci are amplified using PCR. A variety of PCR-based approaches may be used. For example, methylation-specific primers may be hybridized to DNA containing the CpG sequence of interest. Such primers may be designed to anneal to a sequence derived from either a methylated or non-methylated CpG locus. Following annealing, a PCR reaction is performed and the presence of a subsequent PCR product indicates the presence of an annealed CpG of identifiable sequence. In such assays, DNA is bisulphite converted prior to amplification. Such techniques are commonly referred to as methylation specific PCR (MSP).

In other techniques, PCR primers may anneal to the CpG sequence of interest independently of the methylation status, and further processing steps may be used to determine the status of the CpG. Assays are designed so that the CpG site(s) are located between primer annealing sites. This assay scheme is used in techniques such as bisulphite genomic sequencing, COBRA, Ms-SNuPE. In such assay, DNA can be bisulphite converted before or after amplification.

Small-scale PCR approaches may be used. Such approaches commonly involve mass partitioning of samples (e.g. digital PCR). These techniques offer robust accuracy and sensitivity in the context of a highly miniaturised system (pico-liter sized droplets), ideal for the subsequent handling of small quantities of DNA obtainable from the potentially small volume of cellular material present in biological samples, particularly urine samples. A variety of such small-scale PCR techniques are widely available. For example, microdroplet-based PCR instruments are available from a variety of suppliers, including RainDance Technologies, Inc. (Billerica, MA; http://raindancetech.com/) and Bio-Rad, Inc. (http://www.bio-rad.com/). Microarray platforms may also be used to carry out small-scale PCR. Such platforms may include microfluidic network-based arrays e.g. available from Fluidigm Corp. (www.fluidigm.com).

Following amplification of CpG loci, amplified PCR products may be coupled to subsequent analytical platforms in order to determine the methylation status of the CpGs of interest. For example, the PCR products may be directly sequenced to determine the presence or absence of a methylcytosine at the target CpG or analysed by array-based techniques.

Any suitable sequencing techniques may be employed to determine the sequence of target DNA. In the assays of the present invention the use of high-throughput, so-called “second generation”, “third generation” and “next generation” techniques to sequence bisulphite-treated DNA can be used.

In second generation techniques, large numbers of DNA molecules are sequenced in parallel. Typically, tens of thousands of molecules are anchored to a given location at high density and sequences are determined in a process dependent upon DNA synthesis. Reactions generally consist of successive reagent delivery and washing steps, e.g. to allow the incorporation of reversible labelled terminator bases, and scanning steps to determine the order of base incorporation. Array-based systems of this type are available commercially e.g. from Illumina, Inc. (San Diego, CA; http://www.illumina.com/).

Third generation techniques are typically defined by the absence of a requirement to halt the sequencing process between detection steps and can therefore be viewed as real-time systems. For example, the base-specific release of hydrogen ions, which occurs during the incorporation process, can be detected in the context of microwell systems (e.g. see the Ion Torrent system available from Life Technologies; http://www.lifetechnologies.com/). Similarly, in pyrosequencing the base-specific release of pyrophosphate (PPi) is detected and analysed. In nanopore technologies, DNA molecules are passed through or positioned next to nanopores, and the identities of individual bases are determined following movement of the DNA molecule relative to the nanopore. Systems of this type are available commercially e.g. from Oxford Nanopore (https://www.nanoporetech.com/). In an alternative assay, a DNA polymerase enzyme is confined in a “zero-mode waveguide” and the identity of incorporated bases are determined with florescence detection of gamma-labeled phosphonucleotides (see e.g. Pacific Biosciences; http://www.pacificbiosciences.com/).

In other assays sequencing steps may be omitted. For example, amplified PCR products may be applied directly to hybridization arrays based on the principle of the annealing of two complementary nucleic acid strands to form a double-stranded molecule. Hybridization arrays may be designed to include probes which are able to hybridize to amplification products of a CpG and allow discrimination between methylated and non-methylated loci. For example, probes may be designed which are able to selectively hybridize to an CpG locus containing thymine, indicating the generation of uracil following bisulphite conversion of an unmethylated cytosine in the starting template DNA. Conversely, probes may be designed which are able to selectively hybridize to a CpG locus containing cytosine, indicating the absence of uracil conversion following bisulphite treatment. This corresponds with a methylated CpG locus in the starting template DNA.

Following the application of a suitable detection system to the array, computer-based analytical techniques can be used to determine the methylation status of a CpG. Detection systems may include, e.g. the addition of fluorescent molecules following a methylation status-specific probe extension reaction. Such techniques allow CpG status determination without the specific need for the sequencing of CpG amplification products. Such array-based discriminatory probes may be termed methylation-specific probes.

Any suitable methylation-discriminatory microarrays may be employed to assess the methylation status of the CpGs described herein. One particular methylation-discriminatory microarray system is provided by Illumina, Inc. (San Diego, CA; http://www.illumina.com/). In particular, the Infinium MethylationEPIC BeadChip array and the Infinium HumanMethylation450 BeadChip array systems may be used to assess the methylation status of CpGs for predicting cancer development as described herein. Such a system exploits the chemical modifications made to DNA following bisulphite treatment of the starting DNA molecule. Briefly, the array comprises beads to which are coupled oligonucleotide probes specific for DNA sequences corresponding to the unmethylated form of a CpG, as well as separate beads to which are coupled oligonucleotide probes specific for DNA sequences corresponding to the methylated form of an CpG. Candidate DNA molecules are applied to the array and selectively hybridize, under appropriate conditions, to the oligonucleotide probe corresponding to the relevant epigenetic form. Thus, a DNA molecule derived from a CpG which was methylated in the corresponding genomic DNA will selectively attach to the bead comprising the methylation-specific oligonucleotide probe, but will fail to attach to the bead comprising the non-methylation-specific oligonucleotide probe. Single-base extension of only the hybridized probes incorporates a labeled ddNTP, which is subsequently stained with a fluorescence reagent and imaged. The methylation status of the CpG is determined by calculating the ratio of the fluorescent signal derived from the methylated and unmethylated sites.

Infinium HumanMethylation450 BeadChip array systems can be used to interrogate CpGs in the assays described herein. Alternative or customised arrays could, however, be employed to interrogate the cancer-specific CpG biomarkers defined herein, provided that they comprise means for interrogating all CpG for a given assay, as defined herein.

Techniques involving combinations of the above-described assays may also be used. For example, DNA containing CpG sequences of interest may be hybridized to microarrays and then subjected to DNA sequencing to determine the status of the CpG as described above.

In the assays described above, sequences corresponding to CpG loci may also be subjected to an enrichment process if desired. DNA containing CpG sequences of interest may be captured by binding molecules such as oligonucleotide probes complementary to the CpG target sequence of interest. Sequences corresponding to CpG loci may be captured before or after bisulphite conversion or before or after amplification. Probes may be designed to be complementary to bisulphite converted DNA. Captured DNA may then be subjected to further processing steps to determine the status of the CpG, such as DNA sequencing steps.

Capture/separation steps may be custom designed. Alternatively a variety of such techniques are available commercially, e.g. the SureSelect target enrichment system available from Agilent Technologies (http://www.agilent.com/home). In this system biotinylated “bait” or “probe” sequences (e.g. RNA) complementary to the DNA containing CpG sequences of interest are hybridized to sample nucleic acids. Streptavidin-coated magnetic beads are then used to capture sequences of interest hybridized to bait sequences. Unbound fractions are discarded. Bait sequences are then removed (e.g. by digestion of RNA) thus providing an enriched pool of CpG target sequences separated from non-CpG sequences. Template DNA may be subjected to bisulphite conversion and target loci amplified by small-scale PCR such as microdroplet PCR using primers which are independent of the methylation status of the CpG. Following amplification, samples may be subjected to a capture step to enrich for PCR products containing the target CpG, e.g. captured and purified using magnetic beads, as described above. Following capture, a standard PCR reaction is carried out to incorporate DNA sequencing barcodes into CpG-containing amplicons. PCR products are again purified and then subjected to DNA sequencing and analysis to determine the presence or absence of a methylcytosine at the target genomic CpG.

CpG biomarker loci defined herein by SEQ ID NOs 1 to 5000 correspond to Illumina® identifiers (IlmnID) known in the art. These CpG loci identifiers refer to individual CpG sites used in the commercially available Illumina® Infinium Methylation EPIC BeadChip kit and Illumina® Infinium Human Methylation450 BeadChip kit. The identity of each CpG site represented by each CpG loci identifier is publicly available from the Illumina, Inc. website under reference to the CpG sites used in the Infinium Methylation EPIC BeadChip kit and the Infinium Human Methylation450 BeadChip kit.

To complement evolving public databases to provide accurate CpG loci identifiers and strand orientation, Illumina® has developed a method to consistently designate CpG loci based on the actual or contextual sequence of each individual CpG locus. To unambiguously refer to CpG loci in any species, Illumina® has developed a consistent and deterministic CpG loci database to ensure uniformity in the reporting of methylation data. The Illumina® method takes advantage of sequences flanking a CpG locus to generate a unique CpG locus cluster ID. This number is based on sequence information only and is unaffected by genome version. Illumina's standardized nomenclature also parallels the TOP/BOT strand nomenclature (which indicates the strand orientation) commonly used for single nucleotide polymorphism (SNP) designation.

Illumina® Identifiers for the Infinium MethylationEPIC BeadChip and Infinium Human Methylation450 BeadChip system are also available from public repositories such as Gene Expression Omnibus (GEO) (http://www.ncbi.nlm.nih.gov/geo/).

By assessing the methylation status of a CpG it is meant that a determination is made as to whether a given CpG is methylated or unmethylated. In addition, it is meant that a determination is made as to the degree to which a given CpG site is methylated across a population of CpG loci in a sample.

CpG methylation status may be measured indirectly using a detection system such as fluorescence. A methylation-discriminatory microarray may be used. When calculating the degree of methylation of a given CpG, the Illumina® definition of beta-values may be used. The Illumina® methylation beta-value of a specific CpG site is calculated from the intensity of the methylated (M) and unmethylated (U) alleles, as the ratio of fluorescent signals β=Max(M,0)/[Max(M,0)+Max(U,0)+100]. On this scale, 0<β<1, β values of 1 or close to 1 indicate 100% methylation whereas values of 0 or close to 0 indicate 0% methylation.

The methylation status of any one or more CpGs of the CpGs defined by SEQ ID NOs: 1 to 5000 or identified in SEQ ID NOs: 5001 to 5418 may be assessed by any suitable technique. As explained in more detail in the Examples below, one particular exemplary technique which the inventors have used is a methylation discriminatory array, such as an Illumina InfiniumMethylation EPIC BeadChip. These assays utilise probes directed to methylated and unmethylated CpGs at a given locus.

Another exemplary technique which the inventors have used to determine the methylation status of any one or more CpGs is a fluorescence-based PCR technique referred to as MethyLight. These assays utilise forward and reverse PCR primers specific for sequences encompassing any one or more of the 5000 CpGs defined according to SEQ ID NOS: 1 to 5000, or identified in SEQ ID NOs: 5001 to 5418 and 5703 to 5786. The methylation status of one or more of the CpGs defined by SEQ ID NOs: 1 to 5000, or identified in SEQ ID NOs: 5001 to 5418 and 5703 to 5786 may therefore be determined by MethyLight analysis. The detectable probes are typically designed such that they hybridise only to methylated forms of the one or more CpGs to be assayed in view of the bisulfite conversion step within a typical MethyLight protocol.

### Bioinformatic Tools and Statistical Metrics for CpG-Based Assays

Software programs which aid in the in silico analysis of bisulphite converted DNA sequences and in primer design for the purposes of methylation-specific analyses are generally available and have been described previously.

In risk models for predicting cancer, a receiver-operating-characteristic (ROC) curve analysis often used, in which the area under the curve (AUC) is assessed. Each point on the ROC curve shows the effect of a rule for turning a risk/likelihood estimate into a prediction of the presence, absence or development of CIN3 and/or cancer, particularly cervical or endometrial cancer, most preferably cervical cancer, in an individual. The AUC measures how well the model discriminates between case subjects and control subjects. An ROC curve that corresponds to a random classification of case subjects and control subjects is a straight line with an AUC of 50%. An ROC curve that corresponds to perfect classification has an AUC of 100%.

In any of the methods described herein, the 95% confidence interval for the ROC AUC may be between 0.60 and 1.

In any of the methods described herein, the interval may be defined as a range having as an upper limit any number between 0.60 and 1. The upper limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval may be defined as a range having as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

In any of the methods described herein, the interval range may comprise any of the above lower limit numbers combined with any of the above upper limit numbers as appropriate.

Preferably, the upper limit number is 1. Thus, the 95% confidence ROC AUC interval may be defined as a range having an upper limit of 1 and as a lower limit any number between 0.60 and 1. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99 or 1.00.

The upper limit number may be 0.99. Thus, the 95% confidence ROC AUC interval may be defined as a range having an upper limit of 0.99 and as a lower limit any number between 0.60 and 0.99. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or 0.99.

The upper limit number may be 0.98. Thus, the 95% confidence ROC AUC interval may be defined as a range having an upper limit of 0.98 and as a lower limit any number between 0.60 and 0.98. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97 or 0.98.

The upper limit number may be 0.97. Thus, the 95% confidence ROC AUC interval may be defined as a range having an upper limit of 0.97 and as a lower limit any number between 0.60 and 0.97. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96 or 0.97.

The upper limit number may be 0.96. Thus, the 95% confidence ROC AUC interval may be defined as a range having an upper limit of 0.96 and as a lower limit any number between 0.60 and 0.96. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95 or 0.96.

The upper limit number may be 0.95. Thus, the 95% confidence ROC AUC interval may be defined as a range having an upper limit of 0.95 and as a lower limit any number between 0.60 and 0.95. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94 or 0.95.

The upper limit number may be 0.94. Thus, the 95% confidence ROC AUC interval may be defined as a range having an upper limit of 0.94 and as a lower limit any number between 0.60 and 0.94. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93 or 0.94.

The upper limit number may be 0.93. Thus, the 95% confidence ROC AUC interval may be defined as a range having an upper limit of 0.93 and as a lower limit any number between 0.60 and 0.93. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92 or 0.93.

The upper limit number may be 0.92. Thus, the 95% confidence ROC AUC interval may be defined as a range having an upper limit of 0.92 and as a lower limit any number between 0.60 and 0.92. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91 or 0.92.

The upper limit number may be 0.91. Thus, the 95% confidence ROC AUC interval may be defined as a range having an upper limit of 0.91 and as a lower limit any number between 0.60 and 0.91. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90 or 0.91.

The upper limit number may be 0.90. Thus, the 95% confidence ROC AUC interval may be defined as a range having an upper limit of 0.90 and as a lower limit any number between 0.60 and 0.90. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89 or 0.90.

The upper limit number may be 0.89. Thus, the 95% confidence ROC AUC interval may be defined as a range having an upper limit of 0.89 and as a lower limit any number between 0.60 and 0.89. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88 or 0.89.

The upper limit number may be 0.88. Thus, the 95% confidence ROC AUC interval may be defined as a range having an upper limit of 0.88 and as a lower limit any number between 0.60 and 0.88. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87 or 0.88.

The upper limit number may be 0.87. Thus, the 95% confidence ROC AUC interval may be defined as a range having an upper limit of 0.87 and as a lower limit any number between 0.60 and 0.87. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86 or 0.87.

The upper limit number may be 0.86. Thus, the 95% confidence ROC AUC interval may be defined as a range having an upper limit of 0.86 and as a lower limit any number between 0.60 and 0.86. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85 or 0.86.

The upper limit number may be 0.85. Thus, the 95% confidence ROC AUC interval may be defined as a range having an upper limit of 0.85 and as a lower limit any number between 0.60 and 0.85. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84 or 0.85.

The upper limit number may be 0.84. Thus, the 95% confidence ROC AUC interval may be defined as a range having an upper limit of 0.84 and as a lower limit any number between 0.60 and 0.84. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83 or 0.84.

The upper limit number may be 0.83. Thus, the 95% confidence ROC AUC interval may be defined as a range having an upper limit of 0.83 and as a lower limit any number between 0.60 and 0.83. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82 or 0.83.

The upper limit number may be 0.82. Thus, the 95% confidence ROC AUC interval may be defined as a range having an upper limit of 0.82 and as a lower limit any number between 0.60 and 0.82. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81 or 0.82.

The upper limit number may be 0.81. Thus, the 95% confidence ROC AUC interval may be defined as a range having an upper limit of 0.81 and as a lower limit any number between 0.60 and 0.81. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80 or 0.81.

The upper limit number may be 0.80. Thus, the 95% confidence ROC AUC interval may be defined as a range having an upper limit of 0.80 and as a lower limit any number between 0.60 and 0.80. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79 or 0.80.

The upper limit number may be 0.79. Thus, the 95% confidence ROC AUC interval may be defined as a range having an upper limit of 0.79 and as a lower limit any number between 0.60 and 0.79. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78 or 0.79.

The upper limit number may be 0.78. Thus, the 95% confidence ROC AUC interval may be defined as a range having an upper limit of 0.78 and as a lower limit any number between 0.60 and 0.78. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76, 0.77 or 0.78.

The upper limit number may be 0.77. Thus, the 95% confidence ROC AUC interval may be defined as a range having an upper limit of 0.77 and as a lower limit any number between 0.60 and 0.77. The lower limit number may be 0.60, 0.61, 0.62, 0.63, 0.64, 0.65, 0.66, 0.67, 0.68, 0.69, 0.70, 0.71, 0.72, 0.73, 0.74, 0.75, 0.76 or 0.77.

The upper limit number may be 0.76. Thus, the 95% confidence ROC AUC interval may be defined as a range having an upper limit of 0.76 and as a lower limit any number between 0.60 and 0.76. The lower limit number may be 0.60, 0.61, 0.62, 0.63, 0.64, 0.65, 0.66, 0.67, 0.68, 0.69, 0.70, 0.71, 0.72, 0.73, 0.74, 0.75 or 0.76.

The upper limit number may be 0.75. Thus, the 95% confidence ROC AUC interval may be defined as a range having an upper limit of 0.75 and as a lower limit any number between 0.60 and 0.75. The lower limit number may be 0.60, 0.61, 0.62, 0.63, 0.64, 0.65, 0.66, 0.67, 0.68, 0.69, 0.70, 0.71, 0.72, 0.73, 0.74 or 0.75.

The upper limit number may be 0.74. Thus, the 95% confidence ROC AUC interval may be defined as a range having an upper limit of 0.74 and as a lower limit any number between 0.60 and 0.74. The lower limit number may be 0.60, 0.61, 0.62, 0.63, 0.64, 0.65, 0.66, 0.67, 0.68, 0.69, 0.70, 0.71, 0.72, 0.73 or 0.74.

The upper limit number may be 0.73. Thus, the 95% confidence ROC AUC interval may be defined as a range having an upper limit of 0.73 and as a lower limit any number between 0.60 and 0.73. The lower limit number may be 0.60, 0.61, 0.62, 0.63, 0.64, 0.65, 0.66, 0.67, 0.68, 0.69, 0.70, 0.71, 0.72 or 0.73.

The upper limit number may be 0.72. Thus, the 95% confidence ROC AUC interval may be defined as a range having an upper limit of 0.72 and as a lower limit any number between 0.60 and 0.72. The lower limit number may be 0.60, 0.61, 0.62, 0.63, 0.64, 0.65, 0.66, 0.67, 0.68, 0.69, 0.70, 0.71 or 0.72.

The upper limit number may be 0.71. Thus, the 95% confidence ROC AUC interval may be defined as a range having an upper limit of 0.71 and as a lower limit any number between 0.60 and 0.71. The lower limit number may be 0.60, 0.61, 0.62, 0.63, 0.64, 0.65, 0.66, 0.67, 0.68, 0.69, 0.70 or 0.71.

The upper limit number may be 0.70. Thus, the 95% confidence ROC AUC interval may be defined as a range having an upper limit of 0.70 and as a lower limit any number between 0.60 and 0.70. The lower limit number may be 0.60, 0.61, 0.62, 0.63, 0.64, 0.65, 0.66, 0.67, 0.68, 0.69 or 0.70.

### Methods of Treatment and Diagnosis

The term “treatment” as used herein is intended to refer to any intervention or procedure performed on an individual, including a surgical intervention or a pharmacological intervention such as the administration of a compound or drug. Any such treatment may be performed for therapeutic purposes or for preventative or prophylactic purposes.

The invention also encompasses the performance of one or more treatment steps following a positive classification of CIN3 and/or cancer, particularly cervical and/or endometrial cancer, based on any of the methods described herein. Said treatments may be considered “therapeutic” treatments.

The invention also encompasses the performance of one or more treatment steps following a negative classification of CIN3 and/or cancer or prediction of an individual being at risk of cancer development, particularly cervical and/or endometrial cancer, based on any of the methods described herein. Said treatments may be considered “risk prevention”, “preventative” or “prophylactic” treatments.

The invention also encompasses the performance of one or more treatment steps following a negative classification of CIN3 and/or cancer or prediction of an individual being at risk of CIN3 and/or cancer development based on any of the methods described herein, in an individual that harbours one or more mutations that predispose the individual to an increased risk of developing CIN3 and/or cancer.

The invention thus encompasses a method of treating a cancer and/or CIN3 patient comprising administering chemotherapy, radiation, immunotherapy or any therapy described herein to the patient determined to have a cancer index value which indicates that the patient has is positive for cancer and/or CIN3 based on any of the assays described herein, preferably wherein the cancer is cervical cancer.

The invention thus encompasses a method of treating and/or preventing CIN3 and/or cancer in an individual, the method comprising:
a. assessing the cancer status of the individual by assessing the presence, absence or development of CIN3 and/or cancer, particularly cervical or endometrial cancer, most preferably cervical cancer, in the individual by performing any one of the assays described herein;
b. administering one or more therapeutic or preventative treatments to the individual based on the assessment, preferably wherein the cancer is cervical and/or endometrial cancer, most preferably cervical cancer.

The invention thus encompasses a method of treating and/or preventing cancer in an individual, the method comprising:
a. assessing the cancer status of the individual by assessing the presence, absence or development of CIN3 and/or cancer, particularly cervical or endometrial cancer, most preferably cervical cancer, in the individual comprising:
   i. providing a sample which has been taken from the individual, the sample comprising a population of DNA molecules;
   ii. determining in the population of DNA molecules in the sample the methylation status of a panel of:
      1. one or more CpGs selected from a panel of CpGs identified in SEQ ID NOs 1 to 5000 wherein the CpGs are identified at nucleotide positions 61 to 62; and/or
      2. one or more CpGs selected from within a panel of one or more Differentially Methylated Regions (DMRs) defined by SEQ ID NOs 5001 to 5418, wherein the CpGs are denoted by CG;
   iii. deriving a cancer index value based on the methylation status of the one or more CpGs in the panel; and
   iv. assessing the presence, absence or development of CIN3 and/or cancer, particularly cervical or endometrial cancer, most preferably cervical cancer, in the individual based on the cancer index value, wherein the assay is characterised as having an area under the curve (AUC) of 0.60 or more as determined by receiver operating characteristics (ROC);
b. administering one or more therapeutic treatments to the individual based on the assessment,
preferably wherein the cancer in cervical and/or endometrial cancer, most preferably cervical cancer.

In any of the methods of treatment encompassed by the invention, the step of predicting the presence or development of CIN3 and/or cancer, preferably wherein the cancer in cervical and/or endometrial cancer, in an individual may involve deriving a cancer index value.

In any of the methods of treatment encompassed by the invention, the step of predicting the presence or development of CIN3 and/or cancer in an individual may involve the use of any one of the arrays described herein.

In any of the methods of treatment encompassed by the invention, the step of stratifying the individual may involve applying any one of the thresholds according to any one of the assays of the invention described herein.

The step of administering one or more treatments may comprise different treatment steps depending on the stratification of an individual on the basis of their CIN3 and/or cancer status or their risk of having CIN3 and/or cancer or on the basis of risk of CIN3 and/or cancer development, particularly cervical and/or endometrial cancer, most preferably cervical cancer. Particularly the amount of an invasiveness of the treatments administered may vary dependent on the stratification of an individual on the basis of their CIN3 and/or cancer status or their risk of having CIN3 and/or cancer or on the basis of their risk of CIN3 and/or cancer development. The treatments administered to the individual may comprise any treatments considered suitable by a person skilled in the art.

For example, when the individual is assessed as not having CIN3 and/or cancer, as having a low risk of having CIN3 and/or cancer or as having a low risk of CIN3 and/or cancer development, and wherein the cancer index value is about −0.530 or more and less than −0.330, and preferably wherein the assay comprises determining methylation β-values for each CpG in the panel of one or more CpGs the individual is subjected to one or more treatments according to cancer index value, the one or more treatments may comprise a repeat assay according to the assays of the invention described herein, preferably wherein the repeat assay is performed about one year after the previous assay, preferably wherein the cancer is cervical and/or endometrial cancer, most preferably wherein the cancer is cervical cancer.

For example, when the individual is assessed as having a moderate risk of having CIN3 and/or cancer or as having a moderate risk of CIN3 and/or cancer development, and wherein the cancer index value is about −0.330 or more and less than −0.170, and preferably wherein the assay comprises determining methylation β-values for each CpG in the panel of one or more CpGs the individual is subjected to one or more treatments according to cancer index value, the one or more treatments may comprise a test for human papilloma virus (HPV) status and wherein:
1. when the individual is HPV positive, a colposcopy, and optionally a transvaginal ultrasound and/or an endometrial biopsy to assess endometrium; or
2. when the individual is HPV negative, a repeat assay according to any one of the assays of the invention described herein, preferably wherein the repeat assay is performed about one year after the previous assay,
preferably wherein the cancer is cervical and/or endometrial cancer, most
preferably wherein the cancer is cervical cancer.

For example, when the individual is assessed as having CIN3 and/or cancer or as having a high risk of CIN3 and/or cancer development, and wherein the cancer index value is about −0.170 or more, and preferably wherein the assay comprises determining methylation β-values for each CpG in the panel of one or more CpGs the individual is subjected to one or more treatments according to cancer index value, the one or more treatments may comprise a colposcopy, and wherein the colposcopy is negative, an endometrial biopsy and hysteroscopy.

For example, when the individual is assessed as having CIN3 and/or cancer or as having a high risk of CIN3 and/or cancer development, and wherein the cancer index value is about:
a. 5.000 or more, preferably wherein the step of determining the methylation status of the one or more CpGs in the panel comprises determining each CpG within SEQ ID NO 5768 and/or SEQ ID NO 5712 and/or SEQ ID NO 5740;
b. 1.000 or more, preferably wherein the step of determining the methylation status of the one or more CpGs in the panel comprises determining each CpG within SEQ ID NO 5765 and/or SEQ ID NO 5709 and/or SEQ ID NO 5737;
c. 2.000 or more, preferably wherein the step of determining the methylation status of the one or more CpGs in the panel comprises determining each CpG within SEQ ID NO 5759 and/or SEQ ID NO 5703 and/or SEQ ID NO 5731;
d. 5.000 or more, preferably wherein the step of determining the methylation status of the one or more CpGs in the panel comprises determining each CpG within SEQ ID NO 5785 and/or SEQ ID NO 5729 and/or SEQ ID NO 5757;
e. 4.000 or more, preferably wherein the step of determining the methylation status of the one or more CpGs in the panel comprises determining each CpG within SEQ ID NO 5763 and/or SEQ ID NO 5707 and/or SEQ ID NO 5735;
f. 4.000 or more, preferably wherein the step of determining the methylation status of the one or more CpGs in the panel comprises determining each CpG within SEQ ID NO 5781 and/or SEQ ID NO 5725 and/or SEQ ID NO 5753;
g. 1.000 or more, preferably wherein the step of determining the methylation status of the one or more CpGs in the panel comprises determining each CpG within SEQ ID NO 5769 and/or SEQ ID NO 5713 and/or SEQ ID NO 5741;
h. 1.000 or more, preferably wherein the step of determining the methylation status of the one or more CpGs in the panel comprises determining each CpG within SEQ ID NO 5766 and/or SEQ ID NO 5710 and/or SEQ ID NO 5738;
i. 4.000 or more, preferably wherein the step of determining the methylation status of the one or more CpGs in the panel comprises determining each CpG within SEQ ID NO 5761 and/or SEQ ID NO 5705 and/or SEQ ID NO 5733;
j. 4.000 or more, preferably wherein the step of determining the methylation status of the one or more CpGs in the panel comprises determining each CpG within SEQ ID NO 5779 and/or SEQ ID NO 5723 and/or SEQ ID NO 5751;
k. 4.000 or more, preferably wherein the step of determining the methylation status of the one or more CpGs in the panel comprises determining each CpG within SEQ ID NO 5764 and/or SEQ ID NO 5708 and/or SEQ ID NO 5736;
l. 5.000 or more, preferably wherein the step of determining the methylation status of the one or more CpGs in the panel comprises determining each CpG within SEQ ID NO 5772 and/or SEQ ID NO 5716 and/or SEQ ID NO 5744;
m. 2.000 or more, preferably wherein the step of determining the methylation status of the one or more CpGs in the panel comprises determining each CpG within SEQ ID NO 5773 and/or SEQ ID NO 5717 and/or SEQ ID NO 5745;
n. 1.000 or more, preferably wherein the step of determining the methylation status of the one or more CpGs in the panel comprises determining each CpG within SEQ ID NO 5778 and/or SEQ ID NO 5722 and/or SEQ ID NO 5750;
o. 5.000 or more, preferably wherein the step of determining the methylation status of the one or more CpGs in the panel comprises determining each CpG within SEQ ID NO 5767 and/or SEQ ID NO 5711 and/or SEQ ID NO 5739;
p. 5.000 or more, preferably wherein the step of determining the methylation status of the one or more CpGs in the panel comprises determining each CpG within SEQ ID NO 5774 and/or SEQ ID NO 5718 and/or SEQ ID NO 5746;
q. more than 0.000, preferably wherein the step of determining the methylation status of the one or more CpGs in the panel comprises determining each CpG within SEQ ID NO 5760 and/or SEQ ID NO 5704 and/or SEQ ID NO 5732;
r. 4.000 or more, preferably wherein the step of determining the methylation status of the one or more CpGs in the panel comprises determining each CpG within SEQ ID NO 5771 and/or SEQ ID NO 5715 and/or SEQ ID NO 5743;
s. 2.000 or more, preferably wherein the step of determining the methylation status of the one or more CpGs in the panel comprises determining each CpG within SEQ ID NO 5762 and/or SEQ ID NO 5706 and/or SEQ ID NO 5734;
t. 7.000 or more, preferably wherein the step of determining the methylation status of the one or more CpGs in the panel comprises determining each CpG within SEQ ID NO 5780 and/or SEQ ID NO 5724 and/or SEQ ID NO 5752;
u. 6.000 or more, preferably wherein the step of determining the methylation status of the one or more CpGs in the panel comprises determining each CpG within SEQ ID NO 5776 and/or SEQ ID NO 5720 and/or SEQ ID NO 5748;
v. 5.000 or more, preferably wherein the step of determining the methylation status of the one or more CpGs in the panel comprises determining each CpG within SEQ ID NO 5783 and/or SEQ ID NO 5727 and/or SEQ ID NO 5755;
w. 6.000 or more, preferably wherein the step of determining the methylation status of the one or more CpGs in the panel comprises determining each CpG within SEQ ID NO 5782 and/or SEQ ID NO 5726 and/or SEQ ID NO 5754;
x. 6.000 or more, preferably wherein the step of determining the methylation status of the one or more CpGs in the panel comprises determining each CpG within SEQ ID NO 5784 and/or SEQ ID NO 5728 and/or SEQ ID NO 5756;
y. 5.000 or more, preferably wherein the step of determining the methylation status of the one or more CpGs in the panel comprises determining each CpG within SEQ ID NO 5777 and/or SEQ ID NO 5721 and/or SEQ ID NO 5749;
z. 2.000 or more, preferably wherein the step of determining the methylation status of the one or more CpGs in the panel comprises determining each CpG within SEQ ID NO 5786 and/or SEQ ID NO 5730 and/or SEQ ID NO 5758;
aa. 7.000 or more, preferably wherein the step of determining the methylation status of the one or more CpGs in the panel comprises determining each CpG within SEQ ID NO 5775 and/or SEQ ID NO 5719 and/or SEQ ID NO 5747; or
bb. 3.000 or more, preferably wherein the step of determining the methylation status of the one or more CpGs in the panel comprises determining each CpG within SEQ ID NO 5770 and/or SEQ ID NO 5714 and/or SEQ ID NO 5742,
and more preferably wherein the assay comprises determining percent methylated reference for the sequence defined by the specified SEQ ID NO, the individual is subjected to one or more treatments according to their cancer index value, the one or more treatments comprise a colposcopy, and wherein the colposcopy is negative, an endometrial biopsy, and wherein the endometrial biopsy is also negative, a repeat assay according to any one of assays of the invention described herein, preferably wherein the repeat assay is performed about one year after the previous assay, or when the individual's cancer index value according to 1. to 28 is less than the specified cancer index value no treatment is administered to the individual.

In any of the assays described herein, the one or more treatments that the individual is subjected may be repeated at any suitable interval as would be understood by a person skilled in the art. For example, any one of the one or more treatments that the individual is subjected to are repeated on a monthly, three monthly, six monthly, yearly or two yearly basis following an initial administration.

Other exemplary treatments comprise one or more surgical procedures, one or more chemotherapeutic agents, one or more cytotoxic chemotherapeutic agents one or more radiotherapeutic agents, one or more immunotherapeutic agents, one or more biological therapeutics, one or more anti-hormonal treatments or any combination of the above following a positive diagnosis of cancer.

In any of the methods of treatment described herein, the individual may particularly be administered treatments recited in Table 9. Four sub-groups defined by ranges of cancer index values are specified in Table 9 as corresponding to preferred clinical actions, comprising intensified screening, administration of therapeutics and surgery.

Cancer treatments may be administered to an individual harbouring cancer or at risk of cancer development, in an amount sufficient to prevent, treat, cure, alleviate or partially arrest cancer or one or more of its symptoms. Such treatments may result in a decrease in severity, and/or decreased cancer index value, of cancer symptoms, or an increase in frequency or duration of symptom-free periods. A treatment amount adequate to accomplish this defined as “therapeutically effective amount”. Effective amounts for a given purpose will depend on the severity of cancer and/or the individual's cancer index value as well as the weight and general state of the individual. As used herein, the term “individual” includes any human, preferably wherein the human is a woman. As used herein, “treatment” is to be considered synonymous with “therapeutic agent”.

The following therapeutic agents may be administered to an individual based on their cancer risk alone or in combination with any other treatment described herein. The therapeutic agent may be directly attached, for example by chemical conjugation, to an antibody. Methods of conjugating agents or labels to an antibody are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-β-dimethylaminopropyl) carbodiimide (EDC) is particularly useful for conjugating a functional moiety to a binding moiety. Other methods for conjugating a moiety to antibodies can also be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the antibody maintains its targeting ability and that the functional moiety maintains its relevant function.

A cytotoxic moiety may be directly and/or indirectly cytotoxic. By “directly cytotoxic” it is meant that the moiety is one which on its own is cytotoxic. By “indirectly cytotoxic” it is meant that the moiety is one which, although is not itself cytotoxic, can induce cytotoxicity, for example by its action on a further molecule or by further action on it. The cytotoxic moiety may be cytotoxic only when intracellular and is preferably not cytotoxic when extracellular.

Cytotoxic chemotherapeutic agents are well known in the art. Cytotoxic chemotherapeutic agents, such as anticancer agents, include: alkylating agents including nitrogen mustards such as mechlorethamine (HN2), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

A cytotoxic chemotherapeutic agent may be a cytotoxic peptide or polypeptide moiety which leads to cell death. Cytotoxic peptide and polypeptide moieties are well known in the art and include, for example, ricin, abrin, Pseudomonas exotoxin, tissue factor and the like. Methods for linking them to targeting moieties such as antibodies are also known in the art. Other ribosome inactivating proteins are described as cytotoxic agents in WO 96/06641. Pseudomonas exotoxin may also be used as the cytotoxic polypeptide. Certain cytokines, such as TNFα and IL-2, may also be useful as cytotoxic agents.

Certain radioactive atoms may also be cytotoxic if delivered in sufficient doses. Radiotherapeutic agents may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to an antibody, antigen-binding fragment, variant, fusion or derivative thereof in known ways. For example, EDTA or another chelating agent may be attached to the binding moiety and used to attach 111In or 90Y. Tyrosine residues may be directly labelled with 1251 or 1311.

A cytotoxic chemotherapeutic agent may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. With antibodies, this type of system is often referred to as ADEPT (Antibody-Directed Enzyme Prodrug Therapy). The system requires that the antibody locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues. In a preferred embodiment, the cytotoxic moiety is capable of converting a non-cytotoxic prodrug into a cytotoxic drug.

In any of the methods of treatment described herein, the one or more treatments that the individual is subjected to may be repeated on one or more occasions. The one or more treatments may be repeated at regular intervals. The repetitive nature of the treatment administration may depend on the particular treatment being administered. Some treatments may require repetitive administration at greater frequency than others. The skilled person would be aware of the frequency of administration required for therapies known in the art. The one or more treatments may be repeated weekly, two weekly, three weekly, four weekly, monthly, three monthly, six monthly, yearly, two yearly, three yearly, four yearly, or five yearly.

In any of the methods described herein, when the individual is assessed as having a cancer index value of is less than about −0.530, and preferably wherein the assay comprises determining methylation β-values for each CpG in the panel of one or more CpGs, no treatment is administered to the individual.

In any of the methods described herein, when the individual is assessed as having a cancer index value of is:
1. less than about 5.000, and preferably wherein the step of determining the methylation status of the one or more CpGs in the panel comprises determining each CpG within SEQ ID NO 5768 and/or SEQ ID NO 5712 and/or SEQ ID NO 5740, and more preferably wherein the assay comprises determining percent methylated reference for the sequence defined by the specified SEQ ID NO, no treatment is administered to the individual;
2. less than about 1.000, and preferably wherein the step of determining the methylation status of the one or more CpGs in the panel comprises determining each CpG within SEQ ID NO 5765 and/or SEQ ID NO 5709 and/or SEQ ID NO 5737, and more preferably wherein the assay comprises determining percent methylated reference for the sequence defined by the specified SEQ ID NO, no treatment is administered to the individual;
3. less than about 2.000, and preferably wherein the step of determining the methylation status of the one or more CpGs in the panel comprises determining each CpG within SEQ ID NO 5759 and/or SEQ ID NO 5703 and/or SEQ ID NO 5731, and more preferably wherein the assay comprises determining percent methylated reference for the sequence defined by the specified SEQ ID NO, no treatment is administered to the individual;
4. less than about 5.000, and preferably wherein the step of determining the methylation status of the one or more CpGs in the panel comprises determining each CpG within SEQ ID NO 5785 and/or SEQ ID NO 5729 and/or SEQ ID NO 5757, and more preferably wherein the assay comprises determining percent methylated reference for the sequence defined by the specified SEQ ID NO, no treatment is administered to the individual;
5. less than about 4.000, and preferably wherein the step of determining the methylation status of the one or more CpGs in the panel comprises determining each CpG within SEQ ID NO 5763 and/or SEQ ID NO 5707 and/or SEQ ID NO 5735, and more preferably wherein the assay comprises determining percent methylated reference for the sequence defined by the specified SEQ ID NO, no treatment is administered to the individual;
6. less than about 4.000, and preferably wherein the step of determining the methylation status of the one or more CpGs in the panel comprises determining each CpG within SEQ ID NO 5781 and/or SEQ ID NO 5725 and/or SEQ ID NO 5753, and more preferably wherein the assay comprises determining percent methylated reference for the sequence defined by the specified SEQ ID NO, no treatment is administered to the individual;
7. less than about 1.000, and preferably wherein the step of determining the methylation status of the one or more CpGs in the panel comprises determining each CpG within SEQ ID NO 5769 and/or SEQ ID NO 5713 and/or SEQ ID NO 5741, and more preferably wherein the assay comprises determining percent methylated reference for the sequence defined by the specified SEQ ID NO, no treatment is administered to the individual;
8. less than about 1.000, and preferably wherein the step of determining the methylation status of the one or more CpGs in the panel comprises determining each CpG within SEQ ID NO 5766 and/or SEQ ID NO 5710 and/or SEQ ID NO 5738, and more preferably wherein the assay comprises determining percent methylated reference for the sequence defined by the specified SEQ ID NO, no treatment is administered to the individual;
9. less than about 4.000, and preferably wherein the step of determining the methylation status of the one or more CpGs in the panel comprises determining each CpG within SEQ ID NO 5761 and/or SEQ ID NO 5705 and/or SEQ ID NO 5733, and more preferably wherein the assay comprises determining percent methylated reference for the sequence defined by the specified SEQ ID NO, no treatment is administered to the individual;
10. less than about 4.000, and preferably wherein the step of determining the methylation status of the one or more CpGs in the panel comprises determining each CpG within SEQ ID NO 5779 and/or SEQ ID NO 5723 and/or SEQ ID NO 5751, and more preferably wherein the assay comprises determining percent methylated reference for the sequence defined by the specified SEQ ID NO, no treatment is administered to the individual;
11. less than about 4.000, and preferably wherein the step of determining the methylation status of the one or more CpGs in the panel comprises determining each CpG within SEQ ID NO 5764 and/or SEQ ID NO 5708 and/or SEQ ID NO 5736, and more preferably wherein the assay comprises determining percent methylated reference for the sequence defined by the specified SEQ ID NO, no treatment is administered to the individual;
12. less than about 5.000, and preferably wherein the step of determining the methylation status of the one or more CpGs in the panel comprises determining each CpG within SEQ ID NO 5772 and/or SEQ ID NO 5716 and/or SEQ ID NO 5744, and more preferably wherein the assay comprises determining percent methylated reference for the sequence defined by the specified SEQ ID NO, no treatment is administered to the individual;
13. less than about 2.000, and preferably wherein the step of determining the methylation status of the one or more CpGs in the panel comprises determining each CpG within SEQ ID NO 5773 and/or SEQ ID NO 5717 and/or SEQ ID NO 5745, and more preferably wherein the assay comprises determining percent methylated reference for the sequence defined by the specified SEQ ID NO, no treatment is administered to the individual;
14. less than about 1.000, and preferably wherein the step of determining the methylation status of the one or more CpGs in the panel comprises determining each CpG within SEQ ID NO 5778 and/or SEQ ID NO 5772 and/or SEQ D NO 5750, and more preferably wherein the assay comprises determining percent methylated reference for the sequence defined by the specified SEQ ID NO, no treatment is administered to the individual;
15. less than about 5.000, and preferably wherein the step of determining the methylation status of the one or more CpGs in the panel comprises determining each CpG within SEQ ID NO 5767 and/or SEQ ID NO 5711 and/or SEQ ID NO 5739, and more preferably wherein the assay comprises determining percent methylated reference for the sequence defined by the specified SEQ ID NO, no treatment is administered to the individual;
16. less than about 5.000, and preferably wherein the step of determining the methylation status of the one or more CpGs in the panel comprises determining each CpG within SEQ ID NO 5774 and/or SEQ ID NO 5718 and/or SEQ ID NO 5746, and more preferably wherein the assay comprises determining percent methylated reference for the sequence defined by the specified SEQ ID NO, no treatment is administered to the individual;
17. 0.000, preferably wherein the step of determining the methylation status of the one or more CpGs in the panel comprises determining each CpG within SEQ ID NO 5760 and/or SEQ ID NO 5704 and/or SEQ ID NO 5732, and more preferably wherein the assay comprises determining percent methylated reference for the sequence defined by the specified SEQ ID NO, no treatment is administered to the individual;
18. less than about 4.000, and preferably wherein the step of determining the methylation status of the one or more CpGs in the panel comprises determining each CpG within SEQ ID NO 5771 and/or SEQ ID NO 5715 and/or SEQ ID NO 5743, and more preferably wherein the assay comprises determining percent methylated reference for the sequence defined by the specified SEQ ID NO, no treatment is administered to the individual;
19. less than about 2.000, and preferably wherein the step of determining the methylation status of the one or more CpGs in the panel comprises determining each CpG within SEQ ID NO 5762 and/or SEQ ID NO 5706 and/or SEQ ID NO 5734, and more preferably wherein the assay comprises determining percent methylated reference for the sequence defined by the specified SEQ ID NO, no treatment is administered to the individual;
20. less than about 7.000, and preferably wherein the step of determining the methylation status of the one or more CpGs in the panel comprises determining each CpG within SEQ ID NO 5780 and/or SEQ ID NO 5724 and/or SEQ ID NO 5752, and more preferably wherein the assay comprises determining percent methylated reference for the sequence defined by the specified SEQ ID NO, no treatment is administered to the individual;
21. less than about 6.000, and preferably wherein the step of determining the methylation status of the one or more CpGs in the panel comprises determining each CpG within SEQ ID NO 5776 and/or SEQ ID NO 5720 and/or SEQ ID NO 5748, and more preferably wherein the assay comprises determining percent methylated reference for the sequence defined by the specified SEQ ID NO, no treatment is administered to the individual;
22. less than about 5.000, and preferably wherein the step of determining the methylation status of the one or more CpGs in the panel comprises determining each CpG within SEQ ID NO 5783 and/or SEQ ID NO 5727 and/or SEQ ID NO 5755, and more preferably wherein the assay comprises determining percent methylated reference for the sequence defined by the specified SEQ ID NO, no treatment is administered to the individual;
23. less than about 6.000, and preferably wherein the step of determining the methylation status of the one or more CpGs in the panel comprises determining each CpG within SEQ ID NO 5782 and/or SEQ ID NO 5726 and/or SEQ ID NO 5754, and more preferably wherein the assay comprises determining percent methylated reference for the sequence defined by the specified SEQ ID NO, no treatment is administered to the individual;
24. less than about 6.000, and preferably wherein the step of determining the methylation status of the one or more CpGs in the panel comprises determining each CpG within SEQ ID NO 5784 and/or SEQ ID NO 5728 and/or SEQ ID NO 5756, and more preferably wherein the assay comprises determining percent methylated reference for the sequence defined by the specified SEQ ID NO, no treatment is administered to the individual;
25. less than about 5.000, and preferably wherein the step of determining the methylation status of the one or more CpGs in the panel comprises determining each CpG within SEQ ID NO 5777 and/or SEQ ID NO 5721 and/or SEQ ID NO 5749, and more preferably wherein the assay comprises determining percent methylated reference for the sequence defined by the specified SEQ ID NO, no treatment is administered to the individual;
26. less than about 2.000, and preferably wherein the step of determining the methylation status of the one or more CpGs in the panel comprises determining each CpG within SEQ ID NO 5786 and/or SEQ ID NO 5730 and/or SEQ ID NO 5758, and more preferably wherein the assay comprises determining percent methylated reference for the sequence defined by the specified SEQ ID NO, no treatment is administered to the individual;
27. less than about 7.000, and preferably wherein the step of determining the methylation status of the one or more CpGs in the panel comprises determining each CpG within SEQ ID NO 5775 and/or SEQ ID NO 5719 and/or SEQ ID NO 5747, and more preferably wherein the assay comprises determining percent methylated reference for the sequence defined by the specified SEQ ID NO, no treatment is administered to the individual; or
28. less than about 3.000, and preferably wherein the step of determining the methylation status of the one or more CpGs in the panel comprises determining each CpG within SEQ ID NO 5770 and/or SEQ ID NO 5714 and/or SEQ ID NO 5742, and more preferably wherein the assay comprises determining percent methylated reference for the sequence defined by the specified SEQ ID NO, no treatment is administered to the individual.

### Methods of Monitoring

The invention also provides methods of monitoring the presence, or risk of the presence or development of CIN3 and/or cancer in an individual.

“Monitoring” in the context of the present invention may refer to longitudinal assessment of an individual's CIN3 and/or cancer status, risk of harbouring CIN3 and/or cancer or risk of CIN3 and/or cancer development. This longitudinal assessment may be carried out according to any of the assays of the invention described herein. This longitudinal assessment may involve performance of any of the assays of the invention described herein to predict the presence or development of CIN3 and/or cancer in an individual at more than one time point over the course of an undetermined time window. The time window may be any period of time whilst the individual is still living. The time window may persist for the lifetime of the individual. The time window may persist until the individual's CIN3 and/or cancer status, risk of harbouring CIN3 and/or cancer or risk of CIN3 and/or cancer development falls below a certain level. The level may be a particular cancer index value.

The invention thus encompasses a method of monitoring for the presence, absence or development of CIN3 and/or cancer, particularly cervical or endometrial cancer, most preferably cervical cancer, particularly cervical and/or endometrial cancer, most preferably cervical cancer, in an individual, the method comprising:
a. assessing the presence or absence of CIN3 and/or cancer in an individual or assessing cancer development in an individual to establish a CIN3 and/or cancer status for the individual by performing any one of the assays of the invention described herein at a first time point;
b. assessing the presence or absence of CIN3 and/or cancer in the individual or assessing CIN3 and/or cancer development in the individual to establish a CIN3 and/or cancer status for the individual by performing any one of the assays of the invention described herein at one or more further time points, preferably wherein the CIN3 and/or cancer status of the individual in steps a and b are assessed using the same assay; and
c. monitoring any change in the CIN3 and/or cancer status of the individual between time points.

The invention also encompasses a method of monitoring for the presence, absence or development of CIN3 and/or cancer, particularly cervical or endometrial cancer, most preferably cervical cancer, particularly cervical and/or endometrial cancer, in an individual, the method comprising:
a. assessing the presence or absence of CIN3 and/or cancer in an individual or assessing cancer development in an individual to establish a CIN3 and/or cancer status for the individual by performing an assay at a first time point, comprising:
   1. providing a sample which has been taken from the individual, the sample comprising a population of DNA molecules;
   2. determining in the population of DNA molecules in the sample the methylation status of a panel of:
      a. one or more CpGs selected from a panel of CpGs identified in SEQ ID NOs 1 to 5000 wherein the CpGs are identified at nucleotide positions 61 to 62; and/or
      b. one or more CpGs selected from within a panel of one or more Differentially Methylated Regions (DMRs) defined by SEQ ID NOs 5001 to 5418, wherein the CpGs are denoted by CG;
   3. deriving a cancer index value based on the methylation status of the one or more CpGs in the panel; and
   4. assessing the presence, absence or development of CIN3 and/or cancer, particularly cervical or endometrial cancer, most preferably cervical cancer, in the individual based on the cancer index value, wherein the assay is characterised as having an area under the curve (AUC) of 0.60 or more as determined by receiver operating characteristics (ROC);
b. assessing the presence or absence of CIN3 and/or cancer in the individual or assessing cancer development in the individual to establish a CIN3 and/or cancer status for the individual by performing the assay of steps a(1) to a(4) or by performing any one of the assays of the invention described herein at one or more further time points; and
c. monitoring any change in the CIN3 and/or cancer status of the individual between time points.

In any of the methods of monitoring described herein, the steps of assessing the presence, absence or development of CIN3 and/or cancer, particularly cervical or endometrial cancer, most preferably cervical cancer, in an individual based on a cancer index value may involve the application of threshold values. Threshold values can provide an indication of an individual's CIN3 and/or cancer status, risk of having CIN3 and/or cancer or an individual's risk of CIN3 and/or cancer development. For example, cancer index values may indicate the presence or absence of CIN3 and/or cancer, or a high or low risk of having or developing CIN3 and/or cancer. In any of the methods of monitoring encompassed by the invention, the step of predicting the presence, absence or development of CIN3 and/or cancer, particularly cervical or endometrial cancer, most preferably cervical cancer, in an individual involves deriving a cancer index value.

The invention further encompasses a method of measuring methylation in a patient at multiple time points comprising (a) assessing the presence, absence or development of CIN3 and/or cancer in an individual by performing any one of the assays of the invention described herein at a first time point; (b) assessing the presence, absence or development of CIN3 and/or cancer in the individual by performing any one of the assays of the invention described herein at one or more further time points, and (c) detecting differential methylation status between (a) and (b).

In any of the methods of monitoring described herein, depending on the risk of the presence or development of CIN3 and/or cancer in the individual, one or more treatments are administered to the individual according to any one of the methods of treatment encompassed by the invention and described herein, or wherein the cancer index value of the individual is less than about −0.530, and preferably wherein the assay comprises determining methylation β-values for each CpG in the panel of one or more CpGs, no treatment is administered to the individual. Different treatments may be administered depending on the stratification of an individual on the basis of their CIN3 and/or cancer status, risk of harbouring CIN3 and/or cancer or on the basis of their risk of CIN3 and/or cancer development. The method may further comprise administration of one or more treatments according to the methods of treatment described herein.

The cancer index value may change between any two or more time points. For this reason, longitudinal monitoring of an individual's cancer index value could be of particular benefit to the assessment of, for example, cancer progression, prevention of recurrence of cancer, cancer treatment efficacy, or cancer efficacy.

In any of the methods of monitoring described herein, the one or more further time points may be any suitable time point. Preferably the one or more further time points may of suitable distance apart for sufficiently frequent screening in order to predict any particularly early onset cases of presence or development of cancer in an individual. Preferably the one or more further time points may be of suitable distance apart for assessing the efficacy of one or more treatments. Preferably the one or more further time points may be of suitable distance apart for predicting whether an individual remains free of cancer after a successful course of treatment. The one or more further time points may be about monthly, about two monthly, about three monthly, about four monthly, about five monthly, about six monthly, about seven monthly, about eight monthly, about nine monthly, about ten monthly, about eleven monthly, about yearly, about two yearly, or more than two yearly.

In any of the methods of monitoring described herein, changes may be made to the one or more treatments wherein a positive or negative responses to the one or more treatments are observed. Treatments may be changed in accordance with the methods of treatments described herein. Treatments may particularly be changed if the individual's cancer status or risk stratification, based on their cancer index value, changes.

In any of the methods of monitoring encompassed by the invention, the step of predicting the presence or development of CIN3 and/or cancer in an individual may involve the use of any one of the arrays described herein.

### Biological Samples

The assays described herein are preferably performed on DNA from cells derived from/obtained from samples from tissue in which the native tissue structure is preserved e.g. a biopsy, or a sample comprising exfoliated cells from a tissue surface. The samples may comprise epithelial cells. The sample may particularly be derived from the cervix, the vagina, the buccal area, blood and/or urine. The sample is preferably a cervical liquid-based cytology sample, and more preferably a cervical smear sample.

Preferably, any one of the assays described herein for assessing the presence, absence or development of CIN3 and/or cancer, particularly cervical or endometrial cancer, most preferably cervical cancer, in an individual comprises providing a sample which has been taken from the individual. Preferably the individual is a woman.

In any of the assays described herein, the assay may or may not encompass the step of obtaining the sample from the individual. In assays which do not encompass the step of obtaining the sample from the individual, a sample which has previously been obtained from the individual is provided.

The sample may be provided directly from the individual for analysis or may be derived from stored material, e.g. frozen, preserved, fixed or cryopreserved material.

In any of the assays described herein, the sample may be self-collected or collected by any suitable medical professional.

Any of the assays described herein, the sample may comprise cells. The sample may comprise genetic material such as DNA and/or RNA.

Any of the assays described herein may involve providing a biological sample from the patient as the source of patient DNA for methylation analysis.

Any of the assays described herein may involve obtaining patient DNA from a biological sample which has previously been obtained from the patient.

Any of the assays described herein may involve obtaining a biological sample from the patient as the source of patient DNA for methylation analysis. The sample may be self-collected or collected by any suitable medical professional. Procedures for obtaining a biological sample include biopsy.

Methods for sample isolation and for the subsequent extraction and isolation of DNA from such cell or tissue samples in preparation for assessing DNA methylation, are well known to those skilled in the art. In the context of the assays or methods described herein, the entirety of a sample may be used, or alternatively cells may be concentrated or cell types may be fractionated in order to only apply subsets of one or more cell types to the present assays or methods. Any suitable methods of concentration or fractionation may be used.

### Types of Cancers

The methods described herein may be applied to any cancer. Preferably, the methods described herein may be applied to cervical cancer and/or endometrial cancer, particularly cervical cancer. The methods described herein are most preferably applied to cervical cancer.

The cancer may be a primary cancer lesion. The cancer may be a secondary cancer lesion. The cancer may be a metastatic lesion.

In assays described herein, wherein the assay is for assessing the presence, absence or development of grade 3 cervical epithelial neoplasia (CIN3) and/or cervical cancer, the cervical cancer may preferably be a squamous cell cancer, an adenocarcinoma or an adenosquamous carcinoma. Any of the assays described herein may additionally, or alternatively, be for assessing the presence, absence or development of endometrial cancer.

In assays described herein, wherein the assay is for assessing the presence, absence or development of endometrial cancer, the endometrial cancer may preferably be endometroid cancer, uterine carcinosarcoma, squamous cell carcinoma, small cell carcinoma, transitional carcinoma, serous carcinoma, clear-cell carcinoma, mucinous adenocarcinoma, undifferentiated carcinoma, dedifferentiated carcinoma or serous adenocarcinoma.

### Arrays and Kits

The invention also encompasses arrays capable of discriminating between methylated and non-methylated forms of CpGs as defined herein; the arrays may comprise oligonucleotide probes specific for methylated forms of CpGs as defined herein and oligonucleotide probes specific for non-methylated forms of CpGs as defined herein.

In any of the arrays described herein, the array may comprise oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 5418.

The panel may consist of at least 500 CpGs selected from the CpGs identified in SEQ ID NOs 1 to 5418, preferably wherein the CpGs comprise the CpGs identified in SEQ ID NOs 1 to 500.

The panel may consist of at least 1000 CpGs selected from the CpGs identified in SEQ ID NOs 1 to 5418, preferably wherein the CpGs comprise the CpGs identified in SEQ ID NOs 1 to 1000.

The panel may consist of at least 1500 CpGs selected from the CpGs identified in SEQ ID NOs 1 to 5418, preferably wherein the CpGs comprise the CpGs identified in SEQ ID NOs 1 to 1500.

The panel may consist of at least 2000 CpGs selected from the CpGs identified in SEQ ID NOs 1 to 5418, preferably wherein the CpGs comprise the CpGs identified in SEQ ID NOs 1 to 2000.

The panel may consist of at least 2500 CpGs selected from the CpGs identified in SEQ ID NOs 1 to 5418, preferably wherein the CpGs comprise the CpGs identified in SEQ ID NOs 1 to 2500.

The panel may consist of at least 3000 CpGs selected from the CpGs identified in SEQ ID NOs 1 to 5418, preferably wherein the CpGs comprise the CpGs identified in SEQ ID NOs 1 to 3000.

The panel may consist of at least 3500 CpGs selected from the CpGs identified in SEQ ID NOs 1 to 5418, preferably wherein the CpGs comprise the CpGs identified in SEQ ID NOs 1 to 3500.

The panel may consist of at least 4000 CpGs selected from the CpGs identified in SEQ ID NOs 1 to 5418, preferably wherein the CpGs comprise the CpGs identified in SEQ ID NOs 1 to 4000.

The panel may consist of at least 4500 CpGs selected from the CpGs identified in SEQ ID NOs 1 to 5418, preferably wherein the CpGs comprise the CpGs identified in SEQ ID NOs 1 to 4500.

The panel may consist of at least 5000 CpGs selected from the CpGs identified in SEQ ID NOs 1 to 5418, preferably wherein the CpGs are the CpGs identified in SEQ ID NOs 1 to 5000.

The panel may consist of all CpGs identified in SEQ ID NOs 1 to 5000 and identified at nucleotide positions 61 to 62, and identified in SEQ ID NOs 5001 to 5418 and denoted by CG.

In some embodiments the array is not an Infinium MethylationEPIC BeadChip array or an Illumina Infinium HumanMethylation450 BeadChip array.

Separately or additionally, in some embodiments the number of CpG-specific oligonucleotide probes of the array is 482,000 or less, 480,000 or less, 450,000 or less, 440,000 or less, 430,000 or less, 420,000 or less, 410,000 or less, or 400,000 or less, 375,000 or less, 350,000 or less, 325,000 or less, 300,000 or less, 275,000 or less, 250,000 or less, 225,000 or less, 200,000 or less, 175,000 or less, 150,000 or less, 125,000 or less, 100,000 or less, 75,000 or less, 50,000 or less, 45,000 or less, 40,000 or less, 35,000 or less, 30,000 or less, 25,000 or less, 20,000 or less, 15,000 or less, 10,000 or less, 5,000 or less, 4,000 or less, 3,000 or less or 2,000 or less.

The CpG panel may comprise any set of CpGs defined in the assays of the invention described herein.

The arrays of the invention may comprise one or more oligonucleotides comprising any set of CpGs defined in the assays of the invention, wherein the one or more oligonucleotides are hybridized to corresponding oligonucleotide probes of the array.

The invention also encompasses a process for making a hybridized array described herein, comprising contacting an array according to the present invention with a group of oligonucleotides comprising any set of CpGs defined in the assays of the invention.

Any of the arrays as defined herein may be comprised in a kit. The kit may comprise any array as defined herein together with instructions for use.

The invention further encompasses the use of any of the arrays as defined herein in any of the assays for determining the methylation status of CpGs for the purposes of predicting the presence or development of cancer in an individual.

The following Examples serve to illustrate but not to limit the invention.

### Examples

In the Examples described herein, WID-CIN-Index is a cancer index value wherein the index value has been determined by assaying in a population of DNA molecules derived from a given sample from an individual the methylation status of a panel of CpGs selected from the CpGs identified in SEQ ID NOs 1 to 5000 and/or within Differentially Methylated Regions defined by SEQ ID NOs 5001 to 5418.

In some instances within the Examples, all CpGs defined by SEQ ID NOs: 1 to 5000 have been included in the panel which has been assayed to obtain a cancer index value. In addition, specific sub-selections of CpGs from among the 500 CpGs defined by SEQ ID NOs: 1 to 500 have been included in the panel which has been assayed to obtain a cancer index value. In these instances, the cancer index value's ability to discriminate between CIN3 and/or cancer positive and CIN3 and/or cancer negative women is described, wherein discriminatory ability of the index is characterised by AUC and received operating characteristics.

Cervical screening is currently transitioning from cytology to HPV-testing. This will lead to increased sensitivity but decreased specificity in mass screening. An objective, automatable test that can accurately triage HPV+ve women independently of sample heterogeneity and age, while also capable of detecting other epithelial uterine cancers is urgently required. The inventors, along with other scientists, have shown the feasibility of utilising DNA methylation markers to identify women with pre-invasive or invasive cancers. The clinical use of DNA methylation markers to identify women at high risk for CIN3+ has been hindered by several factors:
(i) A suboptimal sensitivity in detecting CIN3 or CIN 3+, particularly in young women below 30 who have a substantially higher prevalence of HPV and thus have an increased need for high performance triage testing. For instance, the GynTect test (which utilises DNA methylation of six genes) has a sensitivity for CIN3 at 35% in <30 year old and 76% in ≥30 year old women and the sensitivity in detecting CIN3+ for the QIAsure (which uses methylation of two genes) at 37.5% in <30 year old and 89.3% in ≥30 year old women, who were referred with an abnormal cytology.
(ii) A low specificity, which is also age dependent and comparable to cytology which ranges from 76.6% in ≥30 year old women to 87.8% in <30 year old women.
(iii) A lack of data prevents judgement as to whether a DNA methylation marker or marker panel is capable of identifying HPV+ve women which, despite being cytology-ye at the time of assessment, go on to develop CIN3+ in succeeding years.

Using a cohort-based nested case-control setting, the inventors aimed to develop and validate a DNA methylation signature (called Women's cancer risk IDentification CIN3 index, WID-CIN3-index) in cervical liquid-based cytology samples. The cancer index value should be capable of both diagnosing and predicting the future risk of CIN3+.

### Materials and Methods

### Cervical Liquid-based Cytology Sample Collection

All cervical liquid-based cytology samples processed in the capital region of Stockholm in Sweden are biobanked through a state-of-the-art platform at the Karolinska University Laboratory, Karolinska University Hospital, as previously described (Perskvist et al, 2013). Since the year 2013, virtually 100% of the ˜150 000 LBCs per year are compacted and stored in a 600 microliter, 96 well plate format at −27°. This allows for preservation of intact cells, and analyses of DNA, RNA and protein content, among others. The biobank is linked to the Swedish health register infrastructure through the individually unique personal identification number (PIN) (Ludvigsson et al, 2016).

The inventors defined a cohort of all women participating in cervical screening, or clinically indicated testing, during the years 2013-2015 and linked this to the National Cancer Register at the Swedish National Board of Health and Welfare, to identify all cases of CIN3 or invasive cervical cancer (CIN3+) occurring in the sample collection during these years (n=samples from n women). The inventors utilized this cohort to identify a) all women with prevalent CIN3+, b) all women with a normal sample which was succeeded by a later diagnosis of CIN3+ within 1-4 years, c) all women with low-grade lesions of the cervix, so-called CIN1-2 and d) an age- and calendar-year of sample frequency-matched control group of healthy women who had no record of cervical lesions in the National Cancer Register ever. During the years 2013-2015, some groups of the population had been randomized to HPV screening and some to primary cytology. The inventors carefully balanced each sample set to reflect this fact. All samples which did not have HPV results on record were put through high-performance HPV-testing on the cobas48000 assay (ref), which had been the publicly tendered HPV testing platform during this entire period. The inventors further linked all samples to comprehensive, harmonized records of their cytology diagnosis held in the National Cervical Screening Register (NKCx.se). This enabled stratification of all samples by high-risk HPV positivity, and cytology status, respectively.

To maximize DNA content, we, blinded to case-control status, visually screened all eligible vials of biobanked samples to ensure that a visible cell pellet was present. Approximately ⅓ of samples had such a pellet and this was independent of case-control or CIN3/ICC status. The inventors then aliquoted 100 microliter from each sample for UCL to perform methylation analyses.

### Sample Processing and DNA Extraction

650 ul of PBS was added to each 100 ul cervical liquid-based cytology sample received from the Karolinska University Laboratory biobank and centrifuged for 15 mins at 4,600 rpm. The supernatant was carefully removed and the pellet was washed with a further 750 ul PBS. The samples were then vortexed and centrifuged again for 15 mins at 4,600 rpm. After careful removal of the second PBS wash the samples the inventors resuspended in lysis buffer from the Nucleo-Mag Blood 200 ul kit (Macherey Nagel, cat #744501.4) which was used in conjunction with the Hamilton Star liquid handling platform for high throughout DNA extraction. DNA concentration and quality absorbance ratios were measured using Nanodrop-8000, Thermoscientific Inc. Extracted DNA was stored at −80° C. until further analysis.

### DNA Methylation Array Analysis

Cervical was normalised to 10-25 ng/ul and 200-500 ng total DNA was bisulfite modified using the EZ-96 DNA Methylation-Lightning kit (Zymo Research Corp, cat #D5047) on the Hamilton Star Liquid handling platform. 8 ul of modified DNA was subjected to methylation analysis on the Illumina InfiniumMethylation EPIC BeadChip (Illumina, CA, USA) at UCL Genomics according to the manufacturer's standard protocol.

### Methylation Analysis

All methylation microarray data were processed through the same standardised pipeline. Raw data was loaded using the R package minfi. Any samples with median methylated and unmethylated intensities <9.5 were removed. Any probes with a detection p-value >0.01 were regarded as failed. Any samples with >10% failed probes, and any probes with >10% failure rate were removed from the dataset. Beta values from failed probes (approximately 0.001% of the dataset) were imputed using the impute.knn function as part of the impute R package.

Non-CpG probes (2,932), SNP-related probes as identified by Zhou et. al. (82,108), and chrY probes were removed from the dataset. An additional 6,102 previously identified probes that followed a trimodal methylation pattern characteristic of an underlying SNP were removed. Background intensity correction and dye bias correction was performed using the minfi single sample preprocessNoob function. Probe bias correction was performed using the beta mixture quantile normalisation (BMIQ) algorithm.

The fraction of immune cell contamination, and the relative proportions of different immune cell subtypes in each sample, were estimated using the EpiDISH algorithm using the epithelial, fibroblast and immune cell reference dataset. The top 1,000 most variable probes (ranked by standard deviation) were used in a principal component analysis. Statistical tests were performed in order to identify any anomalous associations between plate, sentrix position, date of array processing, date of DNA creation, study centre, immune contamination fraction, age, type (case versus control) and the top ten principal components. No anomalous associations were found.

### Methylight Reaction Design

Two ranked lists of CpGs were generated. The first was ranked according to the epithelial delta-beta estimates (the estimated difference in methylation between cases and controls in cervical epithelial cells). The second was ranked according to p-values (derived from a linear model comparing cases to controls after adjustment for immune cell proportion and age). For each CpG we identified any contiguous CpGs within +/−500 bp. The inventors computed and plotted the mean methylation in cases and controls across all CpGs within this 1000 bp region. Upon visual inspection of the top 50 CpGs (in both ranked lists) we identified a number of candidate regions according to the following criteria:
1. Regions where the methylation level was close to zero in healthy controls.
2. Regions where the variability within controls was relatively low.
3. Regions where the methylation level was elevated in cases.
4. Regions that included two or more CpG sites

### Statistical Analyses for Classifier Development

Contamination by immune cells presented a challenge with respect to the identification of differentially methylated positions (DMPs) as differential methylation that occurred solely in epithelial cells was diminished in samples with high IC and vice versa. In order to overcome this, the inventors linearly regressed the beta values on IC for each CpG site, the linear models being fitted to cases and controls separately. The intercept points at IC=0 were used as estimates of mean beta values in cases and controls in a pure epithelial cell population. The difference between these intercept points provided a delta-beta estimate in epithelial cells. The difference between intercept points at IC=1 provided immune cell delta-beta estimates.

The R package glmnet was used to train classifiers with a mixing parameter value of alpha=0 (ridge penalty) and alpha=1 (lasso penalty) with binomial response type. Data from the discovery set dataset were used to fit the classifiers. A ranked list of CpGs was generated by taking the CpG with the largest epithelial delta-beta, followed by the CpG with the largest immune delta-beta, followed by the next largest epithelial delta-beta and so forth (any duplicates were removed). The top n CpGs from the list of ranked CpGs were used as inputs to the classifier. Ten-fold cross-validation was used inside the training set by the cv.glmnet function in order to determine the optimal value of the regularisation parameter lambda. The AUC was used as a metric of classifier performance. Out-of-bag AUC estimates (based on the cross validation folds which were not used for training the classifier) were as a function of n, the number of CpGs used as inputs during training. The maximum value of n was 10,000.

The optimal classifier was selected based on the highest out-of-bag AUC obtained on the discovery set. Once the classifier was finalised it was then applied to the validation datasets.

Denoting the top n CpGs as β₁, . . . , βₙ and the regression coefficients from the trained classifier as w₁, . . . , wₙ then WID-CIN-index=Σᵢ₌₁ⁿ(wᵢβᵢ−μ)/σ where μ and a are defined as the mean and standard deviation of the quantity Σᵢ₌₁ⁿwᵢβᵢ in the discovery set (that is, the index is scaled to have zero mean and unit standard deviation in the discovery set). The island (open sea) subcomponent was obtained by restricting the above sum to CpGs located in CpG islands (open seas) as defined in the Illumina manifest version B4.

### Example

### Sample Heterogeneity and Differential Methylation

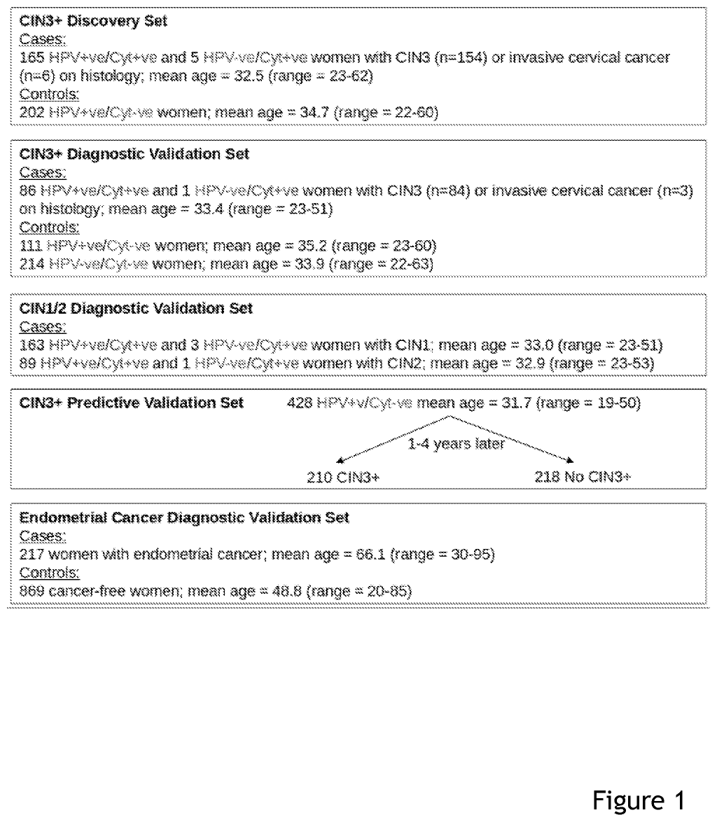

For the Discovery Set (FIG. 1), the inventors identified cervical liquid-based cytology samples from 169 cytology+ve women (164 HPV+ve and 5 HPV-ve) at the time of cervical intraepithelial neoplasia (CIN) grade 3 (n=154) or invasive cancer (n=6) diagnosis (CIN3+) and age- and calendar year-matched HPV+ve cytology-ye women (n=202) from the Karolinska Institute population-based cervical cytology biobank.

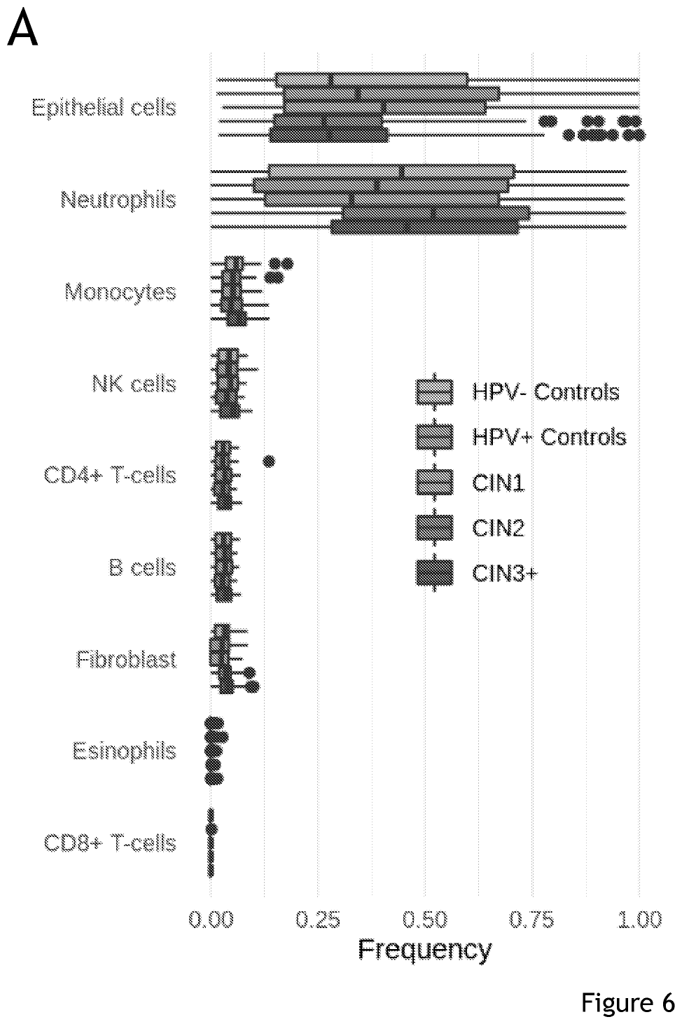

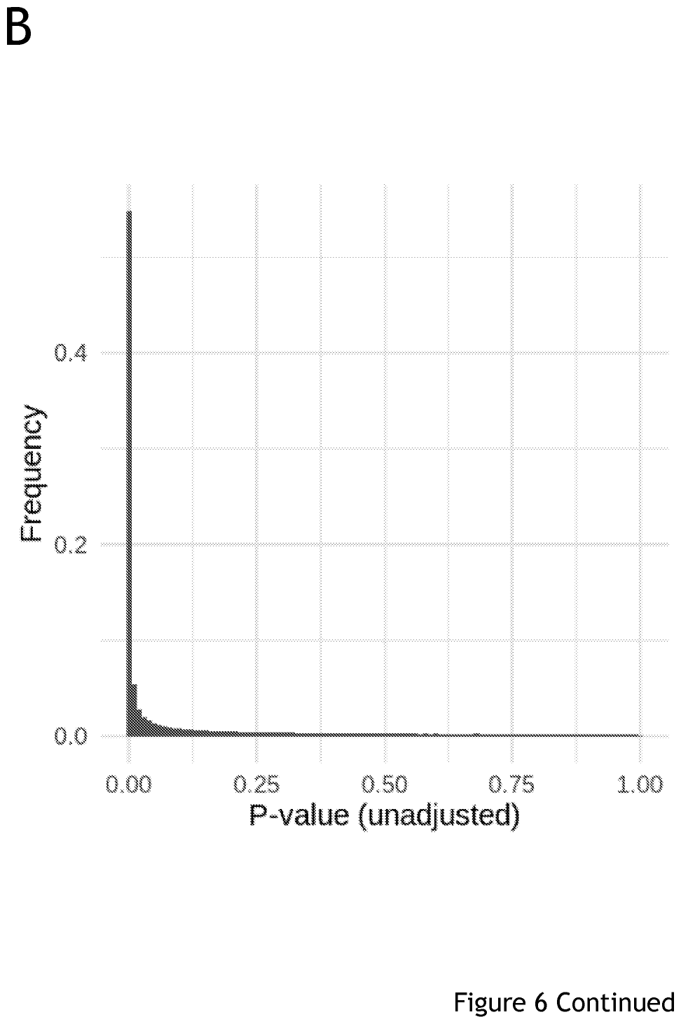

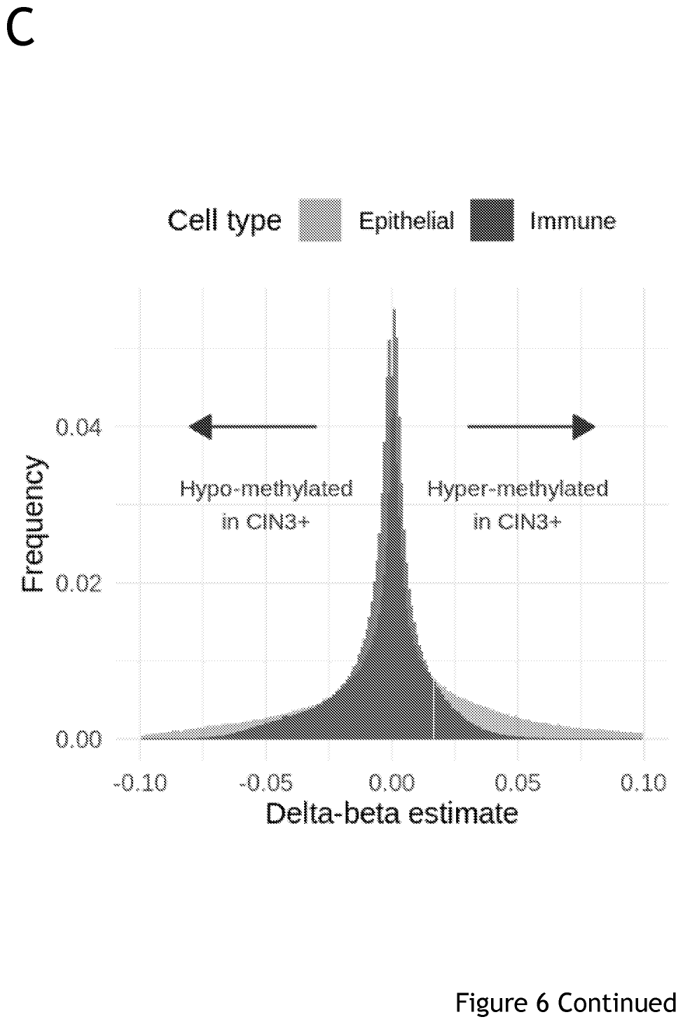

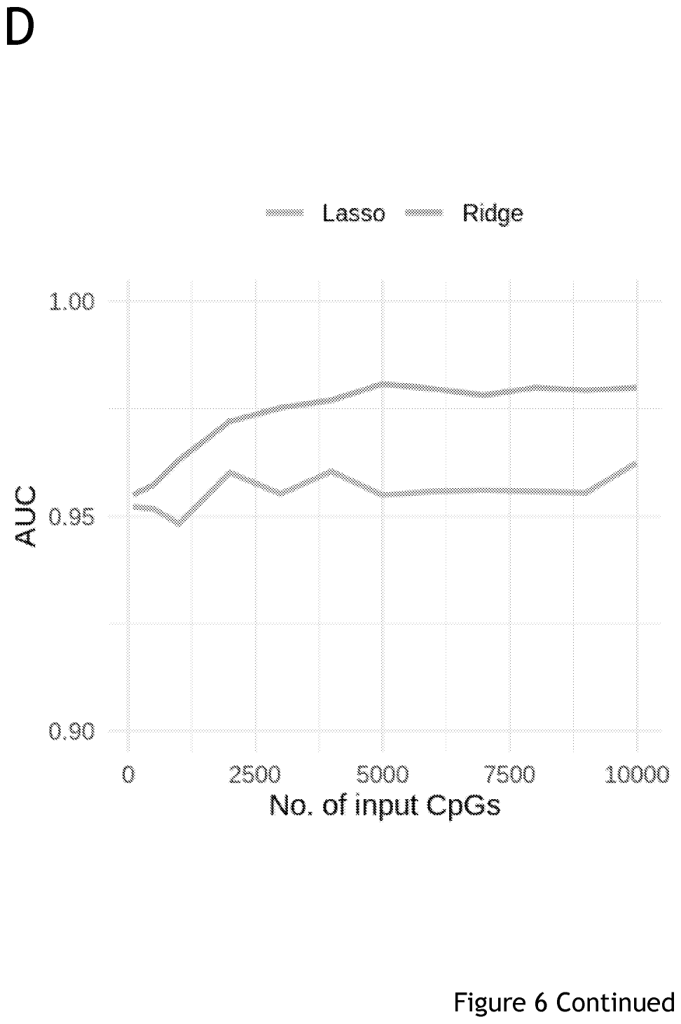

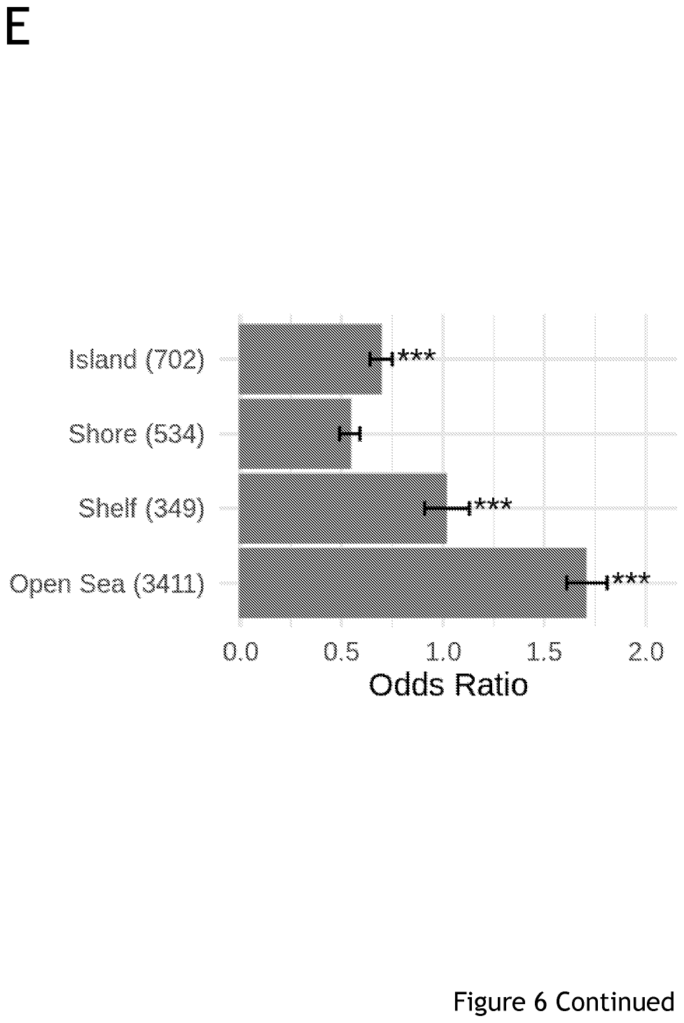

Previously the inventors found that methylation differences may vary due to immune cell type composition in cases compared to controls. Hence, the inventors assessed the level of cell type heterogeneity in each cervical cytology sample using EpiDISH, an algorithm that infers the relative proportion of epithelial cells, fibroblasts, and seven subtypes of immune cells in each sample. The cell-type distributions were broadly similar between CIN3+ cases and controls with an increase in immune cells in CIN2 and CIN3+ cases (FIG. 6a).

Assessing 850,000 CpG sites after false discovery rate adjustment the inventors found 158,434 CpGs to be significantly differentially methylated between CIN3+ cases and controls (FIG. 6b) with the main difference based on epithelial cells and with a skew towards hypermethylation in CIN3+ cases (FIG. 6c).

### Development of Discriminatory Index

In order to derive a diagnostic methylation signature to detect CIN3 or invasive cervical cancer, termed the WID-CIN-index, the inventors used ridge and lasso classifiers in the Discovery Set to classify individuals as CIN3+ cases or controls. The area under the receiver operator characteristic curve (AUC) was used as a measure of predictive performance. CpGs were ranked according to the delta-beta between CIN3+ cases and controls.

Predictive performance was estimated based on 10-fold cross-validation within the Discovery Set and evaluated as a function of the number of CpGs used to train the classifier. The optimal Discovery Set AUC of 0.98 was achieved using 5,000 CpGs with ridge regression (FIG. 6d). The classifier was developed entirely using the Discovery Set before it was applied to the validation datasets.

The inventors found that the index was enriched for Open Sea regions and depleted for CpG islands (FIG. 6e). Ridge regression combines information from all input CpGs in contrast to lasso regression, which typically selects a small subset of inputs. Ridge regression offered consistently superior performance suggesting that the discriminatory signal was most robustly extracted by combining a large number of signals from multiple CpG sites.

### Validation of the Diagnostic Capacity of the WID-Test

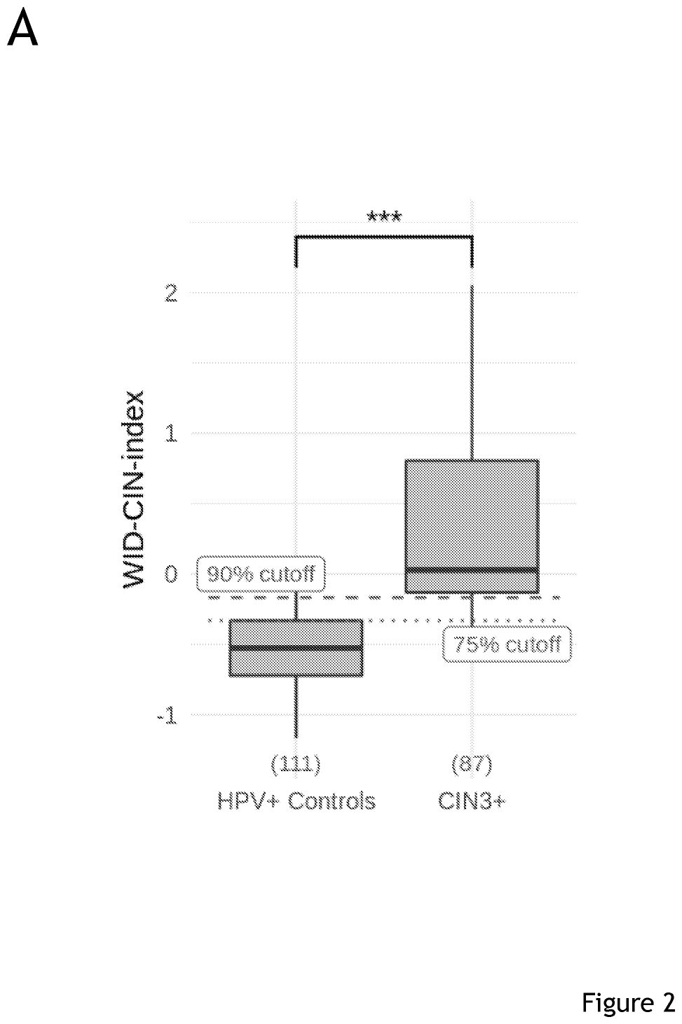

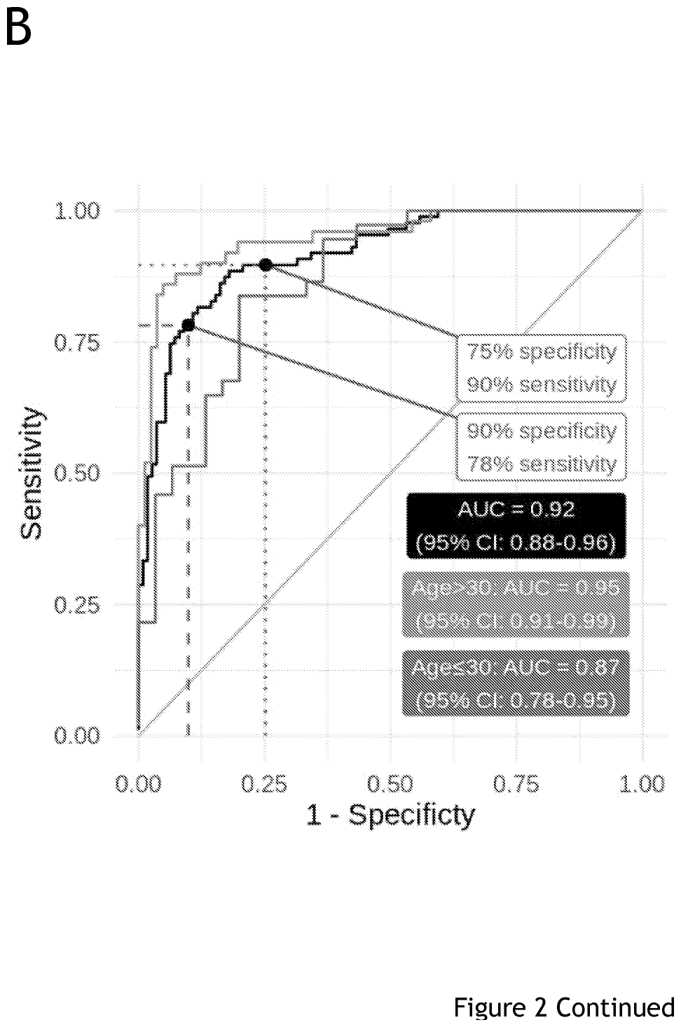

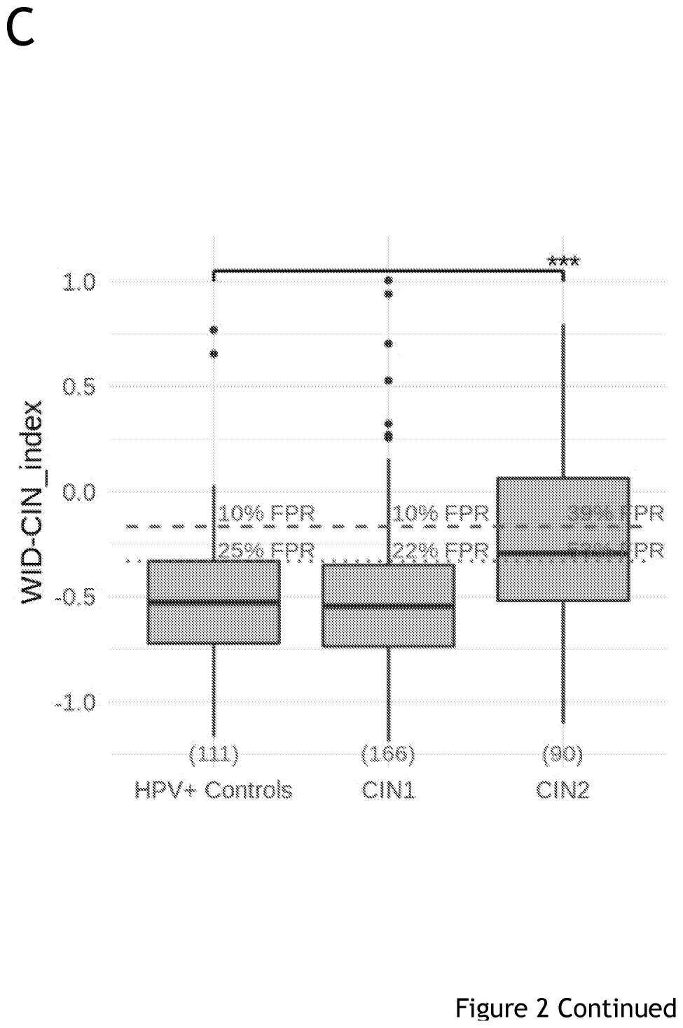

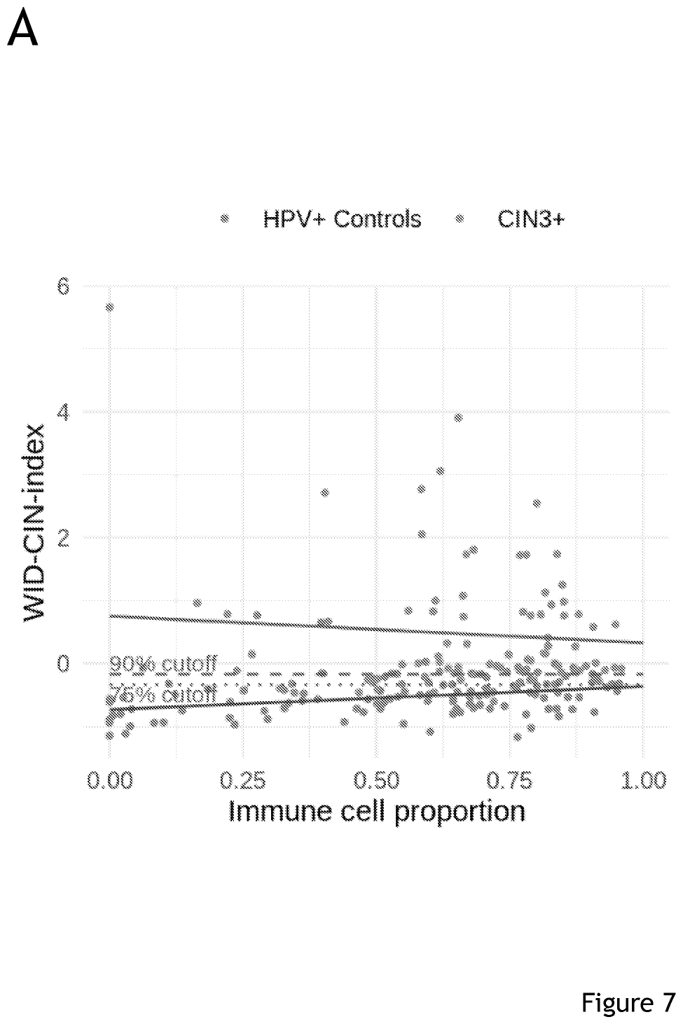

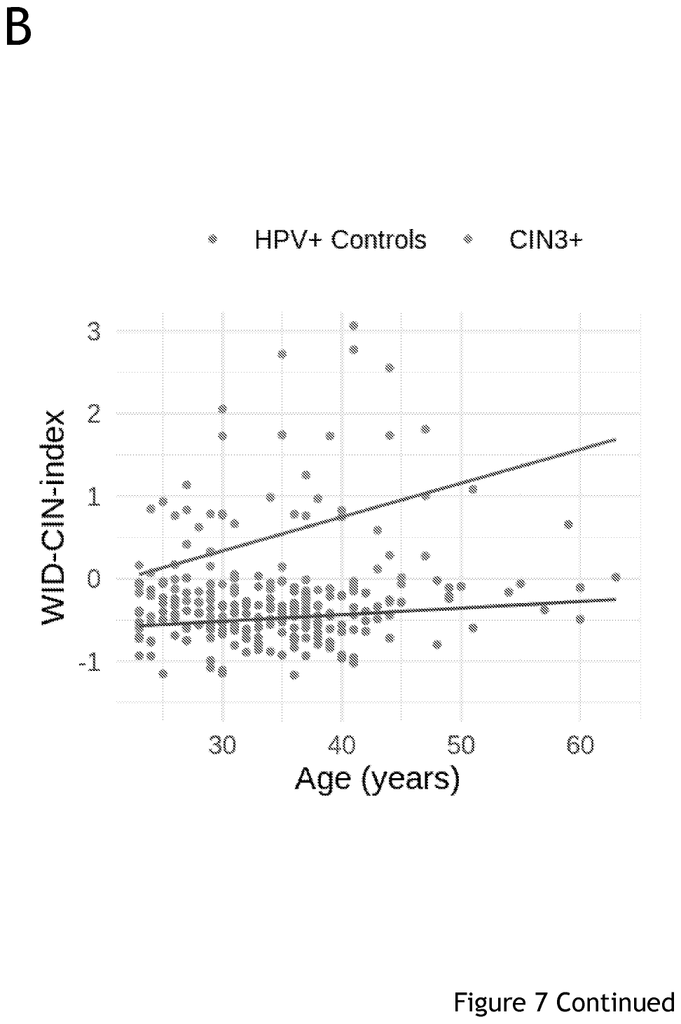

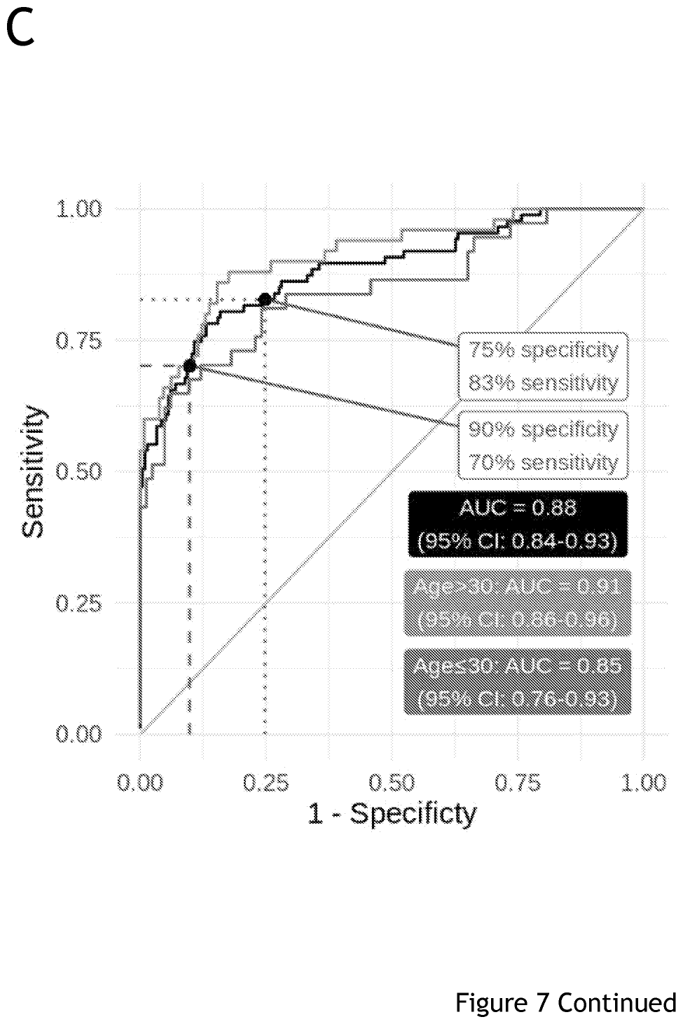

In order to assess the diagnostic capacity of the WID-test the inventors analysed an independent dataset of cervical cytology samples consisting of 87 women who were diagnosed with CIN3+ and 111 HPV+ controls (see FIG. 1). The WID-CIN3-index was computed for each woman (FIG. 2a) resulting in an AUC of 0.92 (95% CI: 0.88-0.96) (FIG. 2b). At 75% and 90% specificity, the sensitivity was 90% and 78%, respectively. Discriminatory performance was independent of immune cell proportion (FIG. 7a). Due to the fact that the WID-CIN3+-index shows an age-dependency (FIG. 7b), the inventors assessed ≤30 and >30 year old women separately. The performance of the WID-CIN test was better in >30 year old women (AUC 0.95; 95% CI 0.91-0.99) compared to women ≤30 years (AUC 0.87; 95% CI 0.78-0.95) (FIG. 2b). The inventors further validated the diagnostic validity of the WID-CIN test in the context of HPV-ve/Cytology-ve controls and received a similar performance (FIG. 7c).

The fact that almost all CIN3+ cases would have been correctly classified (irrespective of the age) at a specificity of 50% (FIG. 2b) suggests a clear superiority of the WID-CIN test over cytology. In order to assess whether the WID-CIN test is also superior when only assessed in Cytology+ve women and whether it is specific for CIN3+ or whether it would also detect HPV+ve/Cytology+ve women who—on colposcopic assessment and cervical biopsy—present with CIN1 or CIN2, the inventors analysed 256 Cytology+ve samples of which 166 and 90 transpired to be CIN1 and CIN2 on biopsy, respectively (FIG. 2c). The inventors applied a cutoff that led to a 100% sensitivity for CIN3+(i.e. −0.60 and −0.56 for <30 and ≥30 year old women, respectively) which led to a true negative rate of 39.5% and 32.8, respectively (FIG. 5a), indicating that up to 40% of Cytology+ve women would have not required a colposcopic assessment or a biopsy. Whereas conventional cytology in HPV+ve women leads to a sensitivity/specificity of 52%/75%, the p16/Ki-67 dual-stained cytology results in a substantially improved performance (75%/74%). Setting the sensitivity to detect CIN3+ cases at 75% and including all HPV+ve women in our diagnostic sets, this led to a specificity of 93.7% overall and to 80% and 97.5% in <30 and ≥30 year old women, respectively (FIG. 5b).

### Validation of the Predictive Capacity of the WID-Test

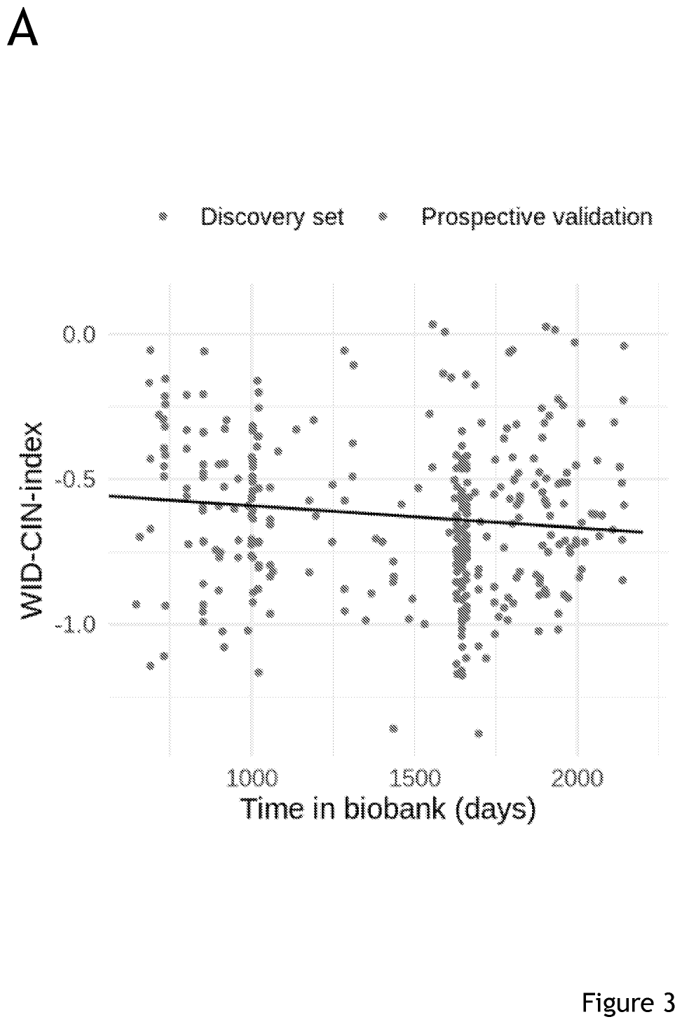

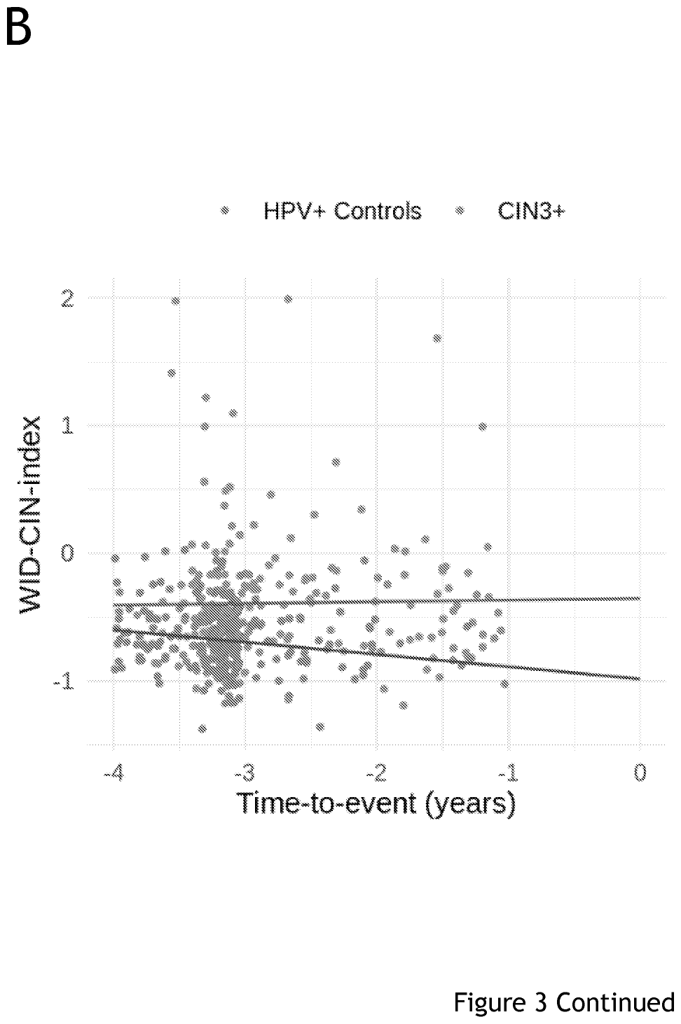

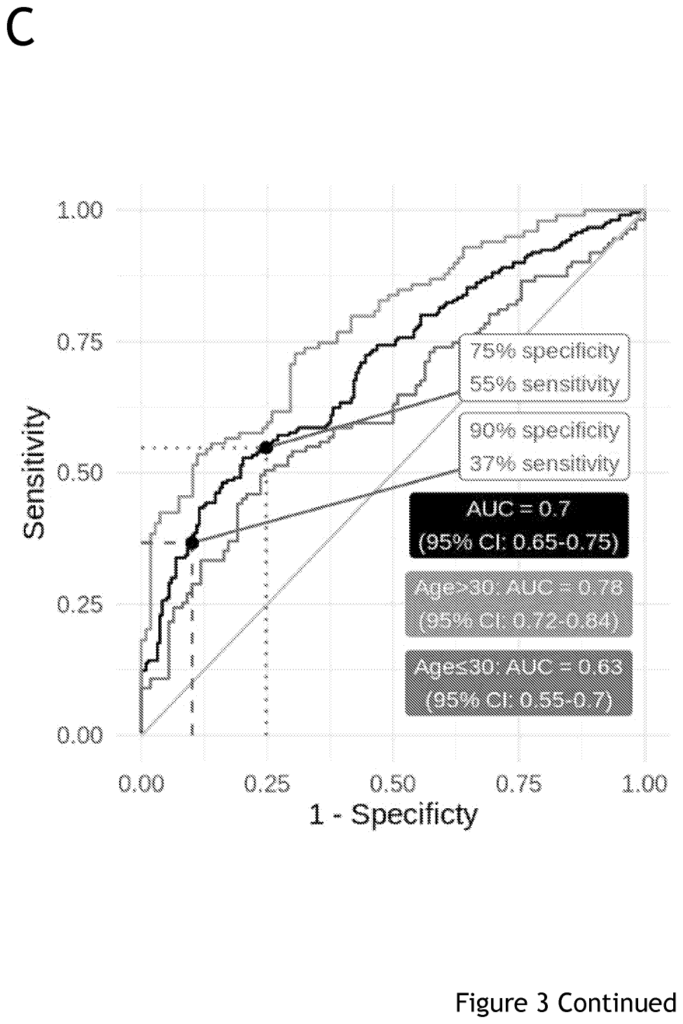

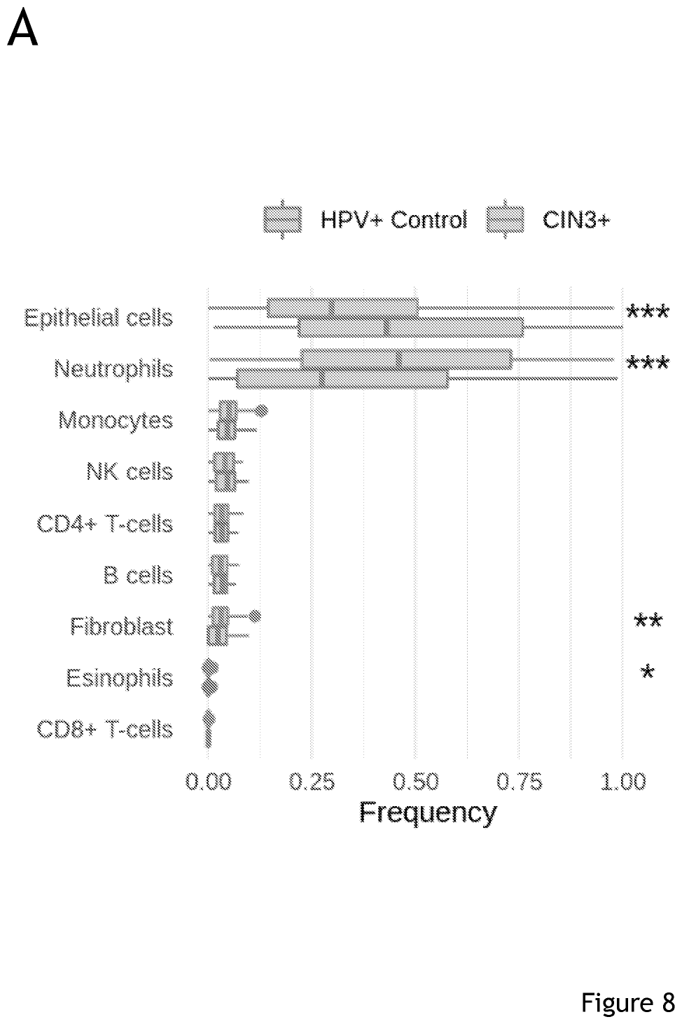

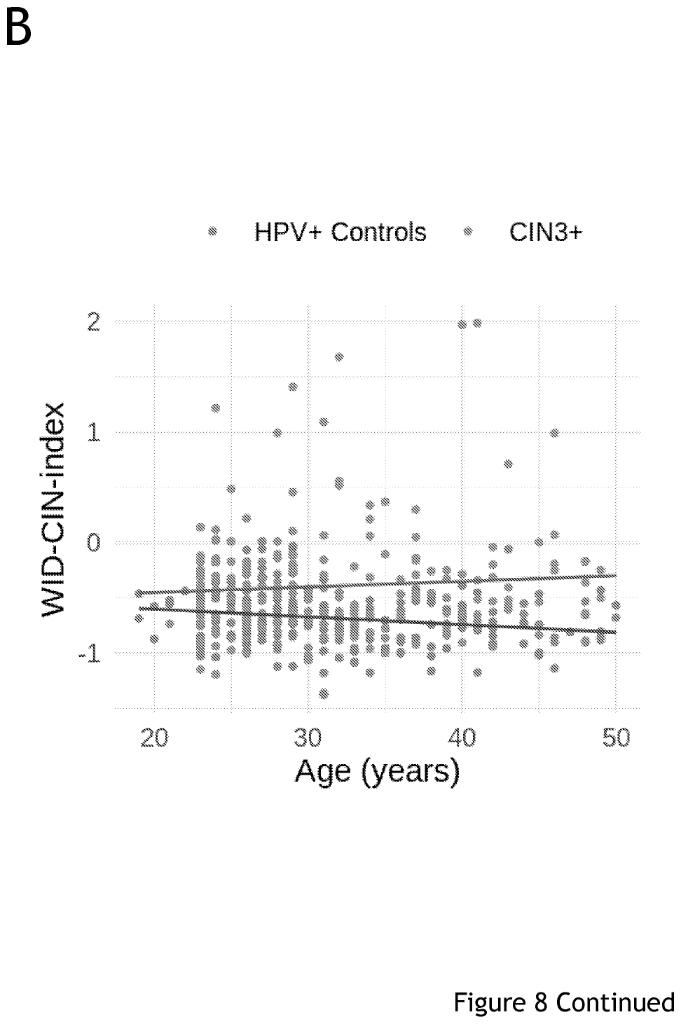

In order to optimise the efficacy of the screening, not only is it important to identify women with current CIN3+ but also those women with the highest risk of developing CIN3+ in the future. In order to address this, the inventors analysed 428 HPV+ve/Cytology-ve women of whom 210 were diagnosed with CIN3+1 to 4 years after they provided their sample and 218 remained disease-free within the same period (FIG. 1). Sample cell-type composition was broadly comparable to the Discovery Set (FIG. 8a). For the observation period to be of a satisfactory duration, the inventors selected samples from women that had provided samples early on and had been followed up for four years and hence these samples had been stored longer in the biobank compared to those samples used for the discovery and diagnostic validation. Interestingly, the longer storage time significantly reduced the WID-CIN-index (FIG. 3a; p=0.044), making it impossible to apply the same WID-CIN-index cutoffs as chosen in the previous set. Nevertheless, the WID-CIN-index was elevated in these Cytology-ye samples up to four years prior to the CIN3+ diagnosis (FIG. 3b) with an overall AUC of 0.70 (95% CI: 0.65-0.75) (FIG. 3c). As in the diagnostic set, the performance was better for women >30 years old (AUC 0.78; 95% CI 0.72-0.84) compared to women ≤30 years (AUC 0.63; 95% CI 0.55-0.7) (FIG. 3c, 8b).

### Comparison of Performance of Island and Open-Sea CpGs

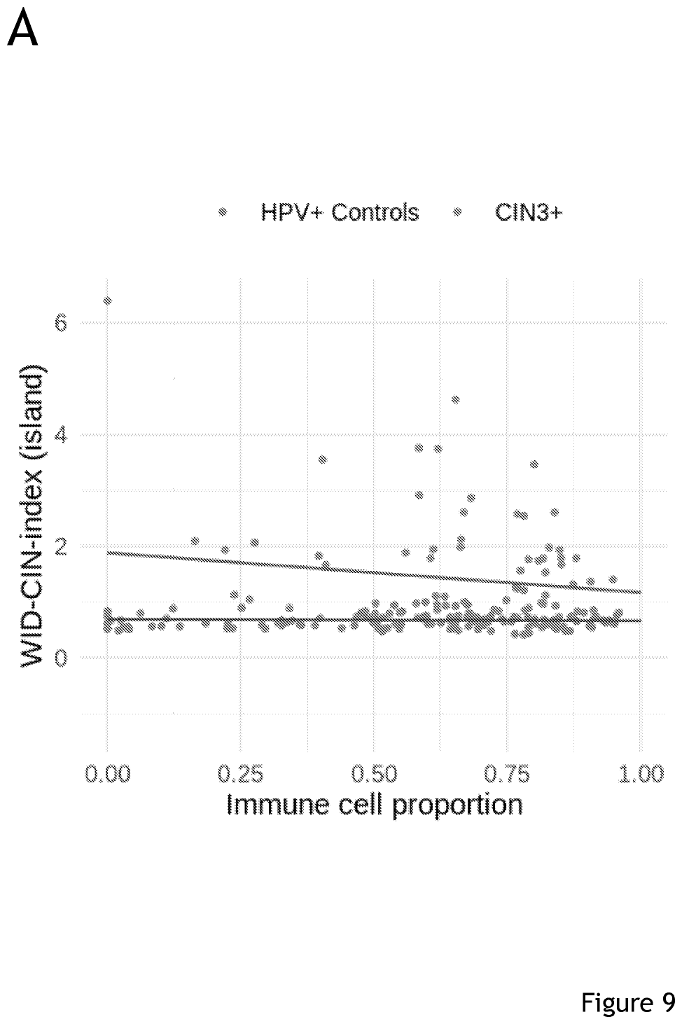

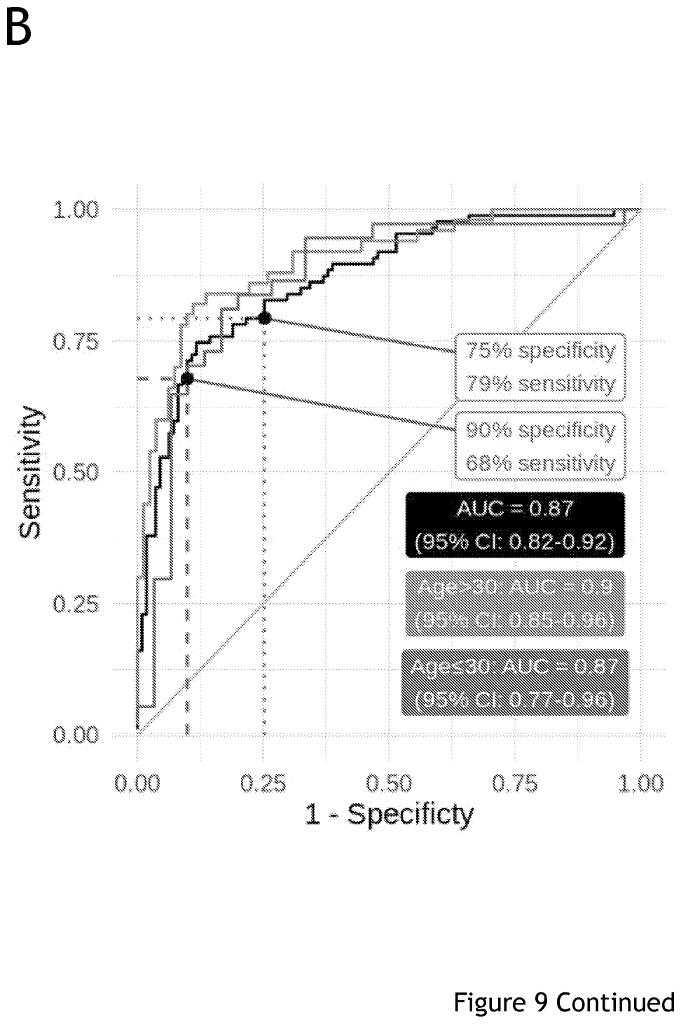

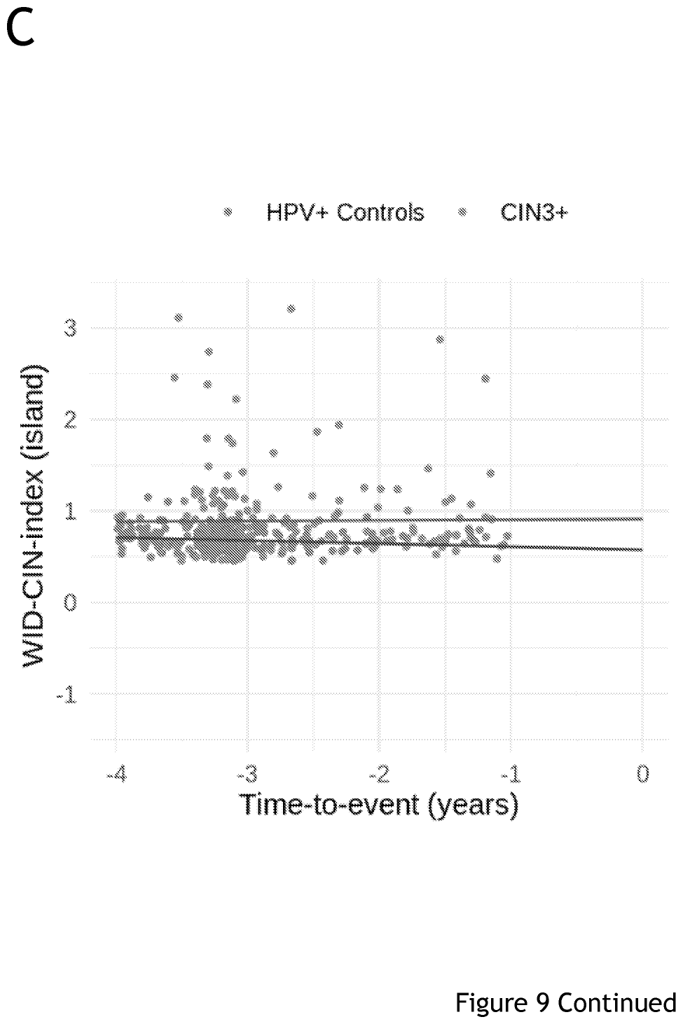

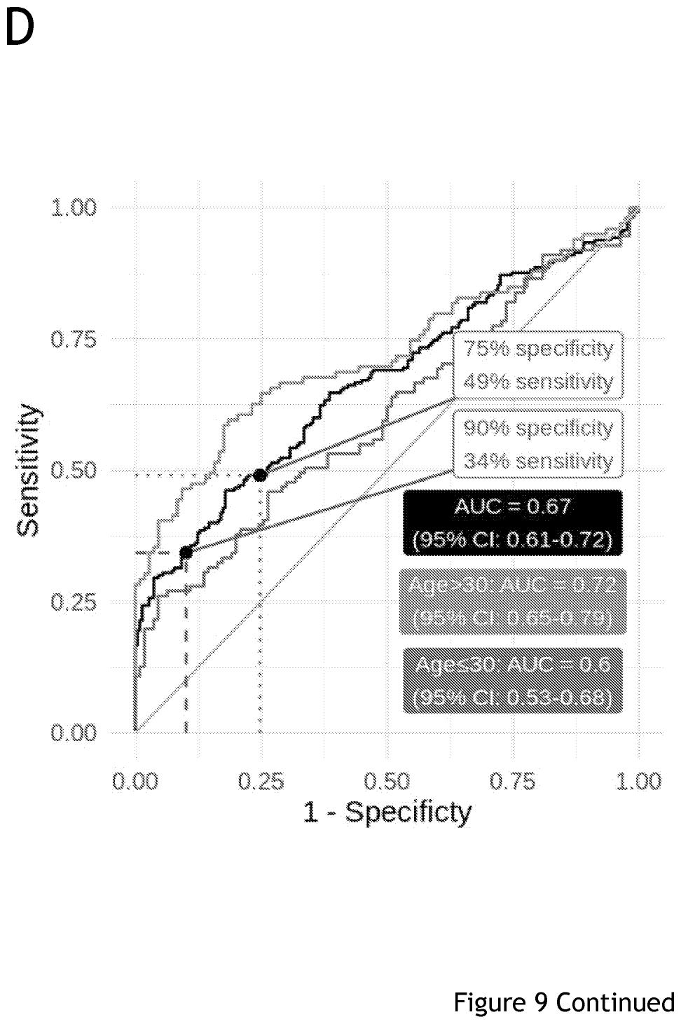

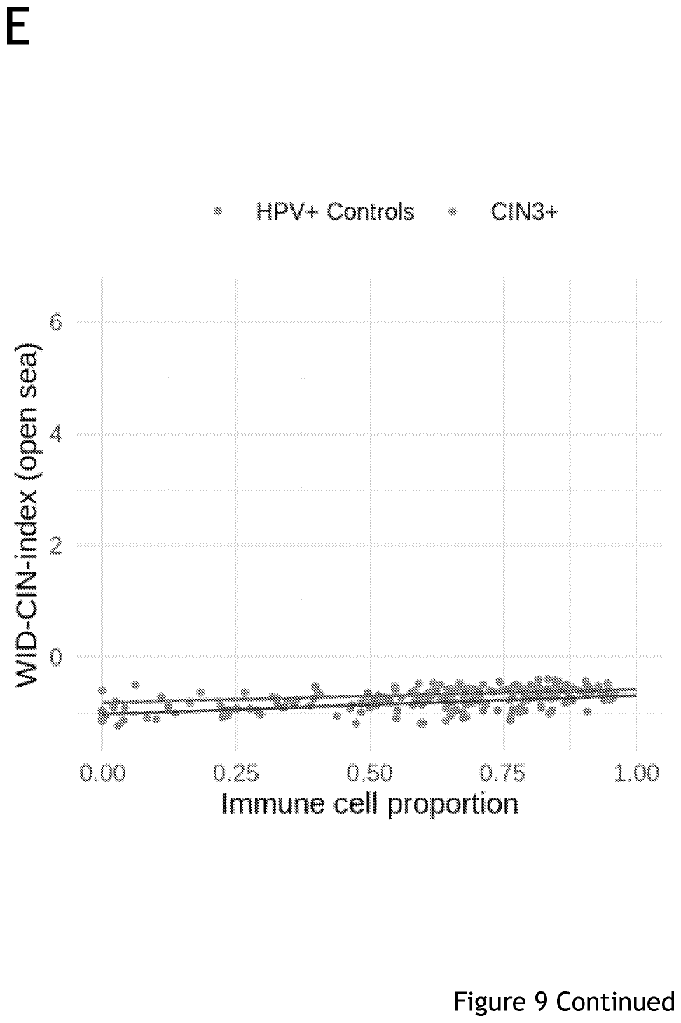

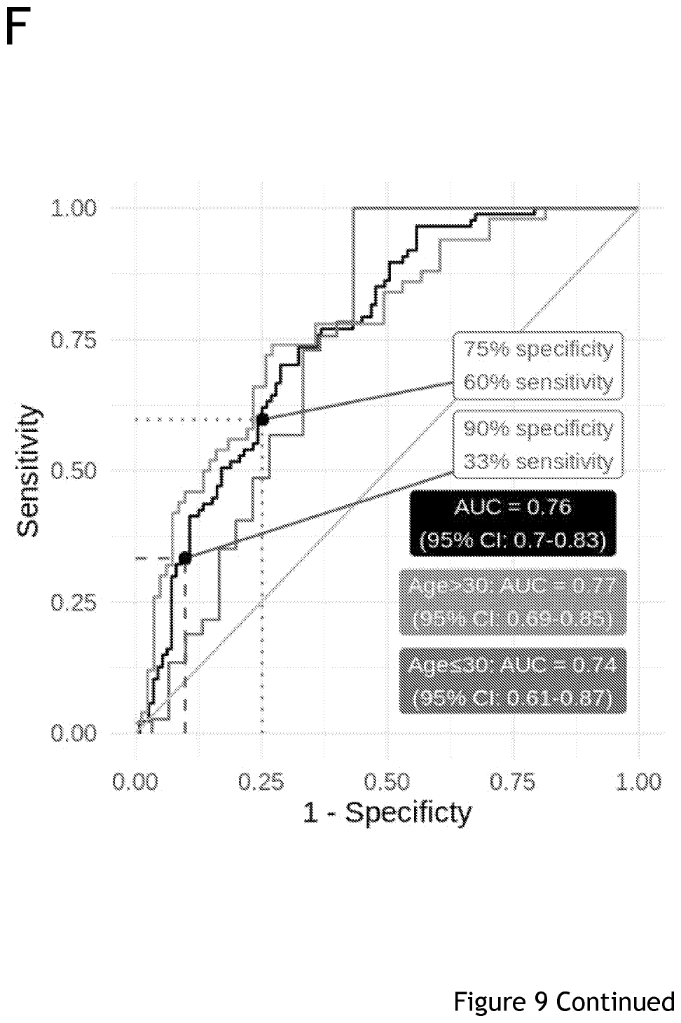

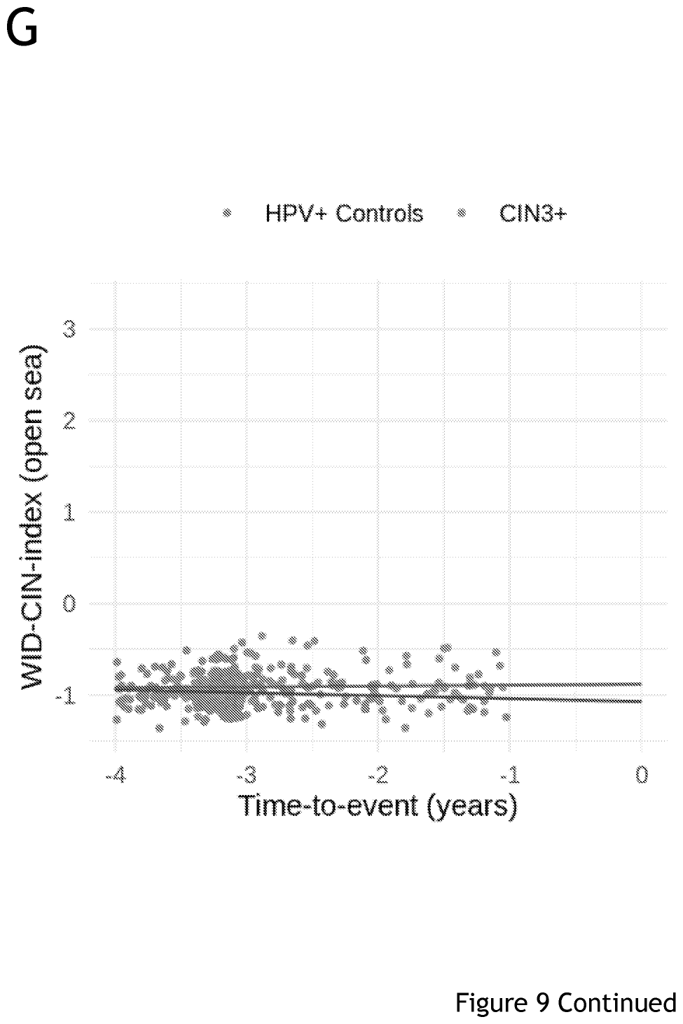

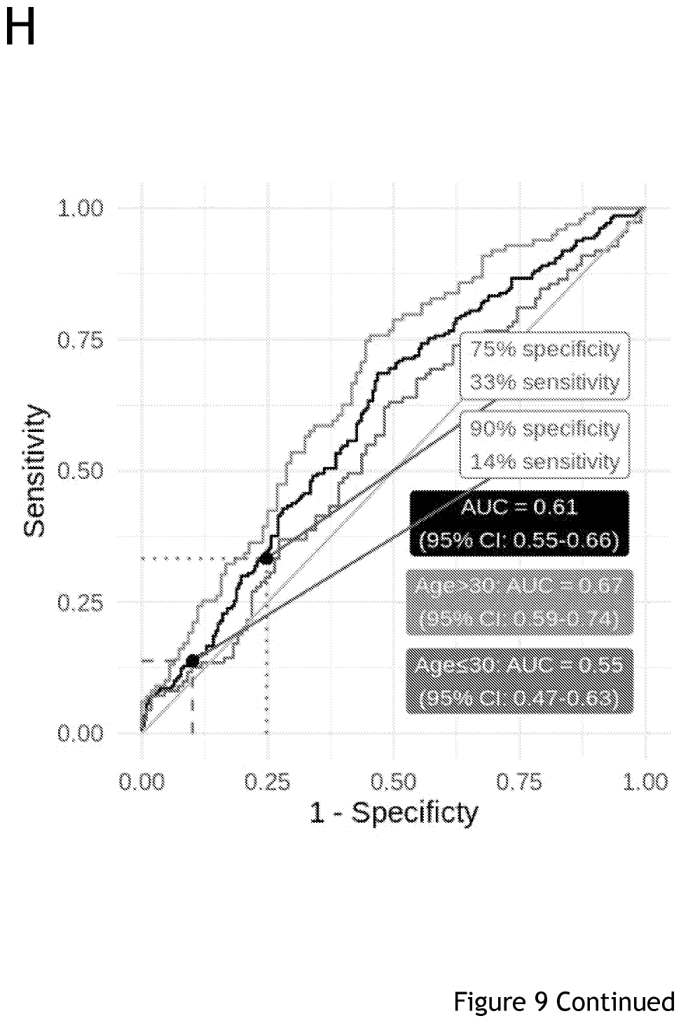

Although the number of island CpGs was under-represented (i.e. only 702 of the 5,000 CpGs were island CpGs), these CpGs carried the highest weight in the WID-CIN-index. Hence, the inventors decomposed the WID-CIN-index into a subcomponent based only on the 702 CpG islands and a subcomponent based on the 3,411 open sea CpGs. The island subcomponent provided an extremely strong signal that corresponded to an AUC of 0.87 in the diagnostic validation set (FIG. 9a,b). It also validated robustly in the predictive validation set with an AUC of 0.67 (FIG. 9c,d). In contrast, the open sea subcomponent consisted of a comparatively weaker signal (AUC of 0.76 in the diagnostic validation set; FIG. 9e,f) which was almost completely lost in the predictive validation set, presumably due to the storage related degradation (FIG. 9g,h).

### Capacity to Detect Endometrial Cancer

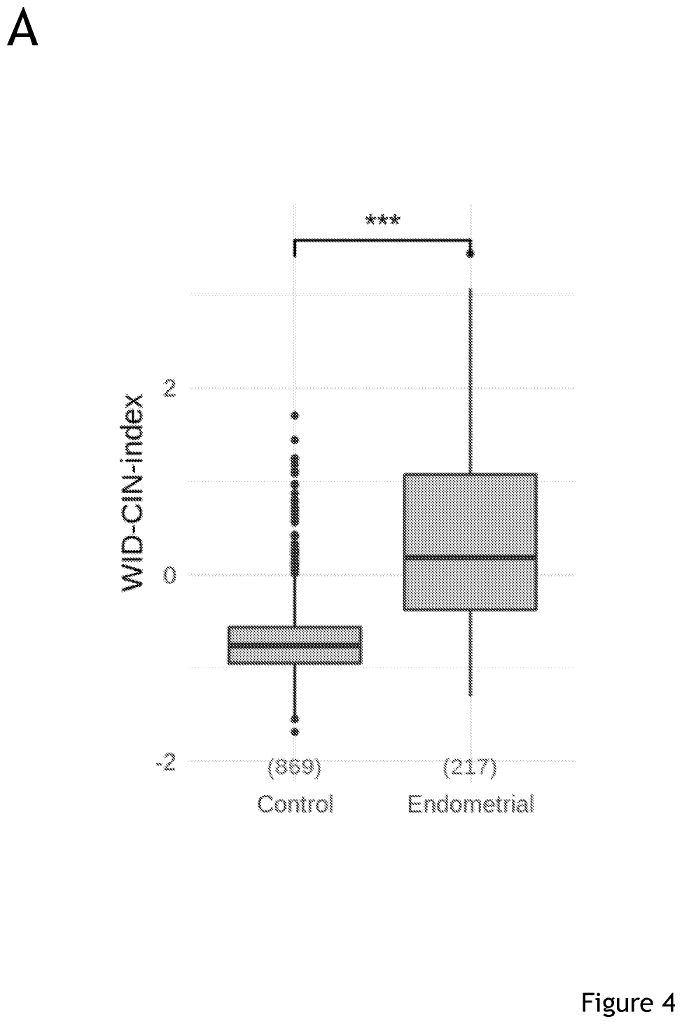

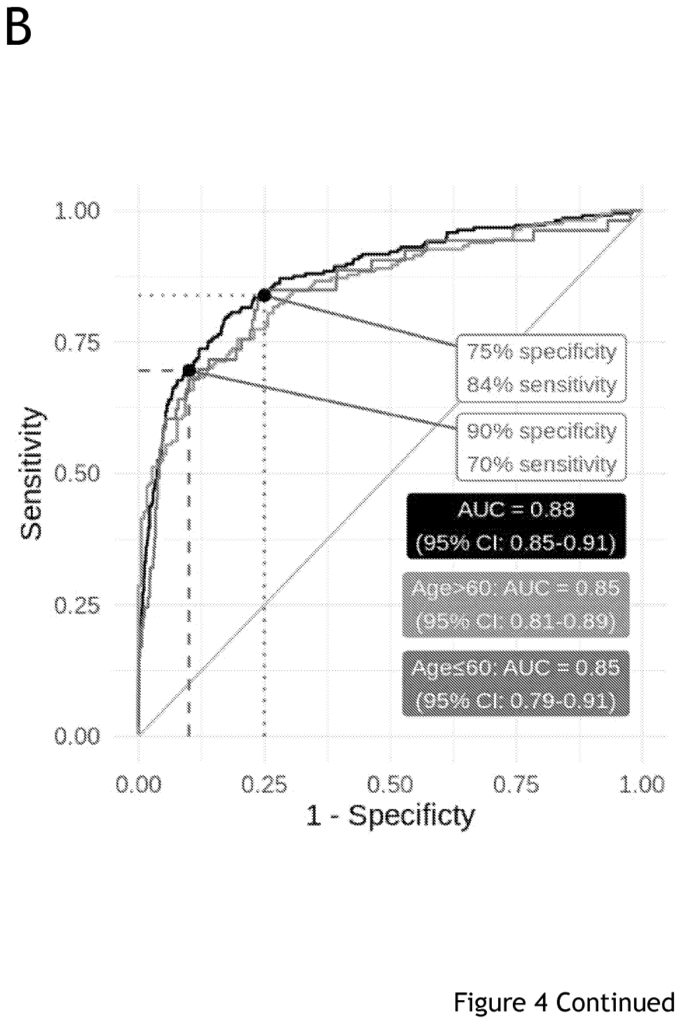

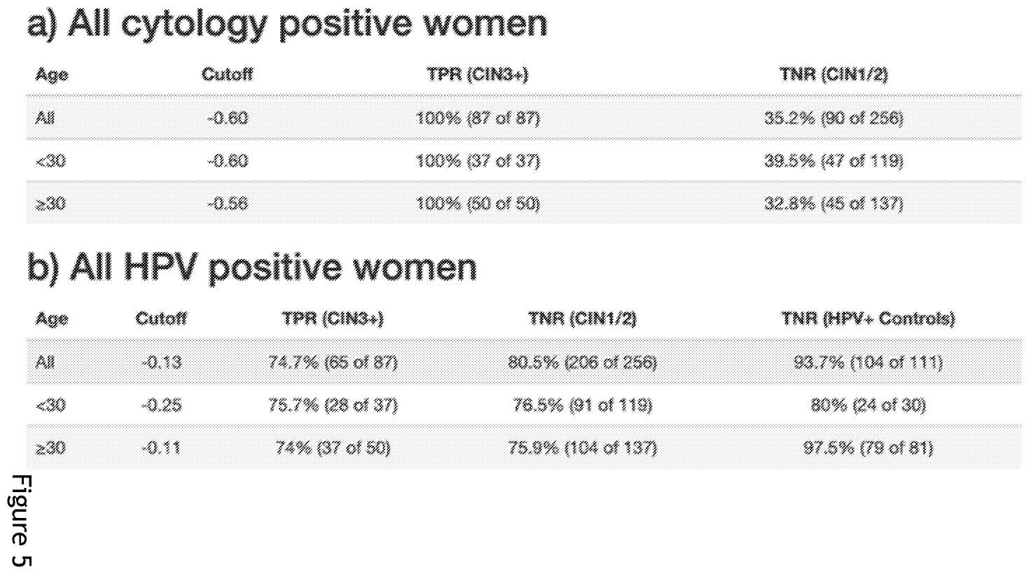

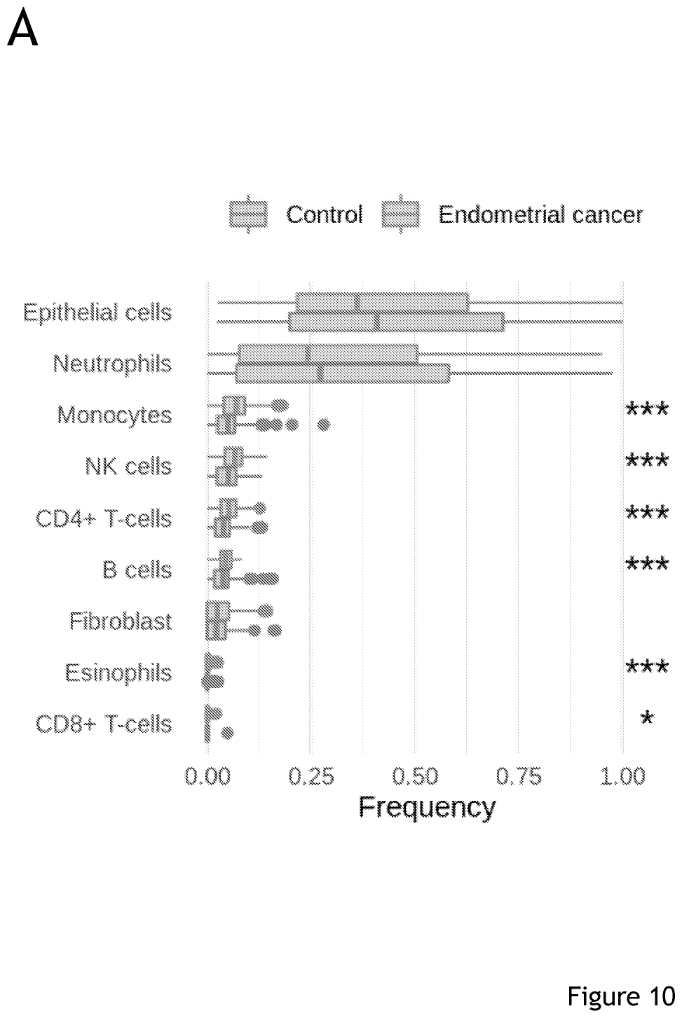

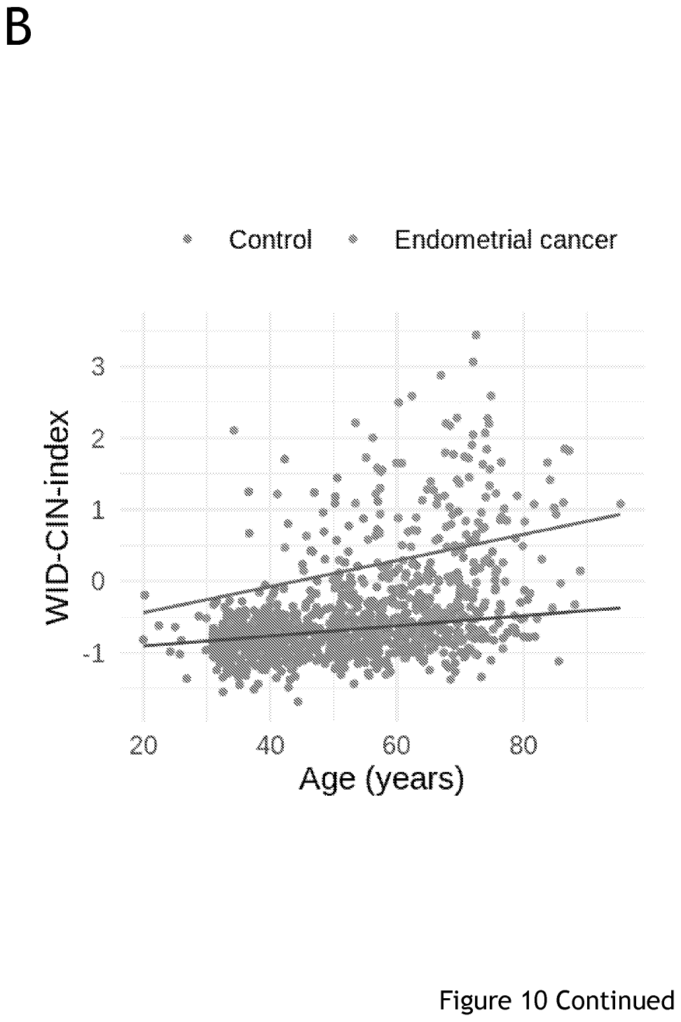

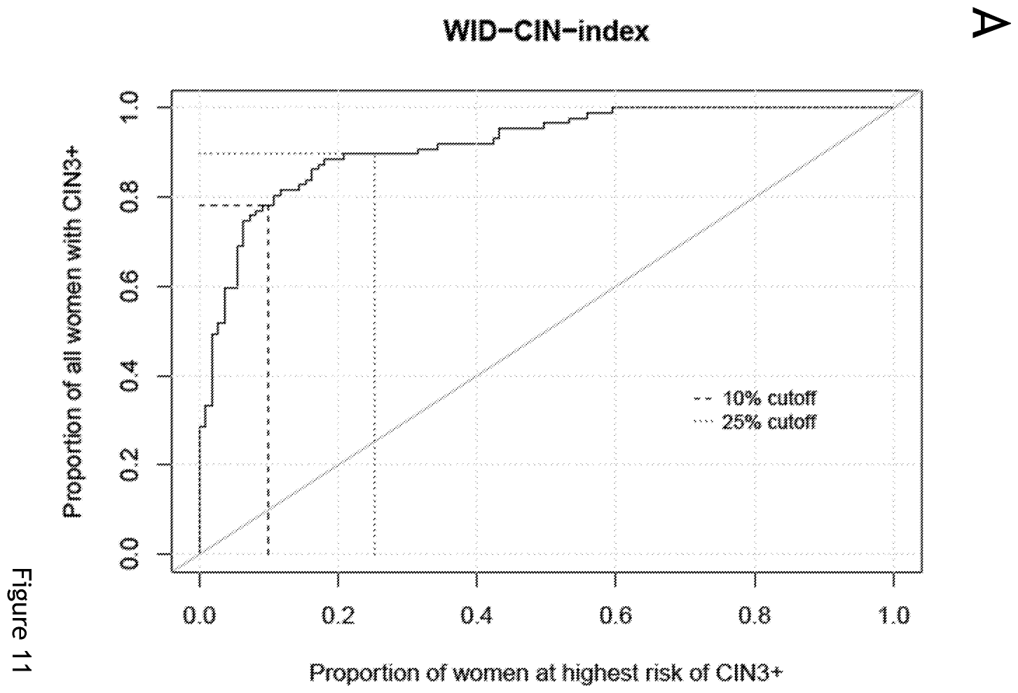

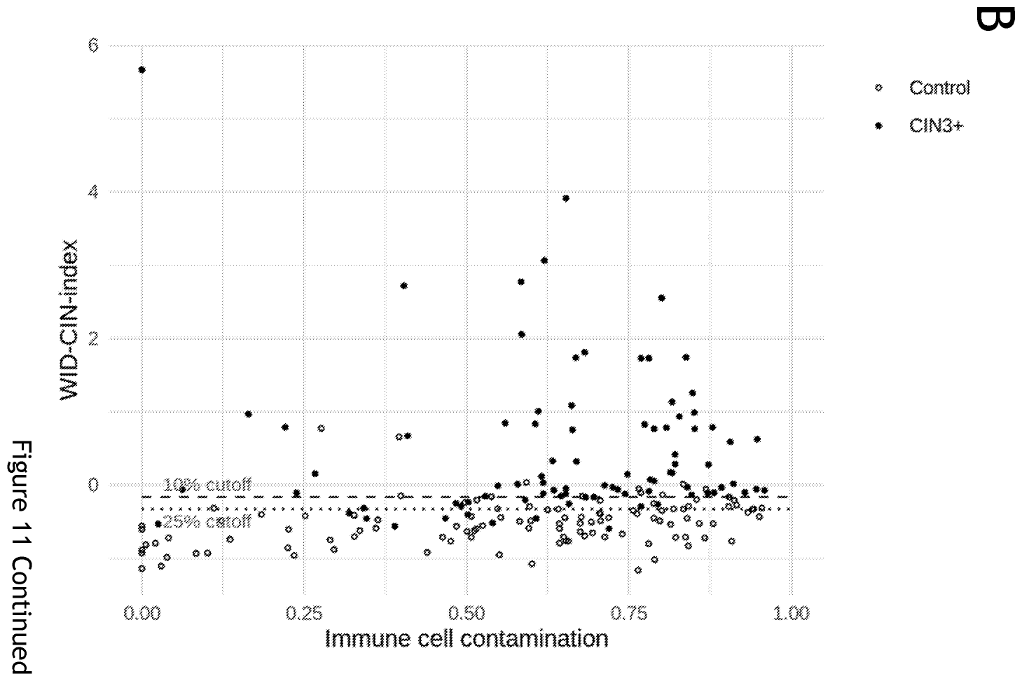

Both the cervical and endometrial epithelium form part of the Müllerian Duct system. Hence, in order to assess whether a woman with a WID-CIN-index positive result, who on colposcopic assessment has no abnormality on her cervix, has an underlying endometrial cancer, the inventors assessed the WID-CIN-index in cervical cytology samples from 217 women with endometrial cancer and 869 healthy controls. The endometrial cancers had a cell-type composition that was broadly similar to the Discovery Set (FIG. 10a). The inventors found that the WID-CIN-test can also identify women with endometrial cancer with a very high accuracy (AUC 0.88; 95% CI: 0.85-0.91). The test was largely independent of age (FIG. 10b) and had an AUC of 0.85 (95% CI 0.81-0.89) for women ≤60 years of age and an AUC of 0.85 (95% CI 0.79-0.91) for older women (FIG. 4b).

### Cancer Index Values and Clinical Actions

Four sub-groups defined by ranges of cancer index values are specified in Table 9 as corresponding to preferred clinical actions, comprising intensified screening, administration of therapeutics and surgery. The subgroups are based on control samples from the internal validation set. That is, these values of the index split the control samples into four equally sized groups. Odds ratio values are calculated by comparing the number of cases and controls in a given quartile to the first quartile (which is taken as a reference). Odds ratio values are determined for CIN3 risk and endometrial cancer risk. For example, a woman in the fourth quartile is roughly 104 times more likely to have CIN3 than a woman in the first quartile, and approximately 40 times more likely to have endometrial cancer than a woman in the first quartile.

### Discussion

In general, cervical cancer screening is one of the foremost success stories in medicine and oncology in particular. Here the inventors have provided extensive evidence that an objective DNA methylation signature, the WID-CIN-test, outperforms cytology as a tool to triage HPV+ve women. The inventors have demonstrated that the WID-CIN test (i) reduces the number of false positive HPV+ women by 50%, (ii) does not identify women with CIN1 and only half of women with CIN2 (i.e. likely those which progress to CIN3+ if not treated before), (iii) is able—despite the cytology being negative—to identify HPV+ve women which present with a CIN3+ up to four years later, and (iv) is able to also identify the majority of women with endometrial cancer.

Whereas a plethora of DNA methylation markers have been identified and assessed in cervical liquid-based cytology samples and deemed to be promising only a small number of studies assessed the clinical validity of these markers in a screening setting. Using DNA methylation levels of a combination of two genes (i.e. MAL and miR-124-2), Verhoef et al demonstrated in a prospective clinical trial that triaging HPV+ve women with DNA methylation somehow lowers sensitivity (67.5%) compared to cytology-triaging (74.8%) and required almost twice as many colposcopy referrals. As this study was performed on women aged 33 years or older, the performance of these methylation markers would have been worse in younger women. Although the inventors also observed this age-dependent performance in the WID-test, in young women the sensitivity was 80% at a 75% specificity.

To date, the considerable heterogeneity of cervical liquid-based cytology samples has been entirely underappreciated (specifically at the level of DNA which includes DNA from cell-debris not visible at the microscopic level when assessing cytology), their high variability of epithelial and immune cell proportion ranging from almost no immune cells to samples which almost exclusively consist of immune cells. The inventors have thoroughly assessed and concluded that the WID-test performance is independent of sample heterogeneity and hence, likely to perform equally well in self-collected samples.

Our observation that the WID-test is able to identify HPV+ve women who show no abnormal cells in their cervical liquid-based cytology sample but develop CIN3+ up to four years later might suggest that the WID-test is not only reflective of an epigenetic cancer program, but may in fact be reflective of an individual predisposition to progress to a cervical (pre-)cancer upon infection with HPV. In order to test this hypothesis, samples from women prior to HPV-infection will need to be analysed in order to assess whether the WID-test would have predicted the disease development even before the presence of the carcinogen.

Here the inventors have demonstrated the unprecedented performance of a DNA methylation classifier—the WID-CIN-test—in identifying HPV+ve women with or at risk of CIN3+. The fact that the test not only identifies women with CIN3+(and equally as well when making comparisons to HPV+ve or HPV-ve women) but also women with endometrial cancer strongly suggests that the WID-CIN-test be rapidly introduced and implemented in the clinical arena.

TABLE 9

| | WID- | CIN3 or | | |
|---|---|---|---|---|
| | CIN- | invasive | | |
| | index | cervical cancer | Endometrial | |
| | range | risk | cancer risk | Clinical Action |
| WID-CIN index (cutpoint for | (−1.17, | 1.00 | 1.00 | no clinical action, repeat of cervical smear in 1 year for HPV positive |
| <50% specificity) | −0.53) | (reference) | (reference) | women and in 3 years for HPV negative women |
| WID-CIN index (cutpoint for | (−0.53, | 3.99 | 4.7 | no immediate clinical action, repeat of cervical smear in 1 year |
| 50%-75% specificity) | −0.33) | (0.94, 21.29) | (2.65,8.2) | |
| WID-CIN index (cutpoint for | (−0.33, | 10.29 | 5.89 | Immediate colposcopy for HPV positive women and repeat cervical smear |
| 75%-90% specificity) | −0.17) | (2.74, 52.77) | (2.73, 12.04) | in 1 year for HPV negative women; Asessment of endometrium in case |
| | | | | colposcopy is negative |
| WID-CIN index (cutpoint for | (−0.17, | 103.97 | 40.44 | Immediate colposcopy irrespective of HPV-status; Immediate endometrial |
| >90 specificity) | 0.77) | (31.61, 498.89) | (26.22, 64) | biopsy and hysteroscopy if colposcopy is negative |
| CLEC14A_i | | PMR >5 | PMR >5 | Immediate colposcopy; if colpsocopy negative then endometrial biopsy; if |
| | | | | both negative repeat test in 1 year |
| CLVS2_ii | | PMR >1 | PMR >1 | Immediate colposcopy; if colpsocopy negative then endometrial biopsy; if |
| | | | | both negative repeat test in 1 year |
| DPP6_i | | PMR >2 | PMR >2 | Immediate colposcopy; if colpsocopy negative then endometrial biopsy; if |
| | | | | both negative repeat test in 1 year |
| ELAVL4_i | | PMR >5 | PMR >5 | Immediate colposcopy; if colpsocopy negative then endometrial biopsy; if |
| | | | | both negative repeat test in 1 year |
| FUT9_ii | | PMR >4 | PMR >4 | Immediate colposcopy; if colpsocopy negative then endometrial biopsy; if |
| | | | | both negative repeat test in 1 year |
| GALR1_i 9 | | PMR >4 | PMR >4 | Immediate colposcopy; if colpsocopy negative then endometrial biopsy; if |
| | | | | both negative repeat test in 1 year |
| GSX1_iii | | PMR >1 | PMR >1 | Immediate colposcopy; if colpsocopy negative then endometrial biopsy; if |
| | | | | both negative repeat test in 1 year |
| KCNIP4_i | | PMR >1 | PMR >1 | Immediate colposcopy; if colpsocopy negative then endometrial biopsy; if |
| | | | | both negative repeat test in 1 year |
| LHX8_i | | PMR >4 | PMR >4 | Immediate colposcopy; if colpsocopy negative then endometrial biopsy; if |
| | | | | both negative repeat test in 1 year |
| LINC00403_i | | PMR >4 | PMR >4 | Immediate colposcopy; if colpsocopy negative then endometrial biopsy; if |
| | | | | both negative repeat test in 1 year |
| LINC00403_iv | | PMR >4 | PMR >4 | Immediate colposcopy; if colpsocopy negative then endometrial biopsy; if |
| | | | | both negative repeat test in 1 year |
| MDGA_I | | PMR >5 | PMR >5 | Immediate colposcopy; if colpsocopy negative then endometrial biopsy; if |
| | | | | both negative repeat test in 1 year |
| MHC_ii | | PMR >2 | PMR >2 | Immediate colposcopy; if colpsocopy negative then endometrial biopsy; if |
| | | | | both negative repeat test in 1 year |
| MSC_ii | | PMR >1 | PMR >1 | Immediate colposcopy; if colpsocopy negative then endometrial biopsy; if |
| | | | | both negative repeat test in 1 year |
| NET01_i | | PMR >5 | PMR >5 | Immediate colposcopy; if colpsocopy negative then endometrial biopsy; if |
| | | | | both negative repeat test in 1 year |
| NID2_i | | PMR >5 | PMR >5 | Immediate colposcopy; if colpsocopy negative then endometrial biopsy; if |
| | | | | both negative repeat test in 1 year |
| NTM_ii | | PMR >0 | PMR >0 | Immediate colposcopy; if colpsocopy negative then endometrial biopsy; if |
| | | | | both negative repeat test in 1 year |
| PDEIC_ii | | PMR >4 | PMR >4 | Immediate colposcopy; if colpsocopy negative then endometrial biopsy; if |
| | | | | both negative repeat test in 1 year |
| RALYL_ii | | PMR >2 | PMR >2 | Immediate colposcopy; if colpsocopy negative then endometrial biopsy; if |
| | | | | both negative repeat test in 1 year |
| RBFOX_i | | PMR >7 | PMR >7 | Immediate colposcopy; if colpsocopy negative then endometrial biopsy; if |
| | | | | both negative repeat test in 1 year |
| RCN1_i | | PMR >6 | PMR >6 | Immediate colposcopy; if colpsocopy negative then endometrial biopsy; if |
| | | | | both negative repeat test in 1 year |
| REG39_i | | PMR >5 | PMR >5 | Immediate colposcopy; if colpsocopy negative then endometrial biopsy; if |
| | | | | both negative repeat test in 1 year |
| RNF291_ii | | PMR >6 | PMR >6 | Immediate colposcopy; if colpsocopy negative then endometrial biopsy; if |
| | | | | both negative repeat test in 1 year |
| RP4_ii | | PMR >6 | PMR >6 | Immediate colposcopy; if colpsocopy negative then endometrial biopsy; if |
| | | | | both negative repeat test in 1 year |
| RYR2_i | | PMR >5 | PMR >5 | Immediate colposcopy; if colpsocopy negative then endometrial biopsy; if |
| | | | | both negative repeat test in 1 year |
| SERTM1_ii | | PMR >2 | PMR >2 | Immediate colposcopy; if colpsocopy negative then endometrial biopsy; if |
| | | | | both negative repeat test in 1 year |
| ZIC4 | | PMR >7 | PMR >7 | Immediate colposcopy; if colpsocopy negative then endometrial biopsy; if |
| | | | | both negative repeat test in 1 year |
| ZNF536_ii | | PMR >3 | PMR >3 | Immediate colposcopy; if colpsocopy negative then endometrial biopsy; if |
| | | | | both negative repeat test in 1 year |

TABLE 10

| | | | | | | | | | | | | | Amp- | | | | | | |
|---|---|---|---|---|---|---|---|---|---|---|---|---|---|---|---|---|---|---|---|
| | | | | | | | | | | | | | licon | | | | | | |
| | | | | Spec- | Sen- | | | | | | | | se- | | | | | | |
| | | | | i- | si- | ML | | Chro- | | | | Amp- | quence | F | F | R | R | | |
| Rxn | Gene | | Cut- | fic- | tiv- | design | DNA | mo- | Rxn | Rxn | Amplicon | licon. | 5′ to | Primer | Primer | Primer | Primer | Probe. | Probe. |
| name | name | auc | off | ity | ity | region | strand | some | Start | Finish | Location | Length | 3′ | Start | Finish | Start | Finish | Start | Finish |
| DPP6_i | DPP6 | 0.95 | 0 | 0.75 | 0.86 | chr7: | posi- | 7 | 1535840 | 1535840 | chr7: | 72 | TCAC | 1535840 | 1535840 | 1535840 | 1535840 | 1535840 | 1535840 |
| | | | | | | 153583701- | tive | | 08 | 79 | 153584008- | | [[CG]] | 08 | 34 | 60 | 79 | 42 | 58 |
| | | | | | | 153584375 | | | | | 153584079 | | TAGTGC | | | | | | |
| | | | | | | | | | | | | | TTGTTT | | | | | | |
| | | | | | | | | | | | | | GTGGAA | | | | | | |
| | | | | | | | | | | | | | GC[CG] | | | | | | |
| | | | | | | | | | | | | | AG[CG] | | | | | | |
| | | | | | | | | | | | | | TG | | | | | | |
| | | | | | | | | | | | | | [[CG]] | | | | | | |
| | | | | | | | | | | | | | TG | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | [[CG]] | | | | | | |
| | | | | | | | | | | | | | [[CG]] | | | | | | |
| | | | | | | | | | | | | | CACCCA | | | | | | |
| | | | | | | | | | | | | | GTCCAG | | | | | | |
| | | | | | | | | | | | | | [[CG]] | | | | | | |
| | | | | | | | | | | | | | [[CG]] | | | | | | |
| | | | | | | | | | | | | | GAGTGG | | | | | | |
| | | | | | | | | | | | | | G | | | | | | |
| CLEC14A_i | CLEC14A | 0.92 | 3.82 | 0.75 | 0.9 | chr14: | posi- | 14 | 387245 | 387246 | chr14: | 83 | T | 387245 | 387246 | 387246 | 387246 | 387246 | 387246 |
| | | | | | | 38724342- | tive | | 96 | 78 | 38724596- | | [[CG]] | 96 | 20 | 58 | 78 | 32 | 55 |
| | | | | | | 38725008 | | | | | 38724678 | | GTCCCA | | | | | | |
| | | | | | | | | | | | | | GGTGGA | | | | | | |
| | | | | | | | | | | | | | CTGAAG | | | | | | |
| | | | | | | | | | | | | | TCCAGA | | | | | | |
| | | | | | | | | | | | | | G[CG]G | | | | | | |
| | | | | | | | | | | | | | [CG]CT | | | | | | |
| | | | | | | | | | | | | | GTGCAG | | | | | | |
| | | | | | | | | | | | | | CTGGAA | | | | | | |
| | | | | | | | | | | | | | GGG | | | | | | |
| | | | | | | | | | | | | | [[CG]] | | | | | | |
| | | | | | | | | | | | | | [[CG]] | | | | | | |
| | | | | | | | | | | | | | [[CG]] | | | | | | |
| | | | | | | | | | | | | | ATAGCT | | | | | | |
| | | | | | | | | | | | | | CAAGTT | | | | | | |
| | | | | | | | | | | | | | AGAGG | | | | | | |
| | | | | | | | | | | | | | [[CG]] | | | | | | |
| | | | | | | | | | | | | | GCCC | | | | | | |
| | | | | | | | | | | | | | [[CG]] | | | | | | |
| | | | | | | | | | | | | | GG | | | | | | |
| RALYL_ii | RALYL | 0.93 | 0.03 | 0.75 | 0.88 | chr8: | nega- | 8 | 850954 | 850955 | chr8: | 89 | CCTGCT | 850955 | 850955 | 850954 | 850955 | 850955 | 850955 |
| | | | | | | 85095225- | tive | | 92 | 80 | 85095492- | | [[CG]] | 56 | 80 | 92 | 17 | 20 | 41 |
| | | | | | | 85095824 | | | | | 85095580 | | TCTGGG | | | | | | |
| | | | | | | | | | | | | | CTCACA | | | | | | |
| | | | | | | | | | | | | | G | | | | | | |
| | | | | | | | | | | | | | [[CG]] | | | | | | |
| | | | | | | | | | | | | | AAGGCA | | | | | | |
| | | | | | | | | | | | | | GCCT | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | AGCTGC | | | | | | |
| | | | | | | | | | | | | | [[CG]] | | | | | | |
| | | | | | | | | | | | | | CTGC | | | | | | |
| | | | | | | | | | | | | | [CG]CT | | | | | | |
| | | | | | | | | | | | | | GCTGC | | | | | | |
| | | | | | | | | | | | | | [CG]CC | | | | | | |
| | | | | | | | | | | | | | ACTGGT | | | | | | |
| | | | | | | | | | | | | | GTTGC | | | | | | |
| | | | | | | | | | | | | | [[CG]] | | | | | | |
| | | | | | | | | | | | | | CTCTCA | | | | | | |
| | | | | | | | | | | | | | GG | | | | | | |
| | | | | | | | | | | | | | [[CG]] | | | | | | |
| | | | | | | | | | | | | | C | | | | | | |
| MSC_ii | MSC | 0.91 | 0.47 | 0.75 | 0.85 | chr8: | nega- | 8 | 727562 | 727563 | chr8: | 82 | CTCCTC | 727563 | 727563 | 727562 | 727562 | 727562 | 727563 |
| | | | | | | 72755932- | tive | | 67 | 48 | 72756267- | | TGC | 24 | 48 | 67 | 92 | 97 | 22 |
| | | | | | | 72756681 | | | | | 72756348 | | [[CG]] | | | | | | |
| | | | | | | | | | | | | | A | | | | | | |
| | | | | | | | | | | | | | [[CG]] | | | | | | |
| | | | | | | | | | | | | | AGTTGT | | | | | | |
| | | | | | | | | | | | | | CACTGG | | | | | | |
| | | | | | | | | | | | | | G[CG]A | | | | | | |
| | | | | | | | | | | | | | GG | | | | | | |
| | | | | | | | | | | | | | [[CG]] | | | | | | |
| | | | | | | | | | | | | | TAGCTG | | | | | | |
| | | | | | | | | | | | | | [[CG]] | | | | | | |
| | | | | | | | | | | | | | CTCTA | | | | | | |
| | | | | | | | | | | | | | [[CG]] | | | | | | |
| | | | | | | | | | | | | | [[CG]] | | | | | | |
| | | | | | | | | | | | | | [[CG]] | | | | | | |
| | | | | | | | | | | | | | GAGGGG | | | | | | |
| | | | | | | | | | | | | | [[CG]] | | | | | | |
| | | | | | | | | | | | | | GCCTCT | | | | | | |
| | | | | | | | | | | | | | TGGAGG | | | | | | |
| | | | | | | | | | | | | | [[CG]] | | | | | | |
| | | | | | | | | | | | | | GGGACC | | | | | | |
| GSX1_iii | GSX1 | 0.93 | 0.02 | 0.78 | 0.75 | chr13: | posi- | 13 | 283667 | 283668 | chr13: | 82 | [[CG]] | 283667 | 283668 | 283668 | 283668 | 283668 | 283668 |
| | | | | | | 28366477- | tive | | 85 | 66 | 28366785- | | CAGAGG | 85 | 02 | 47 | 66 | 10 | 23 |
| | | | | | | 28367151 | | | | | 28366866 | | G | | | | | | |
| | | | | | | | | | | | | | [[CG]] | | | | | | |
| | | | | | | | | | | | | | GGCTGG | | | | | | |
| | | | | | | | | | | | | | CTG | | | | | | |
| | | | | | | | | | | | | | [CG]GG | | | | | | |
| | | | | | | | | | | | | | G | | | | | | |
| | | | | | | | | | | | | | [[CG]] | | | | | | |
| | | | | | | | | | | | | | AC | | | | | | |
| | | | | | | | | | | | | | [[CG]] | | | | | | |
| | | | | | | | | | | | | | [[CG]] | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | GGGCCA | | | | | | |
| | | | | | | | | | | | | | TGC | | | | | | |
| | | | | | | | | | | | | | [CG] | | | | | | |
| | | | | | | | | | | | | | [CG]CT | | | | | | |
| | | | | | | | | | | | | | CCTTCC | | | | | | |
| | | | | | | | | | | | | | TGGTGG | | | | | | |
| | | | | | | | | | | | | | ACT | | | | | | |
| | | | | | | | | | | | | | [[CG]] | | | | | | |
| | | | | | | | | | | | | | CTAGTG | | | | | | |
| | | | | | | | | | | | | | CTG | | | | | | |
| | | | | | | | | | | | | | [[CG]] | | | | | | |
| | | | | | | | | | | | | | C | | | | | | |
| ZNF536_ii | ZNF536 | 0.92 | 1.73 | 0.75 | 0.75 | chr19: | nega- | 19 | 308661 | 308662 | chr19: | 85 | GAG | 308662 | 308662 | 308661 | 308662 | 308662 | 308662 |
| | | | | | | 30865857- | tive | | 95 | 79 | 30866195- | | [[CG]] | 61 | 79 | 95 | 14 | 35 | 58 |
| | | | | | | 30866531 | | | | | 30866279 | | GC | | | | | | |
| | | | | | | | | | | | | | [[CG]] | | | | | | |
| | | | | | | | | | | | | | CACTTC | | | | | | |
| | | | | | | | | | | | | | ACCTTA | | | | | | |
| | | | | | | | | | | | | | [CG]GA | | | | | | |
| | | | | | | | | | | | | | GGGGAG | | | | | | |
| | | | | | | | | | | | | | ATAATG | | | | | | |
| | | | | | | | | | | | | | AGATCA | | | | | | |
| | | | | | | | | | | | | | ATTAGA | | | | | | |
| | | | | | | | | | | | | | GG | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | TCAC | | | | | | |
| | | | | | | | | | | | | | [[CG]] | | | | | | |
| | | | | | | | | | | | | | [[CG]] | | | | | | |
| | | | | | | | | | | | | | C[CG]G | | | | | | |
| | | | | | | | | | | | | | AGACAG | | | | | | |
| | | | | | | | | | | | | | CTGC | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | CA | | | | | | |
| CLVS2_ii | CLVS2 | 0.93 | 0.18 | 0.8 | 0.75 | chr6: | nega- | 6 | 1233174 | 1233175 | chr6: | 95 | [[CG]] | 1233175 | 1233175 | 1233174 | 1233174 | 1233174 | 1233174 |
| | | | | | | 123317188- | tive | | 40 | 34 | 123317440- | | GAC | 10 | 34 | 40 | 58 | 70 | 88 |
| | | | | | | 123317787 | | | | | 123317534 | | [[CG]] | | | | | | |
| | | | | | | | | | | | | | CT | | | | | | |
| | | | | | | | | | | | | | [[CG]] | | | | | | |
| | | | | | | | | | | | | | GGAGAG | | | | | | |
| | | | | | | | | | | | | | CCCCAG | | | | | | |
| | | | | | | | | | | | | | GAGAGG | | | | | | |
| | | | | | | | | | | | | | CCAG | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | CAGCAG | | | | | | |
| | | | | | | | | | | | | | C | | | | | | |
| | | | | | | | | | | | | | [[CG]] | | | | | | |
| | | | | | | | | | | | | | CCC | | | | | | |
| | | | | | | | | | | | | | [CG]CT | | | | | | |
| | | | | | | | | | | | | | G[CG]C | | | | | | |
| | | | | | | | | | | | | | CCACCT | | | | | | |
| | | | | | | | | | | | | | CCC | | | | | | |
| | | | | | | | | | | | | | [CG]GC | | | | | | |
| | | | | | | | | | | | | | TGCTCC | | | | | | |
| | | | | | | | | | | | | | [[CG]] | | | | | | |
| | | | | | | | | | | | | | GAGGGC | | | | | | |
| | | | | | | | | | | | | | TCACAA | | | | | | |
| | | | | | | | | | | | | | AG | | | | | | |
| KCNIP4_i | KCNIP4 | 0.93 | 0.72 | 0.75 | 0.9 | chr4: | posi- | 4 | 219505 | 219505 | chr4: | 62 | GAAG | 219505 | 219505 | 219505 | 219505 | 219505 | 219505 |
| | | | | | | 21950268- | tive | | 35 | 96 | 21950535- | | [[CG]] | 35 | 50 | 72 | 96 | 52 | 69 |
| | | | | | | 21950867 | | | | | 21950596 | | GG | | | | | | |
| | | | | | | | | | | | | | [[CG]] | | | | | | |
| | | | | | | | | | | | | | GGCTGC | | | | | | |
| | | | | | | | | | | | | | A | | | | | | |
| | | | | | | | | | | | | | [[CG]] | | | | | | |
| | | | | | | | | | | | | | GG | | | | | | |
| | | | | | | | | | | | | | [[CG]] | | | | | | |
| | | | | | | | | | | | | | GCT | | | | | | |
| | | | | | | | | | | | | | [[CG]] | | | | | | |
| | | | | | | | | | | | | | AGTG | | | | | | |
| | | | | | | | | | | | | | [[CG]] | | | | | | |
| | | | | | | | | | | | | | GGGACC | | | | | | |
| | | | | | | | | | | | | | CCAGCC | | | | | | |
| | | | | | | | | | | | | | CCT | | | | | | |
| | | | | | | | | | | | | | [[CG]] | | | | | | |
| | | | | | | | | | | | | | CCCT | | | | | | |
| | | | | | | | | | | | | | [[CG]] | | | | | | |
| | | | | | | | | | | | | | TGAGC | | | | | | |
| NTM_ii | NTM | 0.96 | 0.47 | 0.85 | 0.75 | chr11: | nega- | 11 | 1317812 | 1317813 | chr11: | 103 | C | 1317813 | 1317813 | 1317812 | 1317812 | 1317813 | 1317813 |
| | | | | | | 131780961- | tive | | 52 | 54 | 131781252- | | [[CG]] | 31 | 54 | 52 | 66 | 00 | 27 |
| | | | | | | 131781560 | | | | | 131781354 | | GT | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | GGTTCA | | | | | | |
| | | | | | | | | | | | | | C[CG]C | | | | | | |
| | | | | | | | | | | | | | TCAGTC | | | | | | |
| | | | | | | | | | | | | | CC[CG] | | | | | | |
| | | | | | | | | | | | | | [CG]CT | | | | | | |
| | | | | | | | | | | | | | [CG]CT | | | | | | |
| | | | | | | | | | | | | | C[CG]C | | | | | | |
| | | | | | | | | | | | | | ACCCCA | | | | | | |
| | | | | | | | | | | | | | CCCACT | | | | | | |
| | | | | | | | | | | | | | TCCTGT | | | | | | |
| | | | | | | | | | | | | | GCT | | | | | | |
| | | | | | | | | | | | | | [[CG]] | | | | | | |
| | | | | | | | | | | | | | CC | | | | | | |
| | | | | | | | | | | | | | [[CG]] | | | | | | |
| | | | | | | | | | | | | | GGGGG | | | | | | |
| | | | | | | | | | | | | | [CG]TG | | | | | | |
| | | | | | | | | | | | | | TGC | | | | | | |
| | | | | | | | | | | | | | [[CG]] | | | | | | |
| | | | | | | | | | | | | | TG | | | | | | |
| | | | | | | | | | | | | | [[CG]] | | | | | | |
| | | | | | | | | | | | | | GCTGC | | | | | | |
| | | | | | | | | | | | | | [[CG]] | | | | | | |
| | | | | | | | | | | | | | GAGTT | | | | | | |
| | | | | | | | | | | | | | [[CG]] | | | | | | |
| SERTM1_ii | SERTM1 | 0.82 | 0.65 | 0.75 | 0.55 | chr13: | nega- | 13 | 372482 | 372483 | chr13: | 84 | [[CG]] | 372482 | 372483 | 372482 | 372482 | 372482 | 372482 |
| | | | | | | 37247945- | tive | | 38 | 21 | 37248238- | | GG | 97 | 21 | 38 | 56 | 70 | 95 |
| | | | | | | 37248544 | | | | | 37248321 | | [[CG]] | | | | | | |
| | | | | | | | | | | | | | [[CG]] | | | | | | |
| | | | | | | | | | | | | | TAT | | | | | | |
| | | | | | | | | | | | | | [[CG]] | | | | | | |
| | | | | | | | | | | | | | TCCAGA | | | | | | |
| | | | | | | | | | | | | | C[CG]G | | | | | | |
| | | | | | | | | | | | | | AGCAC | | | | | | |
| | | | | | | | | | | | | | [CG]CC | | | | | | |
| | | | | | | | | | | | | | CCAC | | | | | | |
| | | | | | | | | | | | | | [[CG]] | | | | | | |
| | | | | | | | | | | | | | CTAG | | | | | | |
| | | | | | | | | | | | | | [[CG]] | | | | | | |
| | | | | | | | | | | | | | CAGGAG | | | | | | |
| | | | | | | | | | | | | | ACCTGC | | | | | | |
| | | | | | | | | | | | | | [[CG]] | | | | | | |
| | | | | | | | | | | | | | GGGAAG | | | | | | |
| | | | | | | | | | | | | | T | | | | | | |
| | | | | | | | | | | | | | [[CG]] | | | | | | |
| | | | | | | | | | | | | | [[CG]] | | | | | | |
| | | | | | | | | | | | | | TGTCCT | | | | | | |
| | | | | | | | | | | | | | GACCTG | | | | | | |
| | | | | | | | | | | | | | CAG | | | | | | |
| LINC00403_ | SOX1-OT | 0.9 | 0.11 | 0.75 | 0.86 | chr13: | posi- | 13 | 1127119 | 1127120 | chr13: | 86 | TGAGC | 1127119 | 1127119 | 1127120 | 1127120 | 1127119 | 1127120 |
| i | | | | | | 112711710- | tive | | 67 | 52 | 112711967- | | [[CG]] | 67 | 92 | 27 | 52 | 94 | 25 |
| | | | | | | 112712309 | | | | | 112712052 | | CAAGCC | | | | | | |
| | | | | | | | | | | | | | TTGGAC | | | | | | |
| | | | | | | | | | | | | | T | | | | | | |
| | | | | | | | | | | | | | [[CG]] | | | | | | |
| | | | | | | | | | | | | | CAGAGC | | | | | | |
| | | | | | | | | | | | | | TGC | | | | | | |
| | | | | | | | | | | | | | [[CG]] | | | | | | |
| | | | | | | | | | | | | | GTGCC | | | | | | |
| | | | | | | | | | | | | | [[CG]] | | | | | | |
| | | | | | | | | | | | | | TC | | | | | | |
| | | | | | | | | | | | | | [[CG]] | | | | | | |
| | | | | | | | | | | | | | AGAGCC | | | | | | |
| | | | | | | | | | | | | | CCACCA | | | | | | |
| | | | | | | | | | | | | | G | | | | | | |
| | | | | | | | | | | | | | [[CG]] | | | | | | |
| | | | | | | | | | | | | | [[CG]] | | | | | | |
| | | | | | | | | | | | | | GCTCA | | | | | | |
| | | | | | | | | | | | | | [[CG]] | | | | | | |
| | | | | | | | | | | | | | CCTCAG | | | | | | |
| | | | | | | | | | | | | | TCT | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | CCCC | | | | | | |
| PDEIC_ii | PDE1C | 0.92 | 3.26 | 0.75 | 0.9 | chr7: | nega- | 7 | 324676 | 324677 | chr7: | 81 | [[CG]] | 324676 | 324677 | 324676 | 324676 | 324676 | 324676 |
| | | | | | | 32467358- | tive | | 24 | 04 | 32467624- | | [[CG]] | 82 | 04 | 24 | 44 | 48 | 80 |
| | | | | | | 32467957 | | | | | 32467704 | | AATGGG | | | | | | |
| | | | | | | | | | | | | | TCTC | | | | | | |
| | | | | | | | | | | | | | [[CG]] | | | | | | |
| | | | | | | | | | | | | | ACAGCA | | | | | | |
| | | | | | | | | | | | | | A[CG]G | | | | | | |
| | | | | | | | | | | | | | GAGCAG | | | | | | |
| | | | | | | | | | | | | | C | | | | | | |
| | | | | | | | | | | | | | [[CG]] | | | | | | |
| | | | | | | | | | | | | | GTGG | | | | | | |
| | | | | | | | | | | | | | [[CG]] | | | | | | |
| | | | | | | | | | | | | | CCTCAG | | | | | | |
| | | | | | | | | | | | | | GCTG | | | | | | |
| | | | | | | | | | | | | | [[CG]] | | | | | | |
| | | | | | | | | | | | | | GTGGCA | | | | | | |
| | | | | | | | | | | | | | A | | | | | | |
| | | | | | | | | | | | | | [[CG]] | | | | | | |
| | | | | | | | | | | | | | AGCC | | | | | | |
| | | | | | | | | | | | | | [[CG]] | | | | | | |
| | | | | | | | | | | | | | ACTGCA | | | | | | |
| | | | | | | | | | | | | | CTA | | | | | | |
| | | | | | | | | | | | | | [[CG]] | | | | | | |
| | | | | | | | | | | | | | GC | | | | | | |
| REG39_i | N/A | 0.86 | 5.32 | 0.75 | 0.8 | chr19: | posi- | 19 | 224445 | 224146 | chr19: | 71 | [[CG]] | 224445 | 224446 | 224446 | 224446 | 224446 | 224446 |
| | | | | | | 22444294- | tive | | 93 | 63 | 22444593- | | GACT | 93 | 12 | 40 | 63 | 14 | 38 |
| | | | | | | 22444893 | | | | | 22444663 | | [[CG]] | | | | | | |
| | | | | | | | | | | | | | GAGC | | | | | | |
| | | | | | | | | | | | | | [[CG]] | | | | | | |
| | | | | | | | | | | | | | TCCCTG | | | | | | |
| | | | | | | | | | | | | | CCAGTG | | | | | | |
| | | | | | | | | | | | | | GCTTT | | | | | | |
| | | | | | | | | | | | | | [[CG]] | | | | | | |
| | | | | | | | | | | | | | TGGG | | | | | | |
| | | | | | | | | | | | | | [[CG]] | | | | | | |
| | | | | | | | | | | | | | GCCC | | | | | | |
| | | | | | | | | | | | | | [[CG]] | | | | | | |
| | | | | | | | | | | | | | CCTTGG | | | | | | |
| | | | | | | | | | | | | | AGCCCA | | | | | | |
| | | | | | | | | | | | | | GGCCAA | | | | | | |
| | | | | | | | | | | | | | [[CG]] | | | | | | |
| | | | | | | | | | | | | | AGC | | | | | | |
| | | | | | | | | | | | | | [[CG]] | | | | | | |
| | | | | | | | | | | | | | C | | | | | | |
| RBFOX_i | RBFOX1 | 0.9 | 6.94 | 0.75 | 0.85 | chr16: | posi- | 16 | 60693 | 60694 | chr16: | 76 | [[CG]] | 60693 | 60693 | 60693 | 60694 | 60693 | 60693 |
| | | | | | | 6069102- | tive | | 28 | 03 | 6069328- | | CTGC | 28 | 53 | 81 | 03 | 55 | 79 |
| | | | | | | 6069701 | | | | | 6069403 | | [[CG]] | | | | | | |
| | | | | | | | | | | | | | C | | | | | | |
| | | | | | | | | | | | | | [[CG]] | | | | | | |
| | | | | | | | | | | | | | CCTCCT | | | | | | |
| | | | | | | | | | | | | | CCAGCC | | | | | | |
| | | | | | | | | | | | | | AGAGT | | | | | | |
| | | | | | | | | | | | | | [[CG]] | | | | | | |
| | | | | | | | | | | | | | GTGGGA | | | | | | |
| | | | | | | | | | | | | | CTGGCT | | | | | | |
| | | | | | | | | | | | | | G | | | | | | |
| | | | | | | | | | | | | | [[CG]] | | | | | | |
| | | | | | | | | | | | | | CTGCCC | | | | | | |
| | | | | | | | | | | | | | TGAAGT | | | | | | |
| | | | | | | | | | | | | | GGTTCT | | | | | | |
| | | | | | | | | | | | | | CCAAGC | | | | | | |
| | | | | | | | | | | | | | AG | | | | | | |
| | | | | | | | | | | | | | [[CG]] | | | | | | |
| | | | | | | | | | | | | | [[CG]] | | | | | | |
| | | | | | | | | | | | | | G | | | | | | |
| GALR1_i | GALR1 | 0.9 | 1.95 | 0.75 | 0.8 | chr18: | posi- | 18 | 749625 | 749626 | chr18: | 90 | GTGGAG | 749625 | 749626 | 749626 | 749626 | 749626 | 749626 |
| | | | | | | 74962373- | tive | | 95 | 84 | 74962595- | | AACTT | 95 | 17 | 66 | 84 | 20 | 44 |
| | | | | | | 74962972 | | | | | 74962684 | | [[CG]] | | | | | | |
| | | | | | | | | | | | | | TCA | | | | | | |
| | | | | | | | | | | | | | [[CG]] | | | | | | |
| | | | | | | | | | | | | | CTGGTG | | | | | | |
| | | | | | | | | | | | | | GTGTT | | | | | | |
| | | | | | | | | | | | | | [[CG]] | | | | | | |
| | | | | | | | | | | | | | GCCTGA | | | | | | |
| | | | | | | | | | | | | | TCTT | | | | | | |
| | | | | | | | | | | | | | [[CG]] | | | | | | |
| | | | | | | | | | | | | | [[CG]] | | | | | | |
| | | | | | | | | | | | | | CTGGGT | | | | | | |
| | | | | | | | | | | | | | GTGCTG | | | | | | |
| | | | | | | | | | | | | | GGCAAC | | | | | | |
| | | | | | | | | | | | | | AGCCTA | | | | | | |
| | | | | | | | | | | | | | GTGATC | | | | | | |
| | | | | | | | | | | | | | AC | | | | | | |
| | | | | | | | | | | | | | [[CG]] | | | | | | |
| | | | | | | | | | | | | | TGCTGG | | | | | | |
| | | | | | | | | | | | | | [[CG]] | | | | | | |
| | | | | | | | | | | | | | [[CG]] | | | | | | |
| | | | | | | | | | | | | | C | | | | | | |
| MDGA_I | MDGA2 | 0.92 | 4.73 | 0.75 | 0.85 | chr14: | posi- | 14 | 481440 | 481441 | chr14: | 115 | GGGTGC | 481440 | 481441 | 481441 | 481441 | 481441 | 481441 |
| | | | | | | 48143898- | tive | | 84 | 98 | 48144084- | | CTGGGA | 84 | 05 | 79 | 98 | 47 | 73 |
| | | | | | | 48144497 | | | | | 48144198 | | AAAT | | | | | | |
| | | | | | | | | | | | | | [[CG]] | | | | | | |
| | | | | | | | | | | | | | CAGA | | | | | | |
| | | | | | | | | | | | | | [CG]C | | | | | | |
| | | | | | | | | | | | | | [CG]GG | | | | | | |
| | | | | | | | | | | | | | GAGGAG | | | | | | |
| | | | | | | | | | | | | | CAGGGG | | | | | | |
| | | | | | | | | | | | | | G[CG]G | | | | | | |
| | | | | | | | | | | | | | TGATGG | | | | | | |
| | | | | | | | | | | | | | GAAGGG | | | | | | |
| | | | | | | | | | | | | | GAGCTG | | | | | | |
| | | | | | | | | | | | | | [[CG]] | | | | | | |
| | | | | | | | | | | | | | AGG | | | | | | |
| | | | | | | | | | | | | | [[CG]] | | | | | | |
| | | | | | | | | | | | | | AAGTGT | | | | | | |
| | | | | | | | | | | | | | TCTTCA | | | | | | |
| | | | | | | | | | | | | | GGGAAG | | | | | | |
| | | | | | | | | | | | | | [[CG]] | | | | | | |
| | | | | | | | | | | | | | GGCT | | | | | | |
| | | | | | | | | | | | | | [CG]AG | | | | | | |
| | | | | | | | | | | | | | TCTC | | | | | | |
| | | | | | | | | | | | | | [[CG]] | | | | | | |
| | | | | | | | | | | | | | CAGCTG | | | | | | |
| | | | | | | | | | | | | | [[CG]] | | | | | | |
| | | | | | | | | | | | | | G | | | | | | |
| | | | | | | | | | | | | | [[CG]] | | | | | | |
| FUT9_ii | FUT9 | 0.94 | 2.82 | 0.75 | 0.9 | chr6: | nega- | 6 | 964639 | 964640 | chr6: | 103 | [[CG]] | 964639 | 964640 | 964639 | 964639 | 964639 | 964639 |
| | | | | | | 96463603- | tive | | 02 | 04 | 96463902- | | CAGCAG | 84 | 04 | 02 | 25 | 48 | 70 |
| | | | | | | 96464202 | | | | | 96464004 | | CTCCAG | | | | | | |
| | | | | | | | | | | | | | ATTCAC | | | | | | |
| | | | | | | | | | | | | | TGCTCT | | | | | | |
| | | | | | | | | | | | | | CCCCTG | | | | | | |
| | | | | | | | | | | | | | CAGCTC | | | | | | |
| | | | | | | | | | | | | | CC[CG] | | | | | | |
| | | | | | | | | | | | | | [CG]CC | | | | | | |
| | | | | | | | | | | | | | CC | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | CTGT | | | | | | |
| | | | | | | | | | | | | | [[CG]] | | | | | | |
| | | | | | | | | | | | | | CTGCCT | | | | | | |
| | | | | | | | | | | | | | [[CG]] | | | | | | |
| | | | | | | | | | | | | | GTGTCC | | | | | | |
| | | | | | | | | | | | | | CCCAGC | | | | | | |
| | | | | | | | | | | | | | CCCAGT | | | | | | |
| | | | | | | | | | | | | | [[CG]] | | | | | | |
| | | | | | | | | | | | | | [[CG]] | | | | | | |
| | | | | | | | | | | | | | CTCTTA | | | | | | |
| | | | | | | | | | | | | | GGACAG | | | | | | |
| | | | | | | | | | | | | | [[CG]] | | | | | | |
| LHX8_i | LHX8 | 0.94 | 0 | 0.75 | 0.84 | chr1: | posi- | 1 | 756021 | 756021 | chr1: | 79 | GAAGGA | 756021 | 756021 | 756021 | 756021 | 756021 | 756021 |
| | | | | | | 75601868- | tive | | 07 | 85 | 75602107- | | GGCTG | 07 | 26 | 63 | 85 | 39 | 61 |
| | | | | | | 75602467 | | | | | 75602185 | | [[CG]] | | | | | | |
| | | | | | | | | | | | | | [[CG]] | | | | | | |
| | | | | | | | | | | | | | CCAGCC | | | | | | |
| | | | | | | | | | | | | | [CG]CC | | | | | | |
| | | | | | | | | | | | | | [CG] | | | | | | |
| | | | | | | | | | | | | | [CG]G | | | | | | |
| | | | | | | | | | | | | | [CG]CC | | | | | | |
| | | | | | | | | | | | | | [[CG]] | | | | | | |
| | | | | | | | | | | | | | GGCTCA | | | | | | |
| | | | | | | | | | | | | | GG | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | TGA | | | | | | |
| | | | | | | | | | | | | | [[CG]] | | | | | | |
| | | | | | | | | | | | | | GCTGCA | | | | | | |
| | | | | | | | | | | | | | [[CG]] | | | | | | |
| | | | | | | | | | | | | | [[CG]] | | | | | | |
| | | | | | | | | | | | | | CTGCCC | | | | | | |
| | | | | | | | | | | | | | [[CG]] | | | | | | |
| | | | | | | | | | | | | | CACTCT | | | | | | |
| | | | | | | | | | | | | | G | | | | | | |
| ELAVL4_i | ELAVL4 | 0.84 | 3.67 | 0.75 | 0.85 | chr1: | posi- | 1 | 505137 | 505137 | chr1: | 71 | [[CG]] | 505137 | 505137 | 505137 | 505137 | 505137 | 505137 |
| | | | | | | 50513450- | tive | | 18 | 88 | 50513718- | | GAGC | 18 | 40 | 70 | 88 | 43 | 65 |
| | | | | | | 50514049 | | | | | 50513788 | | [[CG]] | | | | | | |
| | | | | | | | | | | | | | CAGAG | | | | | | |
| | | | | | | | | | | | | | [[CG]] | | | | | | |
| | | | | | | | | | | | | | AGCTAG | | | | | | |
| | | | | | | | | | | | | | AGAG | | | | | | |
| | | | | | | | | | | | | | [[CG]] | | | | | | |
| | | | | | | | | | | | | | AGAG | | | | | | |
| | | | | | | | | | | | | | [[CG]] | | | | | | |
| | | | | | | | | | | | | | GTGAGA | | | | | | |
| | | | | | | | | | | | | | CTCTG | | | | | | |
| | | | | | | | | | | | | | [[CG]] | | | | | | |
| | | | | | | | | | | | | | GA[CG] | | | | | | |
| | | | | | | | | | | | | | TCTTCC | | | | | | |
| | | | | | | | | | | | | | [[CG]] | | | | | | |
| | | | | | | | | | | | | | CC | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | CTCC | | | | | | |
| NET01_i | NETO1 | 0.93 | 3.48 | 0.75 | 0.85 | chr18: | posi- | 18 | 705342 | 705343 | chr18: | 75 | AC | 705342 | 705342 | 705343 | 705343 | 705342 | 705343 |
| | | | | | | 70533999- | tive | | 72 | 46 | 70534272- | | [[CG]] | 72 | 93 | 23 | 46 | 95 | 15 |
| | | | | | | 70534598 | | | | | 70534346 | | TTCTC | | | | | | |
| | | | | | | | | | | | | | [[CG]] | | | | | | |
| | | | | | | | | | | | | | GCAGGT | | | | | | |
| | | | | | | | | | | | | | TTTGGG | | | | | | |
| | | | | | | | | | | | | | ATC | | | | | | |
| | | | | | | | | | | | | | [[CG]] | | | | | | |
| | | | | | | | | | | | | | G | | | | | | |
| | | | | | | | | | | | | | [[CG]] | | | | | | |
| | | | | | | | | | | | | | A | | | | | | |
| | | | | | | | | | | | | | [[CG]] | | | | | | |
| | | | | | | | | | | | | | GCTGAC | | | | | | |
| | | | | | | | | | | | | | [[CG]] | | | | | | |
| | | | | | | | | | | | | | [[CG]] | | | | | | |
| | | | | | | | | | | | | | [CG]C | | | | | | |
| | | | | | | | | | | | | | [CG]CC | | | | | | |
| | | | | | | | | | | | | | CCCA | | | | | | |
| | | | | | | | | | | | | | [[CG]] | | | | | | |
| | | | | | | | | | | | | | CC | | | | | | |
| | | | | | | | | | | | | | [[CG]] | | | | | | |
| | | | | | | | | | | | | | GTTCCA | | | | | | |
| | | | | | | | | | | | | | [[CG]] | | | | | | |
| | | | | | | | | | | | | | ATGCTG | | | | | | |
| RNF291_ii | RNF219- | 0.88 | 5.01 | 0.75 | 0.75 | chr13: | nega- | 13 | 791702 | 791703 | chr13: | 84 | GGCCTA | 791703 | 791703 | 791702 | 791703 | 791703 | 791703 |
| | AS1 | | | | | 79170004- | tive | | 84 | 67 | 79170284- | | AGGC | 41 | 67 | 84 | 08 | 14 | 38 |
| | | | | | | 79170603 | | | | | 79170367 | | [[CG]] | | | | | | |
| | | | | | | | | | | | | | TTGACC | | | | | | |
| | | | | | | | | | | | | | T | | | | | | |
| | | | | | | | | | | | | | [[CG]] | | | | | | |
| | | | | | | | | | | | | | GGTTCT | | | | | | |
| | | | | | | | | | | | | | CCC | | | | | | |
| | | | | | | | | | | | | | [[CG]] | | | | | | |
| | | | | | | | | | | | | | GCACAG | | | | | | |
| | | | | | | | | | | | | | T | | | | | | |
| | | | | | | | | | | | | | [[CG]] | | | | | | |
| | | | | | | | | | | | | | AATCCA | | | | | | |
| | | | | | | | | | | | | | [[CG]] | | | | | | |
| | | | | | | | | | | | | | CCAGGG | | | | | | |
| | | | | | | | | | | | | | CCCTCA | | | | | | |
| | | | | | | | | | | | | | GGC | | | | | | |
| | | | | | | | | | | | | | [[CG]] | | | | | | |
| | | | | | | | | | | | | | GTAGCT | | | | | | |
| | | | | | | | | | | | | | GTCCTG | | | | | | |
| | | | | | | | | | | | | | CAGTCC | | | | | | |
| LINC00403_ | SOX1- | 0.94 | 2.97 | 0.75 | 0.85 | chr13: | nega- | 13 | 1127172 | 1127173 | chr13: | 85 | CC | 1127173 | 1127173 | 1127172 | 1127172 | 1127172 | 1127173 |
| iv | OT | | | | | 112716945- | tive | | 33 | 17 | 112717233- | | [[CG]] | 03 | 17 | 33 | 51 | 83 | 01 |
| | | | | | | 112717544 | | | | | 112717317 | | GTGTCT | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | AGGG | | | | | | |
| | | | | | | | | | | | | | [CG]G | | | | | | |
| | | | | | | | | | | | | | [CG]G | | | | | | |
| | | | | | | | | | | | | | [CG]GC | | | | | | |
| | | | | | | | | | | | | | CAGCAG | | | | | | |
| | | | | | | | | | | | | | A[CG]G | | | | | | |
| | | | | | | | | | | | | | [CG]AT | | | | | | |
| | | | | | | | | | | | | | [CG]AG | | | | | | |
| | | | | | | | | | | | | | G[CG] | | | | | | |
| | | | | | | | | | | | | | [[CG]] | | | | | | |
| | | | | | | | | | | | | | [[CG]] | | | | | | |
| | | | | | | | | | | | | | CCA | | | | | | |
| | | | | | | | | | | | | | [[CG]] | | | | | | |
| | | | | | | | | | | | | | GCA | | | | | | |
| | | | | | | | | | | | | | [[CG]] | | | | | | |
| | | | | | | | | | | | | | GCCAG | | | | | | |
| | | | | | | | | | | | | | [CG]CA | | | | | | |
| | | | | | | | | | | | | | GACA | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | [[CG]] | | | | | | |
| | | | | | | | | | | | | | G | | | | | | |
| MHC_ii | N/A | 0.92 | 1.43 | 0.75 | 0.8 | chr6: | nega- | 6 | 295213 | 295214 | chr6: | 85 | GCC | 295214 | 295214 | 295213 | 295213 | 295214 | 295214 |
| | | | | | | 29521121- | tive | | 75 | 59 | 29521375- | | [[CG]] | 35 | 59 | 75 | 94 | 05 | 29 |
| | | | | | | 29521720 | | | | | 29521459 | | CTG | | | | | | |
| | | | | | | | | | | | | | [[CG]] | | | | | | |
| | | | | | | | | | | | | | CTATG | | | | | | |
| | | | | | | | | | | | | | [[CG]] | | | | | | |
| | | | | | | | | | | | | | GGGCT | | | | | | |
| | | | | | | | | | | | | | [CG]TC | | | | | | |
| | | | | | | | | | | | | | TCCC | | | | | | |
| | | | | | | | | | | | | | [[CG]] | | | | | | |
| | | | | | | | | | | | | | [[CG]] | | | | | | |
| | | | | | | | | | | | | | CCTATG | | | | | | |
| | | | | | | | | | | | | | T | | | | | | |
| | | | | | | | | | | | | | [[CG]] | | | | | | |
| | | | | | | | | | | | | | CA | | | | | | |
| | | | | | | | | | | | | | [[CG]] | | | | | | |
| | | | | | | | | | | | | | CTGGCC | | | | | | |
| | | | | | | | | | | | | | AG[CG] | | | | | | |
| | | | | | | | | | | | | | CCTCCT | | | | | | |
| | | | | | | | | | | | | | GGCTAA | | | | | | |
| | | | | | | | | | | | | | G | | | | | | |
| | | | | | | | | | | | | | [[CG]] | | | | | | |
| | | | | | | | | | | | | | GCCTCA | | | | | | |
| | | | | | | | | | | | | | CCAACT | | | | | | |
| | | | | | | | | | | | | | C | | | | | | |
| NID2_i | NID2 | 0.92 | 4.51 | 0.75 | 0.85 | chr14: | posi- | 14 | 525361 | 525361 | chr14: | 70 | TGCAGG | 525361 | 525361 | 525361 | 525361 | 525361 | 525361 |
| | | | | | | 52535876- | tive | | 29 | 98 | 52536129- | | AGATGA | 29 | 54 | 82 | 98 | 56 | 78 |
| | | | | | | 52536475 | | | | | 52536198 | | GCTCAG | | | | | | |
| | | | | | | | | | | | | | [[CG]] | | | | | | |
| | | | | | | | | | | | | | CAAAGG | | | | | | |
| | | | | | | | | | | | | | GAACCC | | | | | | |
| | | | | | | | | | | | | | [[CG]] | | | | | | |
| | | | | | | | | | | | | | CAG | | | | | | |
| | | | | | | | | | | | | | [[CG]] | | | | | | |
| | | | | | | | | | | | | | G | | | | | | |
| | | | | | | | | | | | | | [[CG]] | | | | | | |
| | | | | | | | | | | | | | AGTG | | | | | | |
| | | | | | | | | | | | | | [[CG]] | | | | | | |
| | | | | | | | | | | | | | GCTGCT | | | | | | |
| | | | | | | | | | | | | | GGCCTG | | | | | | |
| | | | | | | | | | | | | | [[CG]] | | | | | | |
| | | | | | | | | | | | | | [[CG]] | | | | | | |
| | | | | | | | | | | | | | CTGTGG | | | | | | |
| ZIC4 | ZIC4 | 0.92 | 6.89 | 0.75 | 0.9 | chr3: | nega- | 3 | 1471087 | 1471088 | chr3: | 89 | GG | 1471088 | 1471088 | 1471087 | 1471088 | 1471088 | 1471088 |
| | | | | | | 147108544- | tive | | 96 | 84 | 147108796- | | [[CG]] | 59 | 84 | 96 | 21 | 24 | 54 |
| | | | | | | 147109143 | | | | | 147108884 | | ACA | | | | | | |
| | | | | | | | | | | | | | [[CG]] | | | | | | |
| | | | | | | | | | | | | | AGGGCA | | | | | | |
| | | | | | | | | | | | | | GA | | | | | | |
| | | | | | | | | | | | | | [[CG]] | | | | | | |
| | | | | | | | | | | | | | GTGTAG | | | | | | |
| | | | | | | | | | | | | | C[CG]A | | | | | | |
| | | | | | | | | | | | | | AT | | | | | | |
| | | | | | | | | | | | | | [[CG]] | | | | | | |
| | | | | | | | | | | | | | TAGCCA | | | | | | |
| | | | | | | | | | | | | | GAGCTG | | | | | | |
| | | | | | | | | | | | | | GG | | | | | | |
| | | | | | | | | | | | | | [[CG]] | | | | | | |
| | | | | | | | | | | | | | G | | | | | | |
| | | | | | | | | | | | | | [[CG]] | | | | | | |
| | | | | | | | | | | | | | G | | | | | | |
| | | | | | | | | | | | | | [[CG]] | | | | | | |
| | | | | | | | | | | | | | AG | | | | | | |
| | | | | | | | | | | | | | [[CG]] | | | | | | |
| | | | | | | | | | | | | | CC[CG] | | | | | | |
| | | | | | | | | | | | | | TGCACC | | | | | | |
| | | | | | | | | | | | | | TTCATG | | | | | | |
| | | | | | | | | | | | | | TGCTTA | | | | | | |
| | | | | | | | | | | | | | [[CG]] | | | | | | |
| | | | | | | | | | | | | | CAG | | | | | | |
| | | | | | | | | | | | | | [[CG]] | | | | | | |
| | | | | | | | | | | | | | A | | | | | | |
| RCN1_i | RCN1 | 0.92 | 3.93 | 0.75 | 0.85 | chr11: | posi- | 11 | 320089 | 320090 | chr11: | 84 | [[CG]] | 320089 | 320089 | 320090 | 320090 | 320089 | 320090 |
| | | | | | | 32008662- | tive | | 59 | 42 | 32008959- | | [[CG]] | 59 | 79 | 23 | 42 | 91 | 19 |
| | | | | | | 32009261 | | | | | 32009042 | | AAGGAA | | | | | | |
| | | | | | | | | | | | | | AGTGCT | | | | | | |
| | | | | | | | | | | | | | A | | | | | | |
| | | | | | | | | | | | | | [[CG]] | | | | | | |
| | | | | | | | | | | | | | AA[CG] | | | | | | |
| | | | | | | | | | | | | | TCAAAT | | | | | | |
| | | | | | | | | | | | | | GGC | | | | | | |
| | | | | | | | | | | | | | [[CG]] | | | | | | |
| | | | | | | | | | | | | | CCCCC | | | | | | |
| | | | | | | | | | | | | | [[CG]] | | | | | | |
| | | | | | | | | | | | | | C | | | | | | |
| | | | | | | | | | | | | | [[CG]] | | | | | | |
| | | | | | | | | | | | | | A | | | | | | |
| | | | | | | | | | | | | | [[CG]] | | | | | | |
| | | | | | | | | | | | | | CCATCT | | | | | | |
| | | | | | | | | | | | | | GCTCTG | | | | | | |
| | | | | | | | | | | | | | [[CG]] | | | | | | |
| | | | | | | | | | | | | | AAGCAG | | | | | | |
| | | | | | | | | | | | | | AAA | | | | | | |
| | | | | | | | | | | | | | [[CG]] | | | | | | |
| | | | | | | | | | | | | | G | | | | | | |
| | | | | | | | | | | | | | [[CG]] | | | | | | |
| | | | | | | | | | | | | | GCAGCT | | | | | | |
| | | | | | | | | | | | | | G | | | | | | |
| | | | | | | | | | | | | | [[CG]] | | | | | | |
| RP4_ii | N/A | 0.85 | 5.38 | 0.75 | 0.7 | chr12: | nega- | 12 | 1266760 | 1266761 | chr12: | 102 | GGGCAA | 1266761 | 1266761 | 1266760 | 1266760 | 1266760 | 1266760 |
| | | | | | | 126675749- | tive | | 38 | 39 | 126676038- | | GGGC | 11 | 39 | 38 | 59 | 63 | 94 |
| | | | | | | 126676348 | | | | | 126676139 | | [[CG]] | | | | | | |
| | | | | | | | | | | | | | GGGC | | | | | | |
| | | | | | | | | | | | | | [[CG]] | | | | | | |
| | | | | | | | | | | | | | GACACA | | | | | | |
| | | | | | | | | | | | | | GGAGCA | | | | | | |
| | | | | | | | | | | | | | G | | | | | | |
| | | | | | | | | | | | | | [[CG]] | | | | | | |
| | | | | | | | | | | | | | GGGATG | | | | | | |
| | | | | | | | | | | | | | C | | | | | | |
| | | | | | | | | | | | | | [[CG]] | | | | | | |
| | | | | | | | | | | | | | GGTG | | | | | | |
| | | | | | | | | | | | | | [[CG]] | | | | | | |
| | | | | | | | | | | | | | CTC | | | | | | |
| | | | | | | | | | | | | | [[CG]] | | | | | | |
| | | | | | | | | | | | | | AGGGTG | | | | | | |
| | | | | | | | | | | | | | TGGCCC | | | | | | |
| | | | | | | | | | | | | | C[CG]G | | | | | | |
| | | | | | | | | | | | | | GCTGTG | | | | | | |
| | | | | | | | | | | | | | [[CG]] | | | | | | |
| | | | | | | | | | | | | | GGGCTC | | | | | | |
| | | | | | | | | | | | | | AGAGCT | | | | | | |
| | | | | | | | | | | | | | GCTTGC | | | | | | |
| | | | | | | | | | | | | | TGGG | | | | | | |
| | | | | | | | | | | | | | [[CG]] | | | | | | |
| | | | | | | | | | | | | | C | | | | | | |
| RYR2_i | RYR2 | 0.92 | 4.15 | 0.75 | 0.85 | chr1: | posi- | 1 | 2372053 | 2372054 | chr1: | 67 | G | 2372053 | 2372053 | 2372053 | 2372054 | 2372053 | 2372053 |
| | | | | | | 237205085- | tive | | 44 | 10 | 237205344- | | [[CG]] | 44 | 61 | 88 | 10 | 68 | 86 |
| | | | | | | 237205684 | | | | | 237205410 | | CTGGGC | | | | | | |
| | | | | | | | | | | | | | CT | | | | | | |
| | | | | | | | | | | | | | [[CG]] | | | | | | |
| | | | | | | | | | | | | | GGTTTG | | | | | | |
| | | | | | | | | | | | | | G[CG]G | | | | | | |
| | | | | | | | | | | | | | CC | | | | | | |
| | | | | | | | | | | | | | [[CG]] | | | | | | |
| | | | | | | | | | | | | | GAGGAG | | | | | | |
| | | | | | | | | | | | | | [[CG]] | | | | | | |
| | | | | | | | | | | | | | GGCTG | | | | | | |
| | | | | | | | | | | | | | [[CG]] | | | | | | |
| | | | | | | | | | | | | | GATTAC | | | | | | |
| | | | | | | | | | | | | | CTGCAG | | | | | | |
| | | | | | | | | | | | | | CAG | | | | | | |
| | | | | | | | | | | | | | [[CG]] | | | | | | |
| | | | | | | | | | | | | | GGGAGC | | | | | | |
| | | | | | | | | | | | | | [[CG]] | | | | | | |

1. A method comprising assaying from a sample from an individual
the methylation status of a panel of:
i. one or more CpGs selected from a panel of CpGs identified in SEQ ID NOs 1 to 5000 wherein the CpGs are identified at nucleotide positions 61 to 62; and/or
ii. one or more CpGs selected from within a panel of one or more Differentially Methylated Regions (DMRs) defined by SEQ ID NOs 5001 to 5418, wherein the CpGs are denoted by CG.

2. A method according to claim 1, wherein the panel of one or more CpGs comprises:
i) at least 500 CpGs selected from the CpGs identified at nucleotide positions 61 to 62 in SEQ ID NOs 1 to 5000, and optionally wherein the panel of one or more CpGs comprises at least the CpGs identified in SEQ ID NOs 1 to 500 and identified at nucleotide positions 61 to 62; or
ii) at least 1000 CpGs selected from the CpGs identified at nucleotide positions 61 to 62 in SEQ ID NOs 1 to 5000, and optionally wherein the panel of one or more CpGs comprises at least the CpGs identified in SEQ ID NOs 1 to 1000 and identified at nucleotide positions 61 to 62; or
iii) at least 1500 CpGs selected from the CpGs identified at nucleotide positions 61 to 62 in SEQ ID NOs 1 to 5000, and optionally wherein the panel of one or more CpGs comprises at least the CpGs identified in SEQ ID NOs 1 to 1500 and identified at nucleotide positions 61 to 62; or
iv) at least 2000 CpGs selected from the CpGs identified at nucleotide positions 61 to 62 in SEQ ID NOs 1 to 5000, and optionally wherein the panel of one or more CpGs comprises at least the CpGs identified in SEQ ID NOs 1 to 2000 and identified at nucleotide positions 61 to 62; or
v) at least the 5000 CpGs identified at nucleotide positions 61 to 62 in SEQ ID NOs 1 to 5000.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A method according to claim 1, wherein the step of assaying the methylation status of a panel of one or more CpGs comprises assaying the methylation status of one or more CpGs denoted by CG identified in a panel of one or more DMRs defined by SEQ ID NOs 5001 to 5418, optionally wherein the panel of one or more CpGs comprises two or more CpGs denoted by CG identified in the panel of DMR(s), three or more CpGs denoted by CG identified in the panel of DMR(s), four or more CpGs denoted by CG identified in the panel of DMR(s), or all CpGs denoted by CG identified in the DMR(s) defined by SEQ ID NOs 5001 to 5418.

17. A method according to claim 1, wherein the step of assaying the methylation status of a panel of the one or more CpGs comprises assaying the methylation status of five or more, six or more, seven or more, eight or more, or nine or more, or all of the CpGs denoted by CG within any one or more of the DMRs defined by SEQ ID NOs 5001 to 5418.

18. A method according to claim 1, wherein the step of assaying the methylation status of a panel of one or more CpGs comprises assaying the methylation status of two or more, three or more, four or more, five or more, six or more, seven or more, eight or more, or nine or more, or all of the CpGs denoted by CG within:
a. any combination of two, three, four, five, six, seven, eight, or nine or more of DMRs 1 to 418;
b. any combination of ten, twenty, thirty, forty, fifty, sixty, seventy, eighty, or ninety or more of DMRs 1 to 418;
c. all 418 of DMRs 1 to 418;
d. one DMR defined by SEQ ID NO: 5001, two DMRs defined by SEQ ID NOs: 5001 to 5002, three DMRs defined by SEQ ID NOs: 5001 to 5003, four DMRs defined by SEQ ID NOs: 5001 to 5004, five DMRs defined by SEQ ID NOs: 5001 to 5005, six DMRs defined by SEQ ID NOs: 5001 to 5006, seven DMRs defined by SEQ ID NOs: 5001 to 5007, eight DMRs defined by SEQ ID NOs: 5001 to 5008, or nine DMRs defined by SEQ ID NOs: 5001 to 5009;
e. any combination of one or more DMRs defined by SEQ ID NO: 5391, SEQ ID NO: 5392, SEQ ID NO: 5393, SEQ ID NO: 5407 and SEQ ID NO: 5414, preferably within all of SEQ ID NO: 5391, SEQ ID NO: 5392, SEQ ID NO: 5393, SEQ ID NO: 5407 and SEQ ID NO: 5414; f. any combination of one or more DMRs defined by SEQ ID NO: 5392, SEQ ID NO: 5393 and SEQ ID NO: 5407, preferably within all of SEQ ID NO: 5392, SEQ ID NO: 5393 and SEQ ID NO: 5407; or g. ten DMRs defined by SEQ ID NOs: 5001 to 5010, twenty DMRs defined by SEQ ID NOs: 5001 to 5020, thirty DMRs defined by SEQ ID NOs: 5001 to 5030, forty DMRs defined by SEQ ID NOs: 5001 to 5040, fifty DMRs defined by SEQ ID NOs: 5001 to 5050, sixty DMRs defined by
SEQ ID NOs: 5001 to 5060, seventy DMRs defined by SEQ ID NOs: 5001 to 5070, eighty DMRs defined by SEQ ID NOs: 5001 to 5080, or ninety DMRs defined by SEQ ID NOs: 5001 to 5090;
h. fifty DMRs defined by SEQ ID NOs: 5001 to 5050, SEQ ID NOs: 5051 to 5100, SEQ ID NOs: 5101 to 5150, SEQ ID NOs: 5151 to 5200, SEQ ID NOs: 5201 to 5250, SEQ ID NOs: 5301 to 5350, or SEQ ID NOs: 5341 to 5418; or
i. eighty one DMRs defined by SEQ ID NOs: 5015, 5016, 5017, 5025, 5026, 5027, 5028, 5029, 5032, 5033, 5048, 5049, 5050, 5053, 5054, 5057, 5068, 5069, 5071, 5072, 5073, 5074, 5075, 5076, 5077, 5083, 5090, 5091, 5092, 5093, 5094, 5095, 5096, 5099, 5102, 5137, 5138, 5140, 5143, 5146, 5147, 5148, 5149, 5150, 5151, 5164, 5165, 5167, 5175, 5176, 5177, 5179, 5180, 5185, 5204, 5224, 5226, 5228, 5232, 5246, 5248, 5285, 5287, 5293, 5307, 5309, 5315, 5317, 5324, 5328, 5337, 5339, 5340, 5348, 5349, 5354, 5361, 5362, 5366, 5368 and 5377.

19. A method according to claim 1, wherein the step of assaying the methylation status of a panel of one or more CpGs comprises assaying the methylation status of one or more CpGs within any one or more DMRs selected from the group of DMRs consisting of DMRs 1 to 418 as defined by SEQ ID NOs 5001 to 5418, including:
1. one or more CpGs within DMR 1 as defined by SEQ ID NO: 5001 and denoted by CG, preferably wherein the panel of one or more CpGs comprises at least the CpGs denoted by [[CG]];
2. one or more CpGs within DMR 2 as defined by SEQ ID NO: 5002 and denoted by CG, preferably wherein the panel of one or more CpGs comprises at least the CpGs denoted by [[CG]];
3. one or more CpGs within DMR 3 as defined by SEQ ID NO: 5003 and denoted by CG, preferably wherein the panel of one or more CpGs comprises at least the CpGs denoted by [[CG]];
4. one or more CpGs within DMR 4 as defined by SEQ ID NO: 5004 and denoted by CG, preferably wherein the panel of one or more CpGs comprises at least the CpGs denoted by [[CG]];
5. one or more CpGs within DMR 5 as defined by SEQ ID NO: 5005 and denoted by CG, preferably wherein the panel of one or more CpGs comprises at least the CpGs denoted by [[CG]];
6. one or more CpGs within DMR 6 as defined by SEQ ID NO: 5006 and denoted by CG, preferably wherein the panel of one or more CpGs comprises at least the CpGs denoted by [[CG]];
7. one or more CpGs within DMR 7 as defined by SEQ ID NO: 5007 and denoted by CG, preferably wherein the panel of one or more CpGs comprises at least the CpGs denoted by [[CG]];
8. one or more CpGs within DMR 8 as defined by SEQ ID NO: 5008 and denoted by CG, preferably wherein the panel of one or more CpGs comprises at least the CpGs denoted by [[CG]];
9. one or more CpGs within DMR 9 as defined by SEQ ID NO: 5009 and denoted by CG, preferably wherein the panel of one or more CpGs comprises at least the CpGs denoted by [[CG]]; and/or
10. one or more CpGs within DMR 10 as defined by SEQ ID NO: 5010 and denoted by CG, preferably wherein the panel of one or more CpGs comprises at least the CpGs denoted by [[CG]].

20. (canceled)

21. (canceled)

22. A method according to claim 1, wherein the step of assaying the methylation status of the one or more CpGs in the panel further comprises or additionally comprises assaying the methylation status of each CpG within one or more of the sequences identified by SEQ ID NOs 5703 to 5786, and optionally wherein the step of assaying the methylation status of the one or more CpGs in the panel comprises assaying each CpG within:
a. SEQ ID NO 5759 and/or SEQ ID NO 5703 and/or SEQ ID NO 5731;
b. SEQ ID NO 5760 and/or SEQ ID NO 5704 and/or SEQ ID NO 5732;
c. SEQ ID NO 5761 and/or SEQ ID NO 5705 and/or SEQ ID NO 5733;
d. SEQ ID NO 5762 and/or SEQ ID NO 5706 and/or SEQ ID NO 5734; and/or
e. SEQ ID NO 5763 and/or SEQ ID NO 5707 and/or SEQ ID NO 5735.

23. (canceled)

24. A method according to claim 1, wherein:
i) the step of assaying the methylation status of each CpG in the panel of one or more CpGs comprises:
a. performing a sequencing step to determine the sequence of each CpG;
b. hybridising DNA to an array comprising probes capable of discriminating between methylated and non-methylated forms of the CpGs and applying a detection system to the array so as to determine the methylation status of each CpG; and/or
c. performing a PCR step using methylation-specific primers, wherein the methylation status of the CpG is determined by the presence or absence of a PCR product; and/or
ii) the step of assaying the methylation status of each CpG in the panel of one or more CpGs comprises:
a. bisulphite converting the DNA; or
b. performing the steps of oxidising 5-methylcytosine bases (5mC) to 5-carboxylcytosine bases (5caC), preferably by ten-eleven translocation (TET), and/or oxidising 5-hydroxymethylcytosine bases (5hmC) to 5-carboxylcytosine bases (5caC), preferably by ten-eleven translocation (TET); followed by reducing 5-carboxylcytosine bases (5caC) to dihydrouracil bases (DHU), optionally with pyridine borane.

25. A method according to claim 24, further defined as comprising a step of stratifying the individual according to their risk of having CIN3 and/or cervical or according to their risk of CIN3 and/or cervical cancer development.

26. A method of treating or preventing CIN3 and/or cervical cancer in an individual, the method comprising administering one or more treatments to the individual determined to have CIN3 and/or cervical cancer by the step of performing the method of claim 1 on a sample from the individual.

27.-47. (canceled)

48. A method according to claim 9, further defined as comprising a step of stratifying the individual according to their risk of having CIN3 and/or cervical or according to their risk of CIN3 and/or cervical cancer development.

49. A method according to claim 48, wherein the individual is stratified as not having CIN3 and/or cancer or as having a low risk of CIN3 and/or cancer development, and wherein the individual is subjected to one or more treatments according to their stratification, the one or more treatments comprising a repeat method according to claim 10, preferably wherein the repeat method is performed about one year after the previous assay.

50. A method according to claim 48, wherein the individual is stratified as having a moderate risk of having CIN3 and/or cancer or as having a moderate risk of CIN3 and/or cancer development, and wherein the individual is subjected to one or more treatments according to their stratification, the one or more treatments comprising a test for human papilloma virus (HPV) status and wherein:
a. when the individual is HPV positive, a colposcopy, and optionally a transvaginal ultrasound and/or an endometrial biopsy to assess endometrium; or
b. when the individual is HPV negative, a repeat assay according to claim 48, preferably wherein the repeat assay is performed about one year after the previous assay.

51. A method according to claim 48, wherein the individual is stratified as having CIN3 and/or cancer or as having a high risk of CIN3 and/or cancer development, and wherein the individual is subjected to one or more treatments according to their stratification, the one or more treatments comprising a colposcopy, and wherein the colposcopy is negative, an endometrial biopsy and hysteroscopy.

52. A method according to claim 26, wherein the one or more treatments that the individual is subjected to are repeated on a monthly, three monthly, six monthly, yearly or two yearly basis following an initial administration.

53. A method of assaying methylation in an individual at multiple time points, the method comprising: (a) performing the assay according to claim 1 at a first time point; (b) performing the assay according claim 1 at one or more further time points; and (c) detecting differential methylation status between (a) and (b).

54. A method according to claim 53, wherein the further time points are monthly, three monthly, six monthly, yearly or two yearly basis following an initial assessment; and/or wherein one or more treatments are administered to the individual according to claim 51.

55. A method according to claim 1, wherein the sample is obtained from a tissue comprising epithelial cells, preferably wherein the sample is not obtained from ovarian or endometrial tissue, optionally wherein the sample is obtained from:
a. cervical tissue;
b. vaginal tissue;
c. cervicovaginal tissue;
d. buccal tissue;
preferably wherein the sample is obtained from cervical tissue, most preferably wherein the sample is obtained from tissue from a cervical smear.

56. An array for discriminating between methylated and non-methylated forms of CpGs; the array comprising oligonucleotide probes specific for a methylated form of each CpG in a CpG panel and oligonucleotide probes specific for a non-methylated form of each CpG in the panel; wherein the panel comprises at least 500 CpGs selected from the CpGs identified in SEQ ID NOs 1 to 5000 and identified at nucleotide positions 61 to 62, and identified in SEQ ID NOs 5001 to 5418 and denoted by CG, optionally provided that the array is not an Infinium MethylationEPIC BeadChip array or an Infinium HumanMethylation450, and/or provided that the number of CpG-specific oligonucleotide probes of the array is 482,000 or less, 480,000 or less, 450,000 or less, 440,000 or less, 430,000 or less, 420,000 or less, 410,000 or less, or 400,000 or less, and further optionally
wherein the panel comprises any panel of CpGs defined in a method of assaying from a sample from an individual the methylation status of a panel of CpGs identified in SEQ ID NOs 1 to 5000 wherein the CpGs are identified at nucleotide positions 61 to 62; and/or one or more CpGs selected from within panel of one or more Differentially Methylated Regions (DMRs) defined by SEQ ID NOs 5001 to 5418, wherein the CpGs are denoted by CG.

57. A hybridized array, wherein the array is obtainable by hybridizing to an array according to claim 56 to a group of oligonucleotides comprising any panel of CpGs defined in a method of assaying from a sample from an individual the methylation status of a panel of CpGs identified in SEQ ID NOs 1 to 5000 wherein the CpGs are identified at nucleotide positions 61 to 62; and/or one or more CpGs selected from within panel of one or more Differentially Methylated Regions (DMRs) defined by SEQ ID NOs 5001 to 5418, wherein the CpGs are denoted by CG.

58. A process for making the hybridized array according to claim 57, comprising contacting an array comprising oligonucleotide probes specific for a methylated form of each CpG in a CpG panel and oligonucleotide probes specific for a non-methylated form of each CpG in the panel; wherein the panel consists of at least 500 CpGs selected from the CpGs identified in SEQ ID NOs 1 to 5000 and identified at nucleotide positions 61 to 62, and identified in SEQ ID NOs 5001 to 5418 and denoted by CG in SEQ ID NO with a group of oligonucleotides comprising any panel of CpGs defined in the method of assaying from a sample from an individual the methylation status of a panel of CpGs identified in SEQ ID NOs 1 to 5000 wherein the CpGs are identified at nucleotide positions 61 to 62; and/or one or more CpGs selected from within panel of one or more Differentially Methylated Regions (DMRs) defined by SEQ ID NOs 5001 to 5418, wherein the CpGs are denoted by CG.
